(19) United States
(12) Patent Application Publication
(10) Pub. No.: US 2026/0050213 A1
(43) Pub. Date: Feb. 19, 2026

(54) ONIUM SALT, CHEMICALLY AMPLIFIED RESIST COMPOSITION, AND PATTERNING PROCESS

(71) Applicant: SHIN-ETSU CHEMICAL CO., LTD., Tokyo (JP)

(72) Inventor: Masahiro FUKUSHIMA, Joetsu-shi (JP)

(73) Assignee: SHIN-ETSU CHEMICAL CO., LTD., Tokyo (JP)

(21) Appl. No.: 19/293,361

(22) Filed: Aug. 7, 2025

(30) Foreign Application Priority Data
Aug. 13, 2024 (JP) 2024-134885

(51) Int. Cl. G03F 7/004; C07C 381/12; C08F 20/10; G03F 7/00; G03F 7/039

(52) U.S. Cl. CPC G03F 7/0045; C07C 381/12; C08F 20/10; G03F 7/0046; G03F 7/0397; G03F 7/70033

(57) ABSTRACT

The present invention is an onium salt composed of: an anion represented by general formula (1A); and a cation represented by general formula (1B). This can provide an onium salt used as a photoacid generator contained in a chemically amplified resist composition with excellent solvent solubility, high sensitivity, high contrast, and excellent lithography performance in photolithography using high-energy radiation; a photoacid generator composed of the onium salt; a chemically amplified resist composition containing the photoacid generator; and a patterning process using the chemically amplified resist composition.

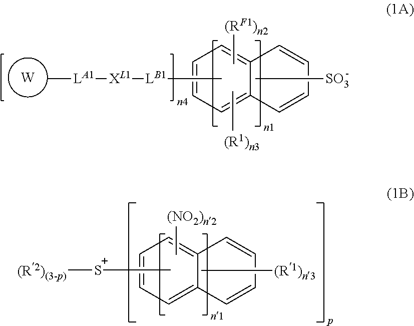

### TECHNICAL FIELD

The present invention relates to an onium salt, a chemically amplified resist composition, and a patterning process.

### BACKGROUND ART

To meet the recent demand for a finer pattern rule in the drive for high integration and high-speed processing in LSI, deep-ultraviolet lithography and extreme ultraviolet (EUV) lithography are considered promising as the next generation in microfabrication technology.

Photolithography using ArF excimer laser (ArF lithography) has started to be used partially from the fabrication of 130-nm node devices and become the main lithography since 90-nm node devices. Lithography using F₂ laser having a wavelength of 157 nm has initially been considered promising as the next lithography for 45-nm node devices. Due to development delays caused by various issues, however, ArF immersion lithography that introduces a liquid having a higher refractive index than air, such as water, ethylene glycol, and glycerol, between the projection lens and the wafer to allow the projection lens to be designed to a numerical aperture (NA) of 1.0 or higher, achieving a higher resolution, has been put into practical use. The immersion lithography requires a resist composition which is substantially insoluble in water.

In the ArF lithography, a highly sensitive resist composition capable of achieving sufficient resolution at a small dose of exposure is required to prevent the degradation of precise and expensive optical system materials. The most common method of providing the above is to select components highly transparent at a wavelength of 193 nm. For example, polyacrylic acid and a derivative thereof, a norbornene-maleic anhydride alternating copolymer, polynorbornene, a ring-opening metathesis polymer, and a hydrogenated ring-opening metathesis polymer have been proposed as the base polymer. This choice is effective to some extent in enhancing the transparency of a resin alone.

Recently a highlight has been put on the negative tone resist adapted for organic solvent development as well as the positive tone resist adapted for aqueous alkaline development. To resolve a very fine hole pattern, which is not achievable with the positive tone, through negative tone exposure, a highly resolvable positive resist composition is subjected to organic solvent development to form a negative pattern. Furthermore, an approach to doubling a resolution by combining two developments, aqueous alkaline development and organic solvent development, is currently under study. As an Arf resist composition for negative tone development with an organic solvent, a conventional positive Arf resist composition may be used, and patterning processes using the same are described in Patent Documents 1 to 3.

To accommodate the recent rapid progress of microfabrication technology, development efforts are put on not only the process but also the resist composition. Various studies have also been made on photoacid generators. Sulfonium salts of triphenylsulfonium cation with perfluoroalkanesulfonic acid anion are commonly used. These salts generate perfluoroalkanesulfonic acids, especially perfluorooctanesulfonic acid (PFOS), which are considered problematic with respect to their poor degradability, biological concentration, and toxicity, making their application in the resist composition rather restricted. Instead, photoacid generators that generate perfluorobutanesulfonic acids are currently used, but are awkward to achieve a high resolution because of substantial diffusion of the generated acids in the resist composition. To address the problem, various partially fluorinated alkane sulfonic acids and salts thereof have been developed. For example, Patent Document 1 describes as the prior art a photoacid generator that generates α,α-difluoroalkanesulfonic acid, specifically di(4-tert-butylphenyl)iodonium 1,1-difluoro-2-(1-naphthyl)ethanesulfonate, and a photoacid generator that generates α,α,β,β-tetrafluoroalkanesulfonic acid. Despite a reduced degree of fluorine substitution, however, these photoacid generators still have the following problems: since they do not have a decomposable substituent such as ester structure, the photoacid generators are unsatisfactory from the viewpoint of environmental safety due to ease of decomposition; the molecular design to change the size of alkanesulfonic acid is limited; and starting materials containing fluorine atoms are expensive.

As the circuit line width is reduced, the degradation of contrast by acid diffusion becomes more serious for the resist composition. The reason is that the pattern dimension is approaching the diffusion length of acid, leading to a lowering of mask fidelity and a degradation of pattern rectangularity because a dimensional shift on wafer (known as mask error factor (MEF)) relative to a dimensional shift on mask is exaggerated. Accordingly, to gain more benefits from shorter wavelength of light source and higher NA, the resist composition is required to increase a dissolution contrast or restrain acid diffusion, as compared with the prior art materials. One of the improvement measures is to lower the bake temperature for suppressing acid diffusion and hence, improving MEF. A low bake temperature, however, inevitably leads to a low sensitivity.

Incorporating a bulky substituent or polar group into a photoacid generator is effective for suppressing acid diffusion. Patent Document 4 describes a photoacid generator having 2-acyloxy-1,1,3,3,3-pentafluoropropane-1-sulfonic acid excellent in solubility and stability to solvent and capable of wide molecular design. In particular, a photoacid generator having 2-(1-adamantyloxy)-1,1,3,3,3-pentafluoropropane-1-sulfonic acid with a bulky substituent incorporated therein shows little acid diffusion. Patent Documents 5 to 7 describe photoacid generators having fused ring lactone, sultone or thiolactone incorporated as the polar group. Although some improvement in performance is observed due to the acid diffusion suppressing effect of the polar group incorporated, they are still insufficient in precise control of acid diffusion, failing to exert satisfactory lithography performance when evaluated totally in terms of MEF, pattern profile, and sensitivity.

Incorporating a polar group into an anion of a photoacid generator is effective for suppressing acid diffusion but disadvantageous from the viewpoint of solvent solubility. Attempting to improve solvent solubility, Patent Documents 8 and 9 propose to incorporate an alicyclic group into a cation moiety of a photoacid generator. Specifically, a cyclohexane ring or adamantane ring is incorporated. While incorporating such an alicyclic group achieves an improvement in solubility, a relatively large number of carbon atoms is necessary to ensure sufficient solubility. As a result, the molecular structure of the photoacid generator becomes bulky, causing a degradation of lithography performance such as line-width roughness (LWR) and critical dimension uniformity (CDU) during fine patterning.

Patent Document 10 describes a photoacid generator that generates fluoroalkanesulfonic acid with an aromatic fused ring derived from anthracene in an anion. Although improvement in lithography performance has been thus confirmed to some extent, the alkanesulfonic acid structure lacks rigidity. Recently, fluoroalkanesulfonic acid has been subject to regulations as a PFAS-related organic fluorine compound, raising concerns about its environmental and human health impact.

Iodine atoms are so absorptive to EUV having a wavelength of 13.5 nm that the iodine atoms have been observed to generate secondary electrons during expose, and this effect has gained attention in the EUV lithography. Patent Document 11 describes a photoacid generator having an iodine atom incorporated into an anion, and Patent Document 12 describes a polymerizable group-containing photoacid generator having an iodine atom incorporated into an anion. Patent Document 13 describes a photoacid generator having an iodine atom introduced into both a cation and an anion. Although improvement in lithography performance has been thus confirmed to some extent, the iodine atom is not highly soluble in an organic solvent, raising a concern about precipitation in a solvent.

Patent Document 14 describes a photoacid generator having a plurality of fluorine atoms incorporated into a cation. Although a plurality of fluorine atoms are incorporated to improve solvent solubility of the photoacid generator, there remains room for improvement since the solvent solubility is not sufficient from the viewpoint of EUV absorption.

Patent Documents 15 to 19 describe photoacid generators or quenchers (acid diffusion regulators) containing an iodine atom and a fluorine atom in a cation. Although these developments have confirmed improvements in performance as a resist material, the performance is still not satisfactory from the viewpoint of acid diffusion control, and further development of a resist material useful for fine patterning is required.

### CITATION LIST

### Patent Literature

Patent Document 1: JP 2008-281974 A
Patent Document 2: JP 2008-281975 A
Patent Document 3: JP 4554665 B
Patent Document 4: JP 2007-145797 A
Patent Document 5: JP 5061484 B
Patent Document 6: JP 2016-147879 A
Patent Document 7: JP 2015-63472 A
Patent Document 8: JP 5573098 B
Patent Document 9: JP 6461919 B
Patent Document 10: JP 7109178 B
Patent Document 11: JP 6720926 B
Patent Document 12: JP 6973274 B
Patent Document 13: JP 7041204 B
Patent Document 14: JP 7389562 B
Patent Document 15: JP 2021-123579 A
Patent Document 16: JP 2021-123580 A
Patent Document 17: JP 2022-123839 A
Patent Document 18: JP 2023-88869 A
Patent Document 19: JP 2023-88870 A

### Non Patent Literature

Non Patent Document 1: Journal of Photopolymer Science and Technology, Vol. 17, No. 4, p. 587-601 (2004)

### SUMMARY OF INVENTION

### Technical Problem

In response to the increasing demand for high-resolution resist patterns, a resist composition using a conventional onium salt-type photoacid generator fails to fully suppress acid diffusion. As a result, lithography performance, such as contrast, LWR, CDU, MEF, exposure latitude (EL), and depth of focus (DOF), may deteriorate.

In view of the aforementioned circumstances, the present invention aims to provide: an onium salt used as a photoacid generator contained in a chemically amplified resist composition with excellent solvent solubility, high sensitivity, high contrast, and excellent lithography performance in photolithography using high-energy radiation; a photoacid generator composed of the onium salt; a chemically amplified resist composition containing the photoacid generator; and a patterning process using the chemically amplified resist composition.

### Solution to Problem

To achieve the above object, the present invention provides an onium salt comprising: an anion represented by general formula (1A); and a cation represented by general formula (1B):

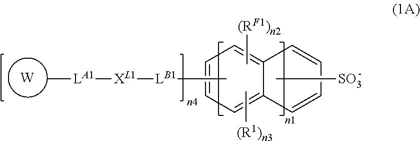

wherein “n1” is 0 or 1, “n2” is an integer of 0 to 4, “n3” is an integer of 0 to 4, provided that when “n1” is 0, 0≤n2+n3≤4, and when “n1” is 1, 0≤n2+n3≤6, and “n4” is 0 or 1;
W is a hydrocarbyl group having 6 to 60 carbon atoms and comprising at least one aromatic ring, wherein the hydrocarbyl group optionally comprises a heteroatom;
R^{F1} is a fluorine atom, a fluorinated saturated hydrocarbyl group having 1 to 6 carbon atoms, a fluorinated saturated hydrocarbyloxy group having 1 to 6 carbon atoms, or a fluorinated saturated hydrocarbylthio group having 1 to 6 carbon atoms;
R¹ is a halogen atom other than fluorine, a nitro group, a hydrocarbyl group having 1 to 20 carbon atoms and optionally comprising a heteroatom, a hydrocarbyloxy group having 1 to 20 carbon atoms and optionally comprising a heteroatom, or a hydrocarbylthio group having 1 to 20 carbon atoms and optionally comprising a heteroatom, wherein when “n3” is 2, 3, or 4, R¹ are the same as or different from each other, and a plurality of R¹ are optionally bonded to each other to form a ring together with carbon atoms to which the R¹ are bonded;
L^{A1} and L^{B1} are each independently a single bond, an ether bond, an ester bond, a sulfonate ester bond, an amide bond, a sulfonamide bond, a carbonate bond, or a carbamate bond;
X^{L1} is a single bond or a hydrocarbylene group having 1 to 40 carbon atoms and optionally comprising a heteroatom, and

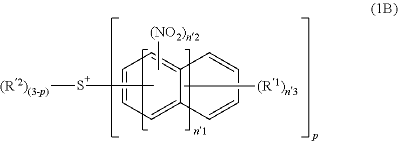

wherein “p” is 1, 2, or 3, “n′1” is 0 or 1, “n′2” is 1 or 2, and “n′3” is an integer of 0 to 6, provided that when “n′1” is 0, 1≤n′2+n′3≤5, and when “n′1” is 1, 1≤n′2+n′3≤7;
R′¹ is a halogen atom, a cyano group, a hydroxy group, a carboxy group, an alkoxycarbonyl group, a hydrocarbyl group having 1 to 20 carbon atoms and optionally comprising a heteroatom, a hydrocarbyloxy group having 1 to 20 carbon atoms and optionally comprising a heteroatom, or a hydrocarbylthio group having 1 to 20 carbon atoms and optionally comprising a heteroatom, wherein when “n′3” is 2 to 6, R′¹ are the same as or different from each other, and two R¹ are optionally bonded to each other to form a ring together with carbon atoms to which the R¹ are bonded; and
R′² is a halogen atom or a hydrocarbyl group having 1 to 30 carbon atoms and optionally comprising a heteroatom, wherein when “p” is 1, two R′² are the same as or different from each other, and two of three substituents bonded to S⁺ are optionally bonded to each other to form a ring together with a sulfur atom to which the substituents are bonded.

Such an onium salt has excellent solvent solubility, and a chemically amplified resist composition using the onium salt as a photoacid generator is extremely effective in suppressing acid diffusion with high sensitivity and high contrast, is excellent in lithography performance such as LWR, CDU, MEF, EL, DOF, and is extremely effective in fine patterning.

In this case, the W is preferably represented by general formula (W-1) or (W-2):

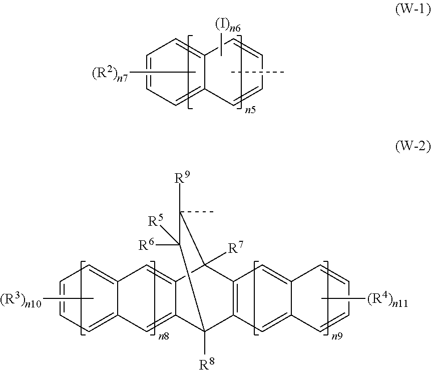

wherein “n5” is 0 or 1, “n6” is an integer of 0 to 4, “n7” is an integer of 1 to 4, provided that when “n5” is 0, 1≤n6+n7≤5, and when “n5” is 1, 1≤n6+n7≤7, “n8” is 0 or 1, “n9” is 0 or 1, “n10” is an integer of 0 to 4, and “n11” is an integer of 0 to 4;
each R² is independently a hydrogen atom, a halogen atom other than an iodine atom, or a hydrocarbyl group having 1 to 20 carbon atoms and optionally comprising a heteroatom;
R³ and R⁴ are each independently a hydrogen atom, a halogen atom, or a hydrocarbyl group having 1 to 20 carbon atoms and optionally comprising a heteroatom;
R⁵ to R⁹ are each independently a hydrogen atom, a halogen atom, or a hydrocarbyl group having 1 to 20 carbon atoms and optionally comprising a heteroatom; and the dashed line represents a bond to L^{A1}.

The anion contained in the onium salt of the present invention preferably has such a group.

In addition, the anion is preferably represented by general formula (1A-1):

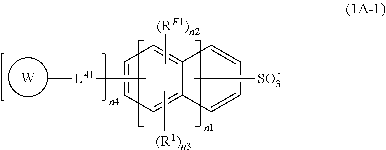

wherein “n1” to “n4”, W, R^{F1}, R¹, and L^{A1} are the same as above.

The onium salt of the present invention preferably has such an anion.

In addition, the cation is preferably represented by general formula (1B-1):

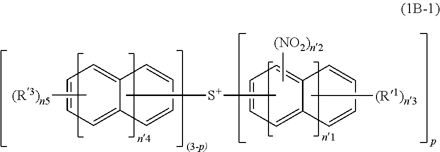

wherein “p”, “n′¹” to “n′³”, and R′¹ are the same as above;
“n′⁴” is 0 or 1, “n′⁵” is an integer of 0 to 5; and
R′³ is a halogen atom, a cyano group, a hydroxy group, a carboxy group, an alkoxycarbonyl group, a hydrocarbyl group having 1 to 20 carbon atoms and optionally comprising a heteroatom, a hydrocarbyloxy group having 1 to 20 carbon atoms and optionally comprising a heteroatom, or a hydrocarbylthio group having 1 to 20 carbon atoms and optionally comprising a heteroatom, wherein when “n′⁵” is 2 to 5, R′³ are the same as or different from each other, and two R′³ are optionally bonded to each other to form a ring together with carbon atoms to which the R′³ are bonded.

The onium salt of the present invention preferably has such a cation.

The present invention also provides a photoacid generator comprising the onium salt described above.

Such a photoacid generator is useful in the chemically amplified resist compositions of the present invention.

The present invention also provides a chemically amplified resist composition comprising the photoacid generator described above.

When patterning is performed using a chemically amplified resist composition containing such a photoacid generator, the chemically amplified resist composition exhibits high sensitivity, excellent acid diffusion suppression, and superior lithography performance, making it effective for fine patterning.

The chemically amplified resist composition preferably further contains a base polymer comprising a repeating unit represented by either or both of general formulae (a1) and (a2):

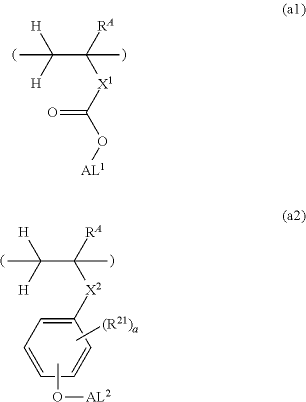

wherein each R^{A} is independently a hydrogen atom, a fluorine atom, a methyl group, or a trifluoromethyl group;
X¹ is a single bond, a phenylene group, a naphthylene group, or *—C(═O)—O—X¹¹—, wherein the phenylene group or naphthylene group is optionally substituted with a hydroxy group, a nitro group, a cyano group, a saturated hydrocarbyl group having 1 to 10 carbon atoms and optionally comprising a fluorine atom, a saturated hydrocarbyloxy group having 1 to 10 carbon atoms and optionally comprising a fluorine atom, or a halogen atom; X¹¹ is a saturated hydrocarbylene group having 1 to 10 carbon atoms, a phenylene group, or a naphthylene group, wherein the saturated hydrocarbylene group optionally comprises a hydroxy group, an ether bond, an ester bond, or a lactone ring;
X² is a single bond or *—C(═O)—O—;
“*” represents a bond to a carbon atom of a main chain;
R²¹ is a halogen atom, a cyano group, a hydroxy group, a nitro group, a hydrocarbyl group having 1 to 20 carbon atoms and optionally comprising a heteroatom, a hydrocarbyloxy group having 1 to 20 carbon atoms and optionally comprising a heteroatom, a hydrocarbylcarbonyl group having 2 to 20 carbon atoms and optionally comprising a heteroatom, a hydrocarbylcarbonyloxy group having 2 to 20 carbon atoms and optionally comprising a heteroatom, or a hydrocarbyloxycarbonyl group having 2 to 20 carbon atoms and optionally comprising a heteroatom;
A^{L1} and A^{L2} are each independently an acid labile group; and
“a” is an integer of 0 to 4.

The chemically amplified resist composition of the present invention may contain such a base polymer.

The chemically amplified resist composition preferably contains the base polymer further comprising a repeating unit represented by general formula (a3):

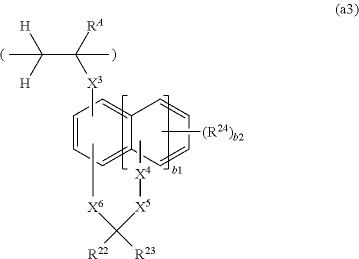

wherein “b1” is 0 or 1, “b2” is an integer of 0 to 3 when the “b1” is 0, and an integer of 0 to 5 when the “b1” is 1;
R^{A} is a hydrogen atom, a fluorine atom, a methyl group, or a trifluoromethyl group;
X³ is a single bond, *—C(═O)—O—, or *—C(═O)—NH—, and “*” represents a bond to a carbon atom of a main chain;
X⁴ is a single bond, an aliphatic hydrocarbylene group having 1 to 4 carbon atoms, a carbonyl group, a sulfonyl group, or a group obtained by combining these groups;
X⁵ and X⁶ are each independently an oxygen atom, or a sulfur atom, provided that X⁴ and X⁶ are bonded to adjacent carbon atoms of an aromatic ring;
R²² and R²³ are each independently a hydrogen atom or a hydrocarbyl group having 1 to 20 carbon atoms and optionally comprising a heteroatom, or R²² and R²³ are optionally bonded to each other to form a ring together with a carbon atom to which the R²² and R²³ are bonded; and
R²⁴ is a halogen atom, a hydroxy group, a cyano group, a nitro group, a hydrocarbyl group having 1 to 20 carbon atoms and optionally comprising a heteroatom, a hydrocarbyloxy group having 1 to 20 carbon atoms and optionally comprising a heteroatom, a hydrocarbyloxycarbonyl group having 2 to 20 carbon atoms and optionally comprising a heteroatom, a hydrocarbylthio group having 1 to 20 carbon atoms and optionally comprising a heteroatom, or —N(R^{24A})(R^{24B}) where R^{24A} and R^{24B} are each independently a hydrogen atom or a hydrocarbyl group having 1 to 6 carbon atoms, and when “b2” is 2 or more, a plurality of R²⁴ are optionally bonded to each other to form a ring together with carbon atoms of an aromatic ring to which the R²⁴ are bonded.

The chemically amplified resist composition of the present invention may contain such a base polymer.

The base polymer preferably further contains a repeating unit represented by either or both of general formulae (b1) and (b2):

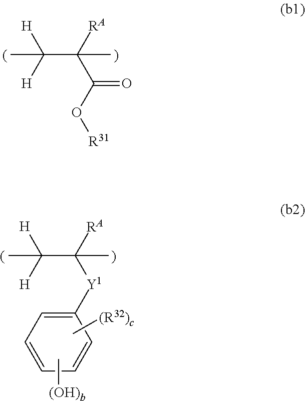

wherein each R^{A} is independently a hydrogen atom, a fluorine atom, a methyl group, or a trifluoromethyl group;
Y¹ is a single bond or *—C(═O)—O—, and represents a bond to a carbon atom of a main chain;
R³¹ is a hydrogen atom or a group having 1 to 20 carbon atoms and comprising at least one structure selected from the group consisting of a hydroxy group other than a phenolic hydroxy group, a cyano group, a carbonyl group, a carboxy group, an ether bond, an ester bond, a sulfonate ester bond, a carbonate bond, a lactone ring, a sultone ring, and a carboxylic anhydride (—C(═O)—O—C(═O)—);
R³² is a halogen atom, a carboxy group, a nitro group, a cyano group, an alkoxycarbonyl group, a hydrocarbyl group having 1 to 20 carbon atoms and optionally comprising a heteroatom, a hydrocarbyloxy group having 1 to 20 carbon atoms and optionally comprising a heteroatom, a hydrocarbylcarbonyl group having 2 to 20 carbon atoms and optionally comprising a heteroatom, a hydrocarbylcarbonyloxy group having 2 to 20 carbon atoms and optionally comprising a heteroatom, or a hydrocarbyloxycarbonyl group having 2 to 20 carbon atoms and optionally comprising a heteroatom, wherein when “c” is 2 or more, R³² are the same as or different from each other; and
“b” is an integer of 1 to 4, and “c” is an integer of 0 to 4, provided that 1≤b+c≤5.

The chemically amplified resist composition of the present invention may contain such a base polymer.

The base polymer may contain at least one selected from the group consisting of a repeating unit represented by general formula (c1), a repeating unit represented by general formula (c2), a repeating unit represented by general formula (c3), a repeating unit represented by general formula (c4), and a repeating unit represented by general formula (c5):

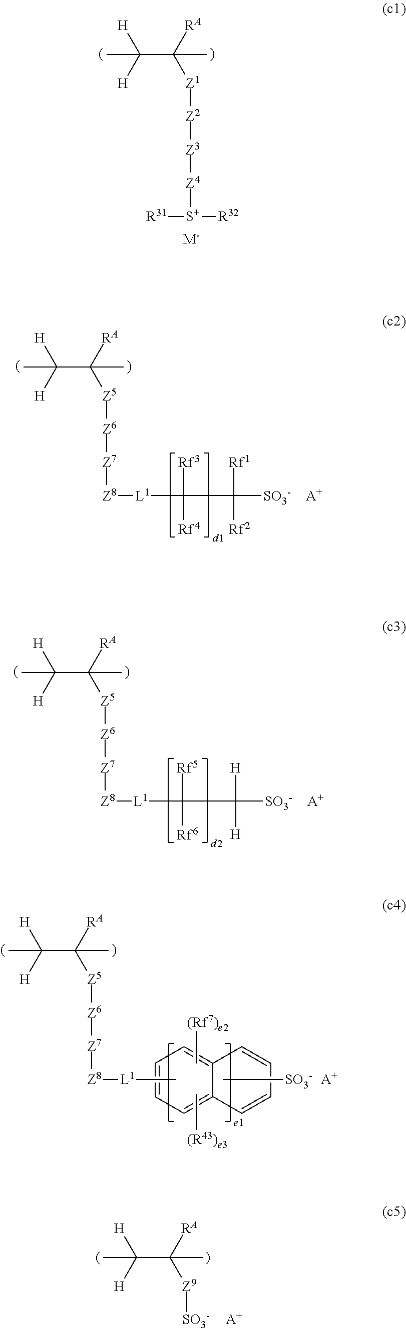

wherein “d1” and “d2” are each independently an integer of 0 to 3;
“e1” is 0 or 1, “e2” is an integer of 0 to 4, and “e3” is an integer of 0 to 4, provided that when “e1” is 0, 0≤e2+e3≤4, and when “e1” is 1, 0≤e2+e3≤6;
R^{A} is each independently a hydrogen atom, a fluorine atom, a methyl group, or a trifluoromethyl group;
Z¹ is a single bond or a phenylene group optionally having a substituent;
Z² is a single bond, **—C(═O)—O—Z²¹—, **—C(═O)—NH—Z²¹—, or **—O—Z²¹—, where Z²¹ is an aliphatic hydrocarbylene group having 1 to 6 carbon atoms, a phenylene group, or a divalent group obtained by combining these groups, and optionally comprises a halogen atom, a carbonyl group, an ester bond, an ether bond, or a hydroxy group;
Z³ is a single bond, an ether bond, an ester bond, a sulfonate ester bond, an amide bond, a sulfonamide bond, a carbonate bond, or a carbamate bond;
Z⁴ is a single bond, an aliphatic hydrocarbylene group having 1 to 6 carbon atoms, a phenylene group, or a divalent group obtained by combining these groups, and optionally comprises a halogen atom, a carbonyl group, an ester bond, an ether bond, or a hydroxy group;
each Z⁵ is independently a single bond, a phenylene group or naphthylene group optionally having a substituent, or *—C(═O)—O—Z⁵¹—, where Z⁵¹ is an aliphatic hydrocarbylene group having 1 to 10 carbon atoms, a phenylene group, or a naphthylene group, wherein the aliphatic hydrocarbylene group optionally comprises a halogen atom, a hydroxy group, an ether bond, an ester bond, or a lactone ring;
Z⁶ is a single bond, an ether bond, an ester bond, a sulfonate ester bond, an amide bond, a sulfonamide bond, a carbonate bond, or a carbamate bond;
each Z⁷ is independently a single bond, ***—Z⁷¹—C(═O)—O—, ***—C(═O)—NH—Z⁷¹—, or ***—O—Z⁷¹—, where Z⁷¹ is a hydrocarbylene group having 1 to 20 carbon atoms and optionally comprising a heteroatom;
each Z⁸ is independently a single bond, ****—Z⁸¹—C(═O)—O—, ****—C(═O)—NH—Z⁸¹—, or ****—O—Z⁸¹—, where Z⁸¹ is a hydrocarbylene group having 1 to 20 carbon atoms and optionally comprising a heteroatom;
Z⁹ is a single bond, a methylene group, an ethylene group, a phenylene group, a fluorinated phenylene group, a phenylene group substituted with a trifluoromethyl group, *—C(═O)—O—Z⁹¹—, *—C(═O)—NH—Z⁹¹—, or *—O—Z⁹¹—, where Z⁹¹ is an aliphatic hydrocarbylene group having 1 to 6 carbon atoms, a phenylene group, a fluorinated phenylene group, or a phenylene group substituted with a trifluoromethyl group, and optionally comprises a carbonyl group, an ester bond, an ether bond, or a hydroxy group;
“*” represents a bond to a carbon atom of a main chain, “**” represents a bond to Z¹, and “***” represents a bond to Z⁶, and “****” represents a bond to Z⁷;
L¹ is a single bond, an ether bond, an ester bond, a carbonyl group, a sulfonate ester bond, a sulfonamide bond, a carbonate bond, or a carbamate bond;
Rf¹ and Rf² are each independently a fluorine atom or a fluorinated saturated hydrocarbyl group having 1 to 6 carbon atoms;
Rf³ and Rf⁴ are each independently a hydrogen atom, a fluorine atom, or a fluorinated saturated hydrocarbyl group having 1 to 6 carbon atoms;
Rf⁵ and Rf⁶ are each independently a hydrogen atom, a fluorine atom, or a fluorinated saturated hydrocarbyl group having 1 to 6 carbon atoms, provided that not all Rf⁵ and Rf⁶ are hydrogen atoms at the same time;
RF⁷ is a fluorine atom, a fluorinated alkyl group having 1 to 6 carbon atoms, a fluorinated alkoxy group having 1 to 6 carbon atoms, or a fluorinated alkylthio group having 1 to 6 carbon atoms;
R⁴¹ and R⁴² are each independently a hydrocarbyl group having 1 to 20 carbon atoms and optionally comprising a heteroatom, or R⁴¹ and R⁴² are optionally bonded to each other to form a ring together with a sulfur atom to which the R⁴¹ and R⁴² are bonded;
R⁴³ is a halogen atom other than a fluorine atom or a hydrocarbyl group having 1 to 20 carbon atoms and optionally comprising a heteroatom, wherein when “e3” is 2, 3, or 4, a plurality of R⁴³ are optionally bonded to each other to form a ring together with carbon atoms to which the R⁴³ are bonded;
M⁻ is a non-nucleophilic counter ion; and
A⁺ is an onium cation.

The chemically amplified resist composition of the present invention may contain such a base polymer.

The chemically amplified resist composition may further contain at least one selected from the group consisting of an organic solvent, a quencher, a photoacid generator other than the photoacid generator, and surfactant.

The chemically amplified resist composition of the present invention can contain such components.

Furthermore, the present invention provides a patterning process comprising: forming a resist film on a substrate by using the chemically amplified resist composition of the present invention; exposing the resist film to high-energy radiation; and developing the exposed resist film by using a developing solution.

When patterning is performed using such a patterning process, the chemically amplified resist composition exhibits high sensitivity, excellent acid diffusion suppression, and superior lithography performance, making it effective for fine patterning.

The patterning process can be performed using KrF excimer laser, ArF excimer laser, electron beam, or extreme ultraviolet radiation having a wavelength of 3 to 15 nm as the high-energy radiation.

Such high-energy radiation can be used in the patterning process of the present invention.

### Advantageous Effects of Invention

When patterning is performed using a chemically amplified resist composition containing the onium salt of the present invention as a photoacid generator, the chemically amplified resist composition has high sensitivity, excellent ability to suppress acid diffusion, improved lithography performance such as LWR, CDU, MEF, EL, and DOF, thereby suppressing the collapse of the resist pattern during fine patterning.

### DESCRIPTION OF EMBODIMENTS

As described above, development of a resist material useful for further fine patterning has been required for the performance of the resist material.

As a result of intensive studies on the above problems, the present inventor has found that an onium salt composed of an aromatic sulfonate anion substituted with a hydrocarbyl group containing at least one aromatic ring and a sulfonium cation containing a nitro group has excellent solvent solubility, and a chemically amplified resist composition using the onium salt as a photoacid generator has high sensitivity and high contrast, is extremely effective in suppressing acid diffusion, is excellent in lithography performance such as LWR, CDU, MEF, EL, and DOF, and is extremely effective in fine patterning, and thus completed the present invention.

In other words, the present invention is an onium salt comprising an anion represented by general formula (1A) and a cation represented by general formula (1B):

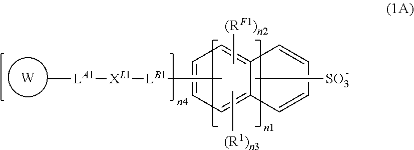

wherein “n1” is 0 or 1, “n2” is an integer of 0 to 4, “n3” is an integer of 0 to 4, provided that when “n1” is 0, 0≤n2+n3≤4, and when “n1” is 1, 0≤n2+n3≤6, and “n4” is 0 or 1;
W is a hydrocarbyl group having 6 to 60 carbon atoms and comprising at least one aromatic ring, wherein the hydrocarbyl group optionally comprises a heteroatom;
R^{F1} is a fluorine atom, a fluorinated saturated hydrocarbyl group having 1 to 6 carbon atoms, a fluorinated saturated hydrocarbyloxy group having 1 to 6 carbon atoms, or a fluorinated saturated hydrocarbylthio group having 1 to 6 carbon atoms;
R¹ is a halogen atom other than fluorine, a nitro group, a hydrocarbyl group having 1 to 20 carbon atoms and optionally comprising a heteroatom, a hydrocarbyloxy group having 1 to 20 carbon atoms and optionally comprising a heteroatom, or a hydrocarbylthio group having 1 to 20 carbon atoms and optionally comprising a heteroatom, wherein when “n3” is 2, 3, or 4, R¹ are the same as or different from each other, and a plurality of R¹ are optionally bonded to each other to form a ring together with carbon atoms to which the R¹ are bonded;
L^{A1} and L^{B1} are each independently a single bond, an ether bond, an ester bond, a sulfonate ester bond, an amide bond, a sulfonamide bond, a carbonate bond, or a carbamate bond;
X^{L1} is a single bond or a hydrocarbylene group having 1 to 40 carbon atoms and optionally comprising a heteroatom, and

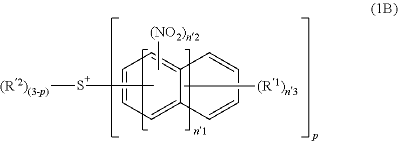

wherein “p” is 1, 2, or 3, “n′¹” is 0 or 1, “n′²” is 1 or 2, and “n′³” is an integer of 0 to 6, provided that when “n′¹” is 0, 1≤n′²+n′3≤5, and when “n′1” is 1, 1≤n′2+n′3≤7;
R′¹ is a halogen atom, a cyano group, a hydroxy group, a carboxy group, an alkoxycarbonyl group, a hydrocarbyl group having 1 to 20 carbon atoms and optionally comprising a heteroatom, a hydrocarbyloxy group having 1 to 20 carbon atoms and optionally comprising a heteroatom, or a hydrocarbylthio group having 1 to 20 carbon atoms and optionally comprising a heteroatom, wherein when “n′3” is 2 or 3, R′¹ are the same as or different from each other, and two R′¹ are optionally bonded to each other to form a ring together with carbon atoms to which the R′¹ are bonded; and
R′² is a halogen atom or a hydrocarbyl group having 1 to 30 carbon atoms and optionally comprising a heteroatom, wherein when “p” is 1, two R′² are the same as or different from each other, and two of three substituents bonded to S⁺ are optionally bonded to each other to form a ring together with a sulfur atom to which the substituents are bonded.

Hereinafter, the present invention will be described in detail. However, the present invention is not limited thereto.

Note that for some structures represented by chemical formulae, there can exist enantiomers and diastereomers because of the presence of asymmetric carbon atoms. In such a case, a single formula collectively represents all such isomers. The isomers may be used alone or in admixture of two or more thereof.

### Onium Salt

An onium salt of the present invention contains an anion represented by formula (1A):

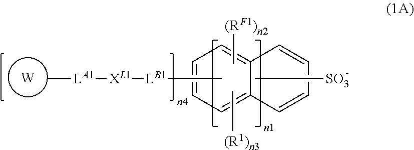

wherein “n1” is 0 or 1, “n2” is an integer of 0 to 4, “n3” is an integer of 0 to 4, provided that when “n1” is 0, 0≤n2+n3≤4, and when “n1” is 1, 0≤n2+n3≤6, and “n4” is 0 or 1;
W is a hydrocarbyl group having 6 to 60 carbon atoms and comprising at least one aromatic ring, wherein the hydrocarbyl group optionally comprises a heteroatom;
R^{F1} is a fluorine atom, a fluorinated saturated hydrocarbyl group having 1 to 6 carbon atoms, a fluorinated saturated hydrocarbyloxy group having 1 to 6 carbon atoms, or a fluorinated saturated hydrocarbylthio group having 1 to 6 carbon atoms;
R¹ is a halogen atom other than fluorine, a nitro group, a hydrocarbyl group having 1 to 20 carbon atoms and optionally comprising a heteroatom, a hydrocarbyloxy group having 1 to 20 carbon atoms and optionally comprising a heteroatom, or a hydrocarbylthio group having 1 to 20 carbon atoms and optionally comprising a heteroatom, wherein when “n3” is 2, 3, or 4, R¹ are the same as or different from each other, and a plurality of R¹ are optionally bonded to each other to form a ring together with carbon atoms to which the R¹ are bonded;
L^{A1} and L^{B1} are each independently a single bond, an ether bond, an ester bond, a sulfonate ester bond, an amide bond, a sulfonamide bond, a carbonate bond, or a carbamate bond; and
X^{L1} is a single bond or a hydrocarbylene group having 1 to 40 carbon atoms and optionally comprising a heteroatom.

In formula (1A), “n1” is 0 or 1. The onium salt has a benzene ring when “n1” is 0, and a naphthalene ring when “n1” is 1. From the viewpoint of solvent solubility, a benzene ring with “n1” of 0 is preferable. “n2” is an integer of 0 to 4. From the viewpoint of raw material procurement, “n2” is preferably 4 when “n2” is 1 or more. “n3” is an integer of 0 to 4. However, when “n1” is 0, 0≤n2+n3≤4, and when “n1” is 1, 0≤n2+n3≤6. “n4” is 0 or 1, and from the viewpoint of acid diffusion control, “n4” is preferably 1.

In formula (1A), R^{F1} is a fluorine atom, a fluorinated saturated hydrocarbyl group having 1 to 6 carbon atoms, a fluorinated saturated hydrocarbyloxy group having 1 to 6 carbon atoms, or a fluorinated saturated hydrocarbylthio group having 1 to 6 carbon atoms. R^{F1} is preferably a fluorine atom, a trifluoromethyl group, a difluoromethyl group, a trifluoromethoxy group, a difluoromethoxy group, a trifluoromethylthio group, or a difluoromethylthio group, and more preferably a fluorine atom, a trifluoromethyl group, or a trifluoromethoxy group. When a fluorine atom or a substituent having the fluorine atom is contained, the generated acid has so high acid strength due to the electron withdrawing effect that deprotection reaction of acid labile groups such as a tertiary ester and a tertiary ether takes place smoothly. When “n2” is 2, 3, or 4, R^{F1} may be the same as or different from each other.

In formula (1A), R¹ is a halogen atom other than fluorine, a nitro group, a hydrocarbyl group having 1 to 20 carbon atoms and optionally comprising a heteroatom, a hydrocarbyloxy group having 1 to 20 carbon atoms and optionally comprising a heteroatom, or a hydrocarbylthio group having 1 to 20 carbon atoms and optionally comprising a heteroatom. Examples of the halogen atom other than the fluorine atom include a chlorine atom, a bromine atom, and an iodine atom, and the halogen atom is preferably an iodine atom. The hydrocarbyl group may be either saturated or unsaturated and linear, branched, or cyclic. Specific examples thereof include alkyl groups having 1 to 20 carbon atoms, such as a methyl group, an ethyl group, an n-propyl group, an isopropyl group, an n-butyl group, an isobutyl group, a sec-butyl group, a tert-butyl group, an n-pentyl group, an n-hexyl group, an n-octyl group, an n-nonyl group, an n-decyl group, an undecyl group, a dodecyl group, a tridecyl group, a tetradecyl group, a pentadecyl group, a heptadecyl group, an octadecyl group, a nonadecyl group, and an icosyl group; cyclic saturated hydrocarbyl groups having 3 to 20 carbon atoms, such as a cyclopropyl group, a cyclopentyl group, a cyclohexyl group, a cyclopropylmethyl group, a 4-methylcyclohexyl group, a cyclohexylmethyl group, a norbornyl group, and an adamantyl group; alkenyl groups having 2 to 20 carbon atoms, such as a vinyl group, an allyl group, a propenyl group, a butenyl group, and a hexenyl group; cyclic unsaturated hydrocarbyl groups having 3 to 20 carbon atoms, such as a cyclohexenyl group; aryl groups having 6 to 20 carbon atoms, such as a phenyl group or a naphthyl group; aralkyl groups having 7 to 20 carbon atoms, such as a benzyl group, a 1-phenylethyl group, and a 2-phenylethyl group; and groups obtained by combining these groups. Of these, preferred is an aryl group. Some or all of the hydrogen atoms in the hydrocarbyl group are optionally substituted with a group containing a heteroatom such as an oxygen atom, a sulfur atom, a nitrogen atom, or a halogen atom, and some of —CH₂— in the hydrocarbyl group are optionally substituted with a group containing a heteroatom such as an oxygen atom, a sulfur atom, or a nitrogen atom. As a result, the hydrocarbyl group may contain, for instance, a hydroxy group, a cyano group, a fluorine atom, a chlorine atom, a bromine atom, an iodine atom, a carbonyl group, an ether bond, an ester bond, a sulfonate ester bond, a carbonate bond, a lactone ring, a sultone ring, a carboxylic anhydride (—C(═O)—O—C(═O)—), and a haloalkyl group. When “n3” is 2, 3, or 4, R¹ are the same as or different from each other. In addition, when “n3” is 2, 3, or 4, a plurality of R¹ are optionally bonded to each other to form a ring together with carbon atoms to which the R¹ are bonded. The ring is preferably a 5- to 8-membered ring.

In formula (1A), W is a hydrocarbyl group having 6 to 60 carbon atoms, preferably 6 to 40 carbon atoms, and comprising at least one aromatic ring, wherein the hydrocarbyl group optionally comprises a heteroatom.

In formula (1A), the W is preferably represented by a group of general formula (W-1) or (W-2):

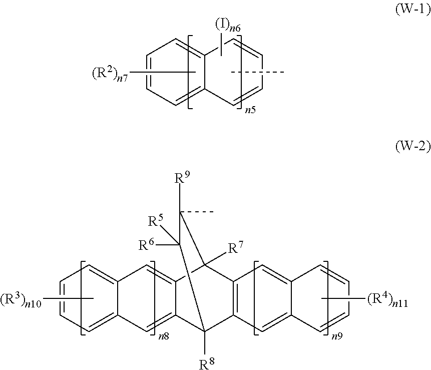

wherein “n5” is 0 or 1, “n6” is an integer of 0 to 4, “n7” is an integer of 1 to 4, provided that when “n5” is 0, 1≤n6+n7≤5, and when “n5” is 1, 1≤n6+n7≤7, “n8” is 0 or 1, “n9” is 0 or 1, “n10” is an integer of 0 to 4, and “n11” is an integer of 0 to 4;
each R² is independently a hydrogen atom, a halogen atom other than an iodine atom, or a hydrocarbyl group having 1 to 20 carbon atoms and optionally comprising a heteroatom;
R³ and R⁴ are each independently a hydrogen atom, a halogen atom, or a hydrocarbyl group having 1 to 20 carbon atoms and optionally comprising a heteroatom;
R⁵ to R⁹ are each independently a hydrogen atom, a halogen atom, or a hydrocarbyl group having 1 to 20 carbon atoms and optionally comprising a heteroatom; and
the dashed line represents a bond to L^{A1}.

In formula (W-1), “n5” is 0 or 1. The onium salt has a benzene ring when “n5” is 0, and a naphthalene ring when “n5” is 1. From the viewpoint of solvent solubility, a benzene ring with “n5” of 0 is preferable. “n6” is an integer of 0 to 4. When “n5” is 0, “n6” is preferably 2, 3, or 4. From the viewpoint of EUV light absorption, “n6” is preferably 3 or 4. “n7” is an integer of 1 to 4. From the viewpoint of raw material procurement, “n7” is preferably 1 to 3. From the viewpoint of acid diffusion control, “n7” is preferably 2 or 3.

In formula (W-1), each R² is independently a hydrogen atom, a halogen atom other than an iodine atom, or a hydrocarbyl group having 1 to 20 carbon atoms and optionally comprising a heteroatom. Examples of the halogen atom other than the iodine atom include a fluorine atom, a chlorine atom, and a bromine atom. From the viewpoint of solvent solubility, the halogen atom is preferably a fluorine atom. Specific examples of the hydrocarbyl group having 1 to 20 carbon atoms and optionally comprising a heteroatom include, but are not limited to, the same hydrocarbyl group represented by R¹ in the description of general formula (1A). R² is preferably a group having a branched structure or a cyclic structure. Note that the maximum number of carbon atoms in general formula (W-1) above is 60.

In formula (W-1), at least one selected from the group consisting of R² and an iodine atom is preferably bonded to a carbon atom bonded to a carbon atom adjacent to a carbon atom to which L^{A1} is bonded. As a result, the rotation between an aromatic ring to which these groups are bonded and an aromatic ring to which the sulfo group is bonded around the -L^{A1}-X^{L1}-L^{B1}- bond axis is suppressed due to steric hindrance, thereby reducing acid diffusion.

In formula (W-2), “n8” is 0 or 1. The onium salt has a benzene ring when “n8” is 0, and a naphthalene ring when “n8” is 1. From the viewpoint of solvent solubility, a benzene ring with “n8” of 0 is preferable. “n9” is 0 or 1. The onium salt has a benzene ring when “n9” is 0, and a naphthalene ring when “n9” is 1. From the viewpoint of solvent solubility, a benzene ring with “n9” of 0 is preferable. “n10” is an integer of 0 to 4. From the viewpoint of raw material procurement, “n10” is preferably 0, 1, or 2. “n11” is an integer of 0 to 4. From the viewpoint of raw material procurement, “n11” is preferably 0, 1, or 2.

In formula (W-2), R³ and R⁴ are each independently a hydrogen atom, a halogen atom, or a hydrocarbyl group having 1 to 20 carbon atoms and optionally comprising a heteroatom. Examples of the halogen atom include a fluorine atom, a chlorine atom, a bromine atom, and an iodine atom, and the halogen atom is preferably a fluorine atom or an iodine atom. Specific examples of the hydrocarbyl group having 1 to 20 carbon atoms and optionally comprising a heteroatom include, but are not limited to, the same hydrocarbyl group represented by R¹ in the description of general formula (1A). R³ and R⁴ are preferably groups having a branched structure or a cyclic structure.

In formula (W-2), R⁵ to R⁹ are each independently a hydrogen atom, a halogen atom, or a hydrocarbyl group having 1 to 20 carbon atoms and optionally comprising a heteroatom. Examples of the halogen atom include a fluorine atom, a chlorine atom, a bromine atom, and an iodine atom, and the halogen atom is preferably a fluorine atom or an iodine atom. Specific examples of the hydrocarbyl group having 1 to 20 carbon atoms and optionally comprising a heteroatom include, but are not limited to, the same hydrocarbyl group represented by R¹ in the description of general formula (1A). R⁵ to R⁹ are preferably groups having a branched structure or a cyclic structure. Note that the maximum number of carbon atoms in general formula (W-2) above is 60.

In formula (W-2), any two of R⁵ to R⁹ are optionally bonded to each other to form a ring together with a carbon atom to which these are bonded. The ring is preferably a 5- to 8-membered ring.

In formula (1A), L^{A1} and L^{B1} are each independently a single bond, an ether bond, an ester bond, a sulfonate ester bond, an amide bond, a sulfonamide bond, a carbonate bond, or a carbamate bond. Of these, a single bond, an ether bond, or an ester bond is preferable.

In formula (1A), X^{L1} is a single bond or a hydrocarbylene group having 1 to 40 carbon atoms and optionally comprising a heteroatom. The hydrocarbylene group may be linear, branched, or cyclic. Specific examples thereof include an alkandiyl group, a cyclic saturated hydrocarbylene group, and an arylene group. Specific examples of the heteroatom include an oxygen atom, a nitrogen atom, and a sulfur atom.

Specific examples of the hydrocarbylene group having 1 to 40 carbon atoms and optionally comprising a heteroatom represented by X^{L1} are given below, but not limited thereto. In formulae below, each * represents a bond to L^{A1} and L^{B1}.

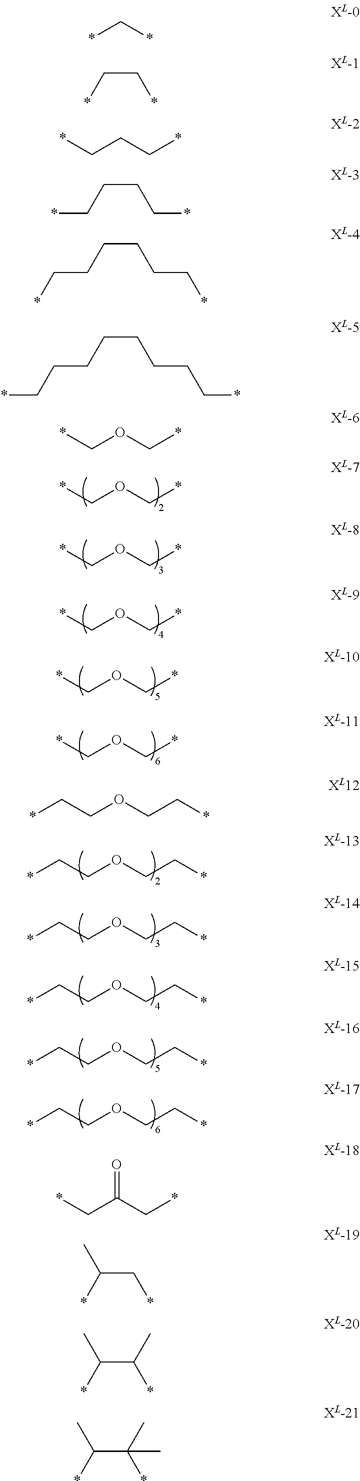

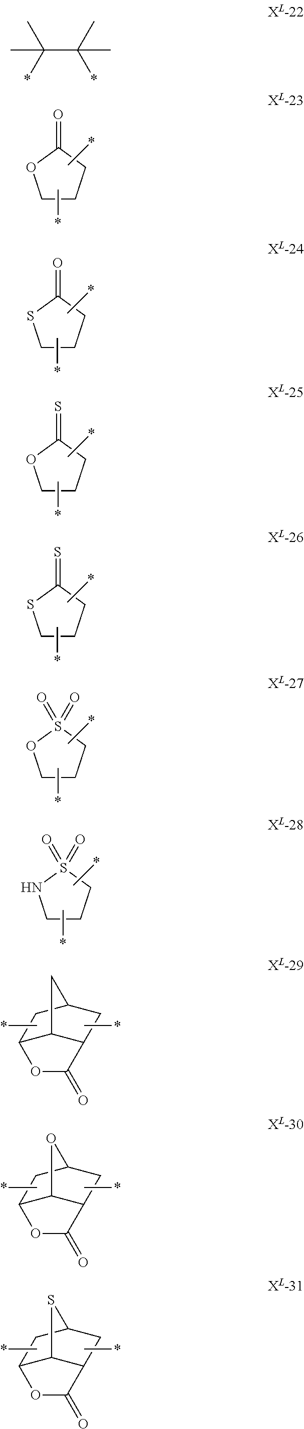

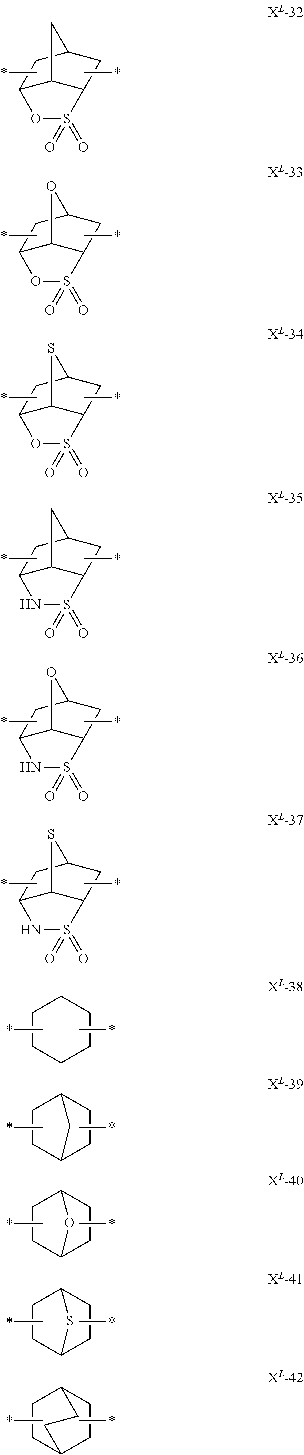

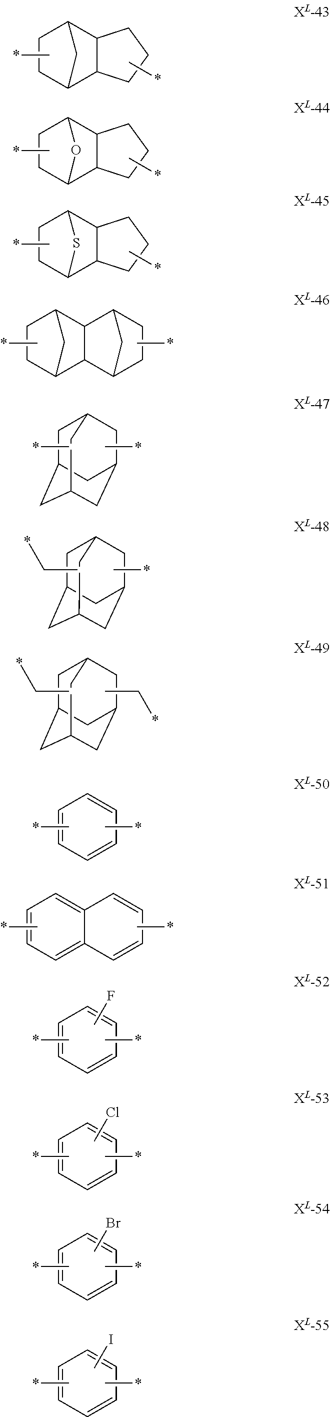

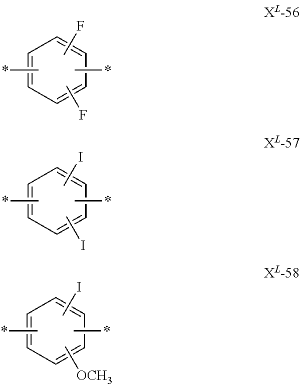

Of these, XL-0 to XL-22 and XL-47 to XL-58 are preferable.

The anion represented by formula (1A) is preferably represented by formula (1A-1):

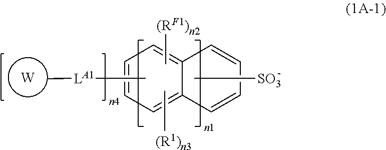

wherein “n1” to “n4”, W, R^{F1}, R¹, and L^{A1} are the same as above.

Specific examples of the anion represented by formula (1A) are given below, but not limited thereto. In formulae below, Me represents a methyl group.

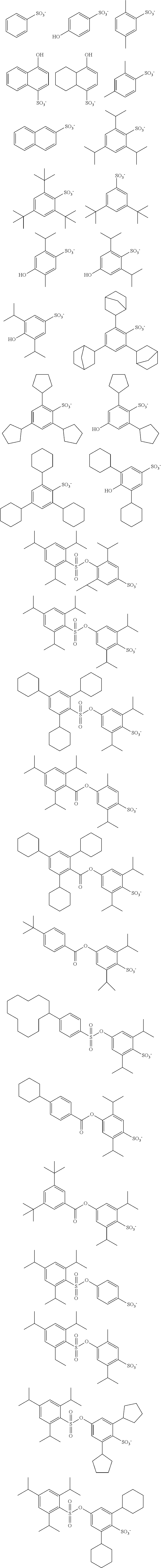

[chemical structure]

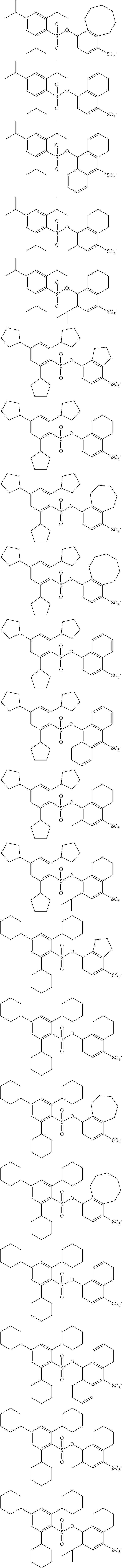

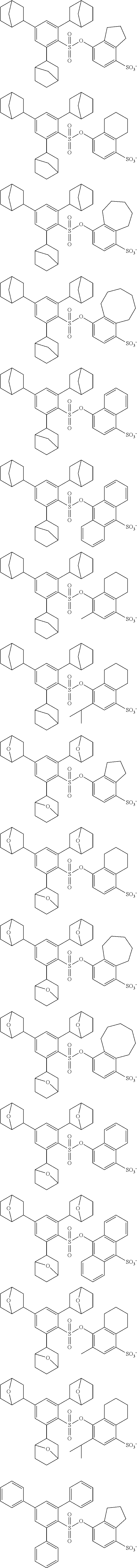

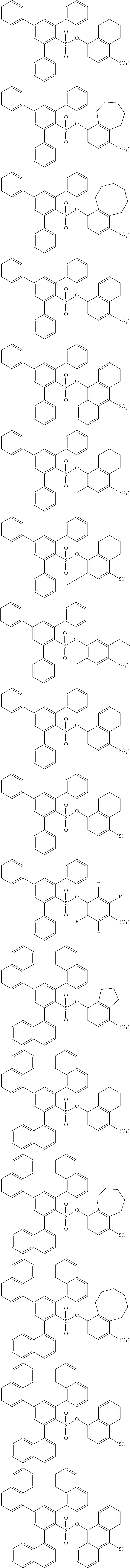

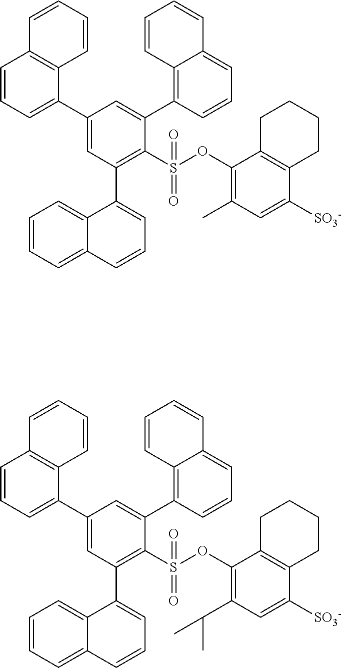

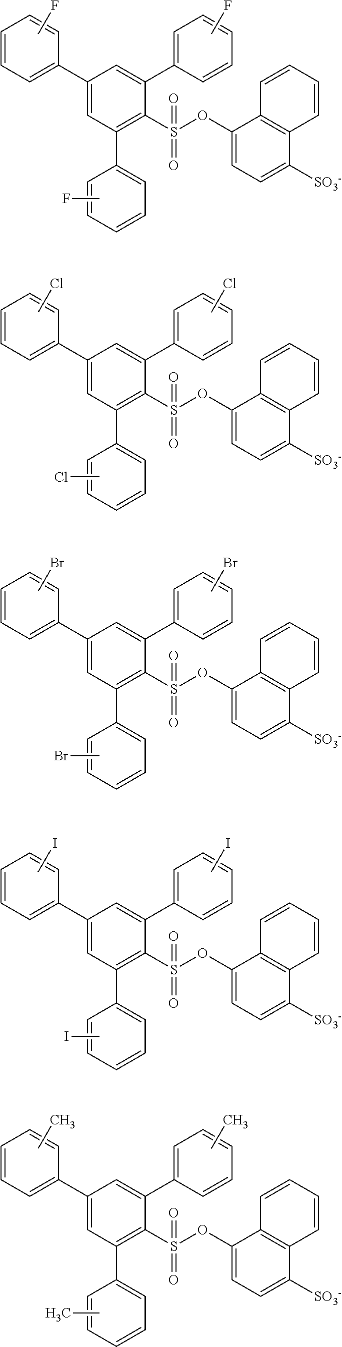

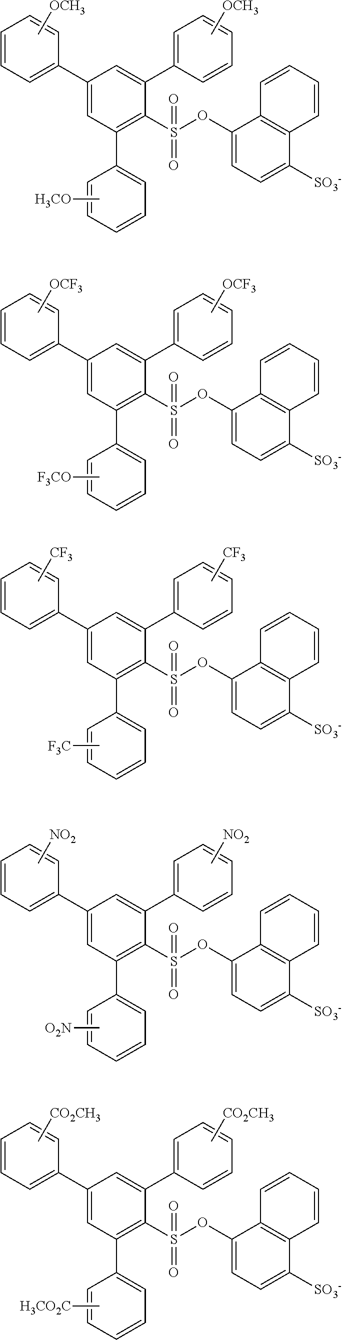

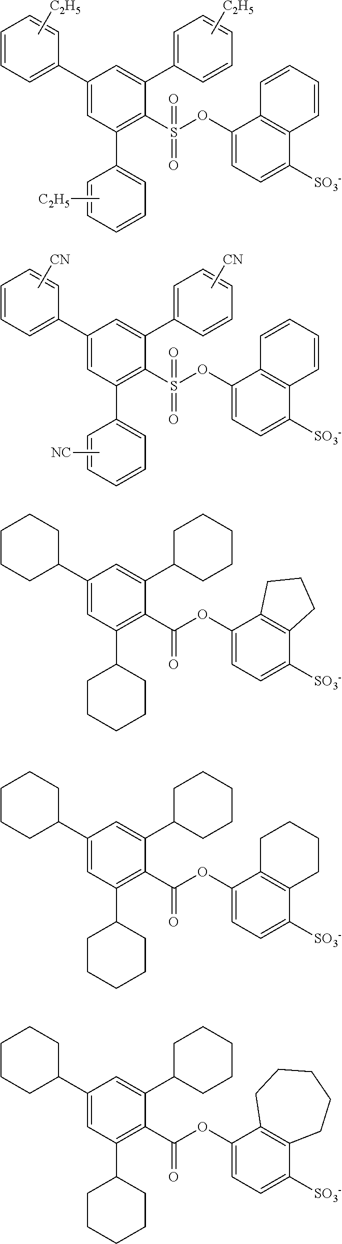

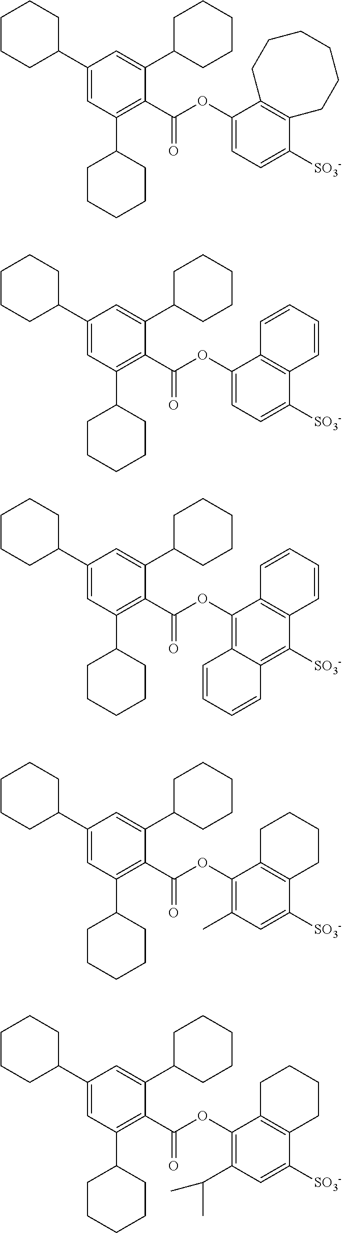

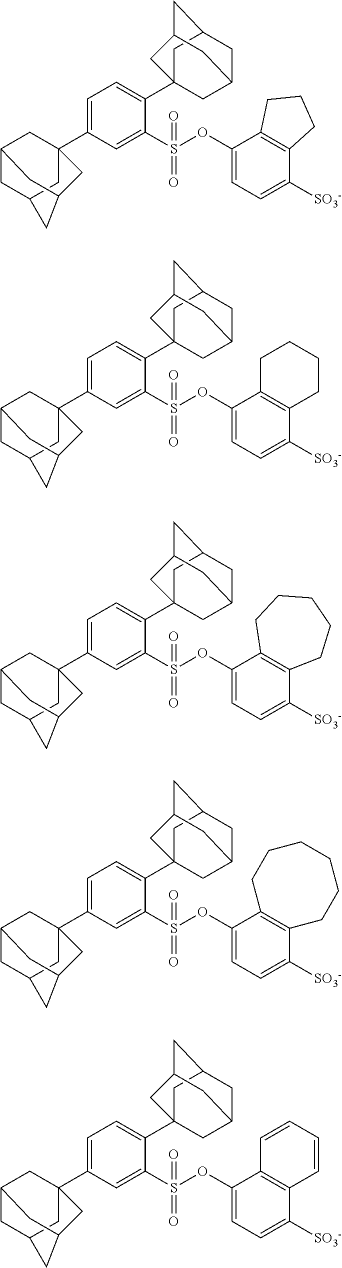

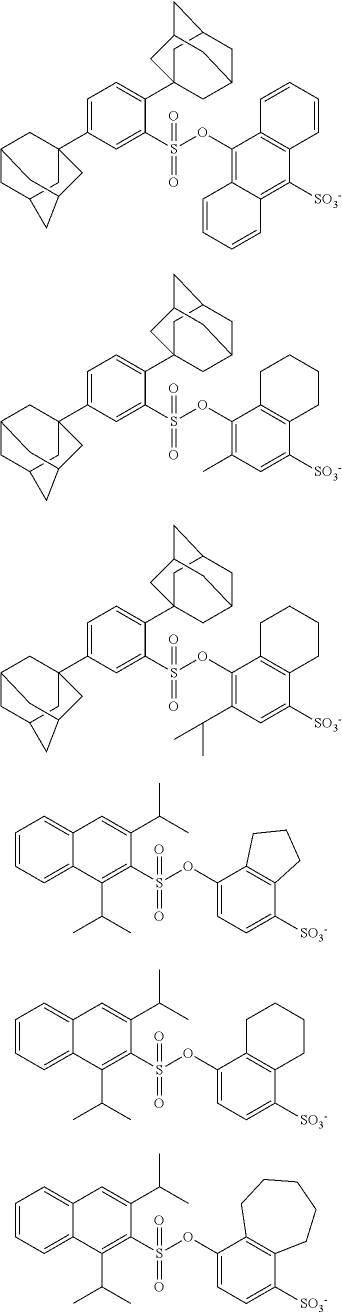

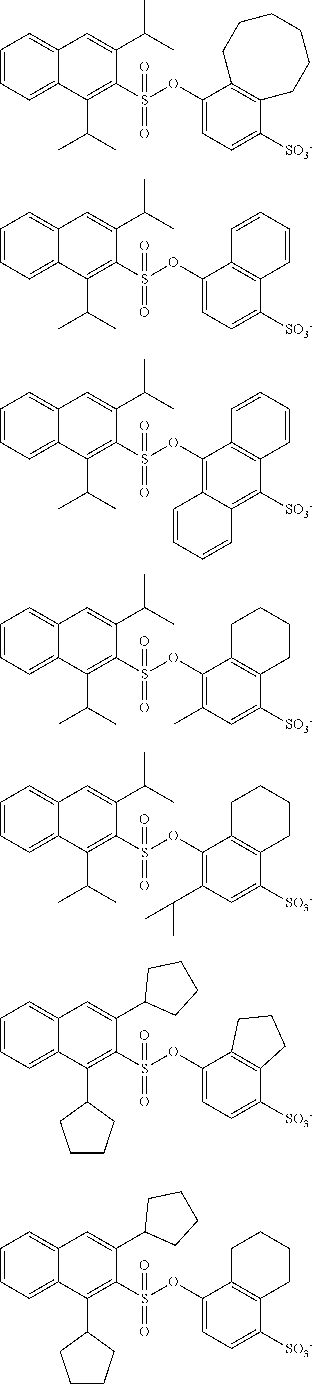

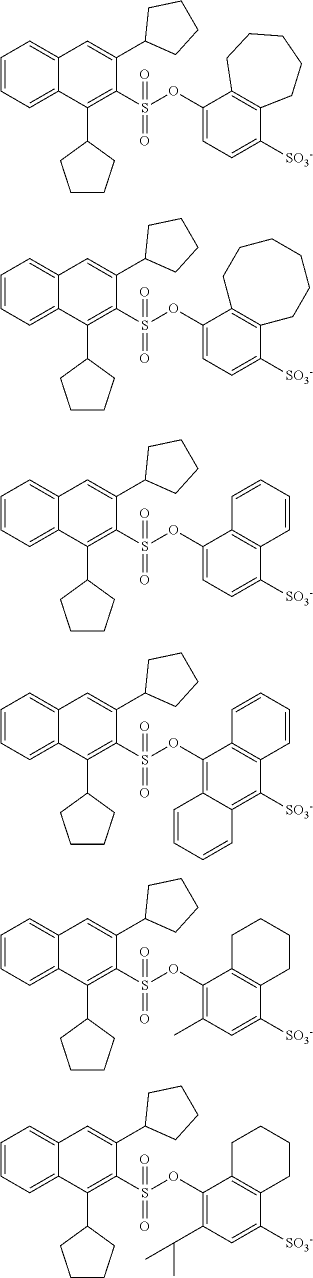

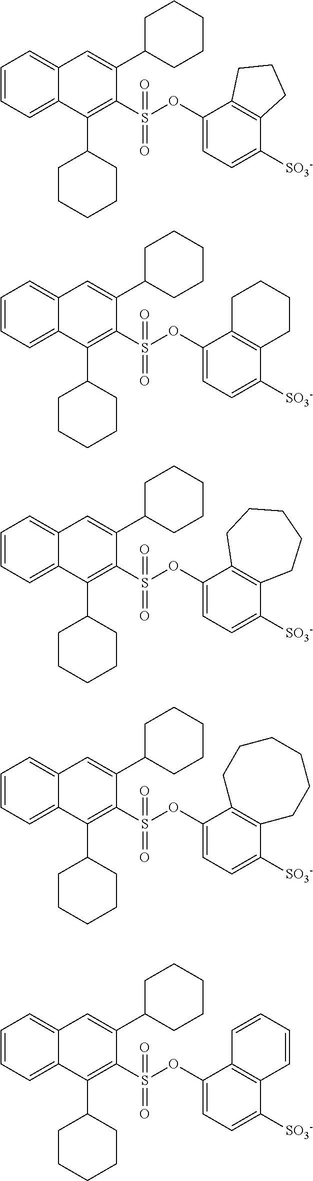

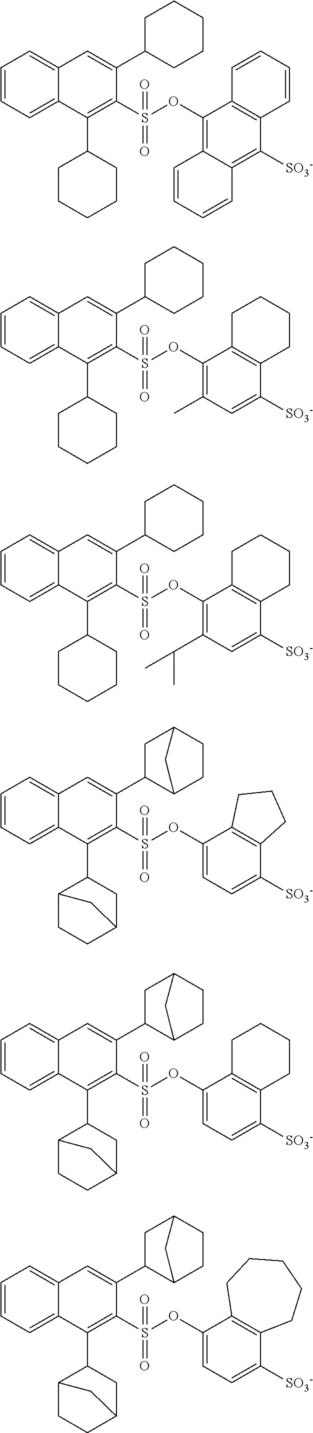

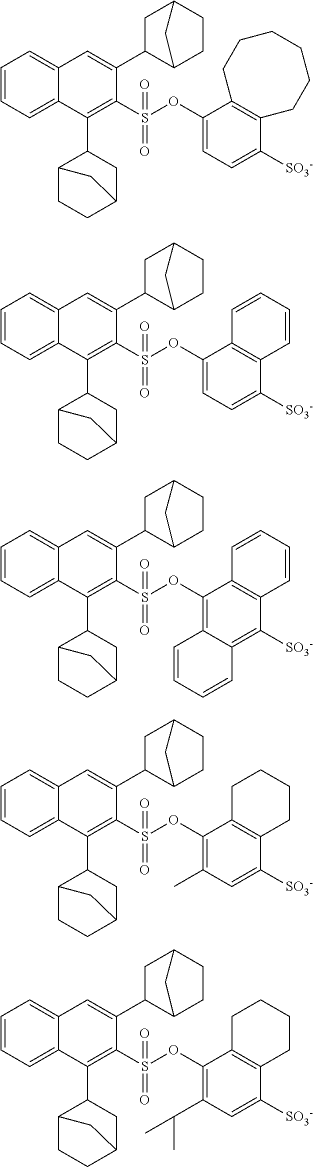

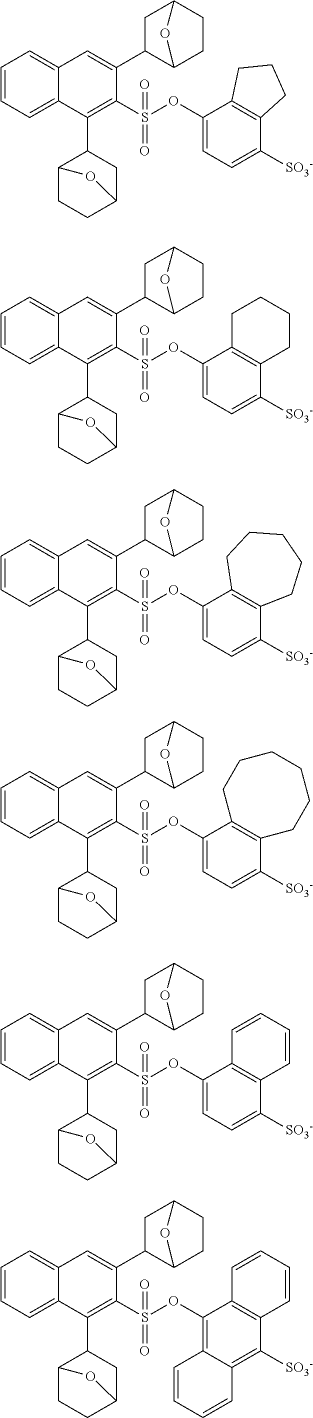

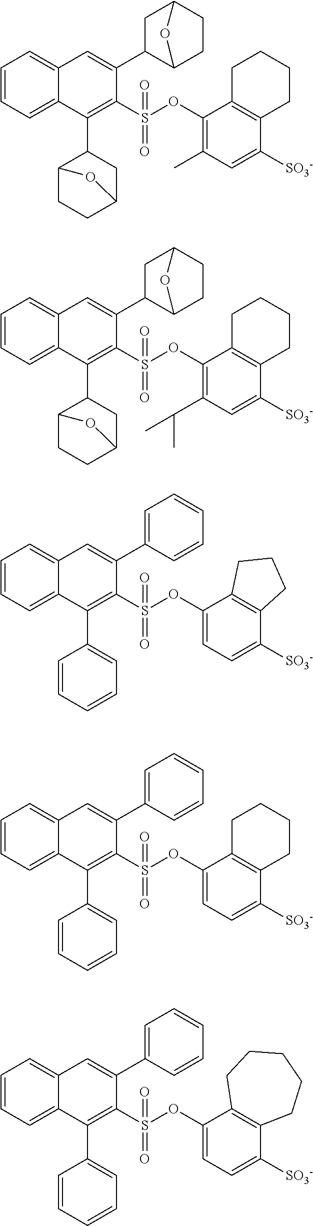

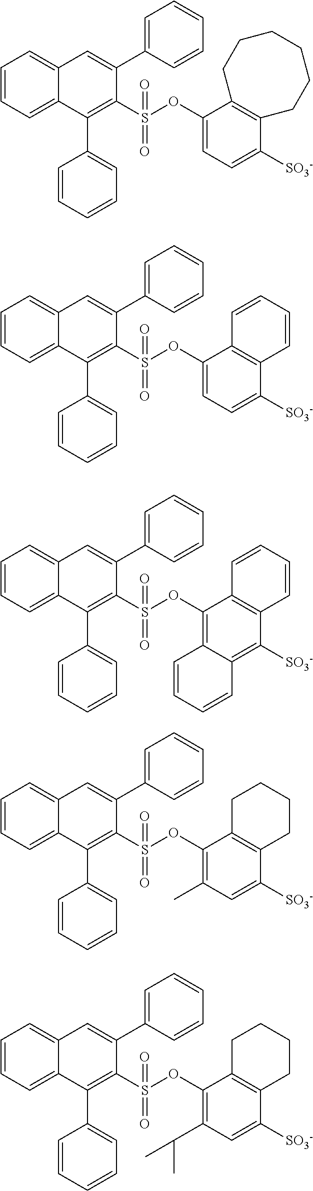

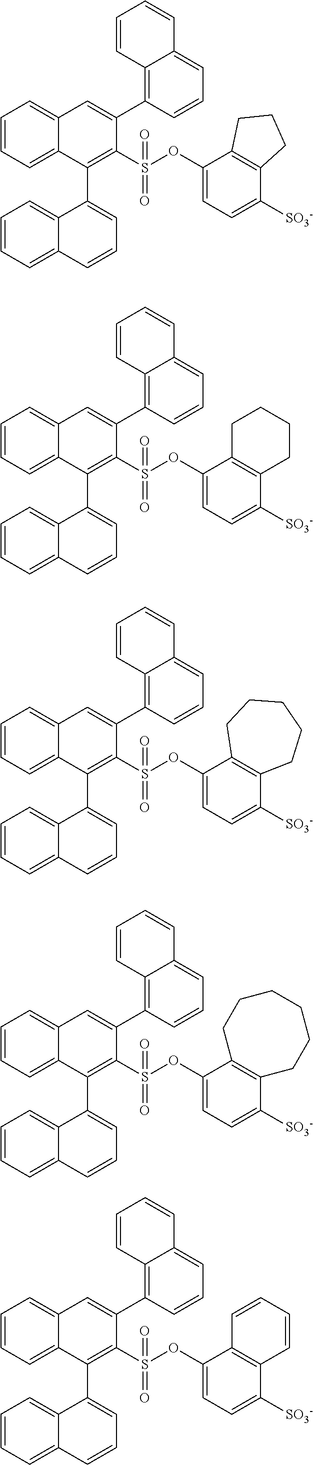

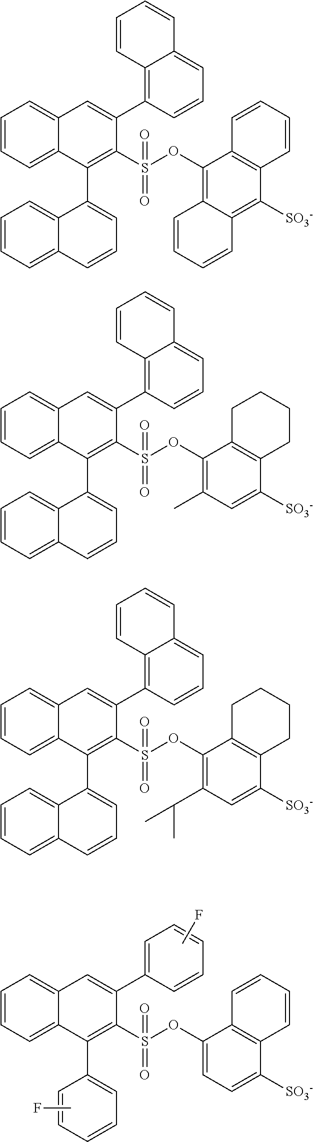

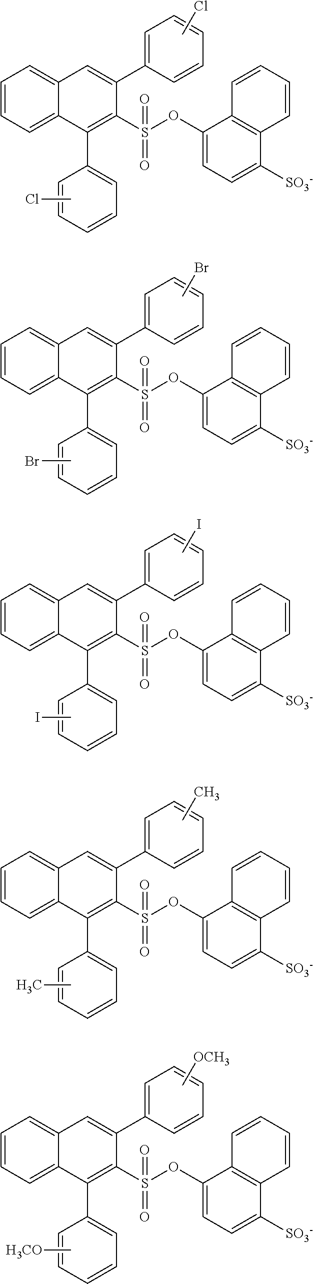

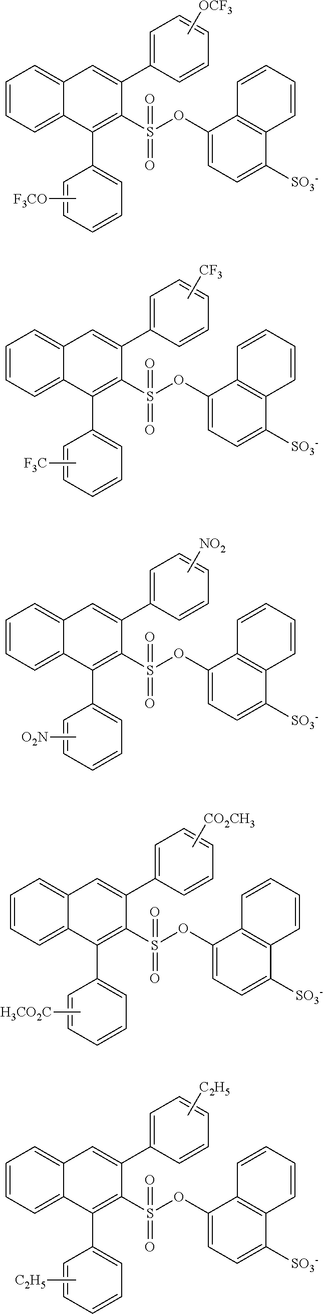

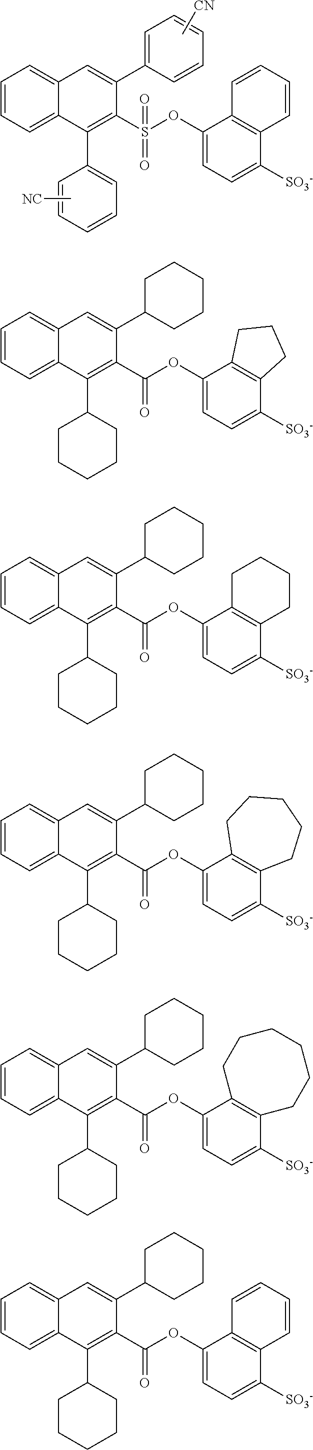

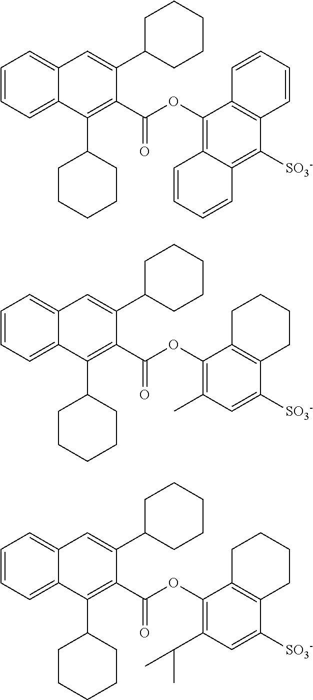

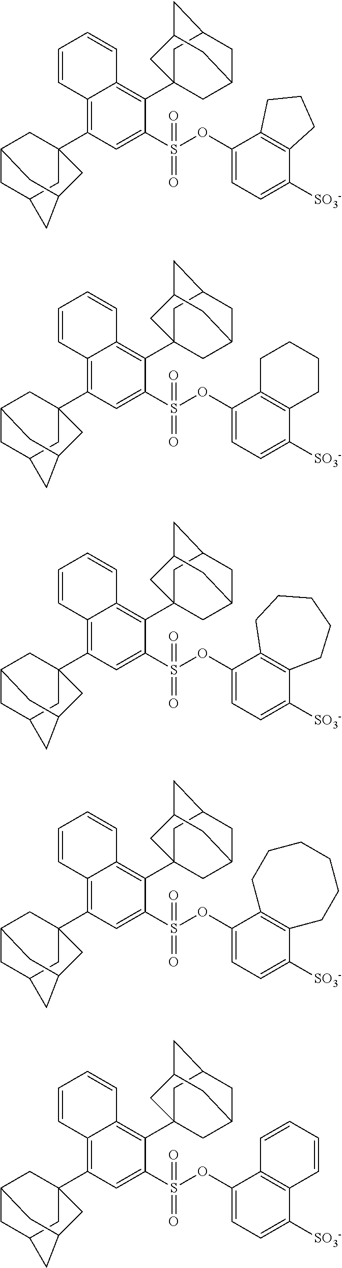

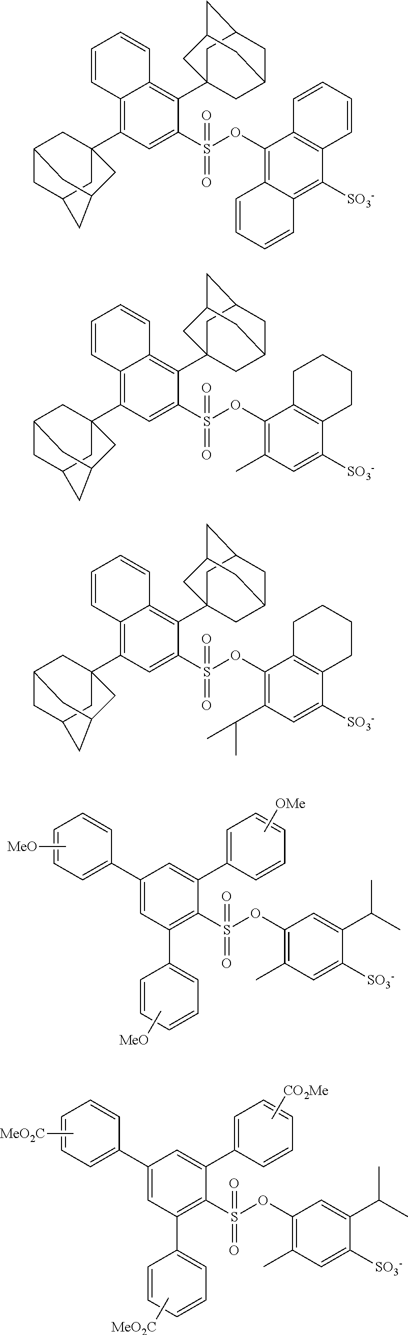

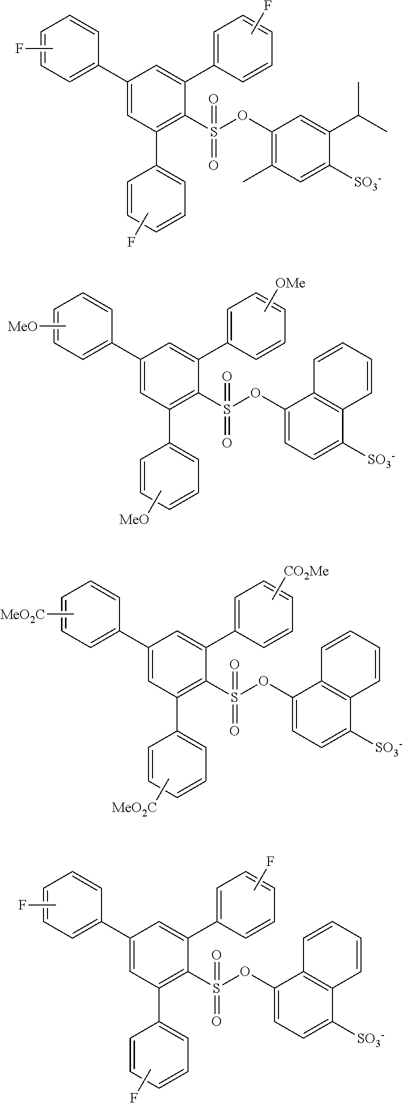

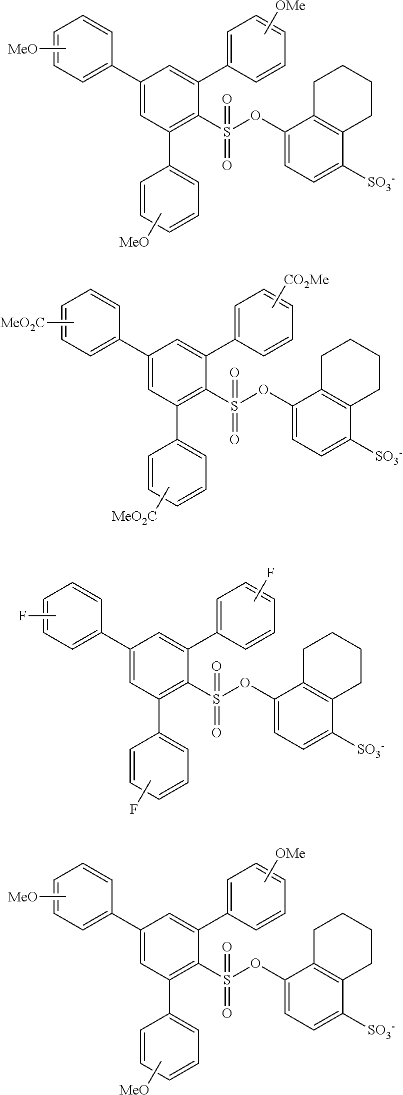

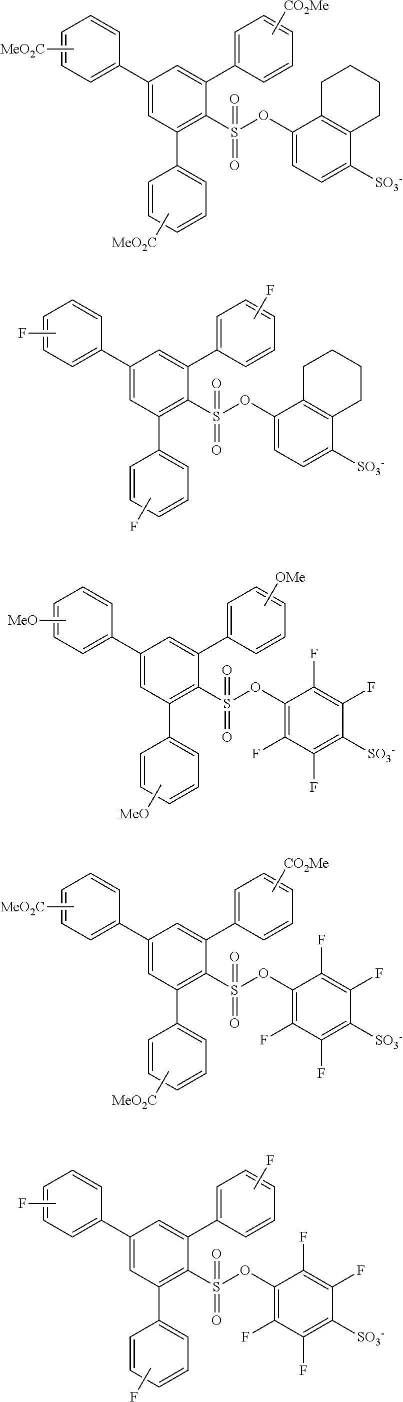

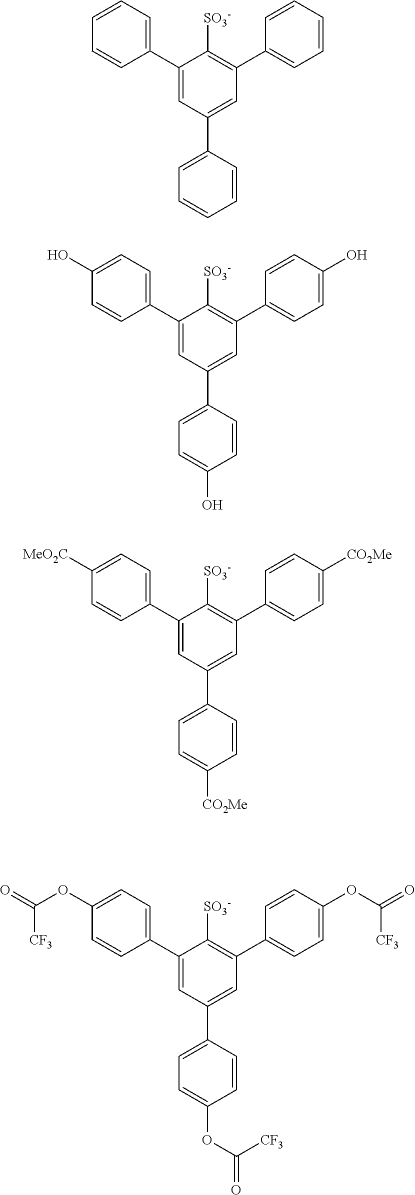

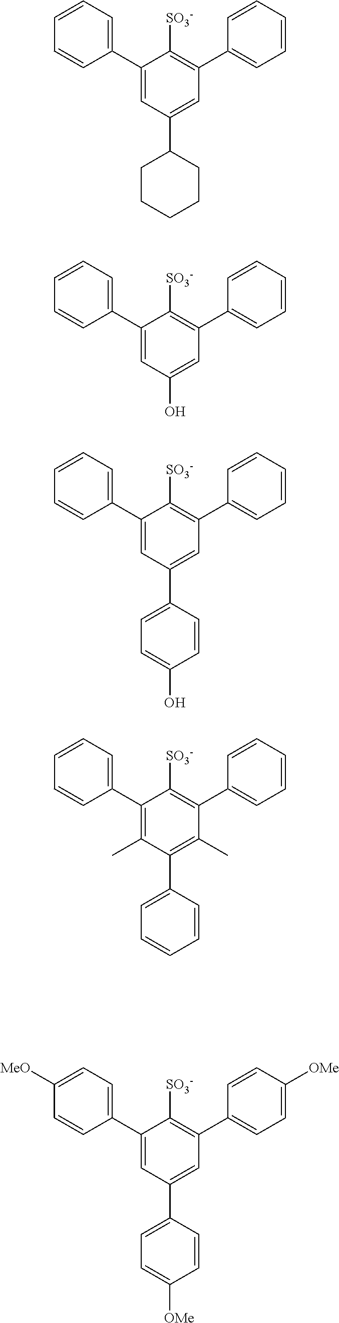

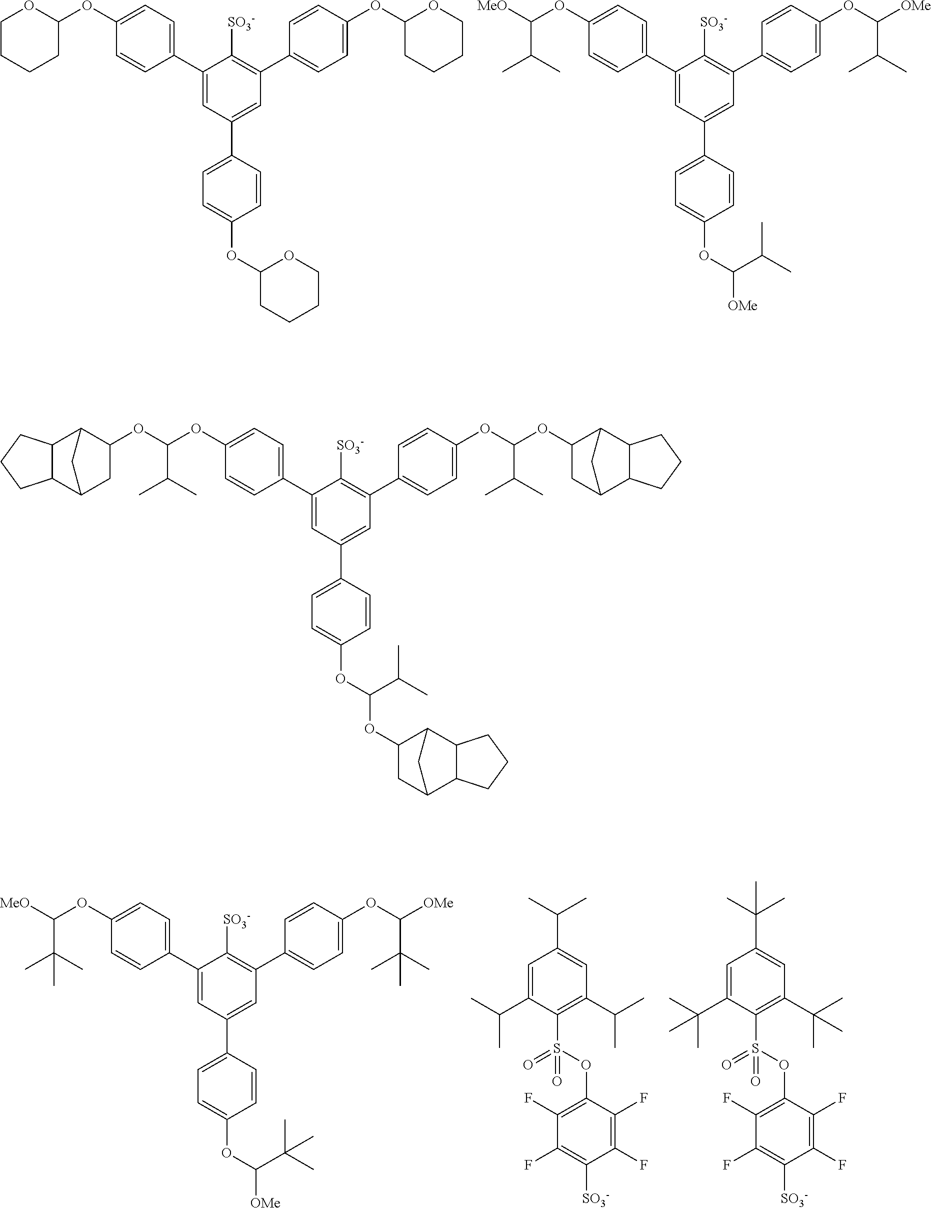

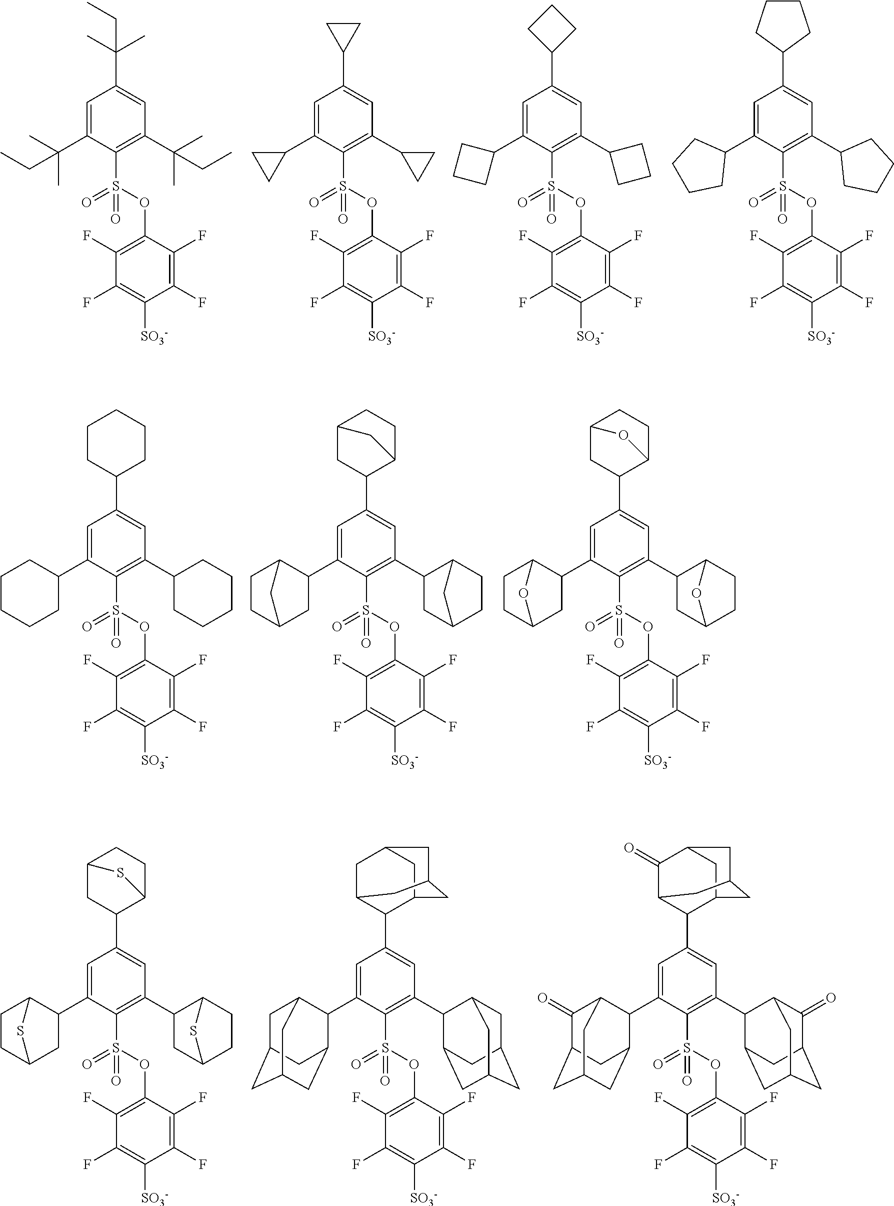

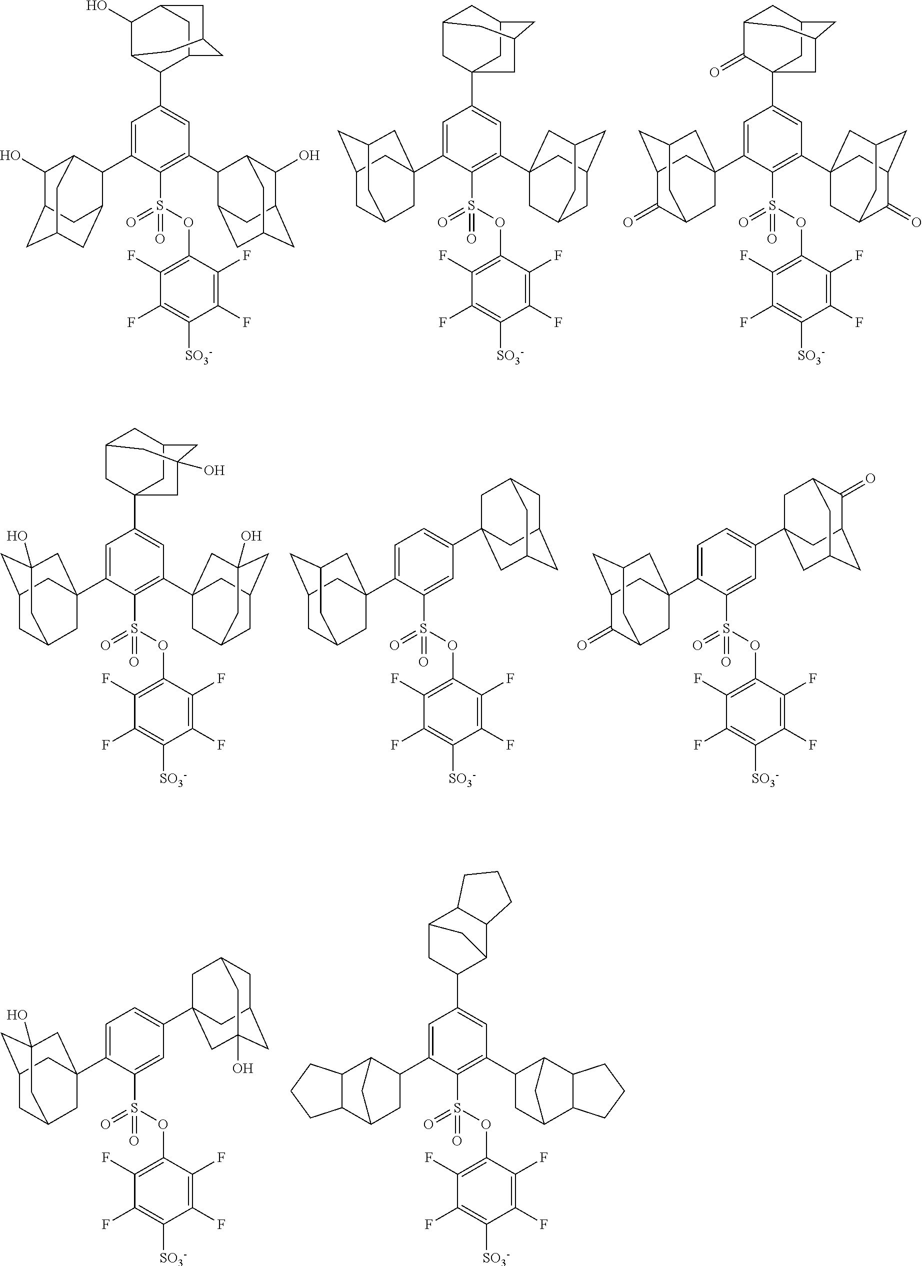

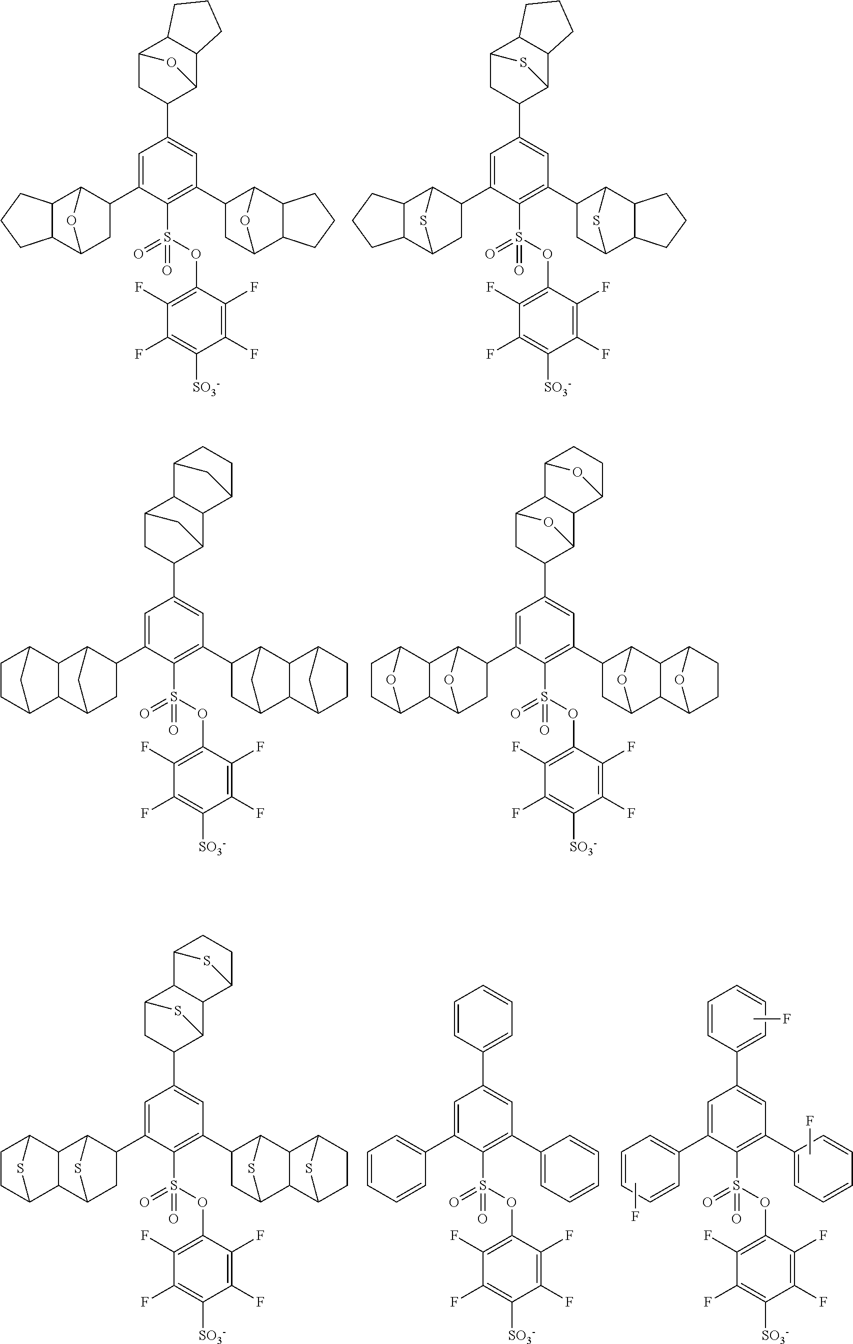

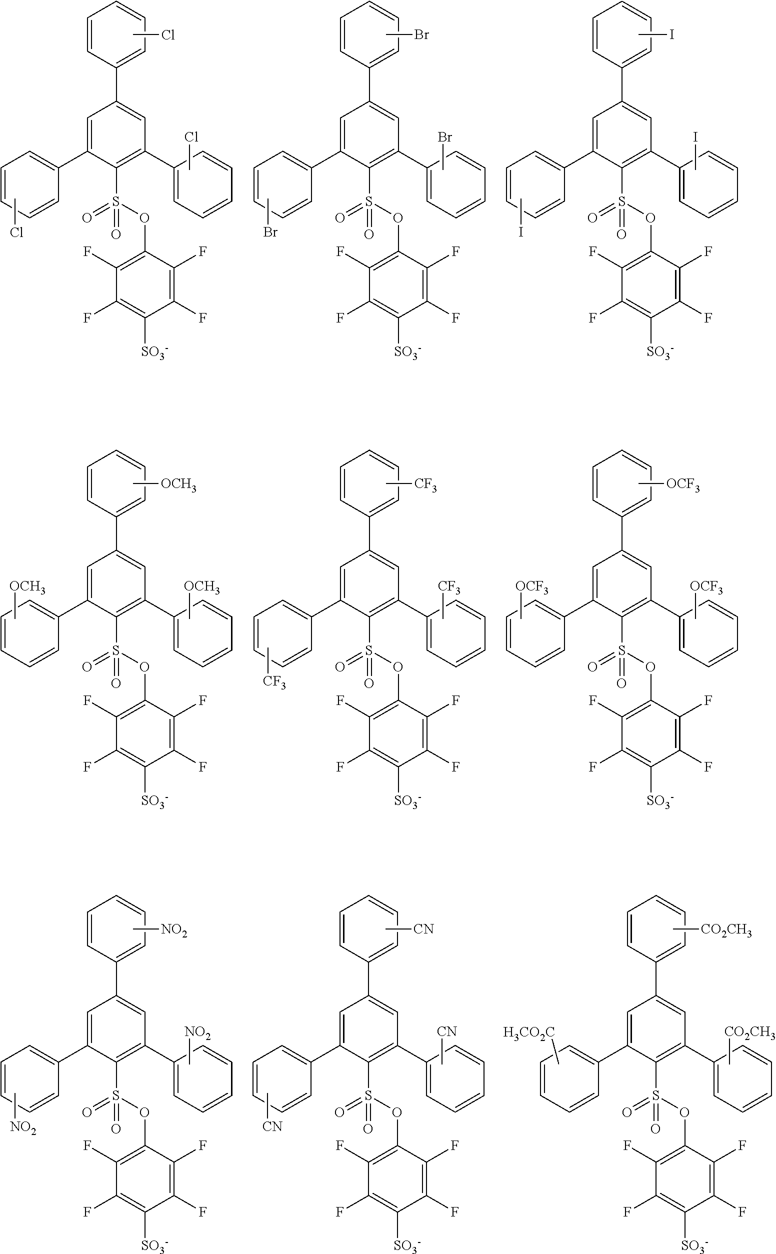

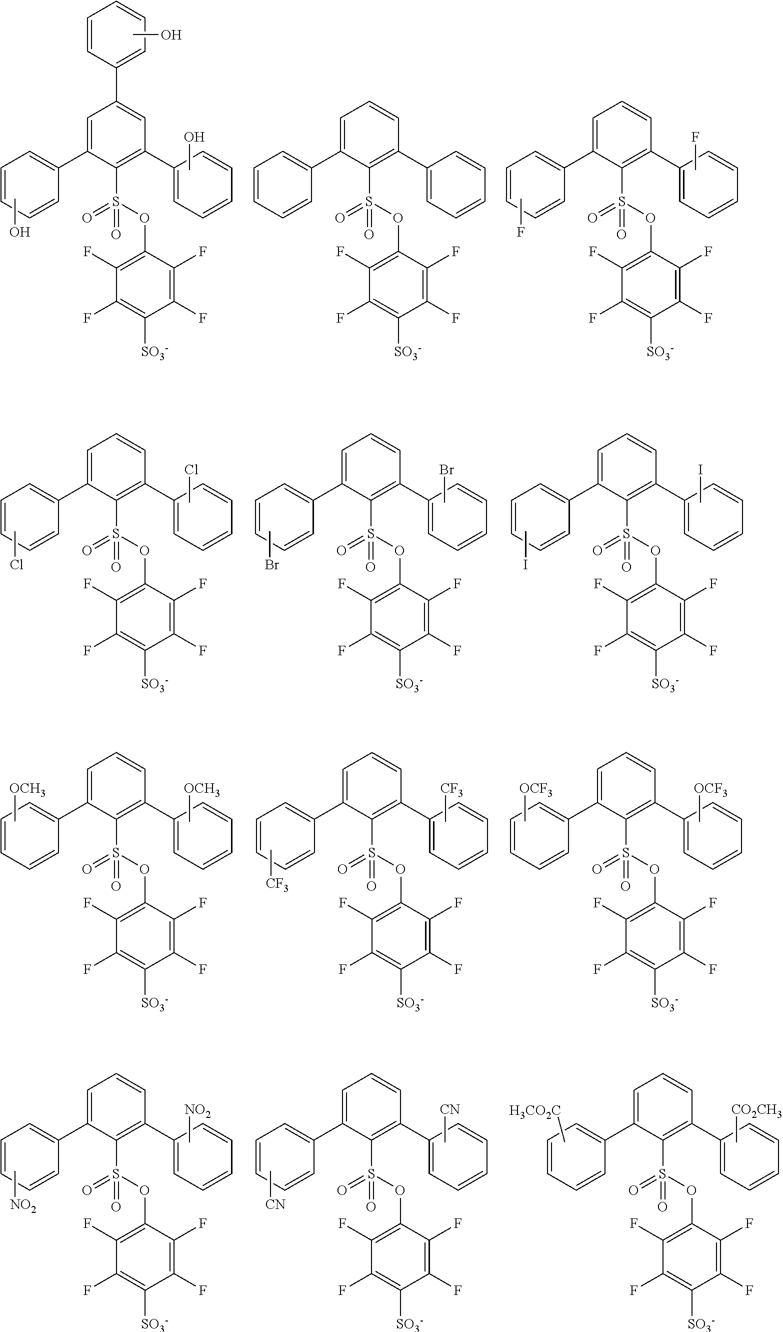

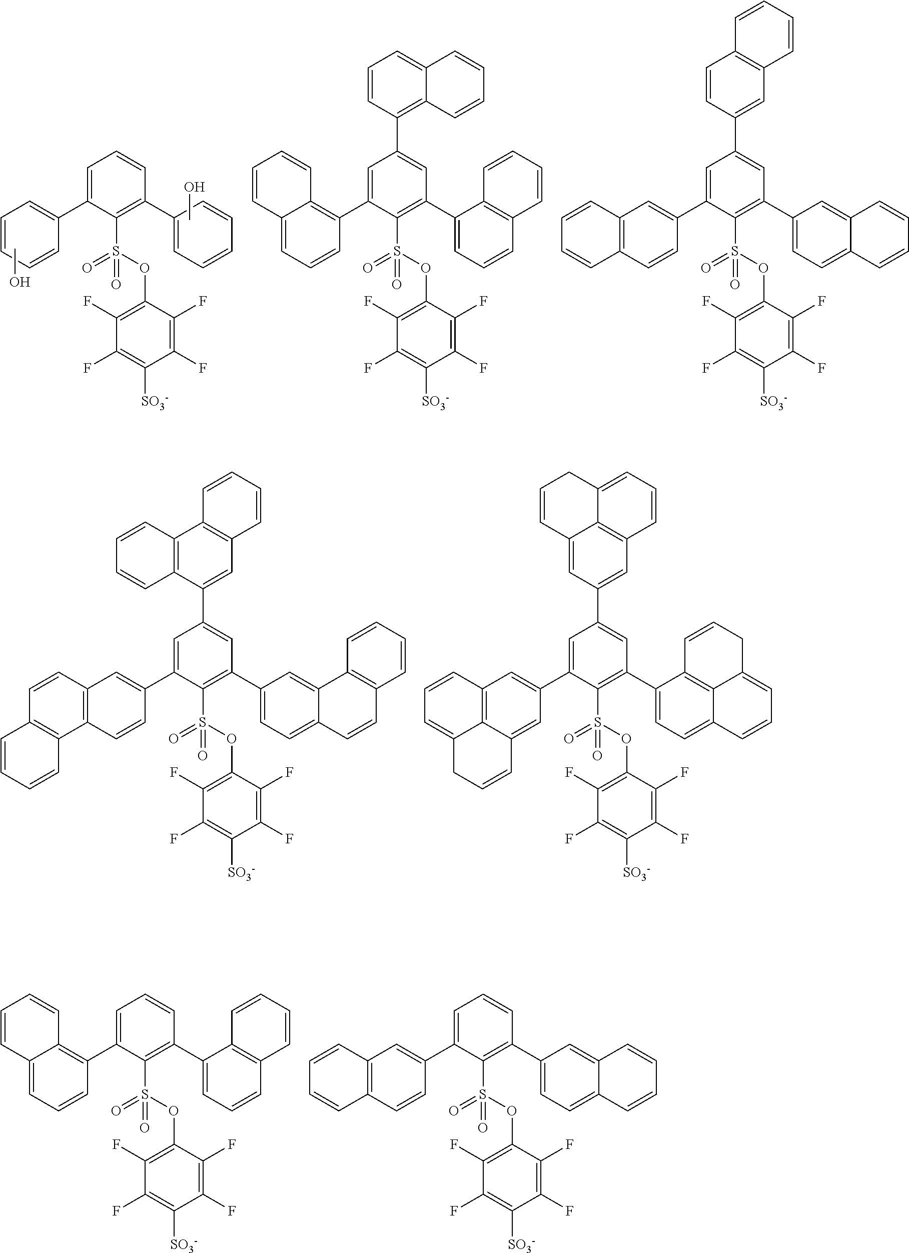

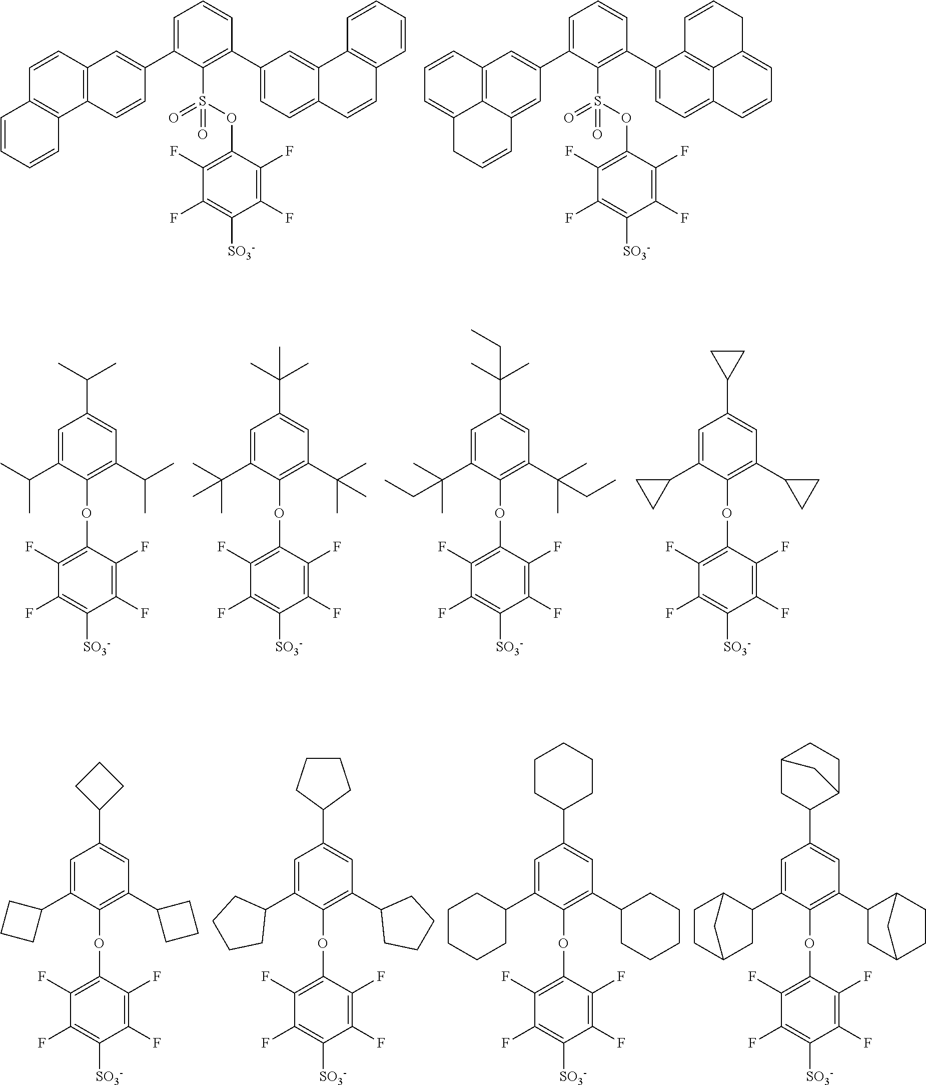

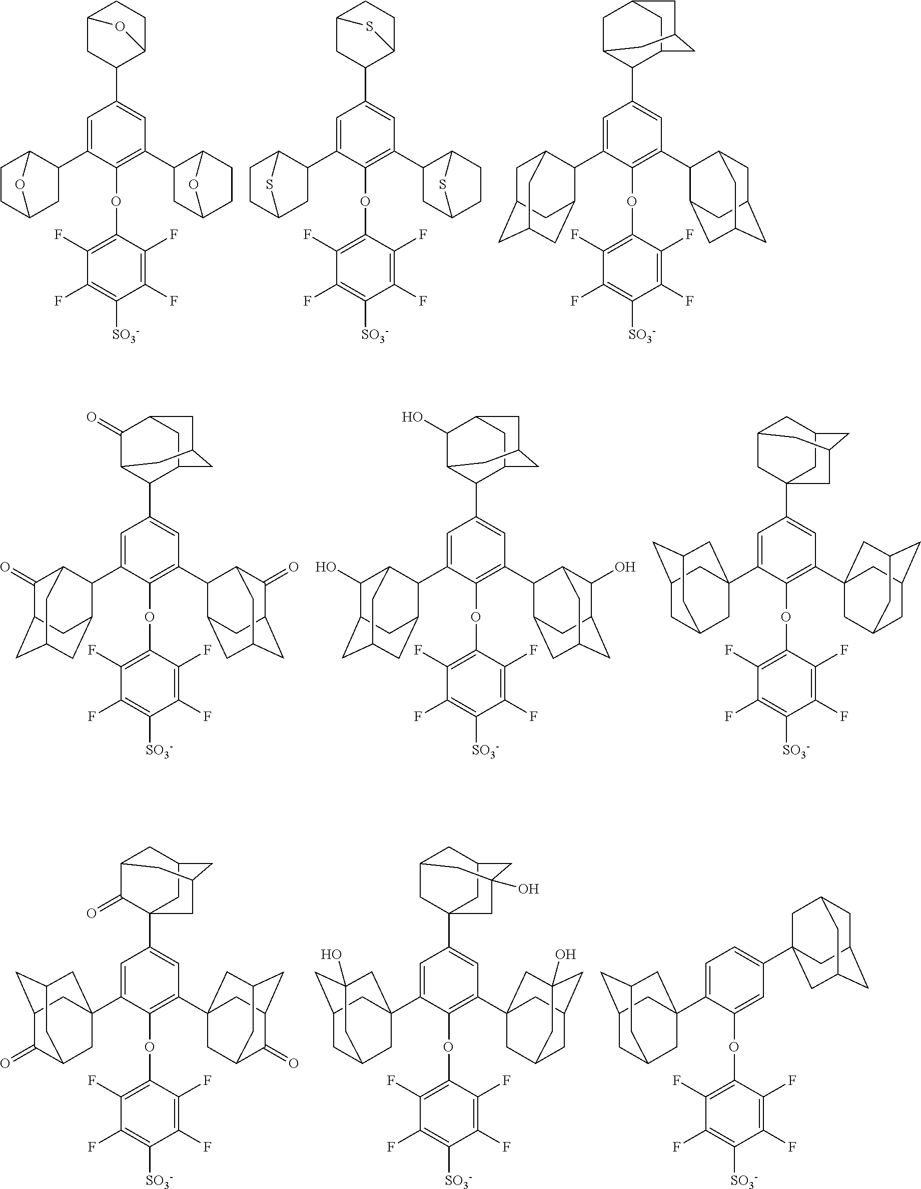

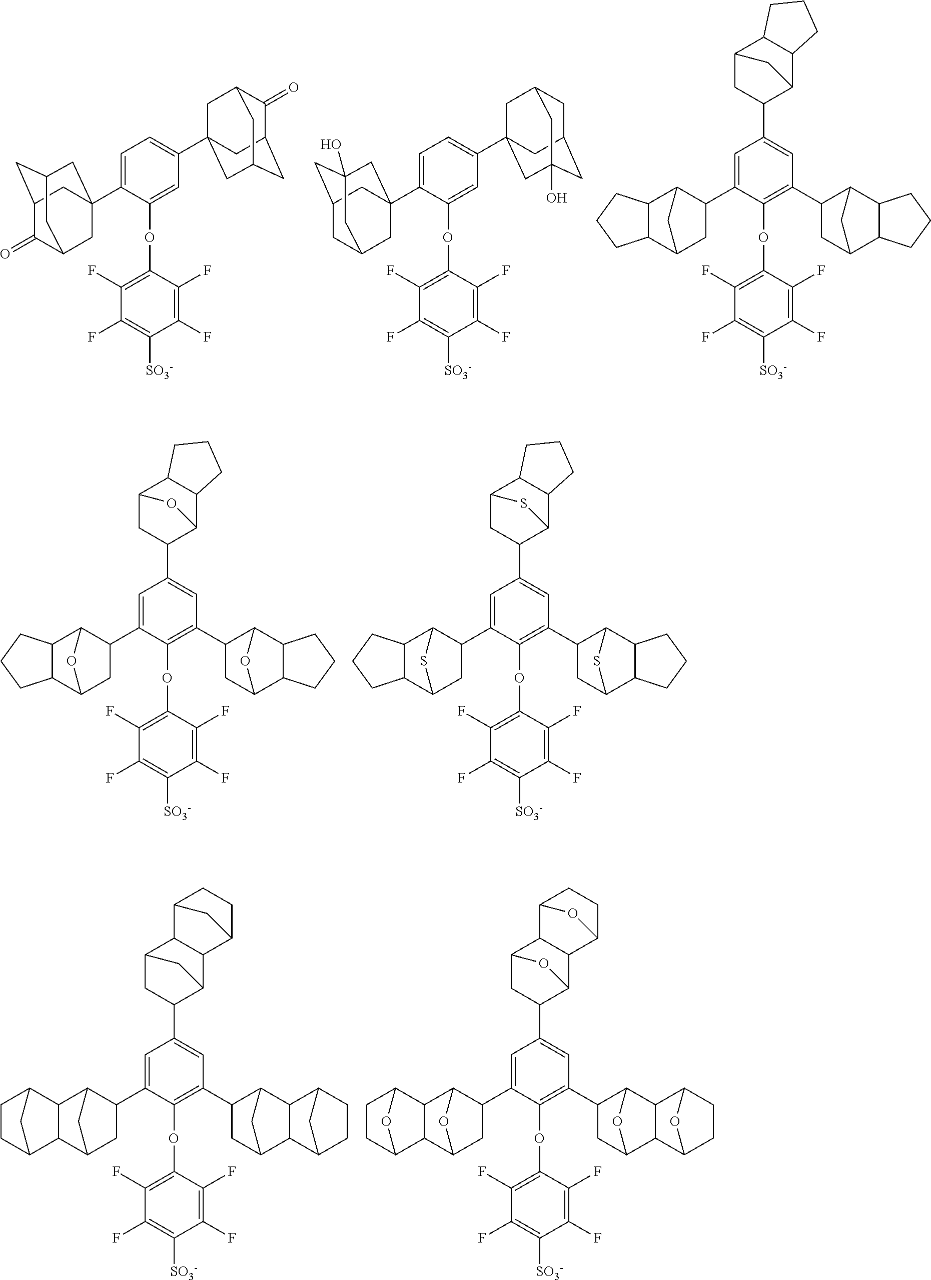

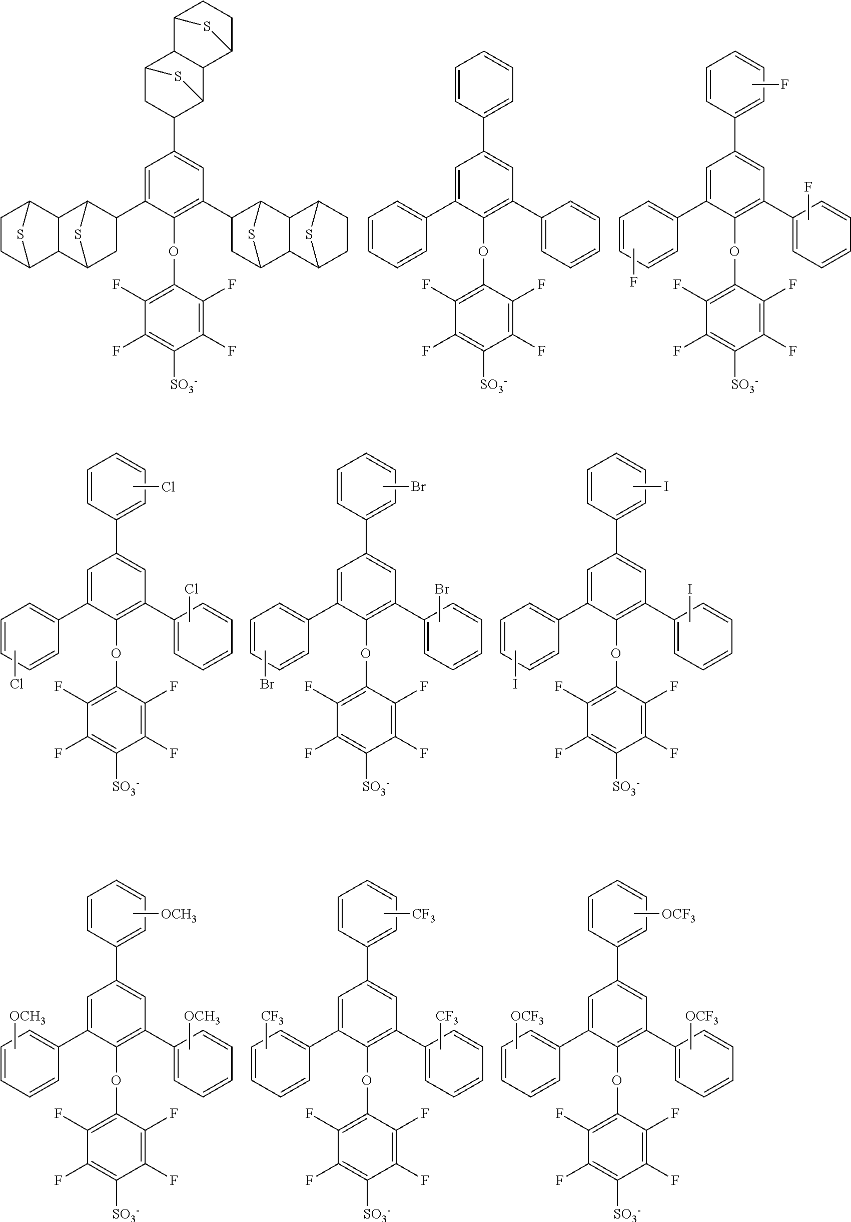

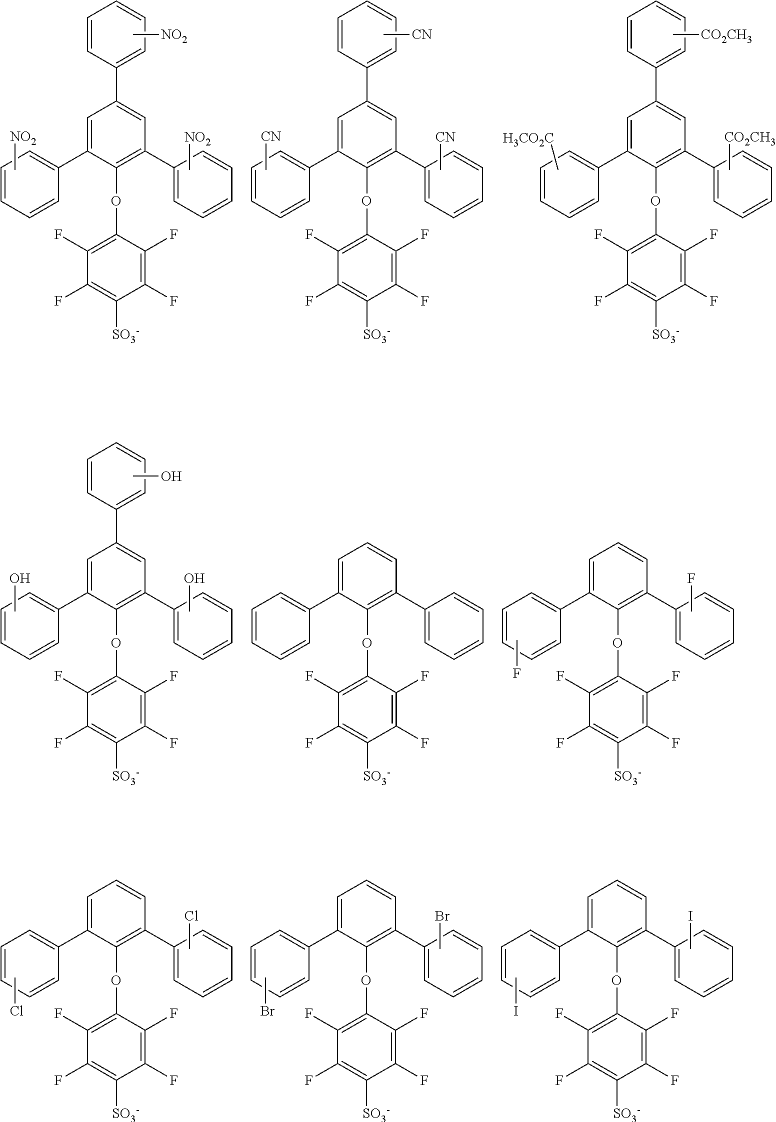

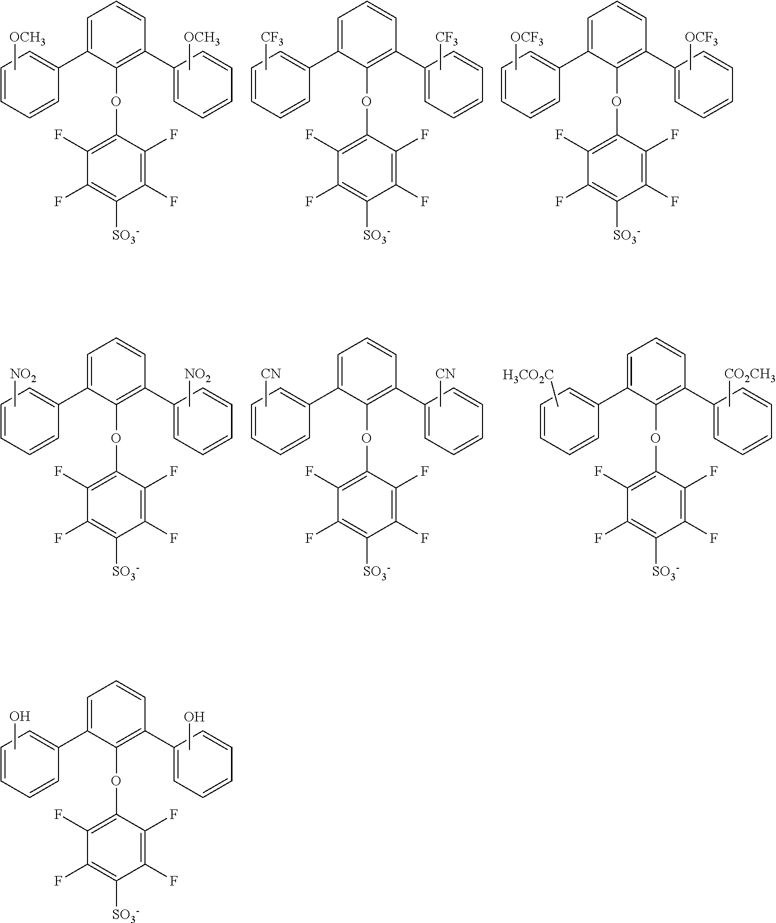

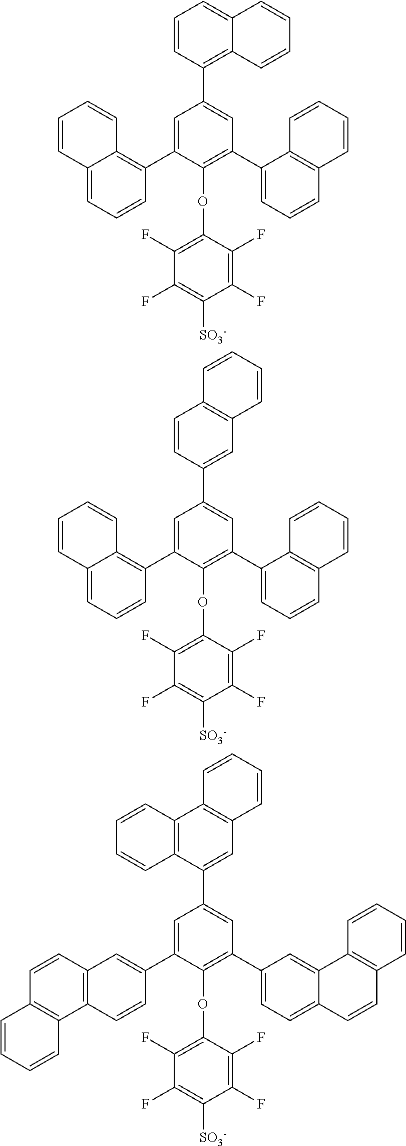

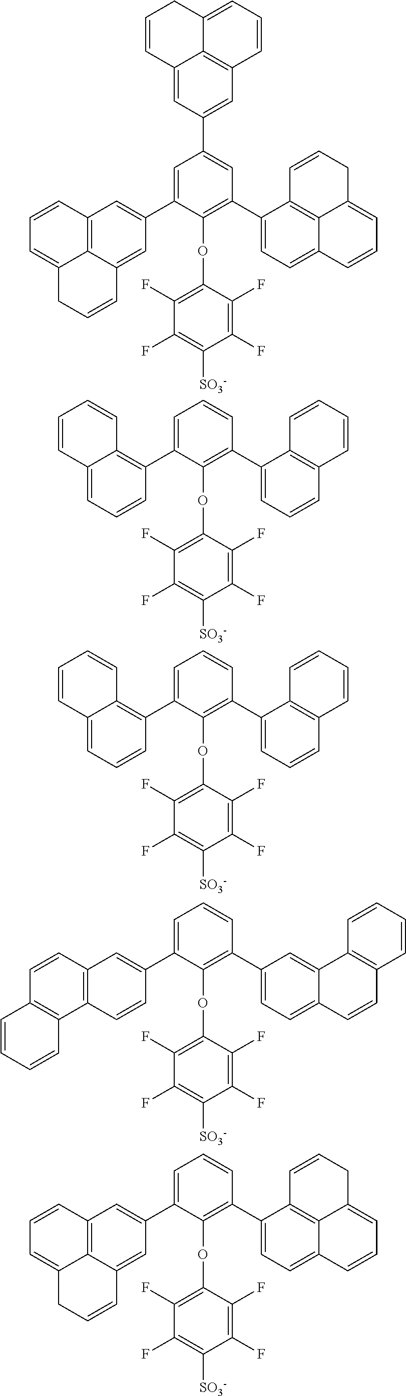

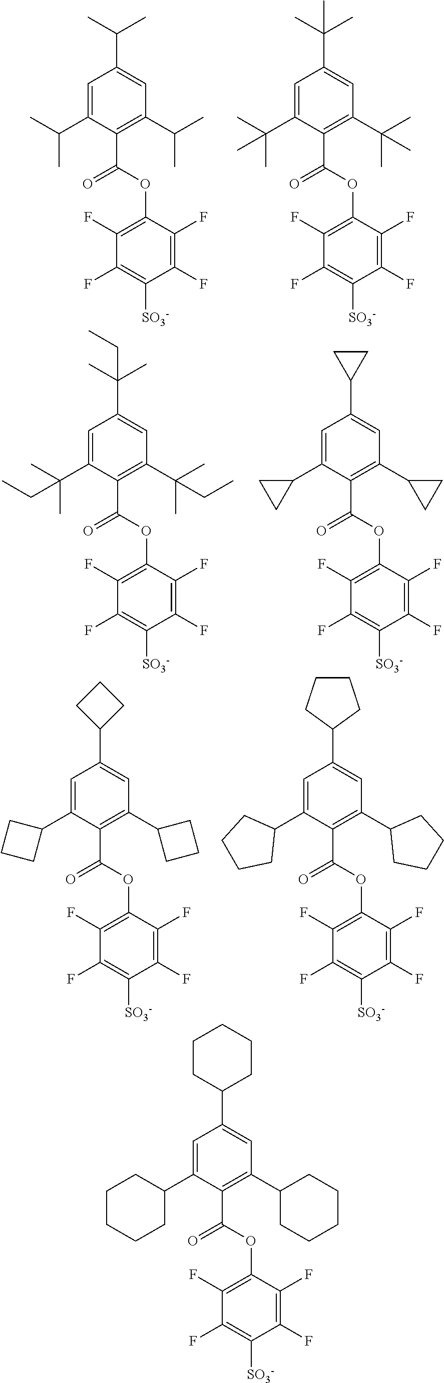

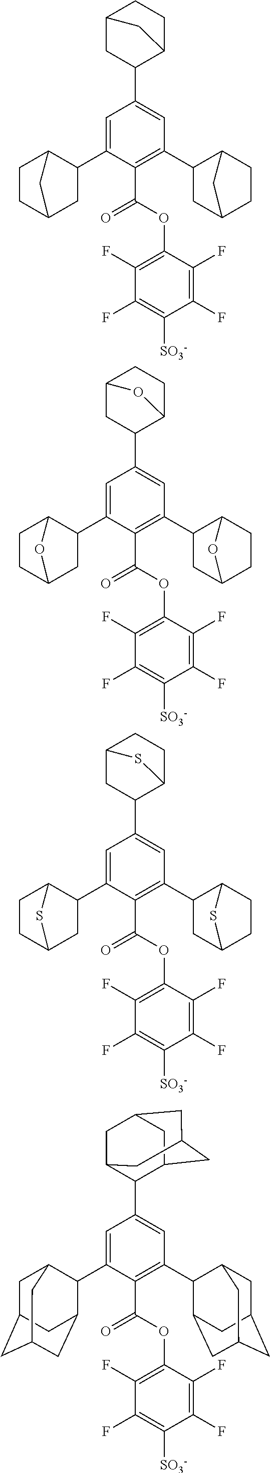

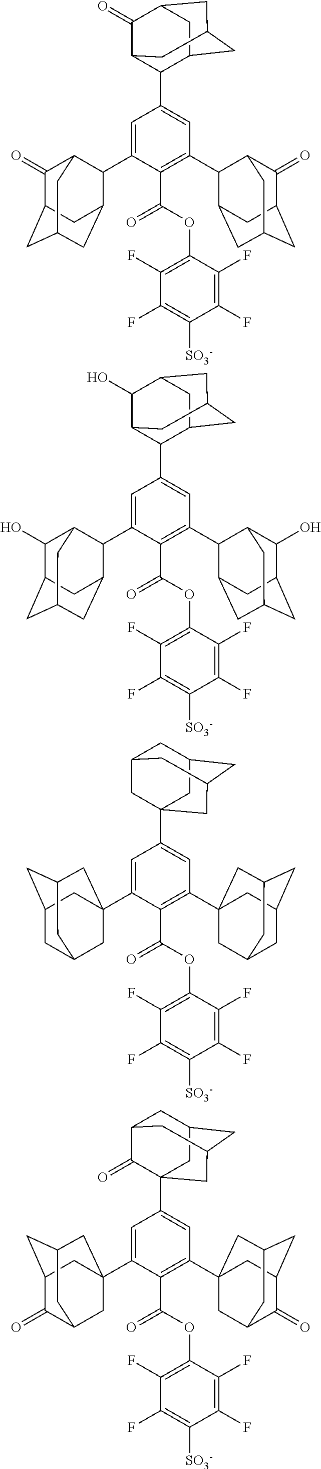

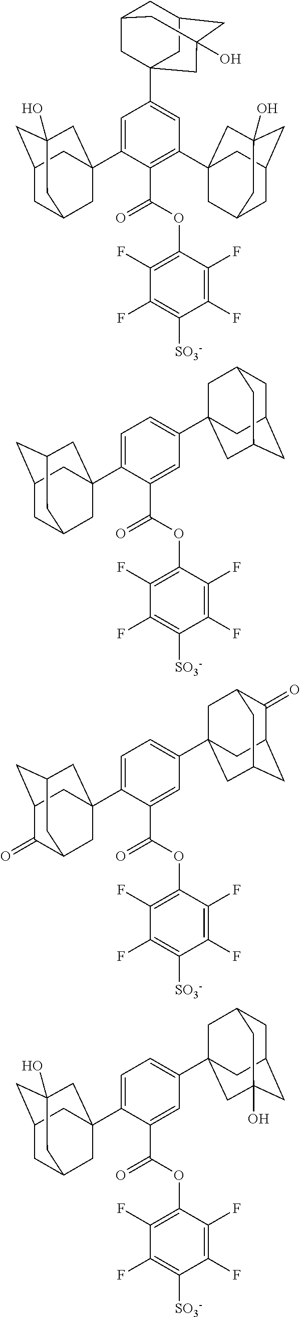

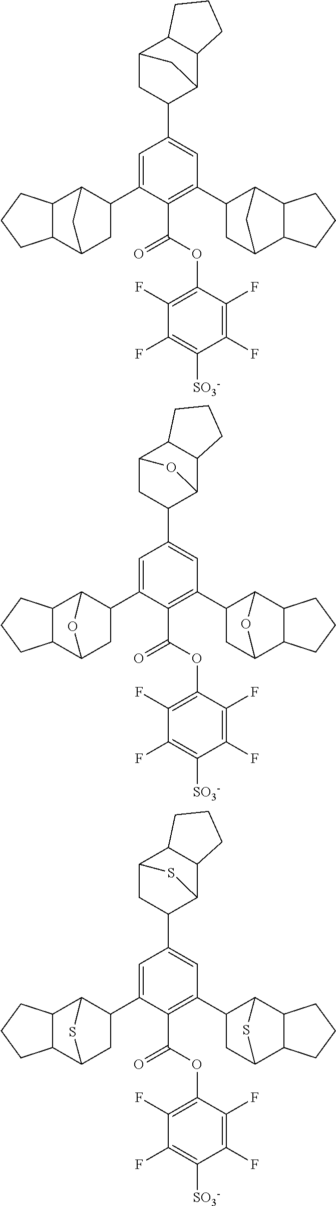

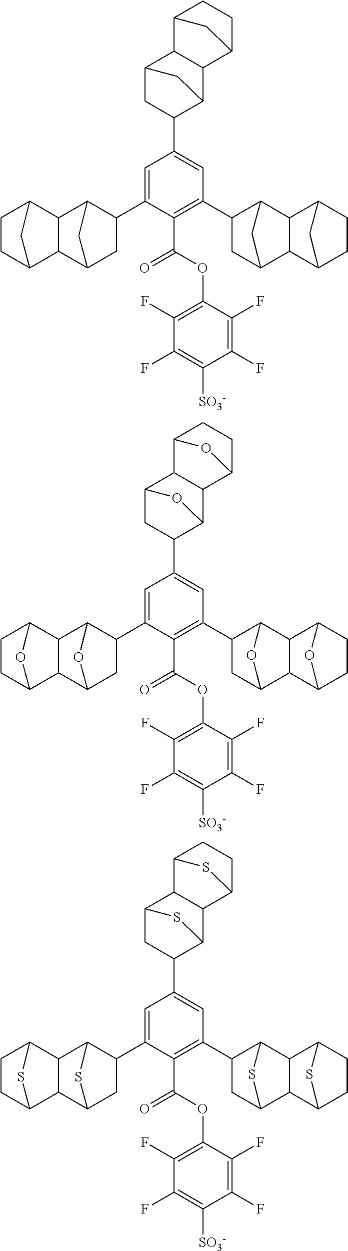

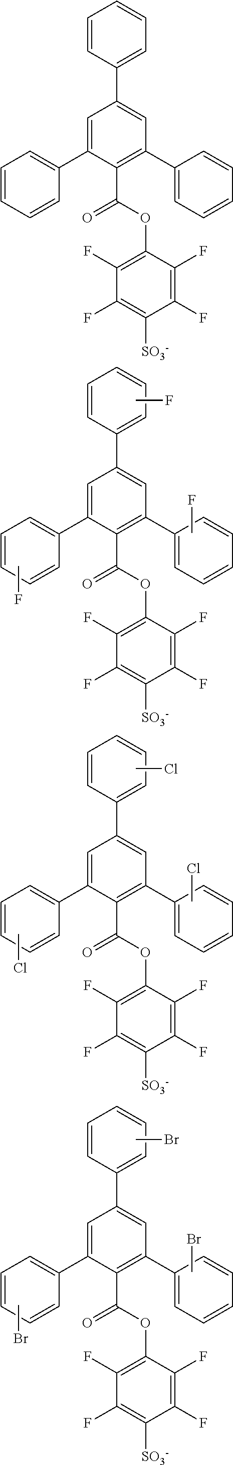

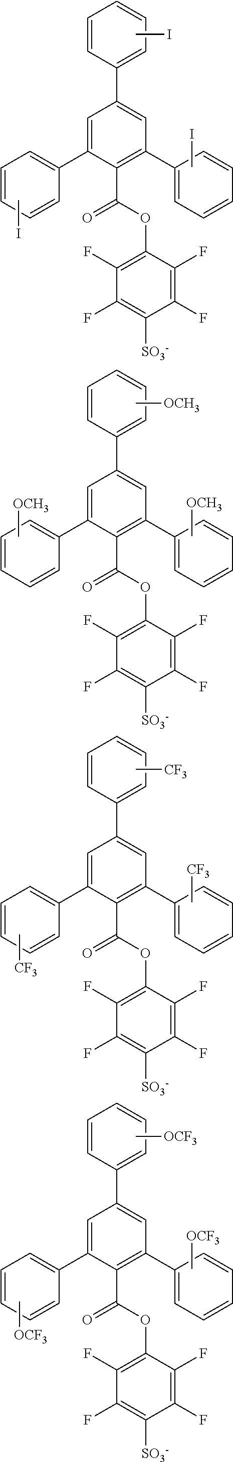

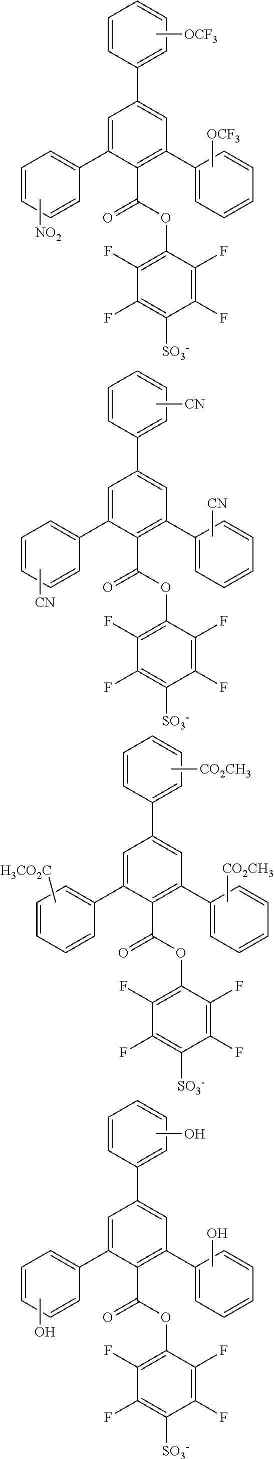

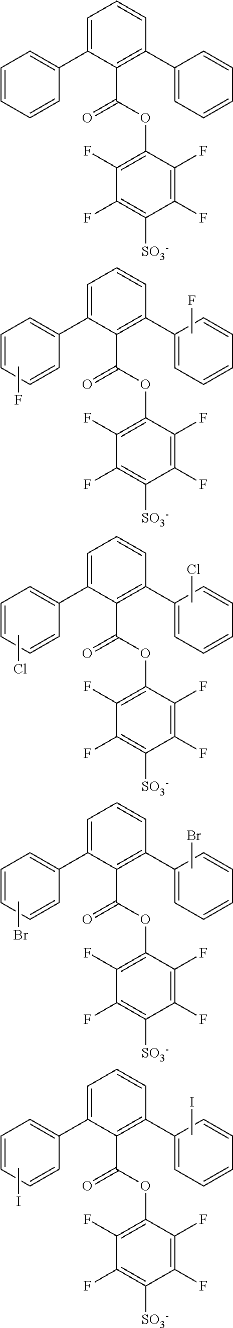

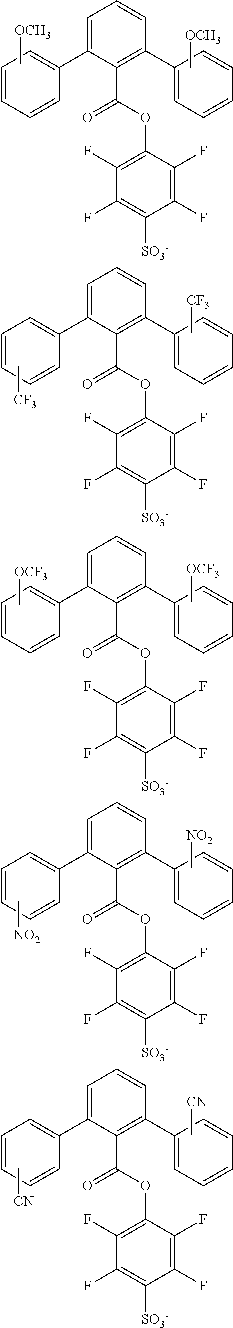

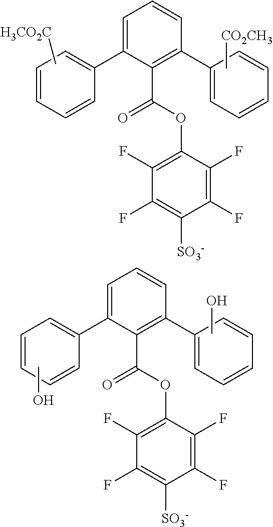

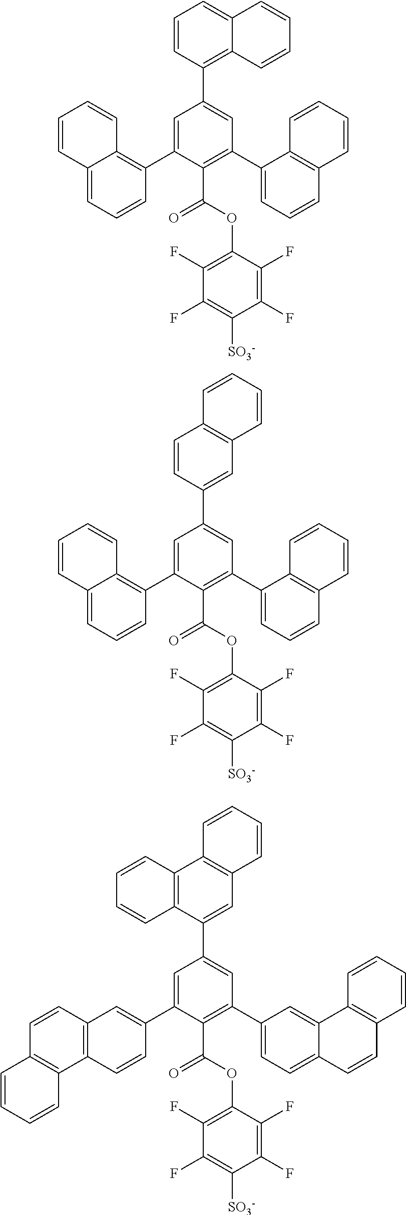

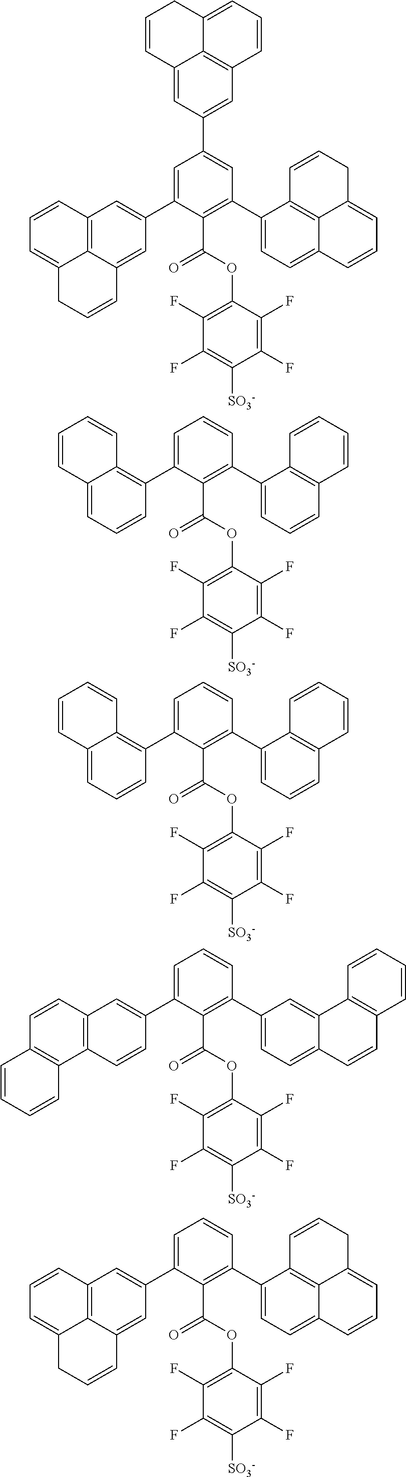

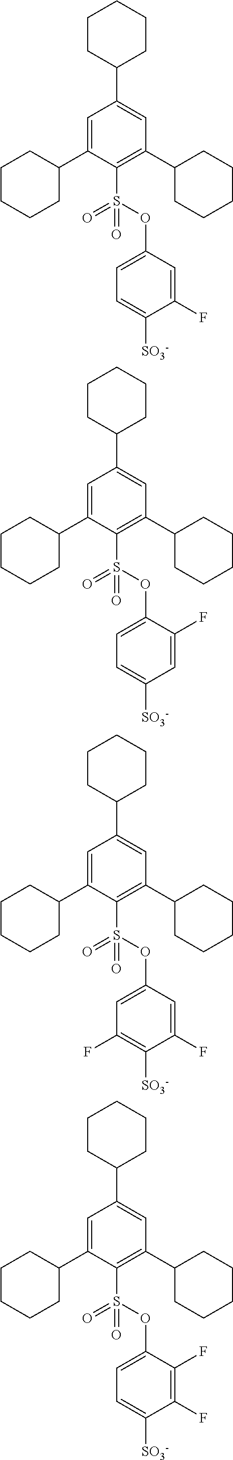

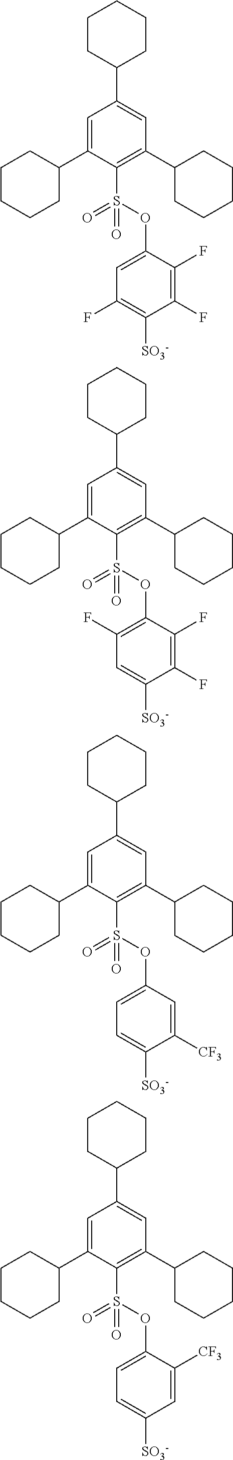

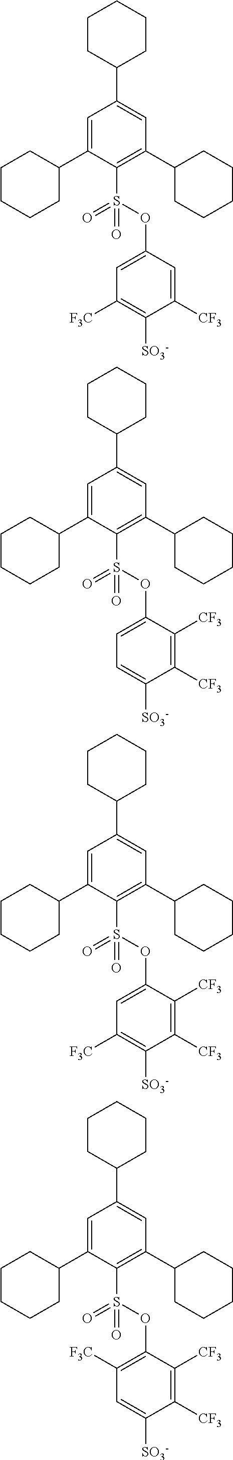

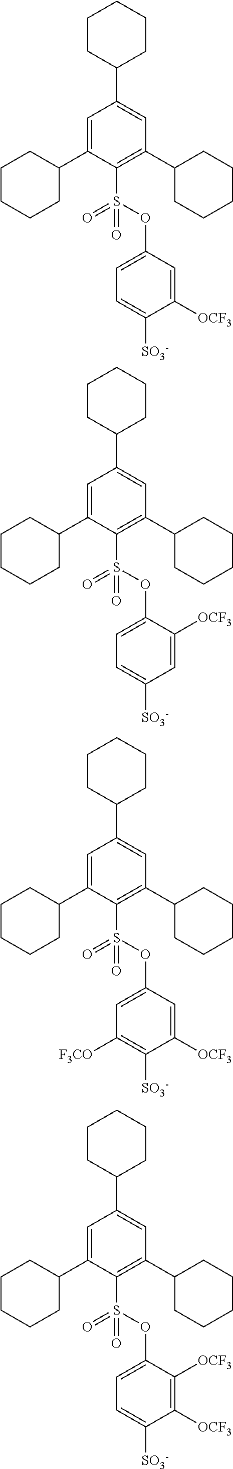

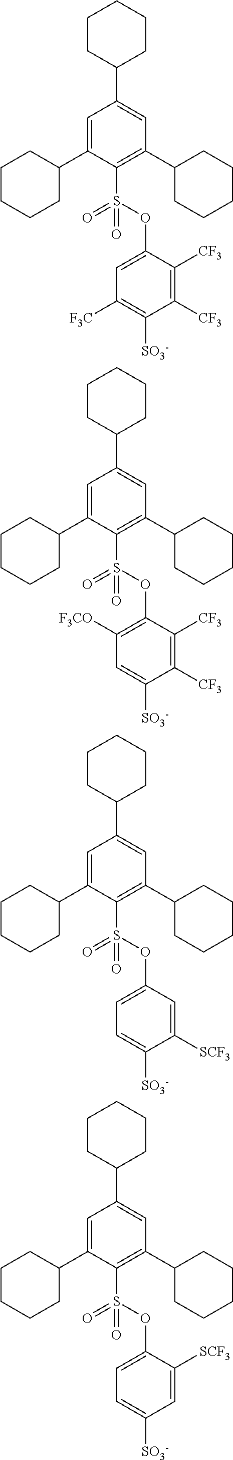

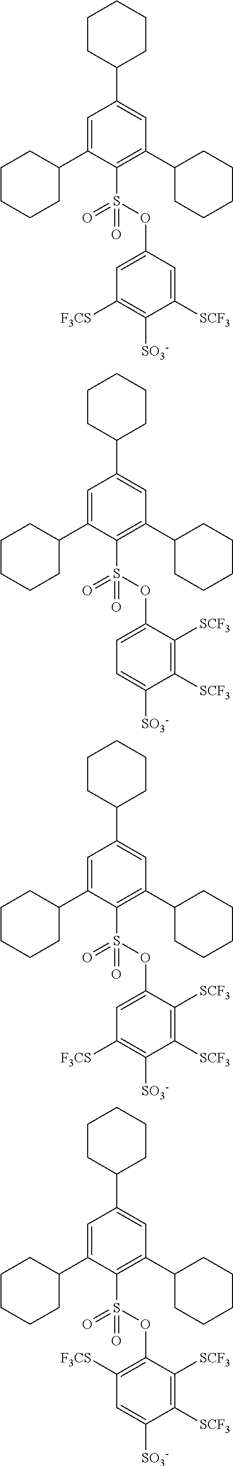

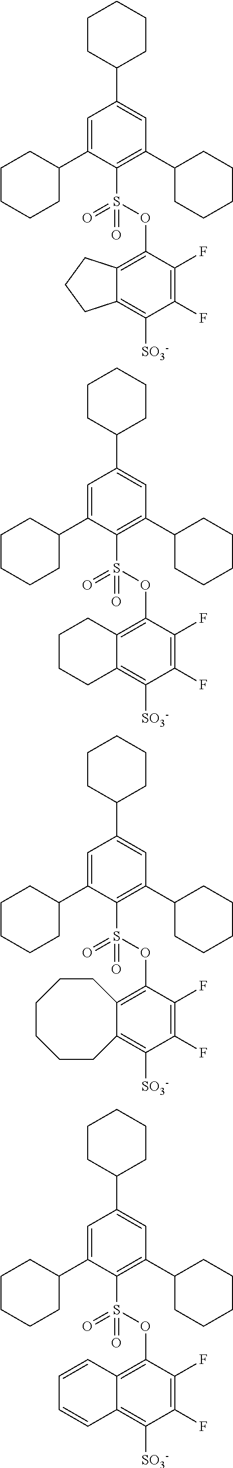

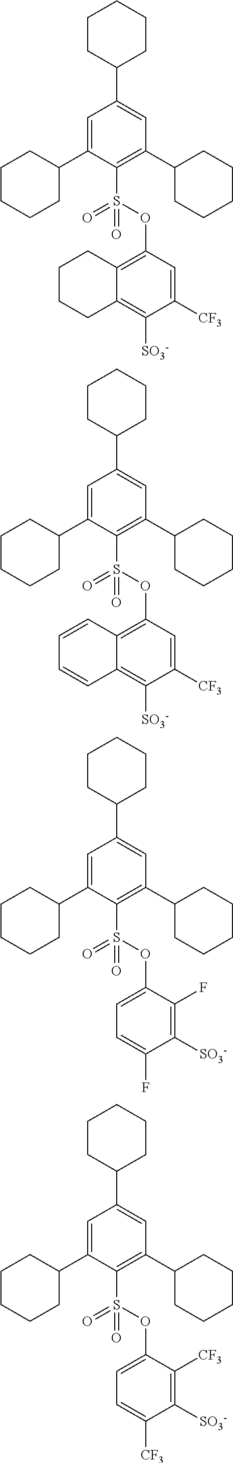

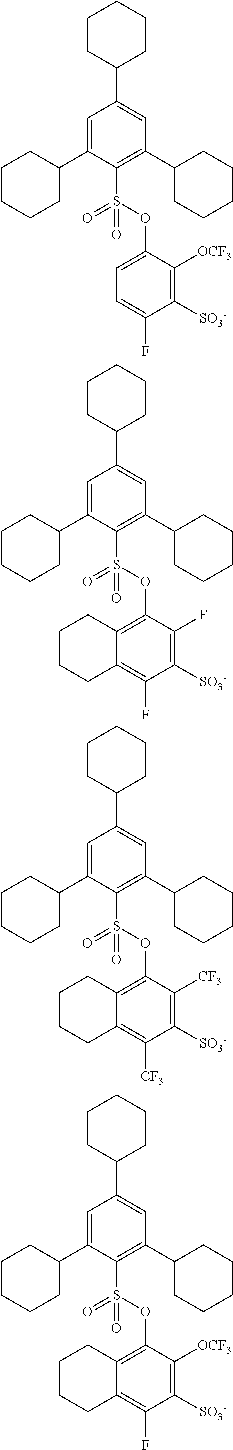

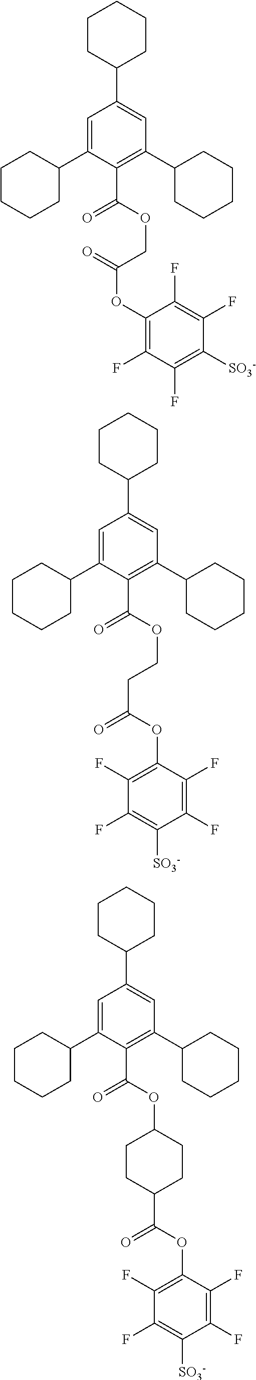

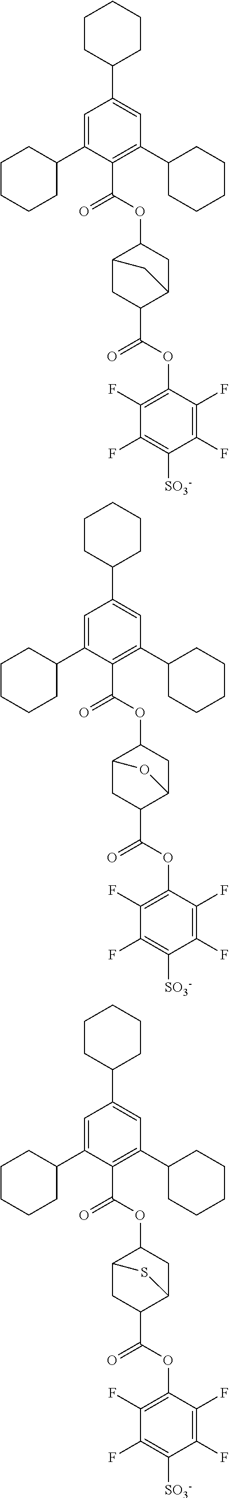

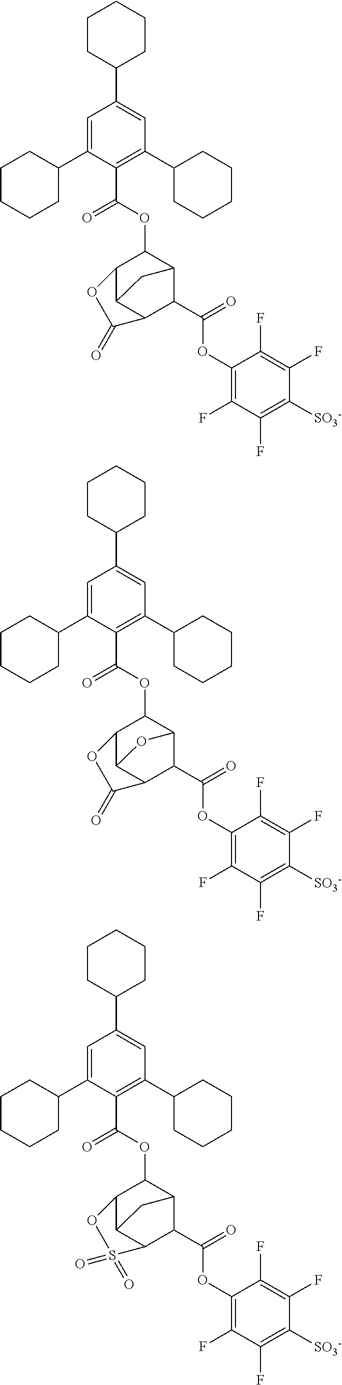

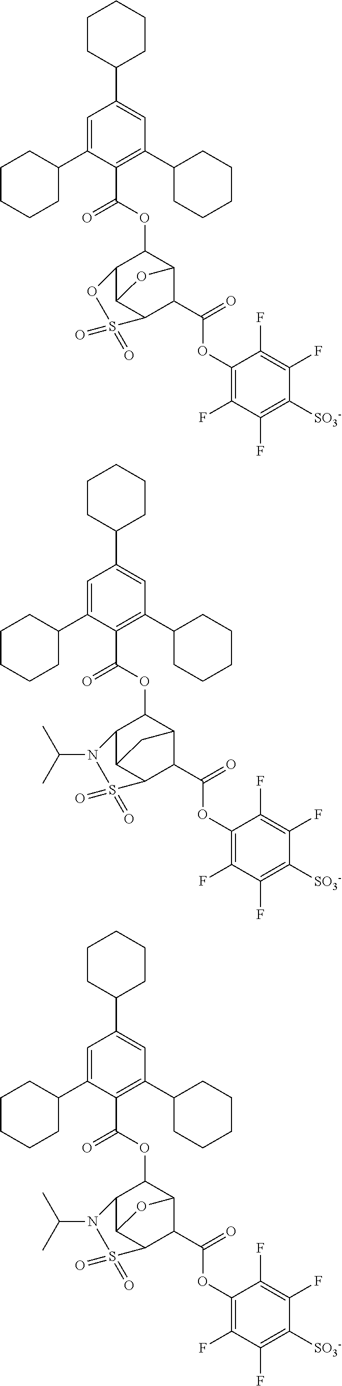

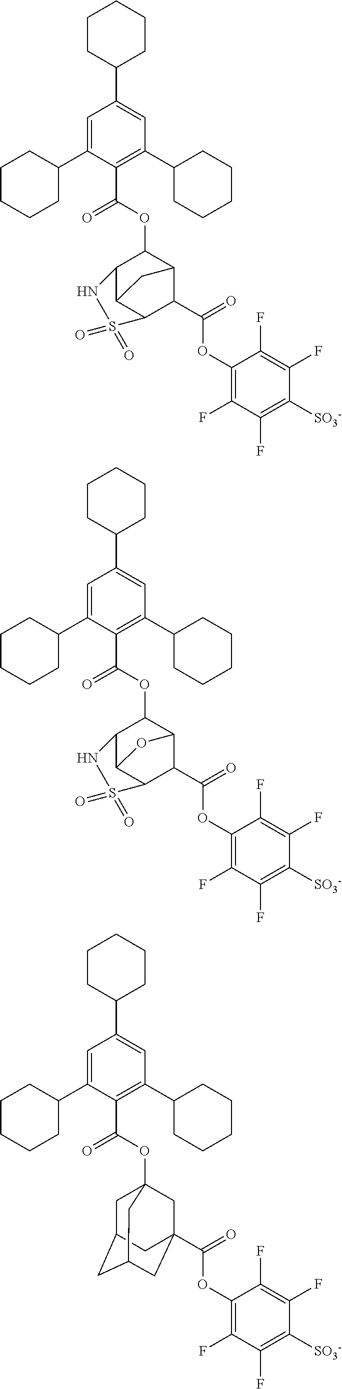

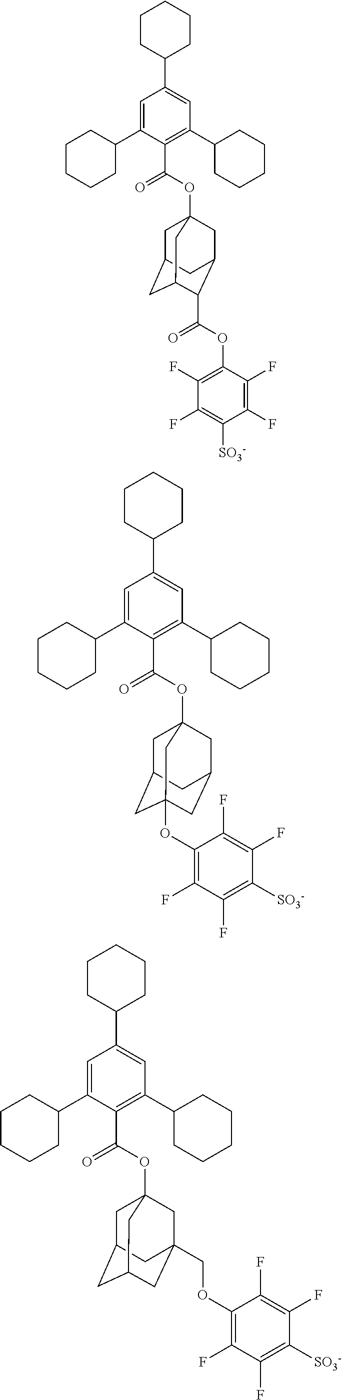

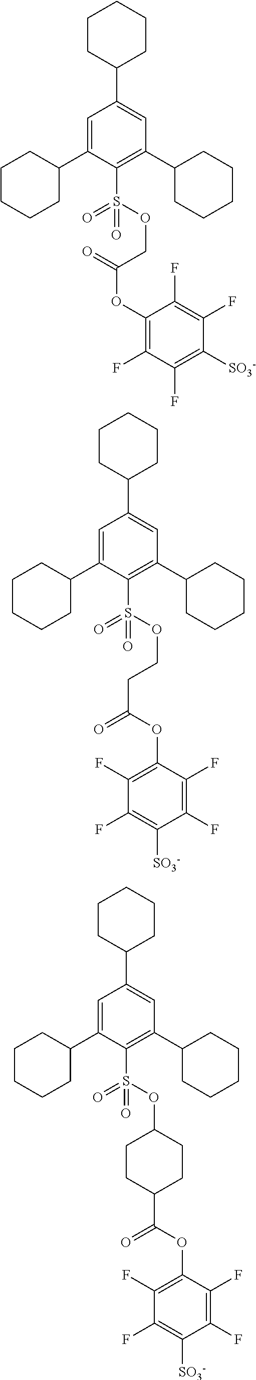

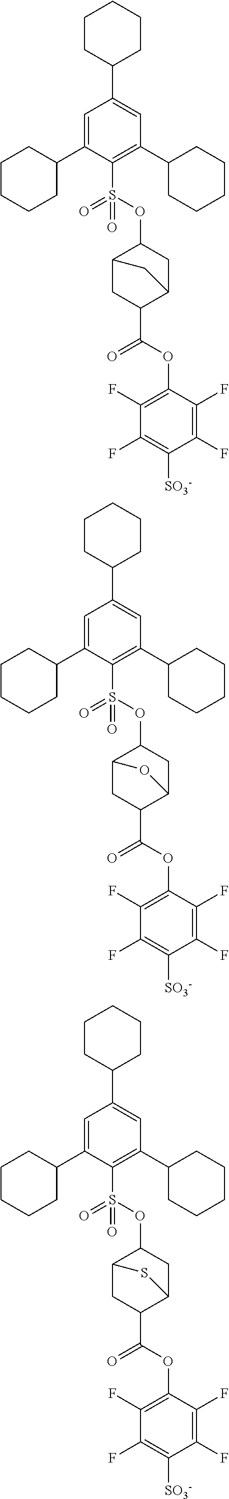

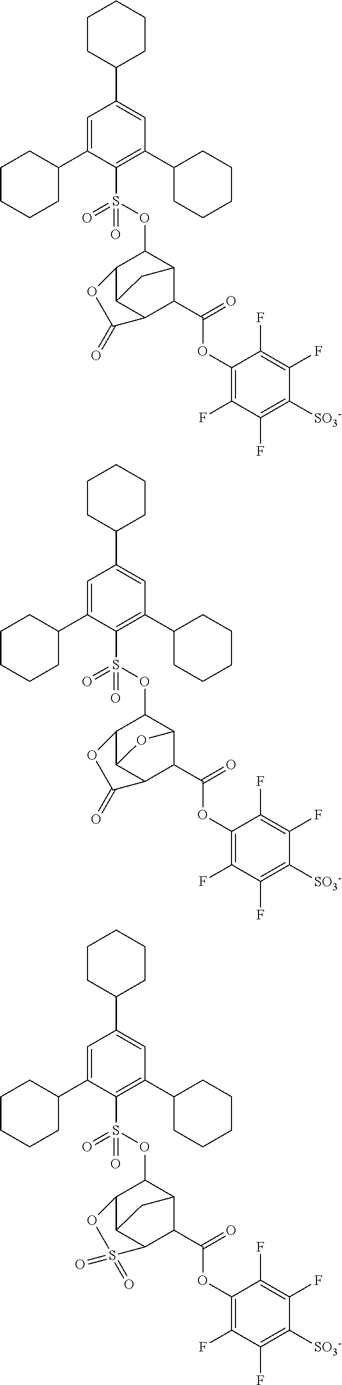

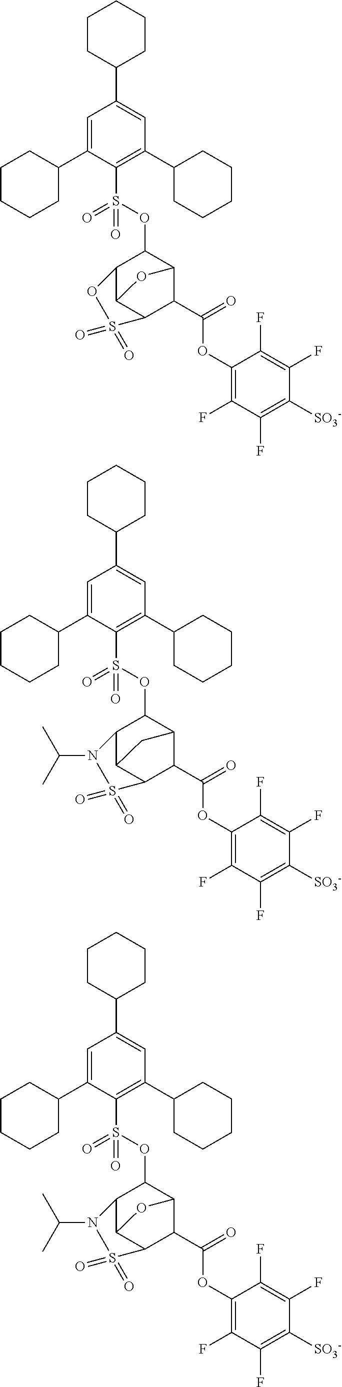

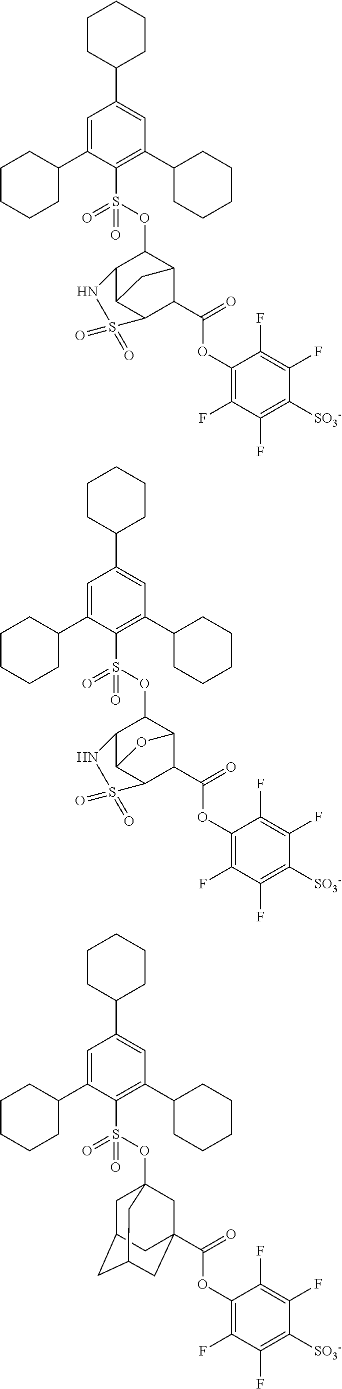

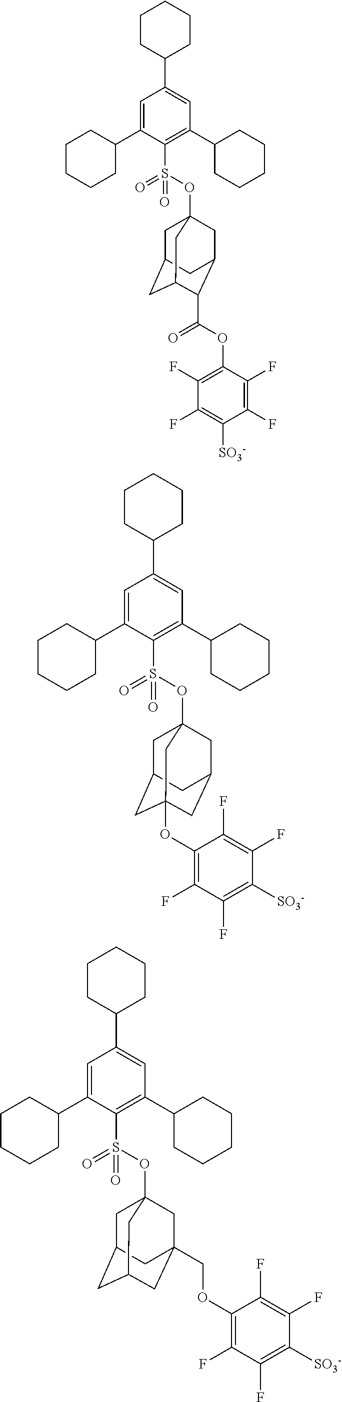

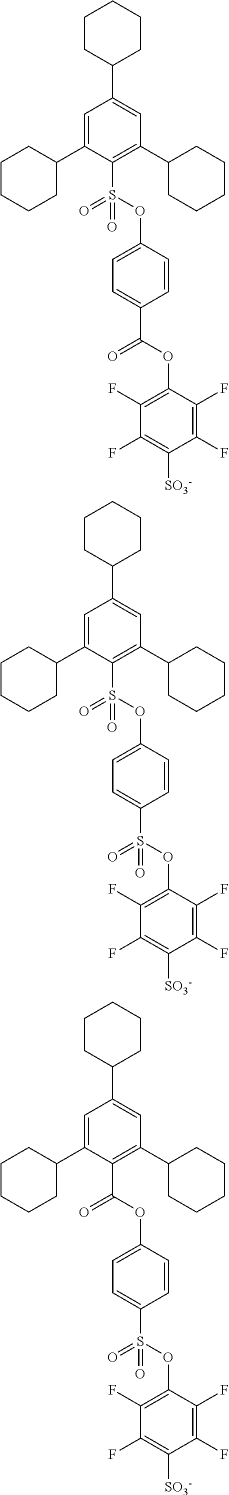

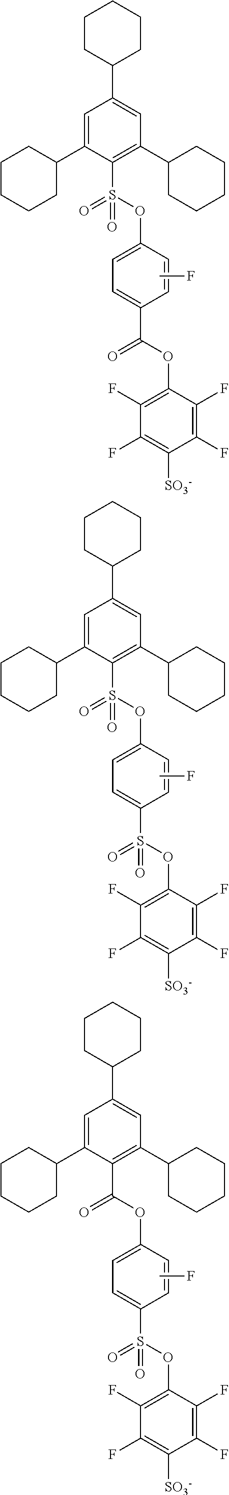

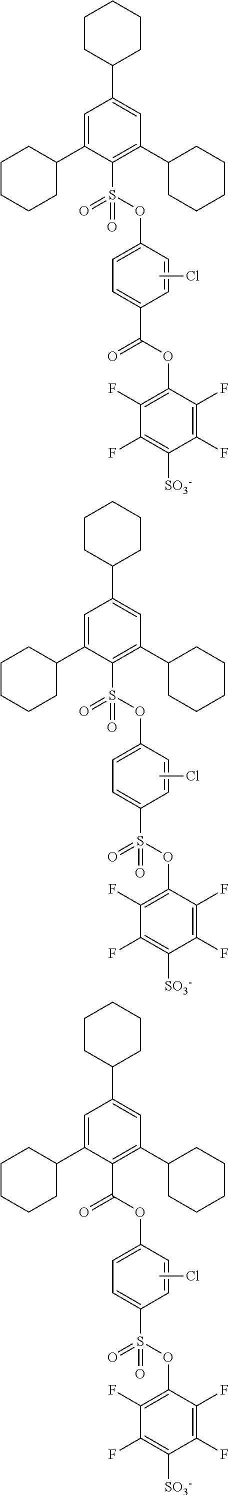

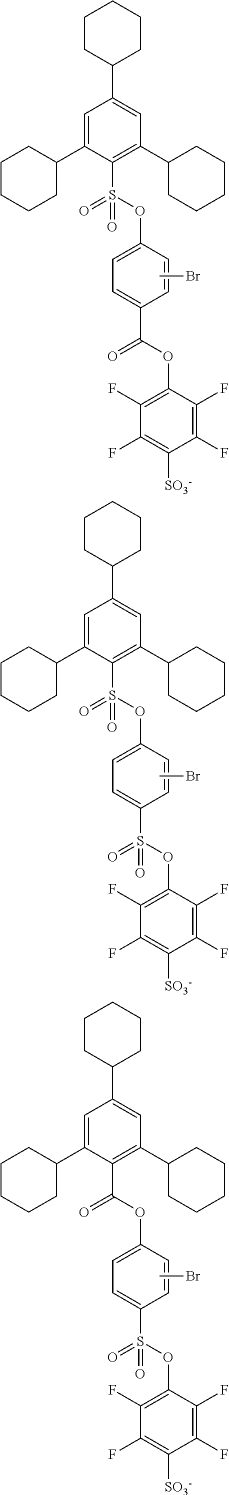

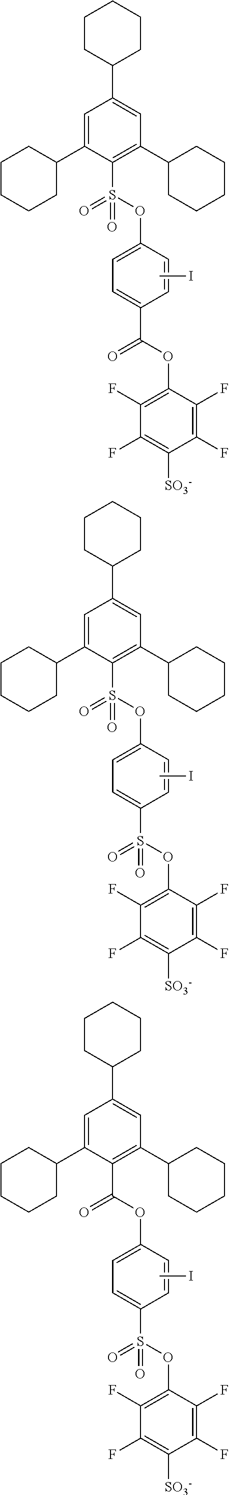

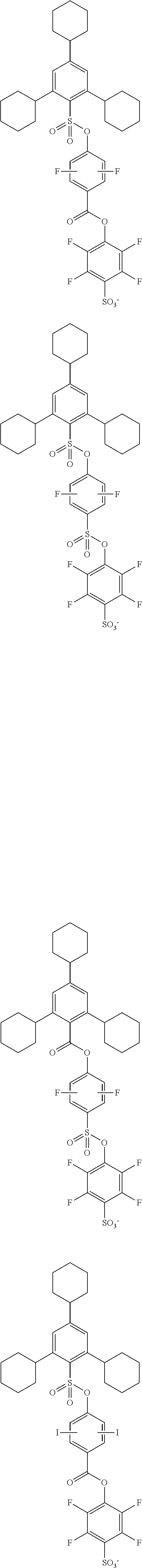

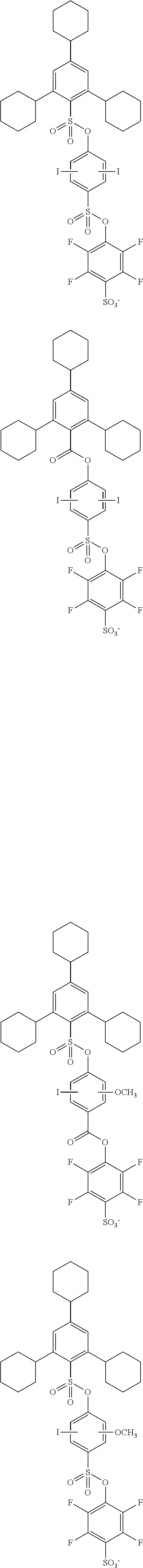

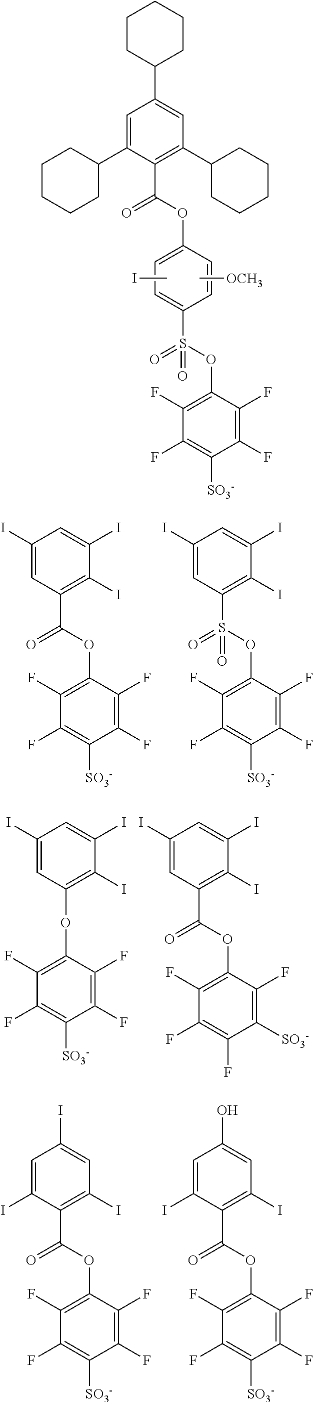

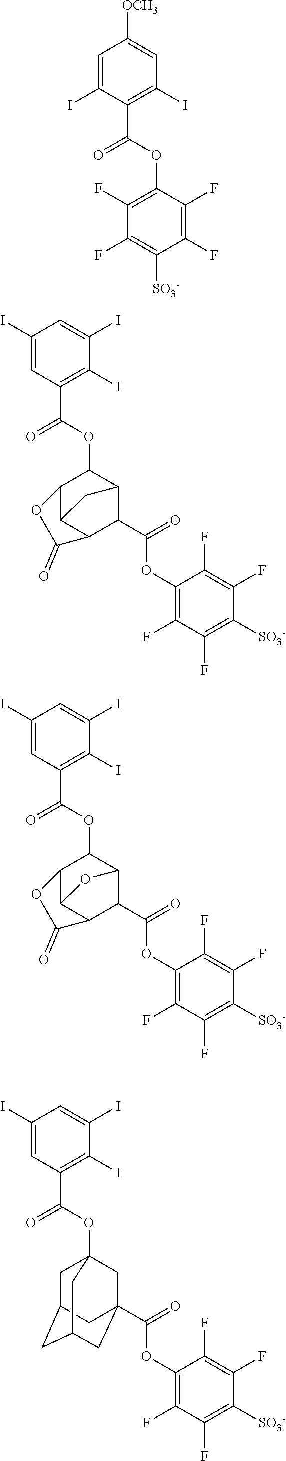

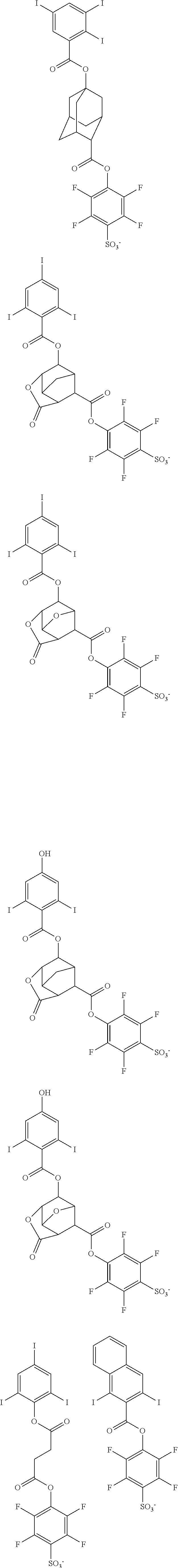

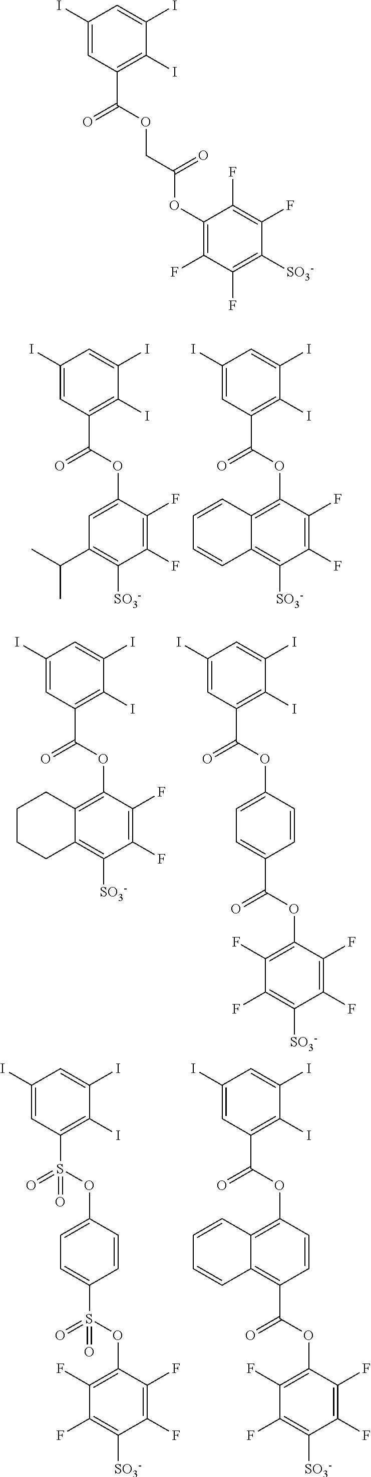

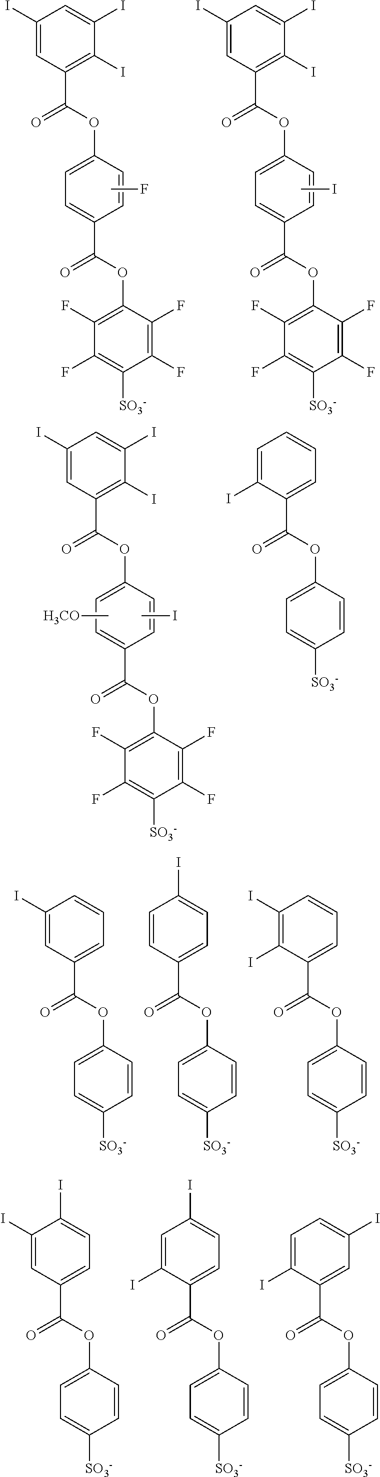

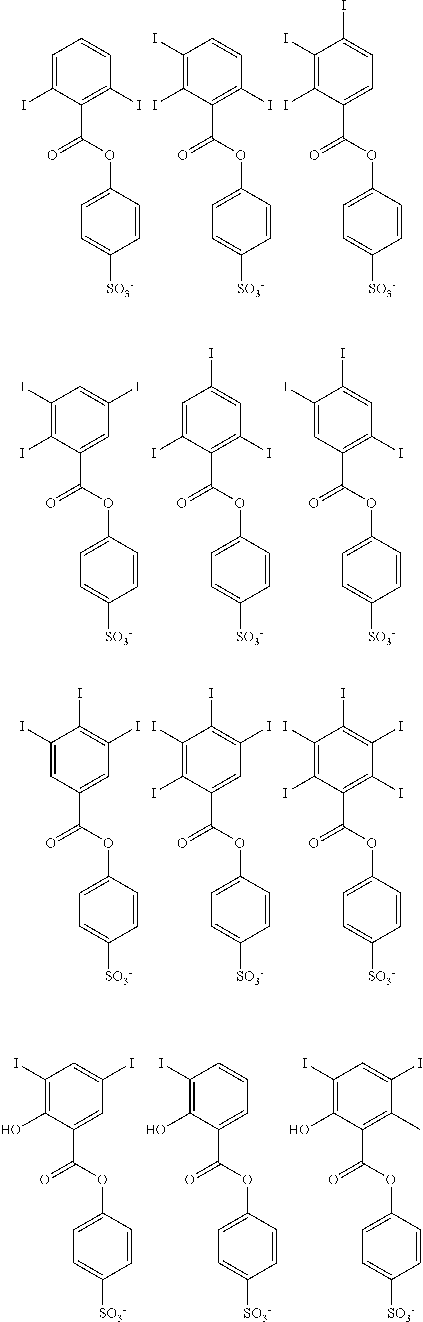

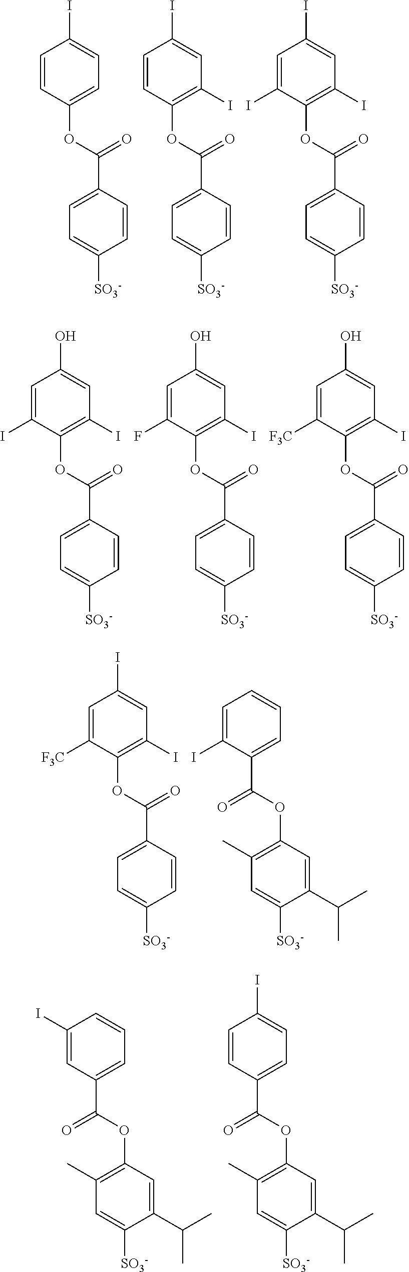

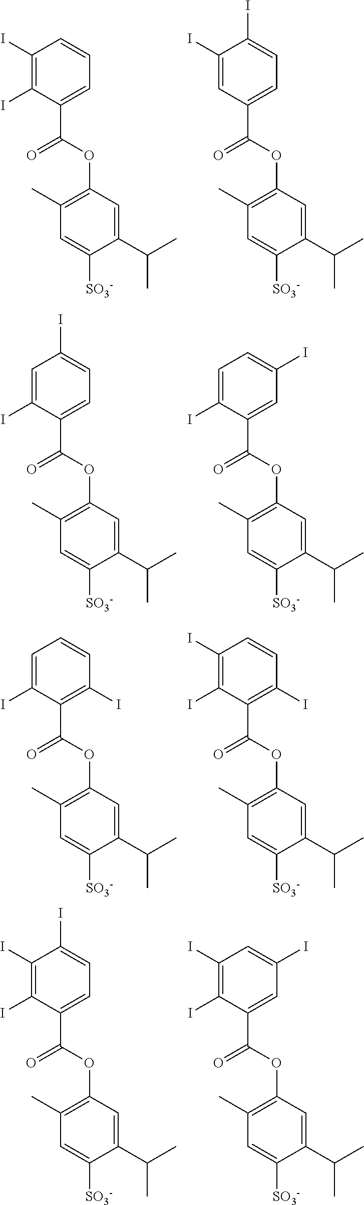

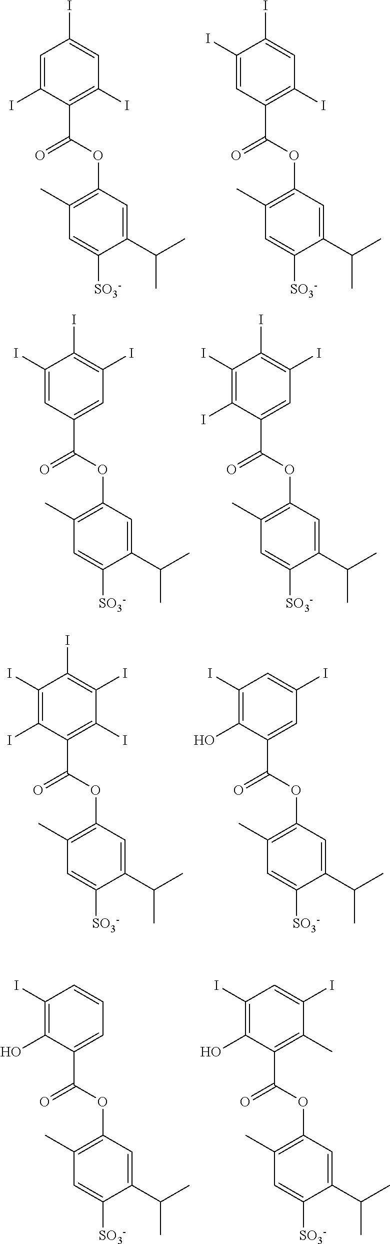

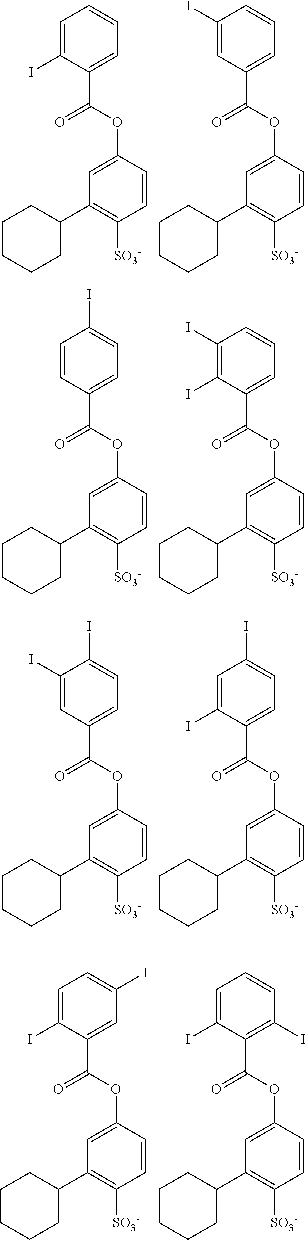

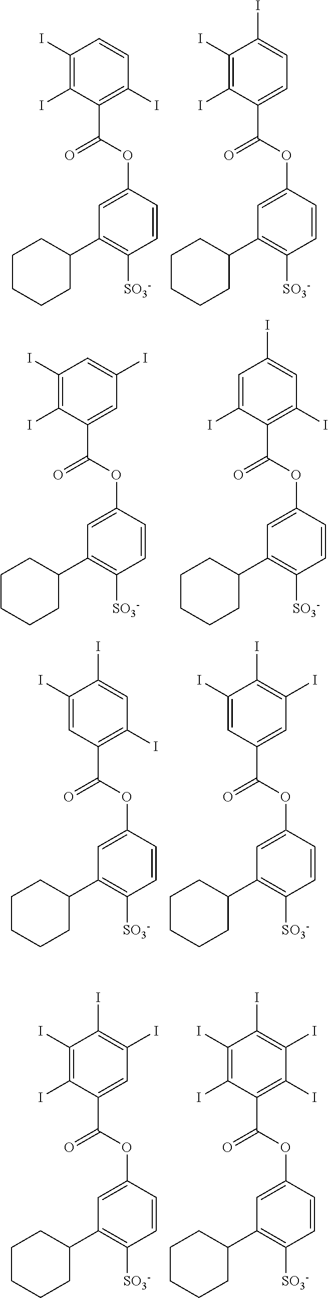

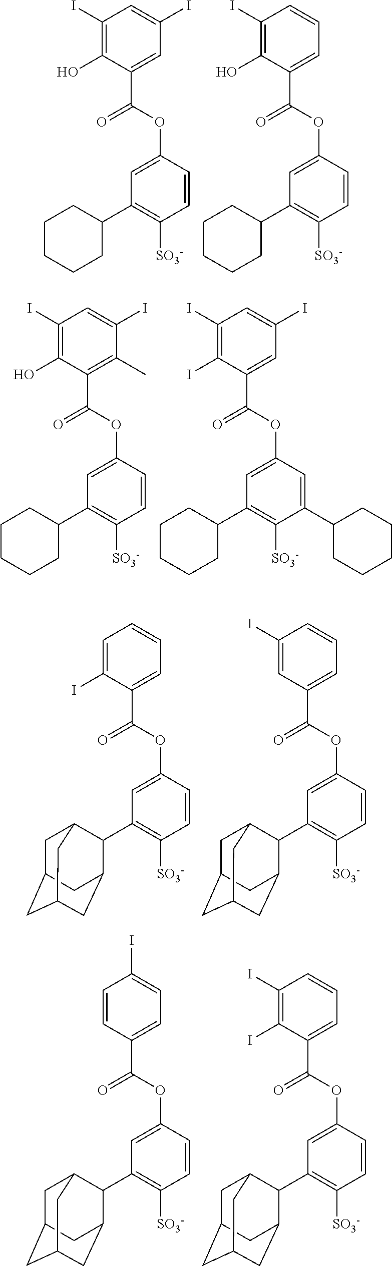

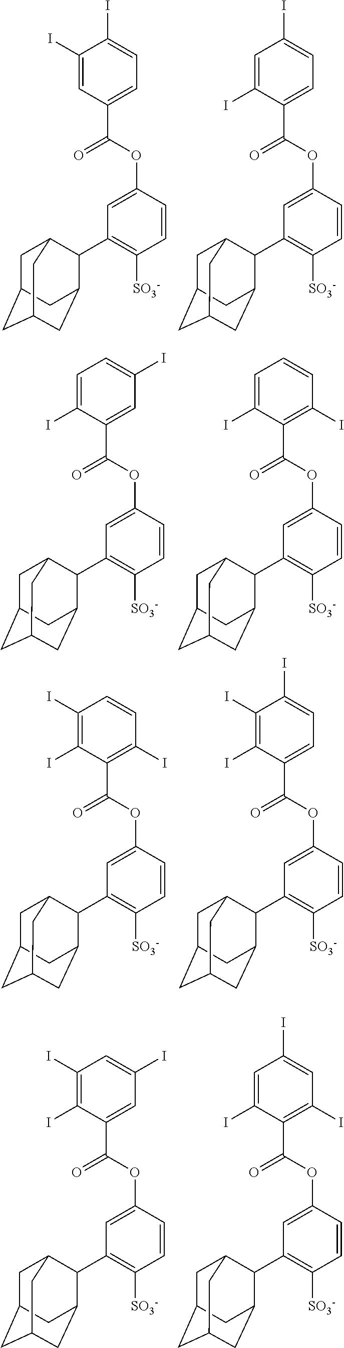

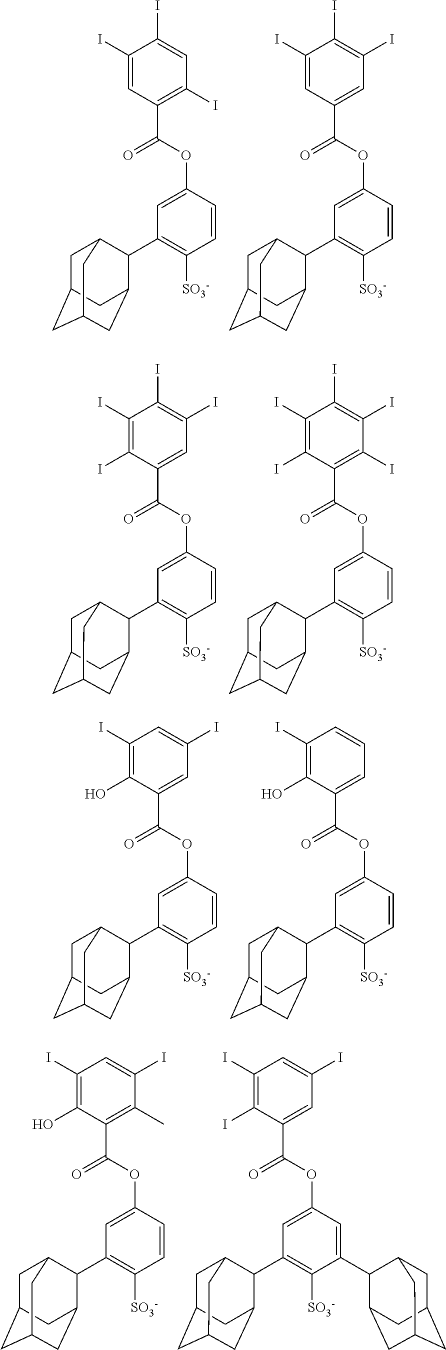

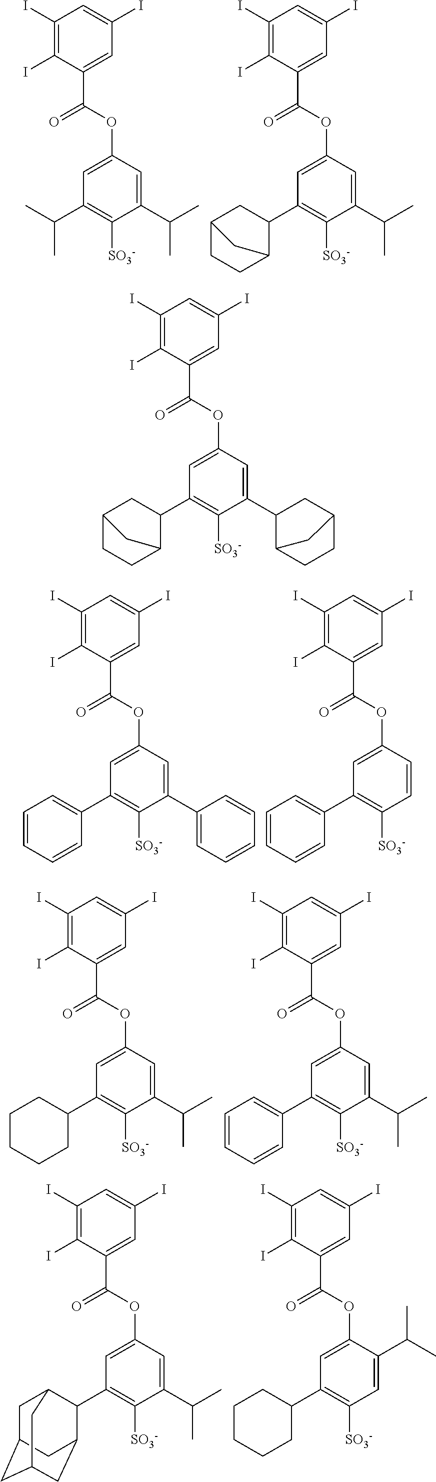

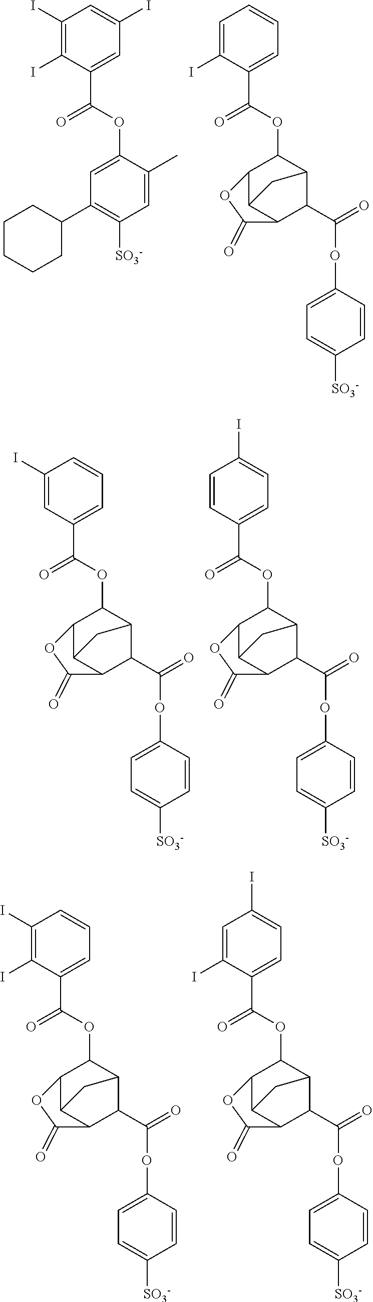

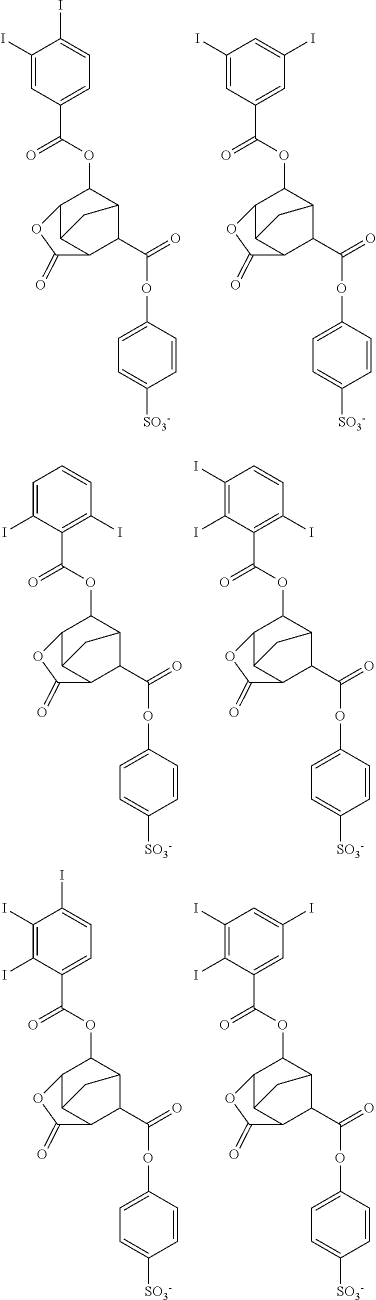

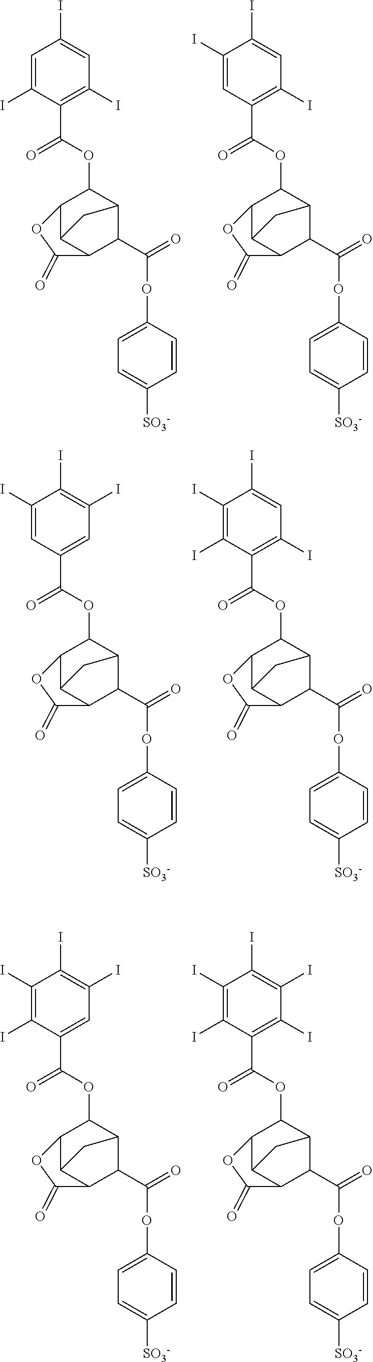

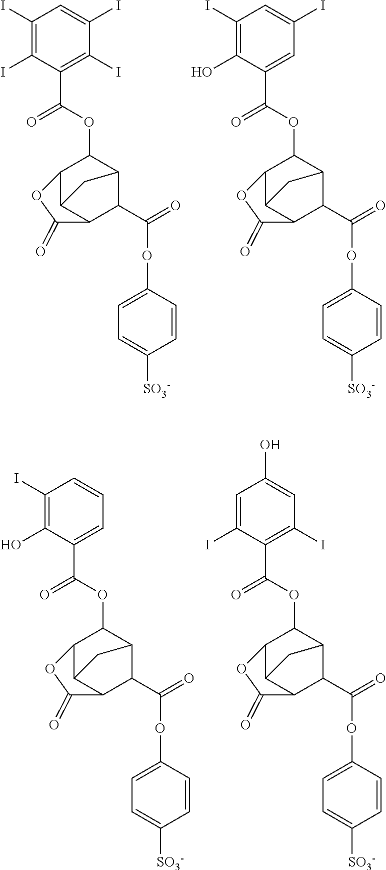

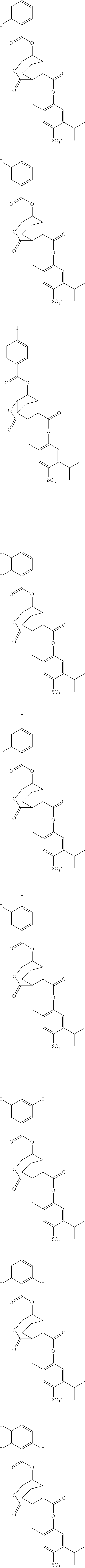

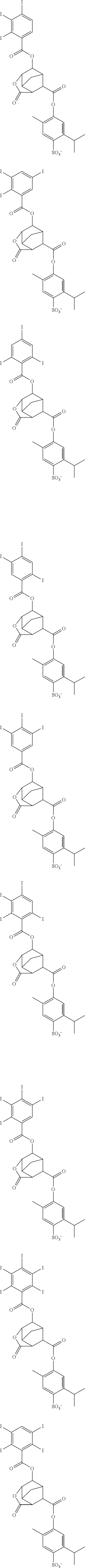

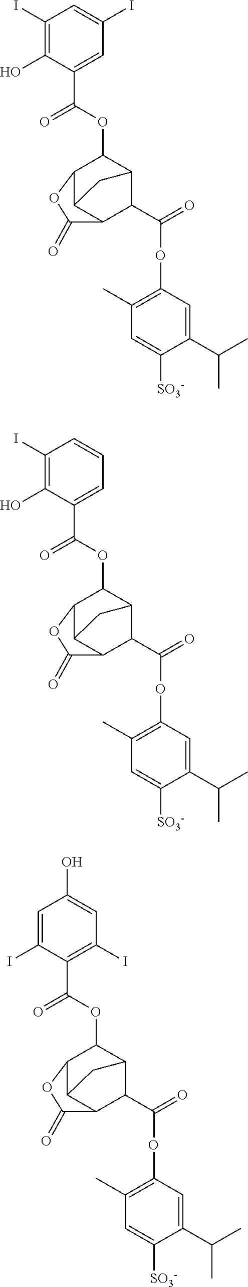

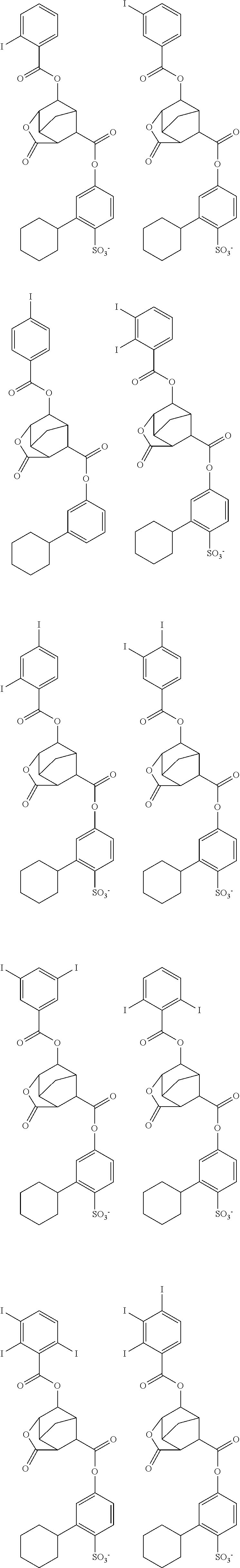

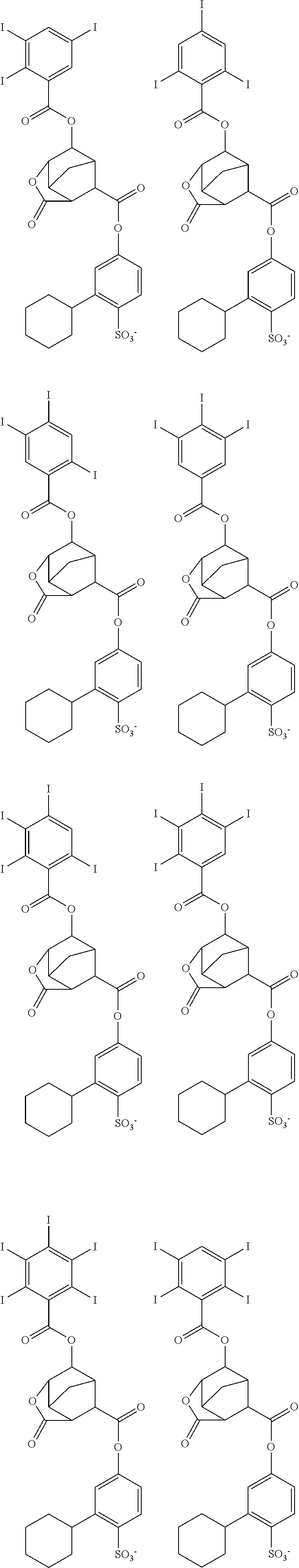

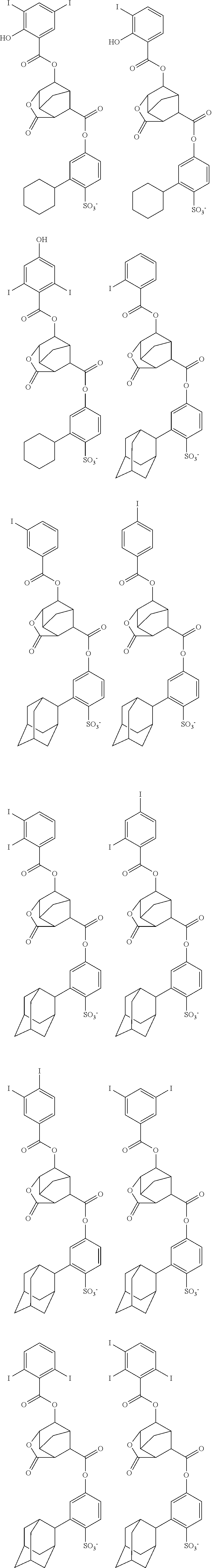

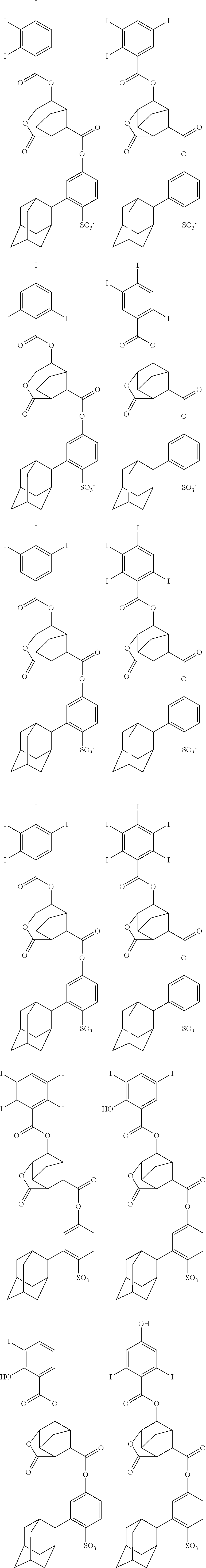

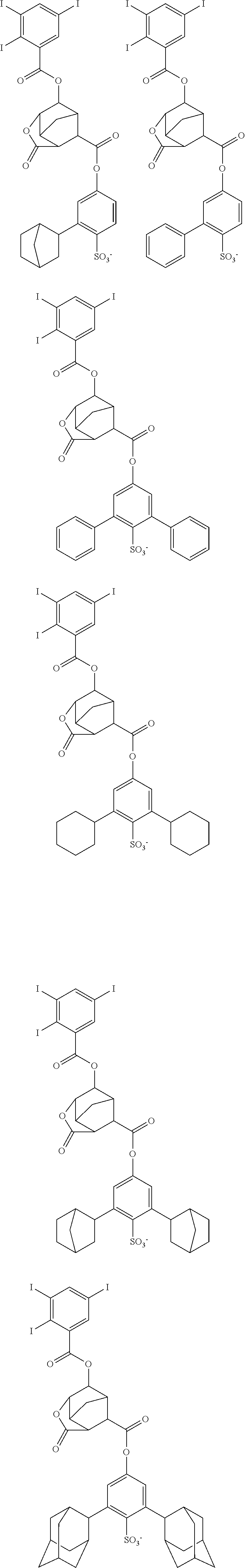

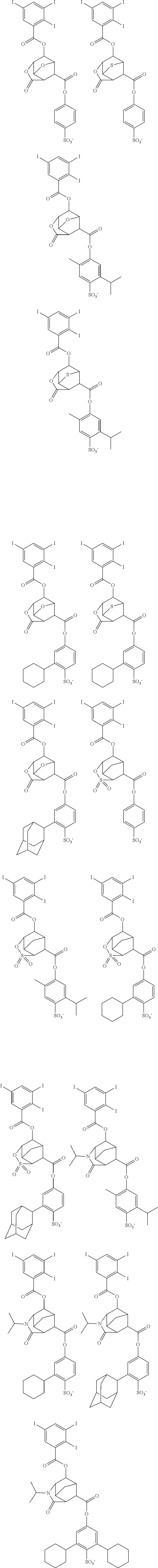

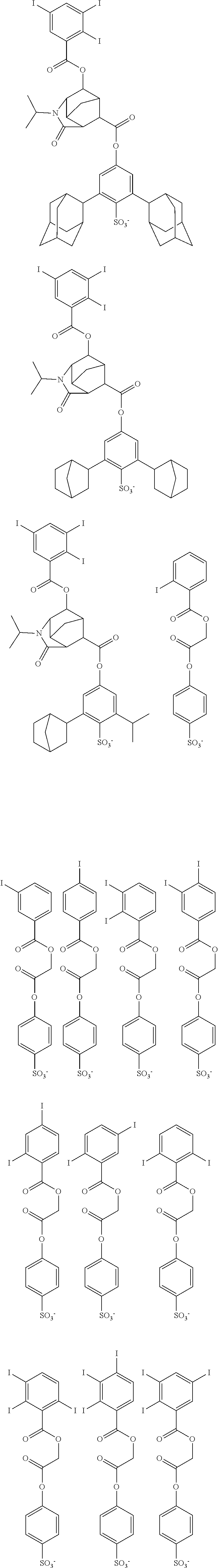

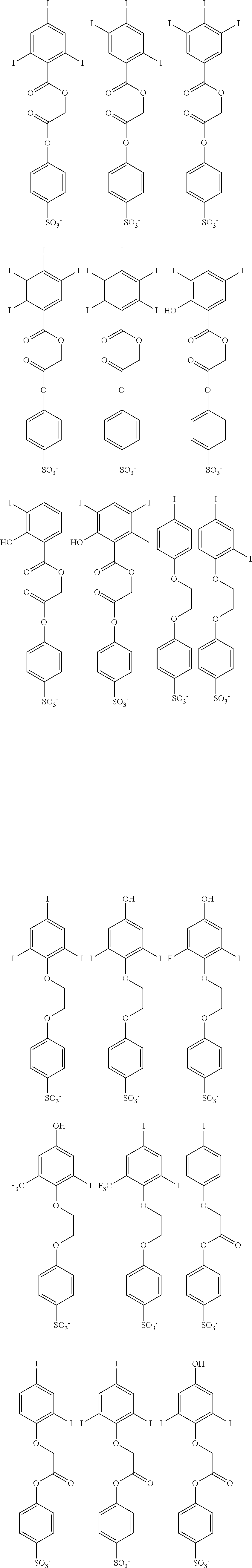

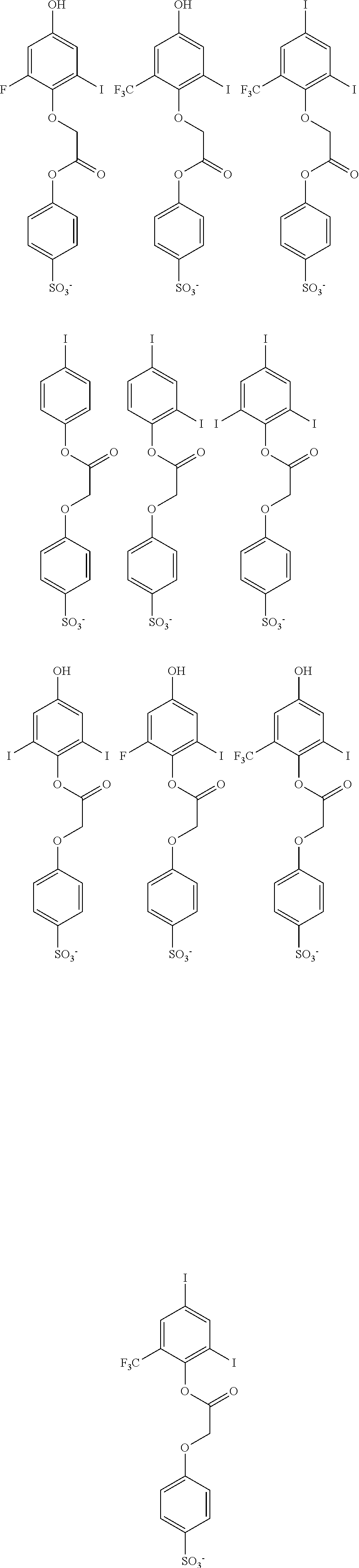

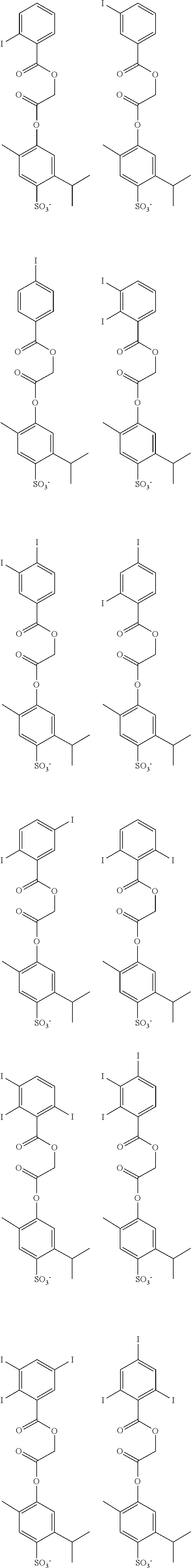

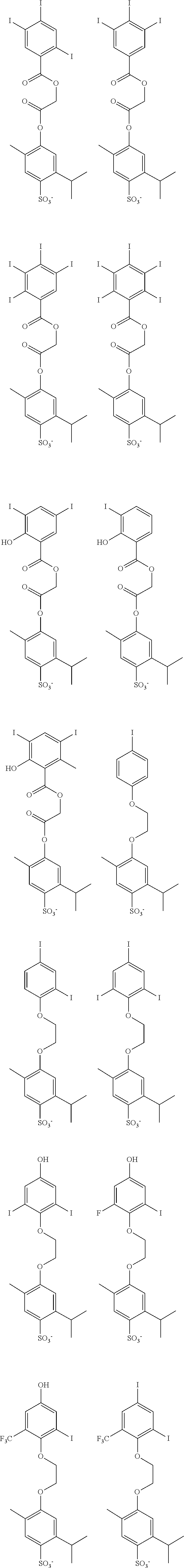

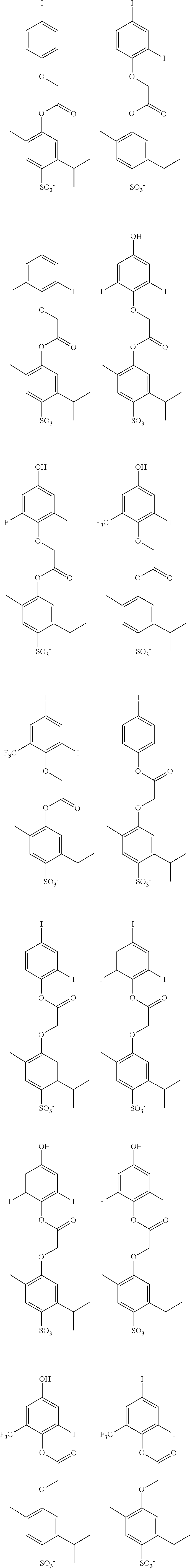

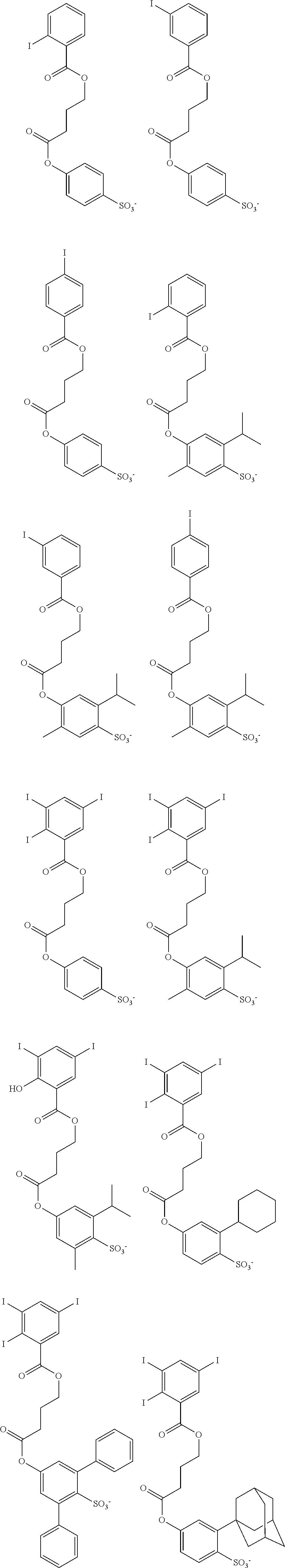

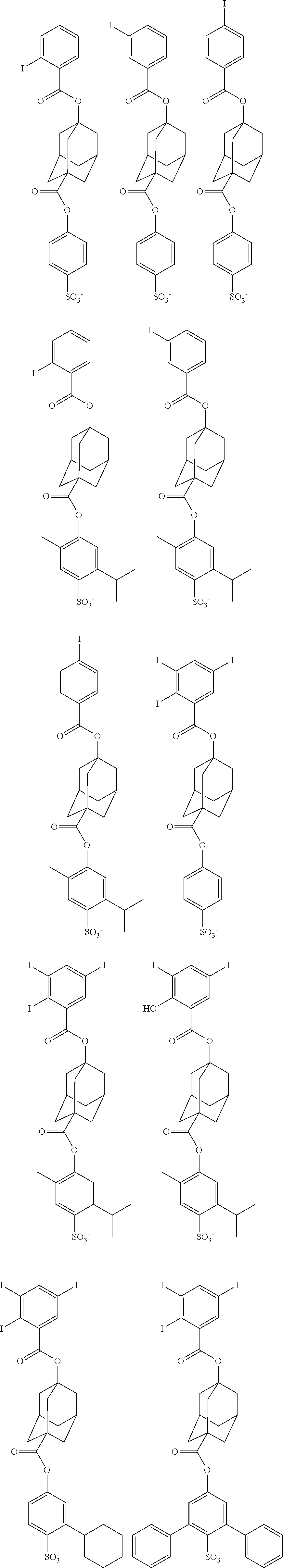

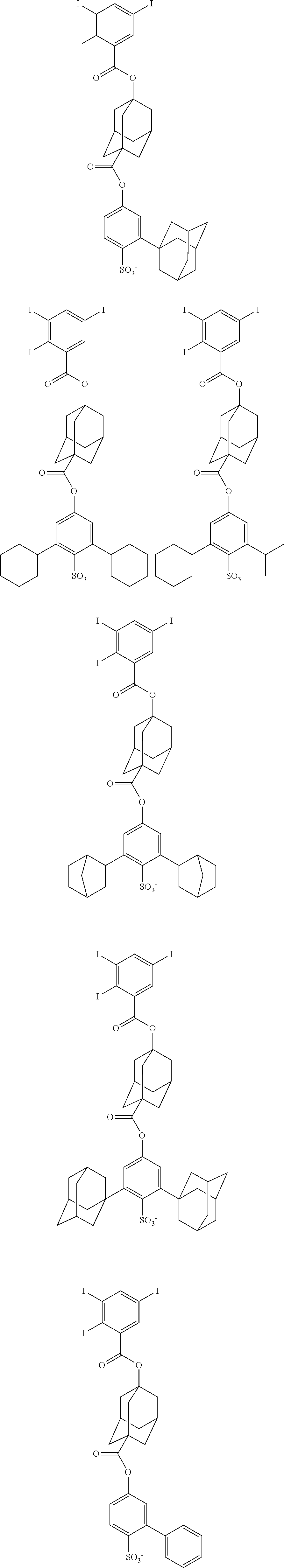

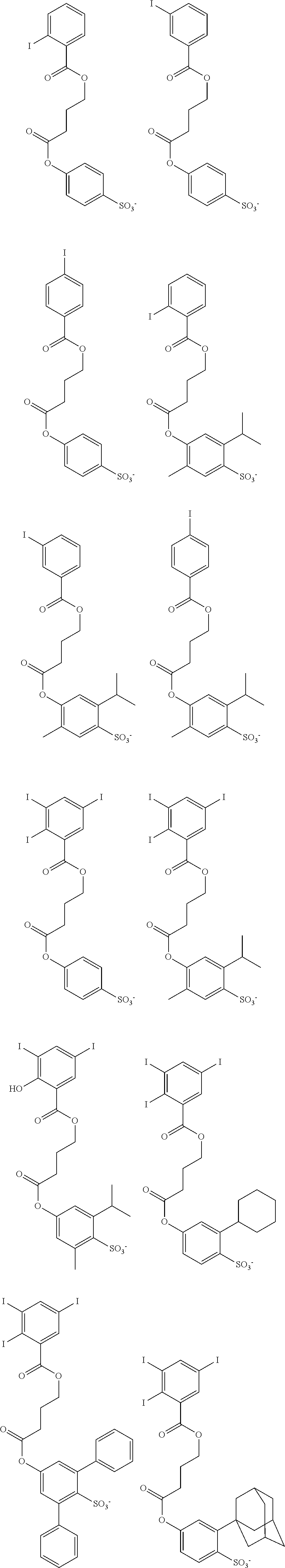

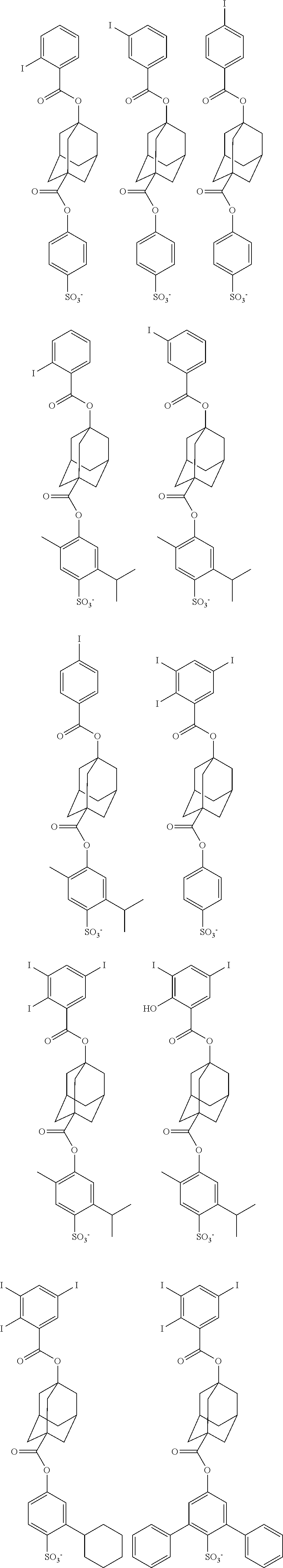

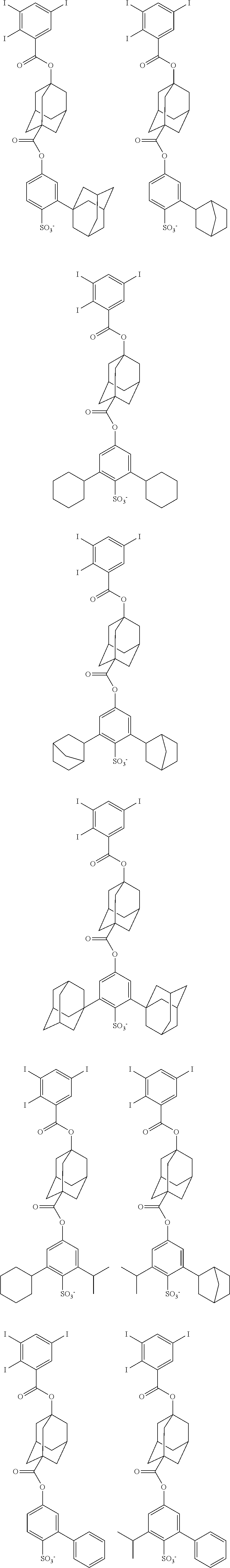

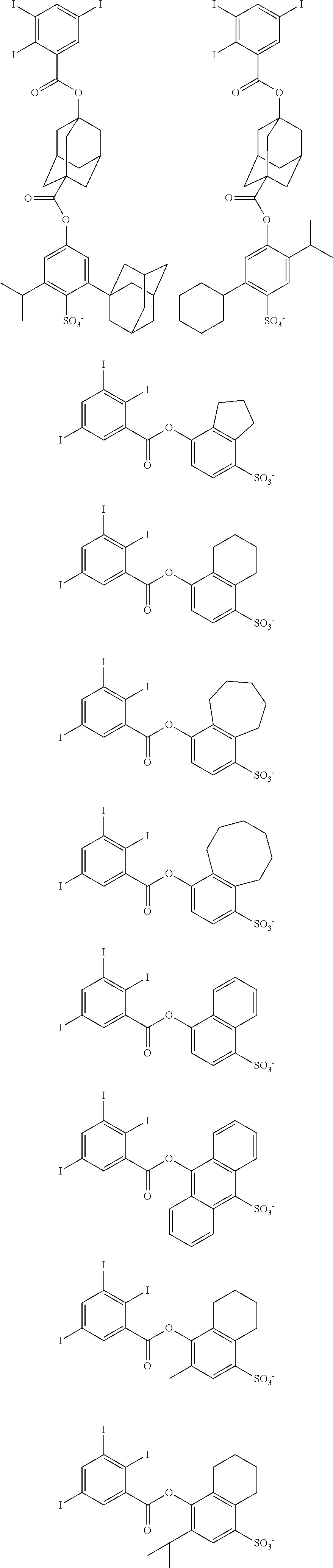

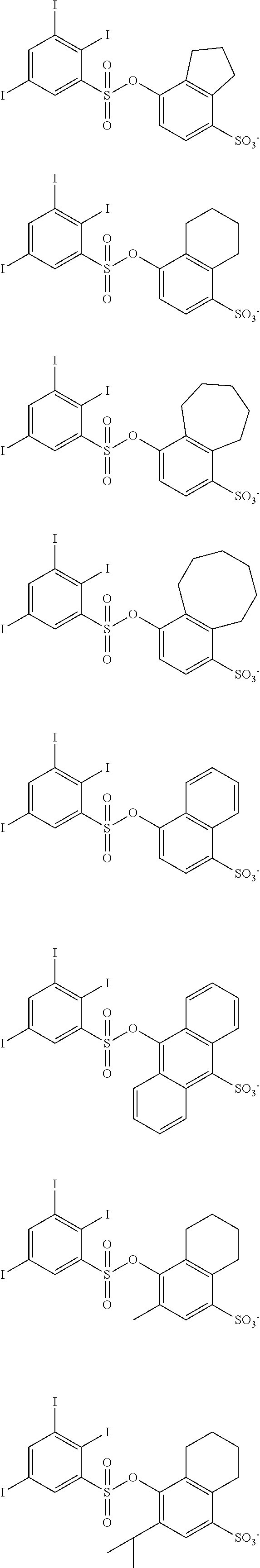

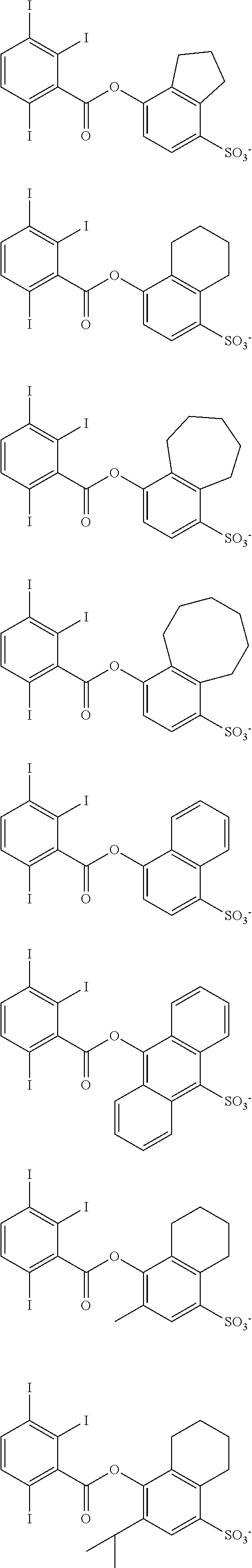

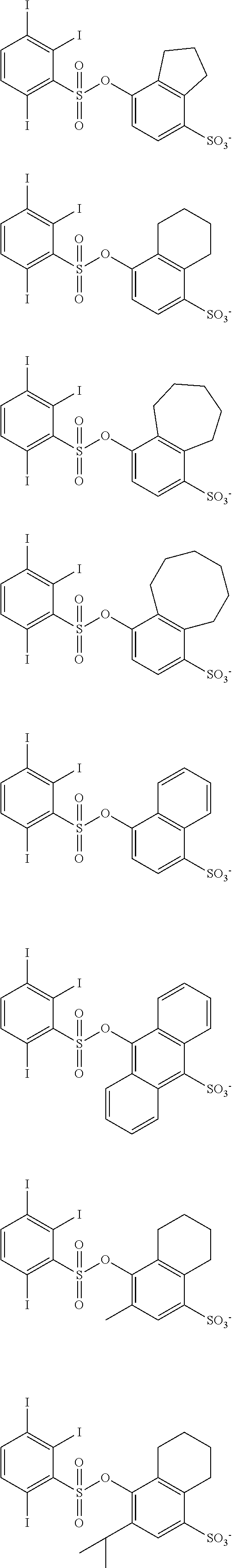

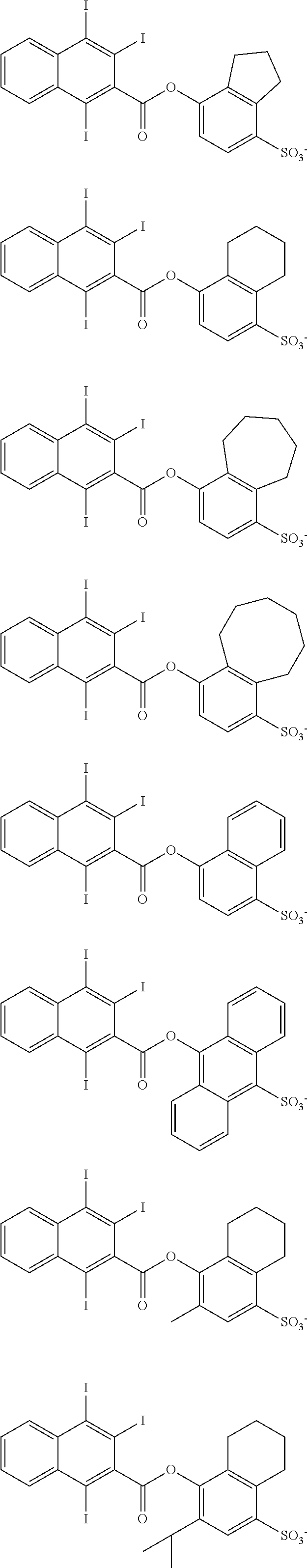

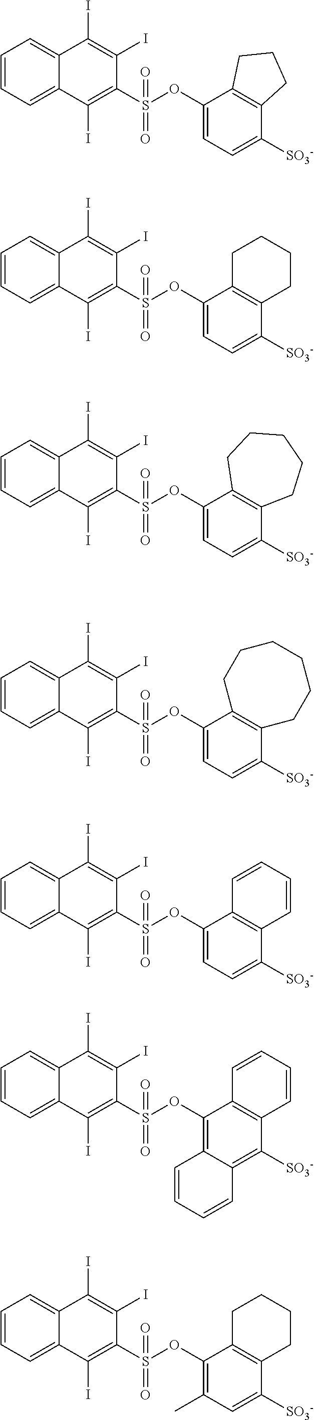

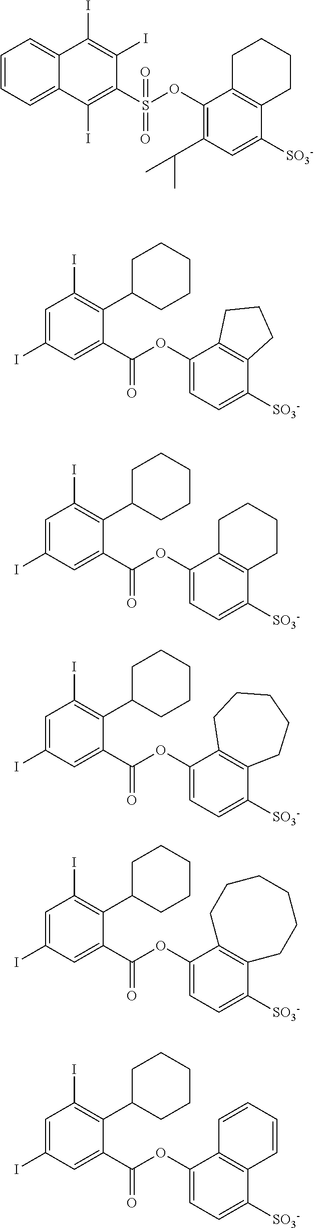

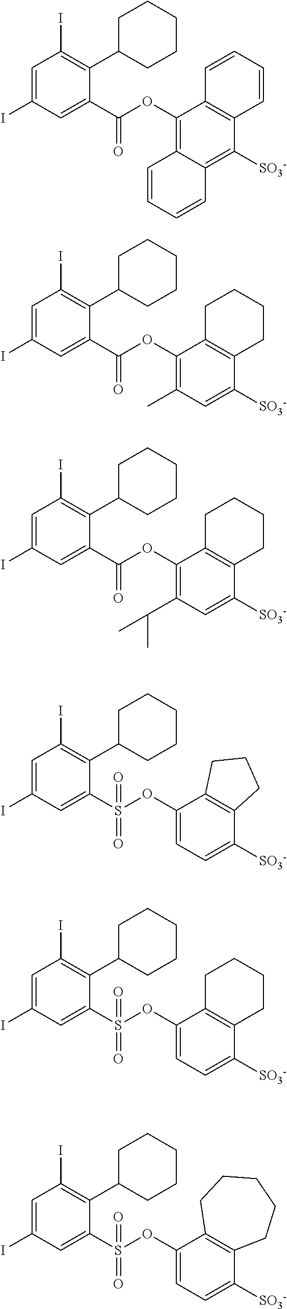

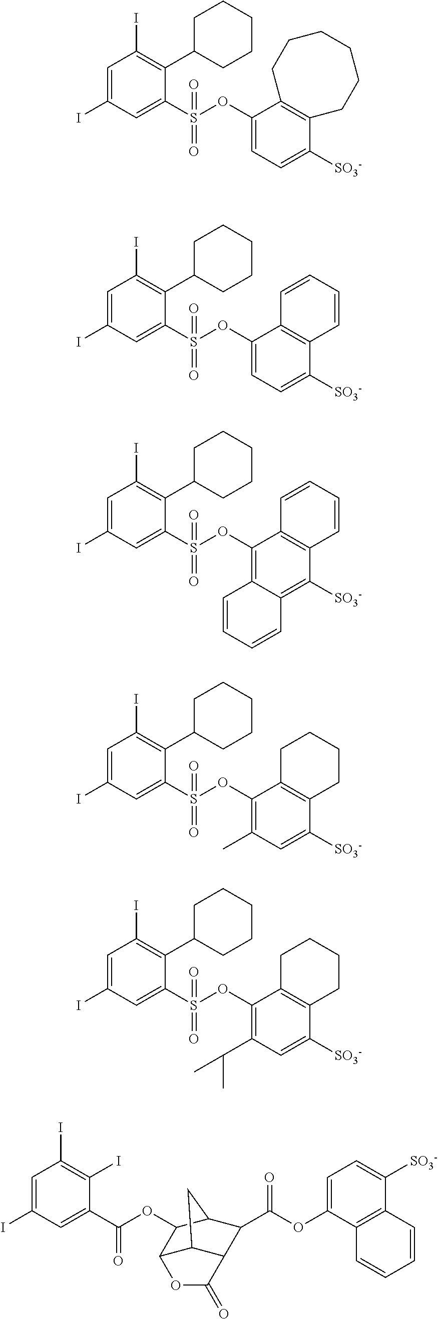

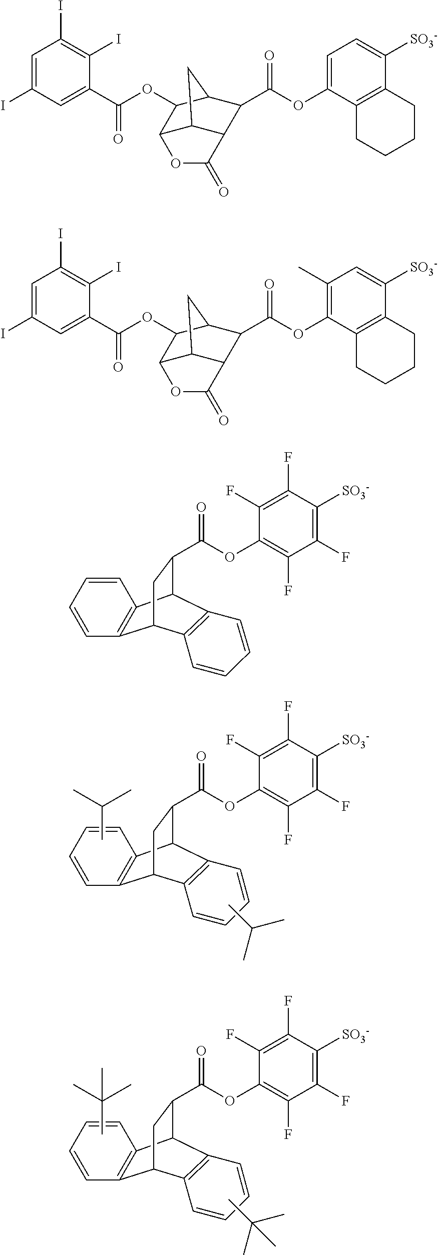

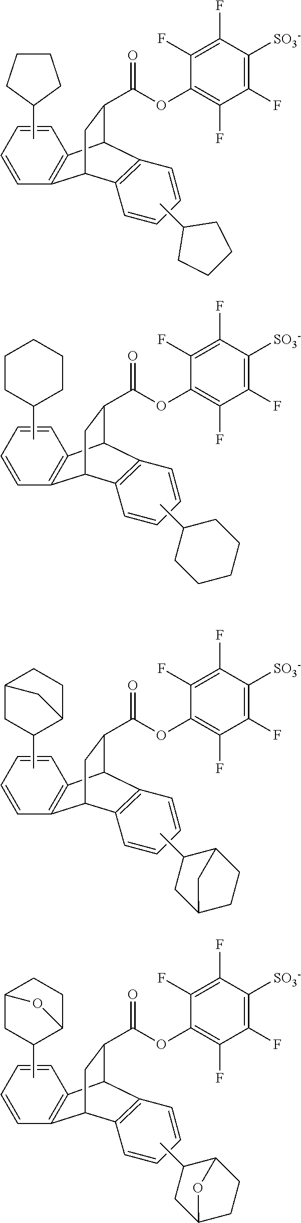

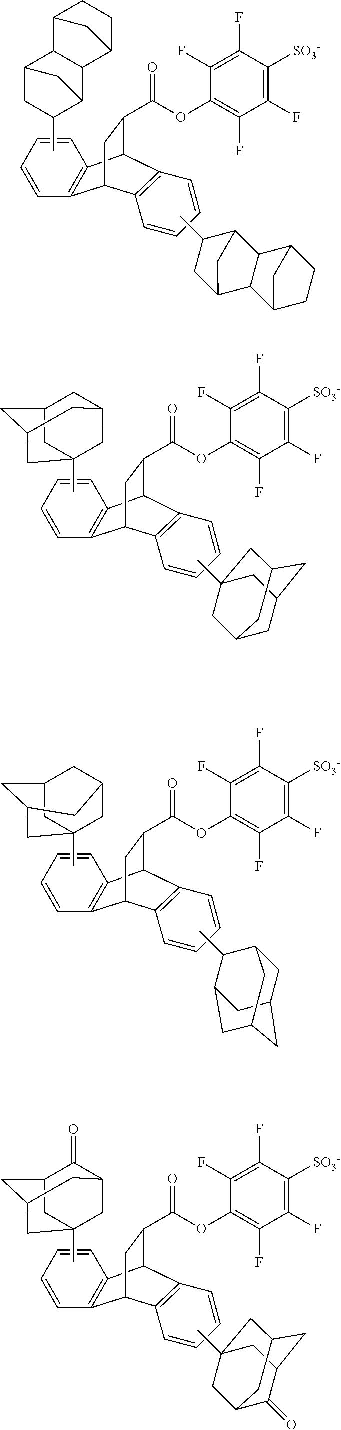

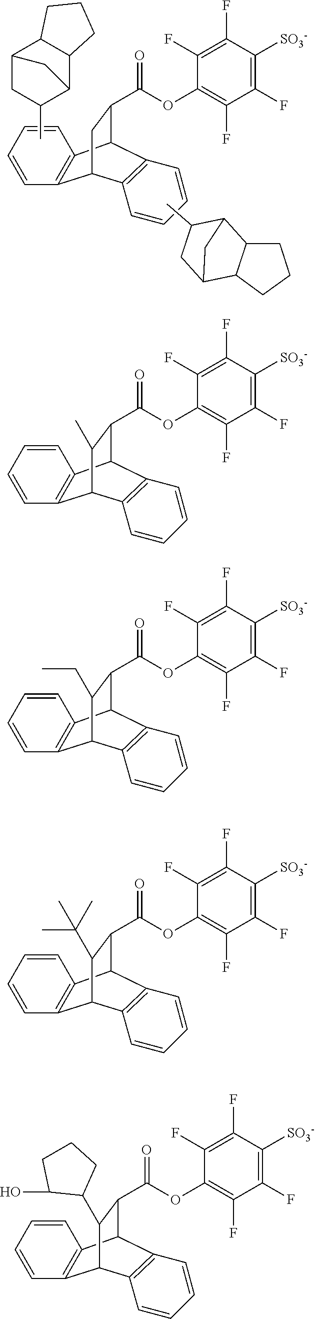

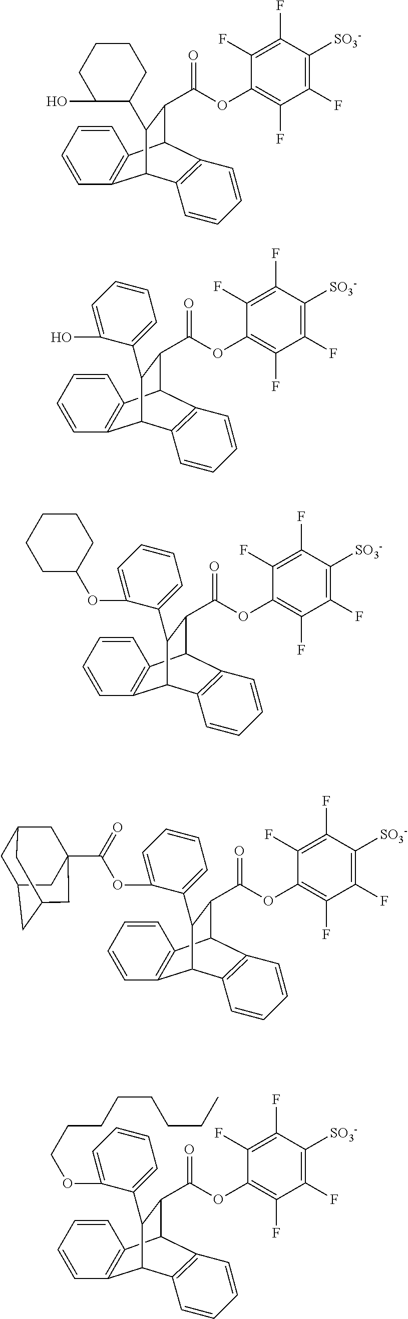

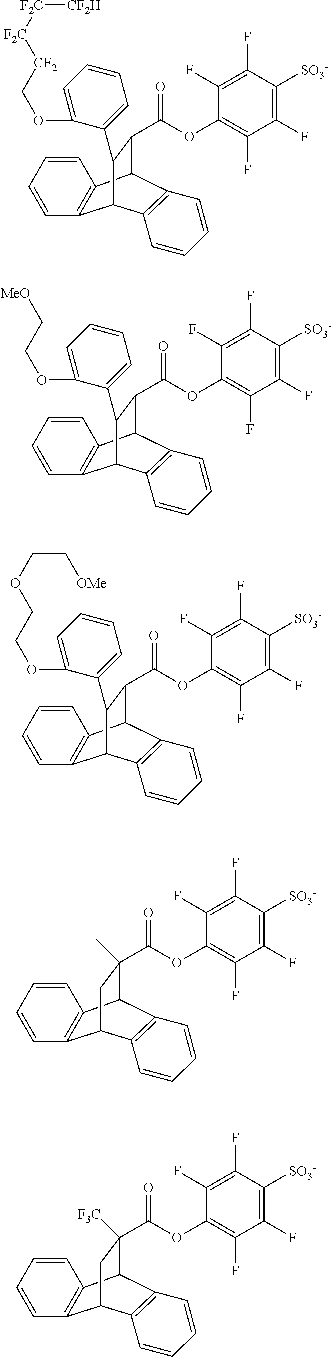

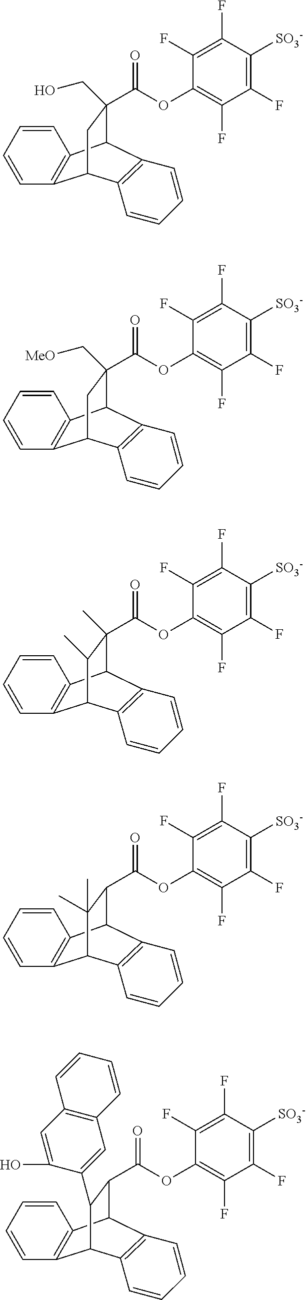

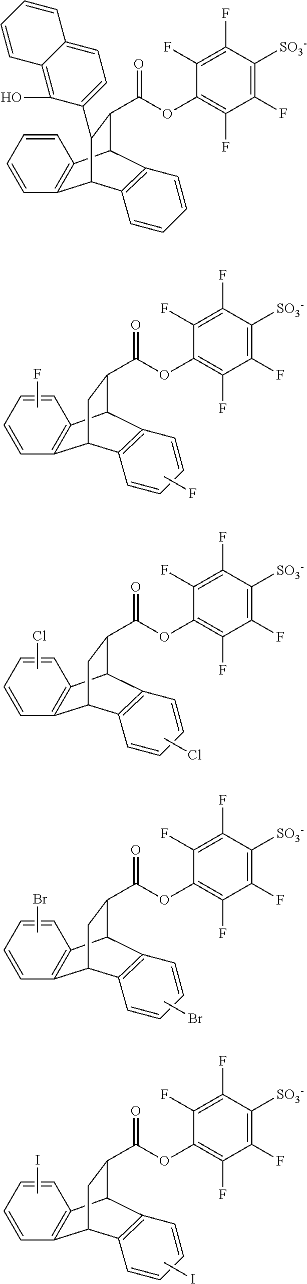

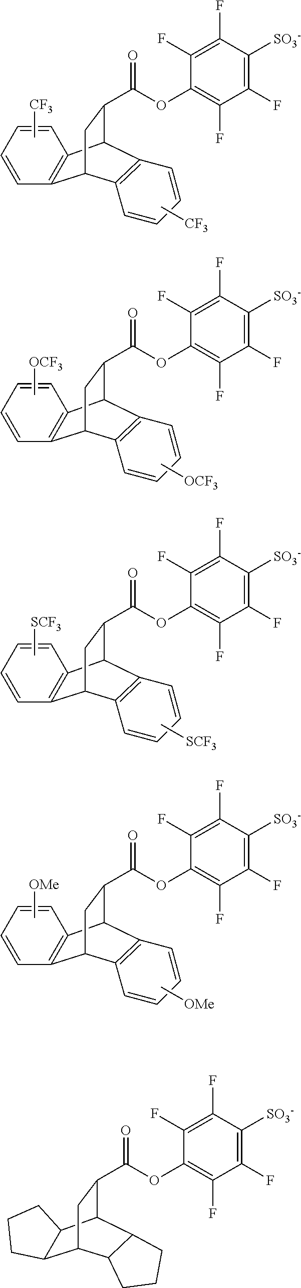

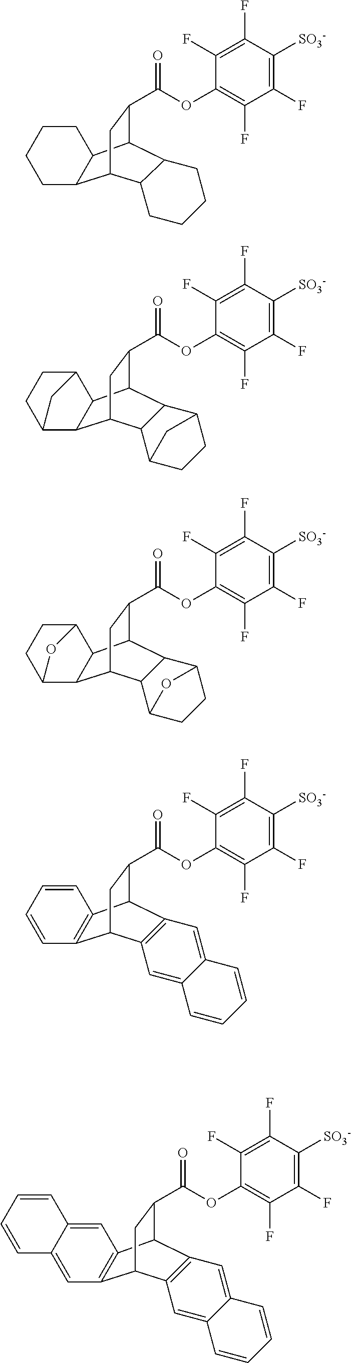

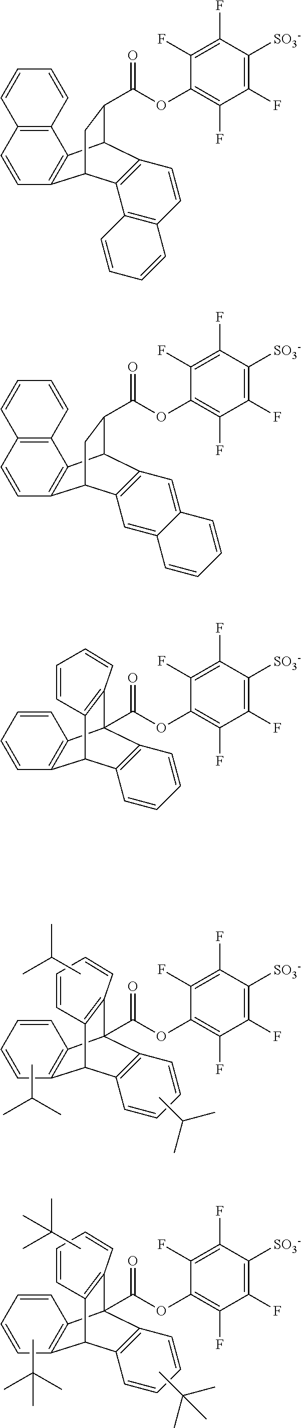

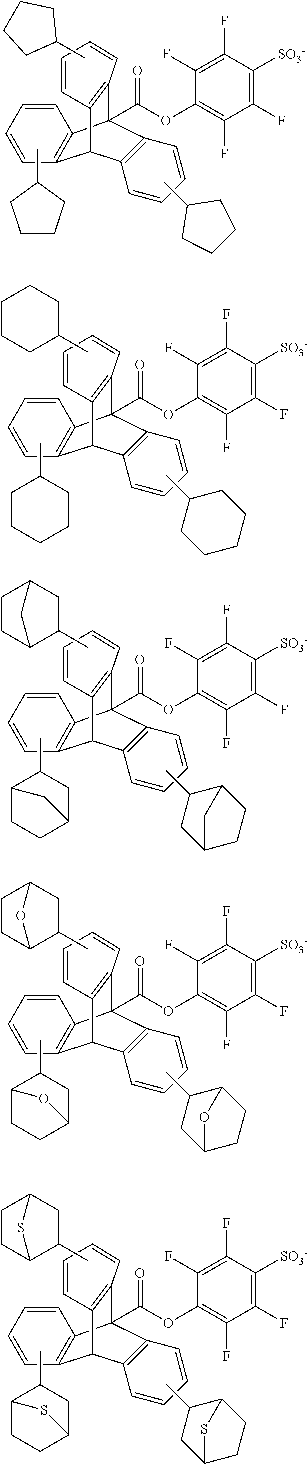

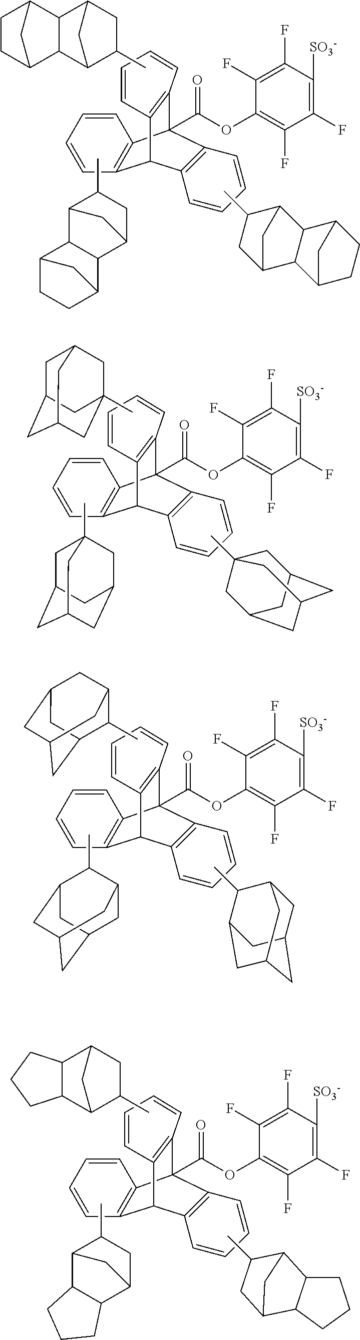

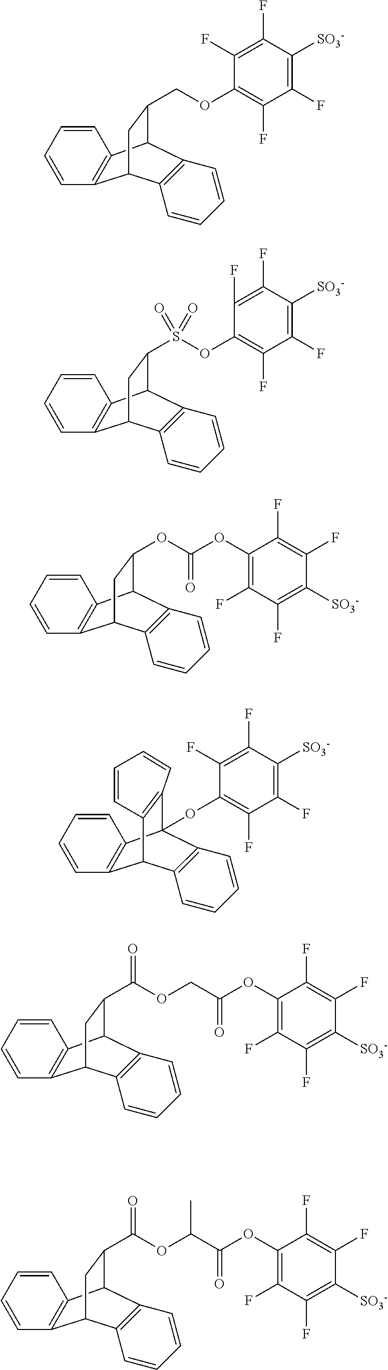

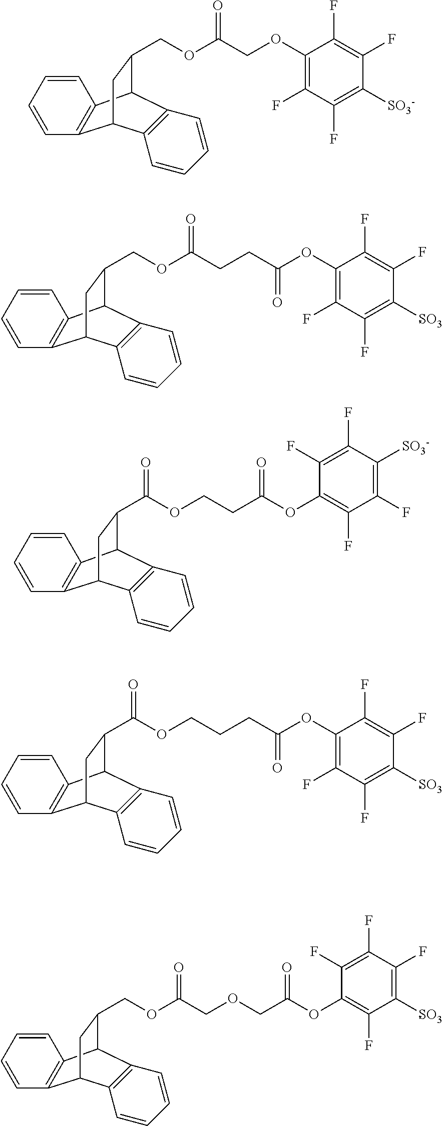

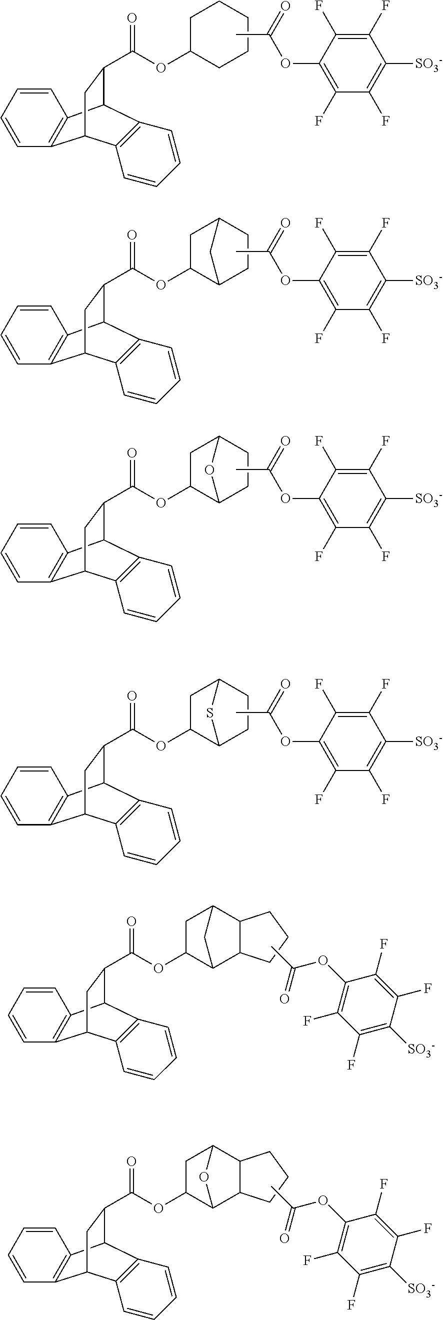

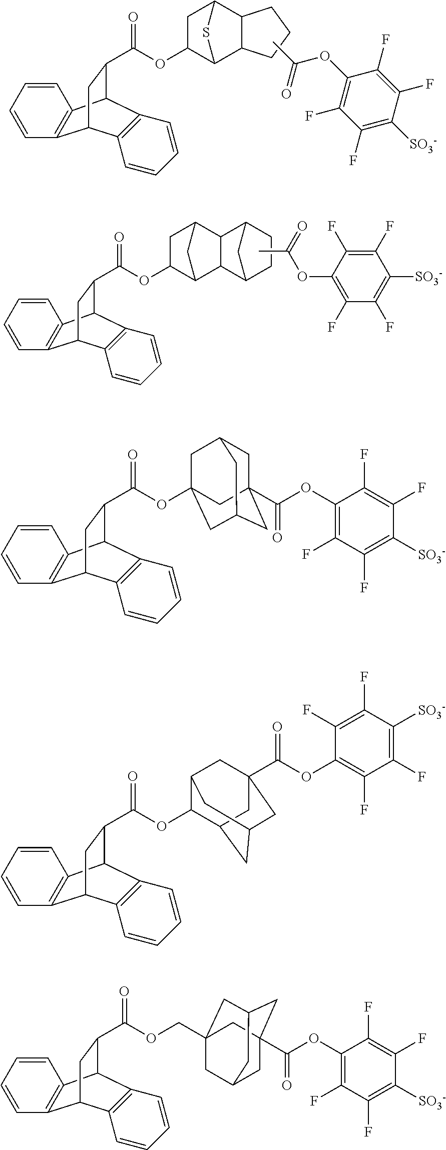

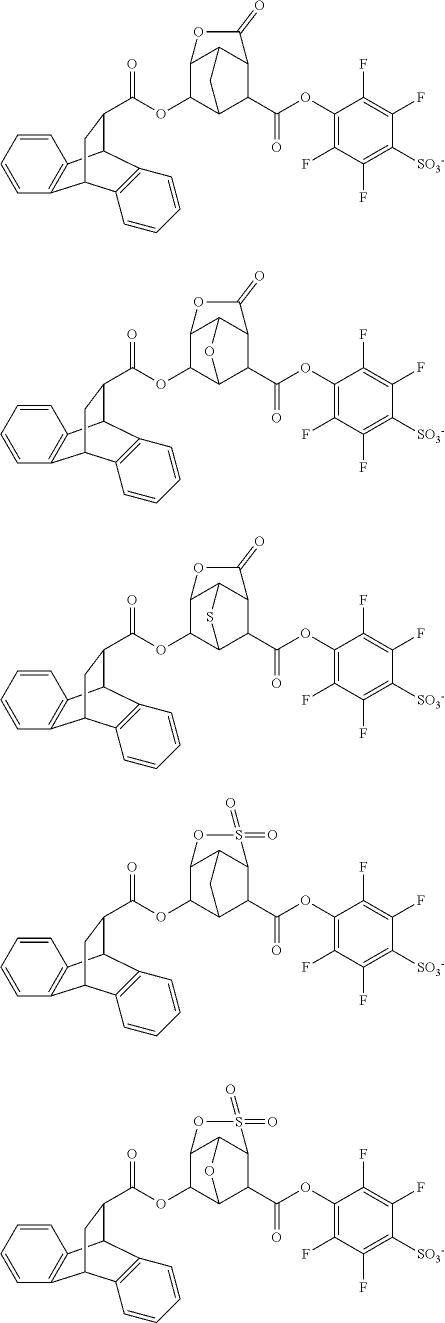

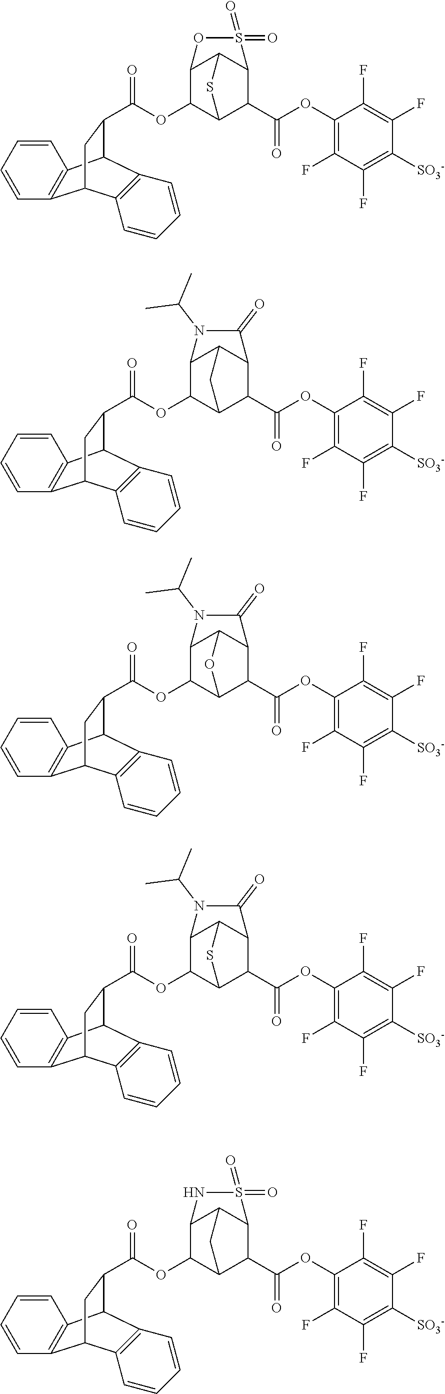

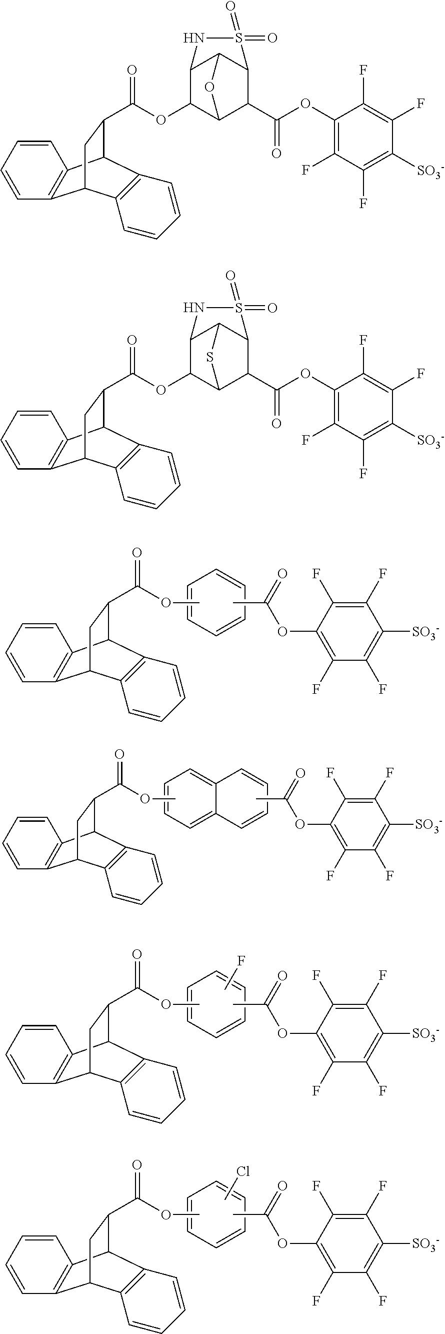

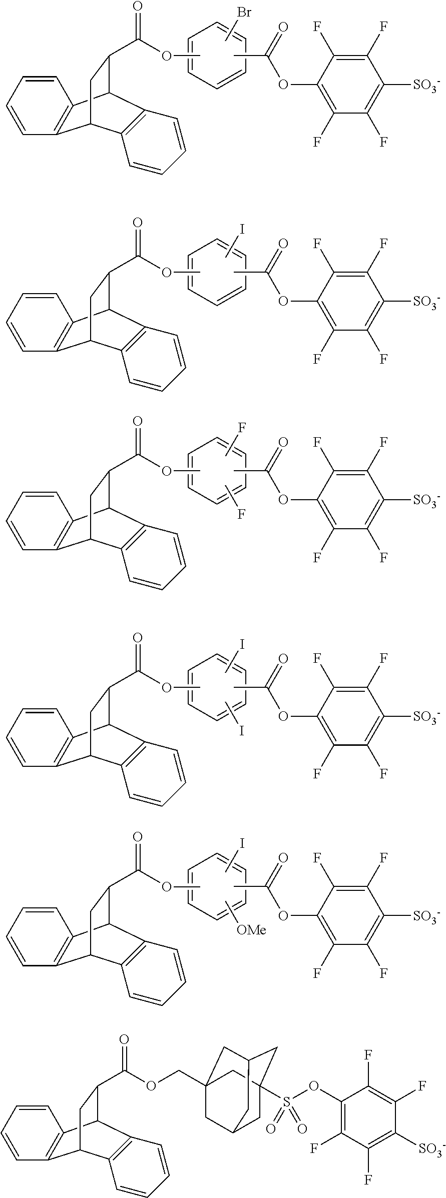

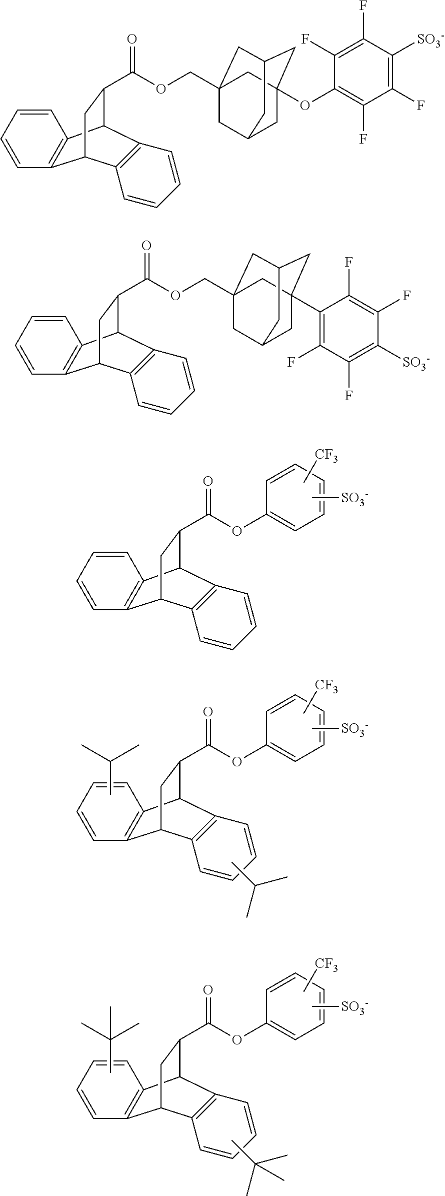

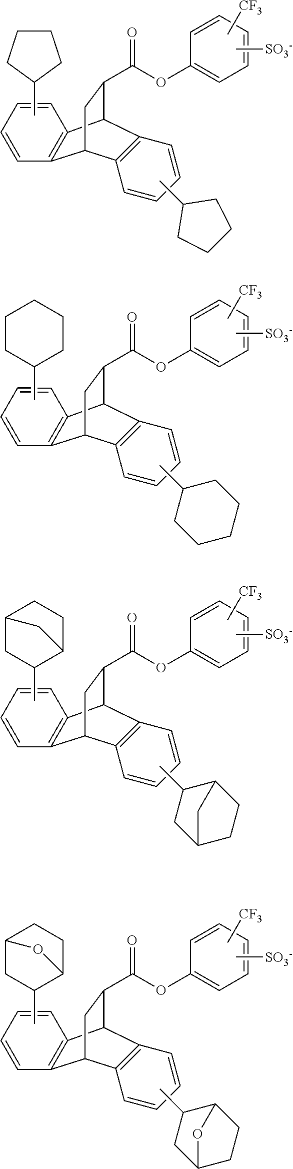

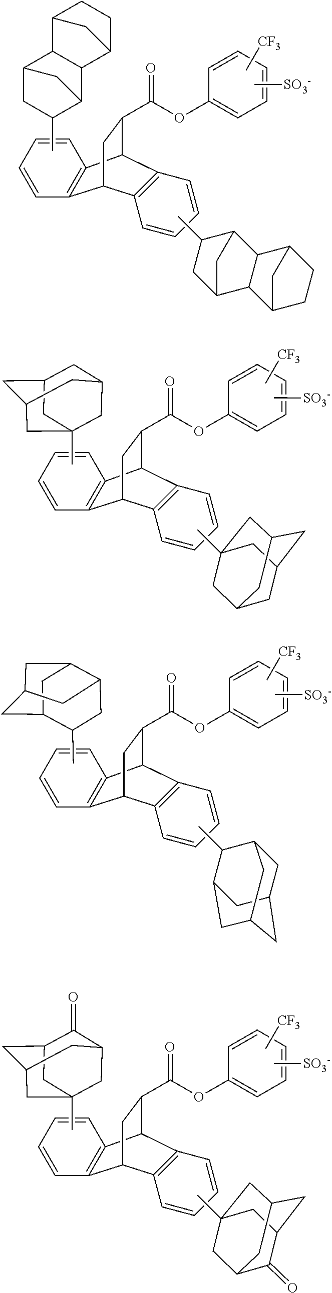

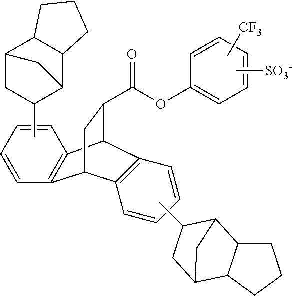

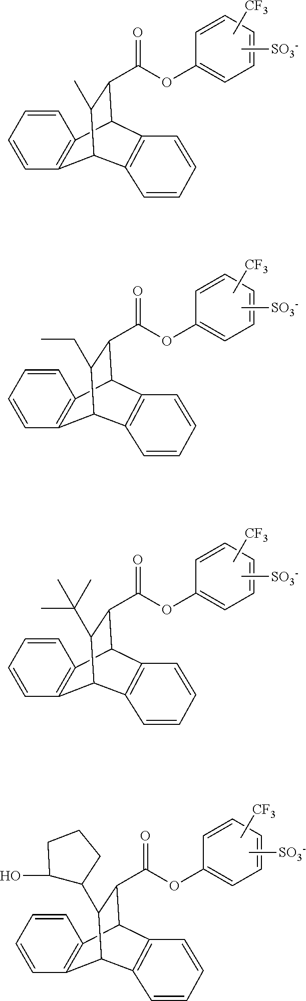

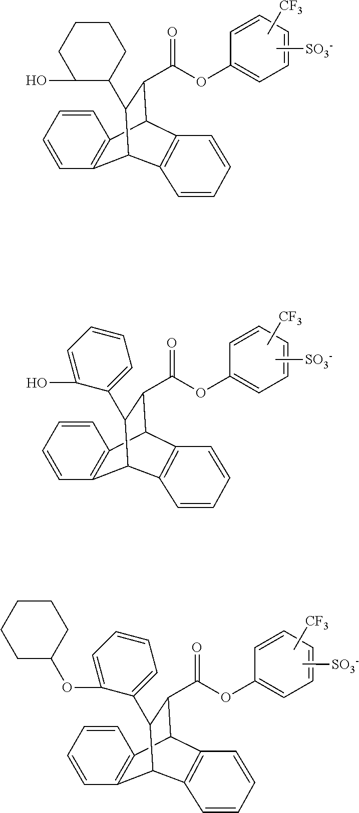

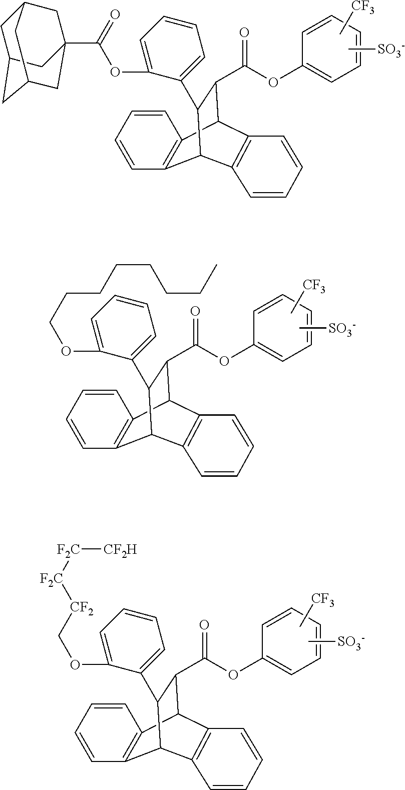

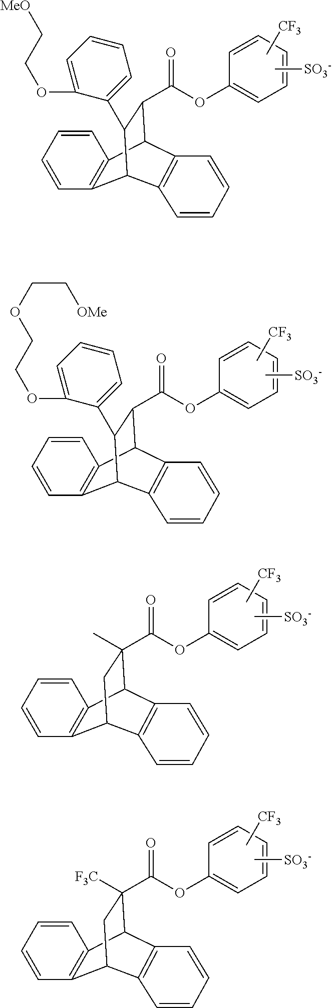

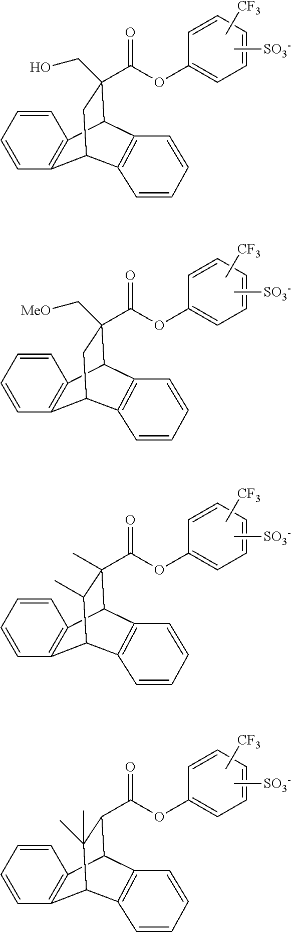

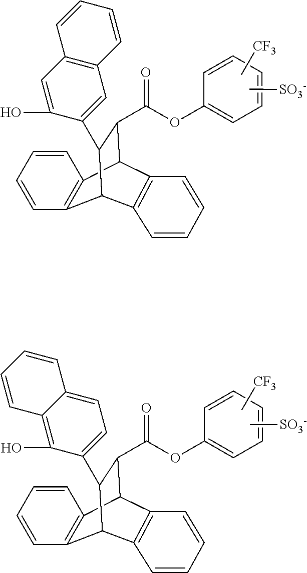

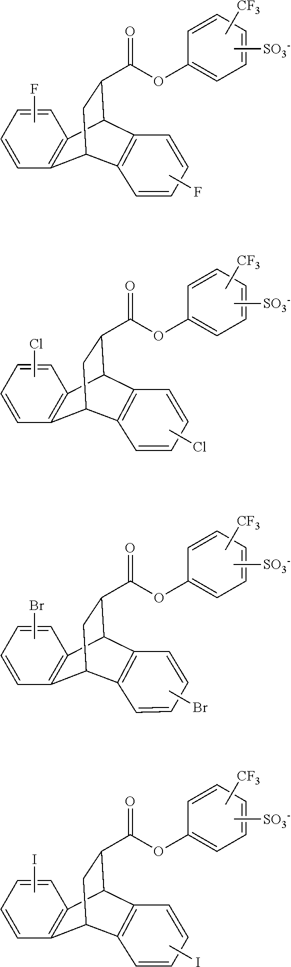

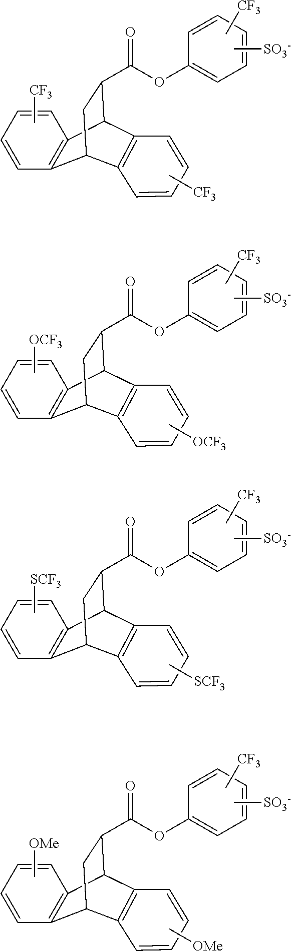

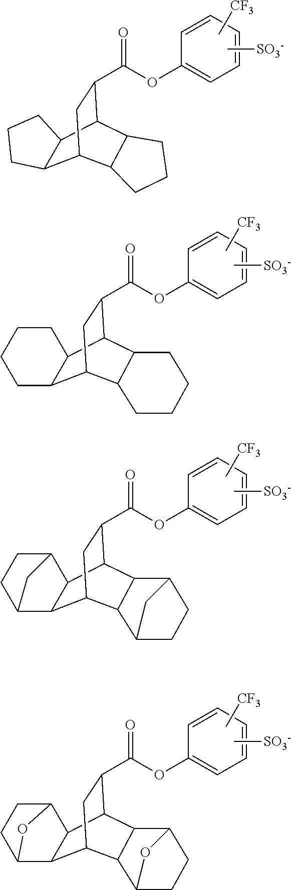

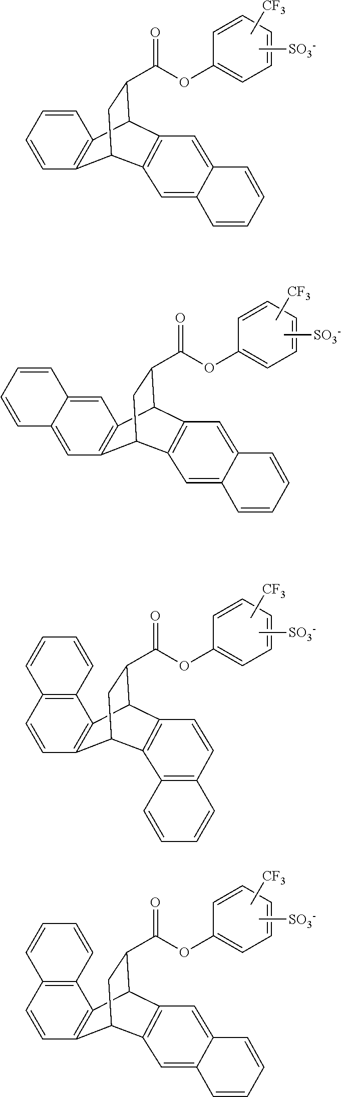

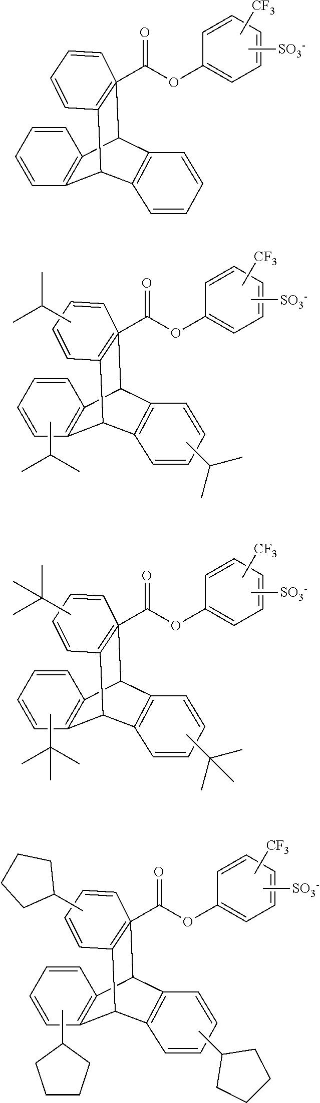

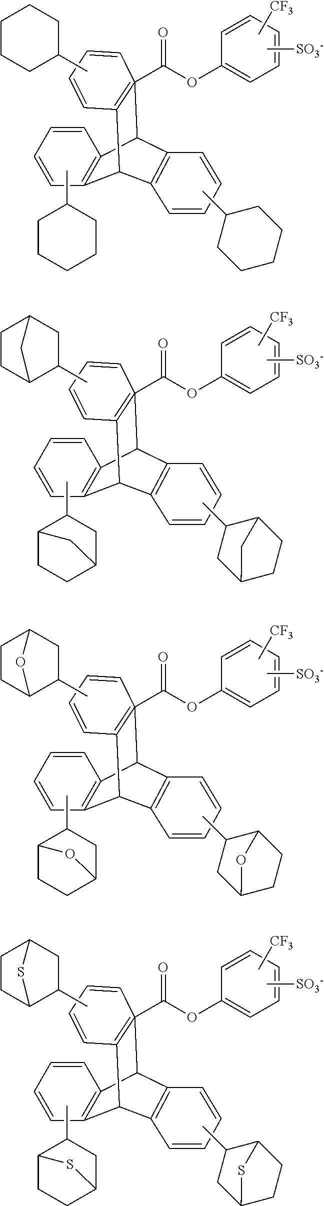

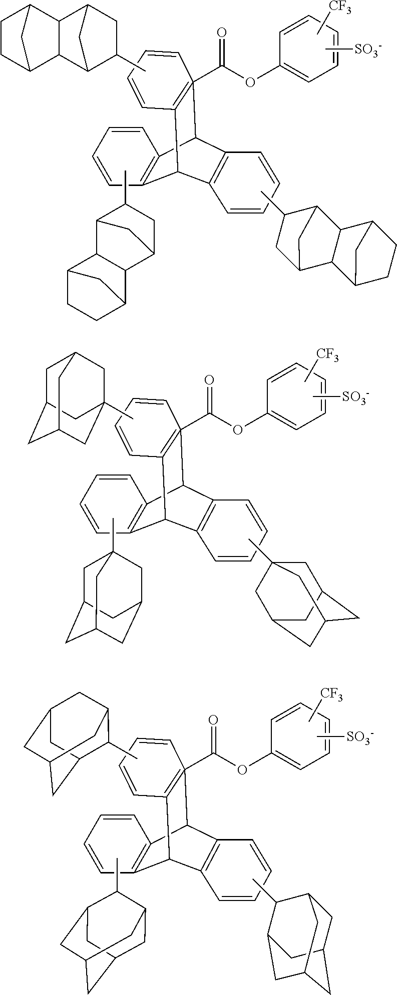

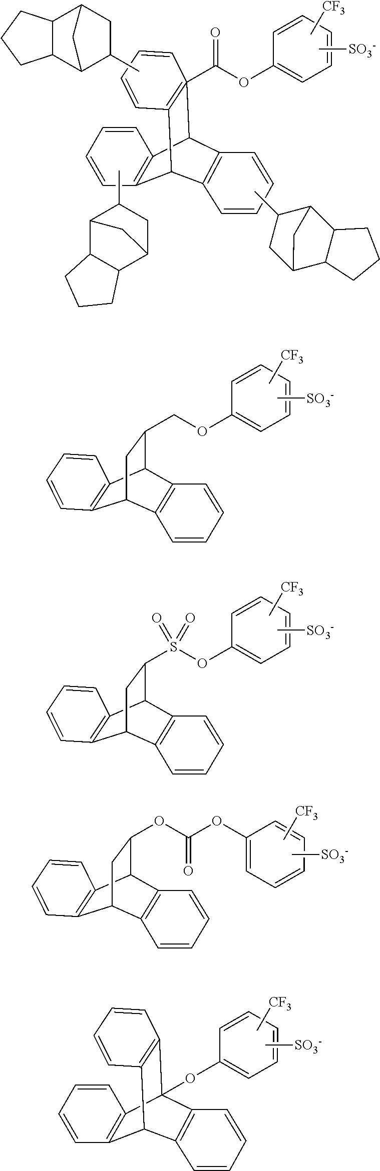

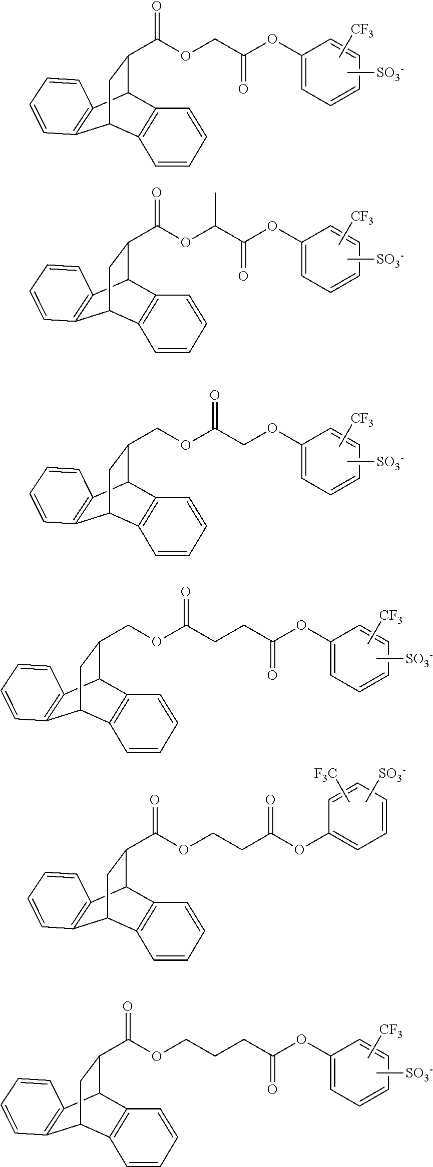

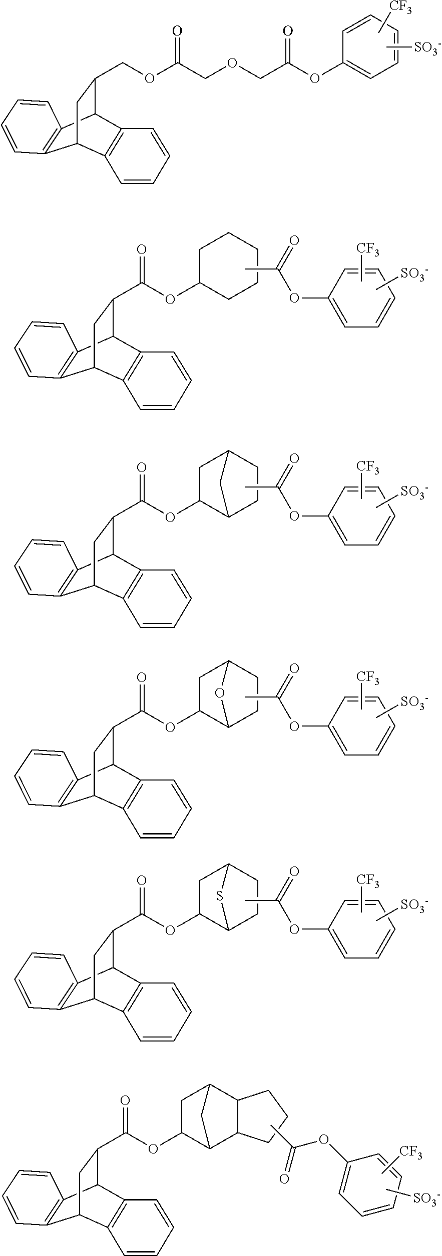

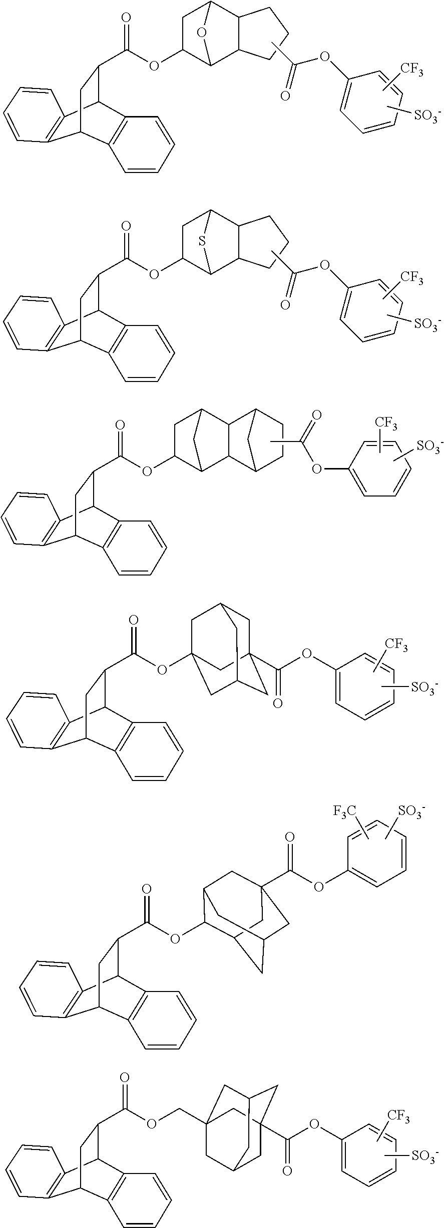

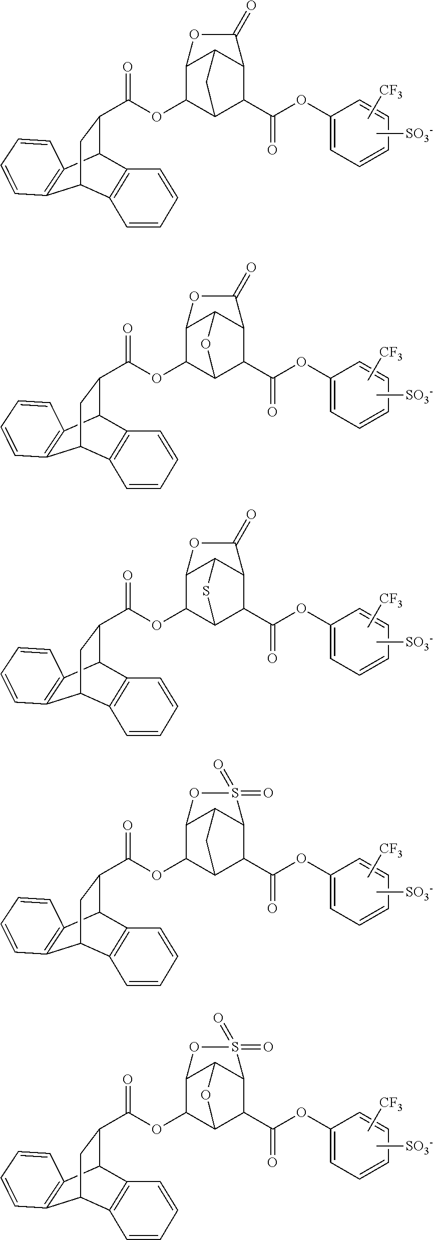

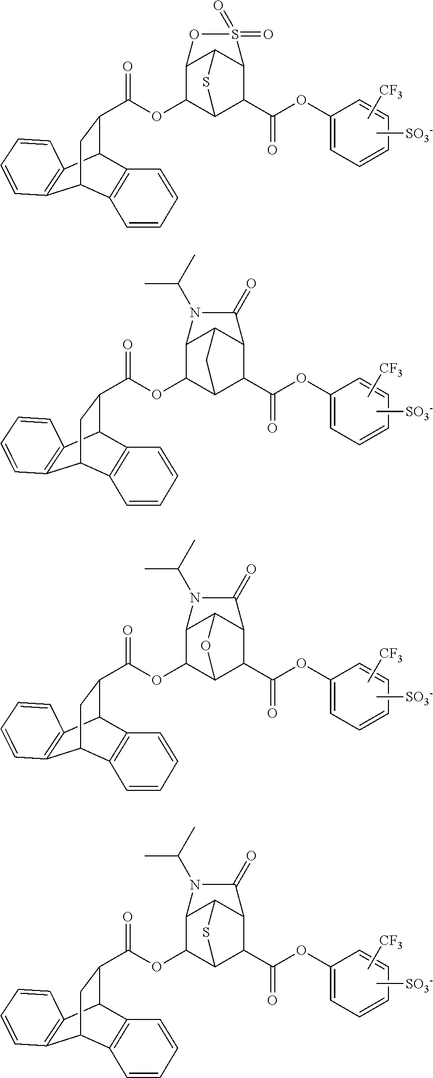

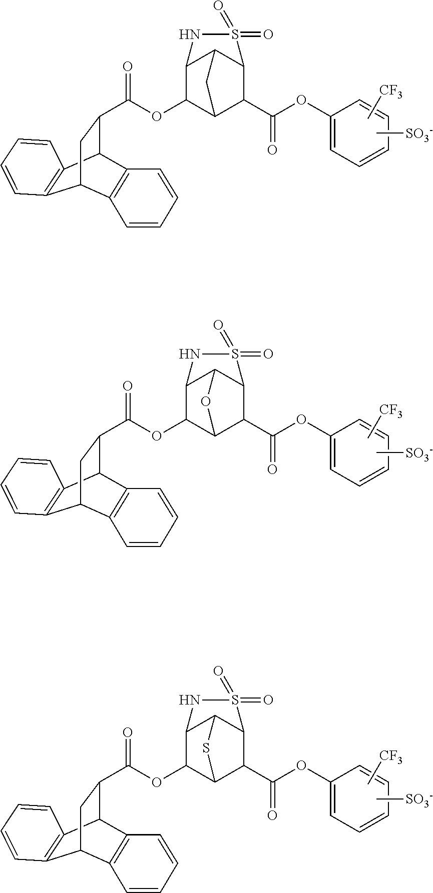

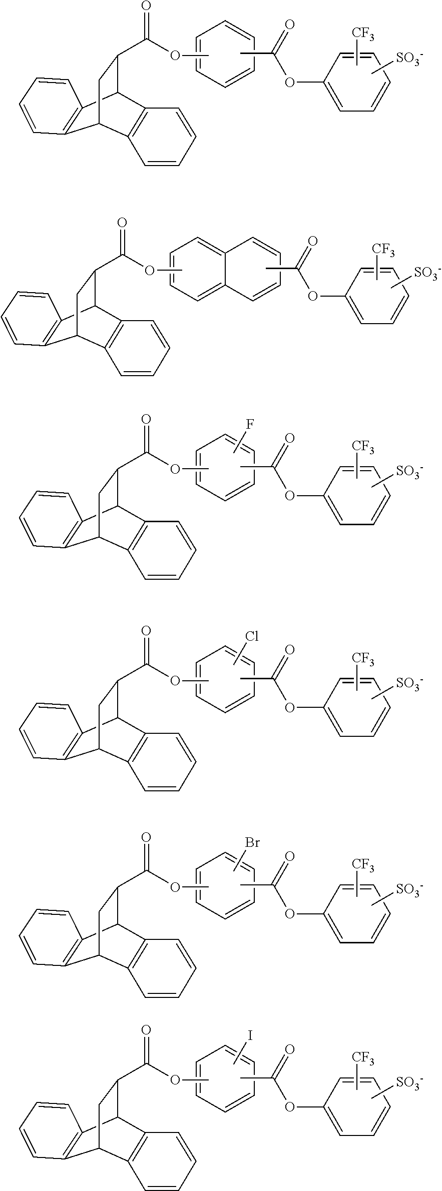

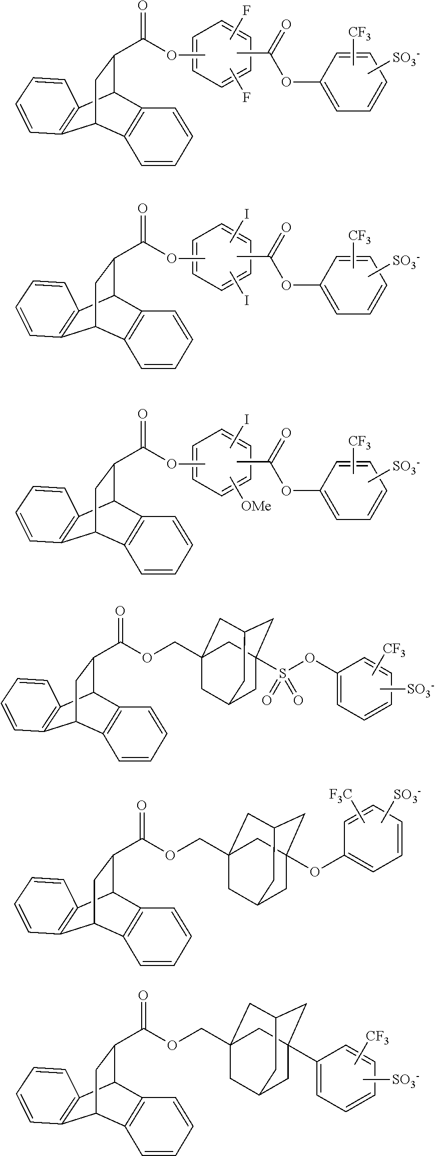

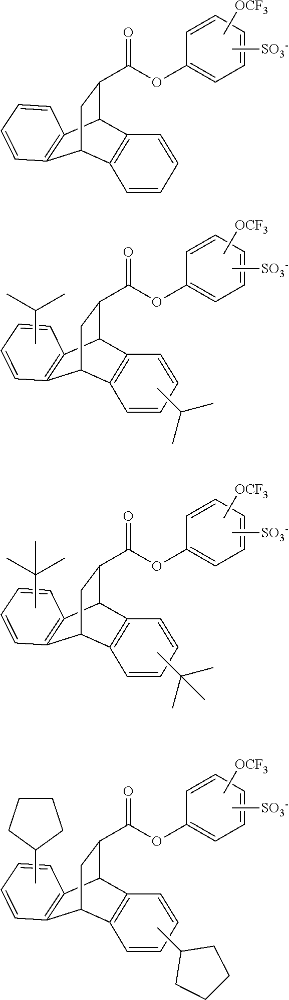

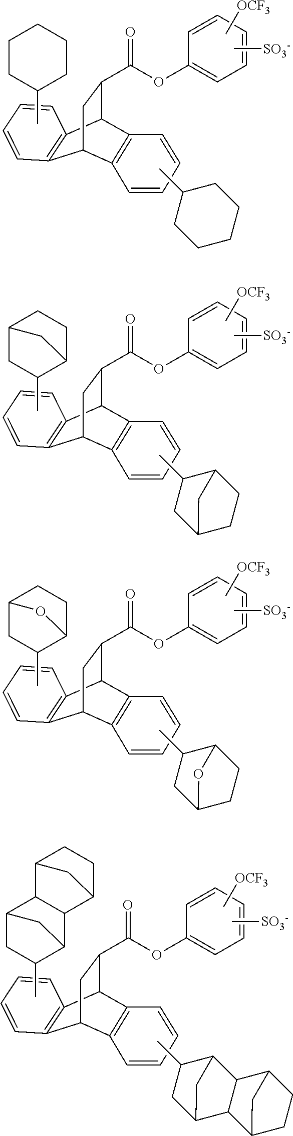

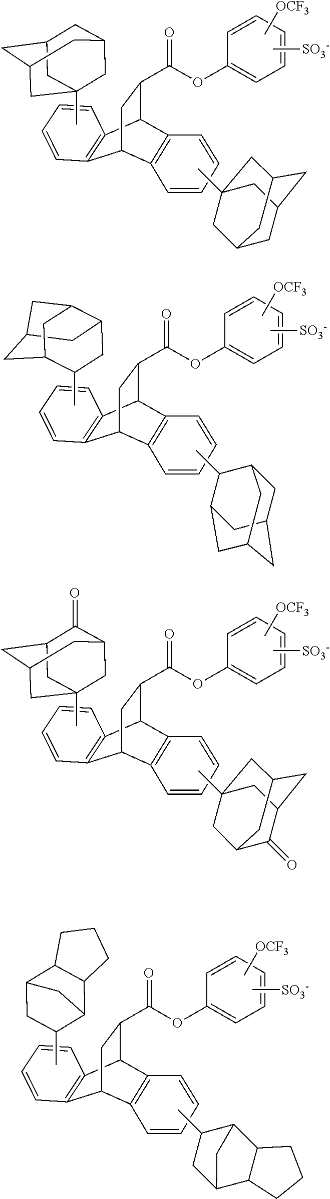

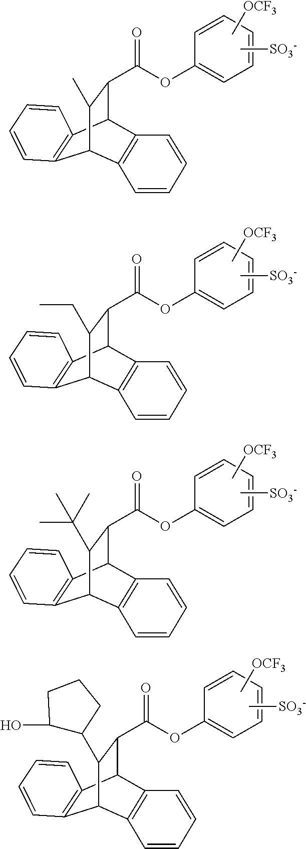

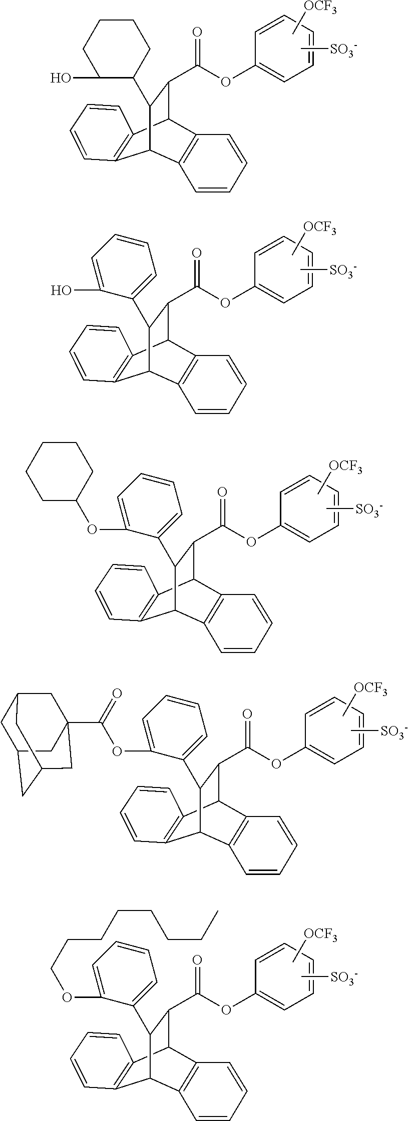

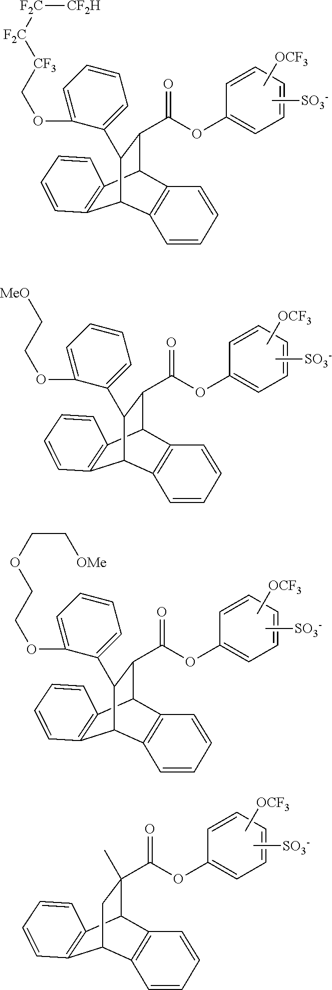

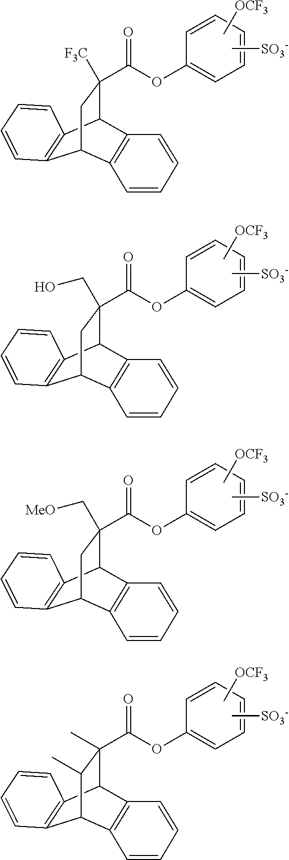

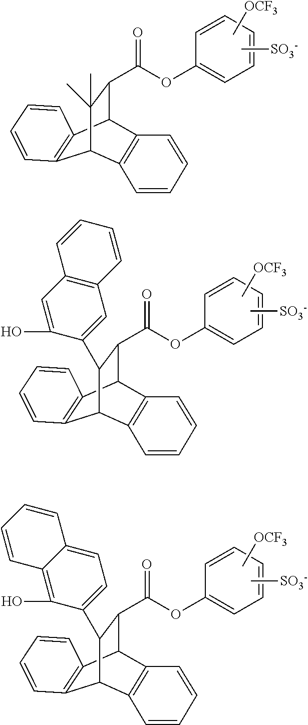

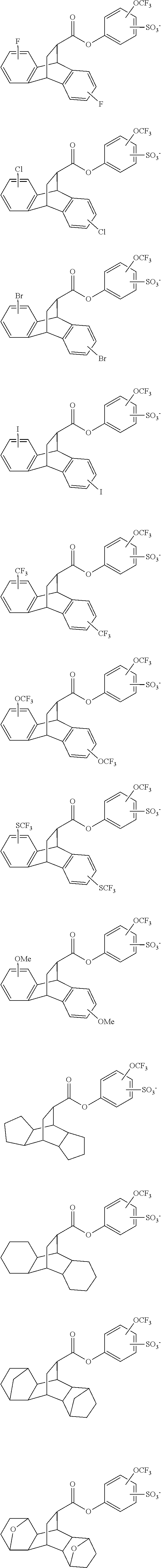

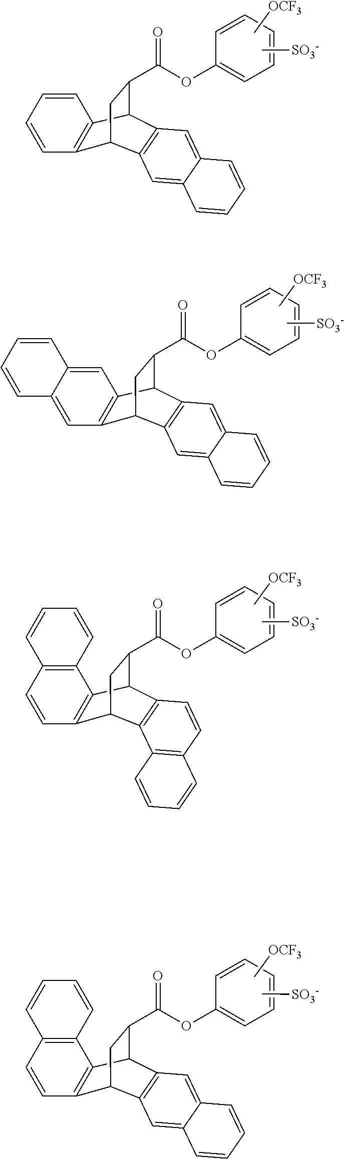

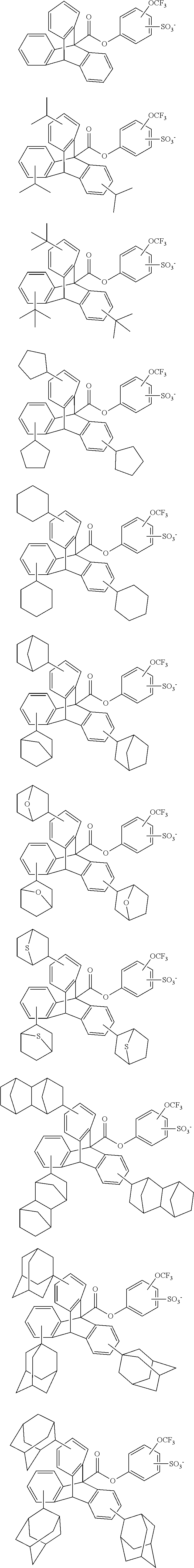

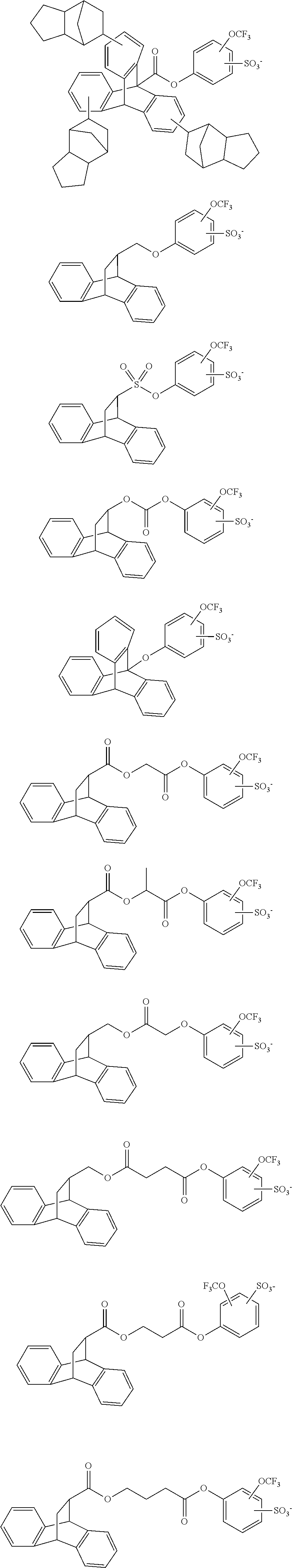

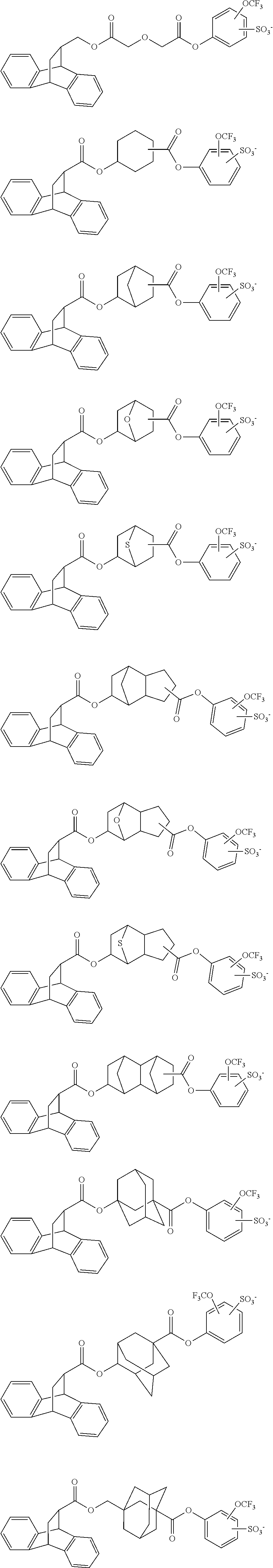

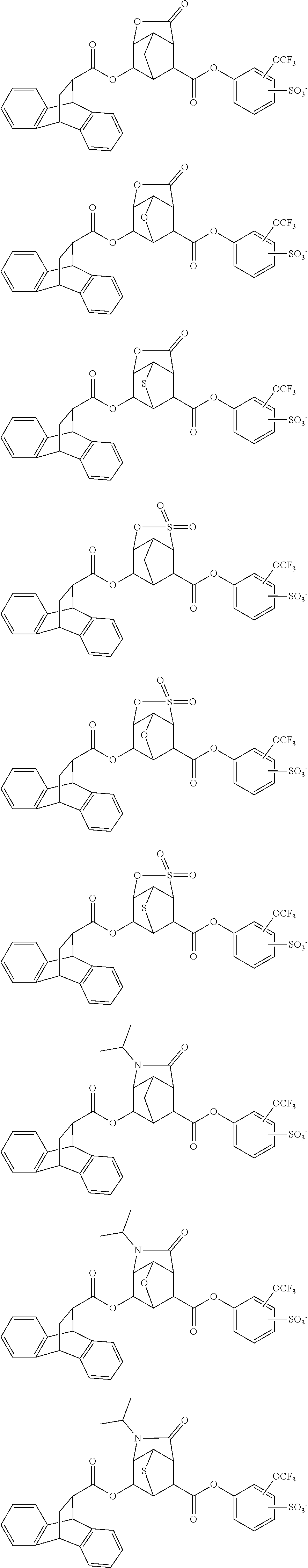

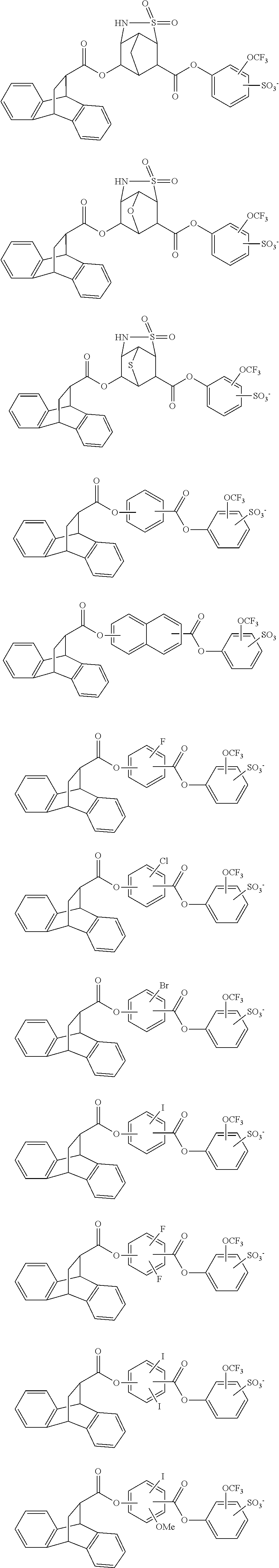

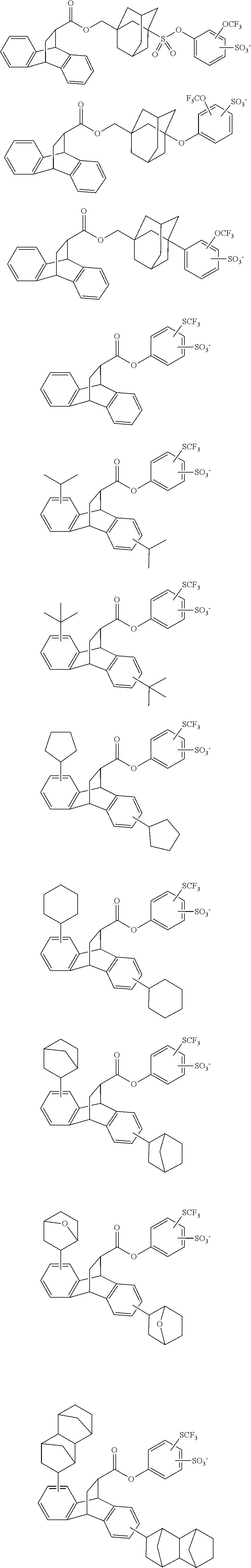

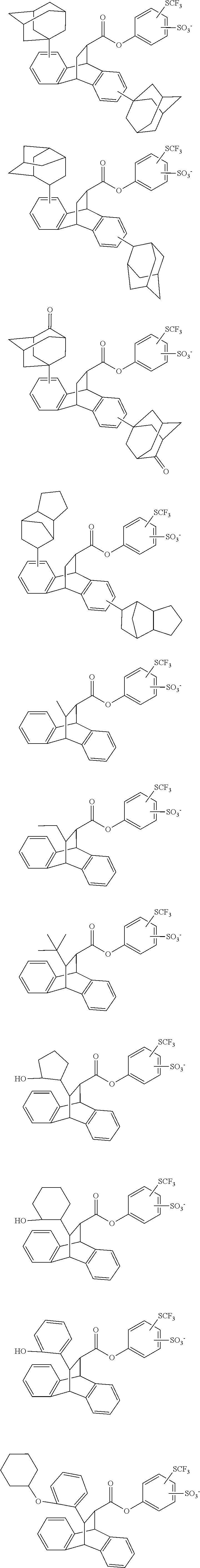

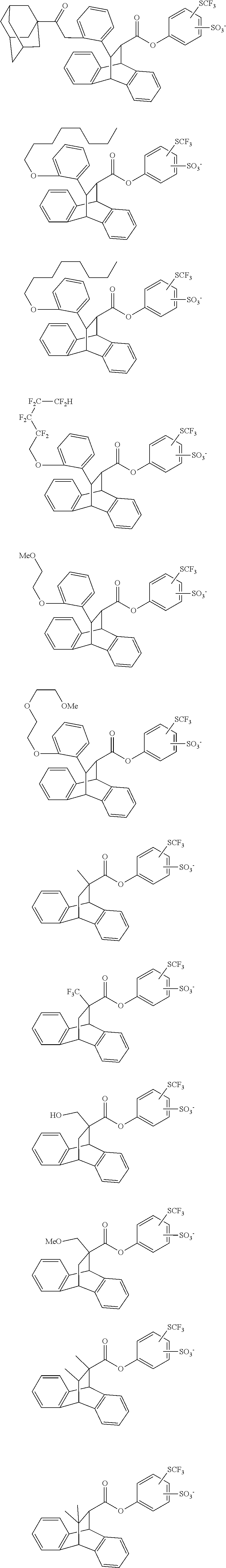

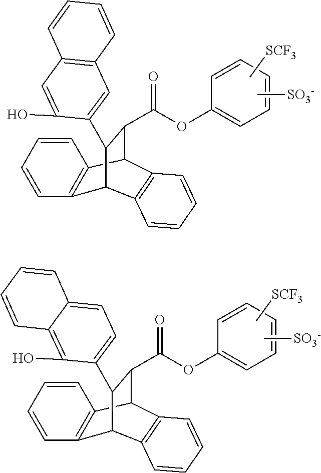

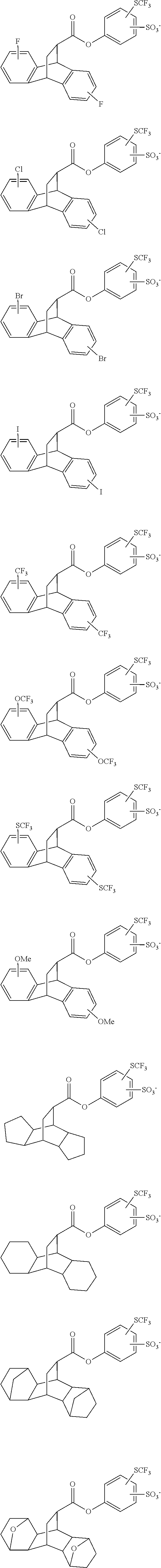

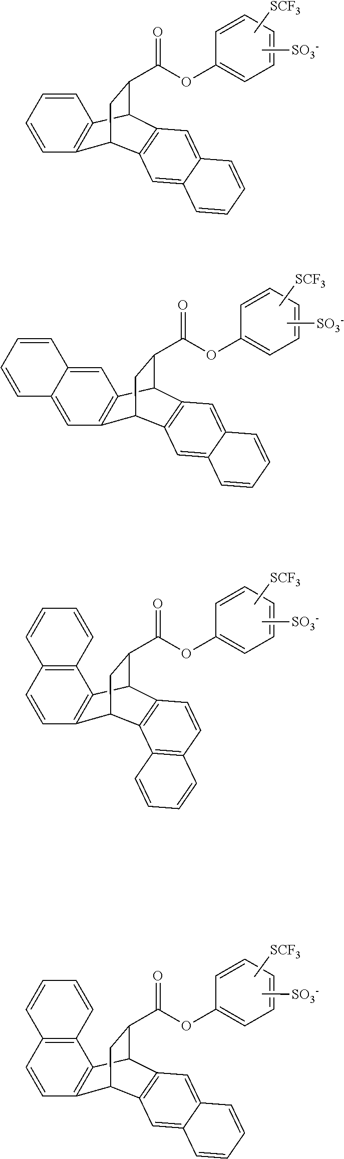

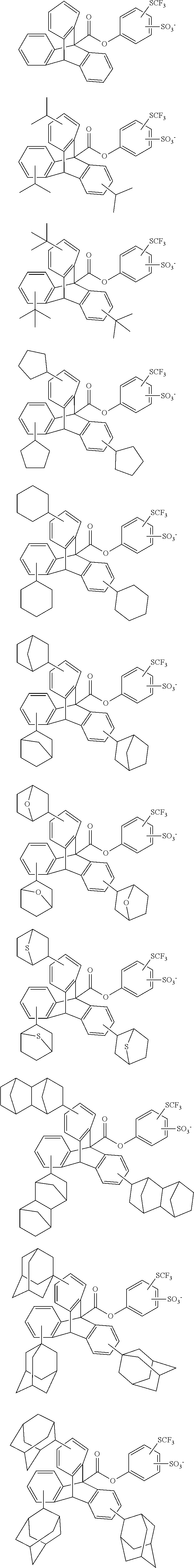

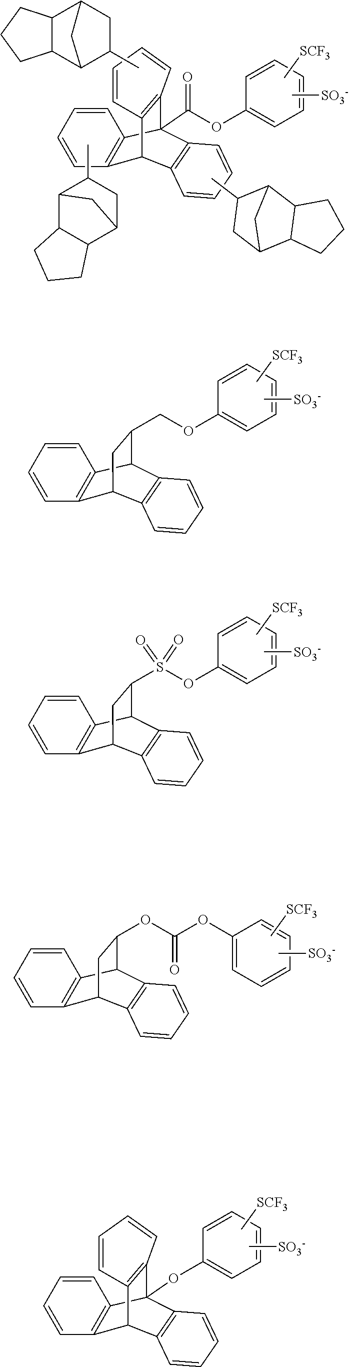

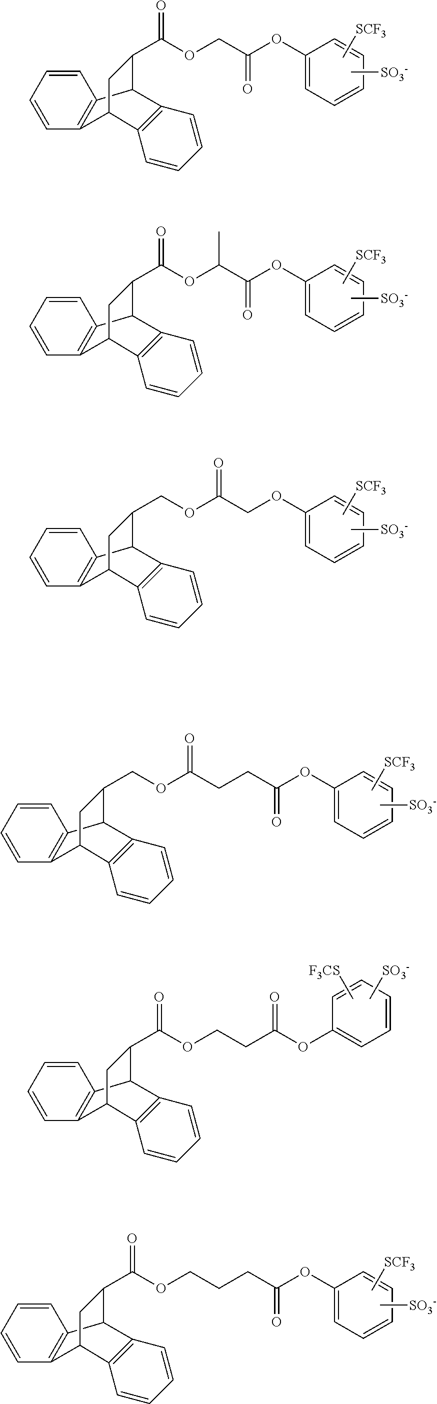

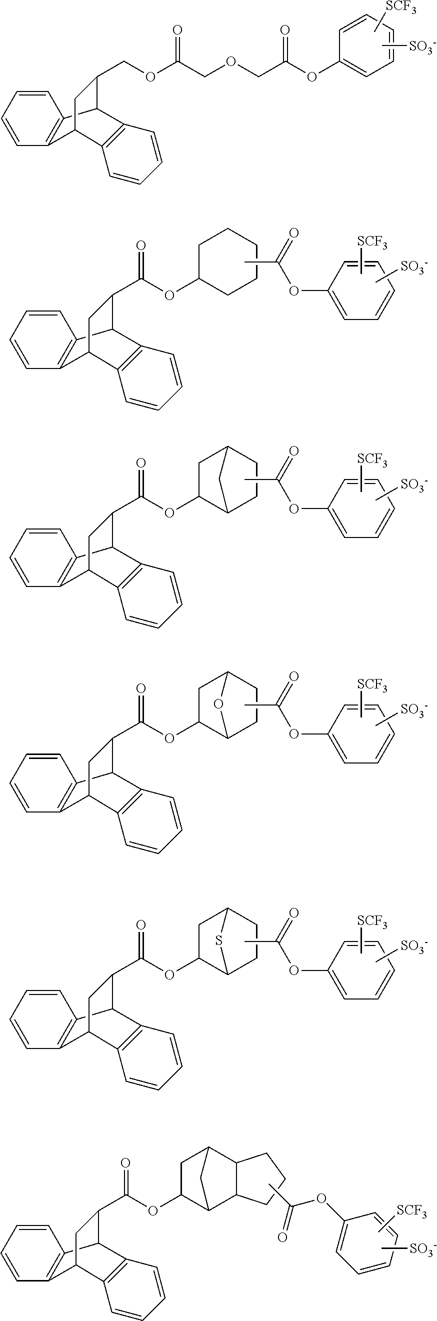

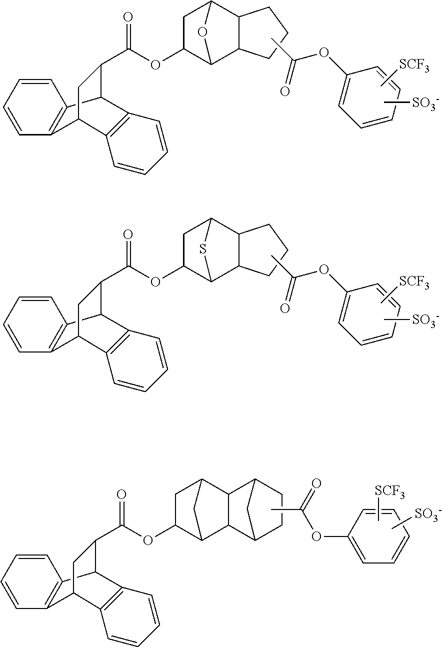

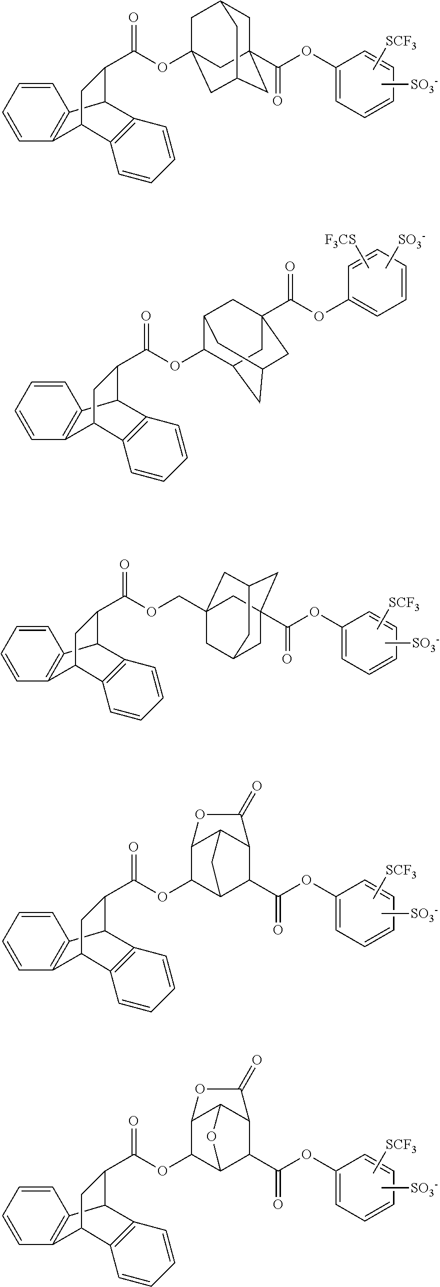

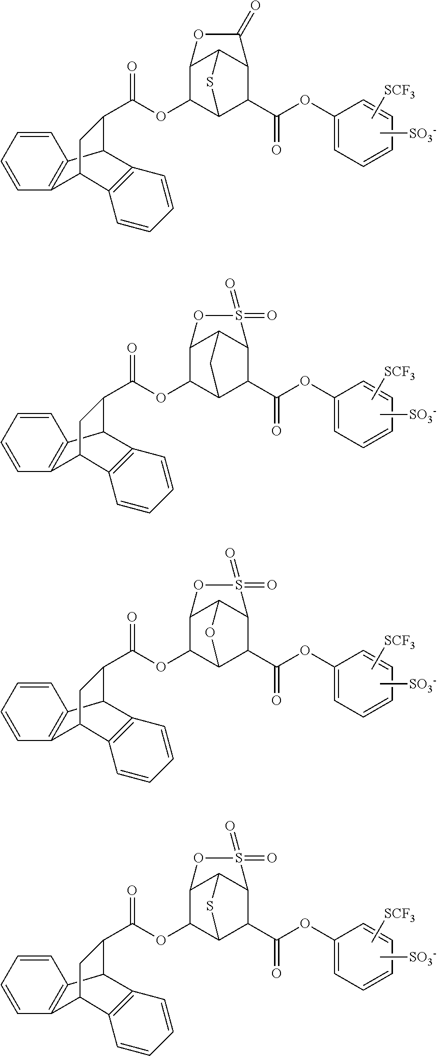

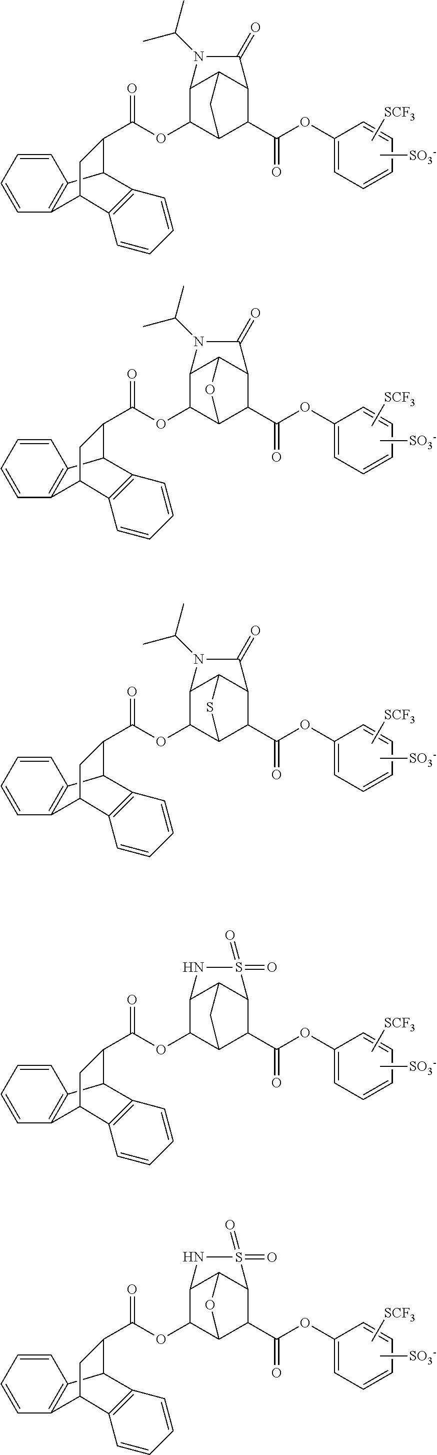

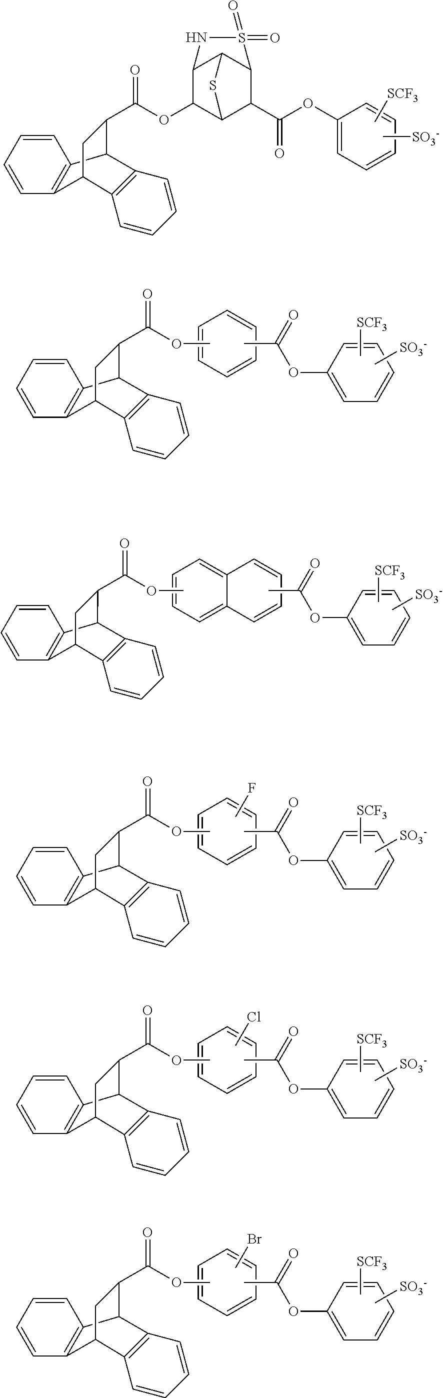

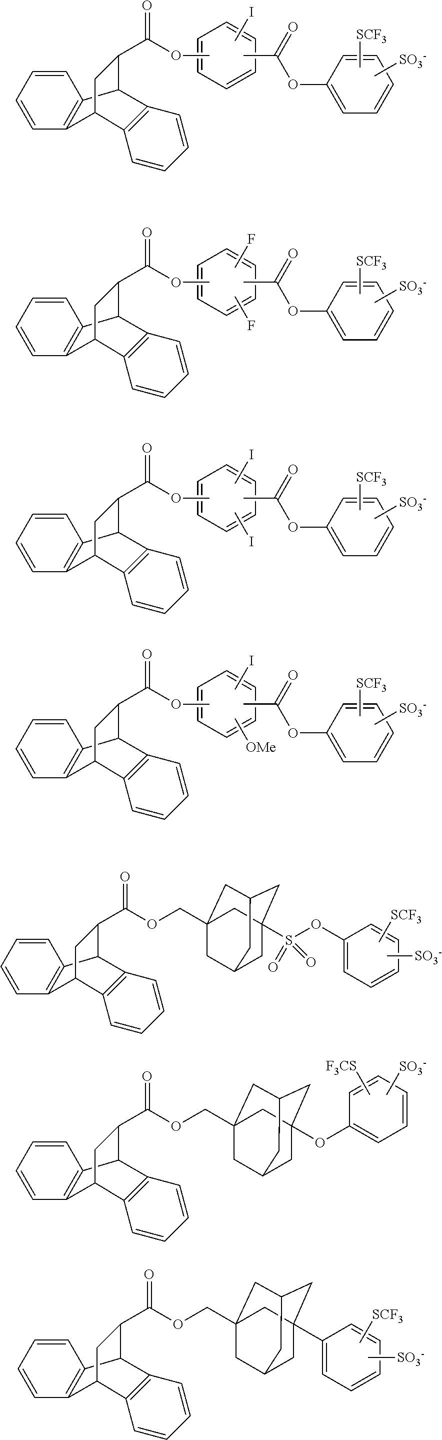

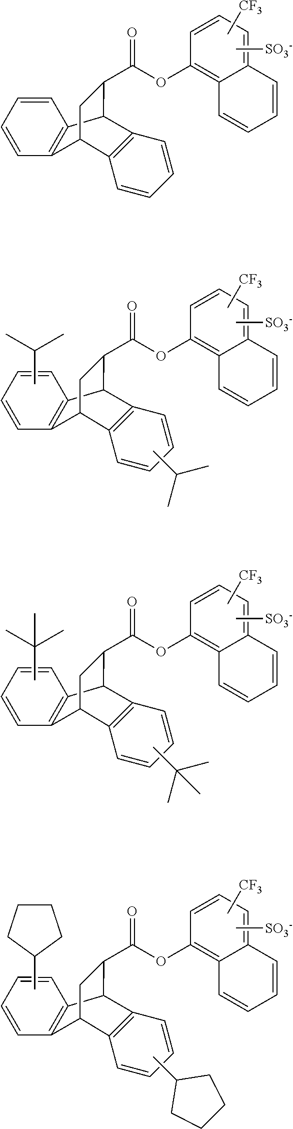

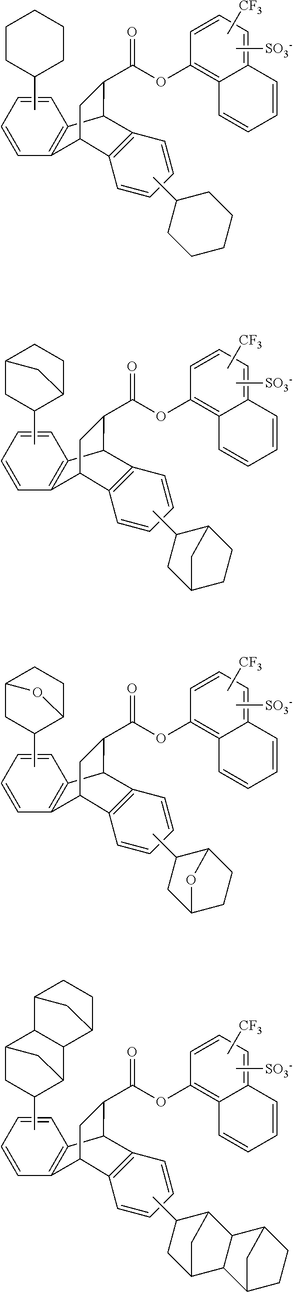

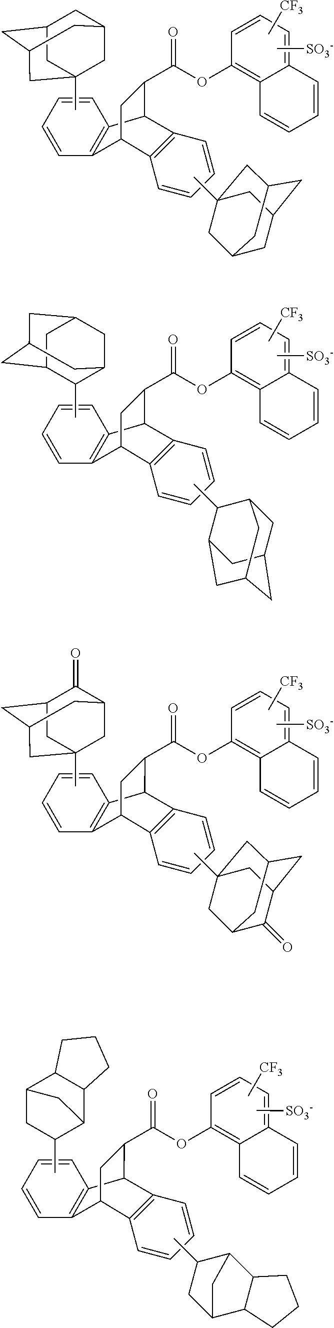

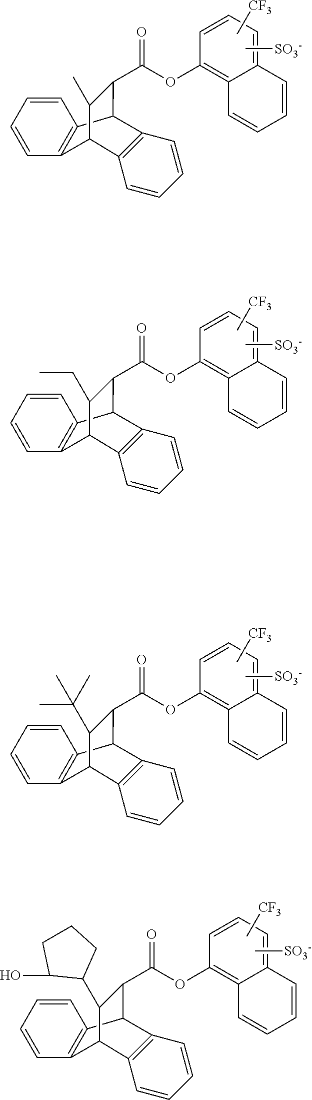

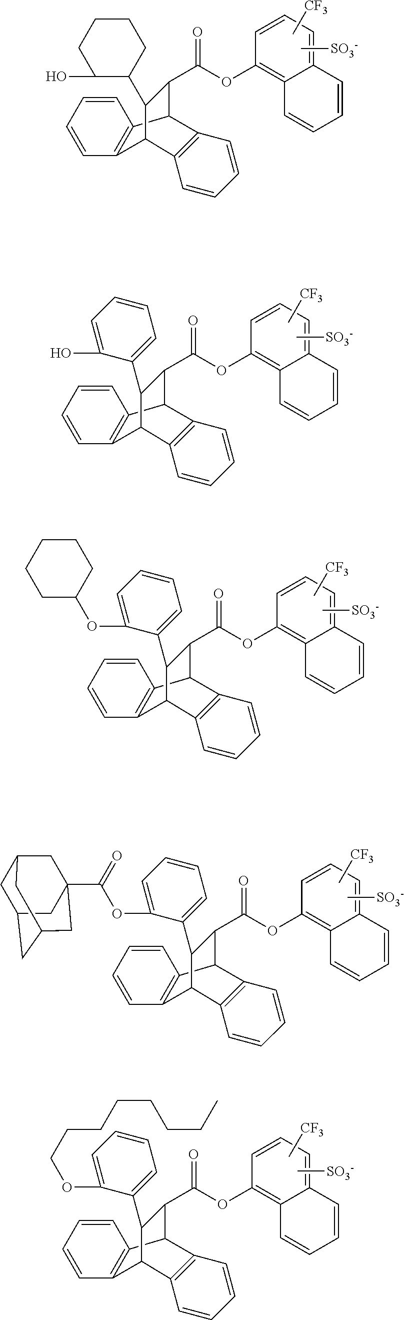

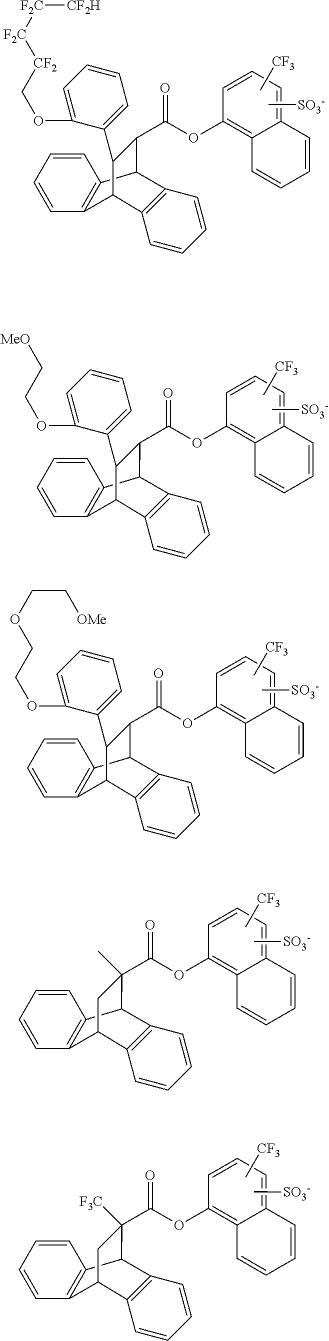

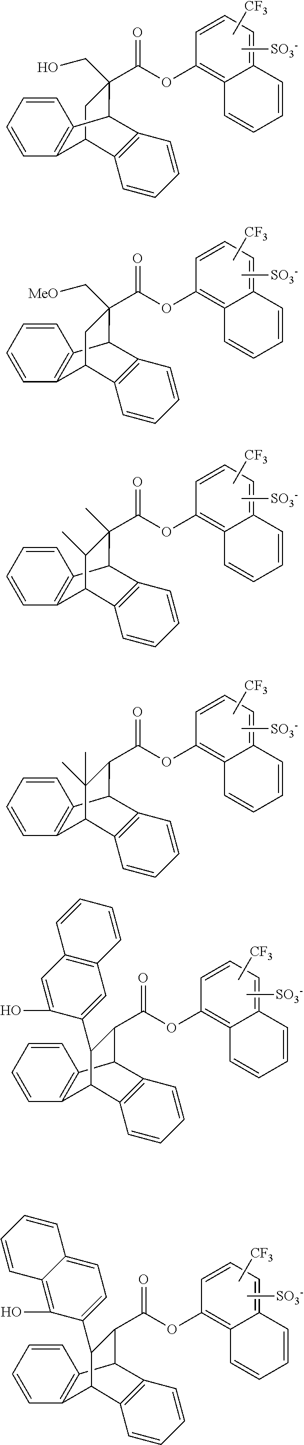

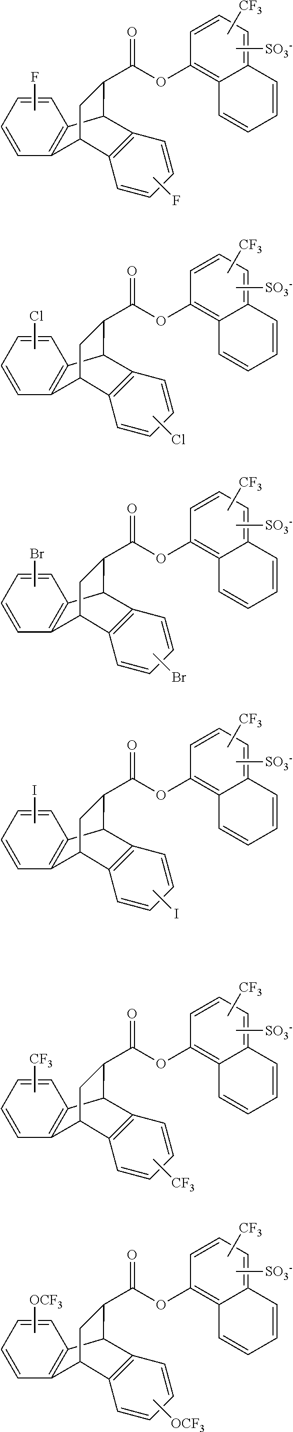

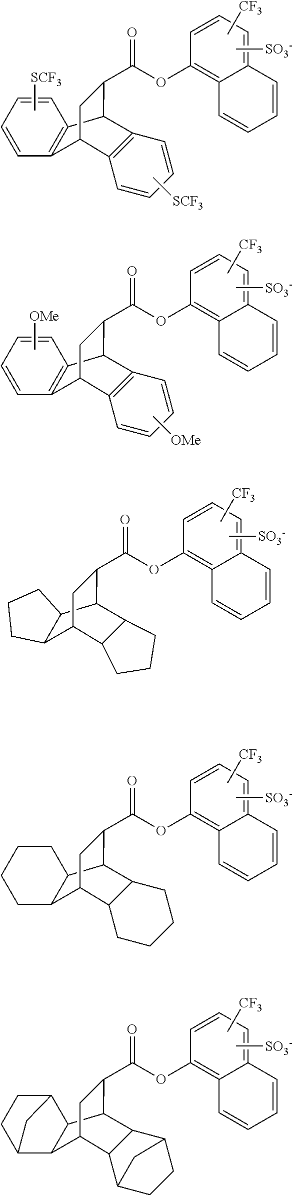

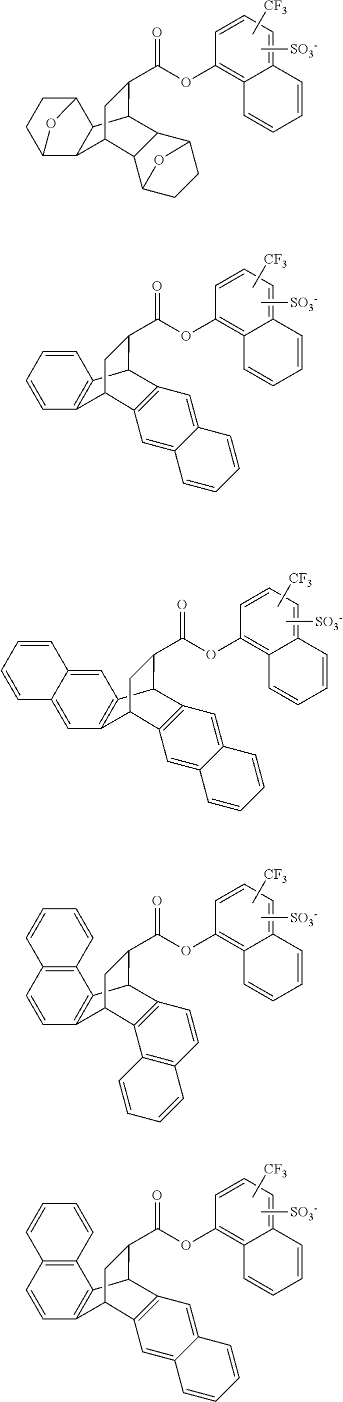

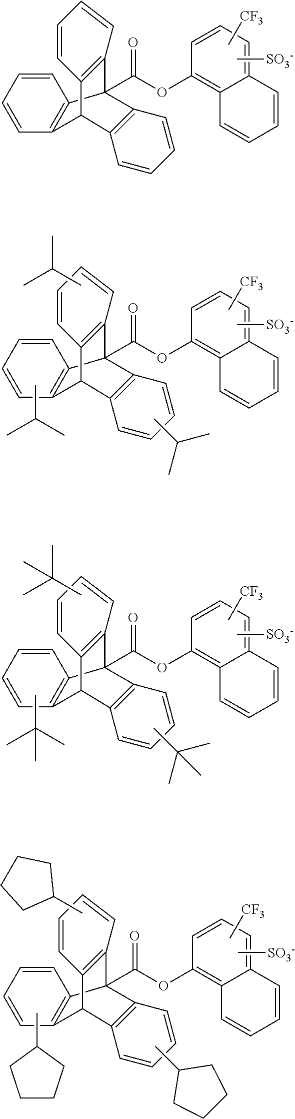

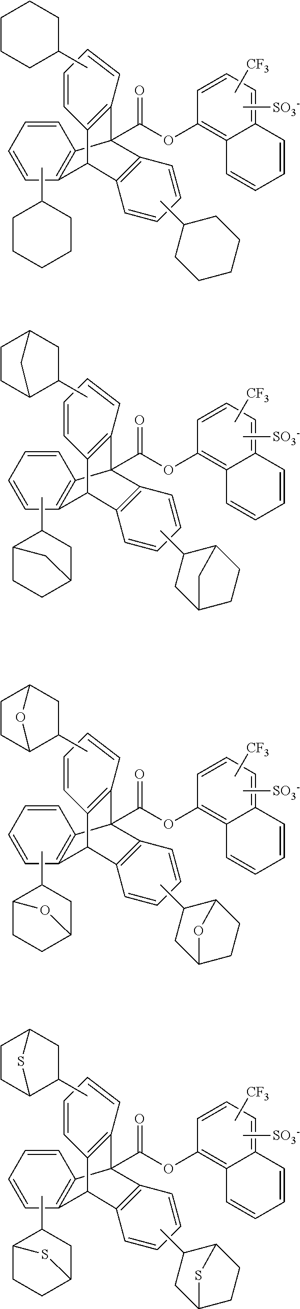

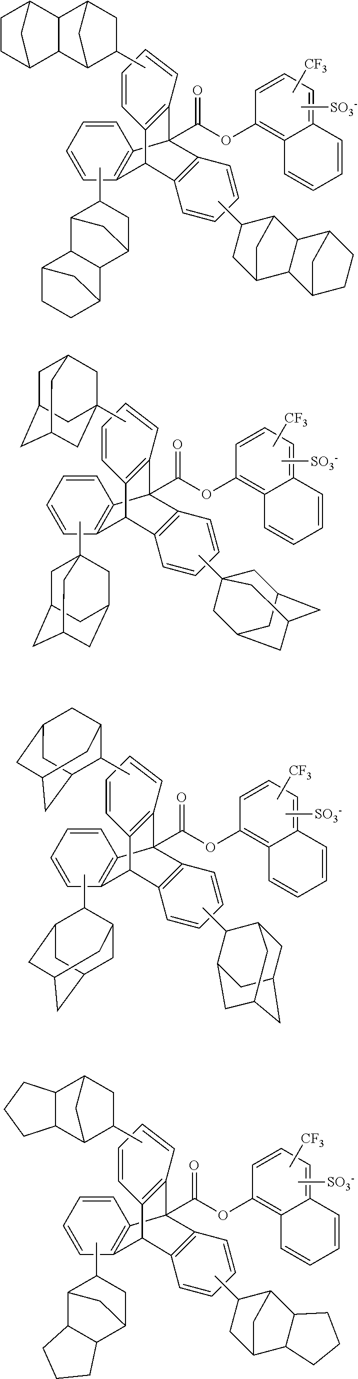

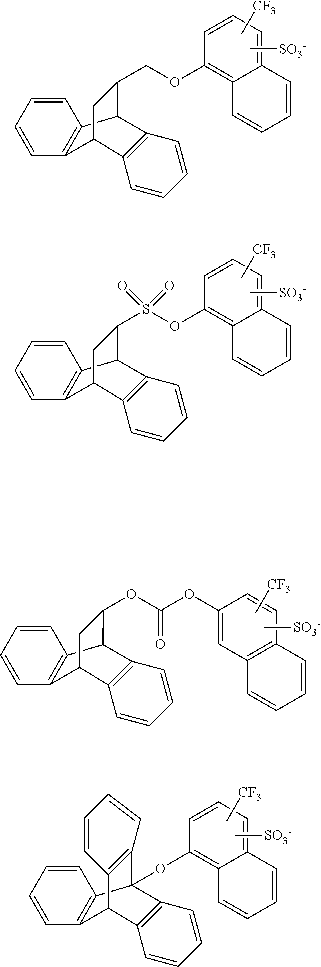

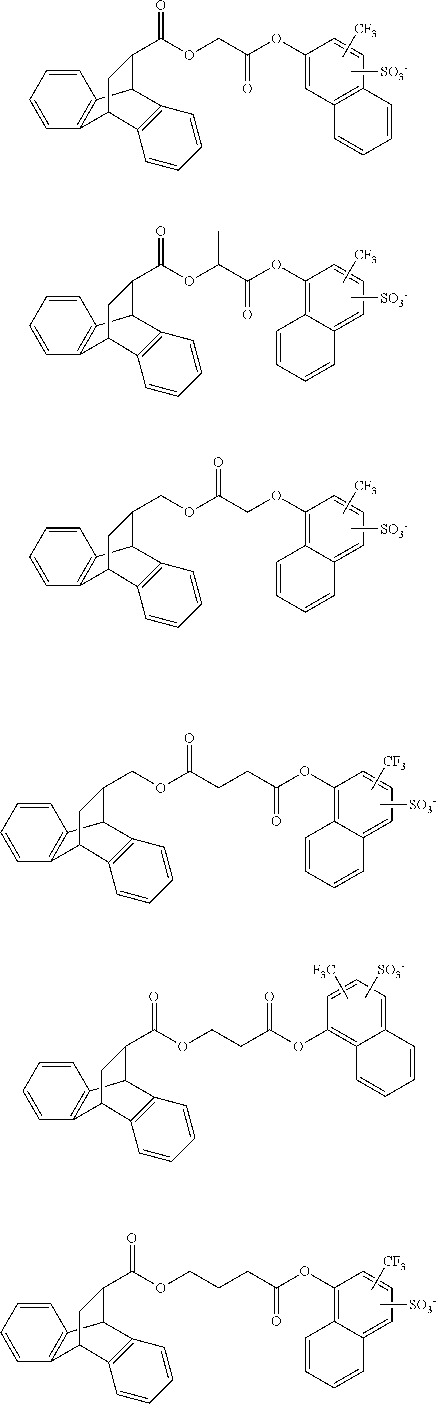

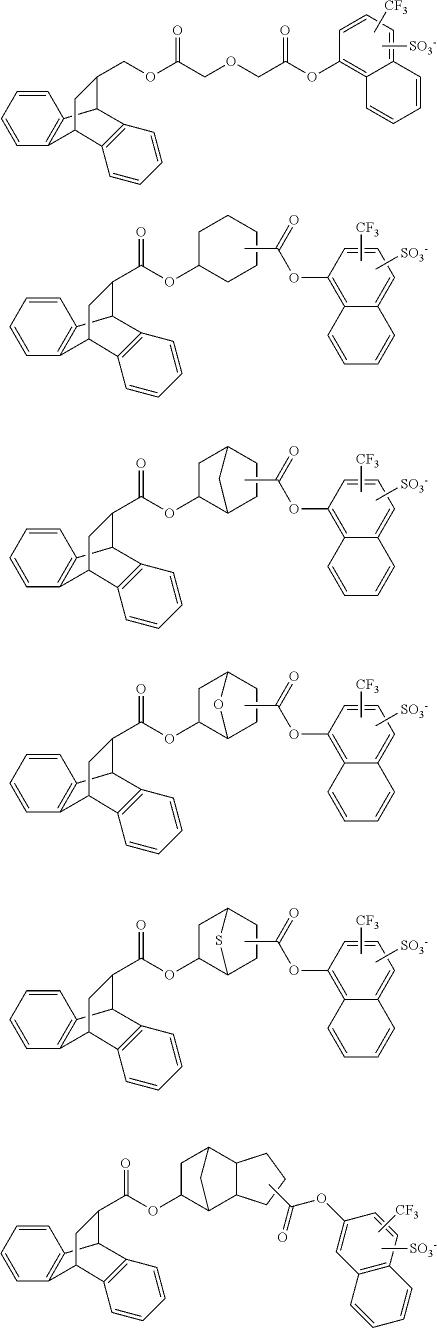

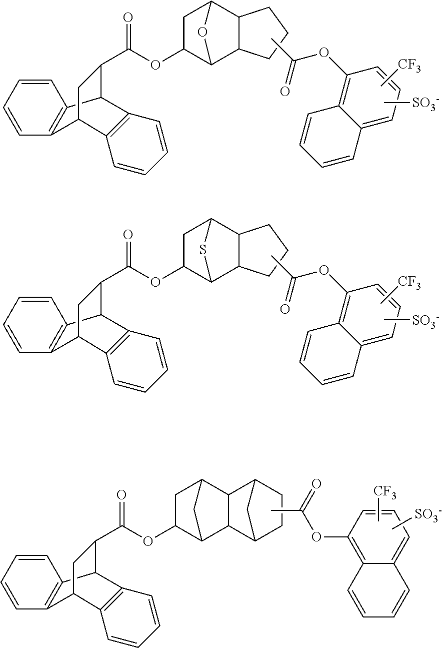

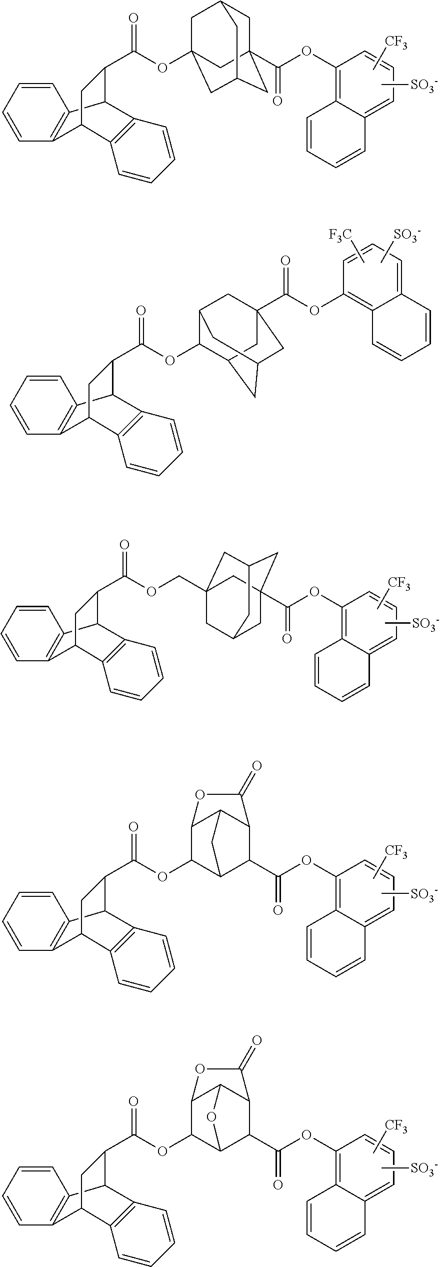

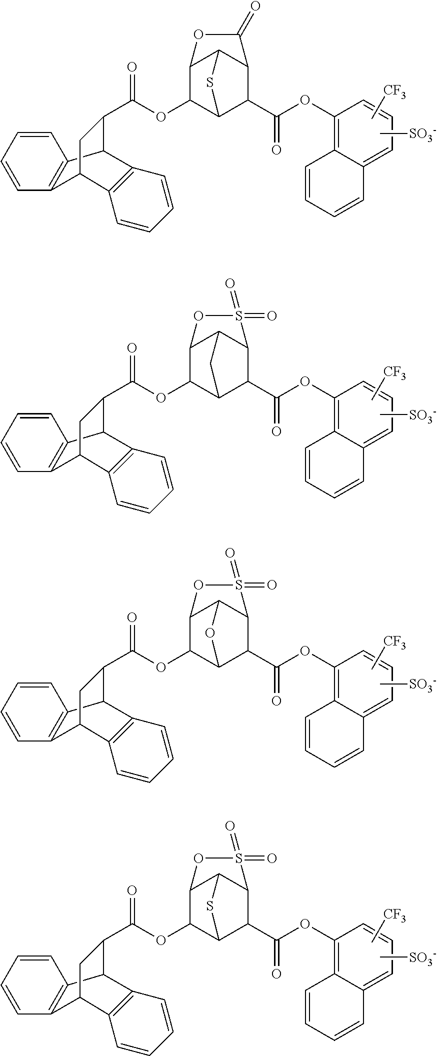

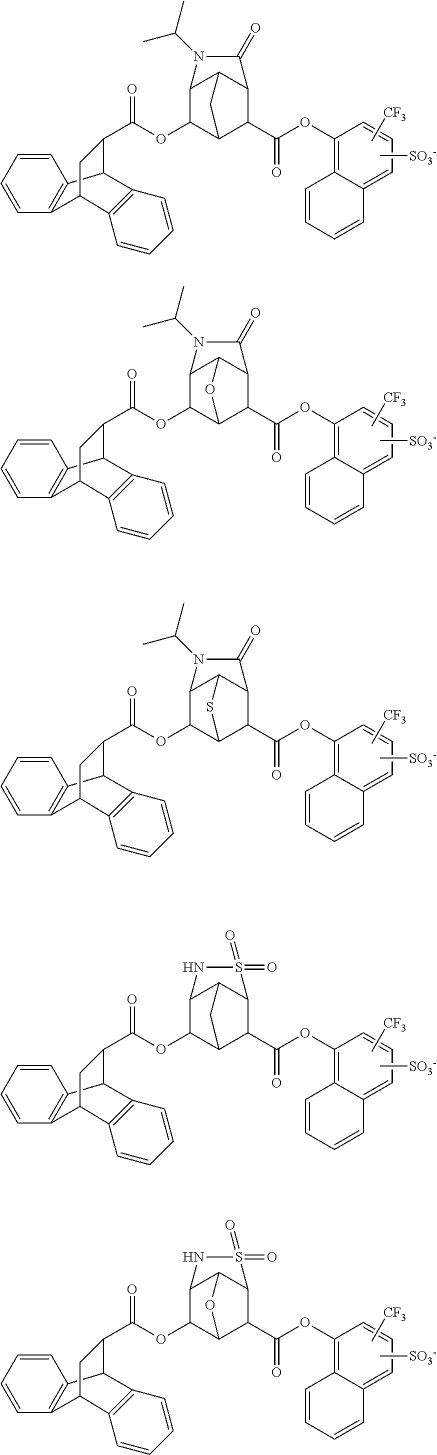

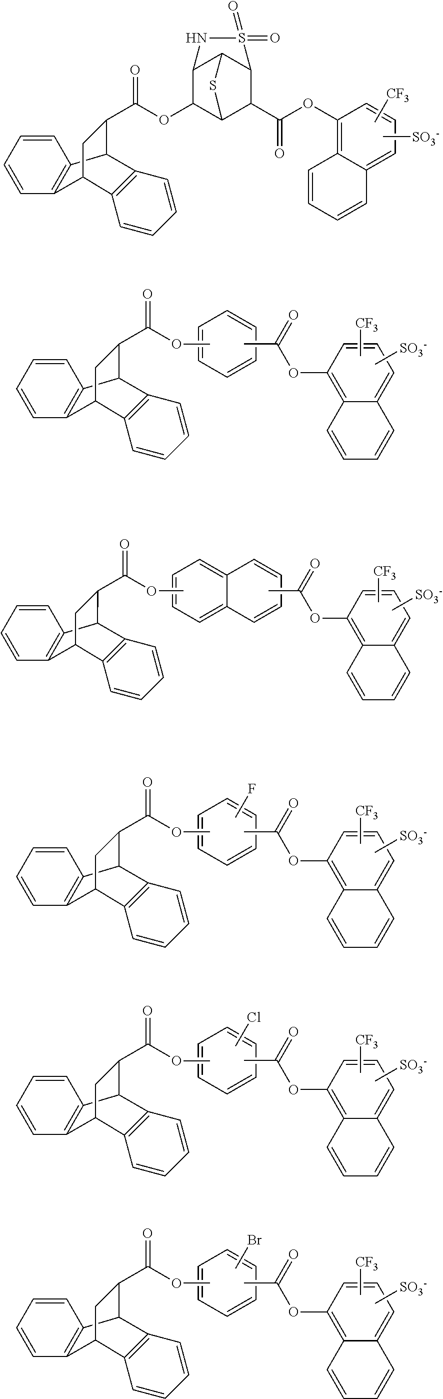

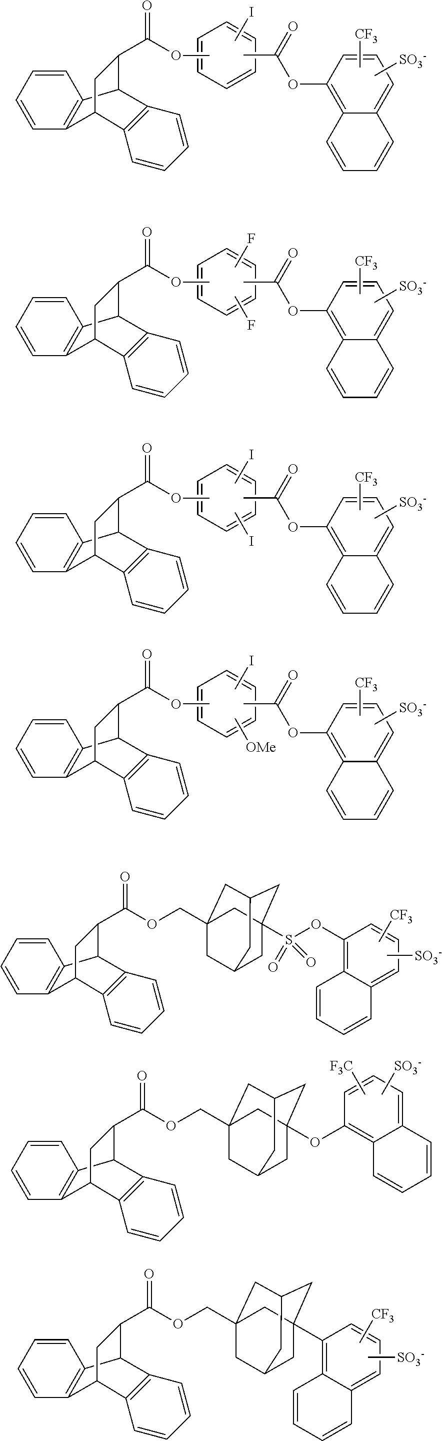

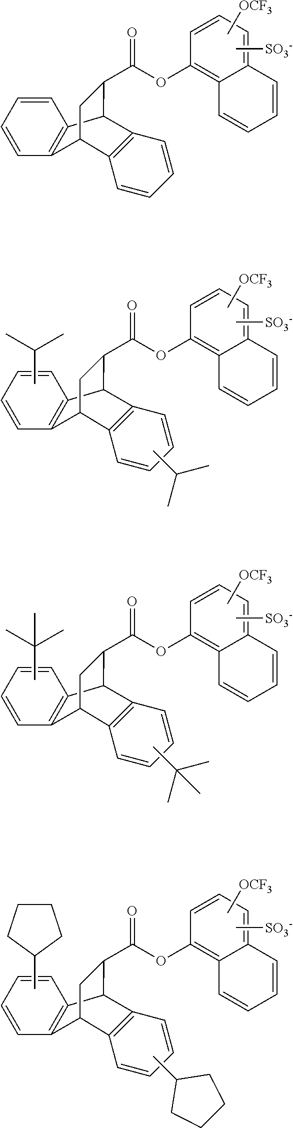

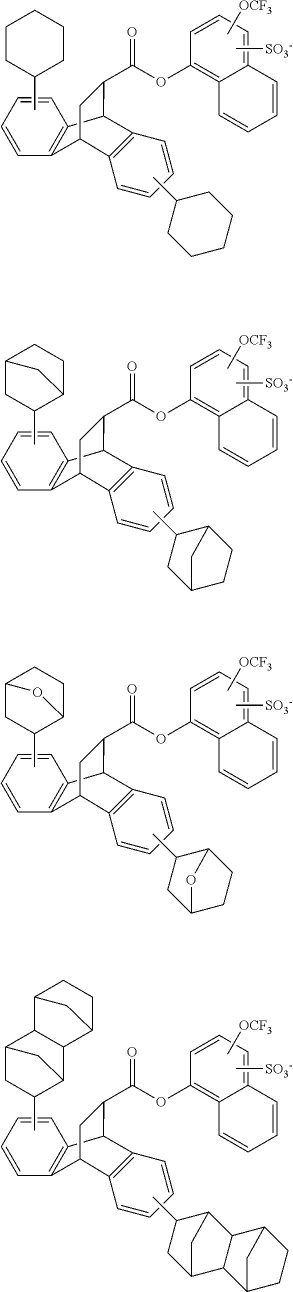

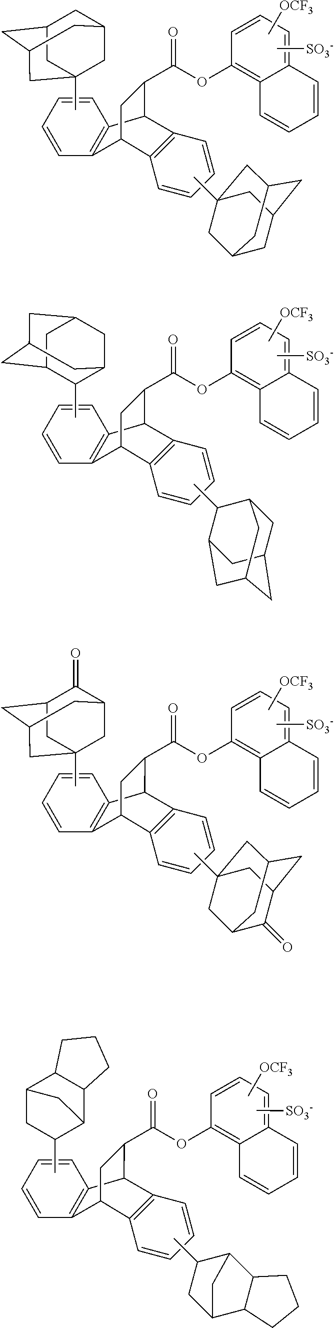

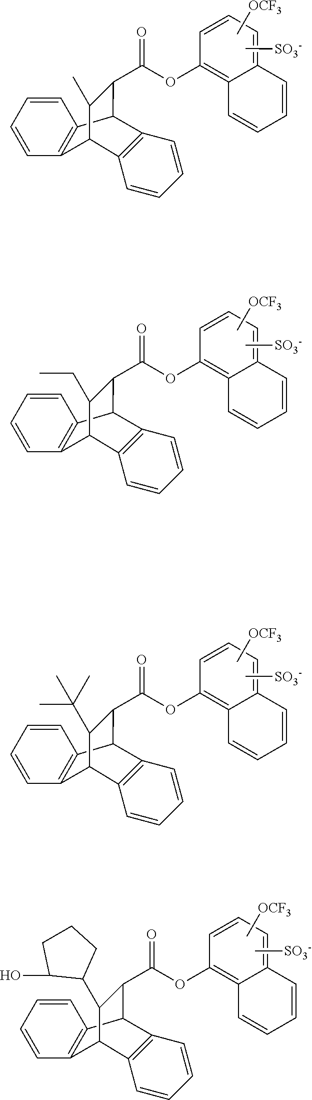

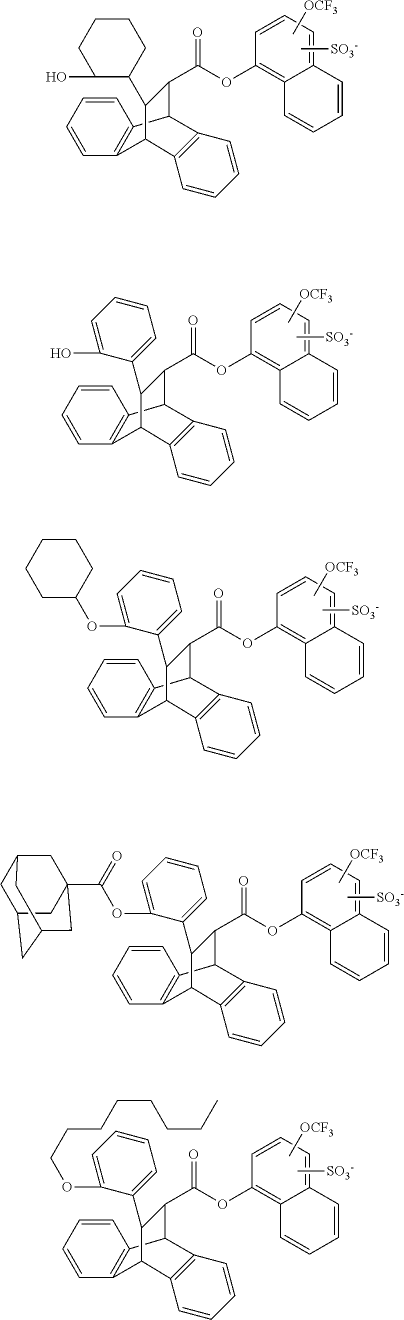

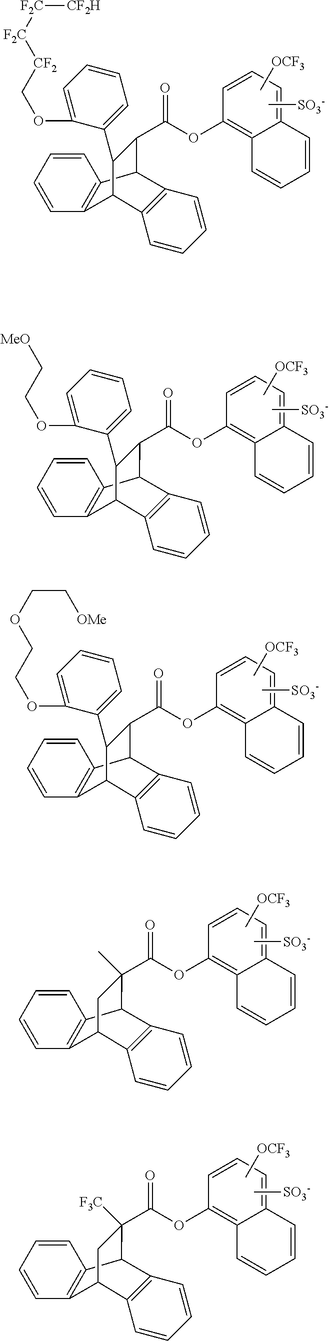

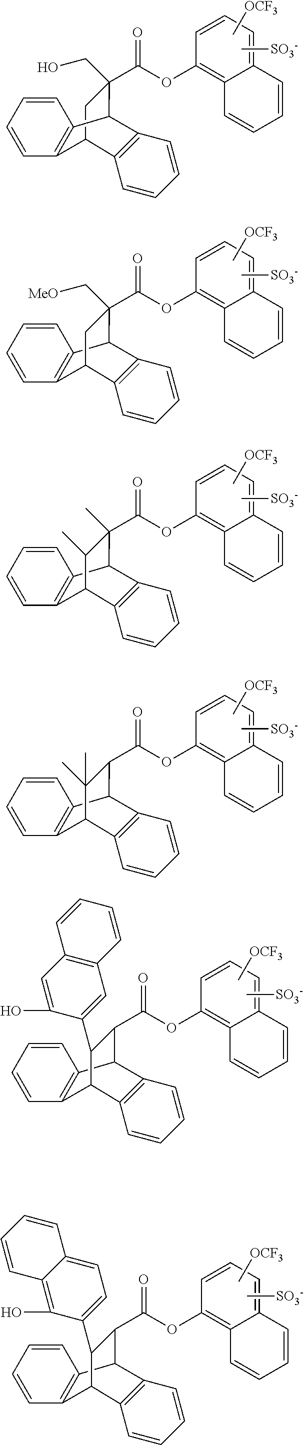

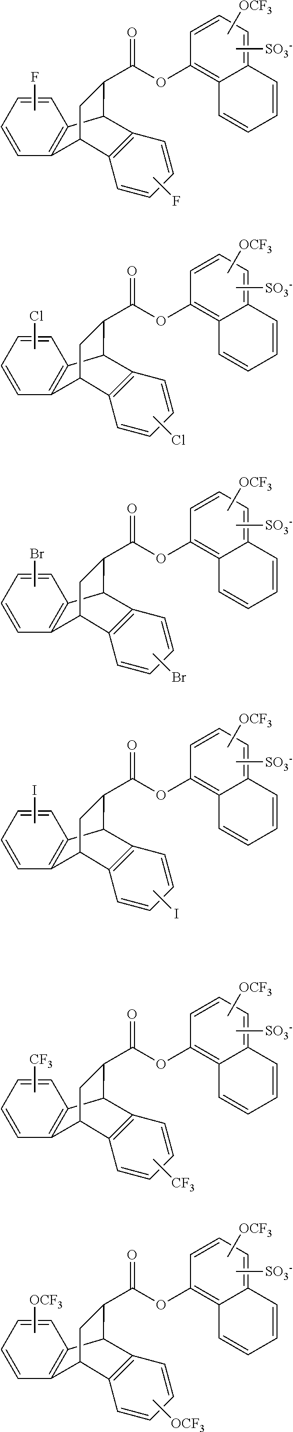

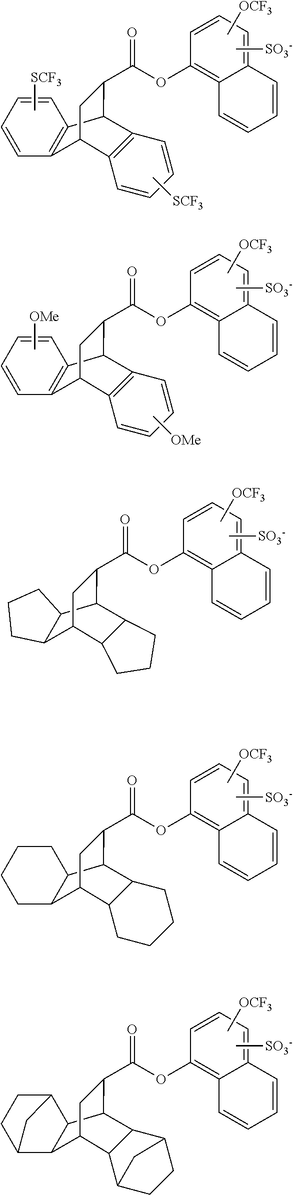

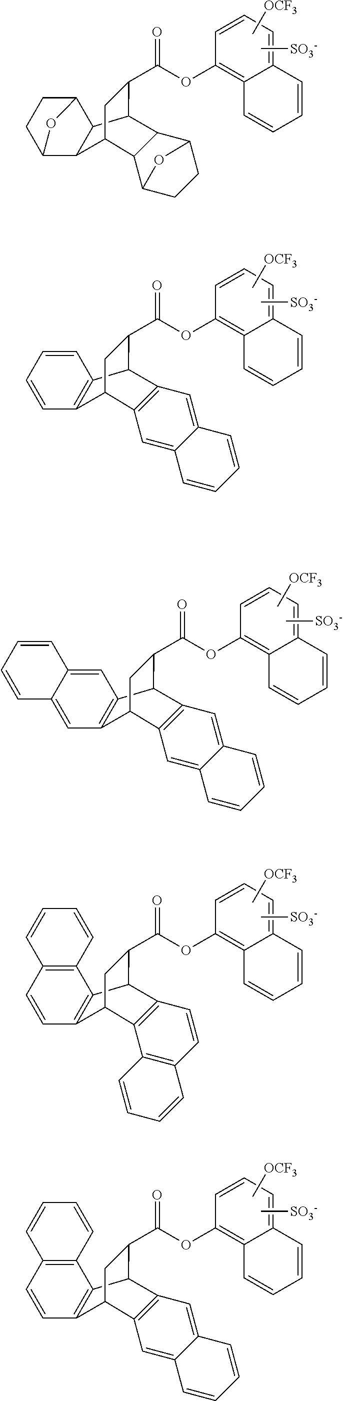

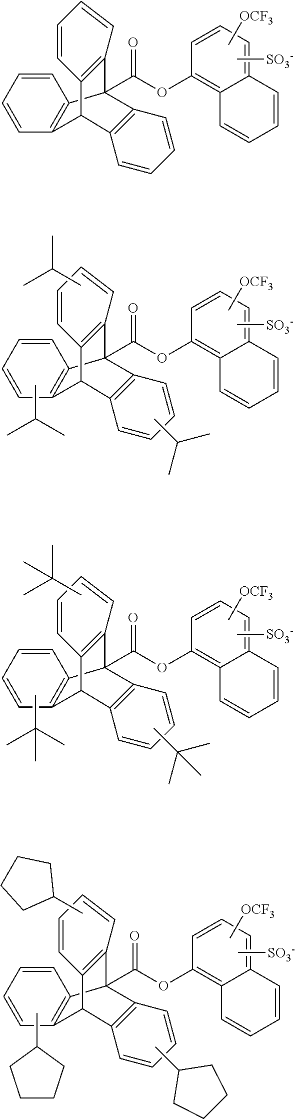

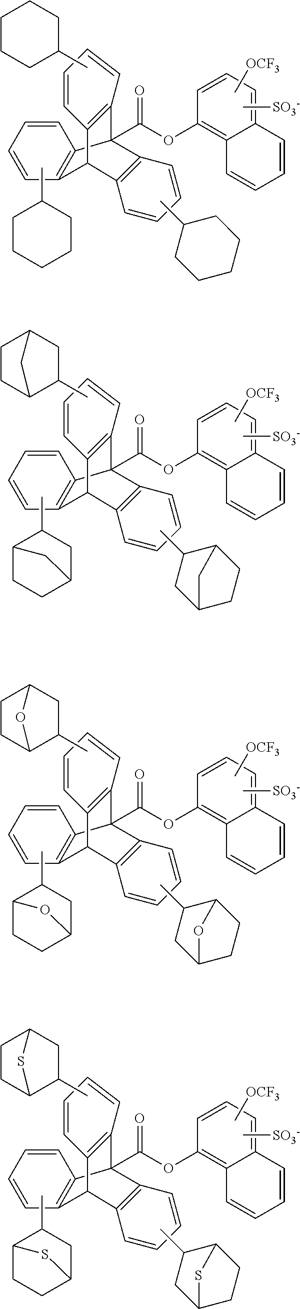

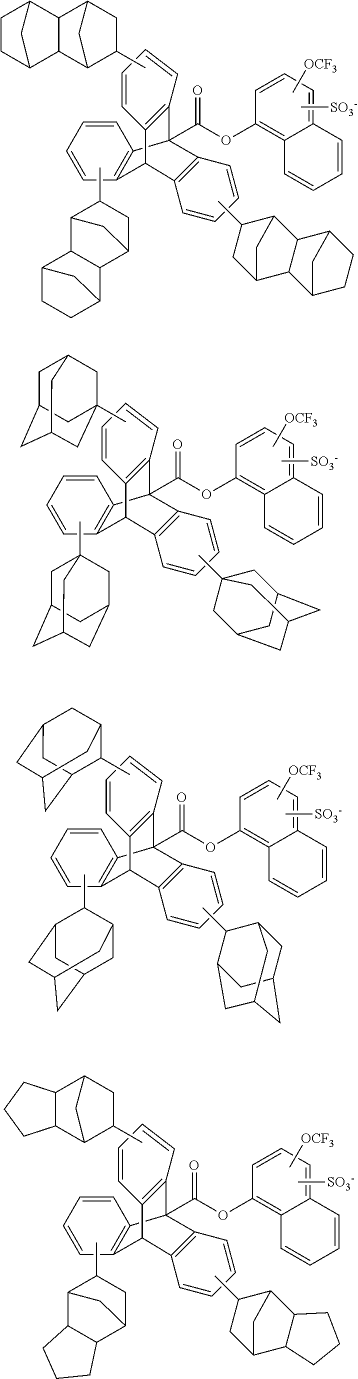

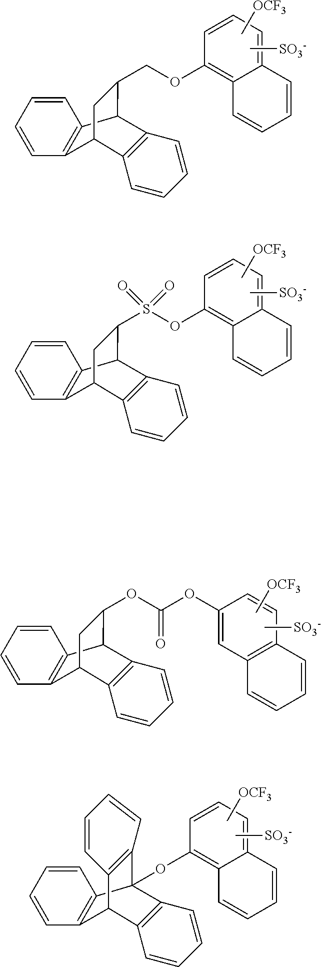

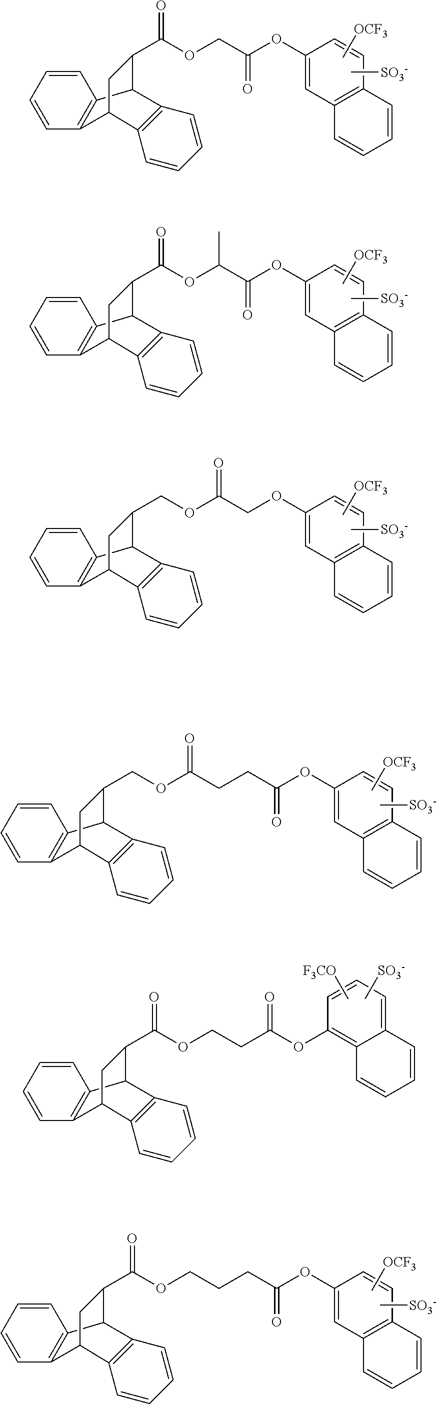

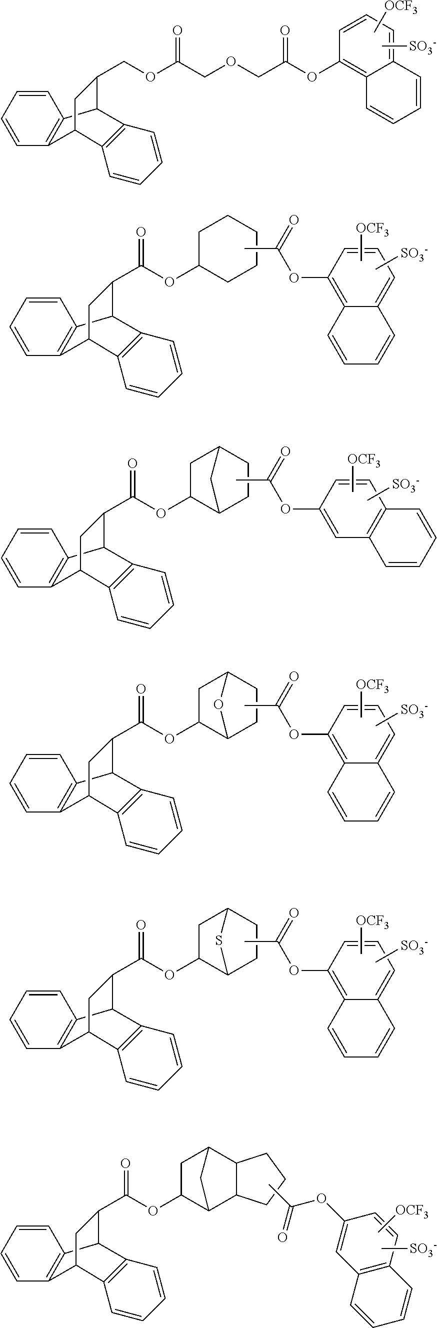

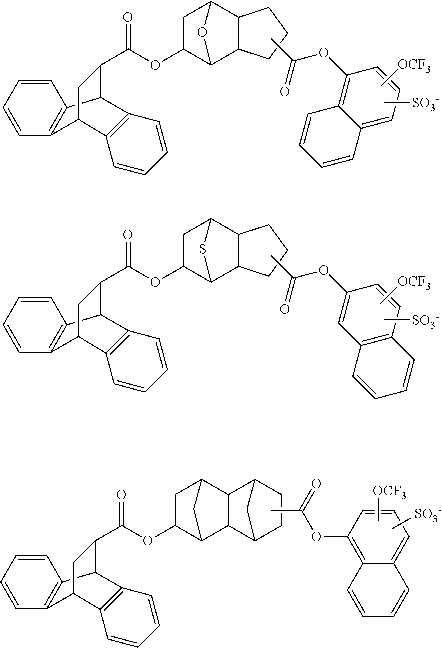

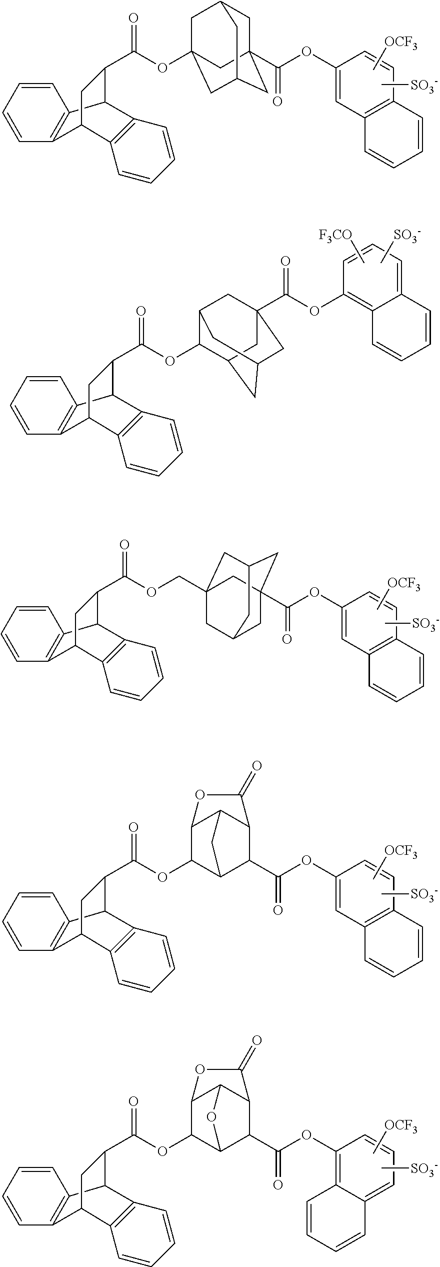

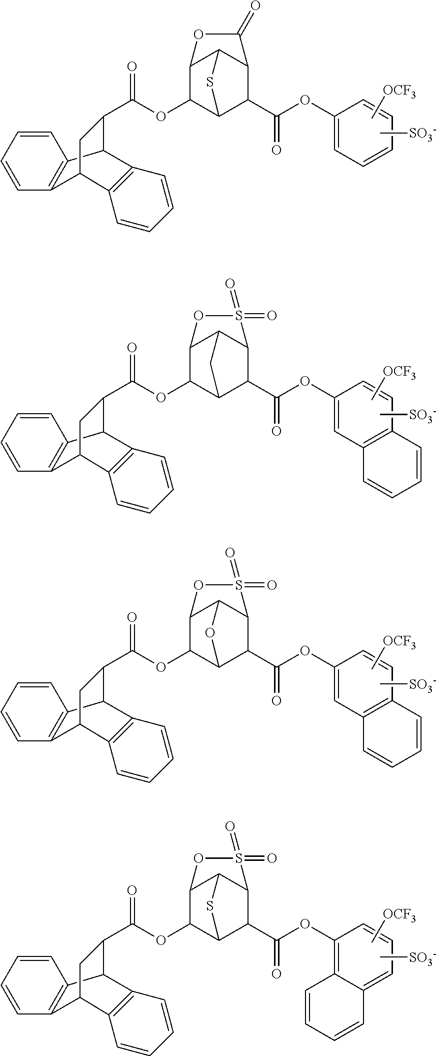

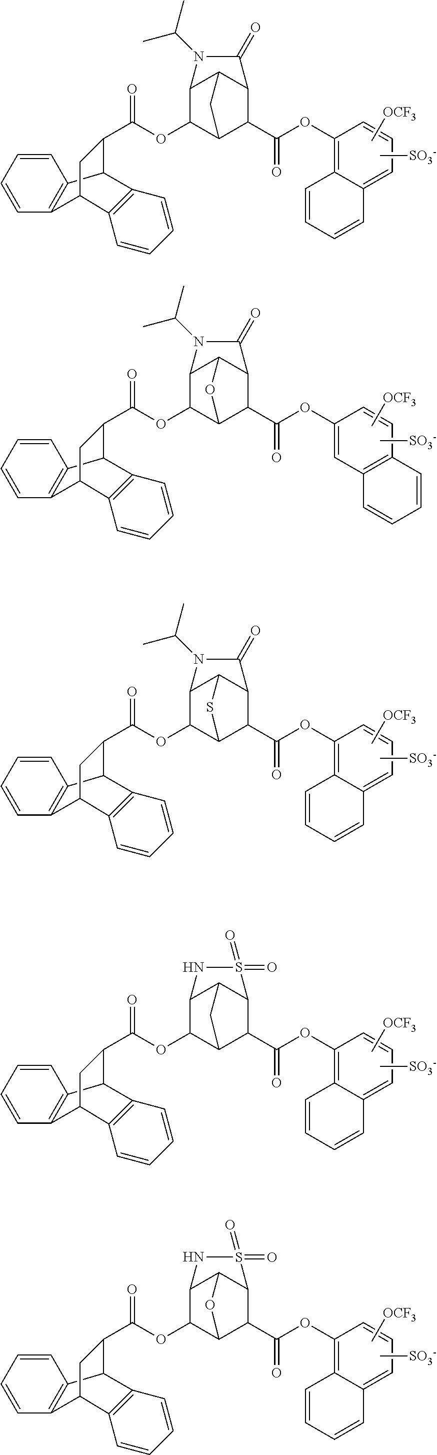

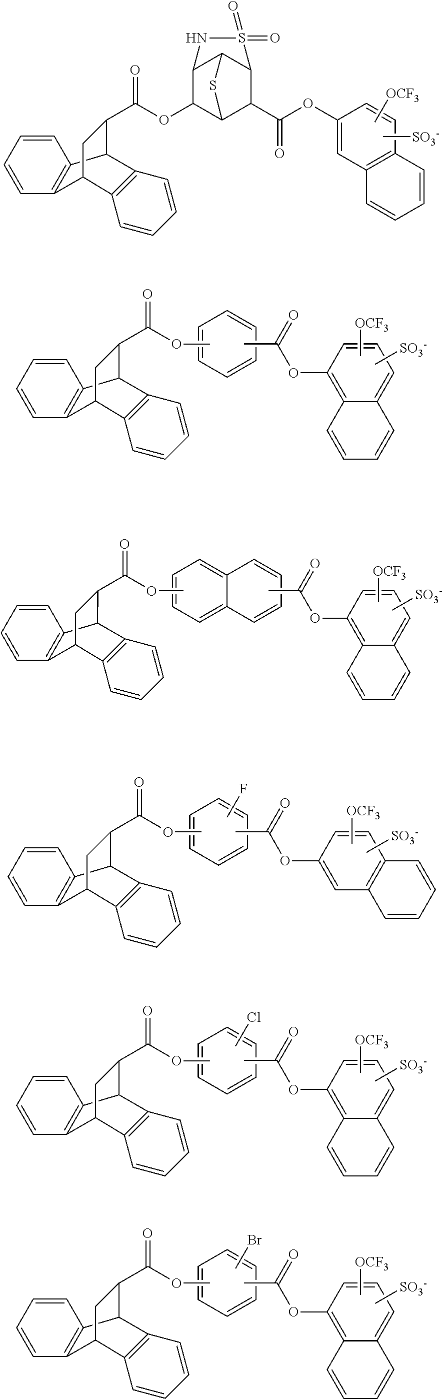

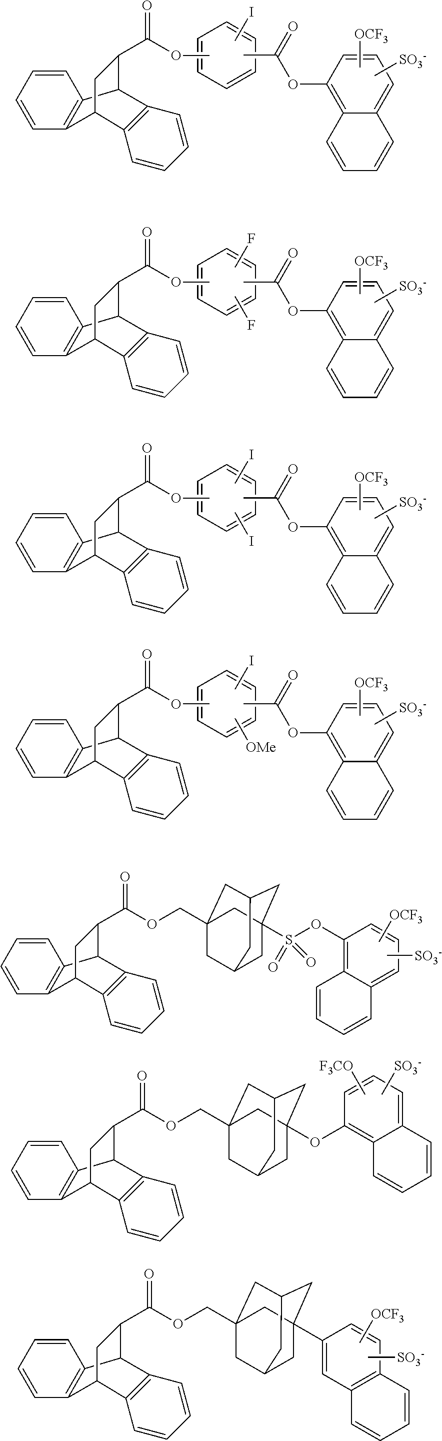

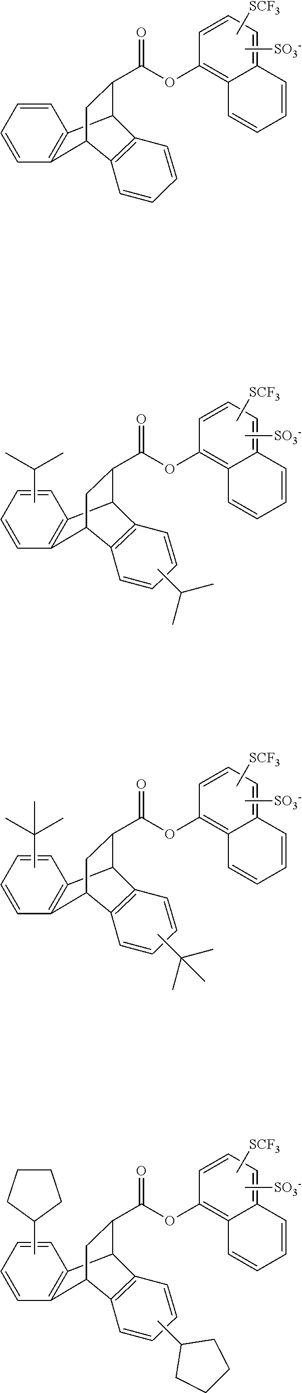

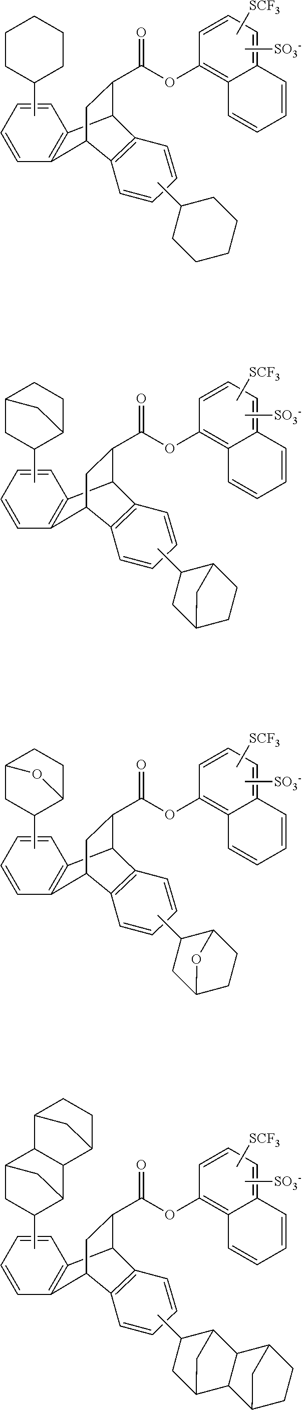

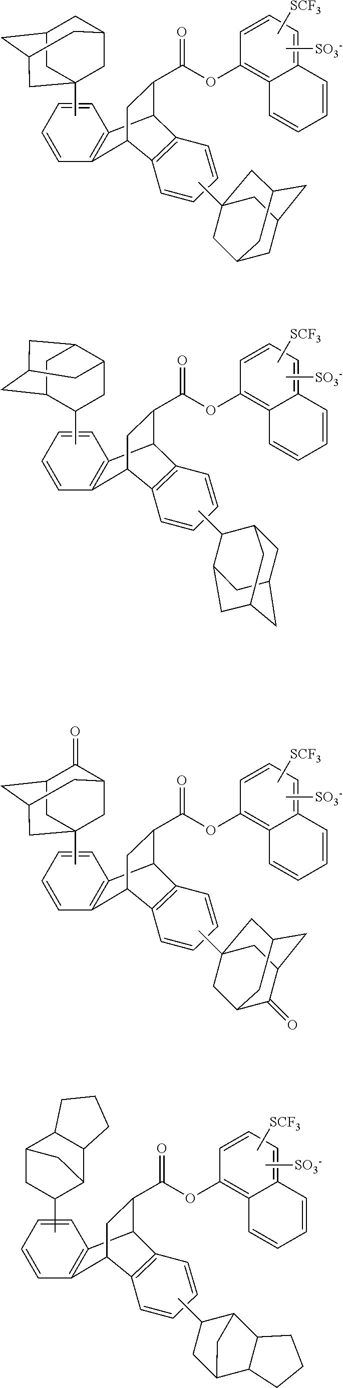

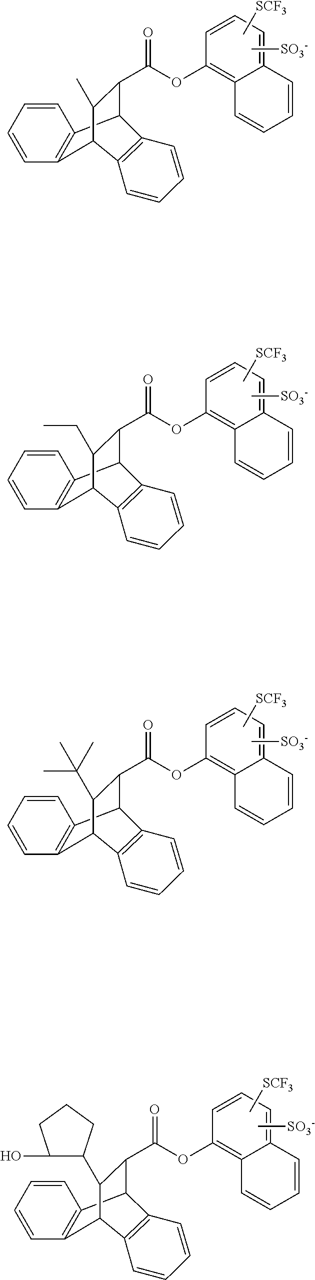

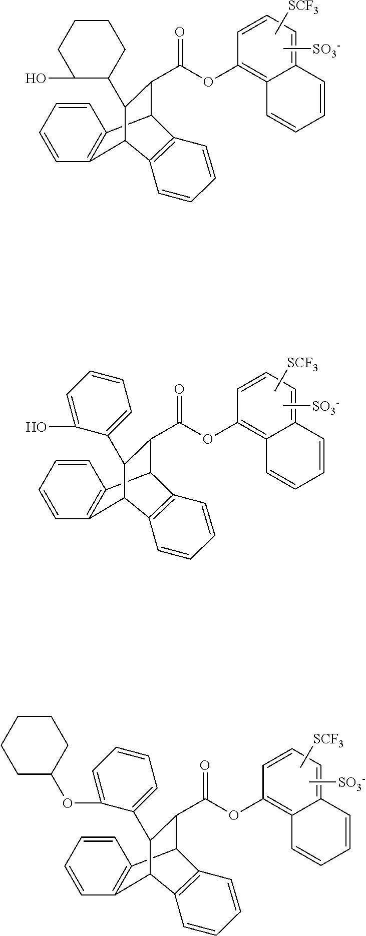

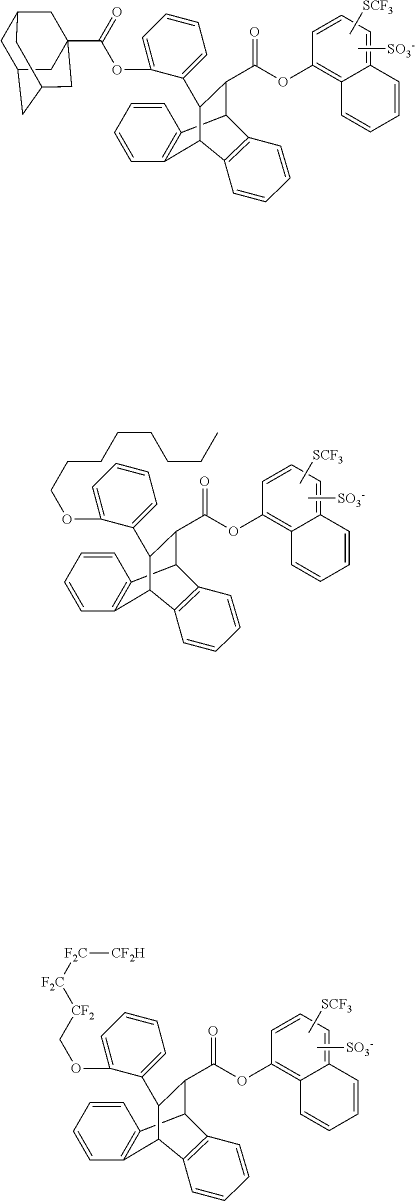

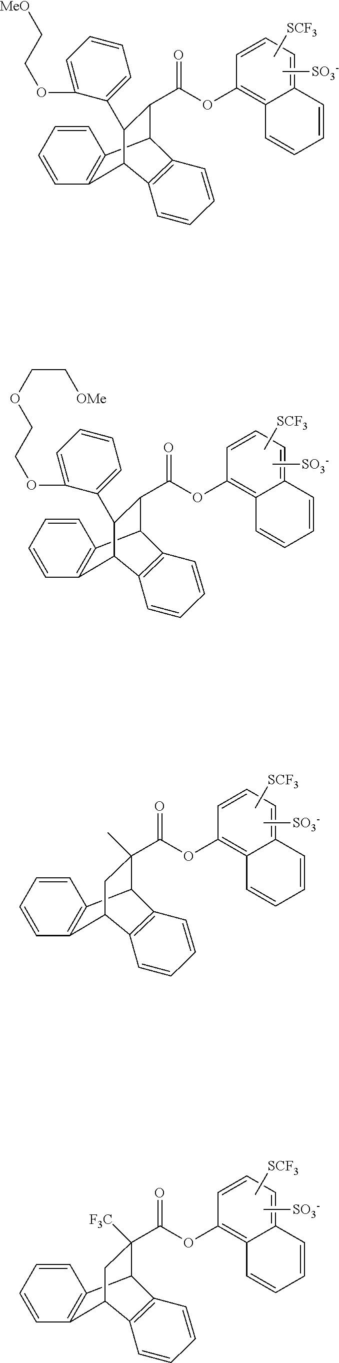

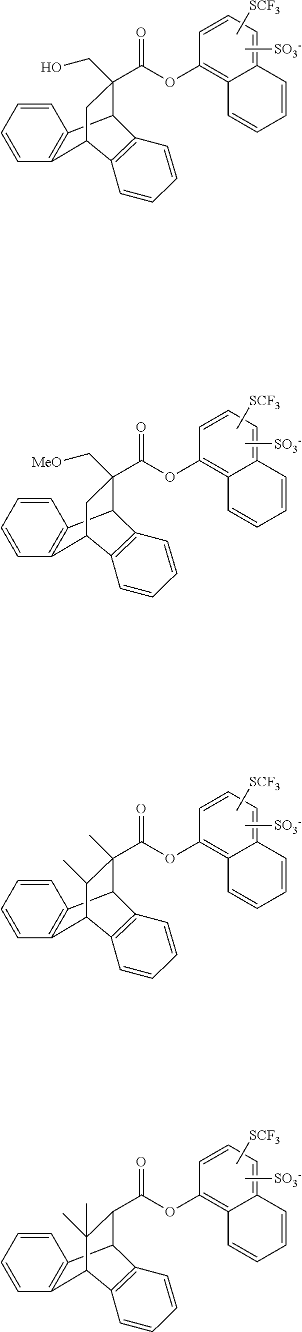

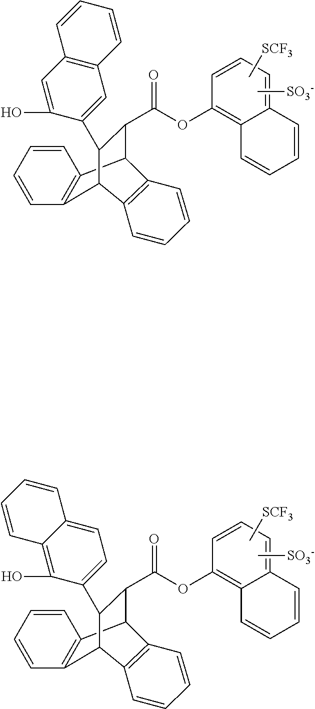

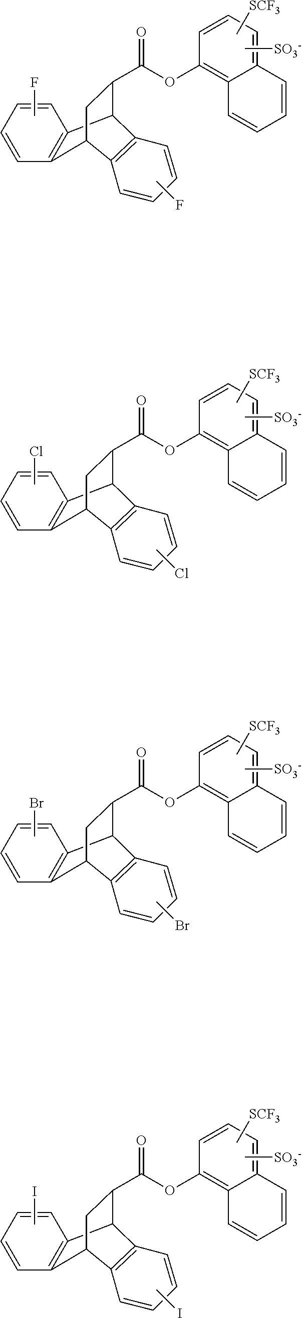

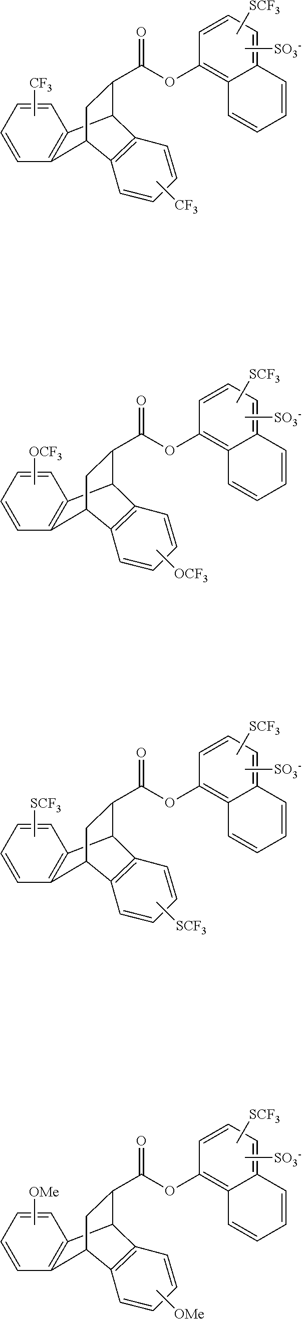

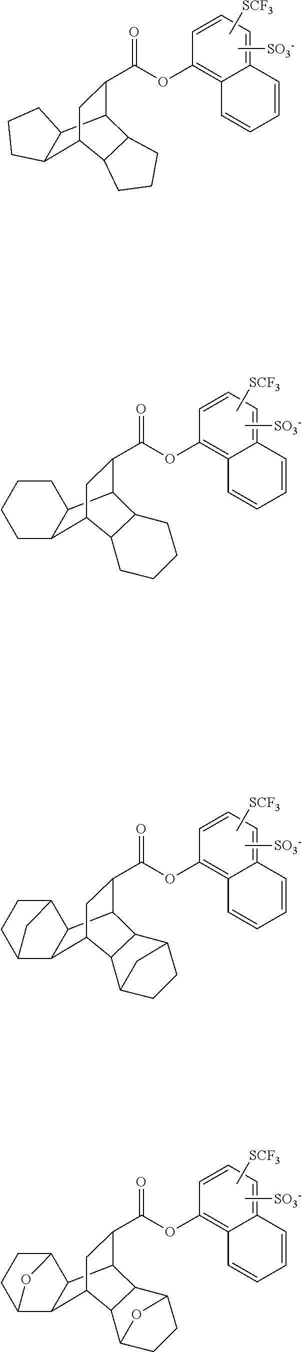

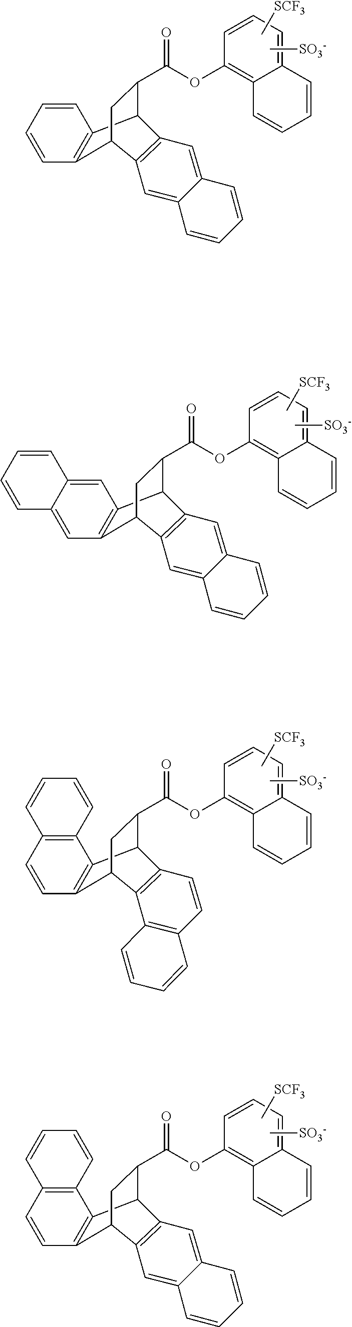

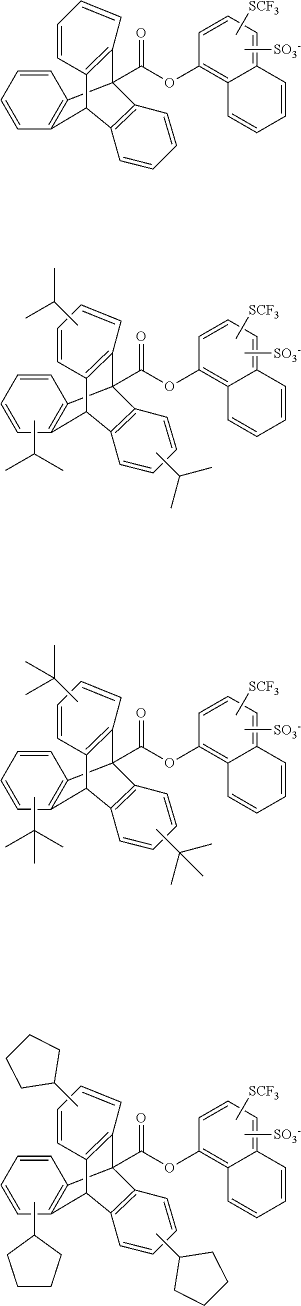

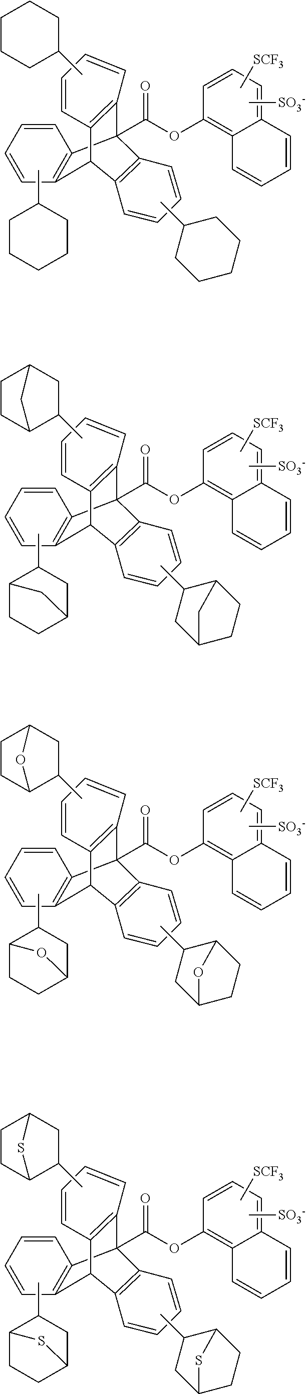

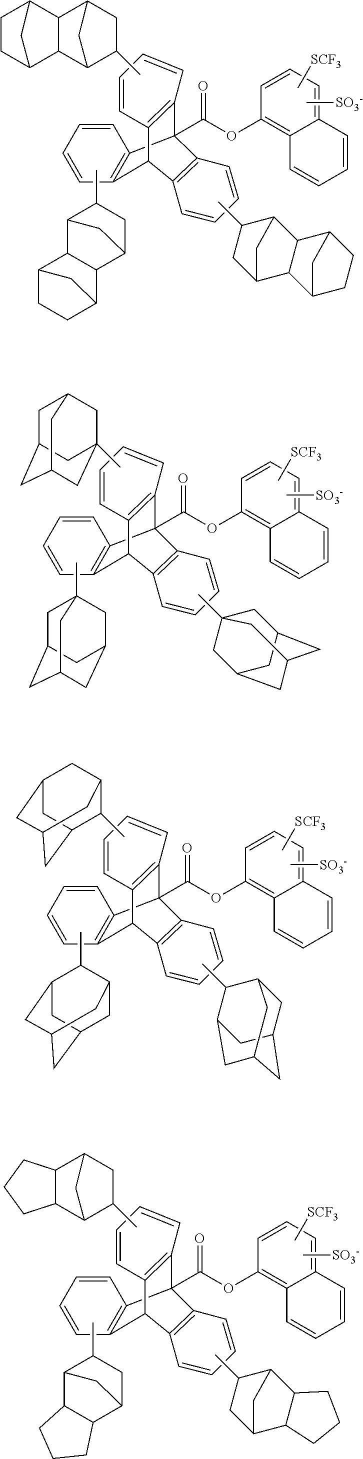

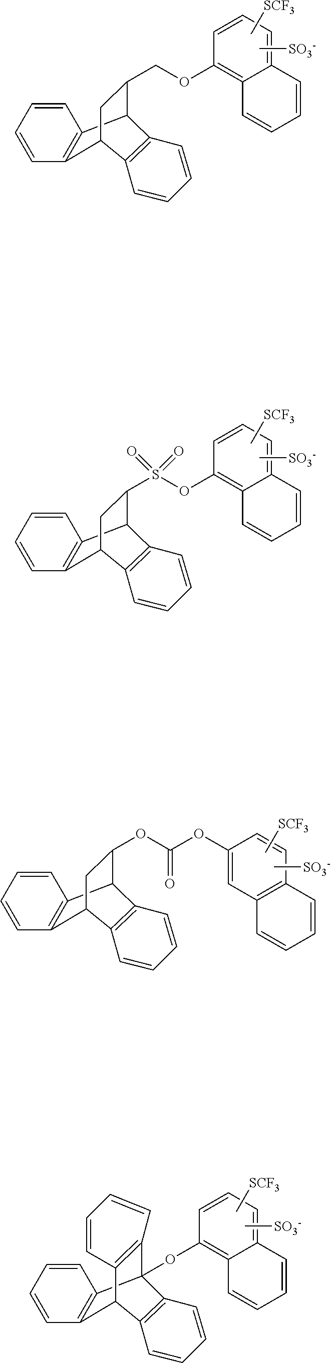

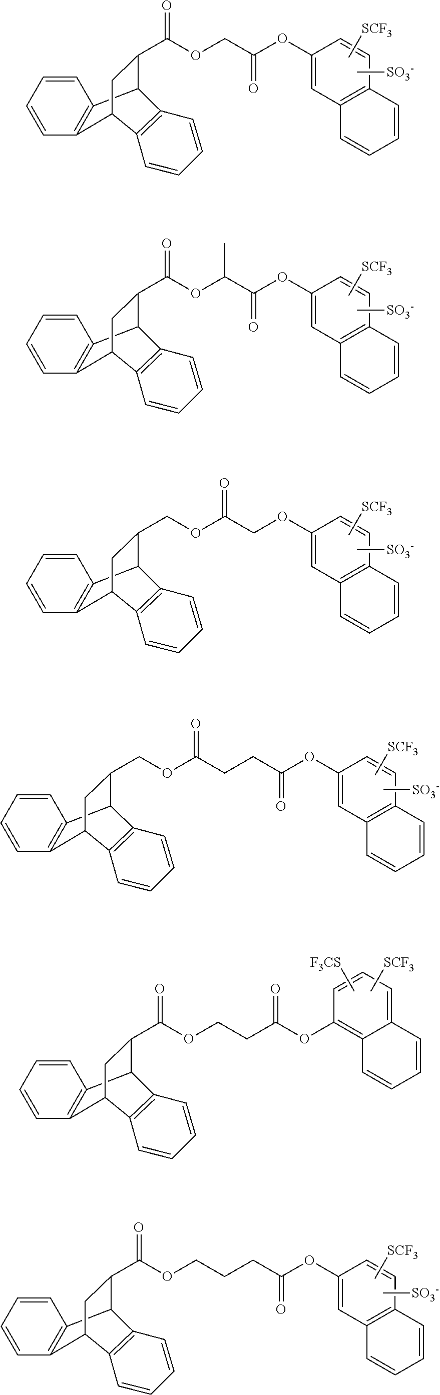

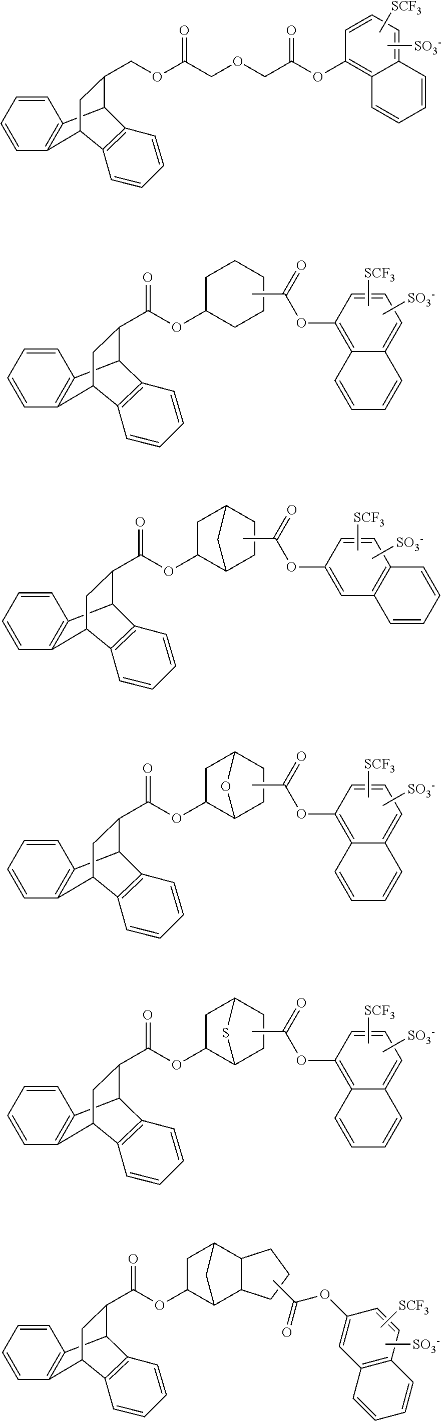

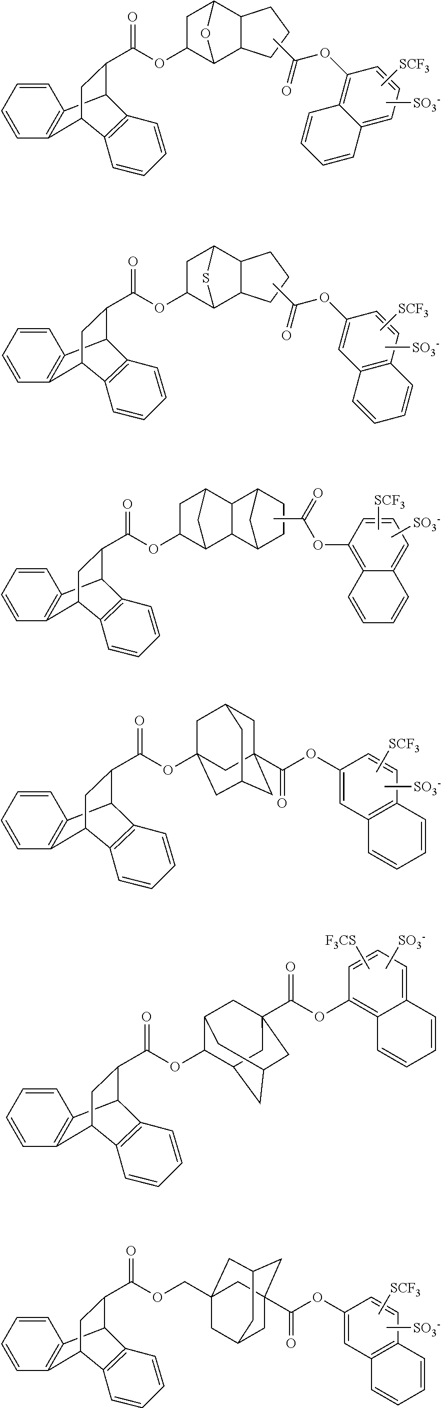

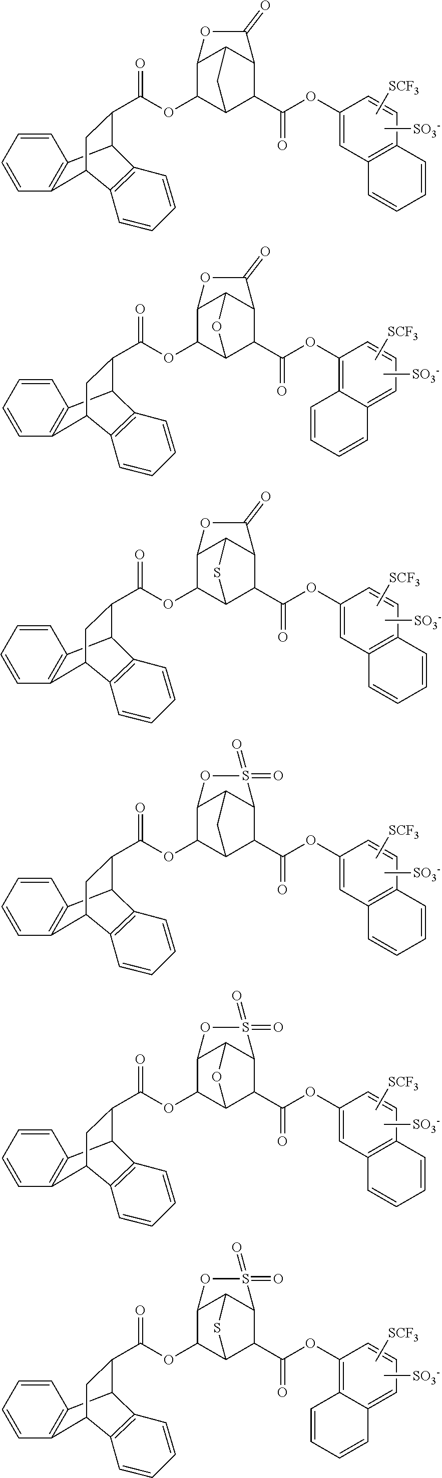

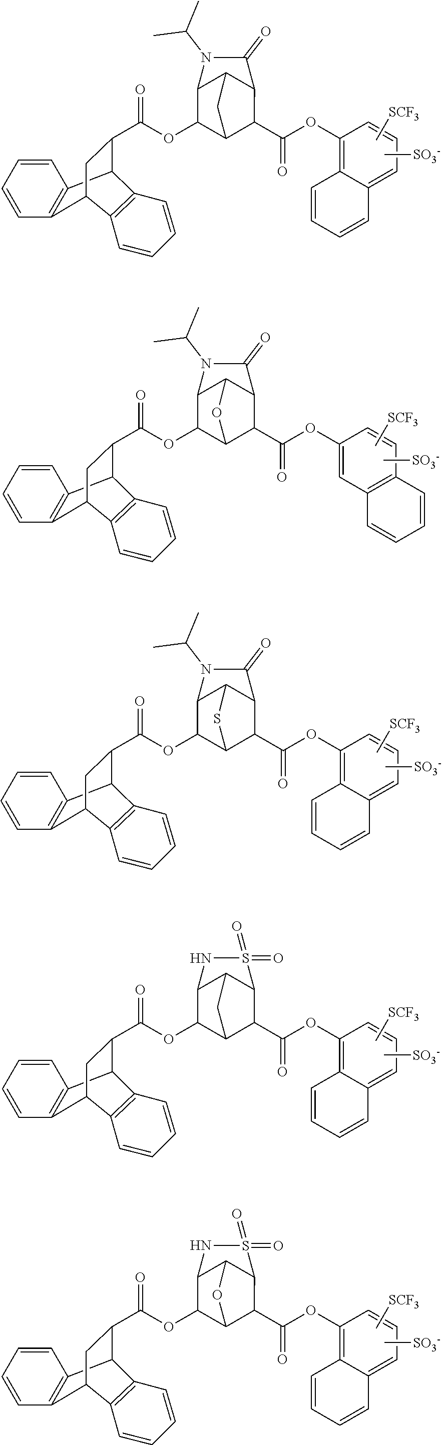

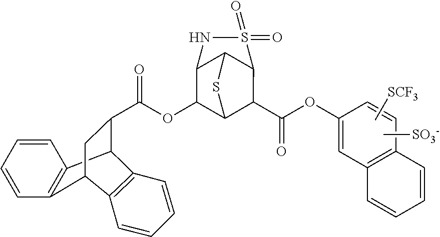

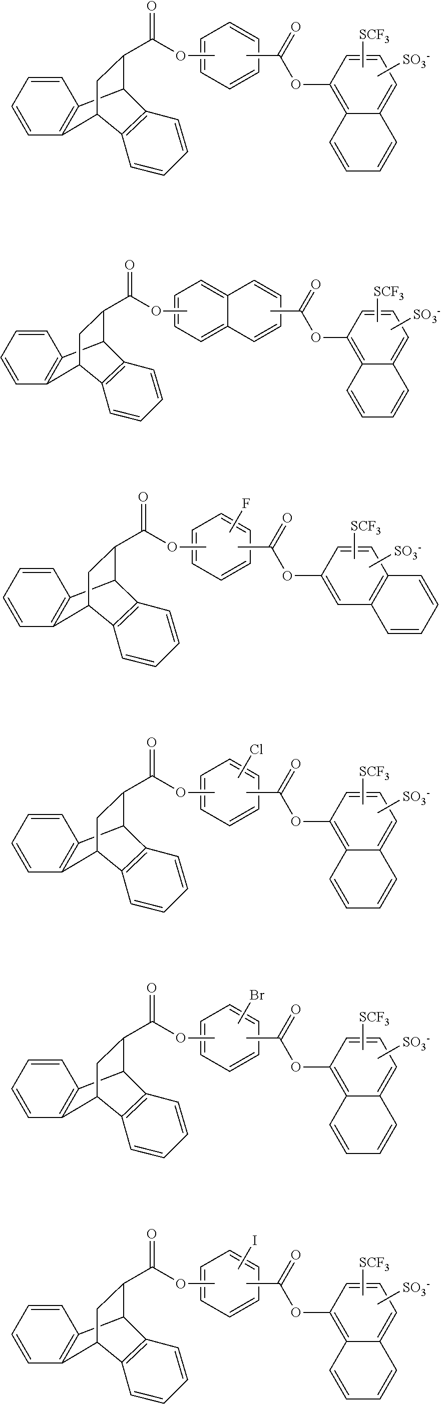

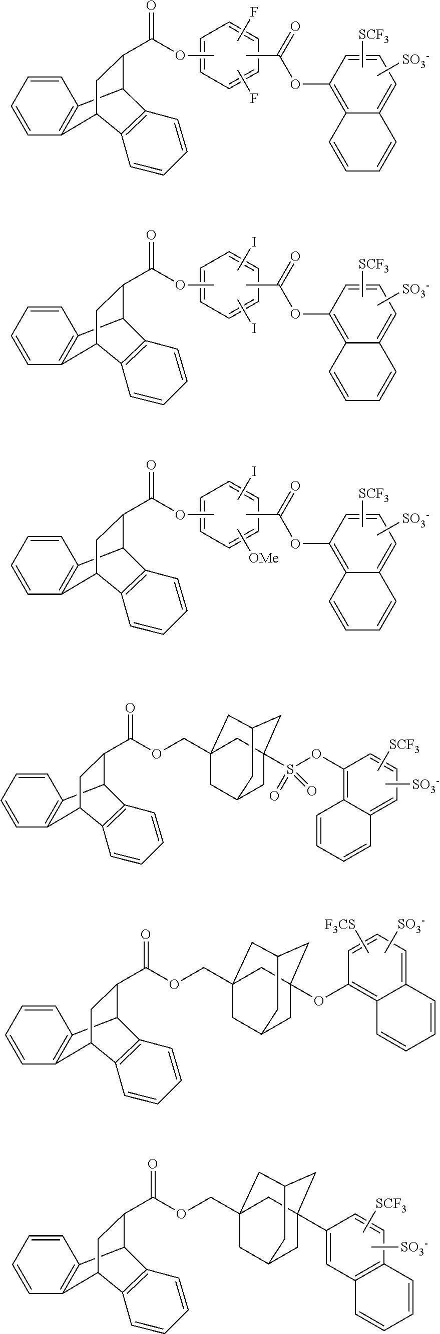

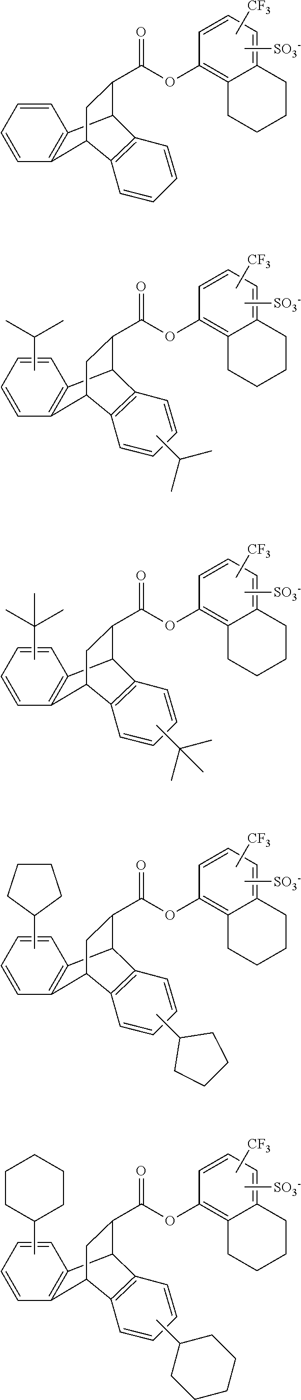

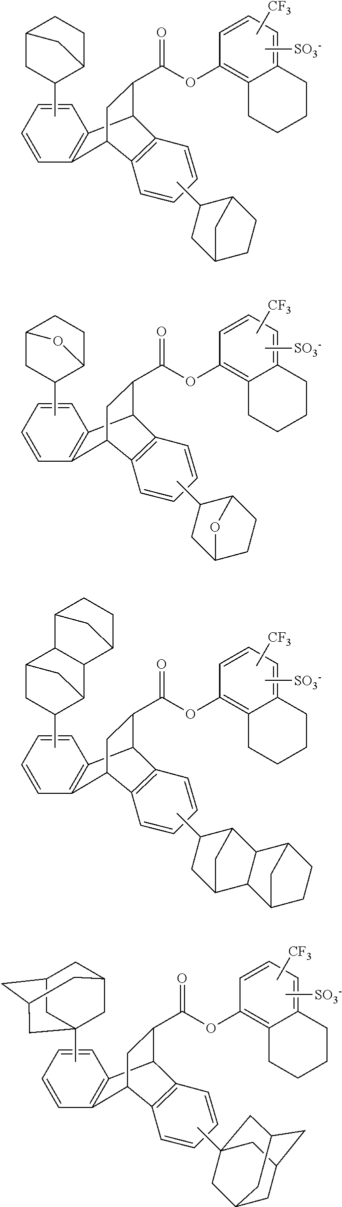

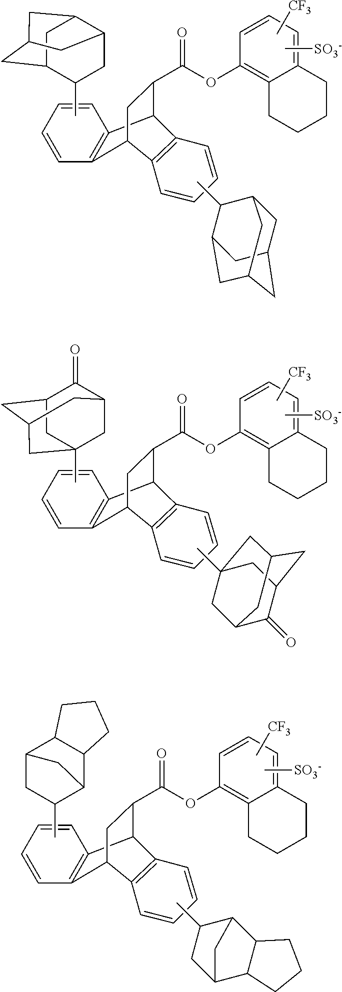

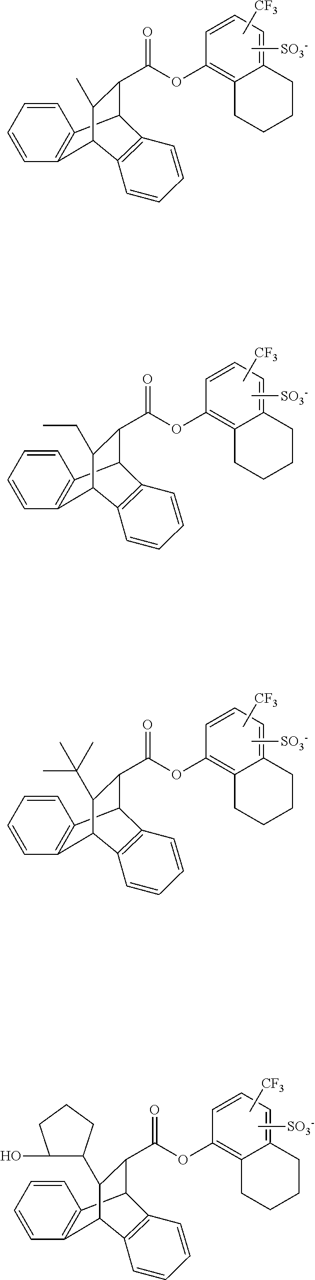

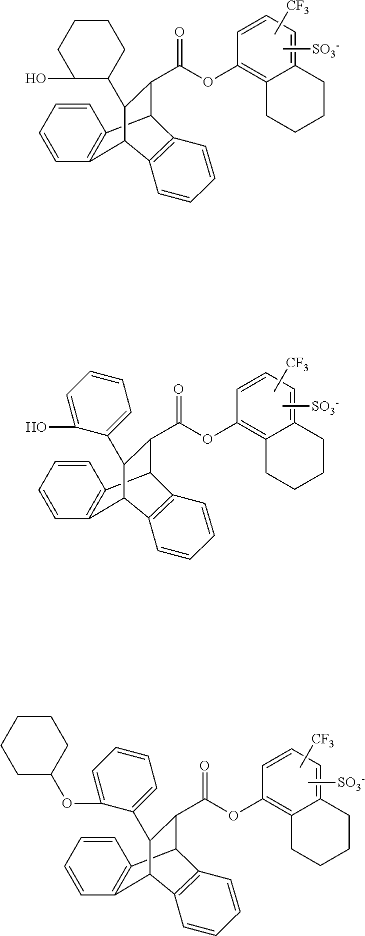

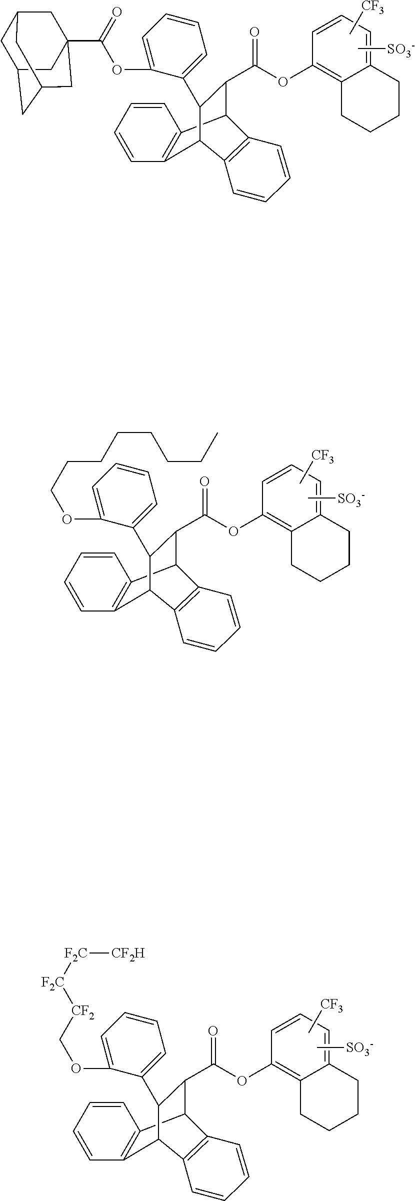

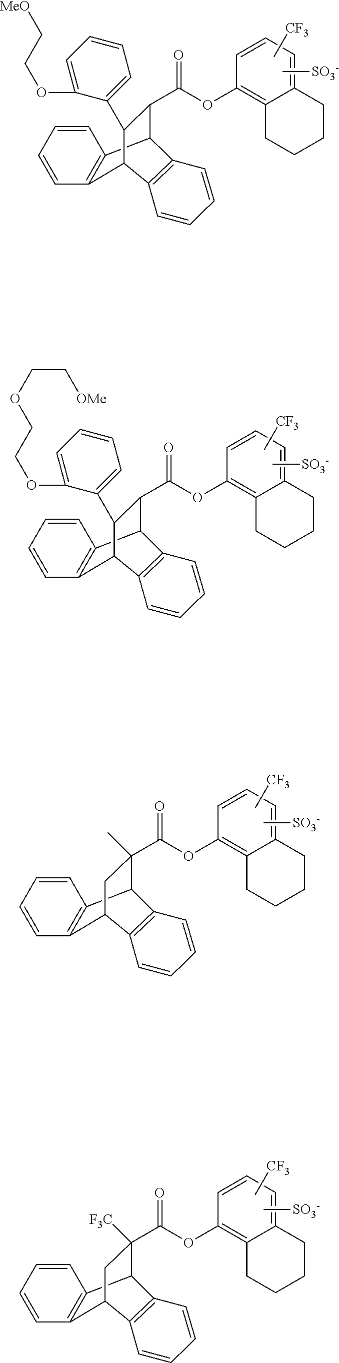

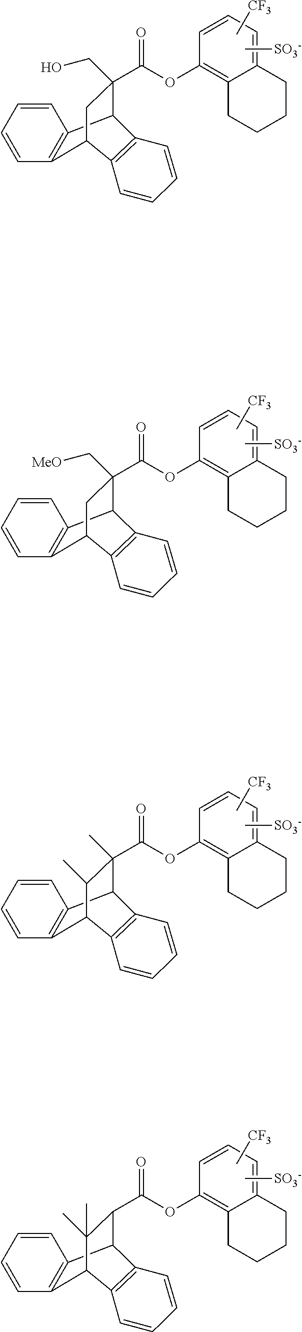

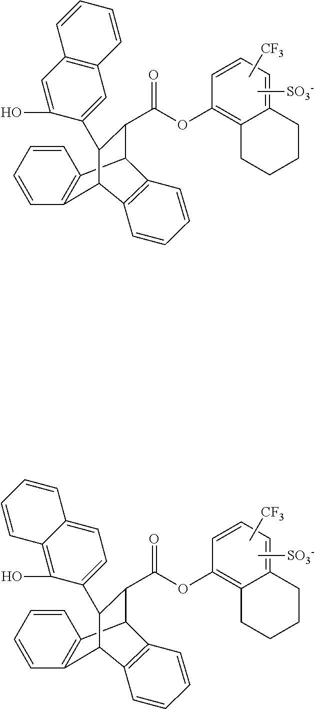

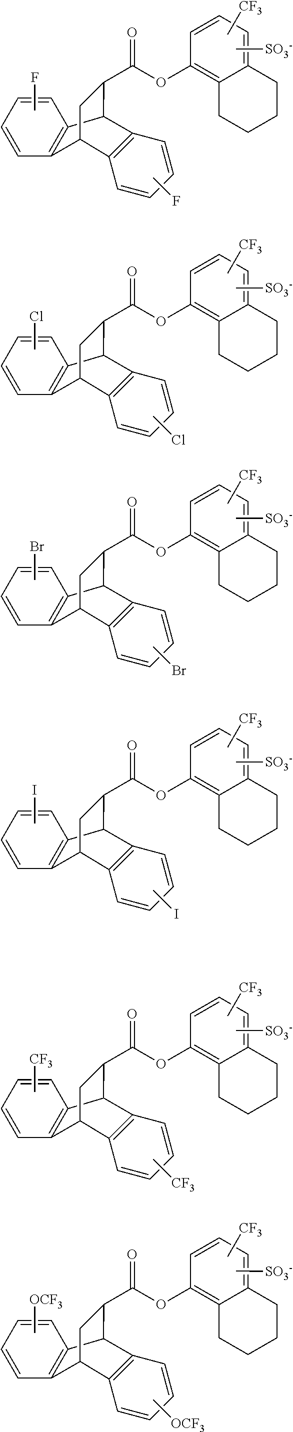

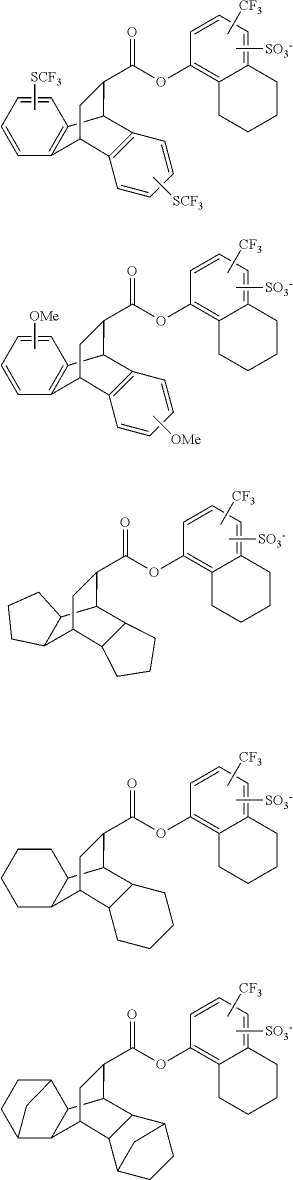

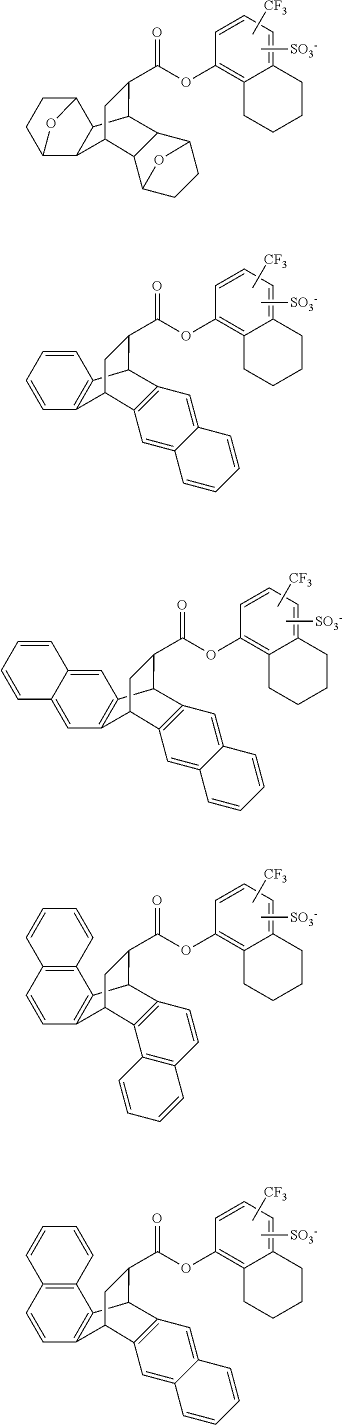

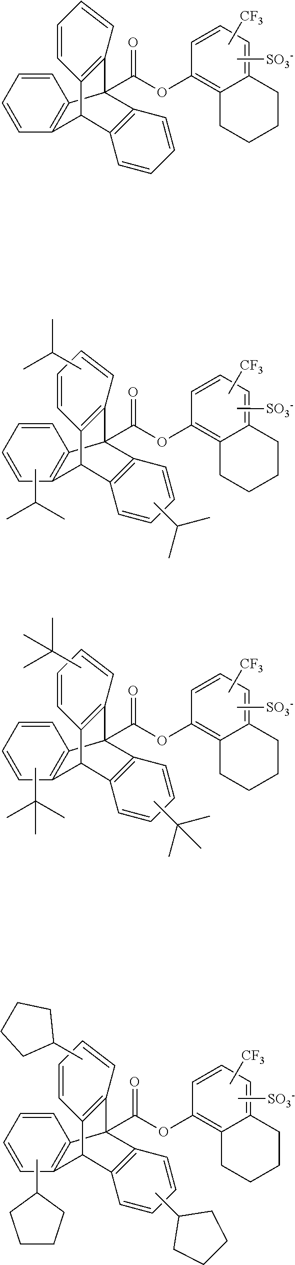

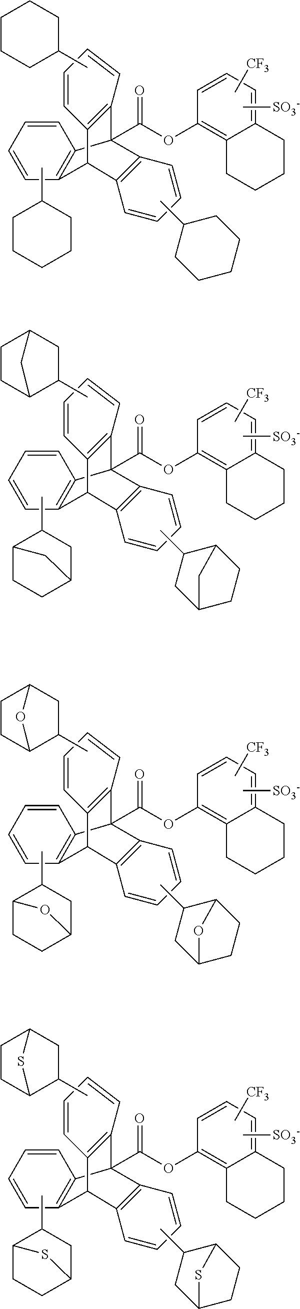

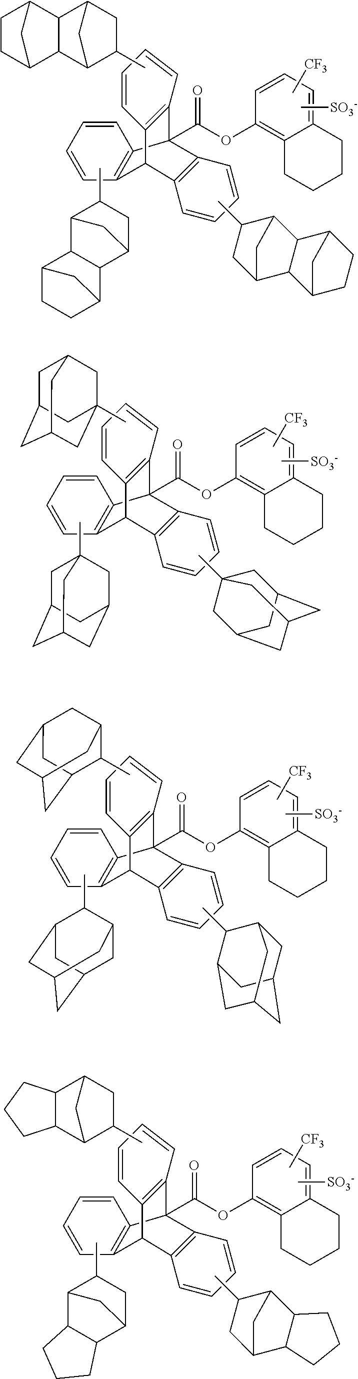

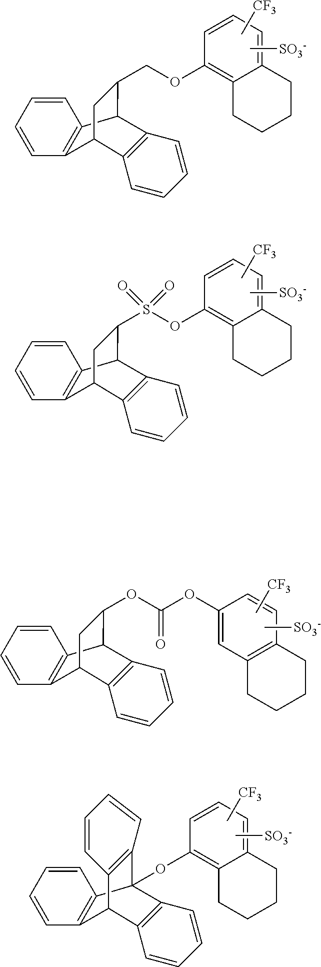

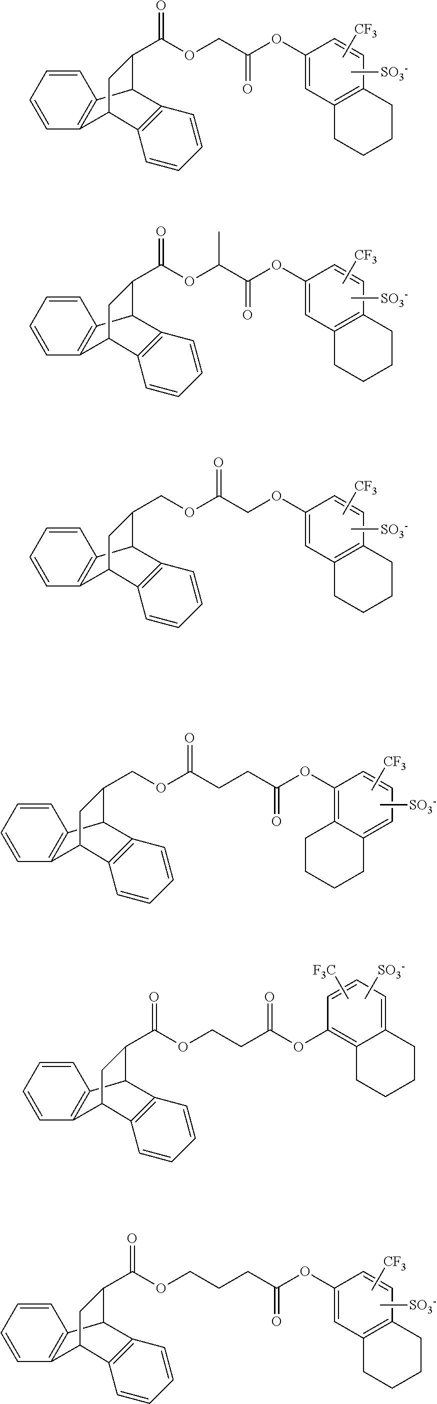

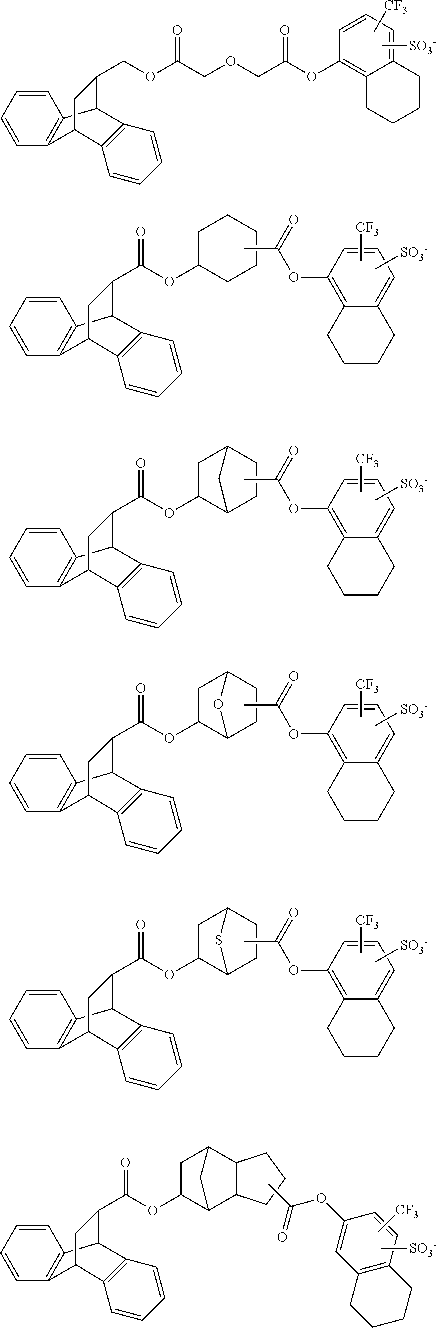

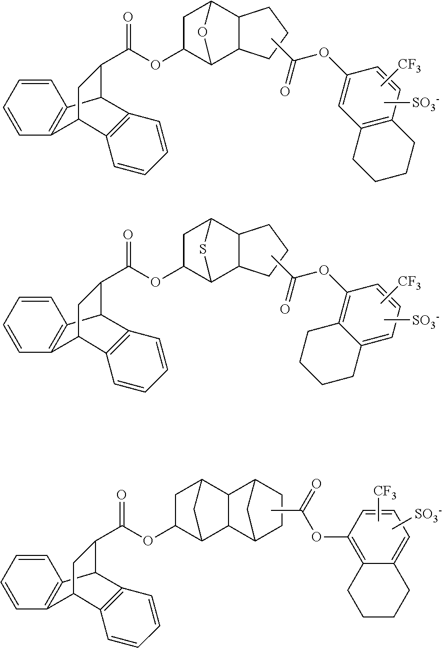

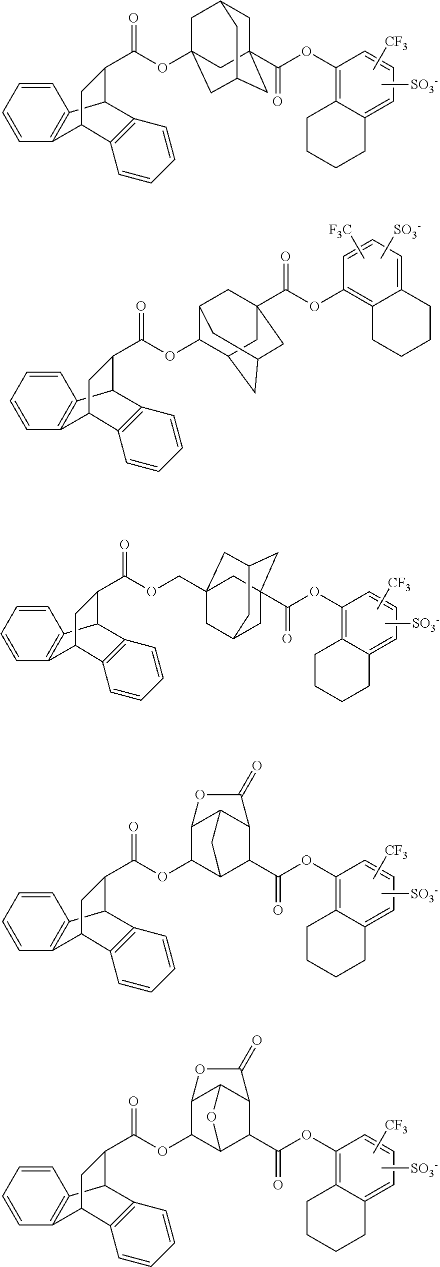

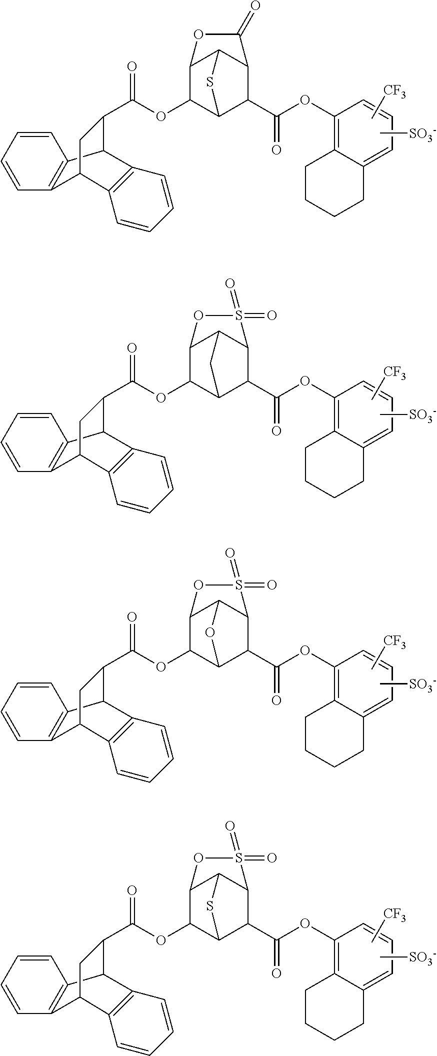

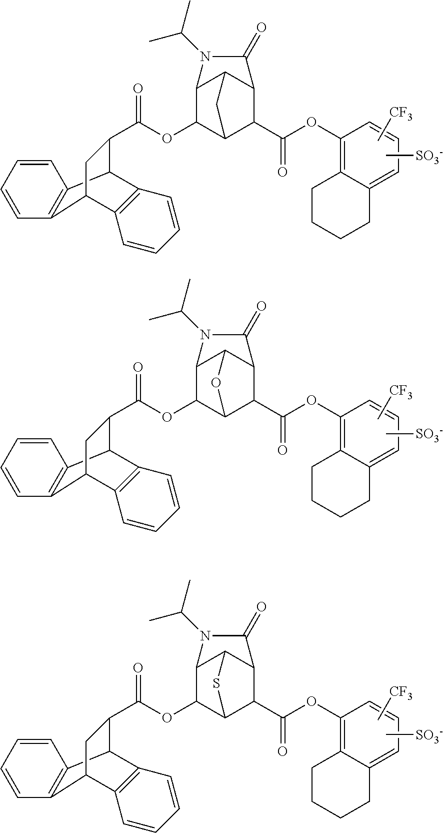

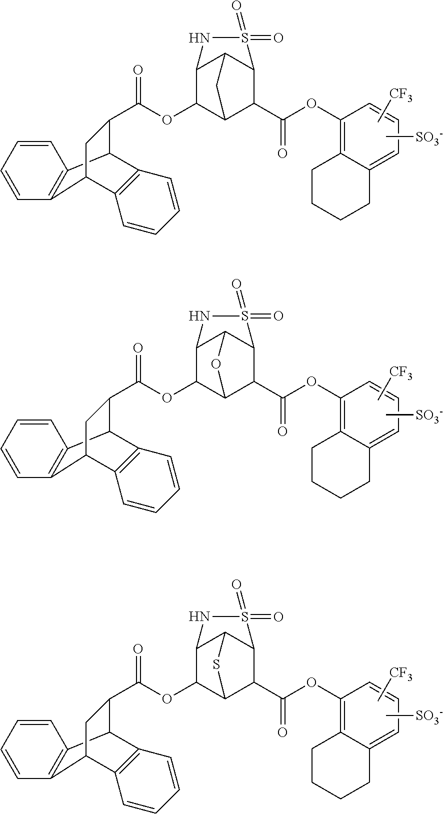

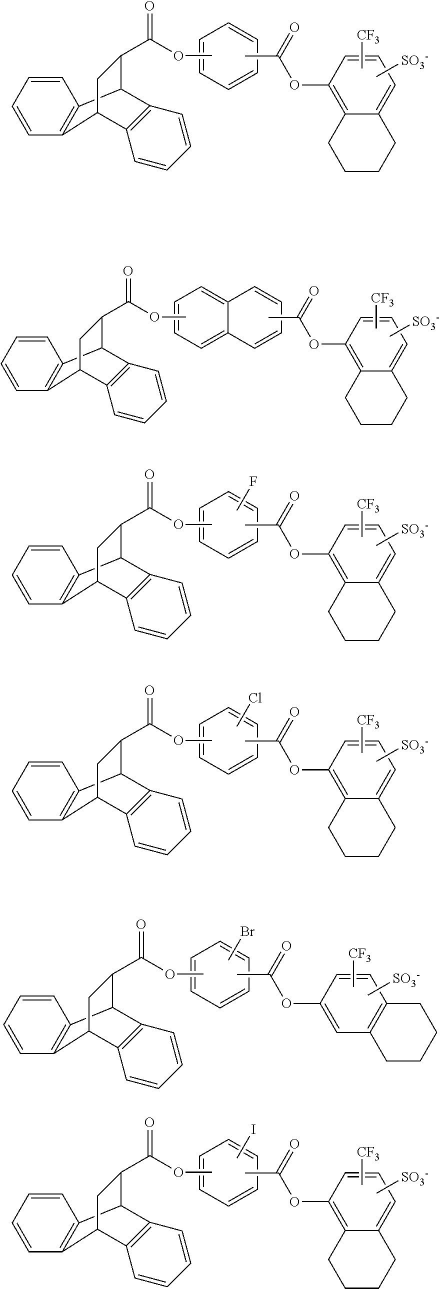

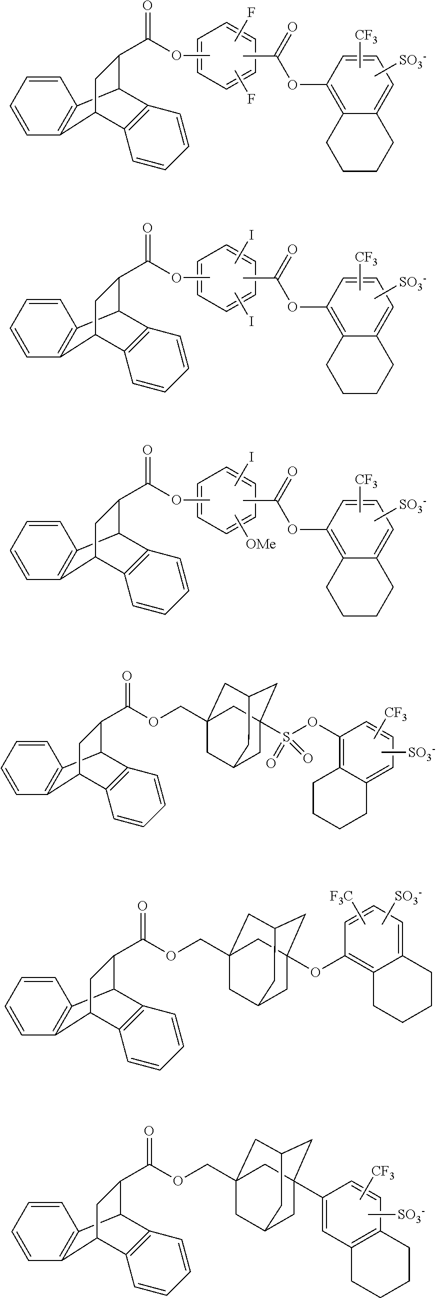

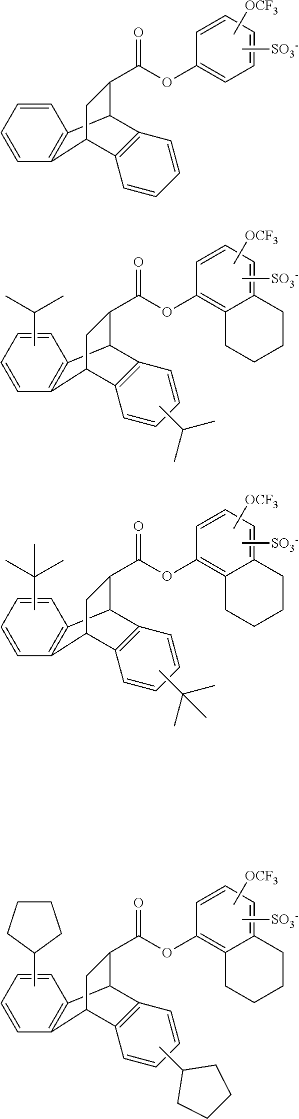

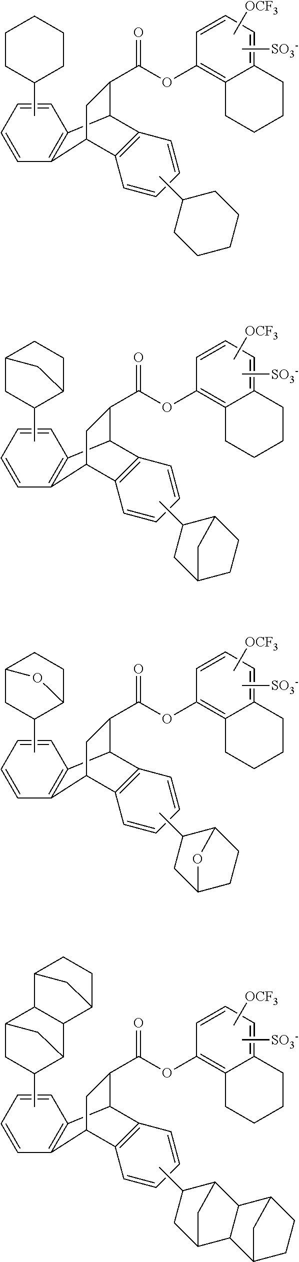

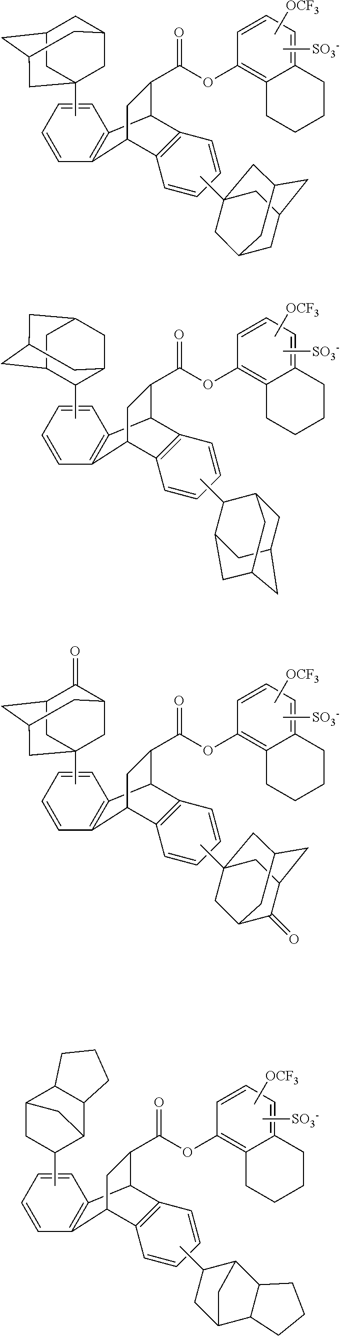

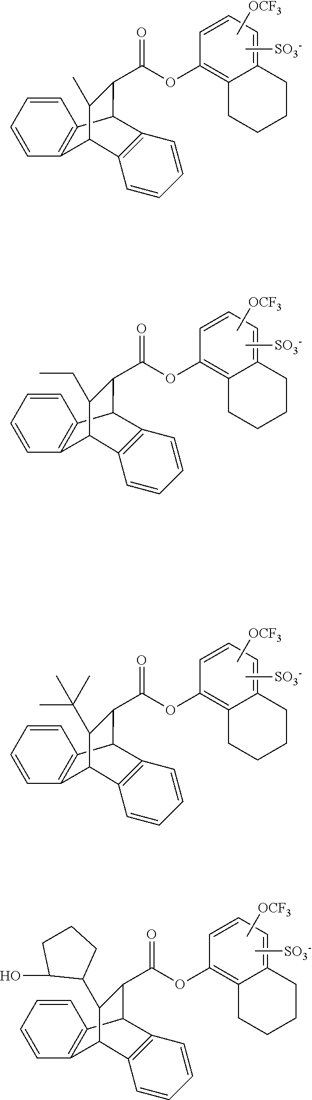

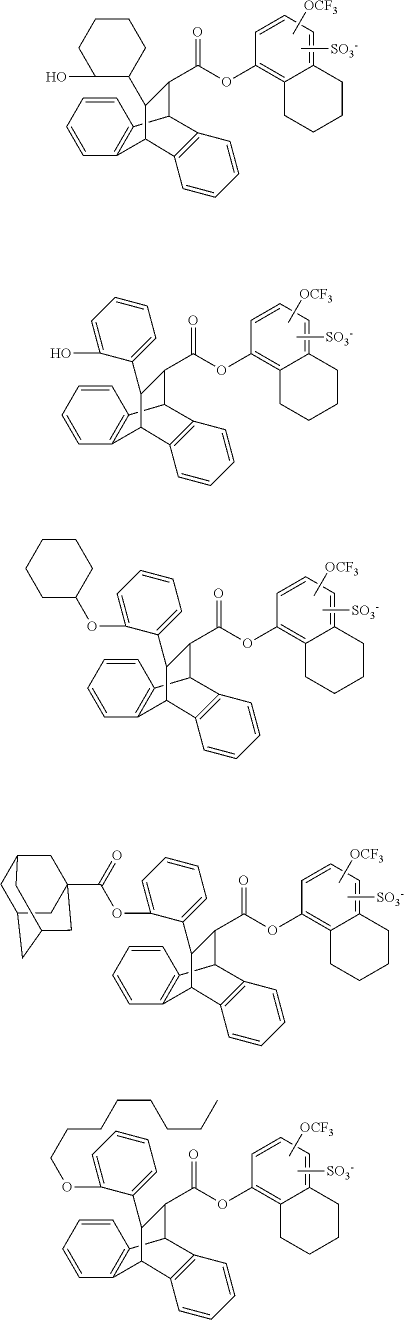

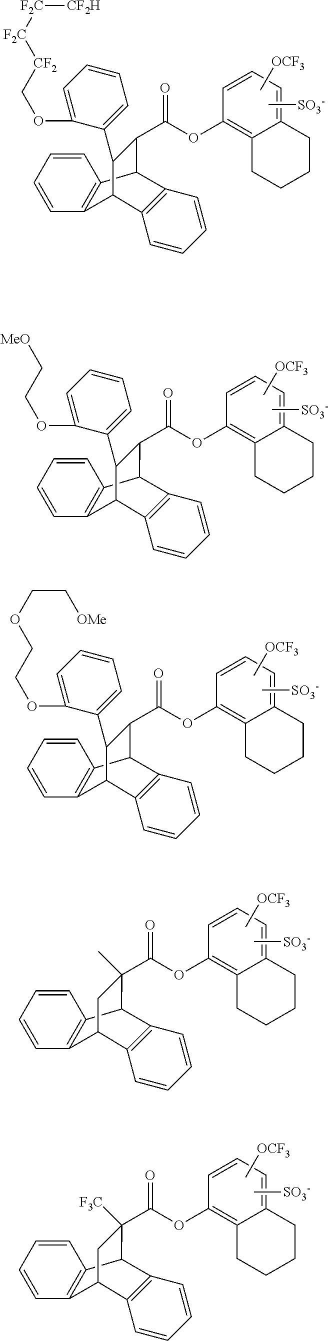

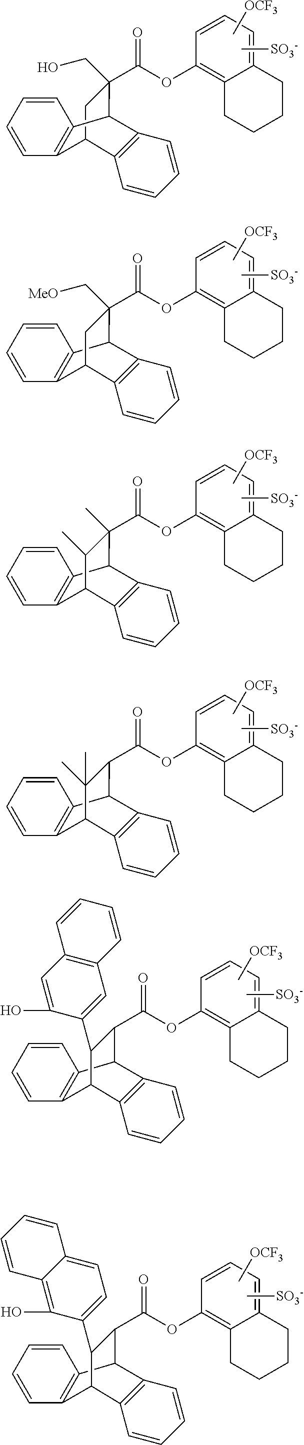

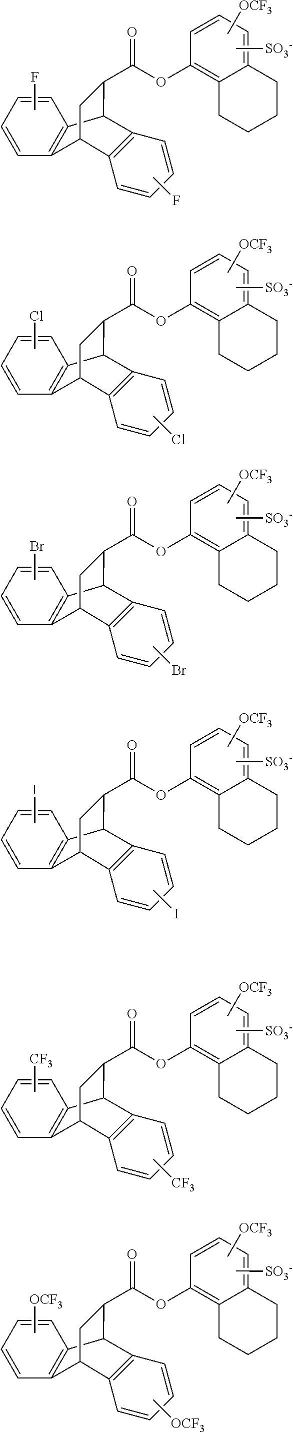

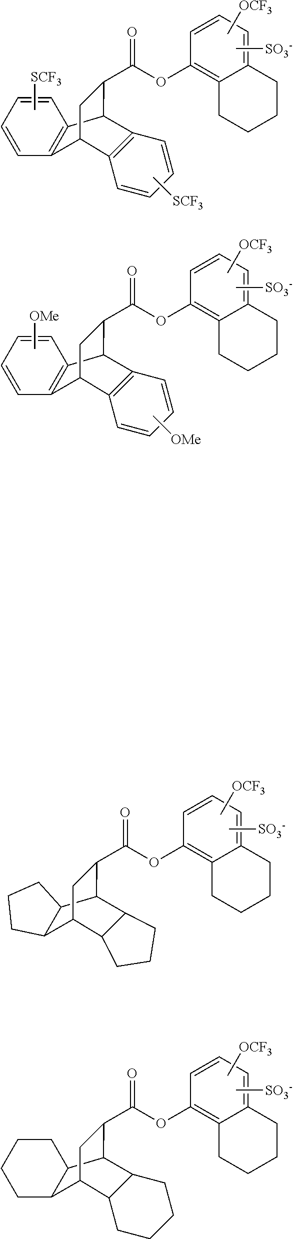

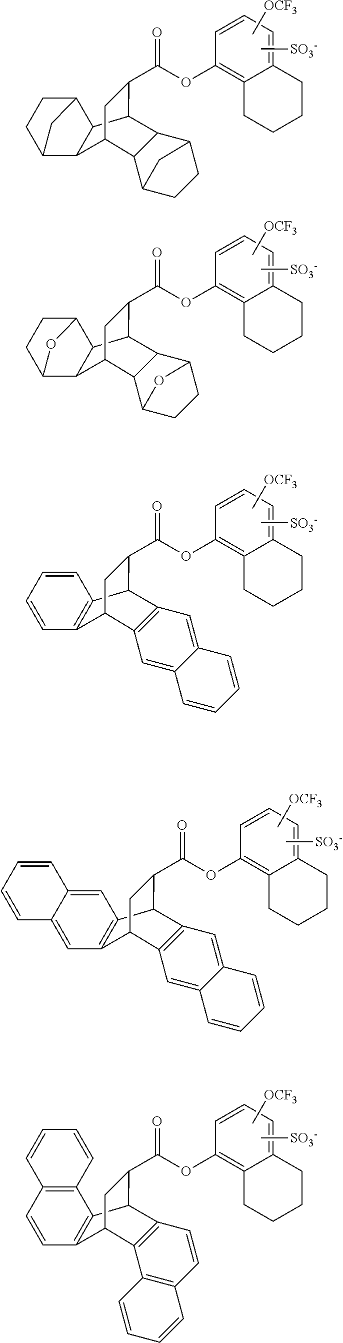

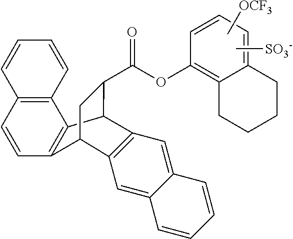

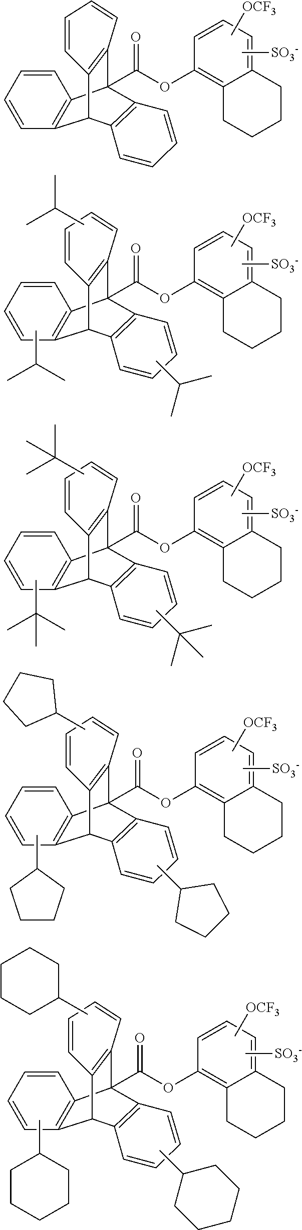

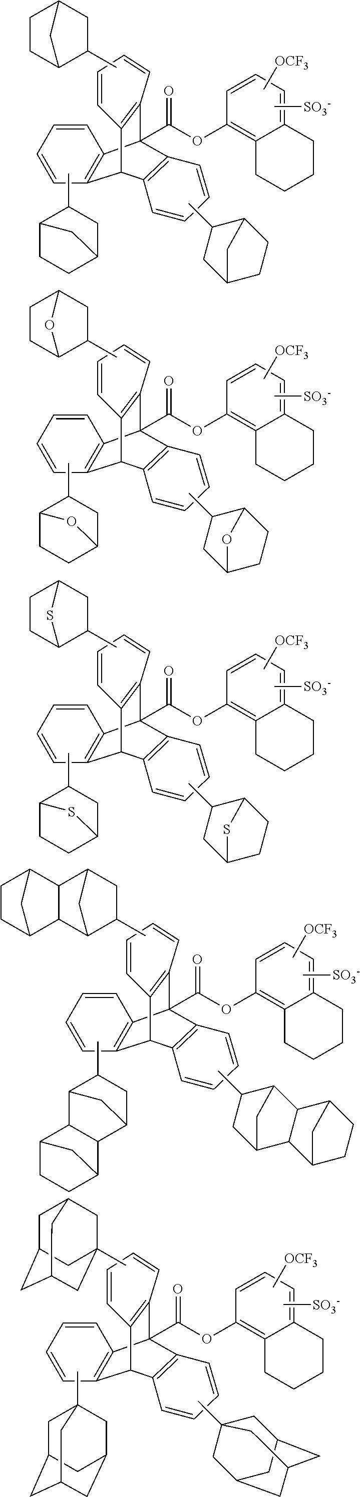

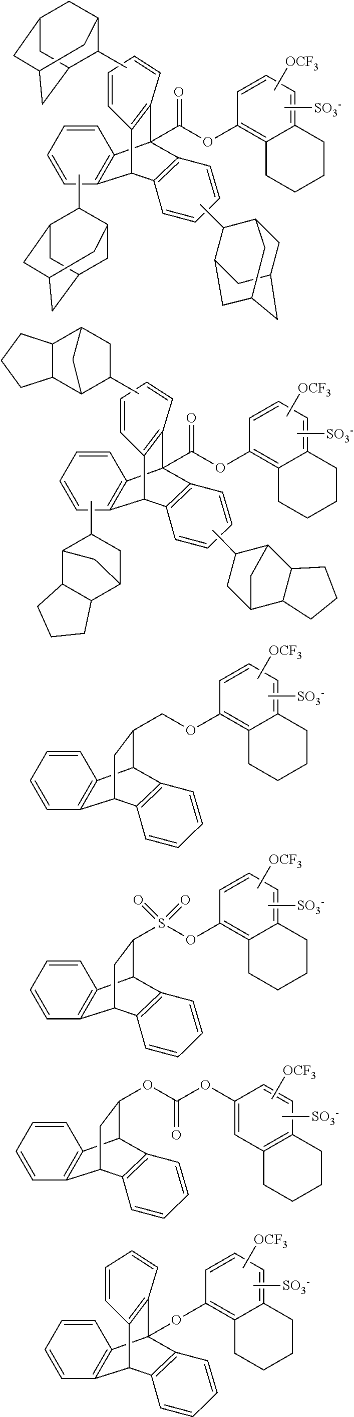

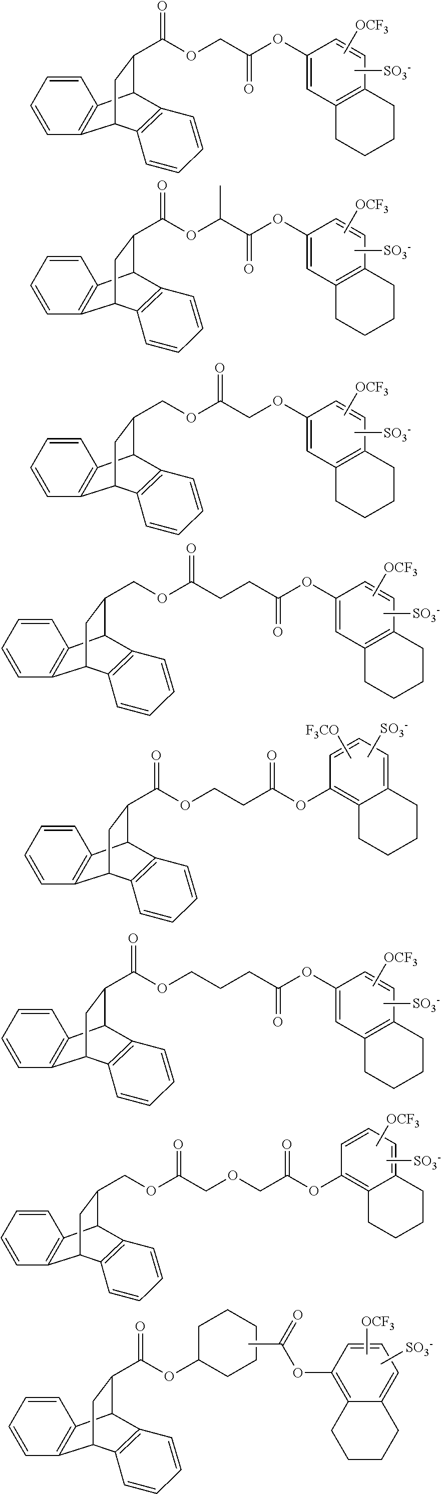

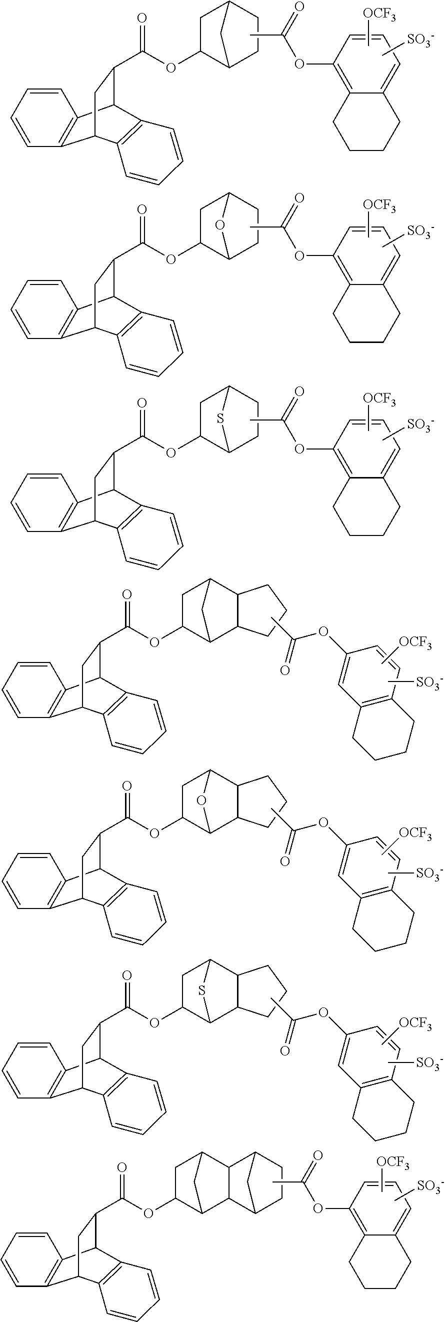

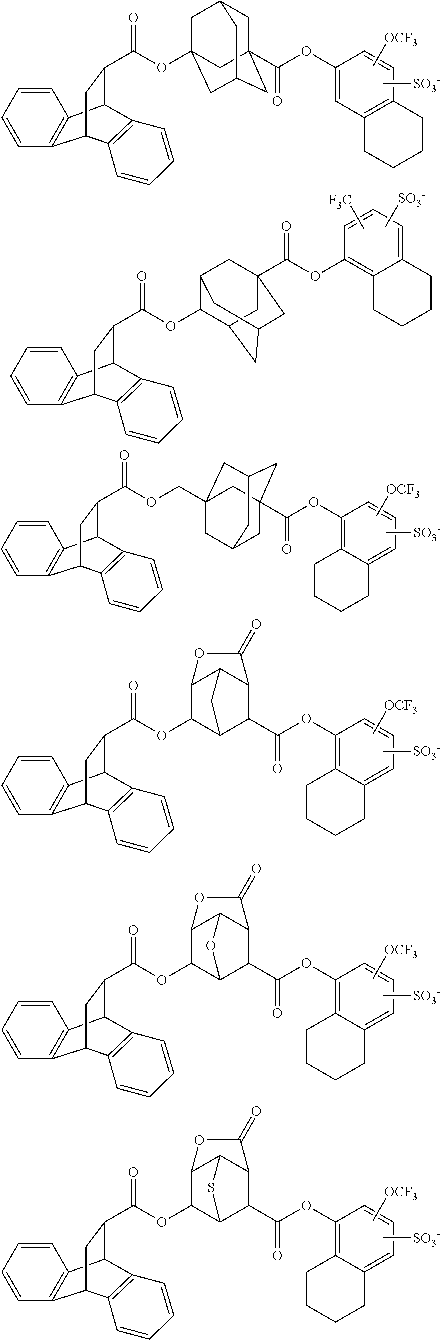

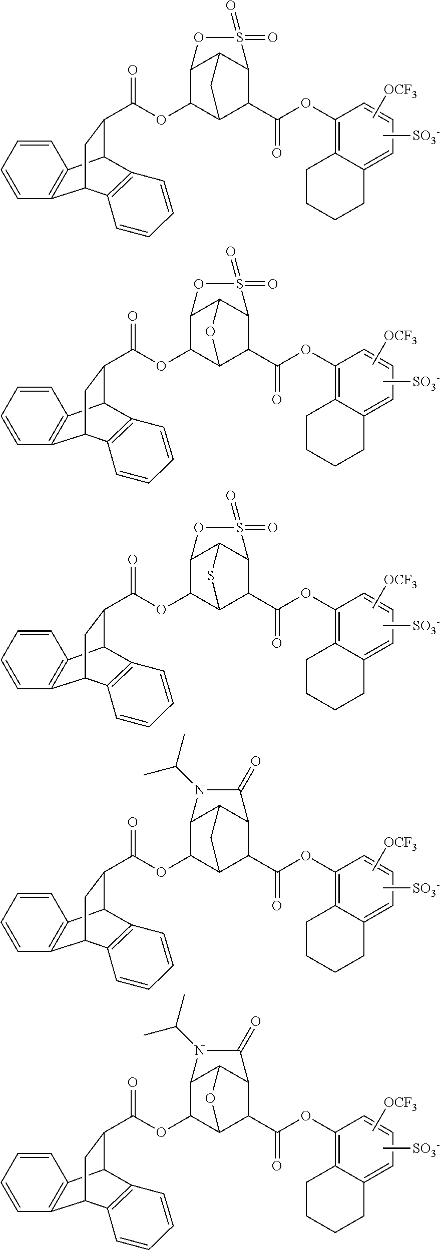

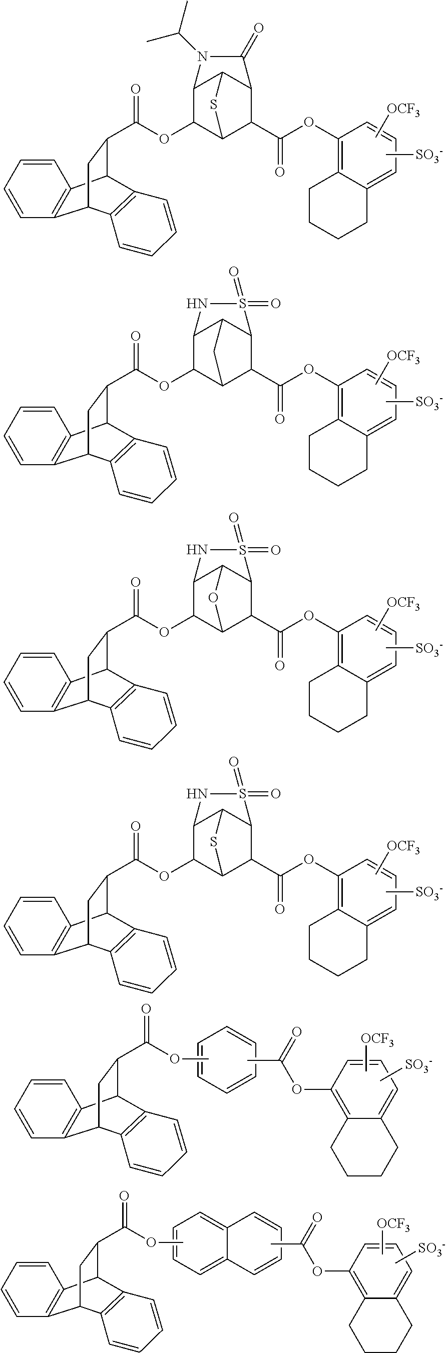

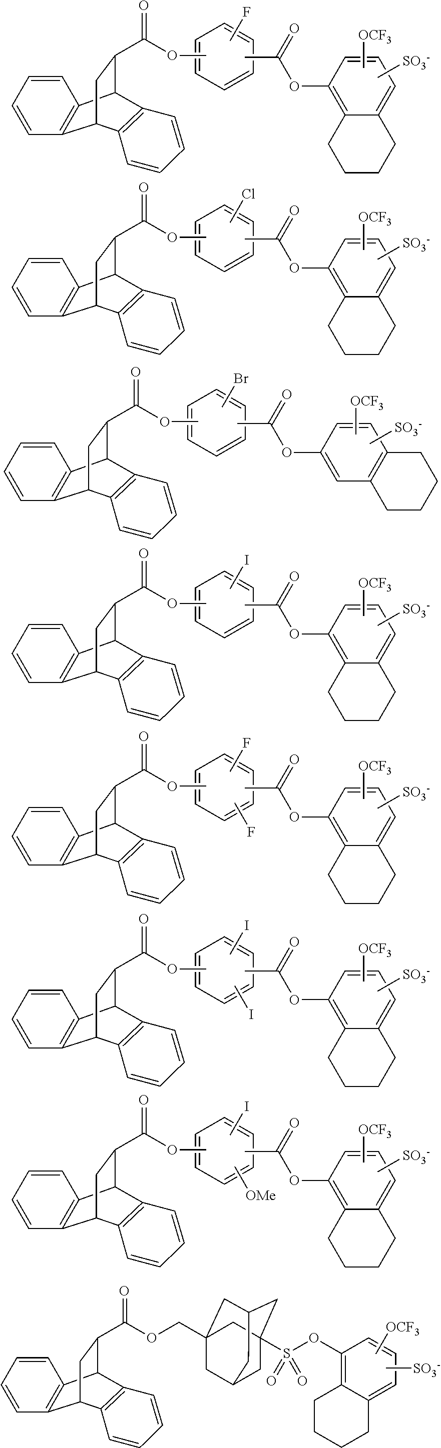

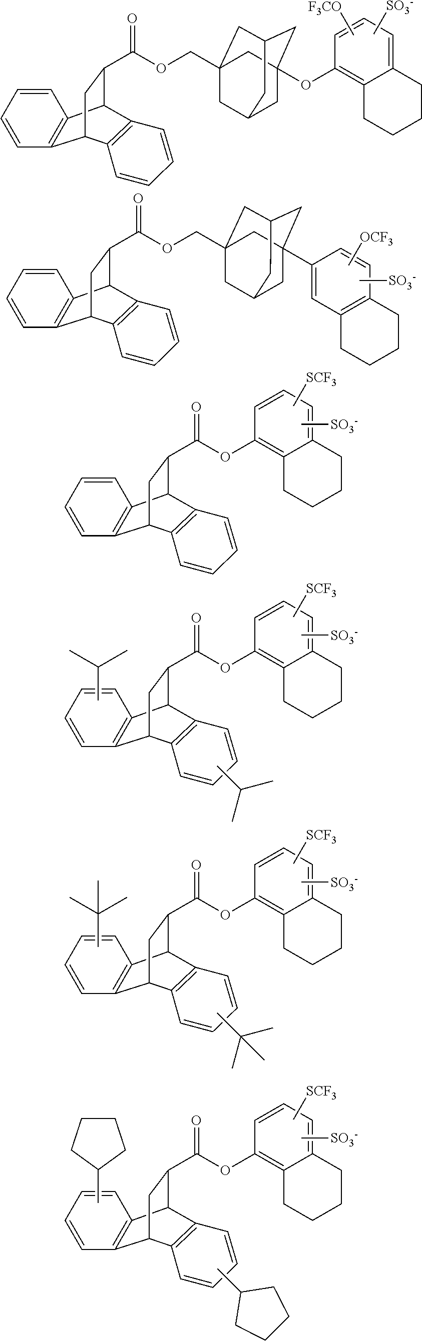

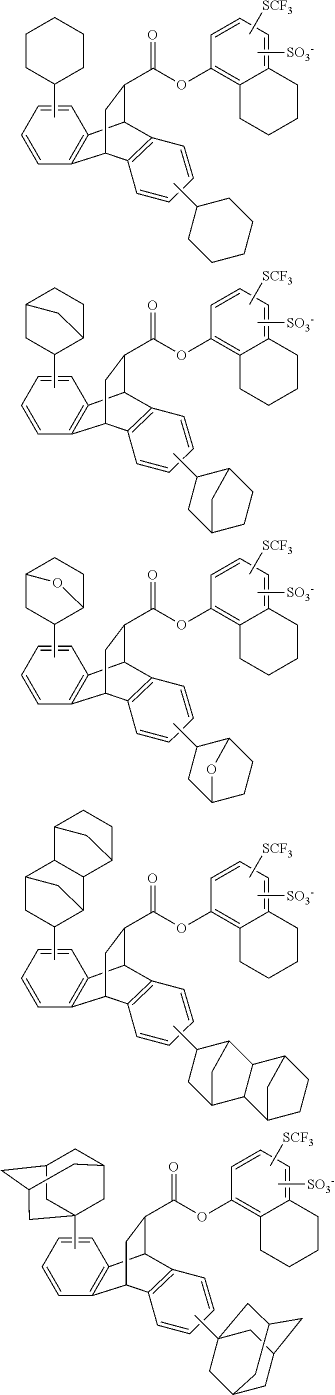

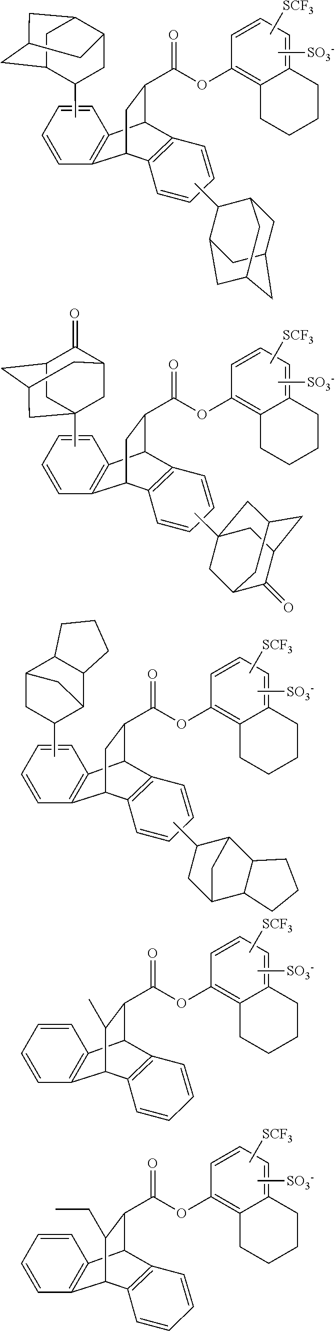

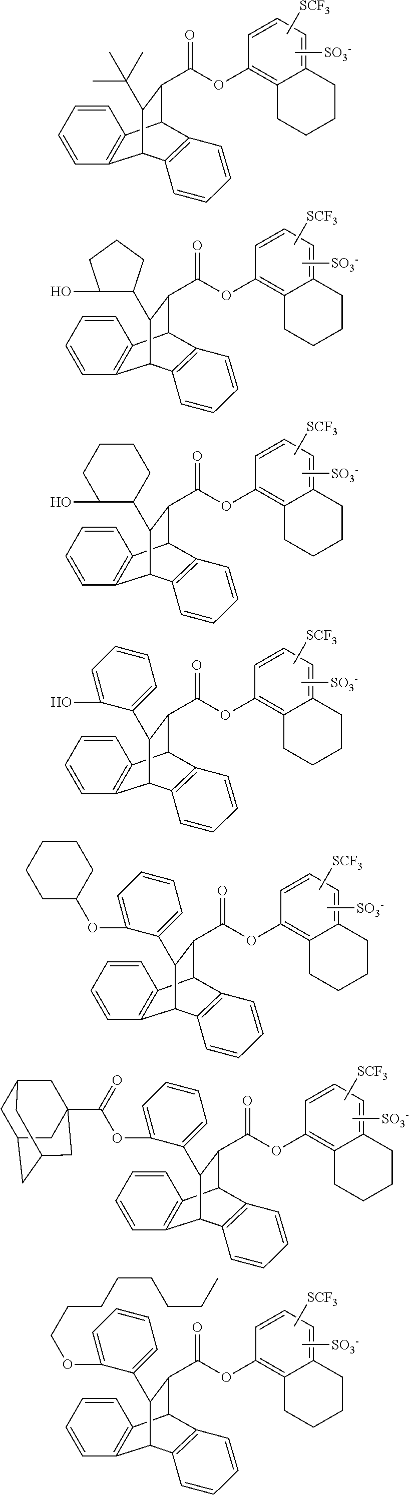

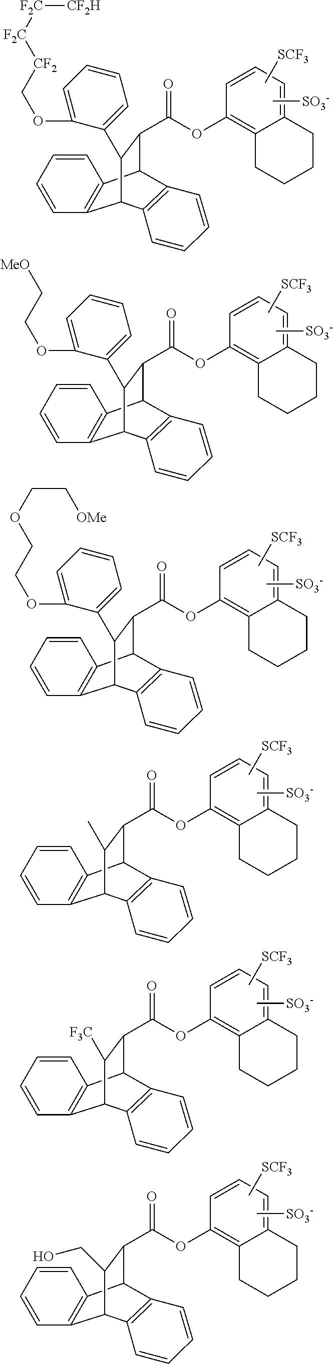

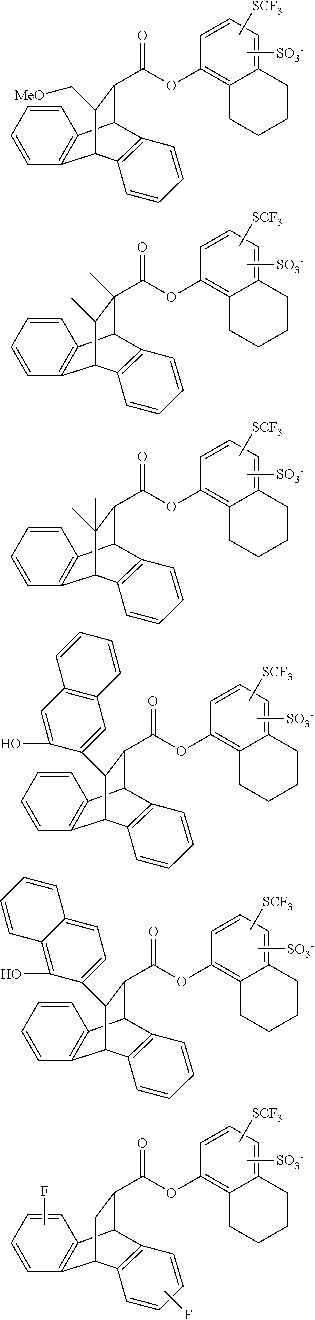

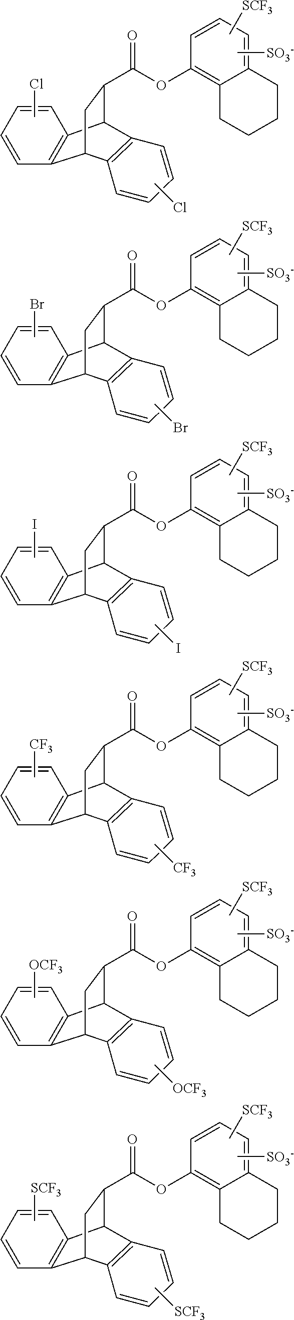

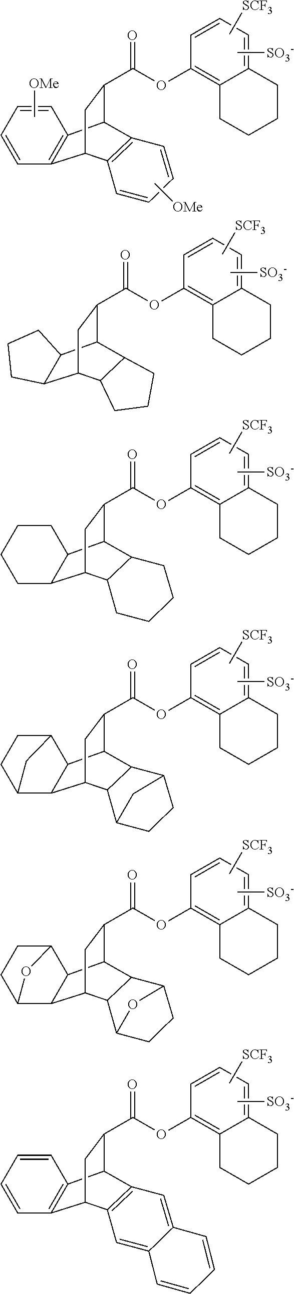

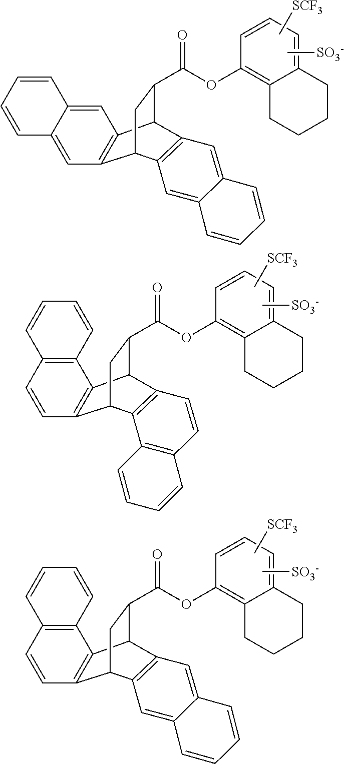

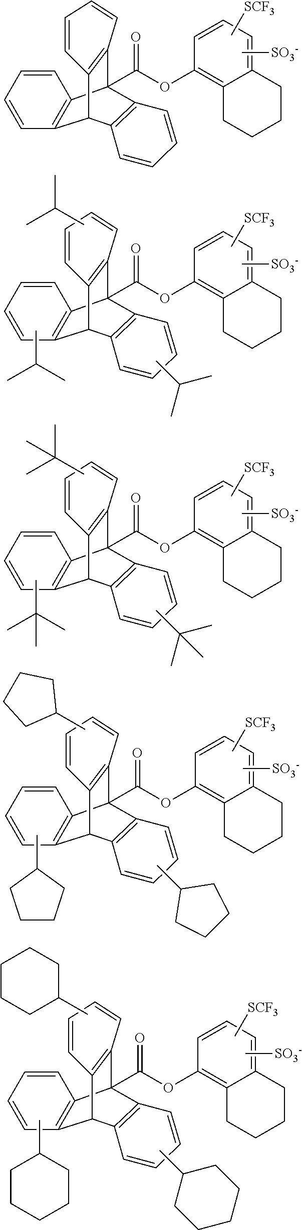

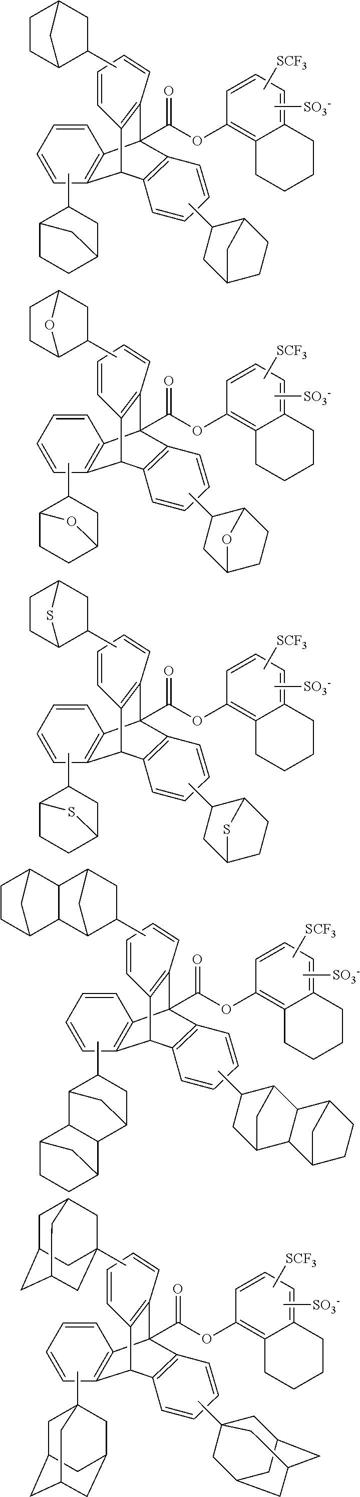

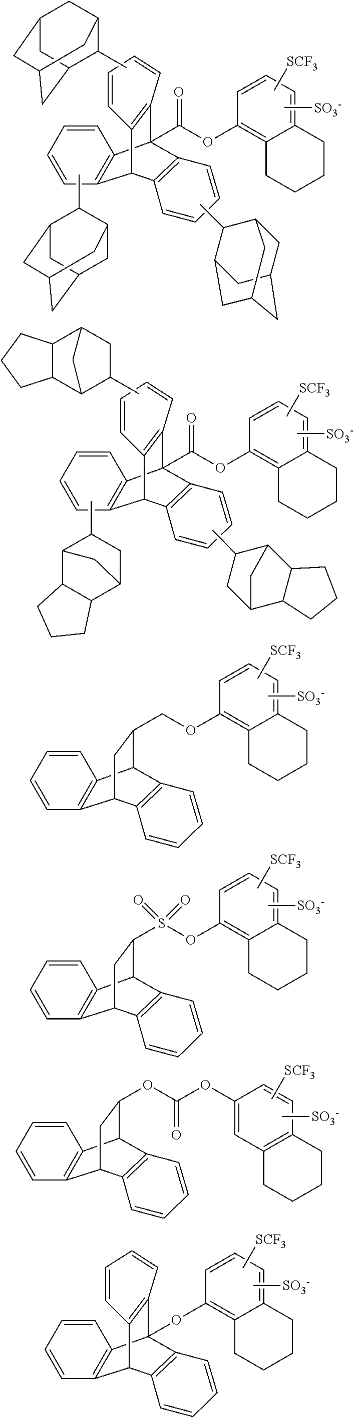

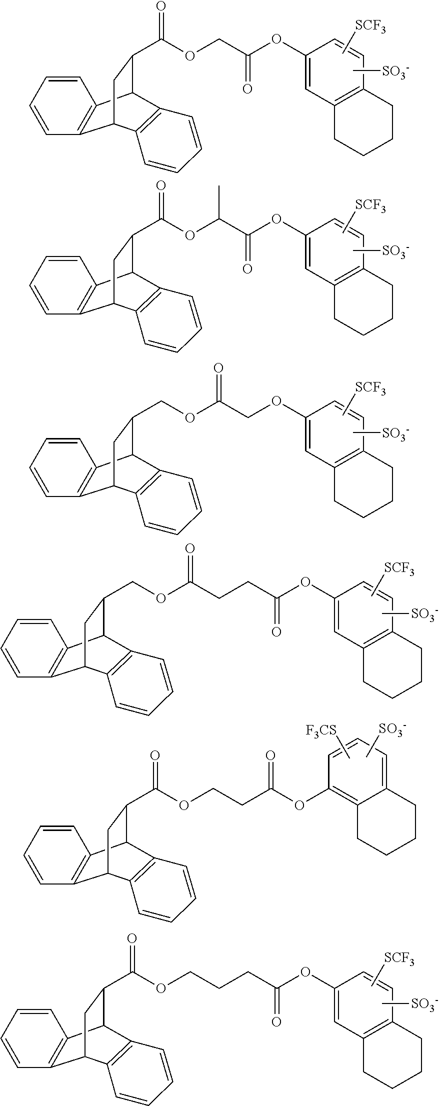

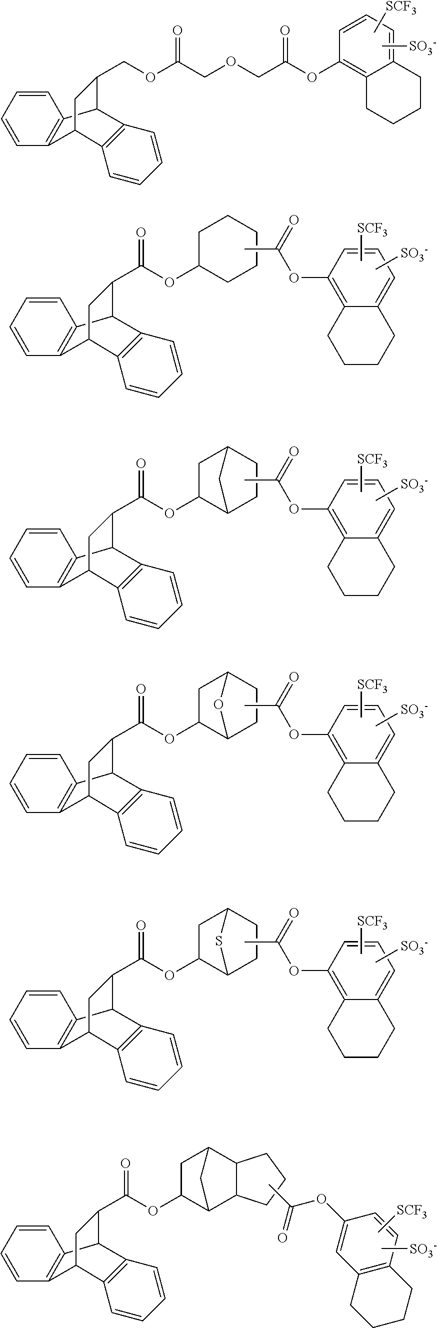

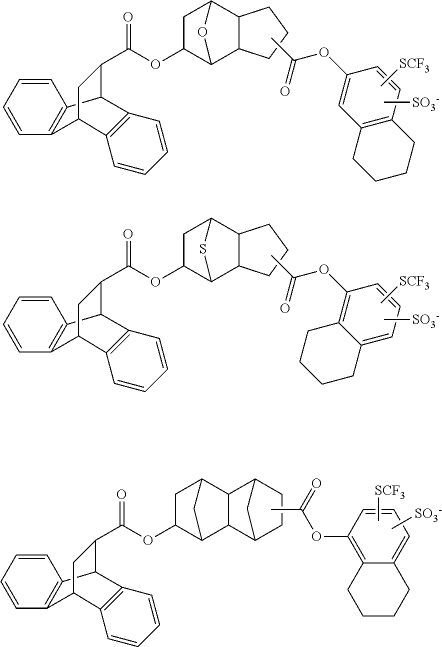

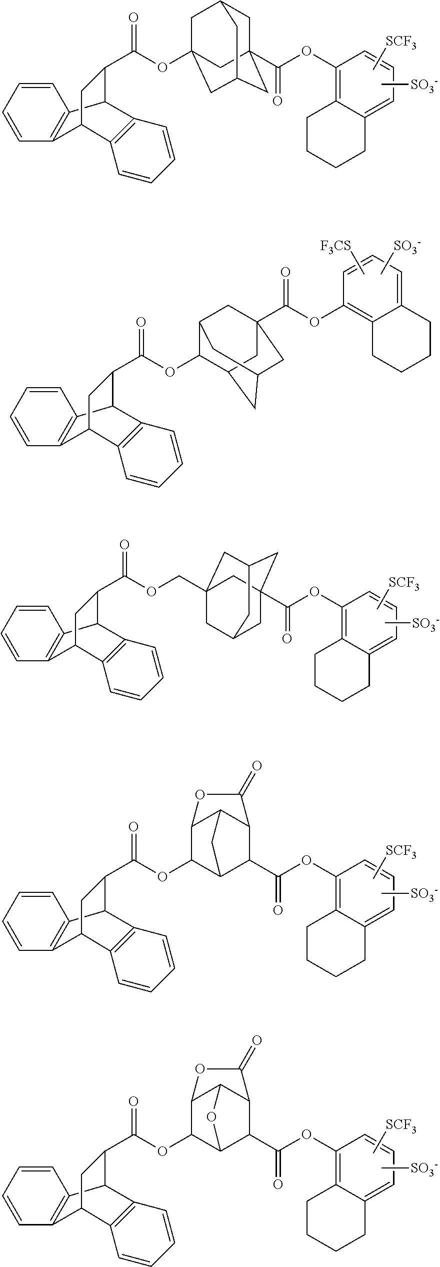

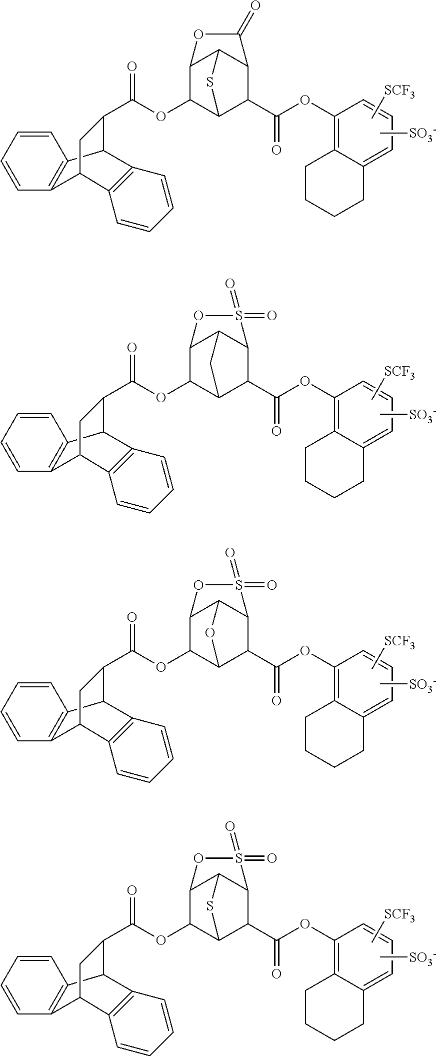

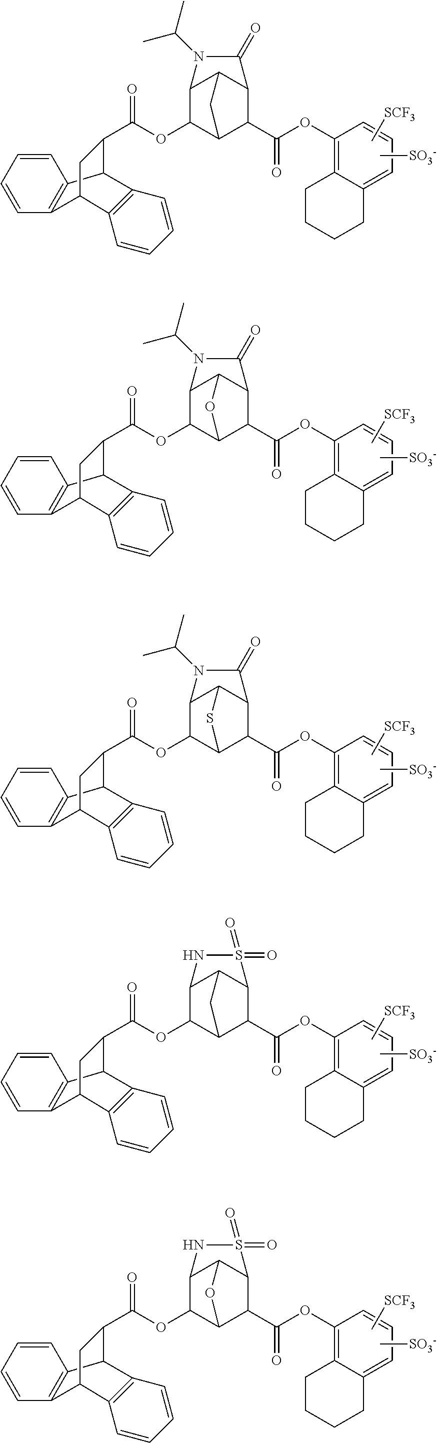

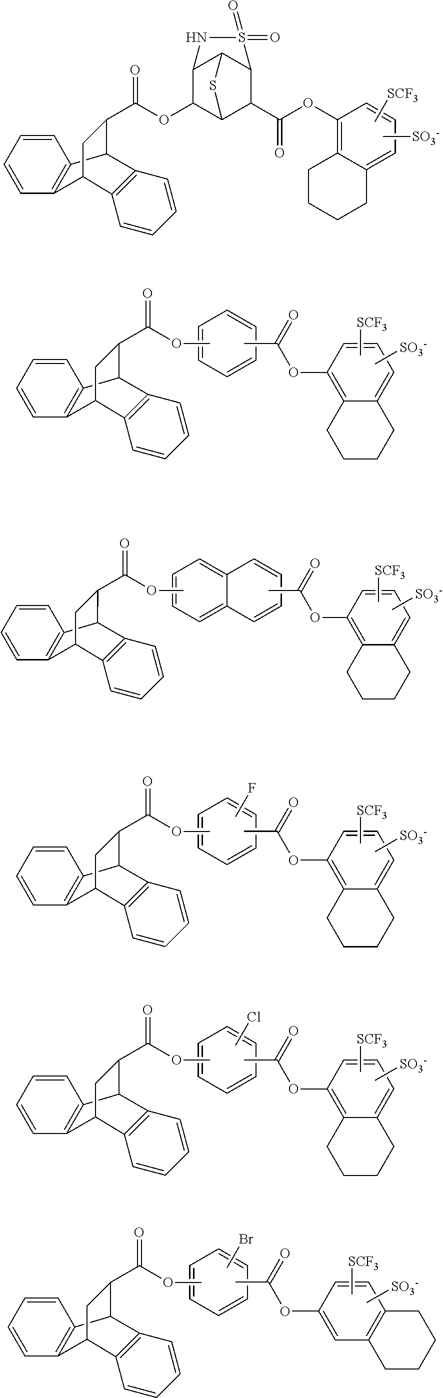

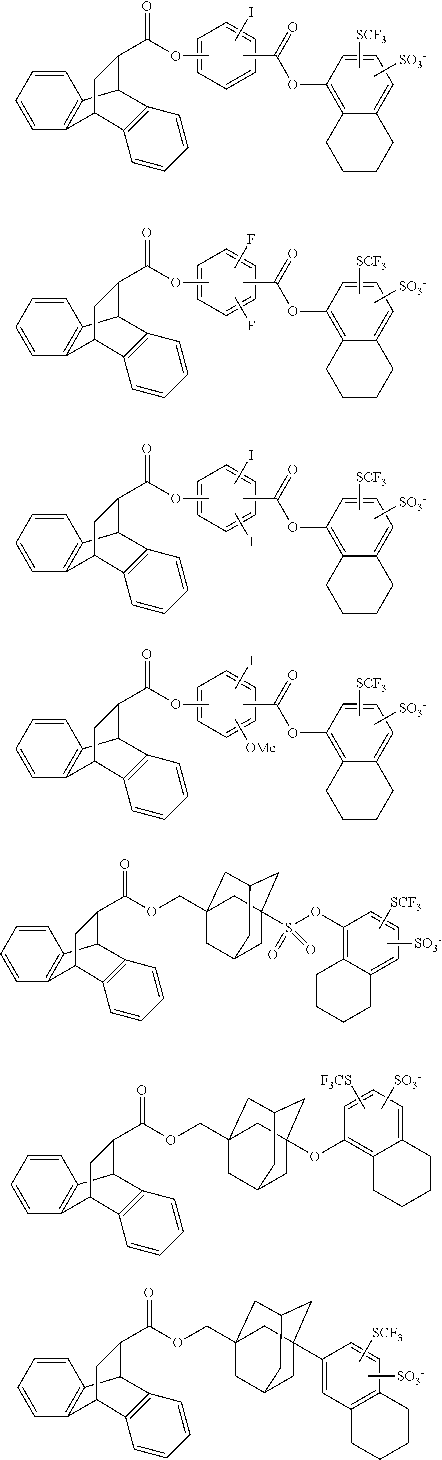

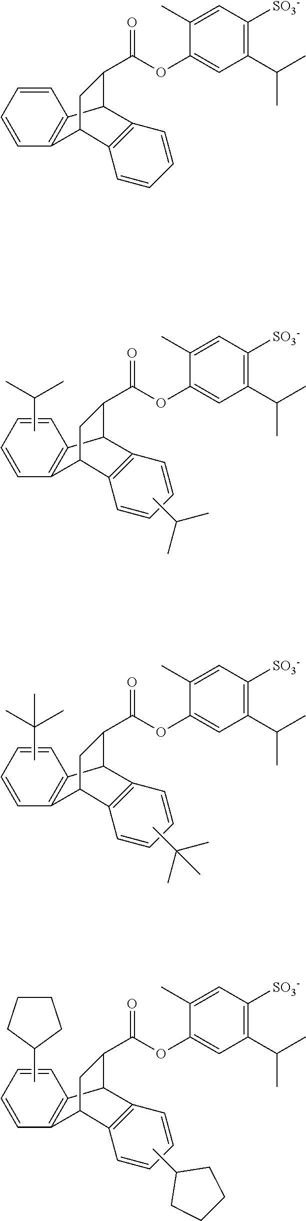

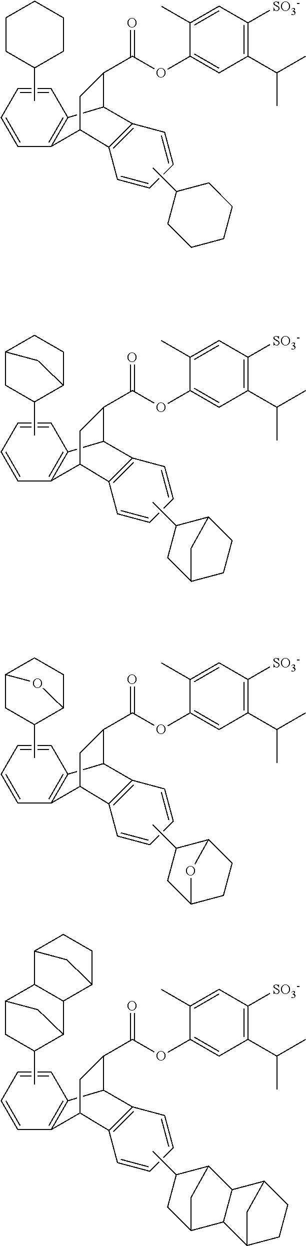

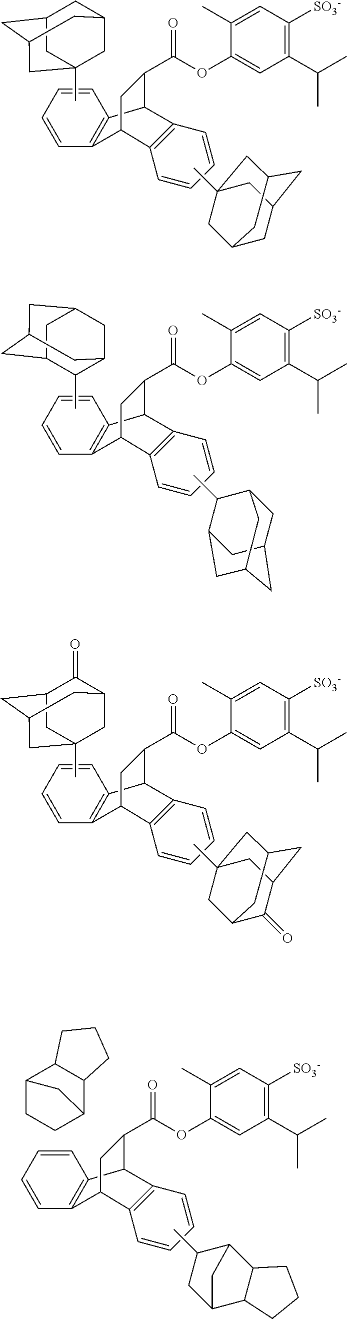

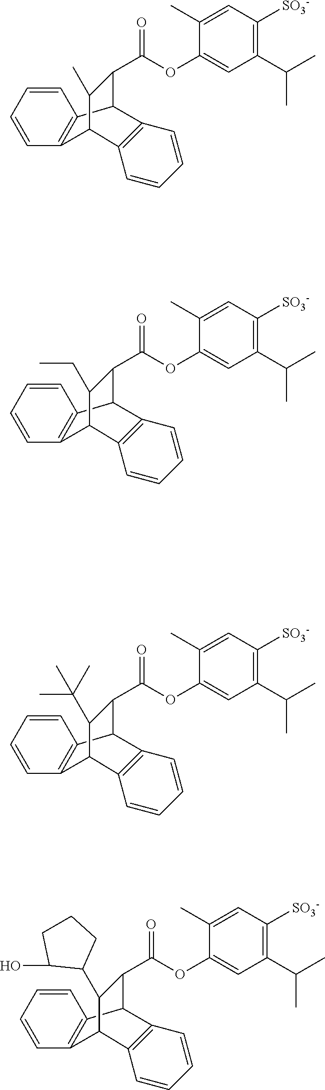

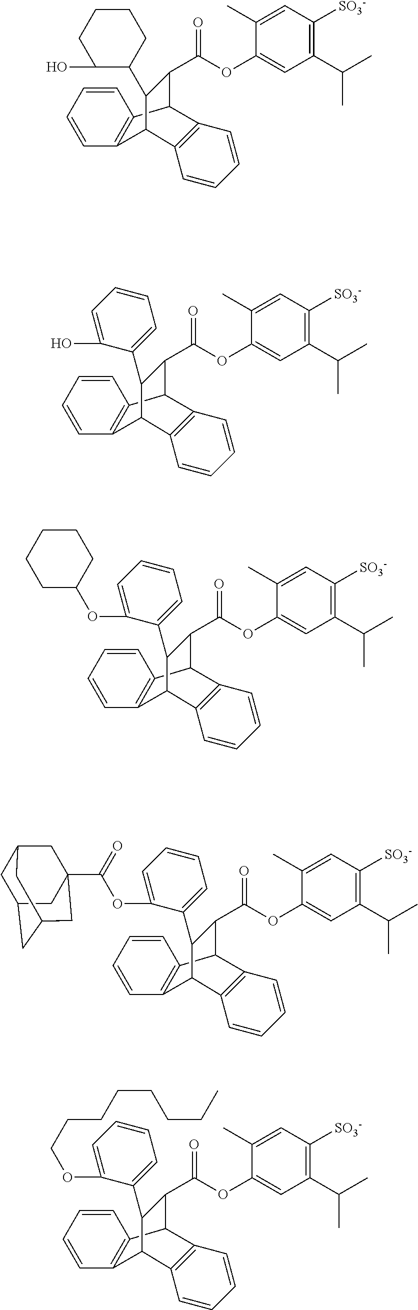

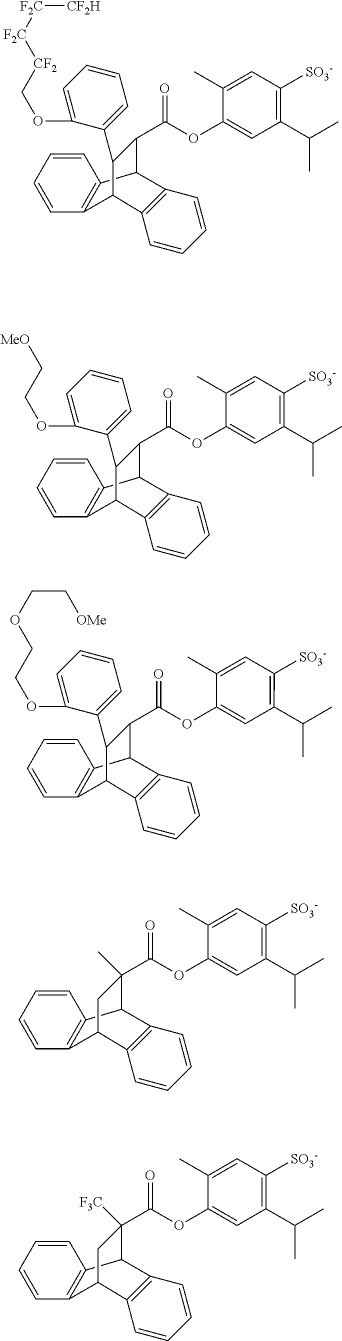

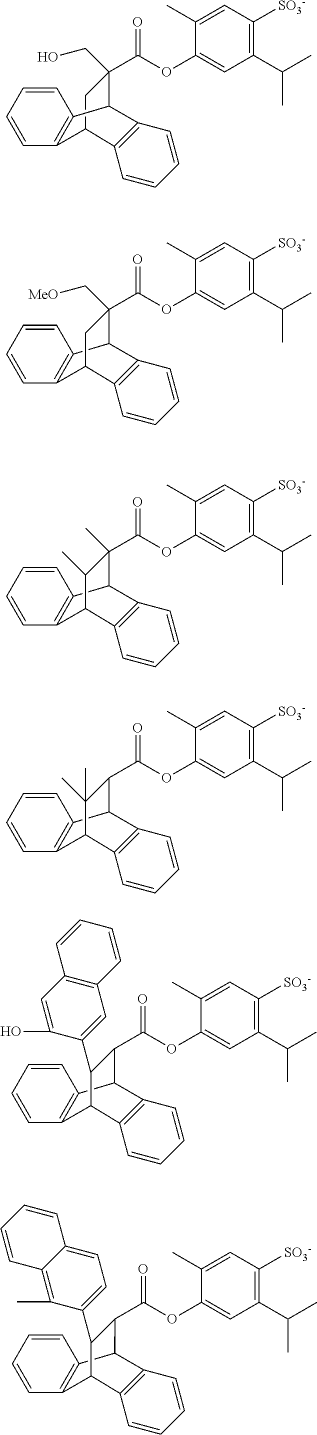

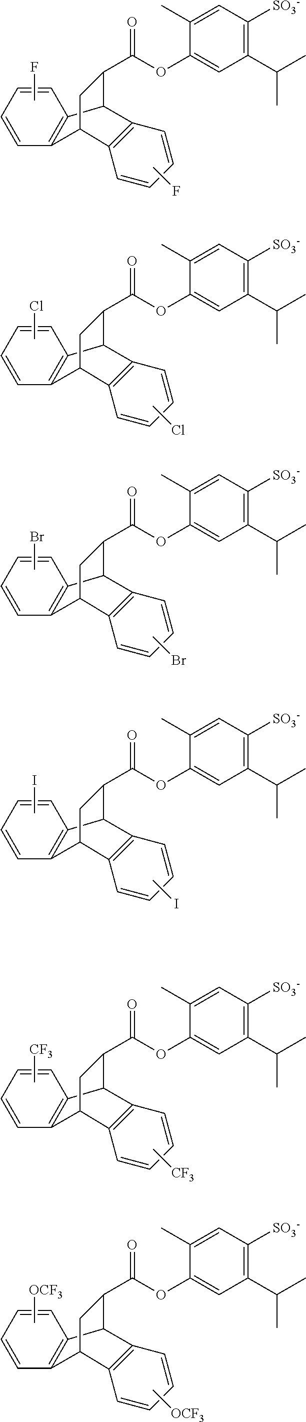

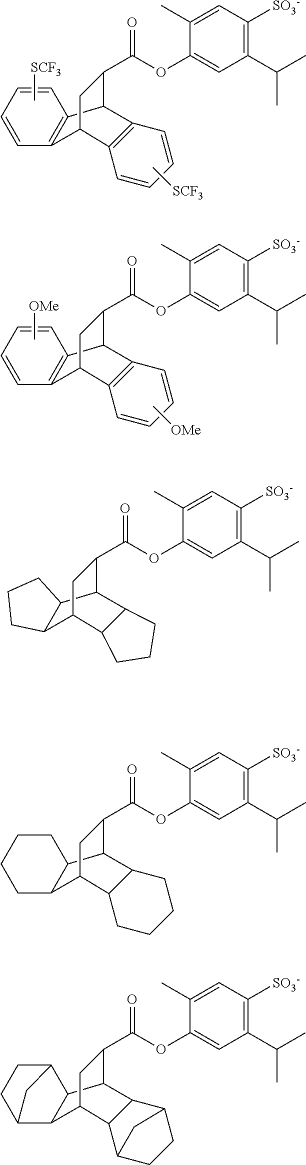

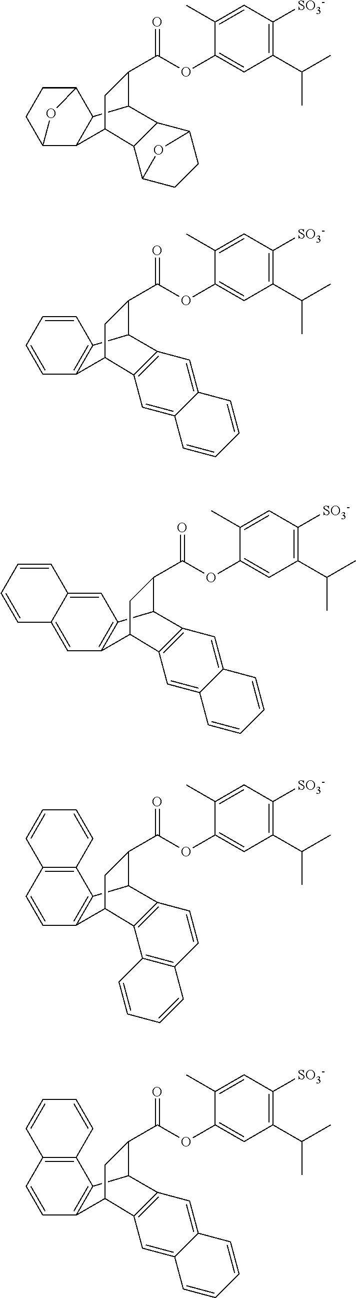

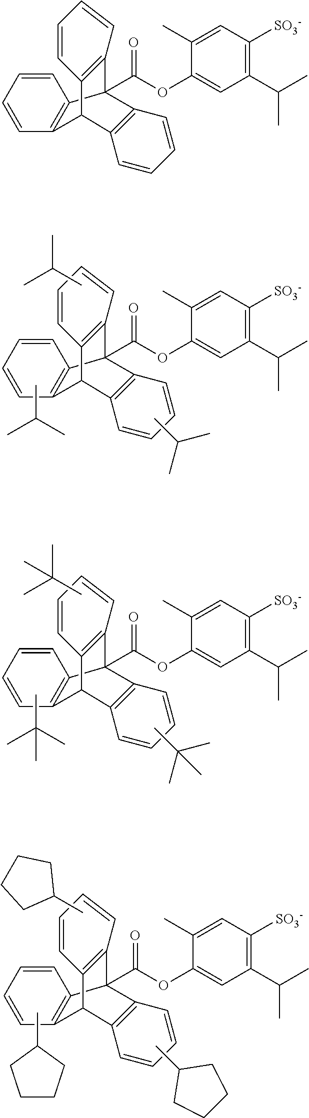

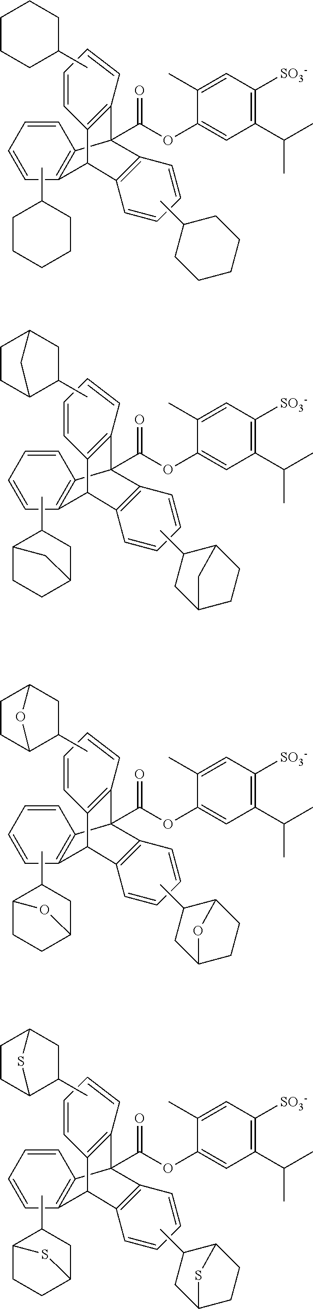

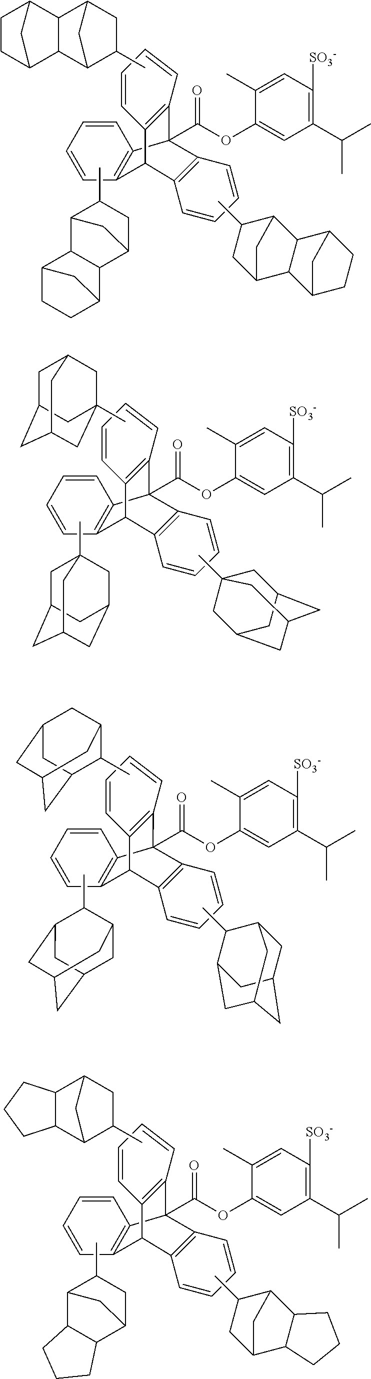

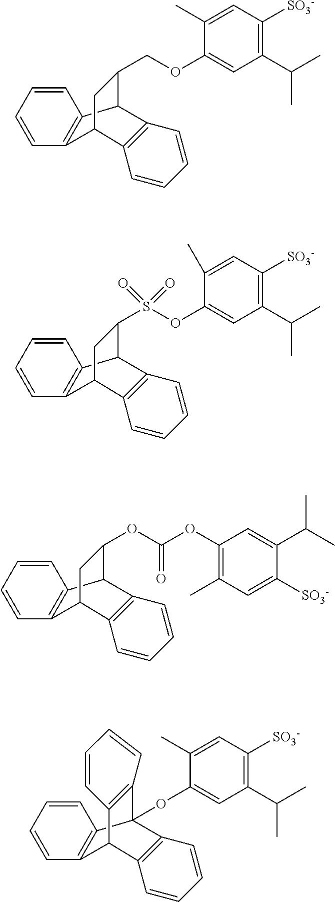

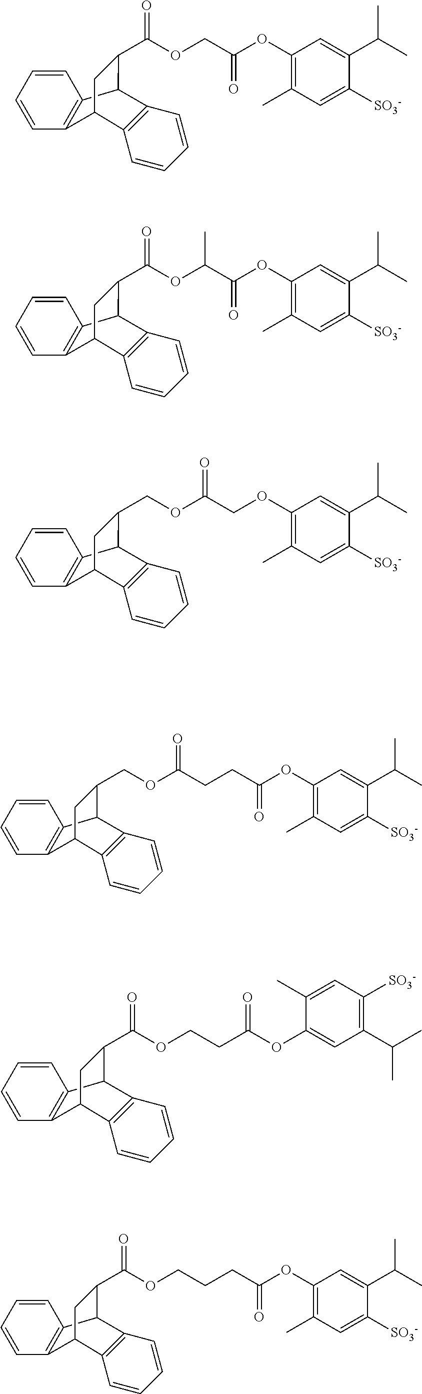

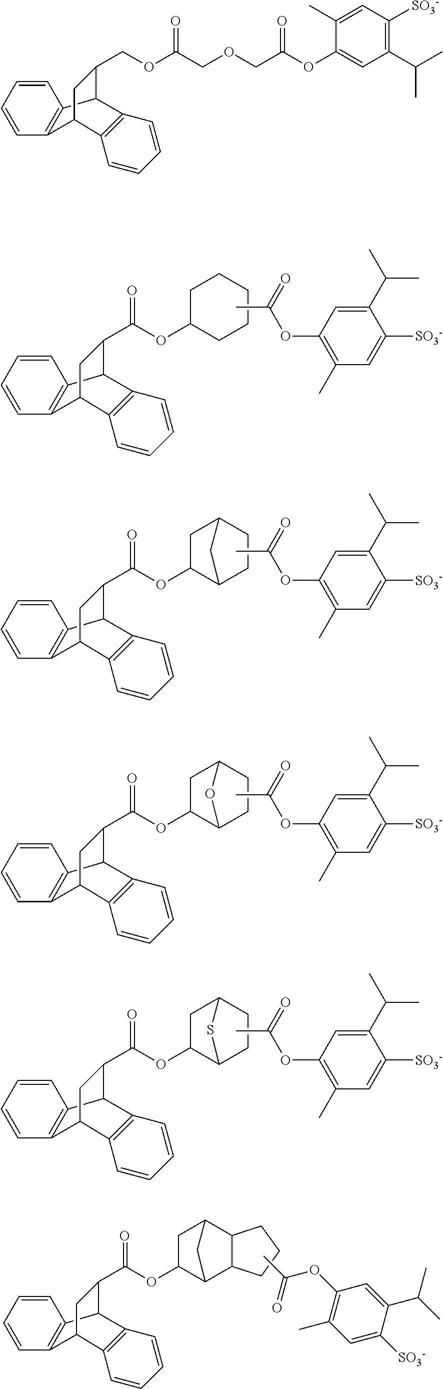

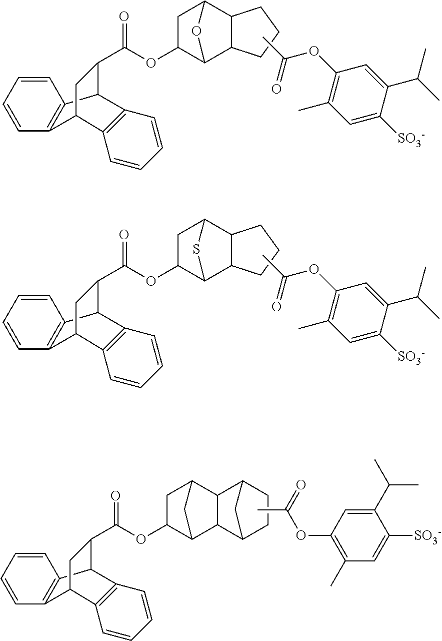

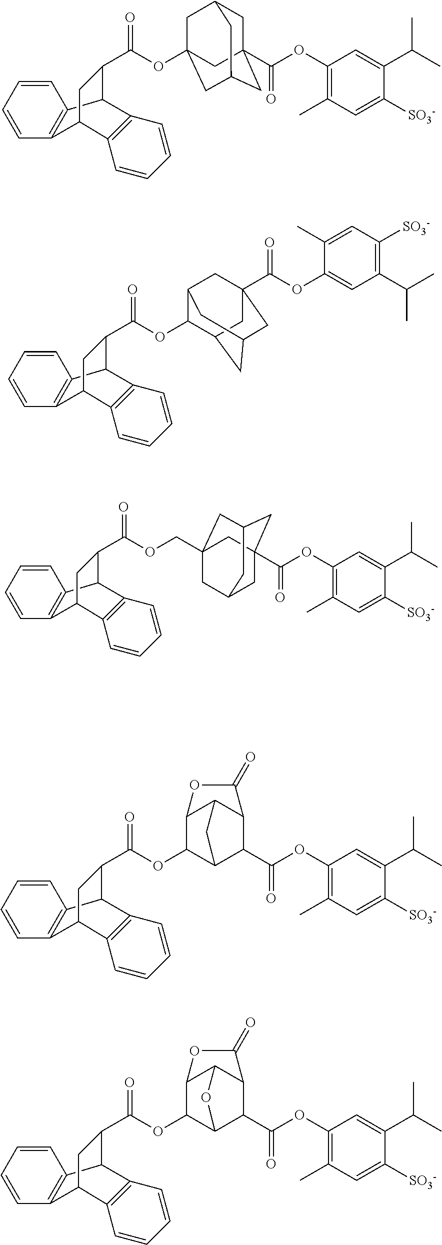

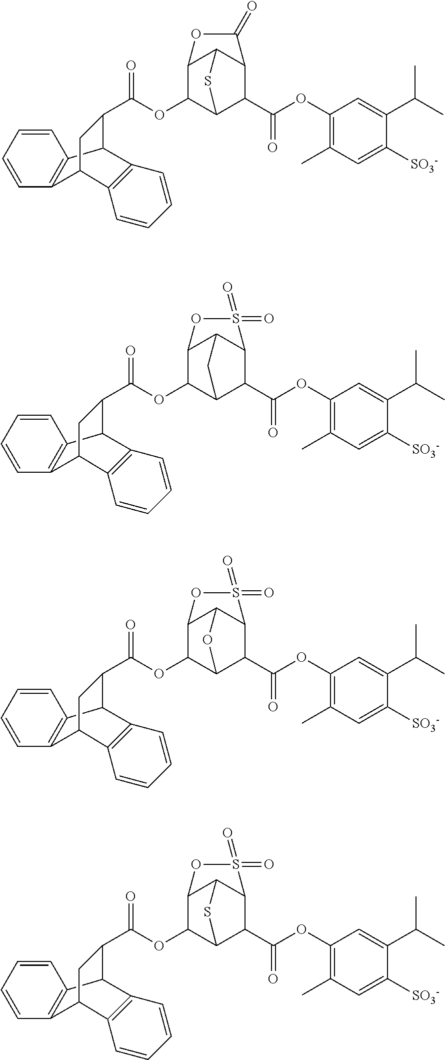

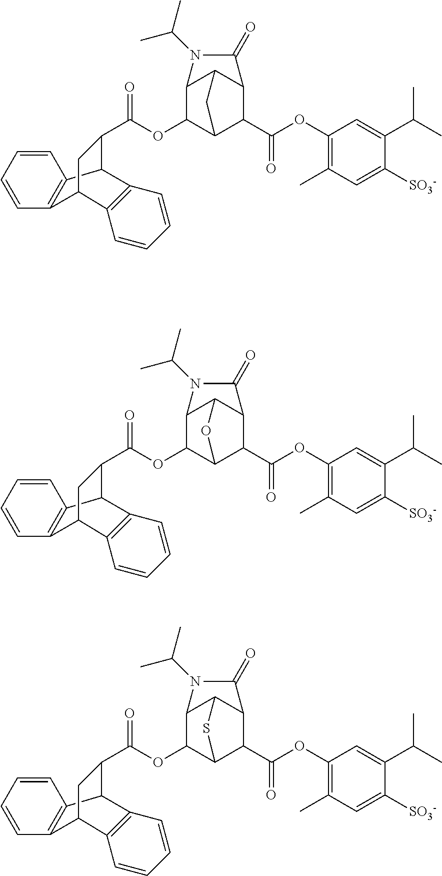

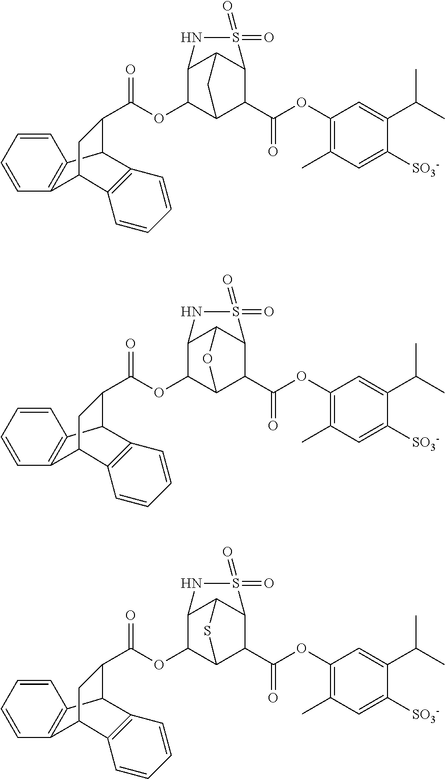

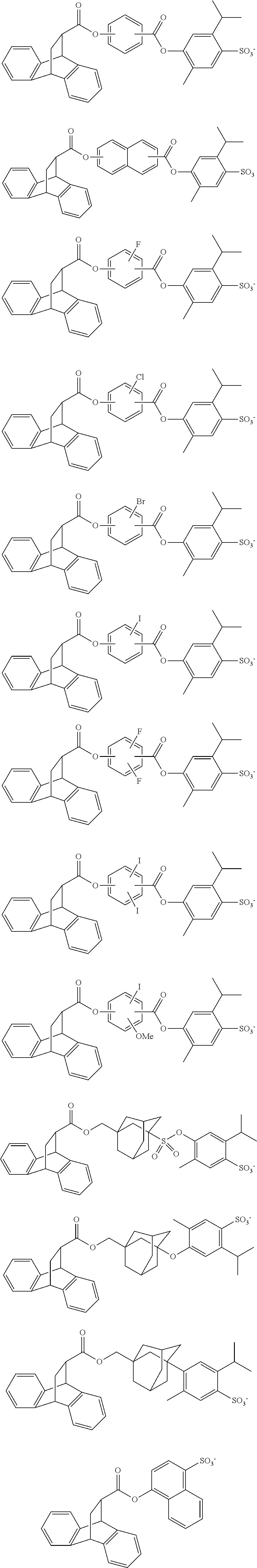

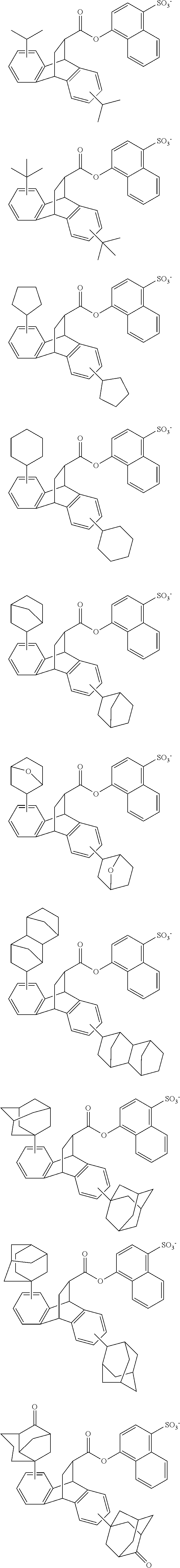

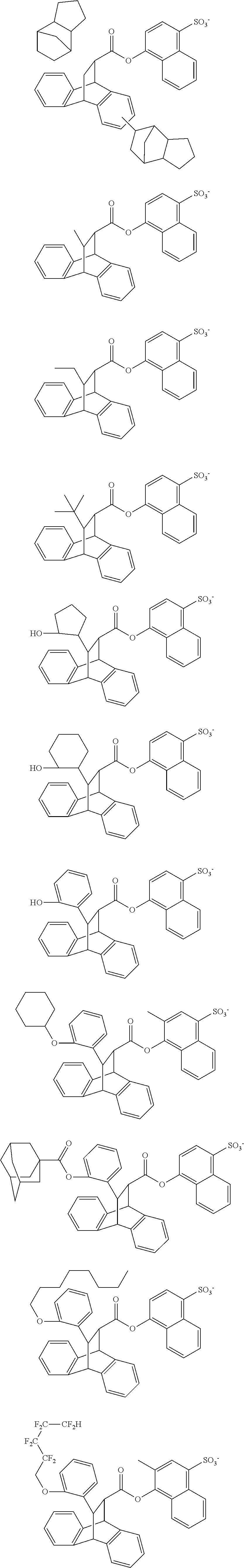

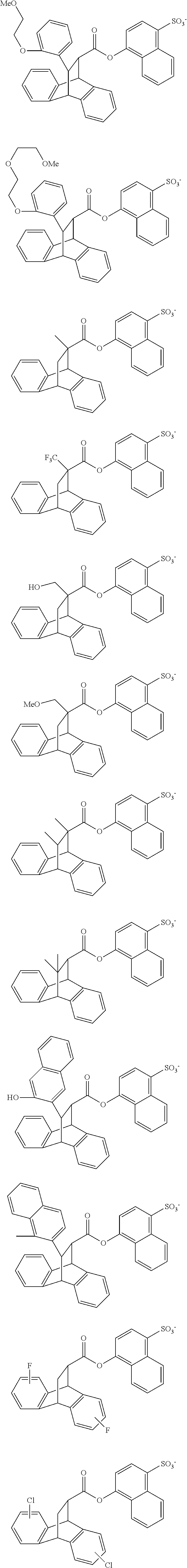

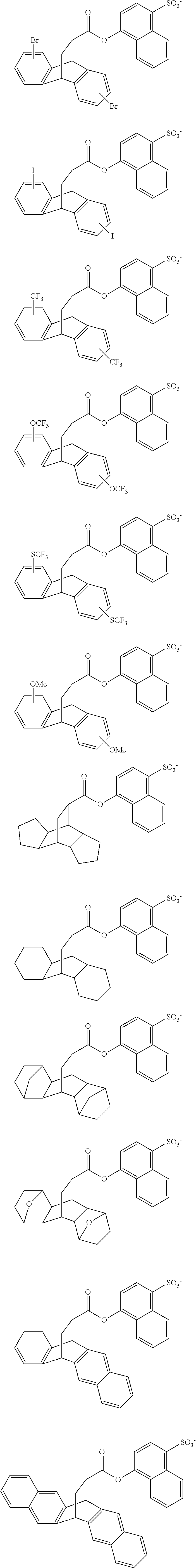

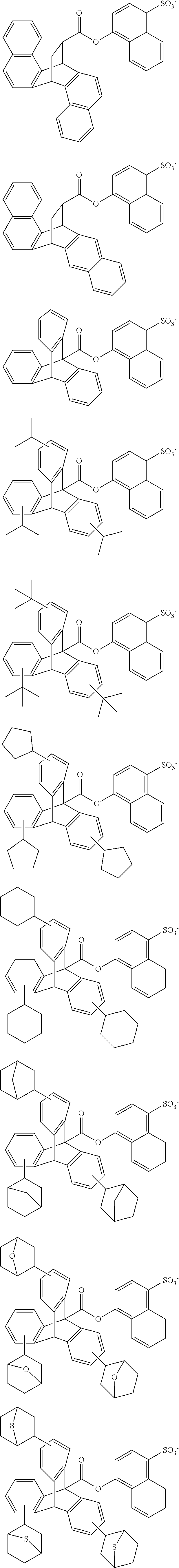

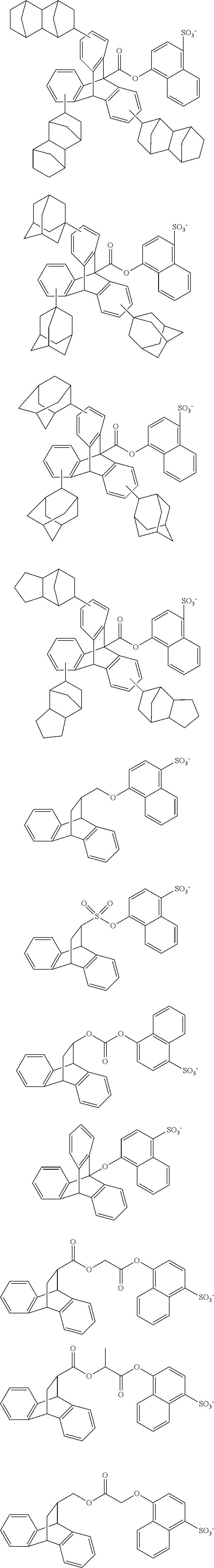

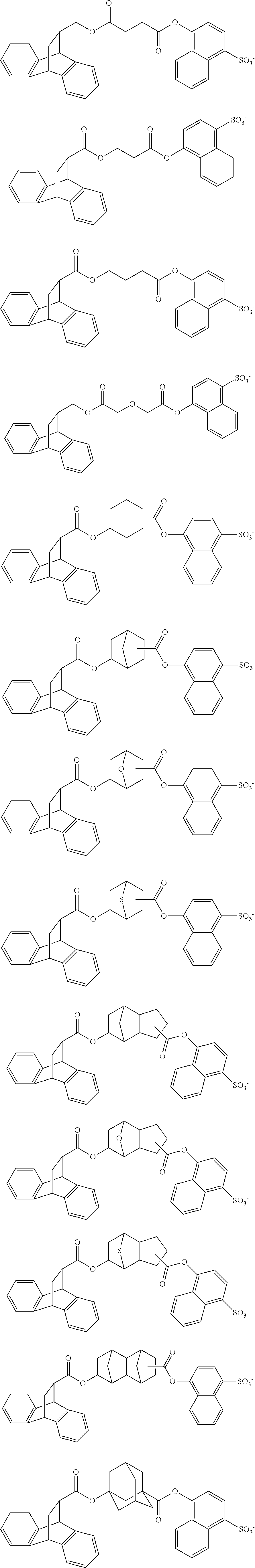

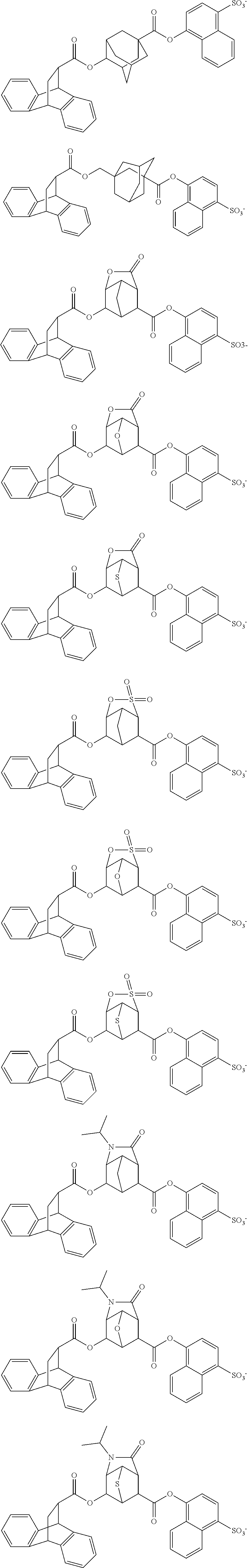

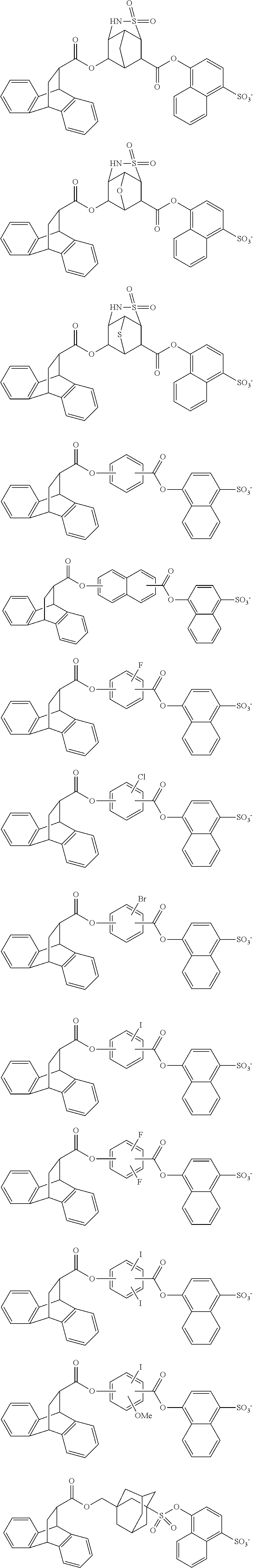

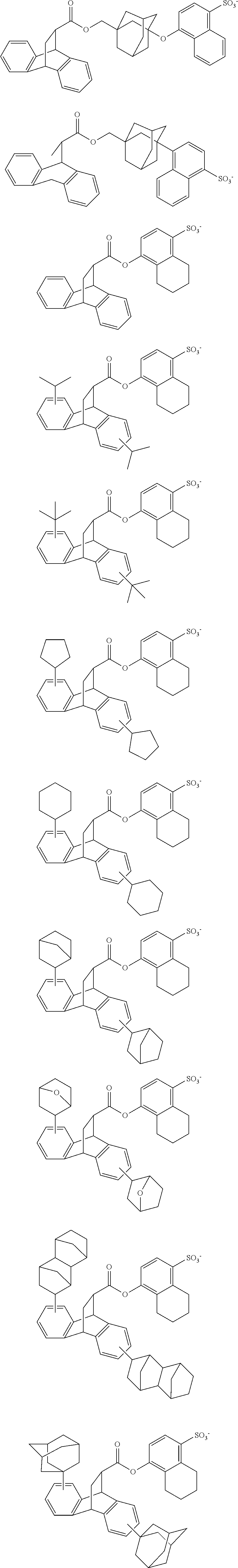

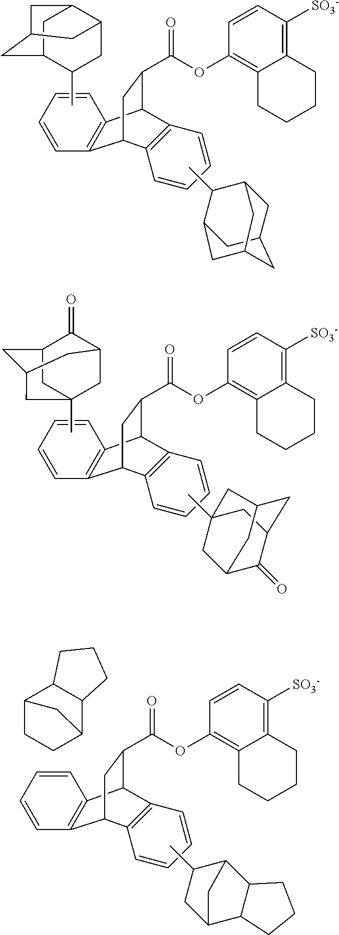

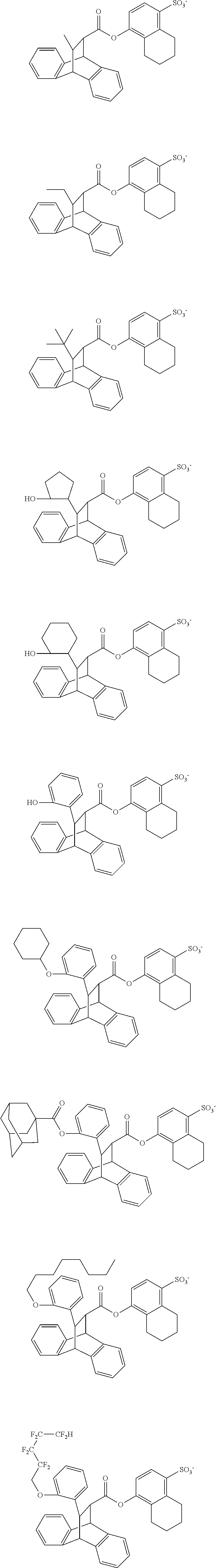

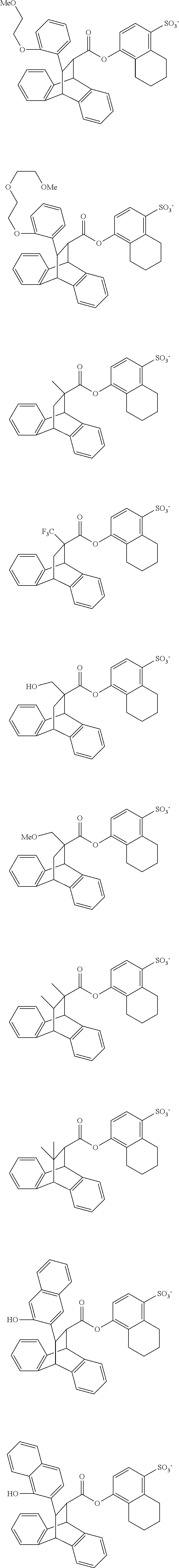

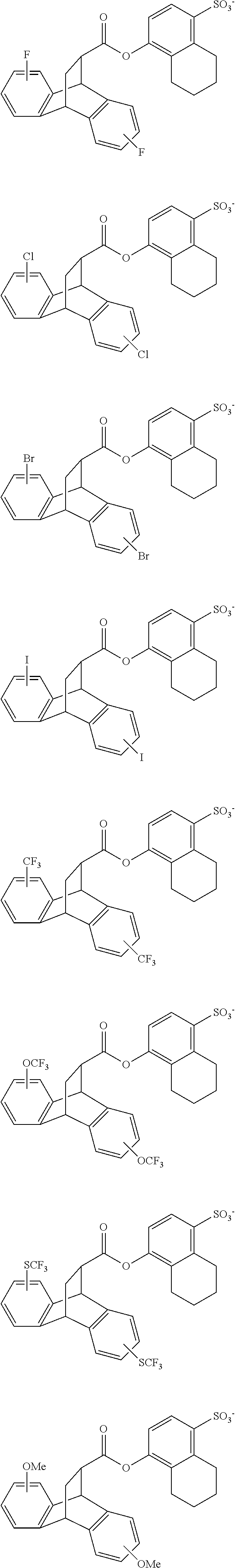

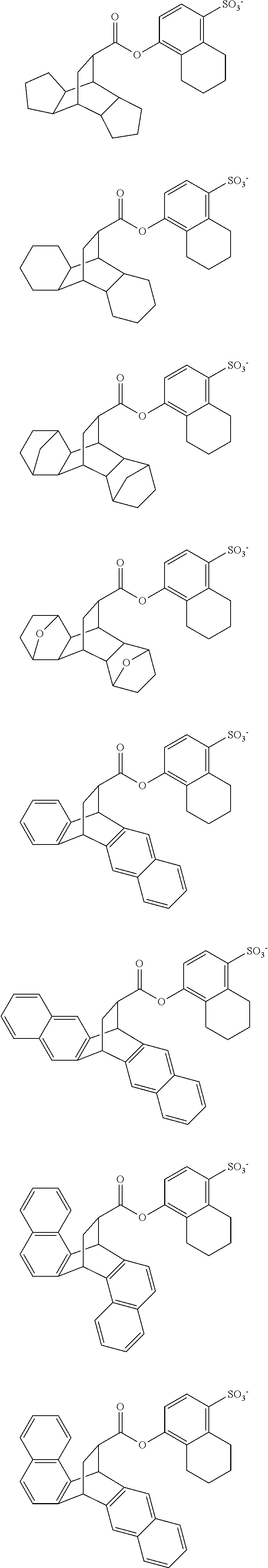

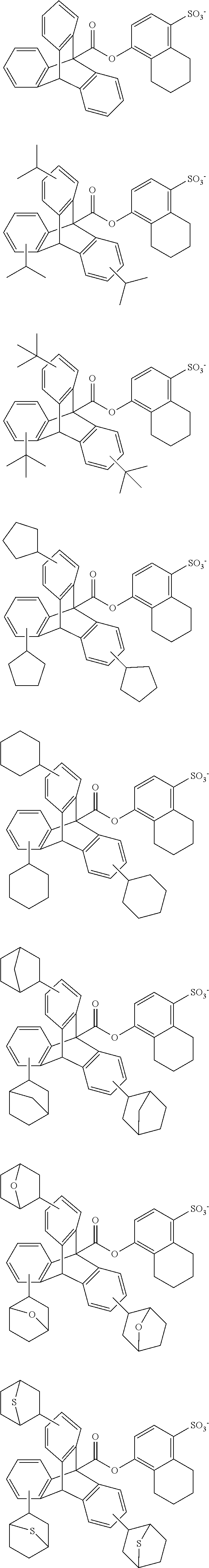

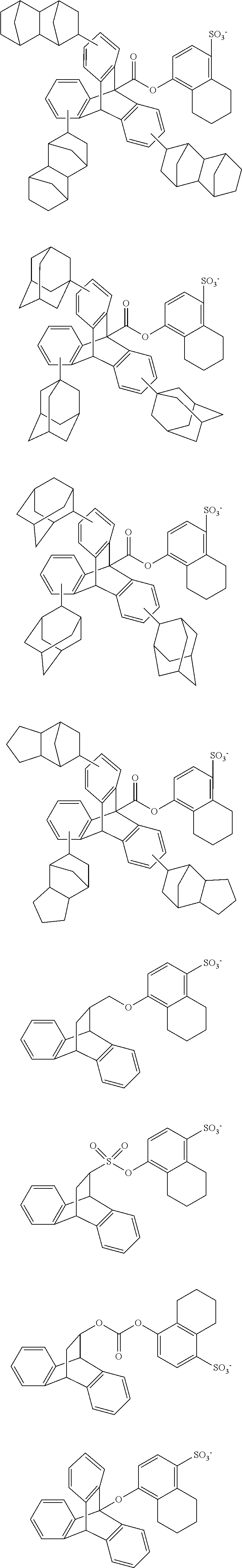

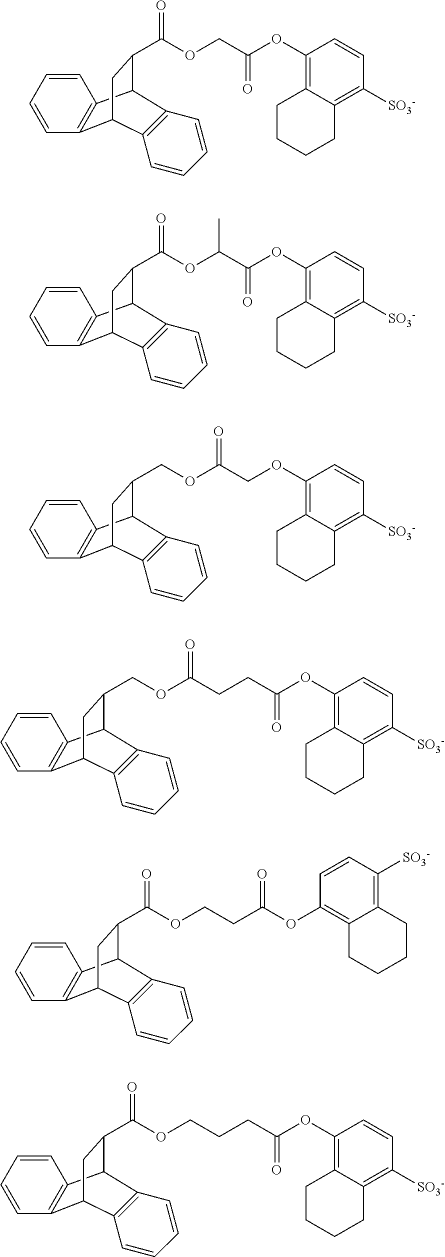

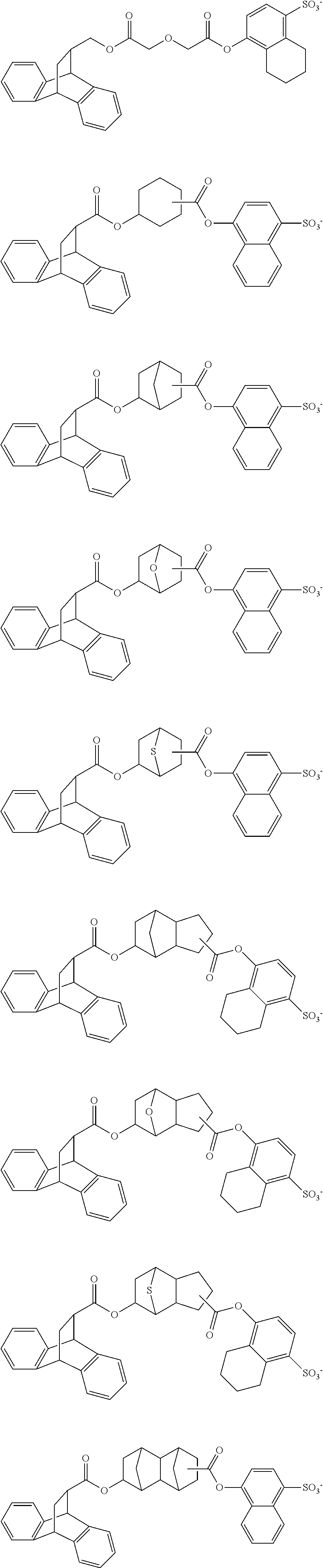

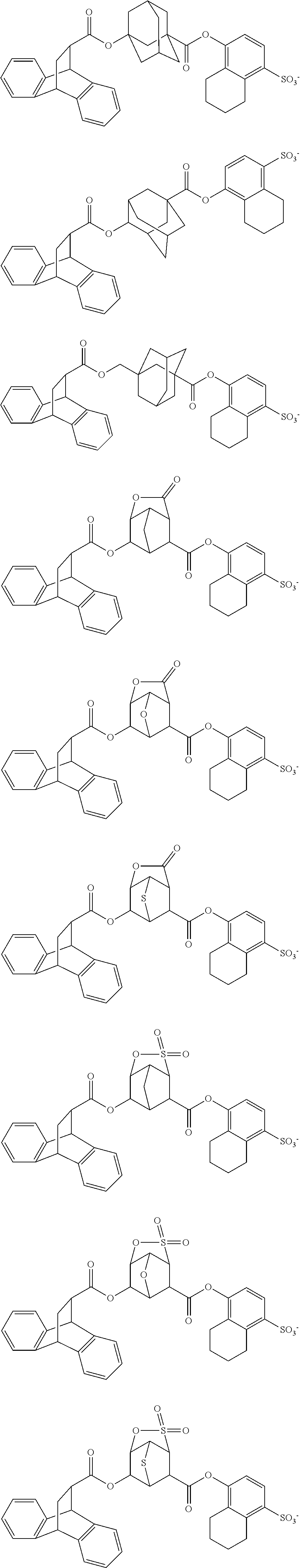

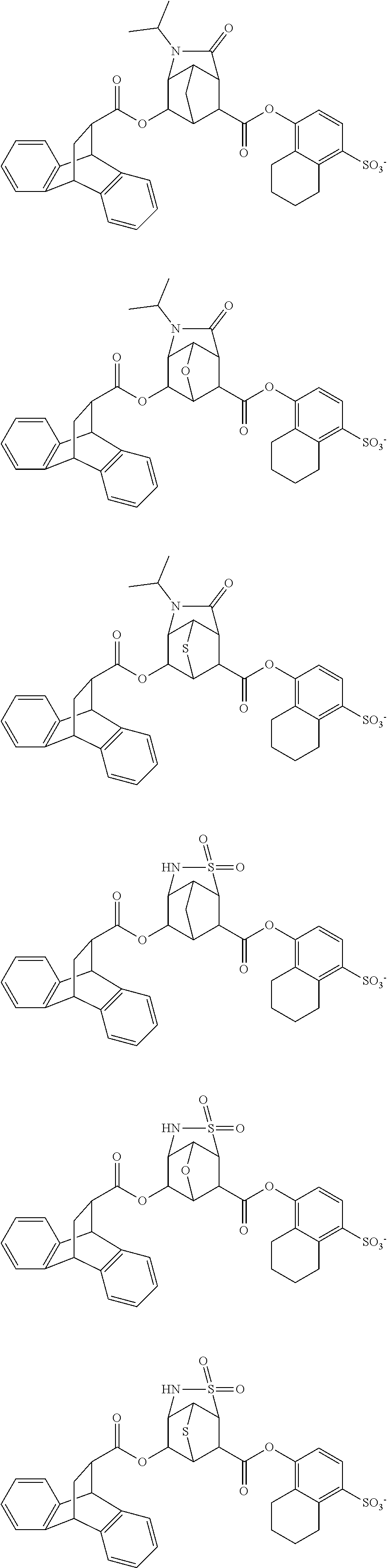

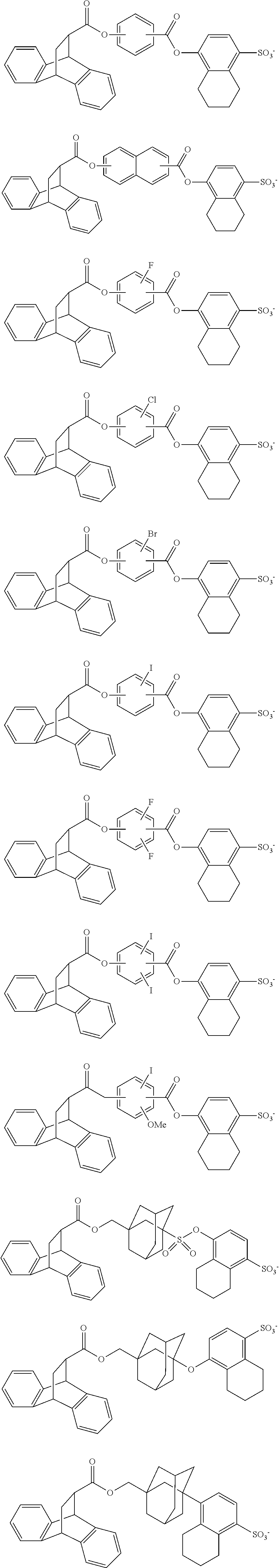

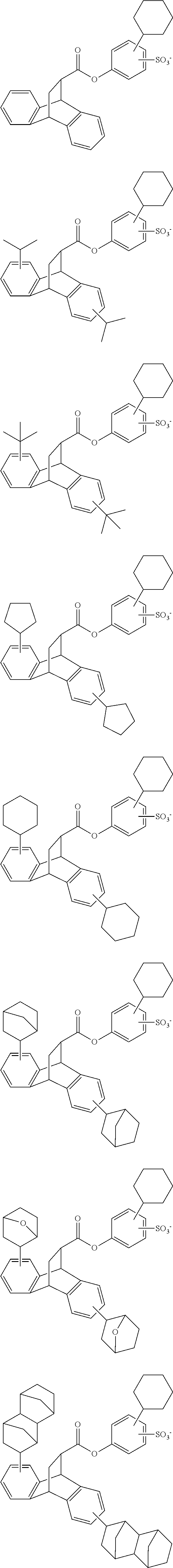

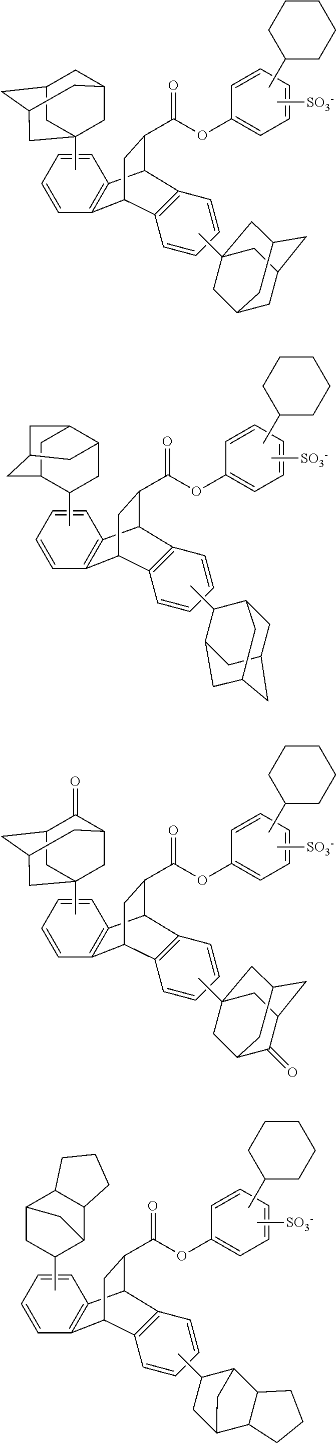

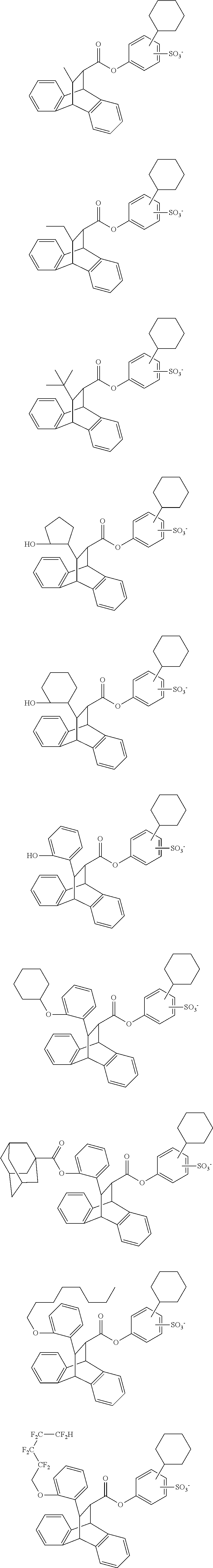

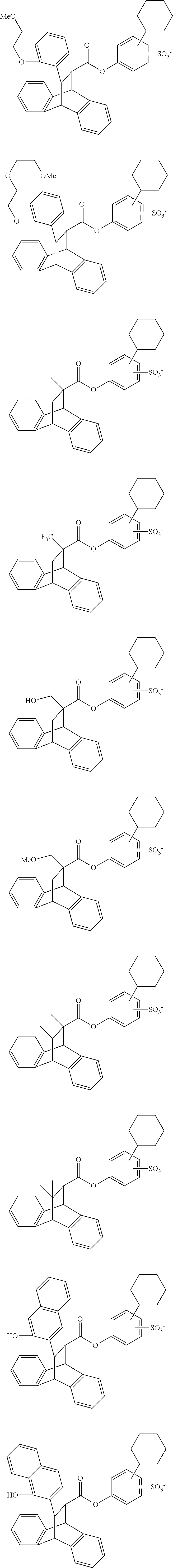

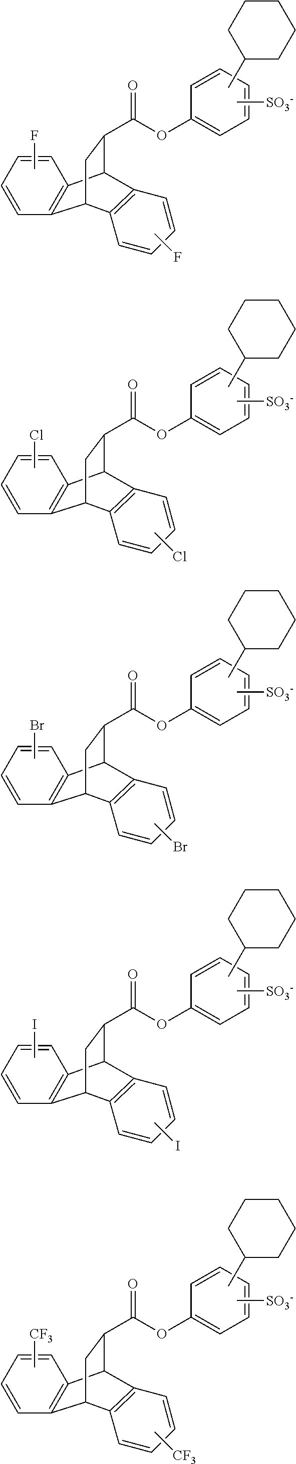

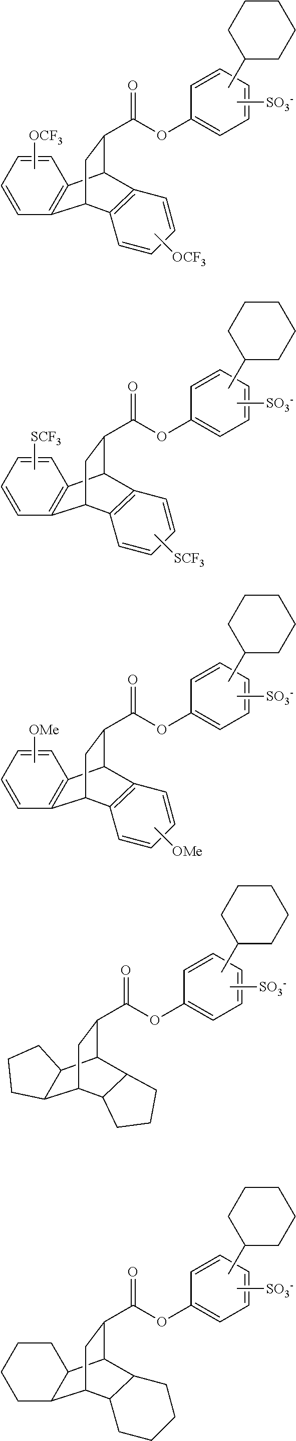

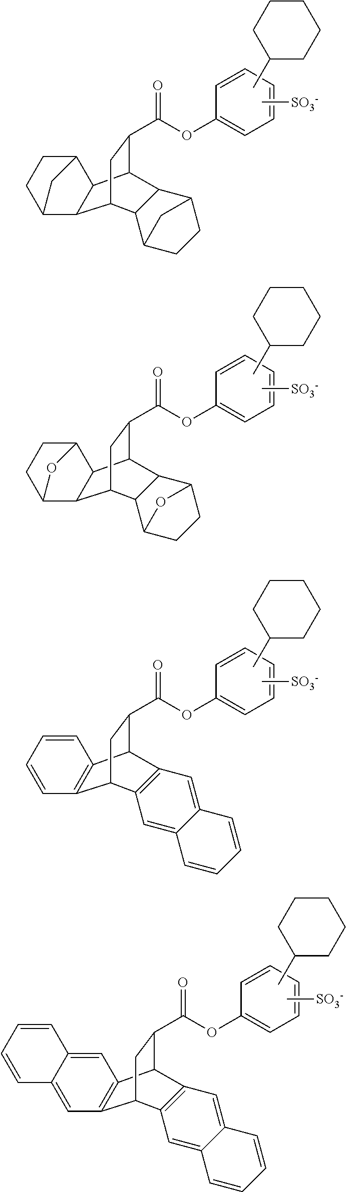

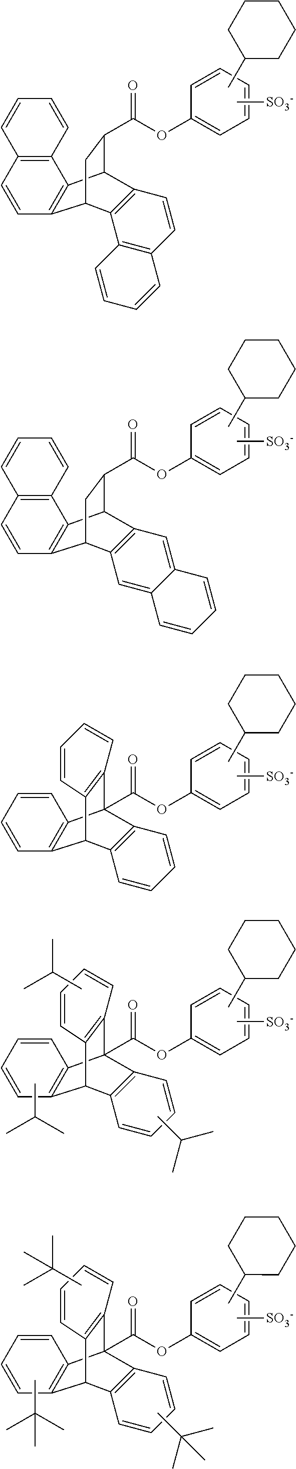

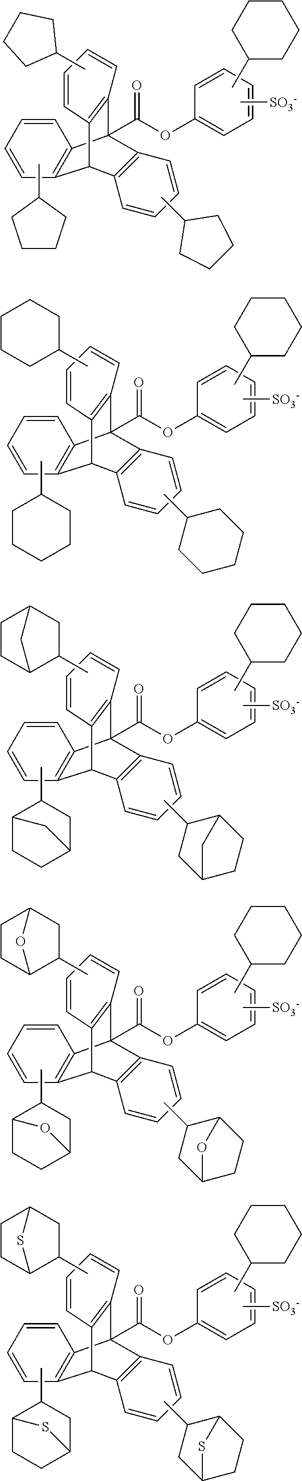

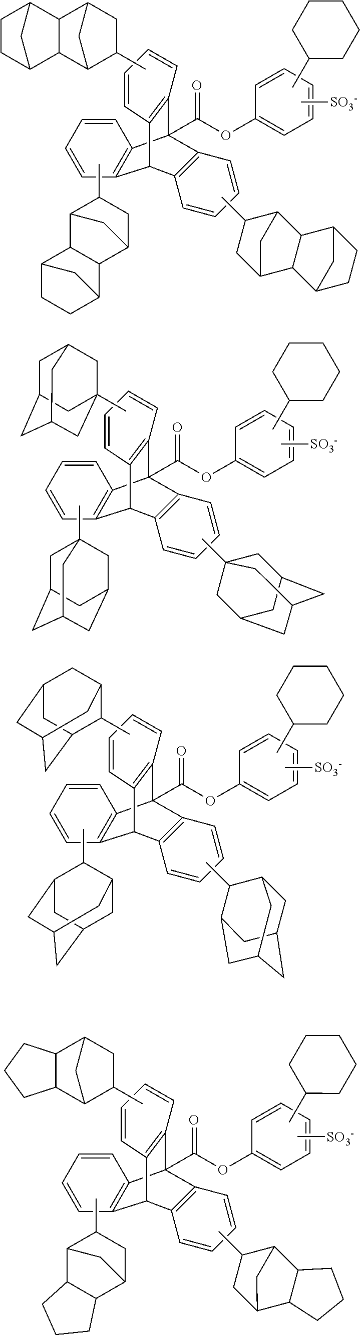

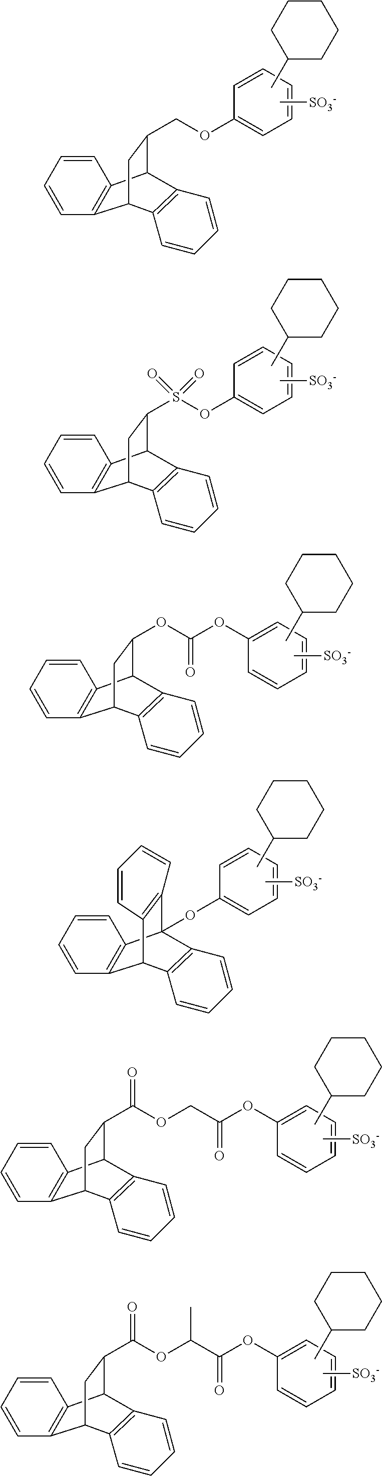

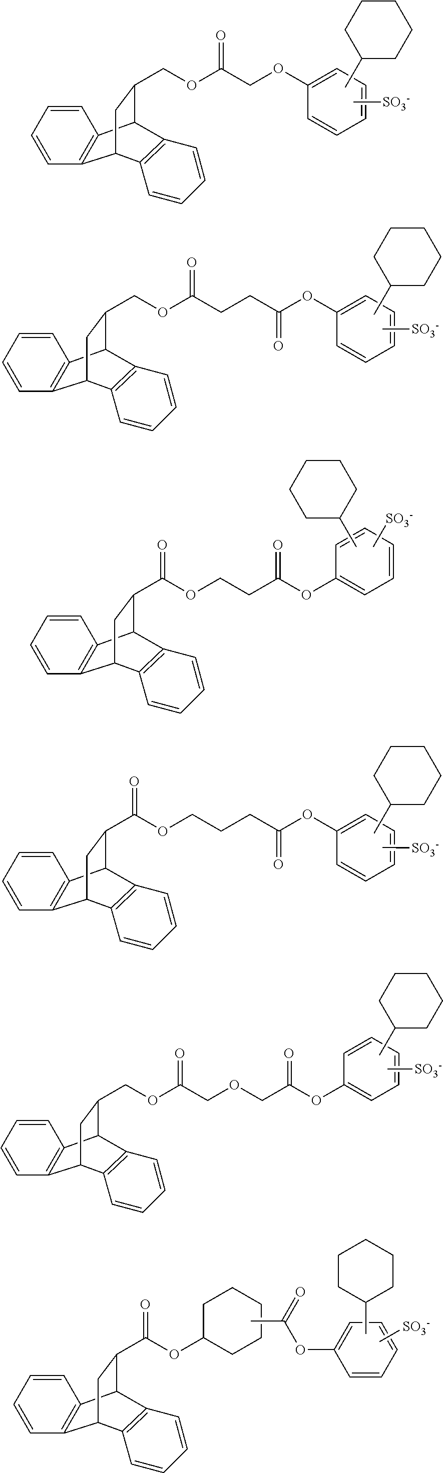

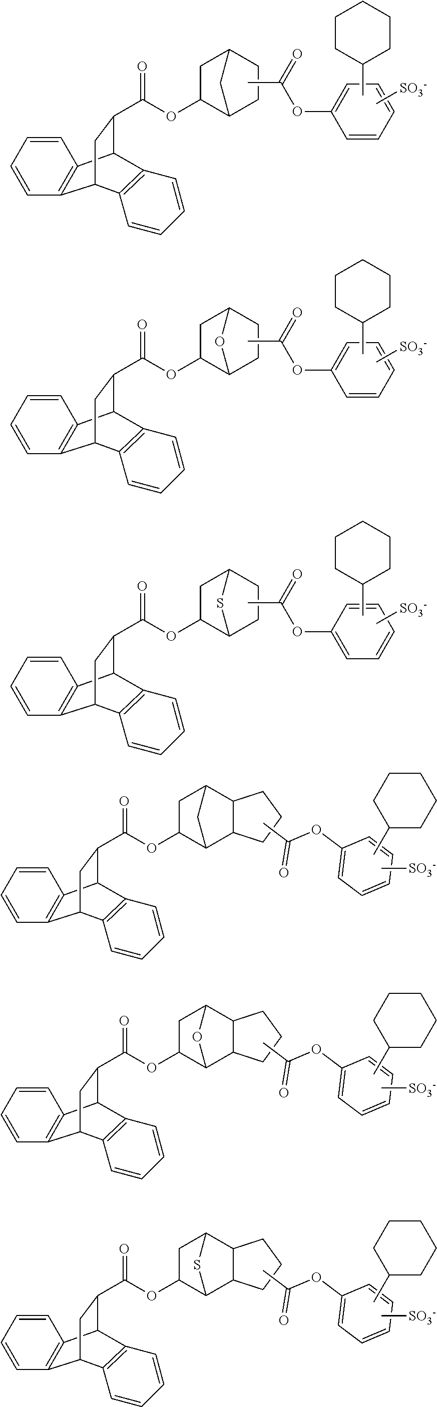

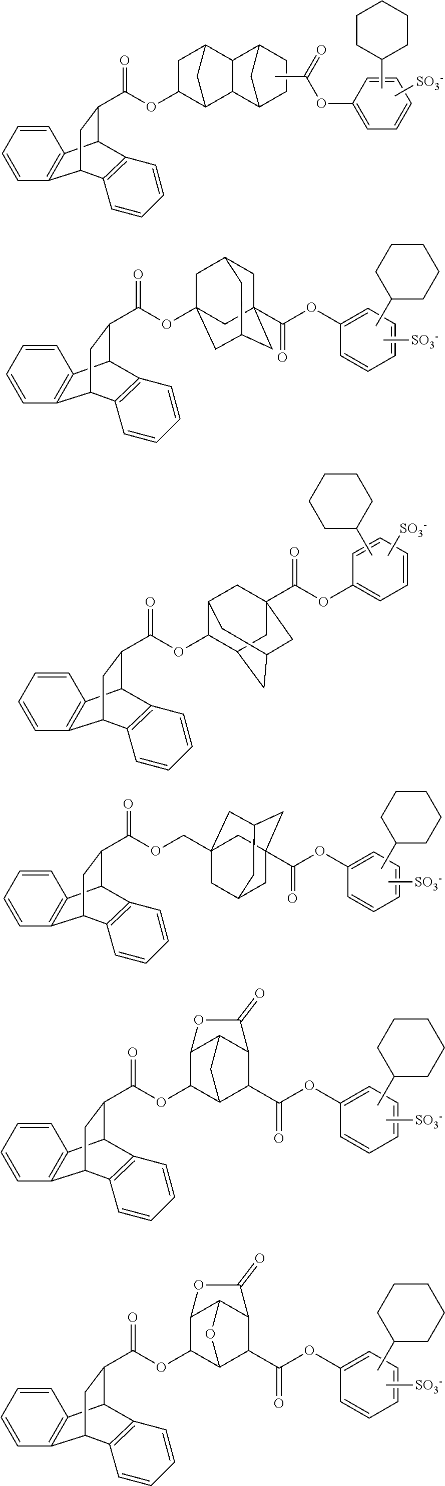

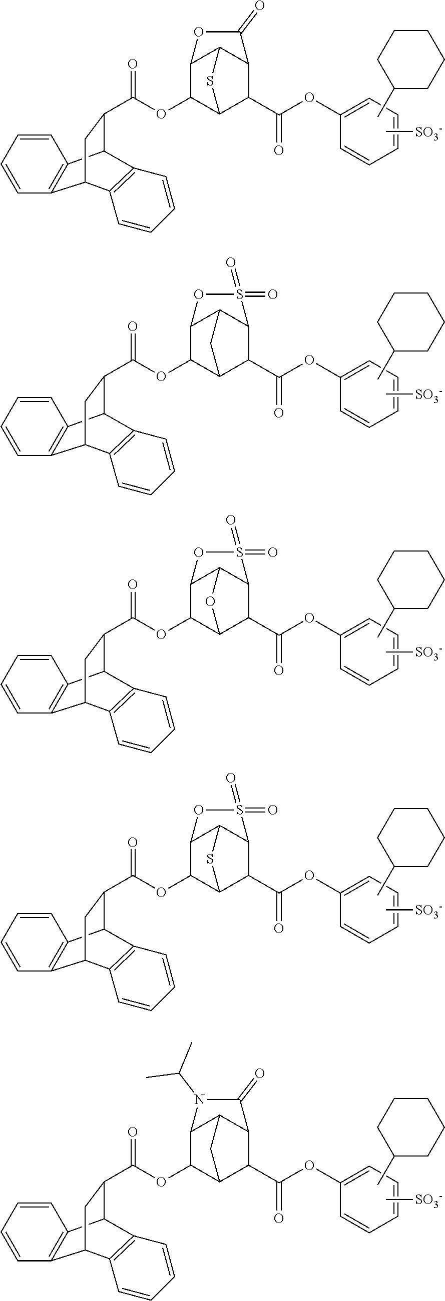

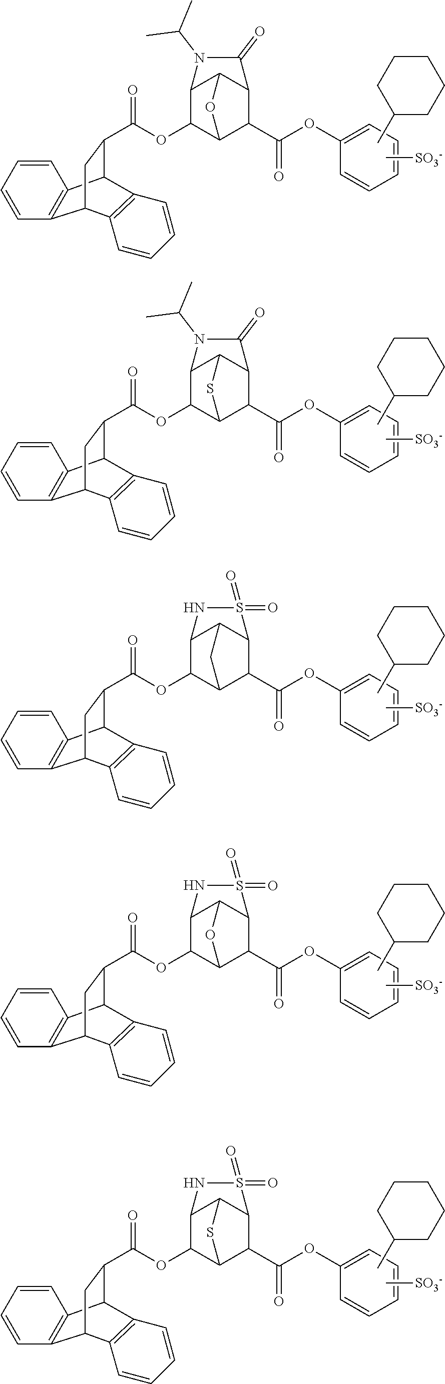

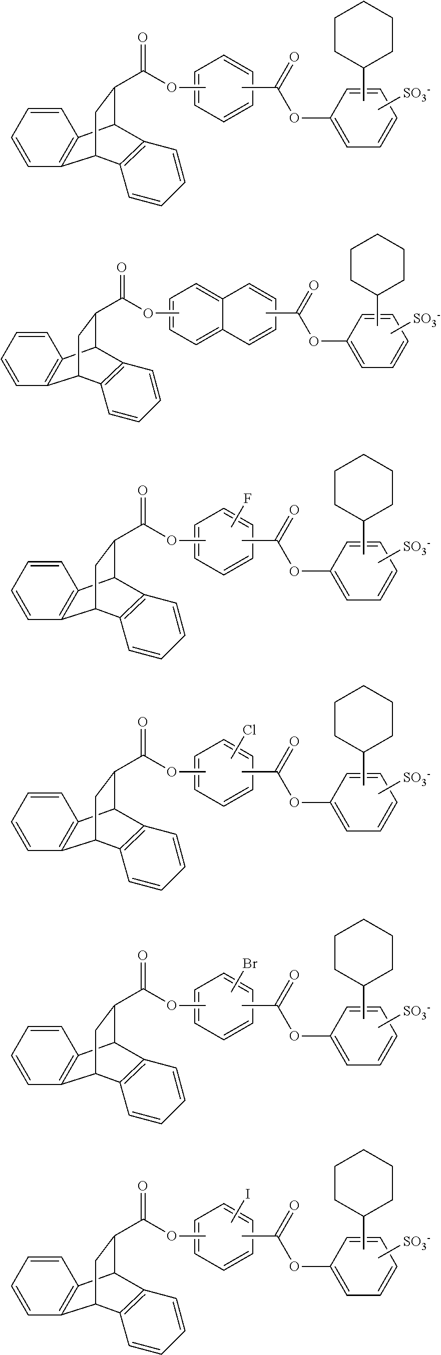

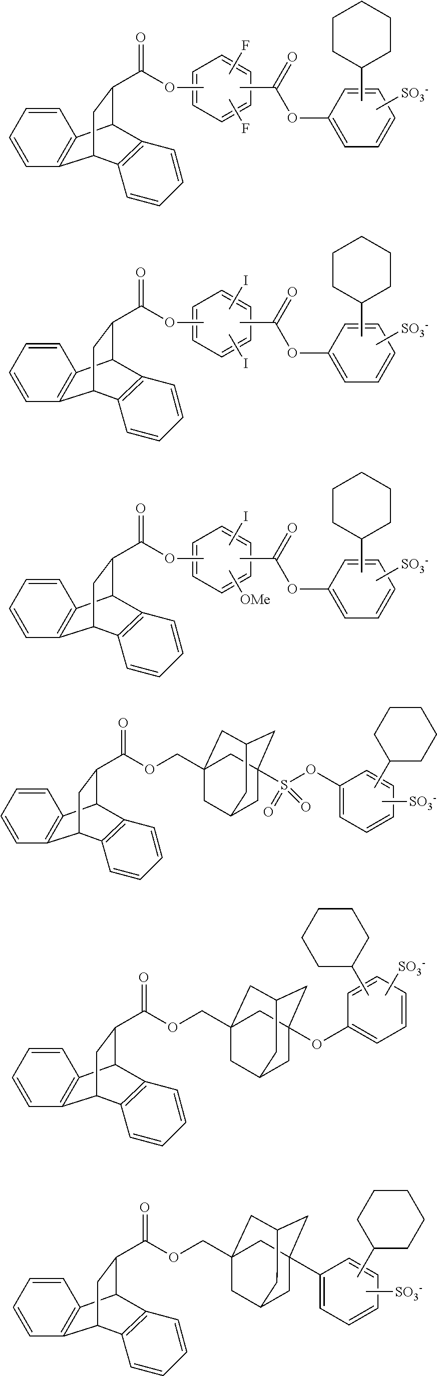

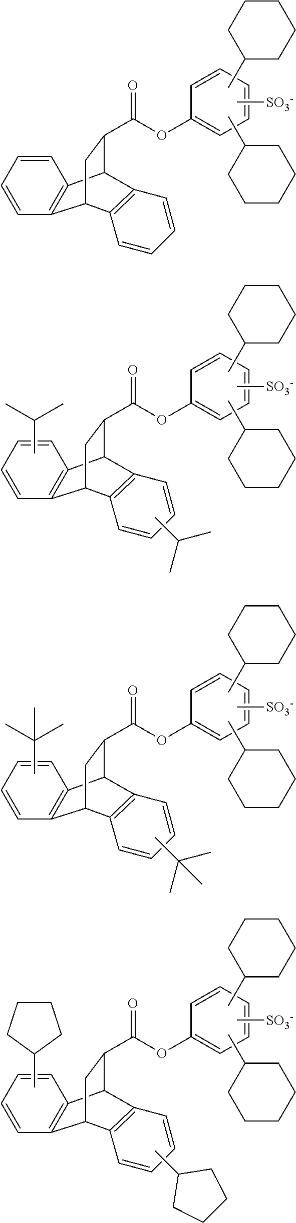

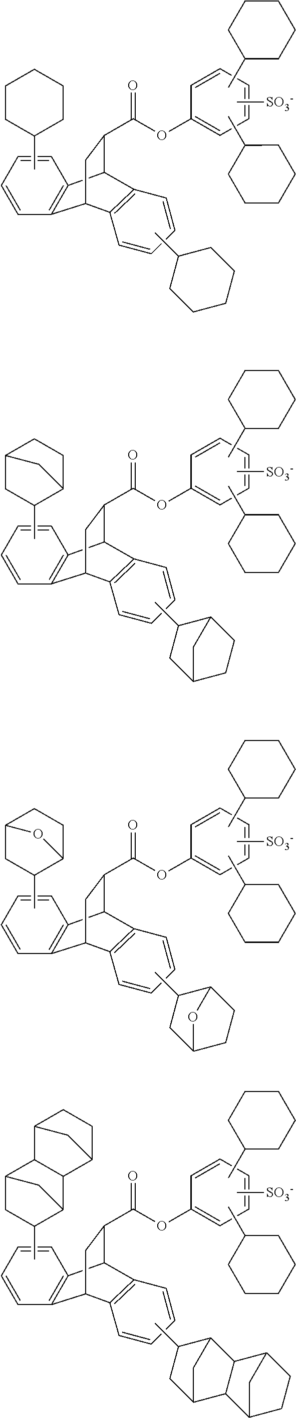

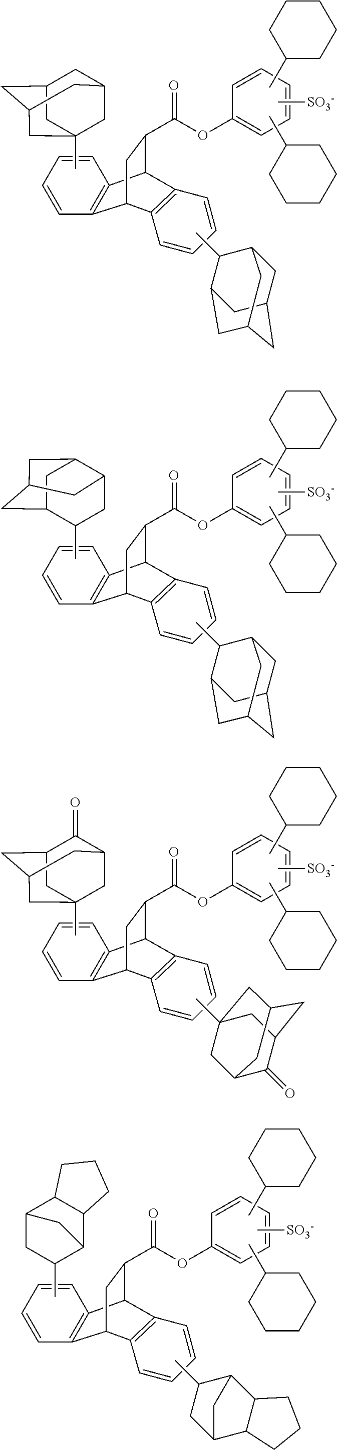

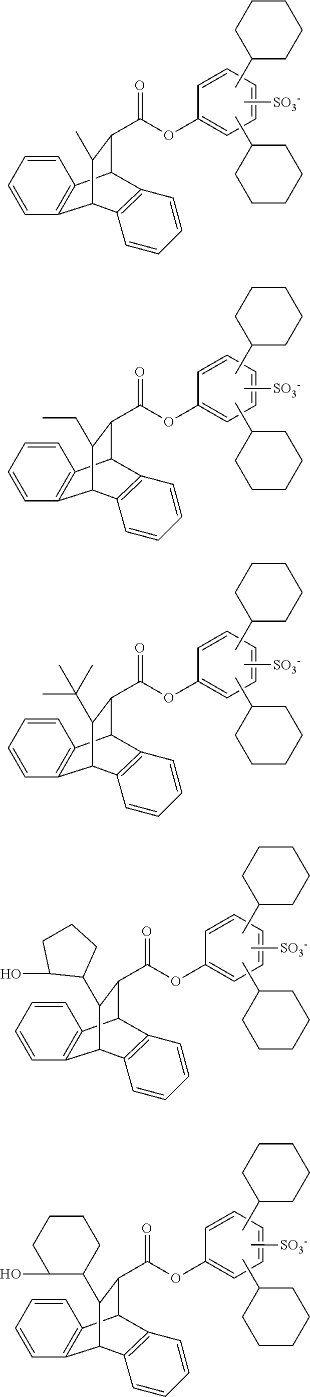

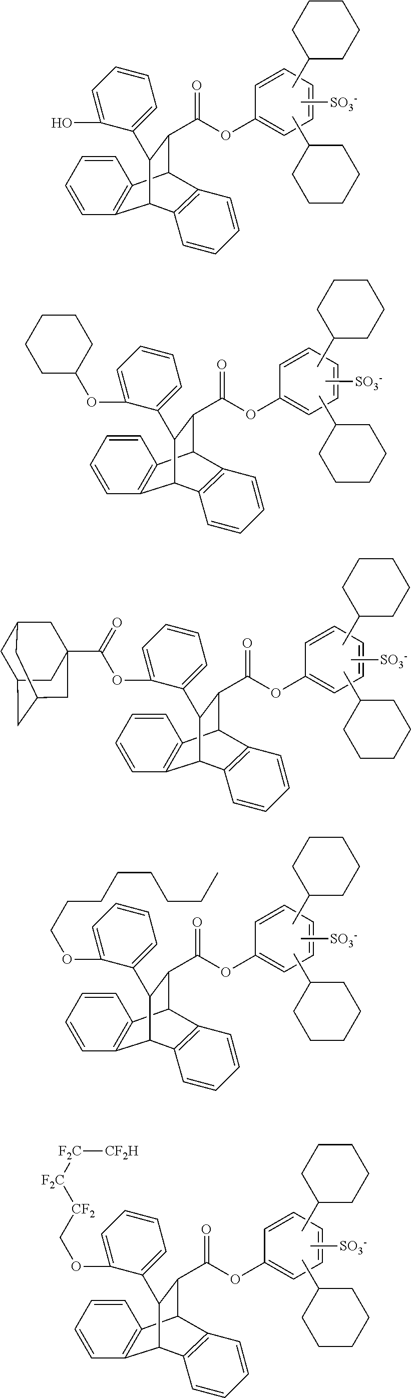

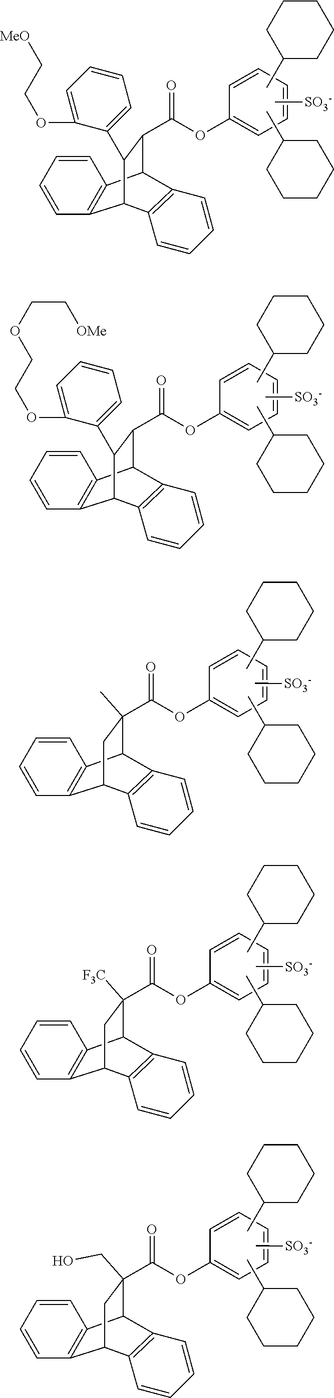

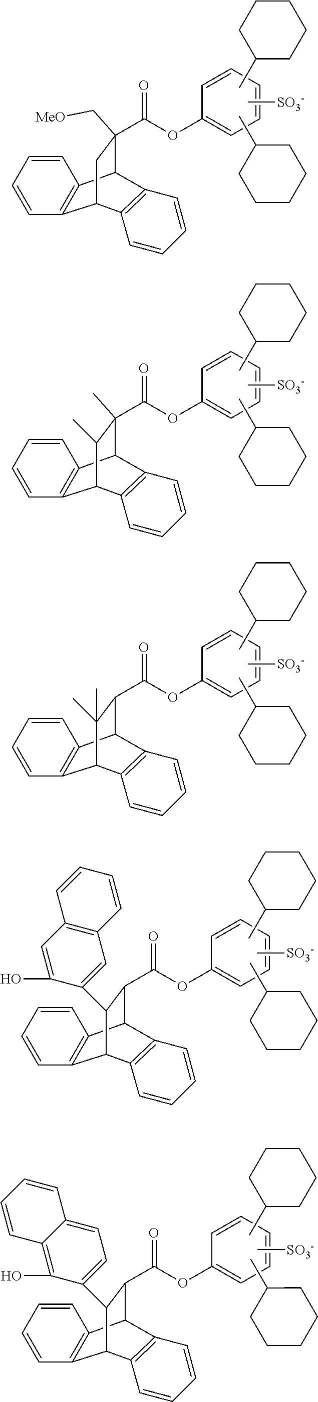

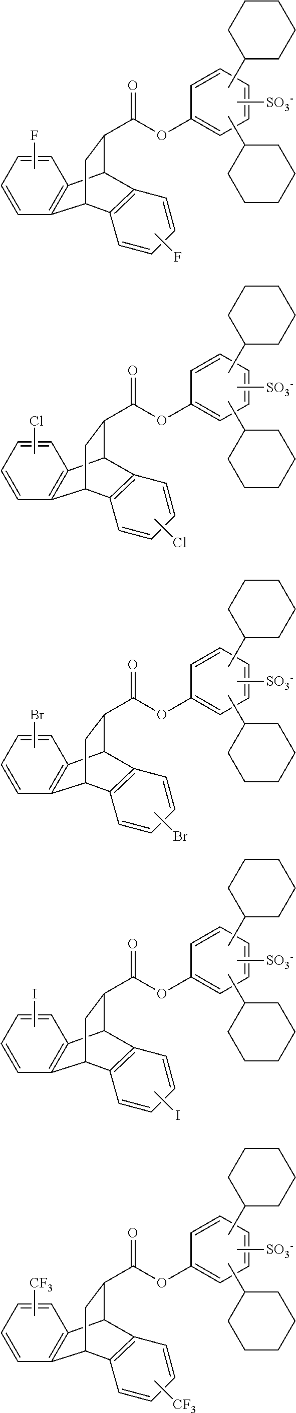

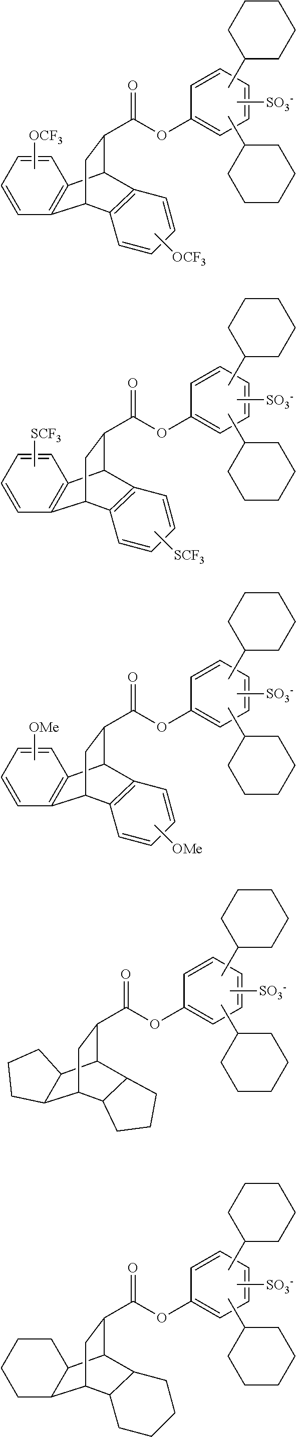

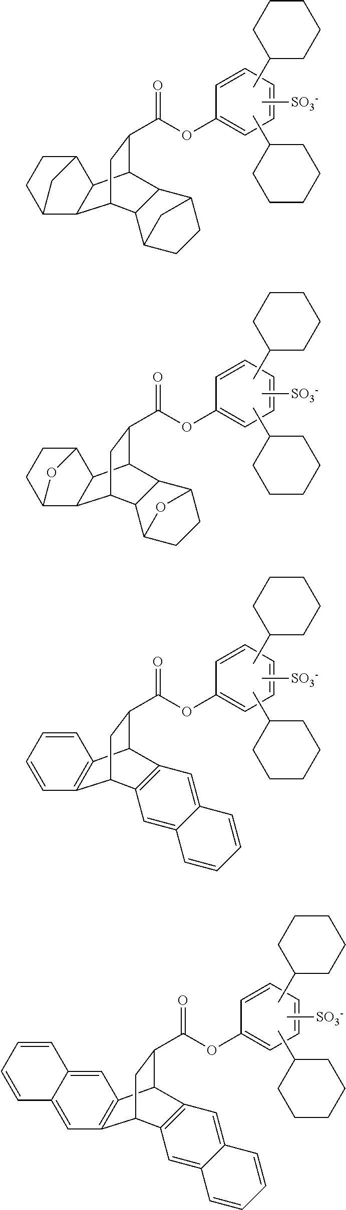

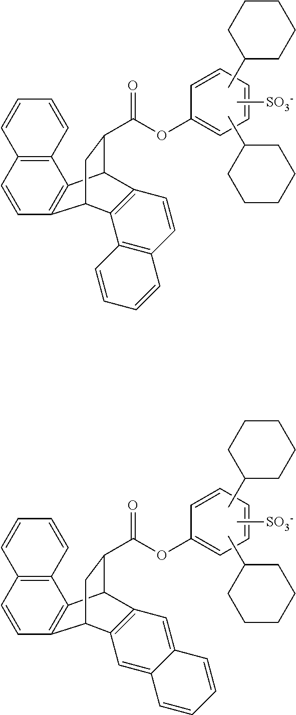

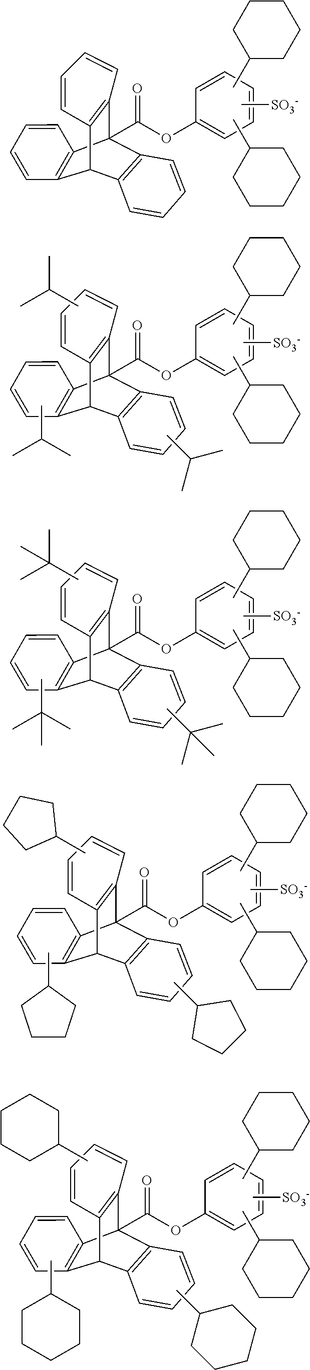

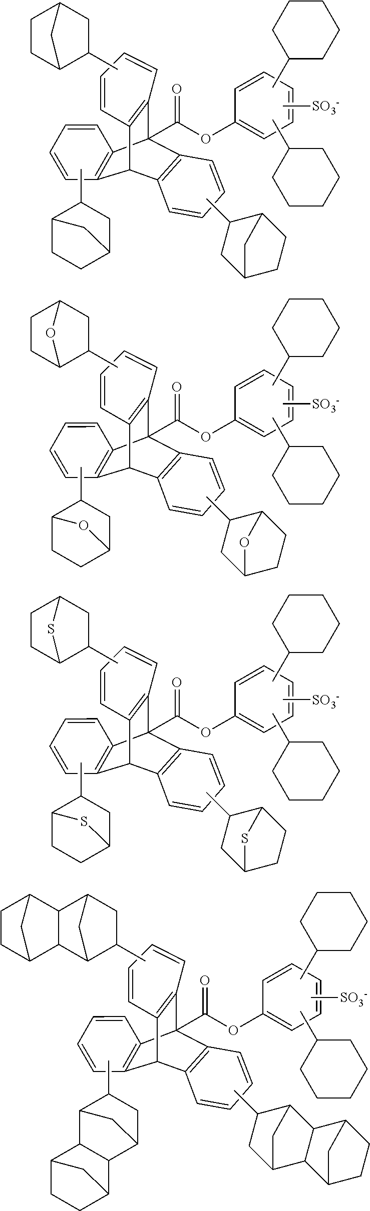

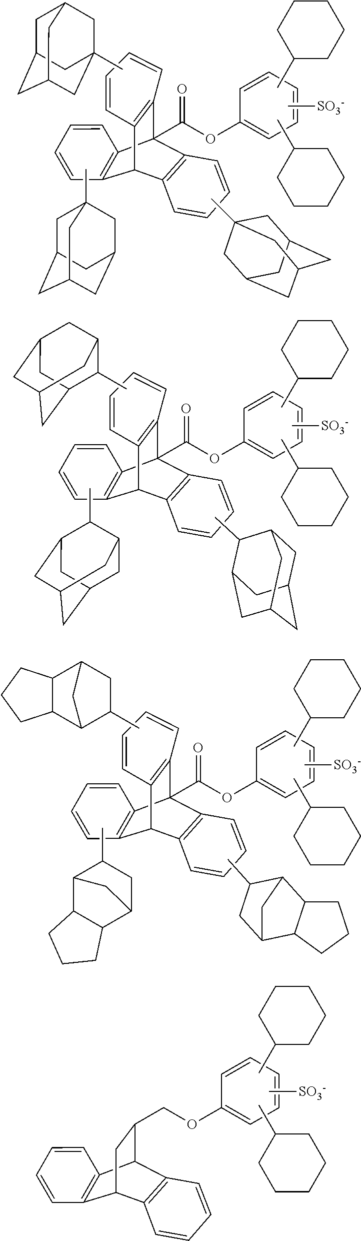

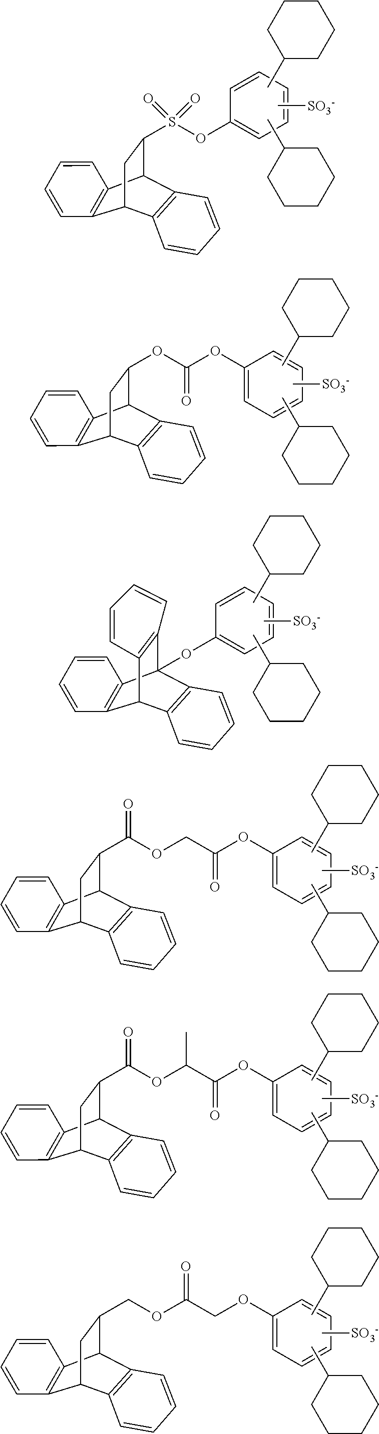

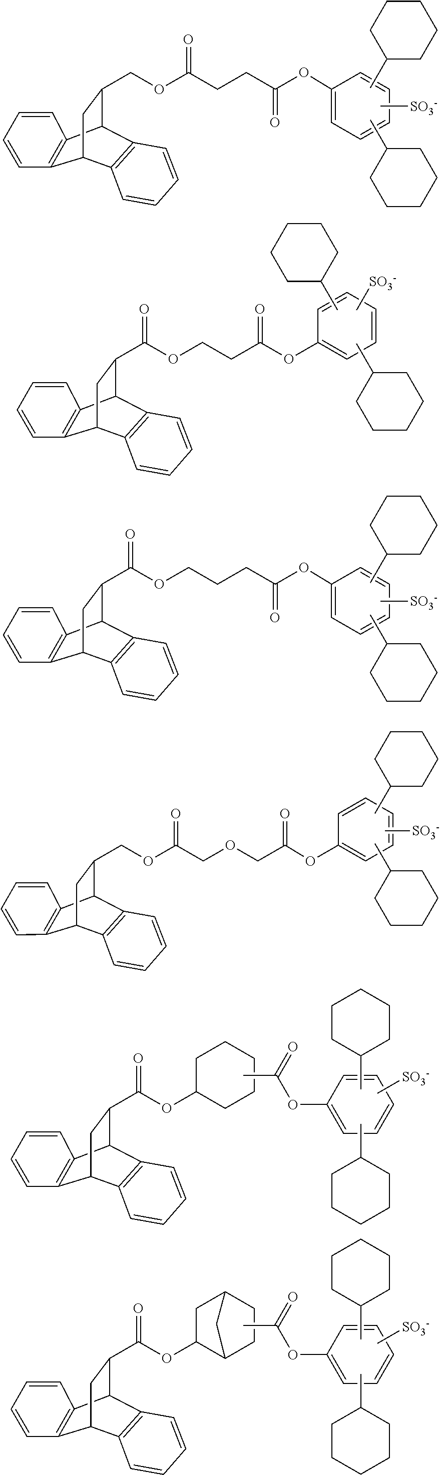

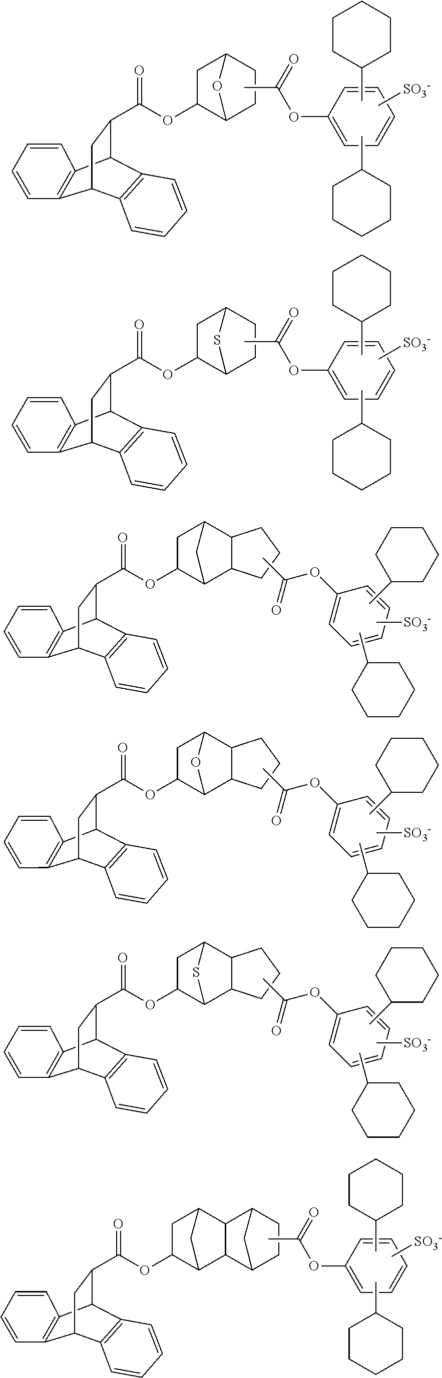

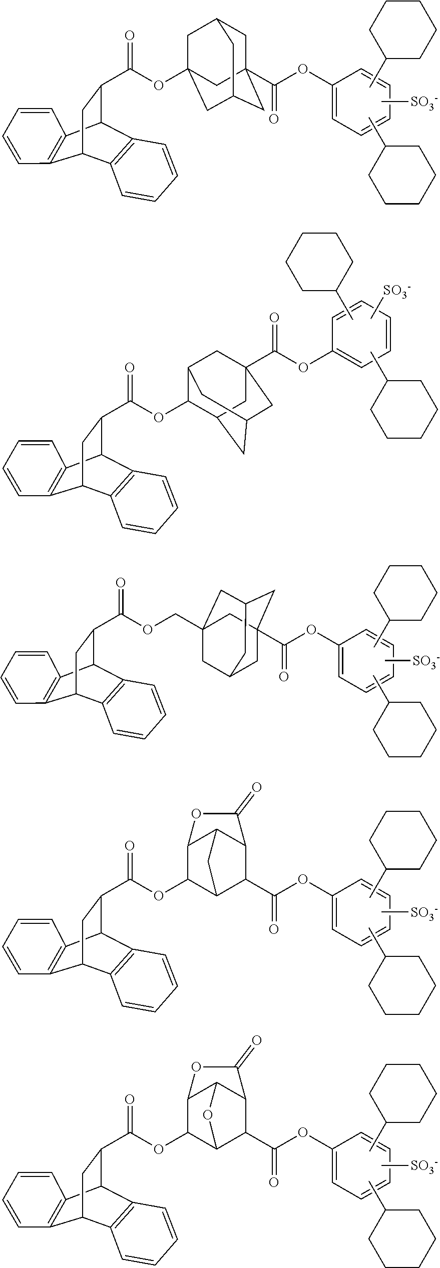

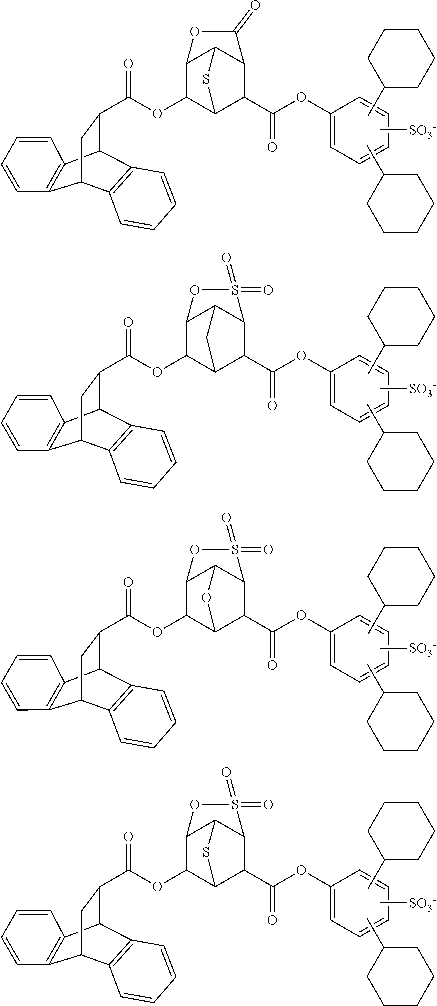

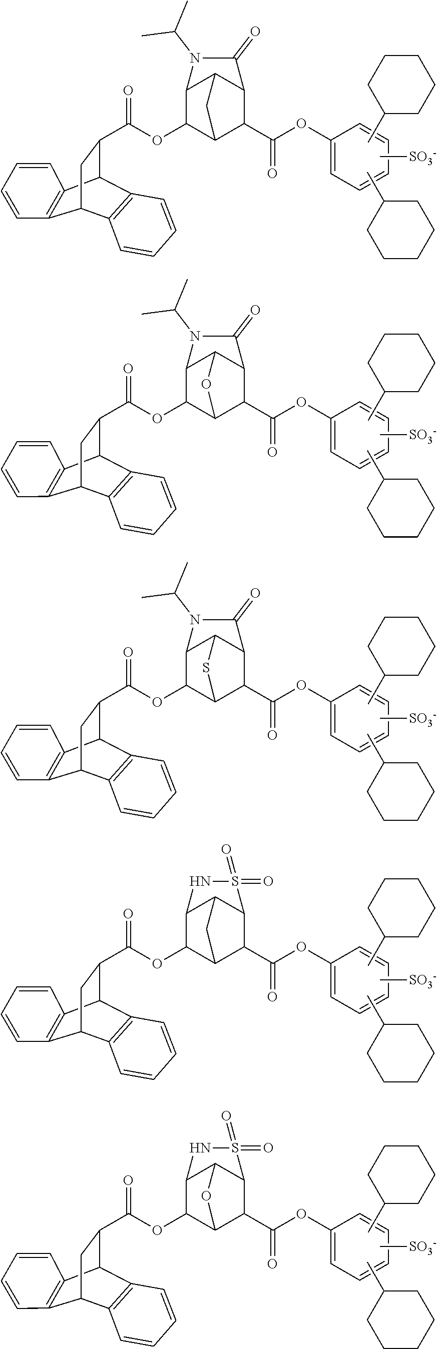

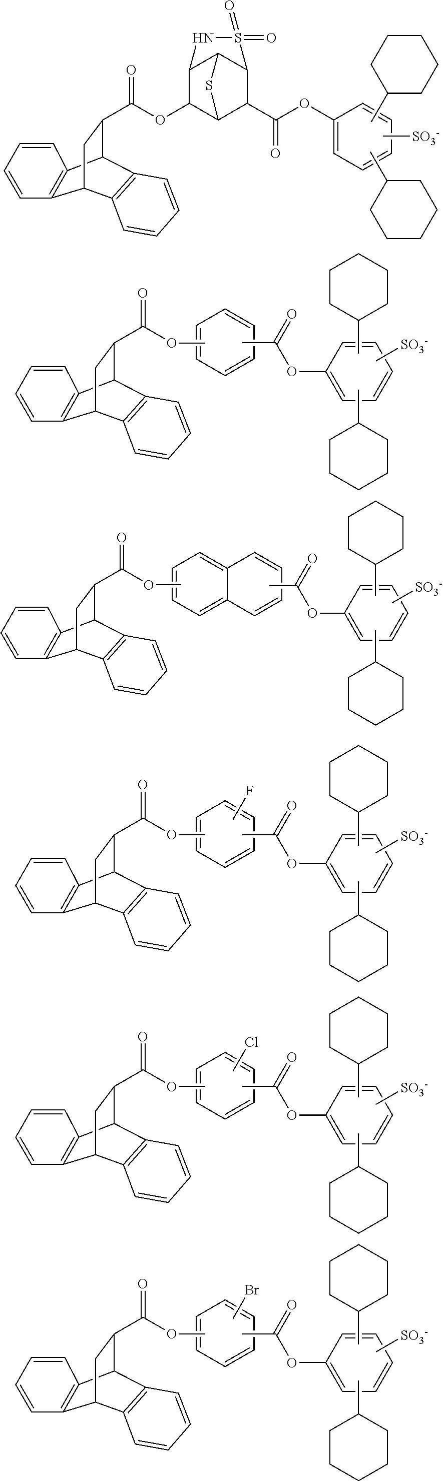

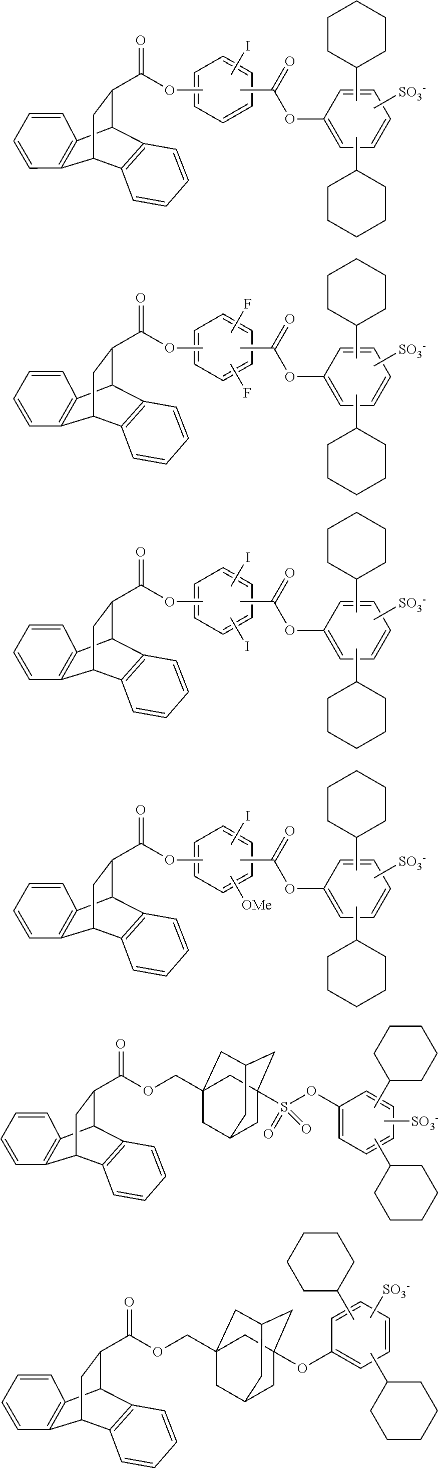

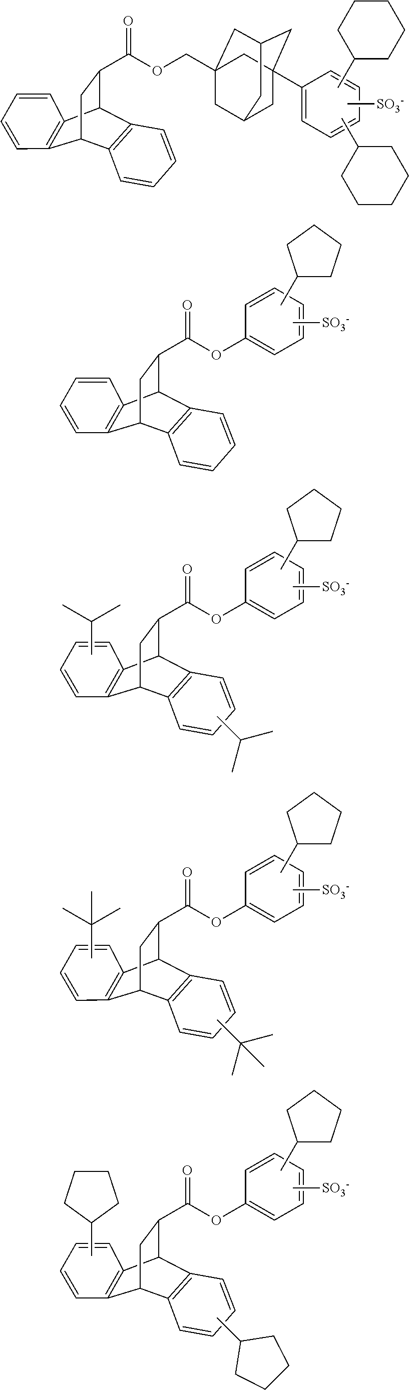

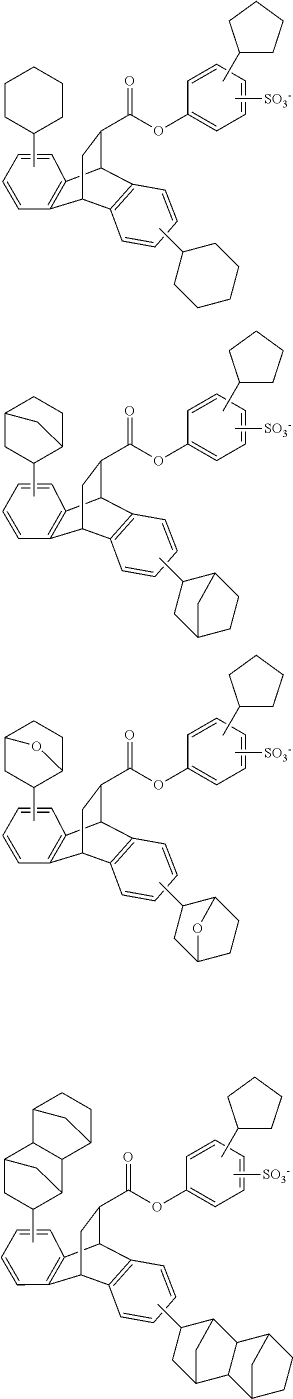

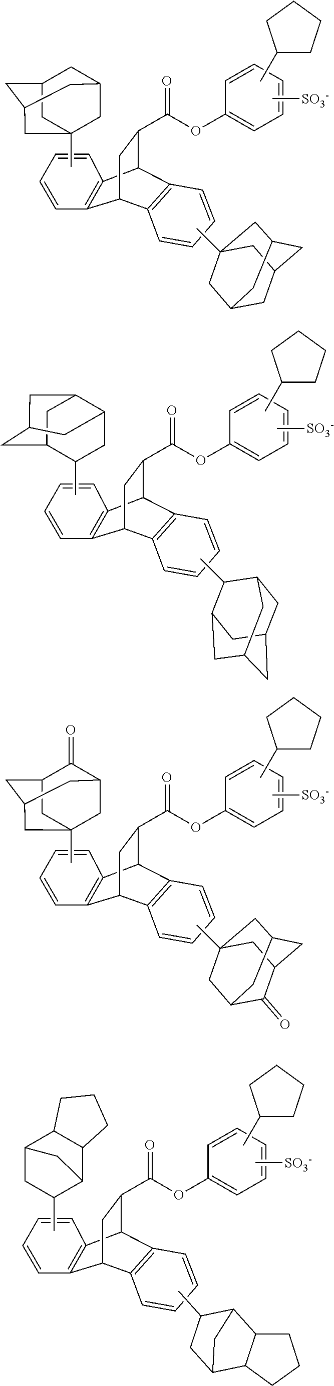

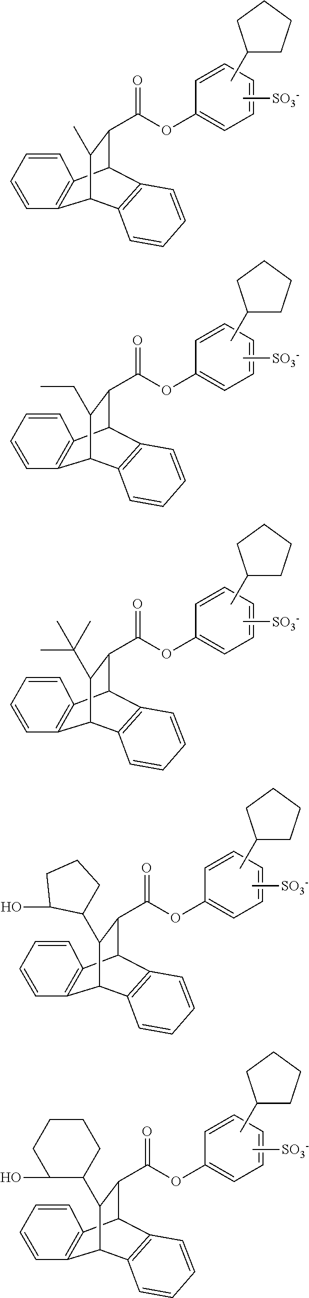

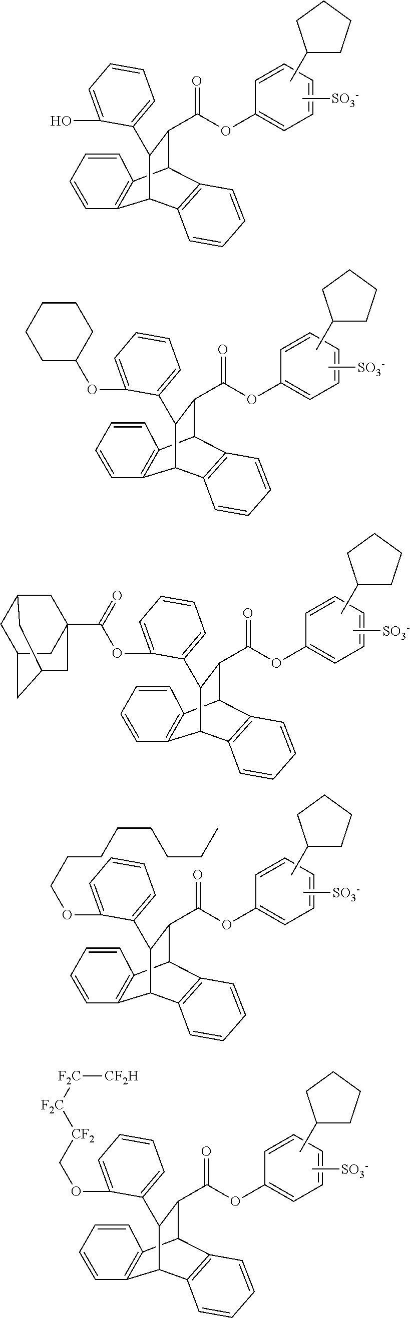

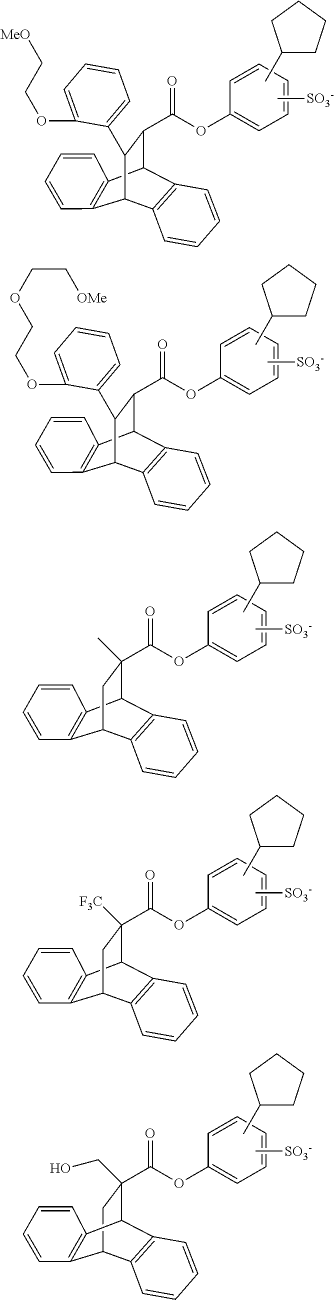

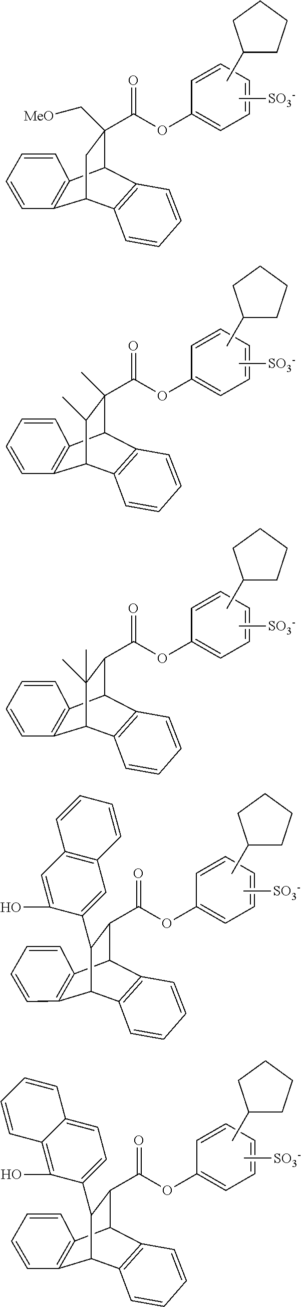

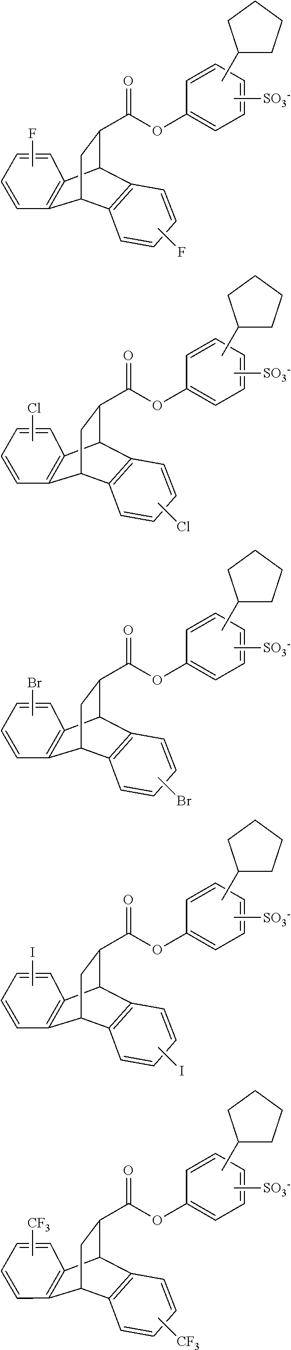

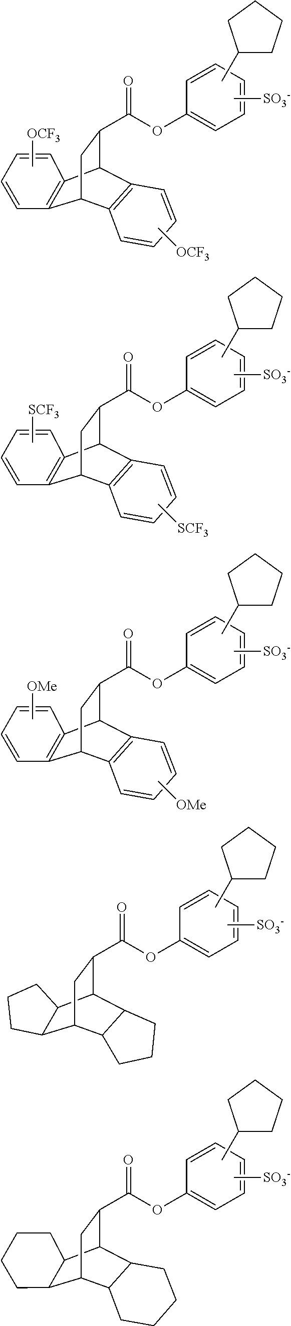

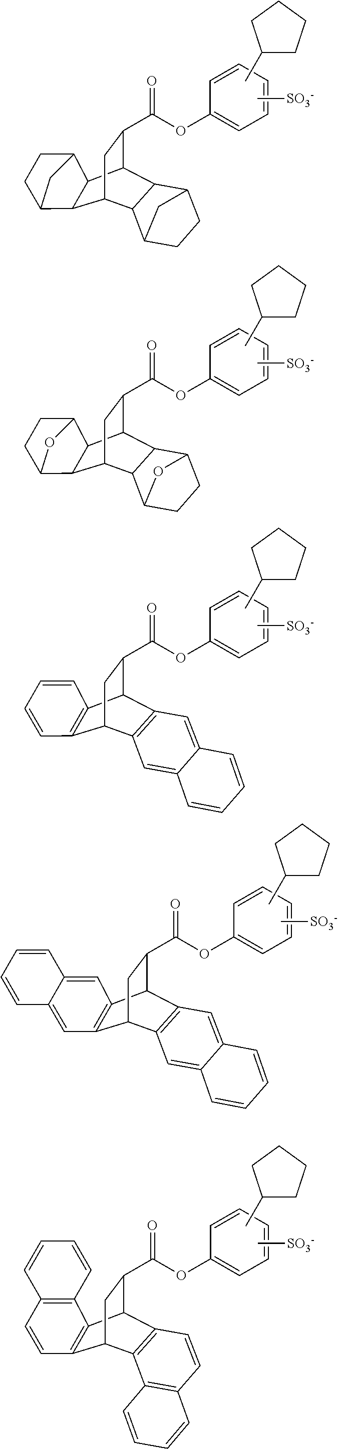

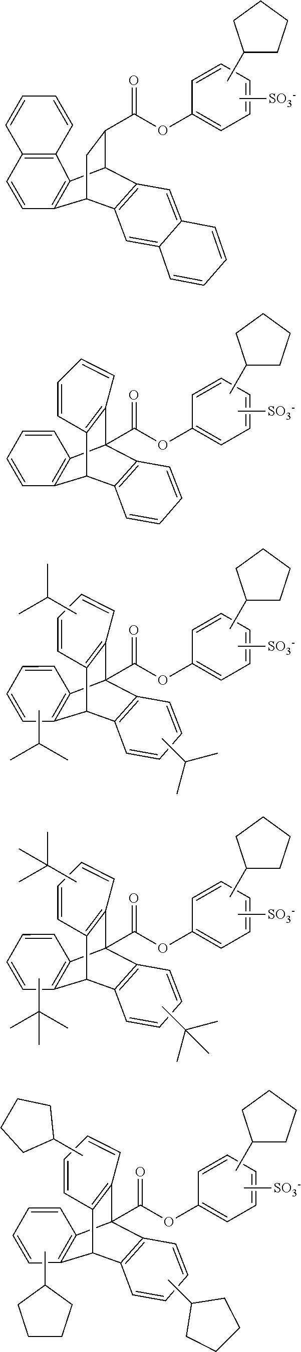

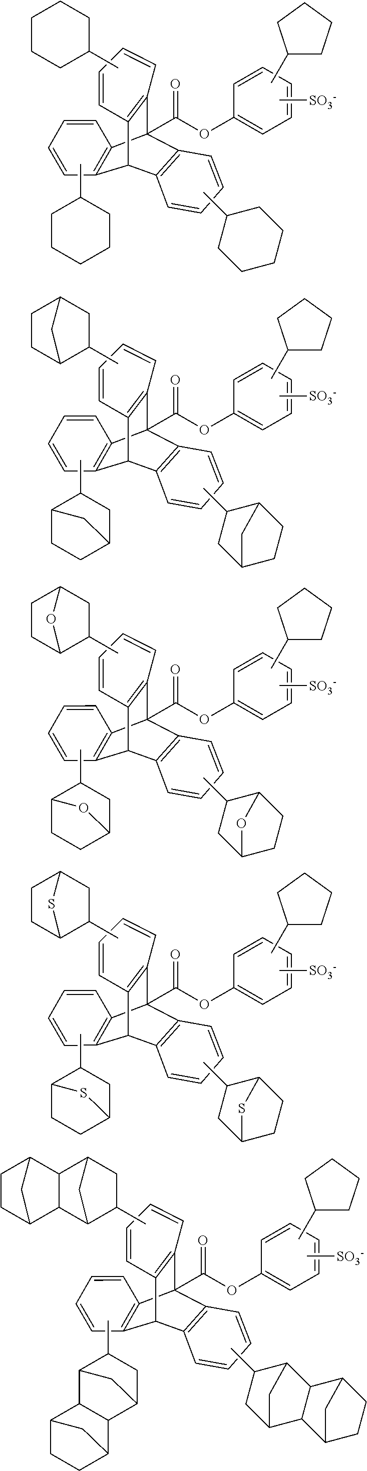

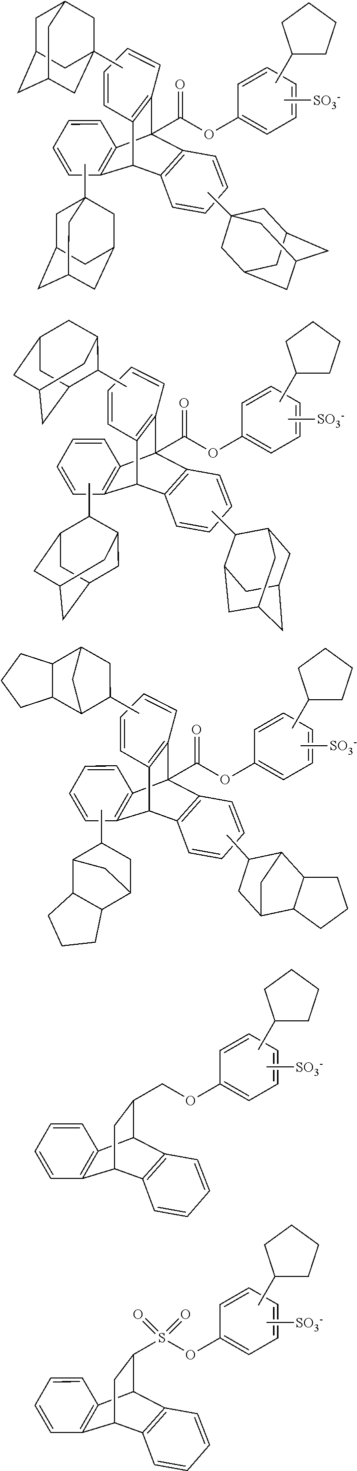

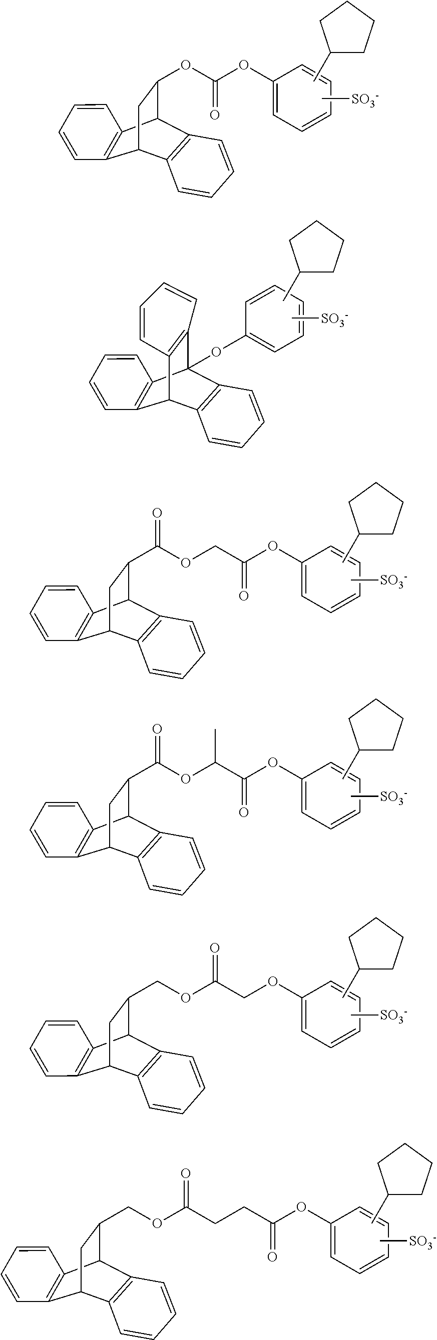

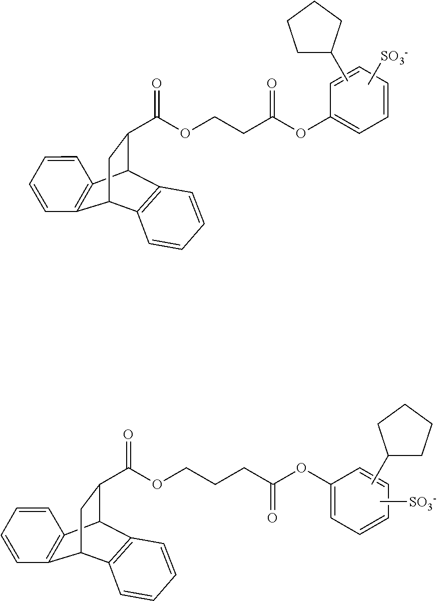

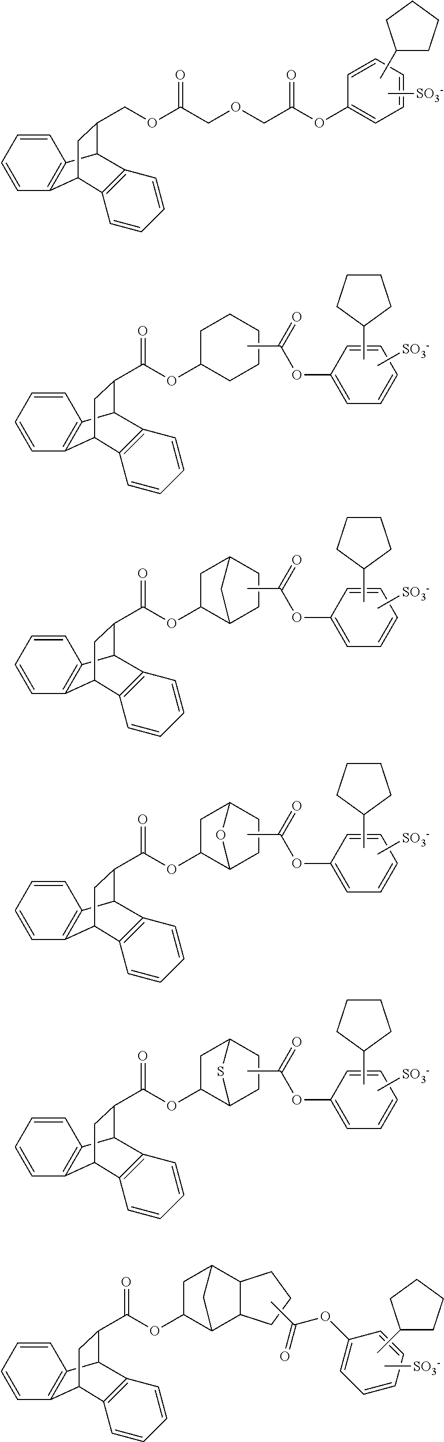

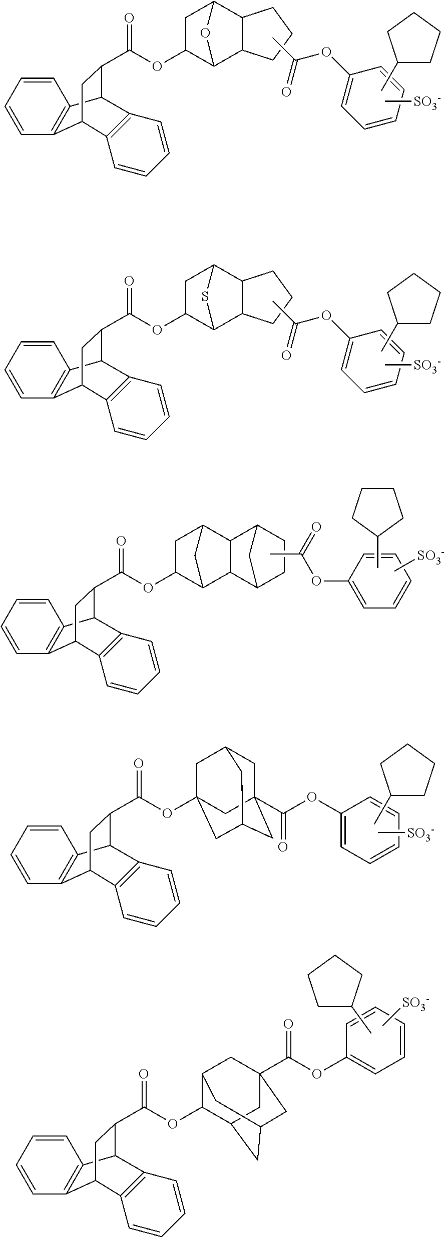

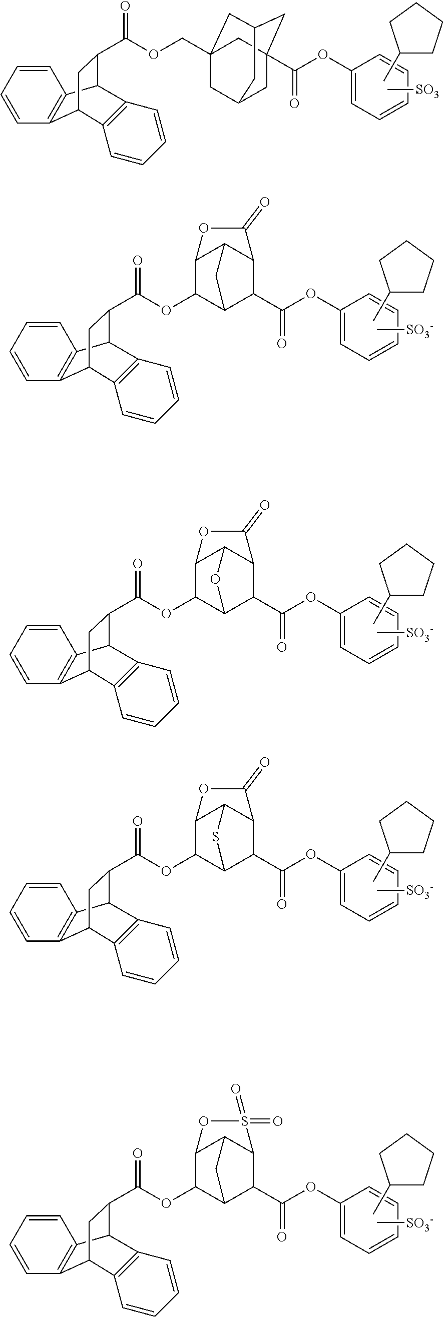

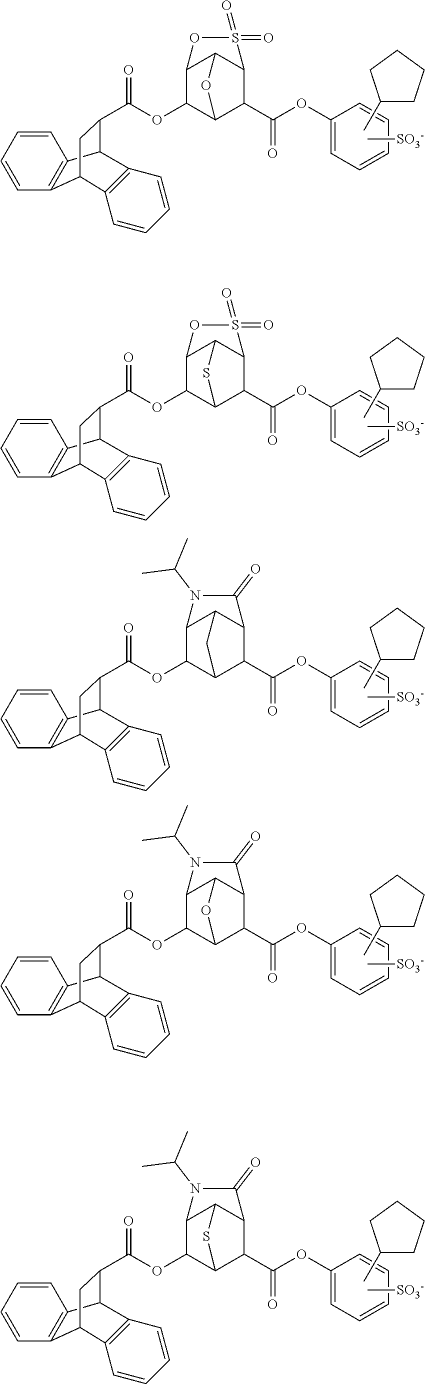

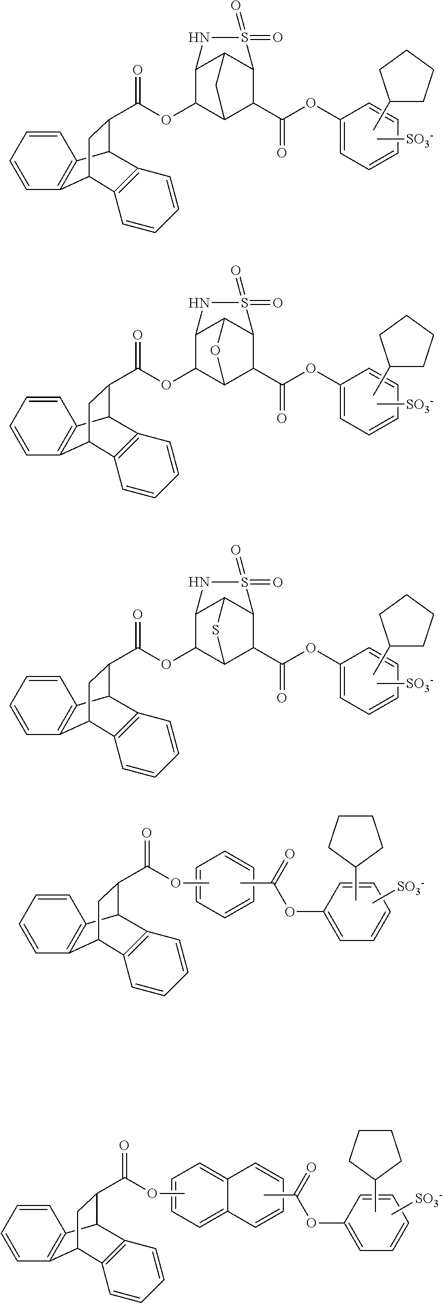

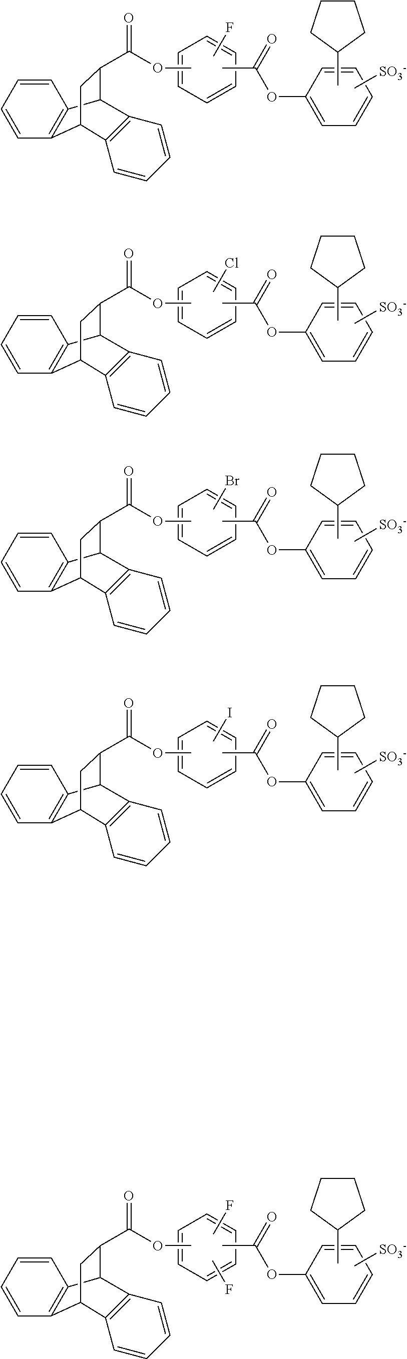

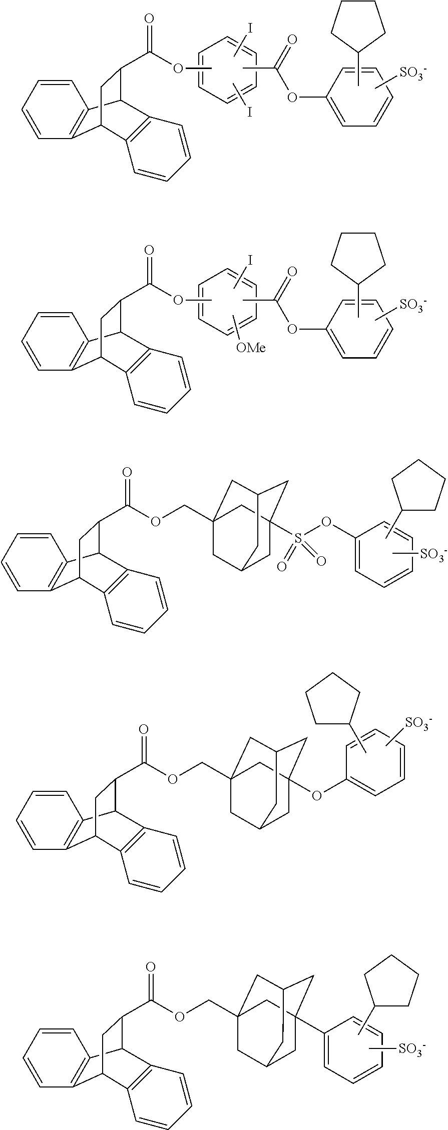

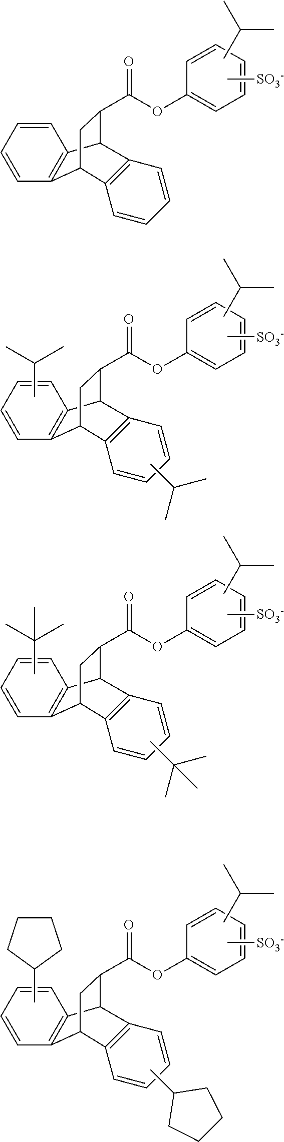

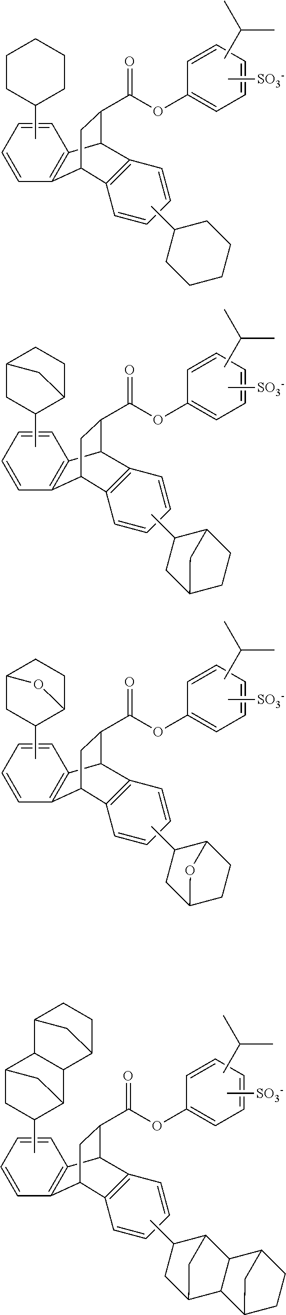

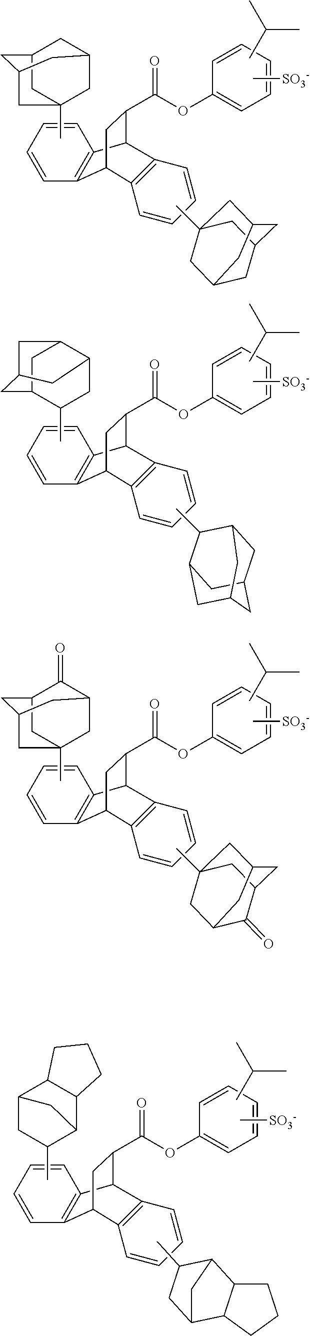

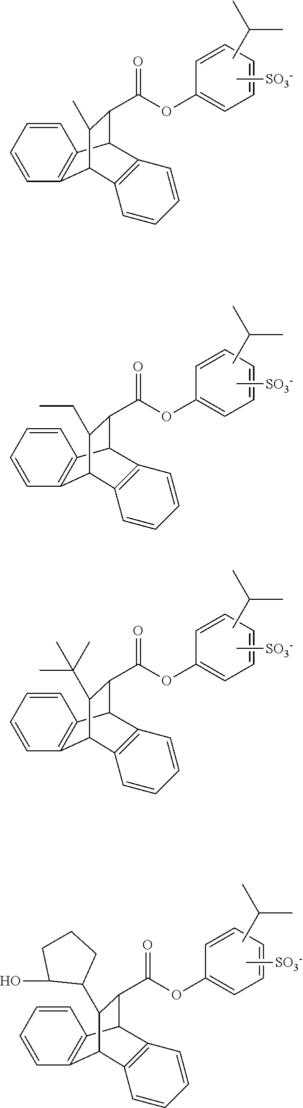

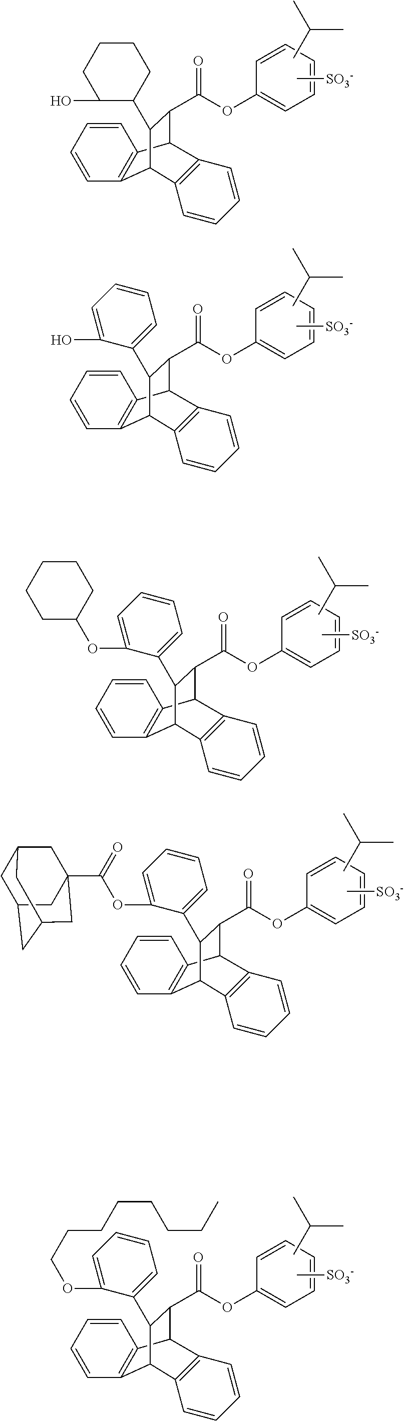

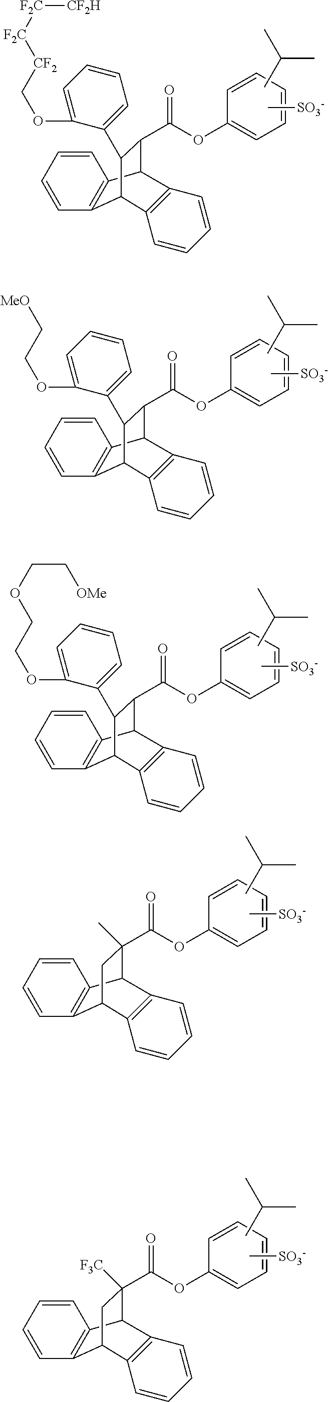

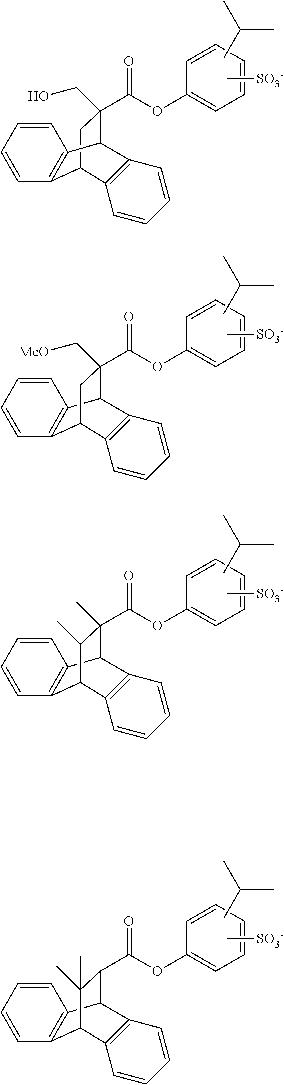

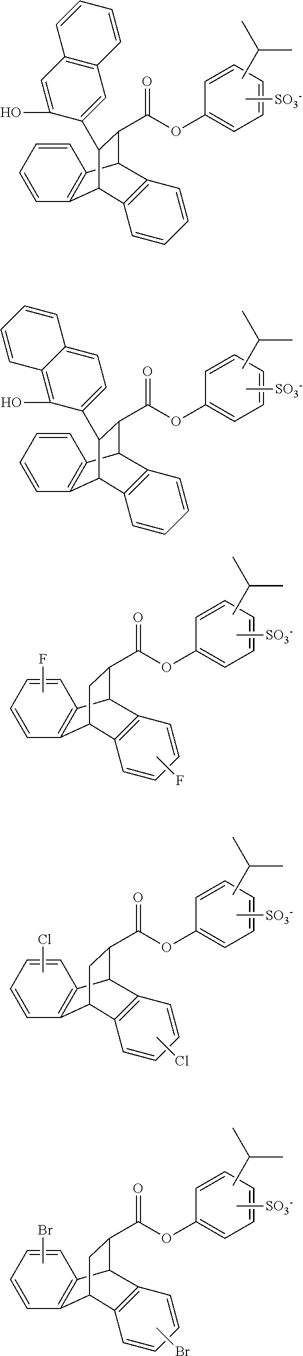

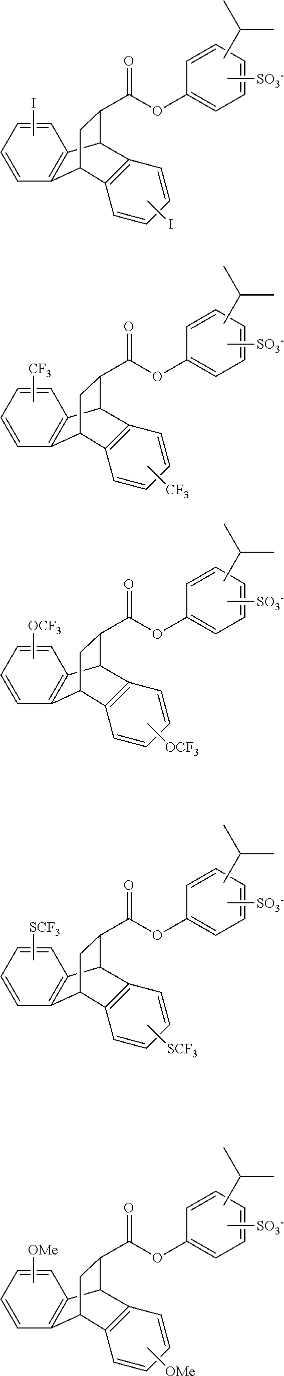

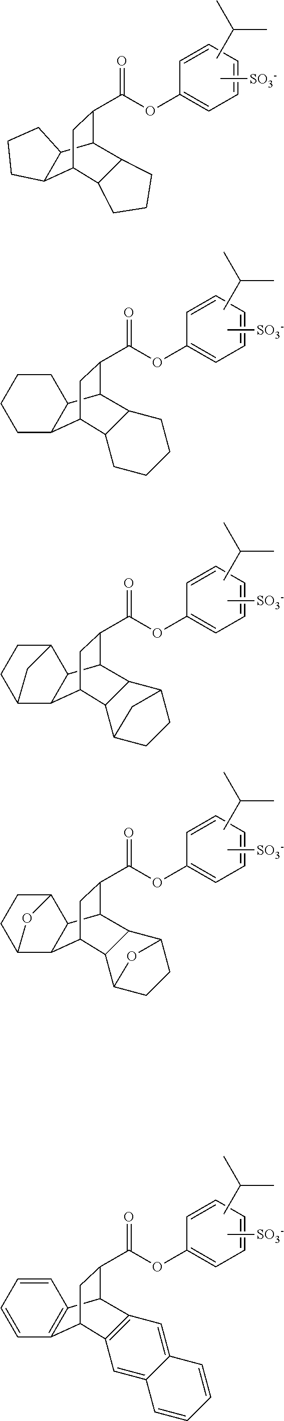

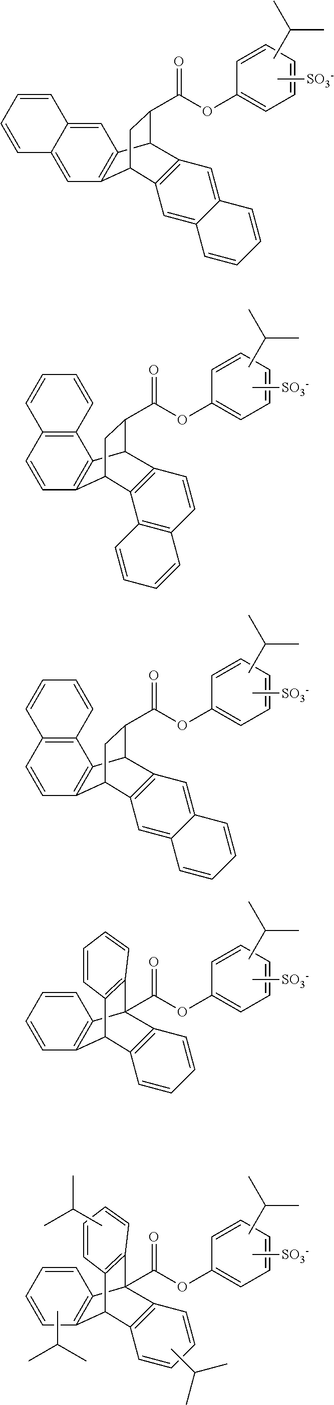

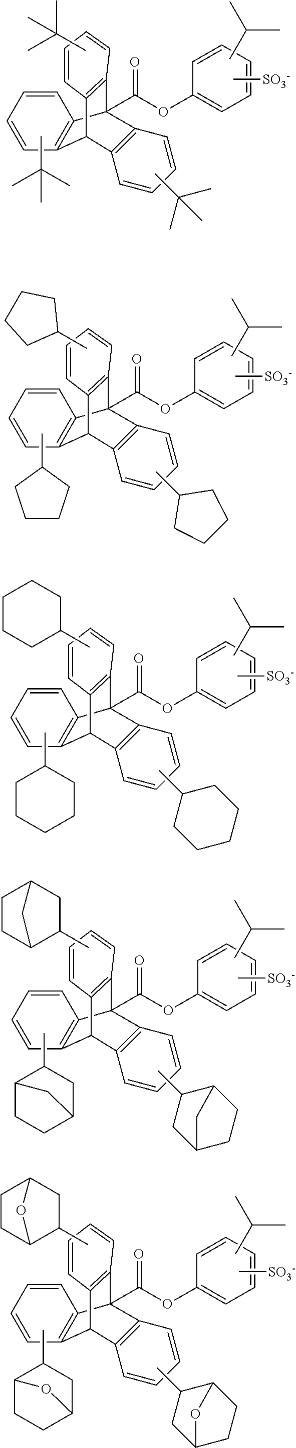

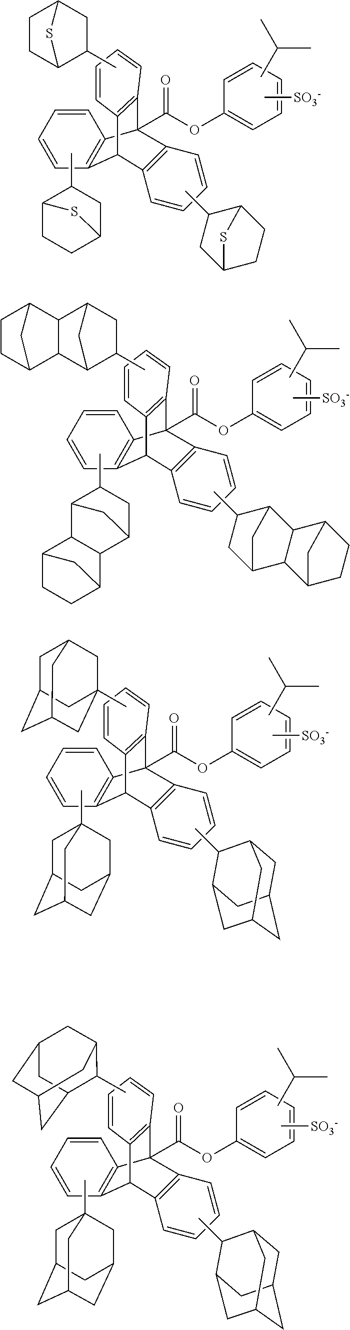

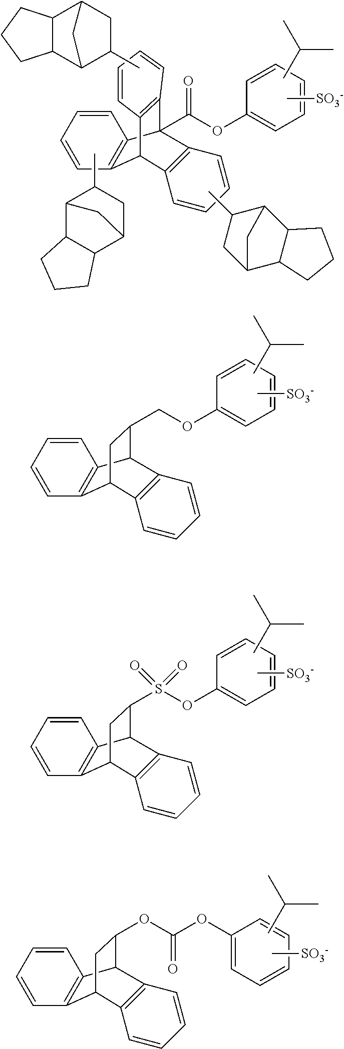

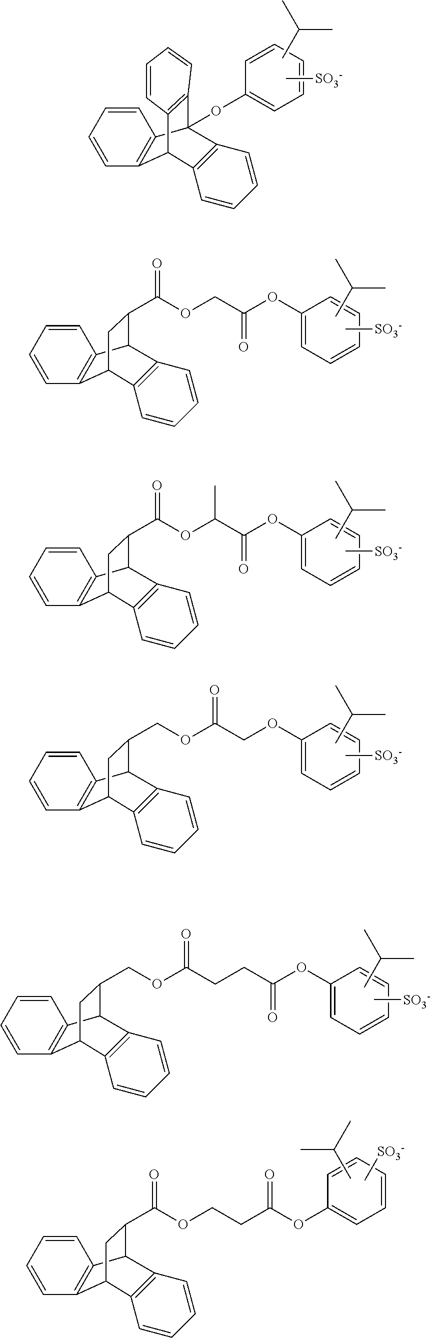

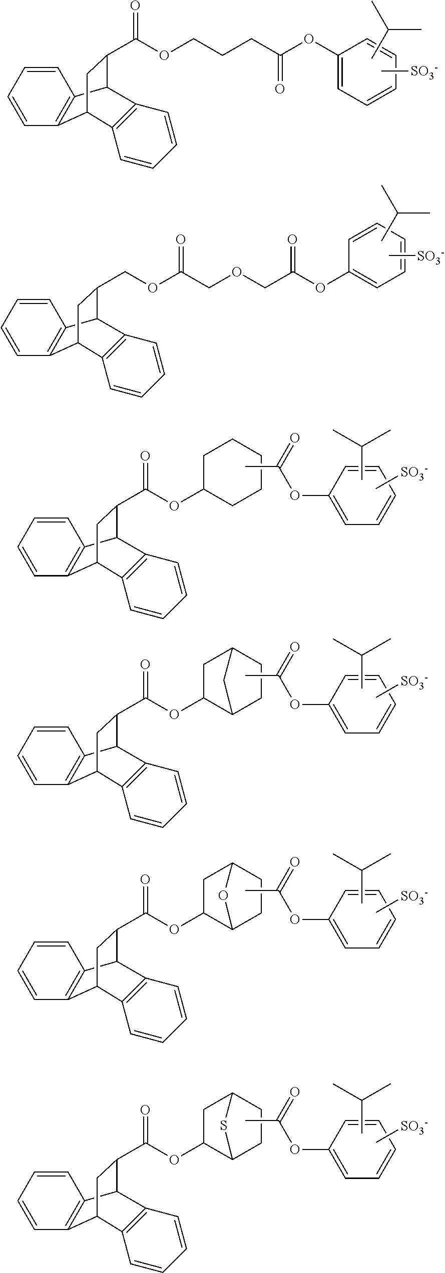

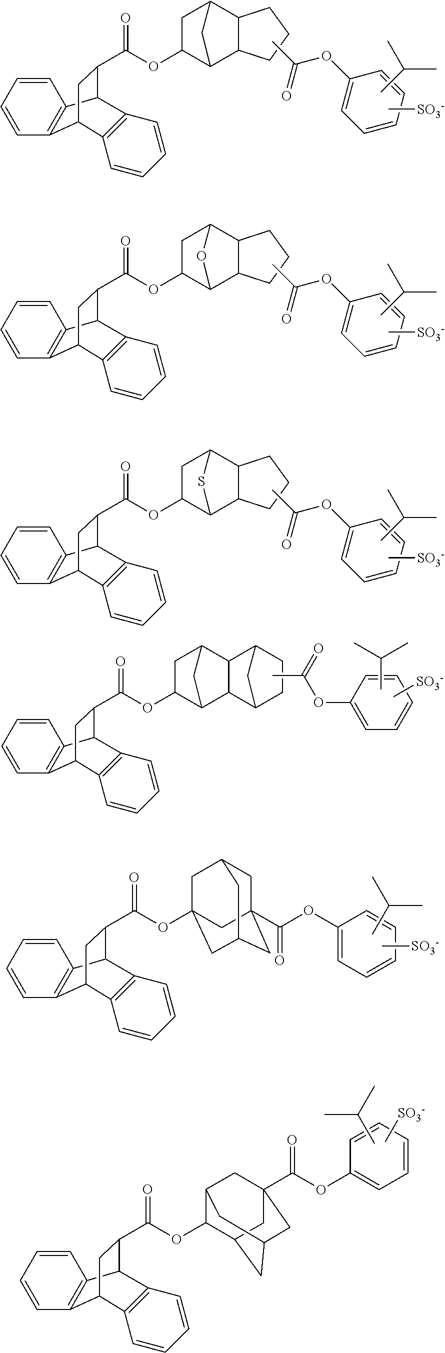

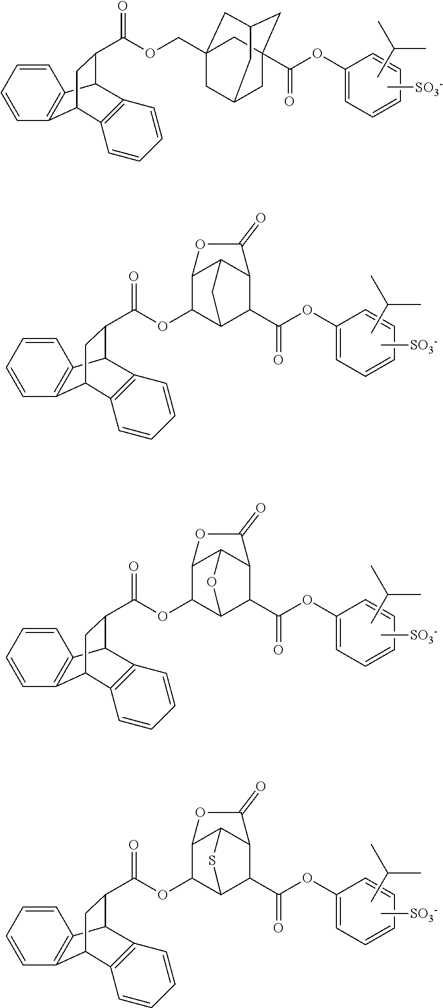

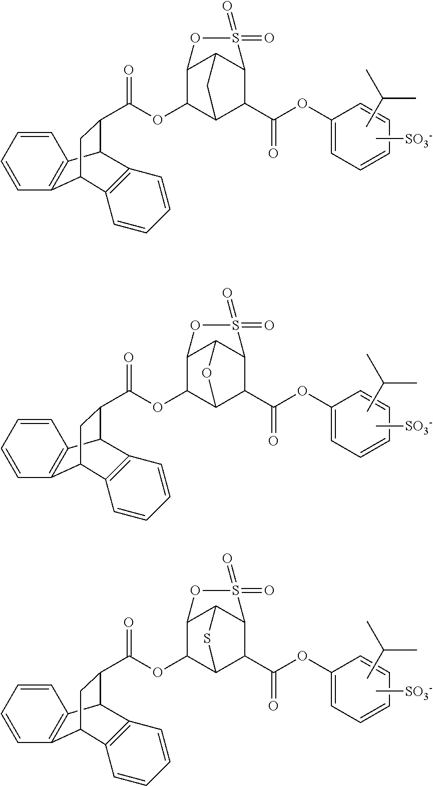

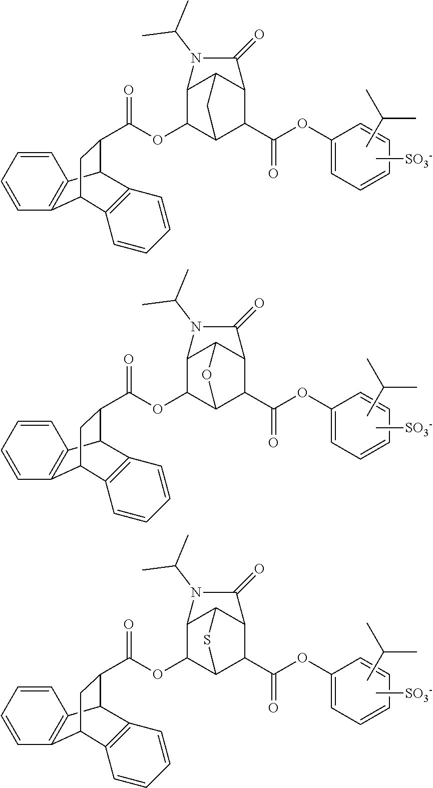

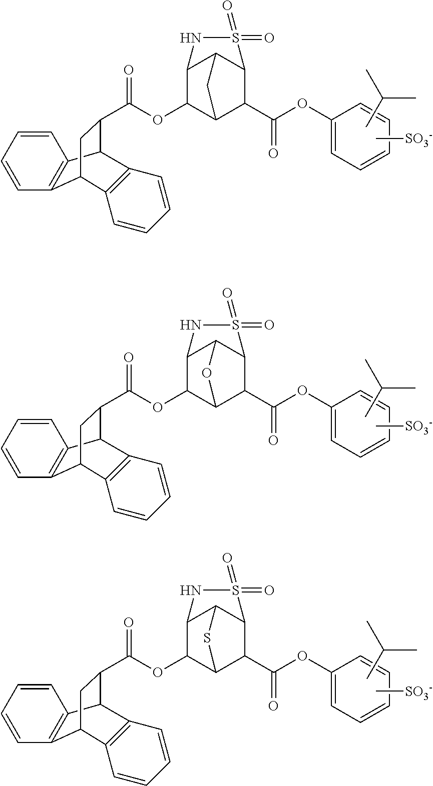

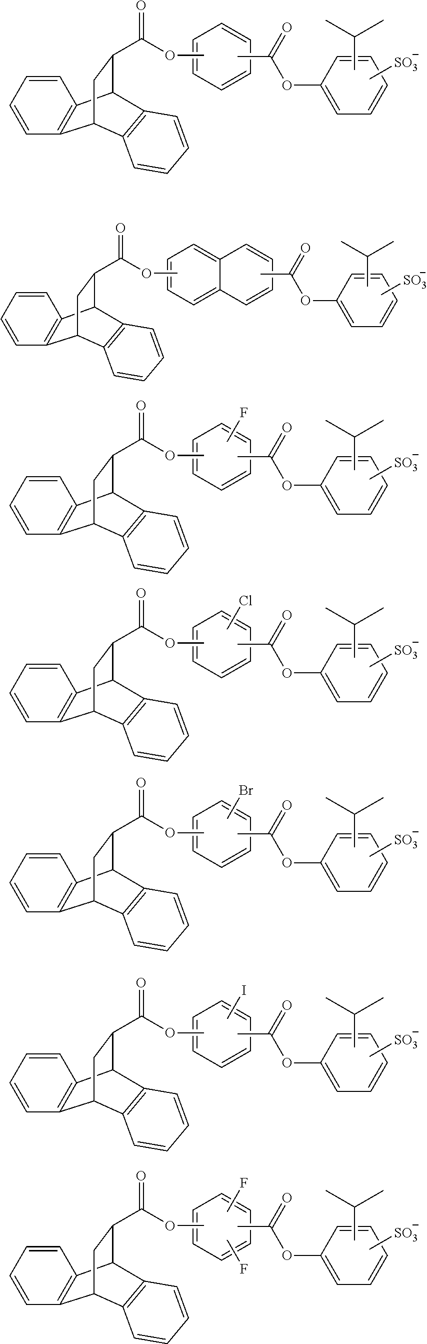

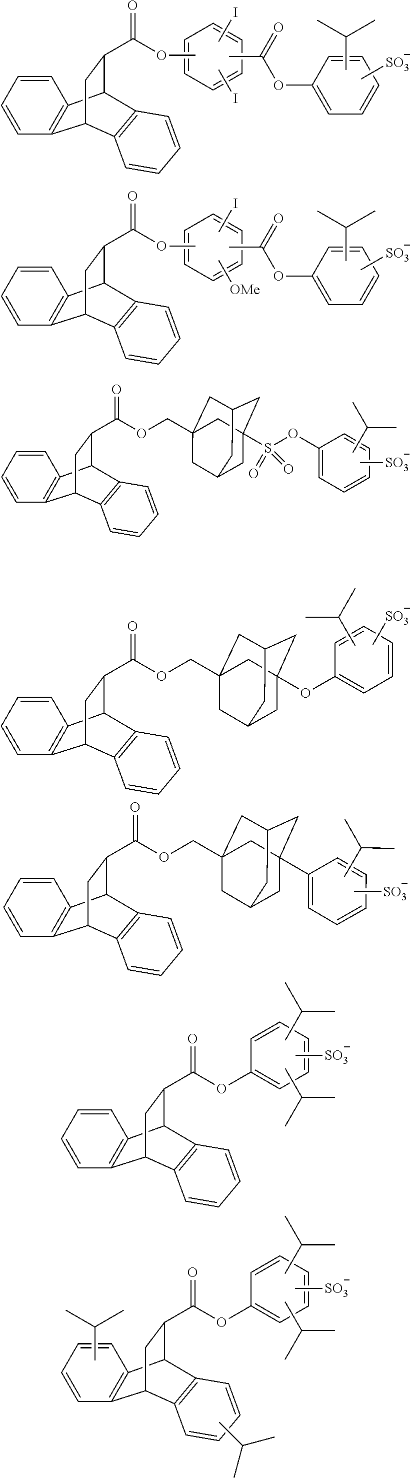

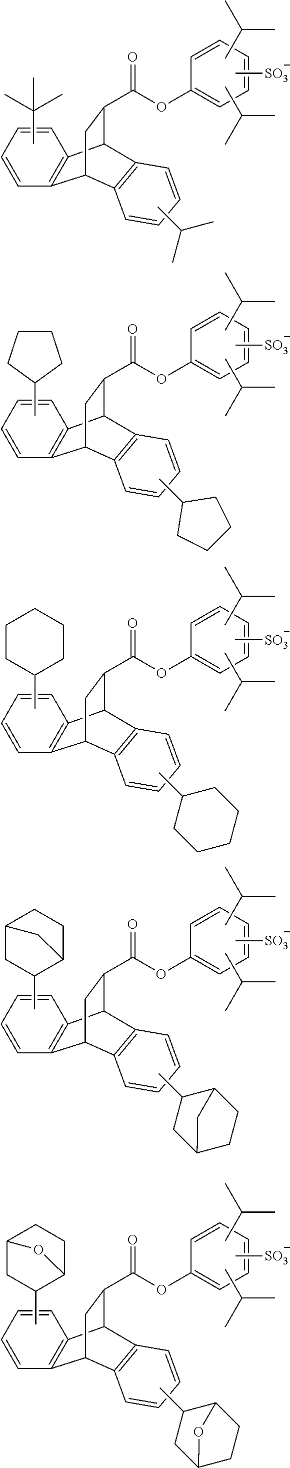

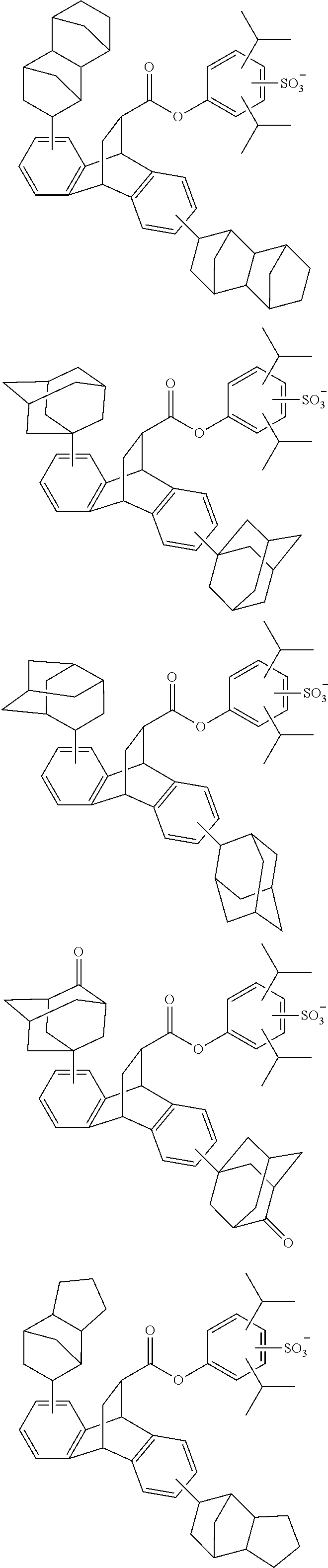

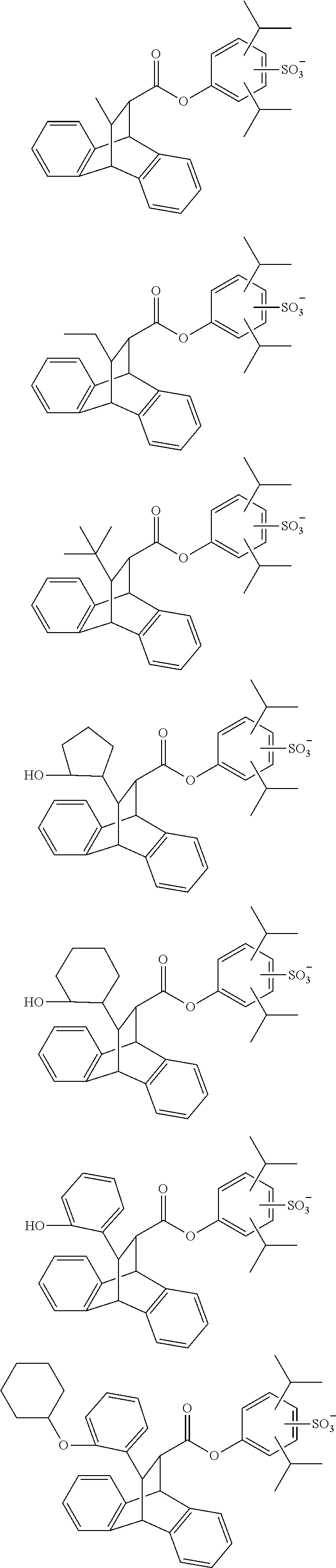

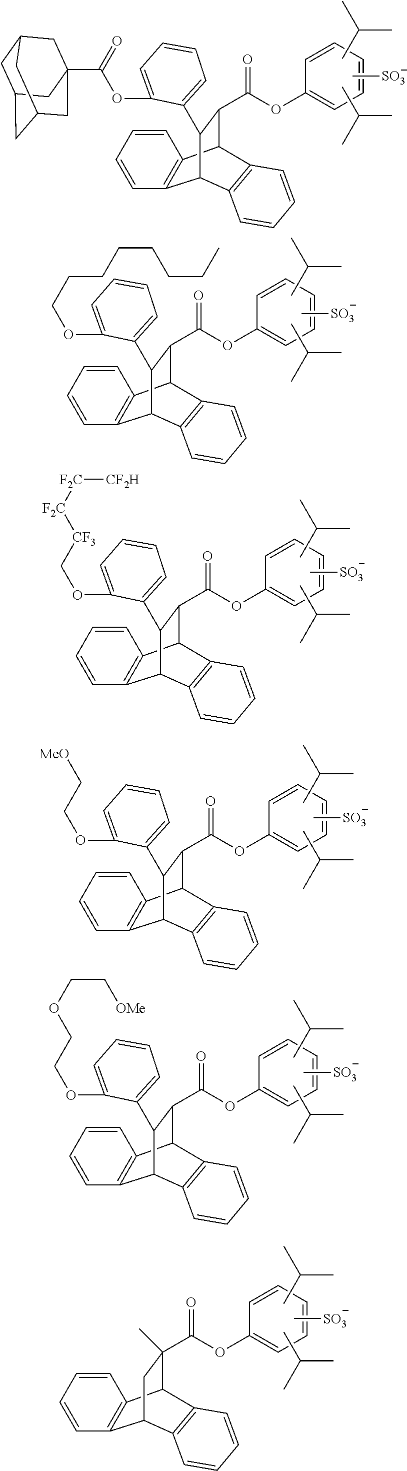

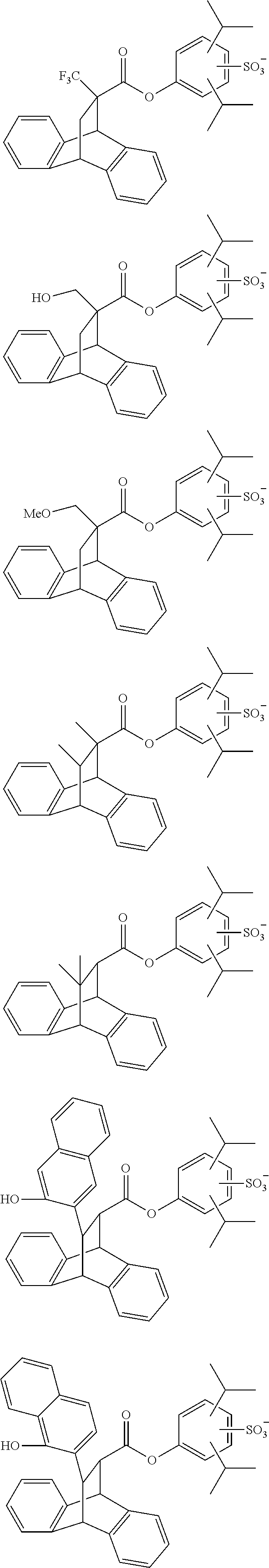

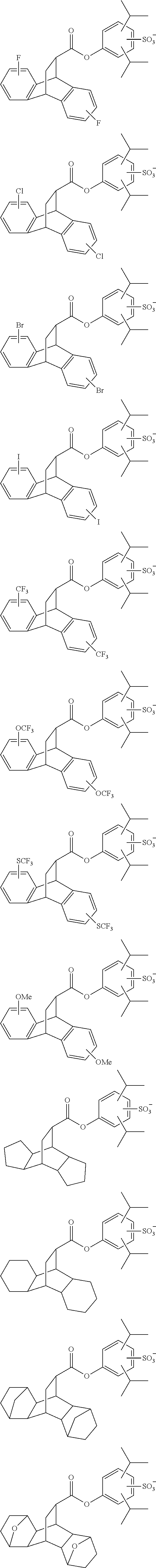

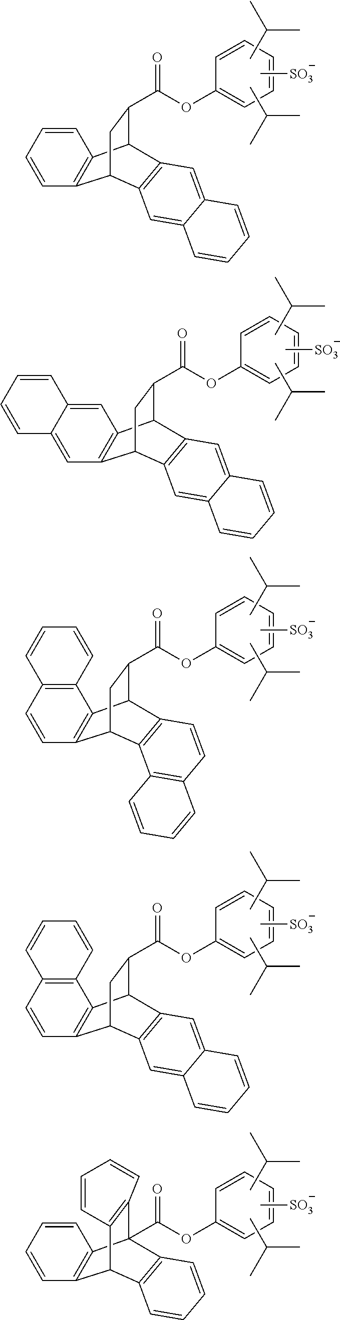

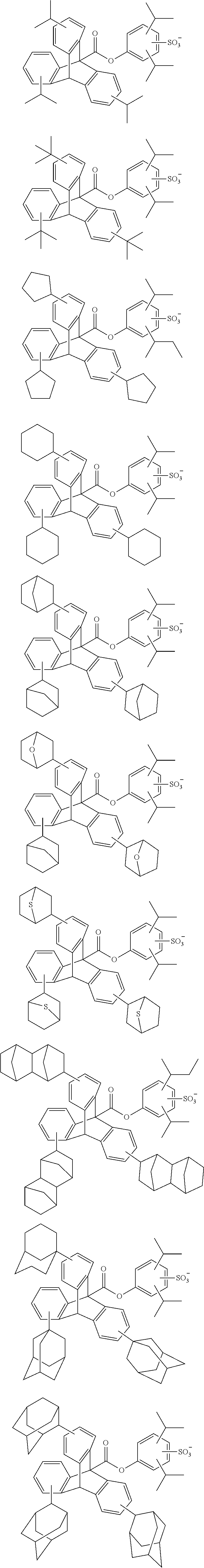

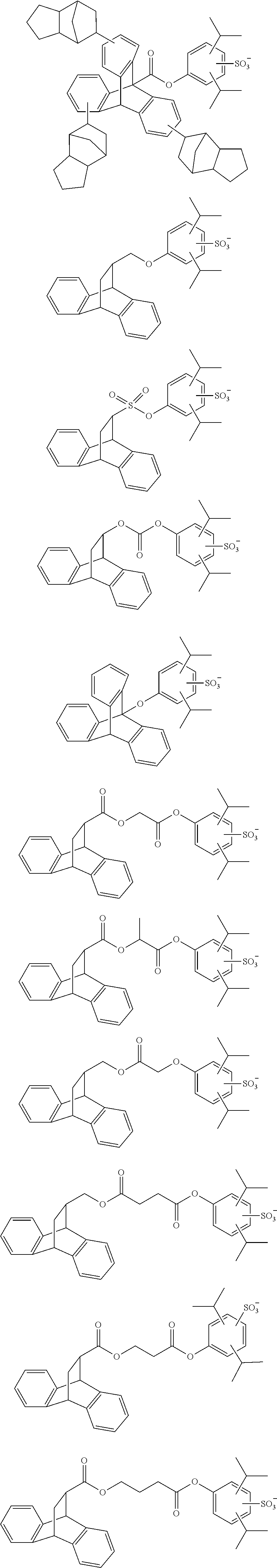

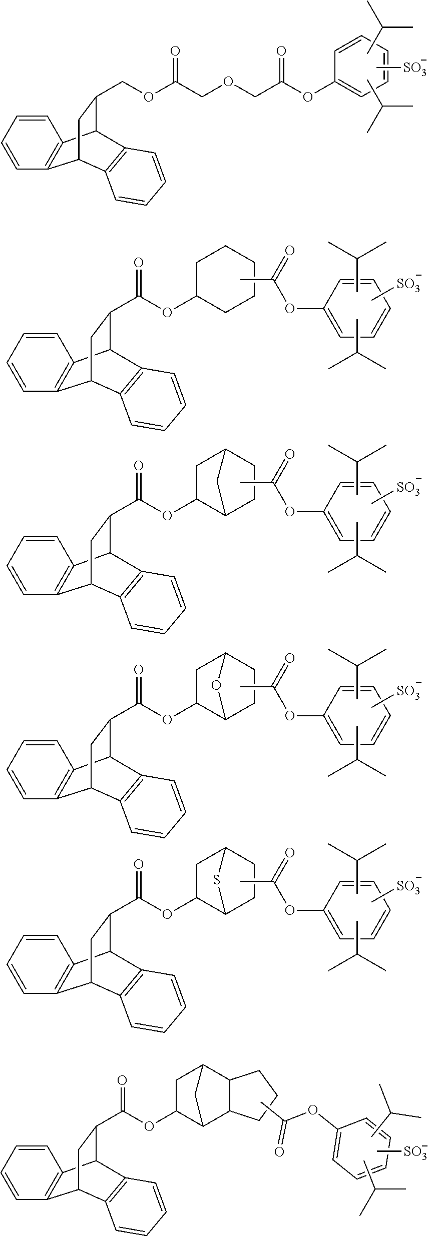

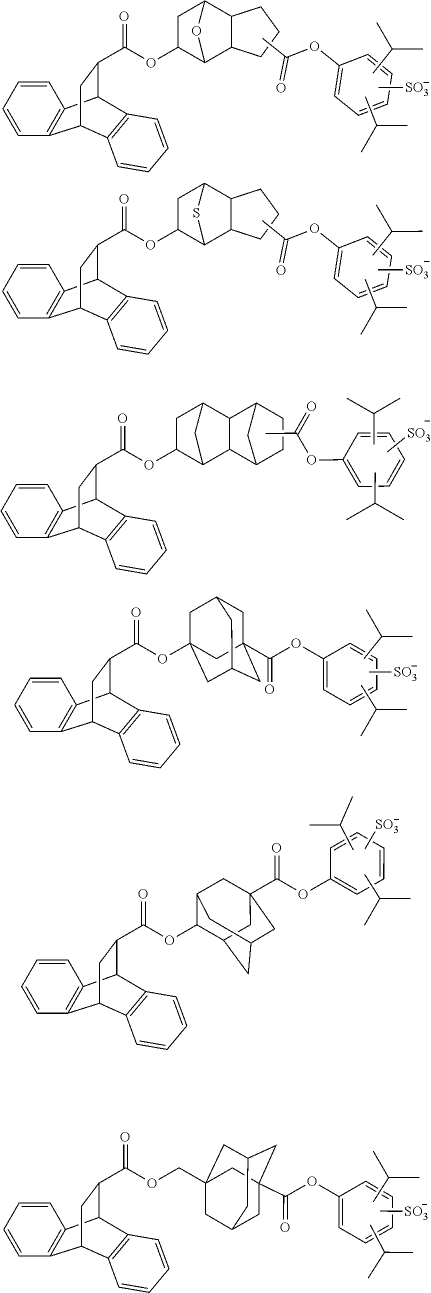

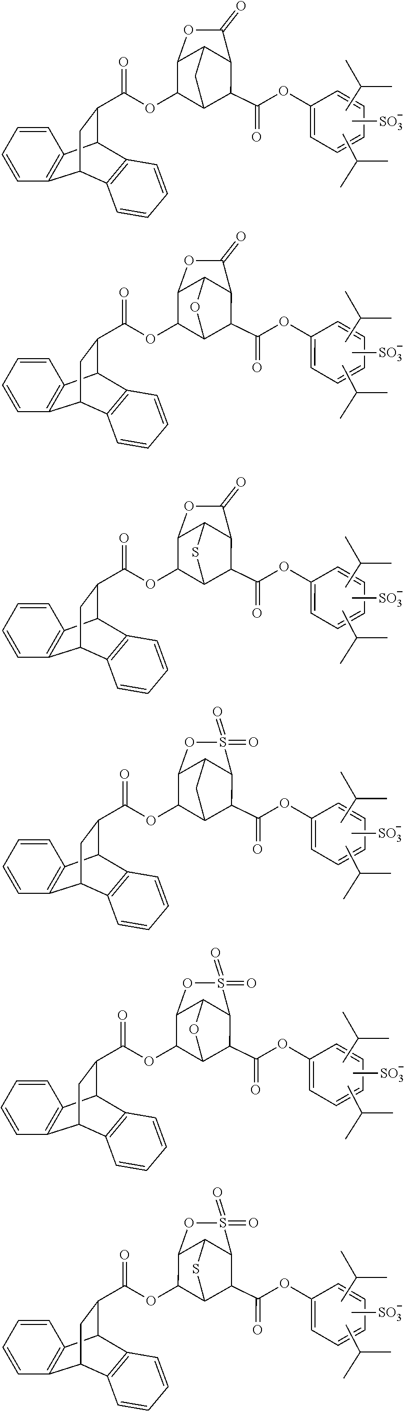

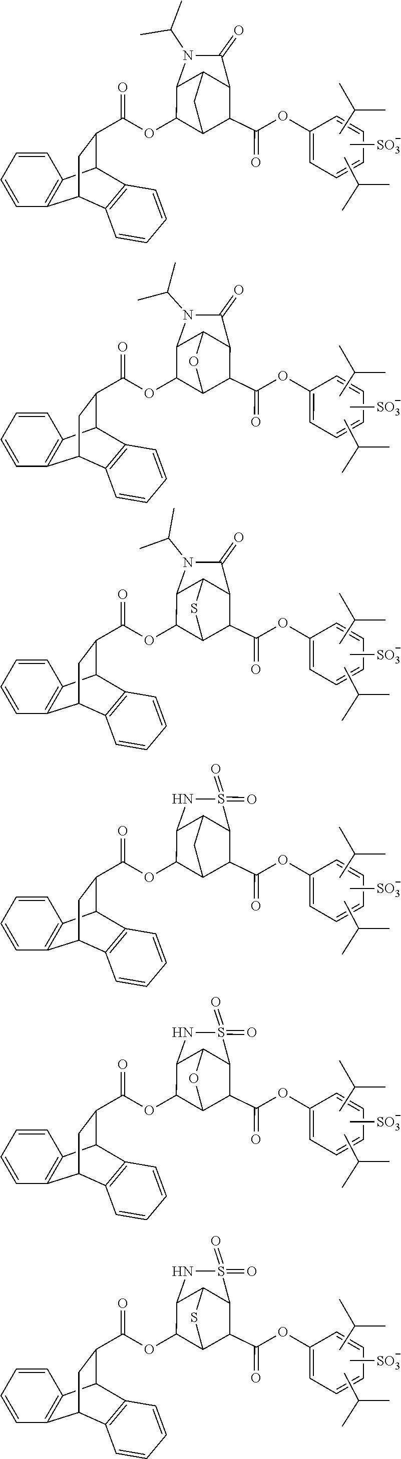

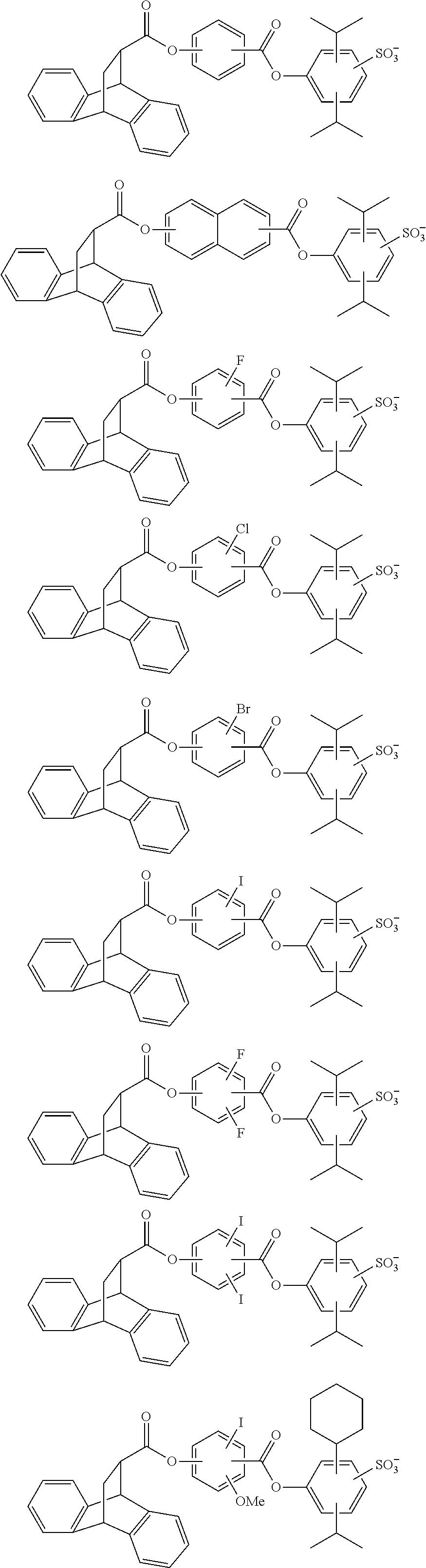

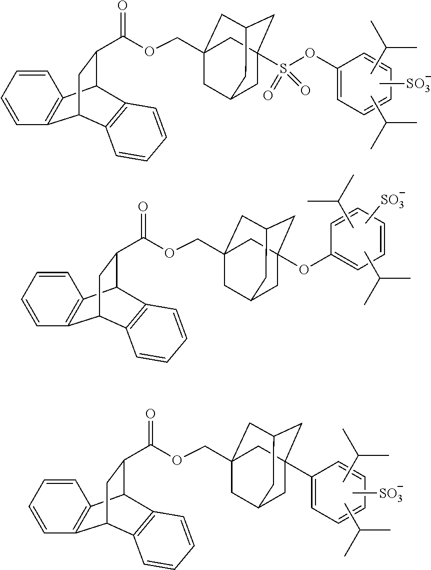

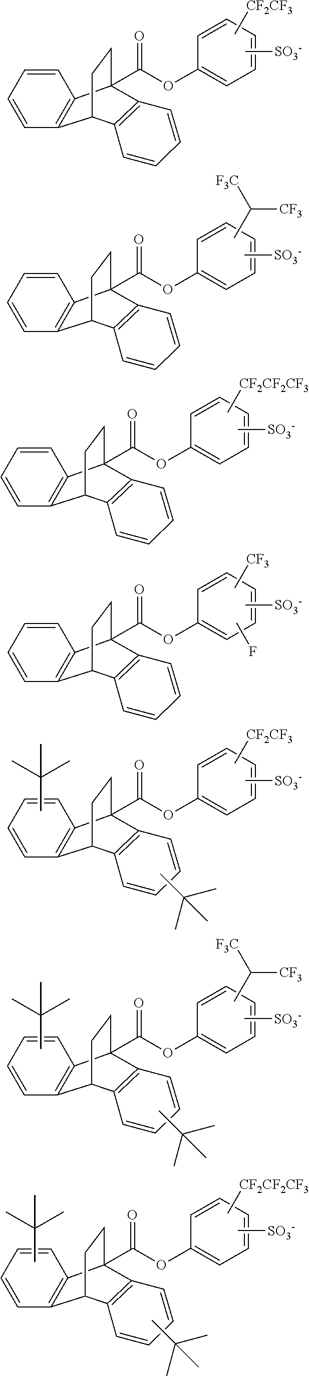

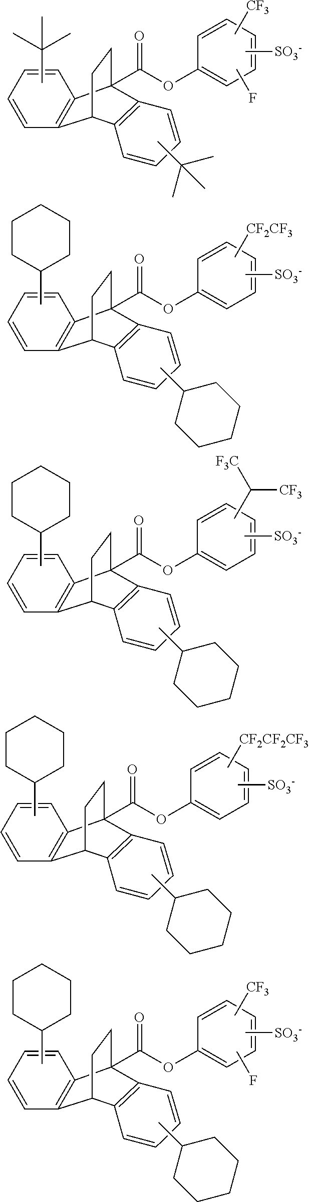

The cation contained in the onium salt of the present invention is represented by formula (1B), and such a cation can be synthesized according to, for example, the method described in Non Patent Document 1:

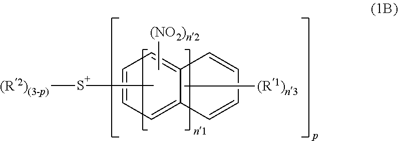

wherein “p” is 1, 2, or 3, “n′1” is 0 or 1, “n′2” is 1 or 2, and “n′3” is an integer of 0 to 6, provided that when “n′1” is 0, 1≤n′2+n′3≤5, and when “n′1” is 1, 1≤n′2+n′3≤7;
R′¹ is a halogen atom, a cyano group, a hydroxy group, a carboxy group, an alkoxycarbonyl group, a hydrocarbyl group having 1 to 20 carbon atoms and optionally comprising a heteroatom, a hydrocarbyloxy group having 1 to 20 carbon atoms and optionally comprising a heteroatom, or a hydrocarbylthio group having 1 to 20 carbon atoms and optionally comprising a heteroatom, wherein when “n′3” is 2 to 6, R′¹ are the same as or different from each other, and two R′¹ are optionally bonded to each other to form a ring together with carbon atoms to which the R′¹ are bonded; and
R′² is a halogen atom or a hydrocarbyl group having 1 to 30 carbon atoms and optionally comprising a heteroatom, wherein when “p” is 1, two R′² are the same as or different from each other, and two of three substituents bonded to S⁺ are optionally bonded to each other to form a ring together with a sulfur atom to which the substituents are bonded.

In formula (1B), “p” is 1, 2, or 3.

In formula (1B), “n′1” is 0 or 1. The onium salt has a benzene ring when “n′1” is 0, and a naphthalene ring when “n′1” is 1. From the viewpoint of solvent solubility, a benzene ring with “n′1” of 0 is preferable. “n′2” is 1 or 2. From the viewpoint of raw material procurement, “n′2” is preferably 1. “n′3” is 0 to 6. From the viewpoint of raw material procurement, “n′3” is preferably 0, 1, or 2. However, when “n′1” is 0, 1≤n′2+n′3≤5, and when “n′1” is 1, 1≤n′2+n′3≤7.

In formula (1B), R′¹ is a halogen atom, a cyano group, a hydroxy group, a carboxy group, an alkoxycarbonyl group, a hydrocarbyl group having 1 to 20 carbon atoms and optionally comprising a heteroatom, a hydrocarbyloxy group having 1 to 20 carbon atoms and optionally comprising a heteroatom, or a hydrocarbylthio group having 1 to 20 carbon atoms and optionally comprising a heteroatom. Examples of the halogen atom include a fluorine atom, a chlorine atom, a bromine atom, and an iodine atom. The hydrocarbyl group and hydrocarbyl moieties of the hydrocarbyloxy group and hydrocarbylthio group may be either saturated or unsaturated and linear, branched, or cyclic. Specific examples thereof include alkyl groups having 1 to 20 carbon atoms, such as a methyl group, an ethyl group, an n-propyl group, an isopropyl group, an n-butyl group, an isobutyl group, a sec-butyl group, a tert-butyl group, an n-pentyl group, an n-hexyl group, an n-octyl group, an n-nonyl group, an n-decyl group, an undecyl group, a dodecyl group, a tridecyl group, a tetradecyl group, a pentadecyl group, a heptadecyl group, an octadecyl group, a nonadecyl group, and an icosyl group; cyclic saturated hydrocarbyl groups having 3 to 20 carbon atoms, such as a cyclopropyl group, a cyclopentyl group, a cyclohexyl group, a cyclopropylmethyl group, a 4-methylcyclohexyl group, a cyclohexylmethyl group, a norbornyl group, and an adamantyl group; alkenyl groups having 2 to 20 carbon atoms, such as a vinyl group, a 1-propenyl group, a 2-propenyl group, a butenyl group, and a hexenyl group; cyclic unsaturated hydrocarbyl groups having 3 to 20 carbon atoms, such as a cyclohexenyl group; aryl groups having 6 to 20 carbon atoms, such as a phenyl group or a naphthyl group; aralkyl groups having 7 to 20 carbon atoms, such as a benzyl group, a 1-phenylethyl group, and a 2-phenylethyl group; and groups obtained by combining these groups. Of these, preferred is an aryl group. Some or all of the hydrogen atoms in the hydrocarbyl group are optionally substituted with a group containing a heteroatom such as an oxygen atom, a sulfur atom, a nitrogen atom, or a halogen atom, and some of —CH₂— in the hydrocarbyl group are optionally substituted with a group containing a heteroatom such as an oxygen atom, a sulfur atom, or a nitrogen atom. As a result, the hydrocarbyl group may contain, for instance, a hydroxy group, a cyano group, a fluorine atom, a chlorine atom, a bromine atom, an iodine atom, a carbonyl group, an ether bond, an ester bond, a sulfonate ester bond, a carbonate bond, a lactone ring, a sultone ring, a carboxylic anhydride (—C(═O)—O—C(═O)—), and a haloalkyl group. When “n′3” is 2 to 6, R′¹ are the same as or different from each other, and when “n′3” is 2 to 6, a plurality of R¹ are optionally bonded to each other to form a ring together with carbon atoms to which the R′¹ are bonded. The ring is preferably a 5-to 8-membered ring.

In formula (1B), R′² is a halogen atom or a hydrocarbyl group having 1 to 30 carbon atoms and optionally comprising a heteroatom, wherein when “p” is 1, two R′² are the same as or different from each other.

Specific examples of the halogen atom represented by R′² include a fluorine atom, a chlorine atom, a bromine atom, and an iodine atom.

The hydrocarbyl group represented by R′² may be either saturated or unsaturated and linear, branched, or cyclic. Specific examples thereof include alkyl groups having 1 to 30 carbon atoms, such as a methyl group, an ethyl group, an n-propyl group, an isopropyl group, an n-butyl group, an isobutyl group, a sec-butyl group, and a tert-butyl group; cyclic saturated hydrocarbyl groups having 3 to 30 carbon atoms, such as a cyclopropyl group, a cyclopentyl group, a cyclohexyl group, a cyclopropylmethyl group, a 4-methylcyclohexyl group, a cyclohexylmethyl group, a norbornyl group, and an adamantyl group; alkenyl groups having 2 to 30 carbon atoms, such as a vinyl group, a 1-propenyl group, a 2-propenyl group, a butenyl group, and a hexenyl group; cyclic unsaturated hydrocarbyl groups having 3 to 30 carbon atoms, such as a cyclohexenyl group; aryl groups having 6 to 30 carbon atoms, such as a phenyl group, a naphthyl group, and a thienyl group; aralkyl groups having 7 to 30 carbon atoms, such as a benzyl group, a 1-phenylethyl group, and a 2-phenylethyl group; and groups obtained by combining these groups, but the aryl group is preferable. Some or all of the hydrogen atoms in the hydrocarbyl group are optionally substituted with a group containing a heteroatom such as an oxygen atom, a sulfur atom, a nitrogen atom, or a halogen atom, and some of —CH₂— in the hydrocarbyl group are optionally substituted with a group containing a heteroatom such as an oxygen atom, a sulfur atom, or a nitrogen atom. As a result, the hydrocarbyl group may contain, for instance, a hydroxy group, a fluorine atom, a chlorine atom, a bromine atom, an iodine atom, a cyano group, a nitro group, a carbonyl group, an ether bond, an ester bond, a sulfonate ester bond, a carbonate bond, a lactone ring, a sultone ring, a carboxylic anhydride (—C(═O)—O—C(═O)—), and a haloalkyl group.

In addition, two of three substituents bonded to S⁺ are optionally bonded to each other to form a ring together with a sulfur atom to which the substituents are bonded. At this point, specific examples of the structure of the ring include those represented by formulae below:

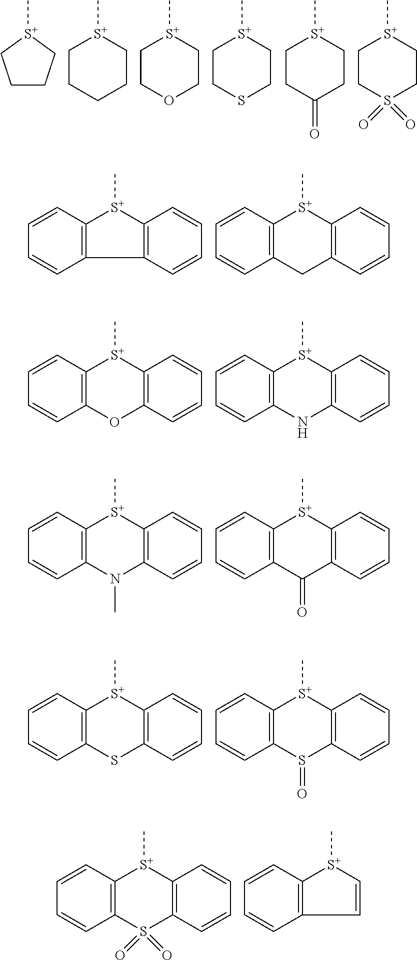

wherein the dashed line represents a bond.

The cation represented by formula (1B) is preferably represented by formula (1B-1):

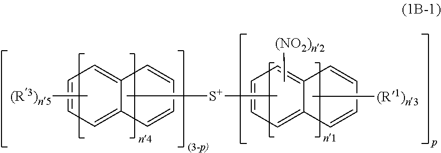

wherein “p”, “n′1” to “n′3”, and R¹ are the same as above;
“n′4” is 0 or 1, “n′5” is an integer of 0 to 5; and
R′³ is a halogen atom, a cyano group, a hydroxy group, a carboxy group, an alkoxycarbonyl group, a hydrocarbyl group having 1 to 20 carbon atoms and optionally comprising a heteroatom, a hydrocarbyloxy group having 1 to 20 carbon atoms and optionally comprising a heteroatom, or a hydrocarbylthio group having 1 to 20 carbon atoms and optionally comprising a heteroatom, wherein when “n′5” is 2 to 5, R′³ are the same as or different from each other, and two R′³ are optionally bonded to each other to form a ring together with carbon atoms to which the R′³ are bonded.

In formula (1B-1), “n′4” is 0 or 1. The onium salt has a benzene ring when “n′4” is 0, and a naphthalene ring when “n′4” is 1. From the viewpoint of solvent solubility, a benzene ring with “n′4” of 0 is preferable. “n′5” is 0, 1, 2, 3, 4, or 5. From the viewpoint of raw material procurement, “n′5” is preferably 0, 1, or 2.

In formula (1B-1), R′³ is a halogen atom, a cyano group, a hydroxy group, a carboxy group, an alkoxycarbonyl group, a hydrocarbyl group having 1 to 20 carbon atoms and optionally comprising a heteroatom, a hydrocarbyloxy group having 1 to 20 carbon atoms and optionally comprising a heteroatom, or a hydrocarbylthio group having 1 to 20 carbon atoms and optionally comprising a heteroatom. The hydrocarbyl group and hydrocarbyl moieties of the hydrocarbyloxy group and hydrocarbylthio group may be either saturated or unsaturated and linear, branched, or cyclic. Specific examples thereof include, but are not limited to, the same hydrocarbyl group represented by R′¹. When “n′5” is 2 to 5, R′³ are the same as or different from each other, and two R′³ are optionally bonded to each other to form a ring together with carbon atoms to which the R′³ are bonded.

Examples of the cation in the sulfonium salt represented by formula (1B) are given below, but not limited thereto.

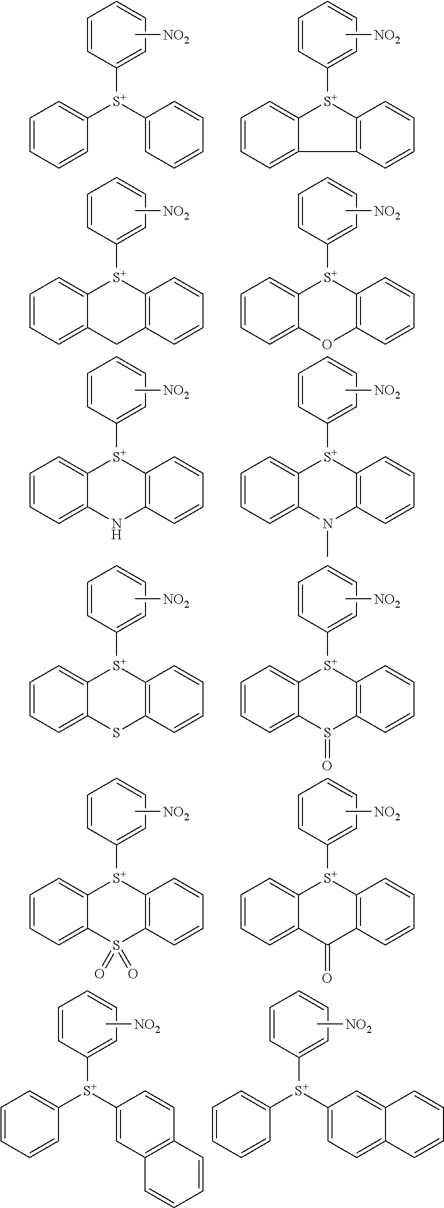

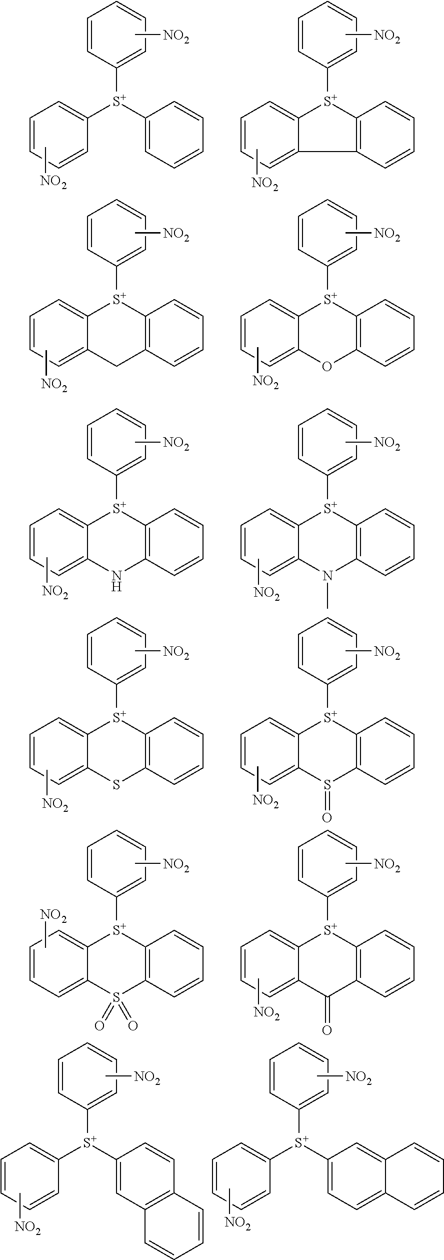

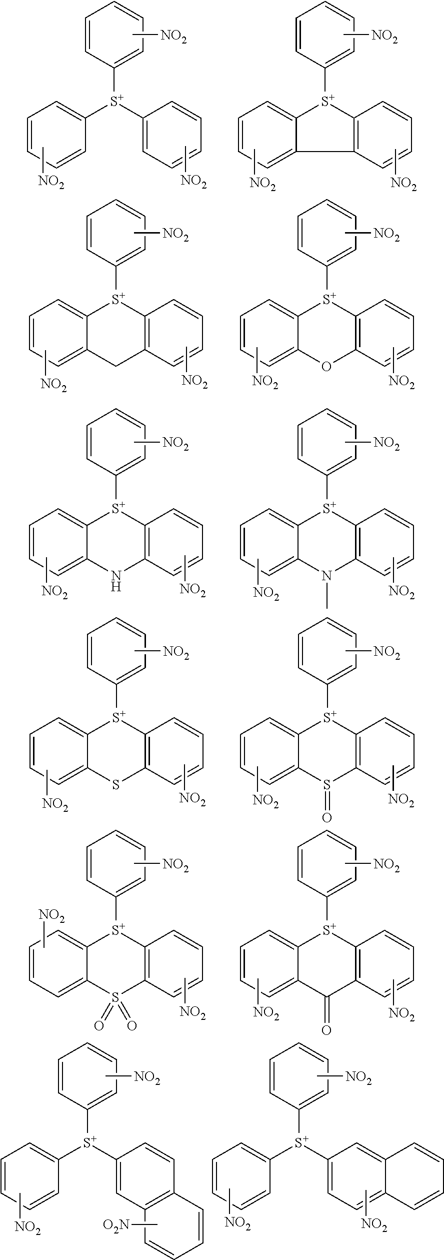

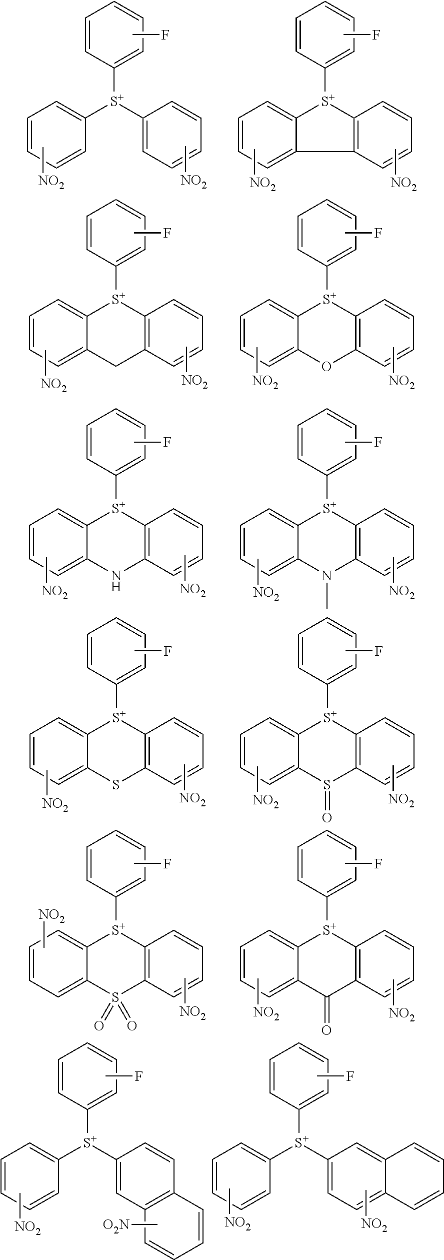

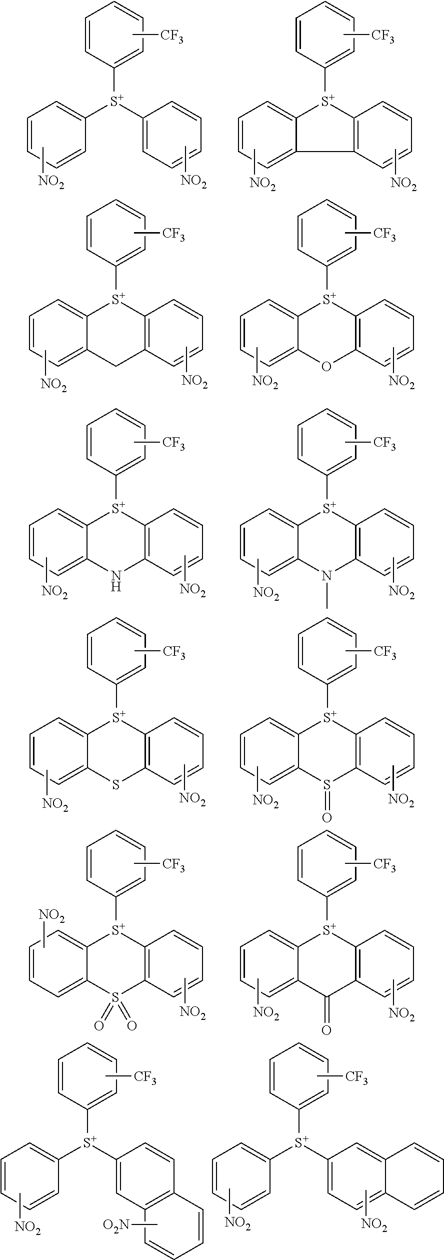

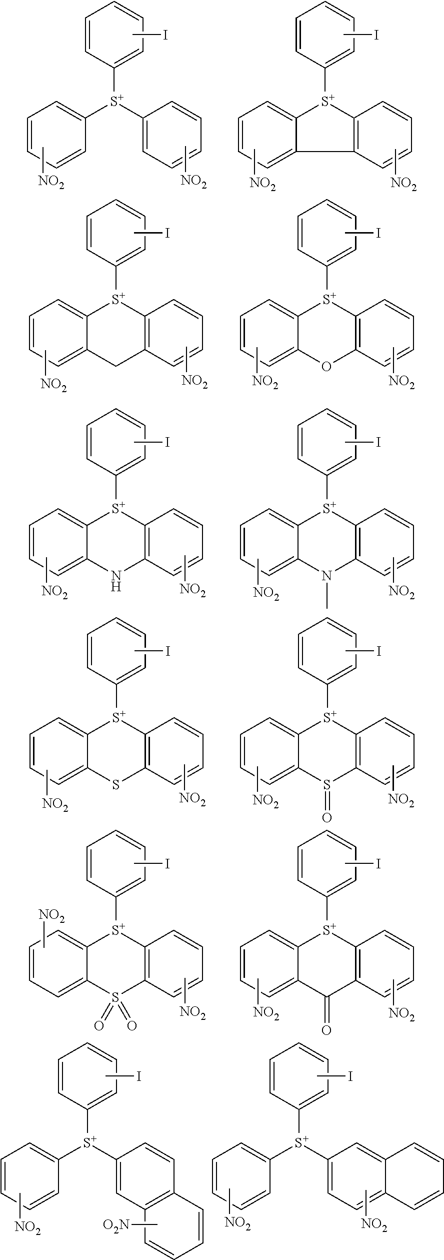

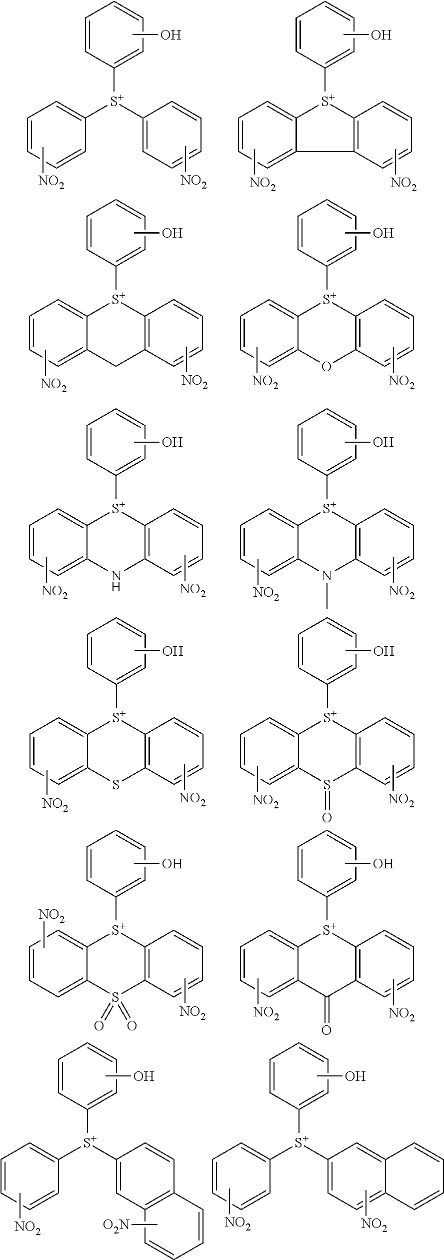

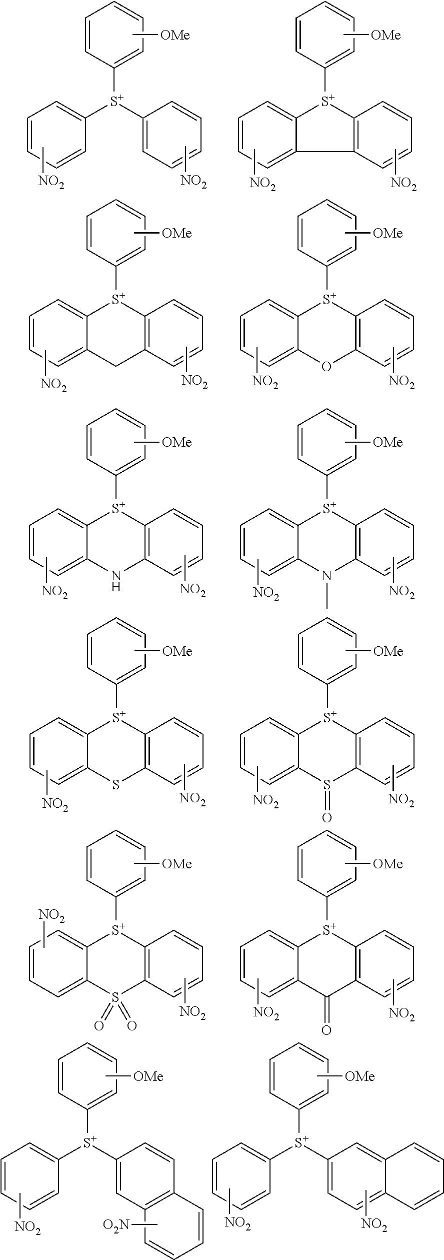

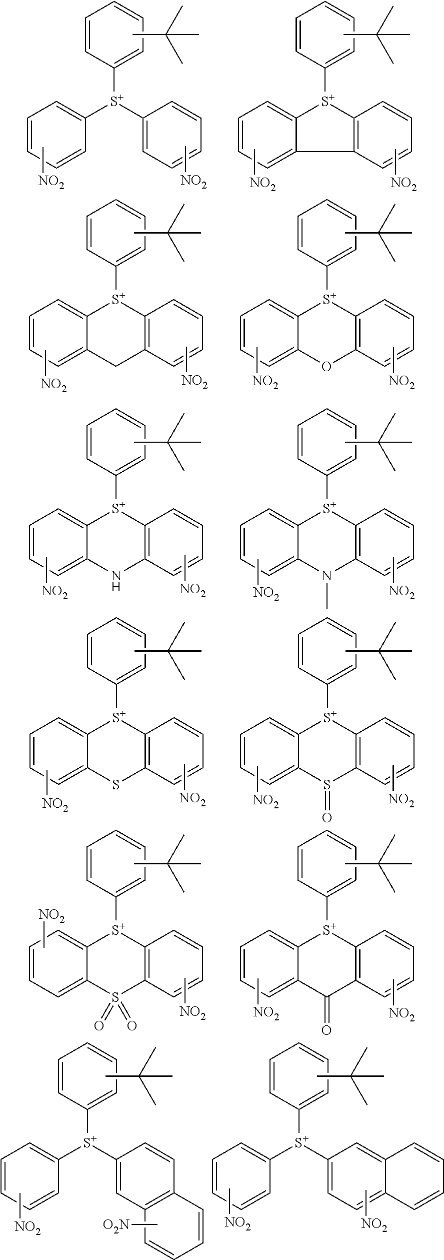

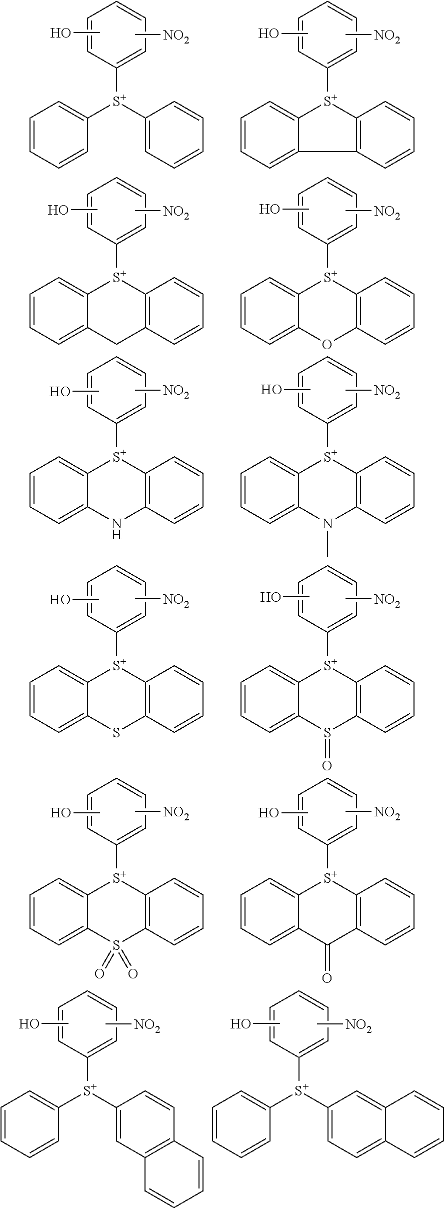

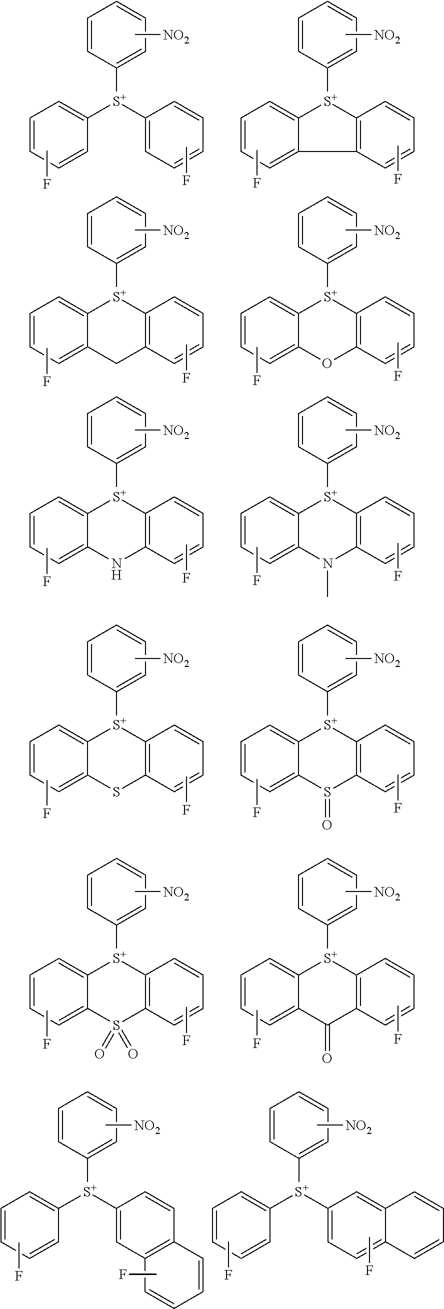

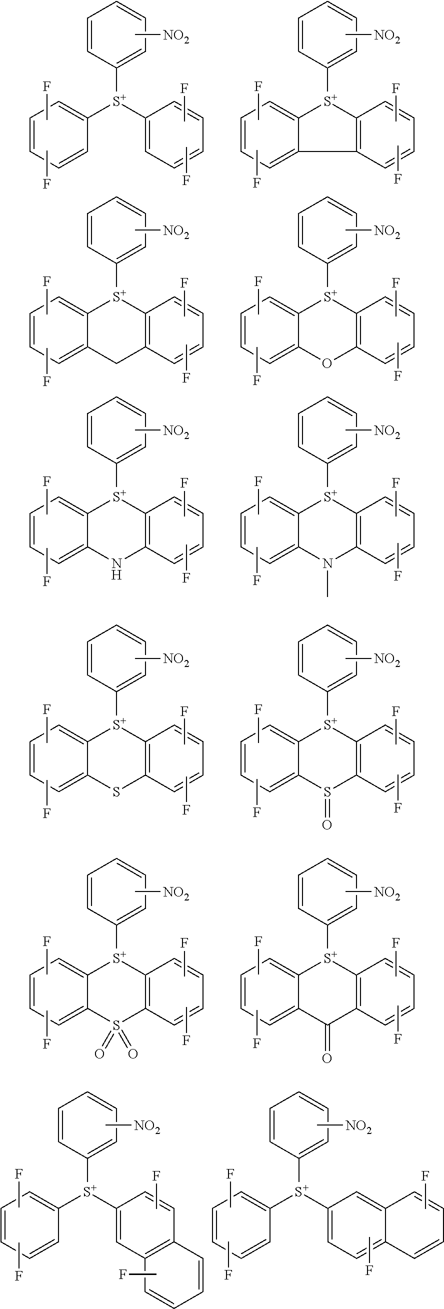

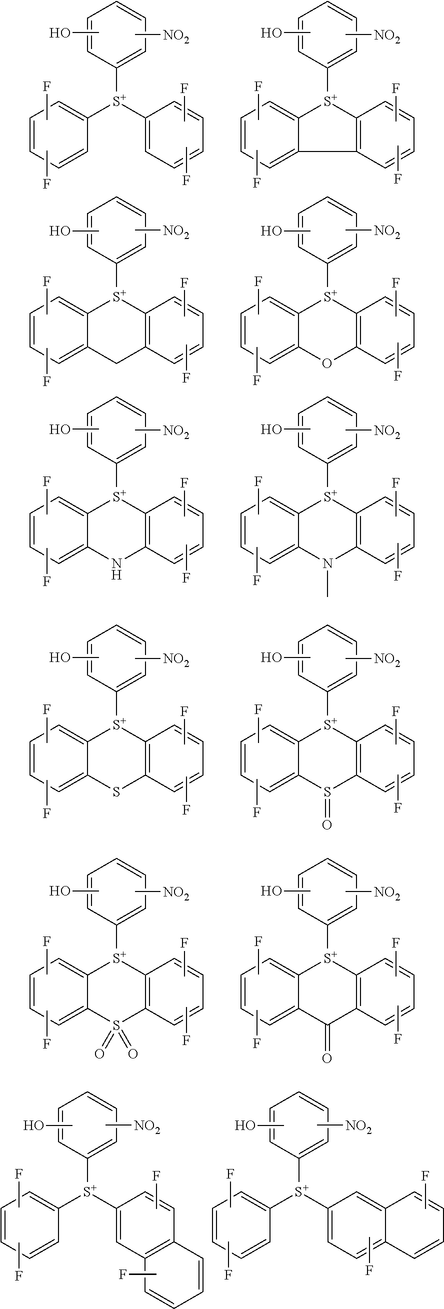

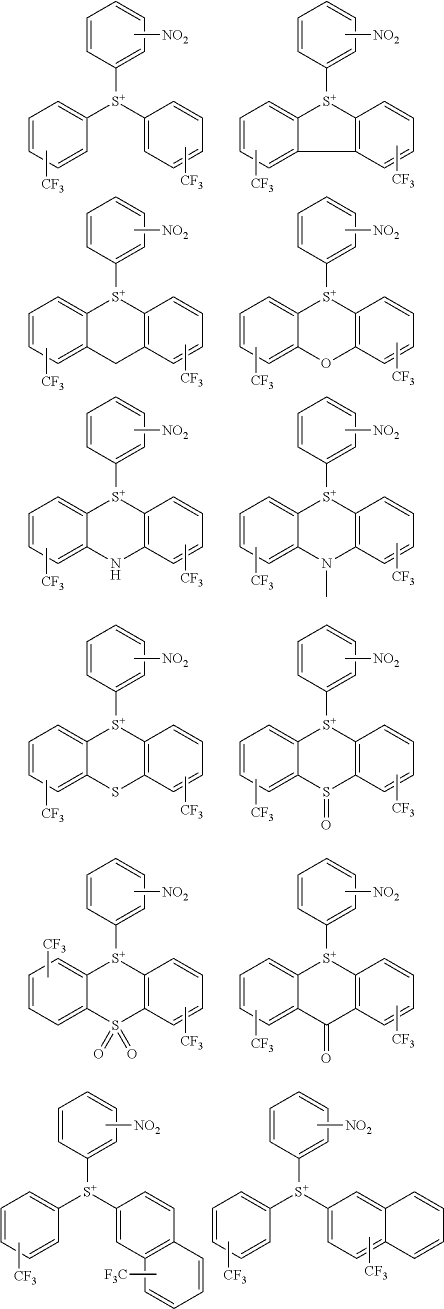

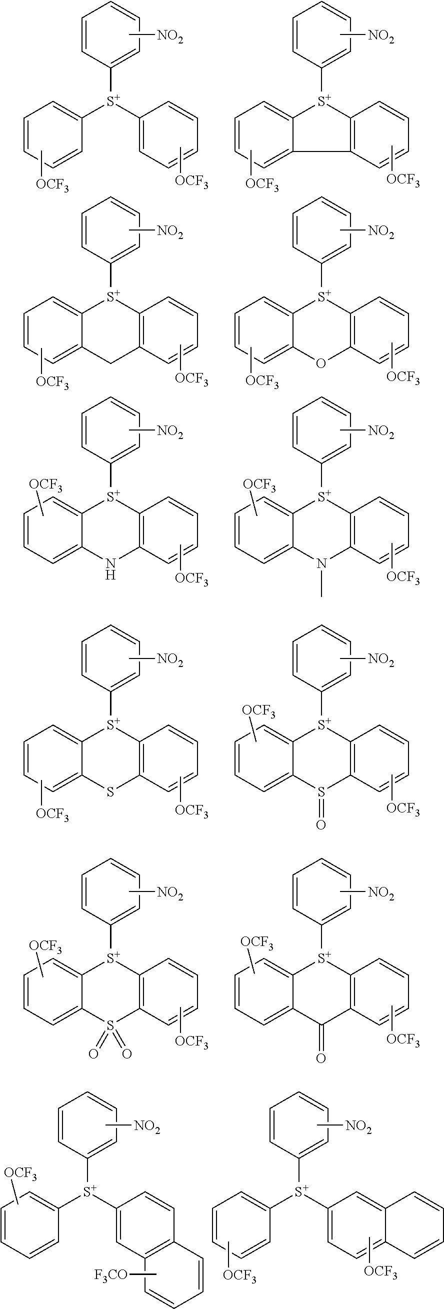

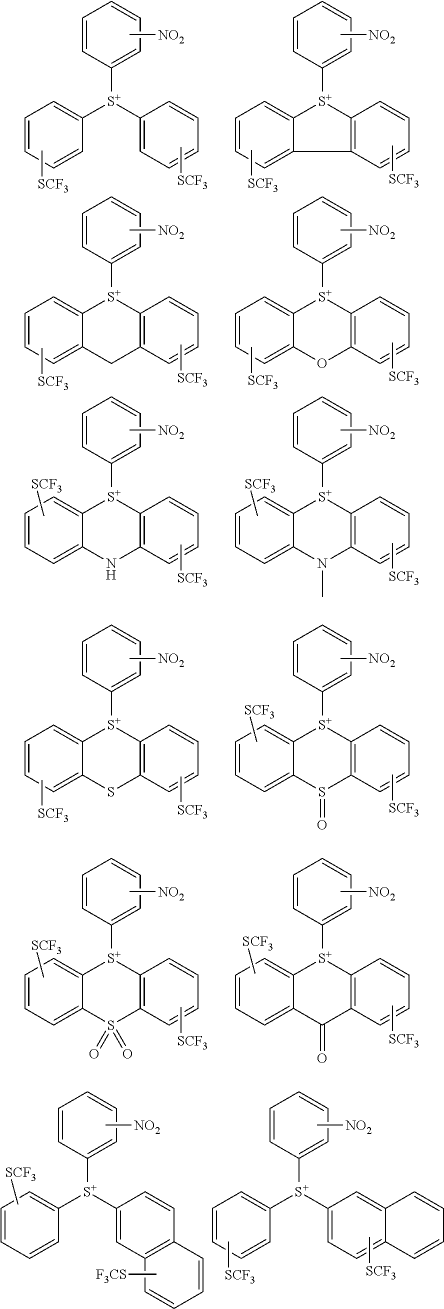

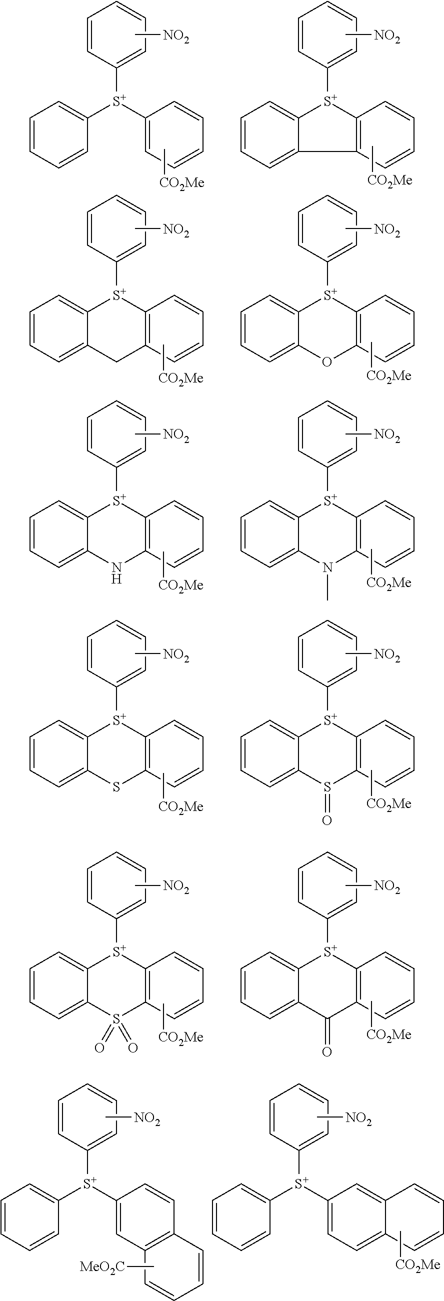

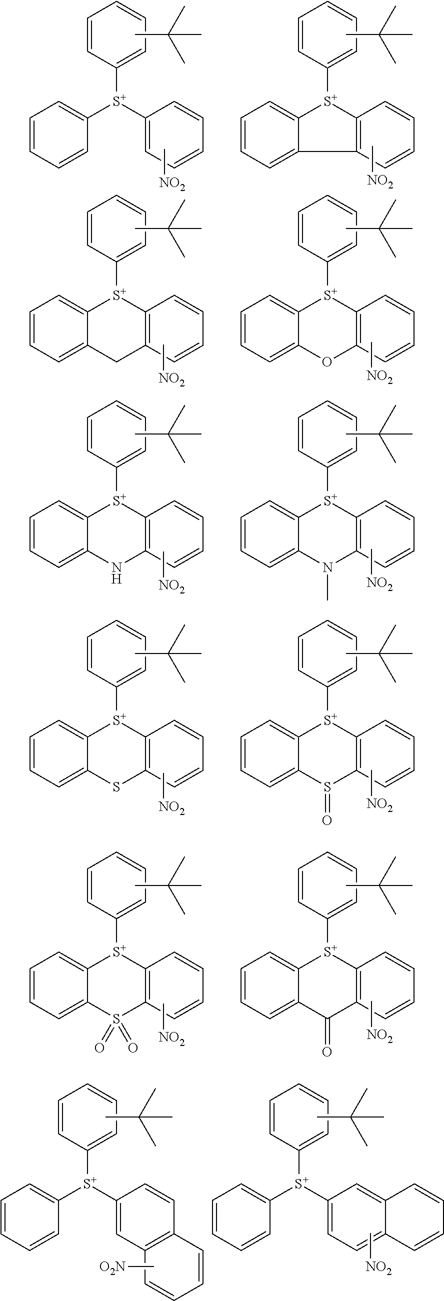

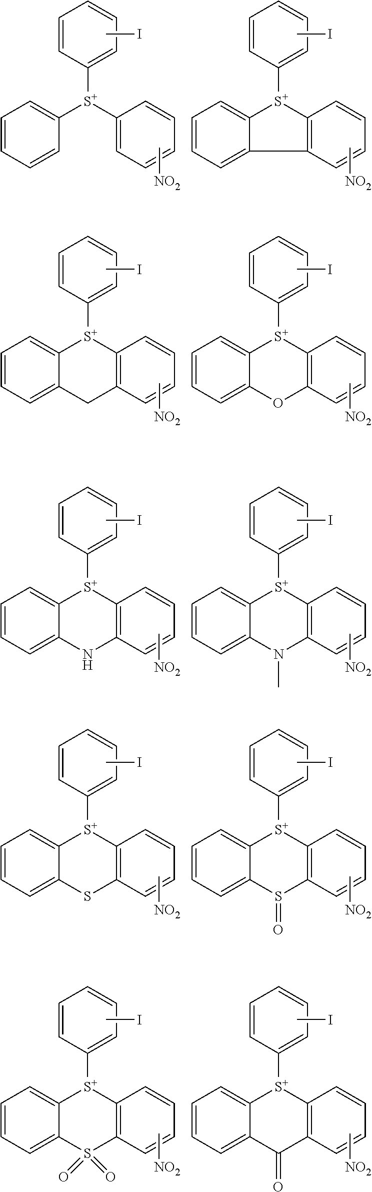

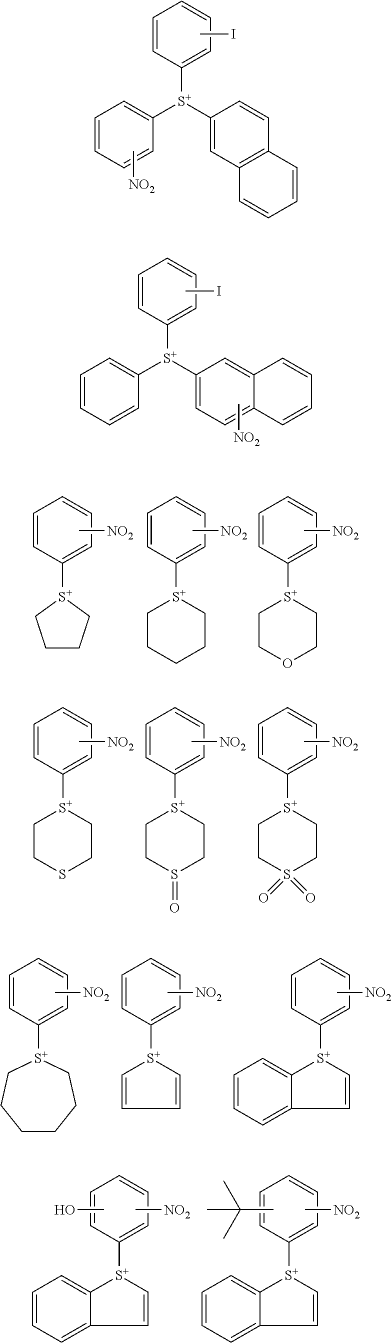

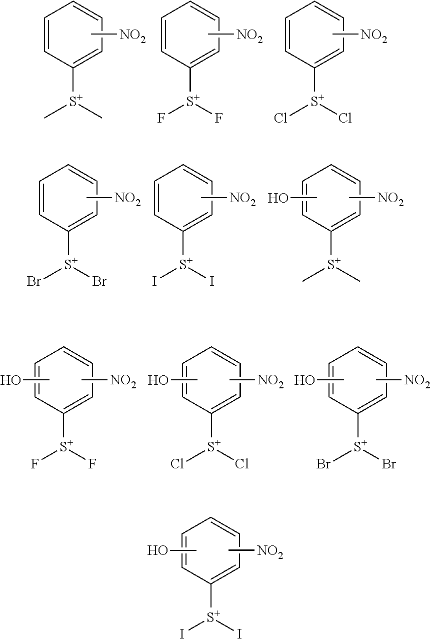

Specific examples of the onium salt of the present invention include any combination of the anions and cations described above.

Examples of the method for synthesizing the onium salt of the present invention include the methods described in, for instance, JP 2010-155824 A and JP 7067271 B, but the aforementioned production method is an example, and the method for producing the onium salt of the present invention is not limited thereto.

The onium salt of the present invention is structurally characterized by having an aromatic sulfonate anion substituted with a hydrocarbyl group containing at least one aromatic ring and a sulfonium cation containing a nitro group. It is known that secondary electrons are emitted when the base polymer is irradiated with EUV light, but the nitro group of the sulfonium cation lowers the energy level of the lowest unoccupied molecular orbital (LUMO) in the frontier orbital theory due to the electron withdrawing effect, making it easier to receive the generated secondary electrons, thereby promoting the decomposition of the cation and effectively generating an acid. Since the nitro group has a resonance structure in which the nitrogen atom of the nitro group is positively charged and one oxygen atom thereof is negatively charged, the nitro group can be expected to function as an acid diffusion suppressing group by interacting with a proton of a generated acid at the negative charge on the oxygen atom. Furthermore, the affinity with an alkali developing solution is high due to the charge, and the risk of developing defects in the exposed region is also reduced since the residue after cation decomposition is efficiently removed with respect to the alkali developing solution. On the other hand, the hydrocarbyl group containing at least one aromatic ring has a large excluded volume, acts as a bulky substituent, and highly suppresses the diffusion of the generated acid. In particular, such an effect is easily obtained when the hydrocarbyl group containing at least one aromatic ring has an aromatic ring structure having a substituent represented by formula (W-1) or a fused ring structure having a substituent represented by formula (W-2). The hydrocarbyl group has resistance to an alkaline developing solution to reduce film loss of a pattern of an unexposed region. Meanwhile, the aromatic sulfonic acid structure includes a rigid structure of the generated acid and exhibits an effect of suppressing acid diffusion. The aromatic ring forming the aromatic sulfonic acid structure preferably has a fluorine atom or an electron-withdrawing sulfonate ester bond as a linking group, whereby the acidity of the generated acid can be enhanced to efficiently deprotect the acid labile group of the base polymer. In addition, since a fluorine atom is an element having a high absorption effect of EUV light although not as high as an iodine atom, an increase in the number of fluorine atoms increases the amount of secondary electrons generated, promotes the decomposition of cations, and contributes to higher sensitivity. JP 7109178 B describes an alkanesulfonic acid-type photoacid generator having 2 to 4 fluorine atoms, but there is a concern about development defects due to relatively large acid diffusion and poor solvent solubility because the alkanesulfonic acid-type photoacid generator is alkanesulfonic acid. By virtue of these synergistic effects, the resist composition containing the onium salt of the present invention has high sensitivity and low acid diffusivity, making it possible to form a pattern excellent in LWR of a line pattern and CDU of a hole pattern and resistant to pattern collapse, and is therefore suitable for forming a fine pattern.

The onium salt can be suitably used as a photoacid generator.

### Chemically Amplified Resist Composition (A) Photoacid Generator

A chemically amplified resist composition of the present invention contains a photoacid generator composed of the onium salt as an essential component.

In the chemically amplified resist composition of the present invention, the content of the photoacid generator composed of an onium salt as the component (A) is preferably 0.1 to 40 parts by mass, and more preferably 0.5 to 30 parts by mass, based on 80 parts by mass of a base polymer described later. A content of the component (A) within the range is preferable because of good sensitivity and resolution without the risk of foreign matter being formed after development or during stripping of the resist film. The photoacid generator(A) may be used singly or in combination with two or more thereof.

### (B) Base Polymer

The chemically amplified resist composition of the present invention may contain a base polymer as a component (B). The base polymer (B) may further contain a repeating unit represented by formula (a1) (hereinafter referred to as repeating unit a1) or a repeating unit represented by formula (a2) (hereinafter referred to as repeating unit a2):

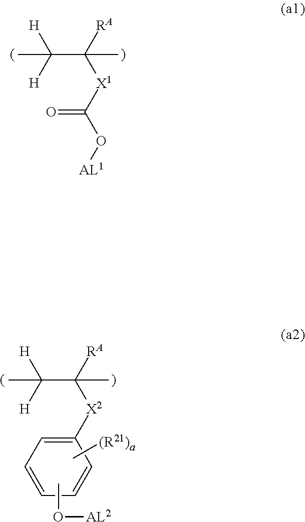

wherein each R^{A} is independently a hydrogen atom, a fluorine atom, a methyl group, or a trifluoromethyl group;
X¹ is a single bond, a phenylene group, a naphthylene group, or *—C(═O)—O—X¹¹—, wherein the phenylene group or naphthylene group is optionally substituted with a hydroxy group, a nitro group, a cyano group, a saturated hydrocarbyl group having 1 to 10 carbon atoms and optionally comprising a fluorine atom, a saturated hydrocarbyloxy group having 1 to 10 carbon atoms and optionally comprising a fluorine atom, or a halogen atom; X¹¹ is a saturated hydrocarbylene group having 1 to 10 carbon atoms, a phenylene group, or a naphthylene group, wherein the saturated hydrocarbylene group optionally comprises a hydroxy group, an ether bond, an ester bond, or a lactone ring;
X² is a single bond or *—C(═O)—O—;
“*” represents a bond to a carbon atom of a main chain;
R²¹ is a halogen atom, a cyano group, a hydroxy group, a nitro group, a hydrocarbyl group having 1 to 20 carbon atoms and optionally comprising a heteroatom, a hydrocarbyloxy group having 1 to 20 carbon atoms and optionally comprising a heteroatom, a hydrocarbylcarbonyl group having 2 to 20 carbon atoms and optionally comprising a heteroatom, a hydrocarbylcarbonyloxy group having 2 to 20 carbon atoms and optionally comprising a heteroatom, or a hydrocarbyloxycarbonyl group having 2 to 20 carbon atoms and optionally comprising a heteroatom;
A^{L1} and A^{L2} are each independently an acid labile group; and
“a” is an integer of 0 to 4.

In formulae (a1) and (a2), each R^{A} is independently a hydrogen atom, a fluorine atom, a methyl group, or a trifluoromethyl group.

In formula (a1), X¹ is a single bond, a phenylene group, a naphthylene group, or *—C(═O)—O—X¹¹—, wherein the phenylene group or naphthylene group is optionally substituted with a hydroxy group, a nitro group, a cyano group, a saturated hydrocarbyl group having 1 to 10 carbon atoms and optionally comprising a fluorine atom, a saturated hydrocarbyloxy group having 1 to 10 carbon atoms and optionally comprising a fluorine atom, or a halogen atom; X¹¹ is a saturated hydrocarbylene group having 1 to 10 carbon atoms, a phenylene group, or a naphthylene group, wherein the saturated hydrocarbylene group optionally comprises a hydroxy group, an ether bond, an ester bond, or a lactone ring; and “*” represents a bond to a carbon atom of a main chain.

In formula (a2), X² is a single bond or *—C(═O)—O—, and “*” represents a bond to a carbon atom of a main chain; R²¹ is a halogen atom, a cyano group, a hydroxy group, a nitro group, a hydrocarbyl group having 1 to 20 carbon atoms and optionally comprising a heteroatom, a hydrocarbyloxy group having 1 to 20 carbon atoms and optionally comprising a heteroatom, a hydrocarbylcarbonyl group having 2 to 20 carbon atoms and optionally comprising a heteroatom, a hydrocarbylcarbonyloxy group having 2 to 20 carbon atoms and optionally comprising a heteroatom, or a hydrocarbyloxycarbonyl group having 2 to 20 carbon atoms and optionally comprising a heteroatom; and “a” is an integer of 0 to 4, preferably 0 or 1.

In formulae (a1) and (a2), A^{L1} and A^{L2} are each independently an acid labile group. Specific examples of the acid labile group include groups described in JP 2013-80033 A and JP 2013-83821 A.

Typically, specific examples of the acid labile group include groups represented by formulae (AL-1) to (AL-3):

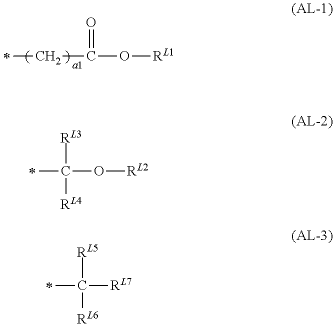

wherein the dashed line represents a bond.

In formulae (AL-1) and (AL-2), R^{L1} and R^{L2} are each independently a hydrocarbyl group having 1 to 40 carbon atoms and may contain a heteroatom such as an oxygen atom, a sulfur atom, a nitrogen atom, or a fluorine atom. The hydrocarbyl group may be either saturated or unsaturated and may be linear, branched, or cyclic. The hydrocarbyl group preferably has 1 to 20 carbon atoms.

In formula (AL-1), “a1” is an integer of 0 to 10, preferably an integer of 1 to 5.

In formula (AL-2), R^{L3} and R^{L4} are each independently a hydrogen atom or a hydrocarbyl group having 1 to 20 carbon atoms and may contain a heteroatom such as an oxygen atom, a sulfur atom, a nitrogen atom, or a fluorine atom. The hydrocarbyl group may be either saturated or unsaturated and may be linear, branched, or cyclic. The hydrocarbyl group preferably has 1 to 20 carbon atoms. In addition, any two of R^{L2}, R^{L3}, and R^{L4} are optionally bonded to each other to form a ring having 3 to 20 carbon atoms together with a carbon atom or carbon and oxygen atoms to which the two of R^{L2}, R^{L3}, and R^{L4} are bonded. The ring preferably has 4 to 16 carbon atoms and is particularly preferably an alicyclic ring.

In formula (AL-3), R^{L5}, R^{L6}, and R^{L7} are each independently a hydrocarbyl group having 1 to 20 carbon atoms and may contain a heteroatom such as an oxygen atom, a sulfur atom, a nitrogen atom, or a fluorine atom. The hydrocarbyl group may be either saturated or unsaturated and may be linear, branched, or cyclic. The hydrocarbyl group preferably has 1 to 20 carbon atoms. In addition, any two of R^{L5}, R^{L6}, and R^{L7} are optionally bonded to each other to form a ring having 3 to 20 carbon atoms together with a carbon atom to which the two of R^{L5}, R^{L6}, and R^{L7} are bonded. The ring preferably has 4 to 16 carbon atoms and is particularly preferably an alicyclic ring.

Specific examples of the repeating unit a1 are given below, but not limited thereto. In formulae below, R^{A} and AL¹ are the same as above.

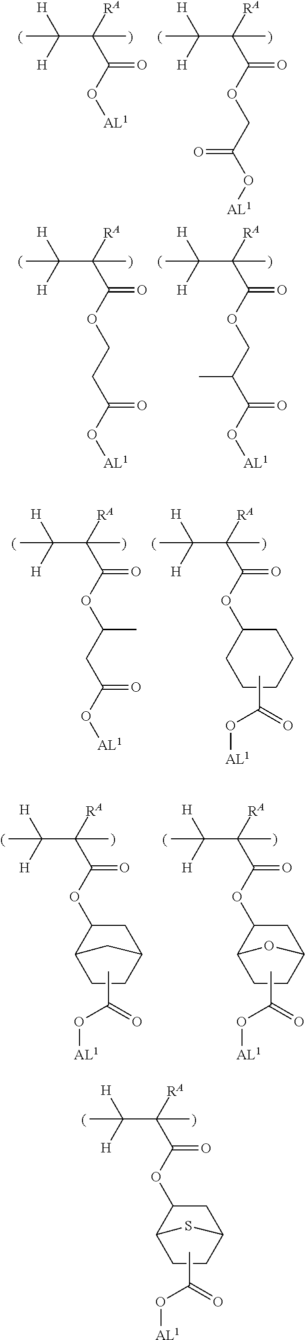

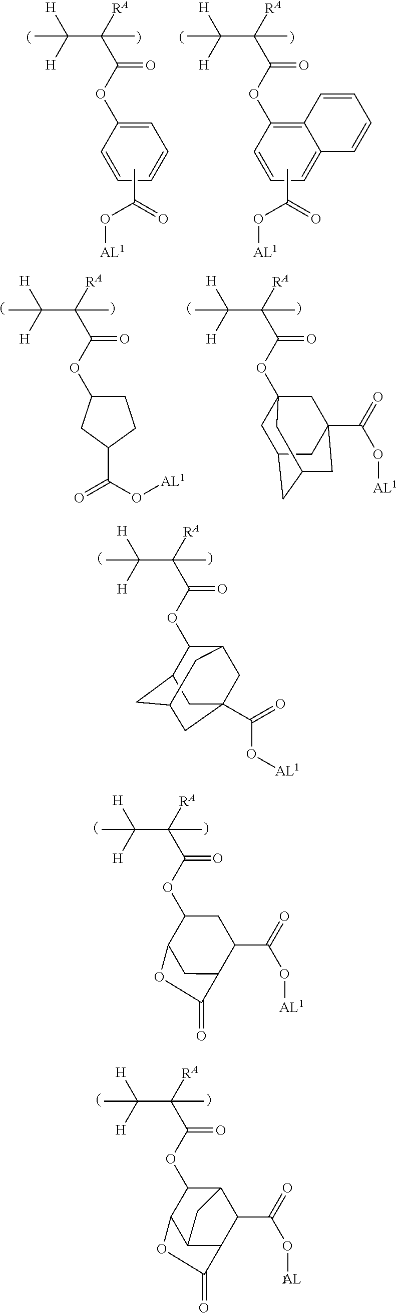

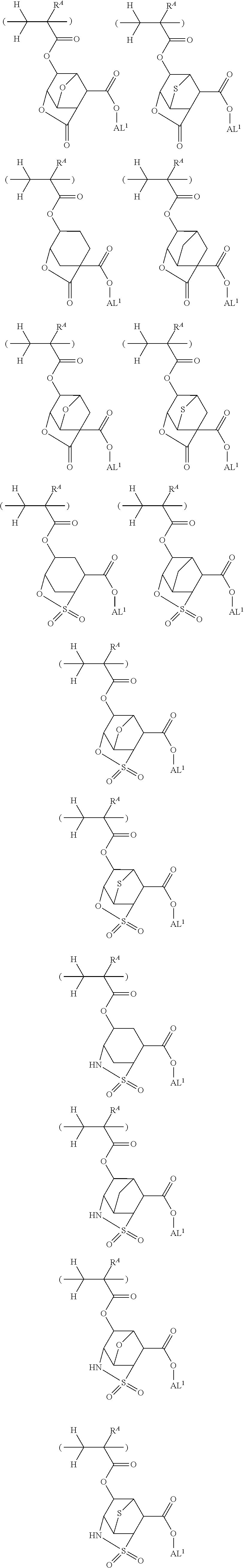

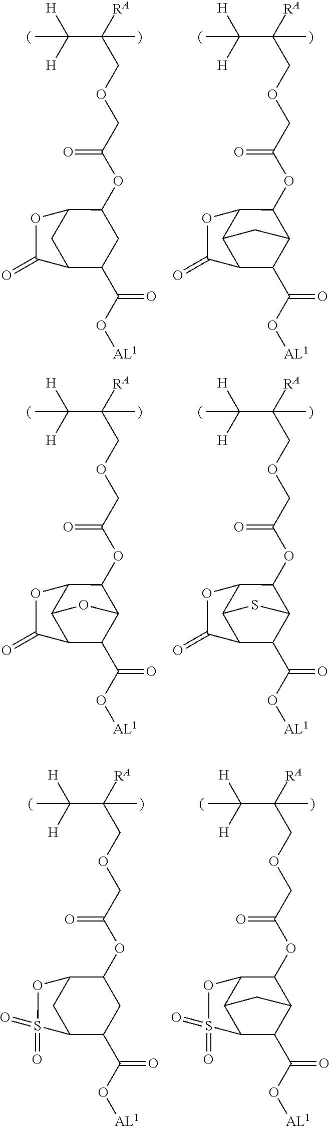

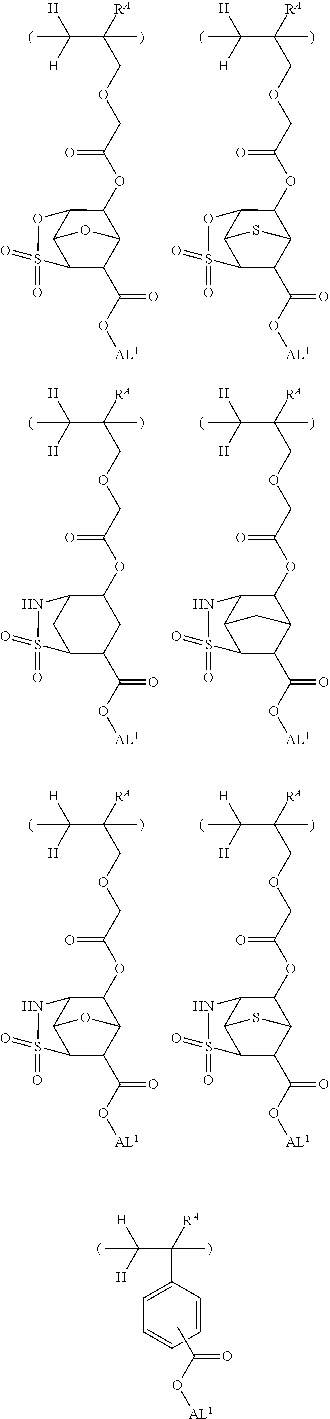

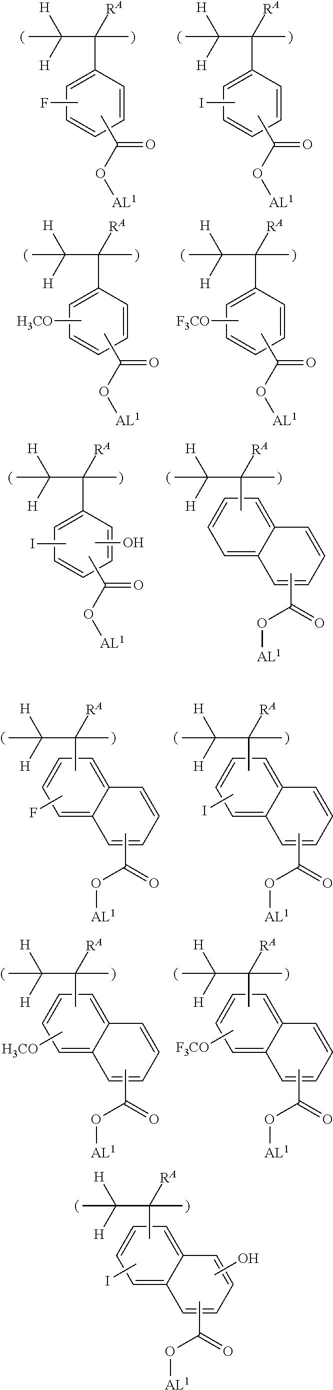

Specific examples of the repeating unit a2 are given below, but not limited thereto. In formulae below, R^{A} and AL² are the same as above.

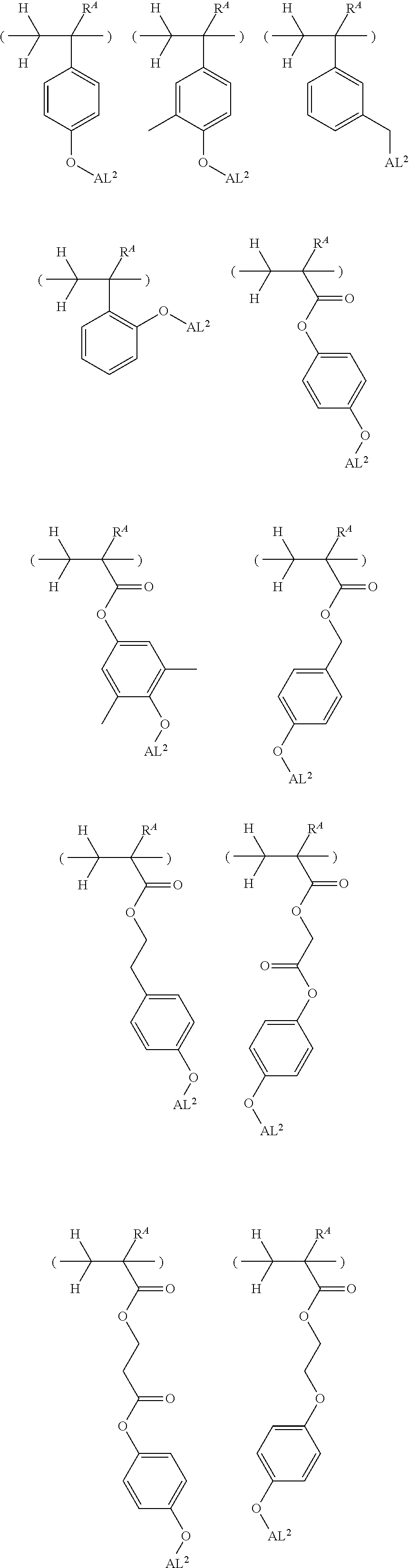

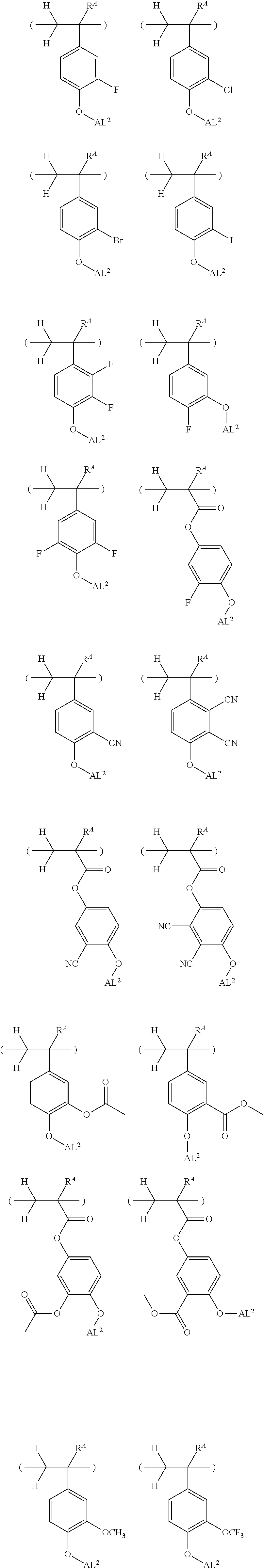

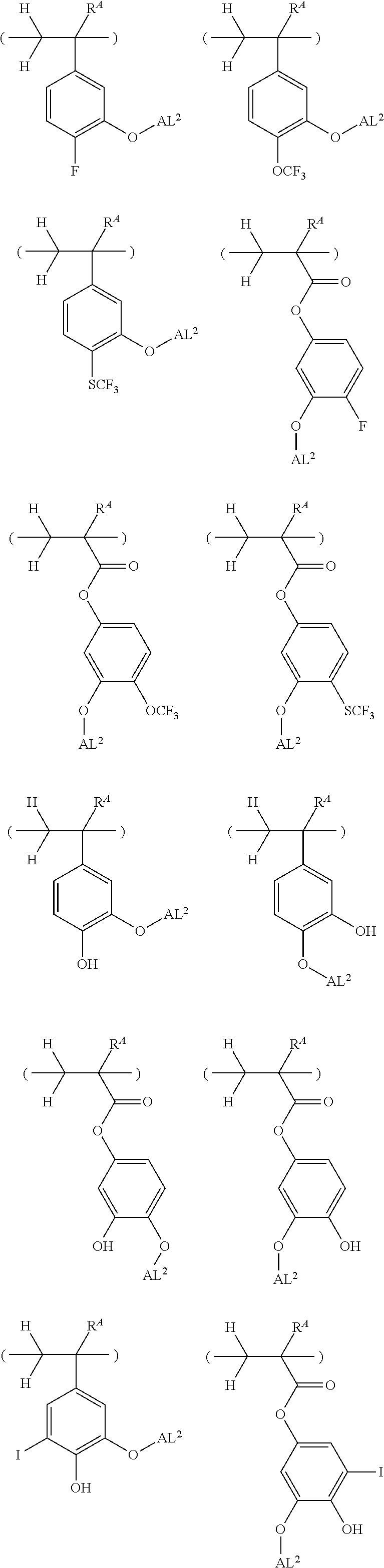

The polymer may further contain a repeating unit represented by formula (a3) (hereinafter referred to as repeating unit a3).

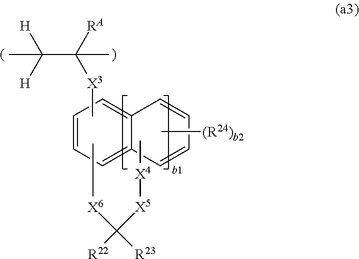

In formula (a3), “b1” is 0 or 1. The onium salt has a benzene ring when “b1” is 0, and a naphthalene ring when “b1” is 1. From the viewpoint of solvent solubility, a benzene ring with “b1” of 0 is preferable. “b2” is an integer of 0 to 3 when the “b1” is 0, and an integer of 0 to 5 when the “b1” is 1. From the viewpoint of raw material procurement, “b2” is preferably 0, 1, 2, or 3, and more preferably 0, 1, or 2.

In formula (a3), R^{A} is a hydrogen atom, a fluorine atom, a methyl group, or a trifluoromethyl group. Of these, R^{A} is preferably a hydrogen atom or a methyl group, and further preferably a hydrogen atom.

In formula (a3), X³ is a single bond, *—C(═O)—O—, or *—C(═O)—NH—, and “*” represents a bond to a carbon atom of a main chain. Of these, X³ is preferably a single bond or *—C(═O)—O—, and further preferably a single bond.

In formula (a3), X⁴ is a single bond, an aliphatic hydrocarbylene group having 1 to 4 carbon atoms, a carbonyl group, a sulfonyl group, or a group obtained by combining these groups. Of these, X⁴ is preferably a single bond, a carbonyl group, or a sulfonyl group from the viewpoint of raw material procurement, and more preferably a single bond or a carbonyl group from the viewpoint of a polar group formed after the reaction.

In formula (a3), X⁵ and X⁶ are each independently an oxygen atom or a sulfur atom, provided that X⁴ and X⁶ are bonded to adjacent carbon atoms of an aromatic ring. X⁵ and X⁶ are the same as or different from each other, and from the reactivity, both X⁵ and X⁶ are preferably oxygen atoms.

In formula (a3), R²² and R²³ are each independently a hydrogen atom or a hydrocarbyl group having 1 to 20 carbon atoms and optionally comprising a heteroatom. The hydrocarbyl group may be either saturated or unsaturated and may be linear, branched, or cyclic. Specific examples thereof include alkyl groups having 1 to 20 carbon atoms, such as a methyl group, an ethyl group, an n-propyl group, an isopropyl group, an n-butyl group, an isobutyl group, a sec-butyl group, a tert-butyl group, an n-pentyl group, an n-hexyl group, an n-octyl group, an n-nonyl group, an n-decyl group, an undecyl group, a dodecyl group, a tridecyl group, a tetradecyl group, a pentadecyl group, a heptadecyl group, an octadecyl group, a nonadecyl group, and an icosyl group; cyclic saturated hydrocarbyl groups having 3 to 20 carbon atoms, such as a cyclopropyl group, a cyclopentyl group, a cyclohexyl group, a cyclopropylmethyl group, a 4-methylcyclohexyl group, a cyclohexylmethyl group, a norbornyl group, and an adamantyl group; alkenyl groups having 2 to 20 carbon atoms, such as a vinyl group, an allyl group, a propenyl group, a butenyl group, and a hexenyl group; cyclic unsaturated hydrocarbyl groups having 3 to 20 carbon atoms, such as a cyclohexenyl group; aryl groups having 6 to 20 carbon atoms, such as a phenyl group or a naphthyl group; aralkyl groups having 7 to 20 carbon atoms, such as a benzyl group, a 1-phenylethyl group, and a 2-phenylethyl group; and groups obtained by combining these groups. Some or all of the hydrogen atoms in the hydrocarbyl group are optionally substituted with a group containing a heteroatom such as an oxygen atom, a sulfur atom, a nitrogen atom, or a halogen atom, and some of —CH₂— in the hydrocarbyl group are optionally substituted with a group containing a heteroatom such as an oxygen atom, a sulfur atom, or a nitrogen atom. As a result, the hydrocarbyl group may contain, for instance, a hydroxy group, a cyano group, a fluorine atom, a chlorine atom, a bromine atom, an iodine atom, a carbonyl group, an ether bond, an ester bond, a sulfonate ester bond, a carbonate bond, a lactone ring, a sultone ring, a carboxylic anhydride (—C(═O)—O—C(═O)—), and a haloalkyl group.

In addition, R²² and R²³ are optionally bonded to each other to form a ring together with carbon atoms to which the R²² and R²³ are bonded. Specific examples of the ring formed at this time include a cyclopropane ring, a cyclobutane ring, a cyclopentane ring, a cyclohexane ring, a norbornane ring, and an adamantane ring. Some or all of the hydrogen atoms in the ring are optionally substituted with a group containing a heteroatom such as an oxygen atom, a sulfur atom, a nitrogen atom, or a halogen atom, and some of —CH₂— in the ring are optionally substituted with a group containing a heteroatom such as an oxygen atom, a sulfur atom, or a nitrogen atom. As a result, the ring may contain, for instance, a hydroxy group, a fluorine atom, a chlorine atom, a bromine atom, an iodine atom, a cyano group, a carbonyl group, an ether bond, an ester bond, a sulfonate ester bond, a carbonate bond, a lactone ring, a sultone ring, a carboxylic anhydride (—C(═O)—O—C(═O)—), and a haloalkyl group.

In formula (a3), R²⁴ is a halogen atom, a hydroxy group, a cyano group, a nitro group, a hydrocarbyl group having 1 to 20 carbon atoms and optionally comprising a heteroatom, a hydrocarbyloxy group having 1 to 20 carbon atoms and optionally comprising a heteroatom, a hydrocarbyloxycarbonyl group having 2 to 20 carbon atoms and optionally comprising a heteroatom, a hydrocarbylthio group having 1 to 20 carbon atoms and optionally comprising a heteroatom, or —N(R^{24A})(R^{24B}) where R^{24A} and R^{24B} are each independently a hydrogen atom or a hydrocarbyl group having 1 to 6 carbon atoms. The halogen atom is preferably a fluorine atom, a chlorine atom, a bromine atom, and an iodine atom, and more preferably a fluorine atom or an iodine atom. The hydrocarbyl group and hydrocarbyl moieties of the hydrocarbyloxy group, hydrocarbyloxycarbonyl group, and hydrocarbylthio group may be either saturated or unsaturated and linear, branched, or cyclic. Specific examples thereof include the same hydrocarbyl group represented by R²² and R²³ as illustrated. Some or all of the hydrogen atoms in the hydrocarbyl group are optionally substituted with a group containing a heteroatom such as an oxygen atom, a sulfur atom, a nitrogen atom, or a halogen atom, and some of —CH₂— in the hydrocarbyl group are optionally substituted with a group containing a heteroatom such as an oxygen atom, a sulfur atom, or a nitrogen atom. As a result, the hydrocarbyl group may contain, for instance, a hydroxy group, a cyano group, a fluorine atom, a chlorine atom, a bromine atom, an iodine atom, a carbonyl group, an ether bond, an ester bond, a sulfonate ester bond, a carbonate bond, a lactone ring, a sultone ring, a carboxylic anhydride (—C(═O)—O—C(═O)—), and a haloalkyl group. When “b2” is 2 or more, R²⁴ are the same as or different from each other.

In addition, when “b2” is 2 or more, a plurality of R²⁴ are optionally bonded to each other to form a ring together with carbon atoms of an aromatic ring to which the R²⁴ are bonded. Specific examples of the ring formed at this time include a cyclopropane ring, a cyclobutane ring, a cyclopentane ring, a cyclohexane ring, a norbornane ring, and an adamantane ring. Some or all of the hydrogen atoms in the ring are optionally substituted with a group containing a heteroatom such as an oxygen atom, a sulfur atom, a nitrogen atom, or a halogen atom, and some of —CH₂— in the ring are optionally substituted with a group containing a heteroatom such as an oxygen atom, a sulfur atom, or a nitrogen atom. As a result, the ring may contain, for instance, a hydroxy group, a fluorine atom, a chlorine atom, a bromine atom, an iodine atom, a cyano group, a carbonyl group, an ether bond, an ester bond, a sulfonate ester bond, a carbonate bond, a lactone ring, a sultone ring, a carboxylic anhydride (—C(═O)—O—C(═O)—), and a haloalkyl group.

Specific examples of the repeating unit a3 are given below, but not limited thereto. In formulae below, R^{A} is the same as above, and Me represents a methyl group. The bonding positions of substituents on the aromatic ring are interchangeable.

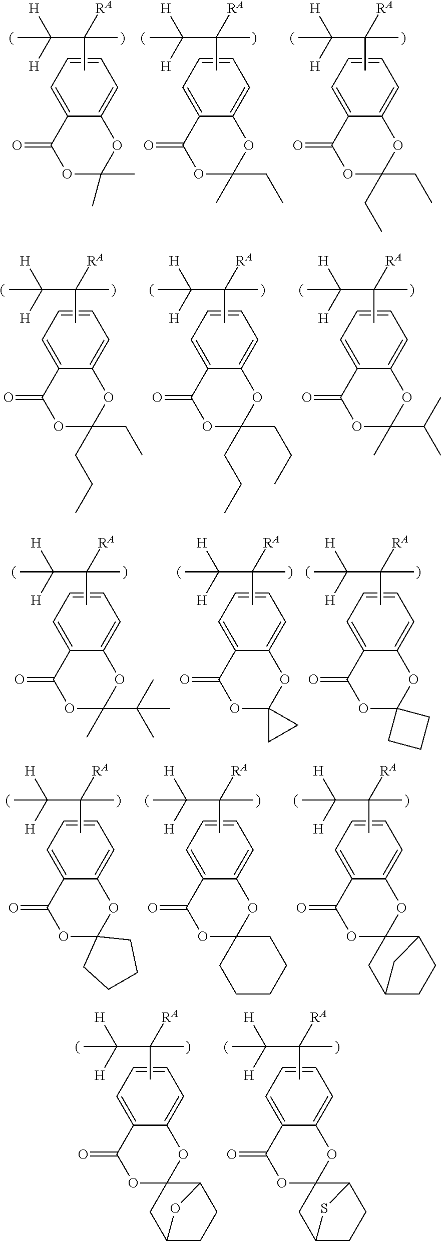

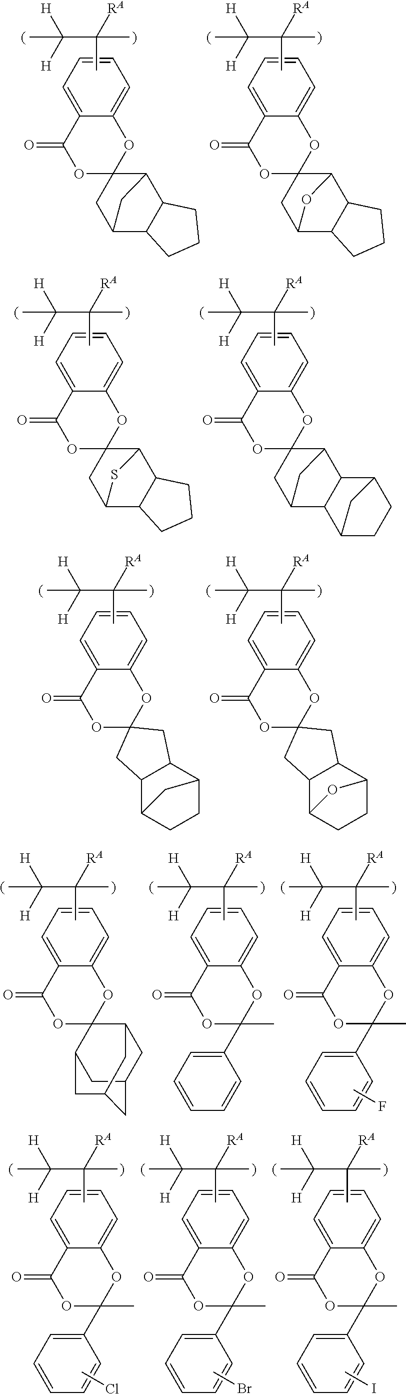

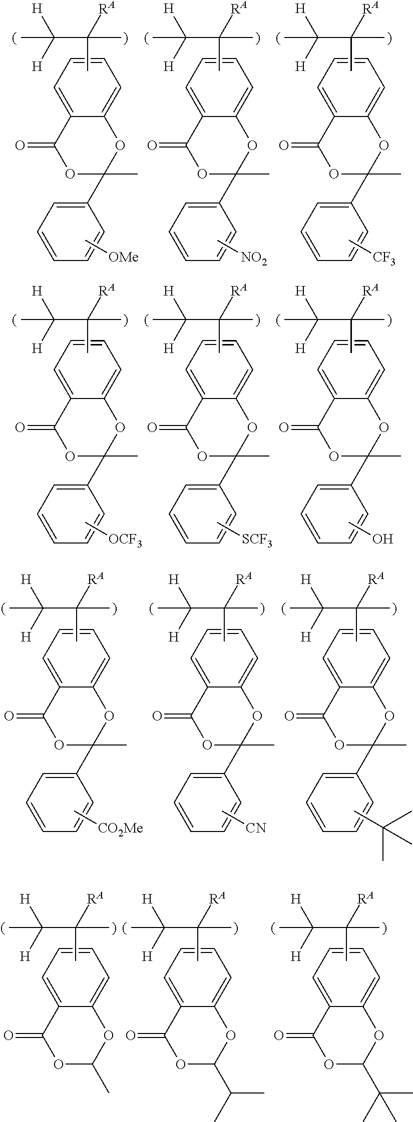

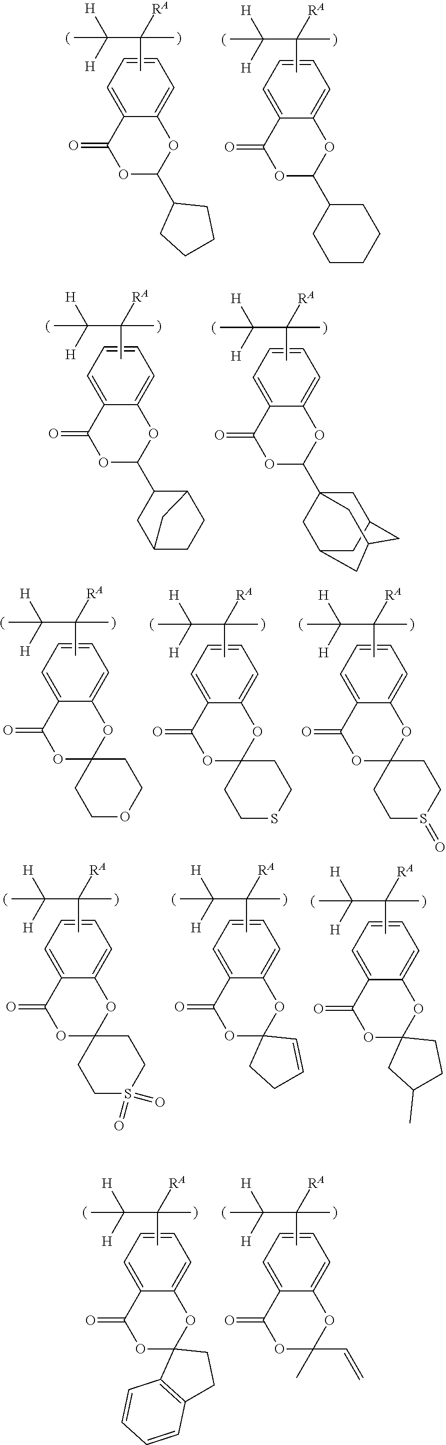

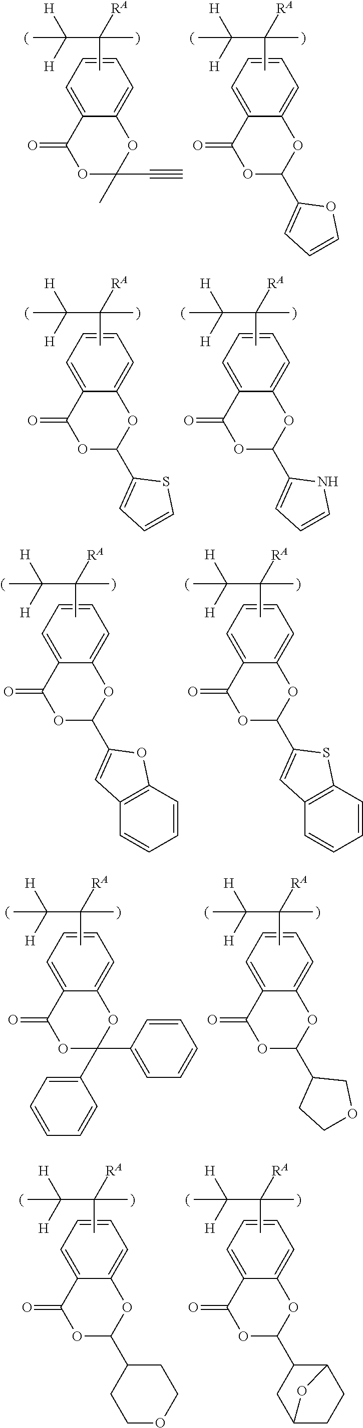

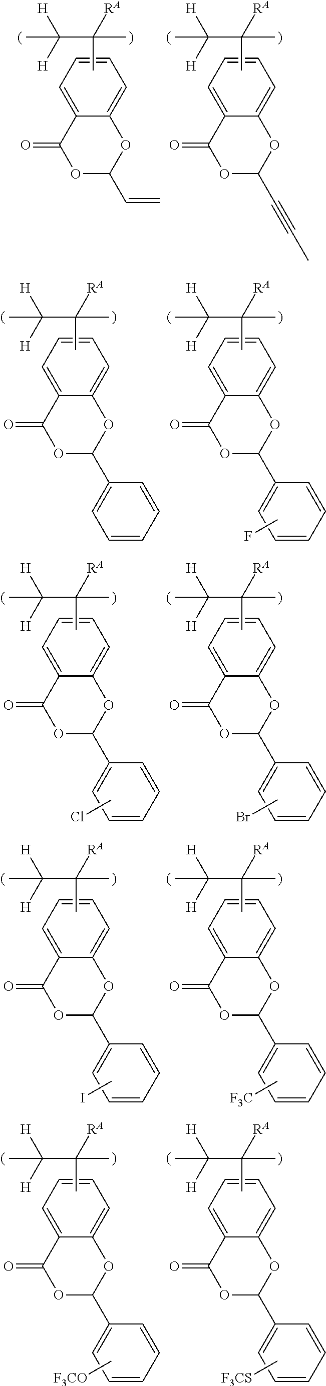

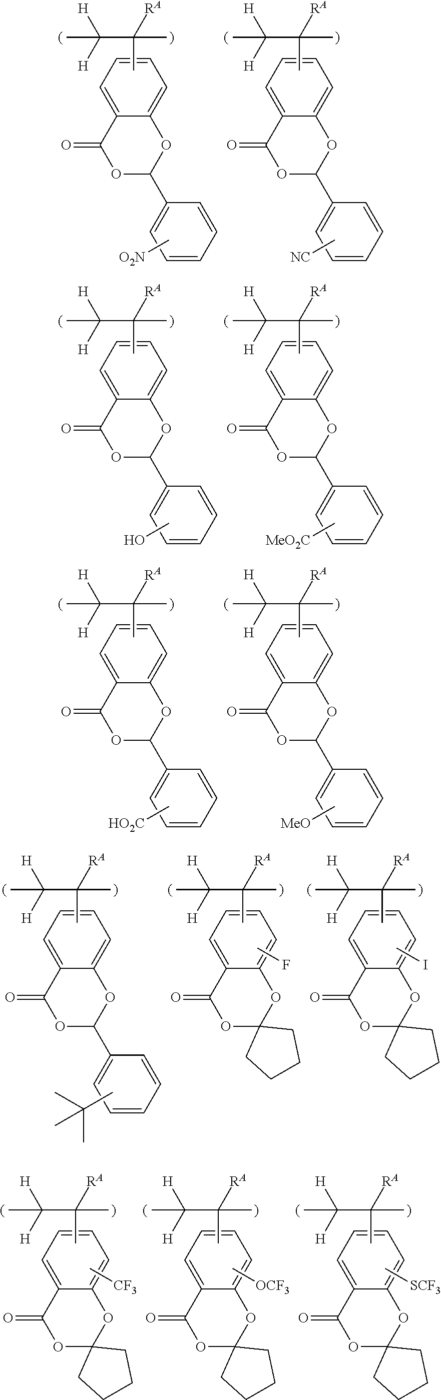

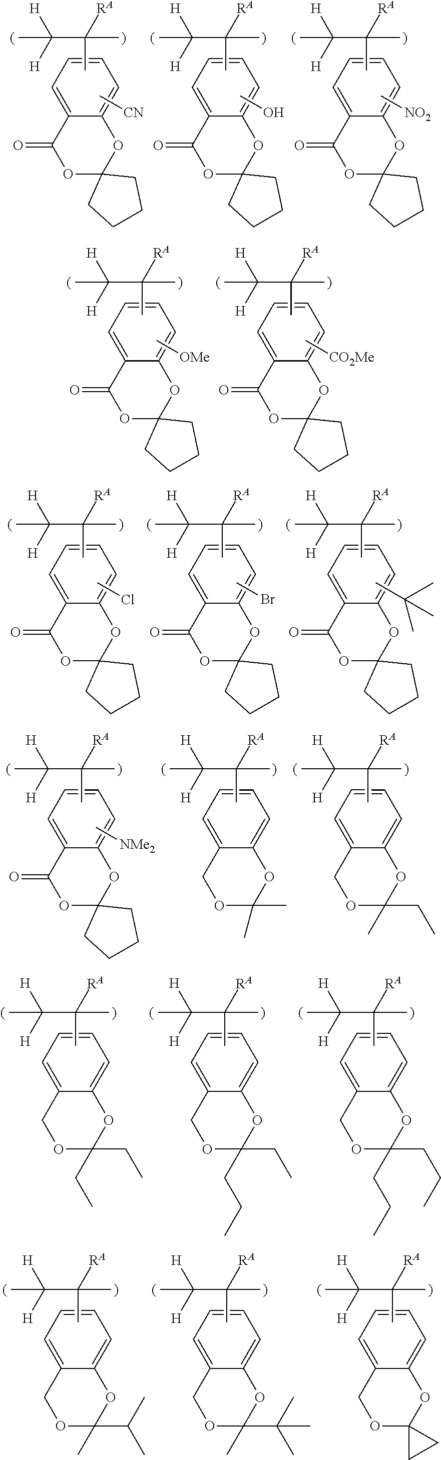

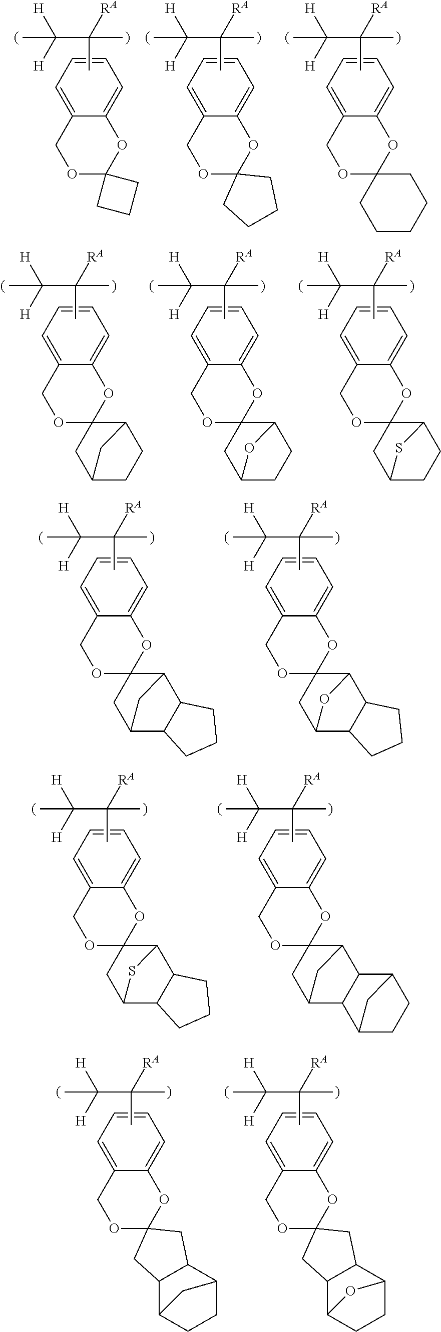

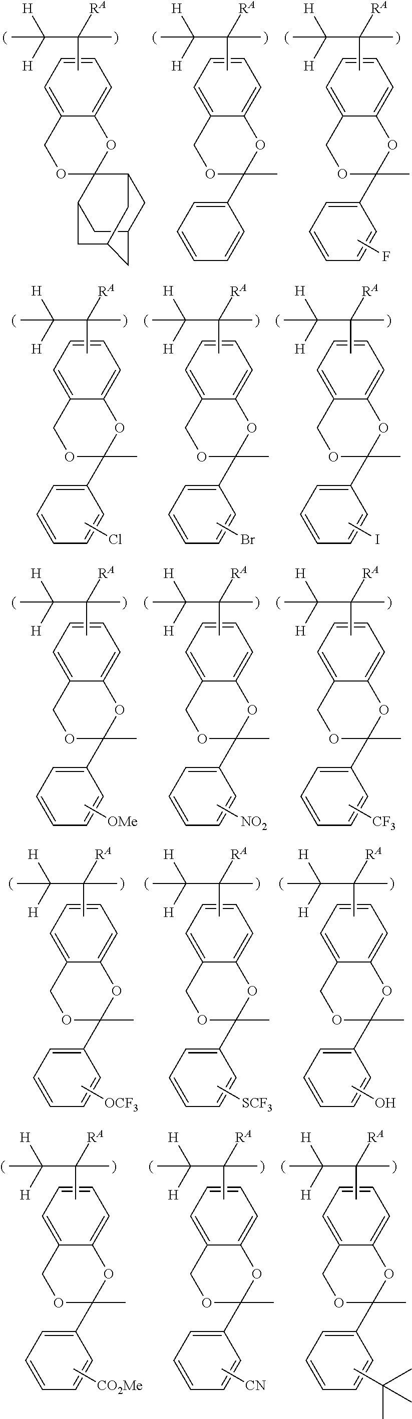

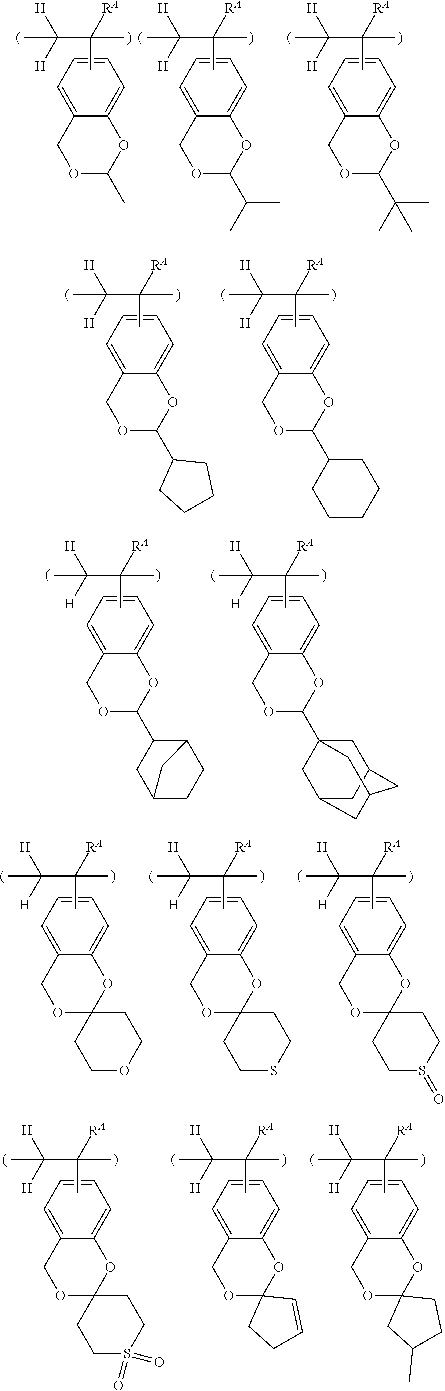

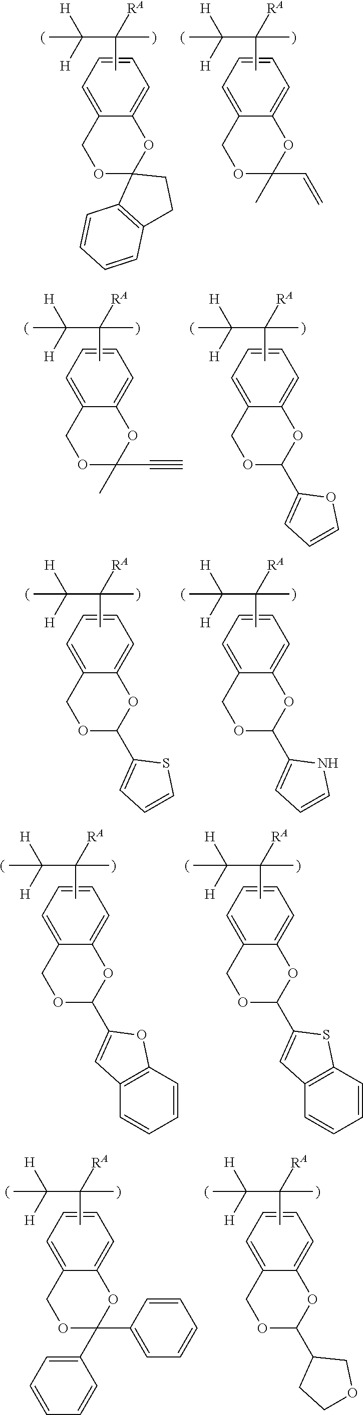

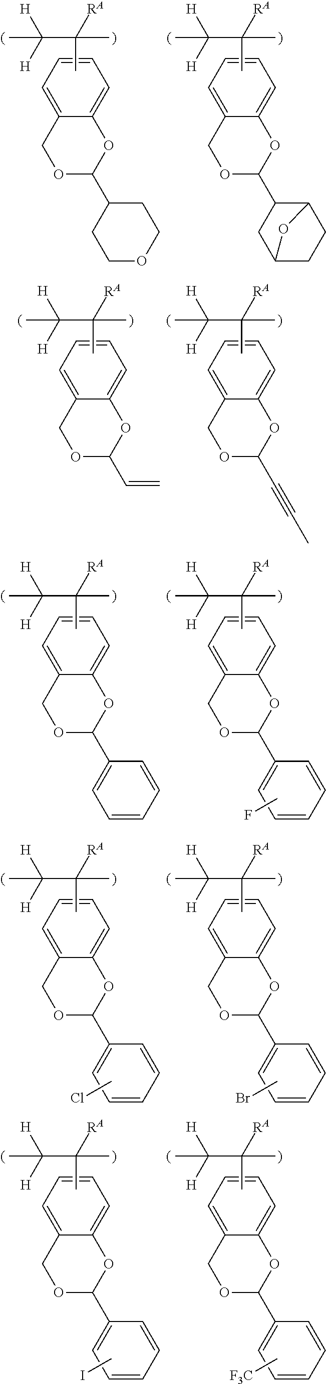

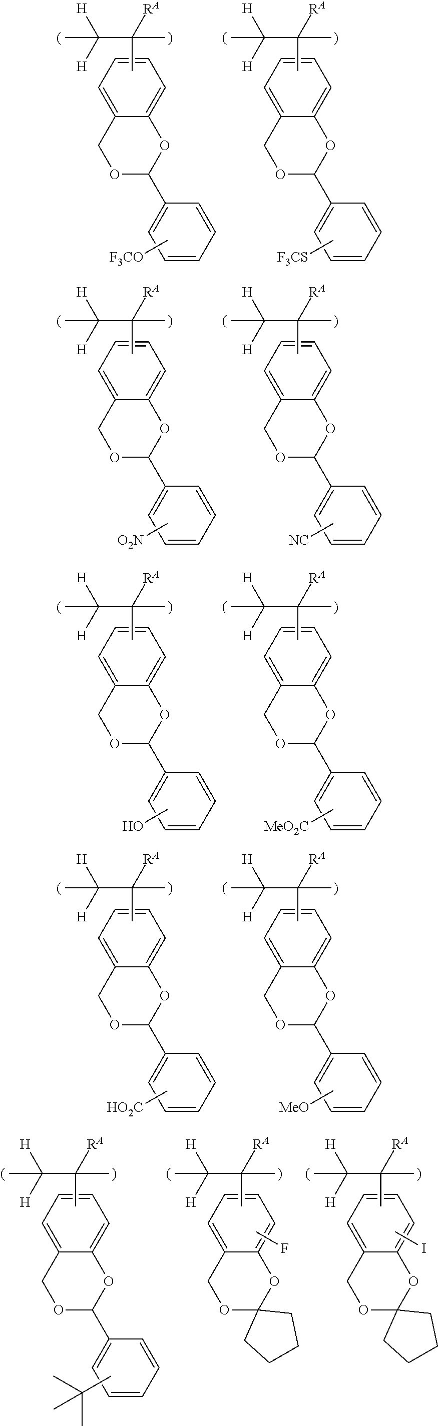

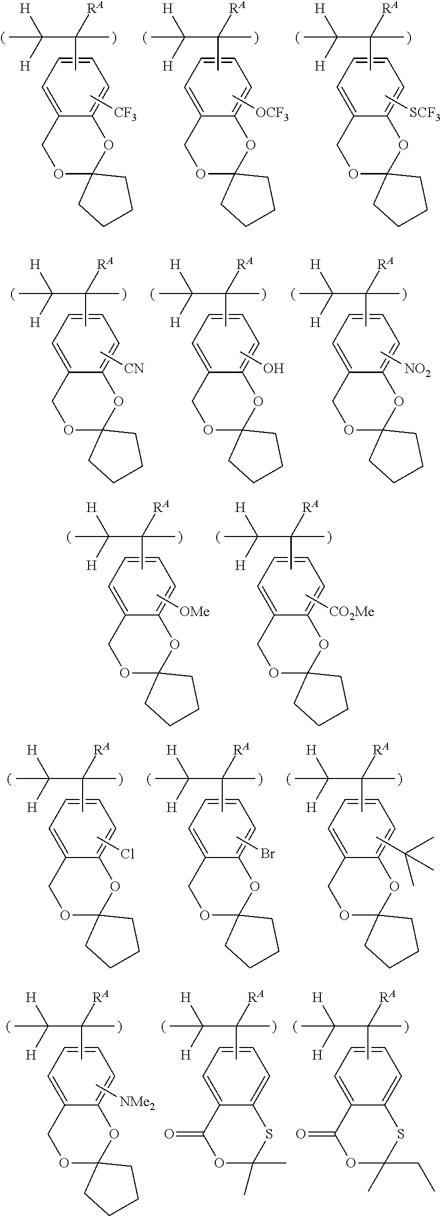

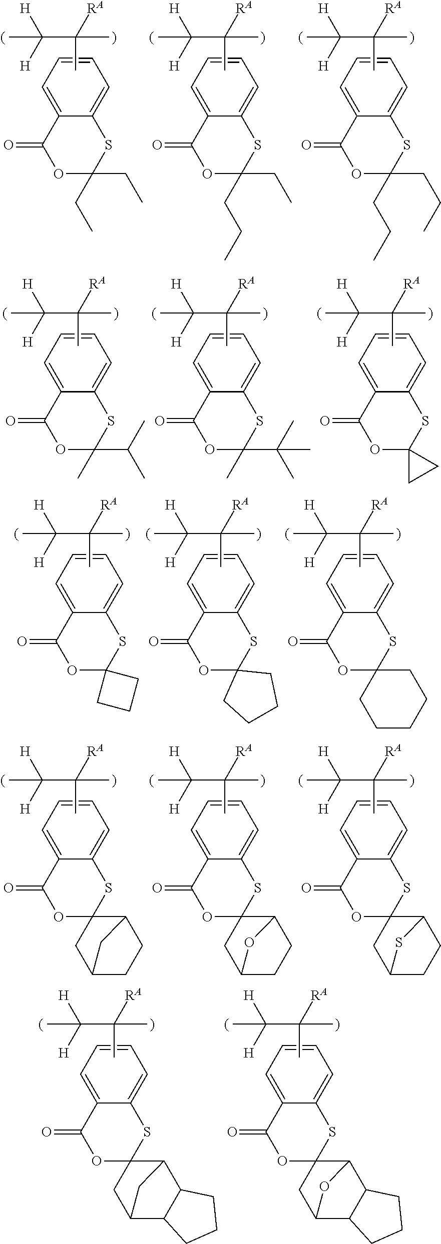

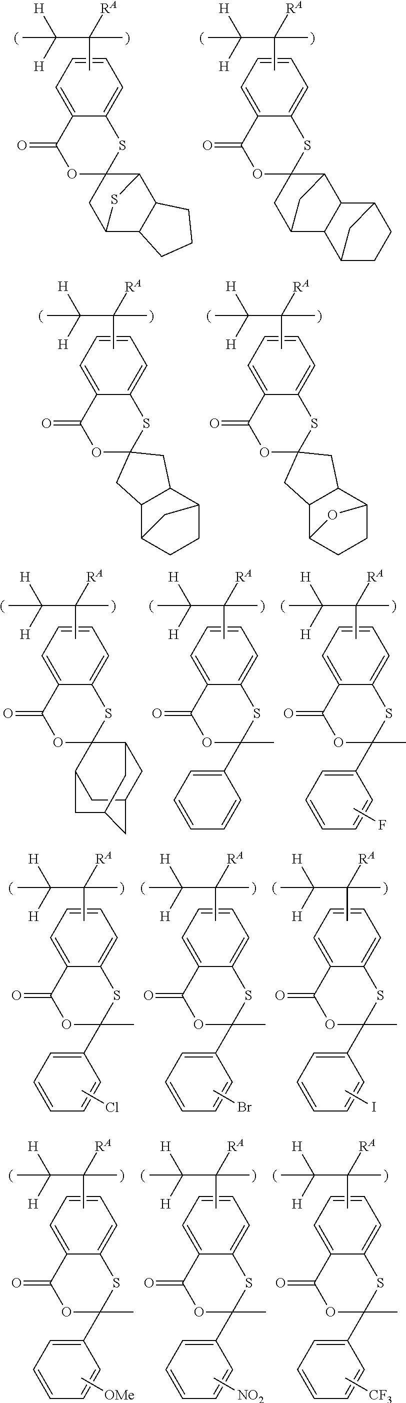

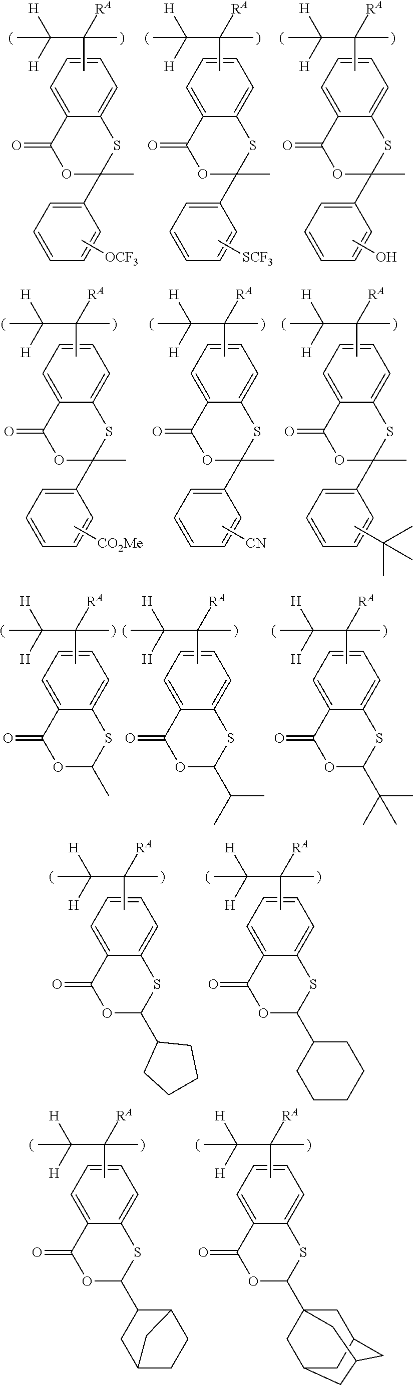

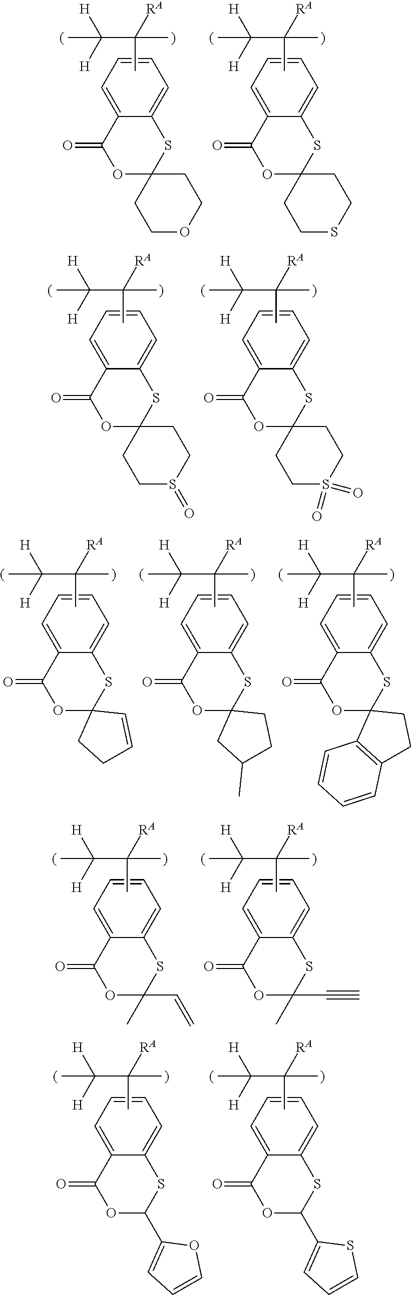

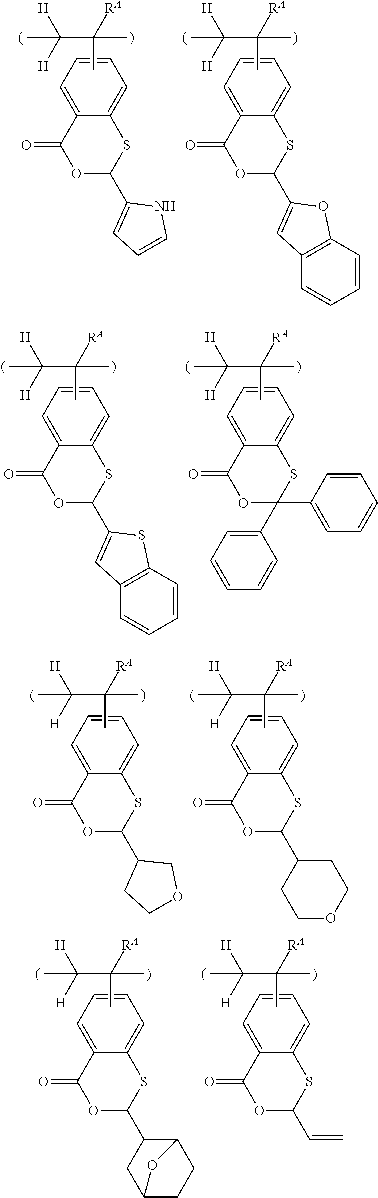

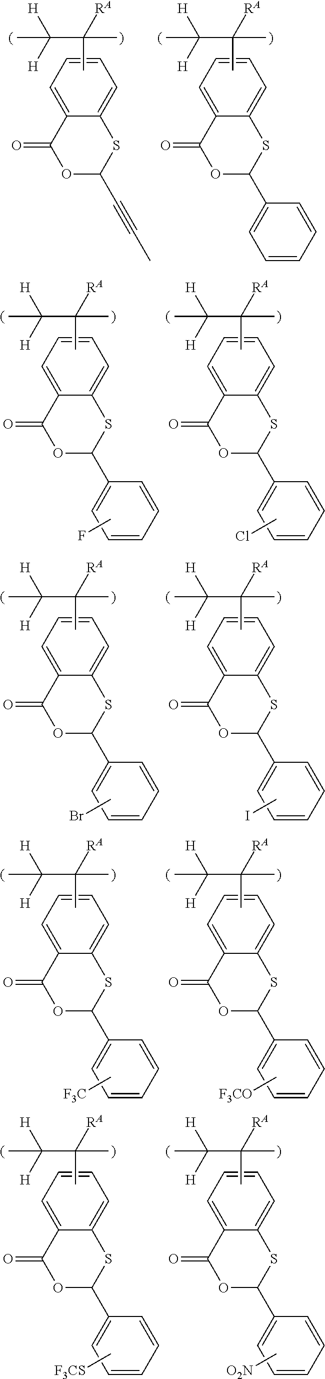

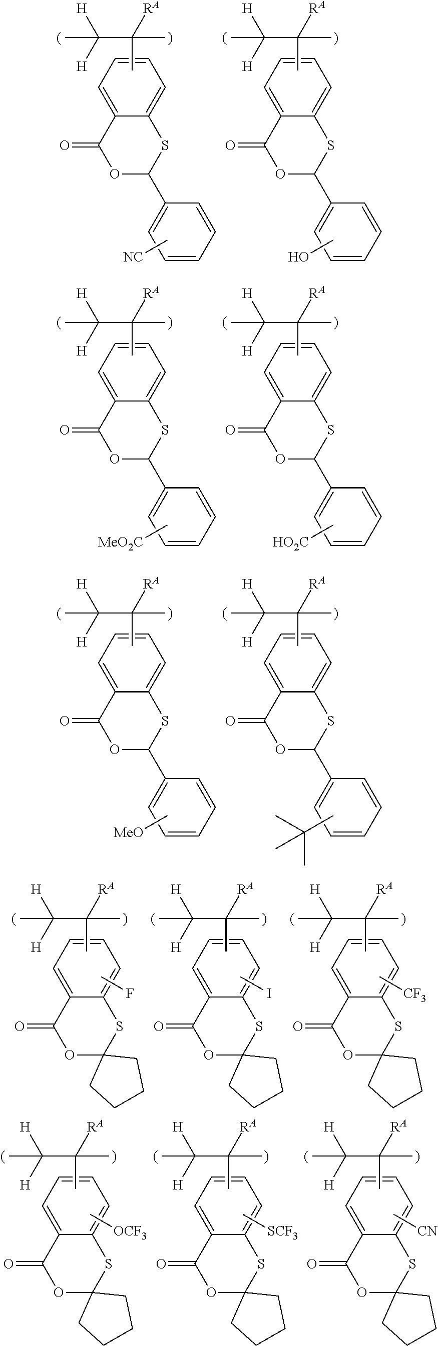

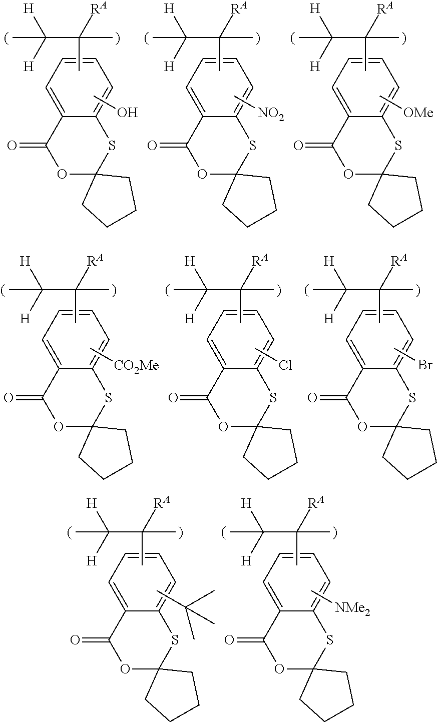

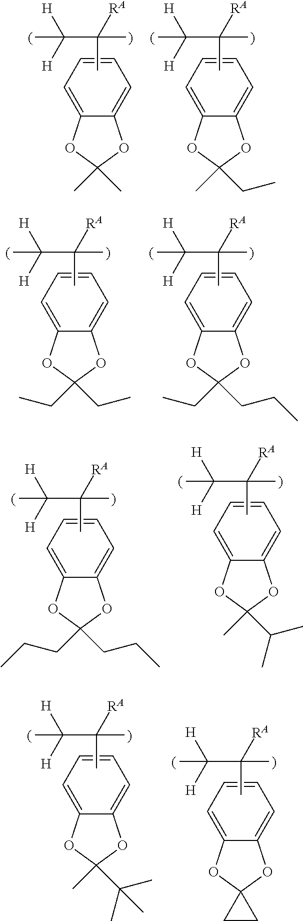

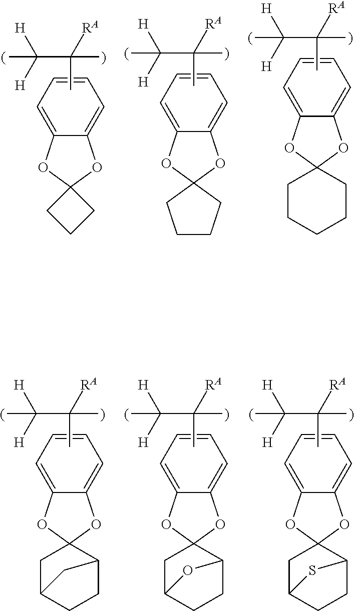

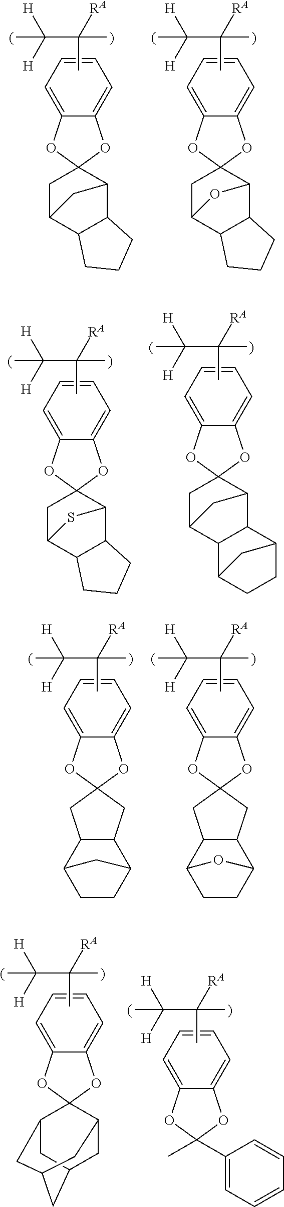

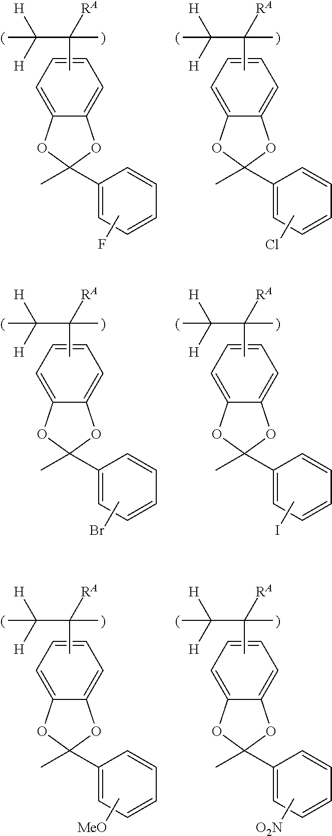

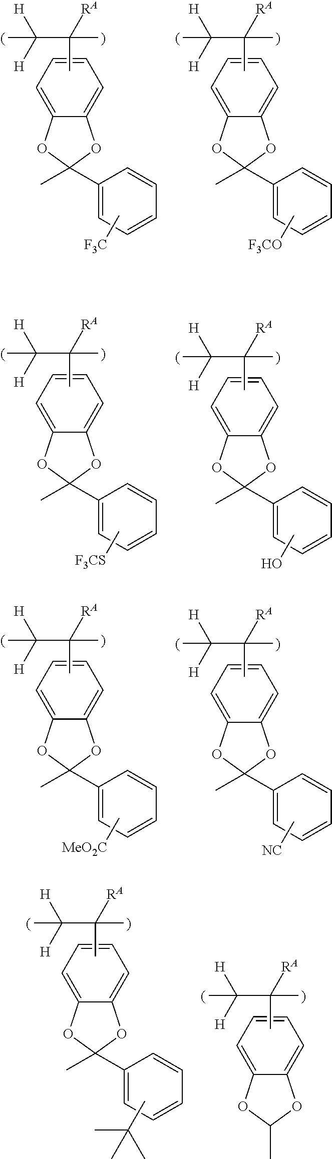

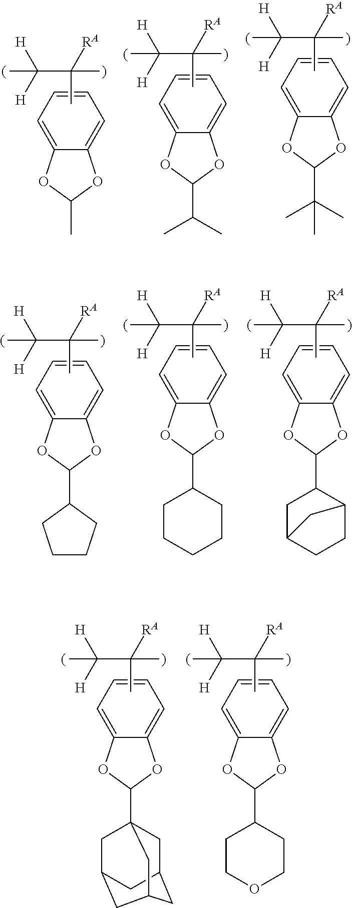

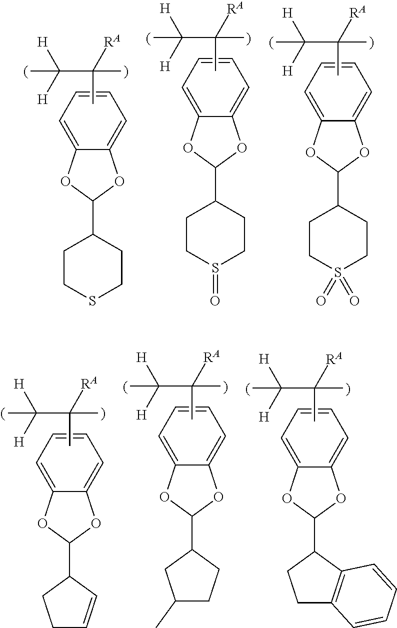

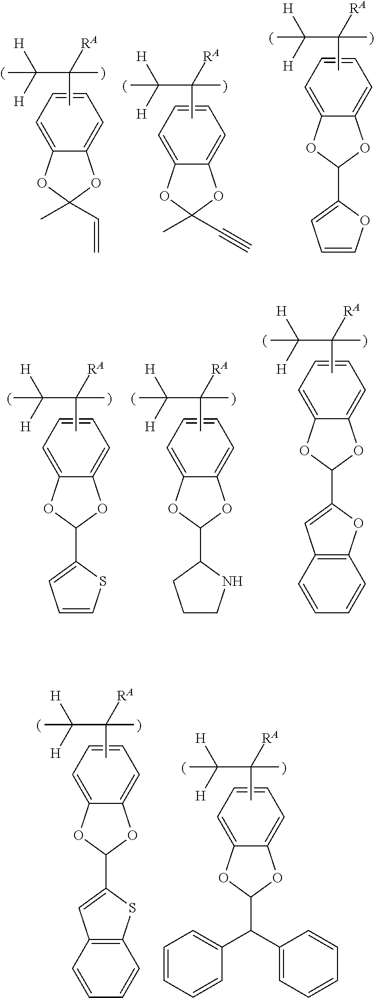

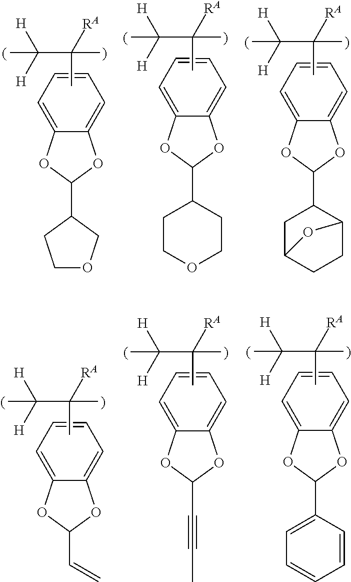

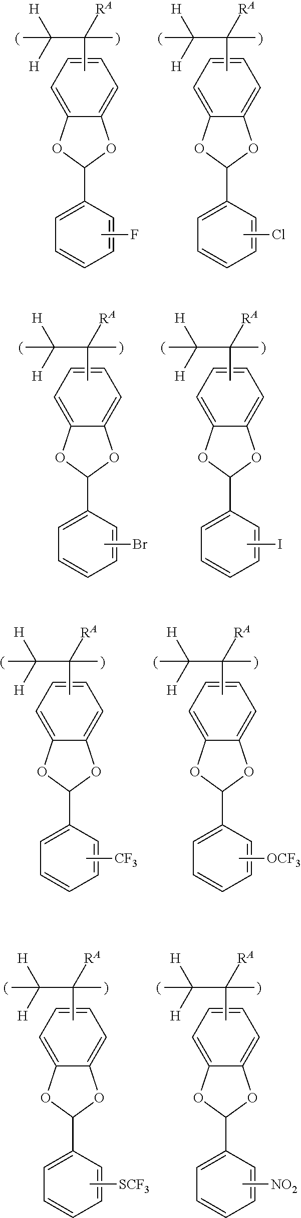

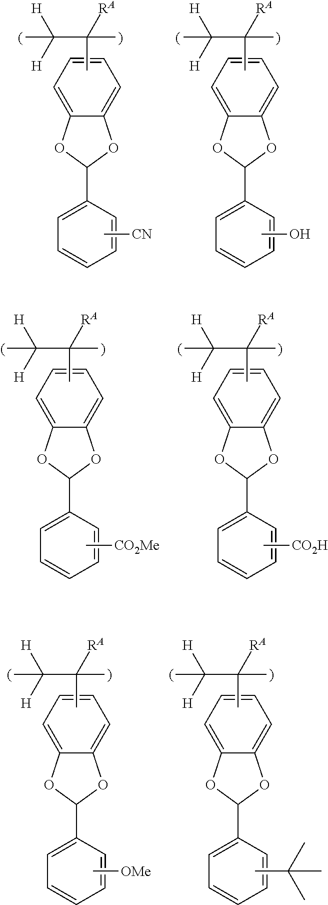

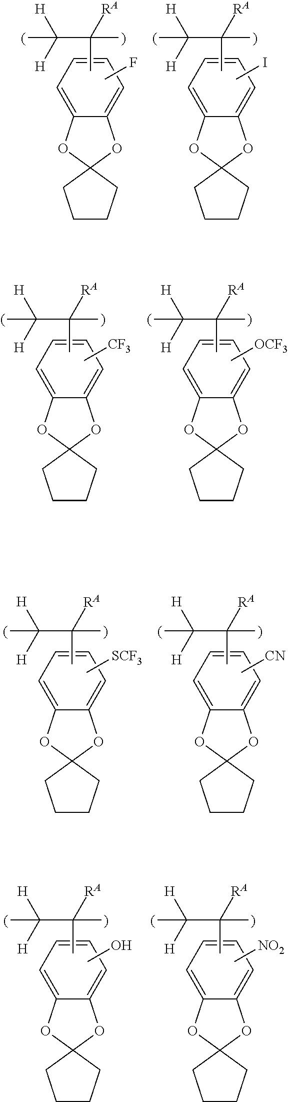

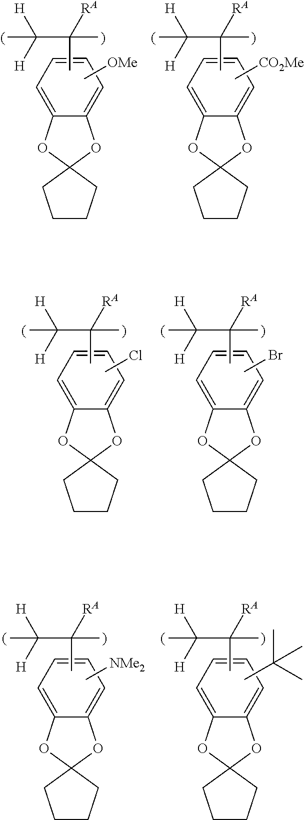

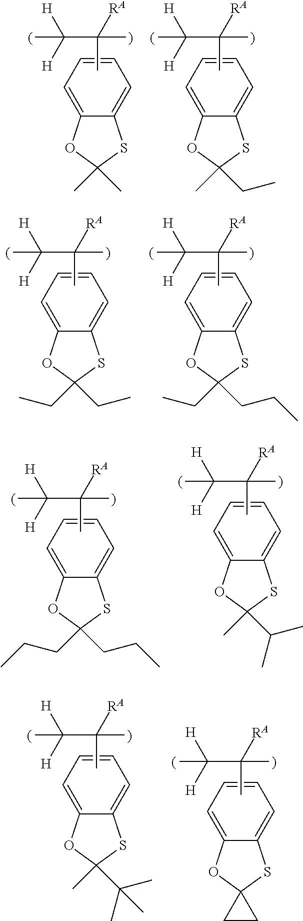

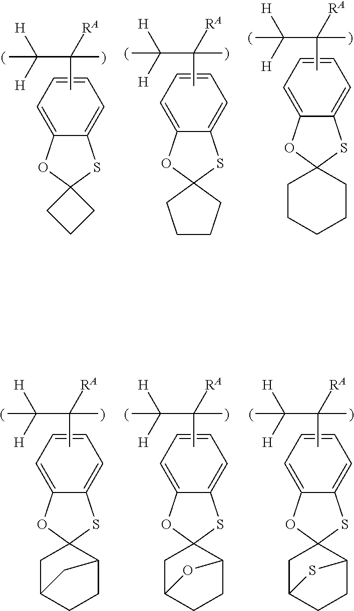

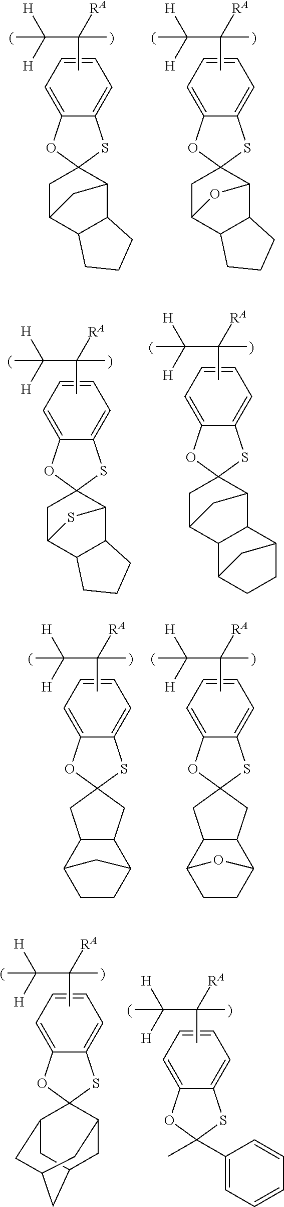

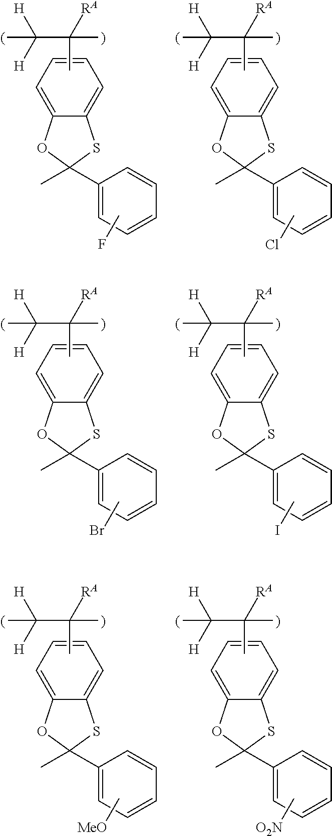

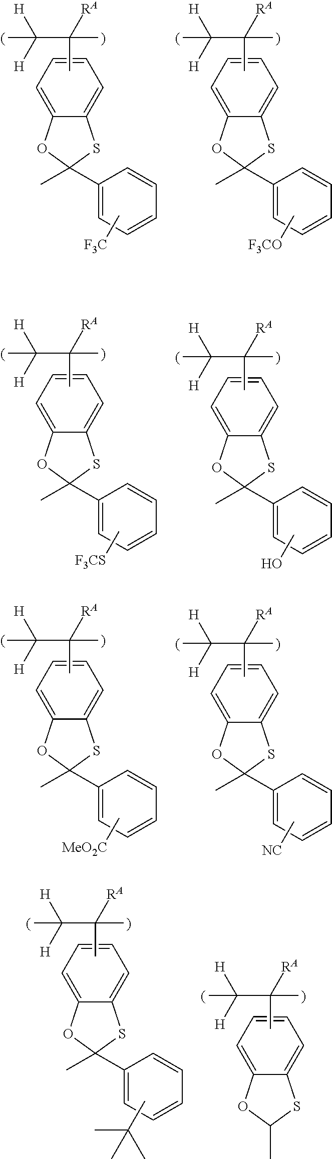

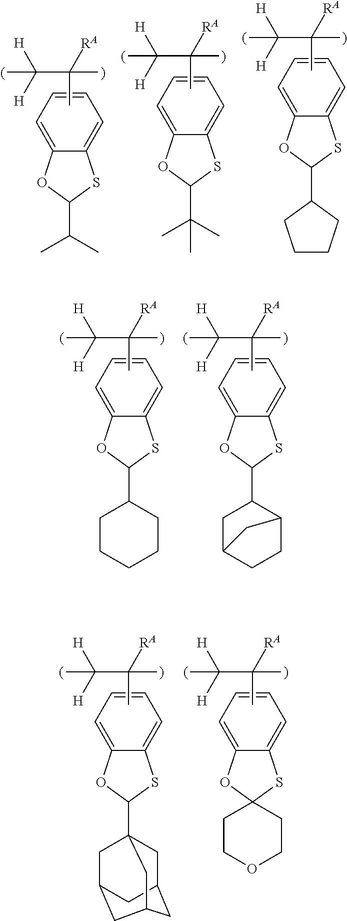

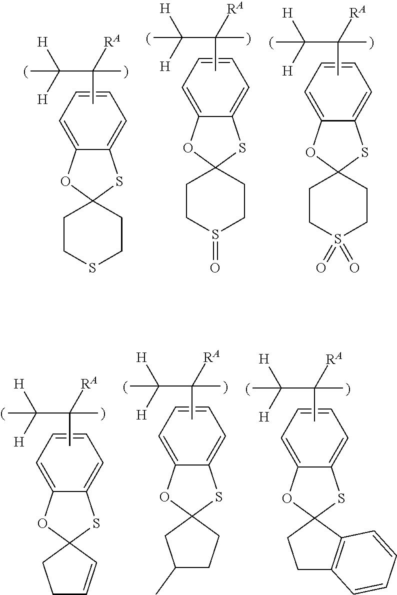

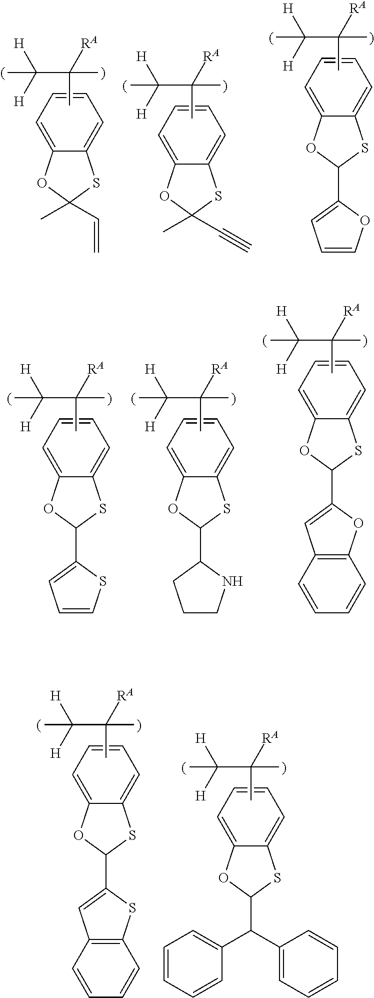

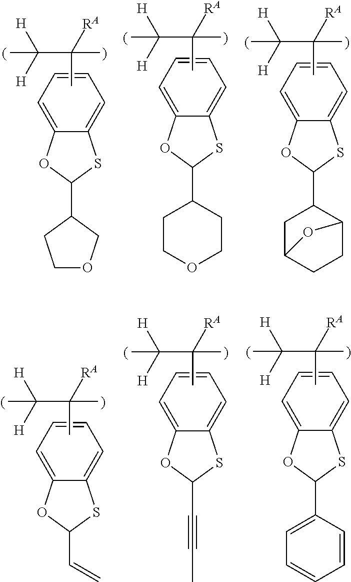

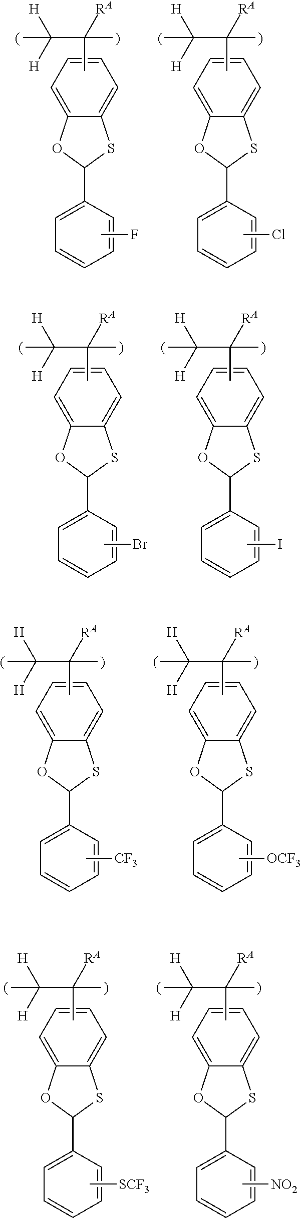

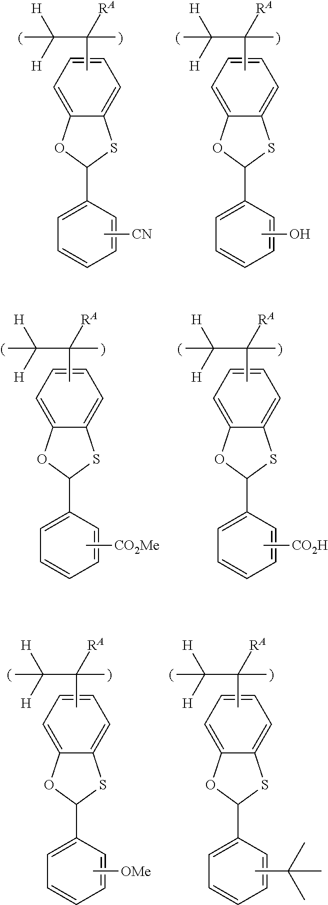

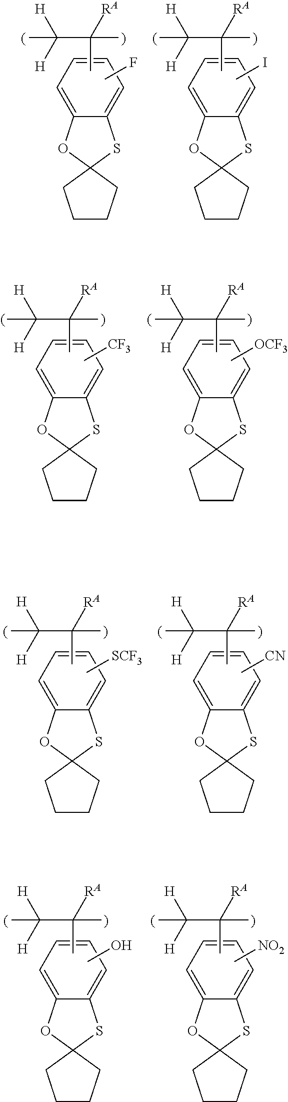

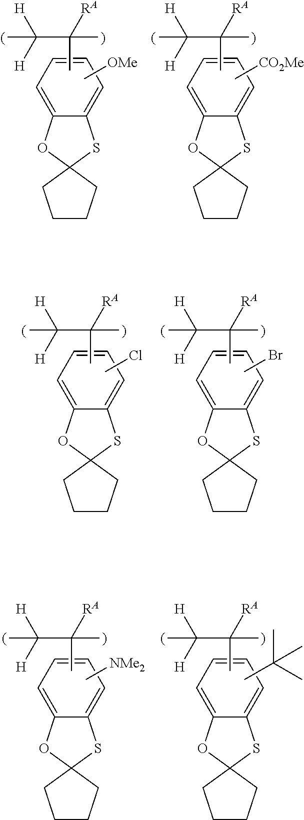

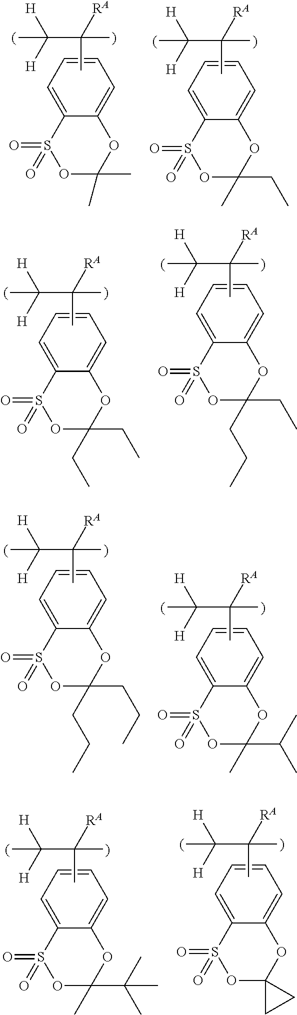

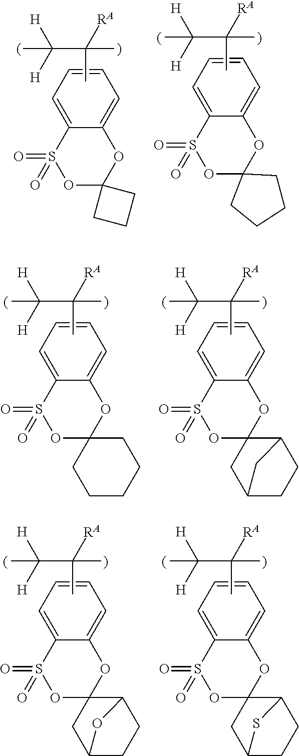

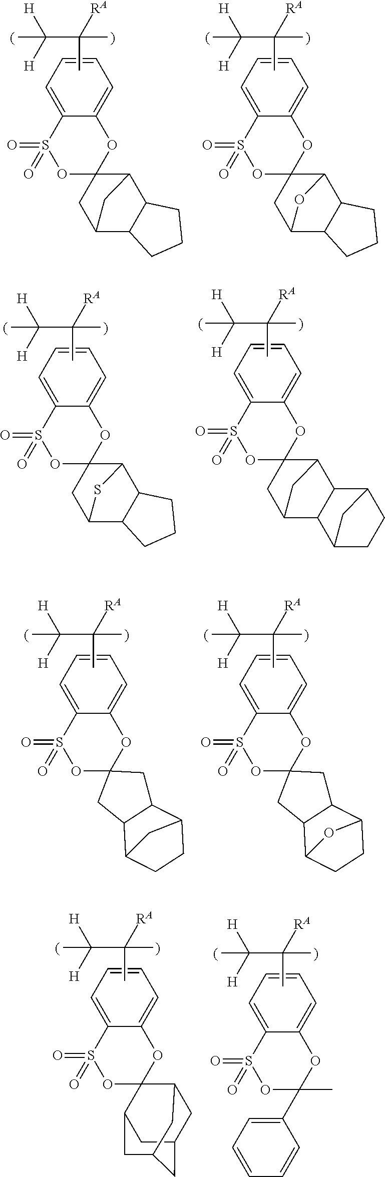

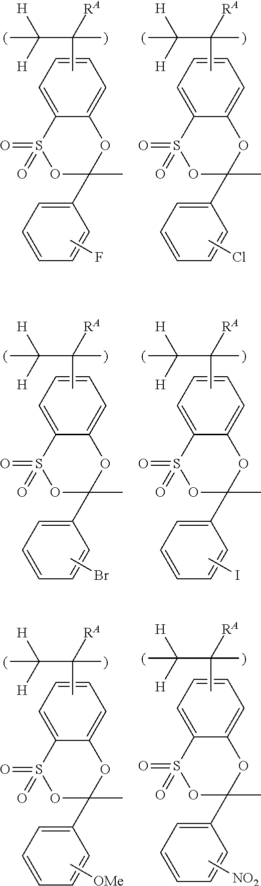

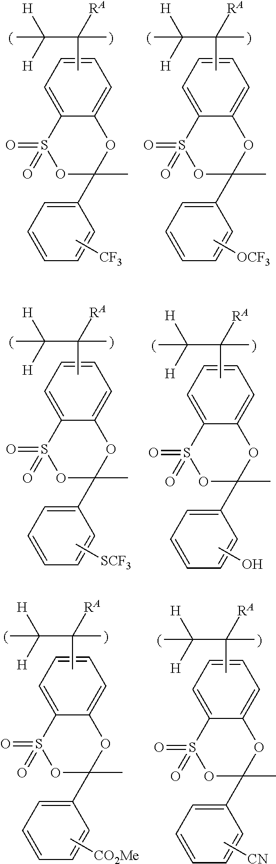

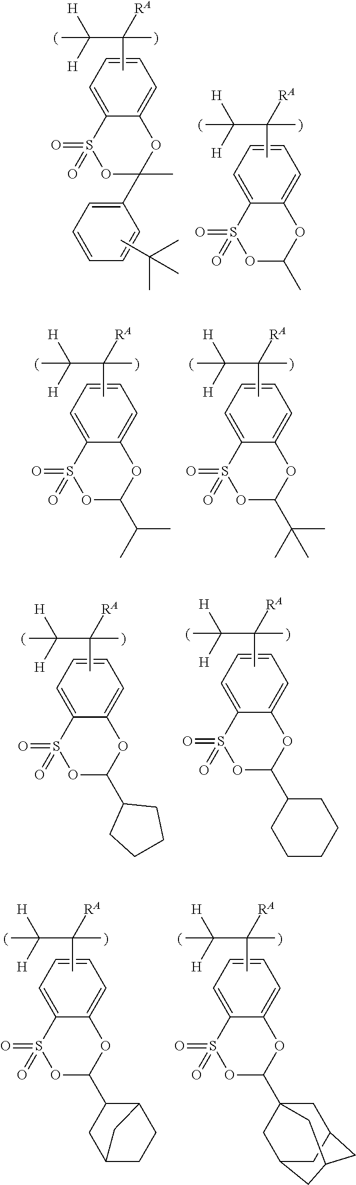

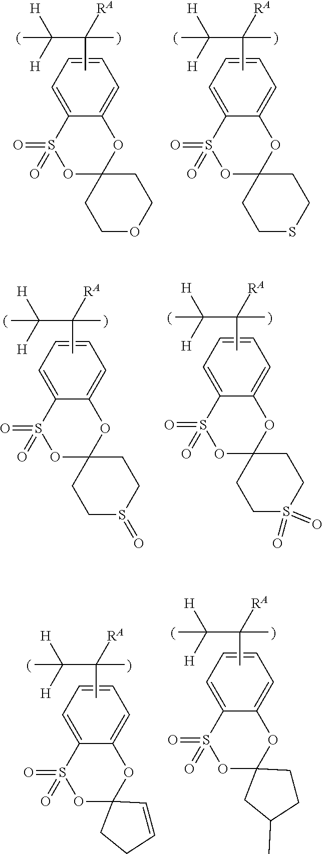

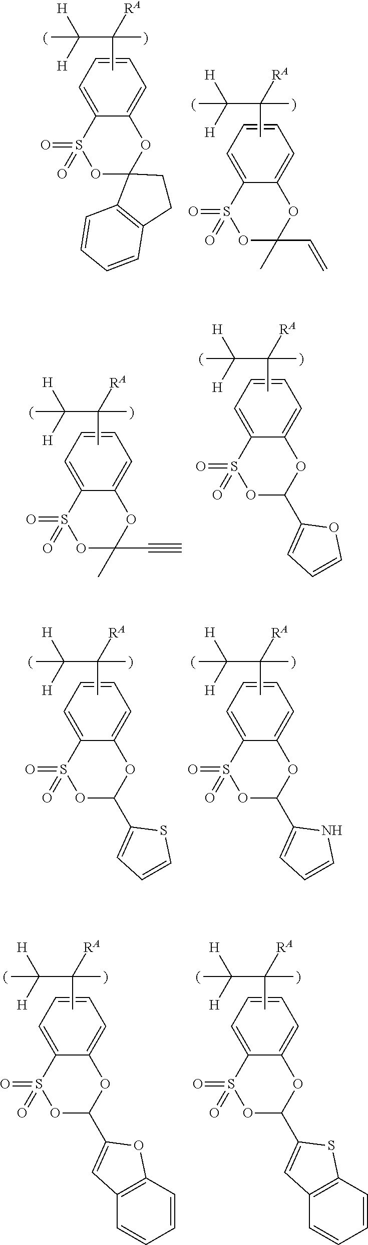

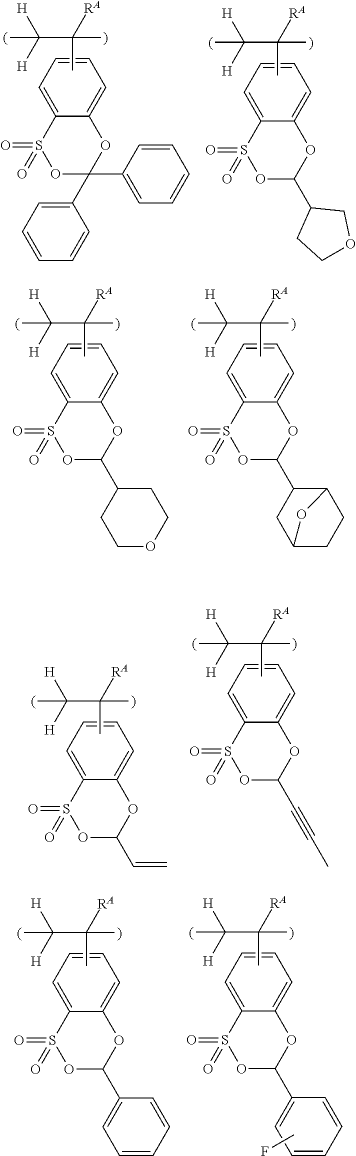

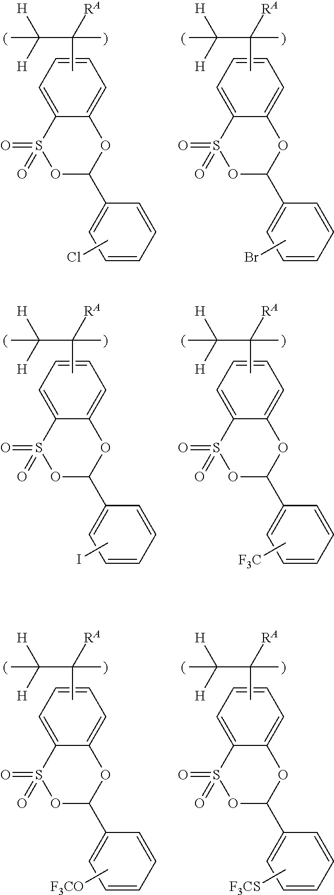

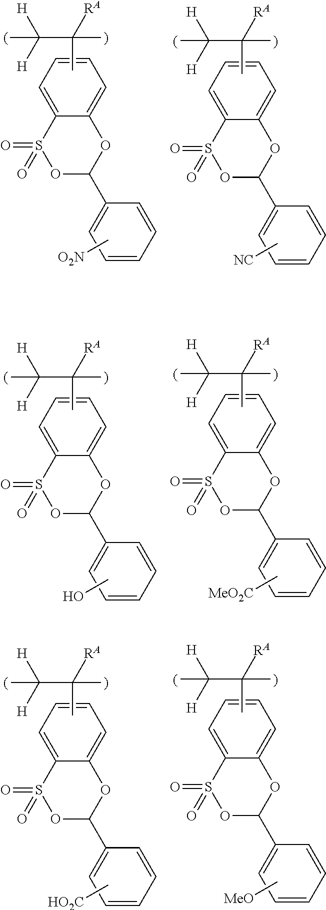

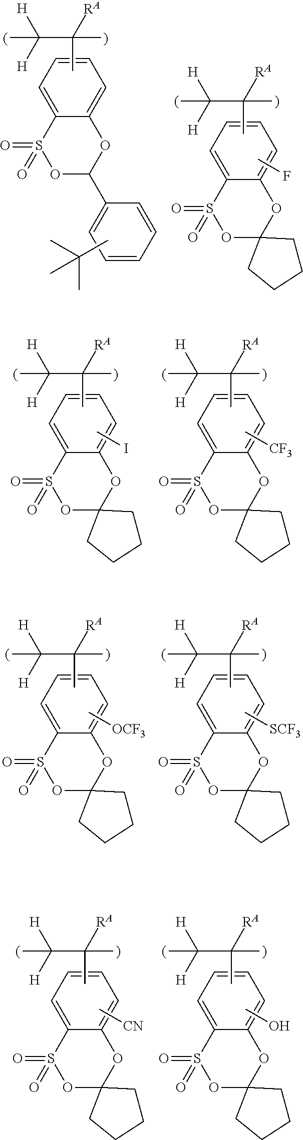

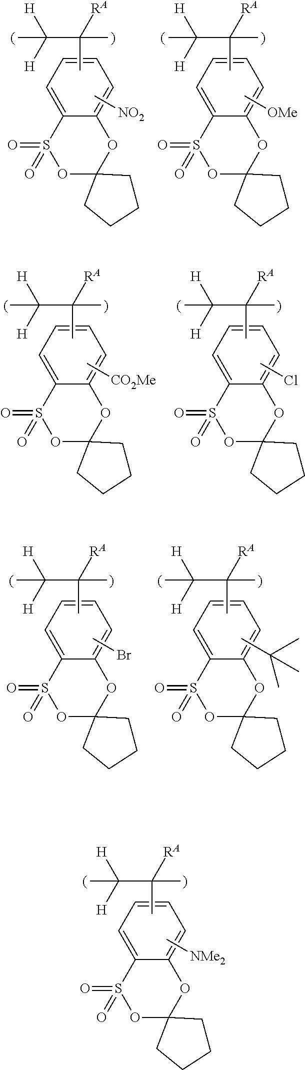

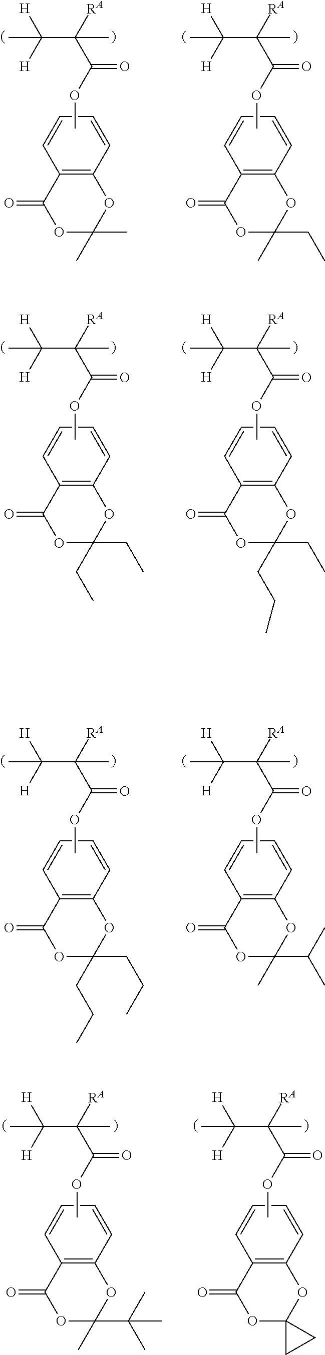

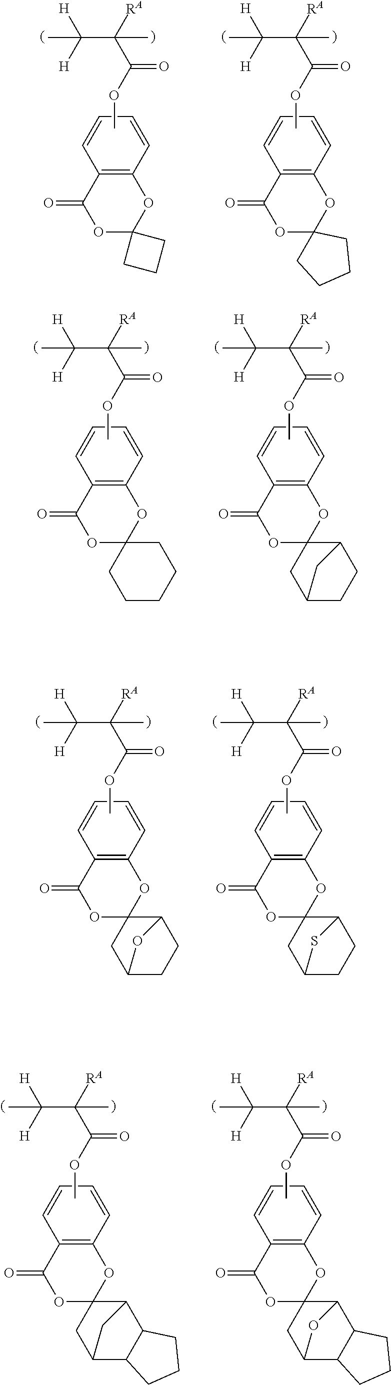

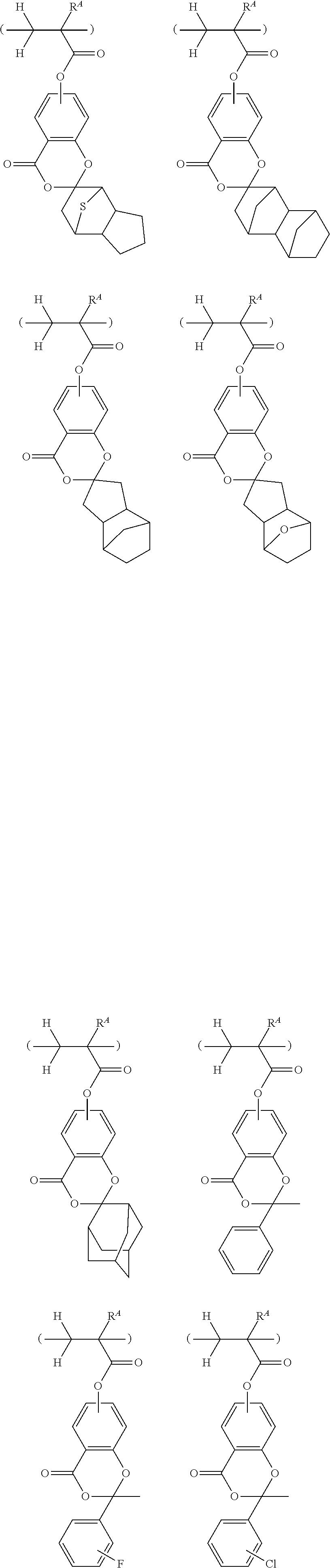

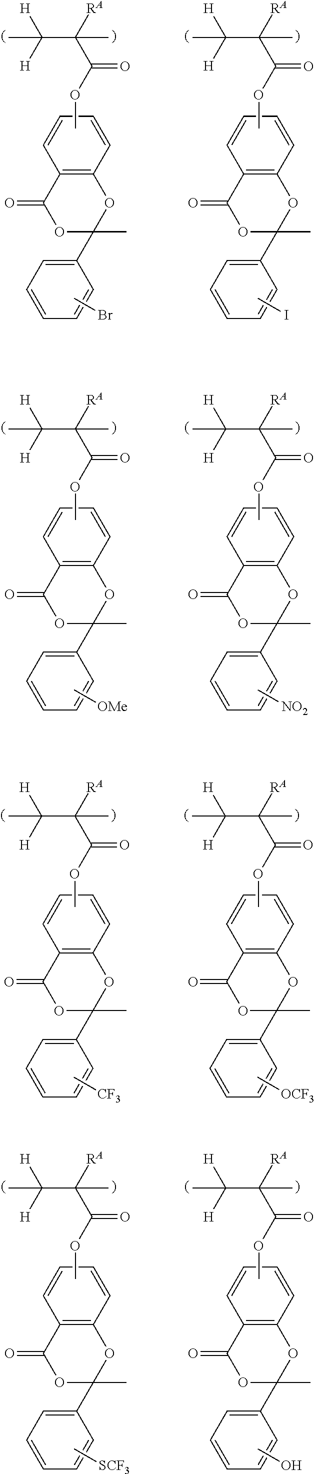

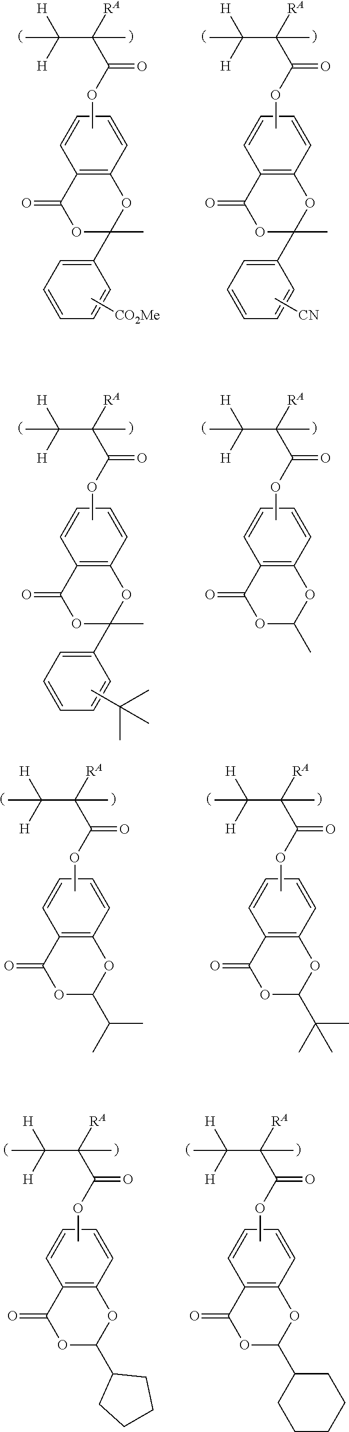

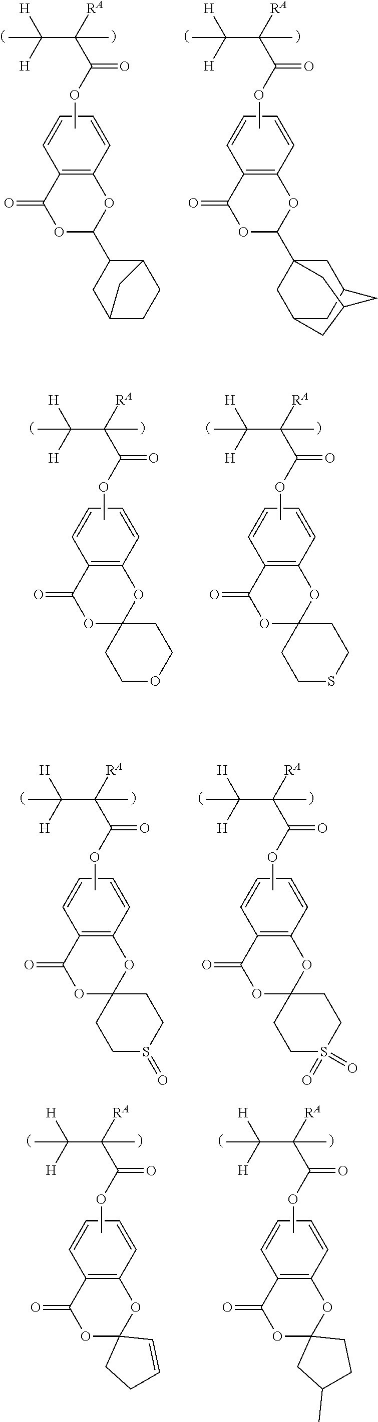

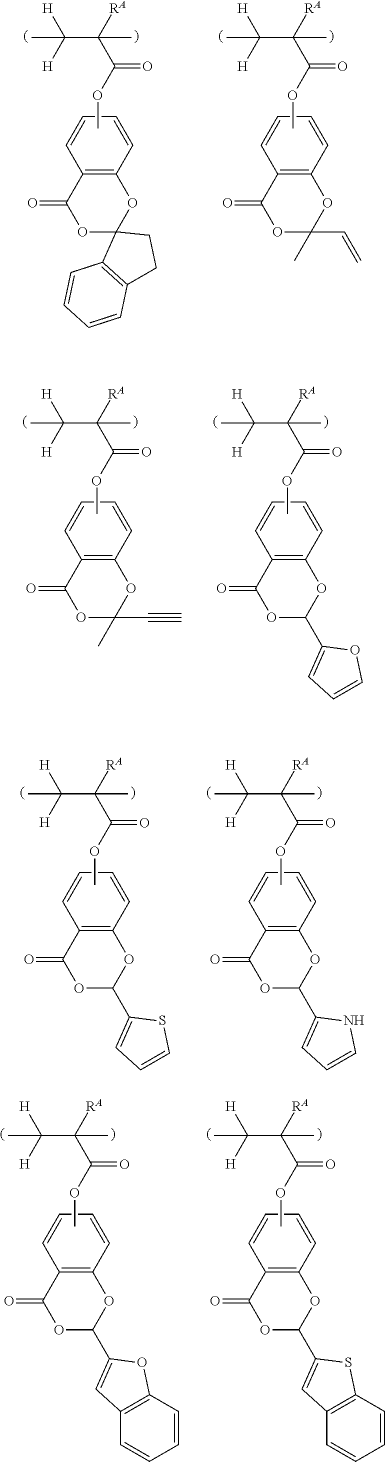

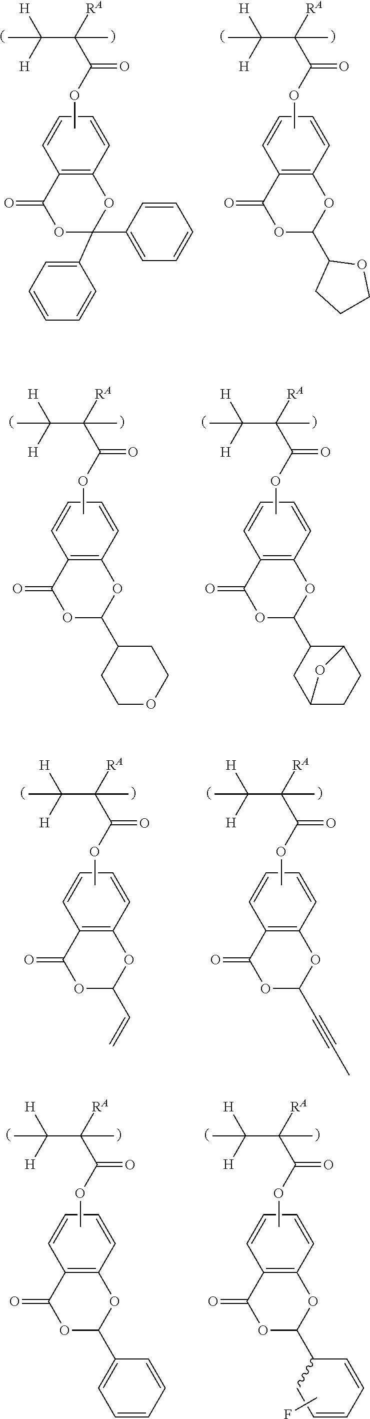

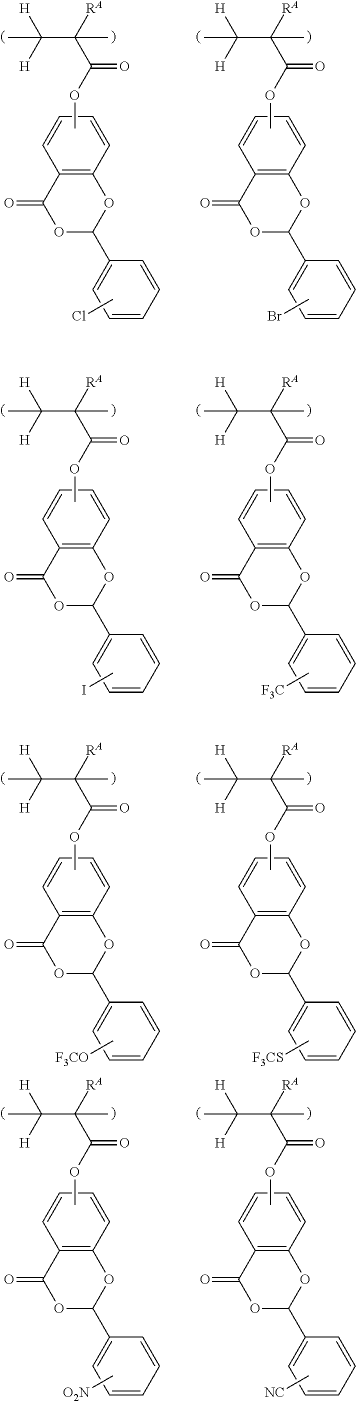

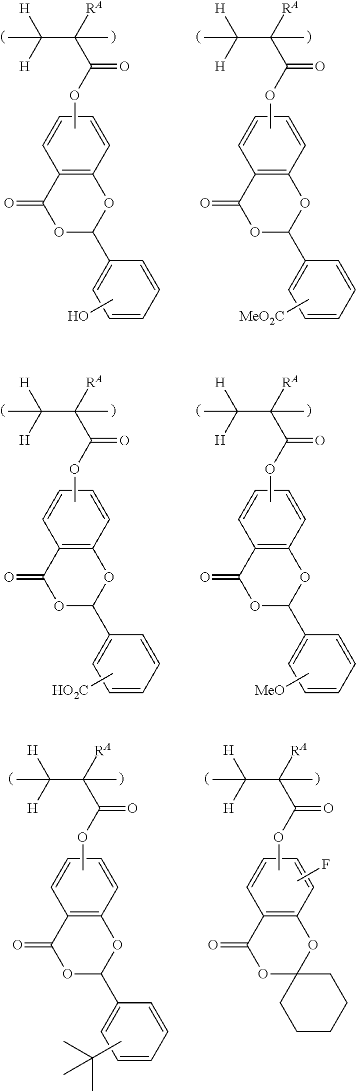

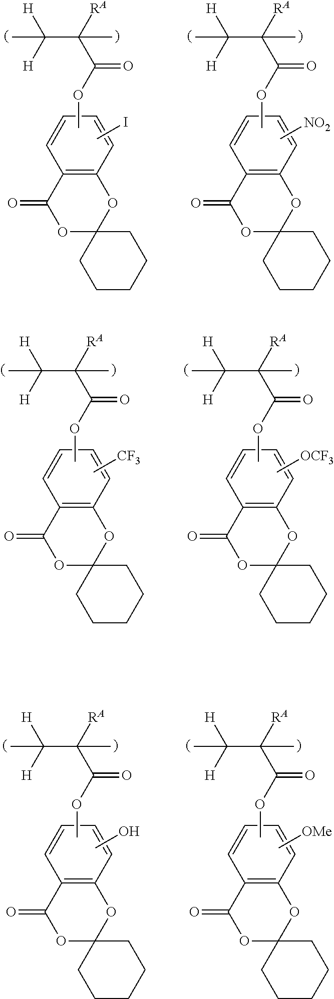

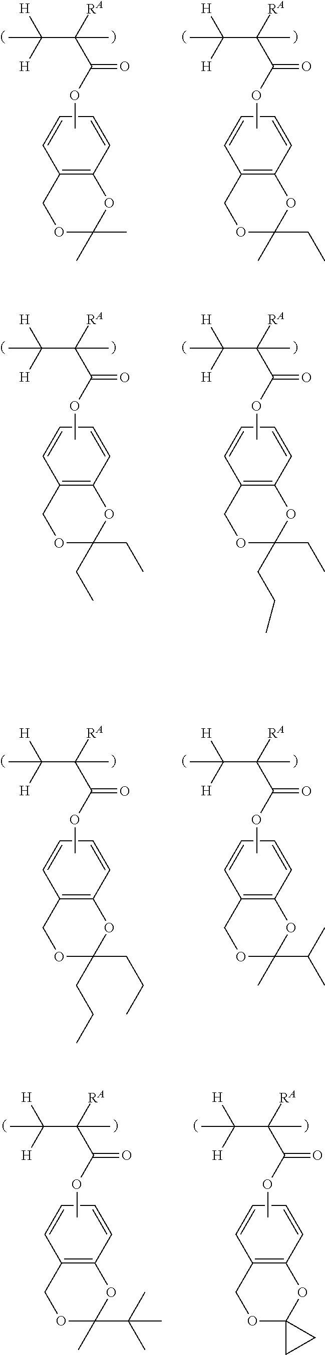

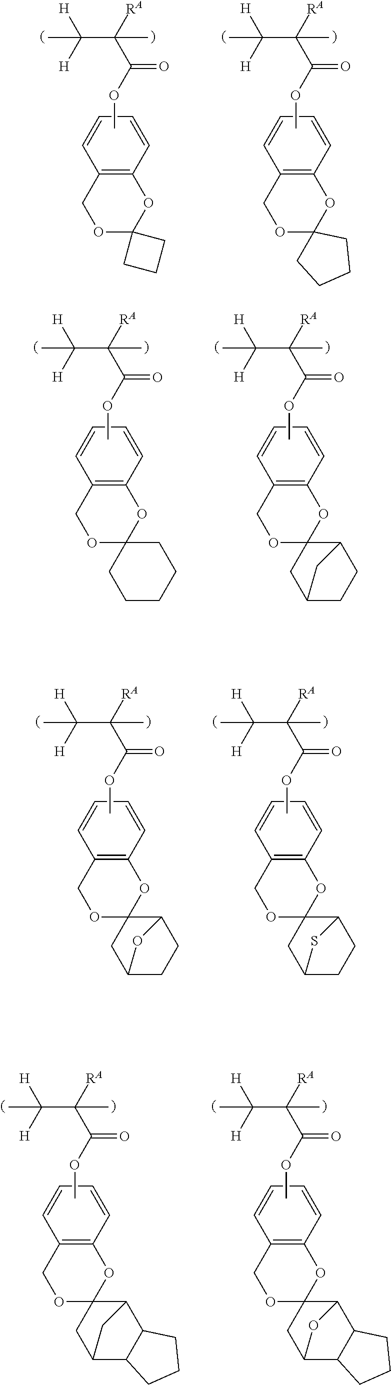

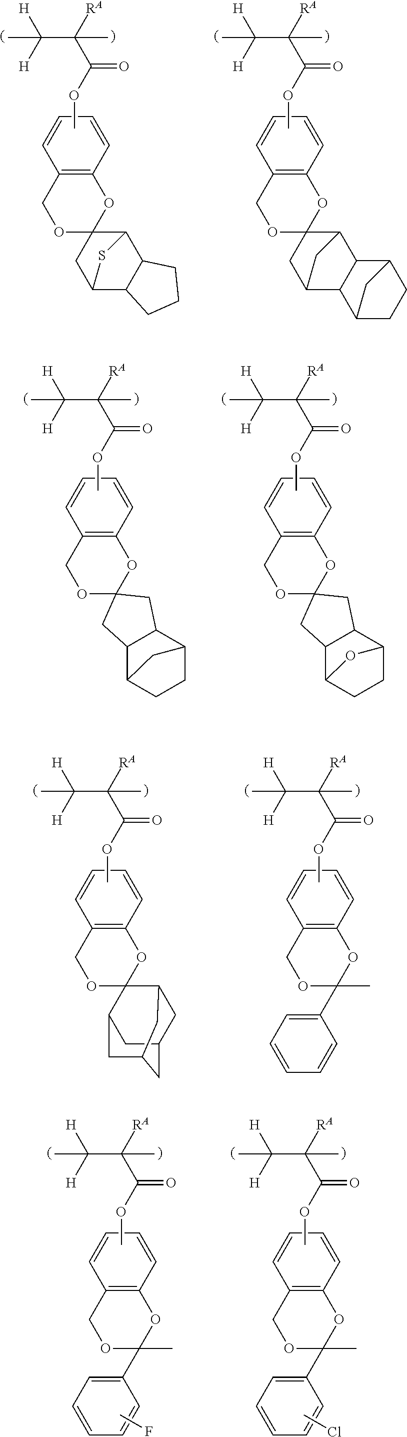

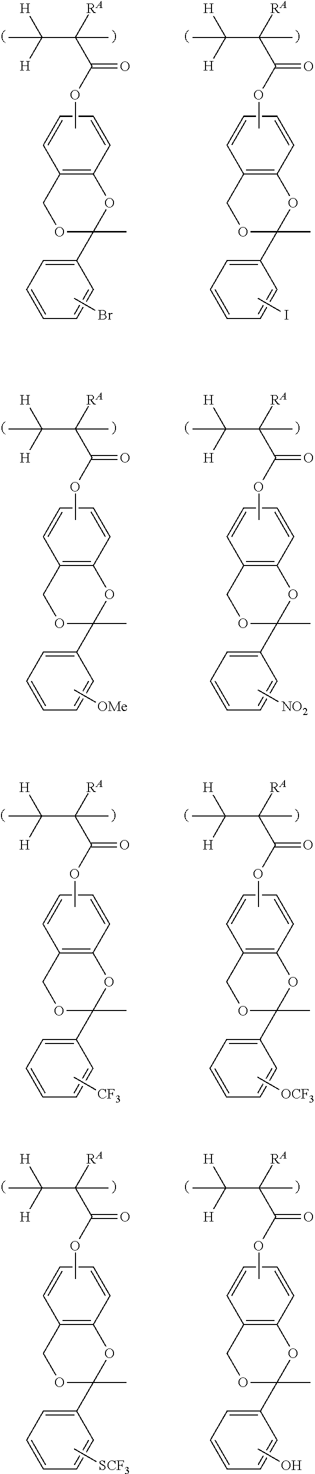

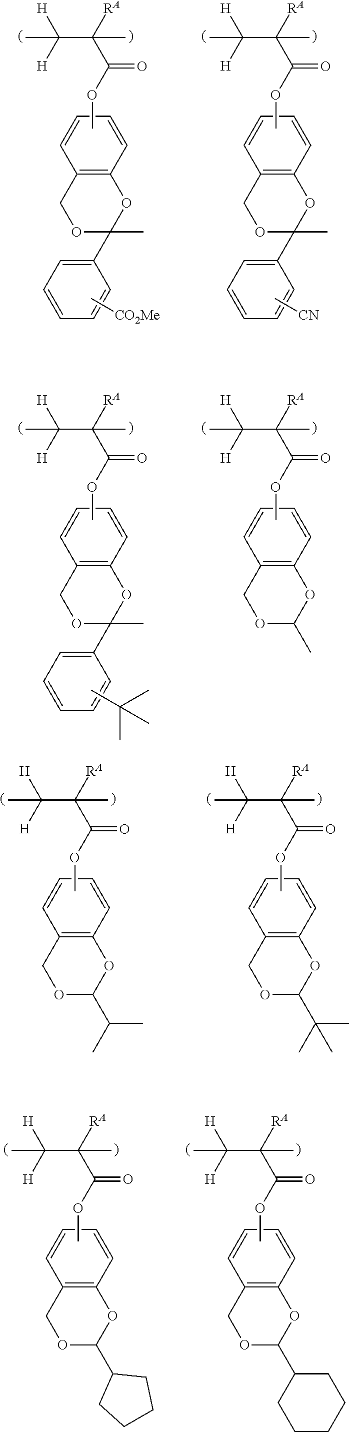

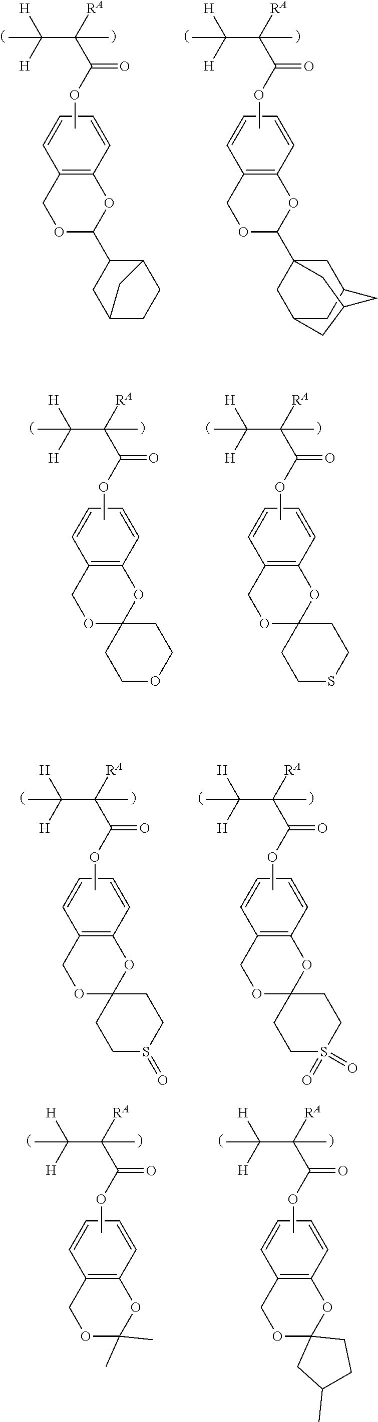

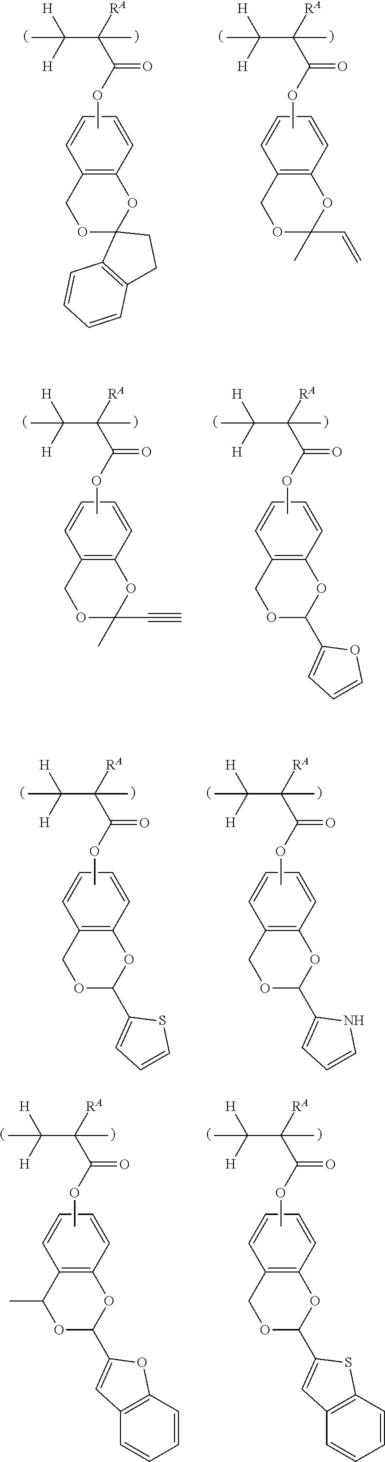

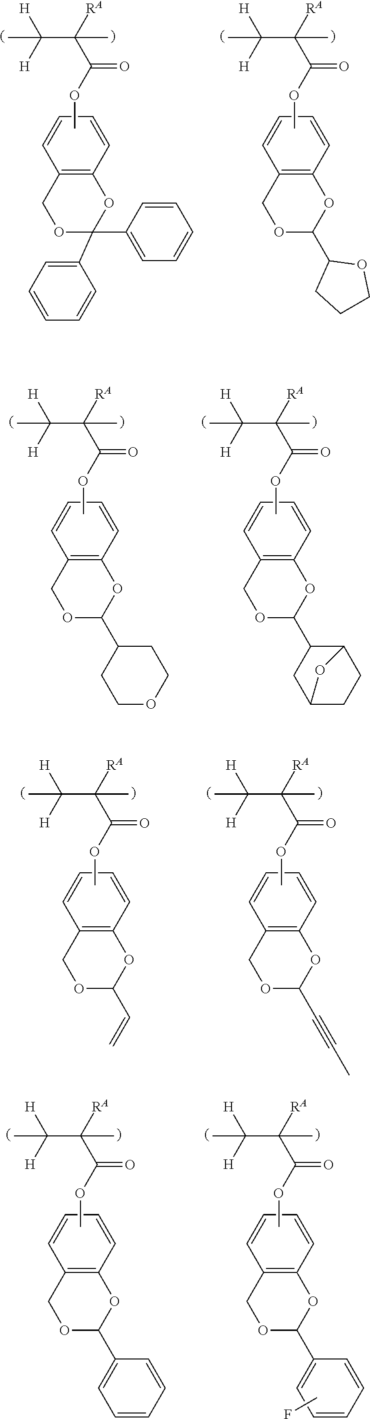

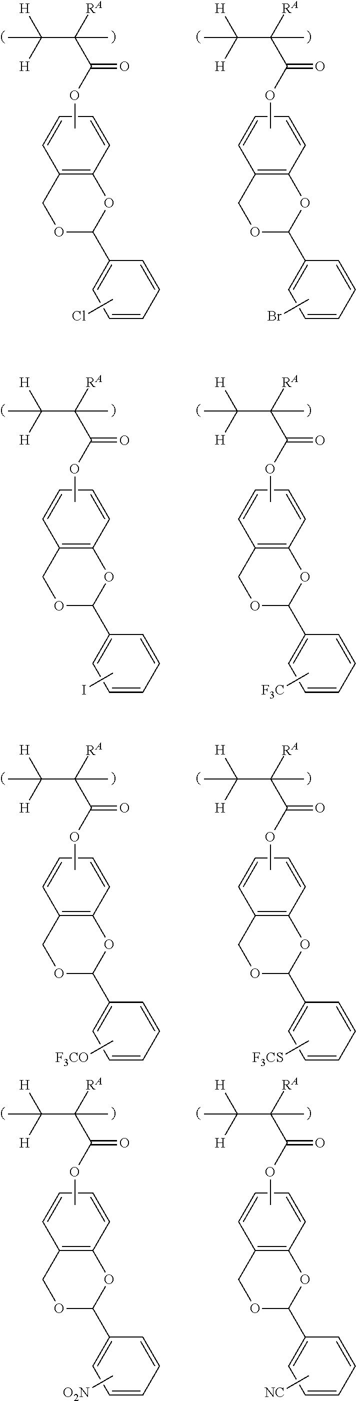

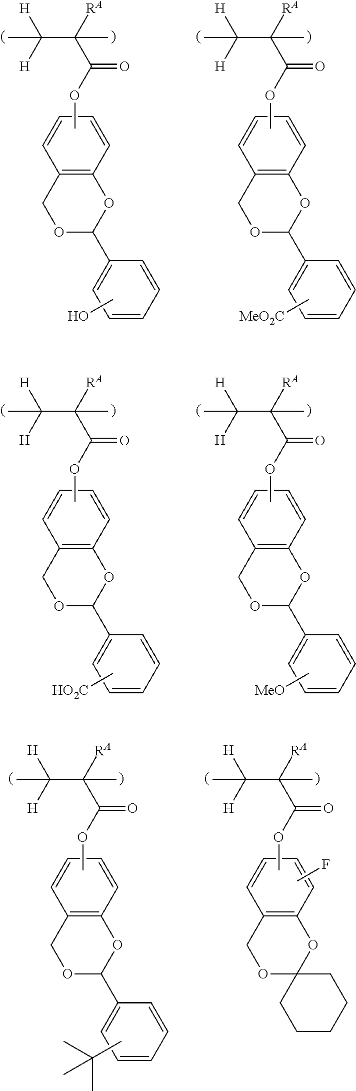

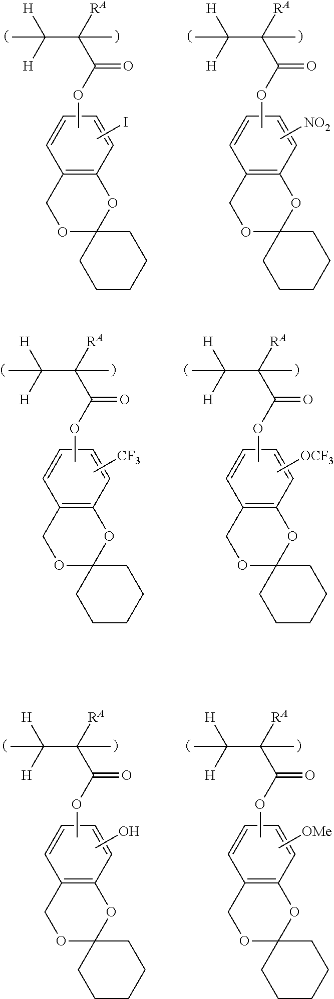

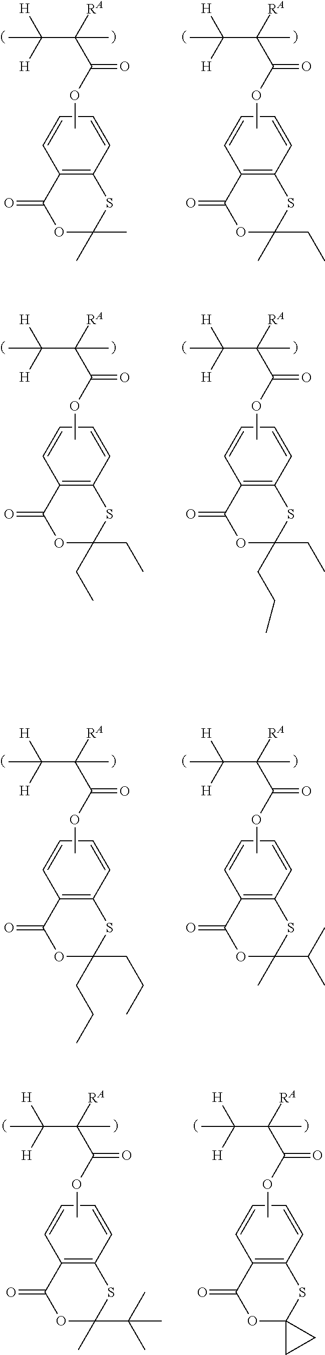

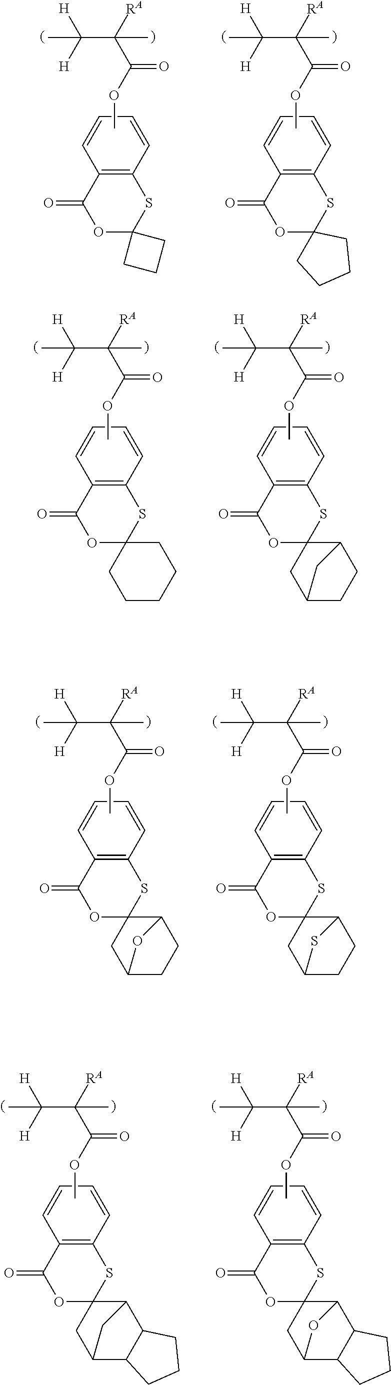

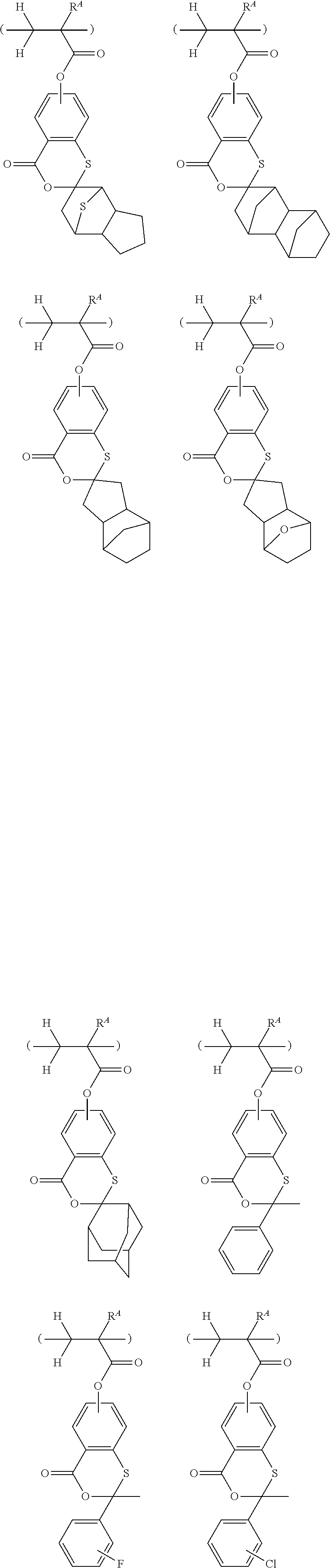

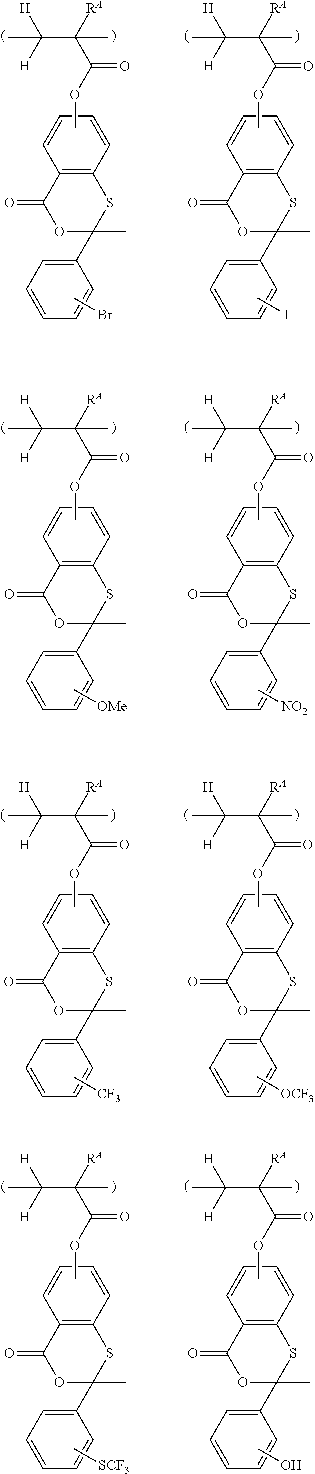

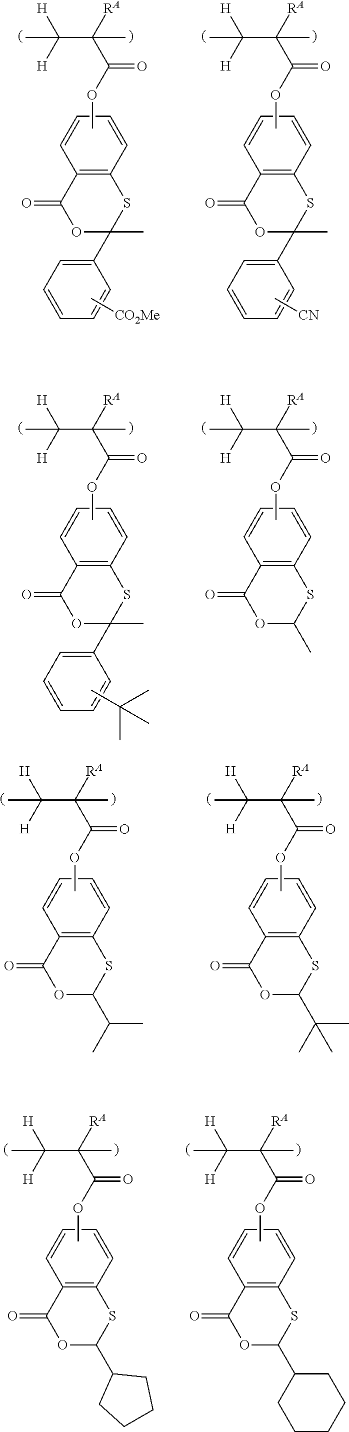

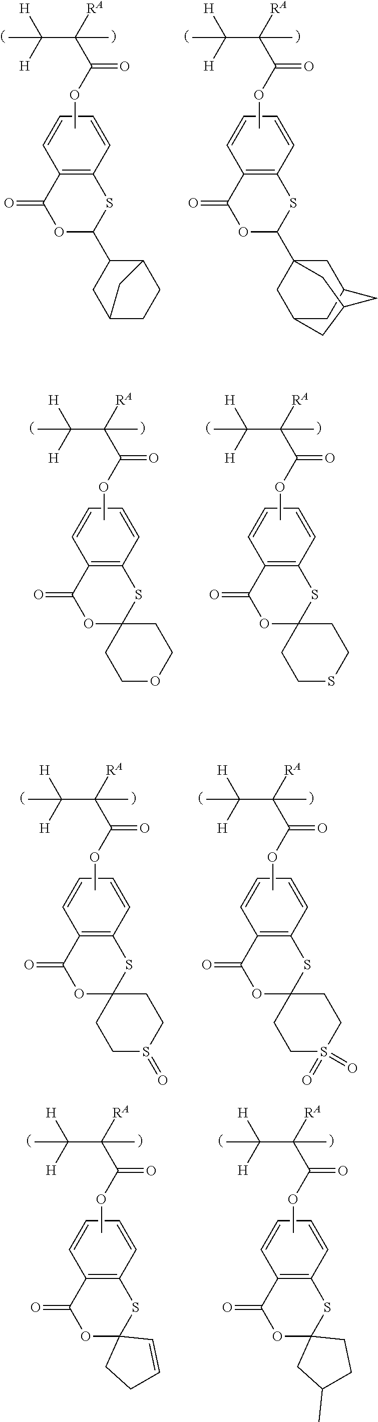

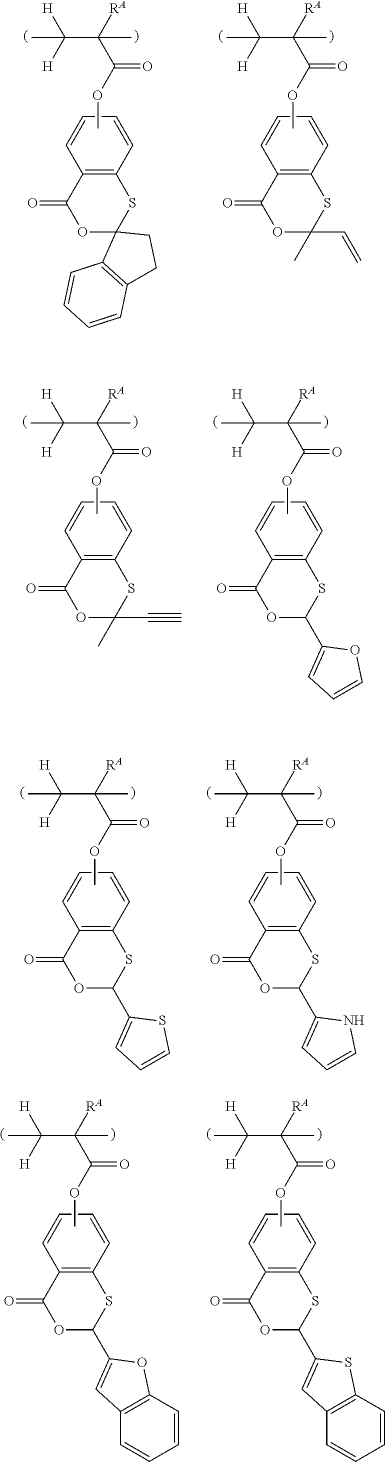

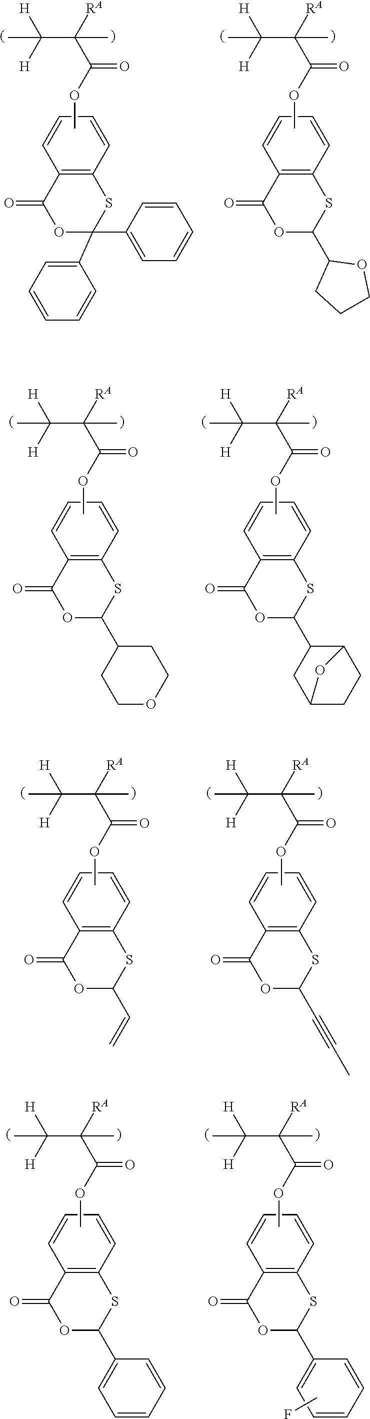

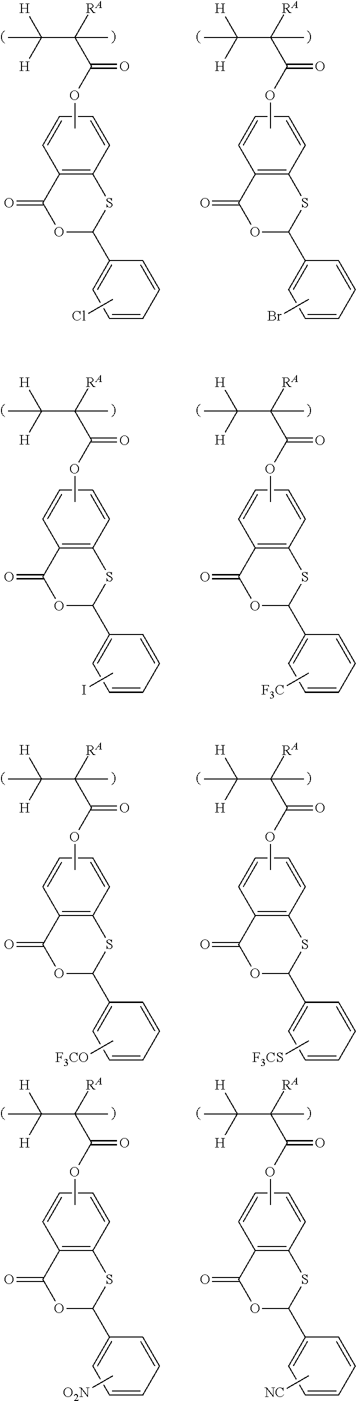

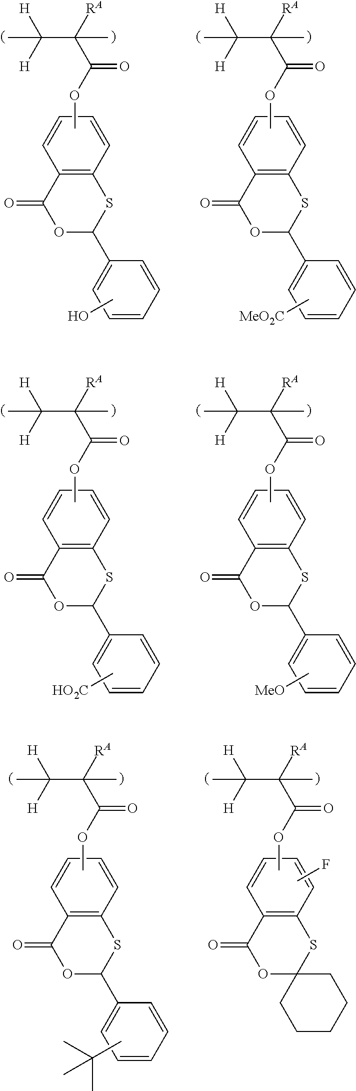

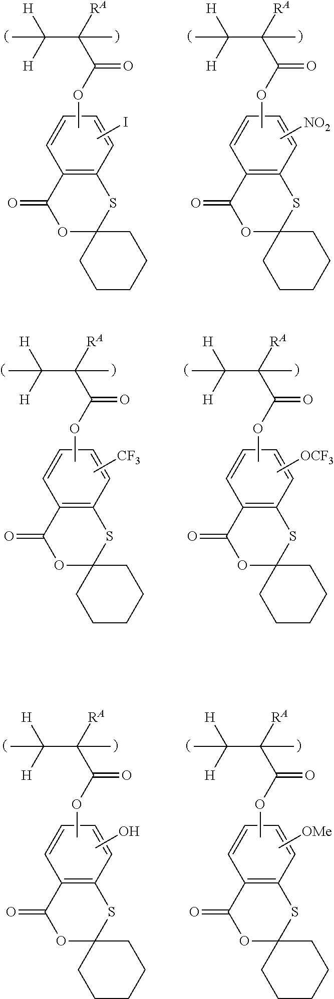

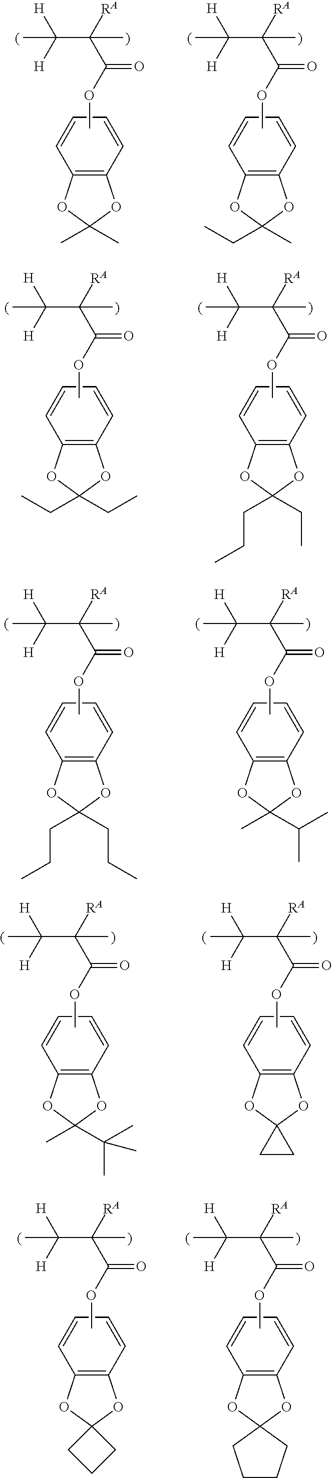

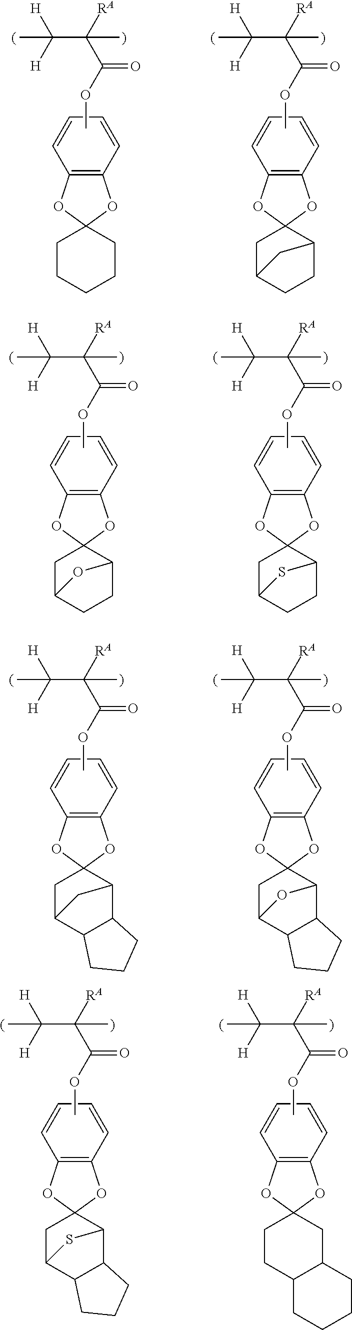

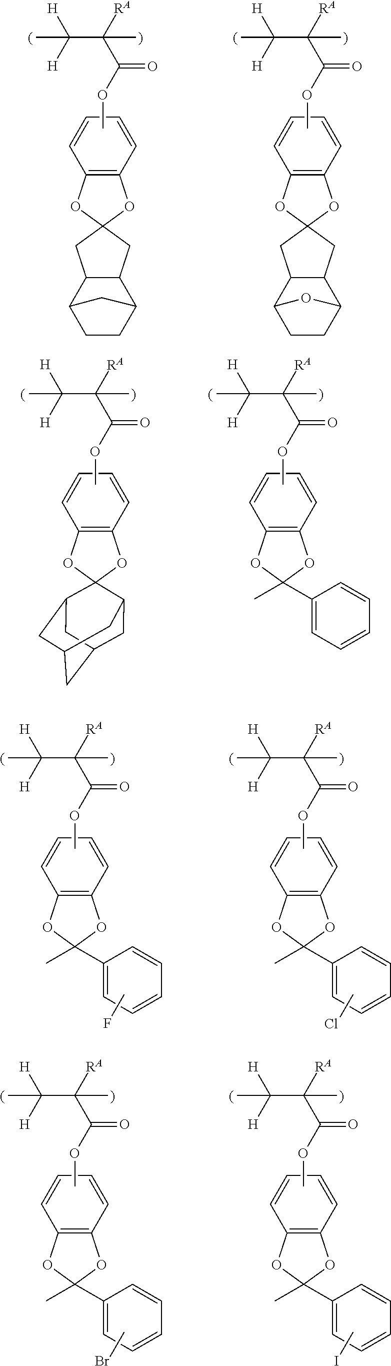

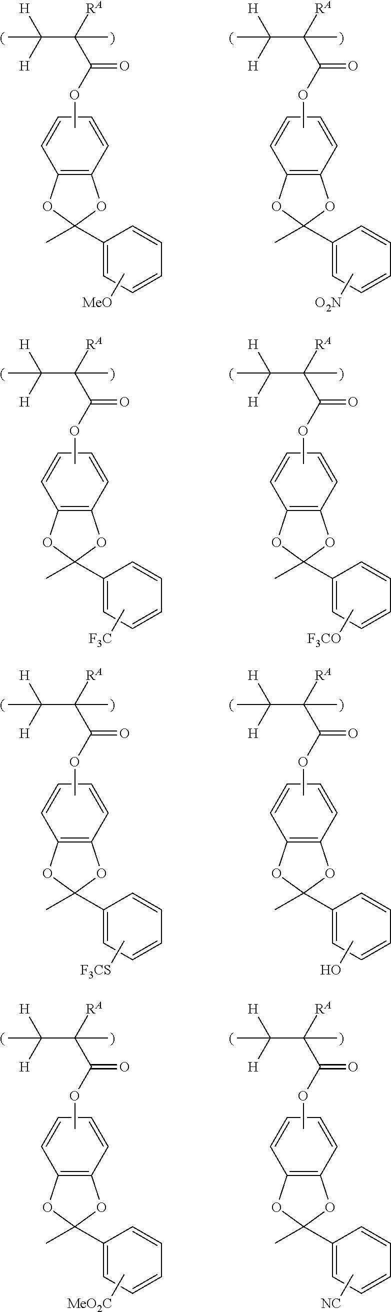

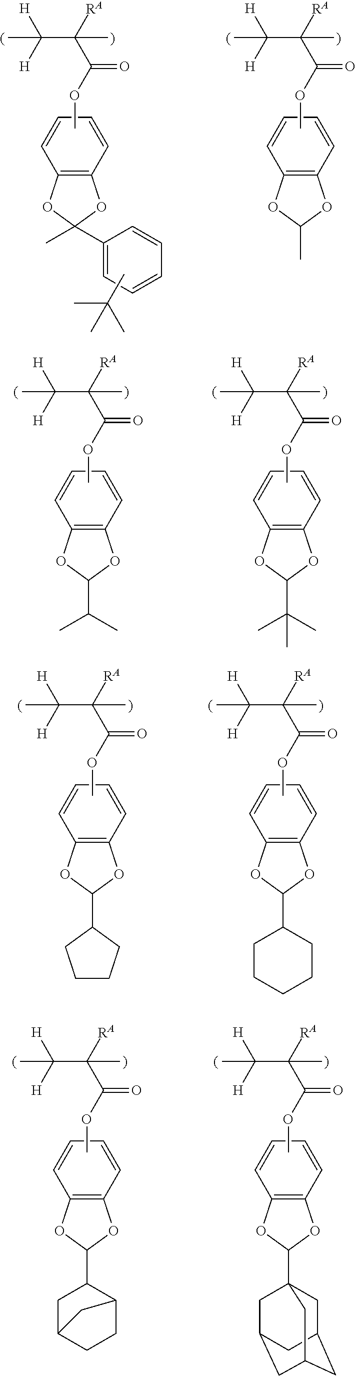

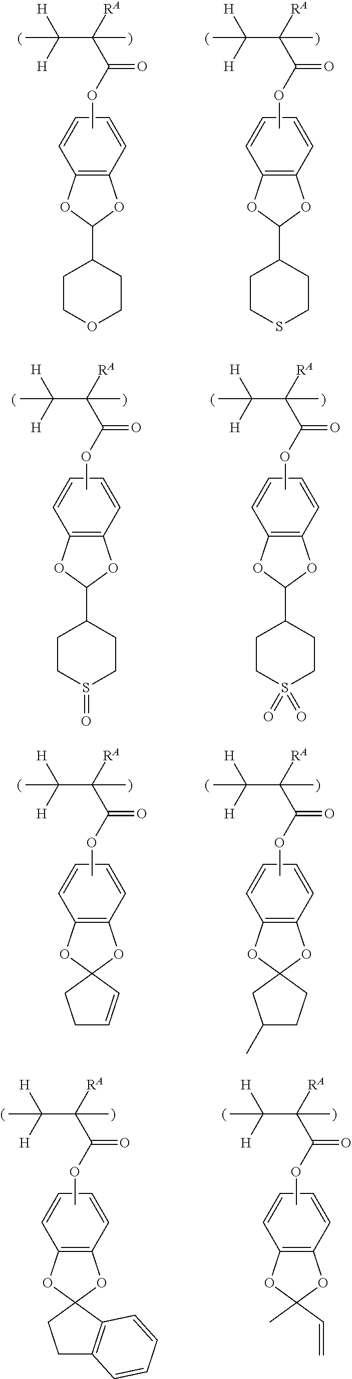

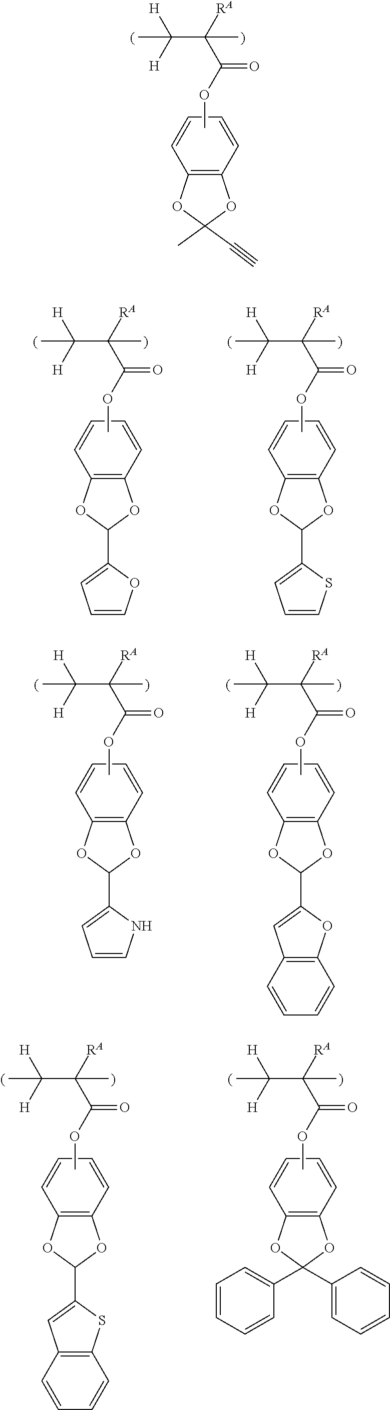

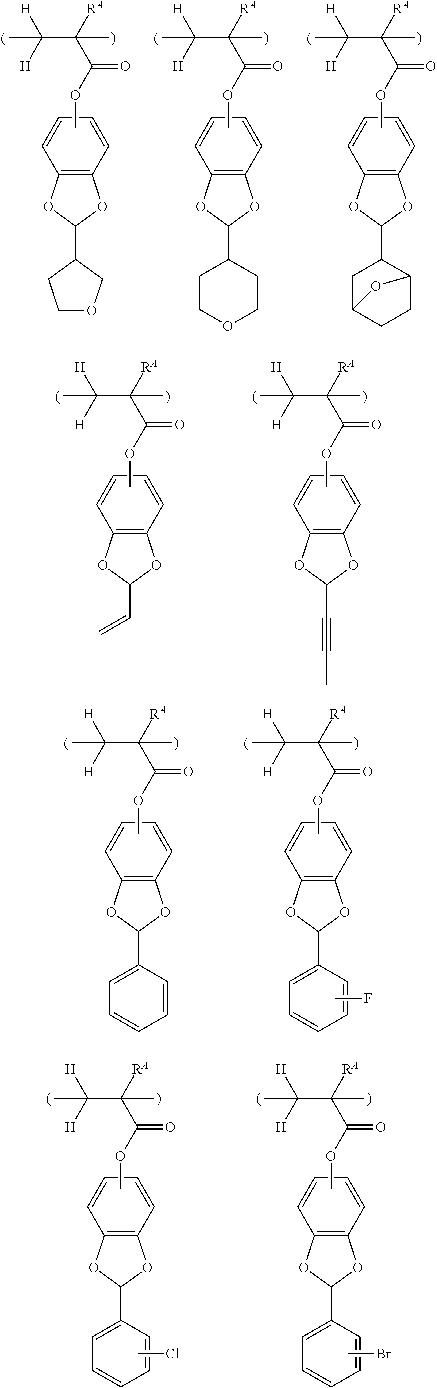

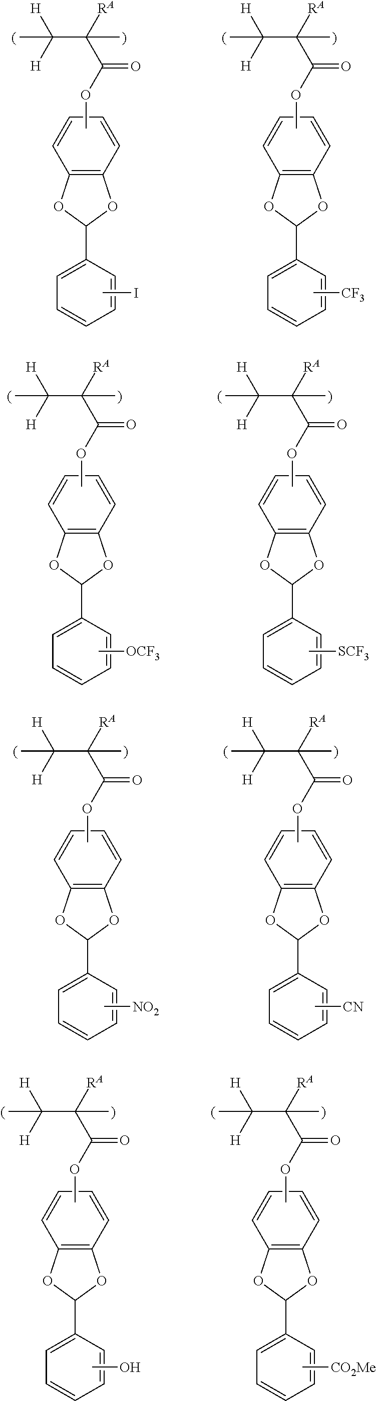

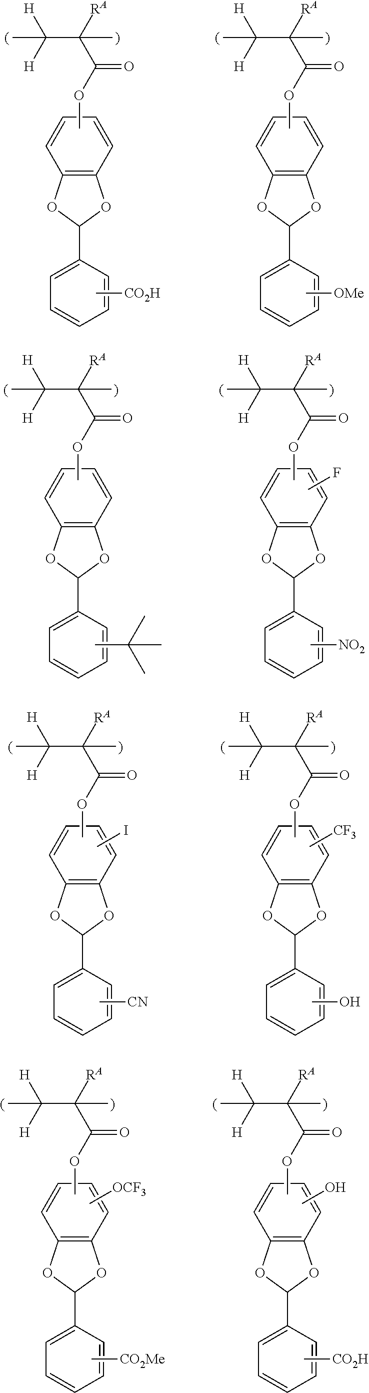

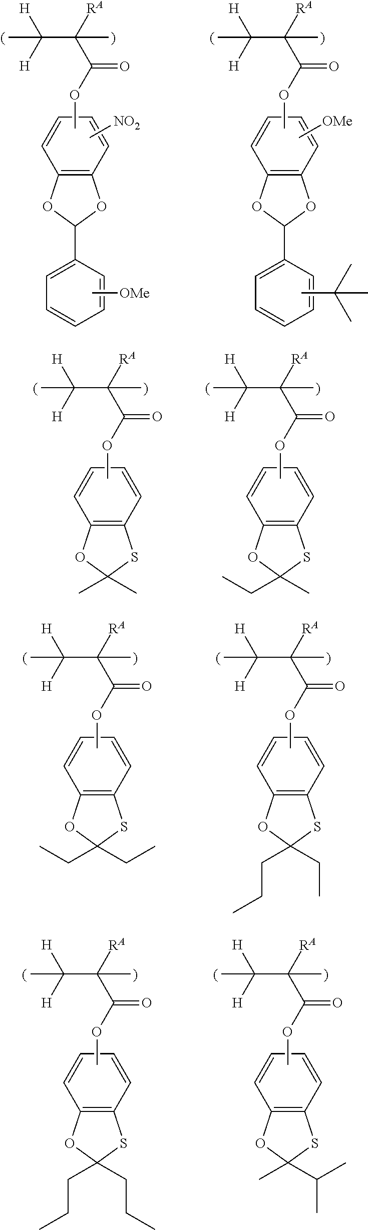

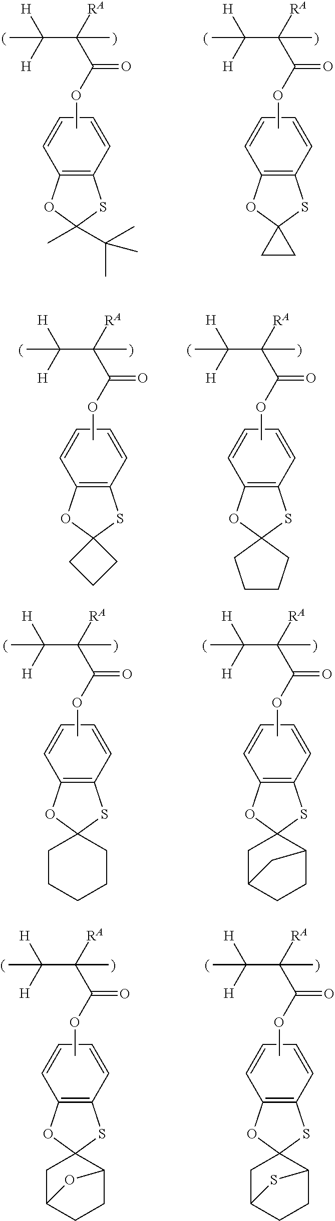

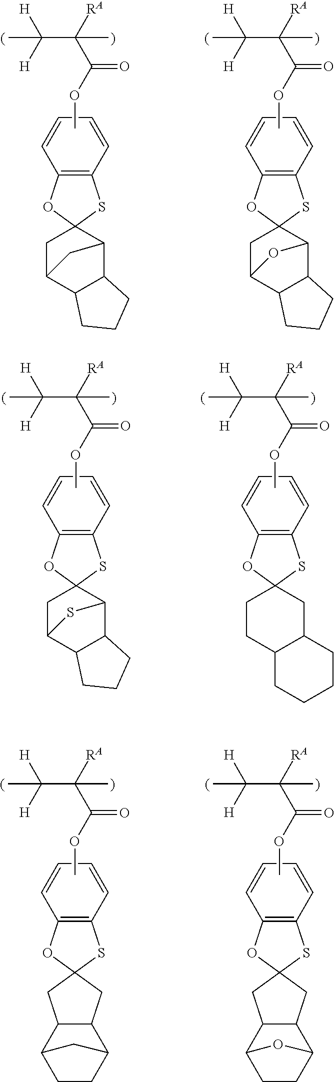

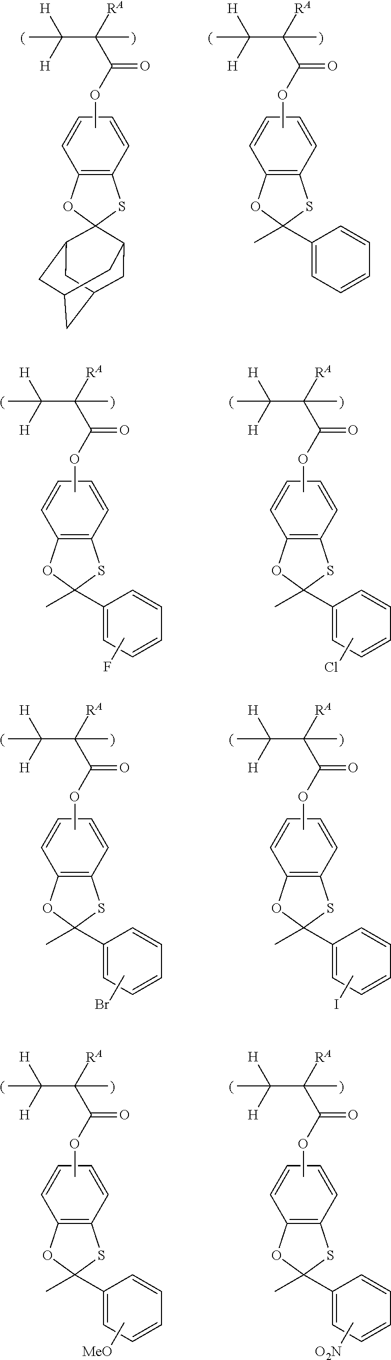

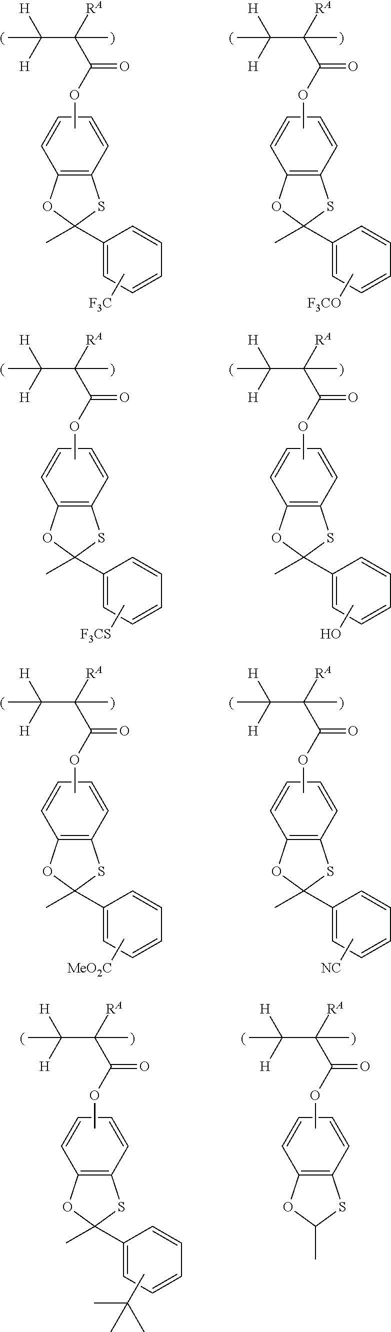

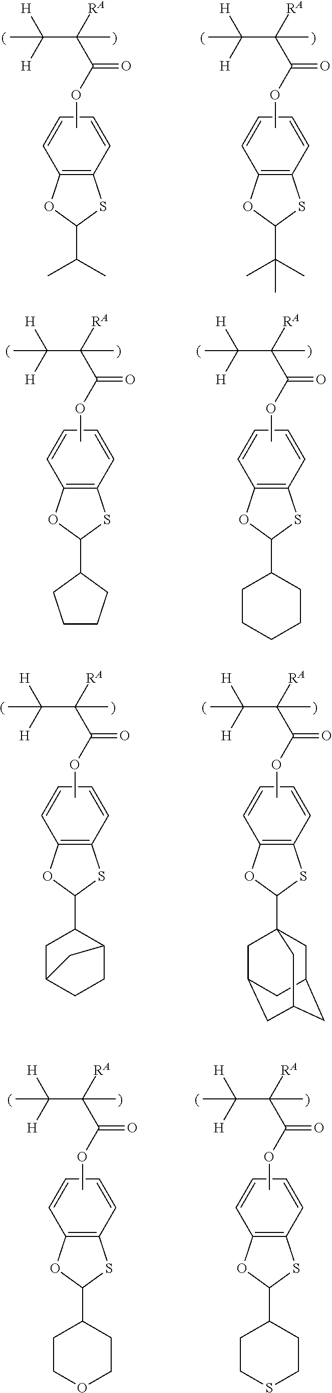

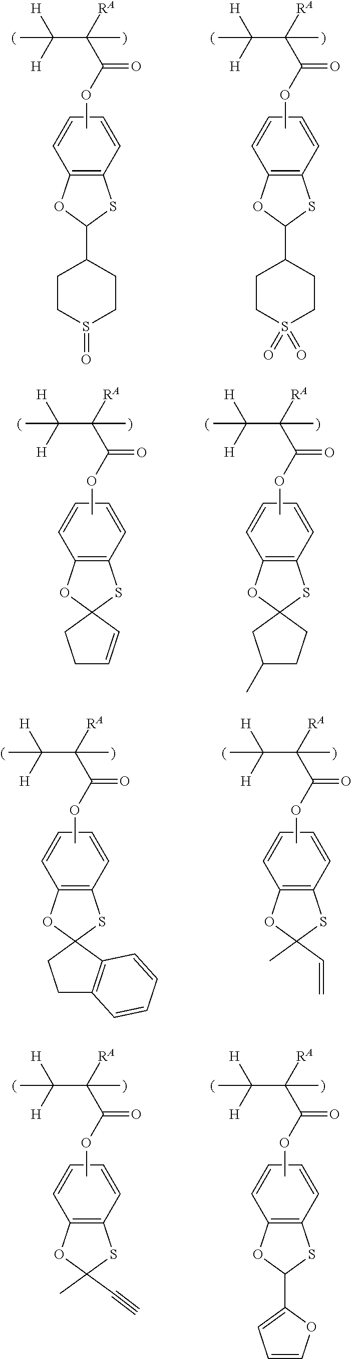

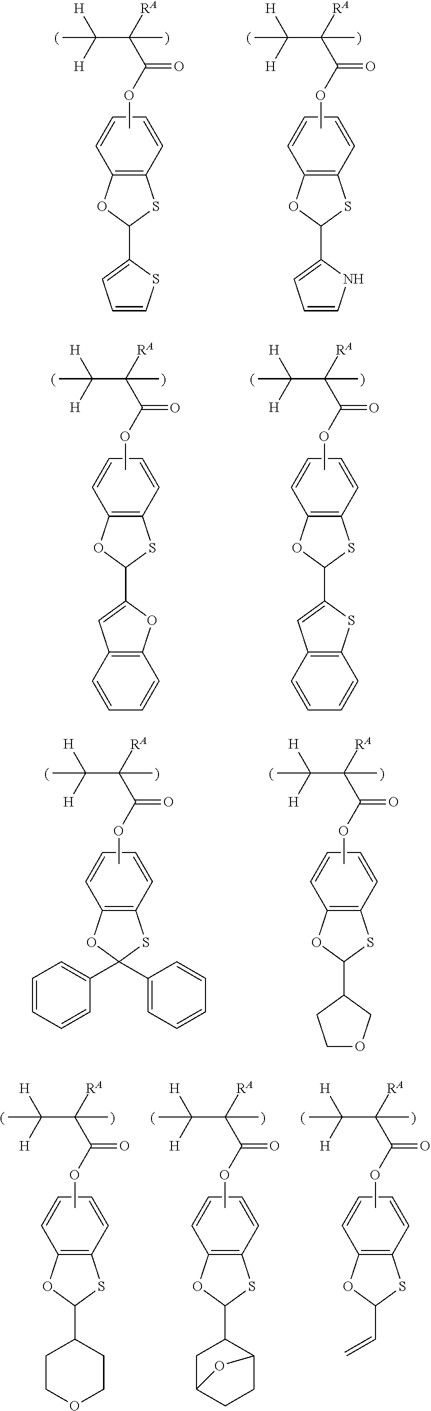

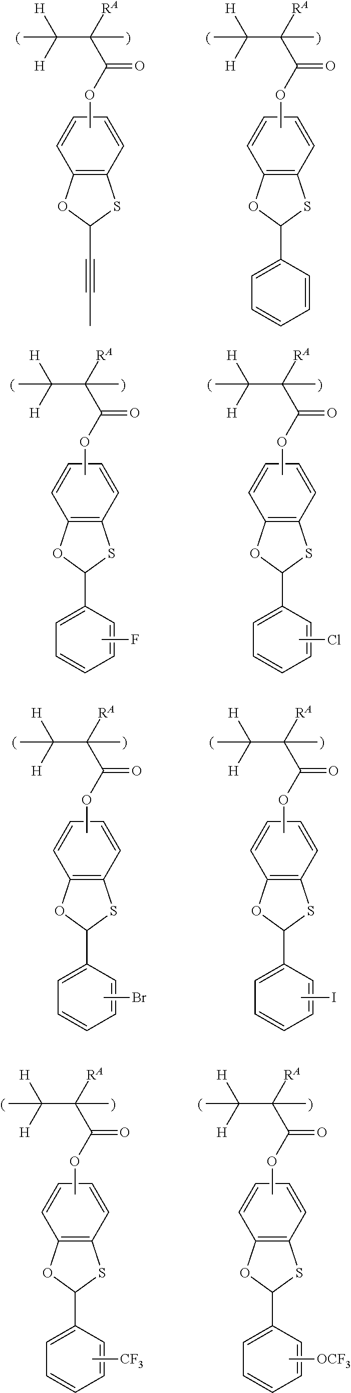

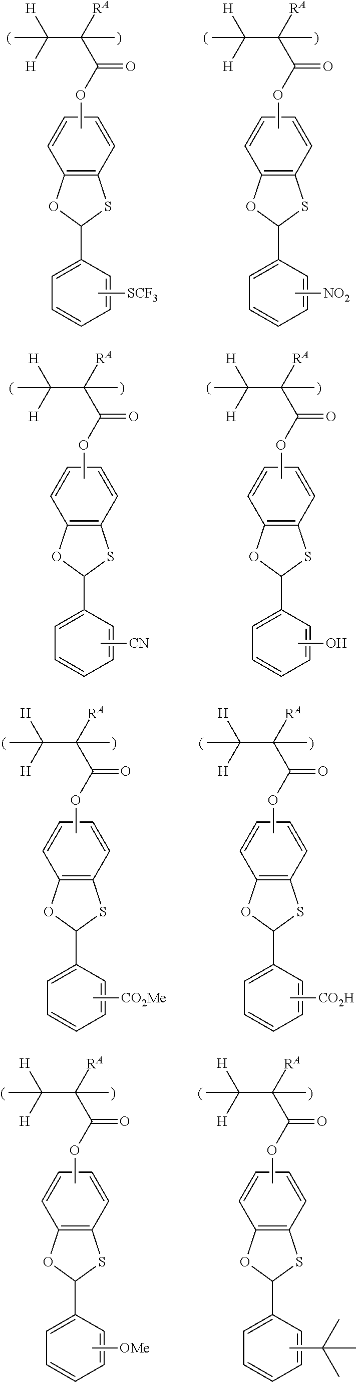

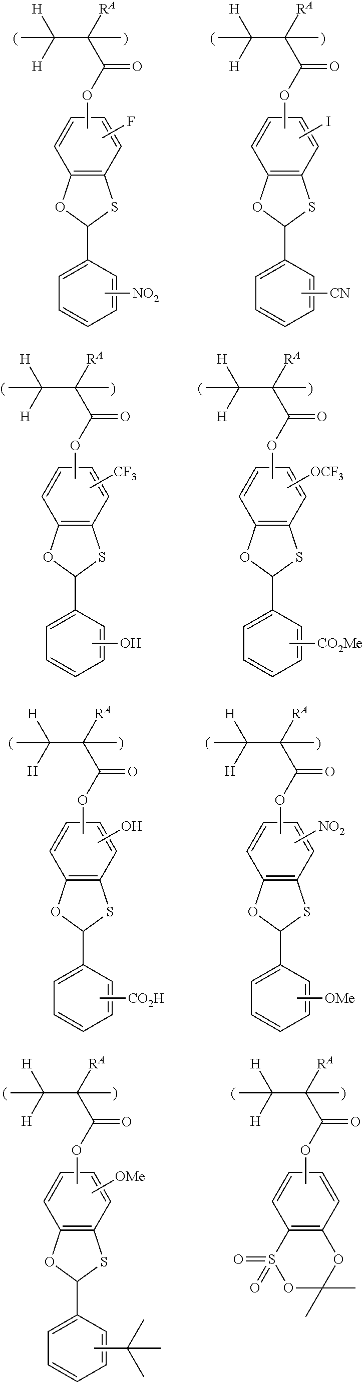

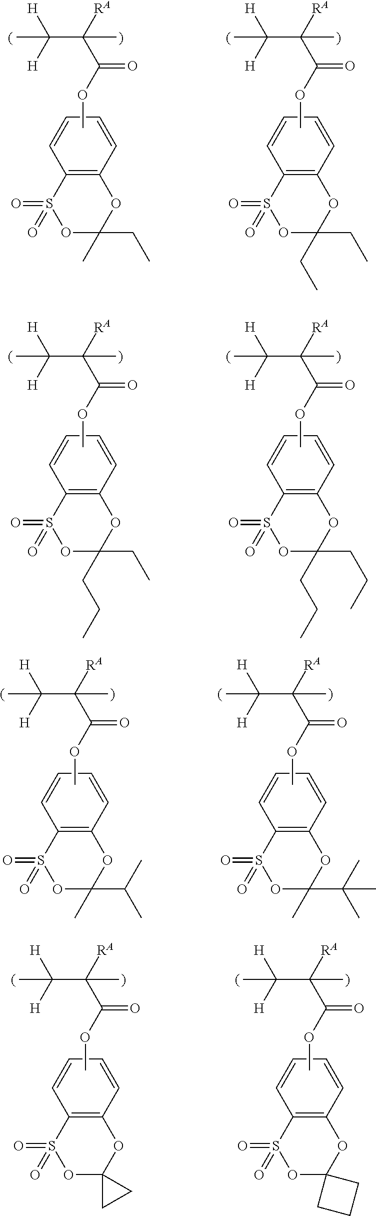

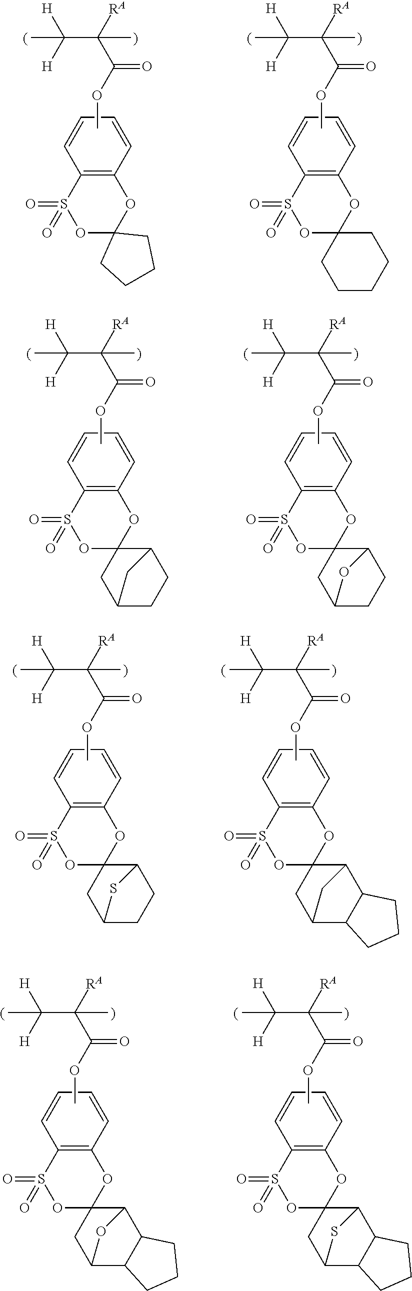

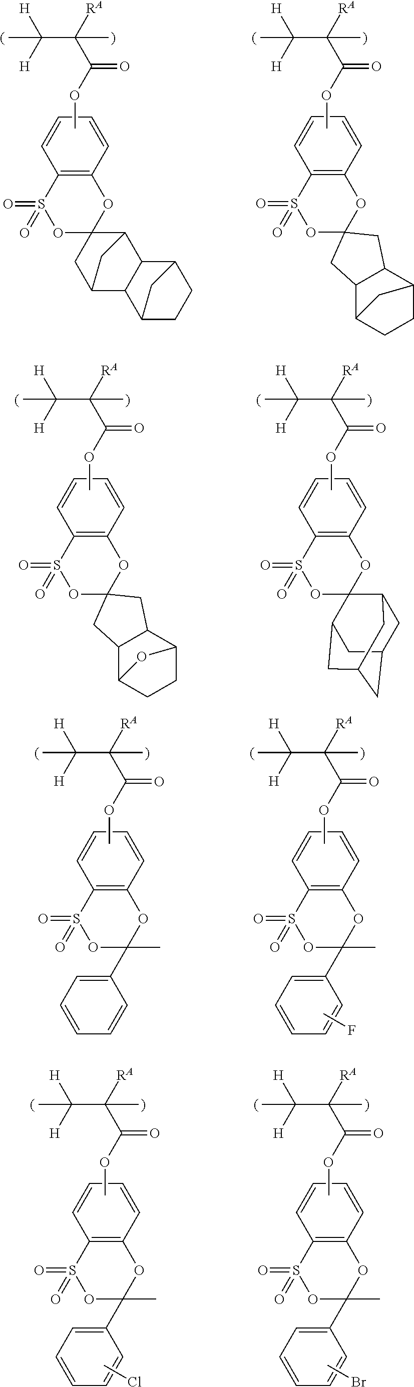

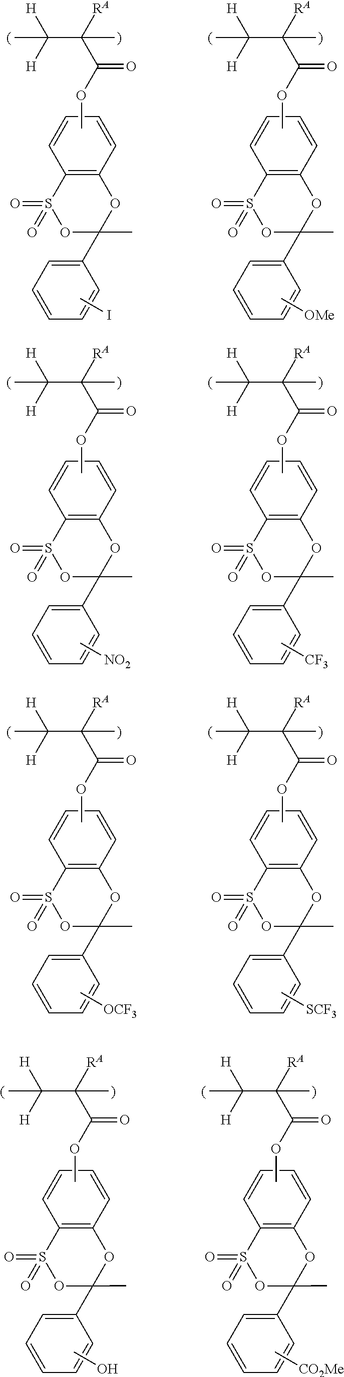

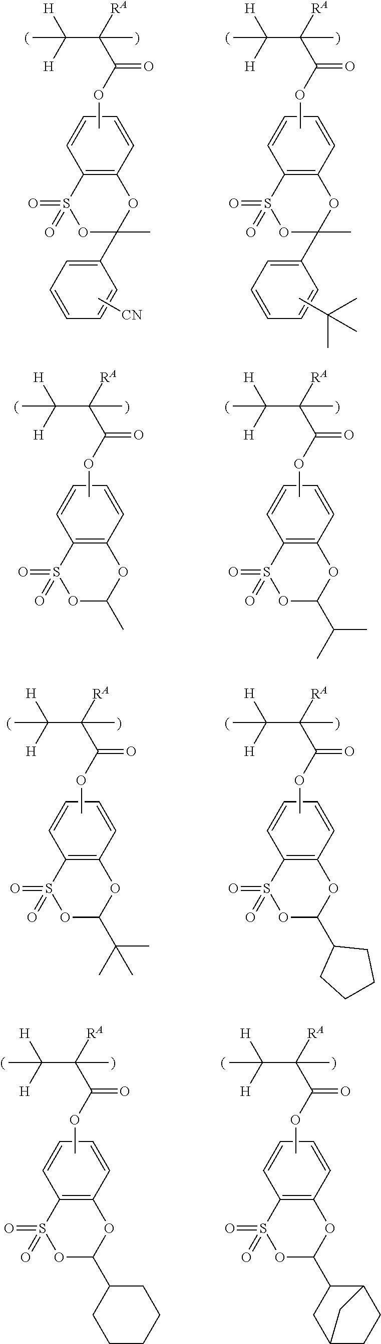

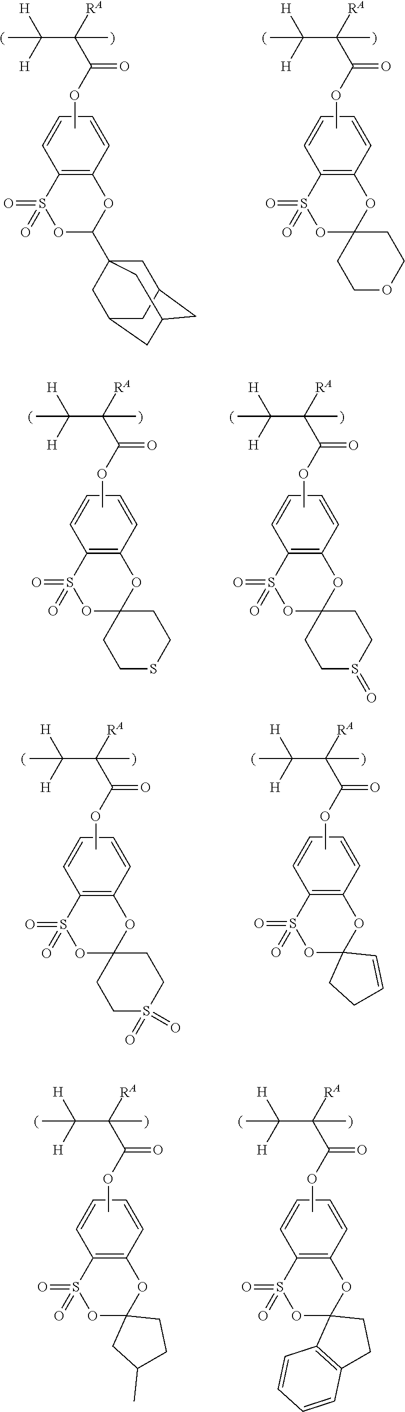

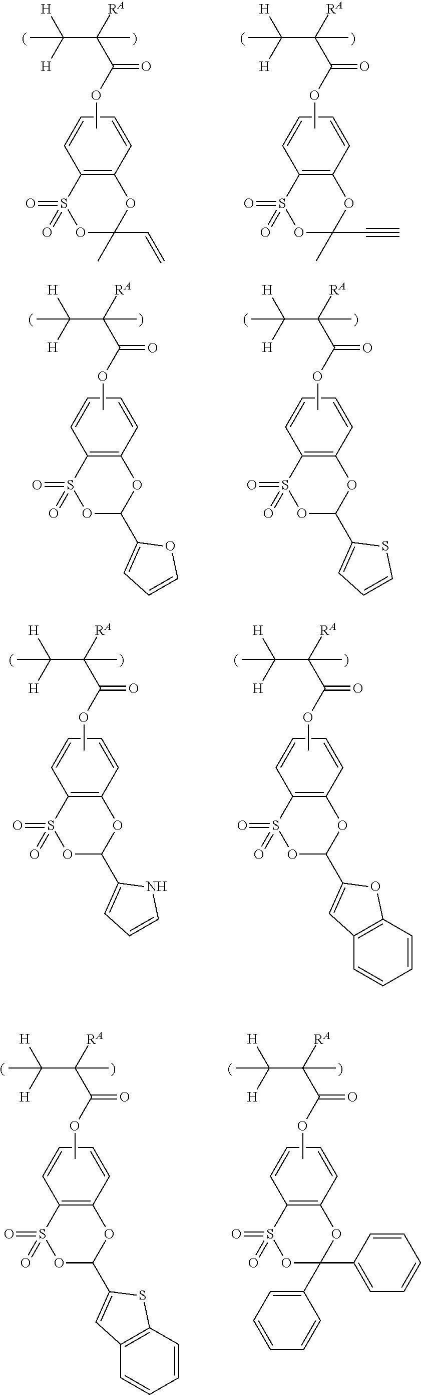

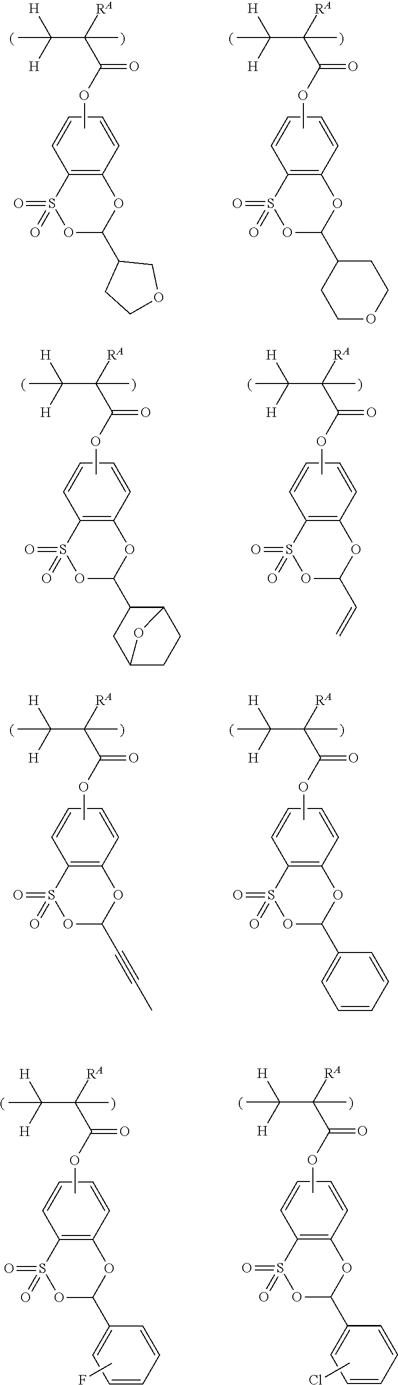

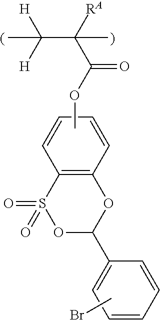

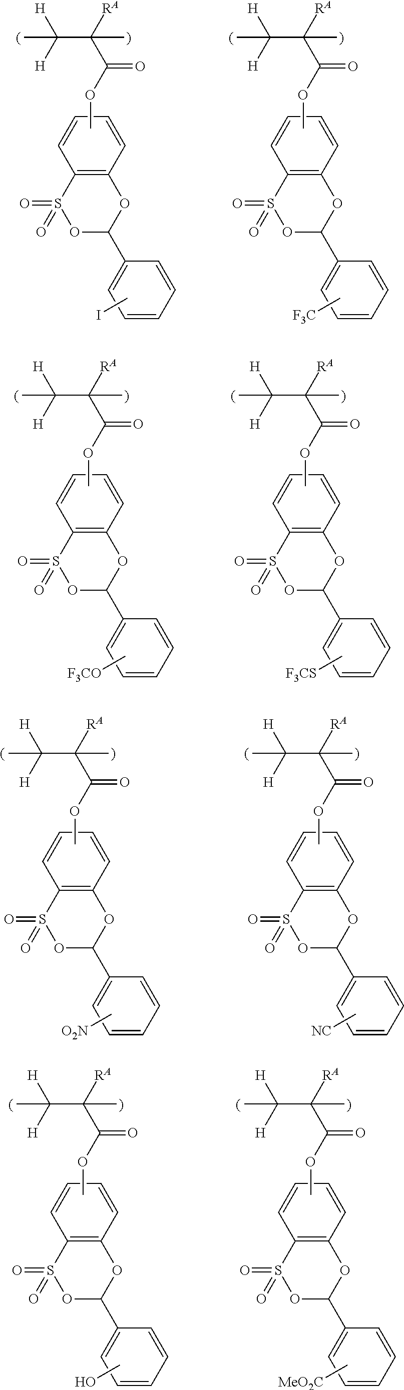

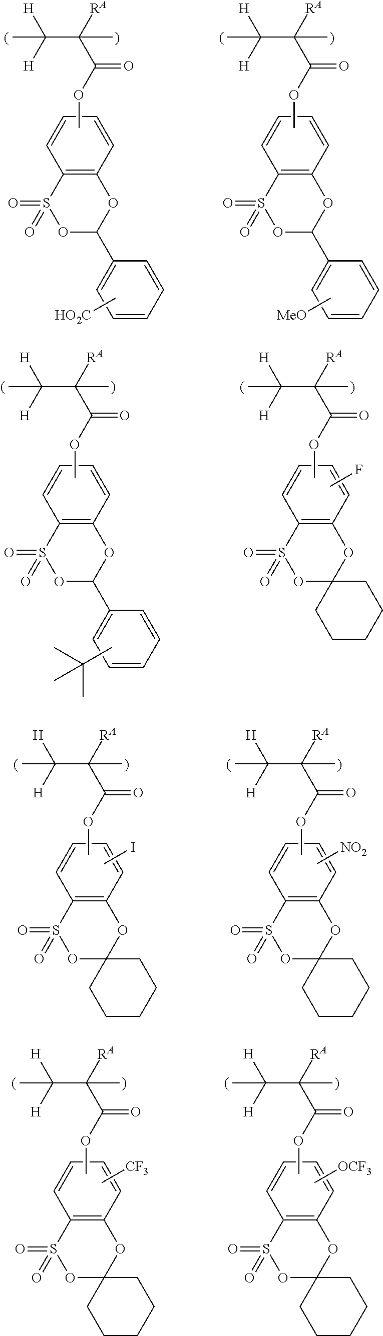

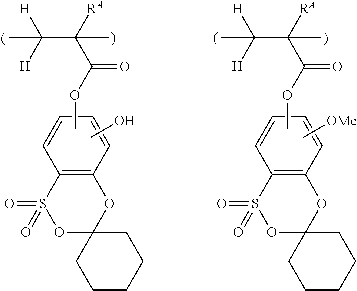

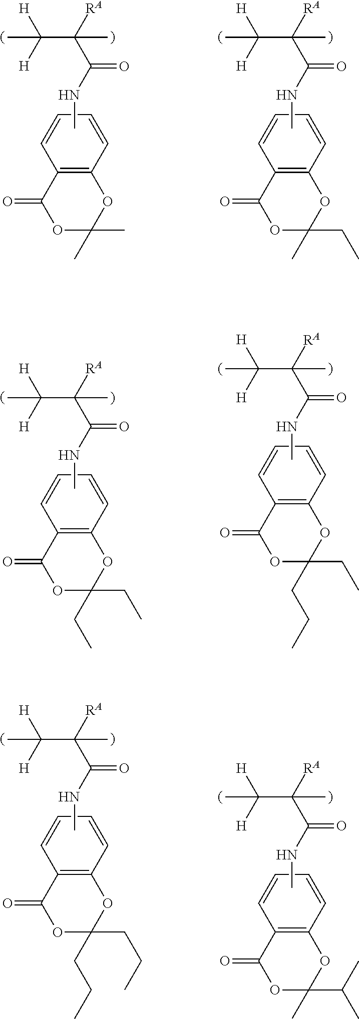

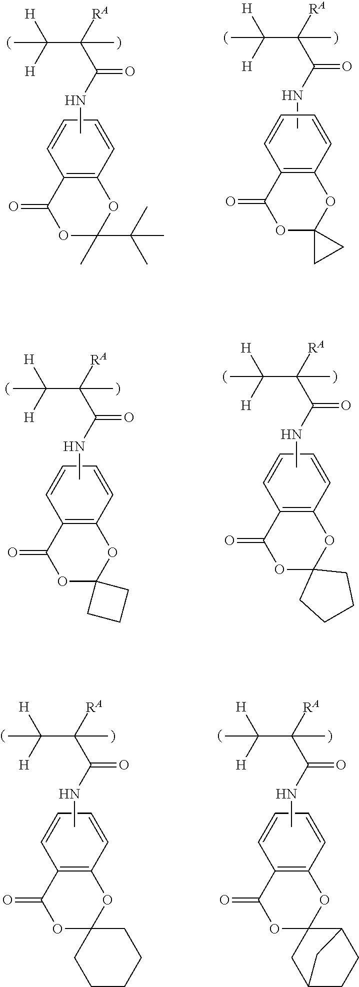

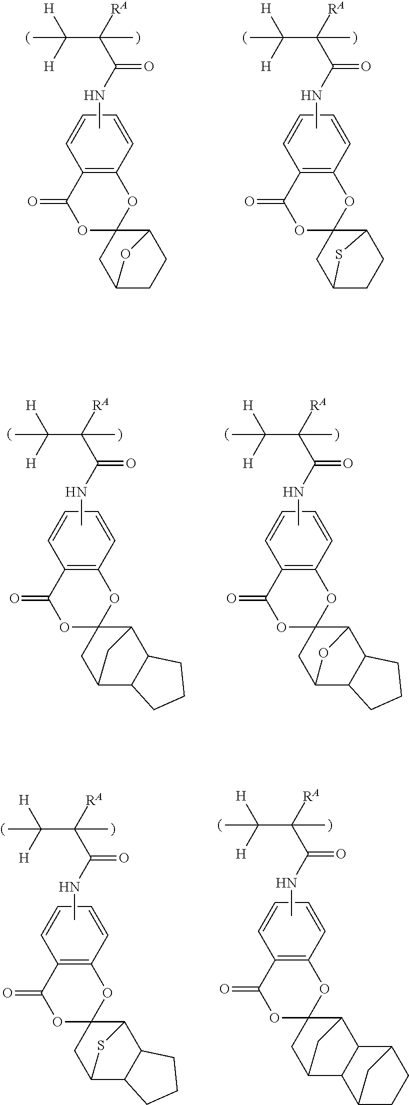

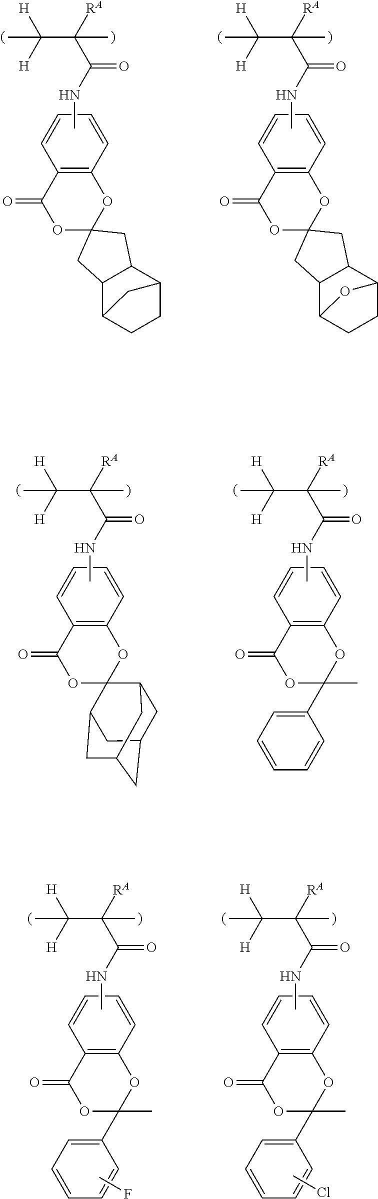

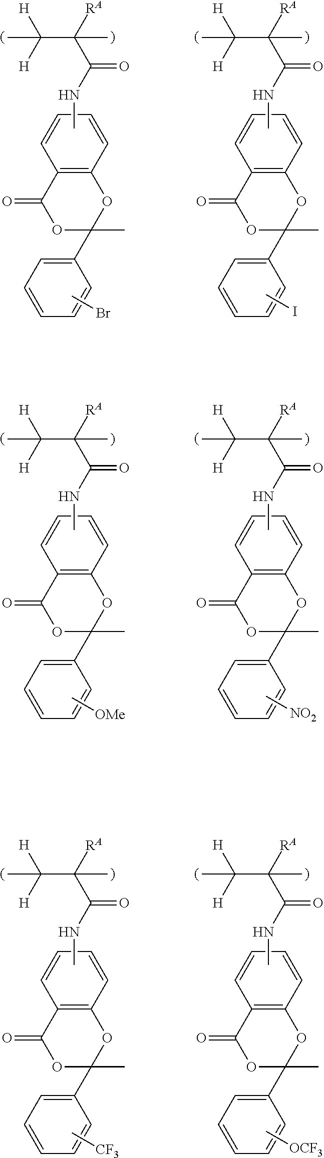

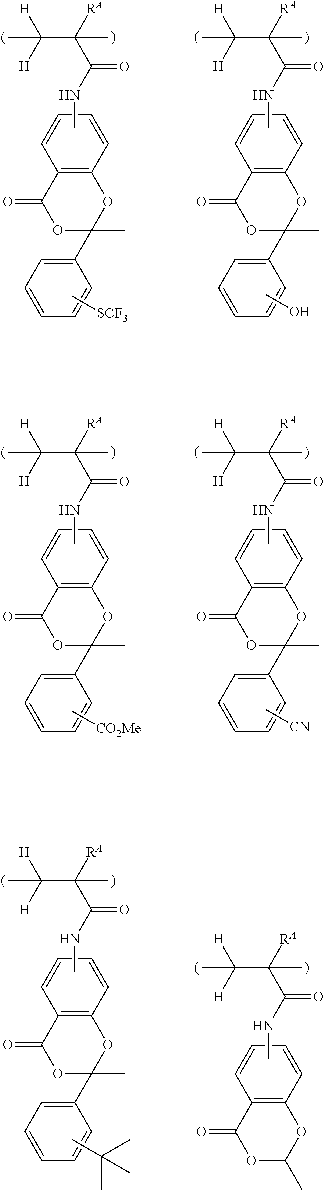

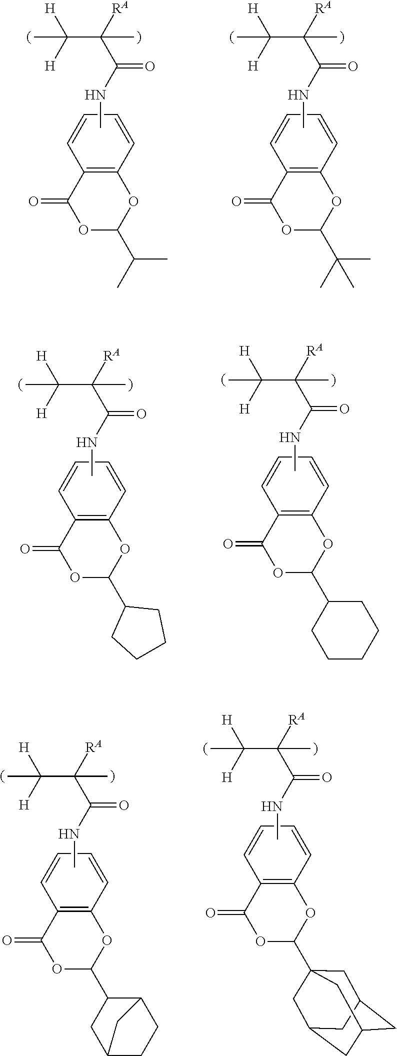

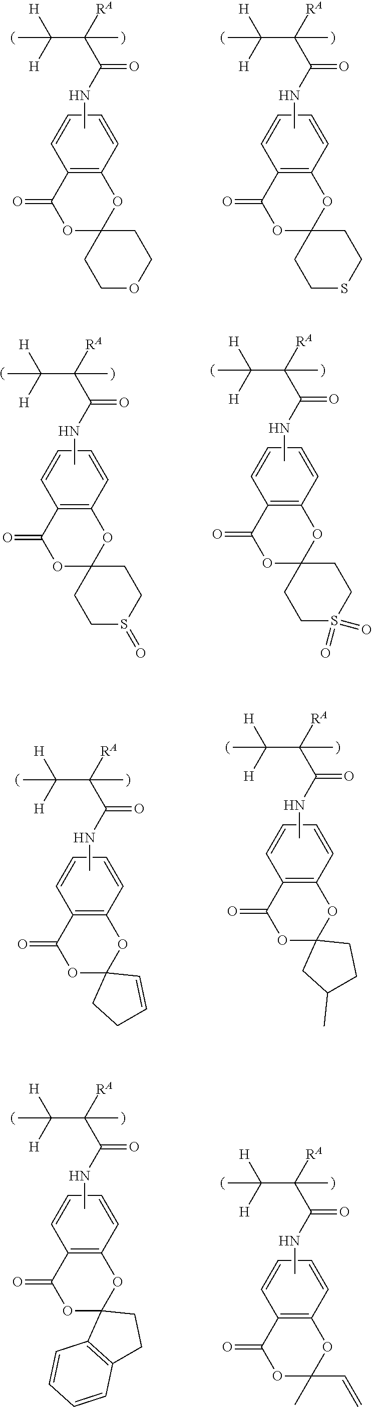

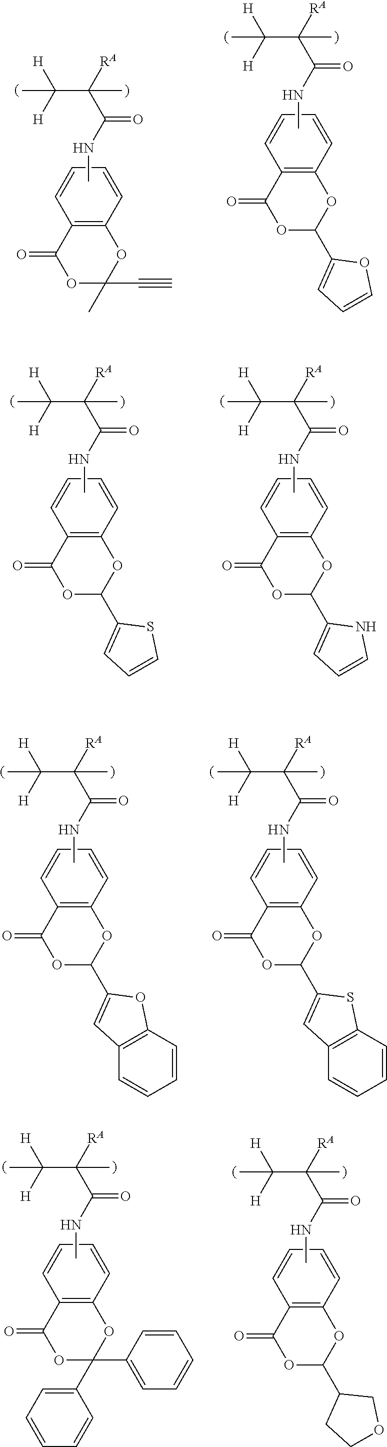

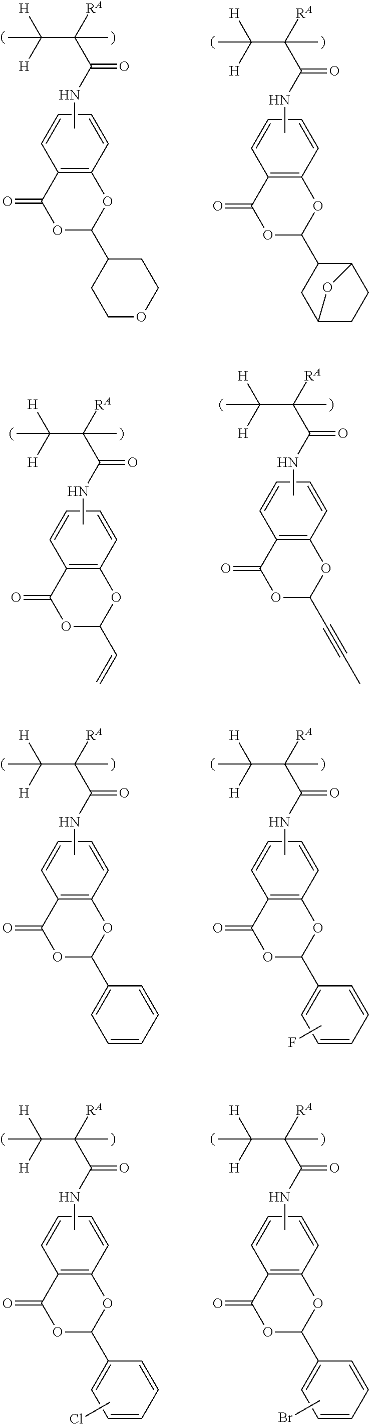

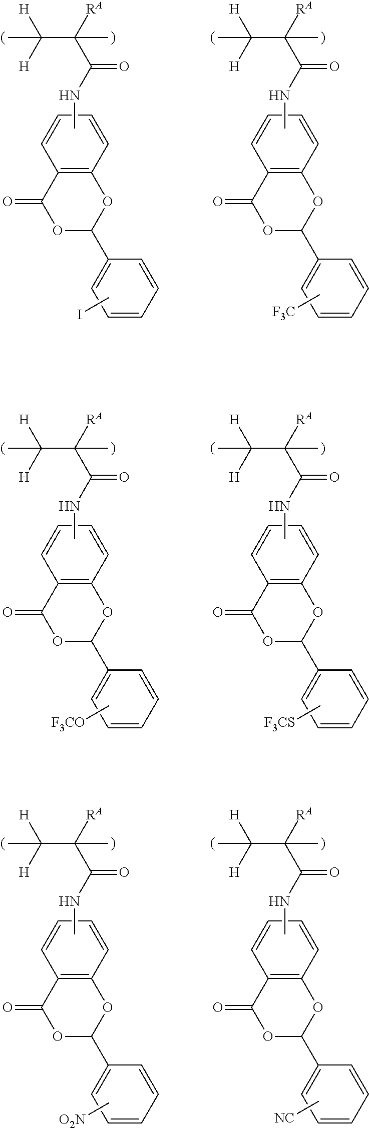

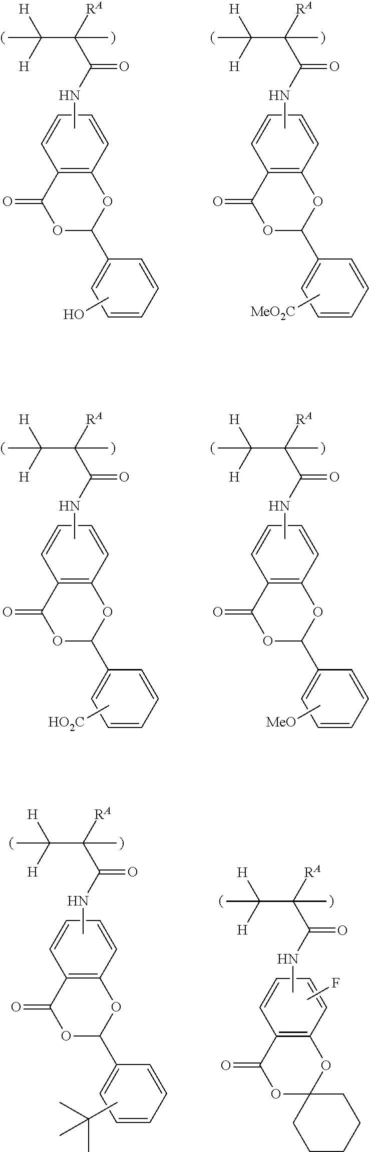

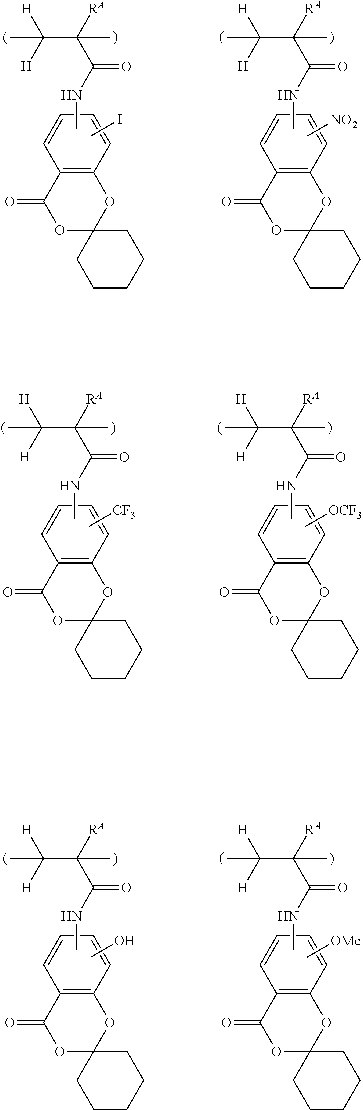

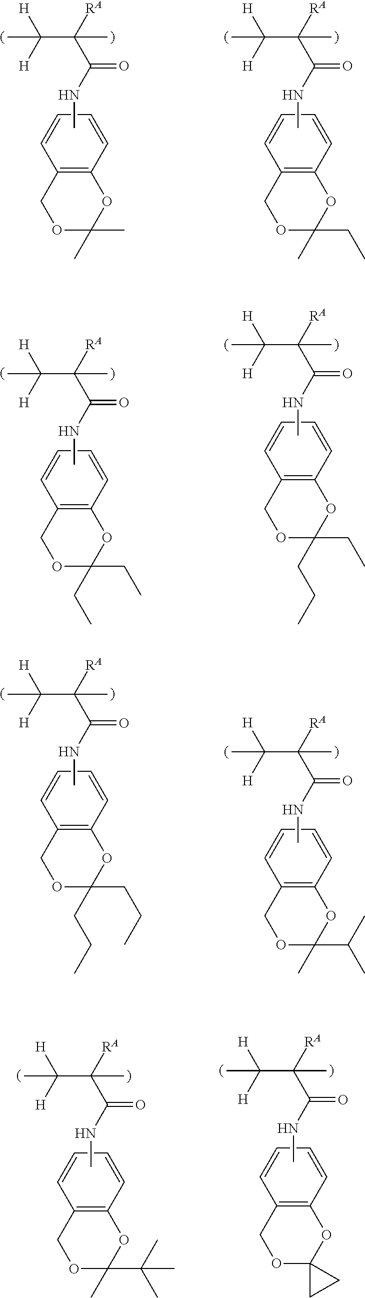

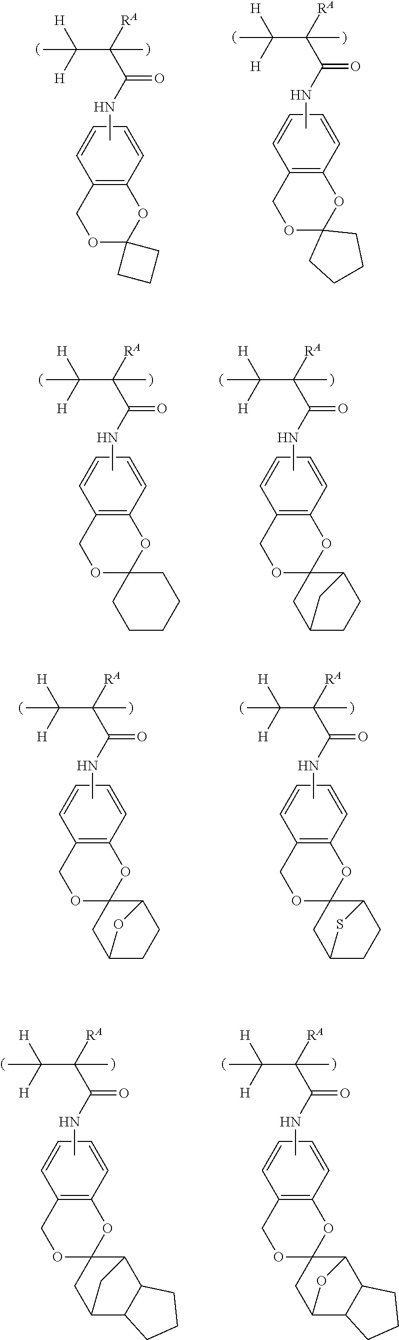

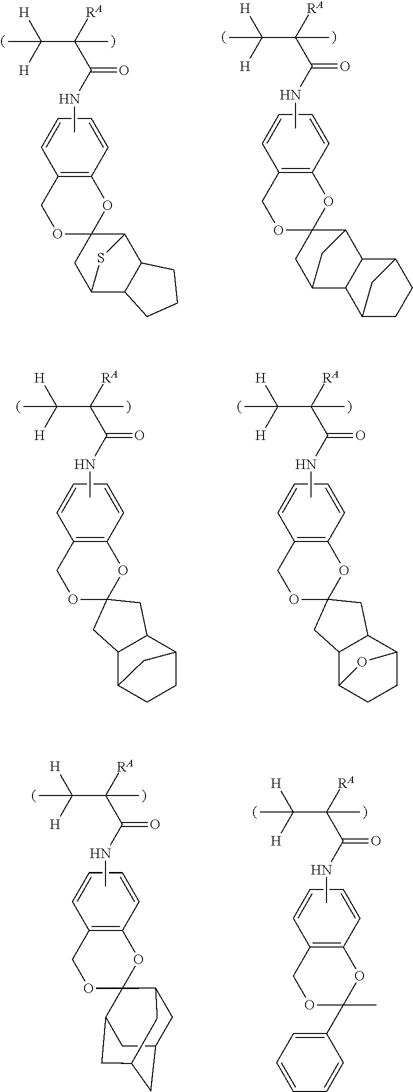

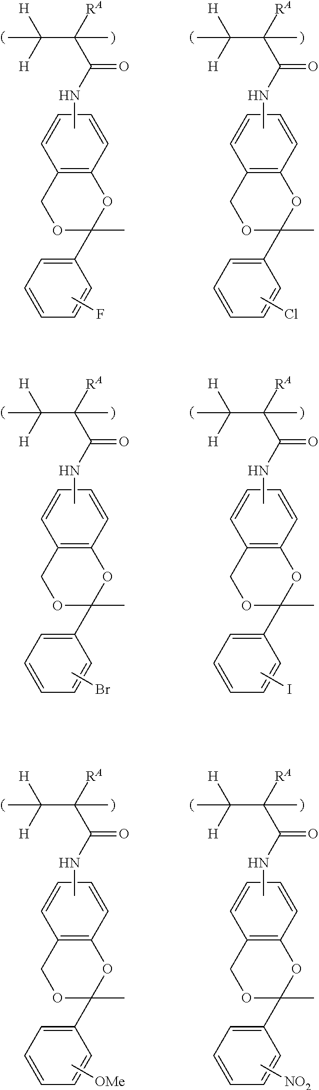

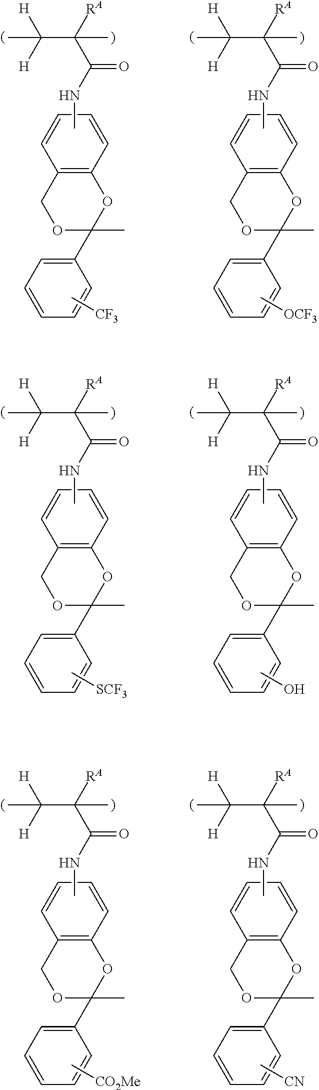

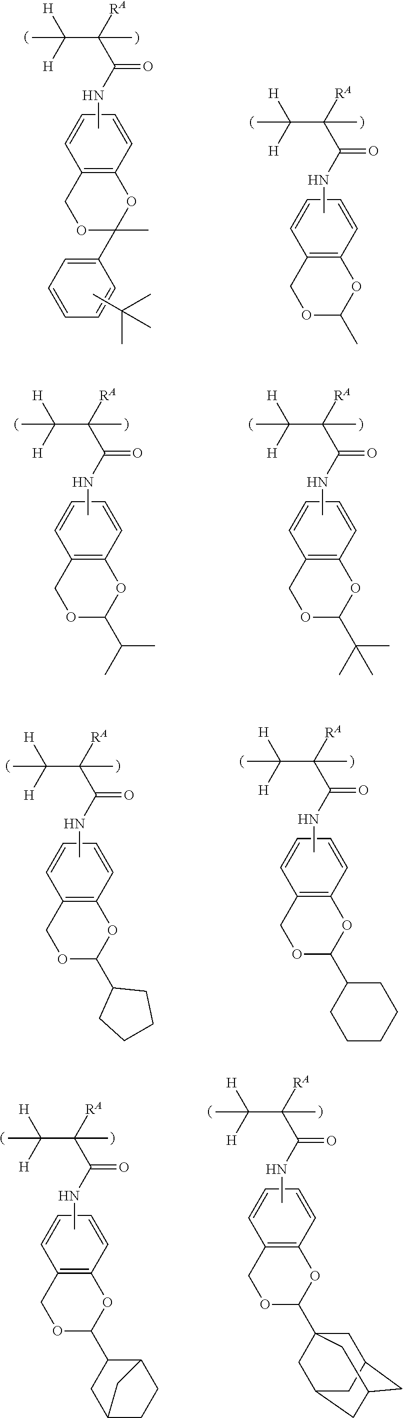

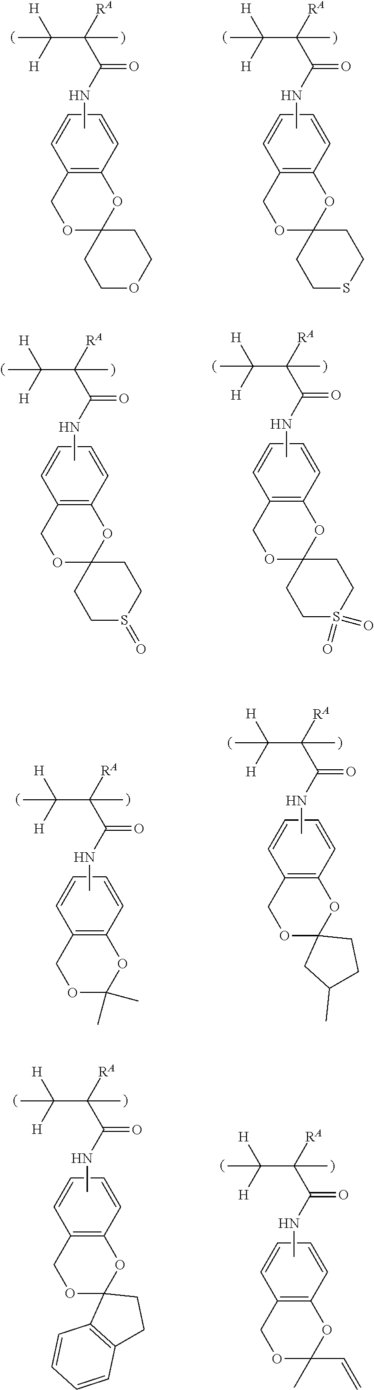

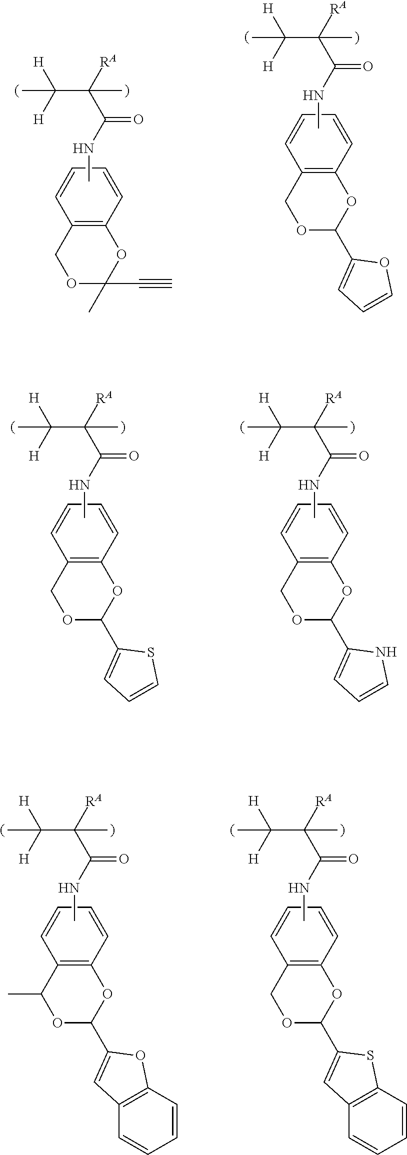

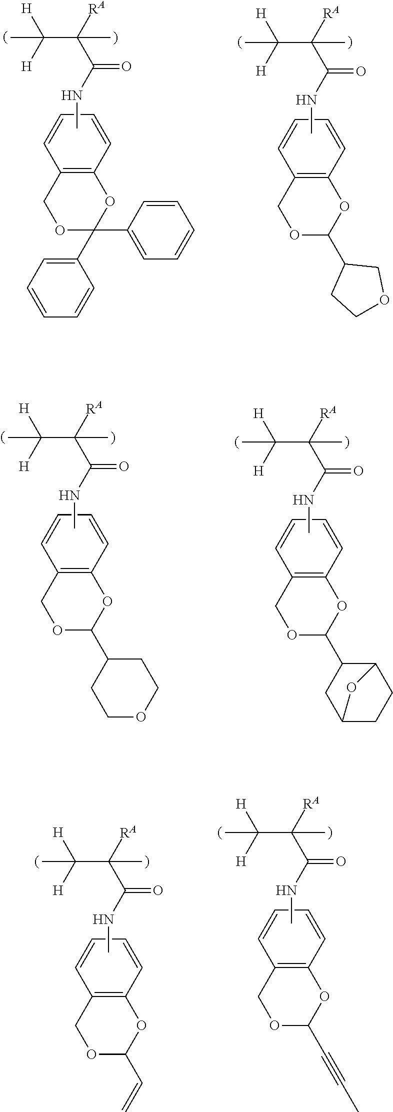

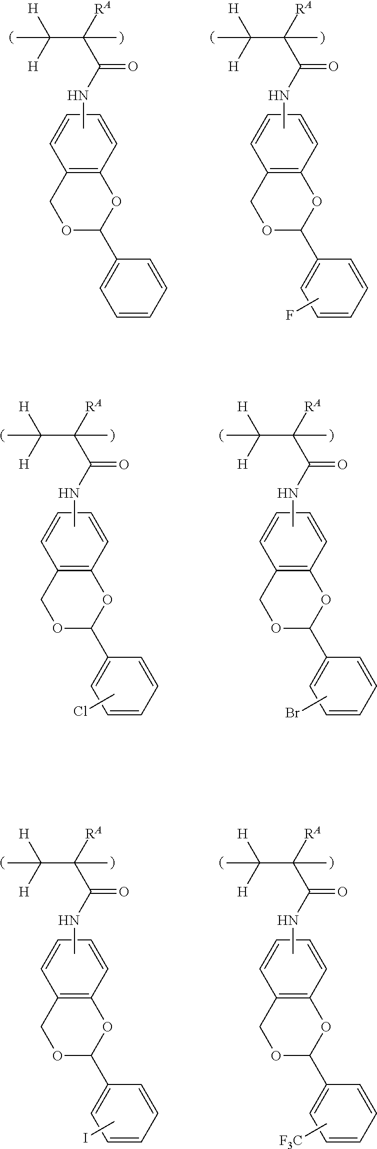

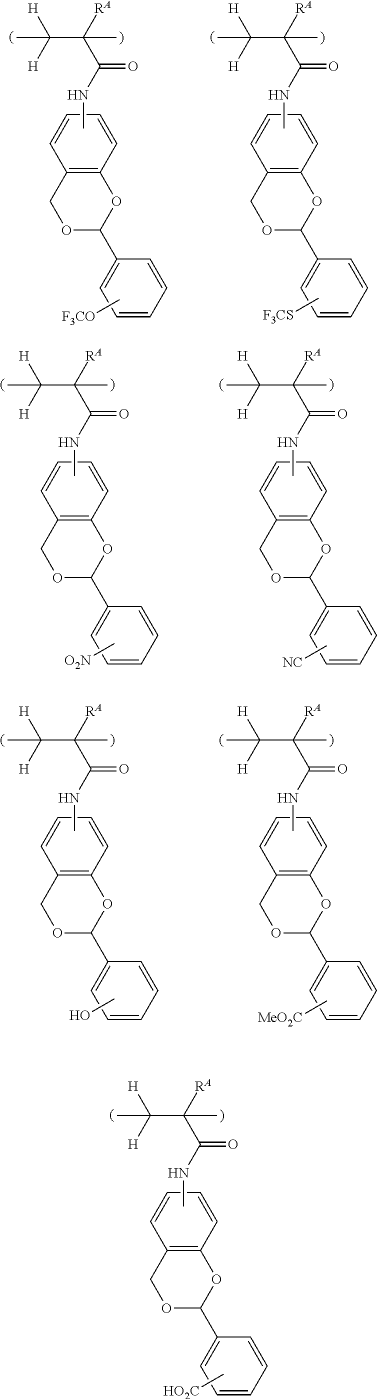

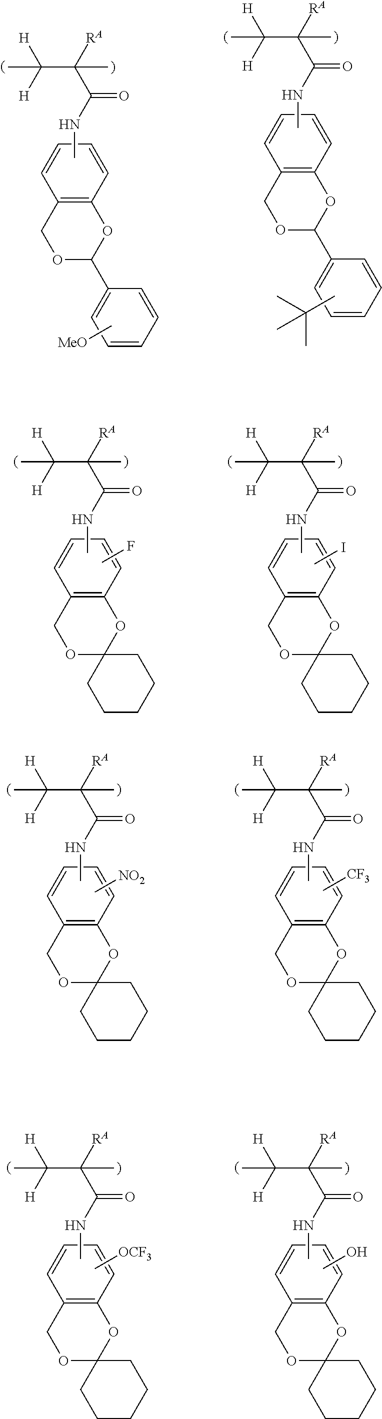

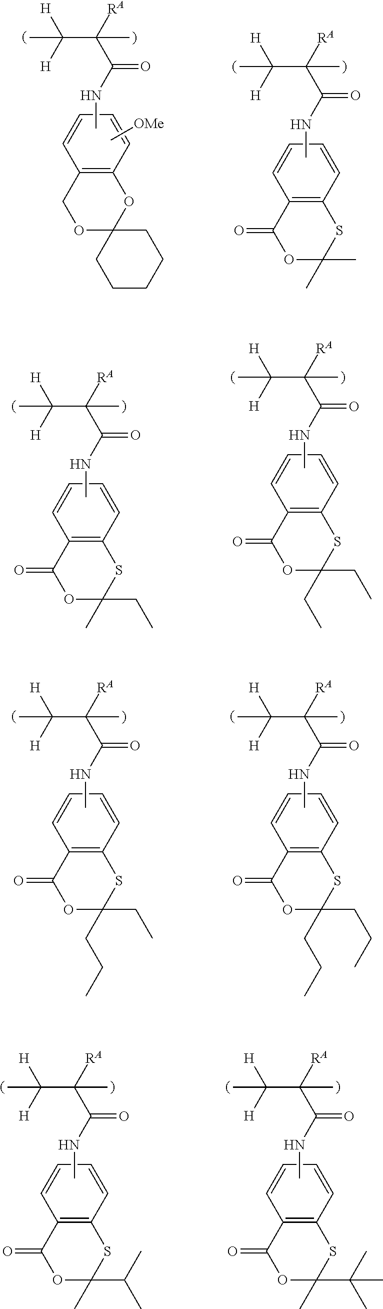

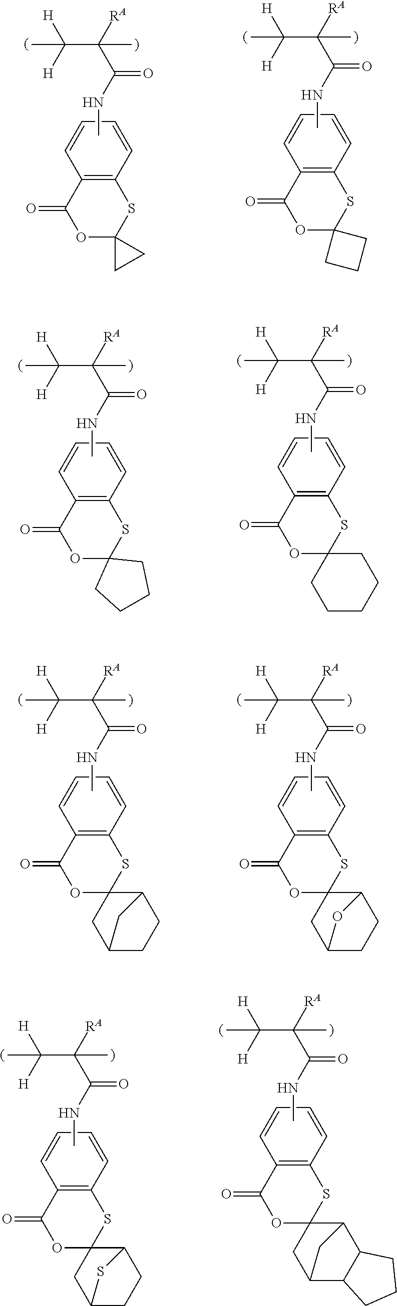

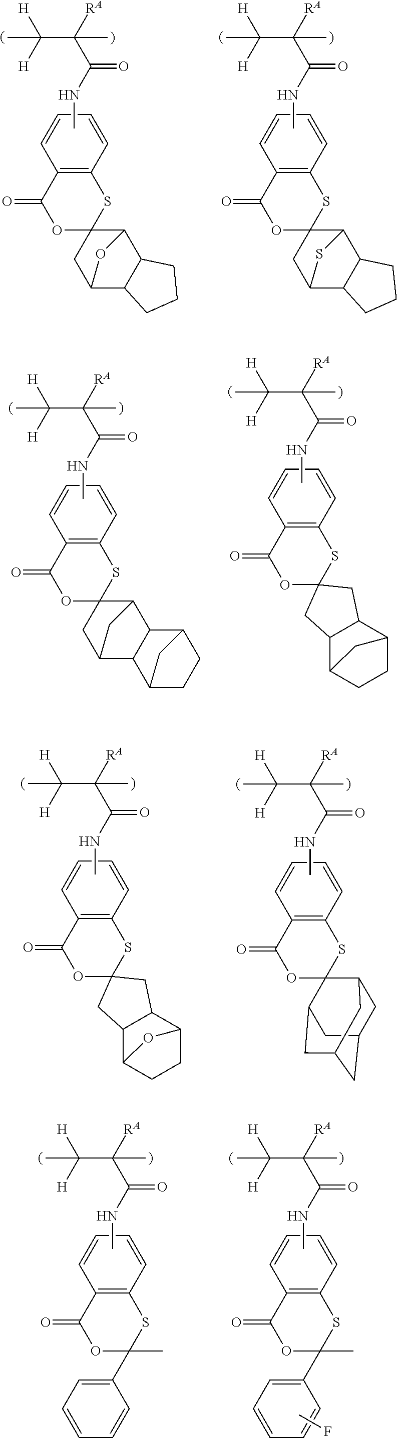

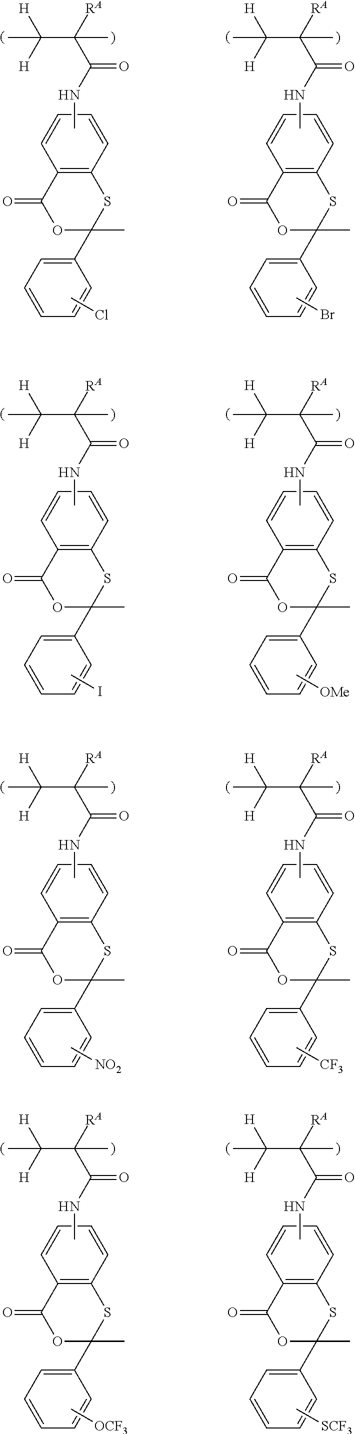

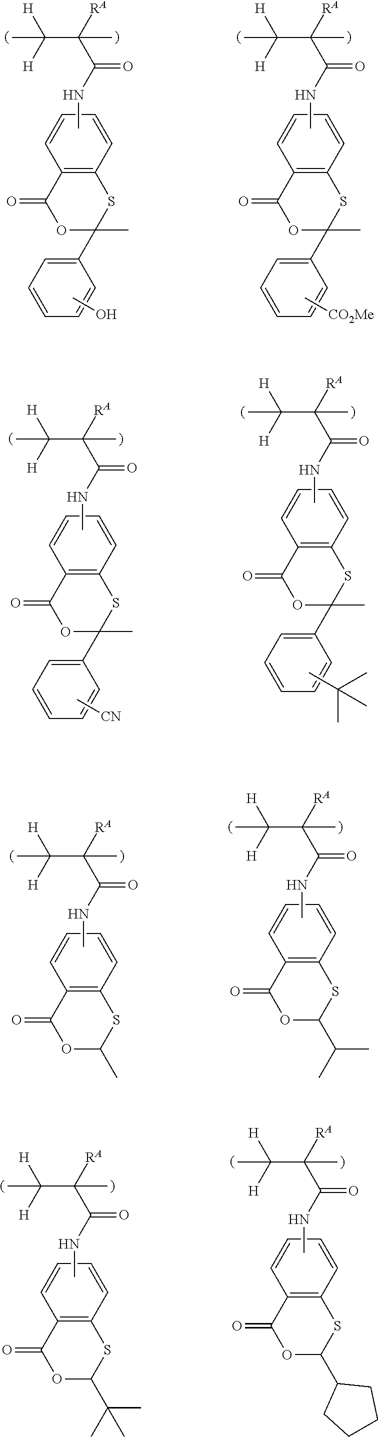

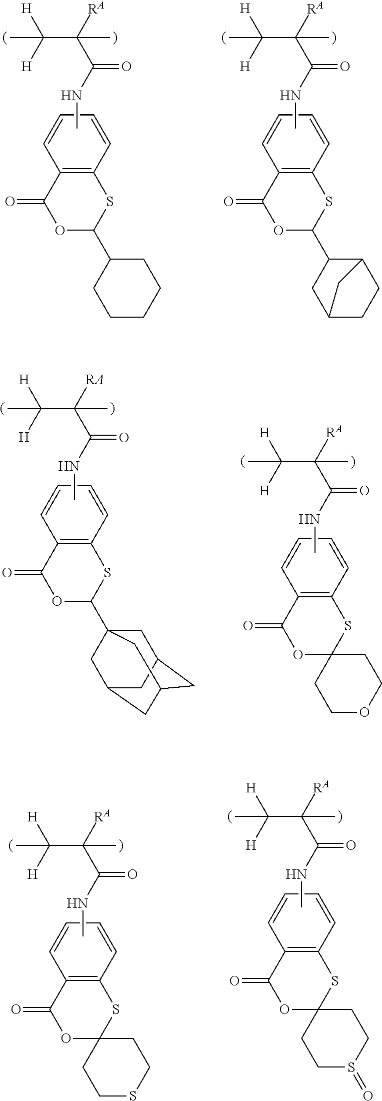

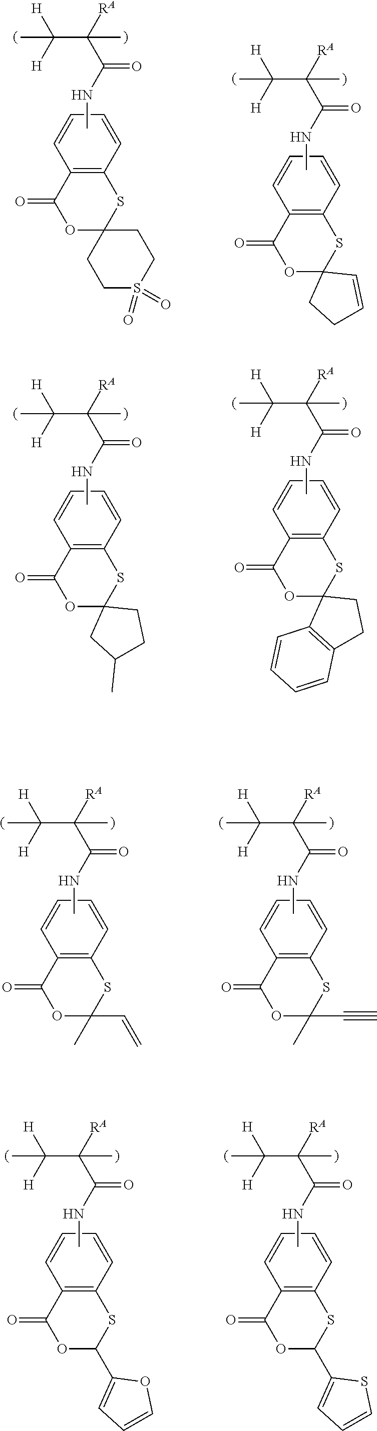

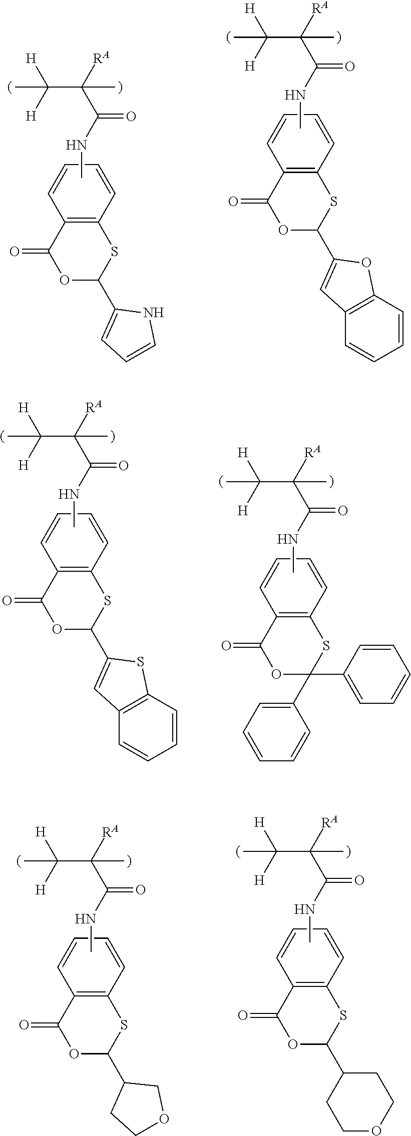

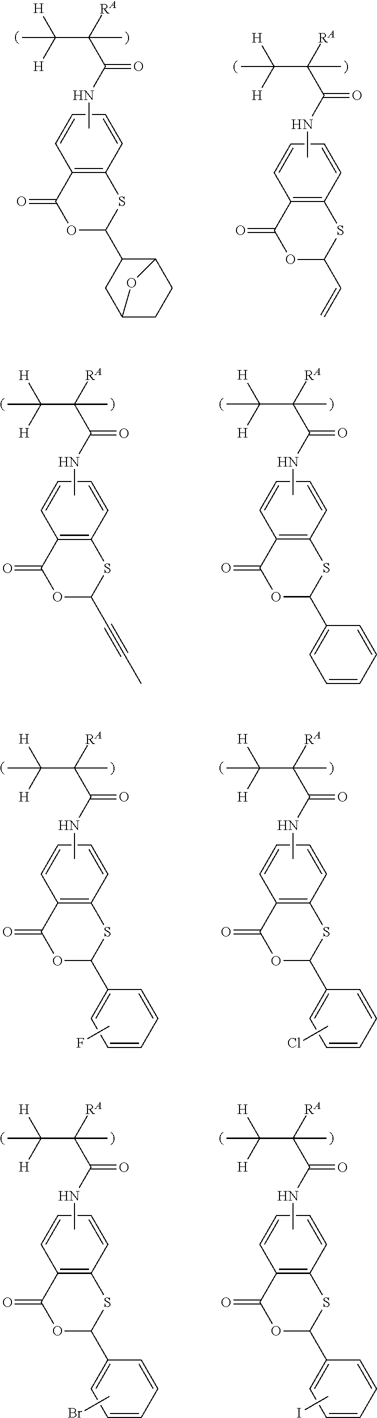

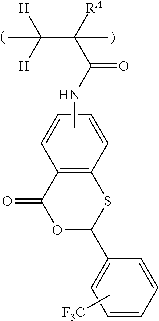

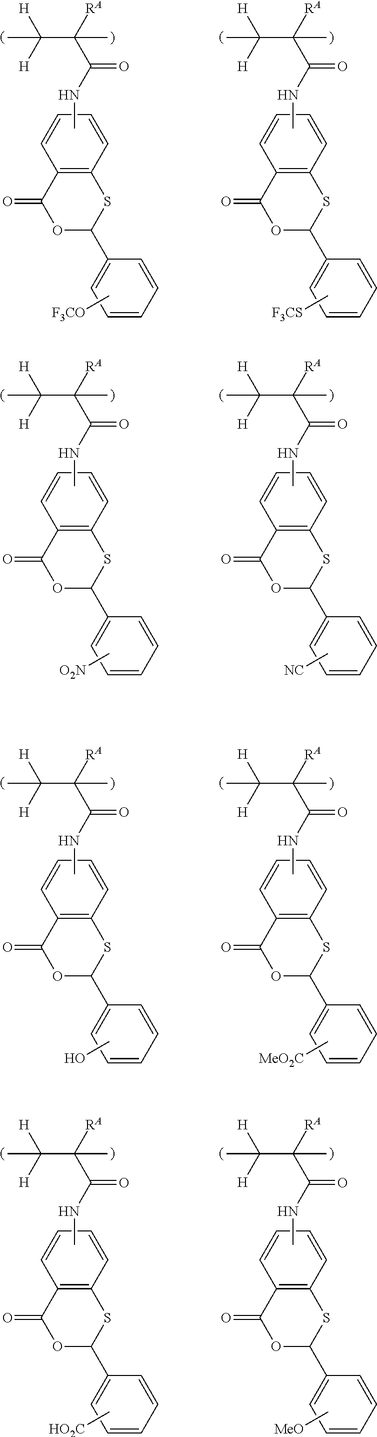

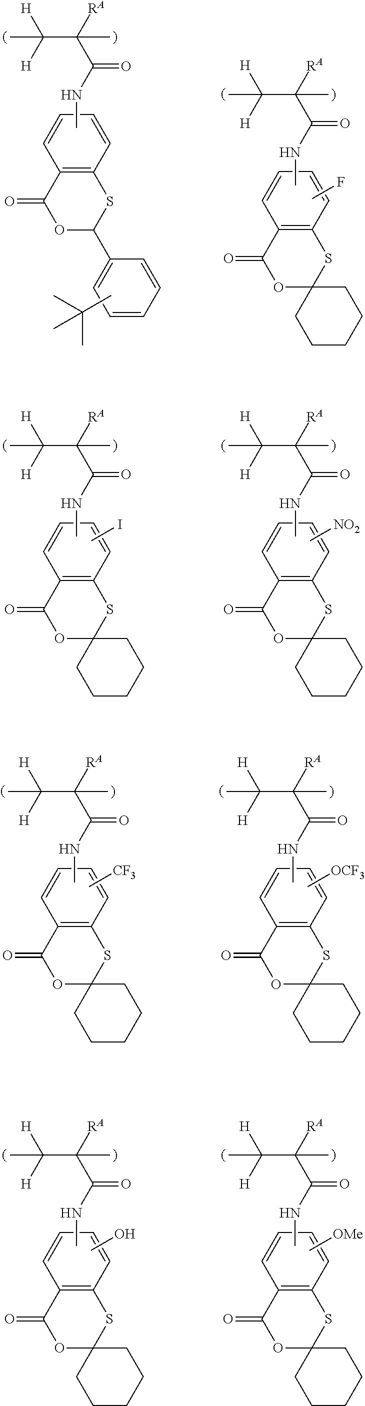

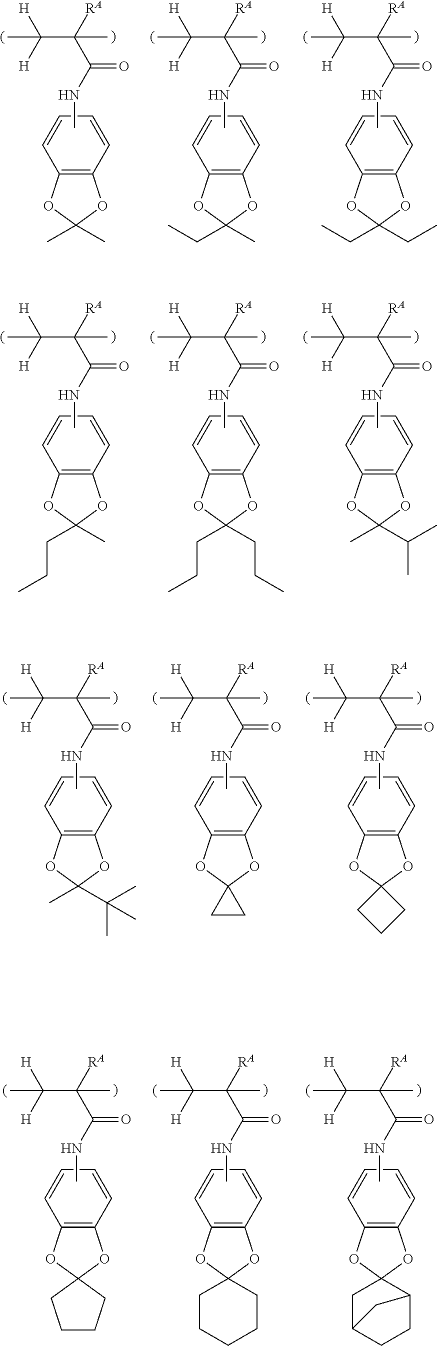

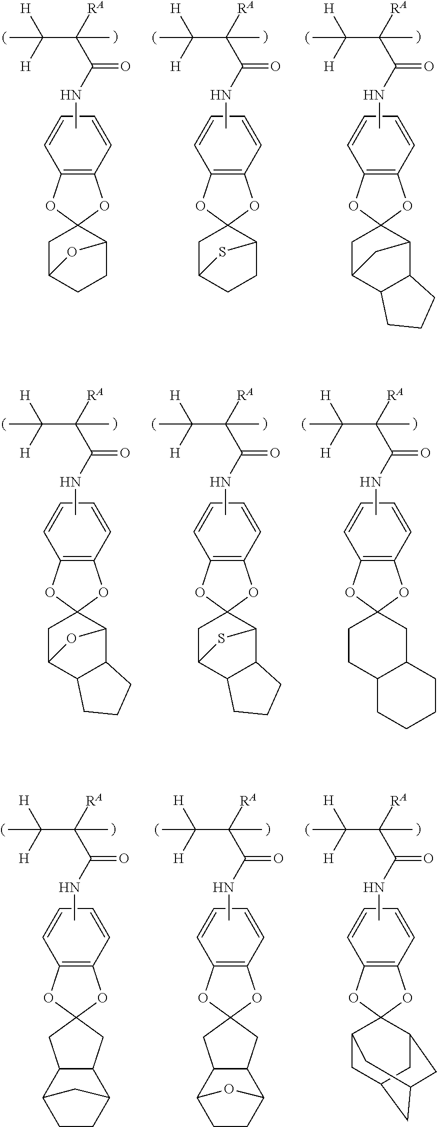

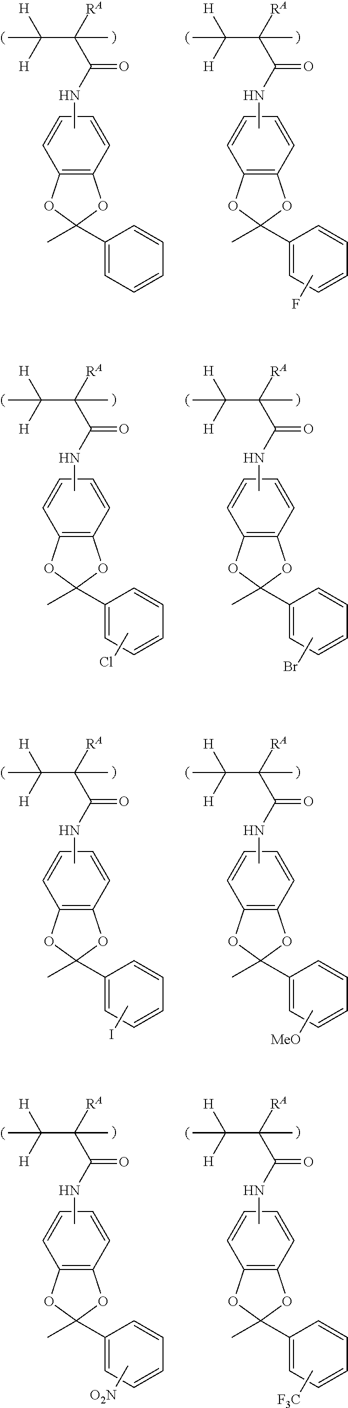

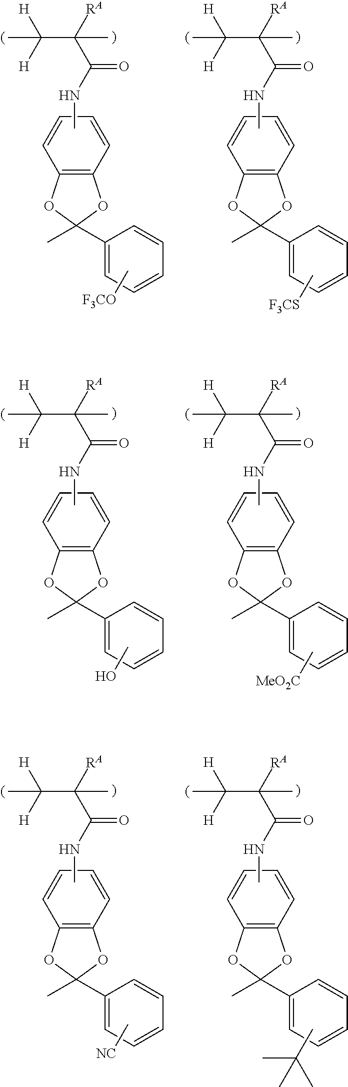

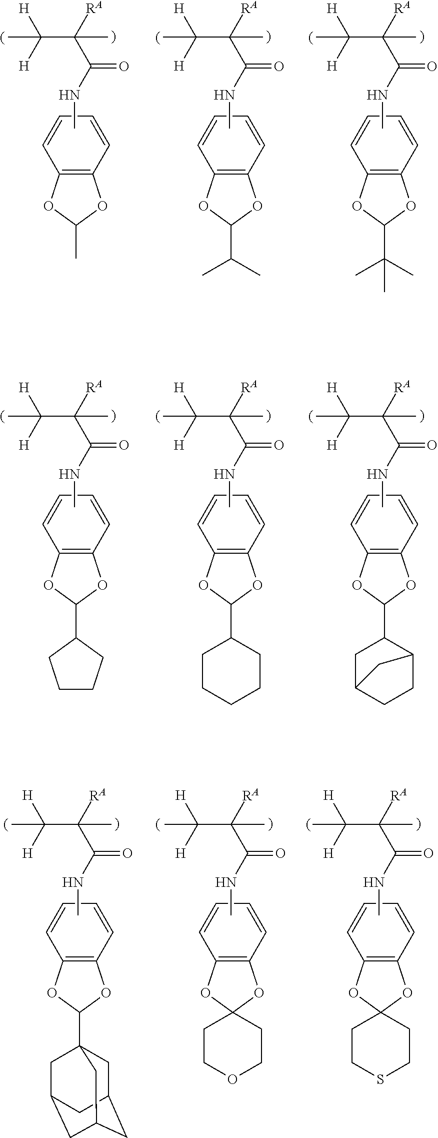

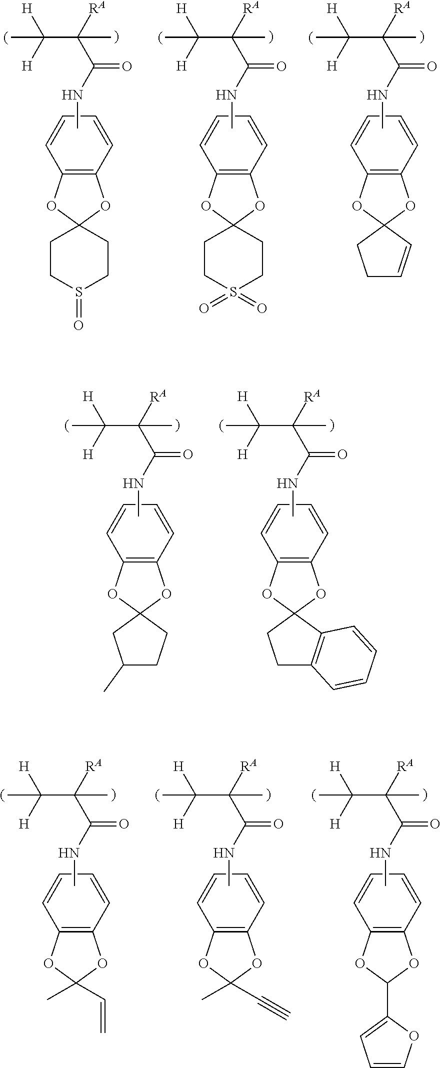

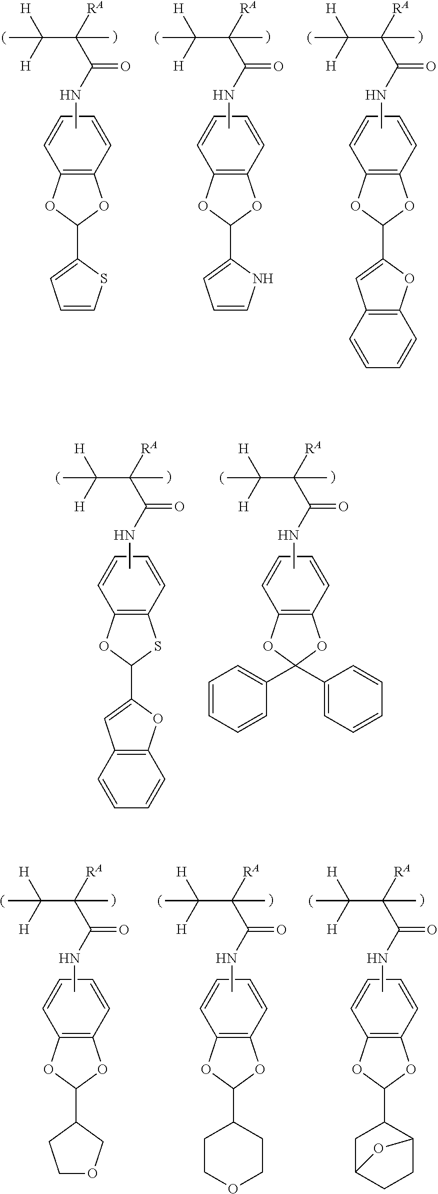

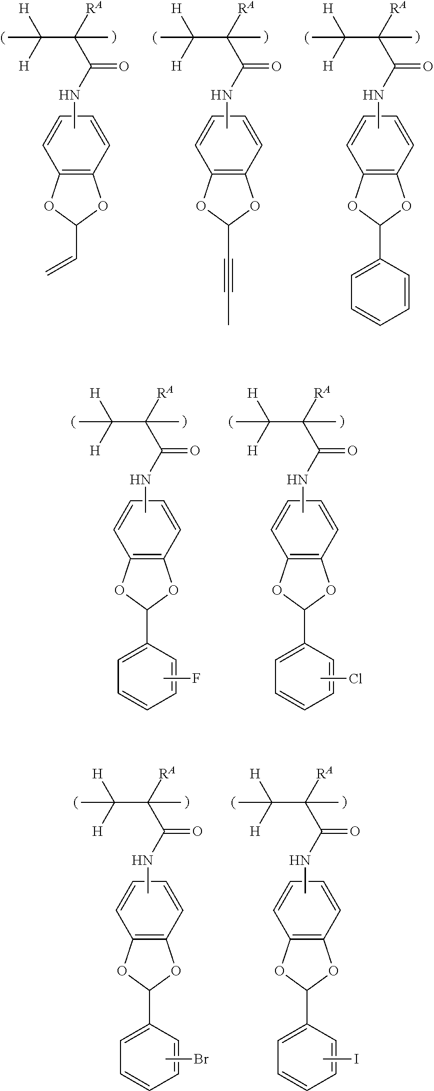

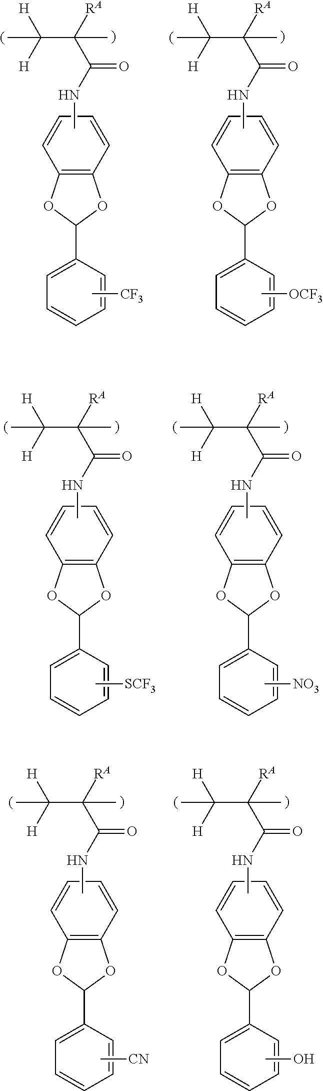

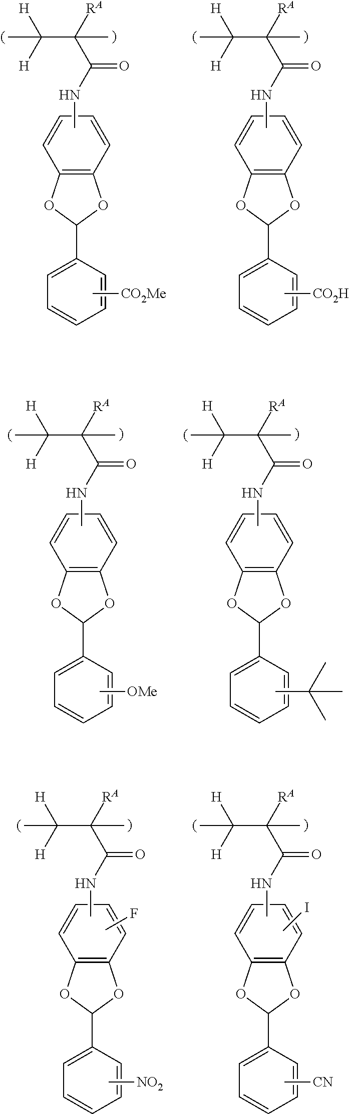

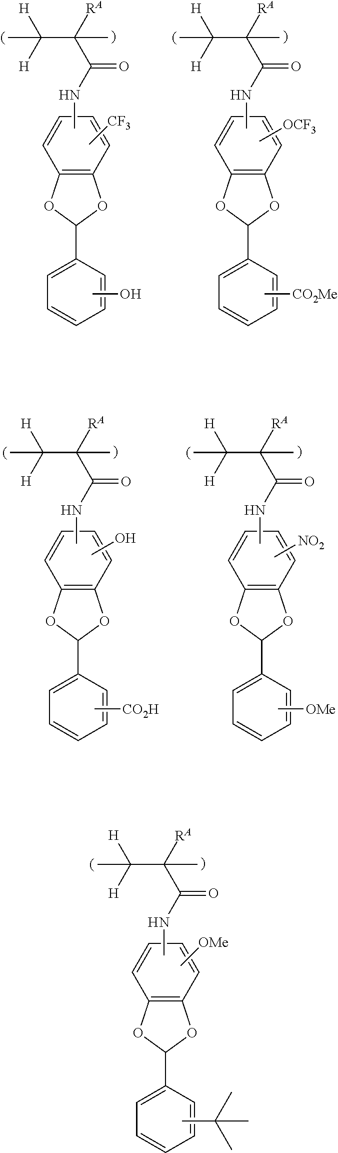

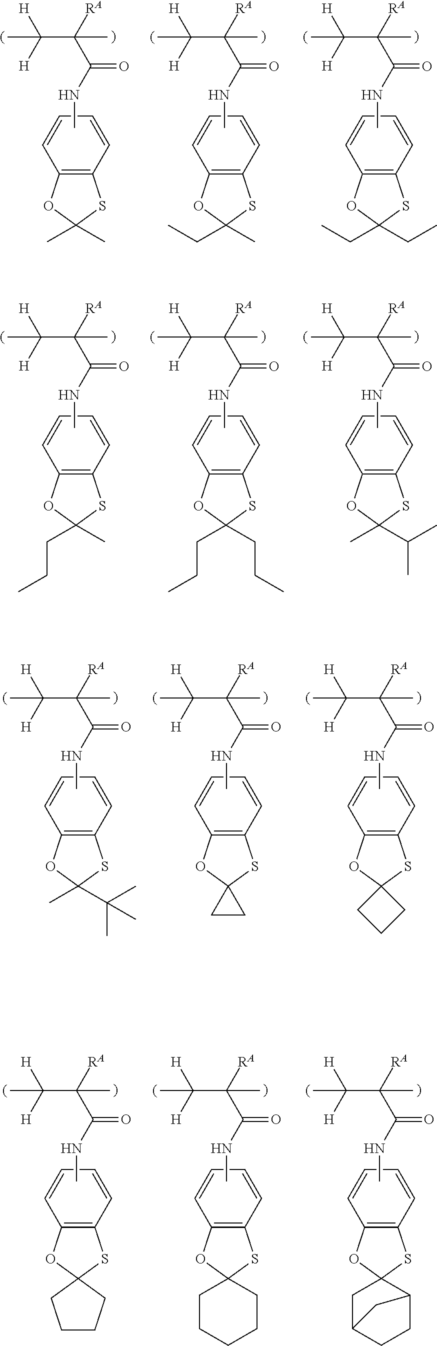

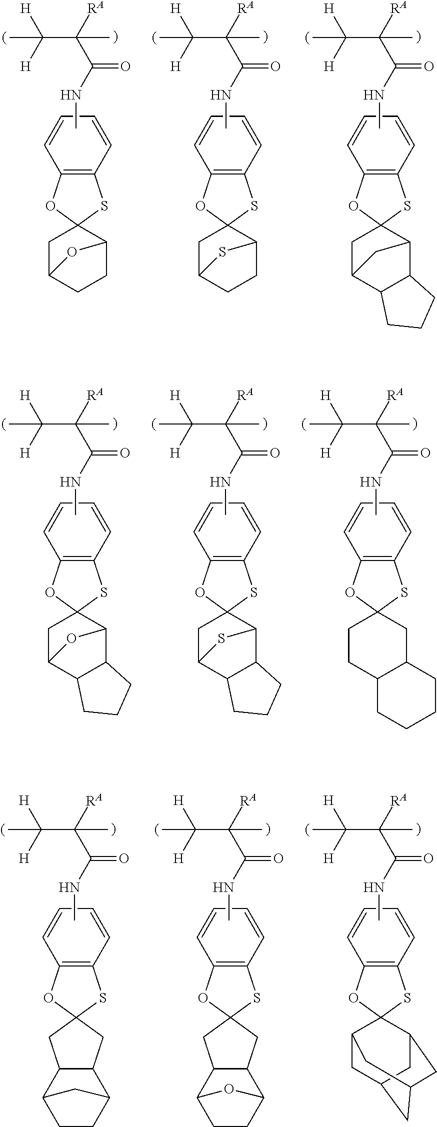

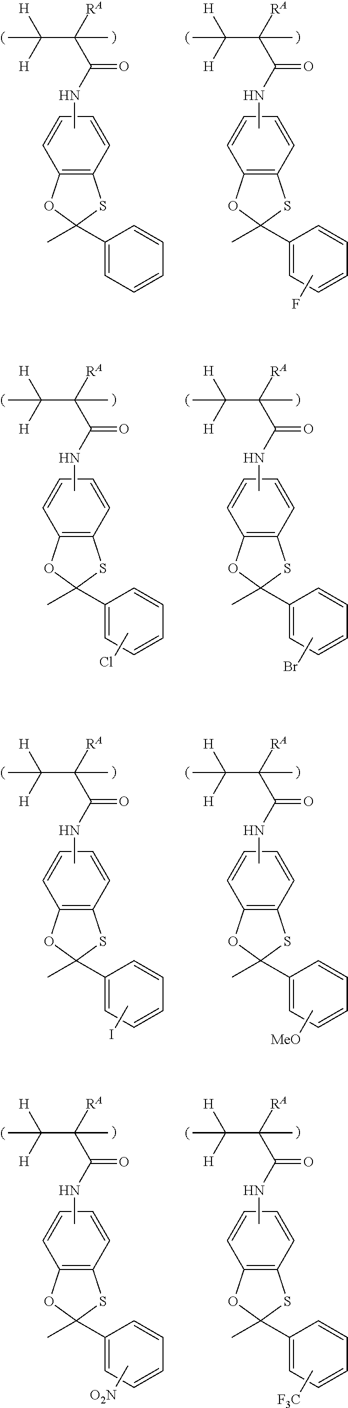

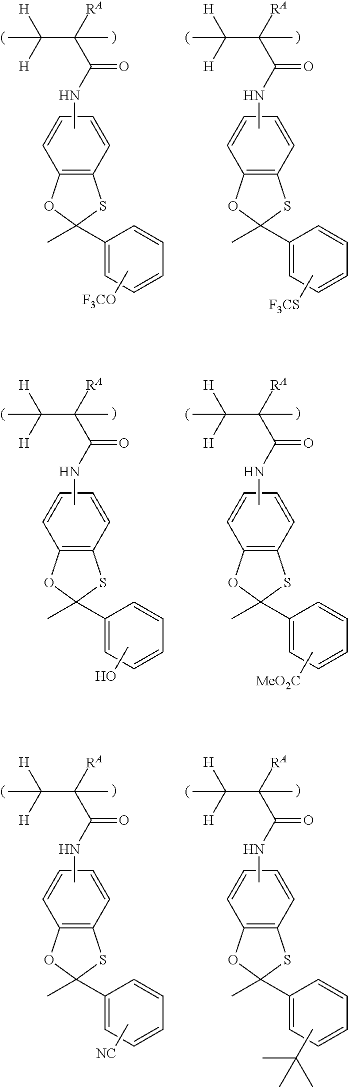

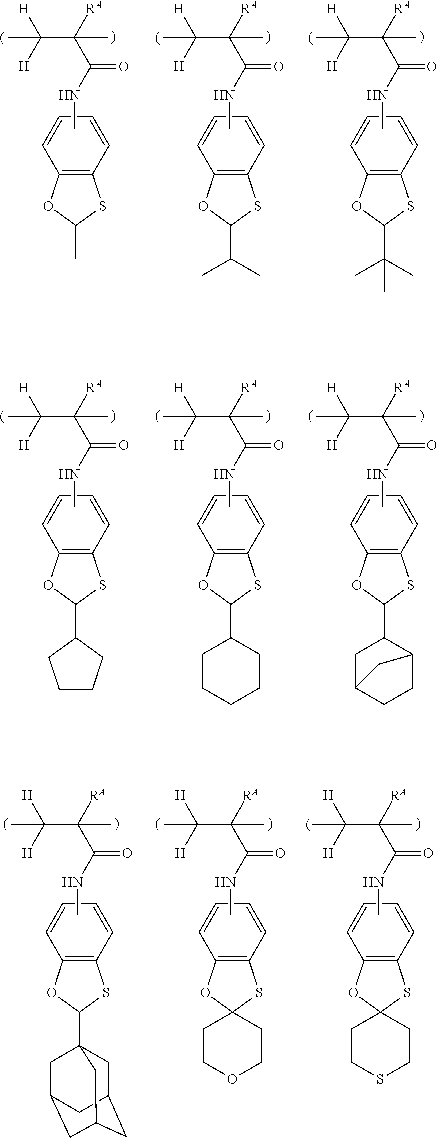

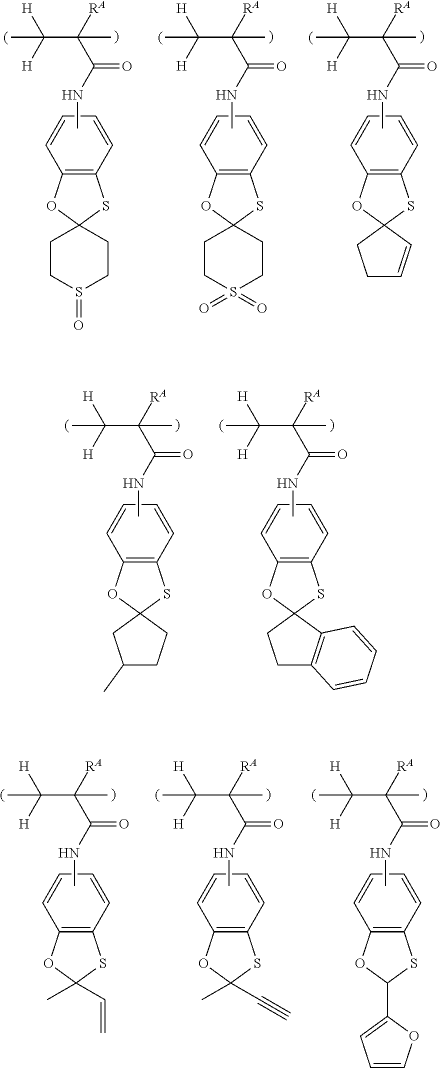

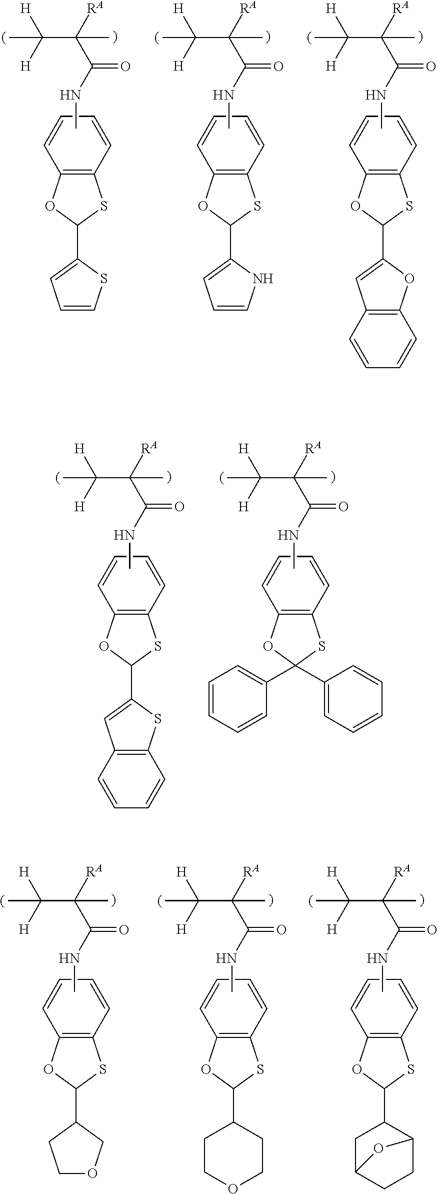

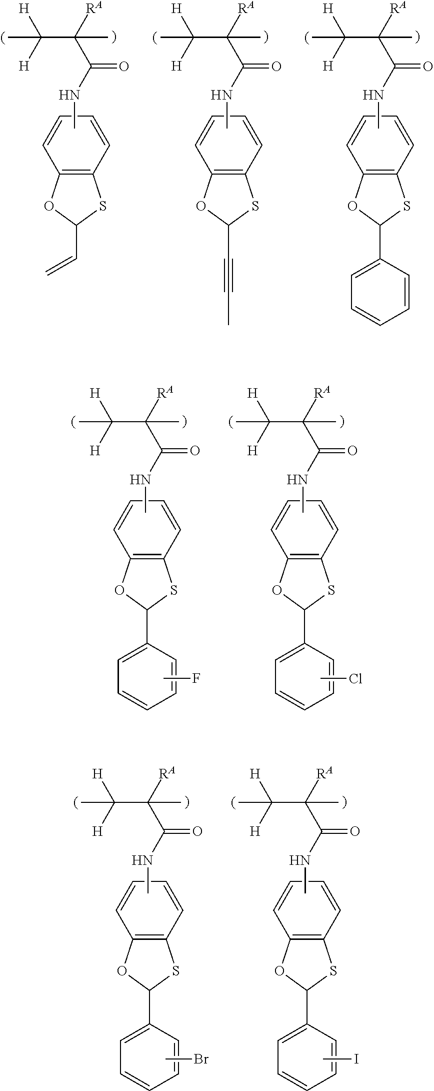

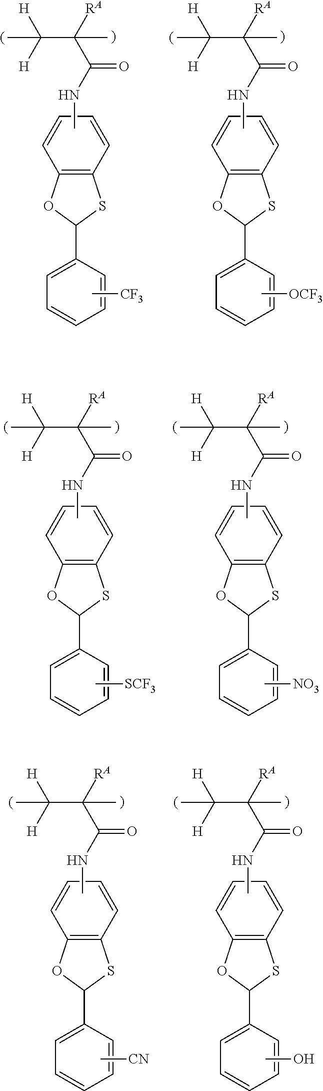

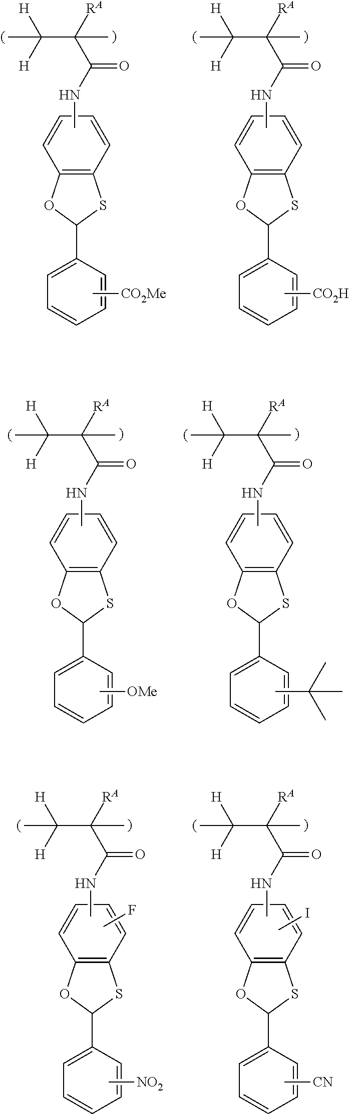

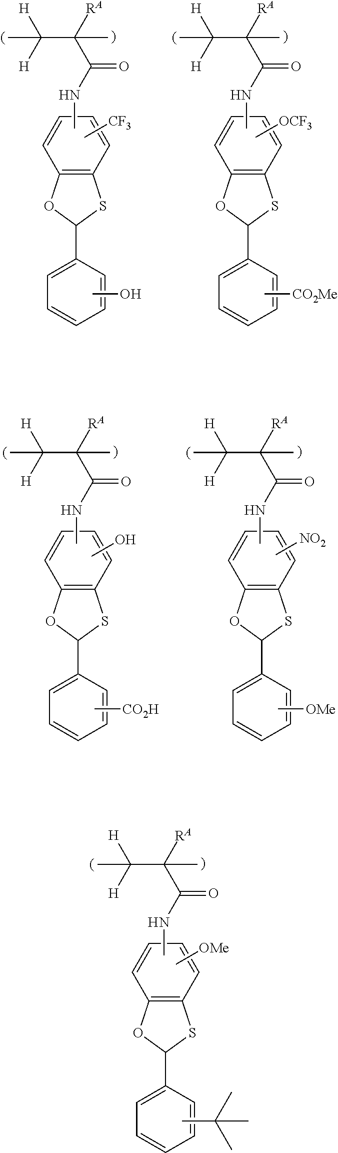

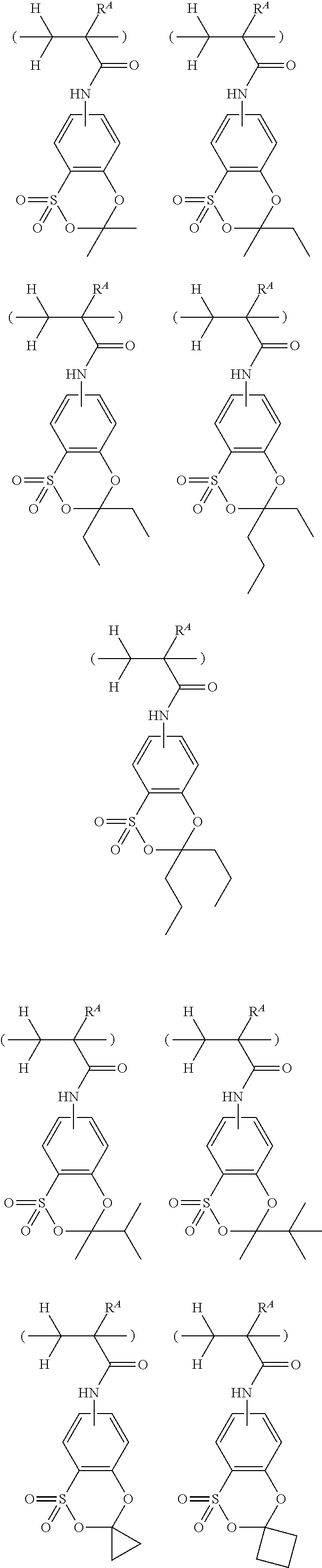

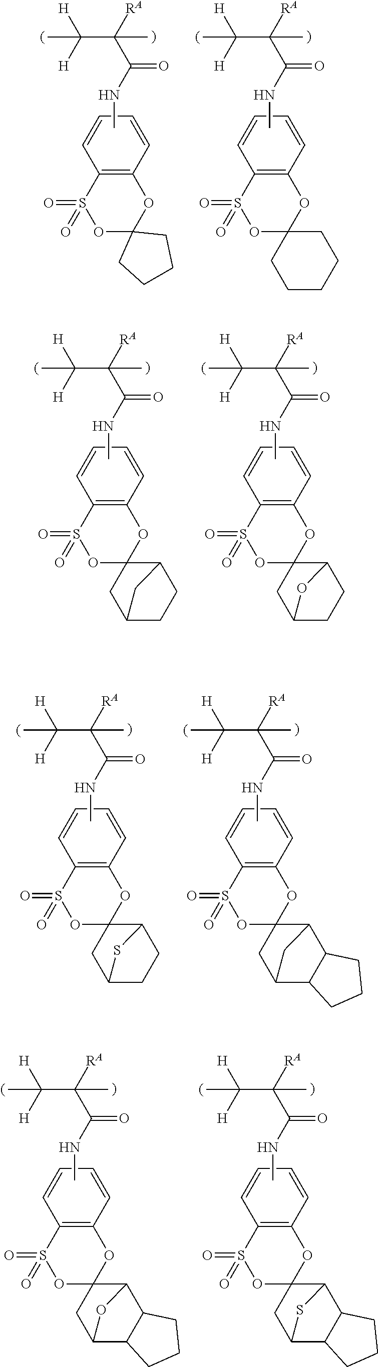

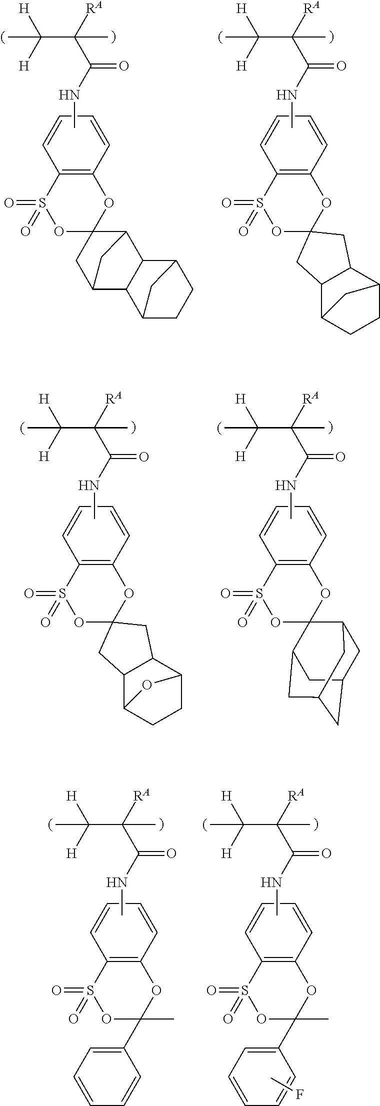

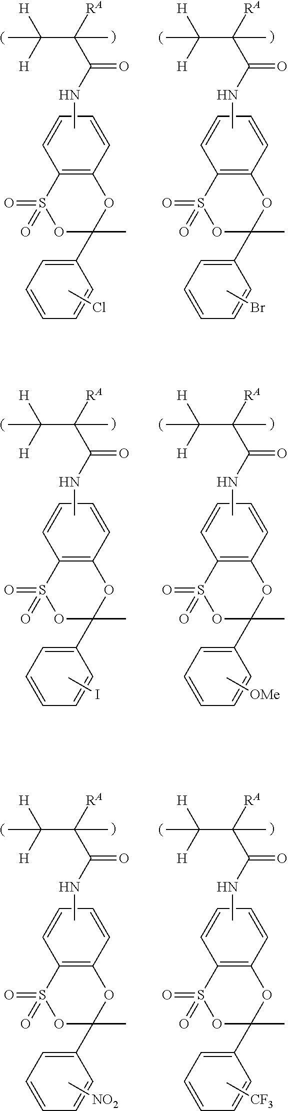

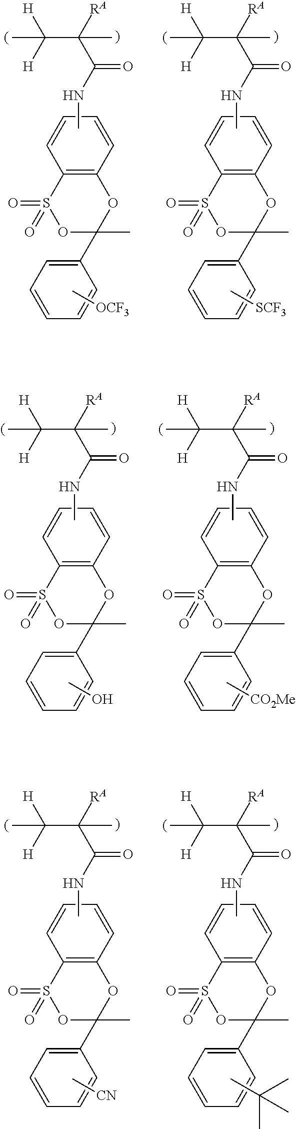

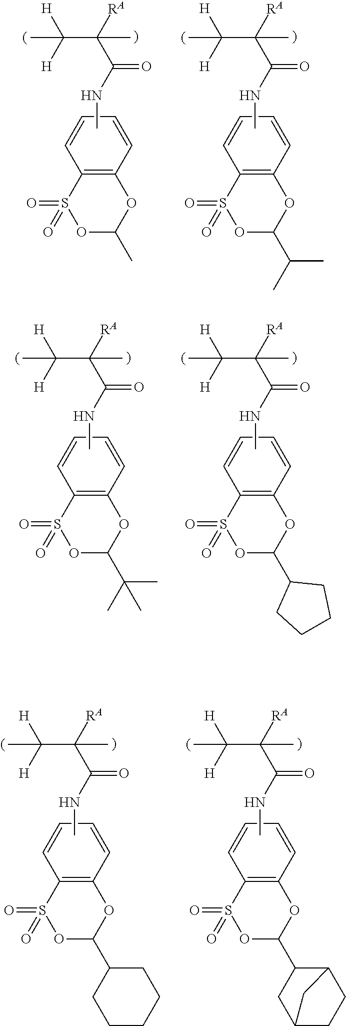

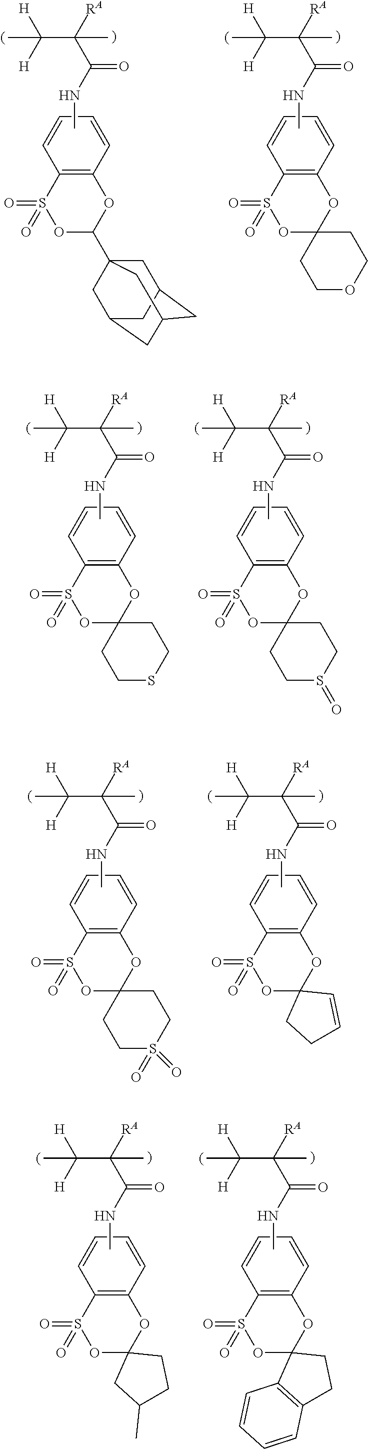

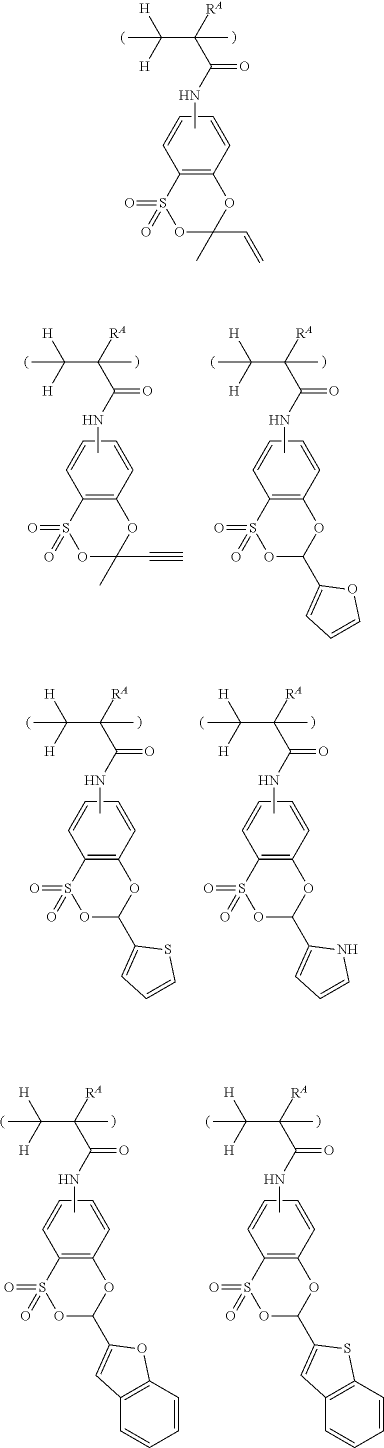

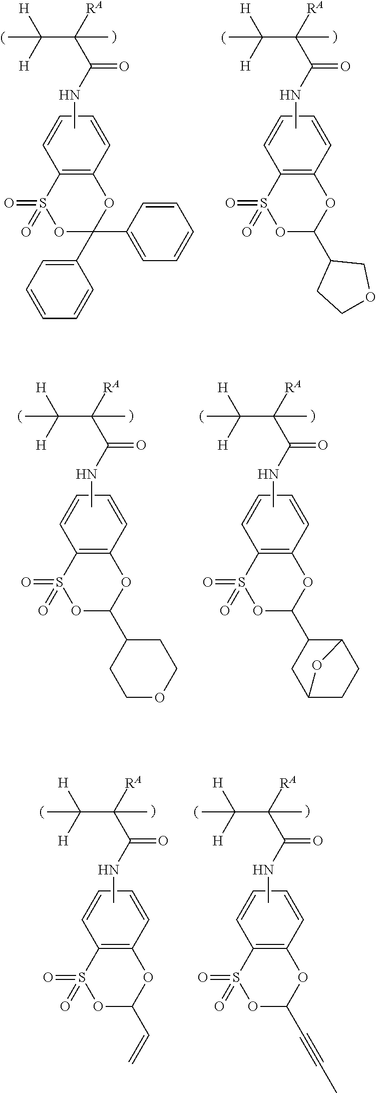

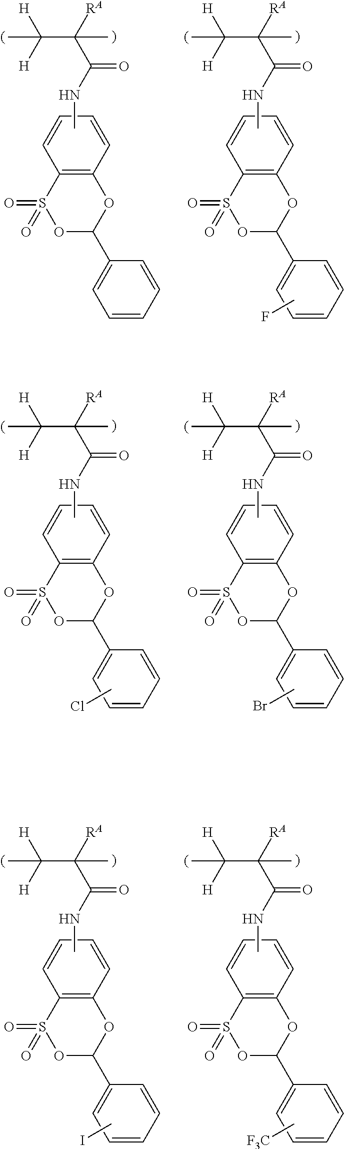

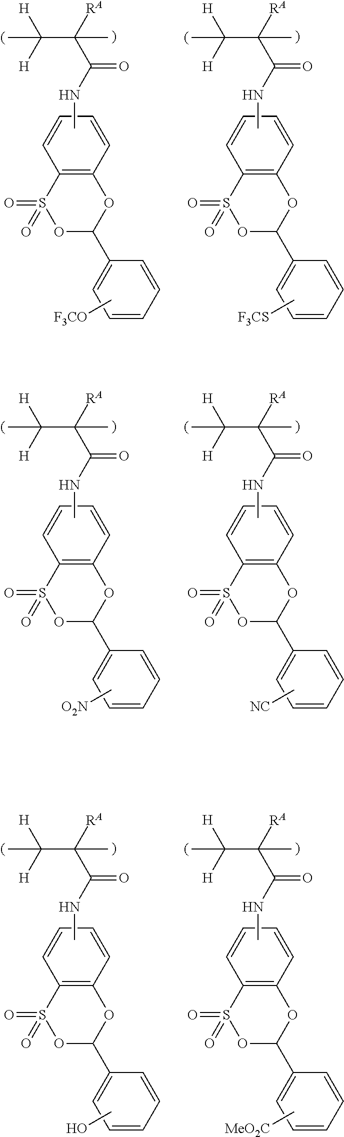

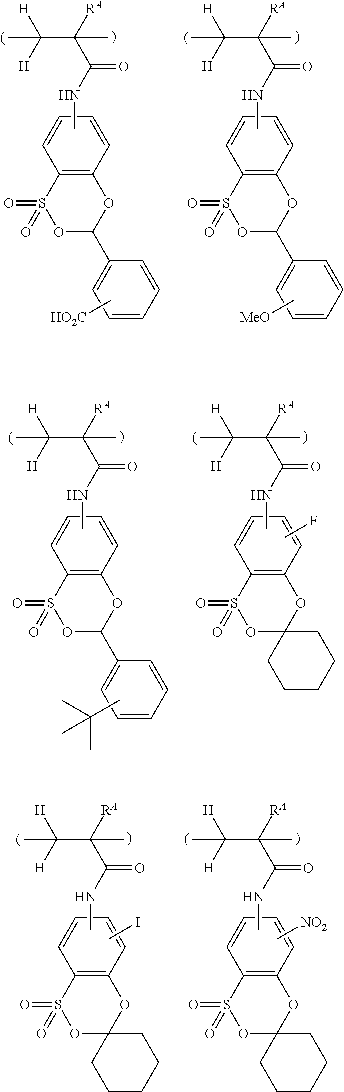

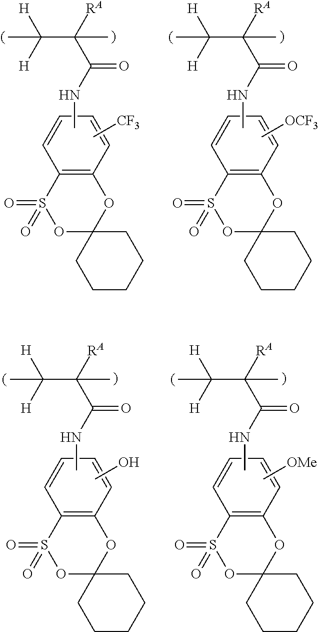

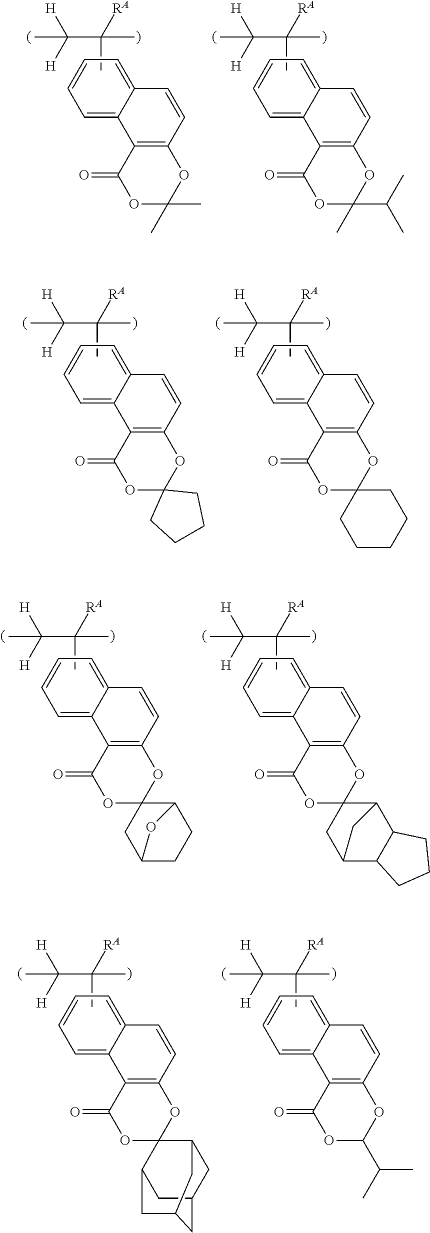

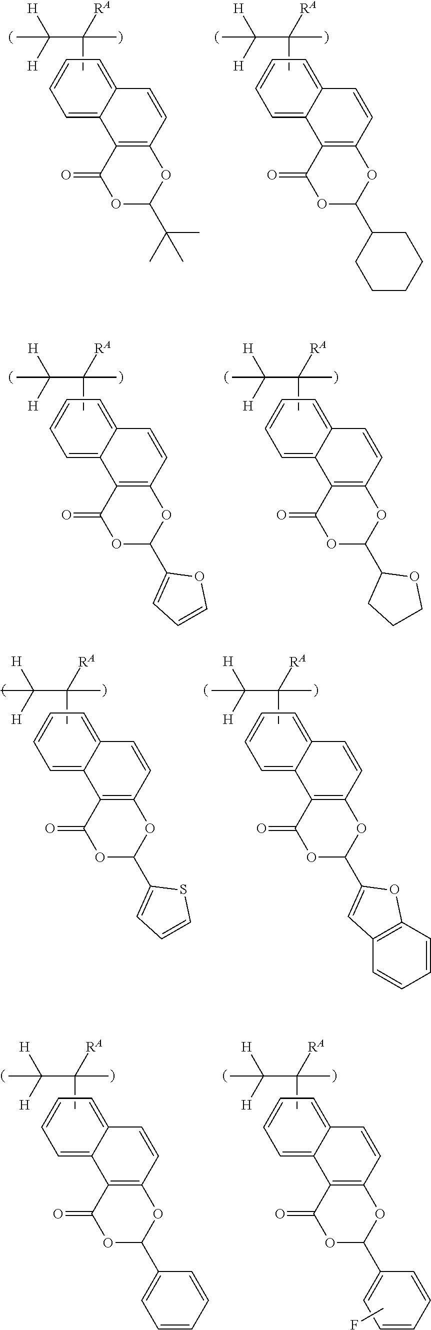

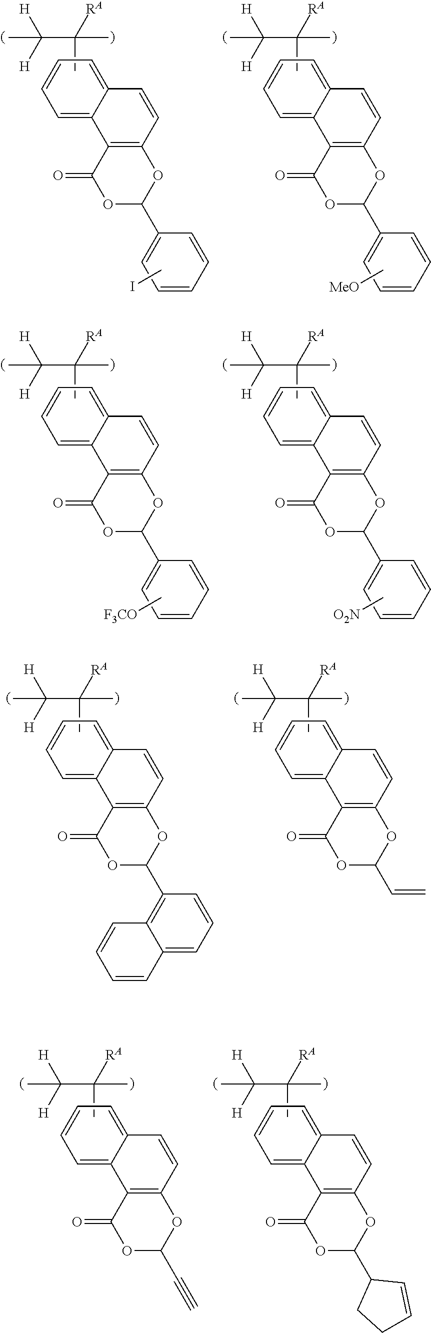

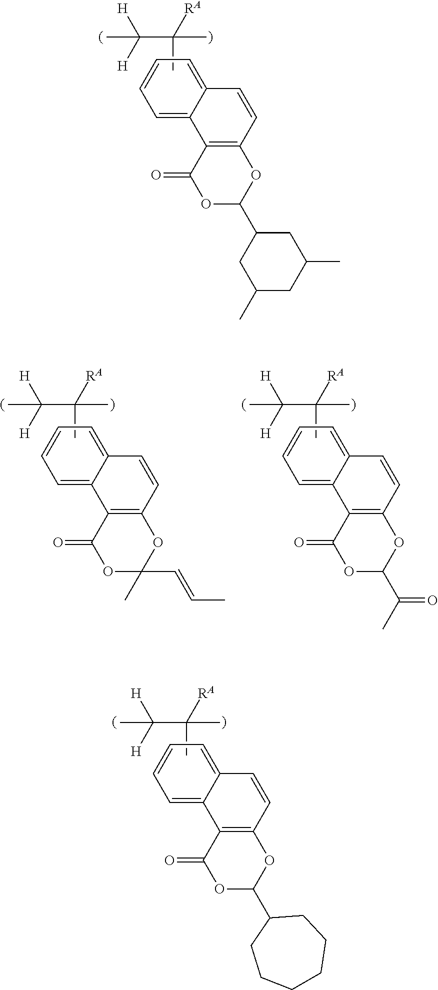

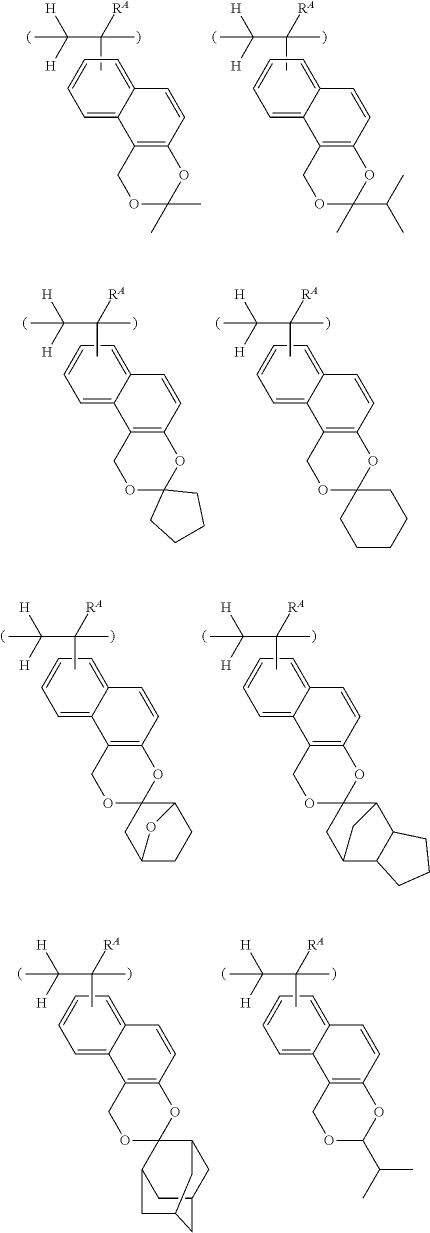

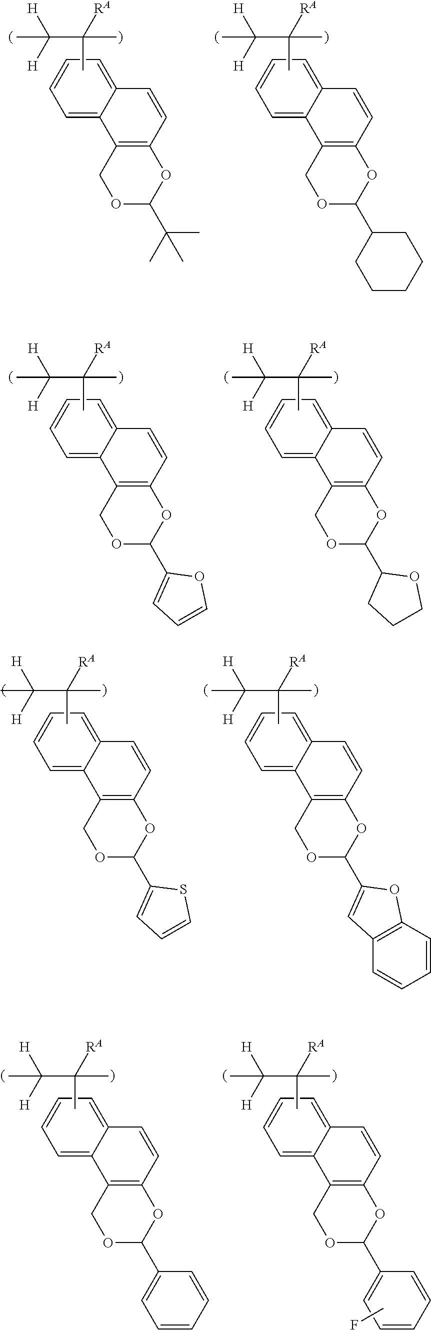

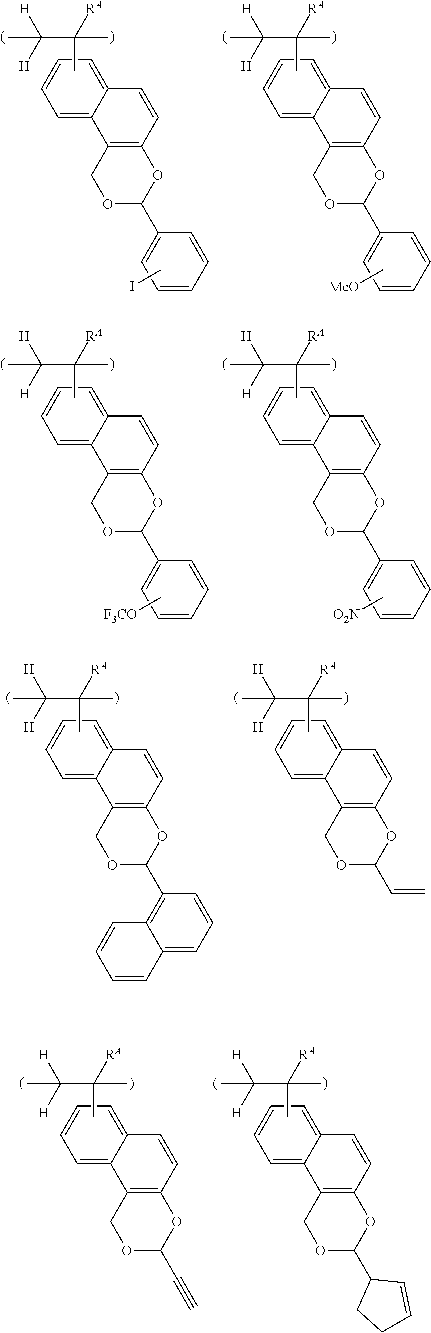

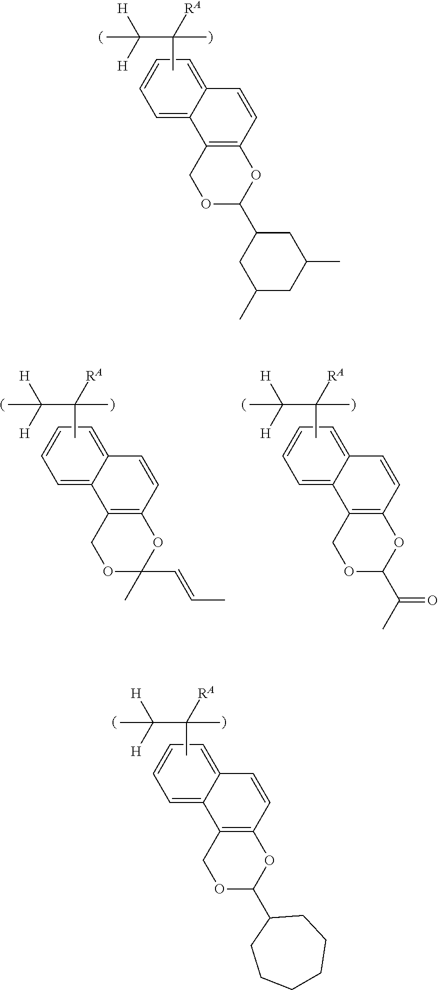

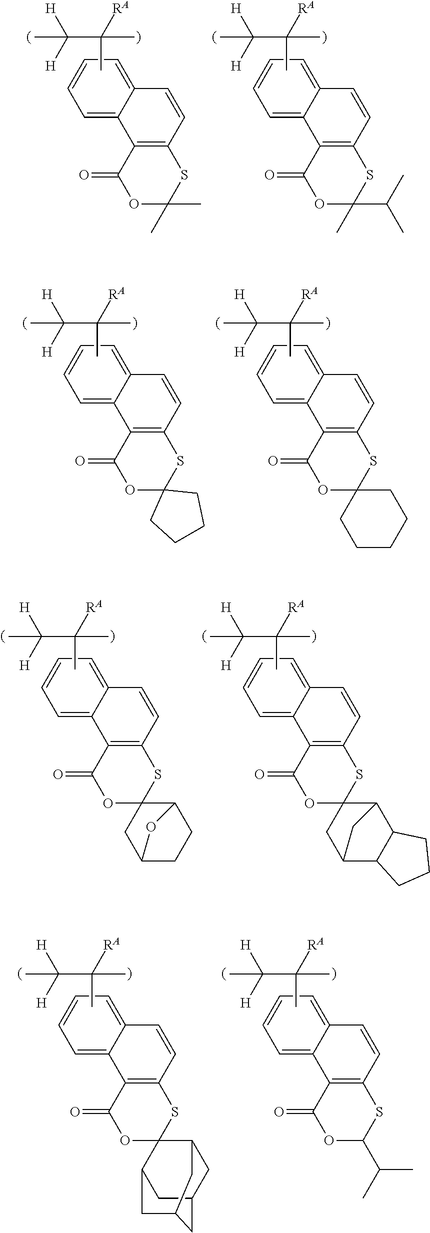

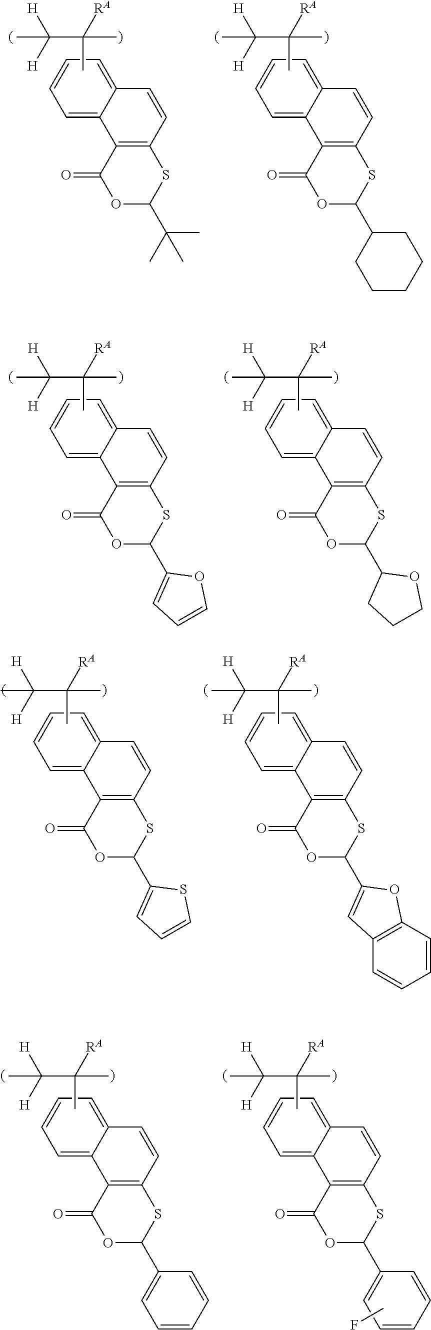

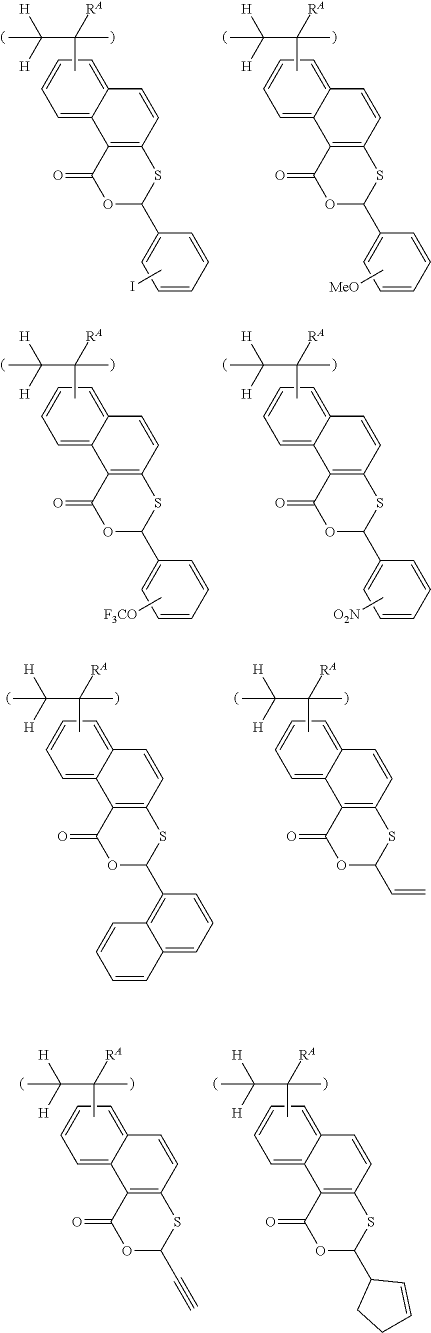

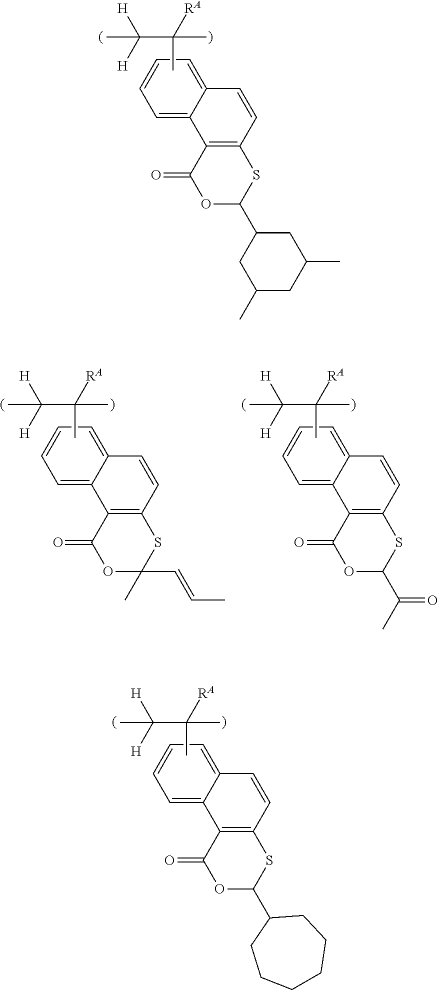

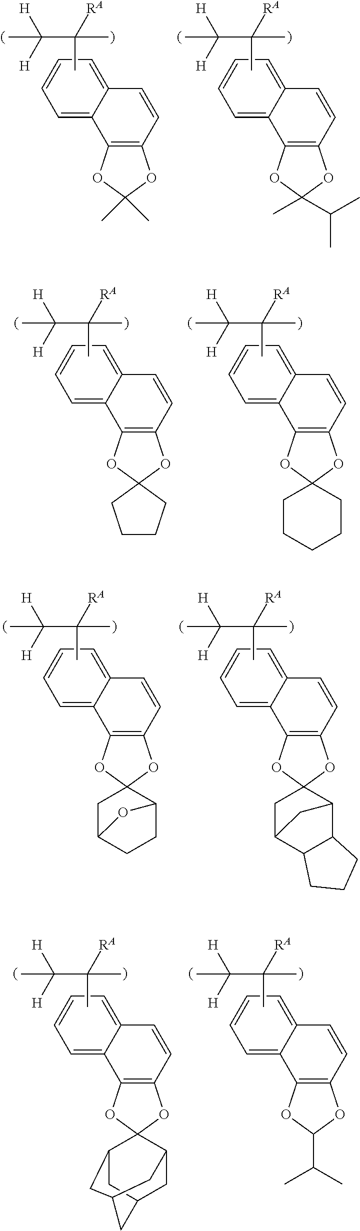

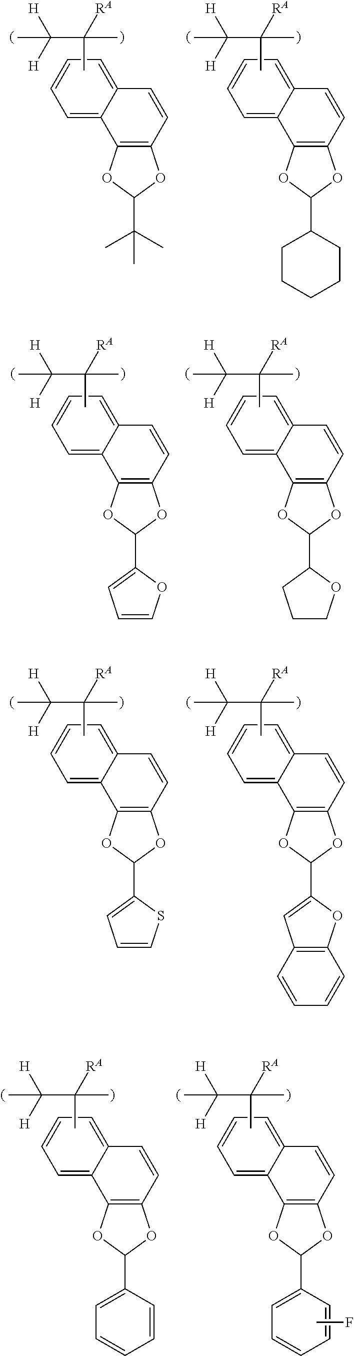

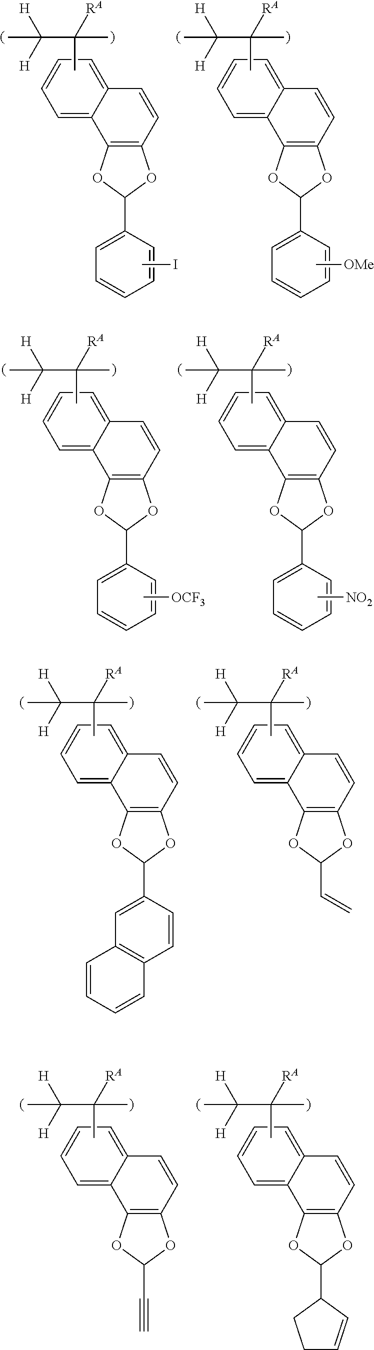

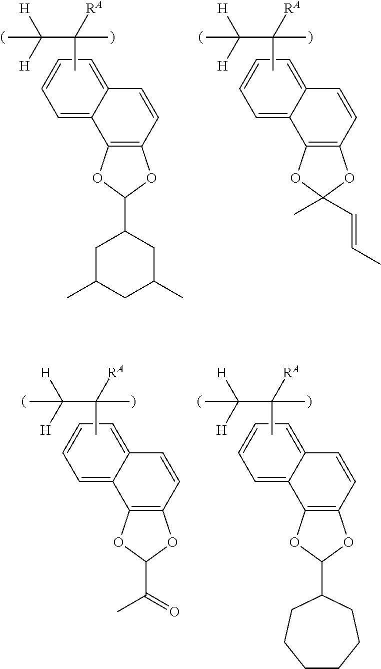

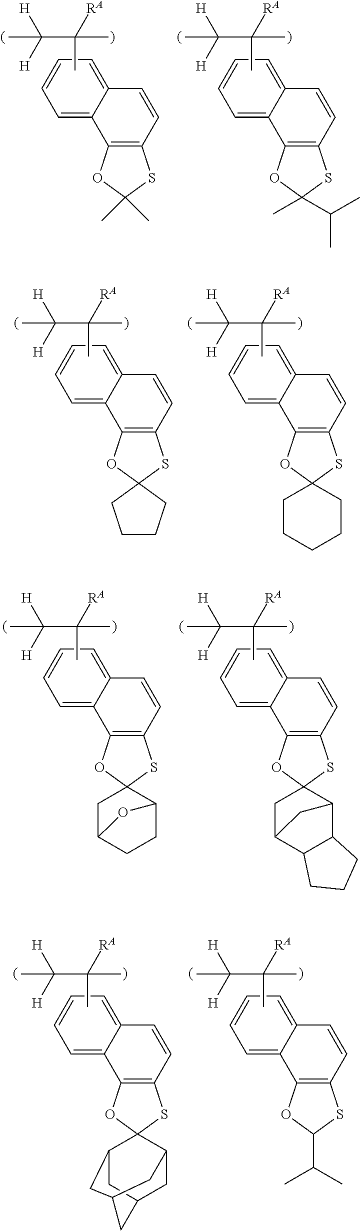

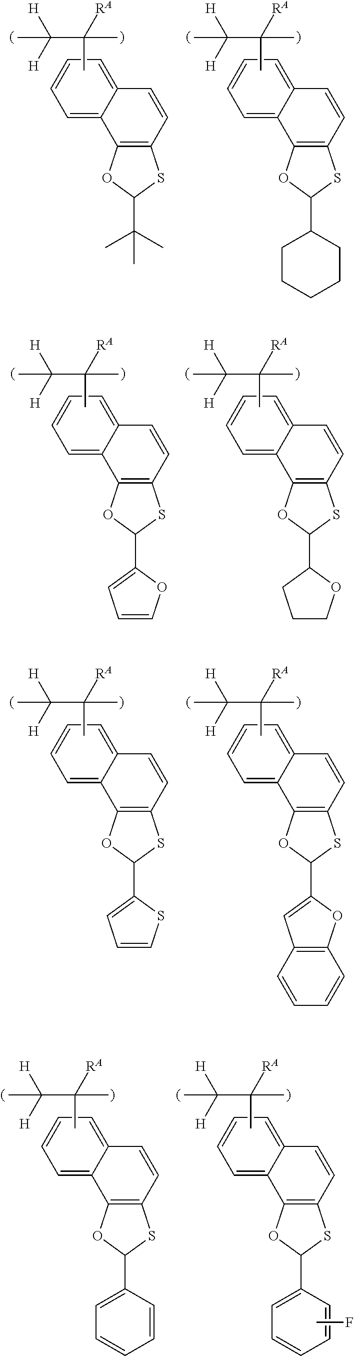

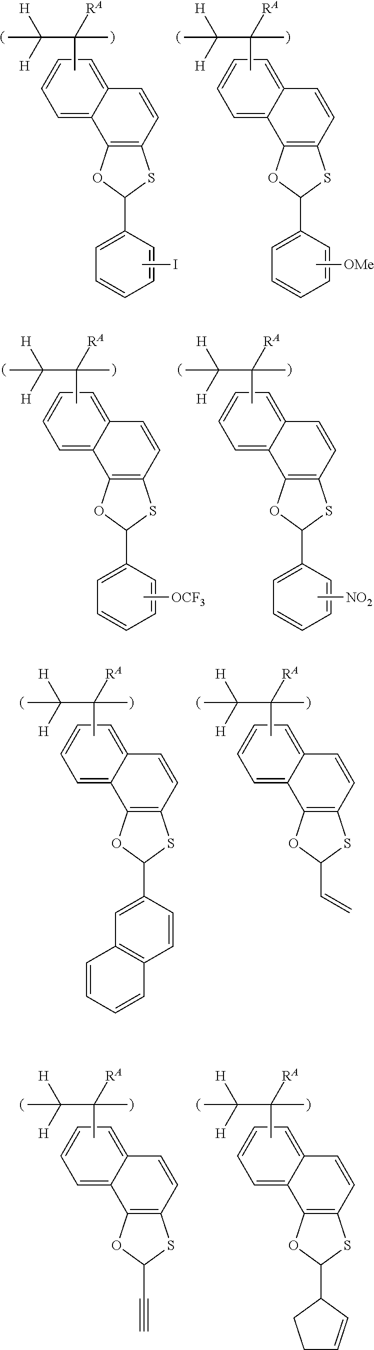

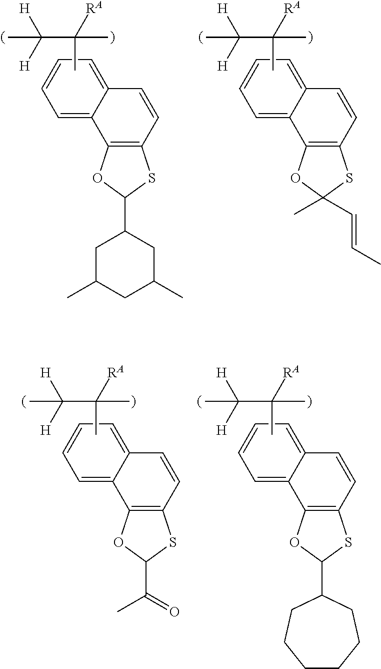

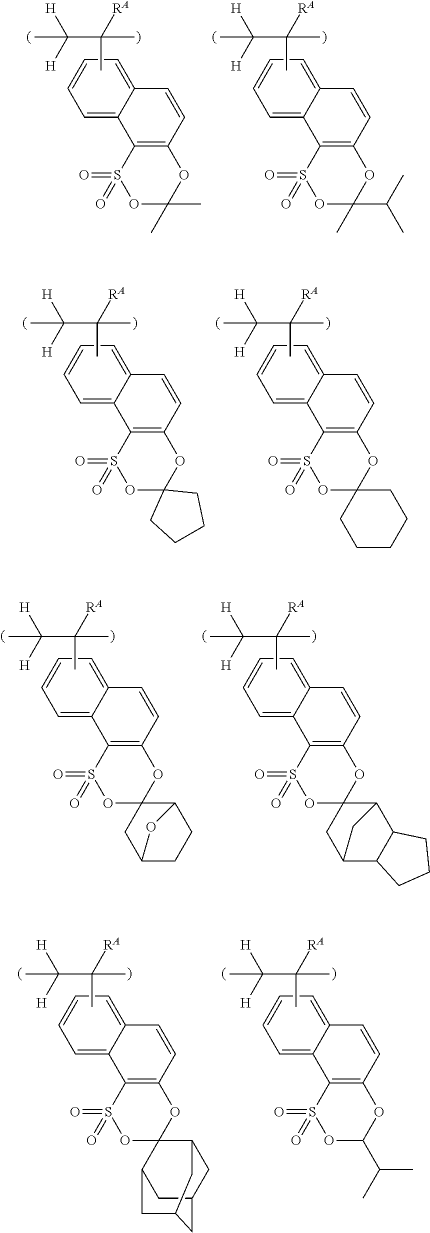

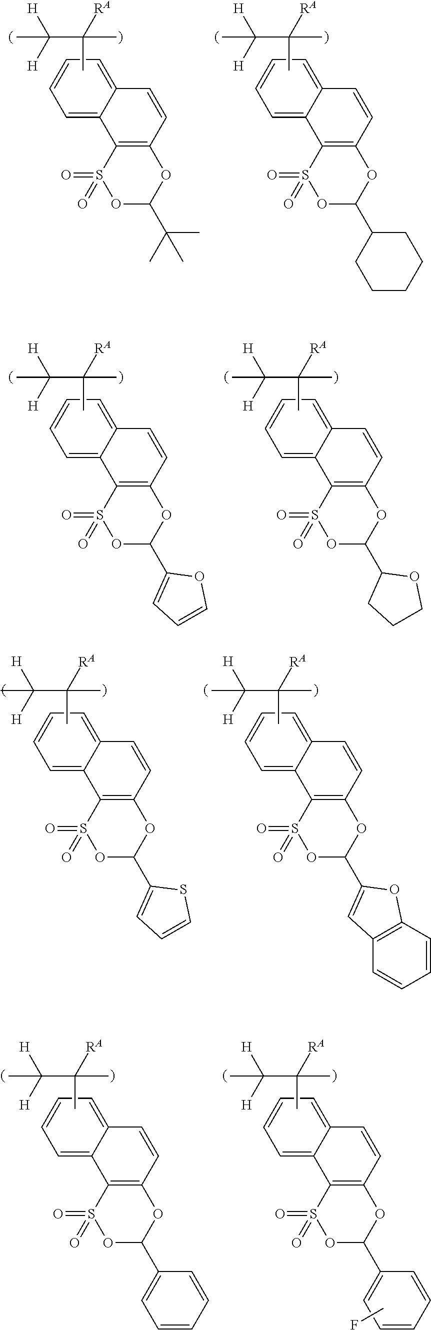

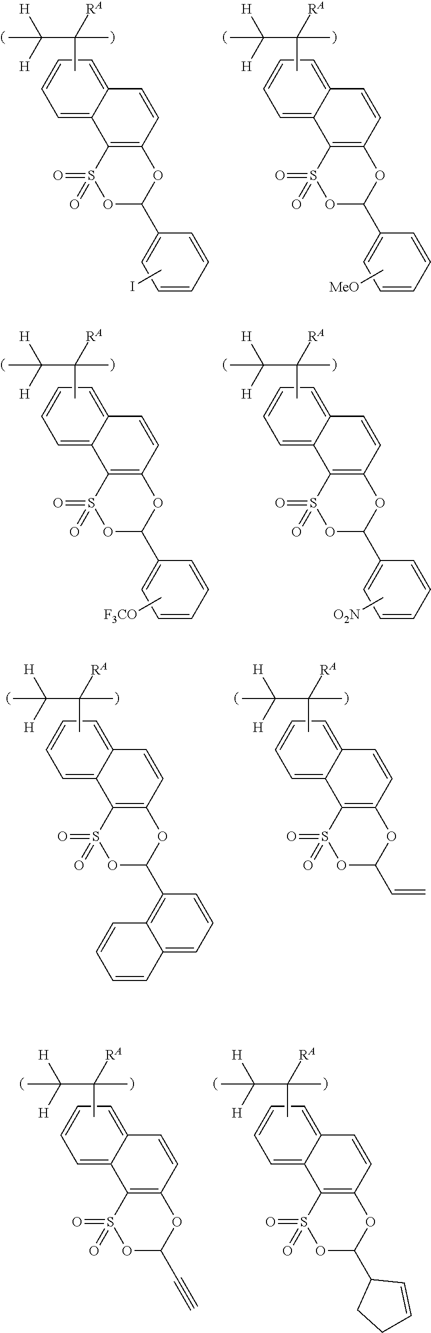

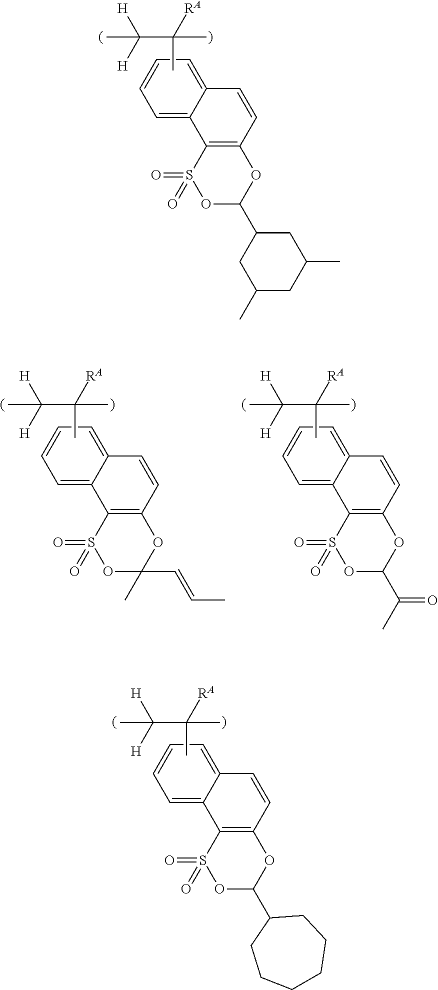

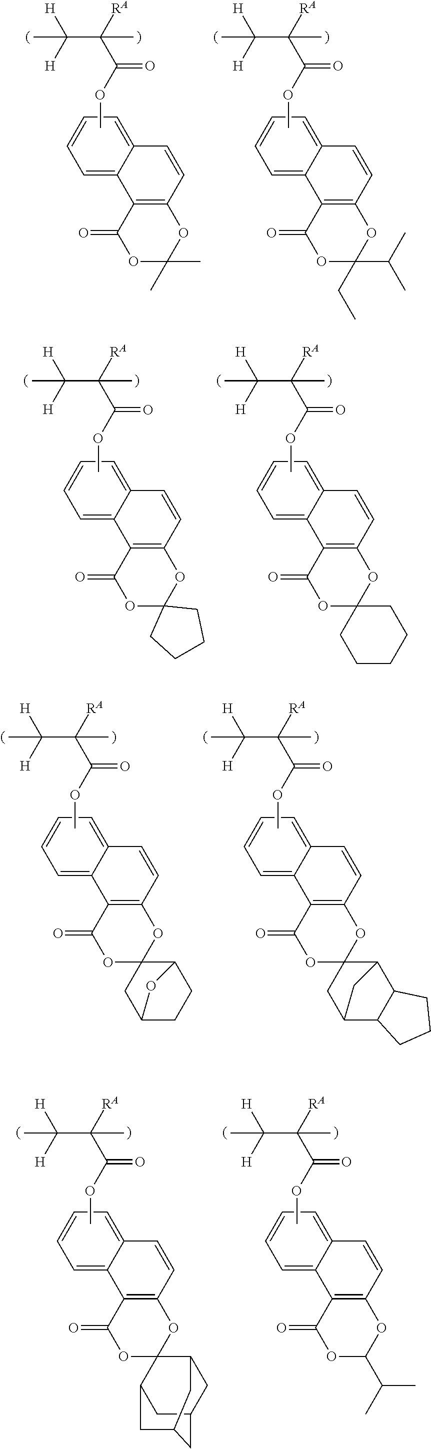

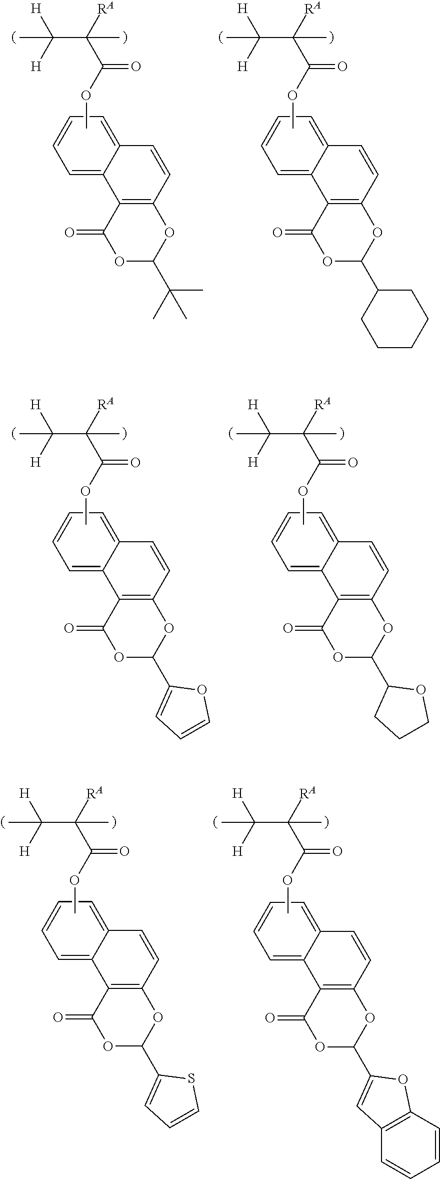

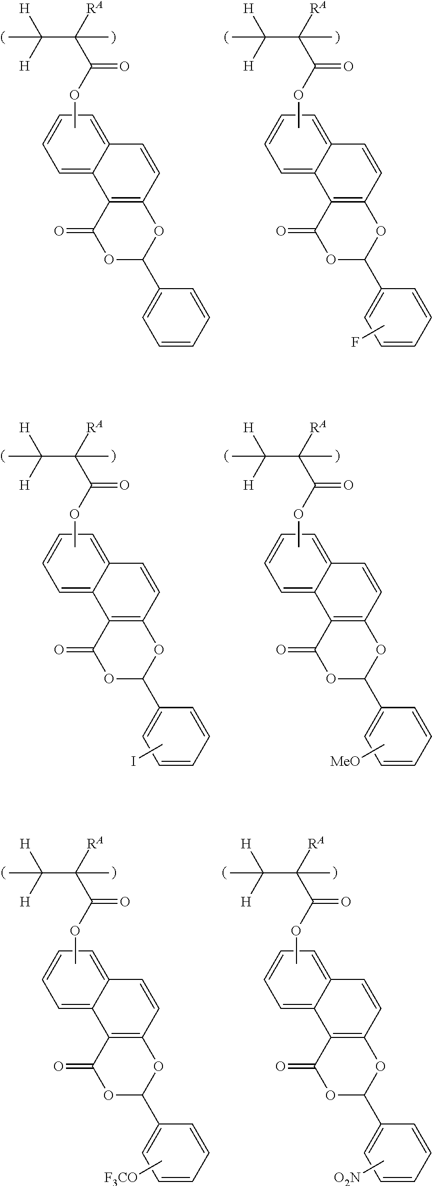

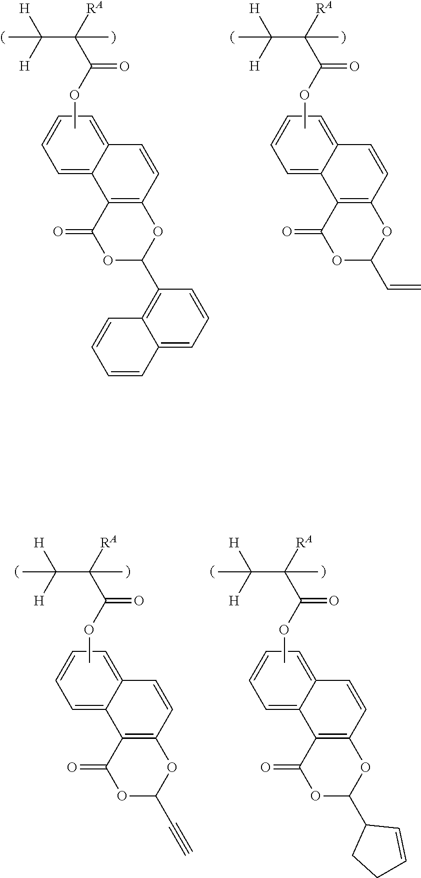

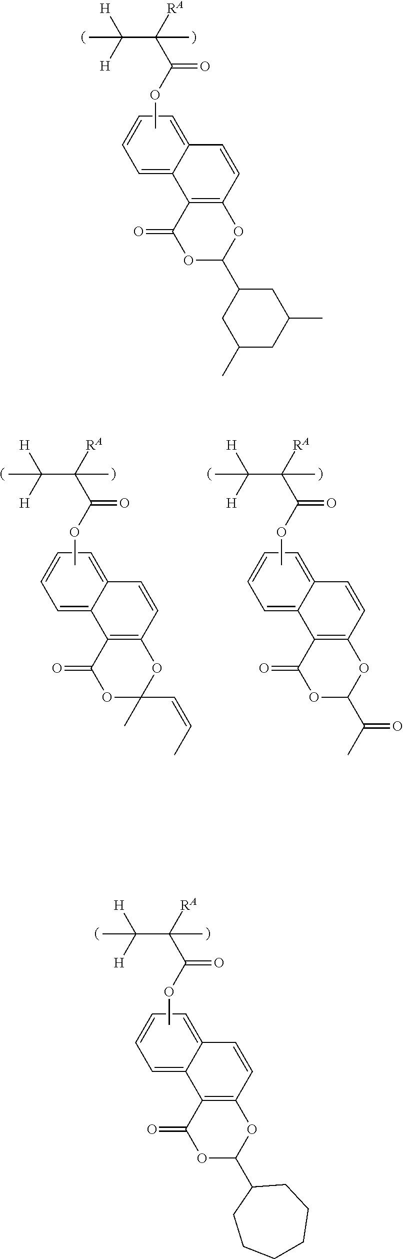

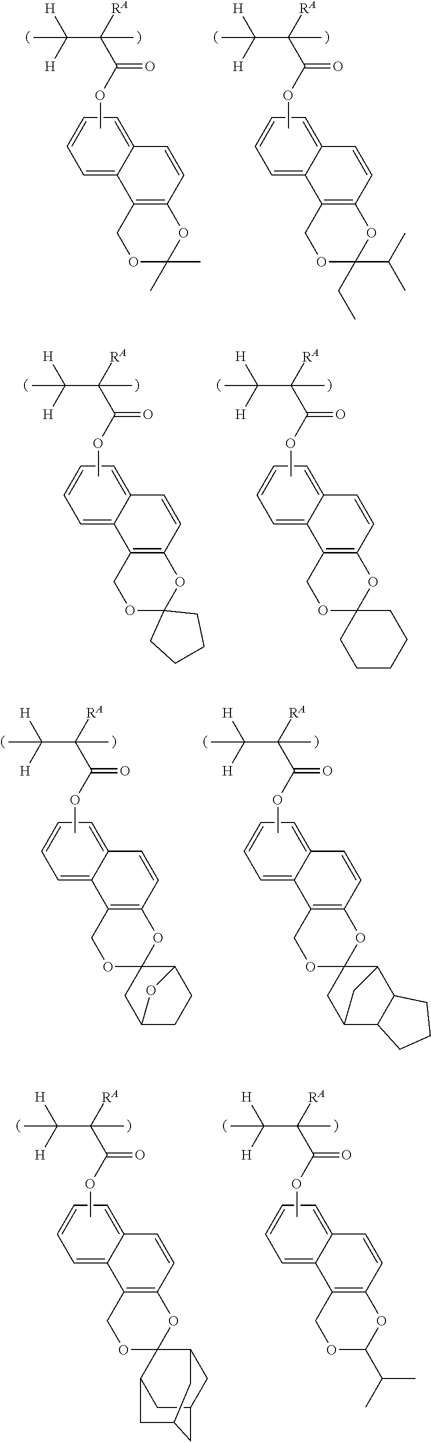

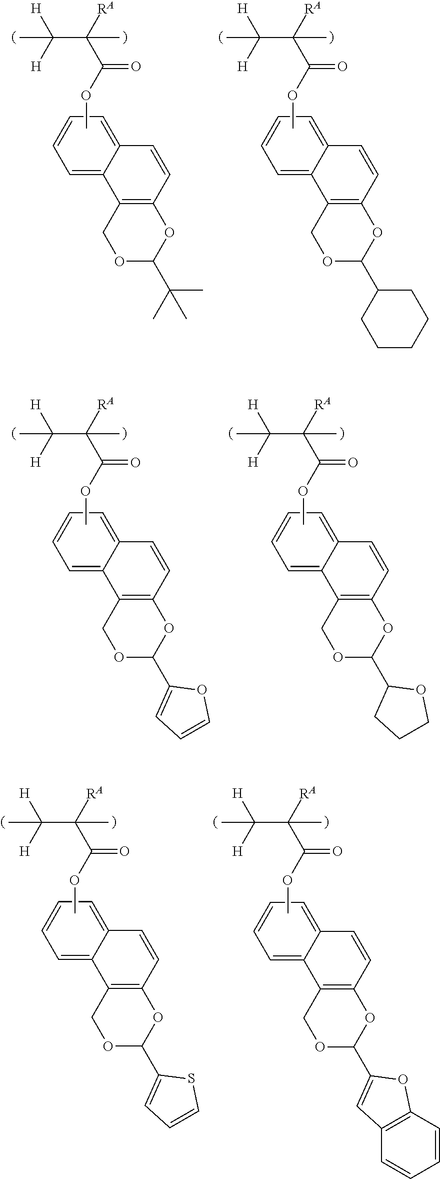

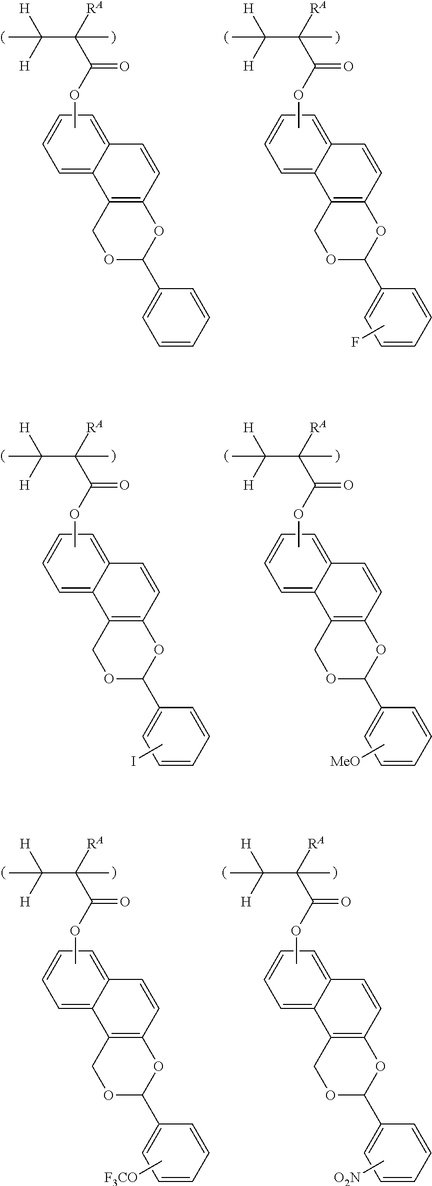

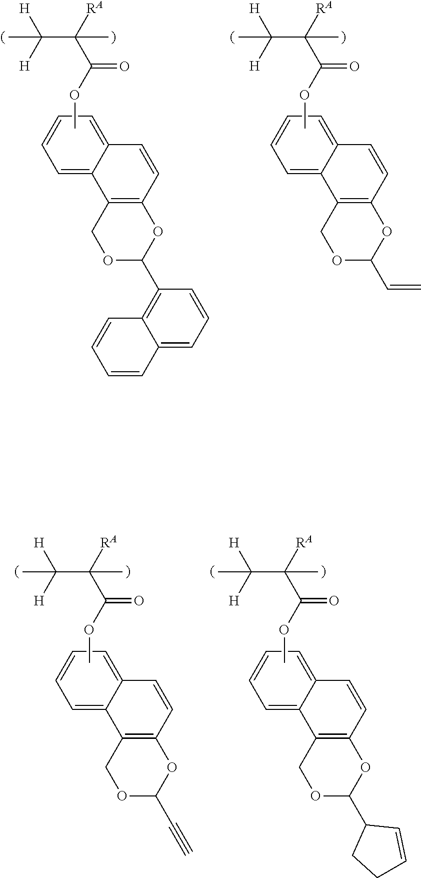

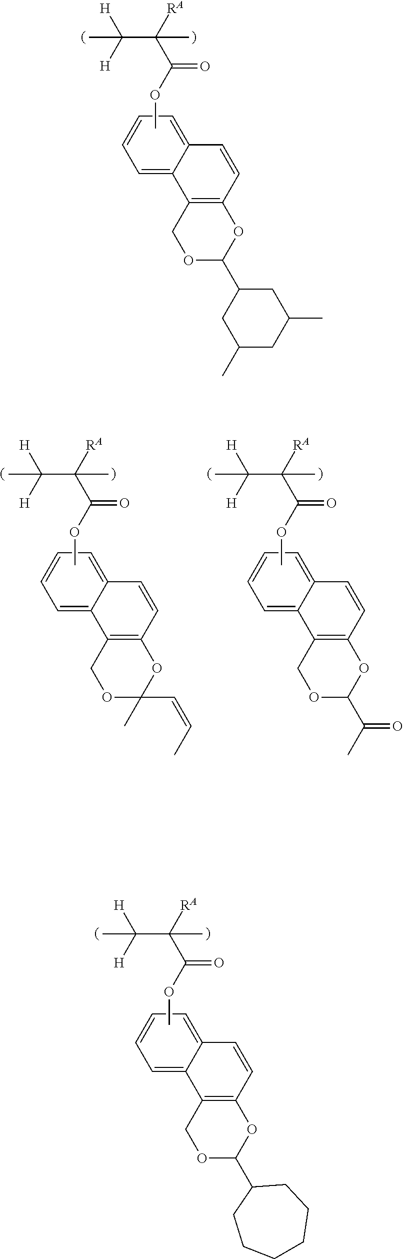

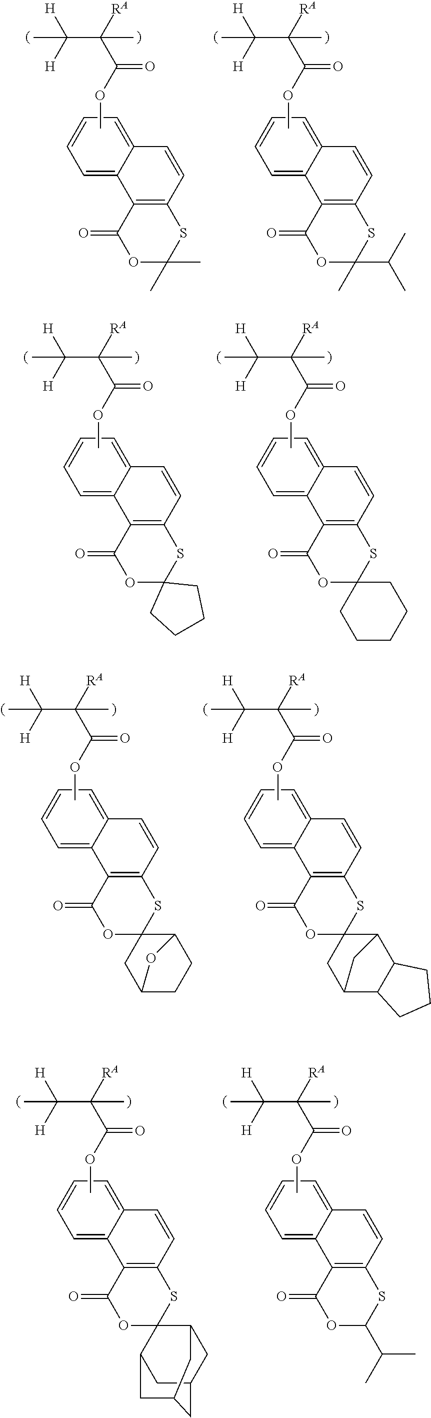

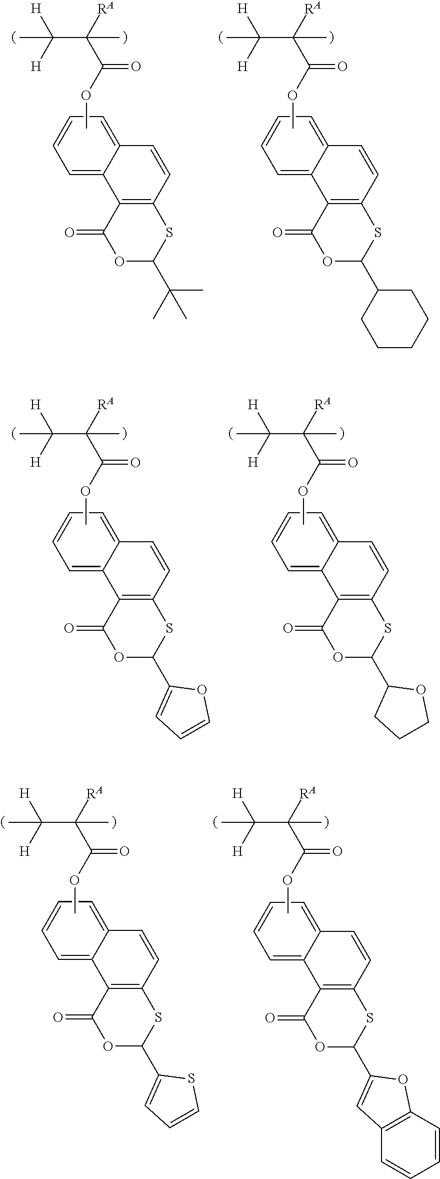

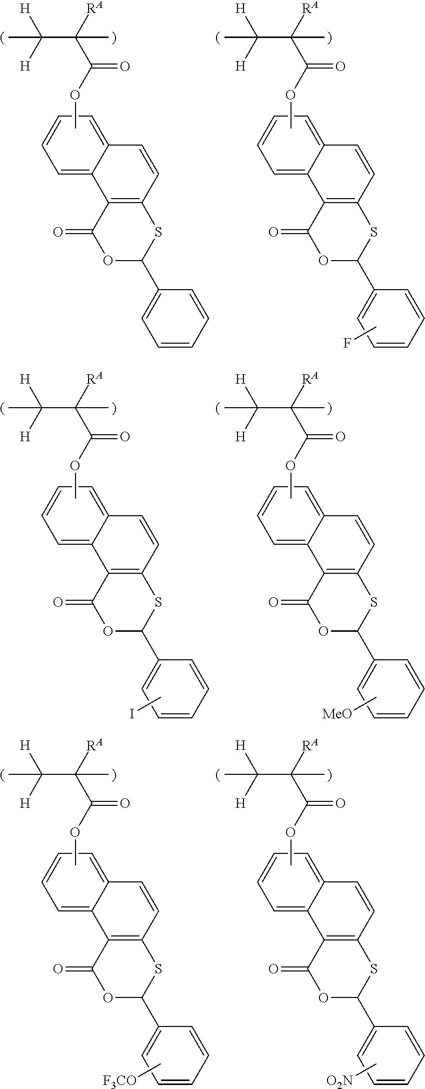

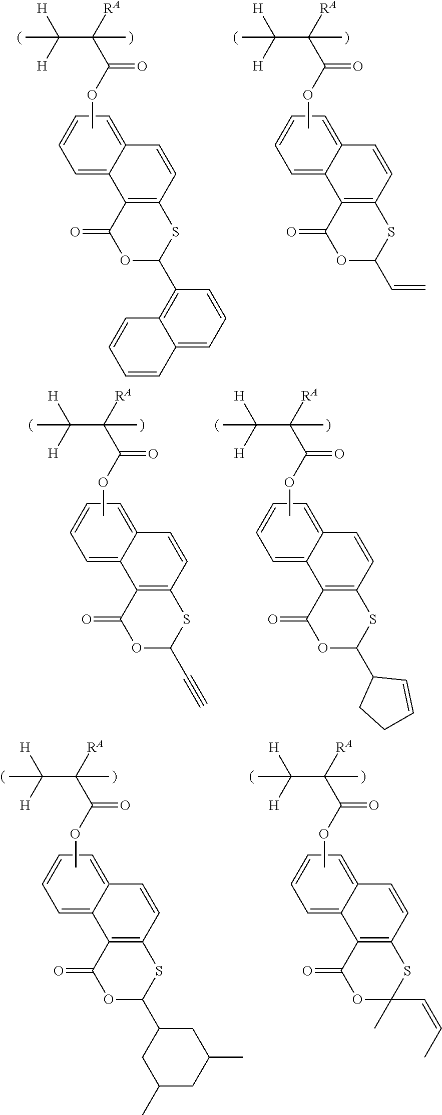

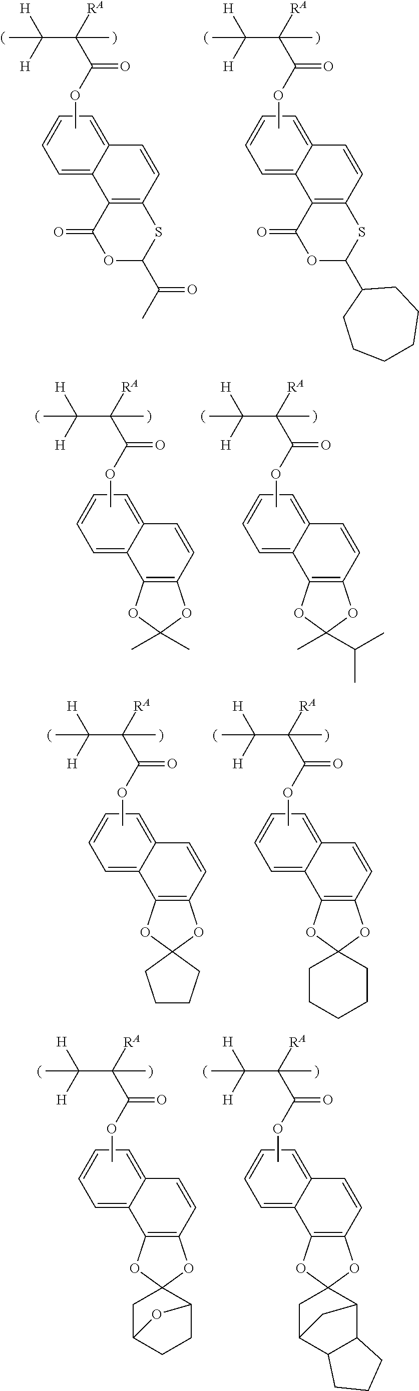

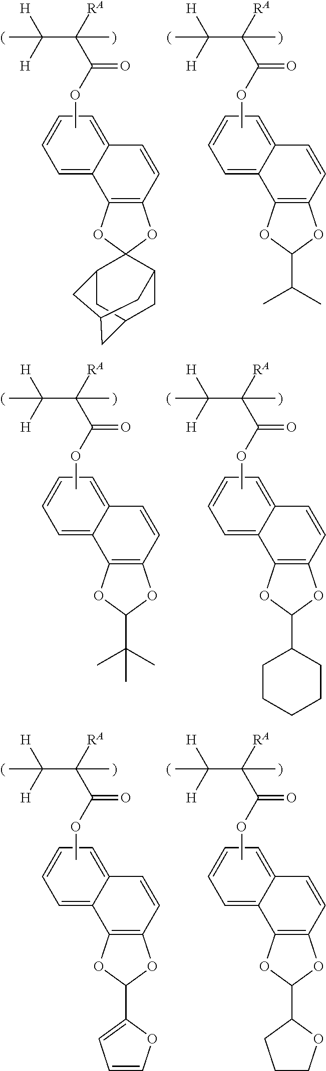

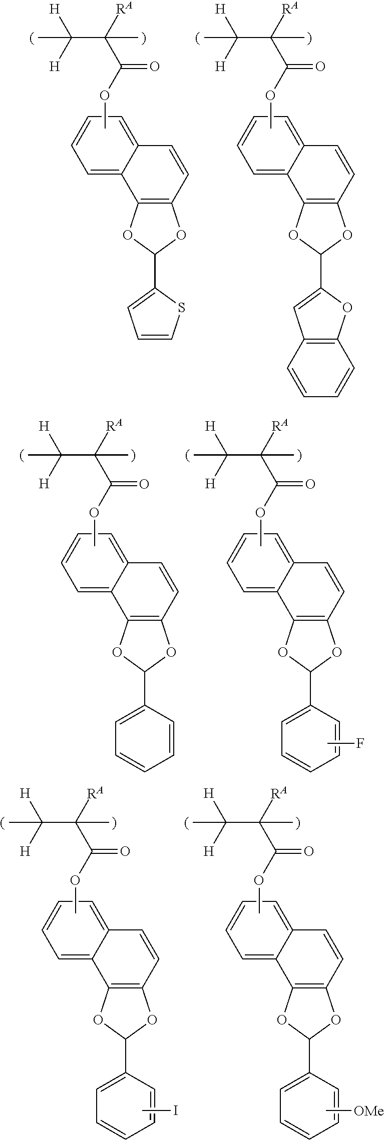

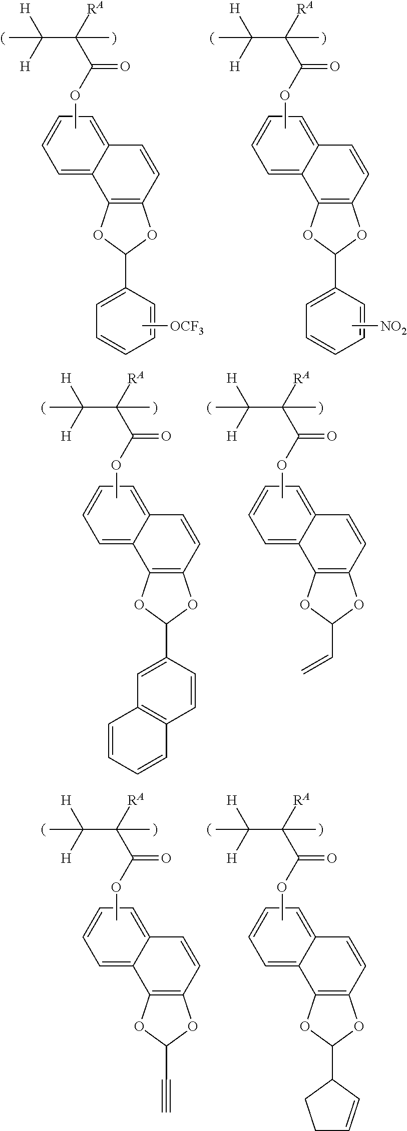

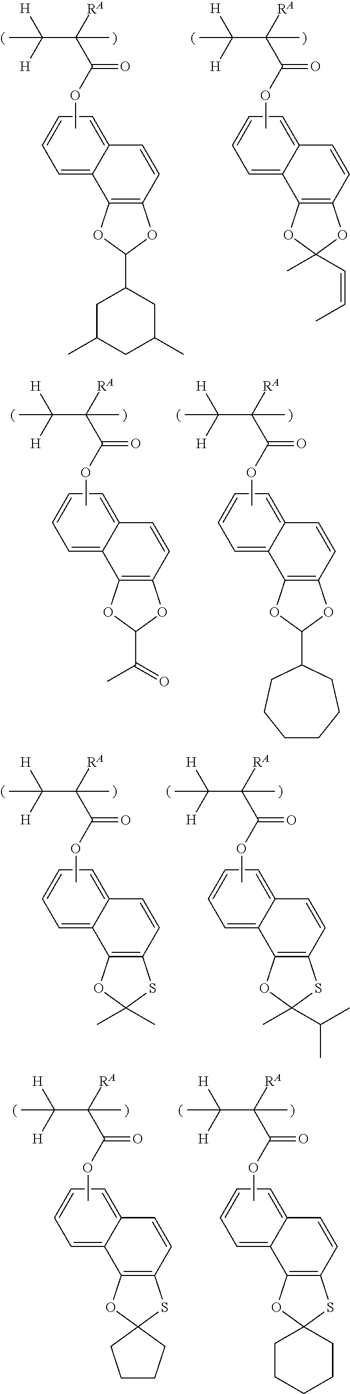

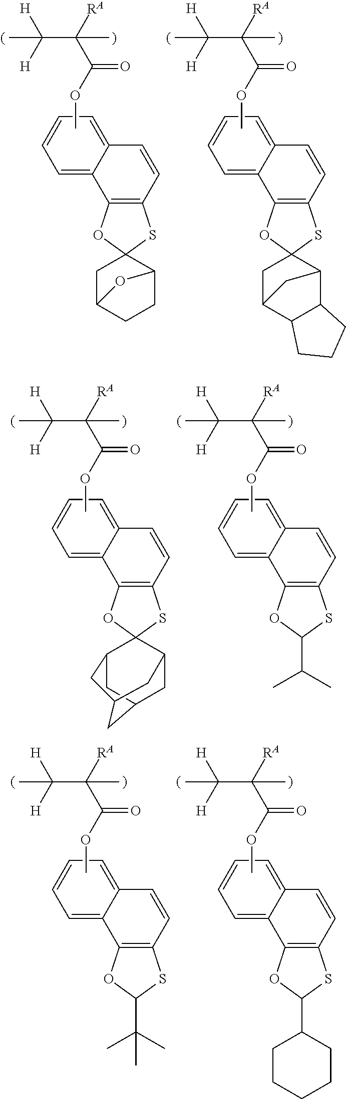

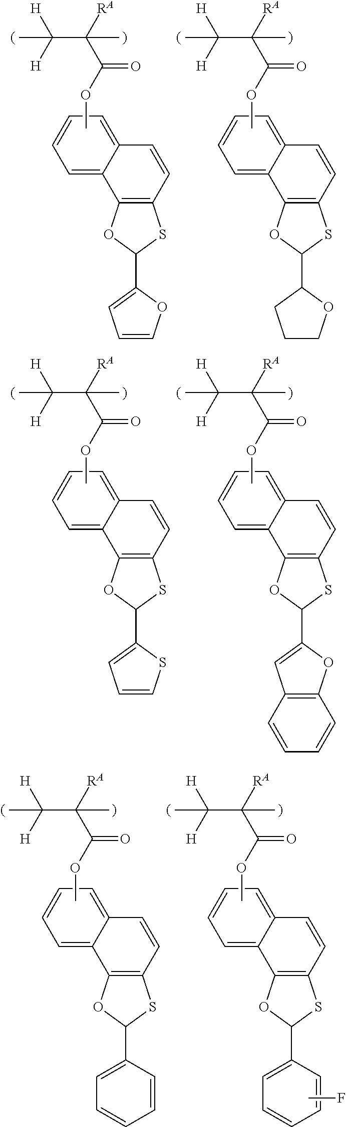

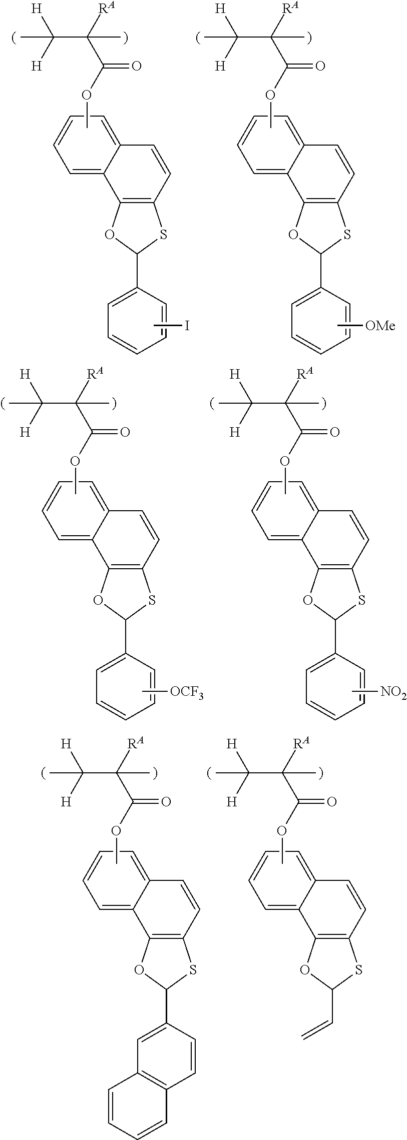

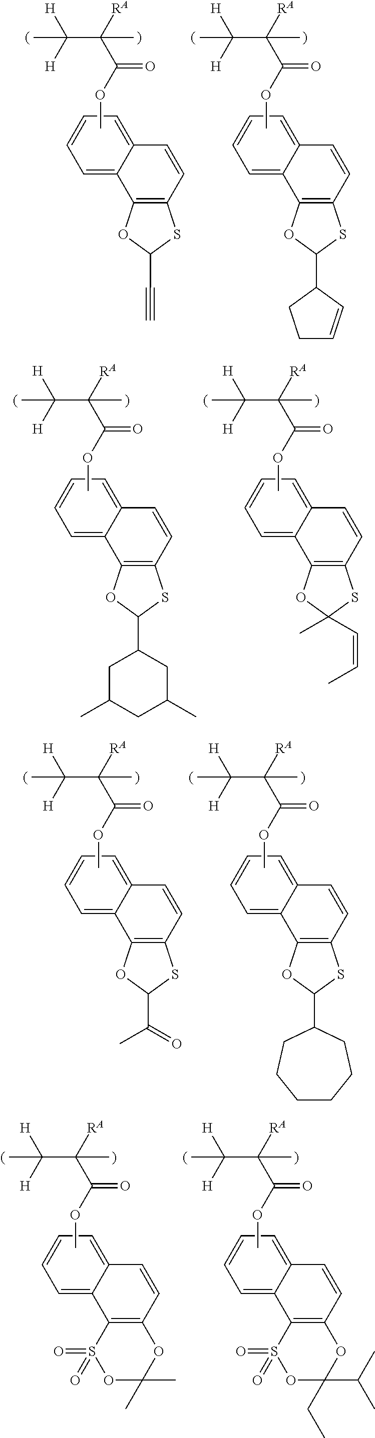

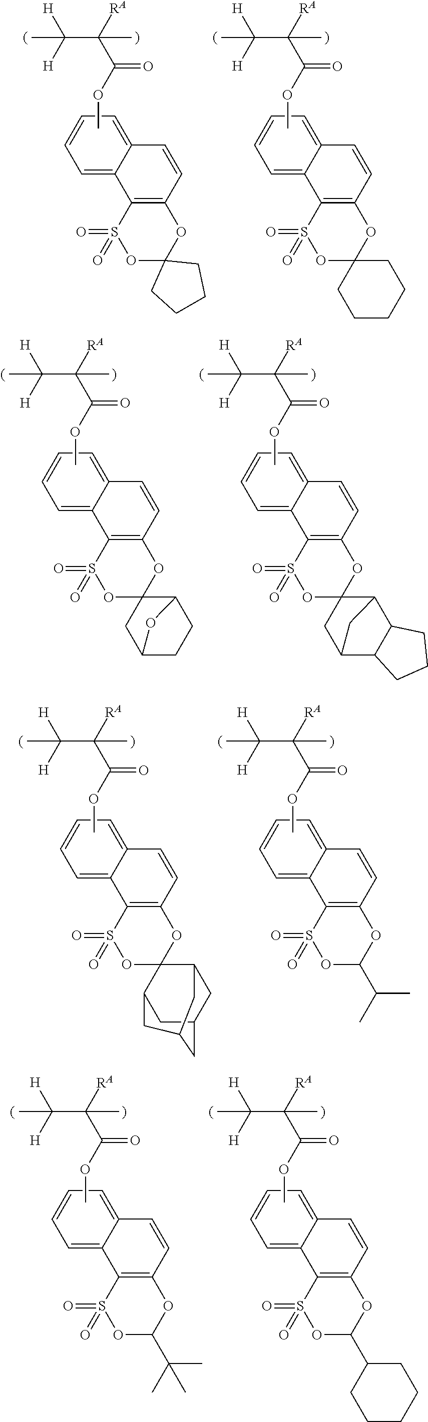

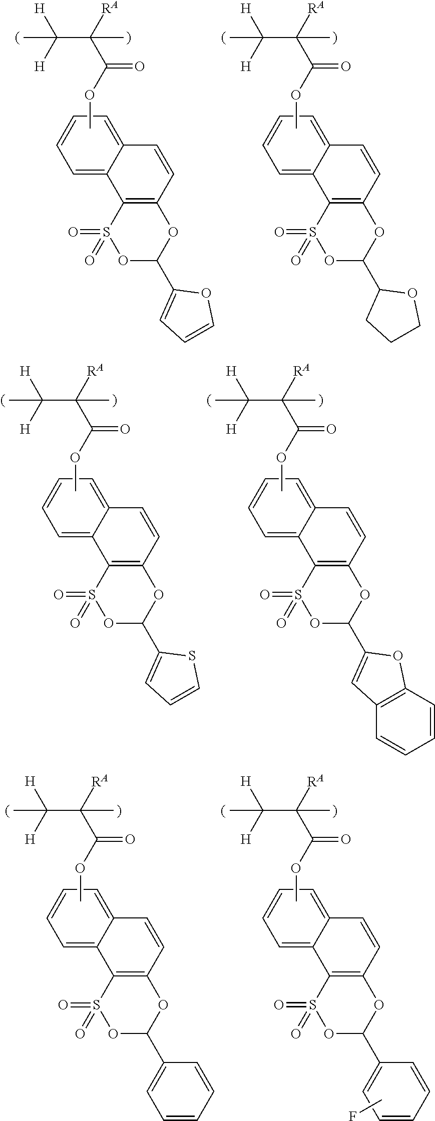

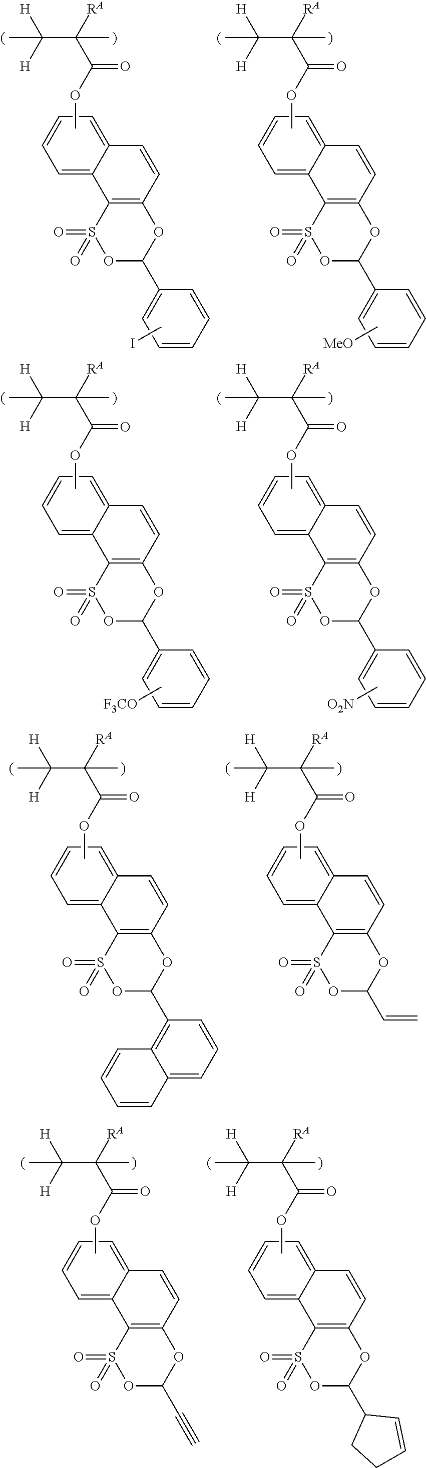

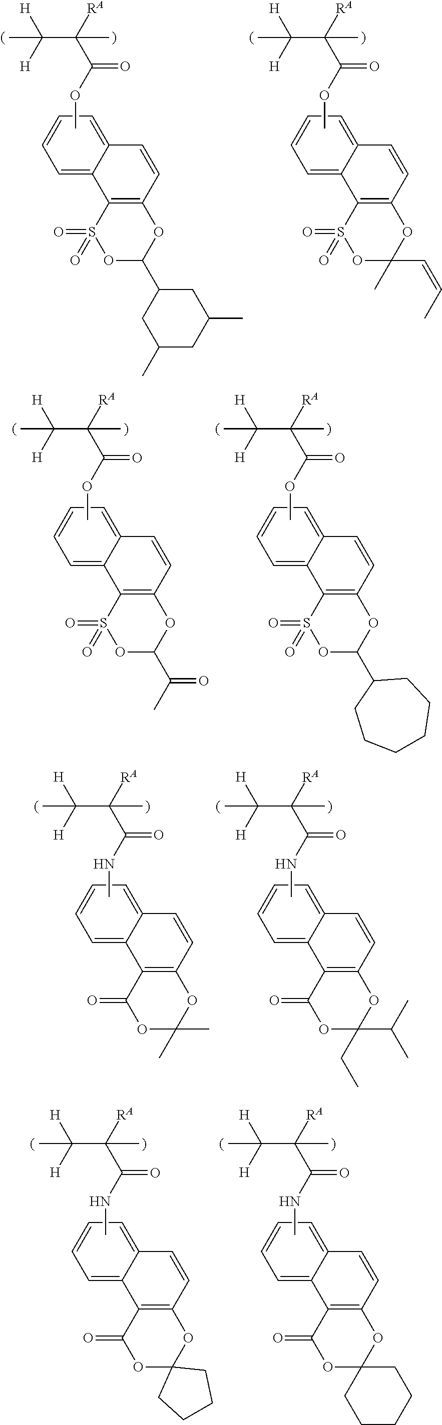

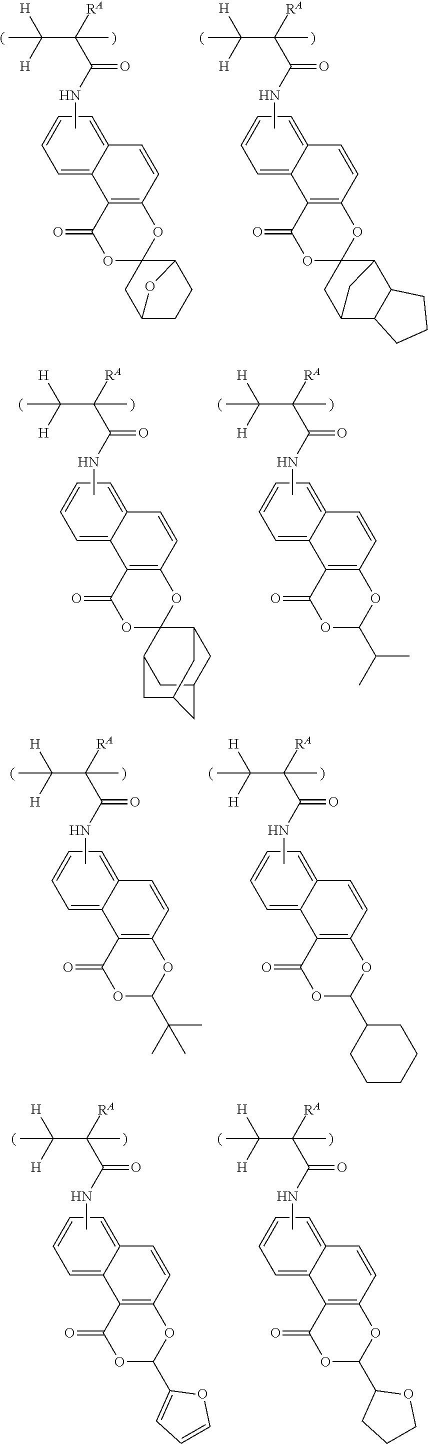

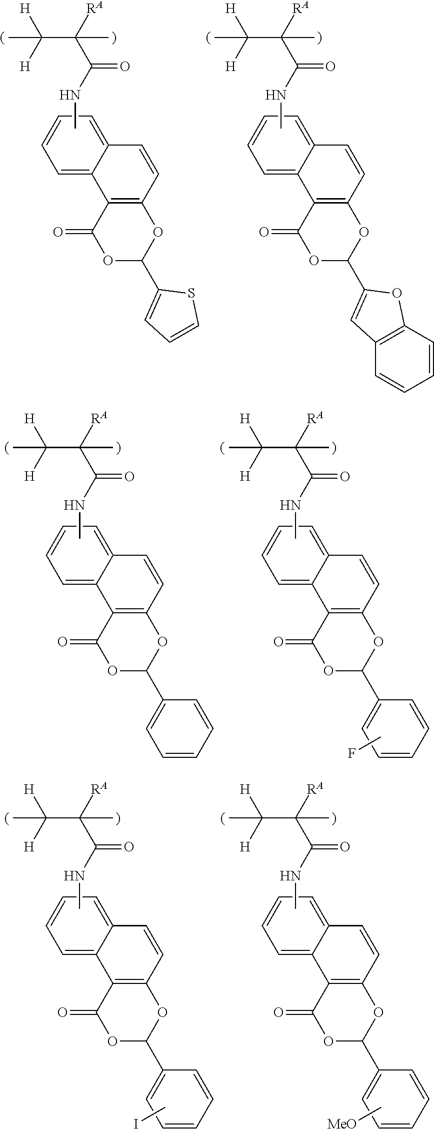

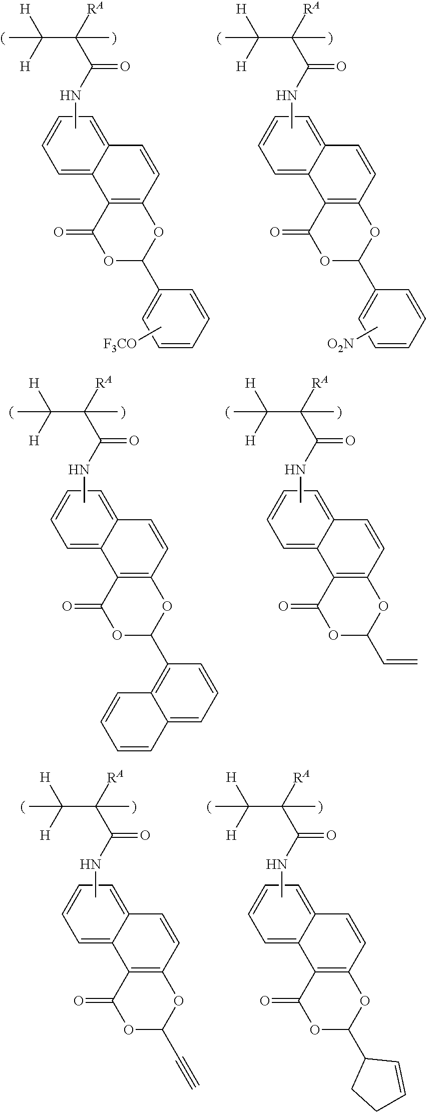

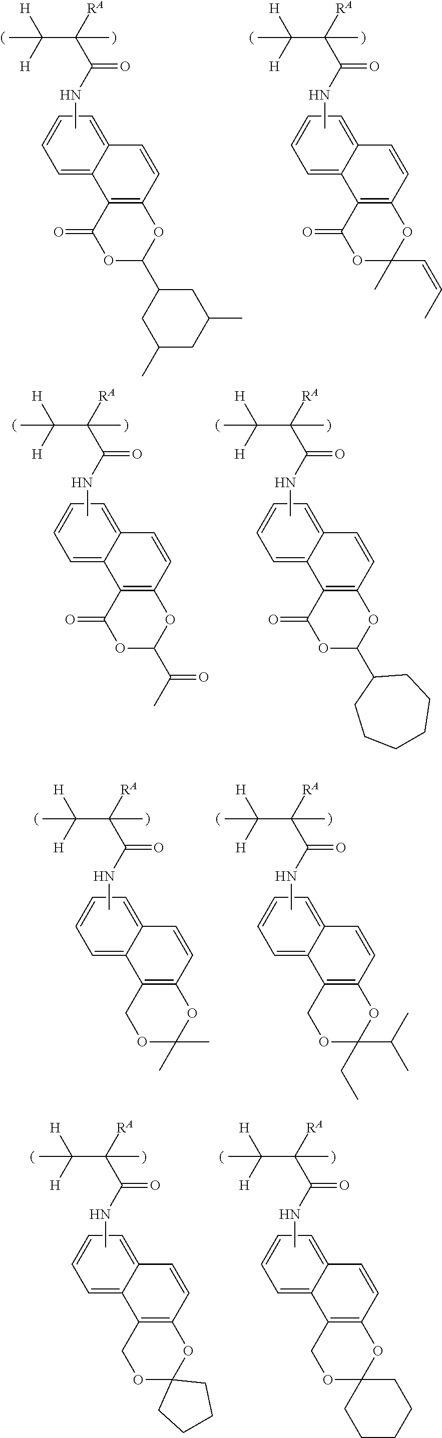

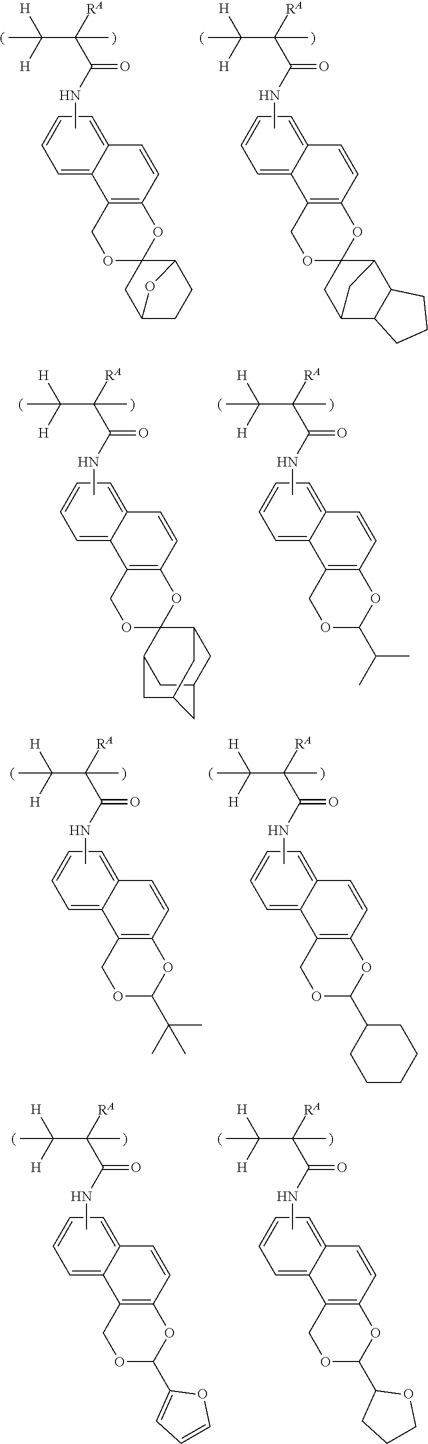

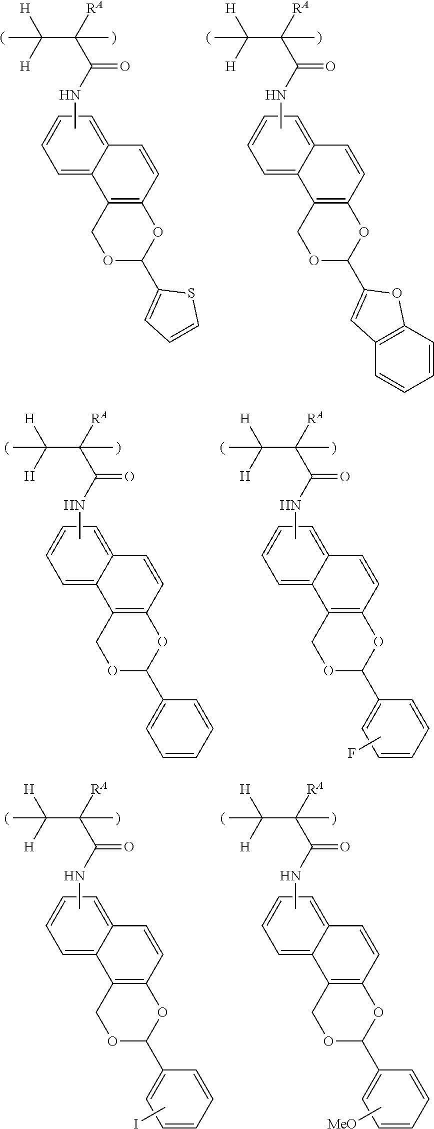

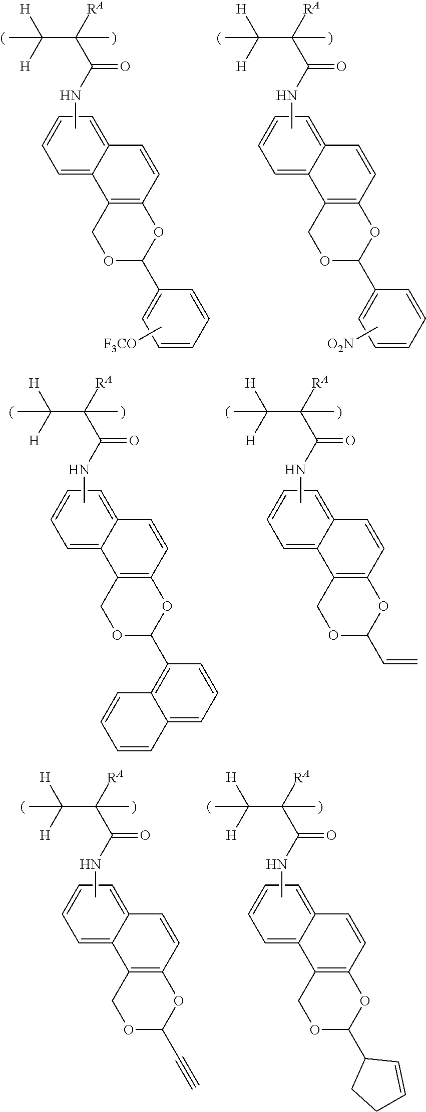

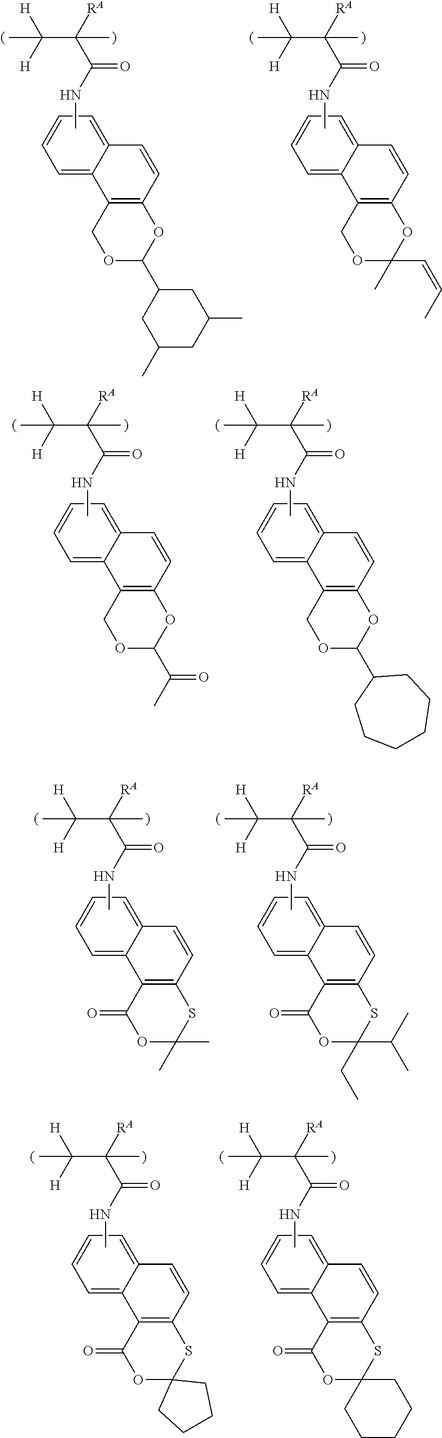

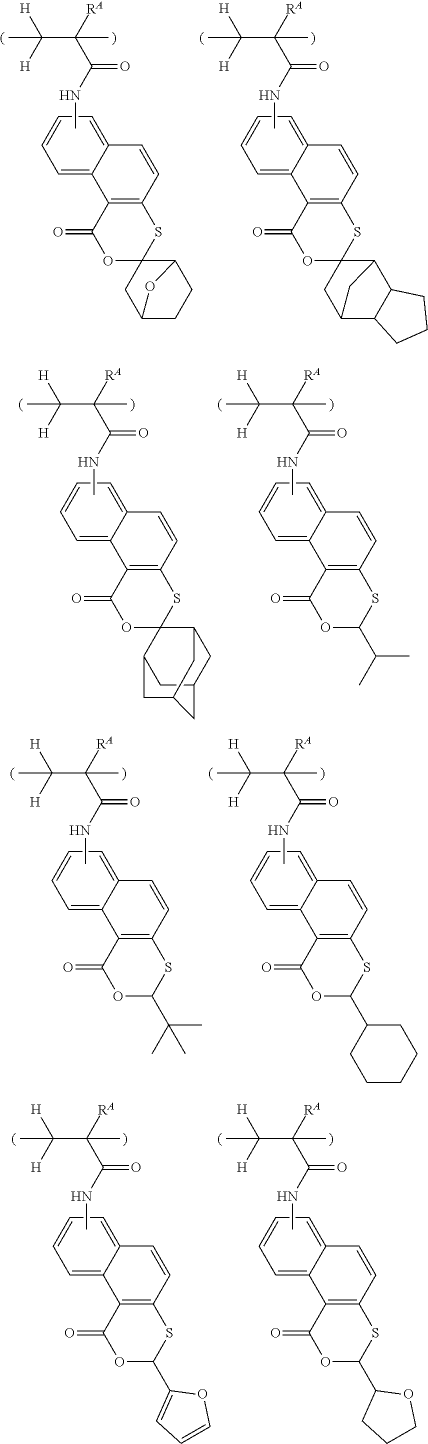

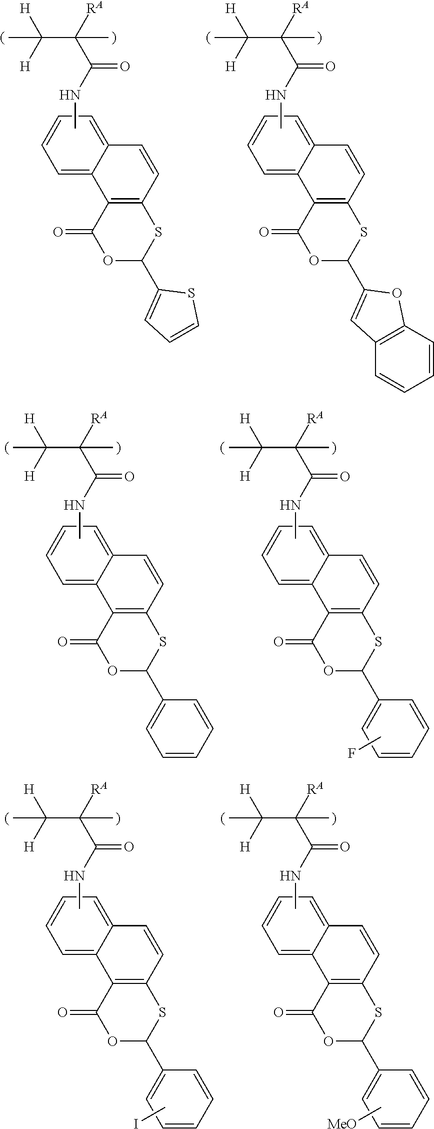

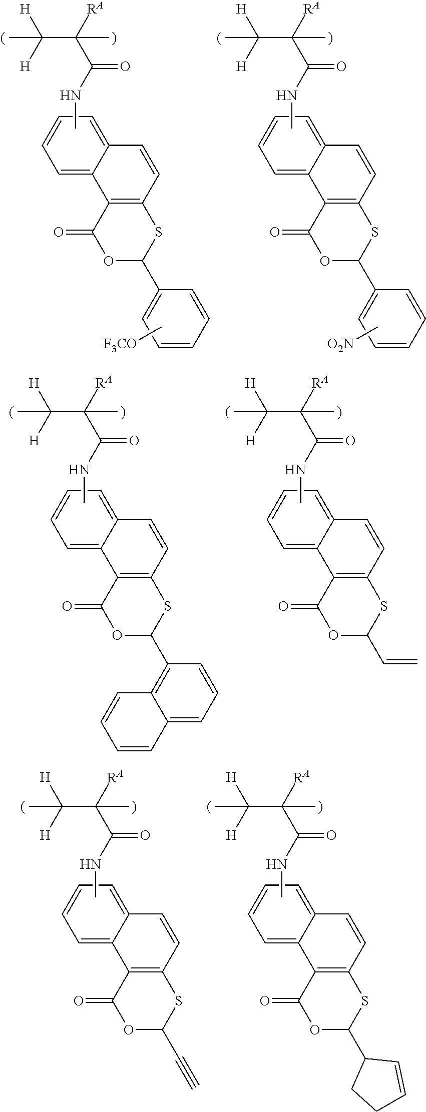

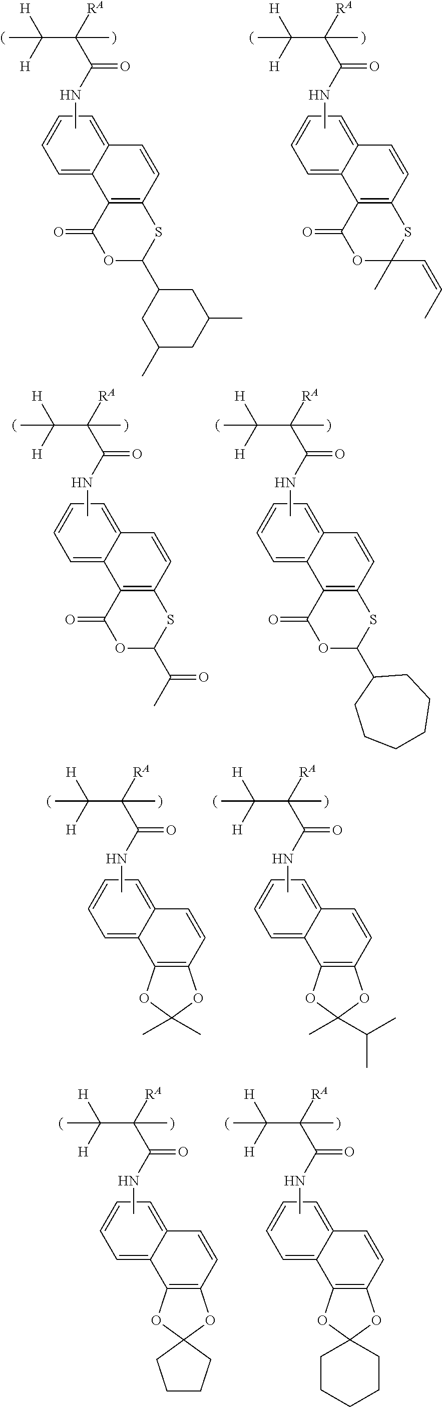

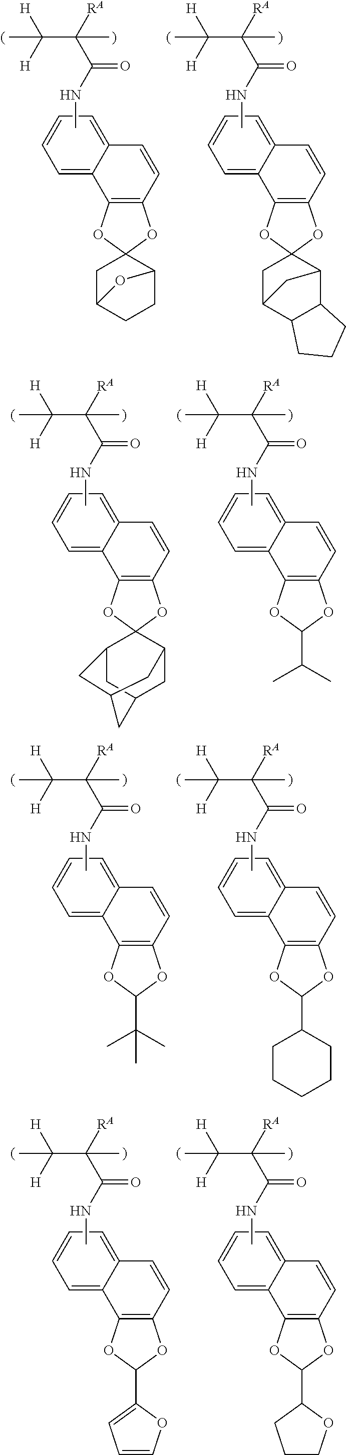

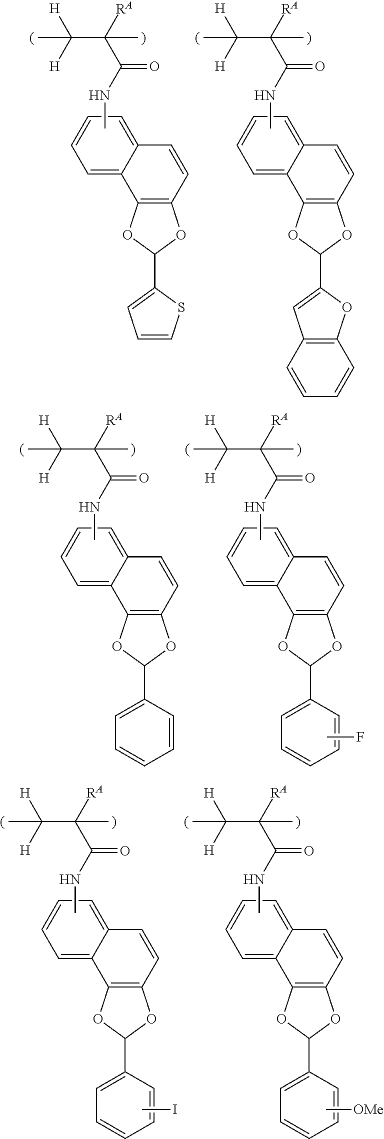

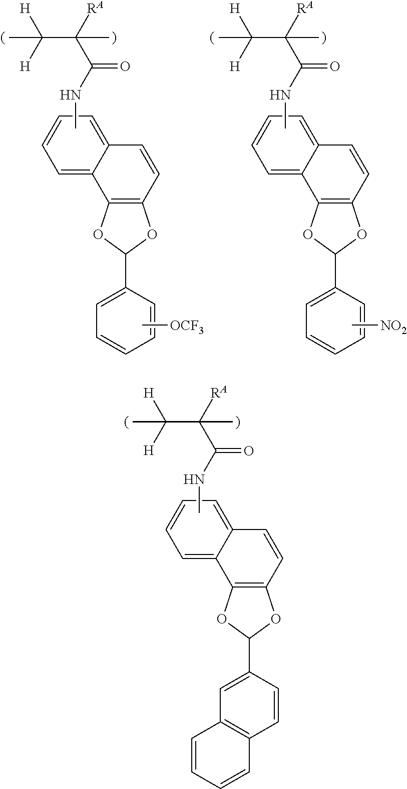

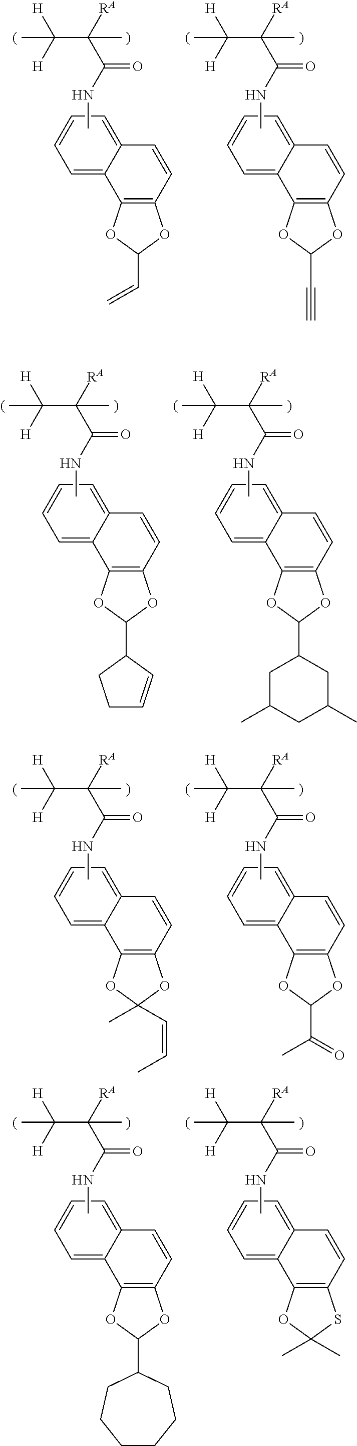

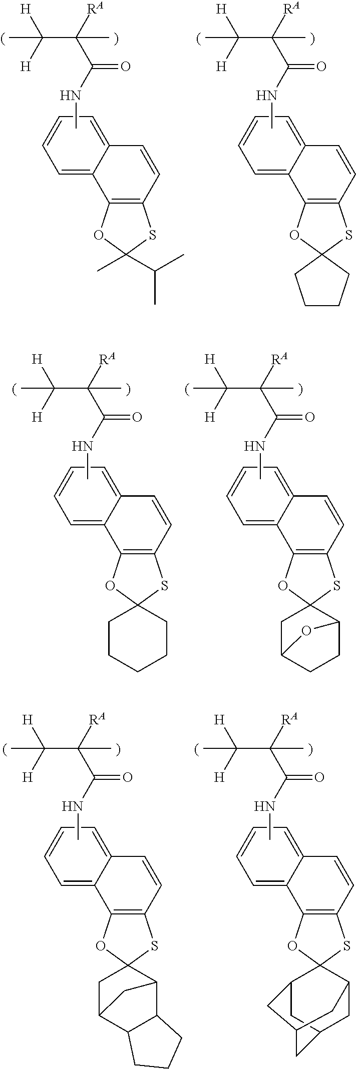

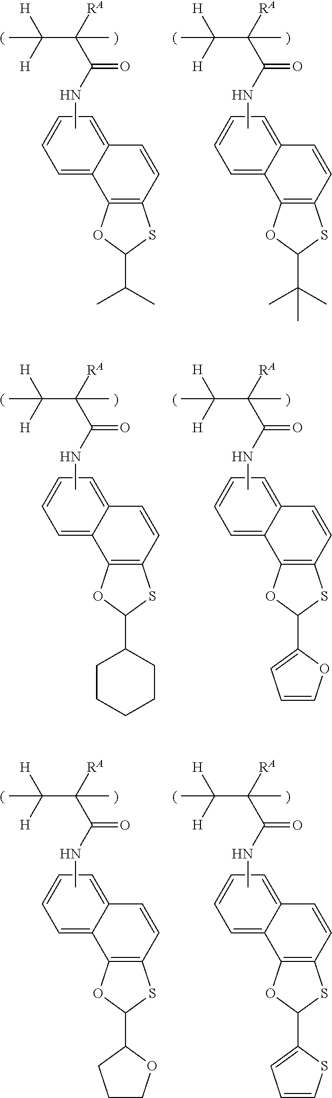

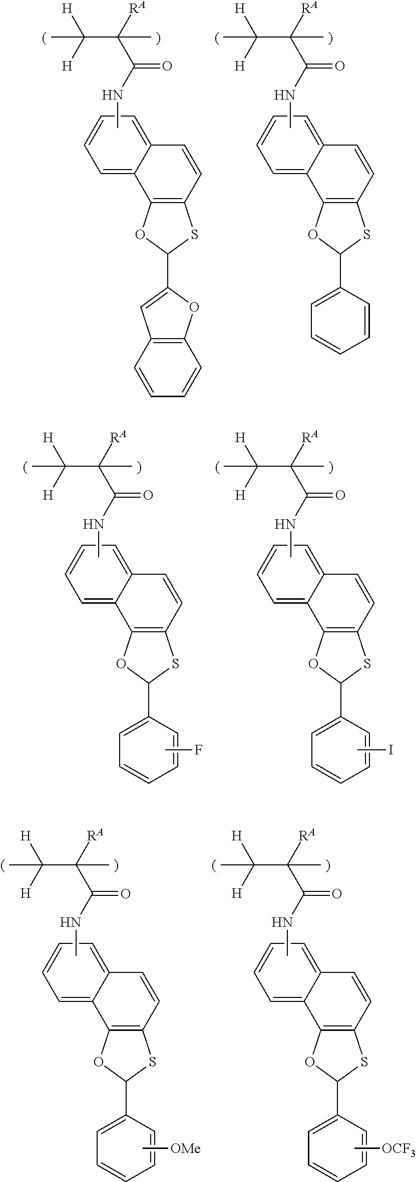

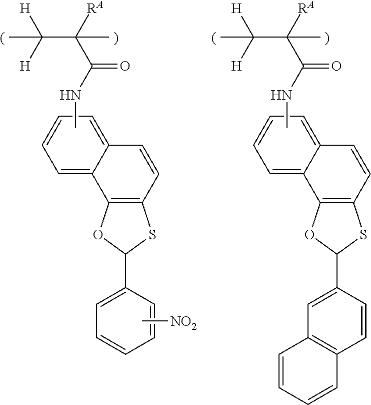

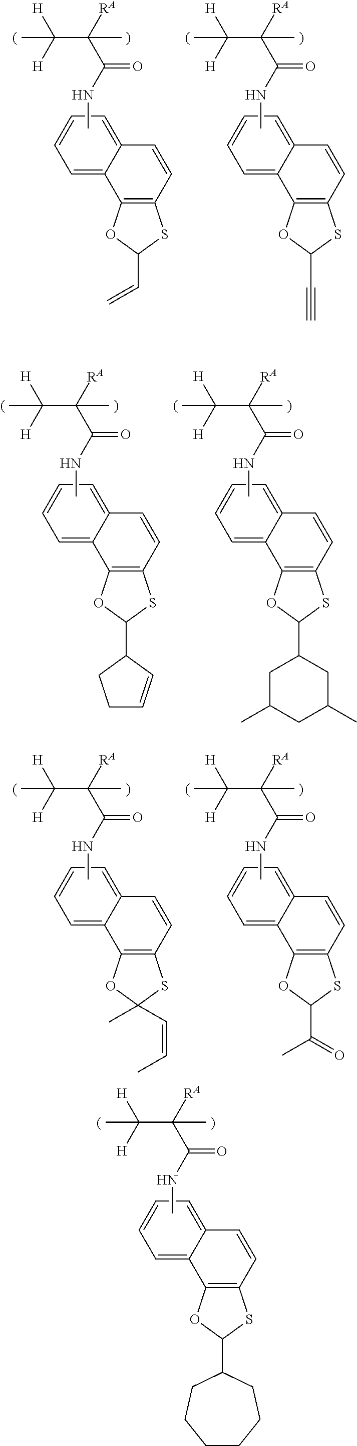

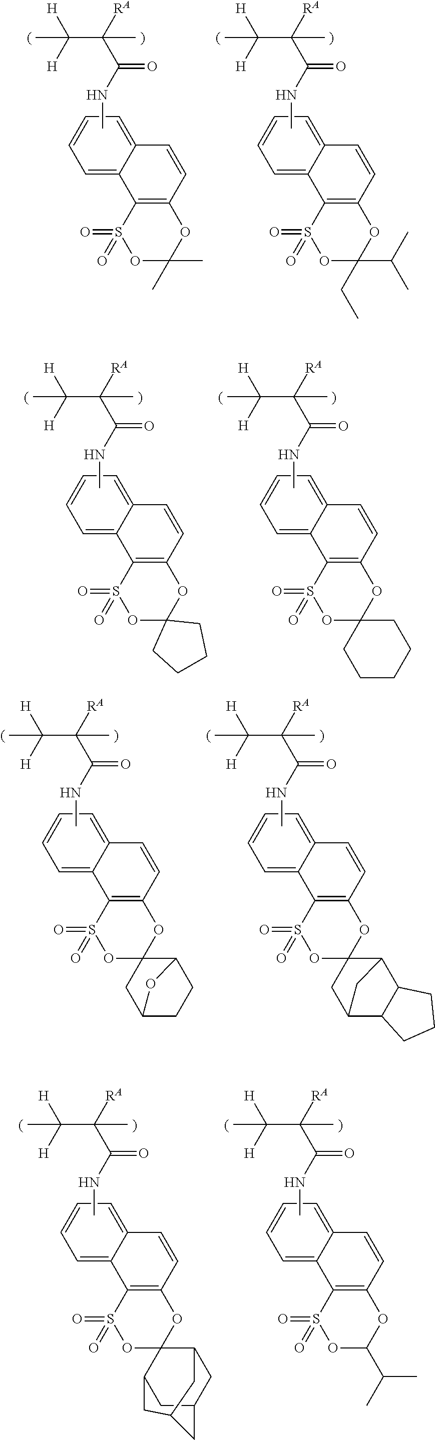

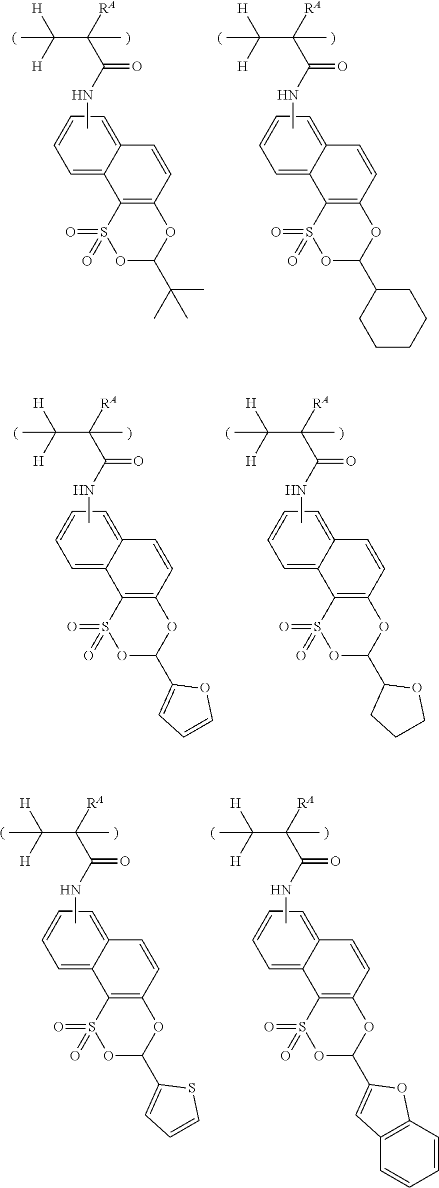

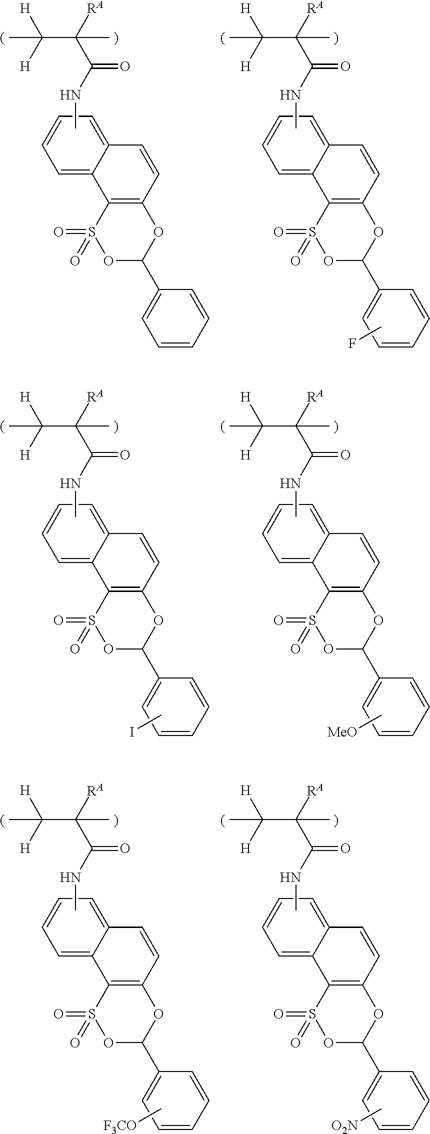

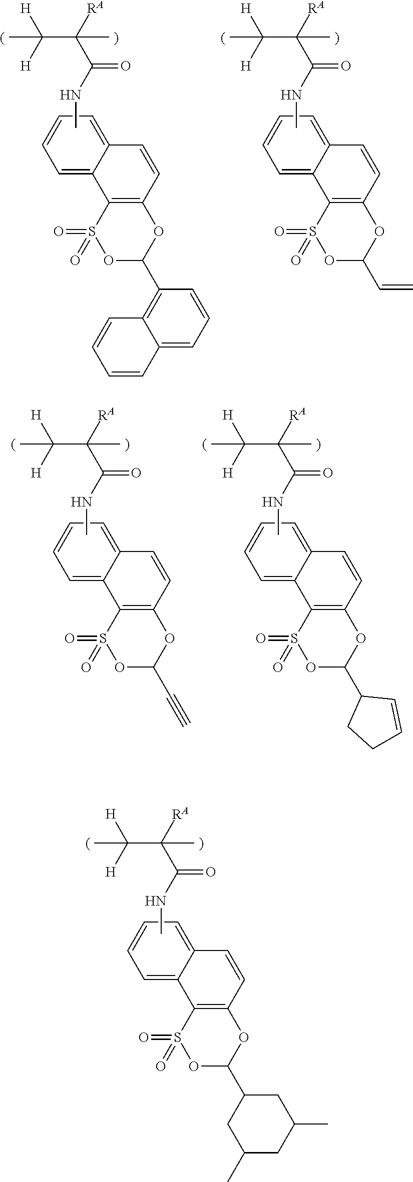

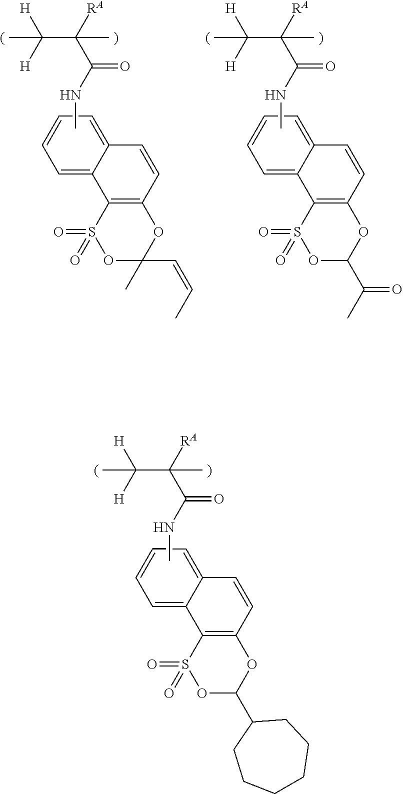

The base polymer preferably further contains a repeating unit represented by formula (b1) (hereinafter referred to as repeating unit bl) or a repeating unit represented by formula (b2) (hereinafter referred to as repeating unit b2):

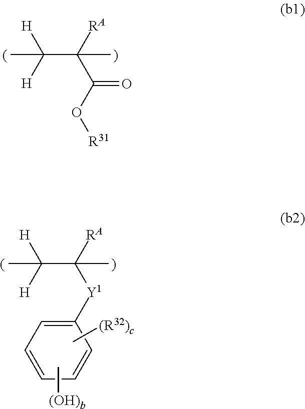

wherein each R^{A} is independently a hydrogen atom, a fluorine atom, a methyl group, or a trifluoromethyl group;
Y¹ is a single bond or *—C(═O)—O—, and “*” represents a bond to a carbon atom of a main chain;
R³¹ is a hydrogen atom or a group having 1 to 20 carbon atoms and comprising at least one structure selected from the group consisting of a hydroxy group other than a phenolic hydroxy group, a cyano group, a carbonyl group, a carboxy group, an ether bond, an ester bond, a sulfonate ester bond, a carbonate bond, a lactone ring, a sultone ring, and a carboxylic anhydride (—C(═O)—O—C(═O)—);
R³² is a halogen atom, a carboxy group, a nitro group, a cyano group, an alkoxycarbonyl group, a hydrocarbyl group having 1 to 20 carbon atoms and optionally comprising a heteroatom, a hydrocarbyloxy group having 1 to 20 carbon atoms and optionally comprising a heteroatom, a hydrocarbylcarbonyl group having 2 to 20 carbon atoms and optionally comprising a heteroatom, a hydrocarbylcarbonyloxy group having 2 to 20 carbon atoms and optionally comprising a heteroatom, or a hydrocarbyloxycarbonyl group having 2 to 20 carbon atoms and optionally comprising a heteroatom, wherein when “c” is 2 or more, R³² are the same as or different from each other; and
“b” is an integer of 1 to 4, and “c” is an integer of 0 to 4, provided that 1≤b+c≤5.

In formulae(b1) and (b2), each R^{A} is independently a hydrogen atom, a fluorine atom, a methyl group, or a trifluoromethyl group. Y¹ is a single bond or *—C(═O)—O—, and “*” represents a bond to a carbon atom of a main chain. R³¹ is a hydrogen atom or a group having 1 to 20 carbon atoms and comprising at least one structure selected from the group consisting of a hydroxy group other than a phenolic hydroxy group, a cyano group, a carbonyl group, a carboxy group, an ether bond, an ester bond, a sulfonate ester bond, a carbonate bond, a lactone ring, a sultone ring, and a carboxylic anhydride (—C(═O) —O—C(═O)—). R³² is a halogen atom, a carboxy group, a nitro group, a cyano group, an alkoxycarbonyl group, a hydrocarbyl group having 1 to 20 carbon atoms and optionally comprising a heteroatom, a hydrocarbyloxy group having 1 to 20 carbon atoms and optionally comprising a heteroatom, a hydrocarbylcarbonyl group having 2 to 20 carbon atoms and optionally comprising a heteroatom, a hydrocarbylcarbonyloxy group having 2 to 20 carbon atoms and optionally comprising a heteroatom, or a hydrocarbyloxycarbonyl group having 2 to 20 carbon atoms and optionally comprising a heteroatom. “b” is an integer of 1 to 4, and “c” is an integer of 0 to 4, provided that 1≤b+c≤5.

Specific examples of the repeating unit b1 are given below, but not limited thereto. In formulae below, R^{A} is the same as above.

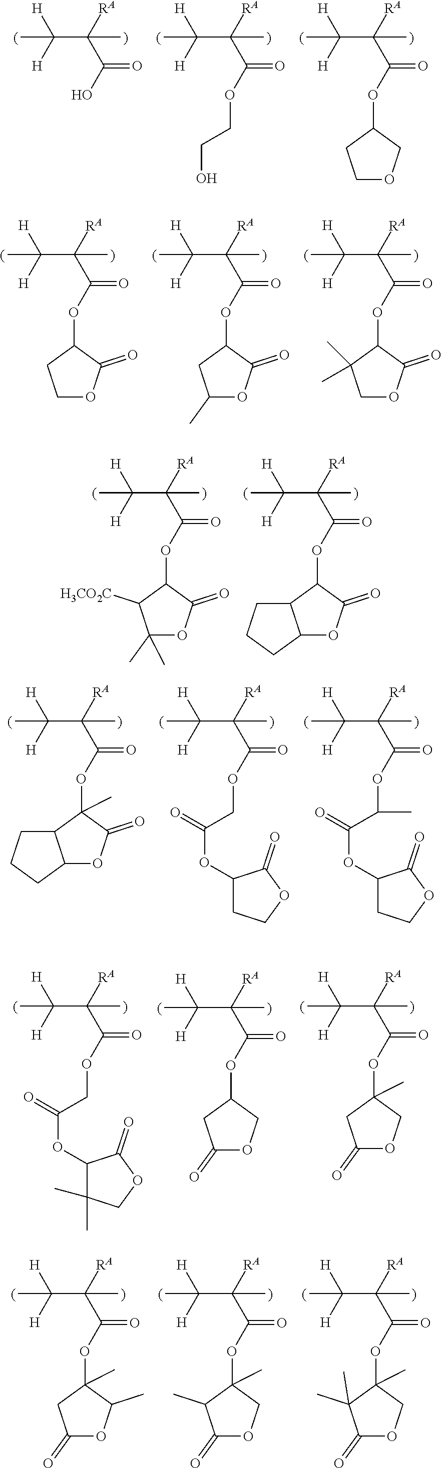

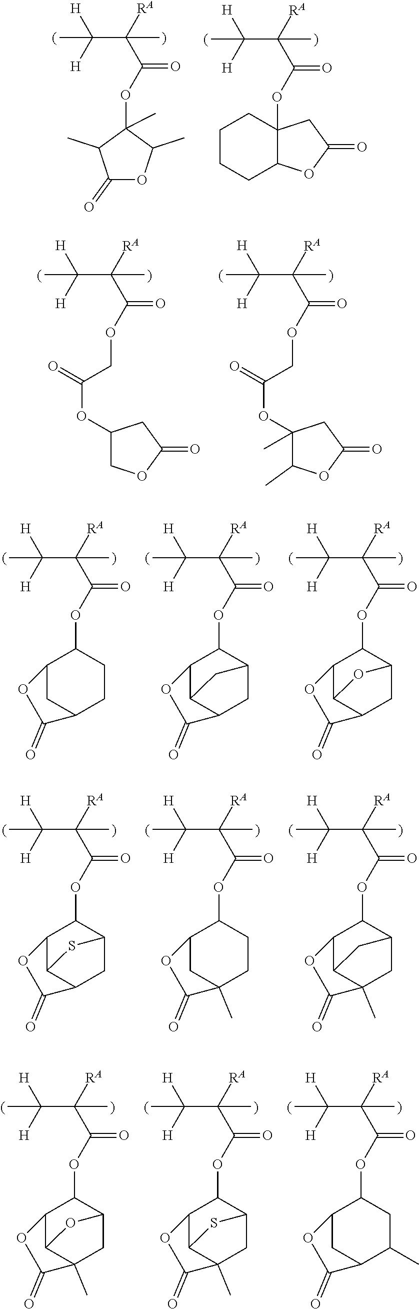

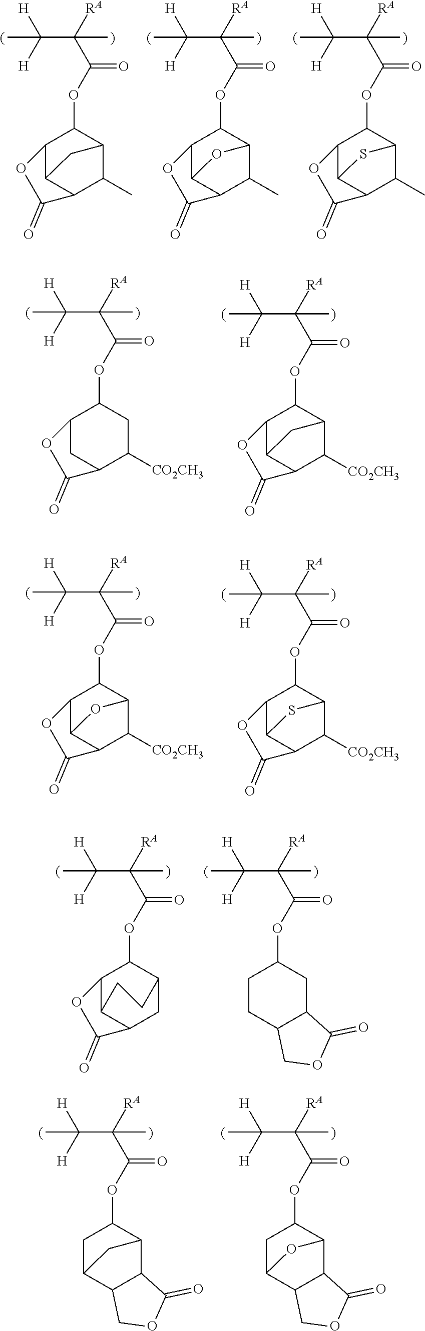

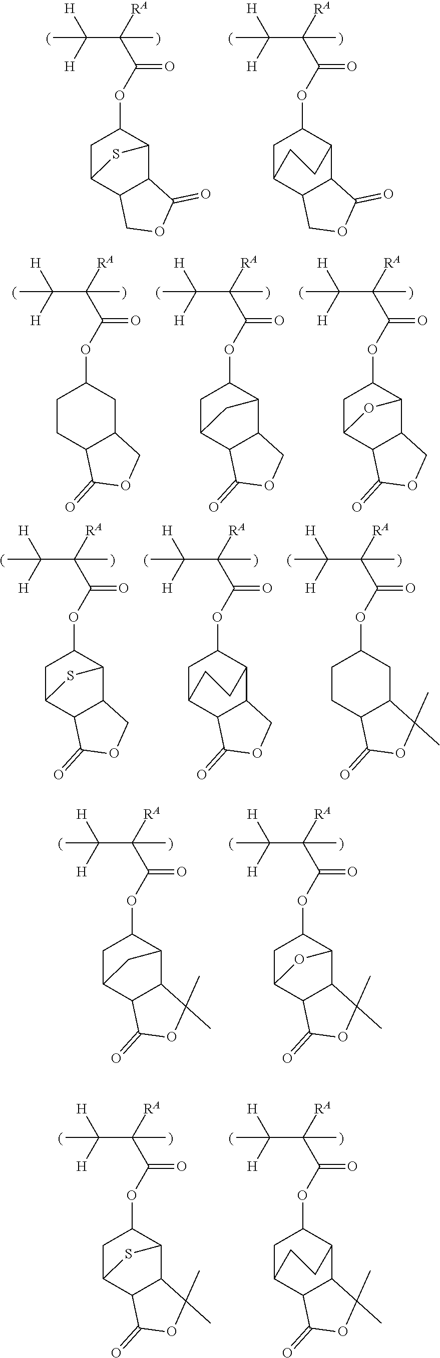

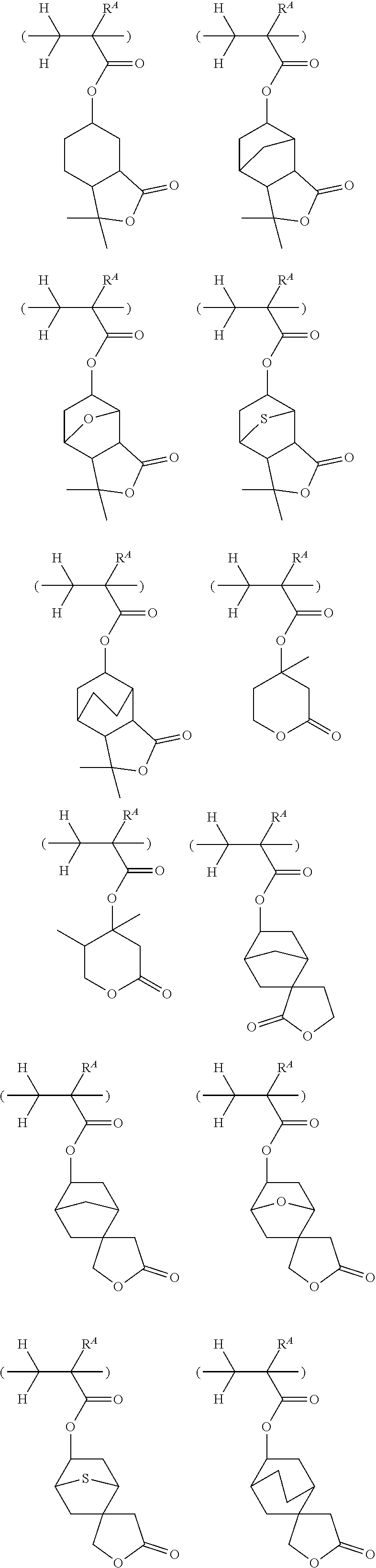

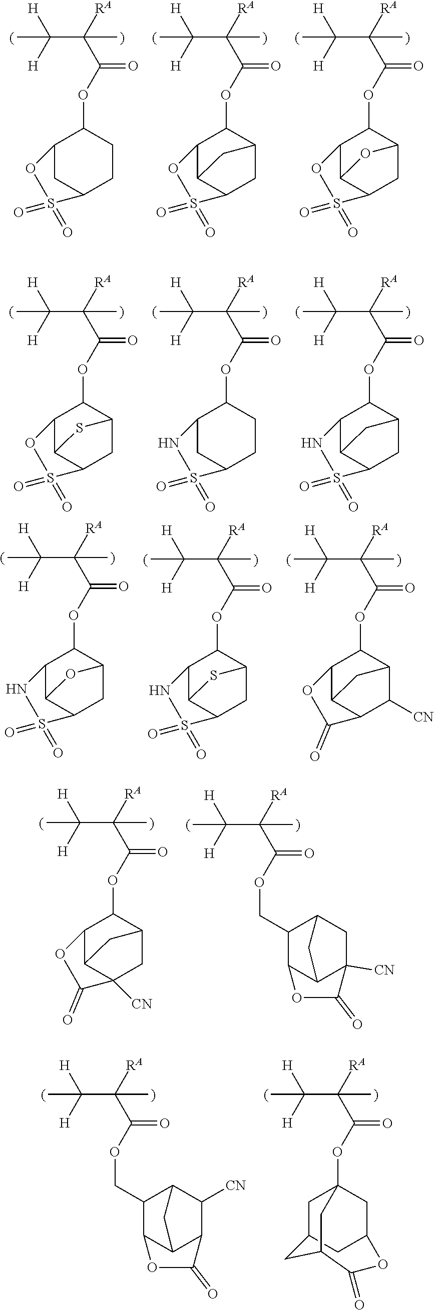

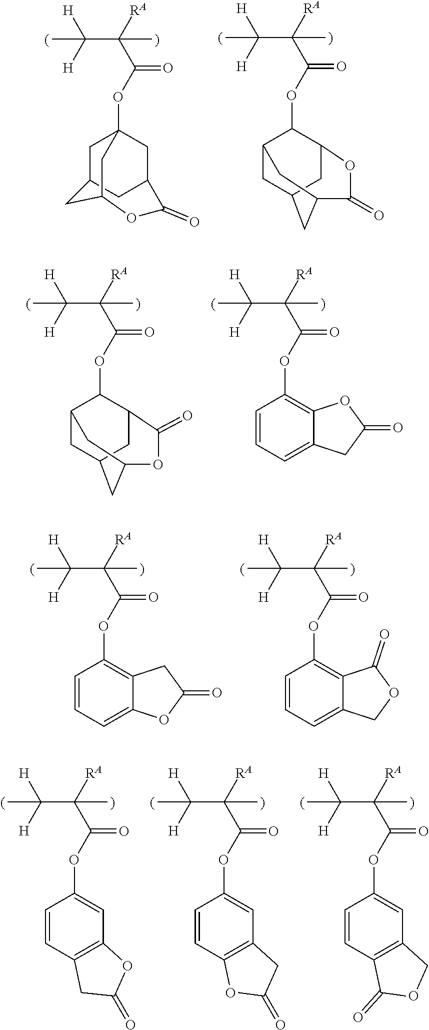

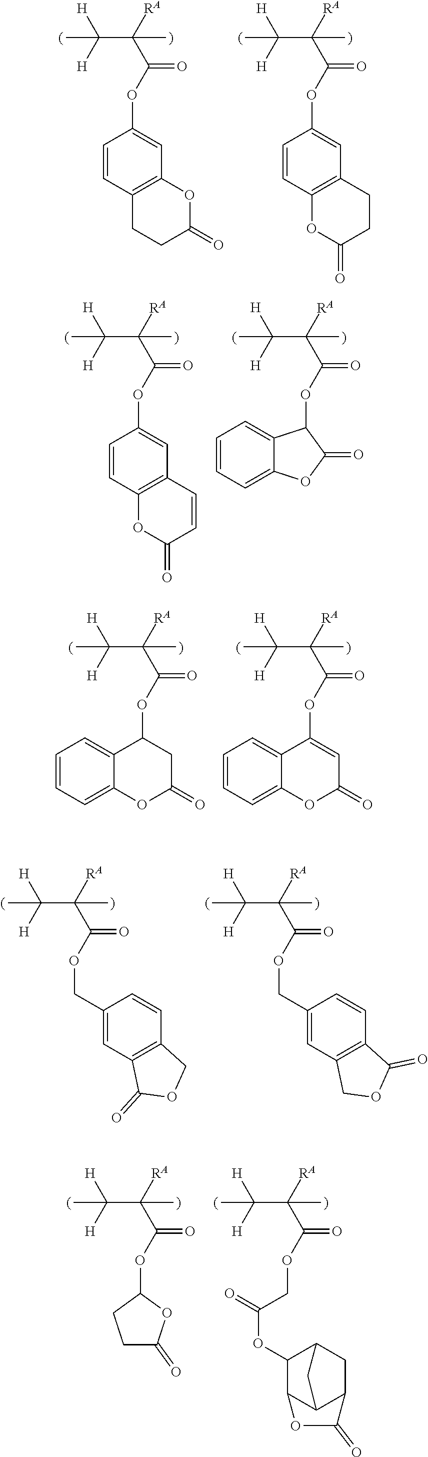

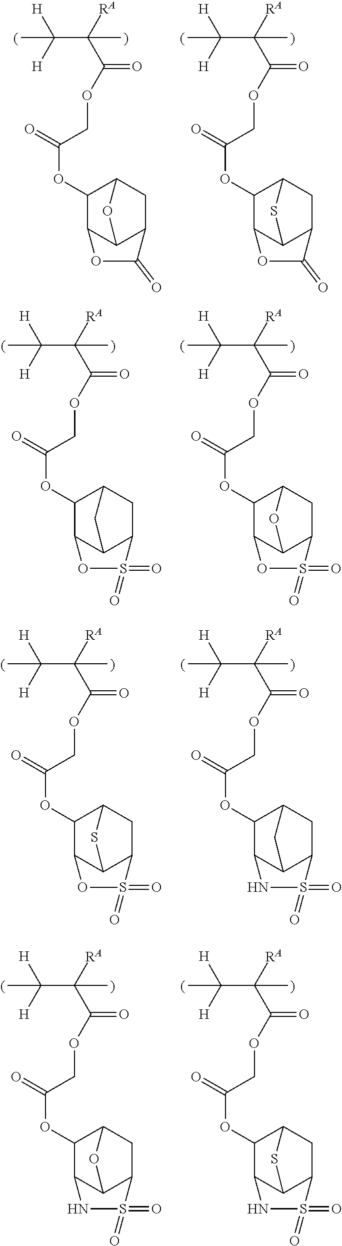

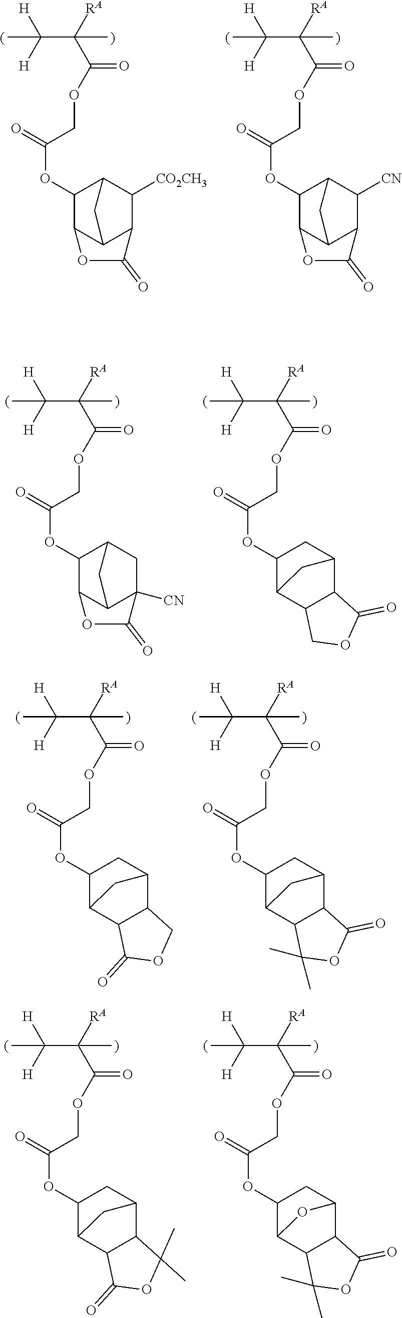

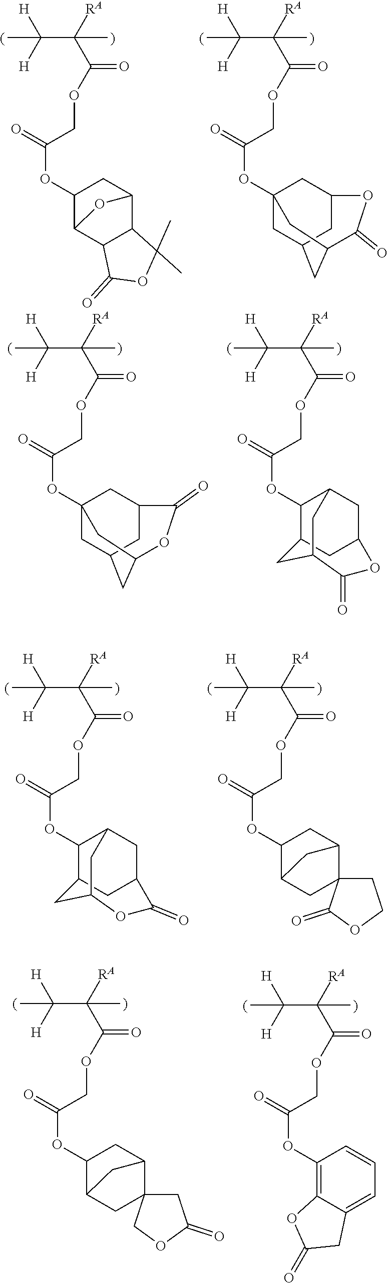

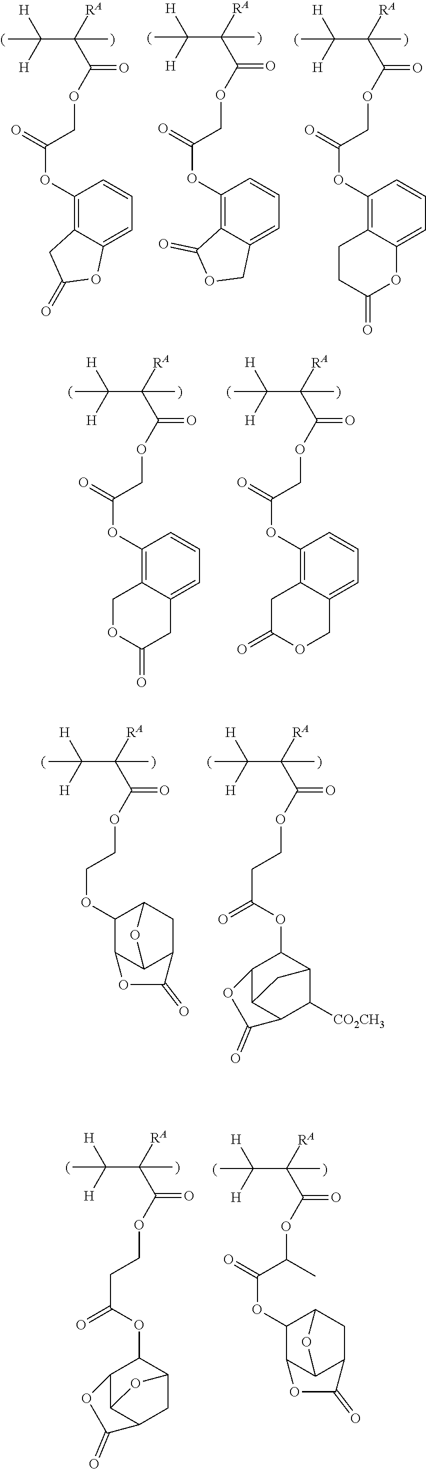

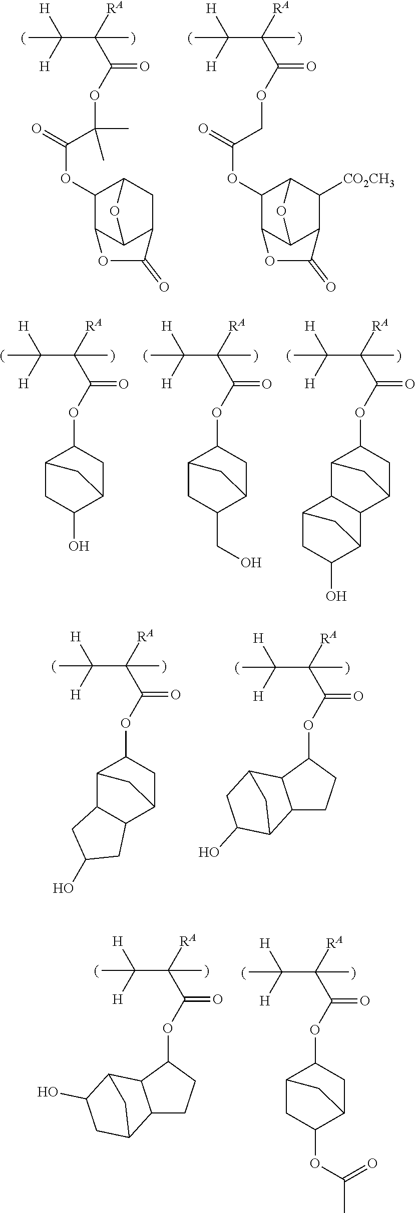

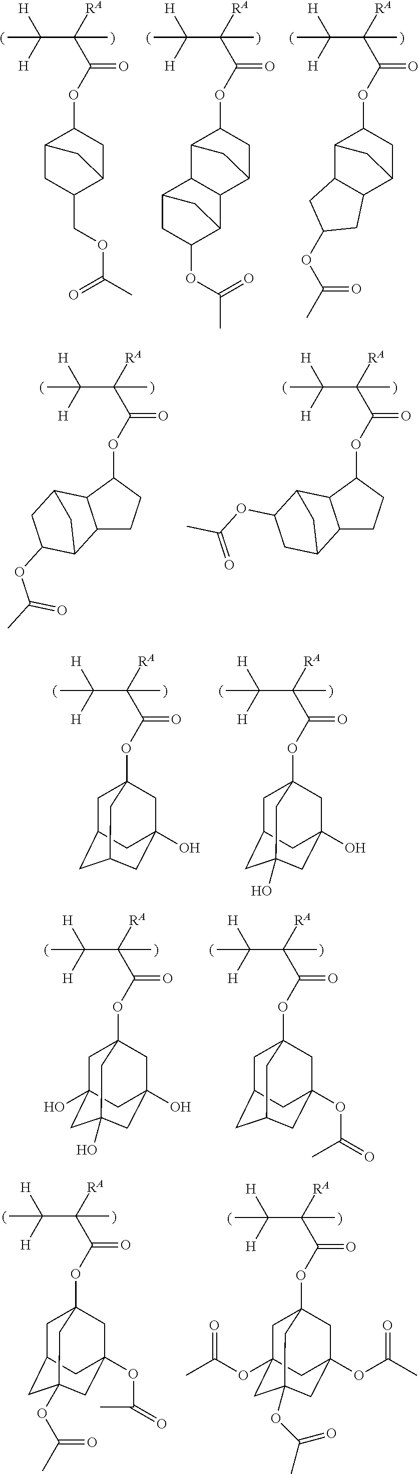

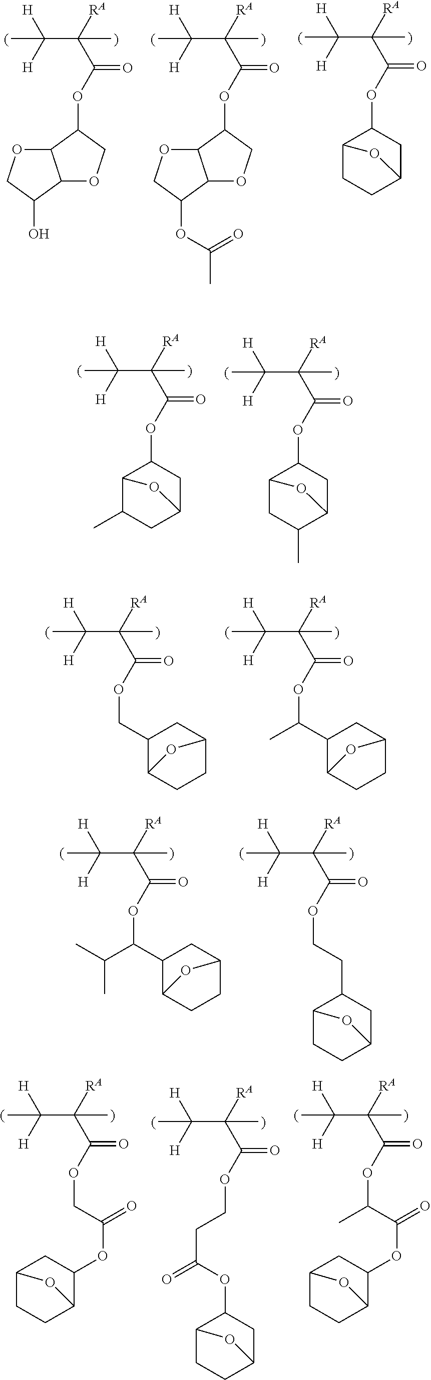

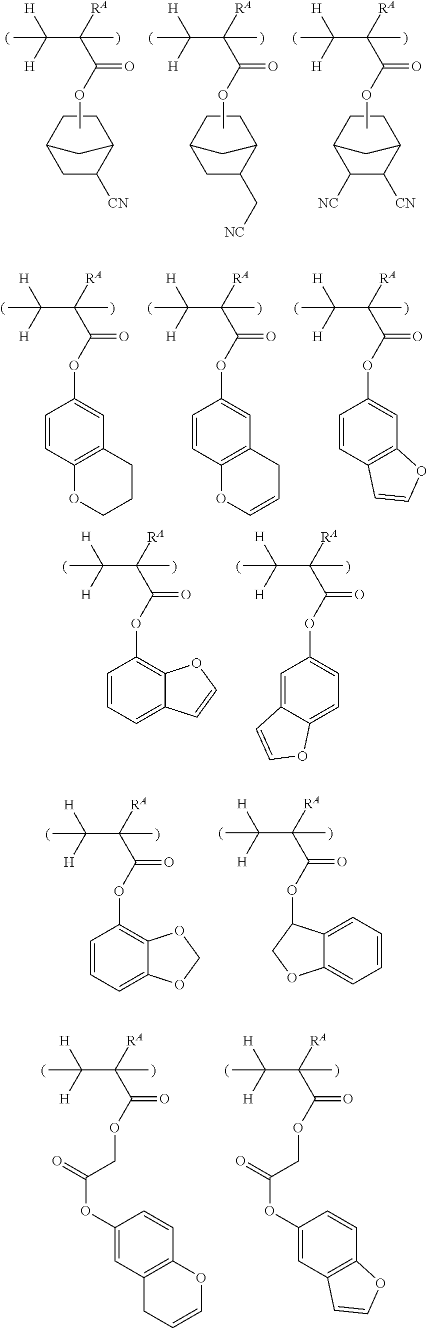

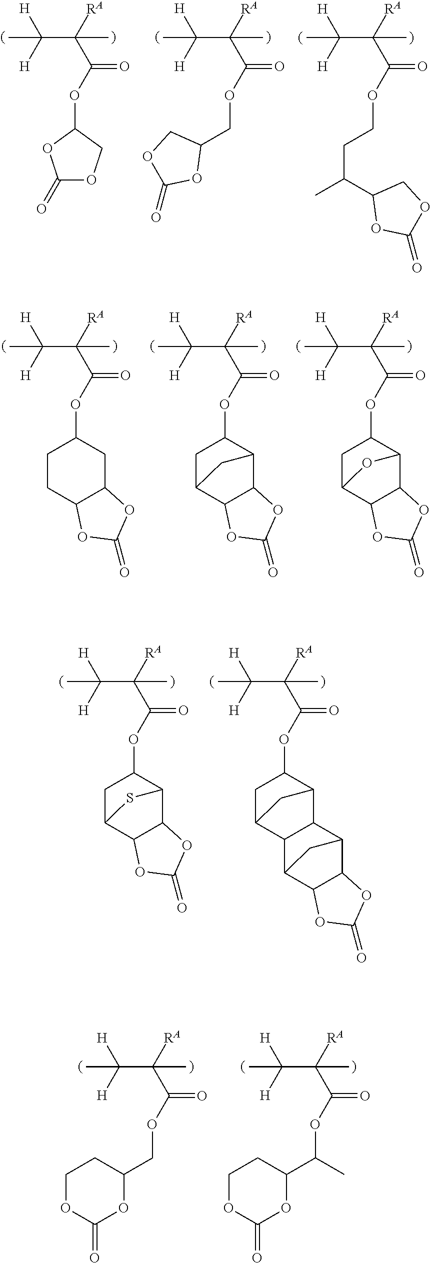

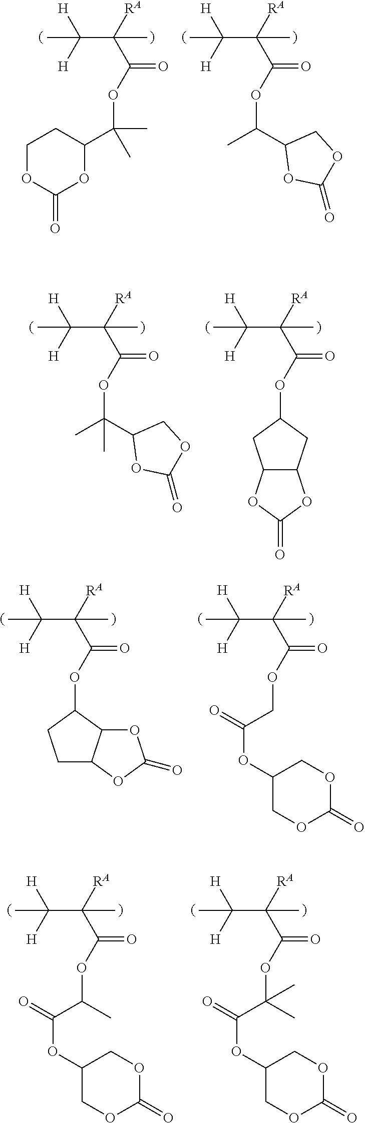

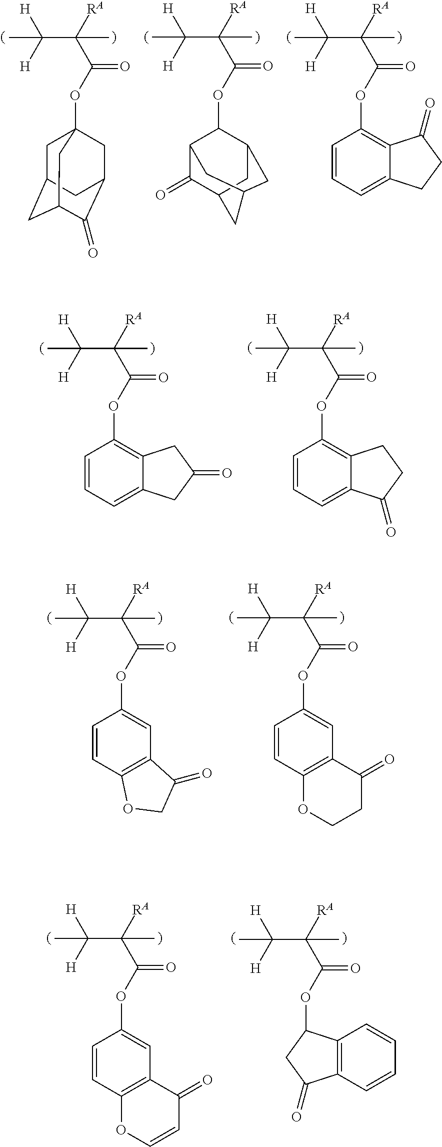

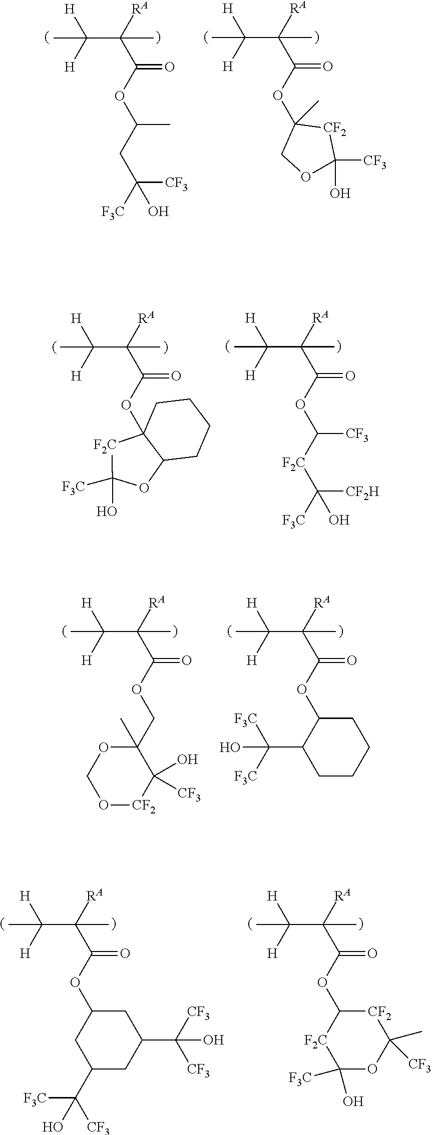

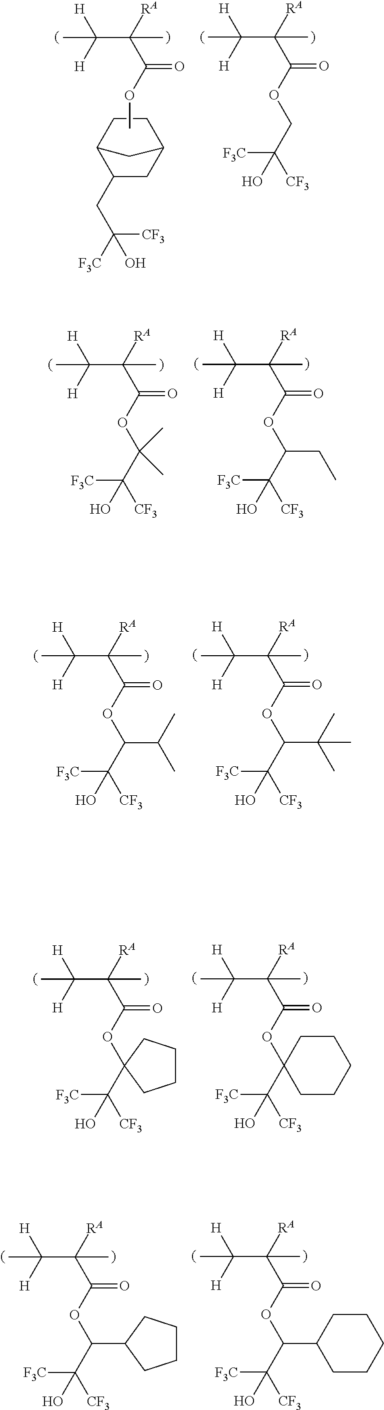

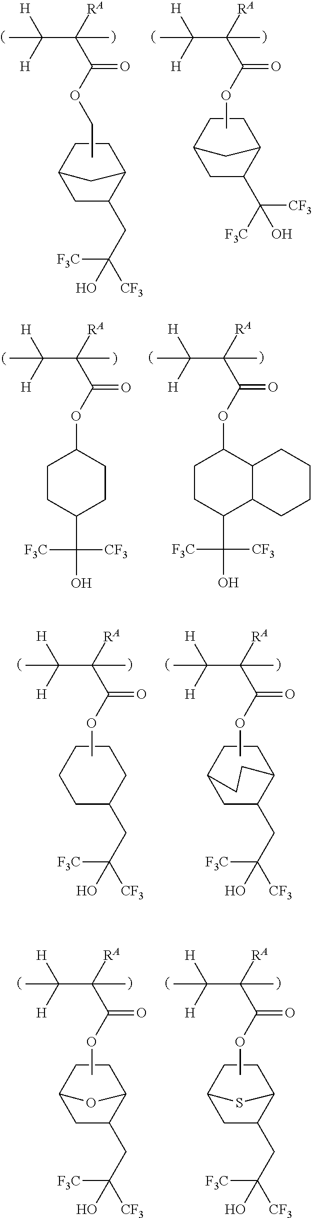

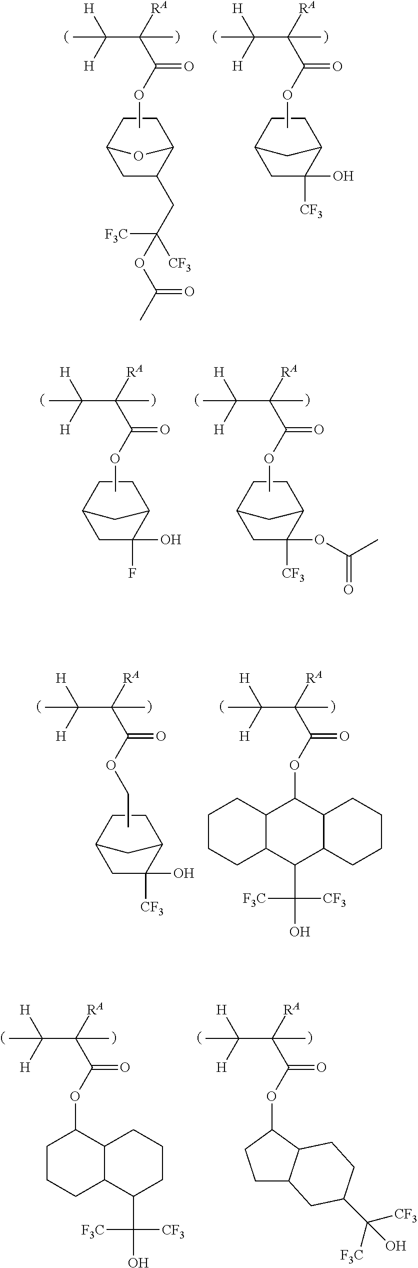

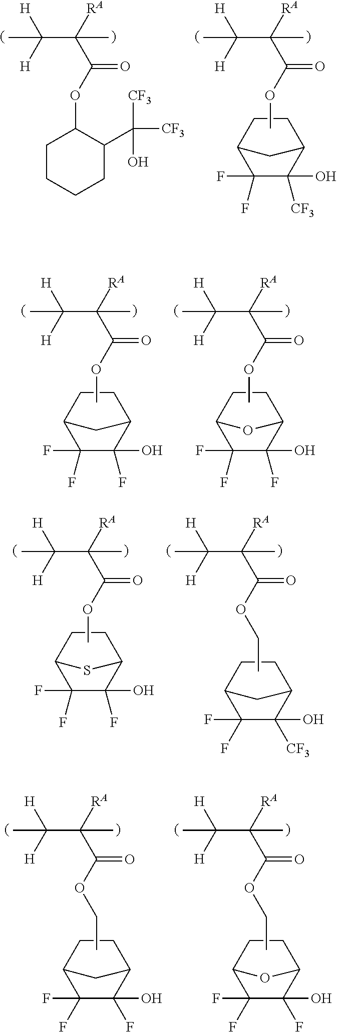

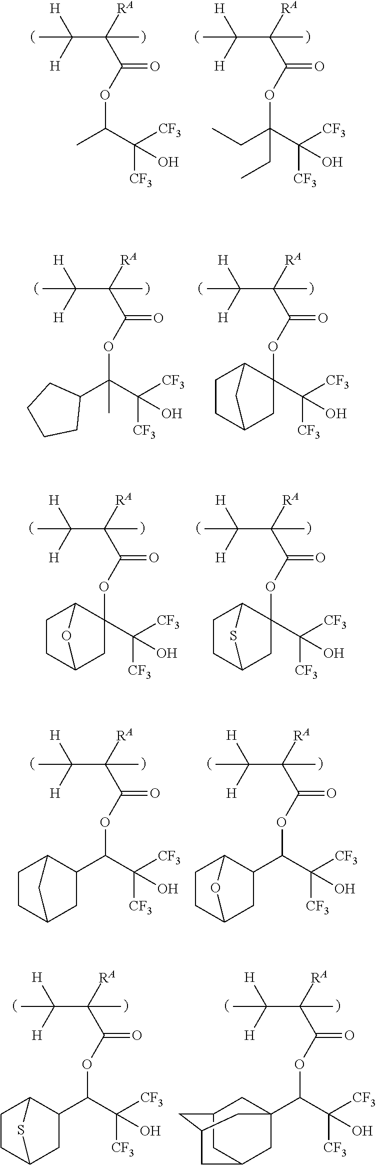

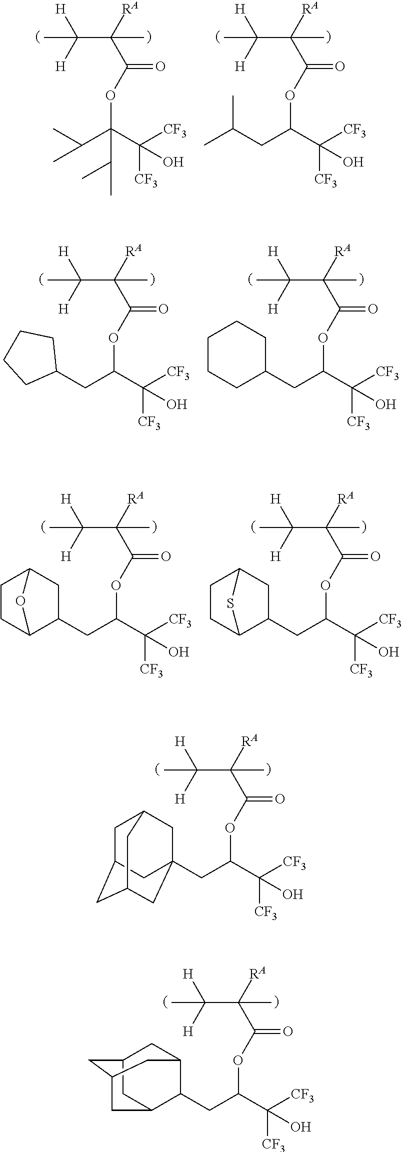

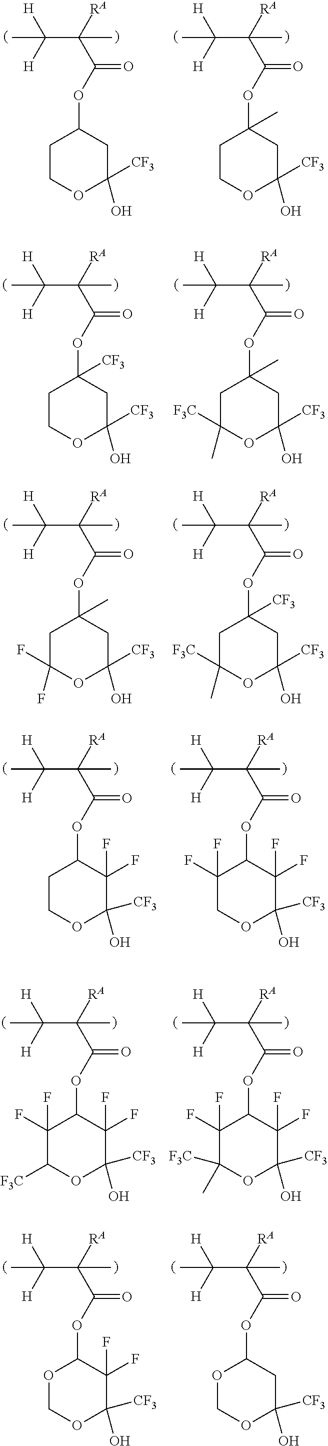

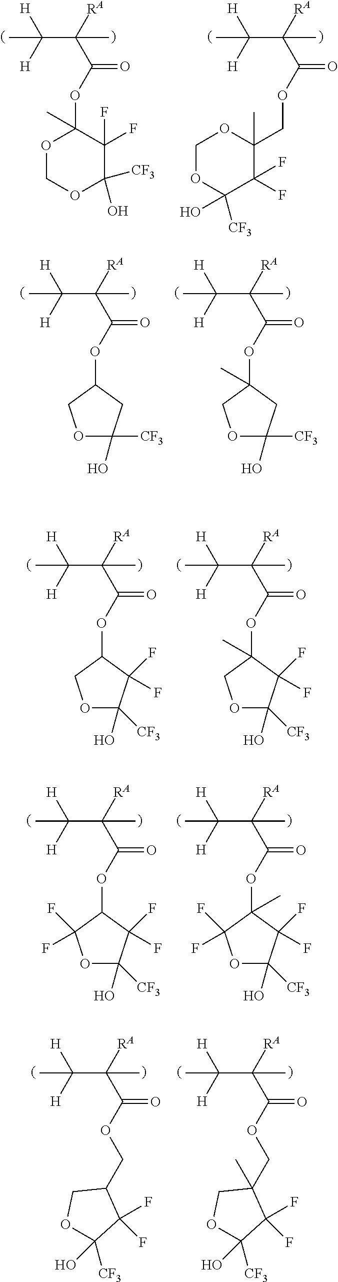

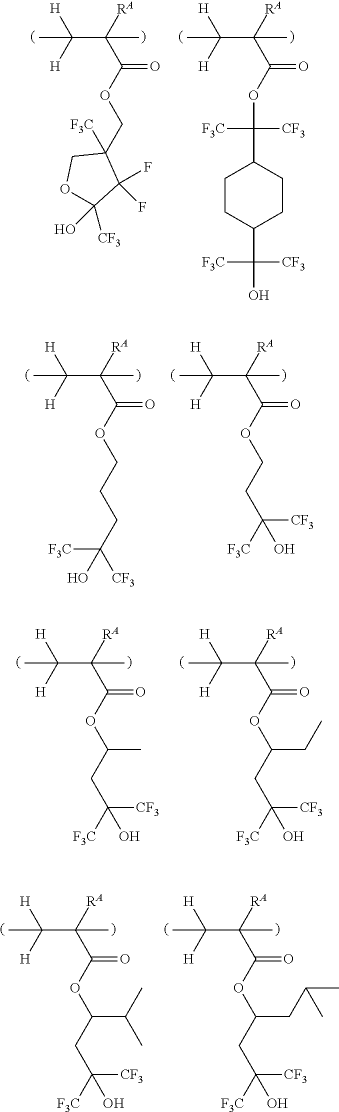

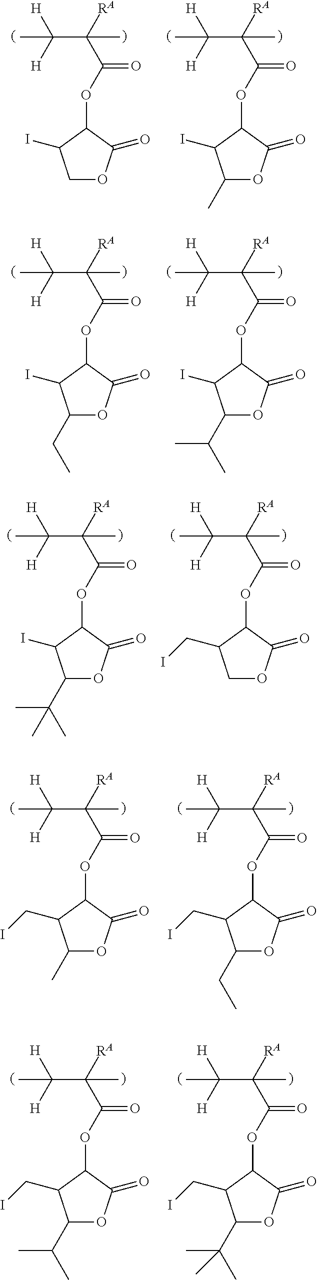

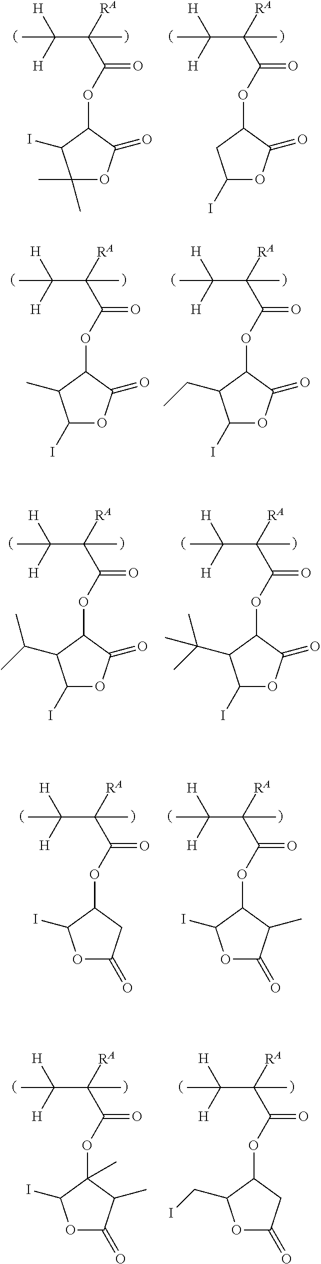

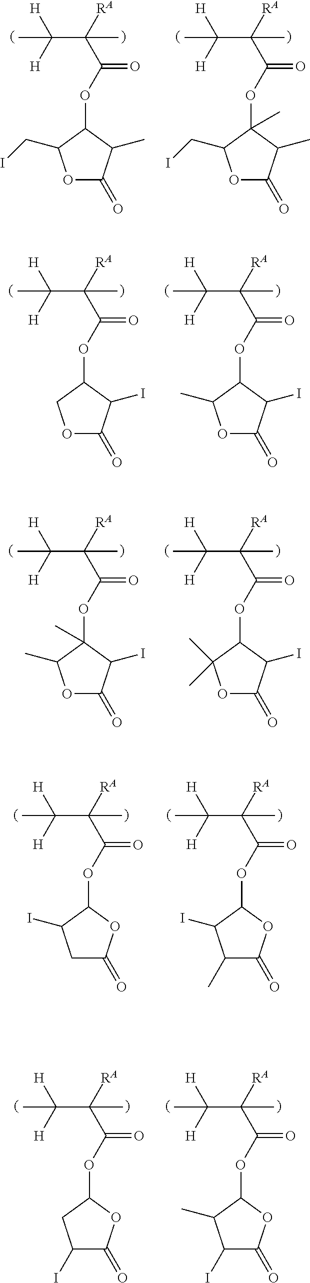

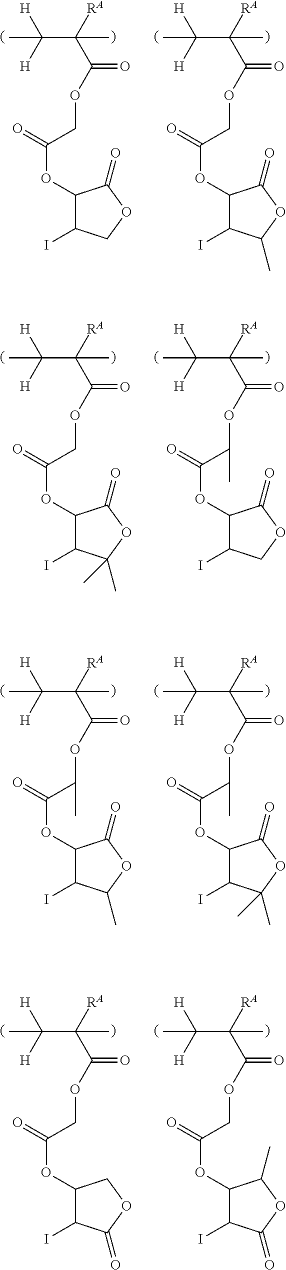

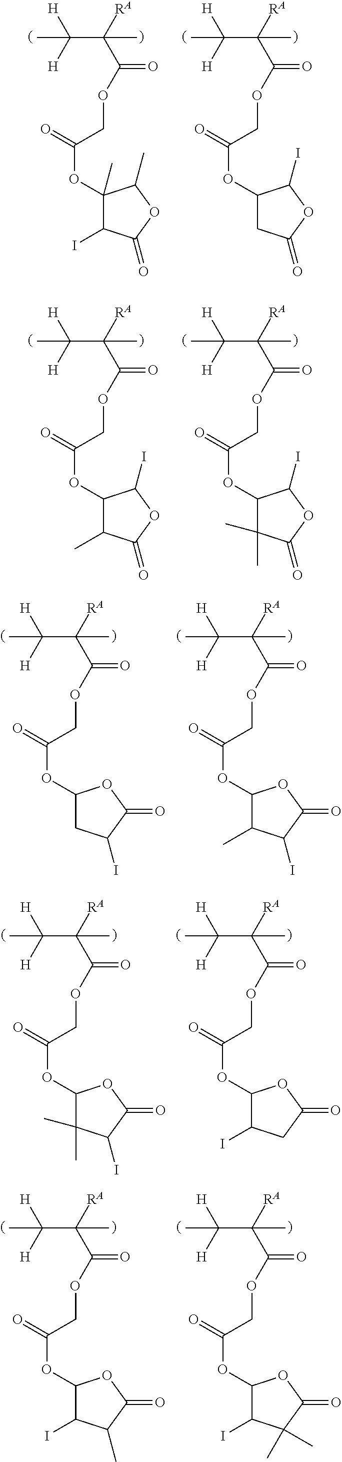

Specific examples of the repeating unit b2 are given below, but not limited thereto. In formulae below, R^{A} is the same as above.

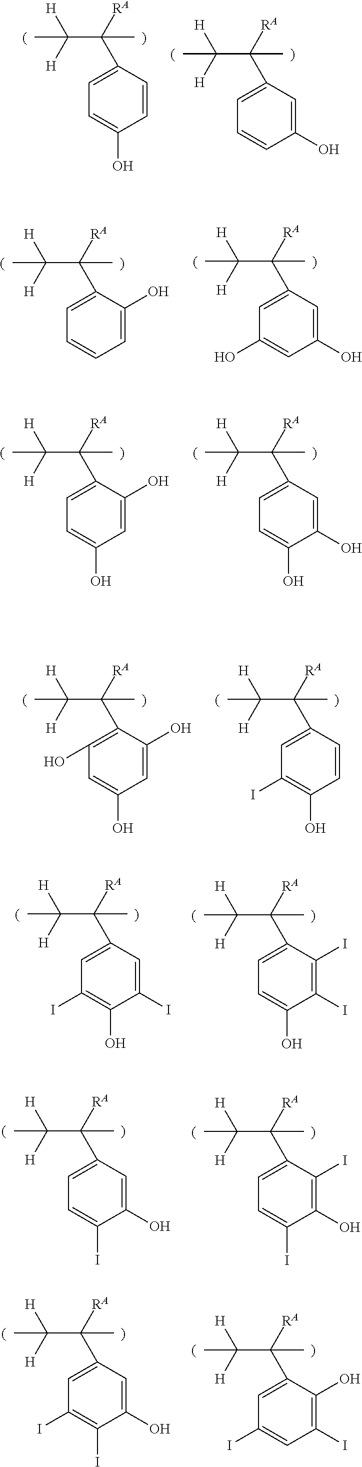

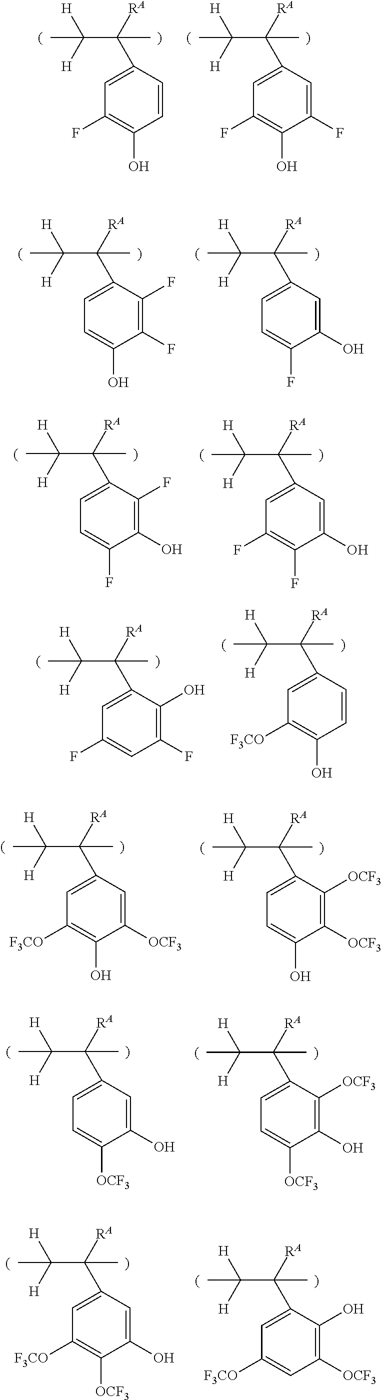

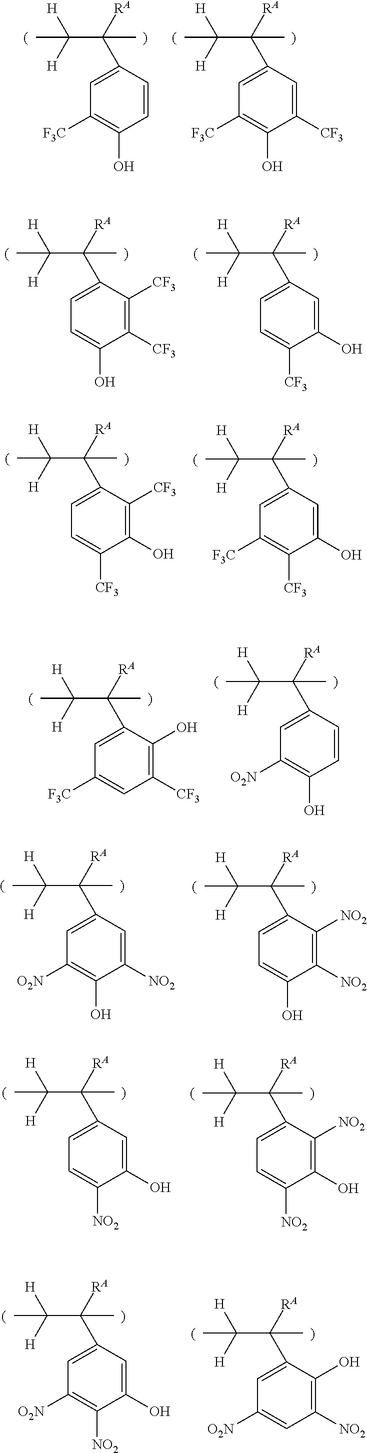

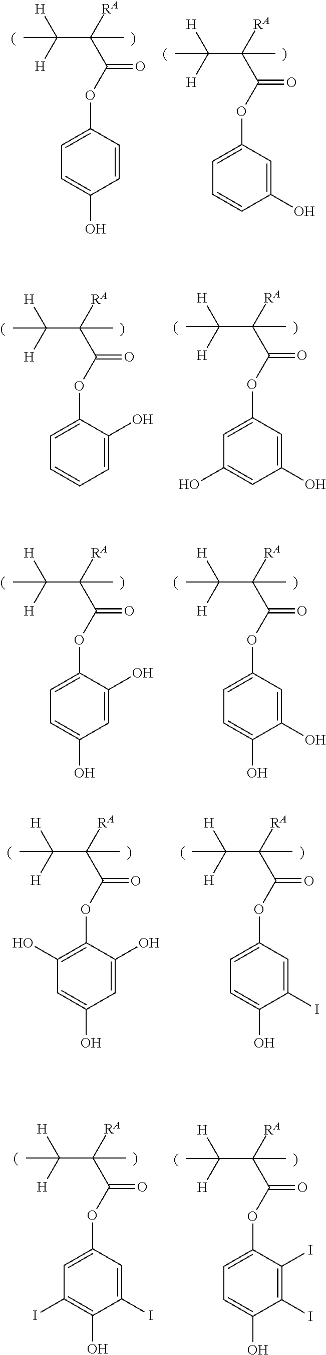

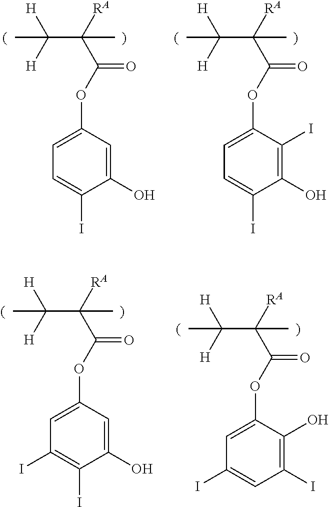

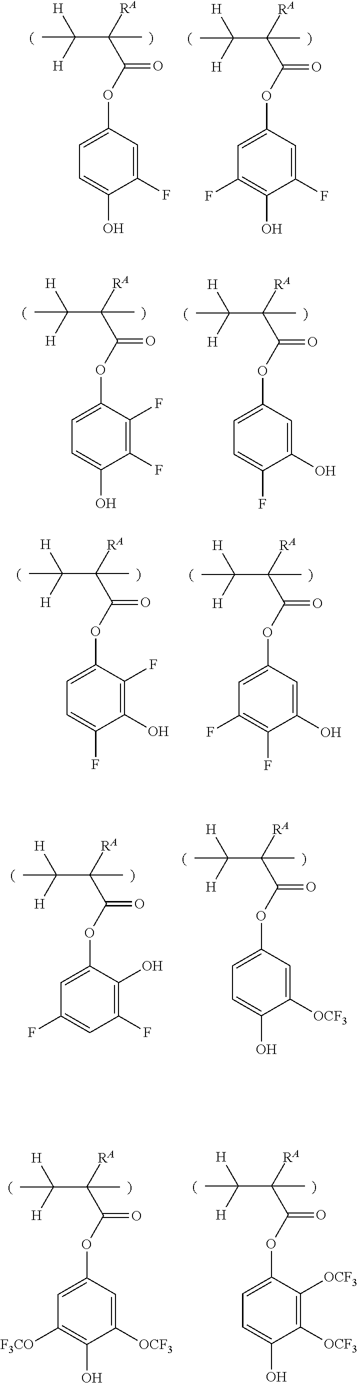

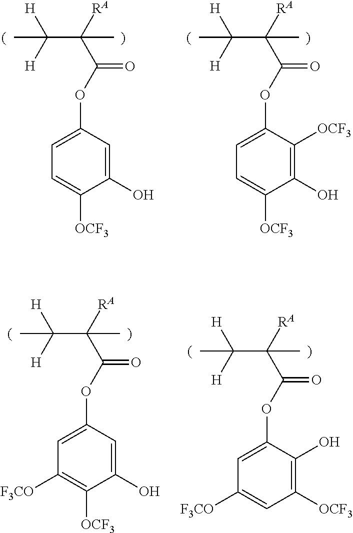

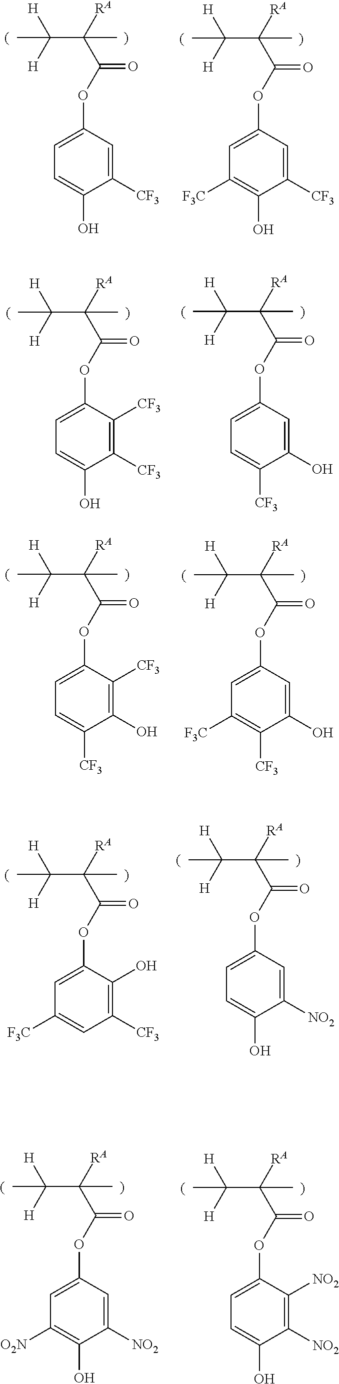

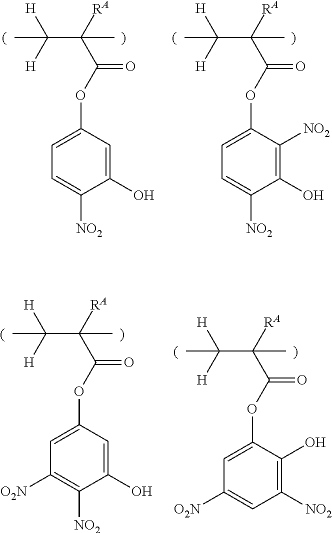

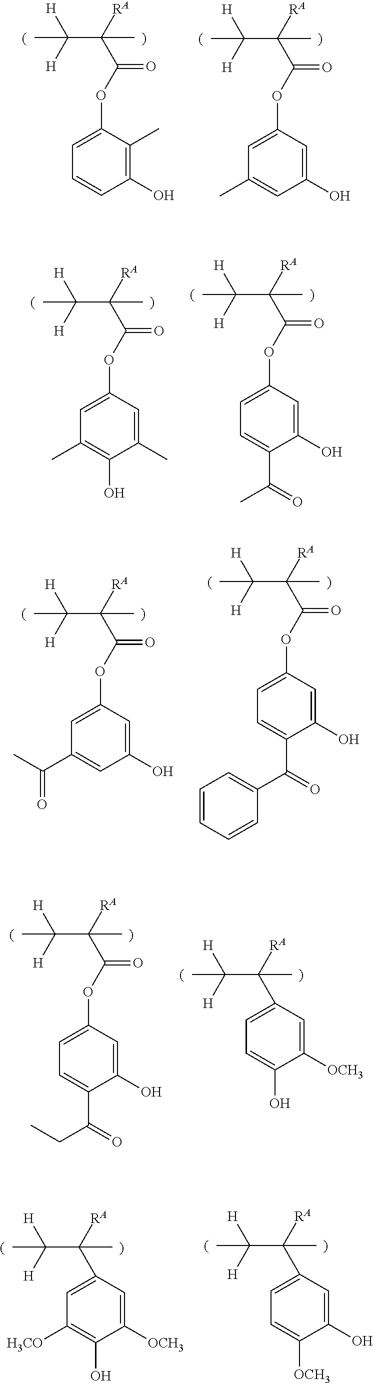

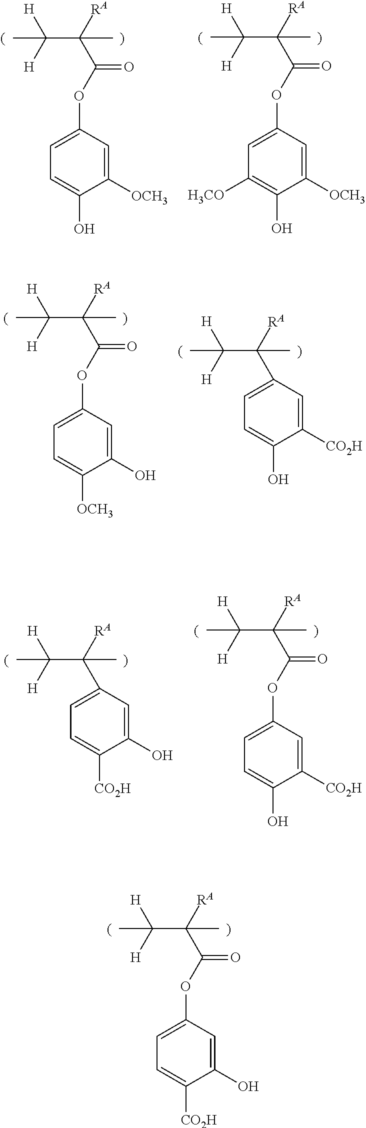

The repeating unit b1 or b2 preferably has a lactone ring as the polar group in the ArF lithography process and a phenol moiety in the KrF, EB and EUV lithography processes.

The base polymer may further contain at least one selected from the group consisting of a repeating unit represented by general formula (c1) (hereinafter referred to as repeating unit c1), a repeating unit represented by general formula (c2) (hereinafter referred to as repeating unit c2), a repeating unit represented by general formula (c3) (hereinafter referred to as repeating unit c3), a repeating unit represented by general formula (c4) (hereinafter referred to as repeating unit c4), and a repeating unit represented by general formula (c5) (hereinafter referred to as repeating unit c5).

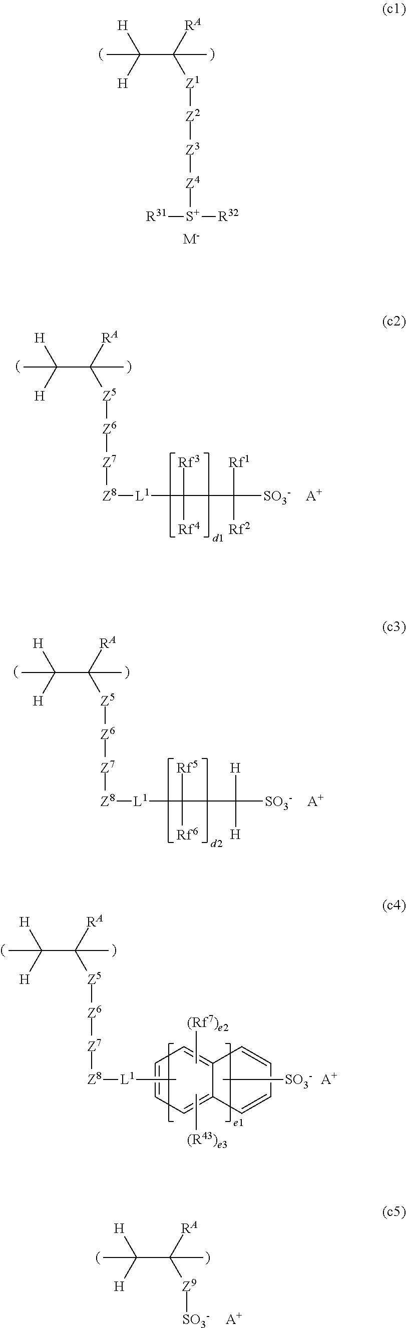

wherein “d1” and “d2” are each independently an integer of 0 to 3;
“e1” is 0 or 1, “e2” is an integer of 0 to 4, and “e3” is an integer of 0 to 4, provided that when “e1” is 0, 0≤e2+e3≤4, and when “e1” is 1, 0≤e2+e3≤6;
R^{A} is each independently a hydrogen atom, a fluorine atom, a methyl group, or a trifluoromethyl group;
Z¹ is a single bond or a phenylene group optionally having a substituent;
Z² is a single bond, **—C(═O)—O—Z²¹—, **—C(═O)—NH—Z²¹—, or **—O—Z²¹—, where Z²¹ is an aliphatic hydrocarbylene group having 1 to 6 carbon atoms, a phenylene group, or a divalent group obtained by combining these groups, and optionally comprises a halogen atom, a carbonyl group, an ester bond, an ether bond, or a hydroxy group;
Z³ is a single bond, an ether bond, an ester bond, a sulfonate ester bond, an amide bond, a sulfonamide bond, a carbonate bond, or a carbamate bond;
Z⁴ is a single bond, an aliphatic hydrocarbylene group having 1 to 6 carbon atoms, a phenylene group, or a divalent group obtained by combining these groups, and optionally comprises a halogen atom, a carbonyl group, an ester bond, an ether bond, or a hydroxy group;
Z⁵ is each independently a single bond, a phenylene group or naphthylene group optionally having a substituent, or *—C(═O)—O—Z⁵¹—, where Z⁵¹ is an aliphatic hydrocarbylene group having 1 to 10 carbon atoms, a phenylene group, or a naphthylene group, wherein the aliphatic hydrocarbylene group optionally comprises a halogen atom, a hydroxy group, an ether bond, an ester bond, or a lactone ring;
Z⁶ is a single bond, an ether bond, an ester bond, a sulfonate ester bond, an amide bond, a sulfonamide bond, a carbonate bond, or a carbamate bond;
each Z⁷ is independently a single bond, ***—Z⁷¹—C(═O)—O—, ***—C(═O)—NH—Z⁷¹—, or ***—O—Z⁷¹—, where Z⁷¹ is a hydrocarbylene group having 1 to 20 carbon atoms and optionally comprising a heteroatom;
each Z⁸ is independently a single bond, ****—Z⁸¹—C(═O)—O—, ****—C(═O)—NH—Z⁸¹—, or ****—O—Z⁸¹—, where Z⁸¹ is a hydrocarbylene group having 1 to 20 carbon atoms and optionally comprising a heteroatom;
Z⁹ is a single bond, a methylene group, an ethylene group, a phenylene group, a fluorinated phenylene group, a phenylene group substituted with a trifluoromethyl group, *—C(═O)—O—Z⁹¹—, *—C(═O)—NH—Z⁹¹—, or *—O—Z⁹¹—, where Z⁹¹ is an aliphatic hydrocarbylene group having 1 to 6 carbon atoms, a phenylene group, a fluorinated phenylene group, or a phenylene group substituted with a trifluoromethyl group, and optionally comprises a carbonyl group, an ester bond, an ether bond, or a hydroxy group;
“*” represents a bond to a carbon atom of a main chain, “**” represents a bond to Z¹, and “***” represents a bond to Z⁶, and “****” represents a bond to Z⁷;
L¹ is a single bond, an ether bond, an ester bond, a carbonyl group, a sulfonate ester bond, a sulfonamide bond, a carbonate bond, or a carbamate bond;
Rf¹ and Rf² are each independently a fluorine atom or a fluorinated saturated hydrocarbyl group having 1 to 6 carbon atoms;
Rf³ and Rf⁴ are each independently a hydrogen atom, a fluorine atom, or a fluorinated saturated hydrocarbyl group having 1 to 6 carbon atoms;
Rf⁵ and Rf⁶ are each independently a hydrogen atom, a fluorine atom, or a fluorinated saturated hydrocarbyl group having 1 to 6 carbon atoms, provided that not all Rf⁵ and Rf⁶ are hydrogen atoms at the same time;
RF⁷ is a fluorine atom, a fluorinated alkyl group having 1 to 6 carbon atoms, a fluorinated alkoxy group having 1 to 6 carbon atoms, or a fluorinated alkylthio group having 1 to 6 carbon atoms;
R⁴¹ and R⁴² are each independently a hydrocarbyl group having 1 to 20 carbon atoms and optionally comprising a heteroatom, or R⁴¹ and R⁴² are optionally bonded to each other to form a ring together with a sulfur atom to which the R⁴¹ and R⁴² are bonded;
R⁴³ is a halogen atom other than a fluorine atom or a hydrocarbyl group having 1 to 20 carbon atoms and optionally comprising a heteroatom, wherein when “e3” is 2, 3, or 4, a plurality of R⁴³ are optionally bonded to each other to form a ring together with carbon atoms to which the R⁴³ are bonded;
M⁻ is a non-nucleophilic counter ion; and
A⁺ is an onium cation.

In formulae(c1) to (c5), each R^{A} is independently a hydrogen atom, a fluorine atom, a methyl group, or a trifluoromethyl group; Z¹ is a single bond or a phenylene group optionally having a substituent; Z² is a single bond, **—C(═O)—O—Z²¹—, **—C(═O)—NH—Z²¹—, or **—O—Z²¹—, where Z²¹ is an aliphatic hydrocarbylene group having 1 to 6 carbon atoms, a phenylene group, or a divalent group obtained by combining these groups, and optionally comprises a halogen atom, a carbonyl group, an ester bond, an ether bond, or a hydroxy group; Z³ is a single bond, an ether bond, an ester bond, an amide bond, a sulfonate ester bond, a sulfonamide bond, a carbonate bond, or a carbamate bond; Z⁴ is a single bond, an aliphatic hydrocarbylene group having 1 to 6 carbon atoms, a phenylene group, or a divalent group obtained by combining these groups, and optionally comprises a halogen atom, a carbonyl group, an ester bond, an ether bond, or a hydroxy group; Z⁵ is each independently a single bond, a phenylene group or naphthylene group optionally having a substituent, or *—C(═O)—O—Z⁵¹—, where Z⁵¹ is an aliphatic hydrocarbylene group having 1 to 10 carbon atoms, a phenylene group, or a naphthylene group, wherein the aliphatic hydrocarbylene group optionally comprises a halogen atom, a hydroxy group, an ether bond, an ester bond, or a lactone ring; Z⁶ is a single bond, an ether bond, an ester bond, an amide bond, a sulfonate ester bond, a sulfonamide bond, a carbonate bond, or a carbamate bond; each Z⁷ is independently a single bond, ***—Z⁷¹—C(═O)—O—, ***—C(═O)—NH—Z⁷¹—, or ***—O—Z⁷¹—, where Z⁷¹ is a hydrocarbylene group having 1 to 20 carbon atoms and optionally comprising a heteroatom;
each Z⁸ is independently a single bond, ****—Z^{B1}—C(═O)—O—, ****—C(═O)—NH—Z⁸¹—, or ****—O—Z⁸¹—, where Z⁸¹ is a hydrocarbylene group having 1 to 20 carbon atoms and optionally comprising a heteroatom; Z⁹ is a single bond, a methylene group, an ethylene group, a phenylene group, a fluorinated phenylene group, a phenylene group substituted with a trifluoromethyl group, *—C(═O)—O—Z⁹¹—, *—C(═O)—NH—Z⁹¹—, or *—O—Z⁹¹—, where Z⁹¹ is an aliphatic hydrocarbylene group having 1 to 6 carbon atoms, a phenylene group, a fluorinated phenylene group, or a phenylene group substituted with a trifluoromethyl group, and optionally comprises a carbonyl group, an ester bond, an ether bond, or a hydroxy group; “*” represents a bond to a carbon atom of a main chain, “**” represents a bond to Z¹, and “***” represents a bond to Z⁶, and “****” represents a bond to Z⁷;

The aliphatic hydrocarbylene groups represented by Z²¹, Z⁵¹, and Z⁹¹ may be linear, branched, or cyclic. Specific examples thereof include alkanediyl groups such as a methanediyl group, an ethane-1,1-diyl group, an ethane-1,2-diyl group, a propane-1,1-diyl group, a propane-1,2-diyl group, a propane-1,3-diyl group, a propane-2,2-diyl group, a butane-1,1-diyl group, a butane-1,2-diyl group, a butane-1,3-diyl group, a butane-2,3-diyl group, a butane-1,4-diyl group, a 1,1-dimethylethane-1,2-diyl group, a pentane-1,5-diyl group, a 2-methylbutane-1,2-diyl group, and a hexane-1,6-diyl group; cyctoalkanediyl groups such as a cyclopropanediyl group, a cyclobutanediyl group, a cyclopentanediyl group, and a cyclohexanediyl group; and groups obtained by combining these groups.

The hydrocarbylene groups optionally comprising a heteroatom, represented by Z⁷¹ and Z⁸¹, may be either saturated or unsaturated and linear, branched, or cyclic. Specific examples thereof are given below, but not limited thereto:

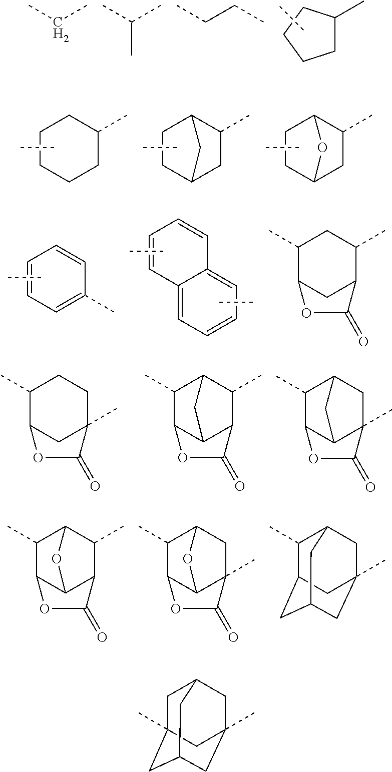

wherein the dashed line represents a bond.

In formula (c1), R⁴¹ and R⁴² are each independently a hydrocarbyl group having 1 to 20 carbon atoms and optionally comprising a heteroatom. The hydrocarbyl group may be either saturated or unsaturated and may be linear, branched, or cyclic. Specific examples thereof include alkyl groups having 1 to 20 carbon atoms, such as a methyl group, an ethyl group, an n-propyl group, an isopropyl group, an n-butyl group, an isobutyl group, a sec-butyl group, and a tert-butyl group; cyclic saturated hydrocarbyl groups having 3 to 20 carbon atoms, such as a cyclopropyl group, a cyclopentyl group, a cyclohexyl group, a cyclopropylmethyl group, a 4-methylcyclohexyl group, a cyclohexylmethyl group, a norbornyl group, and an adamantyl group; alkenyl groups having 2 to 20 carbon atoms, such as a vinyl group, a 1-propenyl group, a 2-propenyl group, a butenyl group, and a hexenyl group; cyclic unsaturated hydrocarbyl groups having 3 to 20 carbon atoms, such as a cyclohexenyl group; aryl groups having 6 to 20 carbon atoms, such as a phenyl group, a naphthyl group, and a thienyl group; aralkyl groups having 7 to 20 carbon atoms, such as a benzyl group, a 1-phenylethyl group, and a 2-phenylethyl group; and groups obtained by combining these groups, but the aryl group is preferable. Some or all of the hydrogen atoms in the hydrocarbyl group are optionally substituted with a group containing a heteroatom such as an oxygen atom, a sulfur atom, a nitrogen atom, or a halogen atom, and some of —CH₂— in the hydrocarbyl group are optionally substituted with a group containing a heteroatom such as an oxygen atom, a sulfur atom, or a nitrogen atom. As a result, the hydrocarbyl group may contain, for instance, a hydroxy group, a fluorine atom, a chlorine atom, a bromine atom, an iodine atom, a cyano group, a carbonyl group, an ether bond, an ester bond, a sulfonate ester bond, a carbonate bond, a lactone ring, a sultone ring, a carboxylic anhydride (—C(═O)—O—C(═O)—), and a haloalkyl group.

In addition, R⁴¹ and R⁴² are optionally bonded to each other to form a ring together with sulfur atoms to which the R⁴¹ and R⁴² are bonded. At this point, specific examples of the ring include those represented by formulae below:

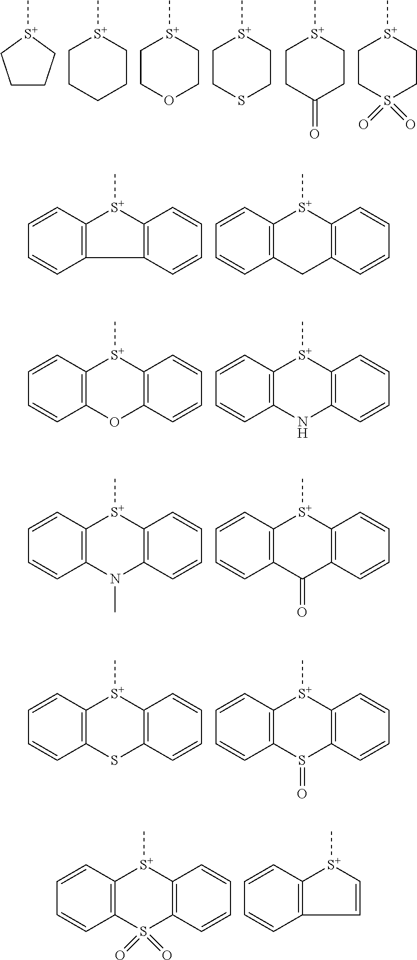

wherein the dashed line represents a bond to Z⁴.

Specific examples of the cation in the repeating unit c1 are given below, but not limited thereto. In formulae below, R^{A} is the same as above.

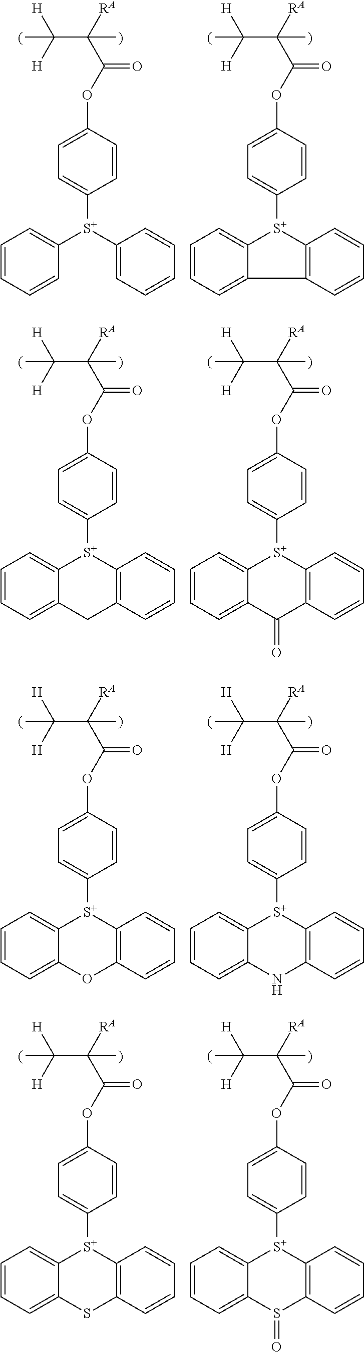

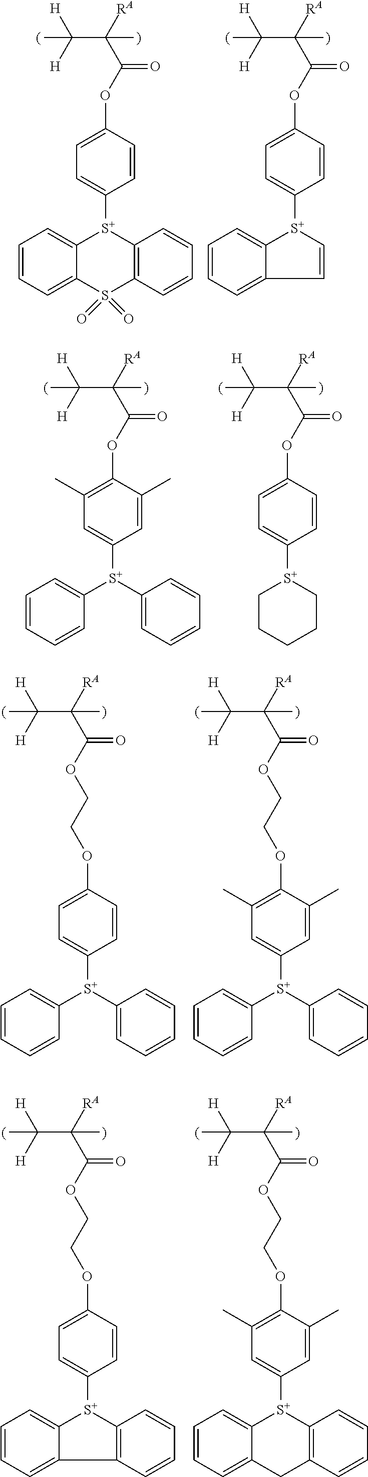

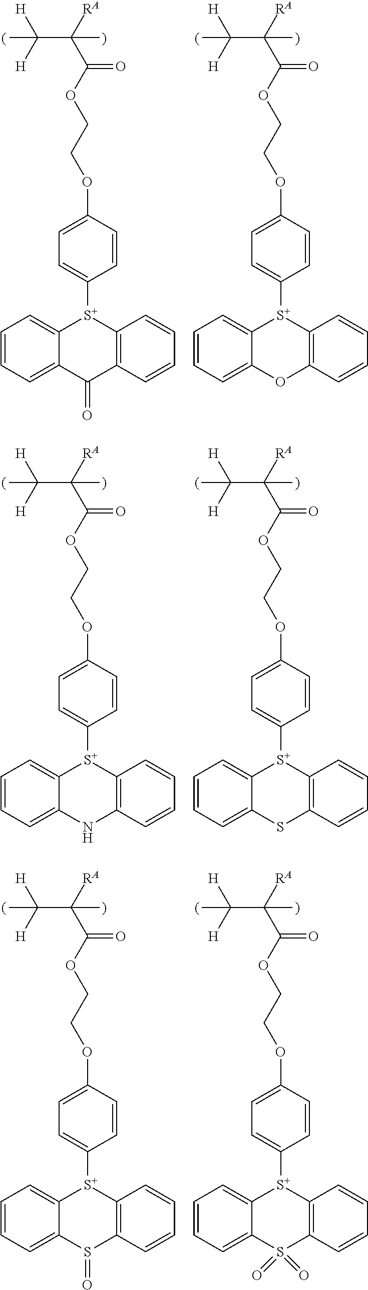

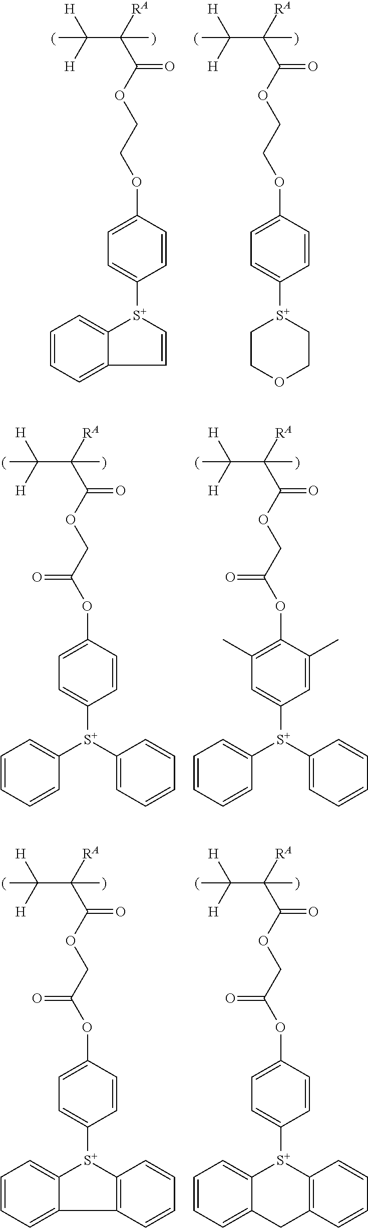

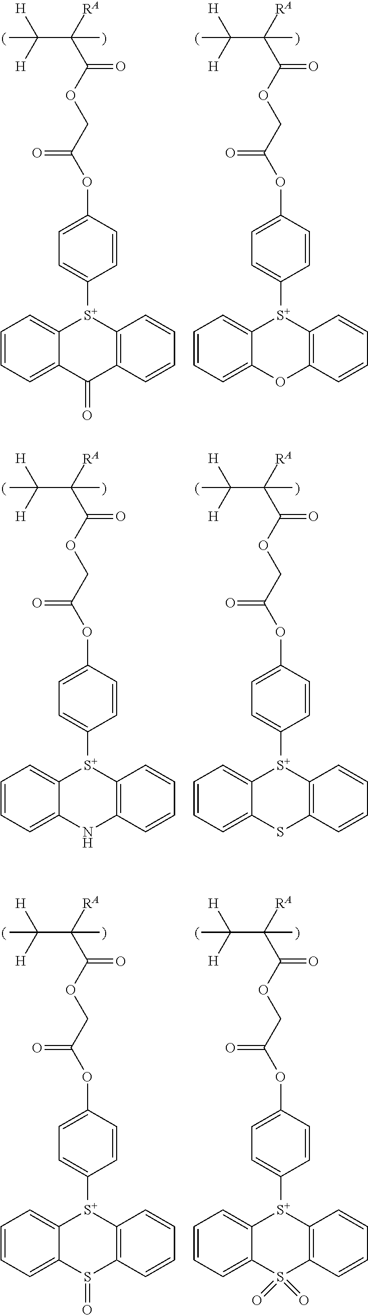

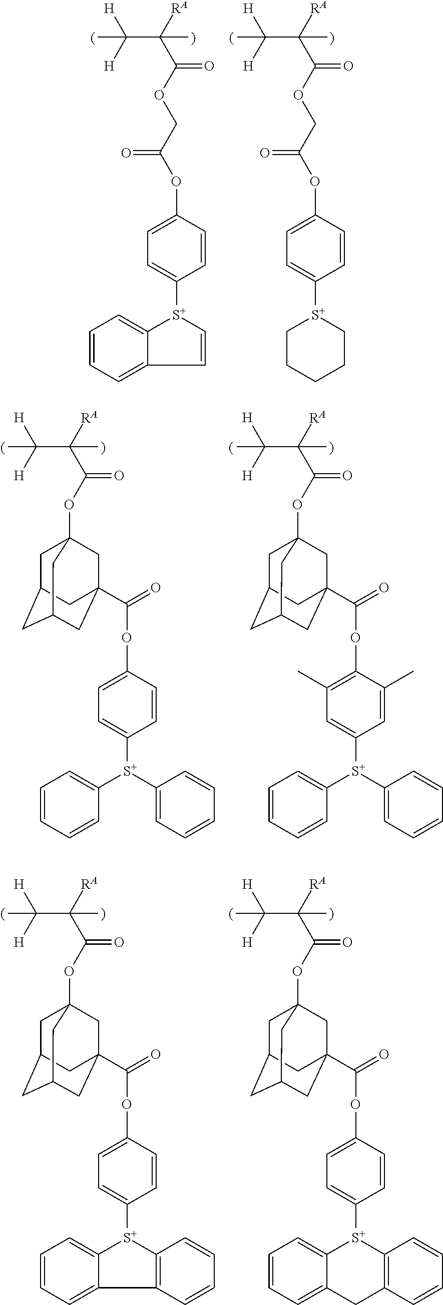

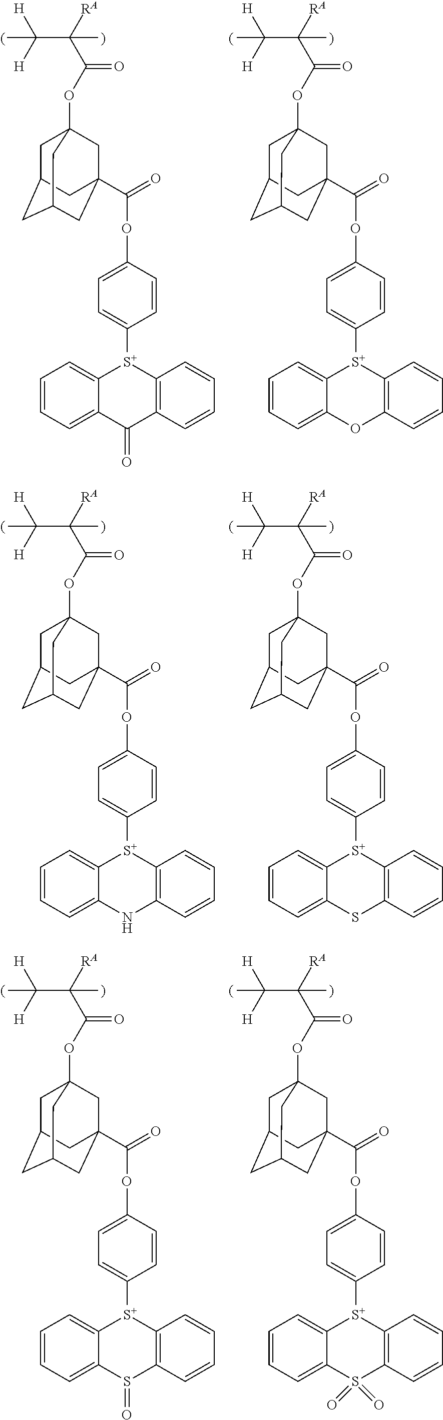

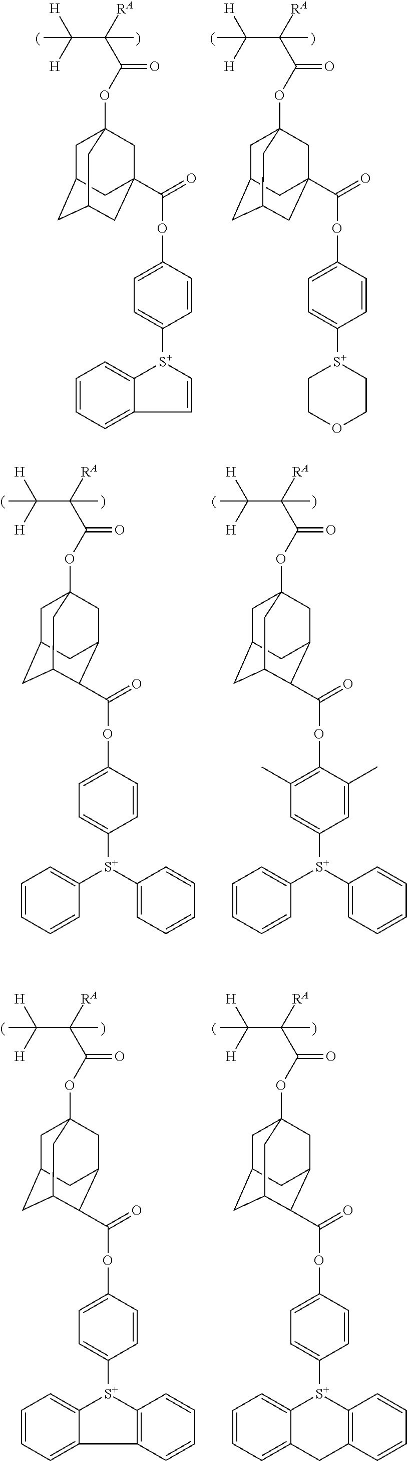

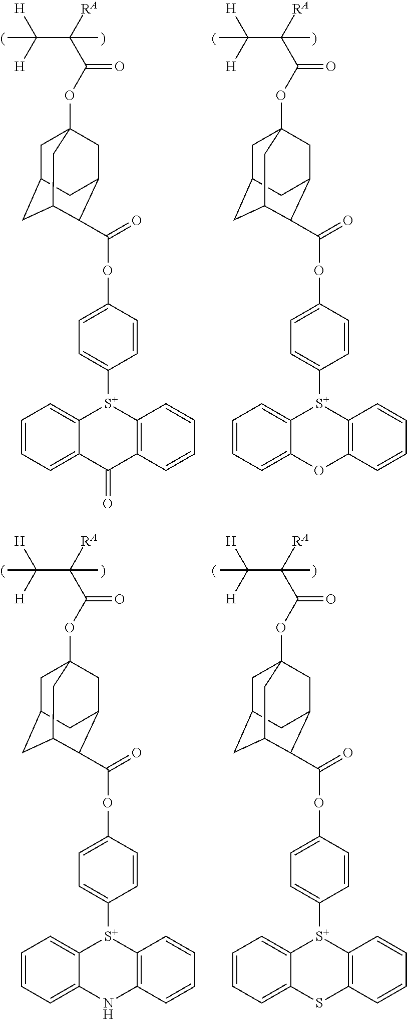

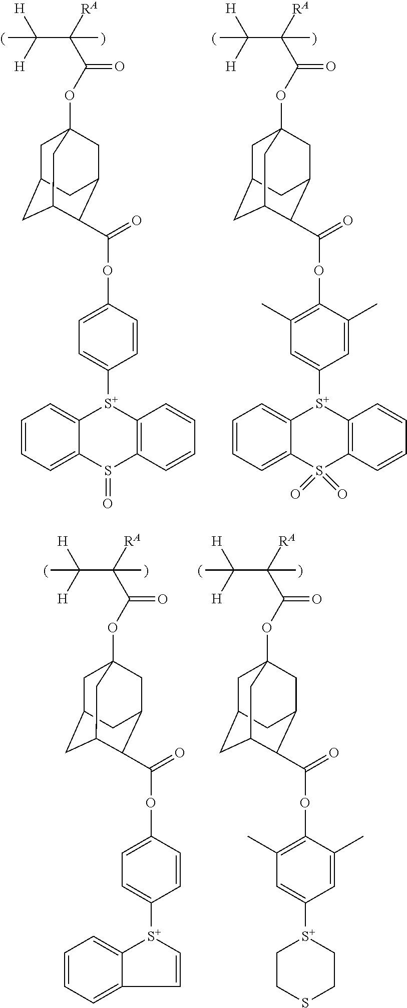

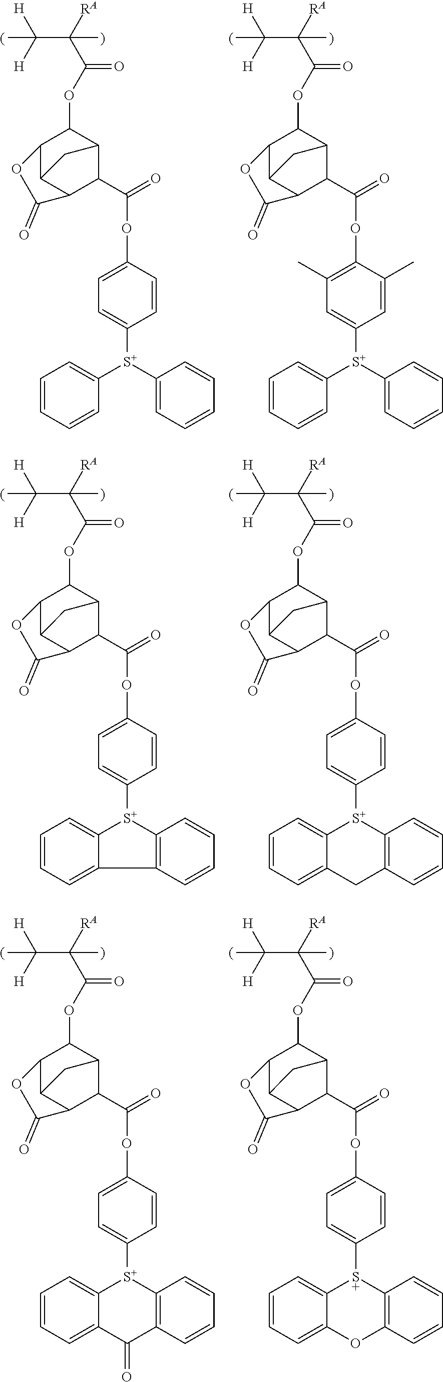

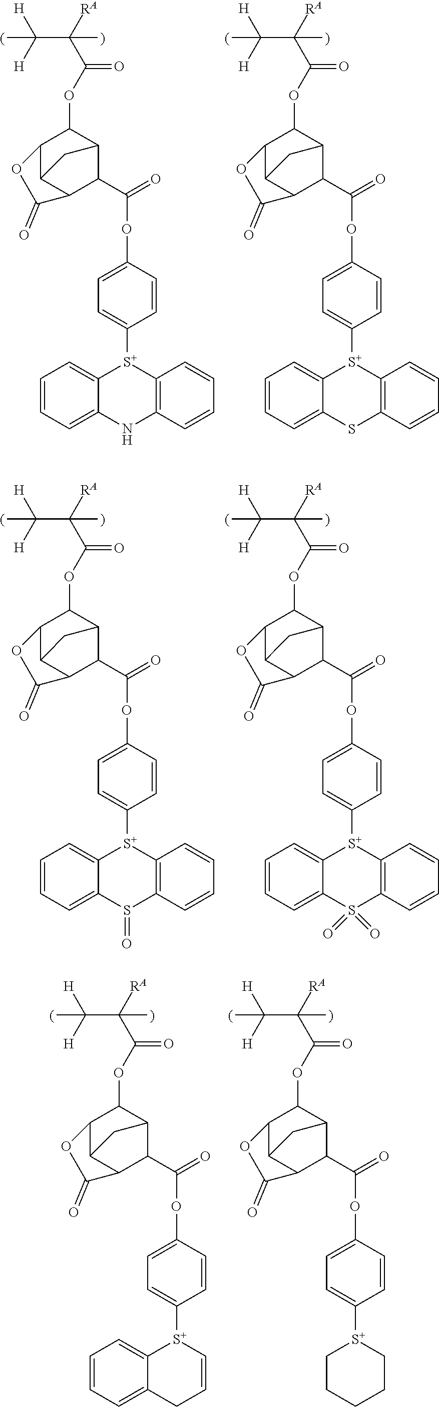

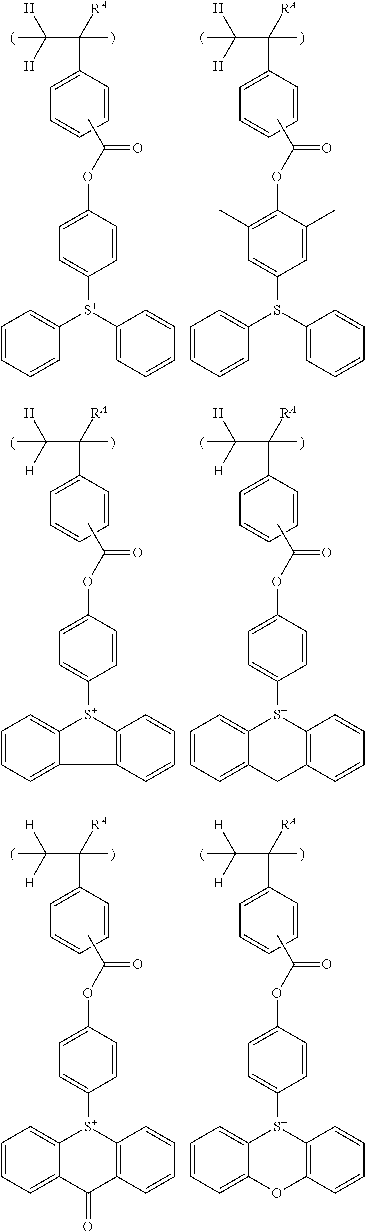

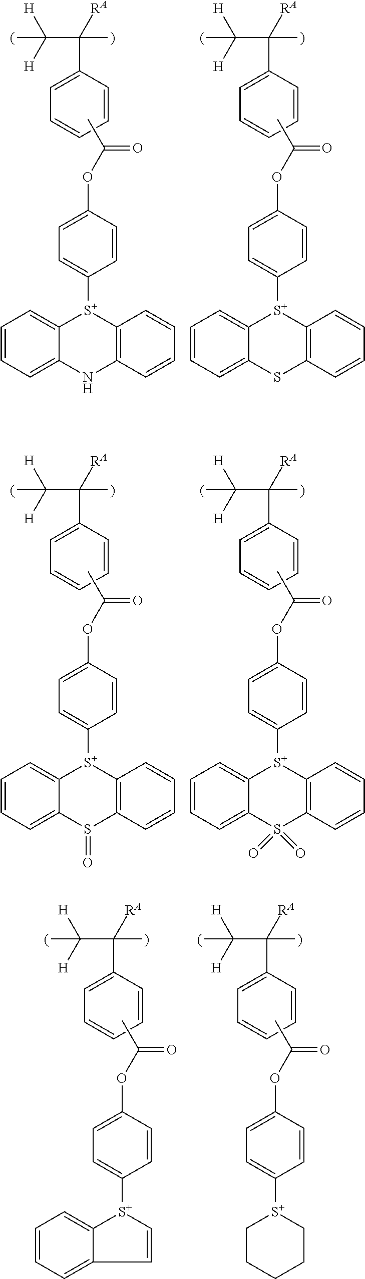

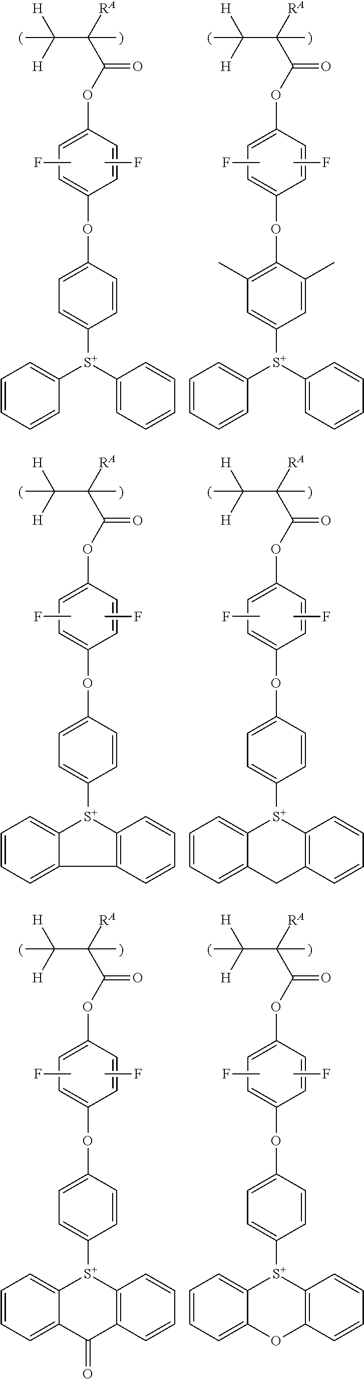

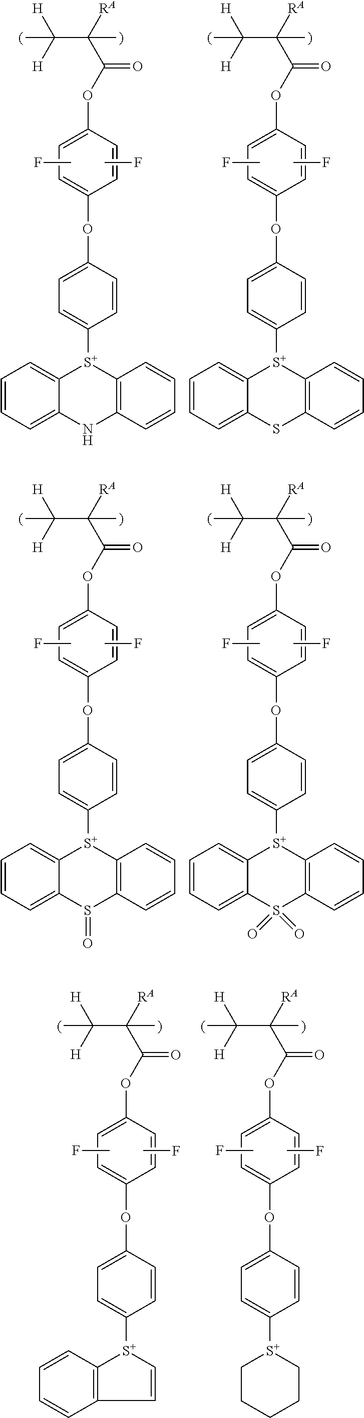

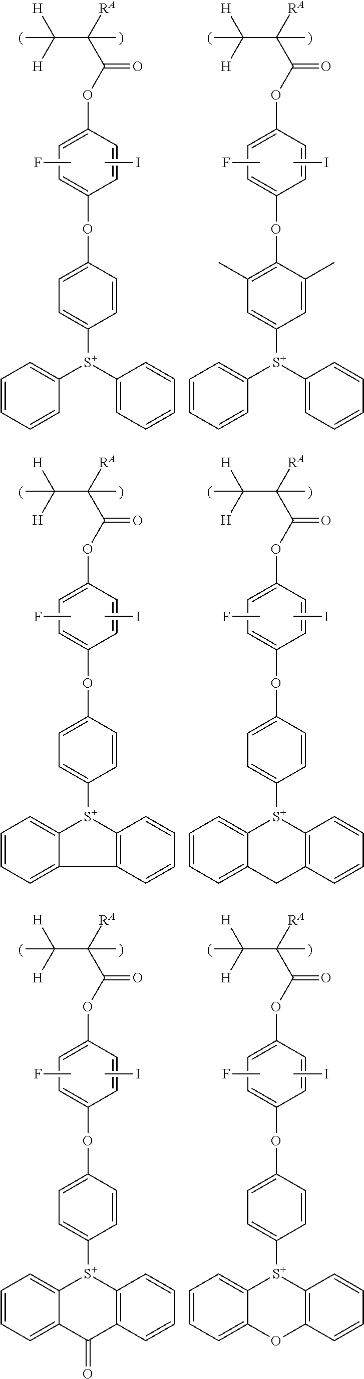

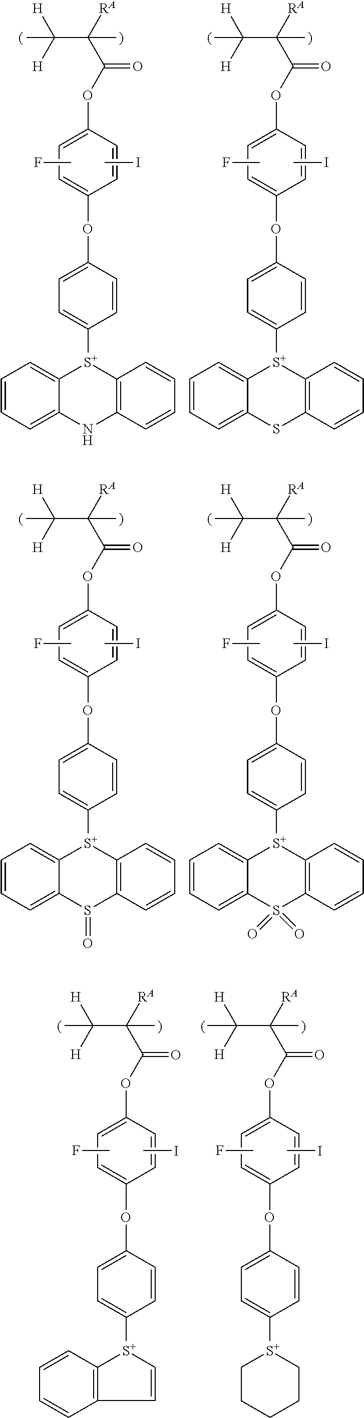

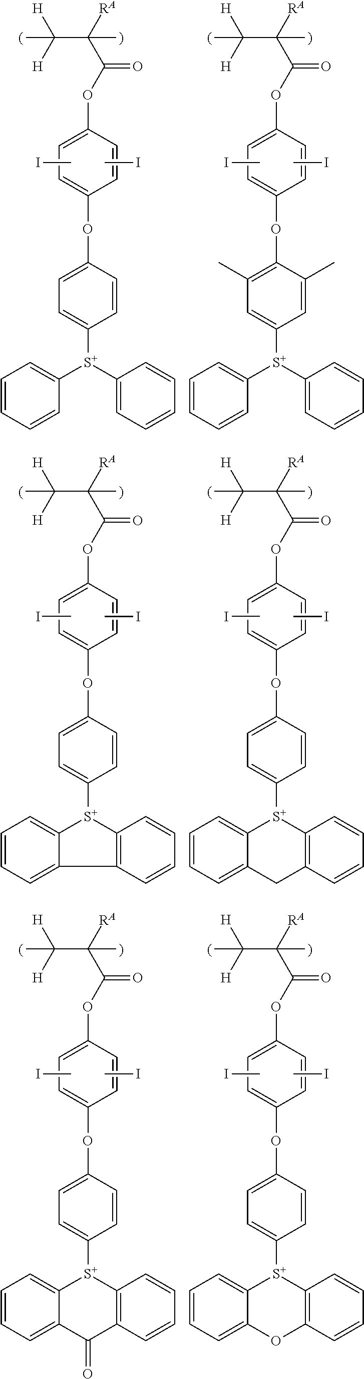

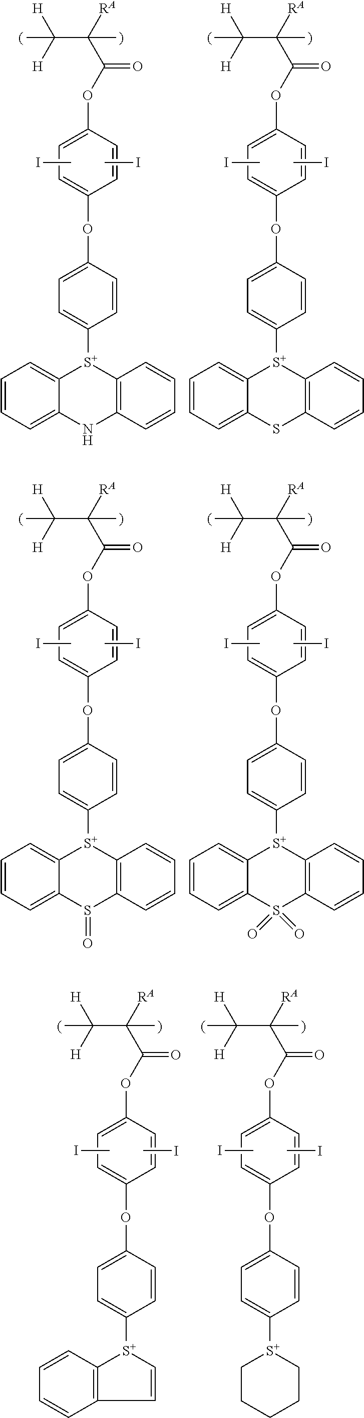

In formula (c1), M⁻ is a non-nucleophilic counter ion. The non-nucleophilic counter ion is preferably a halide ion, a sulfonate anion, an imidate anion, or a methide anion. Specific examples of the halide ion include a chloride ion and a bromide ion. Specific examples of the sulfonate anion (sulfonate ion) include fluoroalkyl sulfonate ions such as triflate ion, 1,1,1-trifluoroethanesulfonate ion, and nonafluorobutanesulfonate ion; aryl sulfonate ions such as tosylate ion, benzenesulfonate ion, 4-fluorobenzenesulfonate ion, and 1,2,3,4,5-pentafluorobenzenesulfonate ion; and alkyl sulfonate ions such as mesylate ion and butanesulfonate ion. Specific examples of the imidate anion (imide ion) include bis(trifluoromethylsulfonyl)imide ion, bis(perfluoroethylsulfonyl)imide ion, and bis(perfluorobutylsulfonyl)imide ion. Specific examples of the methide anion (methide ion) include tris(trifluoromethylsulfonyl)methide ion, and tris(perfluoroethylsulfonyl)methide ion.

Another example of the non-nucleophilic counter ion includes an anion represented by any one of formulae (c1-1) to (c1-4).

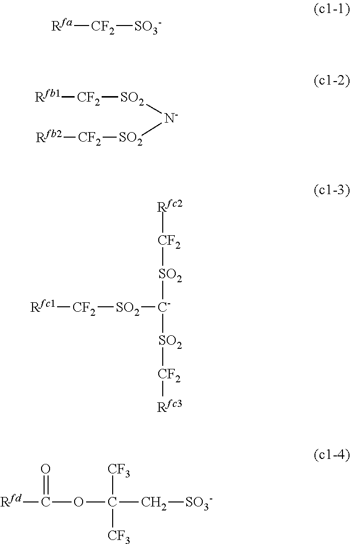

In formula (c1-1), R^{fa} is a fluorine atom or a hydrocarbyl group having 1 to 40 carbon atoms and optionally comprising a heteroatom. The hydrocarbyl group may be either saturated or unsaturated and may be linear, branched, or cyclic. Specific examples thereof include the same hydrocarbyl group represented by R^{fa1} in formula (c1-1-1), which will be described below, as illustrated.

The anion represented by formula (ci-1) is preferably represented by formula (ci-1-1).

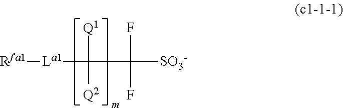

In formula (ci-1-1), Q¹ and Q² are each independently a hydrogen atom, a fluorine atom, or a fluorinated saturated hydrocarbyl group having 1 to 6 carbon atoms. To enhance solvent solubility, at least one of the Q¹ and Q² is preferably a trifluoromethyl group. “m” is 0, 1, 2, 3, or 4, particularly preferably 1. R^{fa1} is a hydrocarbyl group having 1 to 35 carbon atoms and optionally comprising a heteroatom. The heteroatom is preferably, for instance, an oxygen atom, a nitrogen atom, a sulfur atom, and a halogen atom, and more preferably an oxygen atom. The hydrocarbyl group particularly preferably has 6 to 30 carbon atoms from the viewpoint of achieving a high resolution in fine patterning.

In formula (ci-1-1), the hydrocarbyl group having 1 to 35 carbon atoms, represented by R^{fa1}, may be either saturated or unsaturated and linear, branched, or cyclic. Specific examples thereof include alkyl groups having 1 to 35 carbon atoms, such as a methyl group, an ethyl group, an n-propyl group, an isopropyl group, an n-butyl group, an isobutyl group, a sec-butyl group, a tert-butyl group, a pentyl group, a neopentyl group, a hexyl group, a heptyl group, a 2-ethylhexyl group, a nonyl group, an undecyl group, a tridecyl group, a pentadecyl group, a heptadecyl group, and an icosyl group; cyclic saturated hydrocarbyl groups having 3 to 35 carbon atoms, such as a cyclopentyl group, a cyclohexyl group, a 1-adamantyl group, a 2-adamantyl group, a 1-adamantylmethyl group, a norbornyl group, a norbornylmethyl group, a tricyclodecyl group, a tetracyclododecyl group, a tetracyclododecylmethyl group, and a dicyclohexylmethyl group; unsaturated aliphatic hydrocarbyl groups having 2 to 35 carbon atoms, such as a 2-propenyl group and a 3-cyclohexenyl group; aryl groups having 6 to 35 carbon atoms, such as a phenyl group, a 1-naphthyl group, a 2-naphthyl group, and a 9-fluorenyl group; aralkyl groups having 7 to 35 carbon atoms, such as a benzyl group and a diphenylmethyl group; and groups obtained by combining these groups.

Some or all of the hydrogen atoms in the hydrocarbyl group are optionally substituted with a group containing a heteroatom such as an oxygen atom, a sulfur atom, a nitrogen atom, or a halogen atom, and some of —CH₂— in the hydrocarbyl group are optionally substituted with a group containing a heteroatom such as an oxygen atom, a sulfur atom, or a nitrogen atom. As a result, the hydrocarbyl group may contain, for instance, a hydroxy group, a fluorine atom, a chlorine atom, a bromine atom, an iodine atom, a cyano group, a nitro group, a carbonyl group, an ether bond, an ester bond, a sulfonate ester bond, a carbonate bond, a lactone ring, a sultone ring, a carboxylic anhydride (—C(═O)—O—C(═O)—), and a haloalkyl group. Specific examples of the hydrocarbyl group containing a heteroatom include a tetrahydrofuryl group, a methoxymethyl group, an ethoxymethyl group, a methylthiomethyl group, an acetamidomethyl group, a trifluoroethyl group, a (2-methoxyethoxy)methyl group, an acetoxymethyl group, a 2-carboxy-1-cyclohexyl group, a 2-oxopropyl group, a 4-oxo-1-adamantyl group, and a 3-oxocyclohexyl group.

In formula (c1-1-1), L^{A1} is a single bond, an ether bond, an ester bond, a sulfonate ester bond, a carbonate bond, or a carbamate bond. From the viewpoint of synthesis, the L^{A1} is preferably an ether bond or an ester bond, and further preferably an ester bond.

Specific examples of the anion represented by formula (c1-1) are given below, but not limited thereto. In formulae below, Q¹ is the same as above, and Ac represents an acetyl group.

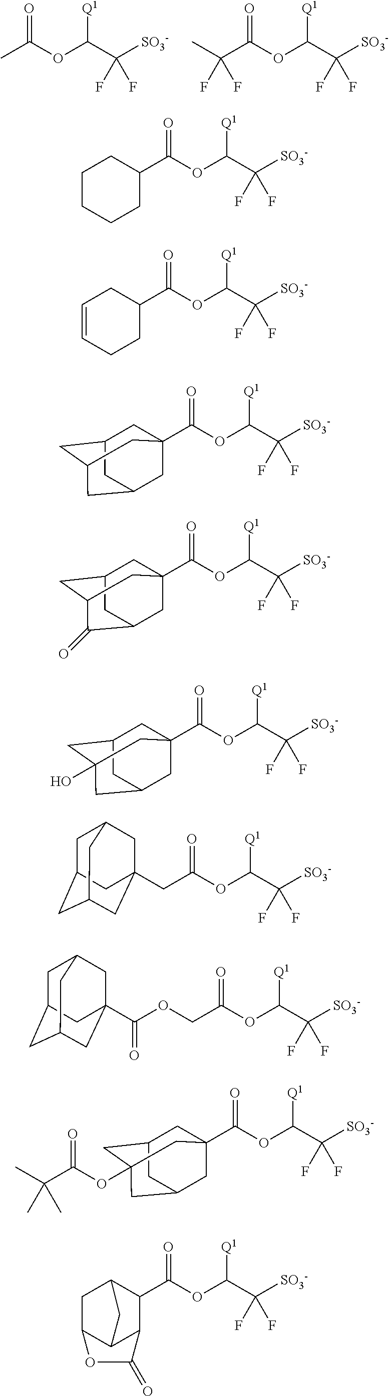

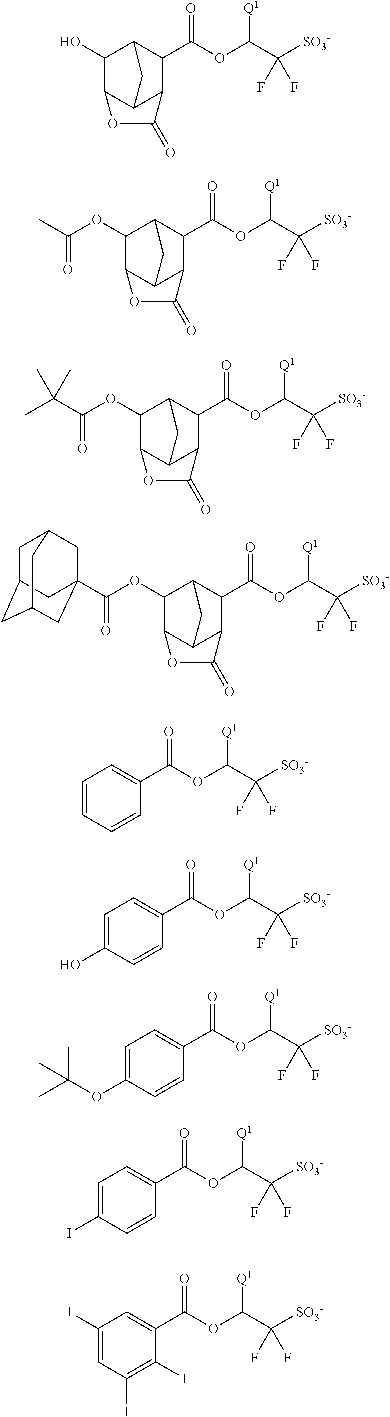

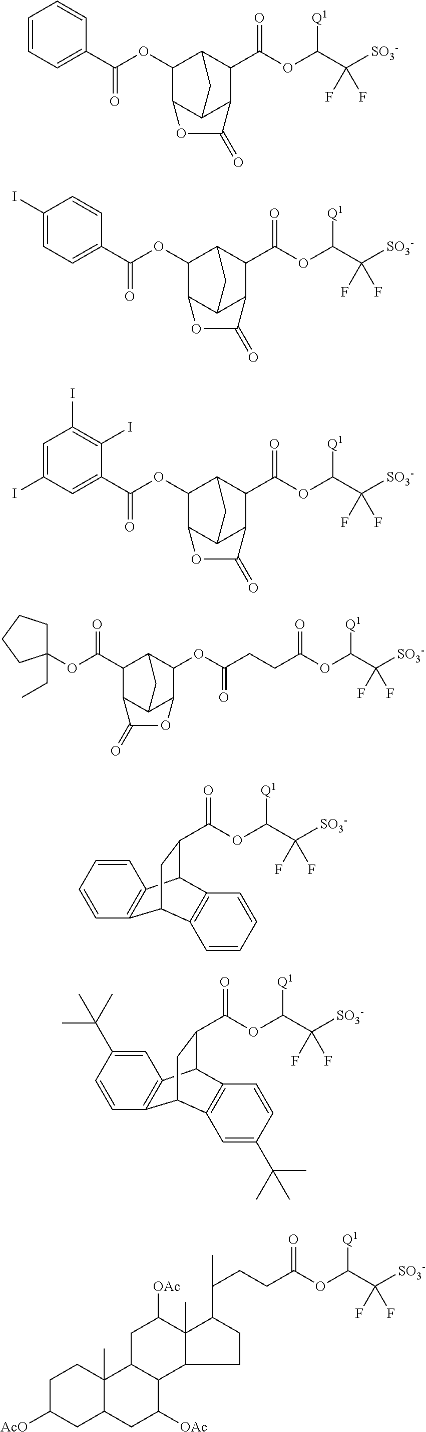

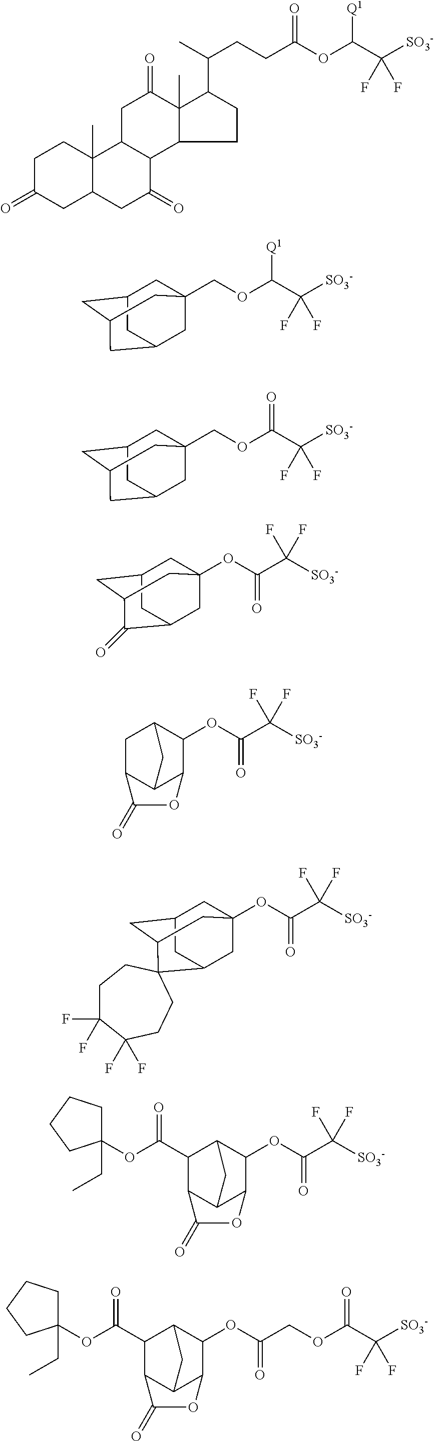

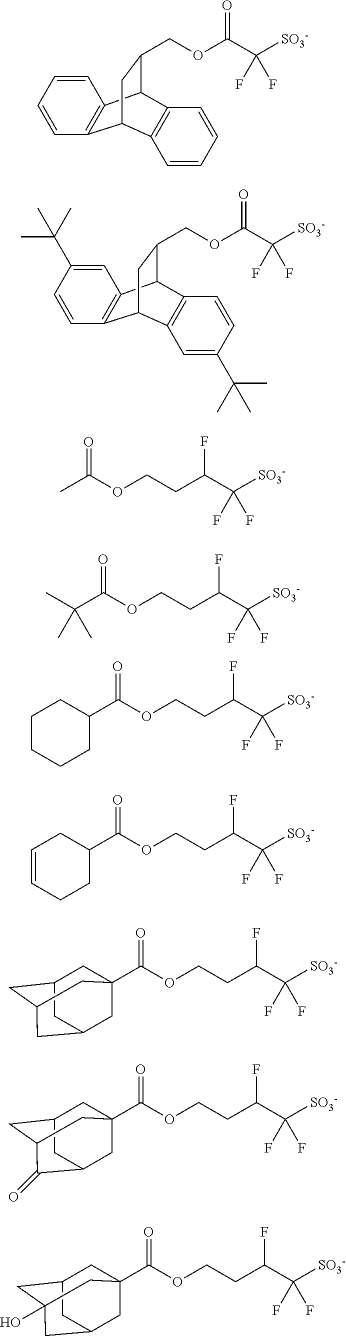

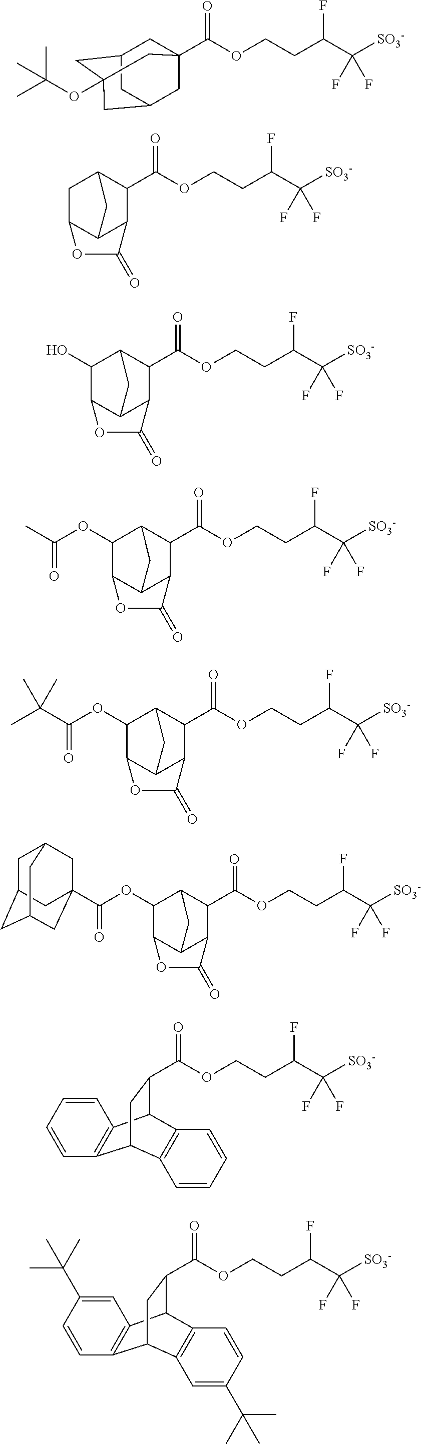

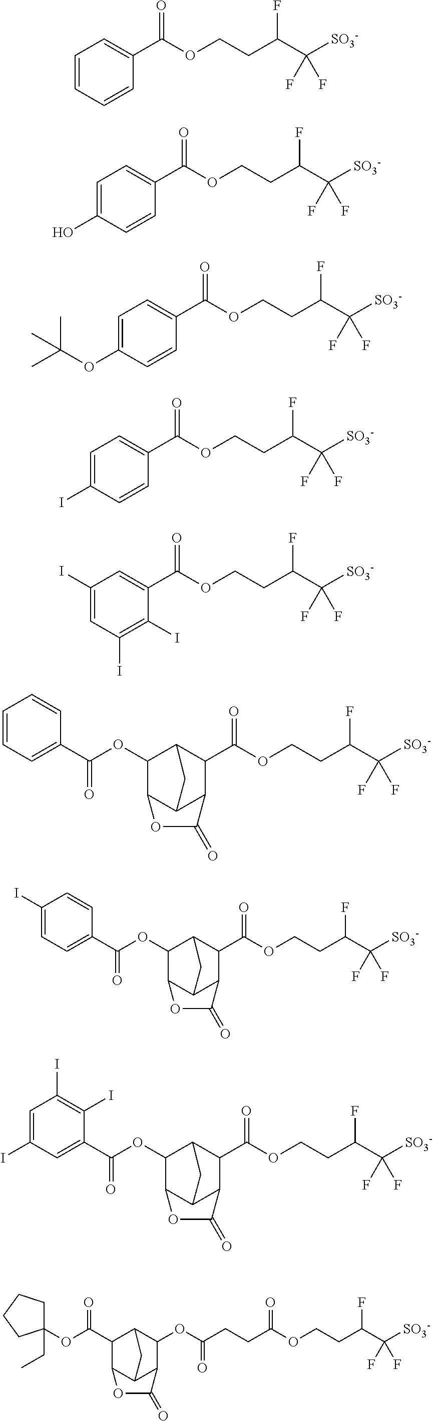

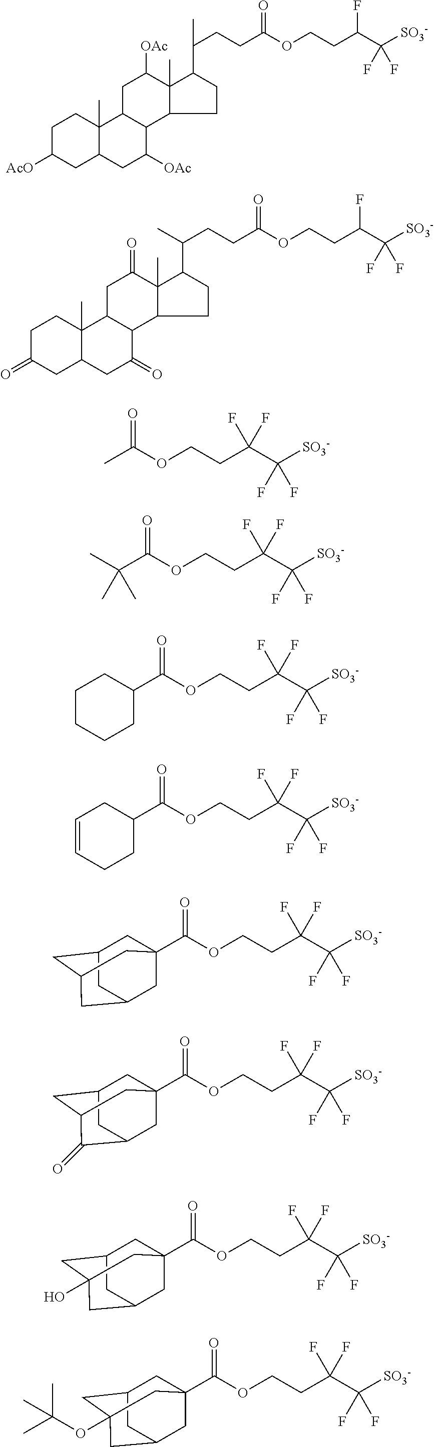

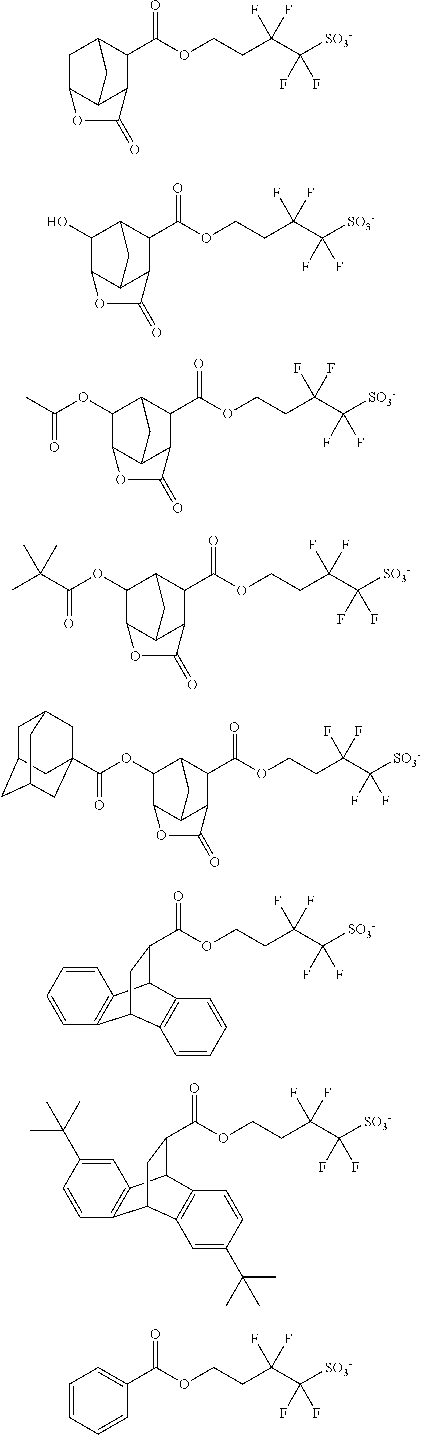

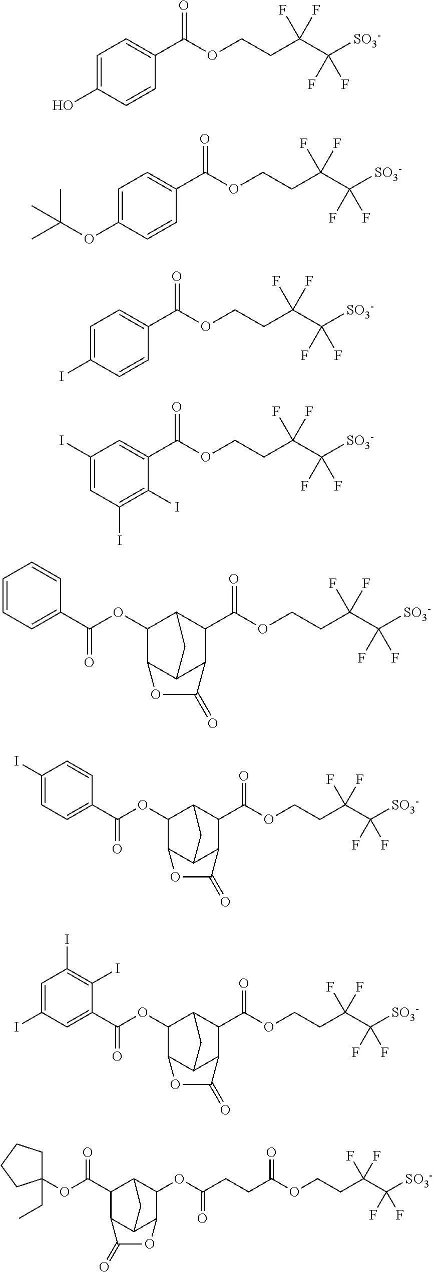

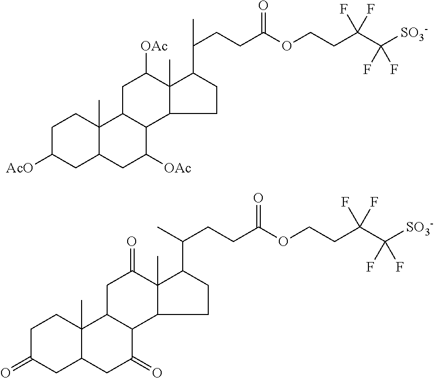

In formula (c1-2), R^{fb1} and R^{fb2} are each independently a fluorine atom or a hydrocarbyl group having 1 to 40 carbon atoms and optionally comprising a heteroatom. The hydrocarbyl group may be either saturated or unsaturated and may be linear, branched, or cyclic. Specific examples thereof include the same hydrocarbyl group represented by R^{fa1} in formula (c1-1-1) as illustrated. R^{fb1} and R^{fb2} are each preferably a fluorine atom or a linear fluorinated alkyl group having 1 to 4 carbon atoms. In addition, R^{fb1} and R^{fb2} are optionally bonded to each other to form a ring together with a group (—CF₂—SO₂—N⁻—SO₂—CF₂—) to which the R^{fb1} and R^{fb2} are bonded. In this case, the group obtained by bonding R^{fb1} and R^{fb2} to each other is preferably a fluorinated ethylene group or a fluorinated propylene group.

In formula (c1-3), R^{fc1}, R^{fc2}, and R^{fc3} are each independently a fluorine atom or a hydrocarbyl group having 1 to 40 carbon atoms and optionally comprising a heteroatom. The hydrocarbyl group may be either saturated or unsaturated and may be linear, branched, or cyclic. Specific examples thereof include the same hydrocarbyl group represented by R^{fa1} in formula (c1-1-1) as illustrated. R^{fc1}, R^{fc2}, and R^{fc3} are each preferably a fluorine atom or a linear fluorinated alkyl group having 1 to 4 carbon atoms. In addition, R^{fc1} and R^{fc2} are optionally bonded to each other to form a ring together with a group (—CF₂—SO₂—C⁻—SO₂—CF₂—) to which the R^{fc1} and R^{fc2} are bonded. In this case, the group obtained by bonding R^{fc1} and R^{fc2} to each other is preferably a fluorinated ethylene group or a fluorinated propylene group.

In formula (c1-4), R^{fd} is a hydrocarbyl group having 1 to 40 carbon atoms and optionally comprising a heteroatom. The hydrocarbyl group may be either saturated or unsaturated and may be linear, branched, or cyclic. Specific examples thereof include the same hydrocarbyl group represented by R^{fa1} in formula (c1-1-1) as illustrated.

Specific examples of the anion represented by formula (c1-4) are given below, but not limited thereto.

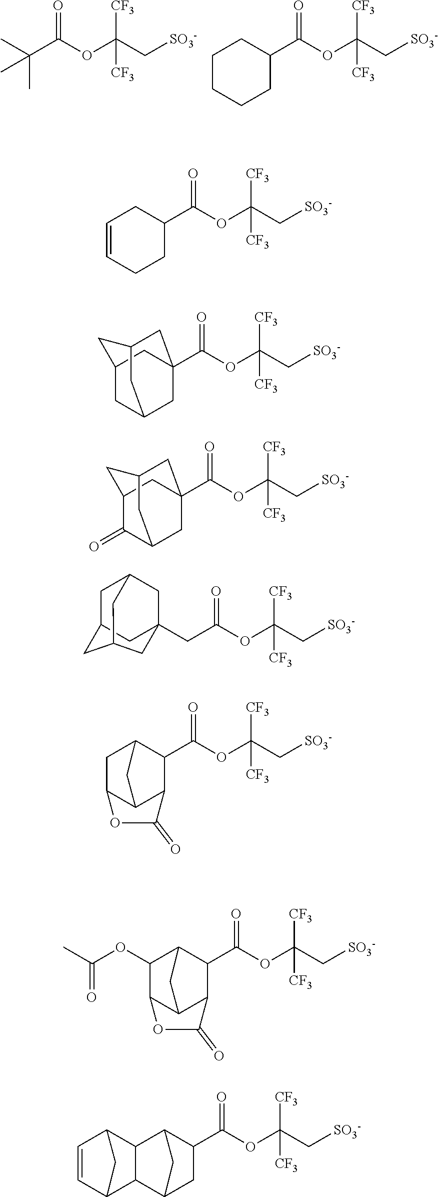

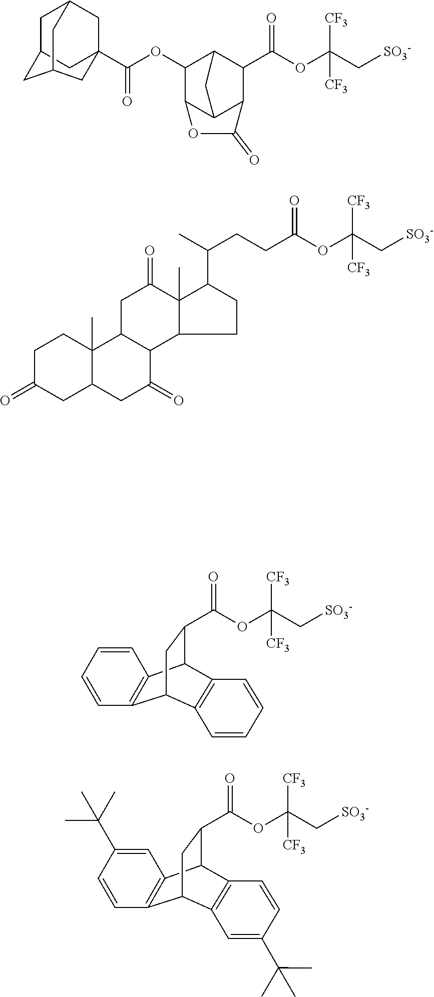

In addition, examples of the non-nucleophilic counter ion include an anion having an aromatic ring substituted with an iodine atom or a bromine atom. Specific examples of such an anion include anions represented by formula (c1-5).

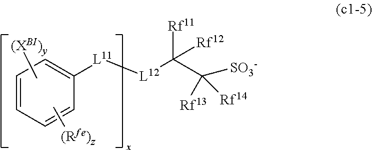

In formula (c1-5), “x” is 1, 2, or 3, “y” is 1, 2, 3, 4, or 5, and “Z” is 0, 1, 2, or 3, provided that 1 y+z≤5. “y” is preferably 1, 2, or 3, and more preferably 2 or 3. “z” is preferably 0, 1, or 2.

In formula (c1-5), X^{BI} is an iodine atom or a bromine atom, wherein when “x” and/or “y” is 2 or more, X^{BI} are the same as or different from each other.

In formula (c1-5), L¹¹ is a single bond, an ether bond, or an ester bond, or a saturated hydrocarbylene group having 1 to 6 carbon atoms and optionally comprising an ether bond or an ester bond. The saturated hydrocarbylene group may be linear, branched, or cyclic.

In formula (c1-5), L¹² is a single bond or a divalent linking group having 1 to 20 carbon atoms when “x” is 1, and a (x+1)-valent linking group having 1 to 20 carbon atoms when “x” is 2 or 3, where the linking group may include an oxygen atom, a sulfur atom, or a nitrogen atom.

In formula (c1-5), R^{fe} is a hydroxy group, a carboxy group, a fluorine atom, a chlorine atom, a bromine atom, or an amino group; a hydrocarbyl group having 1 to 20 carbon atoms, hydrocarbyloxy group having 1 to 20 carbon atoms, hydrocarbylcarbonyl group having 2 to 20 carbon atoms, hydrocarbyloxycarbonyl group having 2 to 20 carbon atoms, hydrocarbylcarbonyloxy group having 2 to 20 carbon atoms, or hydrocarbylsulfonyloxy group having 1 to 20 carbon atoms and optionally comprising a fluorine atom, a chlorine atom, a bromine atom, a hydroxy group, an amino group, or an ether bond; or —N(R^{feA})(R^{feB}), —N(R^{feC})—C(═O)—R^{feD}, or —N(R^{feC})—C(═O)—O—R^{feD}. R^{feA} and R^{feB} are each independently a hydrogen atom or a saturated hydrocarbyl group having 1 to 6 carbon atoms. R^{feC} is a hydrogen atom or a saturated hydrocarbyl group having 1 to 6 carbon atoms and optionally comprises a halogen atom, a hydroxy group, a saturated hydrocarbyloxy group having 1 to 6 carbon atoms, a saturated hydrocarbylcarbonyl group having 2 to 6 carbon atoms, or a saturated hydrocarbylcarbonyloxy group having 2 to 6 carbon atoms. R^{feD} is an aliphatic hydrocarbyl group having 1 to 16 carbon atoms, an aryl group having 6 to 12 carbon atoms, or an aralkyl group having 7 to 15 carbon atoms and optionally comprises a halogen atom, a hydroxy group, a saturated hydrocarbyloxy group having 1 to 6 carbon atoms, a saturated hydrocarbylcarbonyl group having 2 to 6 carbon atoms, or a saturated hydrocarbylcarbonyloxy group having 2 to 6 carbon atoms. The aliphatic hydrocarbyl group may be either saturated or unsaturated and linear branched, or cyclic. The hydrocarbyl group, hydrocarbyloxy group, hydrocarbylcarbonyl group, hydrocarbyloxycarbonyl group, hydrocarbylcarbonyloxy group, and hydrocarbylsulfonyloxy group may be linear, branched, or cyclic. When “x” and/or “z” is 2 or more, R^{fe} are the same as or different from each other.

Of these, R^{fe} is preferably, for instance, a hydroxy group, —N(R^{feC})—C(═O)—R^{feD}, —N(R^{feC})—C(═O)—O—R^{feD}, a fluorine atom, a chlorine atom, a bromine atom, a methyl group, or a methoxy group.

In formula (c1-5), Rf¹¹ to Rf¹⁴ are each independently a hydrogen atom, a fluorine atom, or a trifluoromethyl group, where at least one of the Rf¹¹ to Rf¹⁴ is a fluorine atom or a trifluoromethyl group. Rf¹¹ and Rf¹² may also combine to form a carbonyl group. In particular, both Rf¹³ and Rf¹⁴ are preferably fluorine atoms.

Specific examples of the anion represented by formula (c1-5) are given below, but not limited thereto. In formulae below, X^{B1} is the same as above.

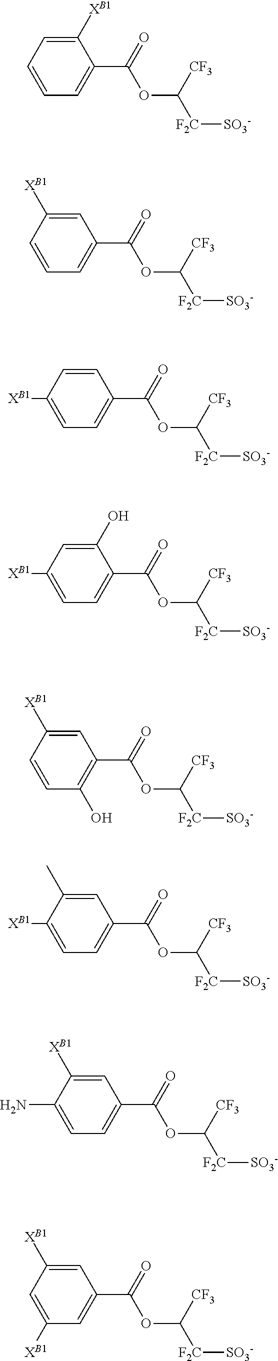

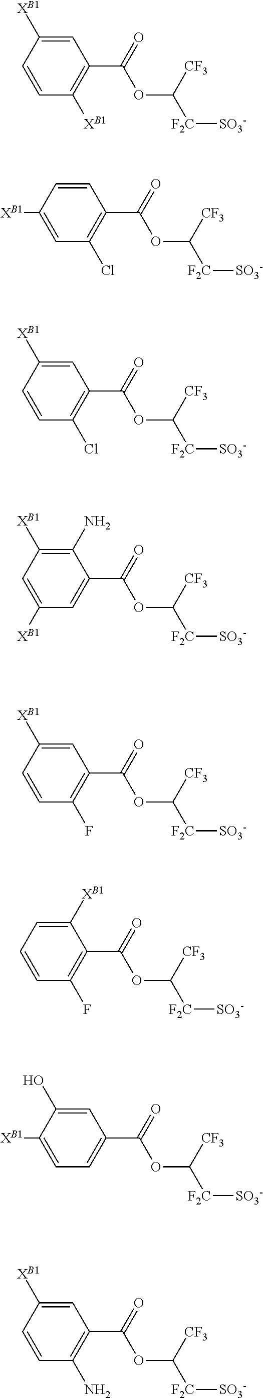

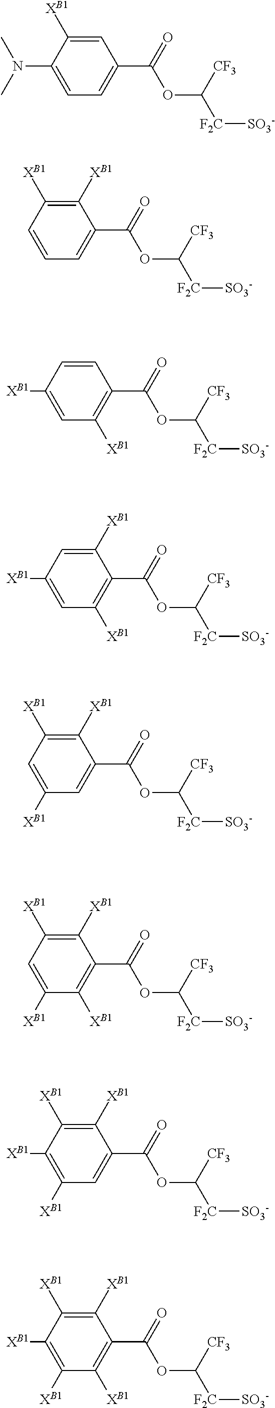

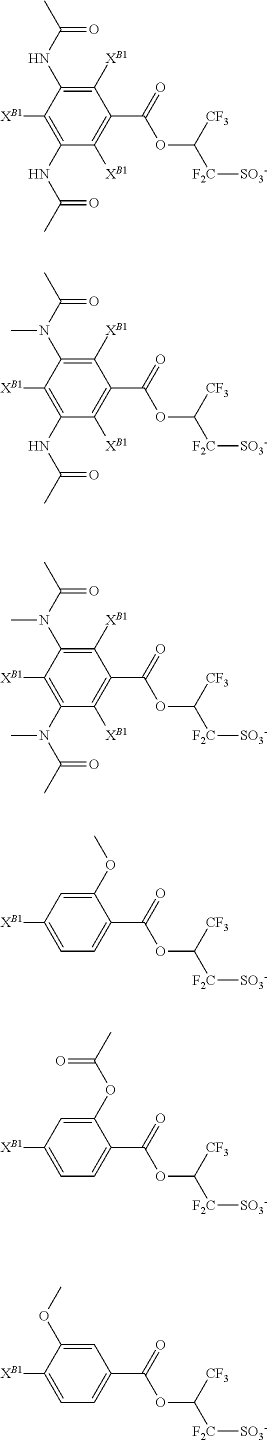

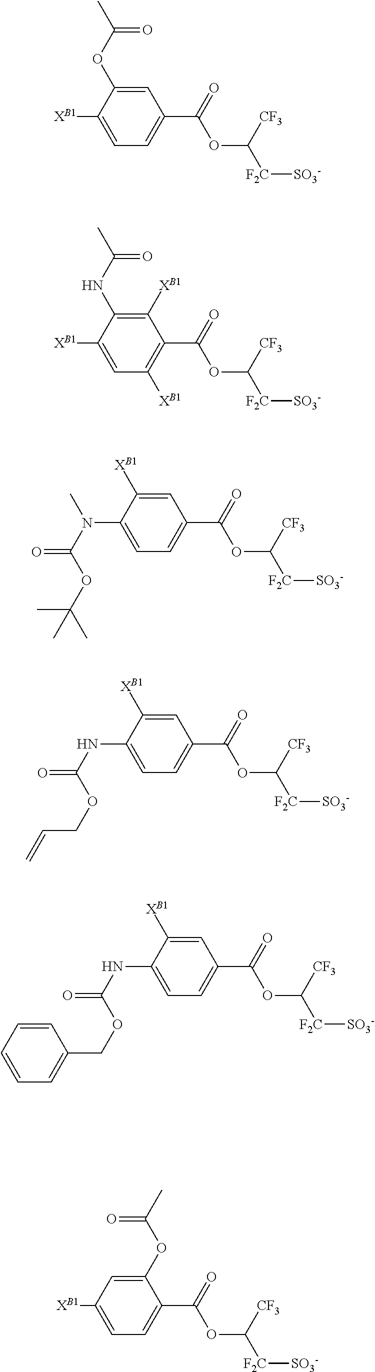

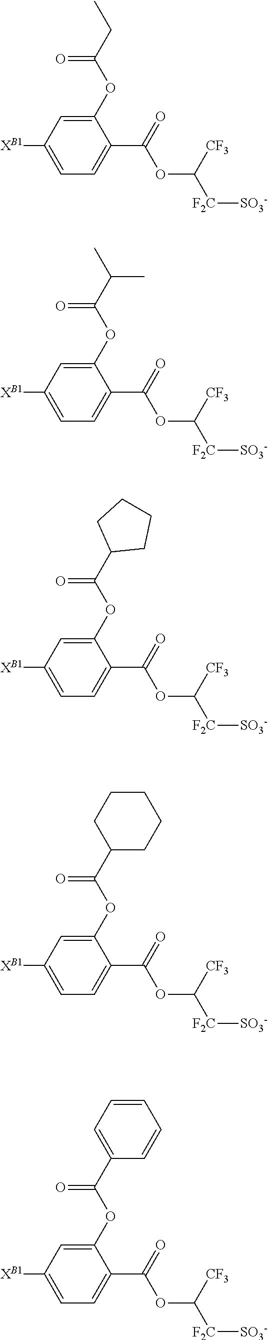

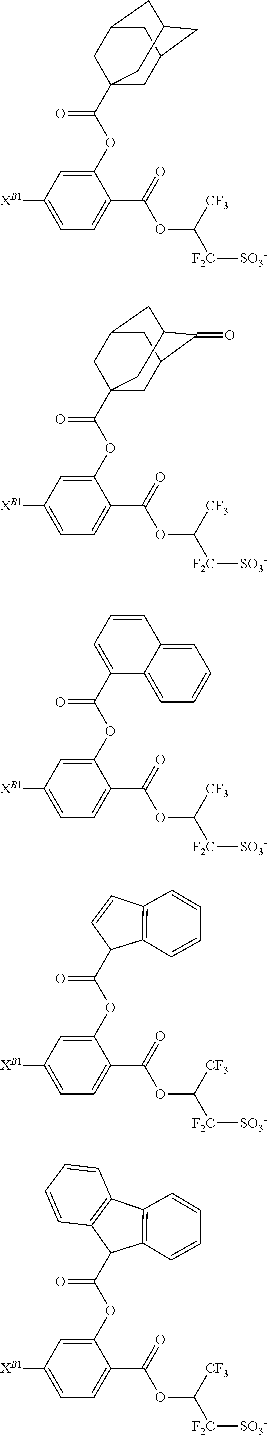

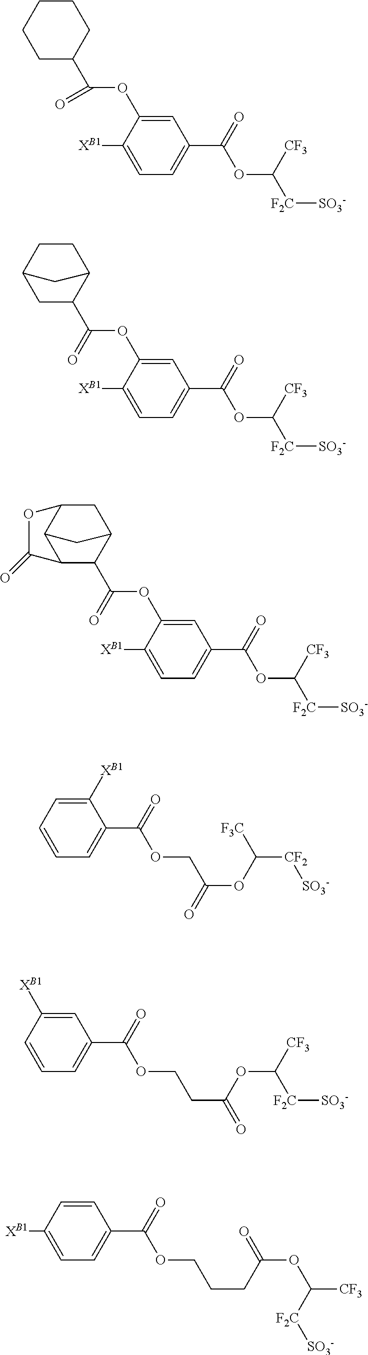

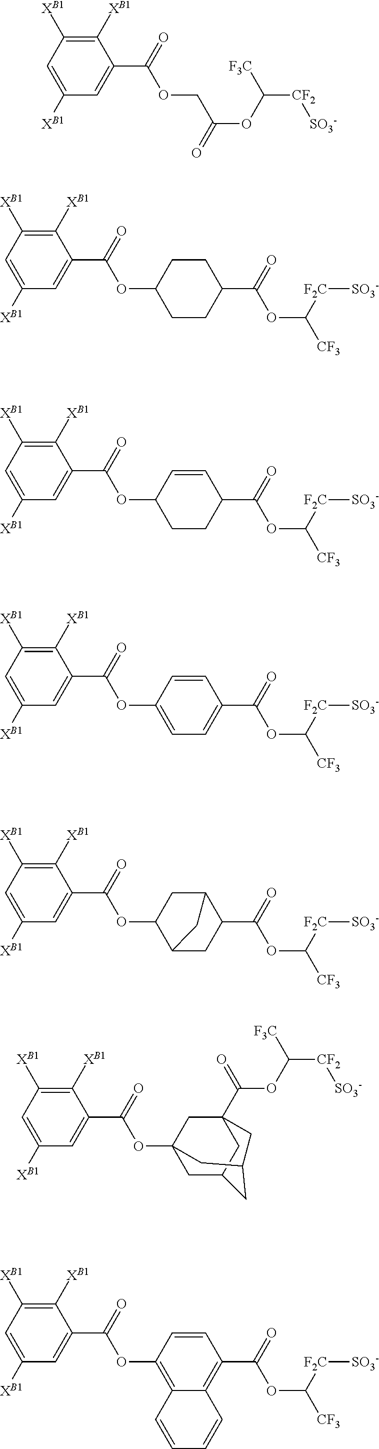

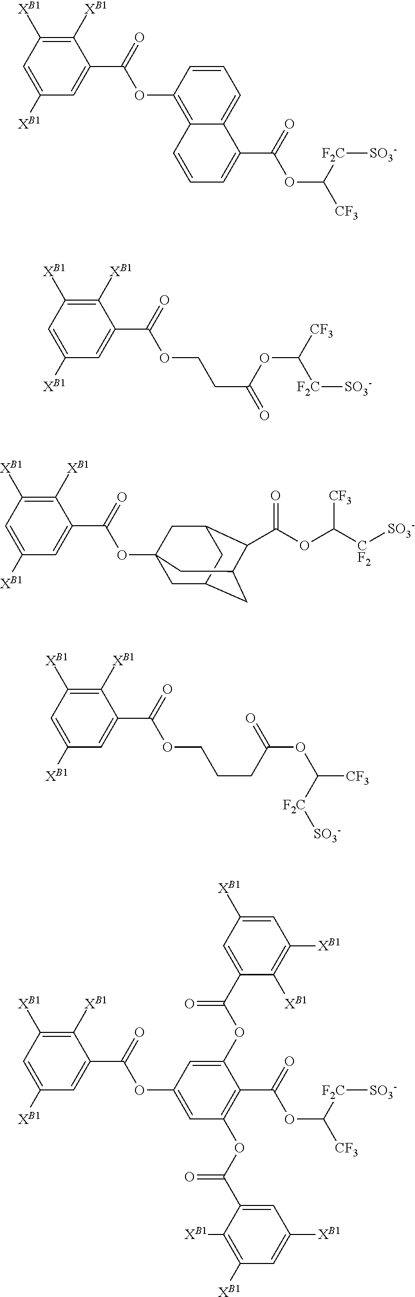

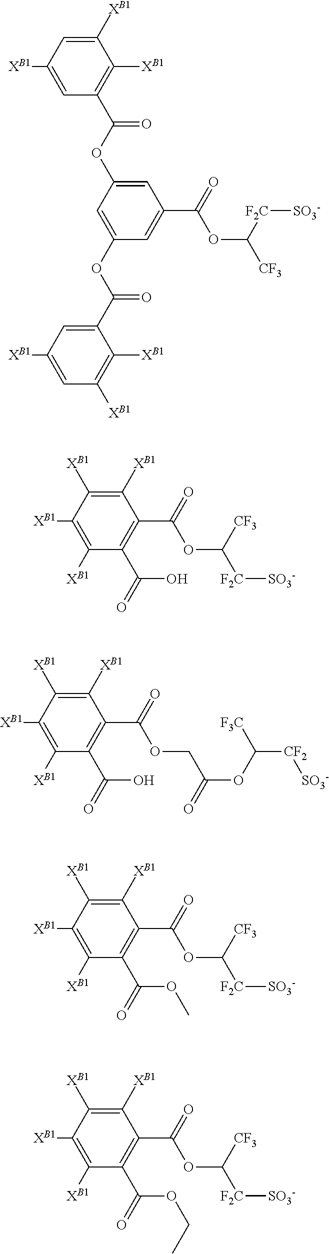

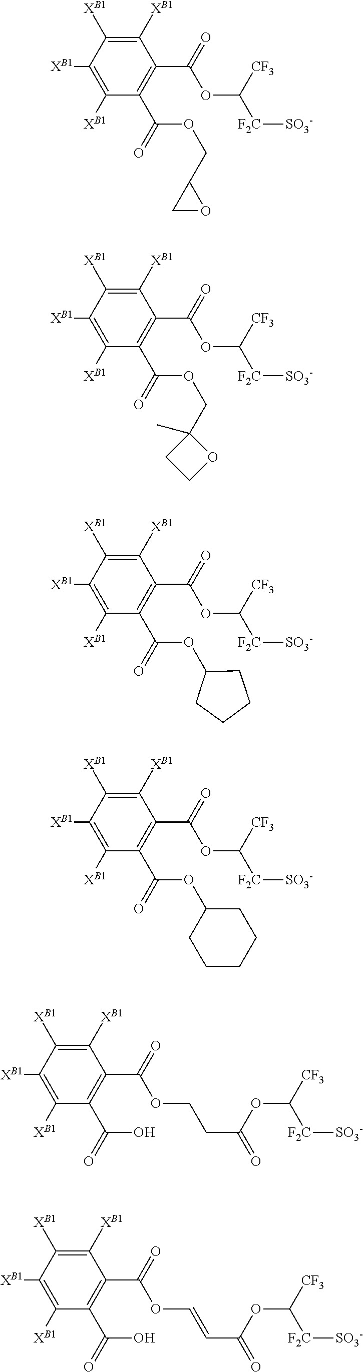

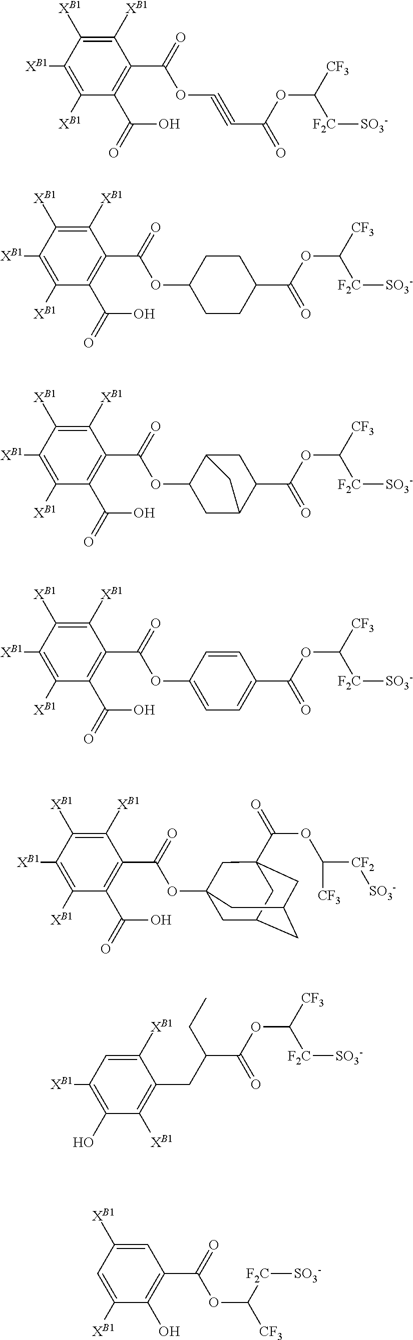

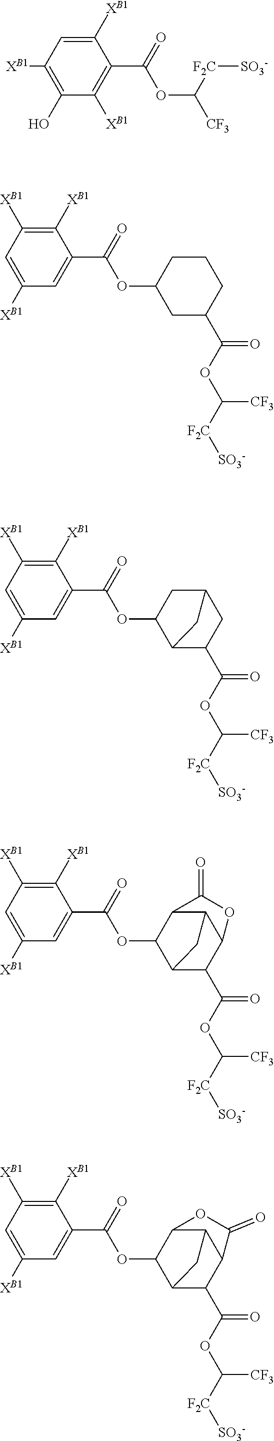

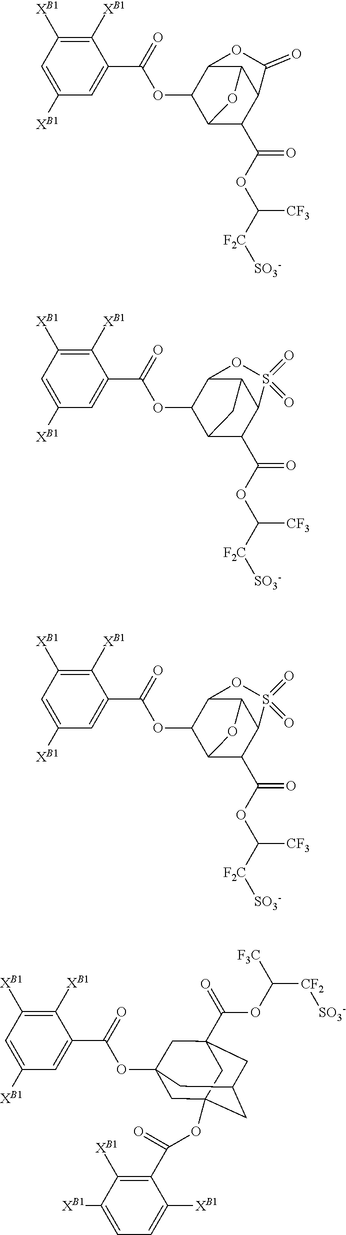

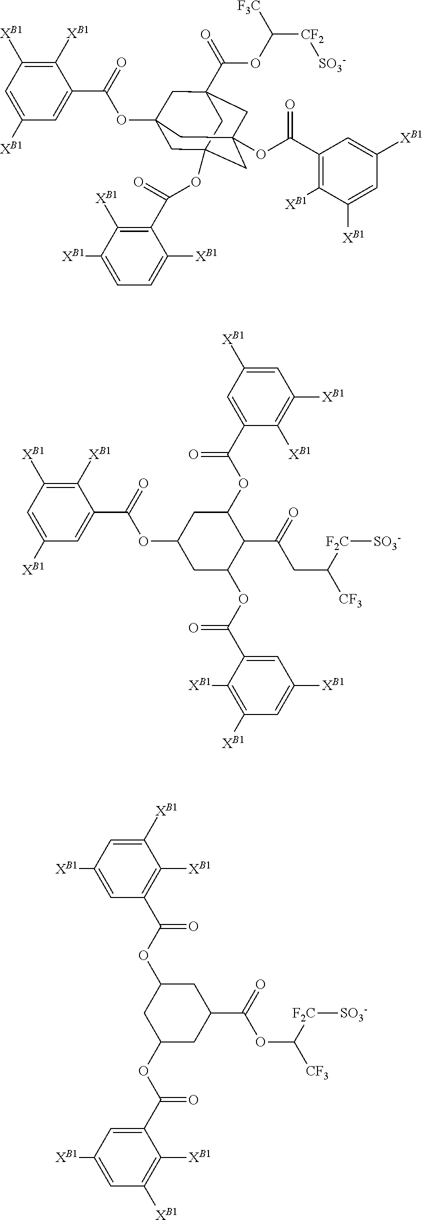

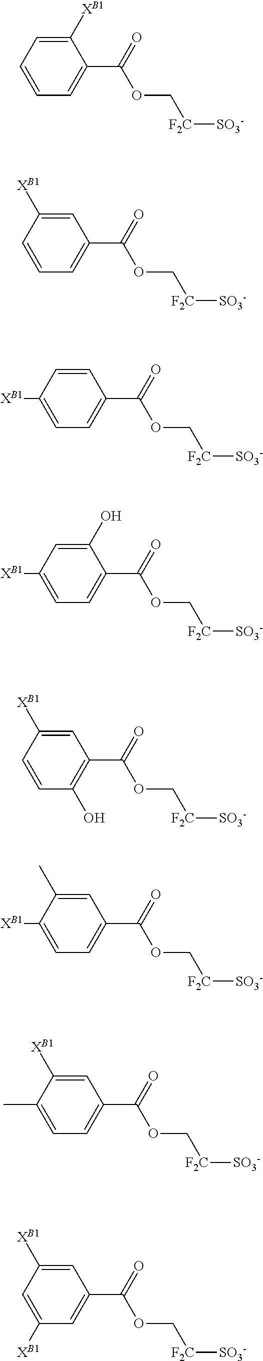

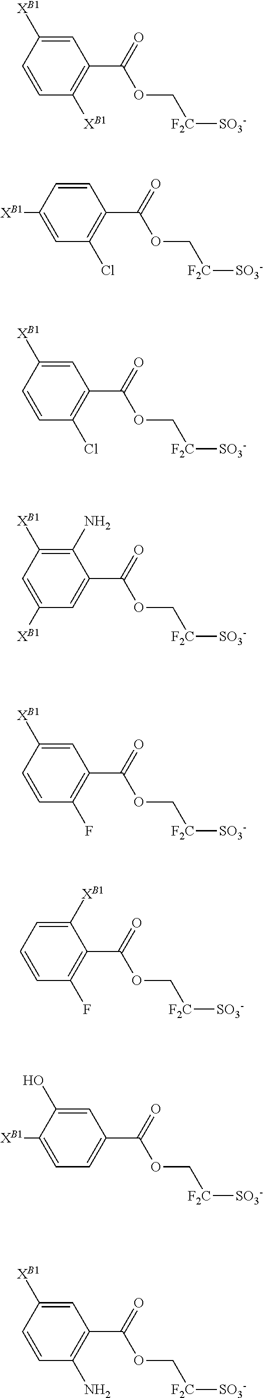

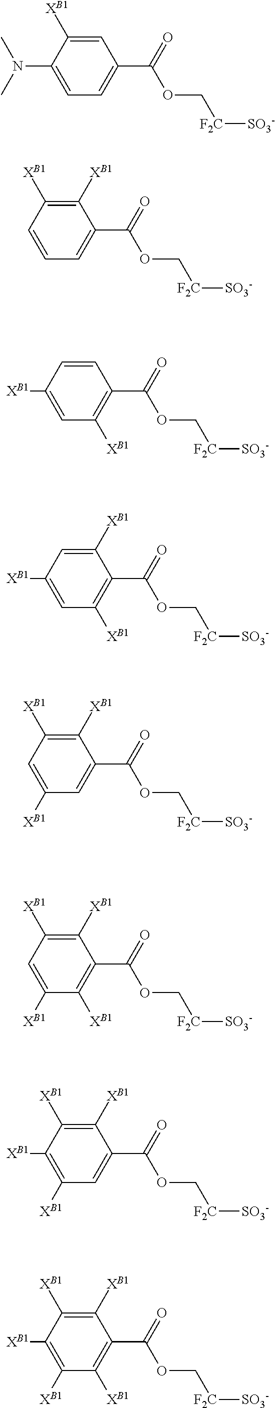

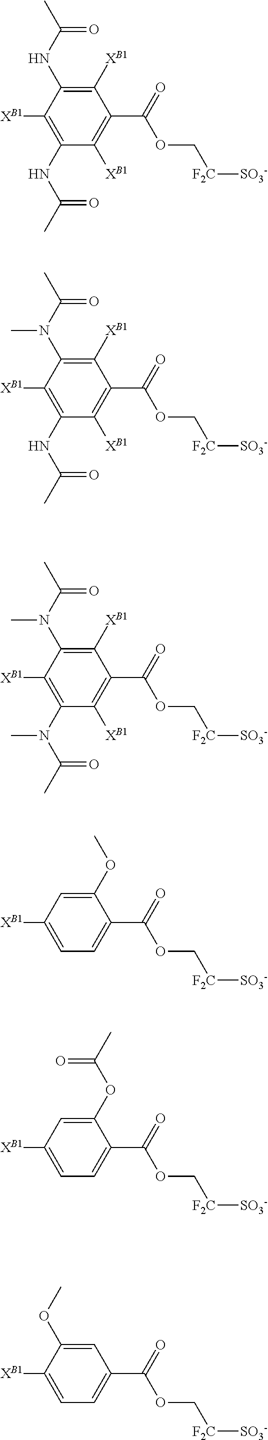

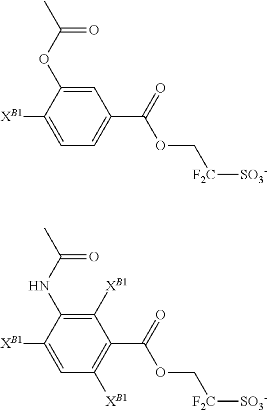

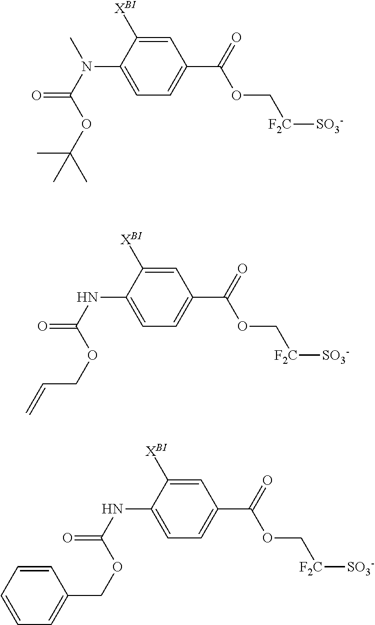

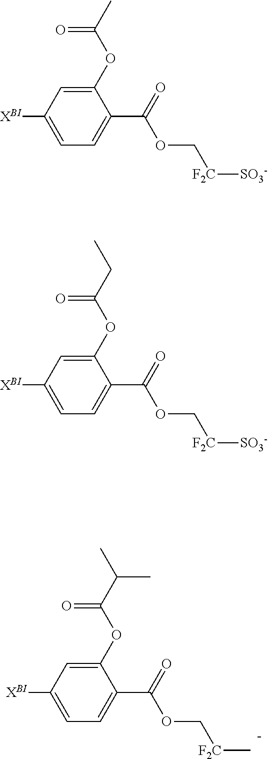

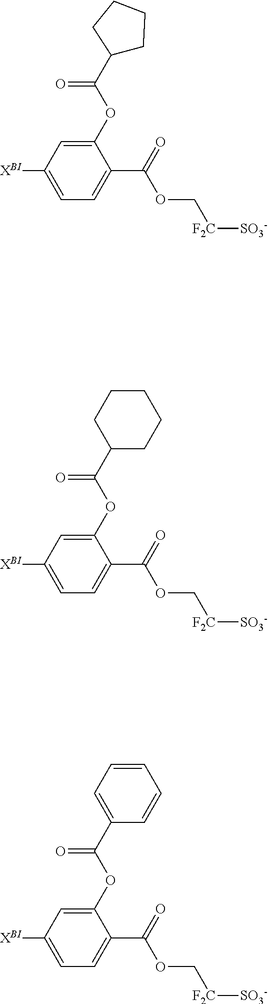

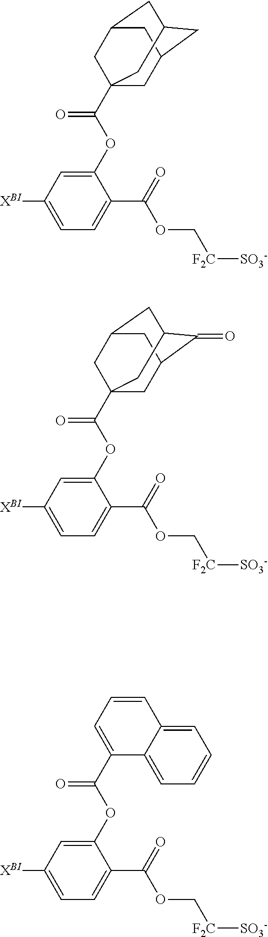

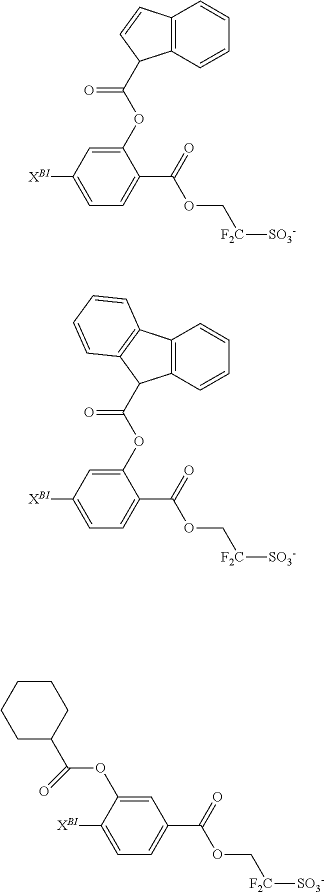

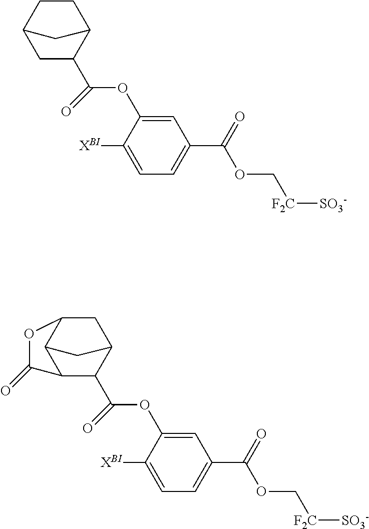

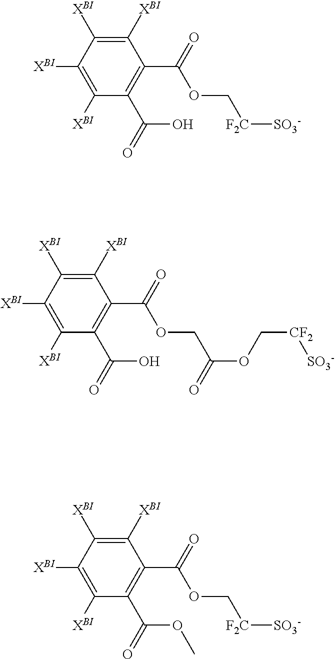

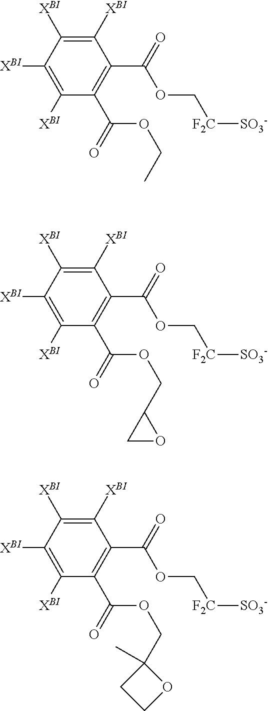

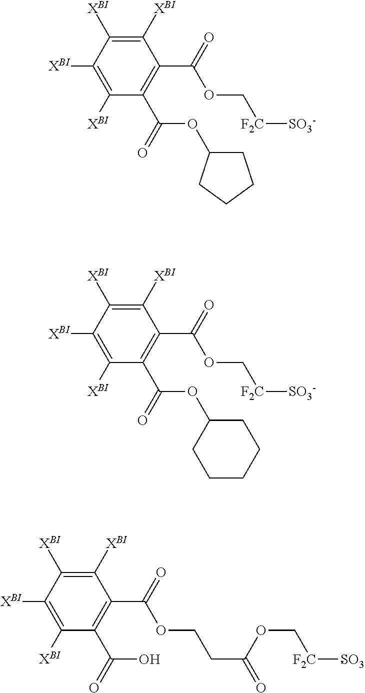

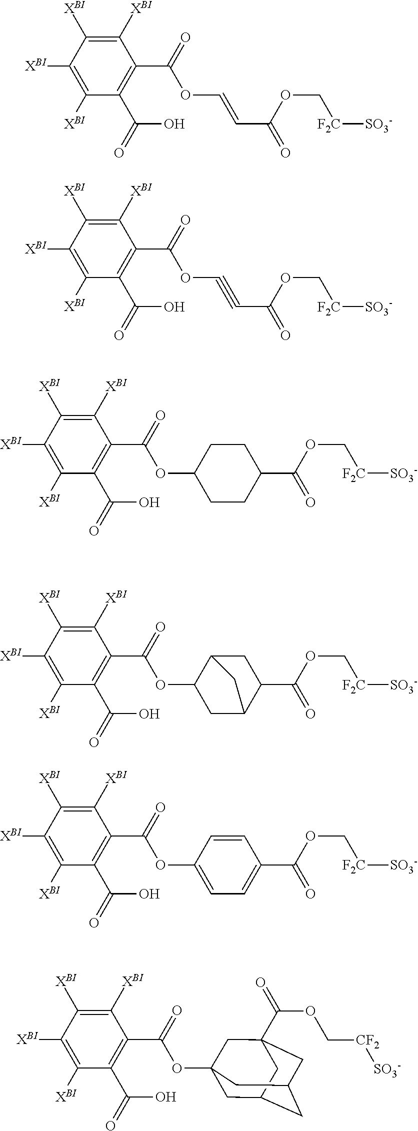

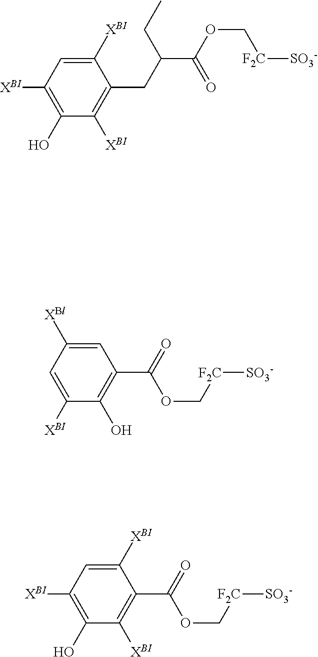

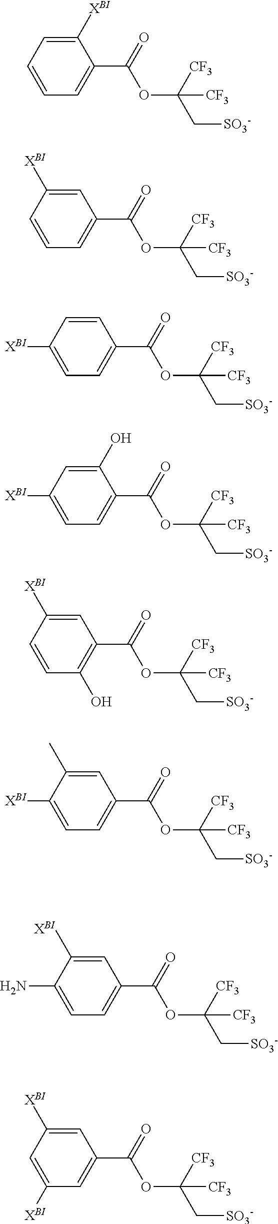

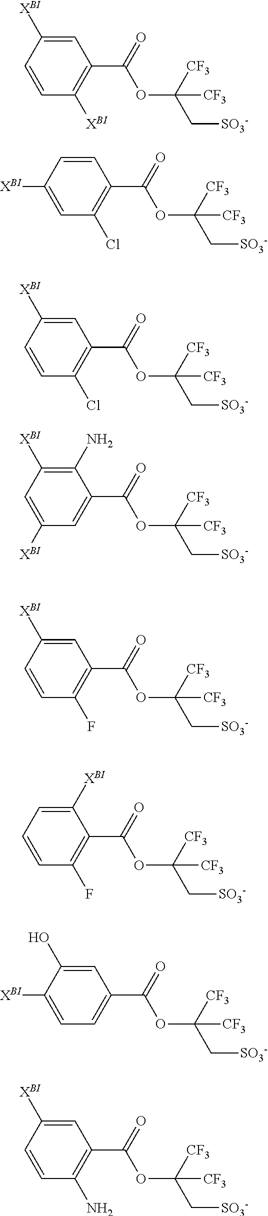

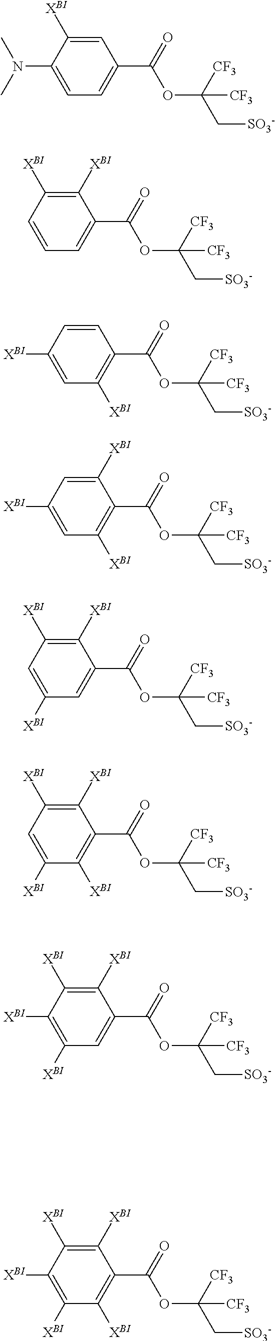

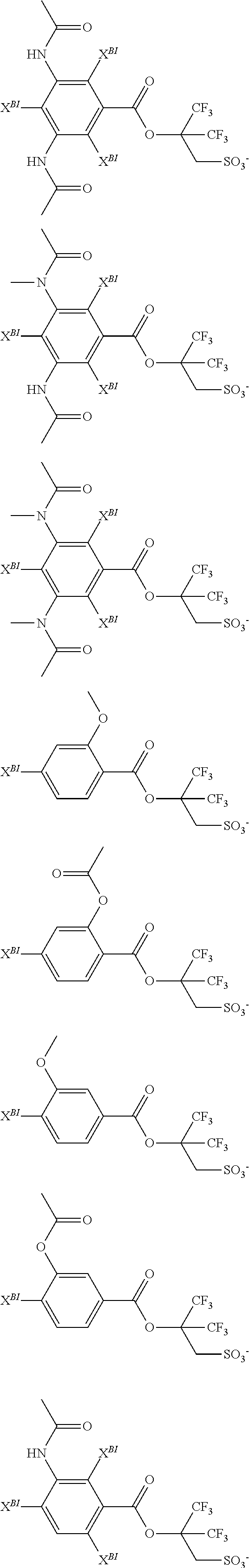

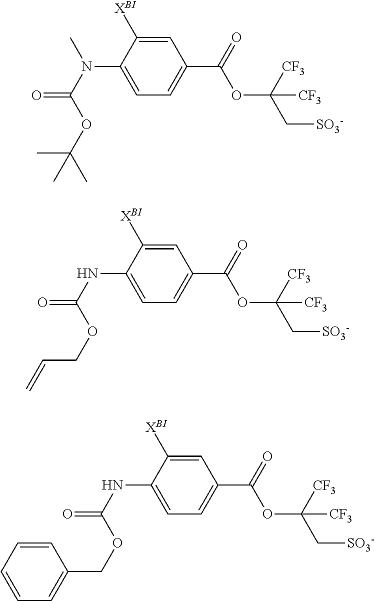

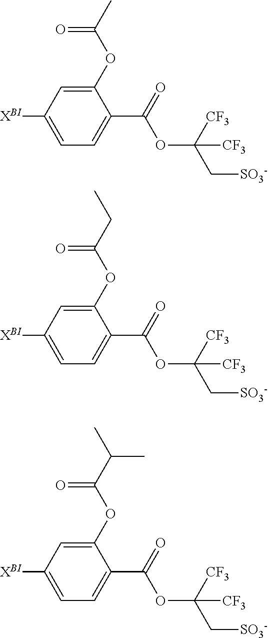

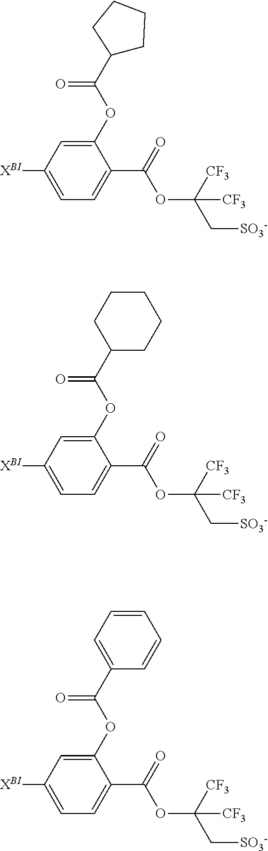

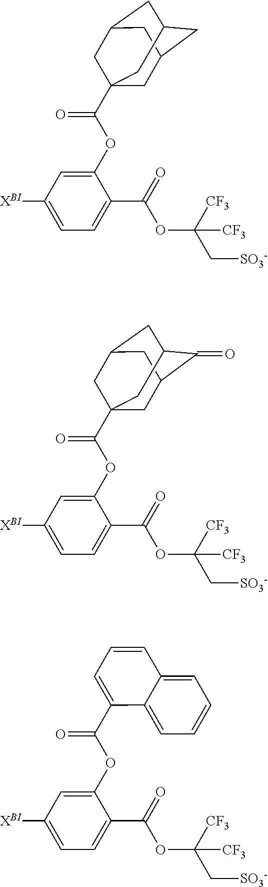

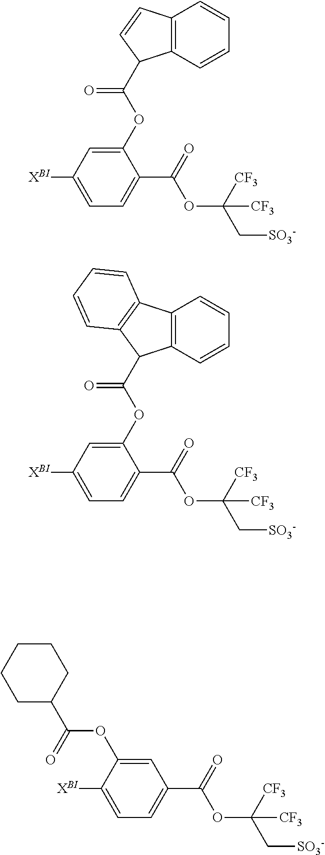

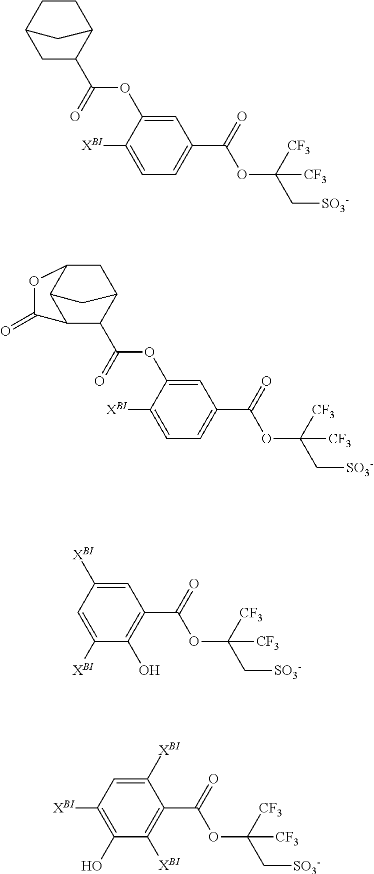

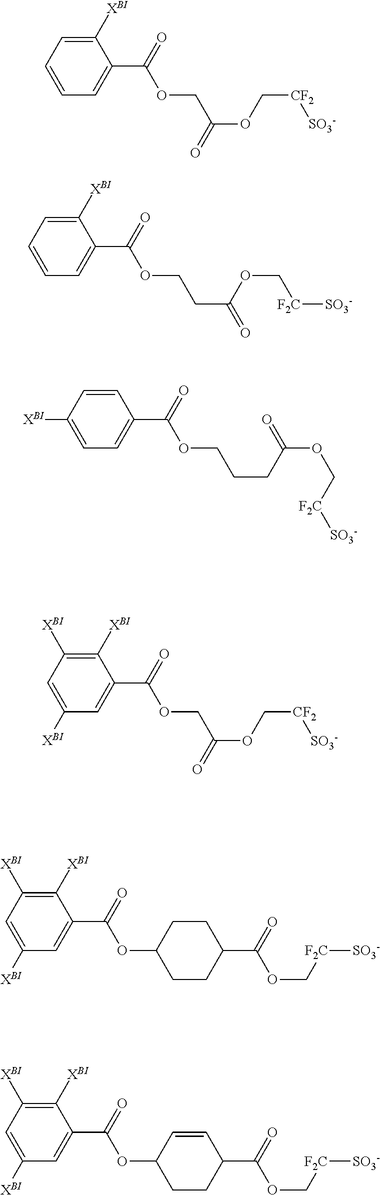

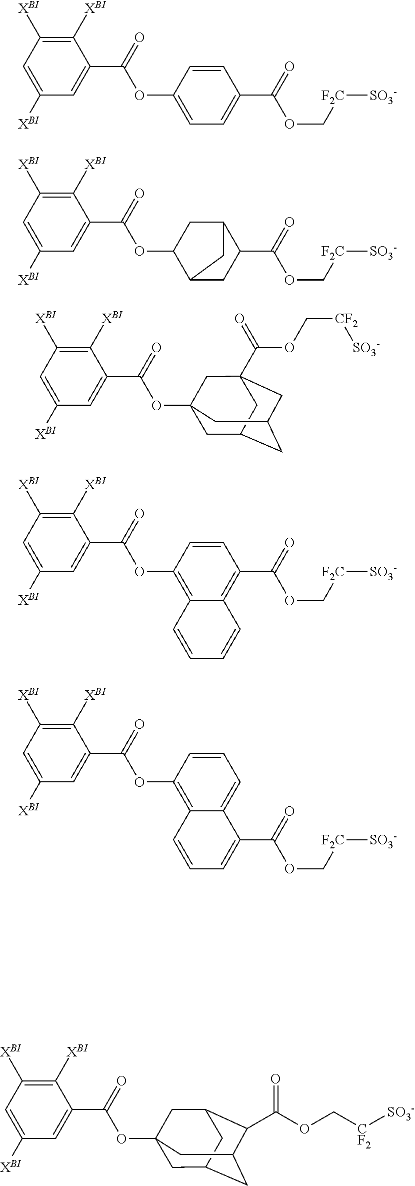

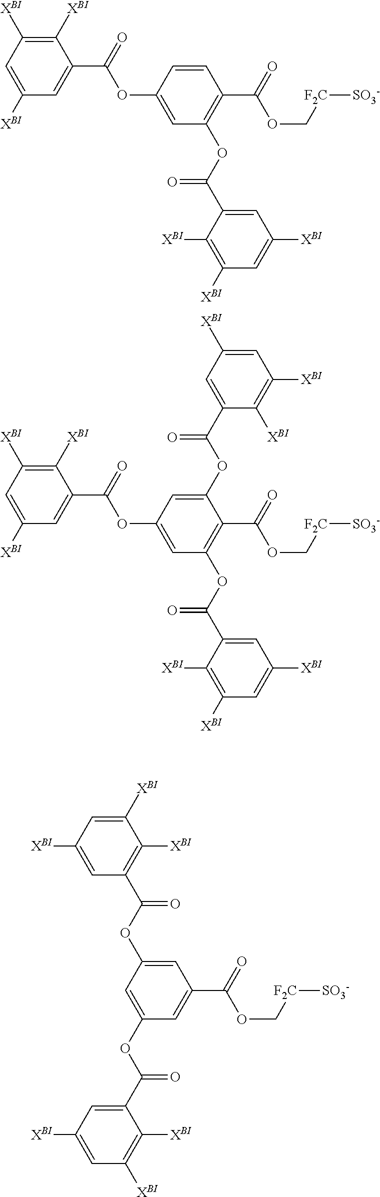

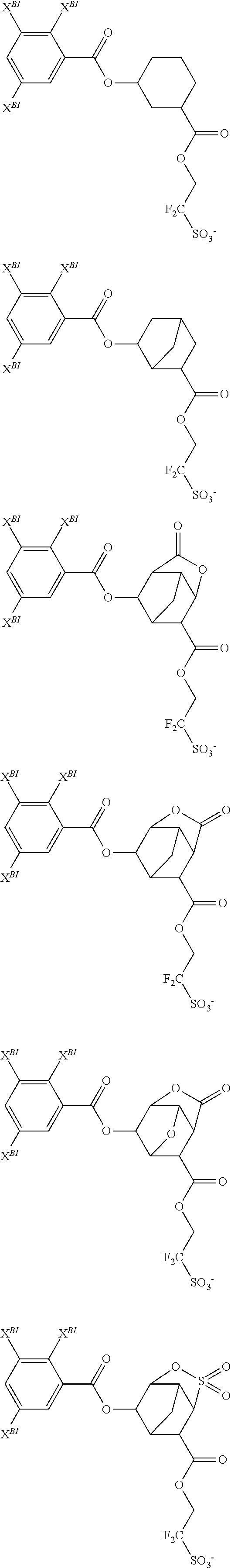

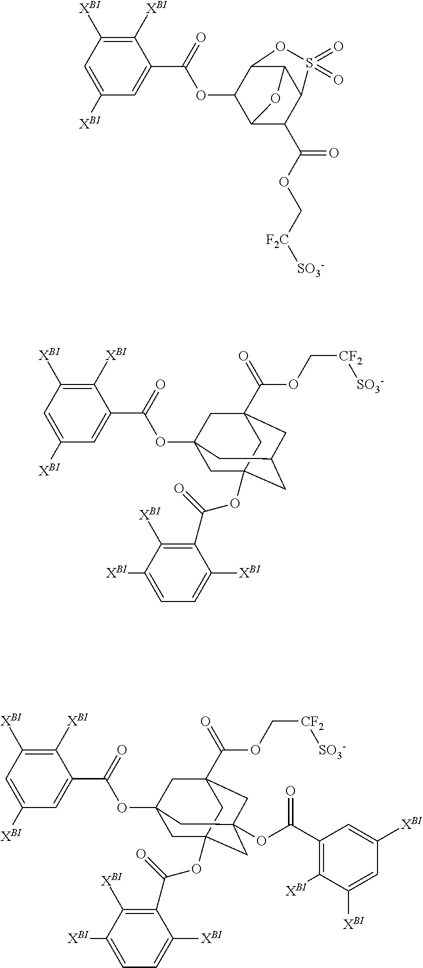

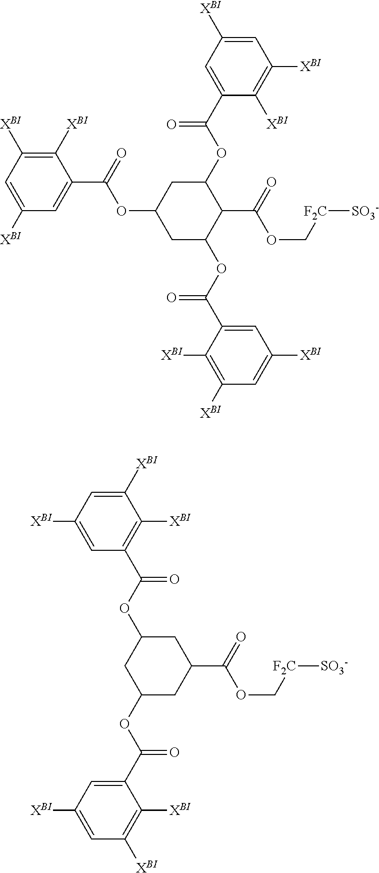

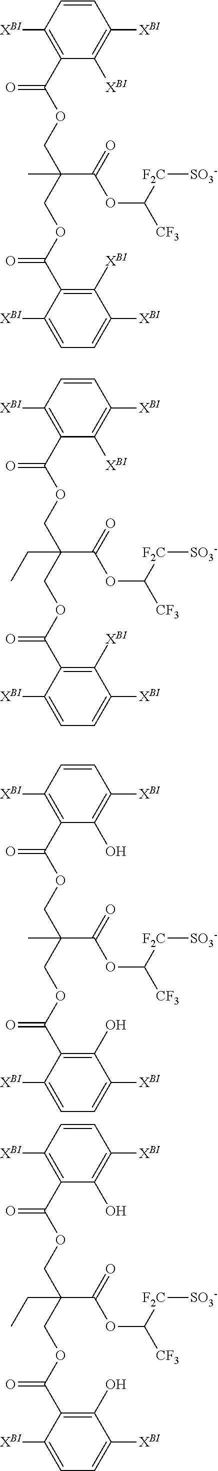

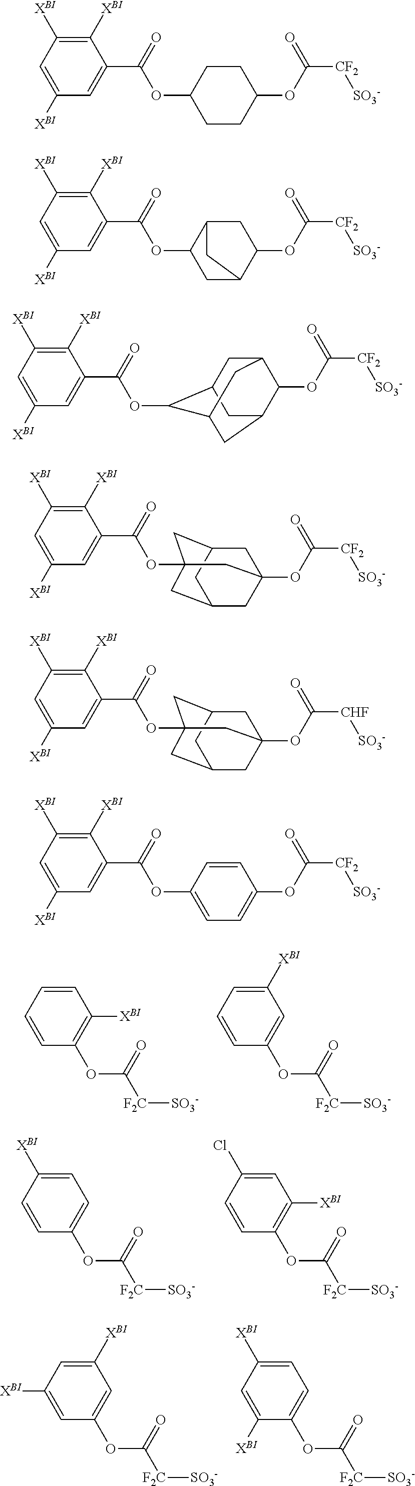

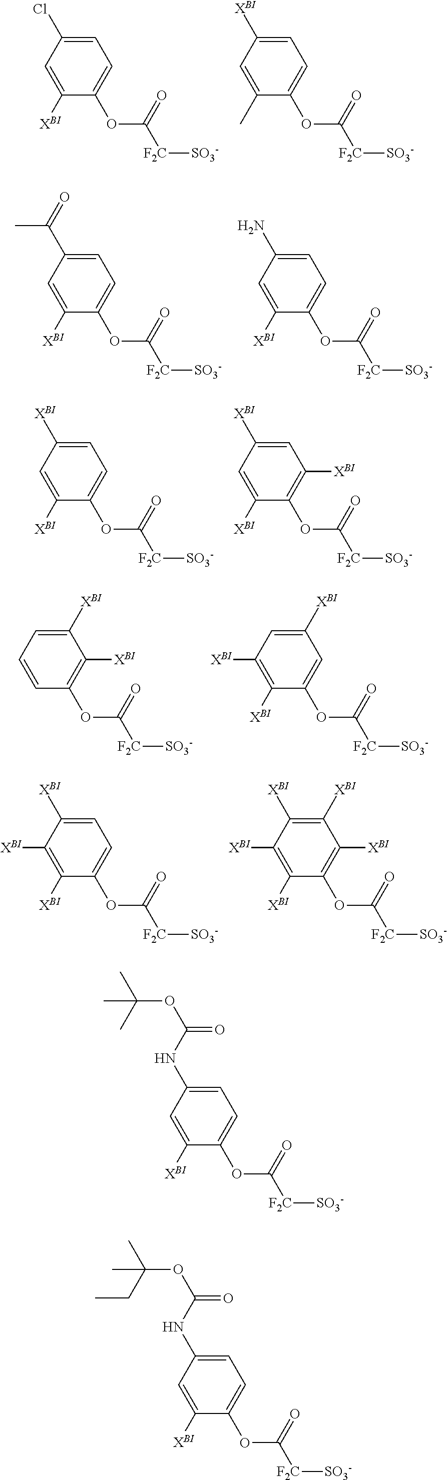

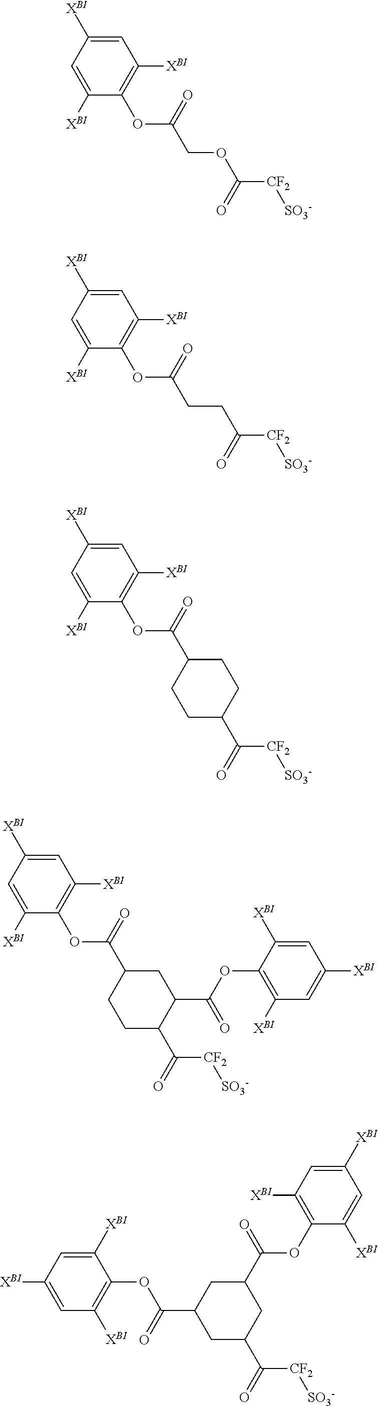

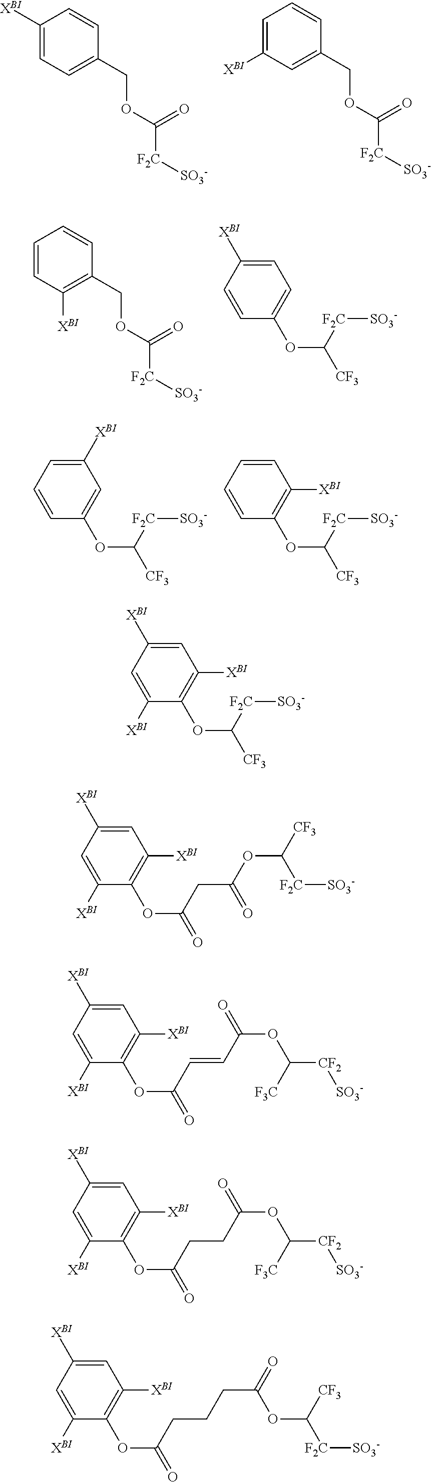

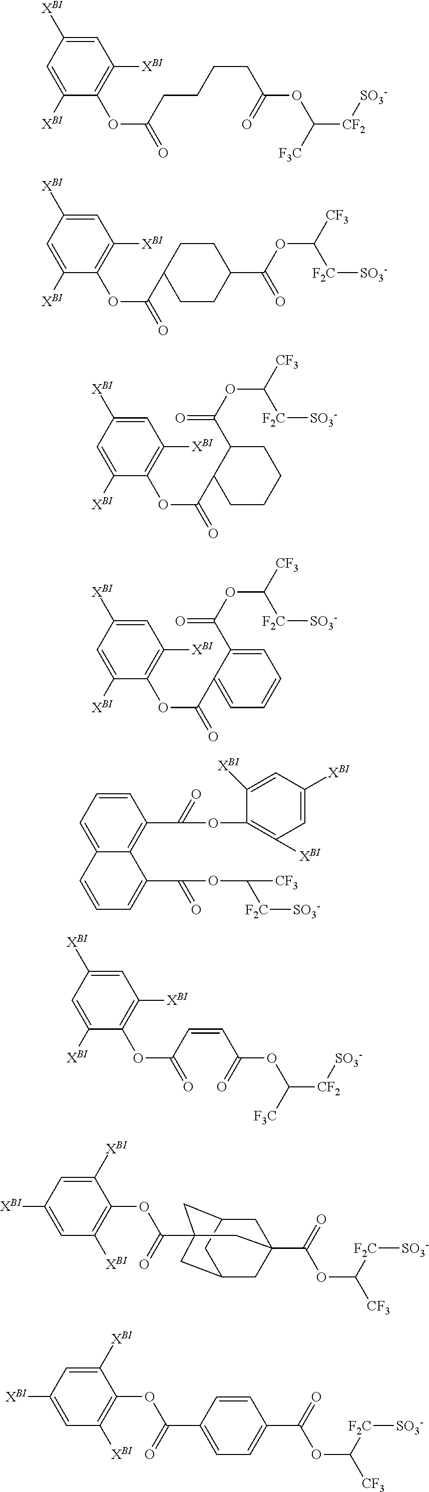

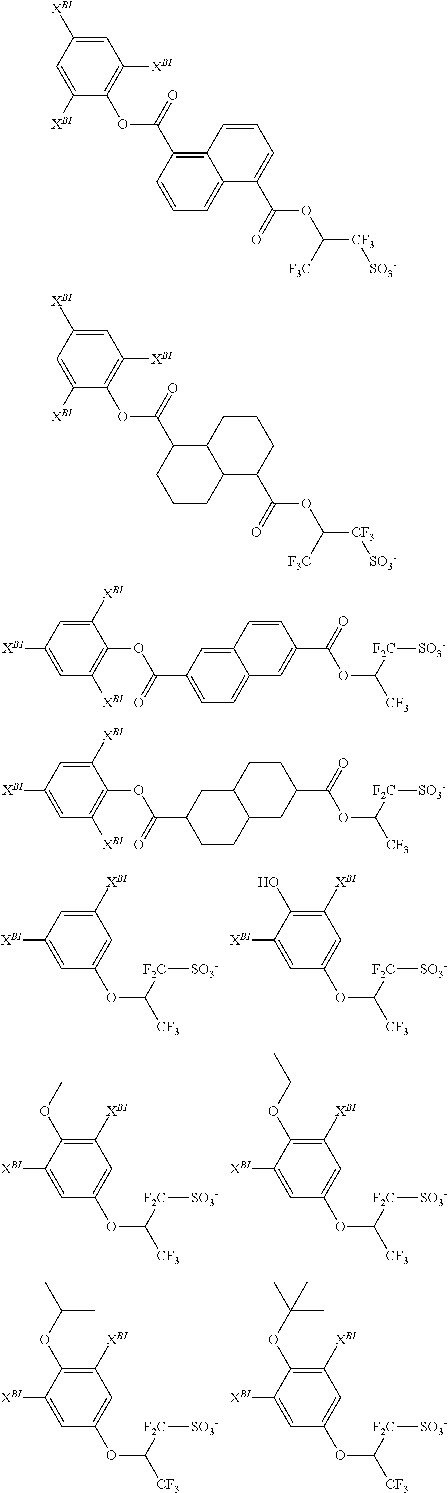

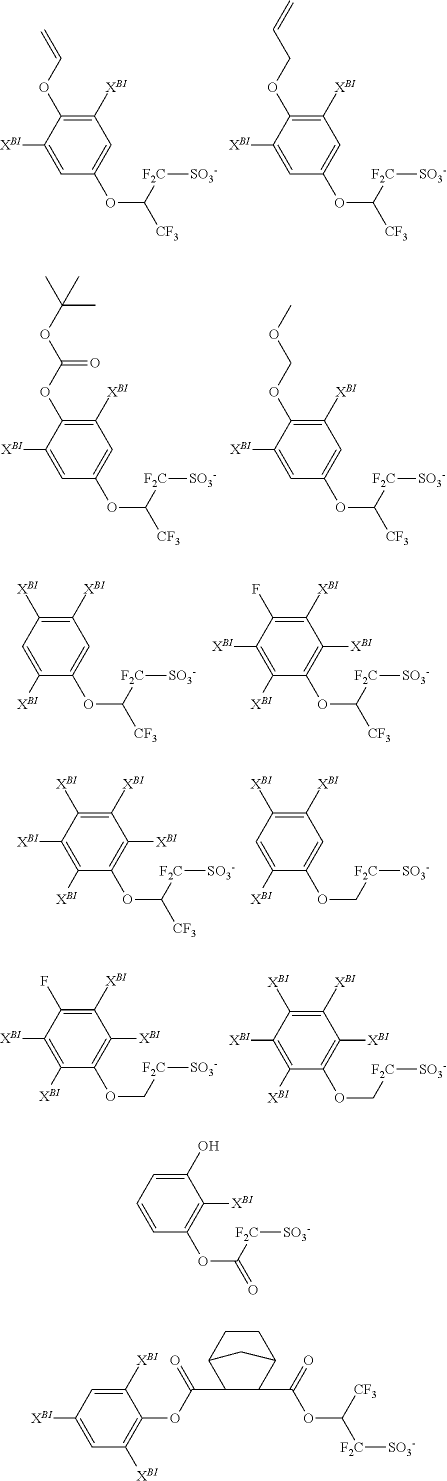

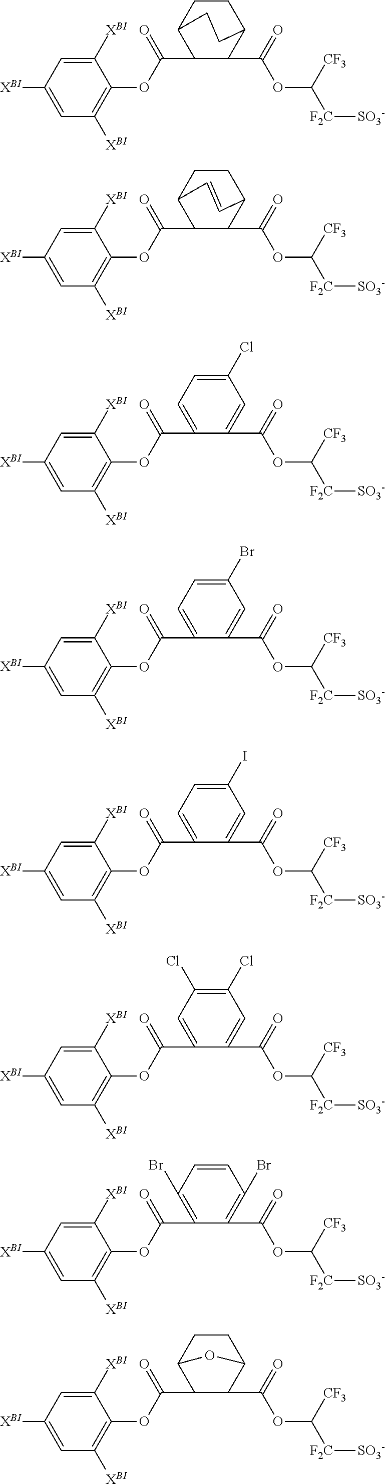

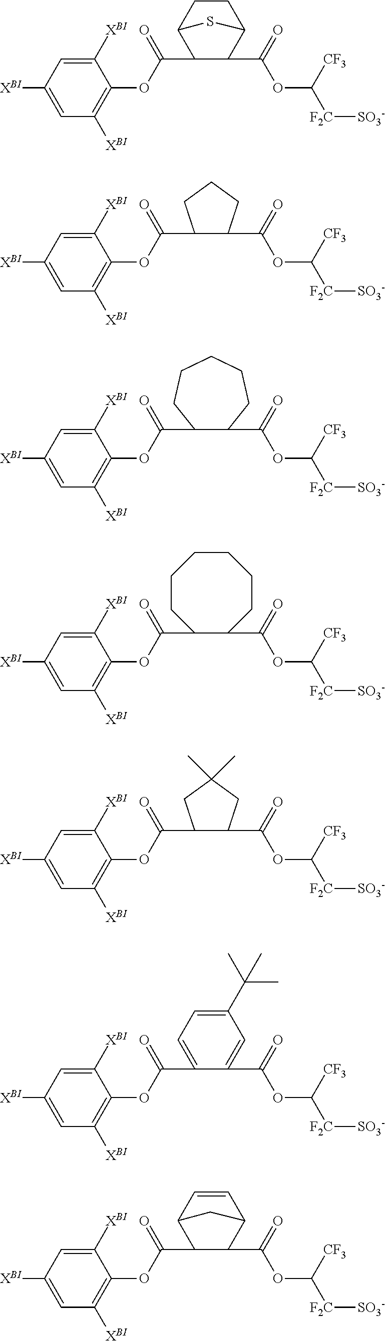

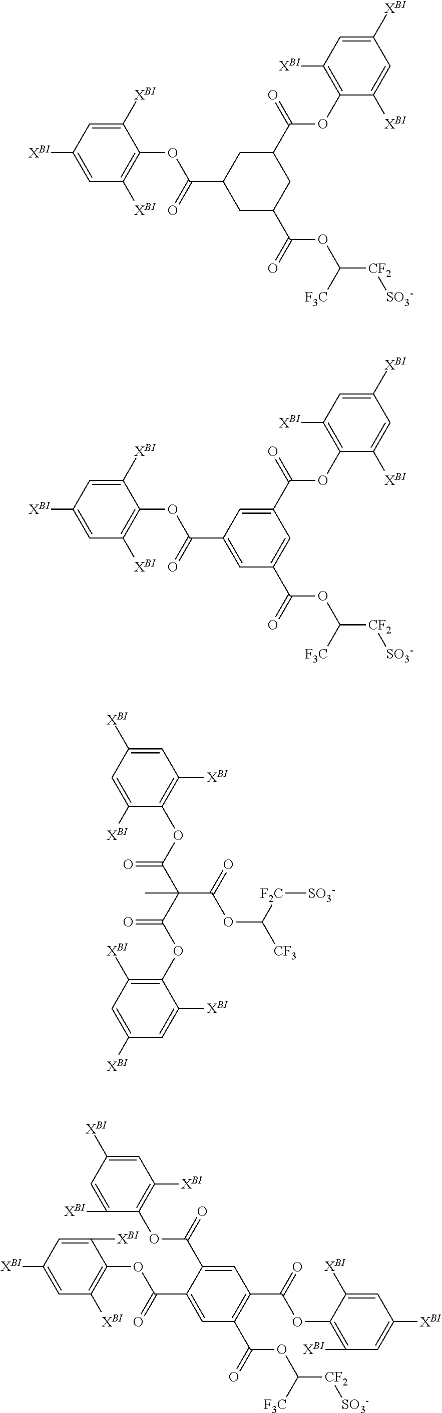

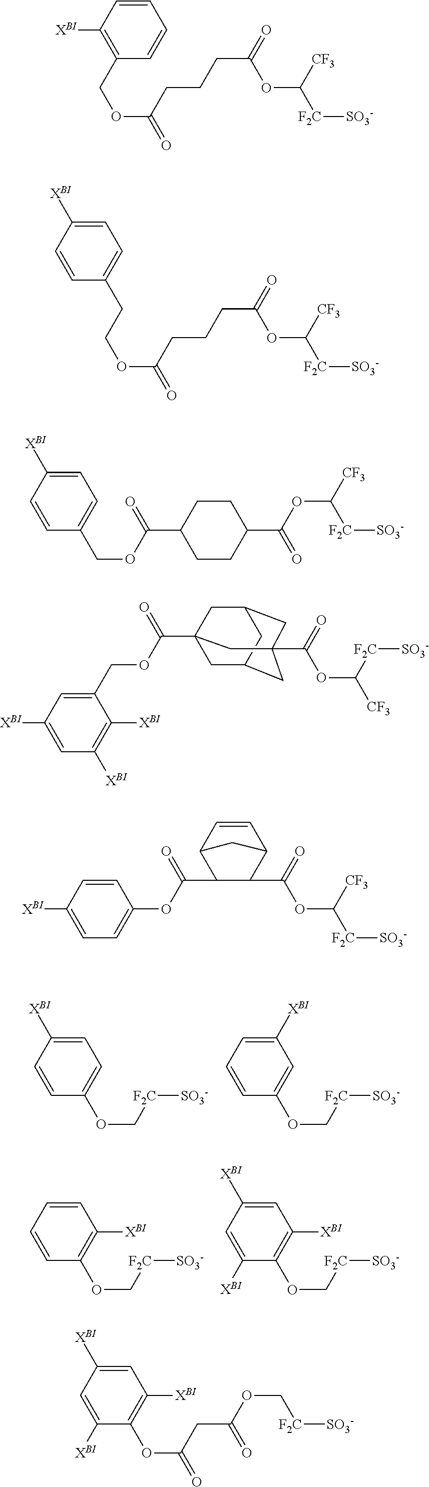

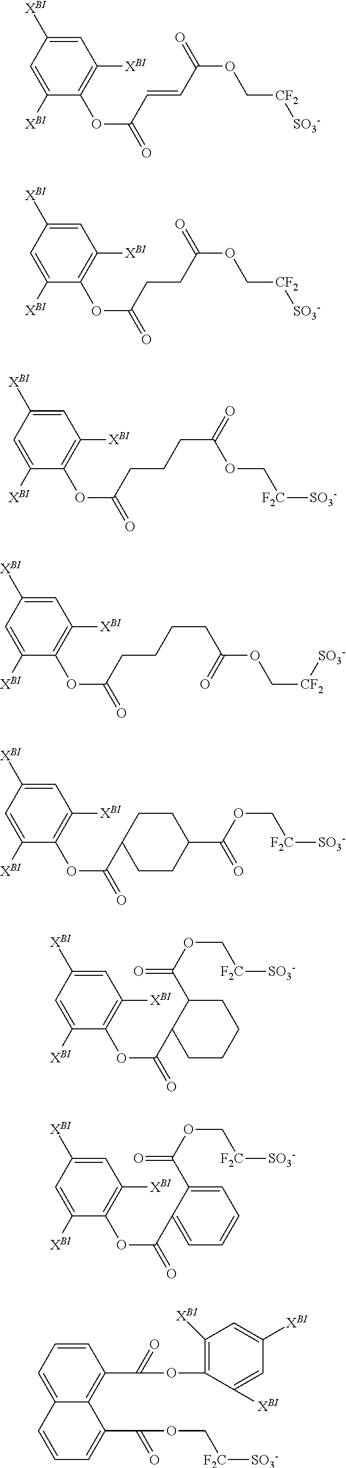

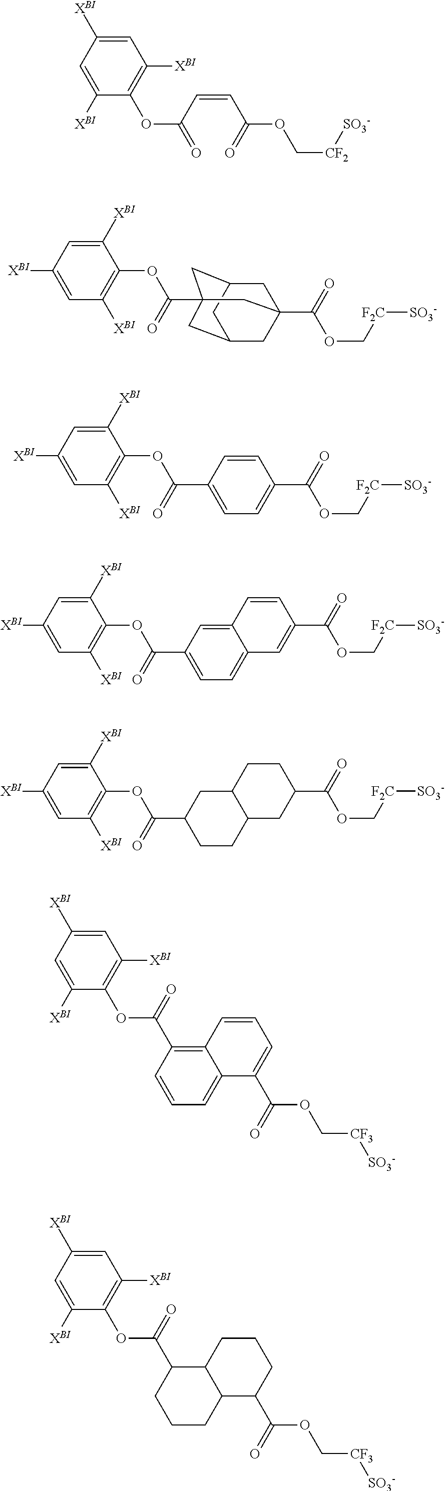

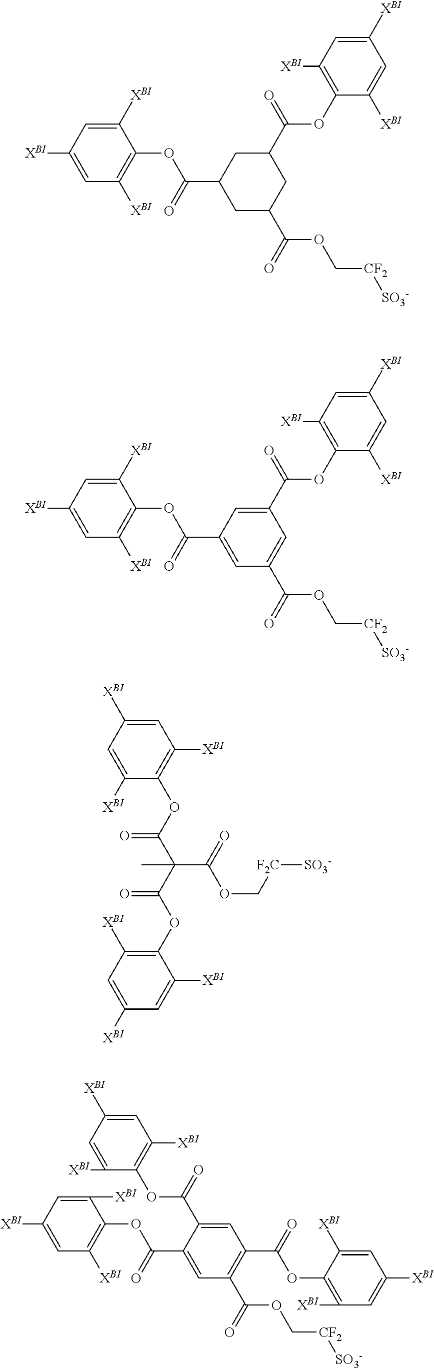

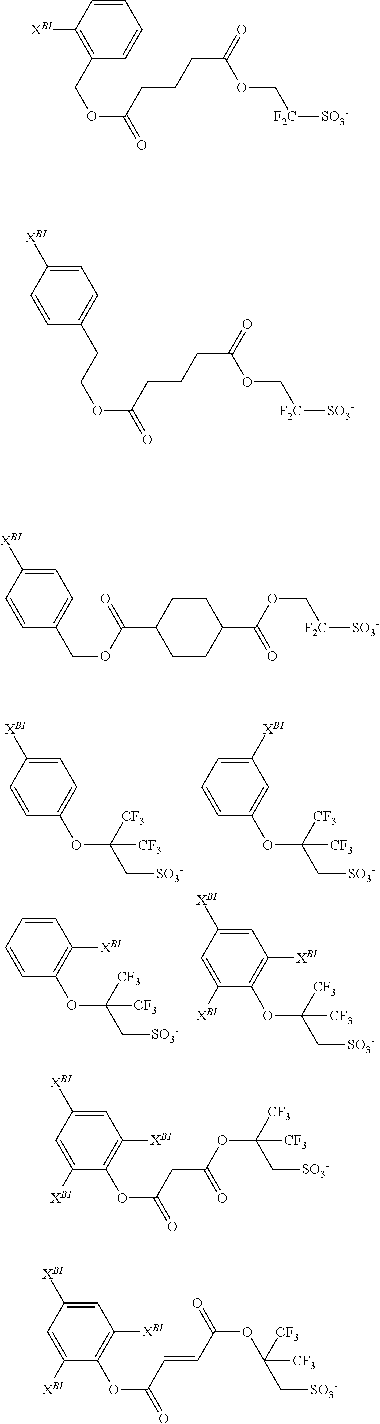

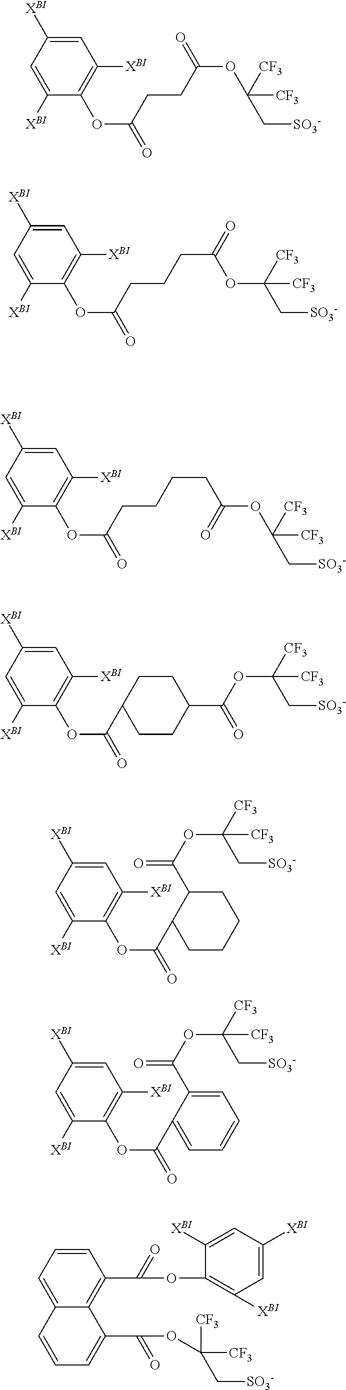

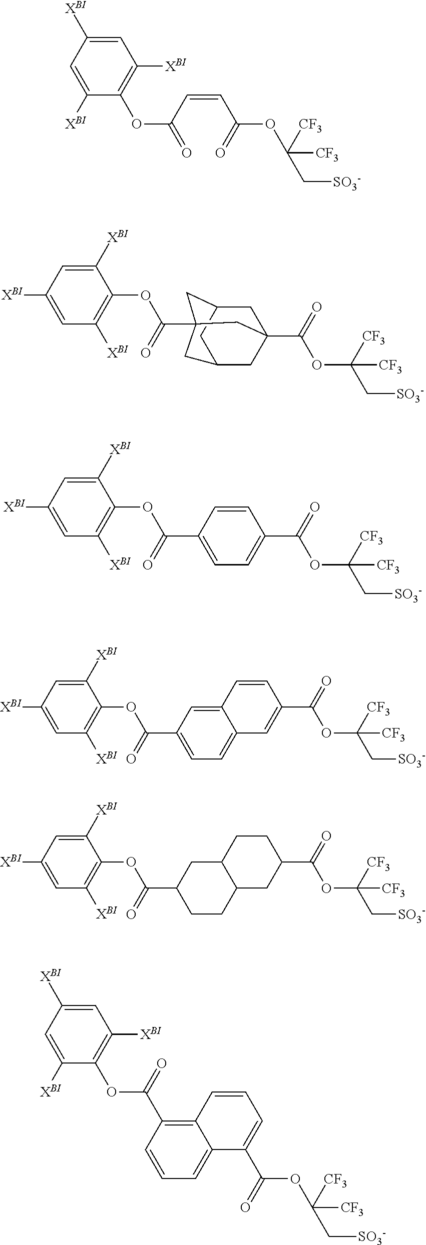

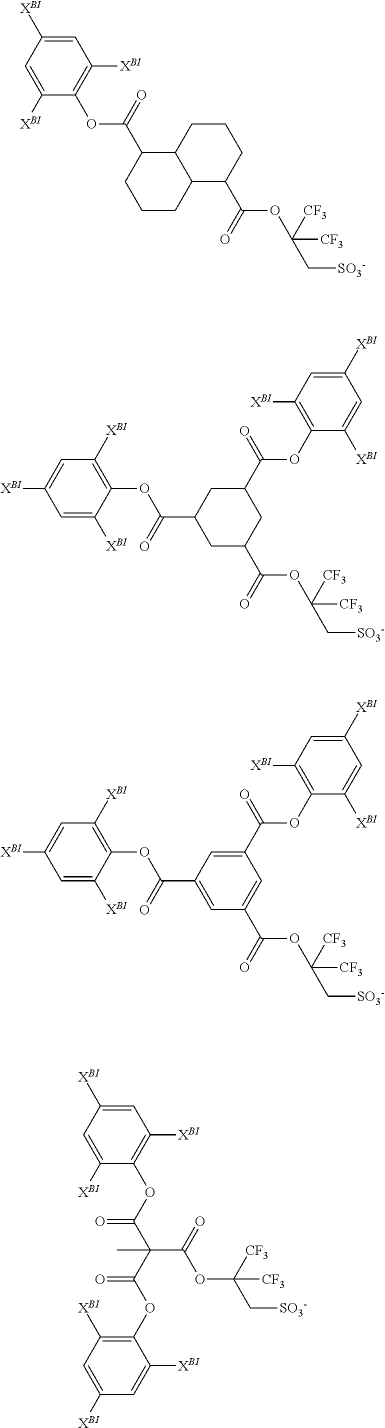

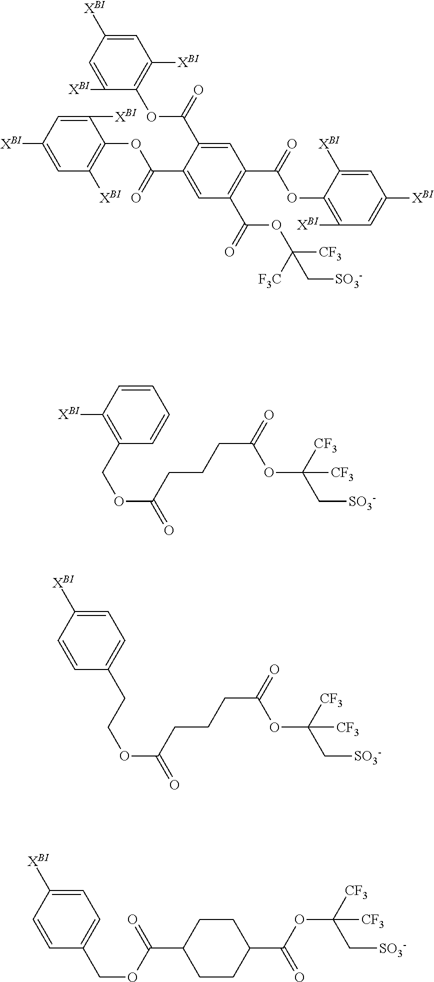

The non-nucleophilic counter ion to be used may include a fluorobenzenesulfonate anion bonded to an aromatic group containing an iodine atom as described in JP 6648726 B; an anion having an acid-catalyzed decomposition mechanism as described in WO 2021/200056 A1 and JP 2021-70692 A; an anion having a cyclic ether group as described in JP 2018-180525 A and JP 2021-35935 A; or an anion as described in JP 2018-92159 A.

The non-nucleophilic counter ion to be used also may include an anion of a bulky benzenesulfonic acid derivative containing no fluorine atom, as described in JP 2006-276759 A, JP 2015-117200 A, JP 2016-65016 A, and JP 2019-202974 A; or a fluorine-free benzenesulfonate anion or alkyl sulfonate anion bonded to an aromatic group containing an iodine atom, as described in JP 6645464 B.

Furthermore, the non-nucleophilic counter ion to be used may include an anion of a bis-sulfonic acid as described in JP 2015-206932 A; an anion having a sulfonic acid on one side and a sulfonamide or sulfonimide different therefrom on the other side, as described in WO 2020/158366 A1; or an anion having a sulfonic acid on one side and a carboxylic acid on the other side, as described in JP 2015-24989 A.

In formulae (c2) and (c3), “d1” and “d2” are each independently 0, 1, 2, or 3, preferably 1.

In formula (c4), “e1” is 0 or 1. “e2” is 0, 1, 2, 3, or 4, and “e3” is 0, 1, 2, 3, or 4, provided that when “e1” is 0, 0≤e2+e3≤4, and when “e1” is 1, 0≤e2+e3≤6.

In formulae (c2), (c3), and (c4), L¹ is a single bond, an ether bond, an ester bond, a carbonyl group, a sulfonate ester bond, a sulfonamide bond, a carbonate bond, or a carbamate bond. Of these, from the viewpoint of synthesis, an ether bond, an ester bond, and a carbonyl group are preferable, and an ester bond and a carbonyl group are further preferable.

In formula (c2), Rf¹ and Rf² are each independently a fluorine atom or a fluorinated saturated hydrocarbyl group having 1 to 6 carbon atoms. Of these, both Rf¹ and Rf² are preferably fluorine atoms in order to increase the acid strength of the generated acid. Rf³ and Rf⁴ are each independently a hydrogen atom, a fluorine atom, or a fluorinated saturated hydrocarbyl group having 1 to 6 carbon atoms. Of these, at least one of the Rf³ and Rf⁴ is preferably a trifluoromethyl group in order to enhance solvent solubility.

In formula (c3), Rf⁵ and Rf⁶ are each independently a hydrogen atom, a fluorine atom, or a fluorinated saturated hydrocarbyl group having 1 to 6 carbon atoms, provided that not all Rf⁵ and Rf⁶ are hydrogen atoms at the same time. Of these, at least one of the Rf⁵ and Rf⁶ is preferably a trifluoromethyl group in order to enhance solvent solubility.

In formula (c4), RF⁷ is a fluorine atom, a fluorinated alkyl group having 1 to 6 carbon atoms, a fluorinated alkoxy group having 1 to 6 carbon atoms, or a fluorinated alkylthio group having 1 to 6 carbon atoms. Rf⁷ is preferably a fluorine atom, a trifluoromethyl group, a difluoromethyl group, a trifluoromethoxy group, a difluoromethoxy group, a trifluoromethylthio group, or a difluoromethylthio group, and more preferably a fluorine atom, a trifluoromethyl group, or a trifluoromethoxy group. When “f” is 2, 3, or 4, Rf⁷ are the same as or different from each other.

In formula (c4), R³³ is a halogen atom other than a fluorine atom or a hydrocarbyl group having 1 to 20 carbon atoms and optionally comprising a heteroatom. The hydrocarbyl group may be either saturated or unsaturated and may be linear, branched, or cyclic. Specific examples thereof include, but are not limited to, the same hydrocarbyl group represented by R¹ in the description of formula (1). When “e3” is 2, 3, or 4, R³³ are the same as or different from each other.

In addition, when “e3” is 2, 3, or 4, a plurality of R³³ are optionally bonded to each other to form a ring together with carbon atoms to which the R³³ are bonded. Specific examples of the ring formed at this time include a cyclopropane ring, a cyclobutane ring, a cyclopentane ring, a cyclohexane ring, a norbornane ring, and an adamantane ring. Some or all of the hydrogen atoms in the ring are optionally substituted with a group containing a heteroatom such as an oxygen atom, a sulfur atom, a nitrogen atom, or a halogen atom, and some of —CH₂— in the ring are optionally substituted with a group containing a heteroatom such as an oxygen atom, a sulfur atom, or a nitrogen atom. As a result, the ring may include, for instance, a hydroxy group, a fluorine atom, a chlorine atom, a bromine atom, an iodine atom, a cyano group, a carbonyl group, an ether bond, an ester bond, a sulfonate ester bond, a carbonate bond, a lactone ring, a sultone ring, a carboxylic anhydride(—C(═O)—O—C(═O)—), and a haloalkyl group.

Specific examples of the anion in the repeating unit c2 are given below, but not limited thereto. In formulae below, R^{A} is the same as above, and Me represents a methyl group.

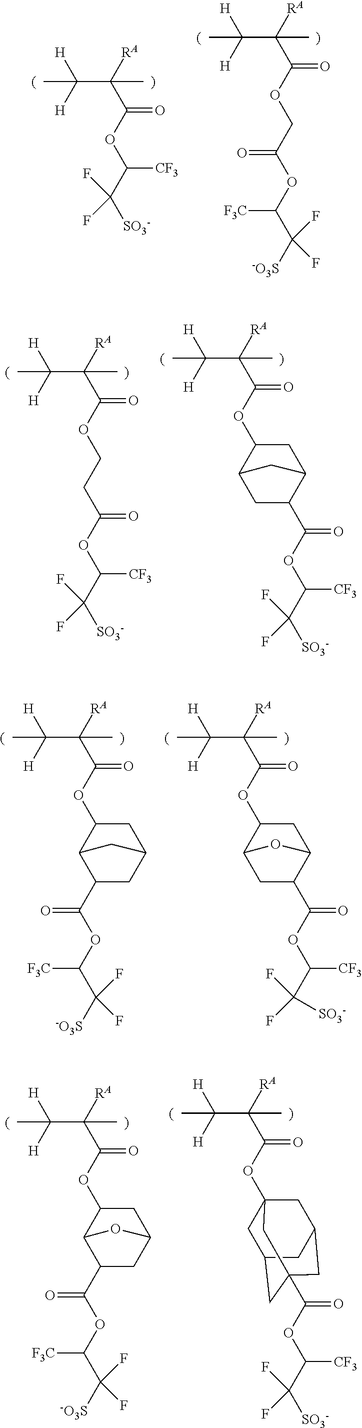

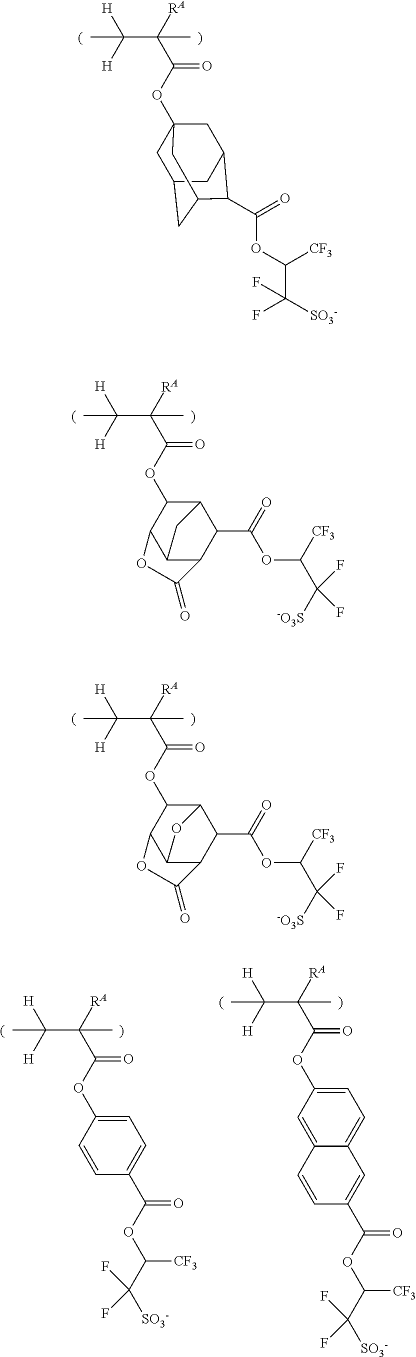

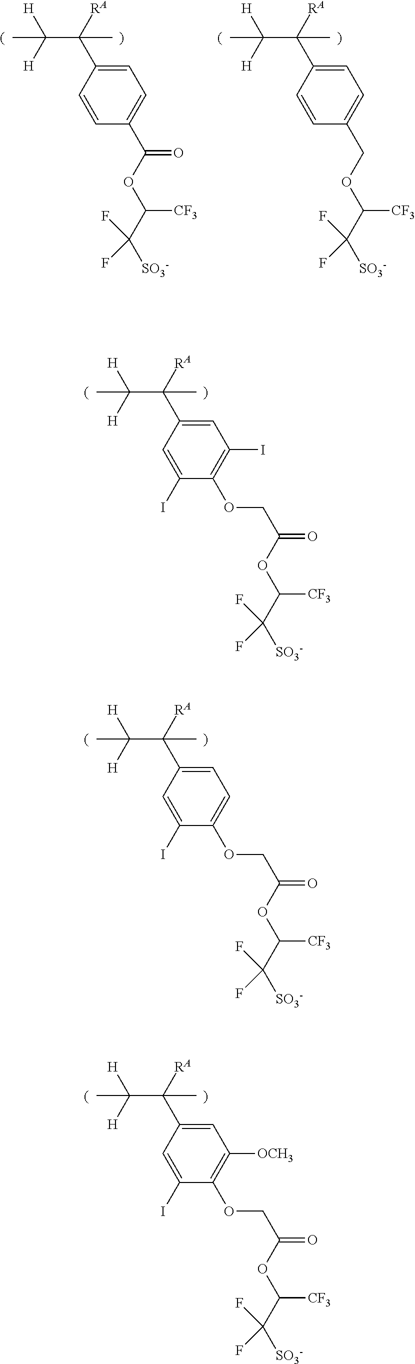

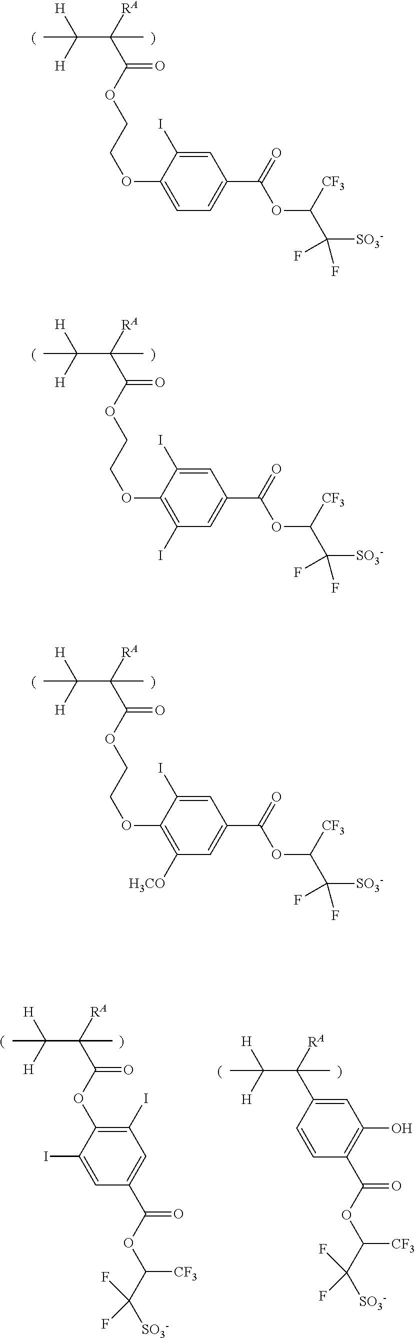

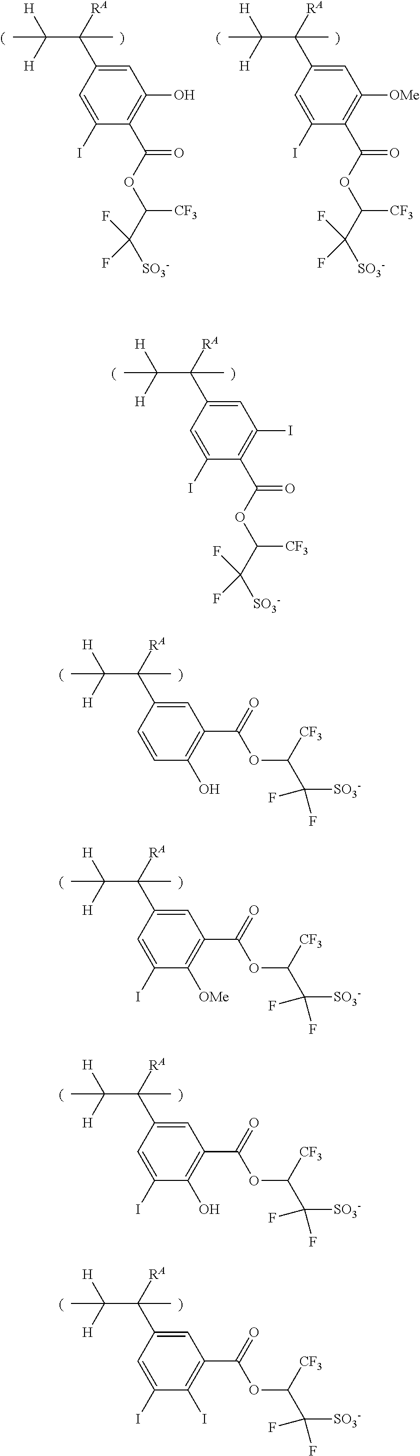

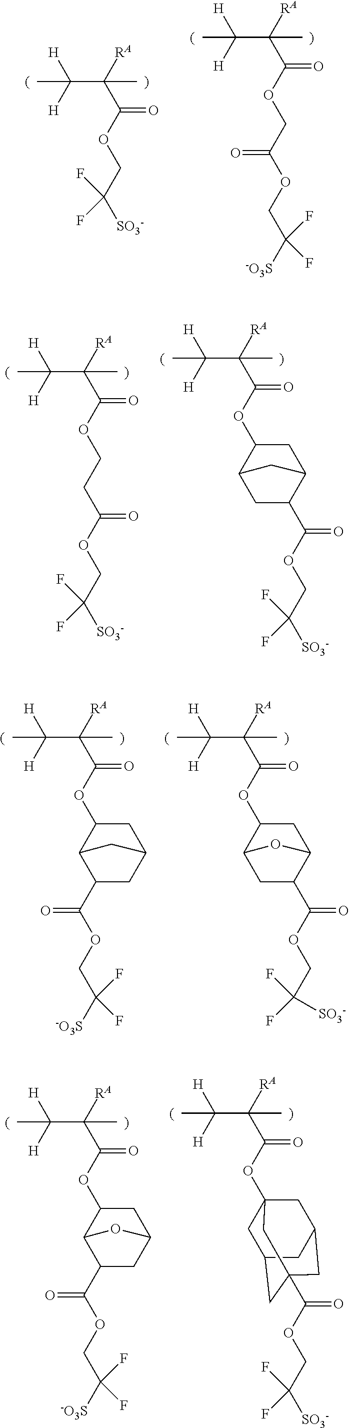

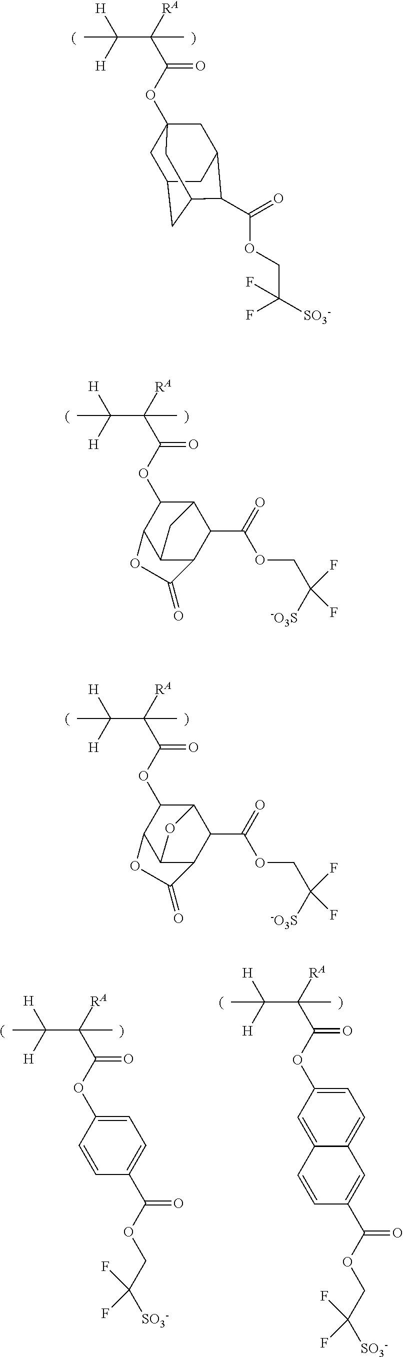

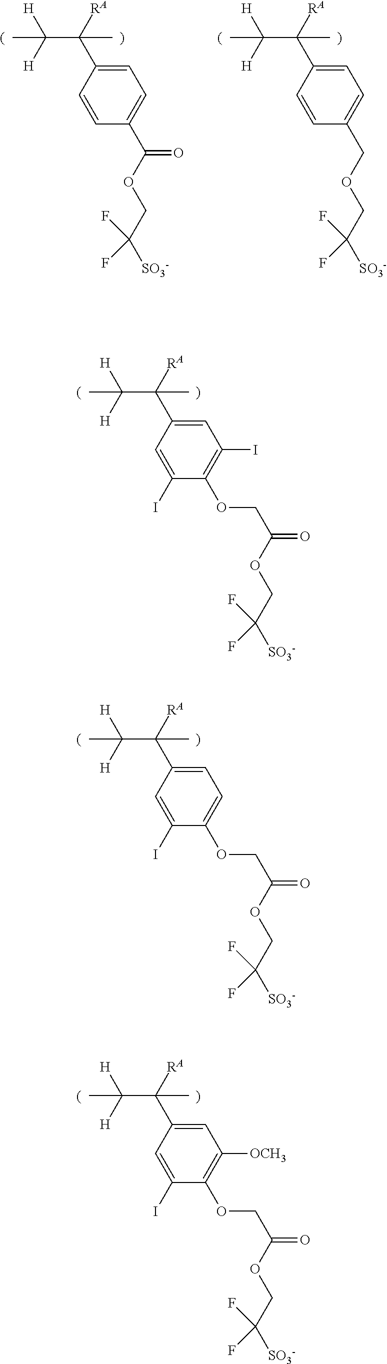

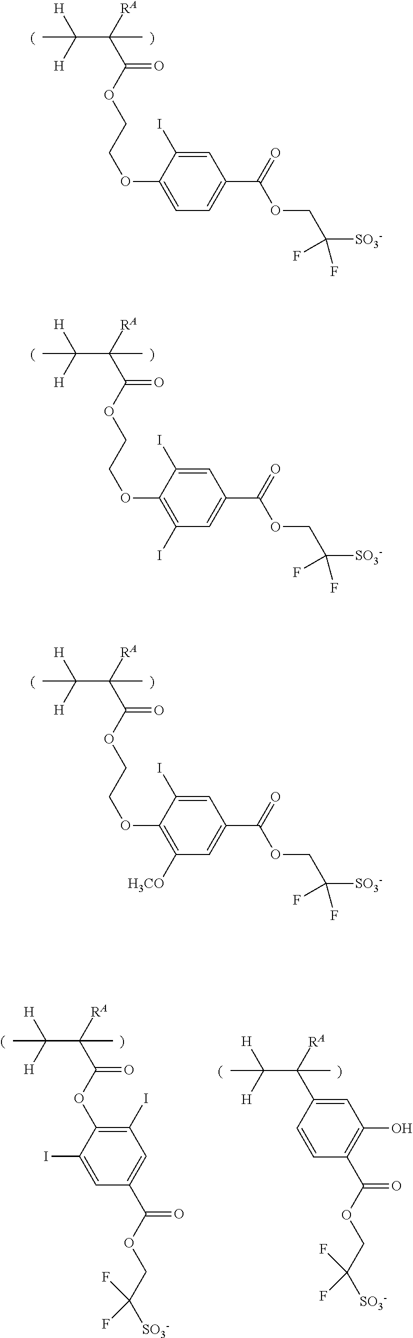

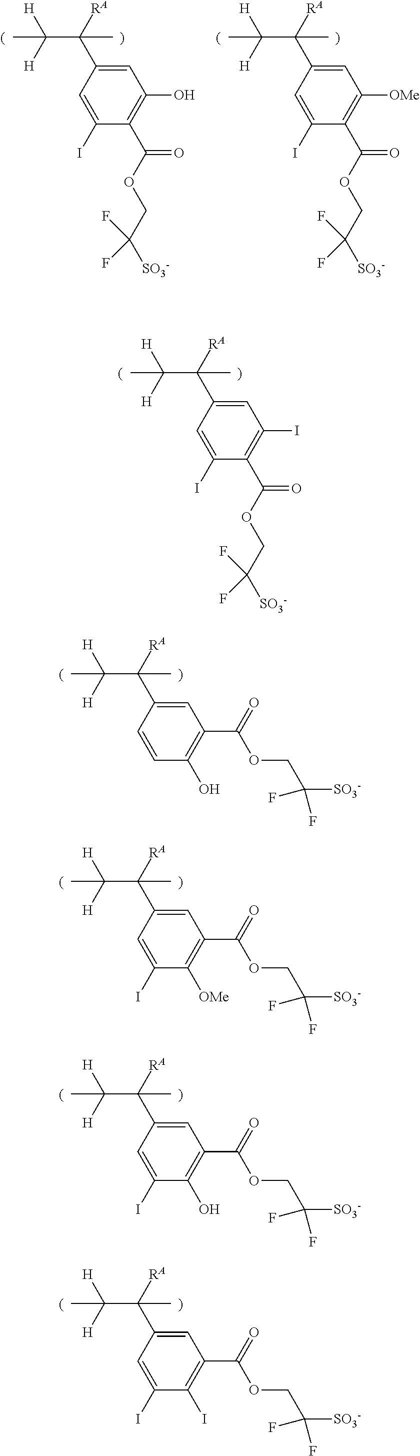

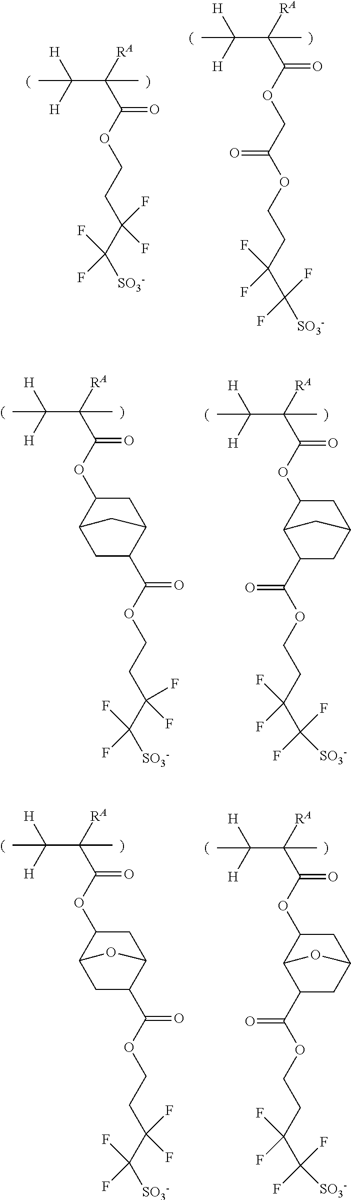

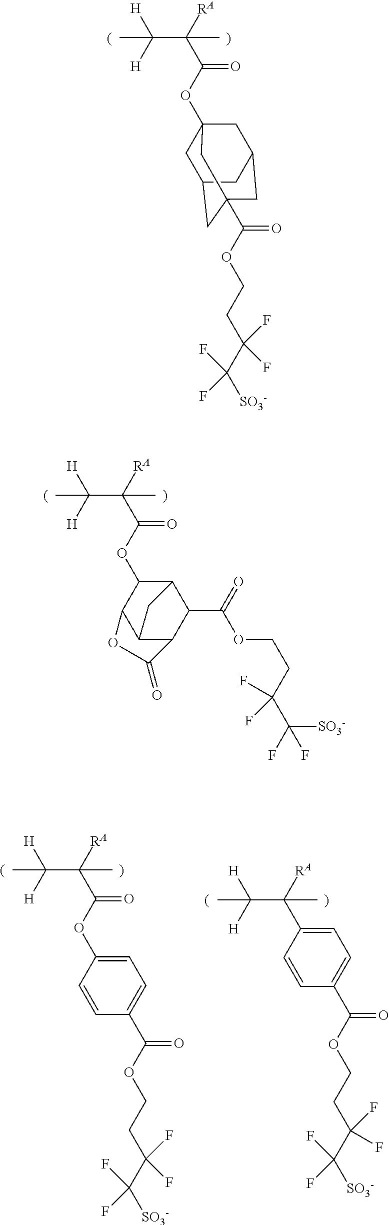

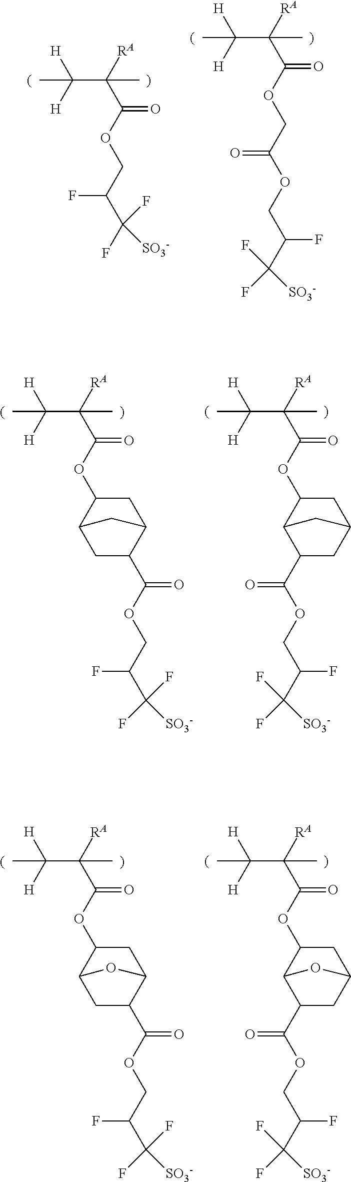

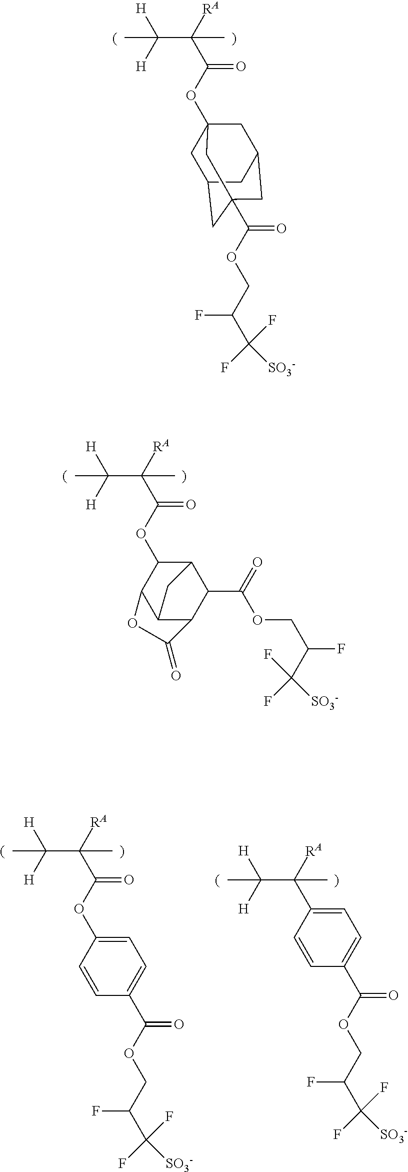

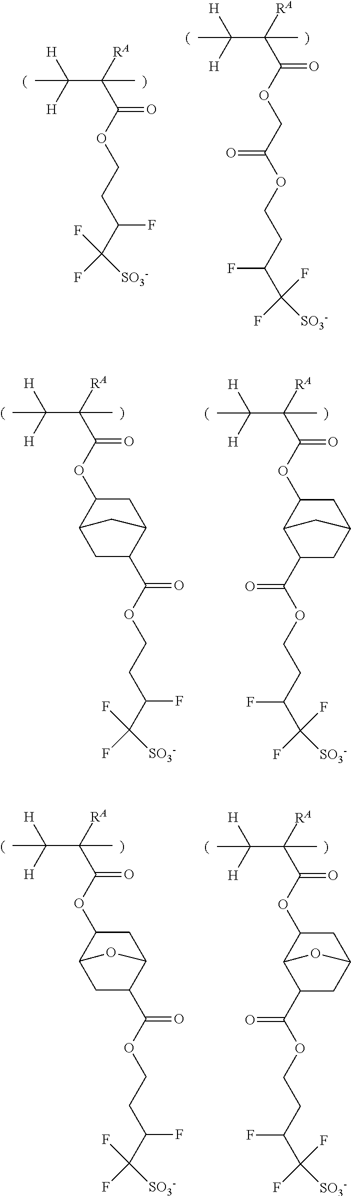

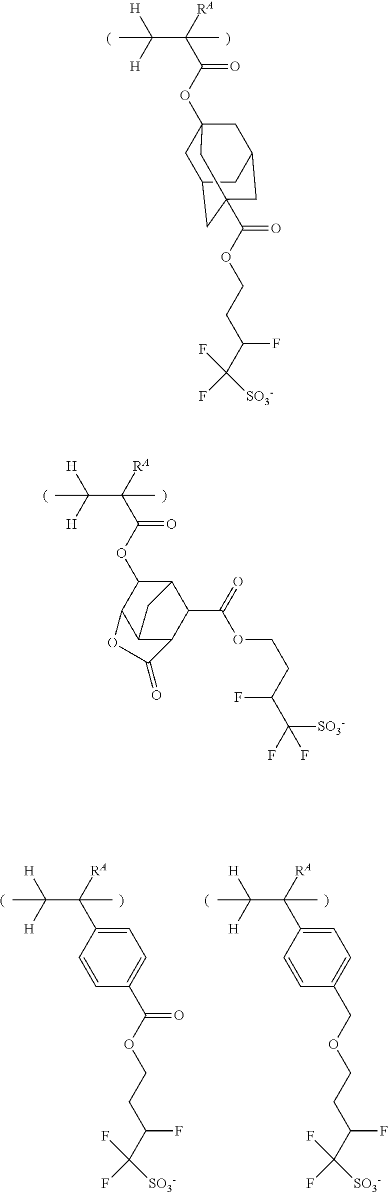

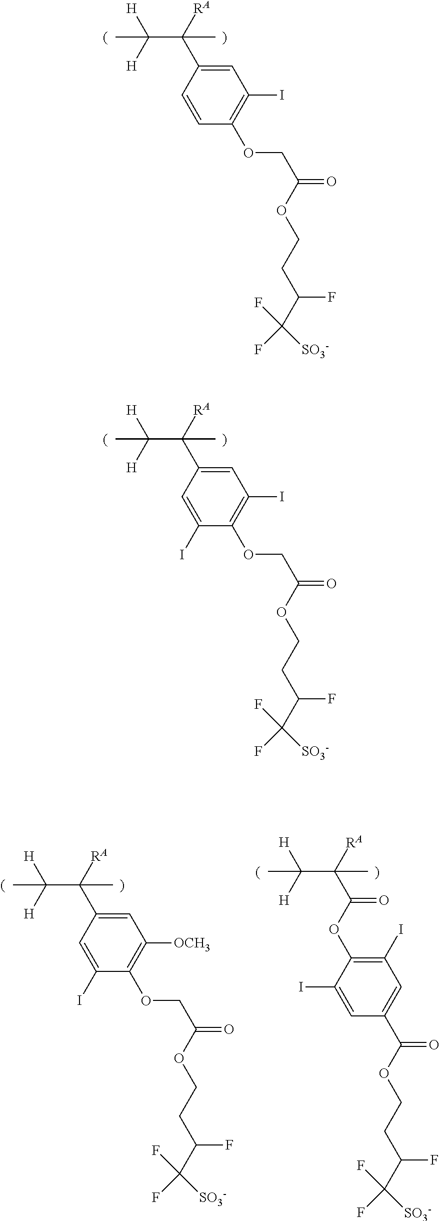

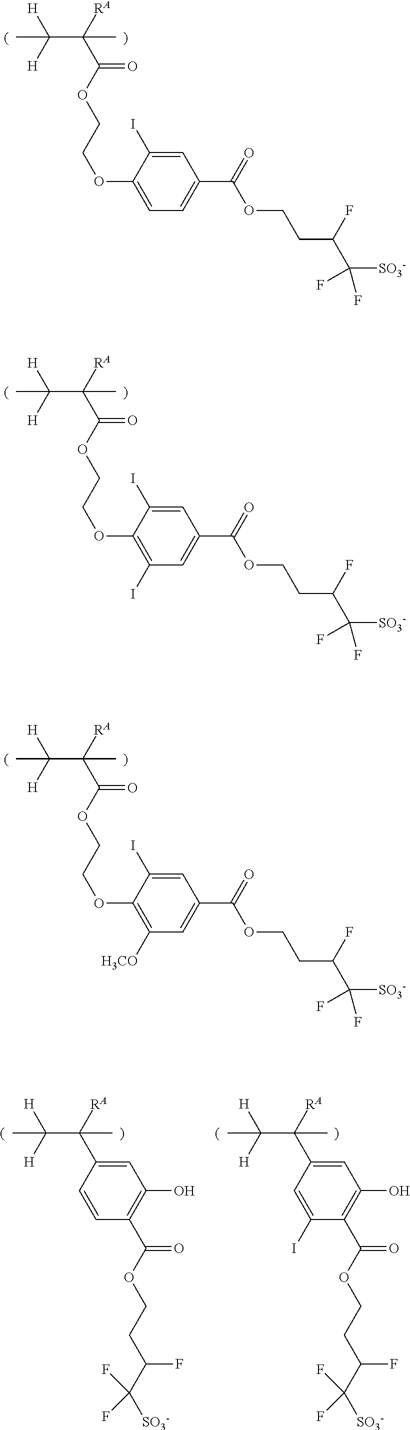

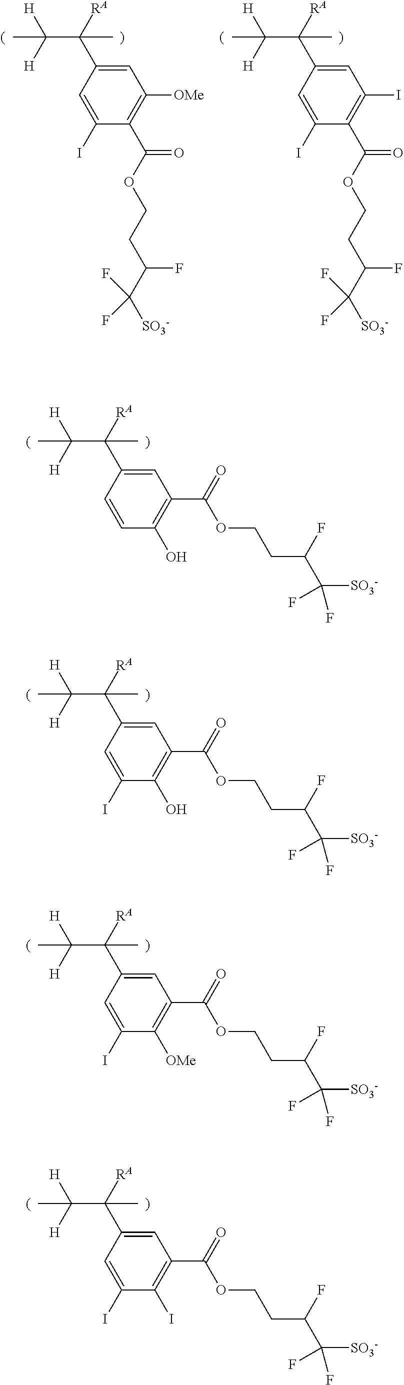

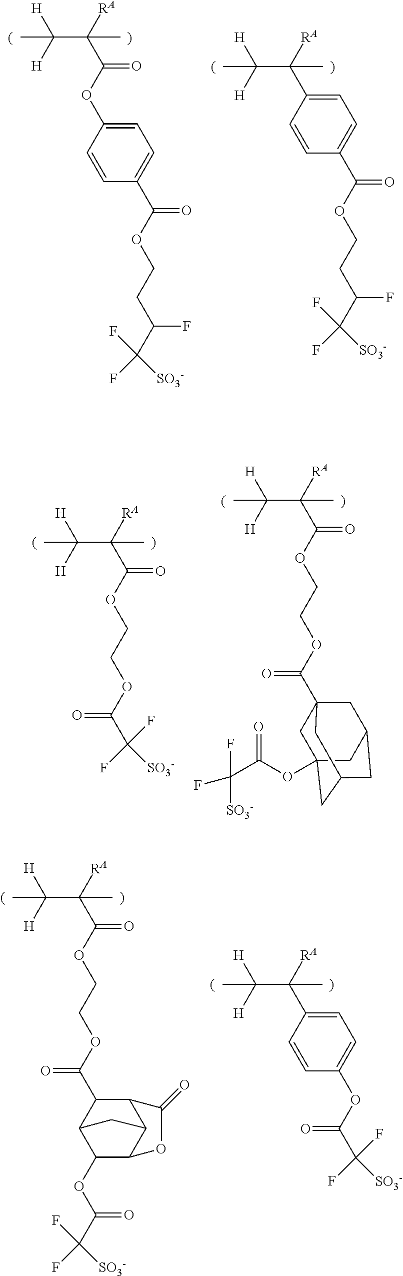

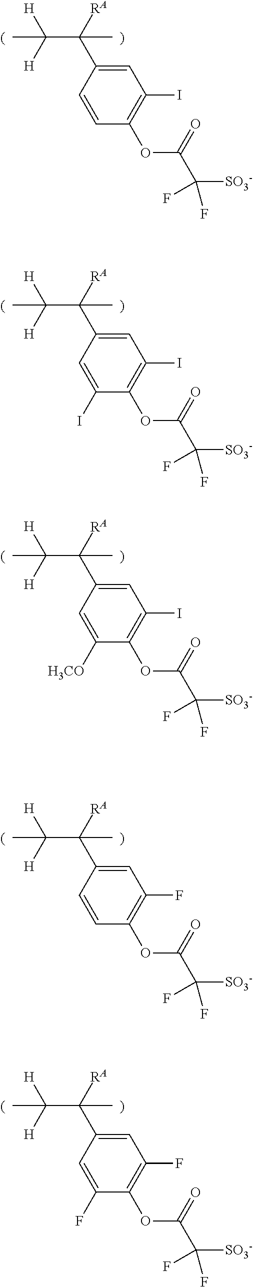

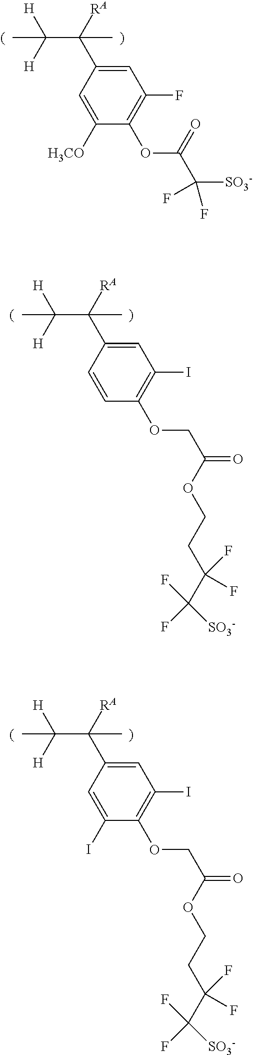

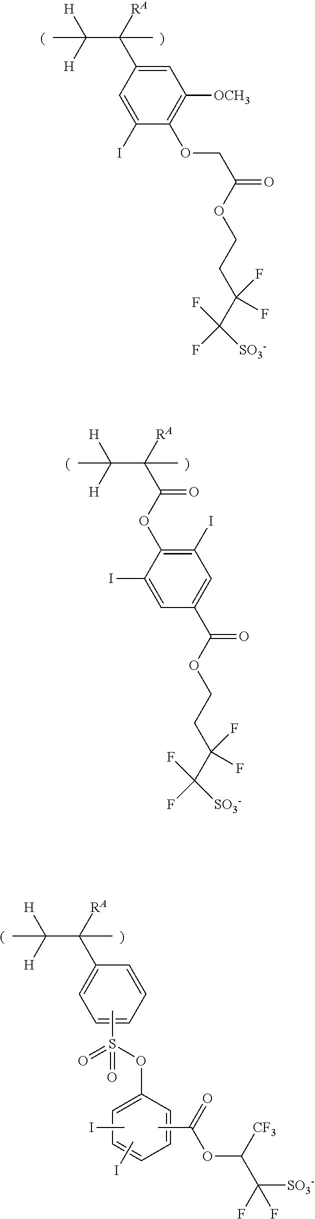

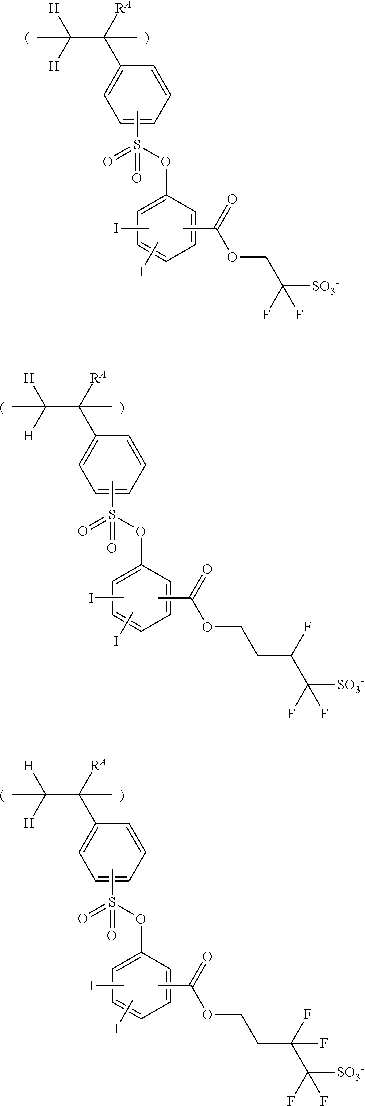

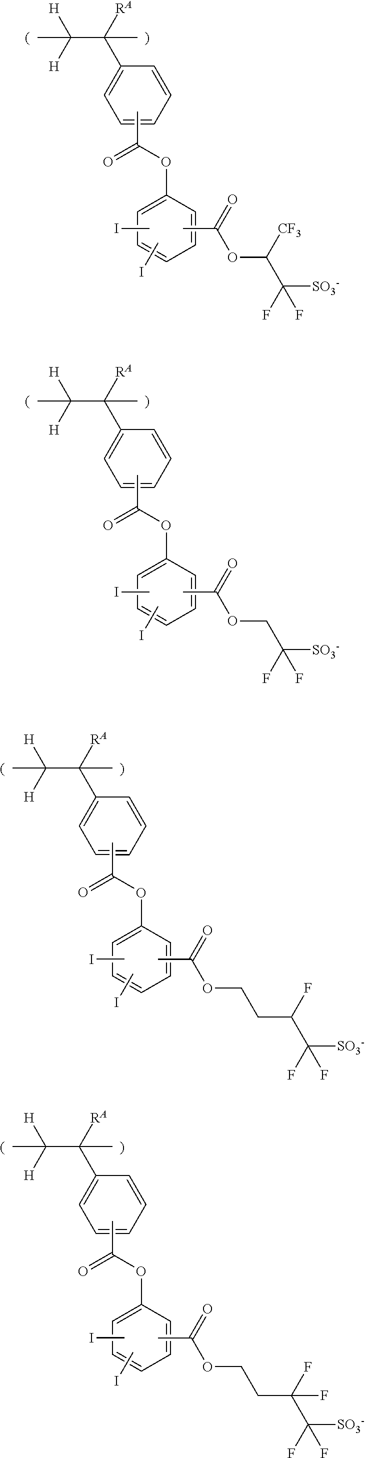

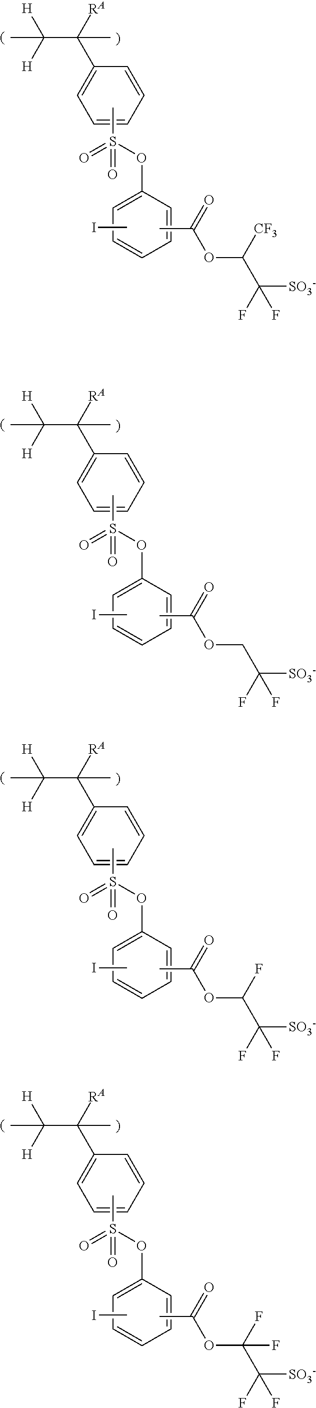

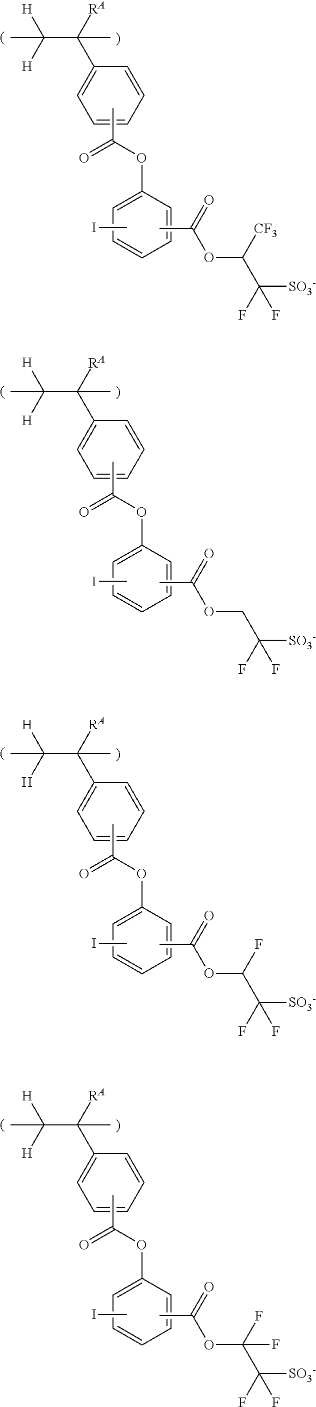

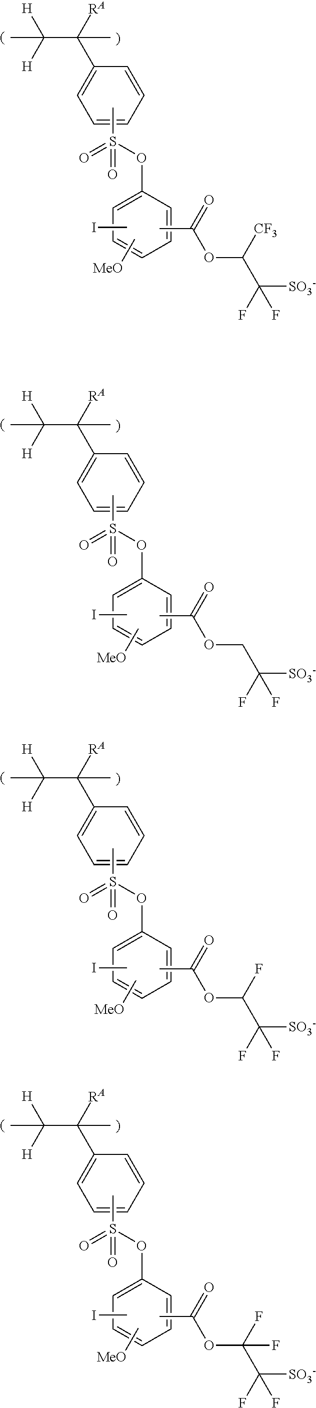

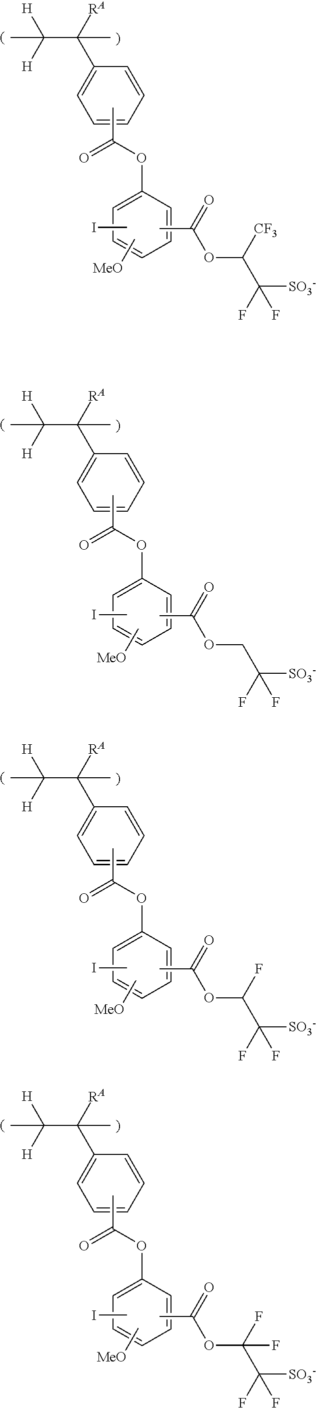

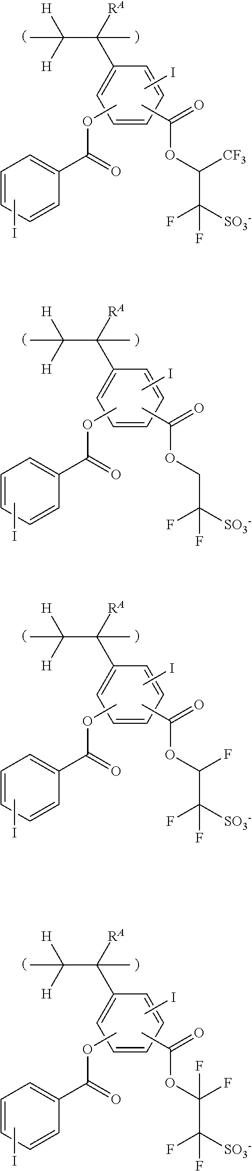

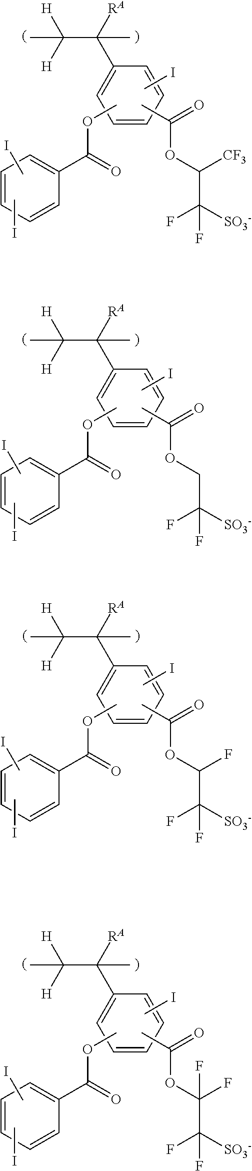

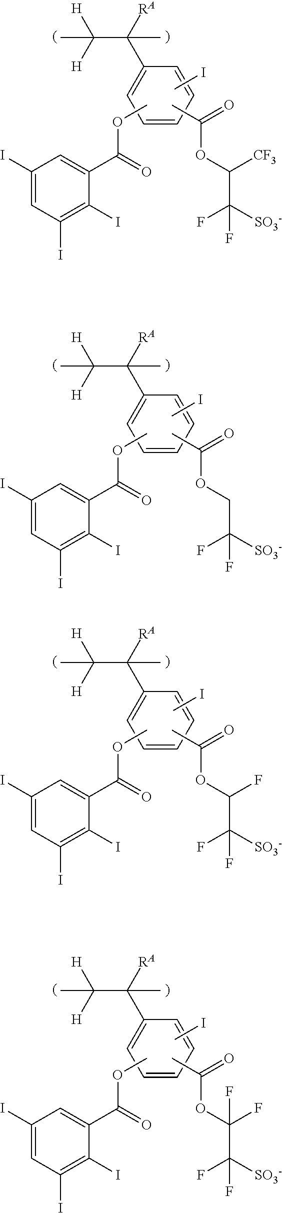

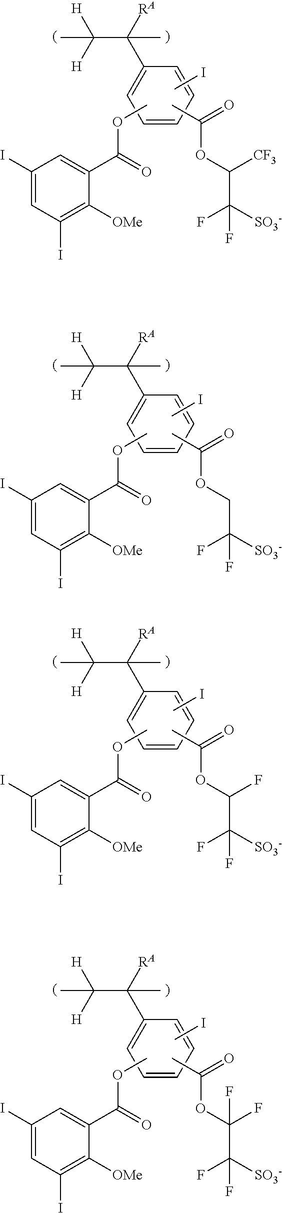

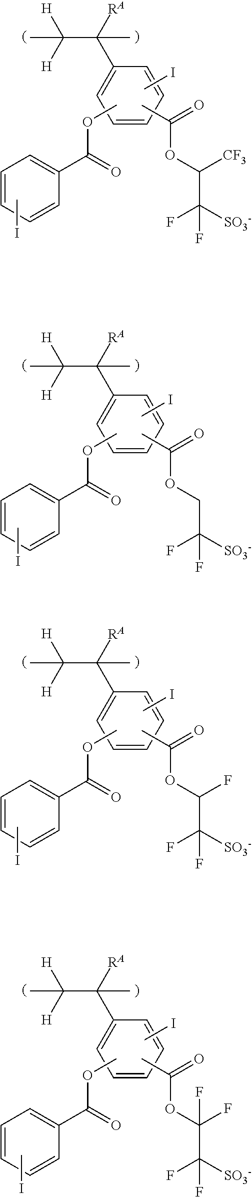

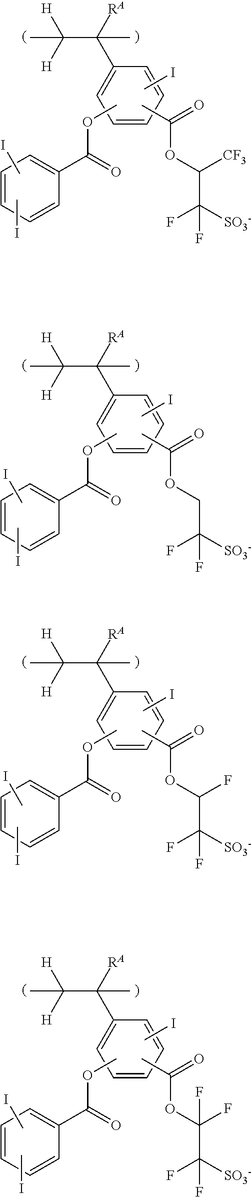

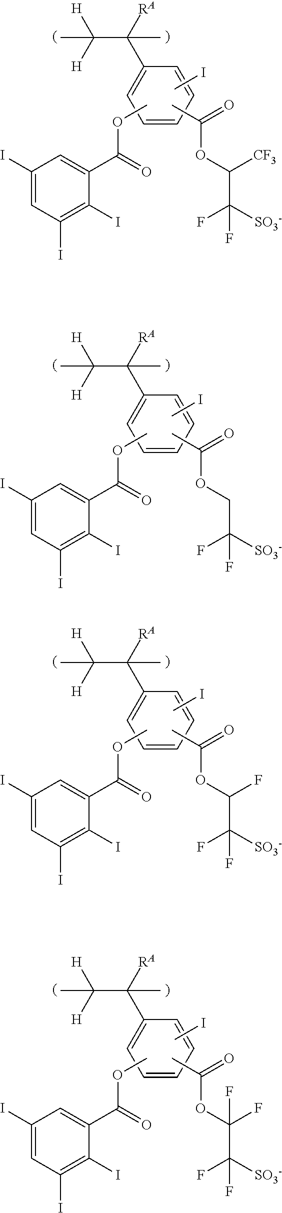

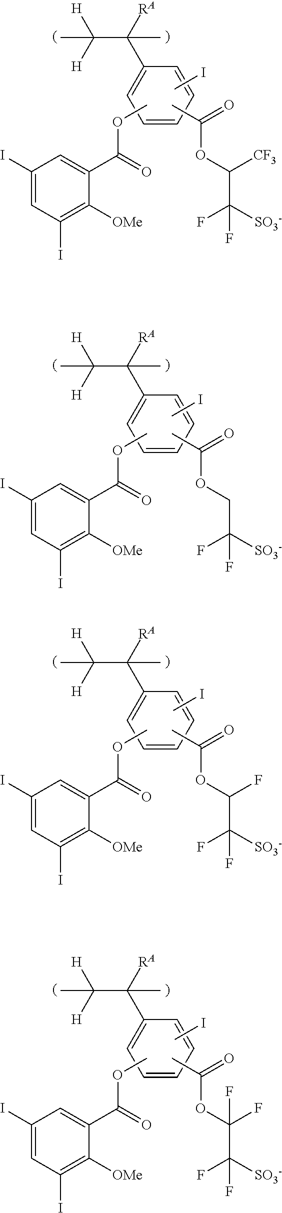

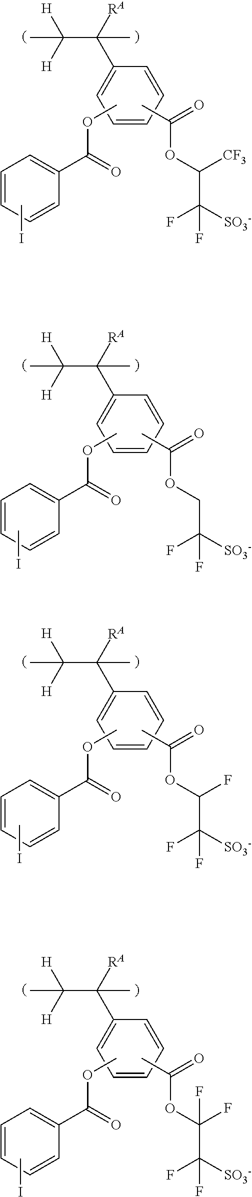

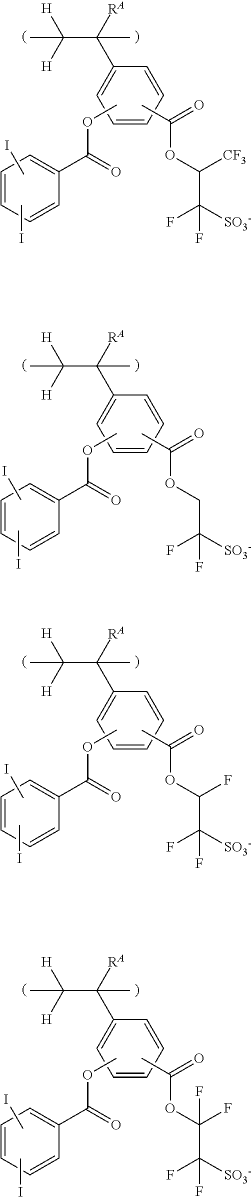

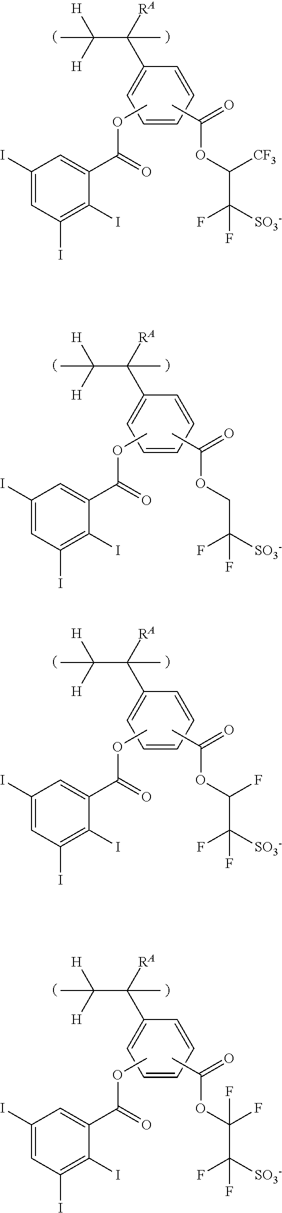

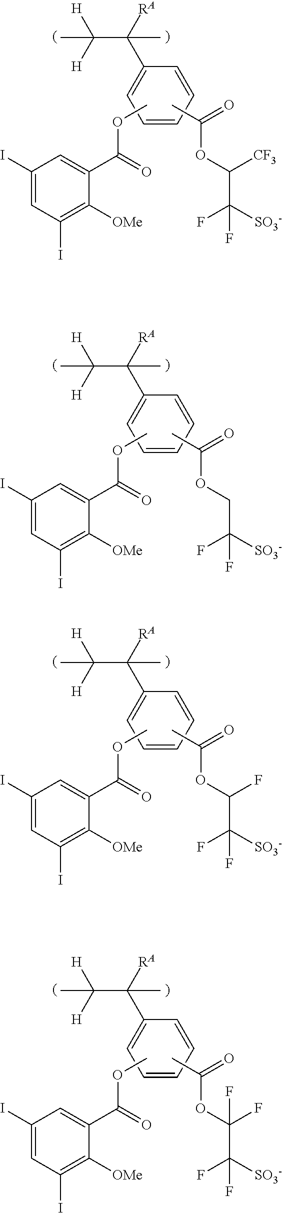

Specific examples of the anion in the repeating unit c3 are given below, but not limited thereto. In the formulae below, R is the same as above.

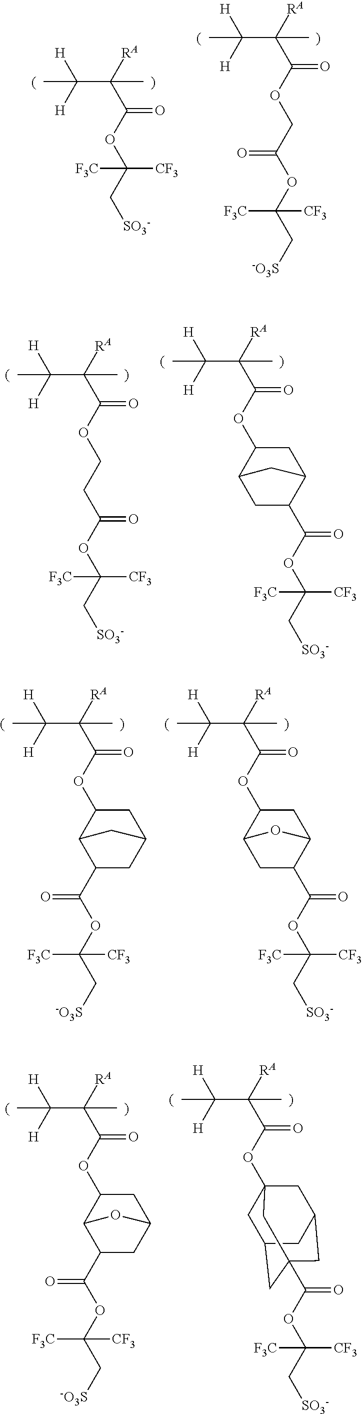

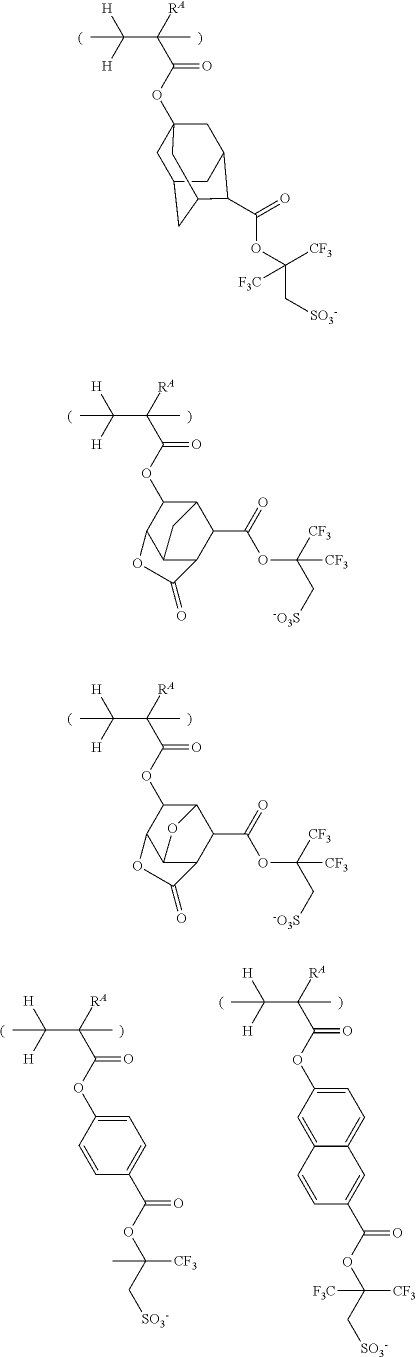

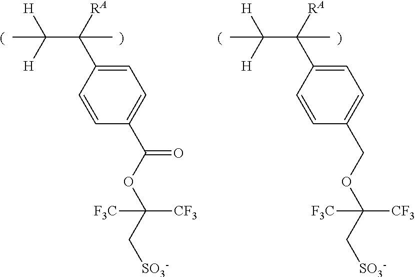

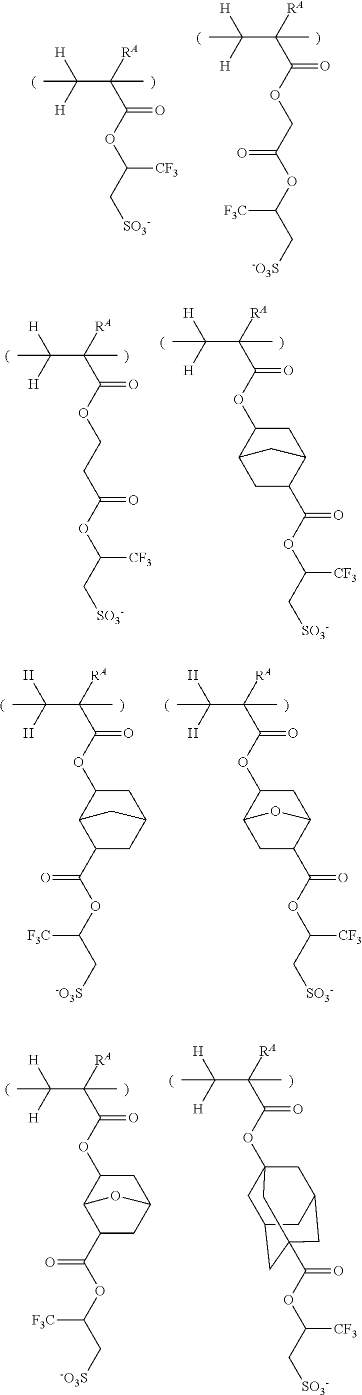

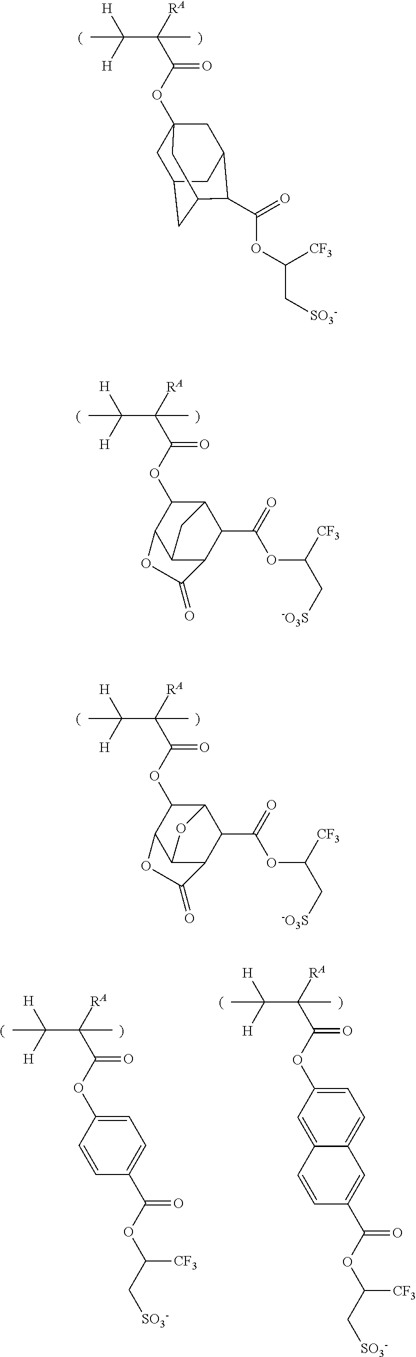

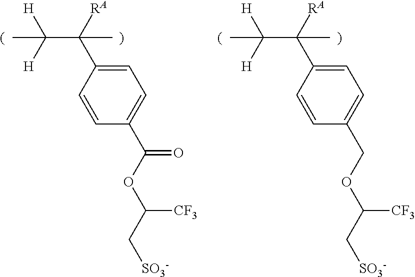

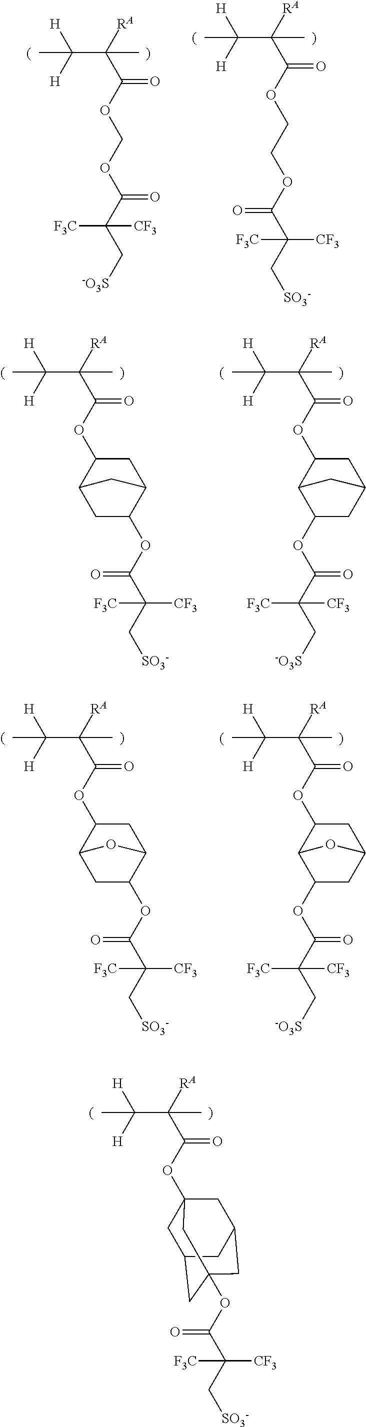

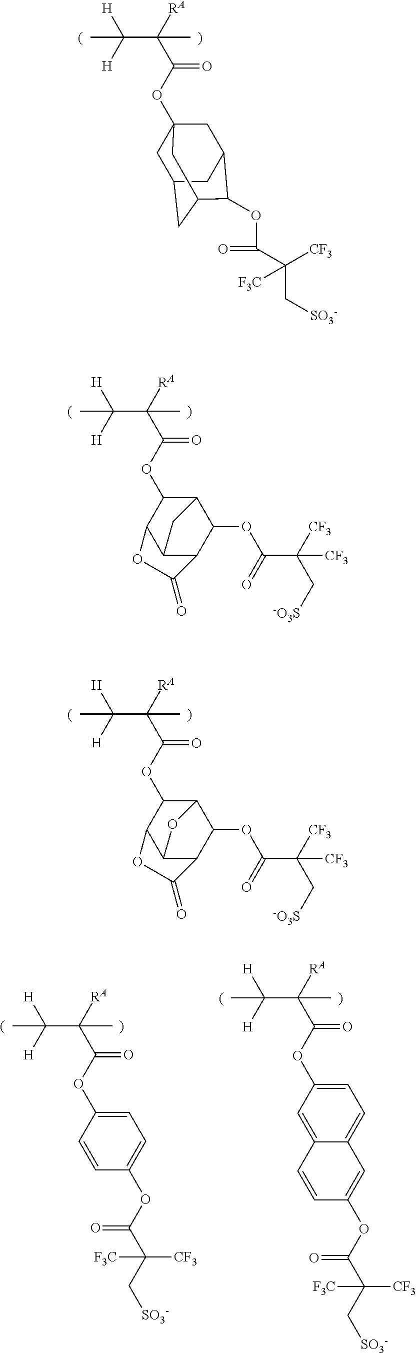

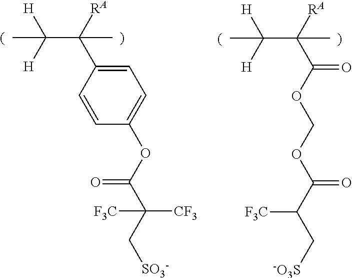

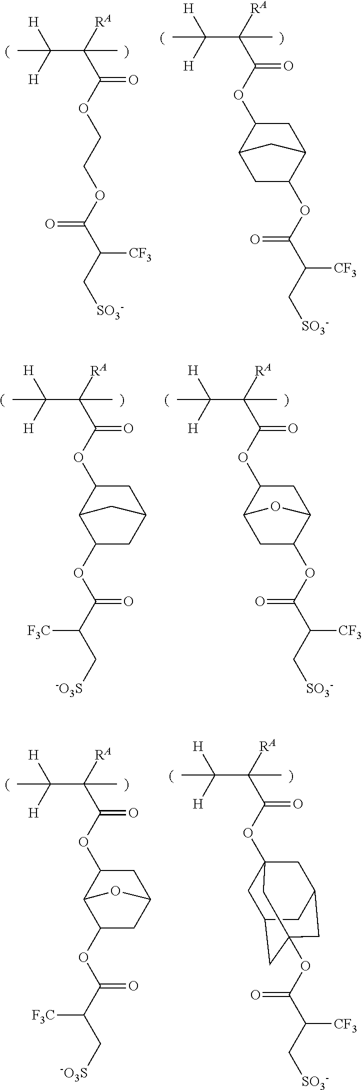

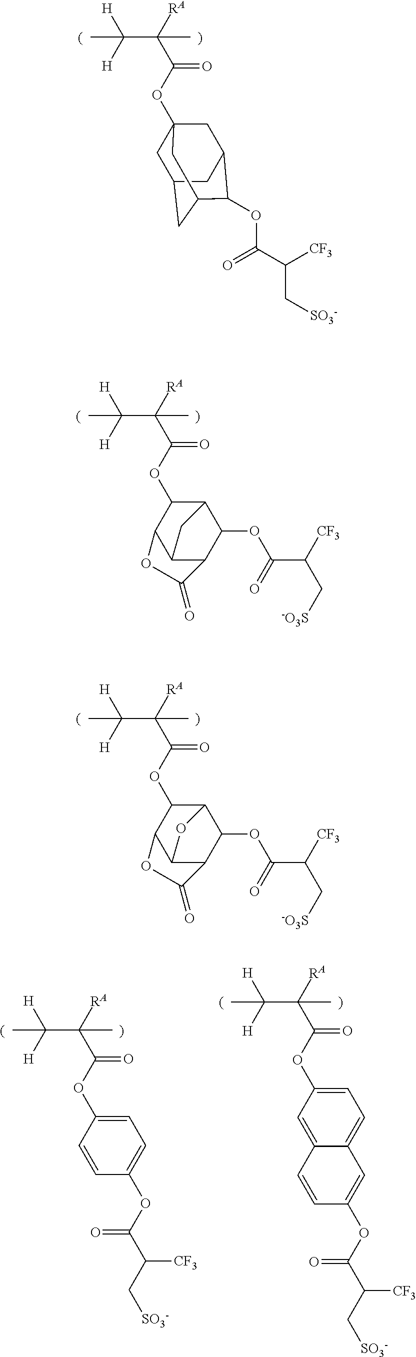

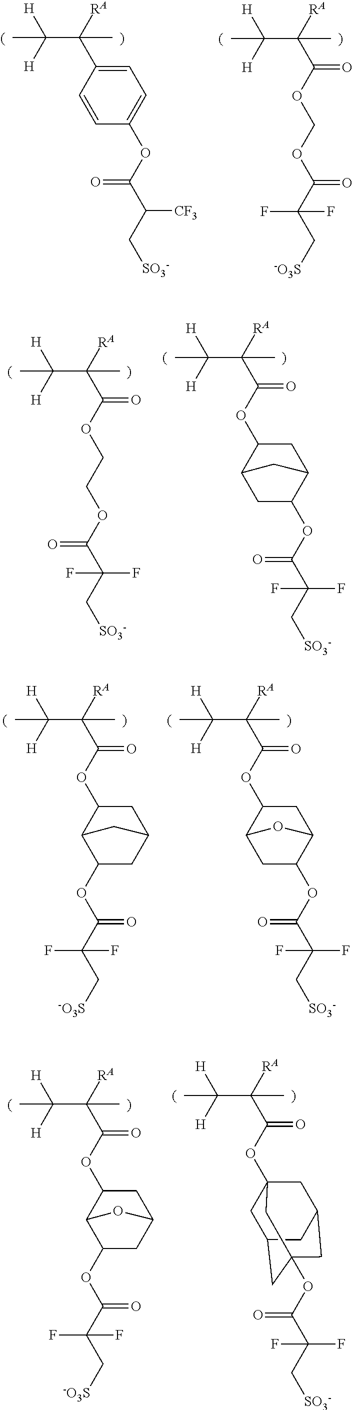

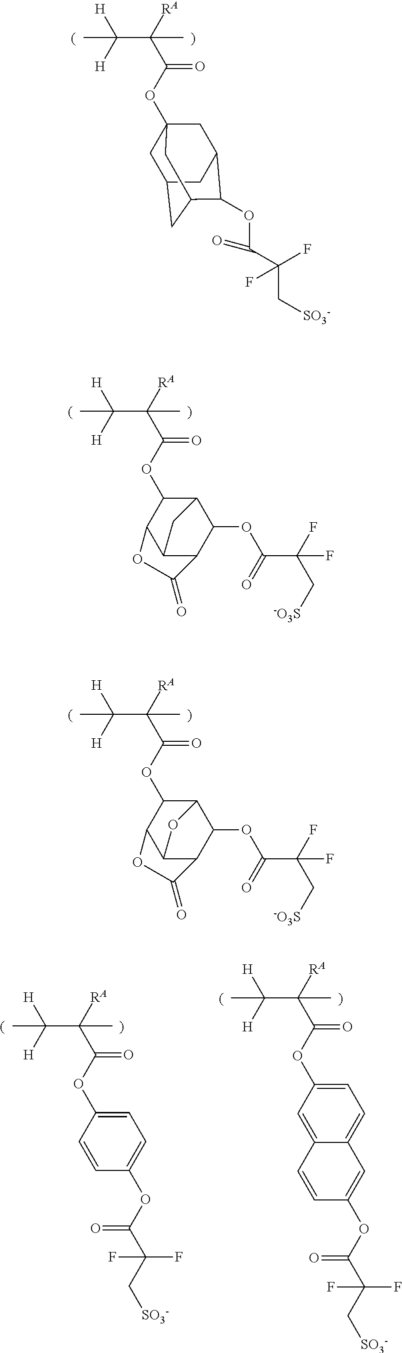

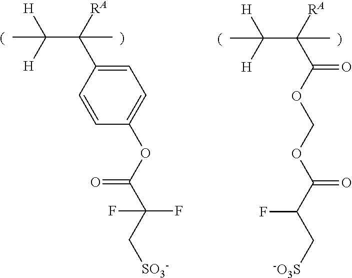

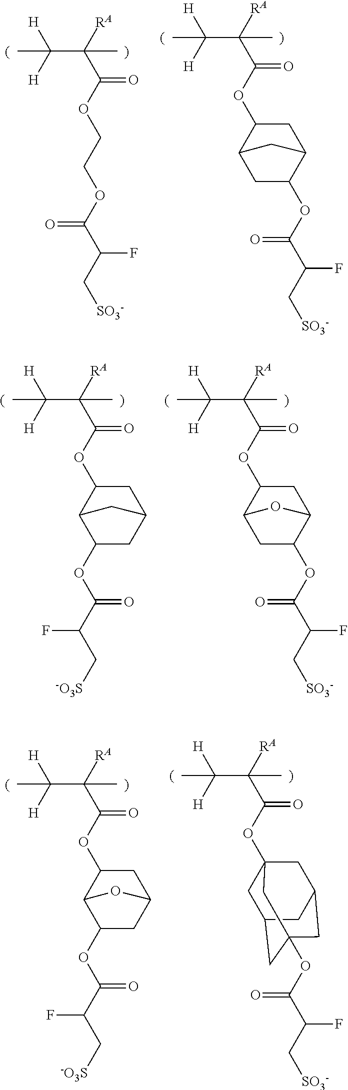

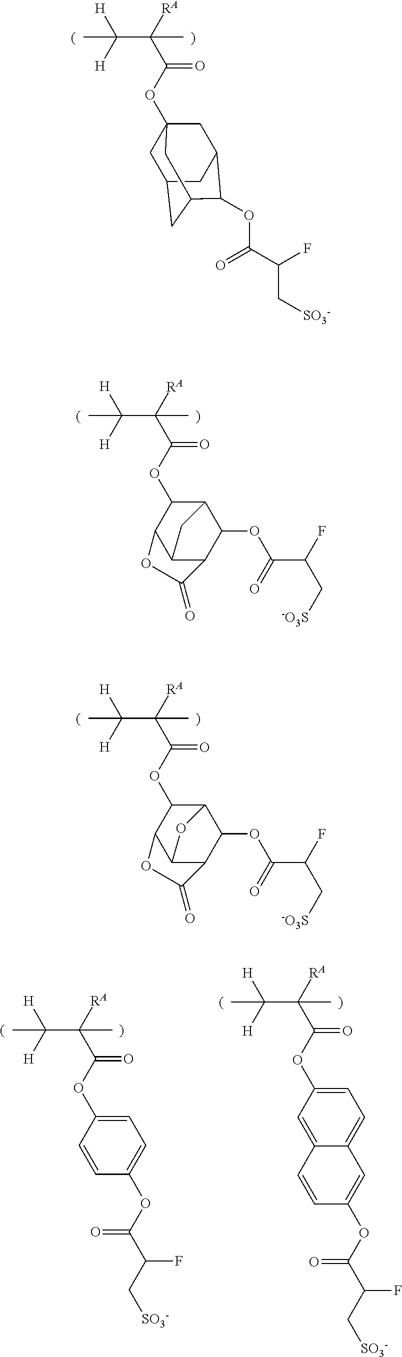

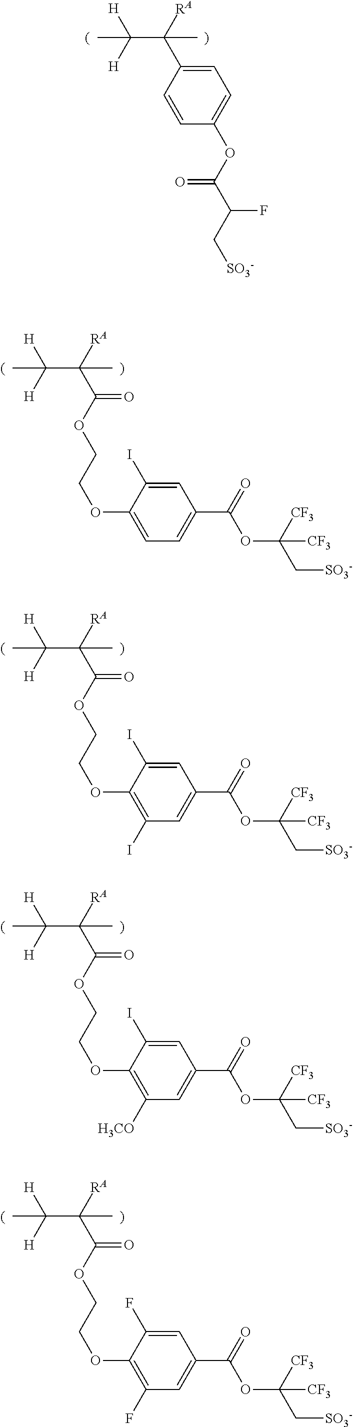

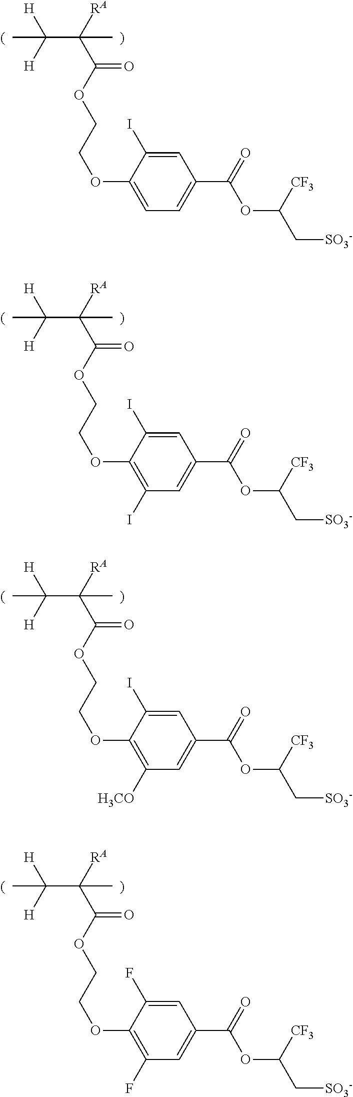

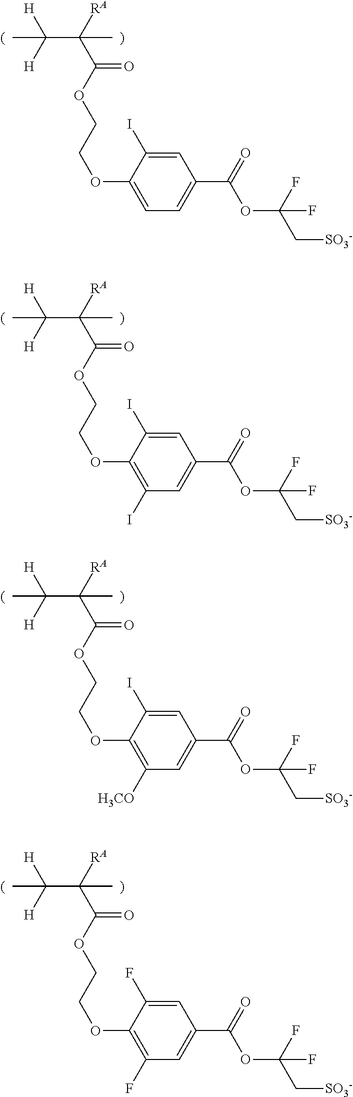

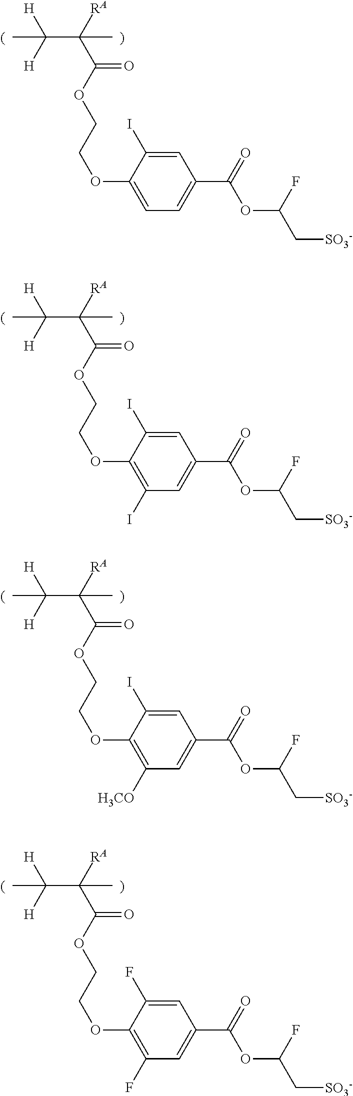

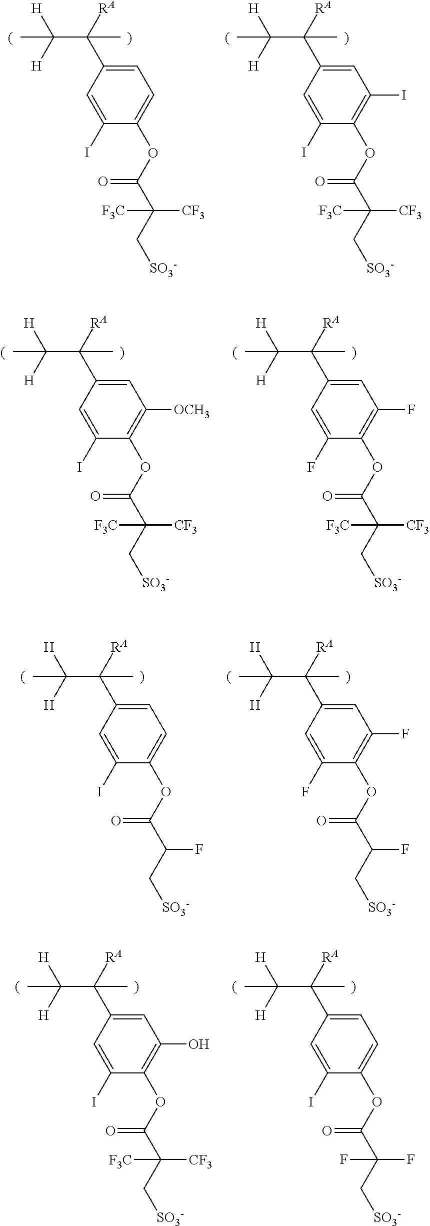

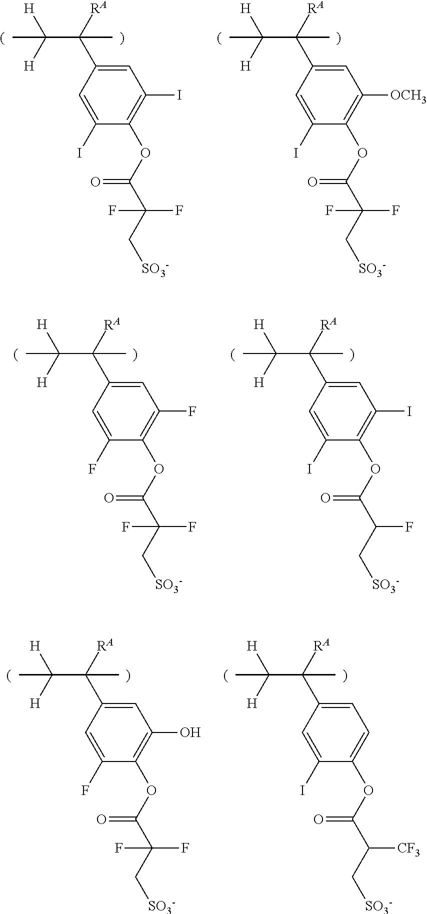

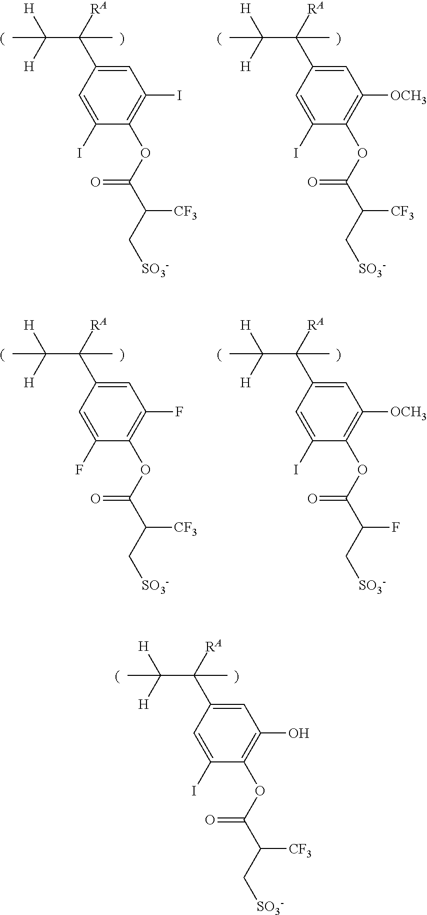

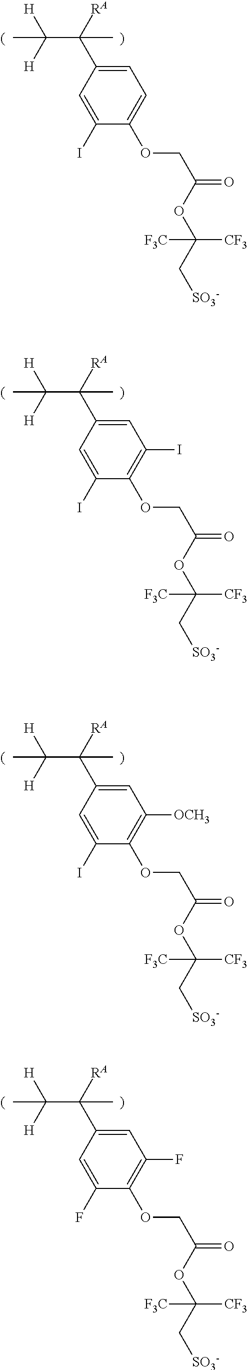

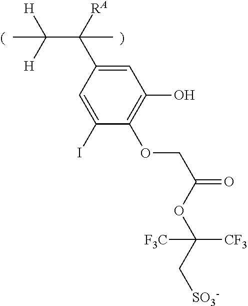

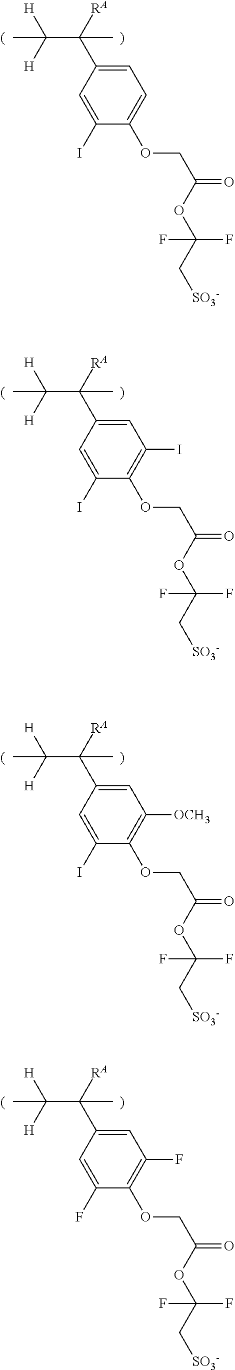

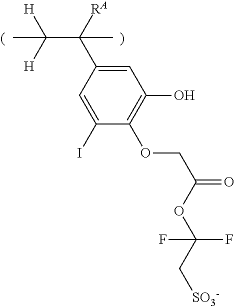

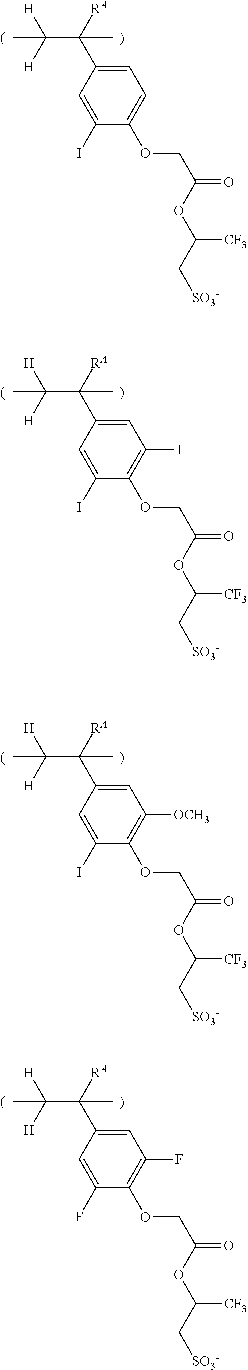

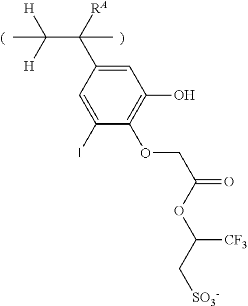

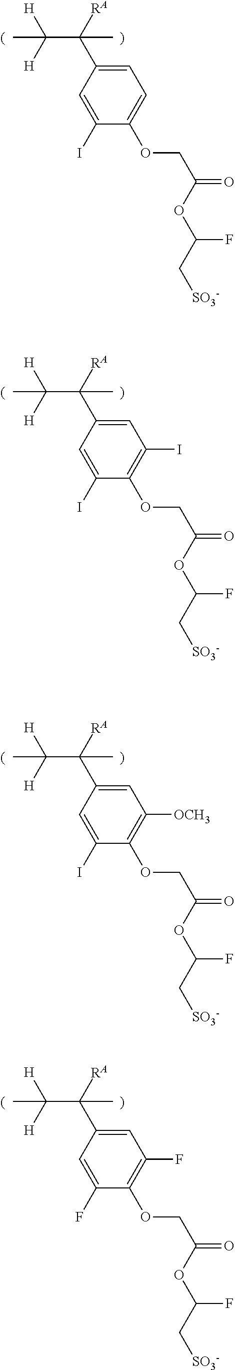

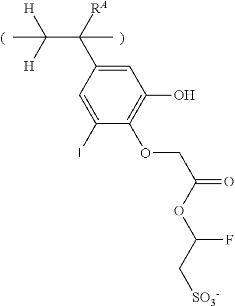

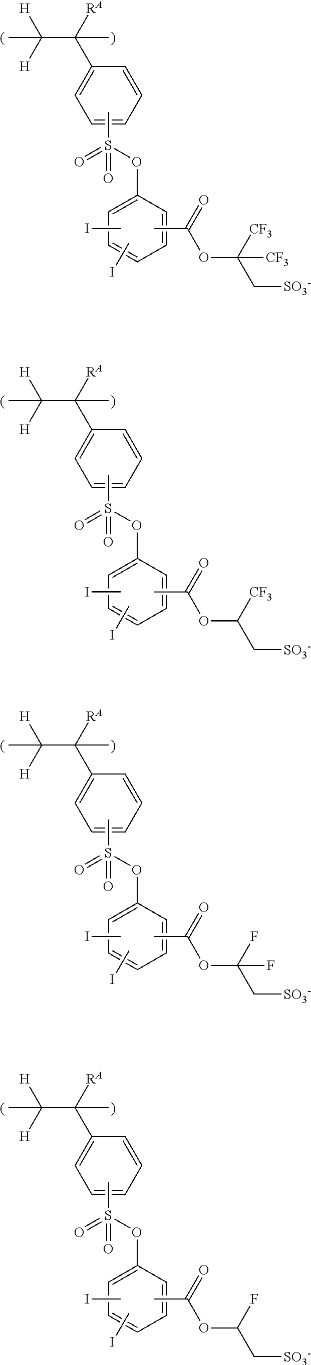

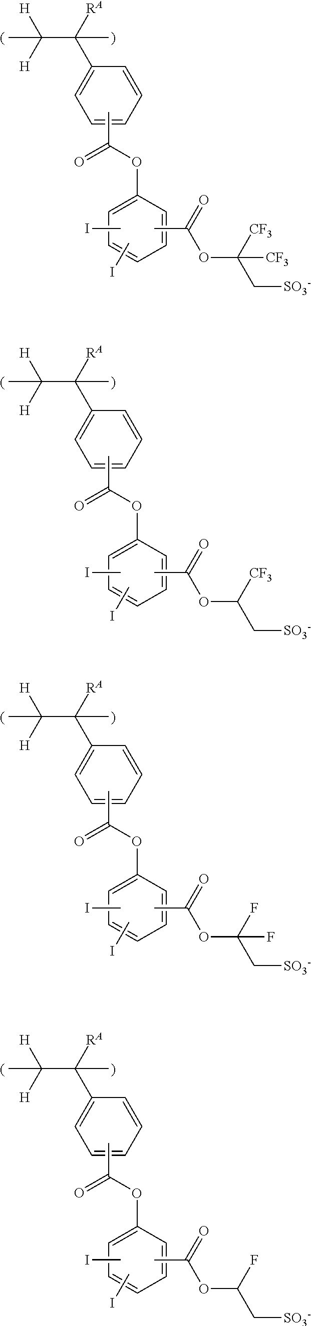

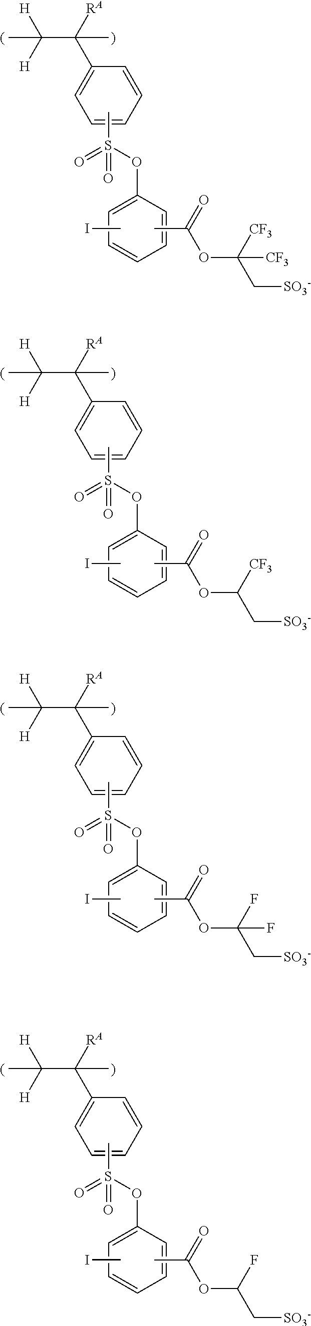

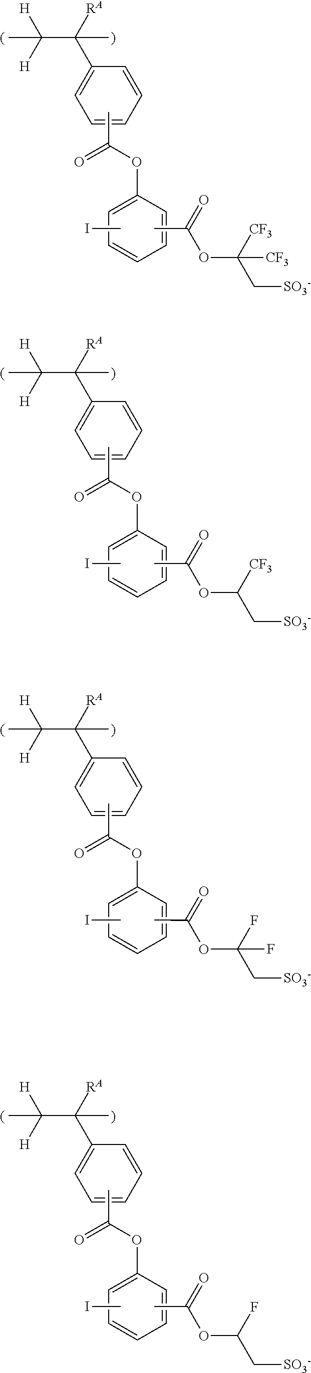

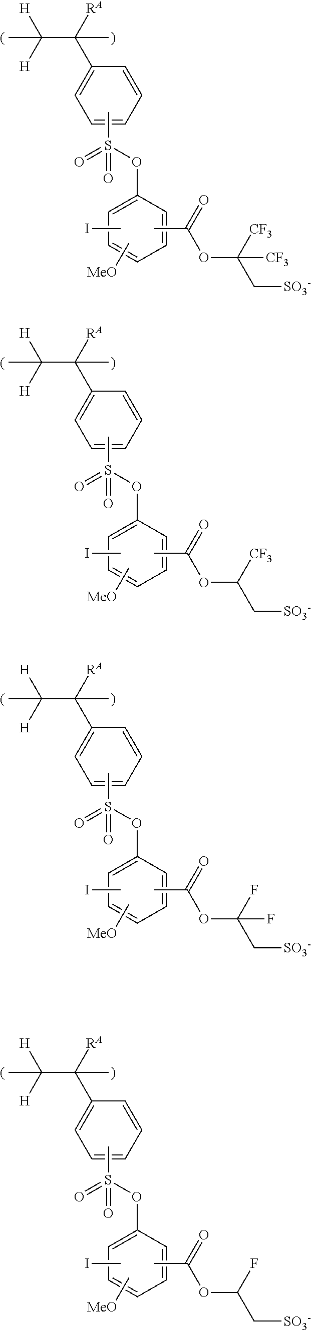

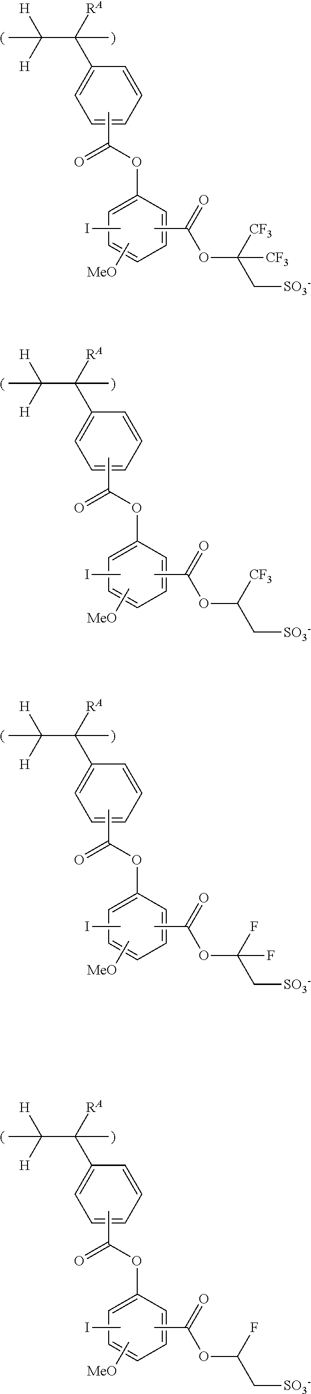

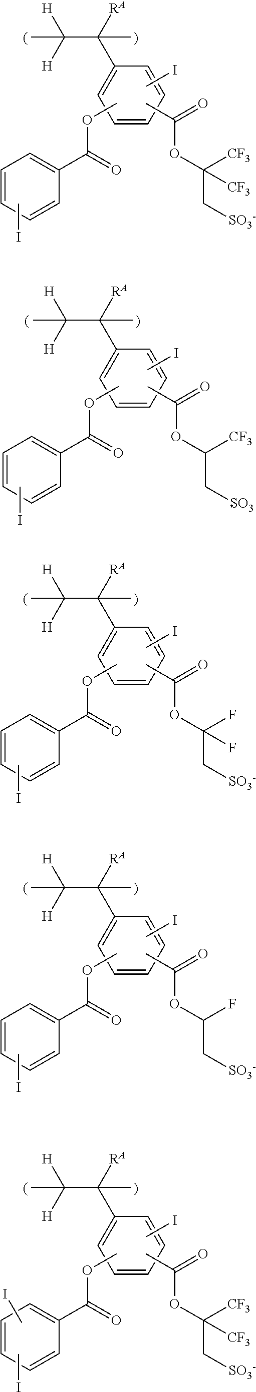

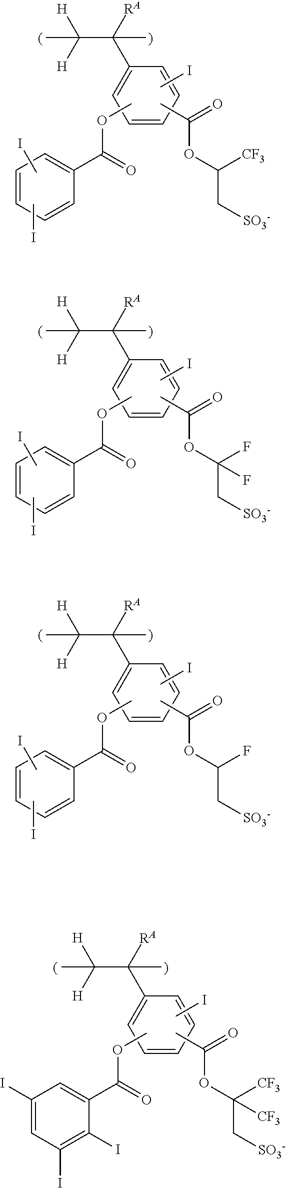

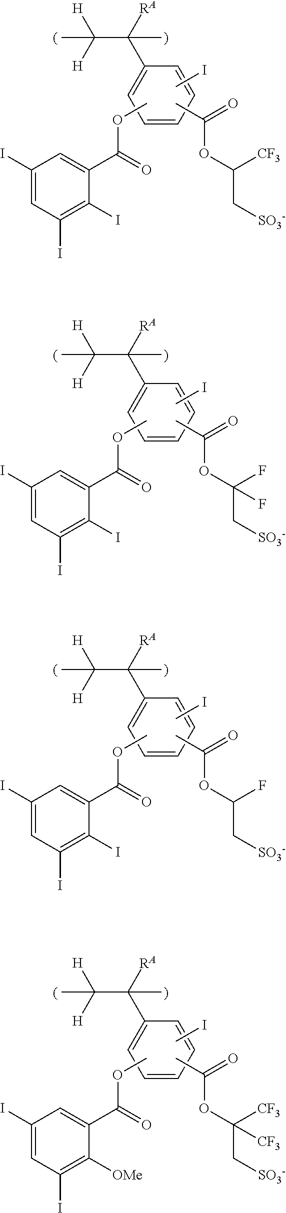

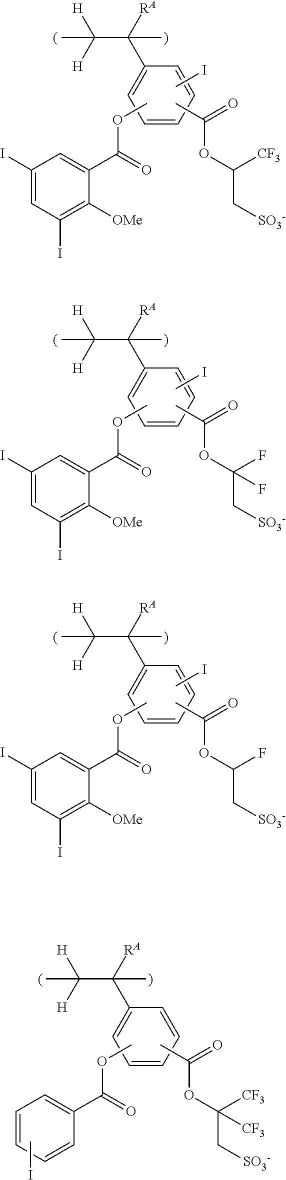

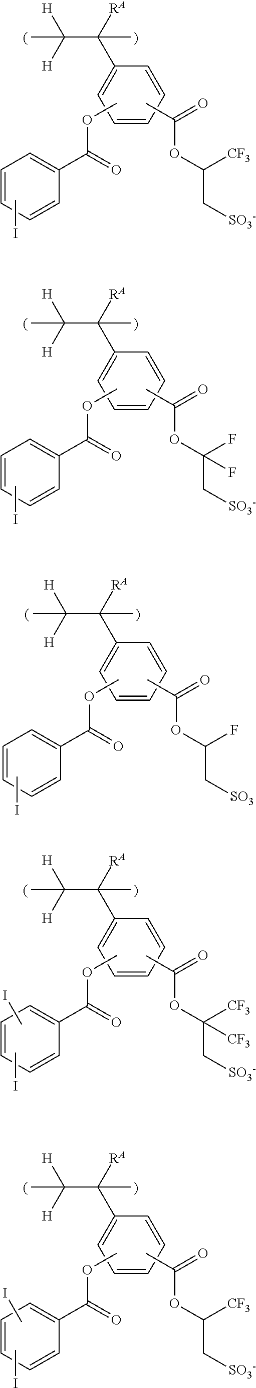

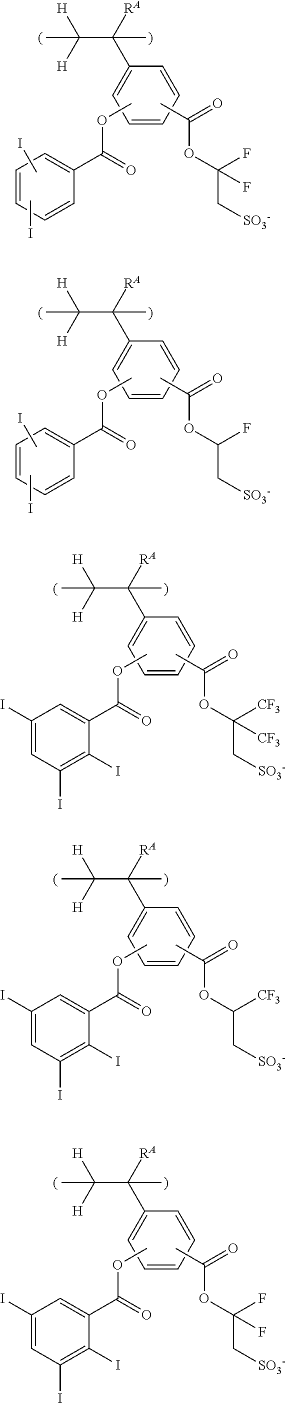

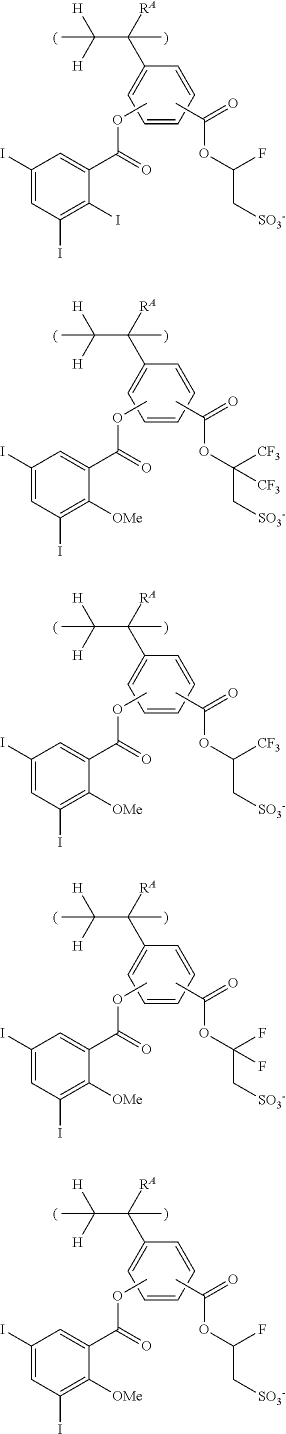

Specific examples of the anion in the repeating unit c4 are given below, but not limited thereto. In the formulae below, R^{A} is the same as above.

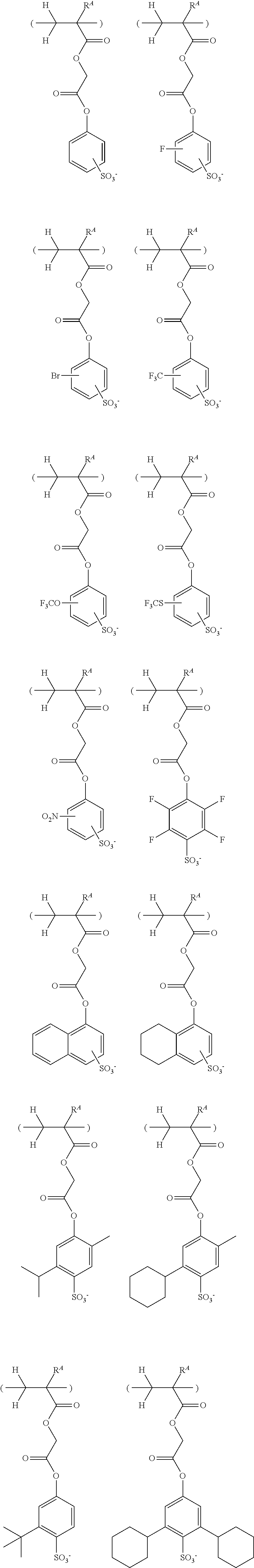

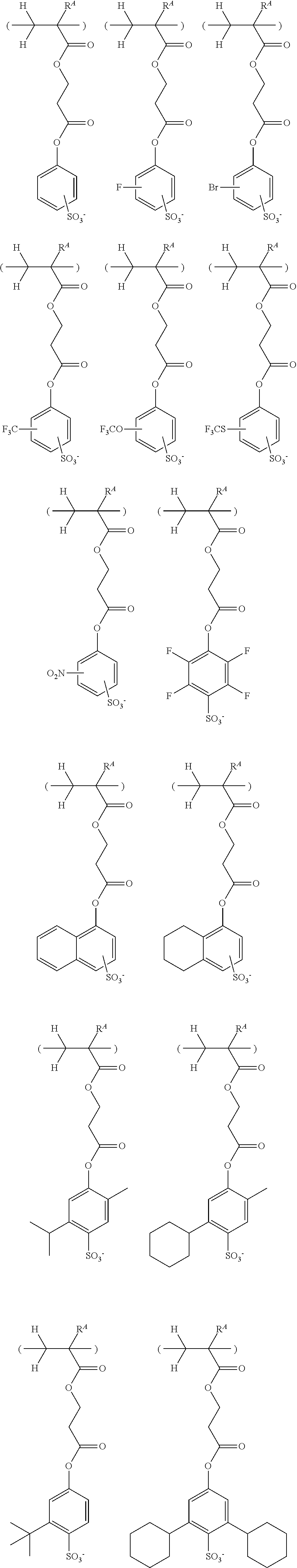

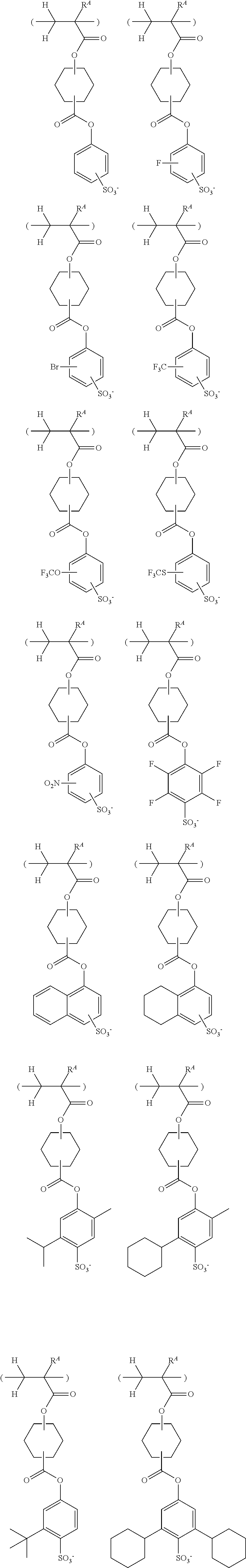

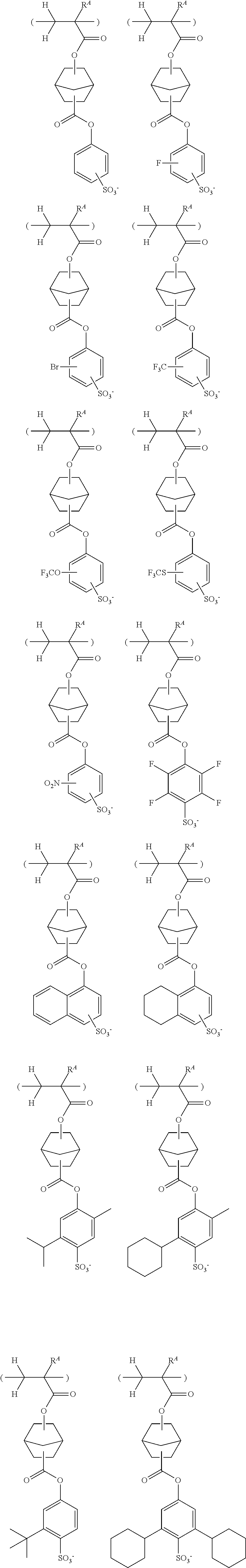

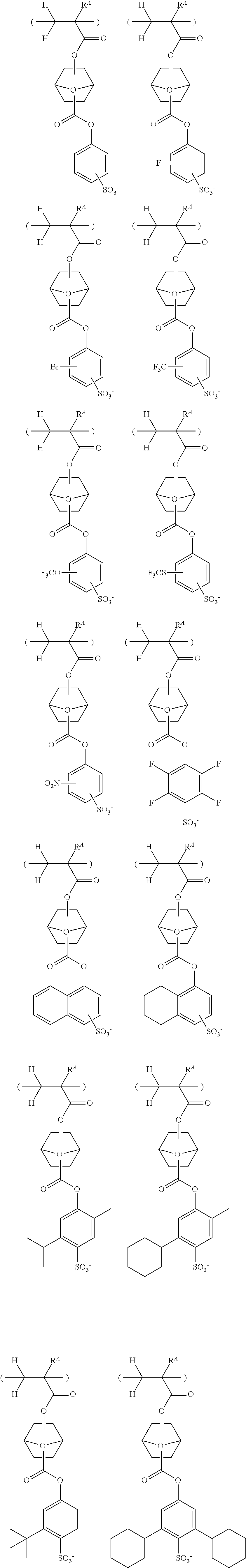

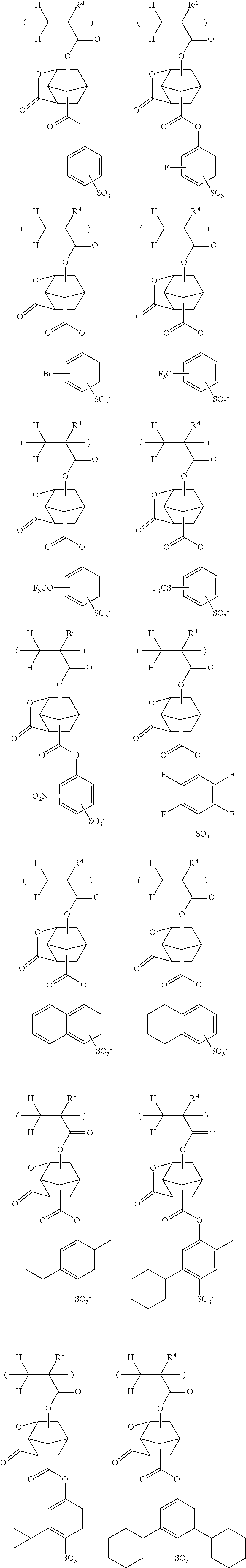

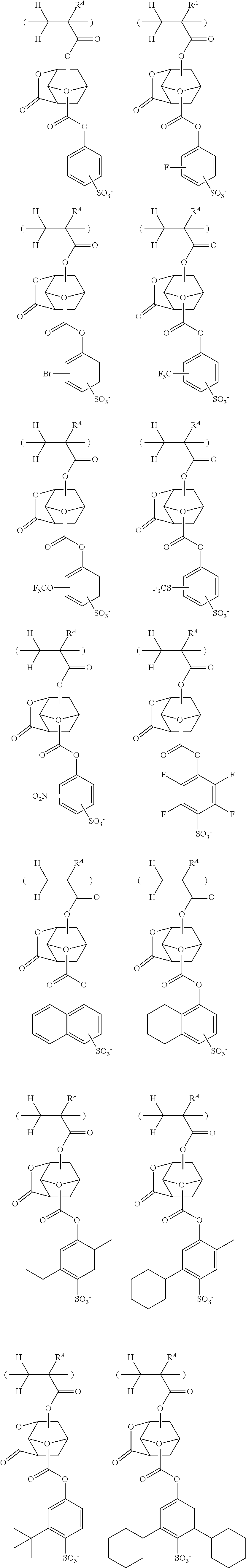

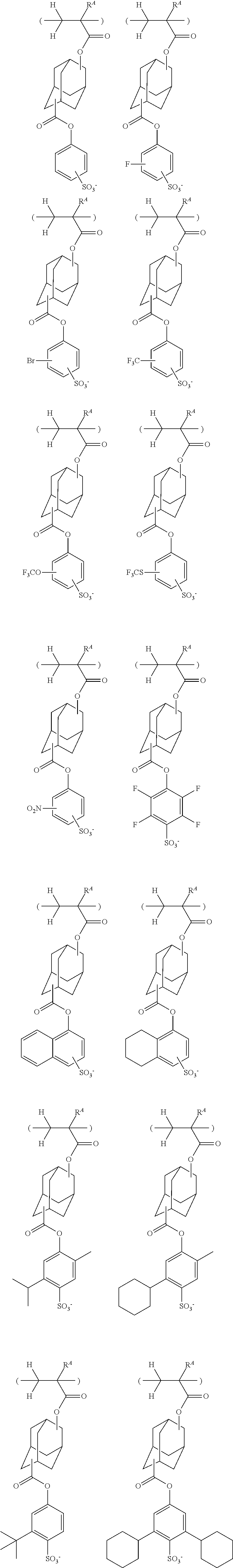

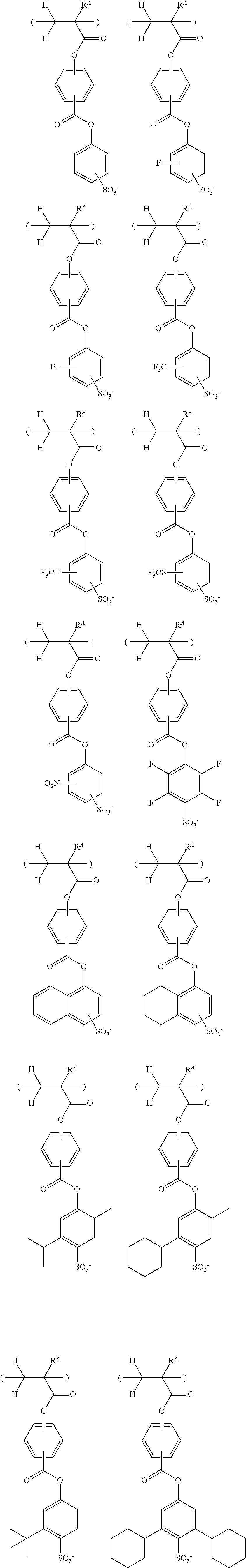

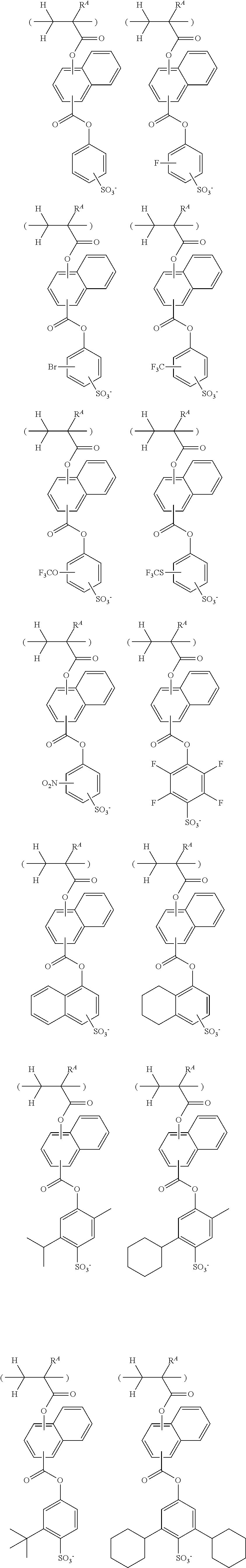

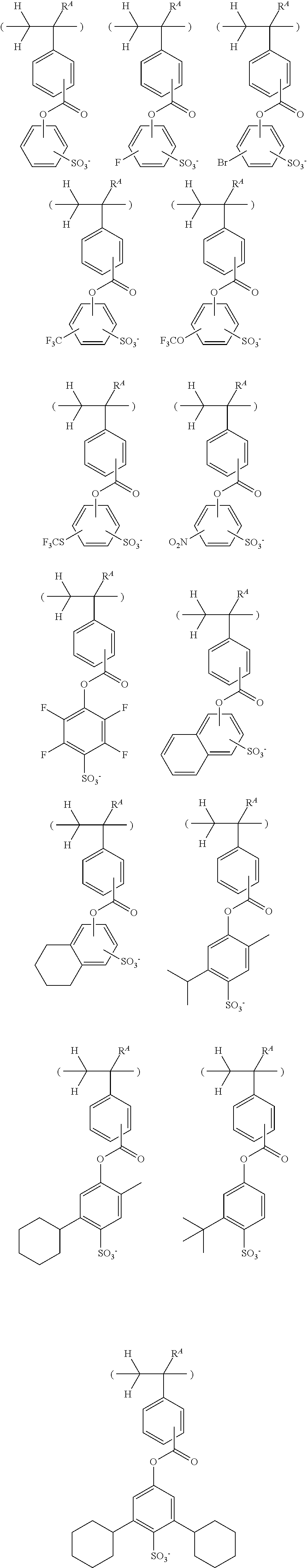

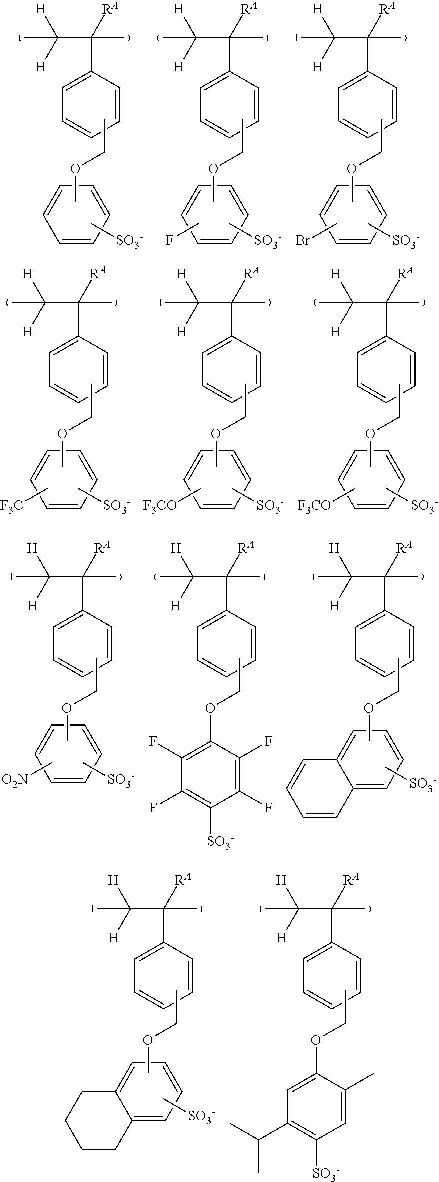

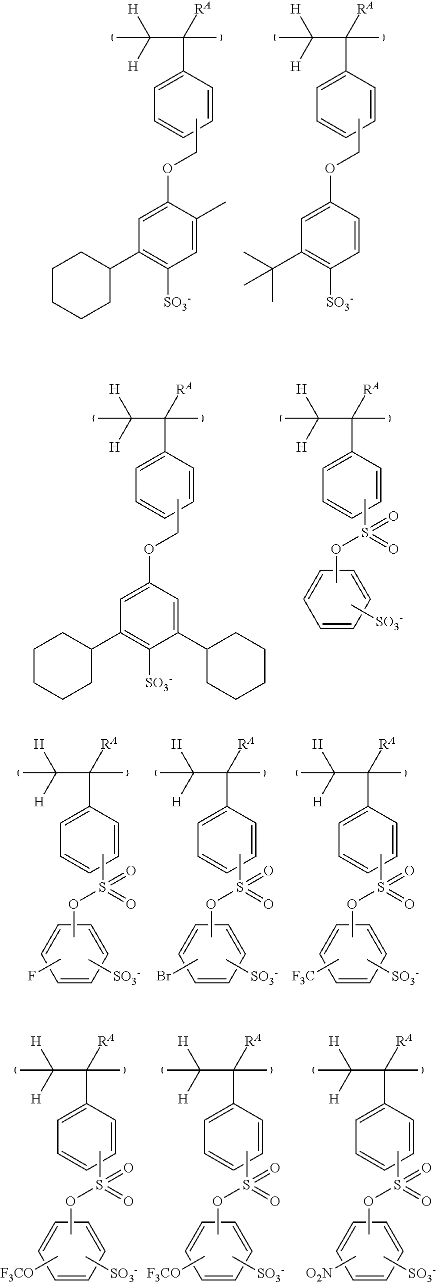

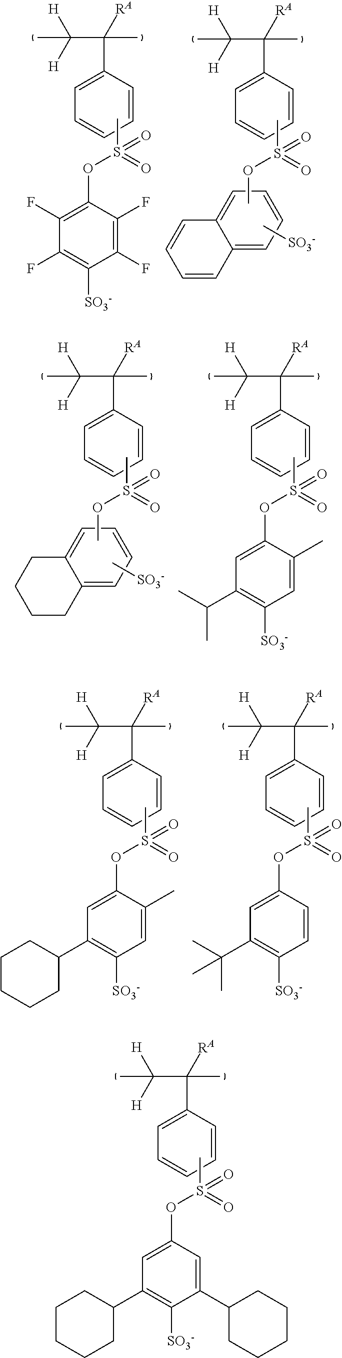

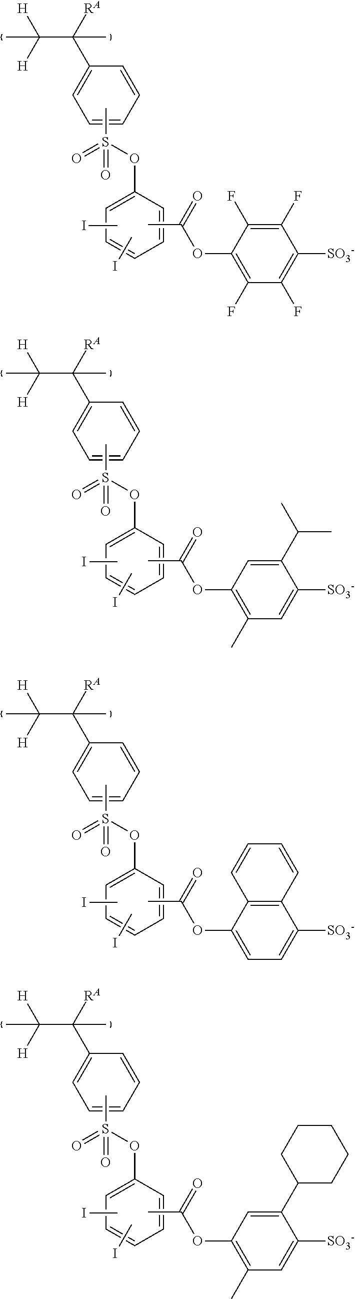

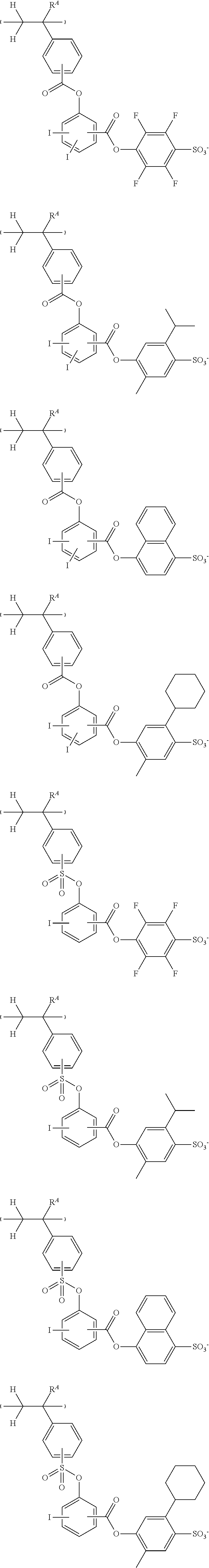

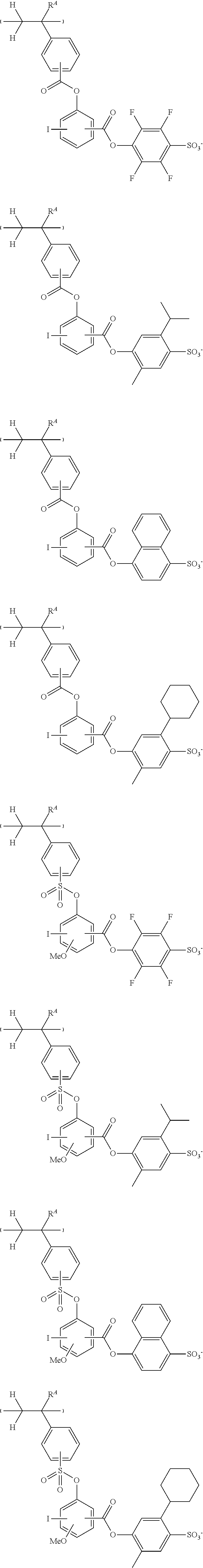

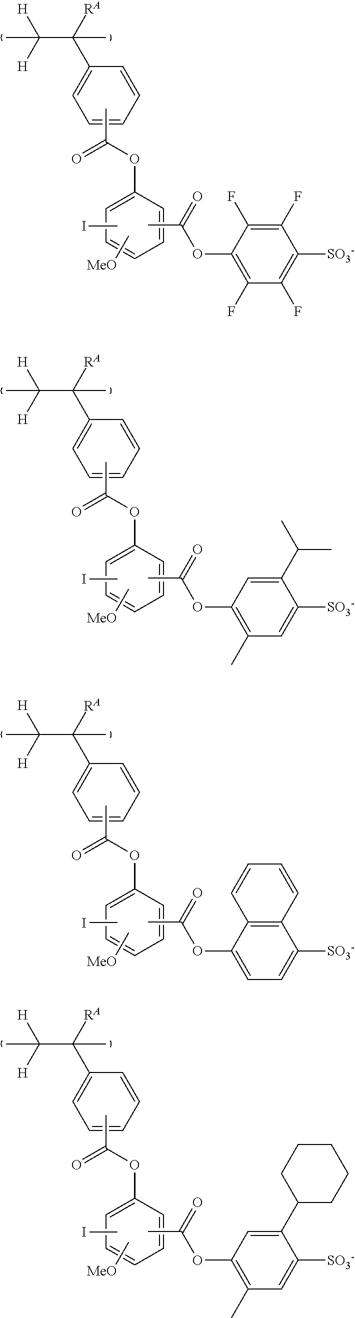

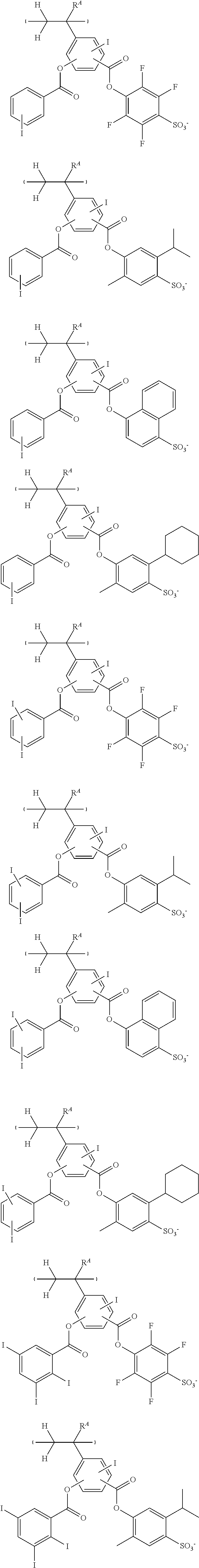

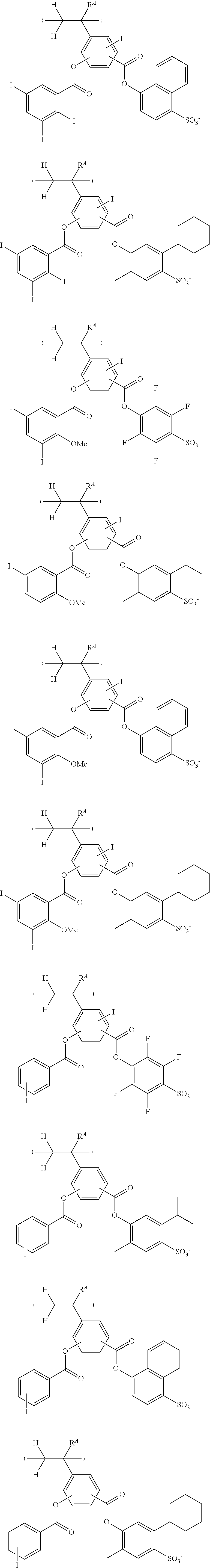

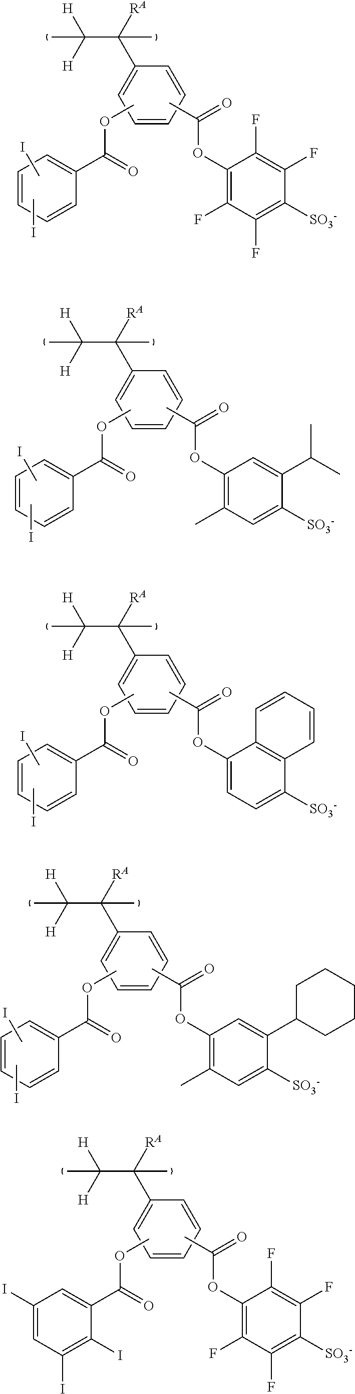

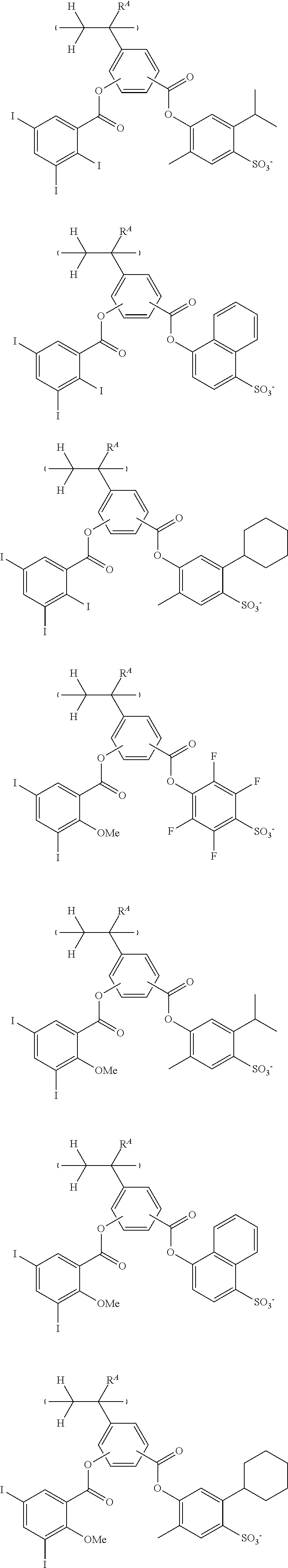

Specific examples of the anion in the repeating unit c5 are given below, but not limited thereto. In the formulae below, R^{A} is the same as above.

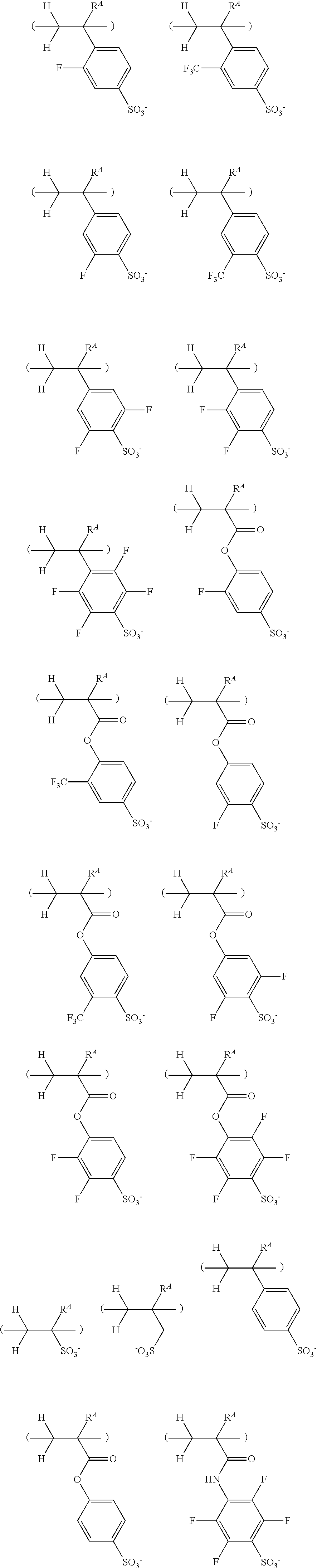

The sulfonium cation in A⁺ is preferably represented by formula (d3).

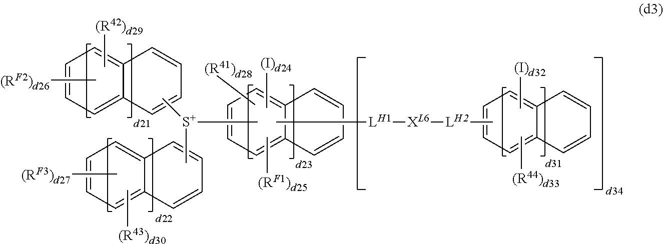

In formula (d3), “d21” is 0 or 1. The onium salt has a benzene ring when “d21” is 0, and a naphthalene ring when “d21” is 1. From the viewpoint of solvent solubility, a benzene ring with “d21” of 0 is preferable. “d22” is 0 or 1. The onium salt has a benzene ring when “d22” is 0, and a naphthalene ring when “d22” is 1. From the viewpoint of solvent solubility, a benzene ring with “d22” of 0 is preferable. “d23” is 0 or 1. The onium salt has a benzene ring when “d23” is 0, and a naphthalene ring when “d23” is 1. From the viewpoint of solvent solubility, a benzene ring with “d23” of 0 is preferable.

In formula (d3), “d24” is 0, 1, 2, 3, or 4. The larger the number of iodine atoms in the cation structure, the higher the absorption, especially for EUV, but the poorer the solvent solubility, raising a concern about precipitation in the resist composition. Thus, “d24” is preferably 0, 1, 2, or 3, and more preferably 0, 1, or 2.

In formula (d3), “d25” is 0, 1, 2, 3, or 4. From the viewpoint of raw material procurement, “d25” is preferably 0, 1, 2, or 3, and more preferably 0, 1, or 2. “d26” is 0, 1, 2, 3, 4, 5, or 6. From the viewpoint of raw material procurement, “d26” is preferably 0, 1, 2, or 3, and more preferably 0, 1, or 2. “d27” is 0, 1, 2, 3, 4, 5, or 6. From the viewpoint of raw material procurement, “d27” is preferably 0, 1, 2, or 3, and more preferably 0, 1, or 2.

In formula (d3), “d28” is 0, 1, or 2. From the viewpoint of raw material procurement, “d28” is preferably 0 or 1. “d29” is 0, 1, or 2. From the viewpoint of raw material procurement, “d29” is preferably 0 or 1. “d30” is 0, 1, or 2. From the viewpoint of raw material procurement, “d30” is preferably 0 or 1.

In formula (d3), “d31” is 0 or 1. The onium salt has a benzene ring when “d31” is 0, and a naphthalene ring when “d31” is 1. From the viewpoint of solvent solubility, a benzene ring with “d31” of 0 is preferable.

In formula (d3), “d32” is 0, 1, 2, 3, or 4. The larger the number of iodine atoms in the cation structure, the higher the absorption, especially for EUV, but the poorer the solvent solubility, raising a concern about precipitation in the resist composition. Thus, “d32” is preferably 0, 1, 2, or 3, and more preferably 0, 1, or 2.

In formula (d3), “d33” is 0, 1, or 2. From the viewpoint of raw material procurement, “d33” is preferably 0 or 1. “d34” is 0, 1, or 2. From the viewpoint of synthesis, “d34” is preferably 0 or 1.

However, when “d21” is 0, 0≤d26+d29≤4, and when “d21” is 1, 0≤d26+d29≤6. When “d22” is 0, 0≤d27+d30≤4, and when “d22” is 1, 0≤d27+d30≤6. When “d23” is 0, 1≤d24+d25+d28+d34≤4, and when “d23” is 1, 1≤d24+d25+d28+d4≤6. When “d31” is 0, 0≤d32+d33≤4, and when “d31” is 1, 0≤d32+d33 ≤6. In addition, d24+d32≥1.

In formula (d3), R^{F1} to R^{F3} are each independently a fluorine atom, a fluorinated saturated hydrocarbyl group having 1 to 6 carbon atoms, a fluorinated saturated hydrocarbyloxy group having 1 to 6 carbon atoms, or a fluorinated saturated hydrocarbylthio group having 1 to 6 carbon atoms. Of these, a trifluoromethyl group, a trifluoromethoxy group, and a trifluorothiomethoxy group are preferable. When “d25” is 2 or more, R^{F1} are the same as or different from each other. When “d26” is 2 or more, RF² are the same as or different from each other. When “d27” is 2 or more, R^{F3} are the same as or different from each other.

In formula (d3), R⁴¹ to R⁴⁴ are each a halogen atom other than an iodine atom and a fluorine atom, a nitro group, a cyano group, a hydrocarbyl group having 1 to 20 carbon atoms and optionally comprising a heteroatom, a hydrocarbyloxy group having 1 to 20 carbon atoms and optionally comprising a heteroatom, or a hydrocarbylthio group having 1 to 20 carbon atoms and optionally comprising a heteroatom. The hydrocarbyl group and hydrocarbyl moieties of the hydrocarbyloxy group and hydrocarbylthio group may be either saturated or unsaturated and linear, branched, or cyclic. Specific examples thereof include the same hydrocarbyl group represented by R¹ in the description of formula (1) as illustrated. Some or all of the hydrogen atoms in the hydrocarbyl group and hydrocarbyl moieties of the hydrocarbyloxy group and hydrocarbylthio group are optionally substituted with a group containing a heteroatom such as an oxygen atom, a sulfur atom, a nitrogen atom, or a halogen atom, and some of —CH₂— in the hydrocarbyl group are optionally substituted with a group containing a heteroatom such as an oxygen atom, a sulfur atom, or a nitrogen atom. As a result, the hydrocarbyl group may include, for instance, a hydroxy group, a cyano group, a fluorine atom, a chlorine atom, a bromine atom, an iodine atom, a carbonyl group, an ether bond, an ester bond, a sulfonate ester bond, a carbonate bond, a lactone ring, a sultone ring, a carboxylic anhydride(—C(═O)—O—C(═O)—), and a haloalkyl group.

When “d28” is 2, two R⁴¹ are the same as or different from each other, and two R⁴¹ are optionally bonded to each other to form a ring together with carbon atoms to which the R⁴¹ are bonded. When “d29” is 2, two R⁴² are the same as or different from each other, and two R⁴² are optionally bonded to each other to form a ring together with carbon atoms to which the R⁴² are bonded. When “d30” is 2, two R⁴³ are the same as or different from each other, and two R⁴³ are optionally bonded to each other to form a ring together with carbon atoms to which the R⁴³ are bonded. When “d33” is 2, two R⁴⁴ are the same as or different from each other, and two R⁴⁴ are optionally bonded to each other to form a ring together with carbon atoms to which the R⁴⁴ are bonded. Specific examples of the ring formed at this time include a cyclopropane ring, a cyclobutane ring, a cyclopentane ring, a cyclohexane ring, a norbornane ring, and an adamantane ring. Some or all of the hydrogen atoms in the ring are optionally substituted with a group containing a heteroatom such as an oxygen atom, a sulfur atom, a nitrogen atom, or a halogen atom, and some of —CH₂— in the ring are optionally substituted with a group containing a heteroatom such as an oxygen atom, a sulfur atom, or a nitrogen atom. As a result, the ring may include, for instance, a hydroxy group, a fluorine atom, a chlorine atom, a bromine atom, an iodine atom, a cyano group, a carbonyl group, an ether bond, an ester bond, a sulfonate ester bond, a carbonate bond, a lactone ring, a sultone ring, a carboxylic anhydride(—C(═O)—O—C(═O)—), and a haloalkyl group.

In addition, aromatic rings directly bonded to S⁺ in the sulfonium cation represented by formula (d3) are optionally bonded to each other to form a ring together with S⁺. At this point, specific examples of the structure of the ring include those represented by formulae below:

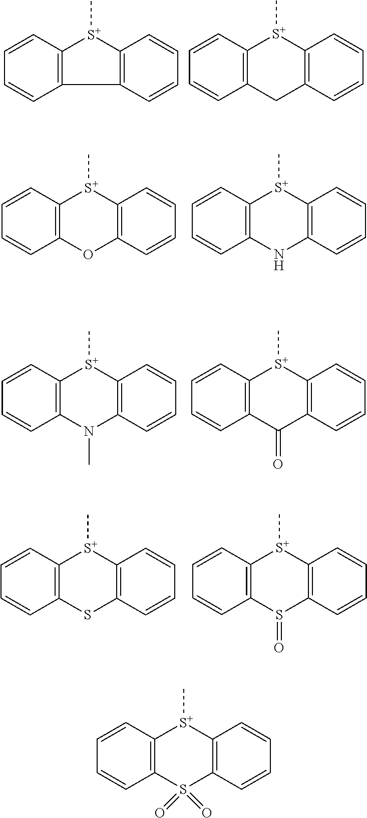

wherein the dashed line represents a bond.

In formula (d3), L^{H1} and L^{H2} are each independently a single bond, an ether bond, an ester bond, an amide bond, a sulfonate ester bond, a sulfonate amide bond, a carbonate bond, or a carbamate bond. Of these, L^{H1} is preferably a single bond, an ether bond, an ester bond, or a sulfonate ester bond, and more preferably an ester bond or a sulfonate ester bond. L^{H2} is preferably a single bond, an ether bond, or an ester bond, and more preferably a single bond.

In formula (d3), X^{L6} is a single bond or a hydrocarbylene group having 1 to 40 carbon atoms and optionally comprising a heteroatom. The hydrocarbylene group may be linear, branched, or cyclic. Specific examples thereof include an alkandiyl group, a cyclic saturated hydrocarbylene group, and an arylene group. Specific examples of the heteroatom include an oxygen atom, a nitrogen atom, and a sulfur atom. Specific examples of the hydrocarbylene group having 1 to 40 carbon atoms and optionally comprising a heteroatom, represented by X^{L6} include X^{L}-0 to X^{L}-58, which are illustrated as specific examples of the hydrocarbylene groups having 1 to 40 carbon atoms and optionally comprising a heteroatom, represented by X^{L1} and X^{L2}, in the description of formula (Z). Of these, X^{L}-0 to X^{L}-22, X^{L}-29 to X^{L}-34, and X^{L}-47 to X^{L}-58 are preferable.

The sulfonium cation represented by formula (d3) is preferably represented by formula (d3-1):

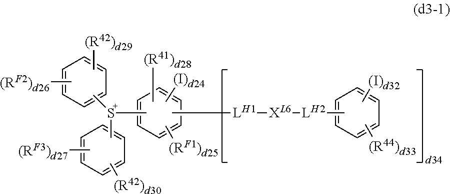

wherein “d24” to “d30”, “d32” to “d34”, R^{F1} to R^{F3}, R⁴¹ to R⁴⁴, L^{H1}, L^{H2}, and X^{L6} are the same as above.

The sulfonium cation represented by formula (d3-1) is preferably represented by formula (d3-2):

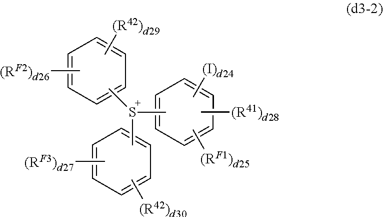

wherein “d24” to “d30”, R^{F1} to R^{F3}, and R^{ct6} to R^{ct3} are the same as above.

Specific examples of the sulfonium cation represented by formula (3) are given below, but not limited thereto. In formulae below, Me represents a methyl group.

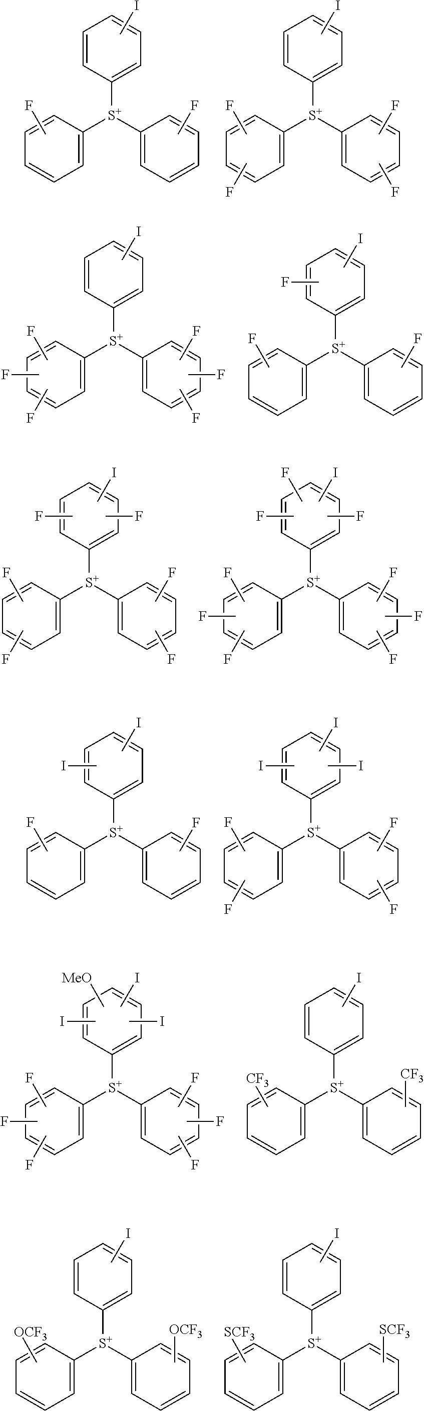

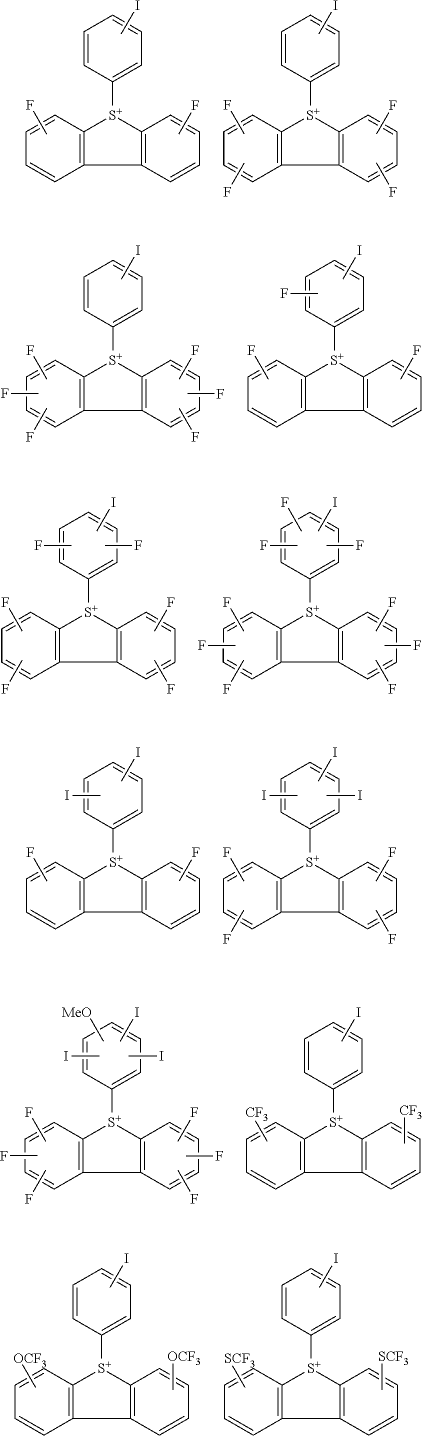

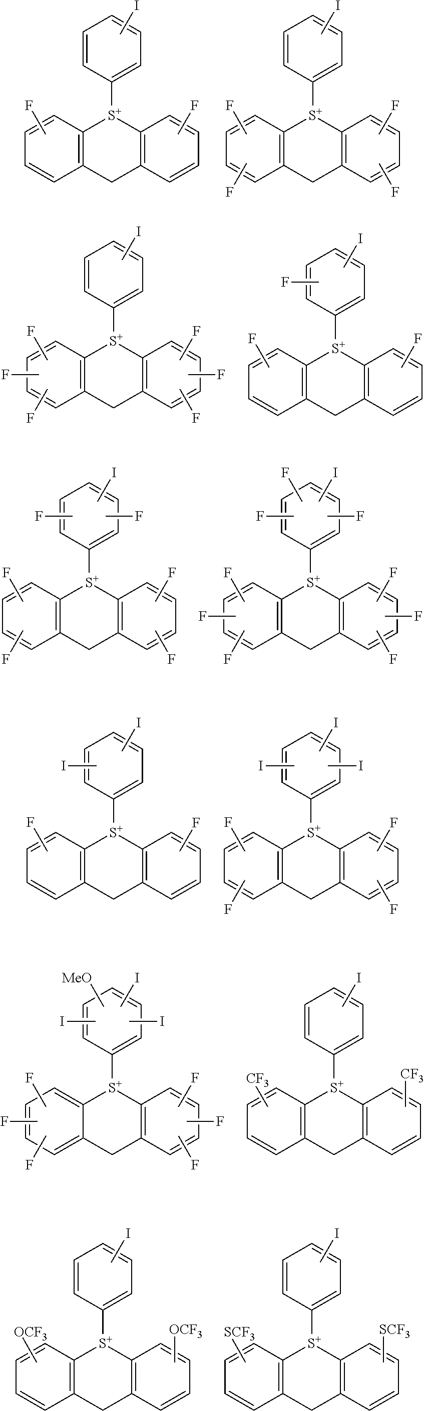

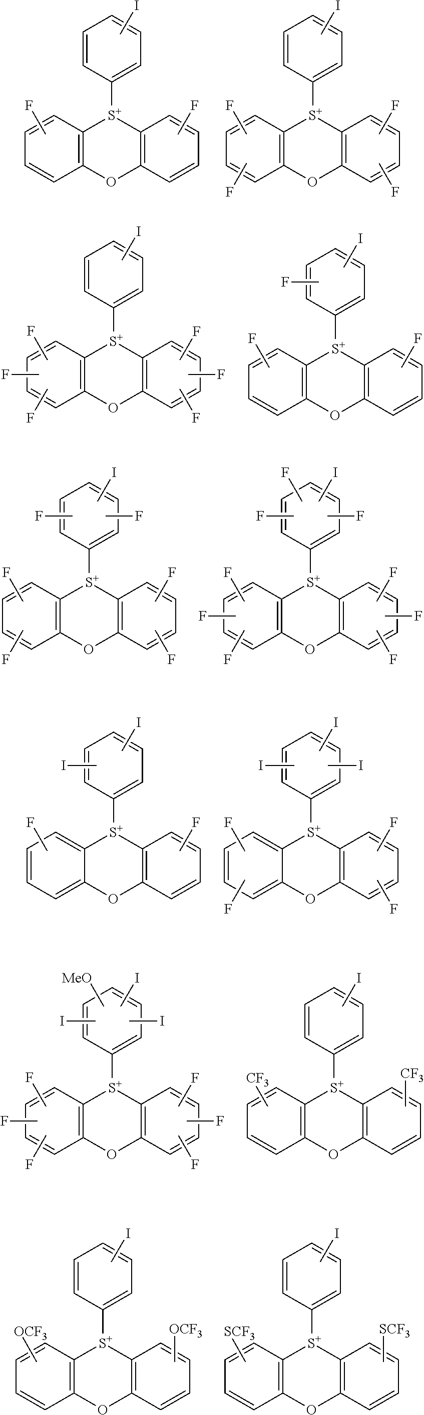

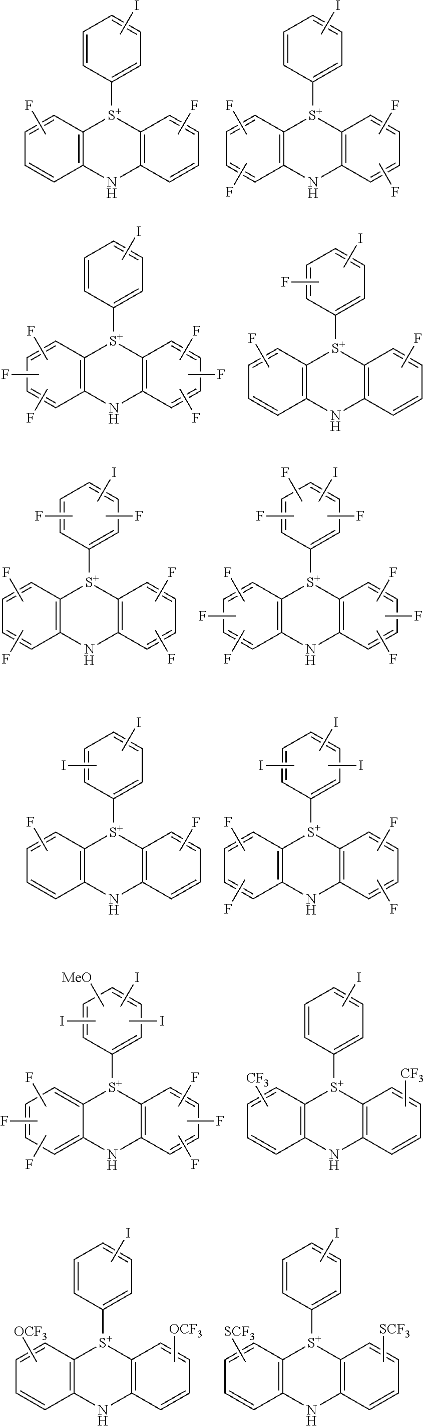

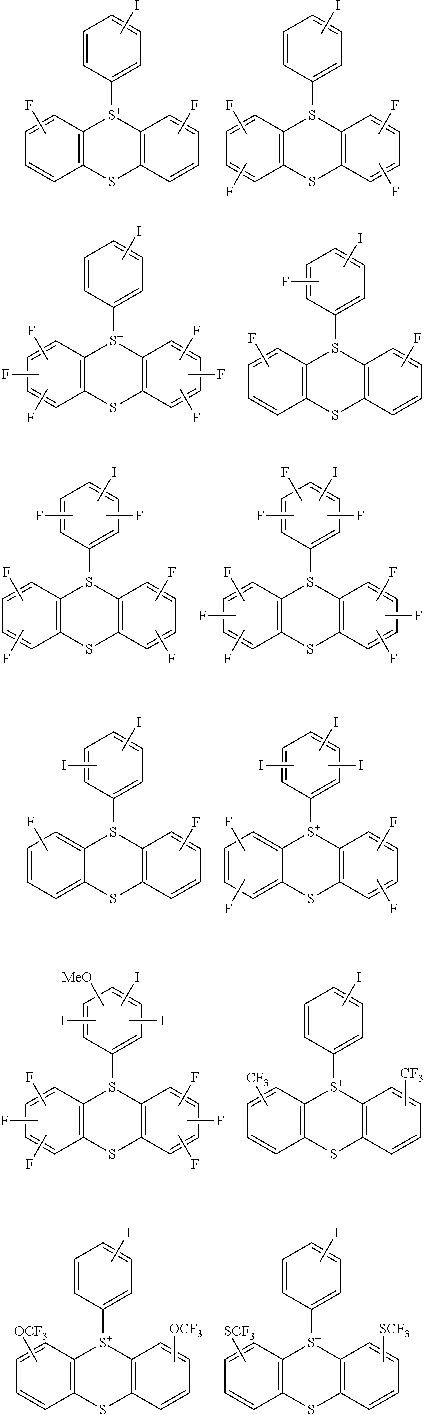

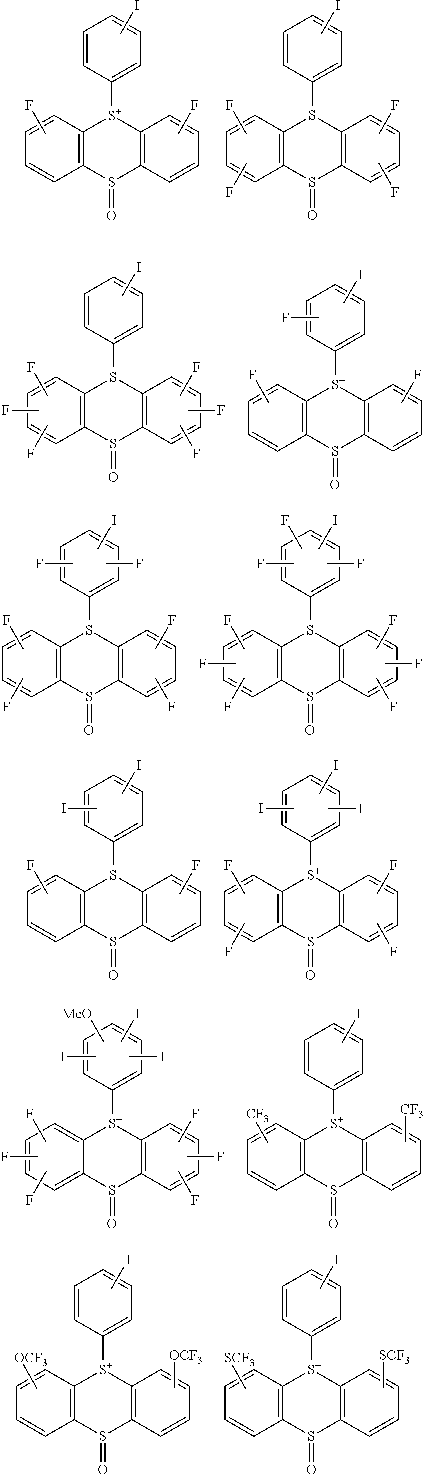

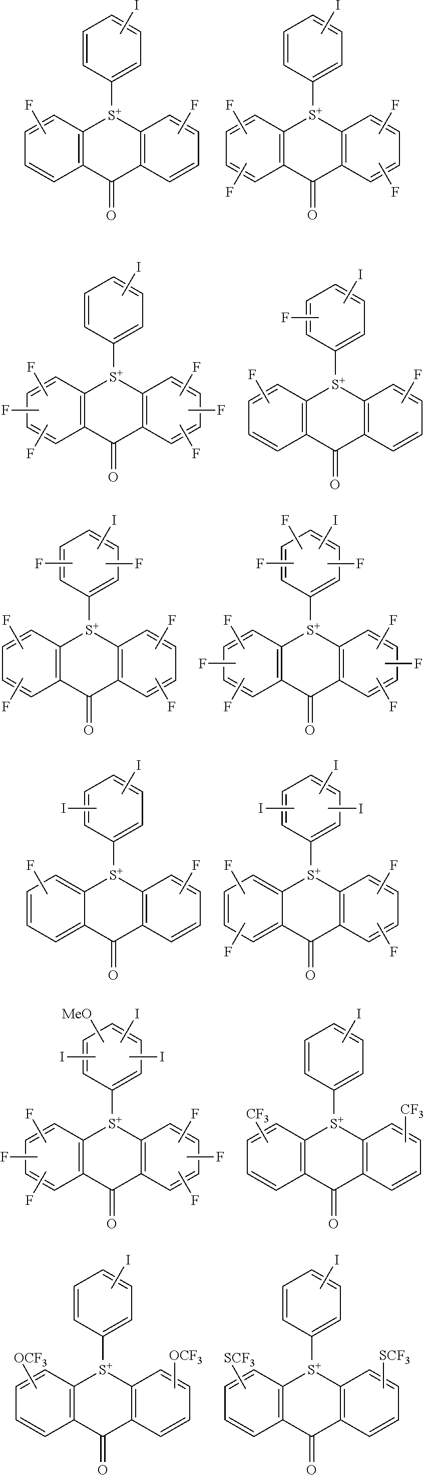

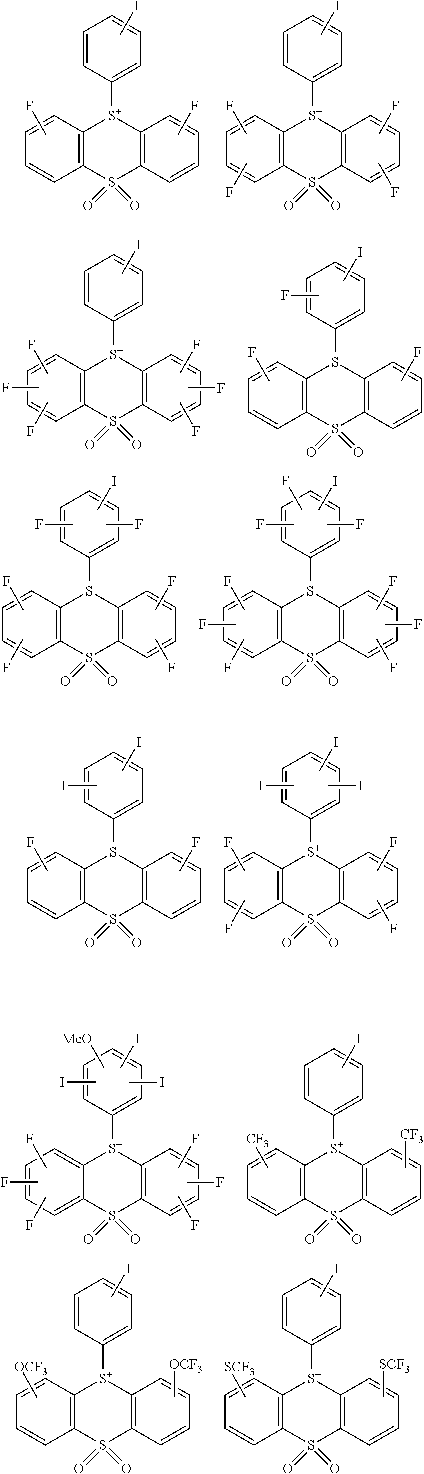

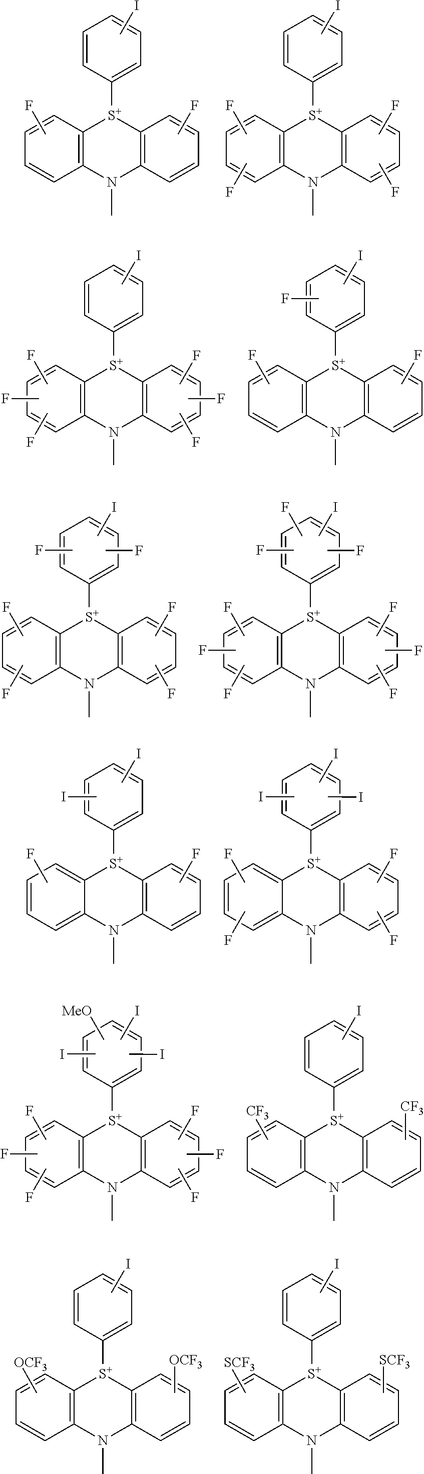

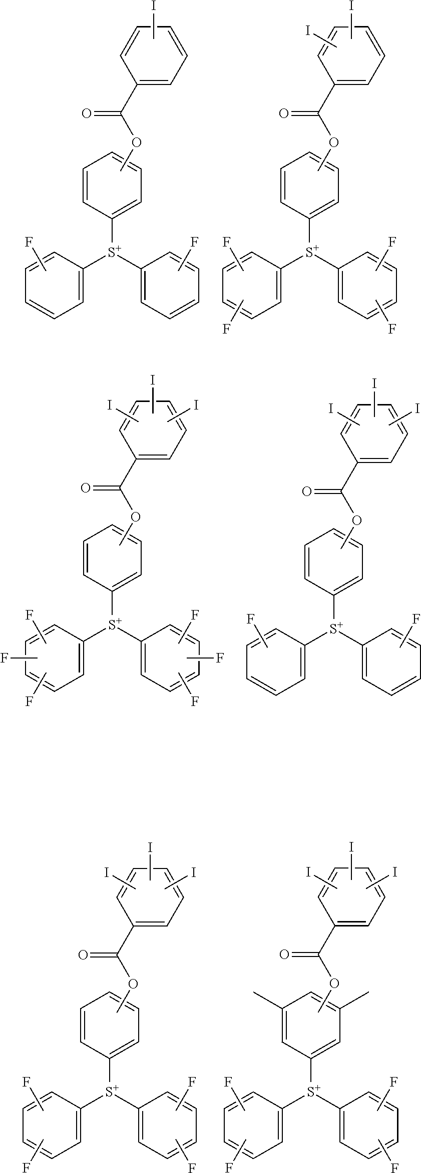

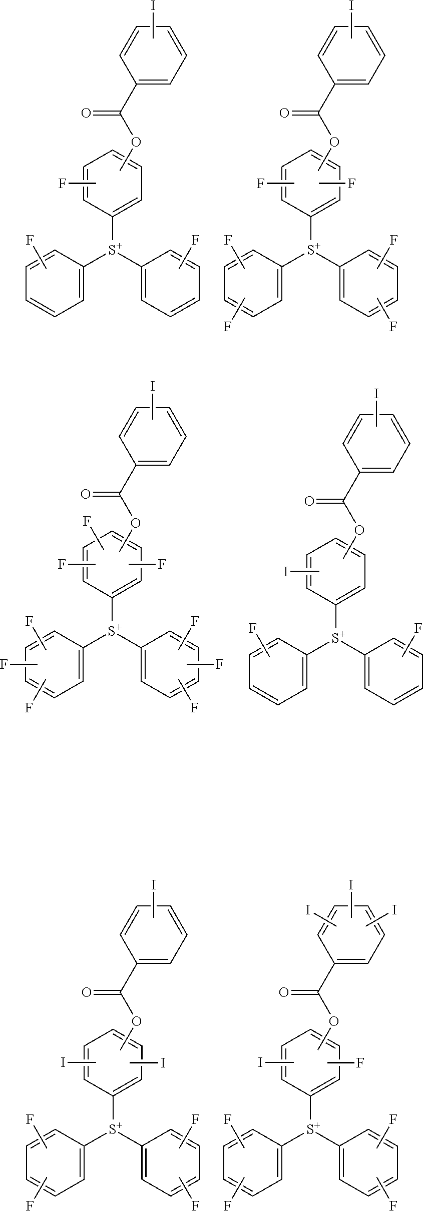

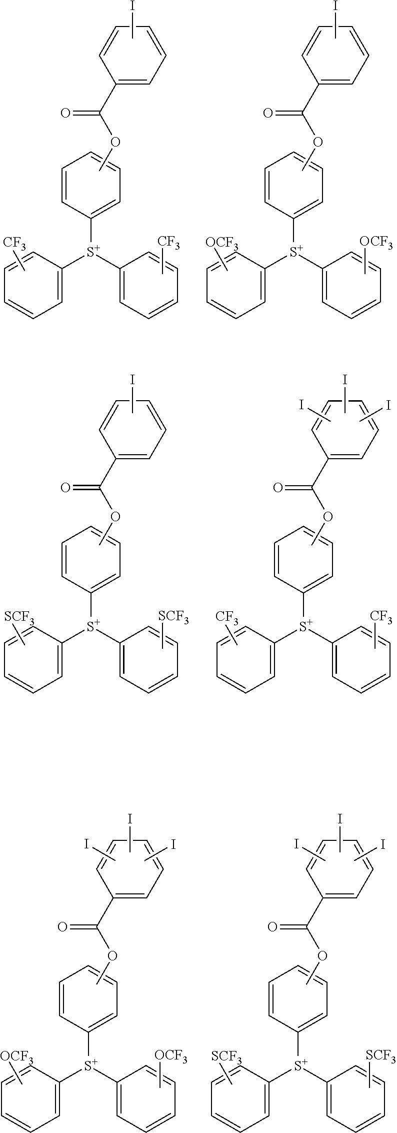

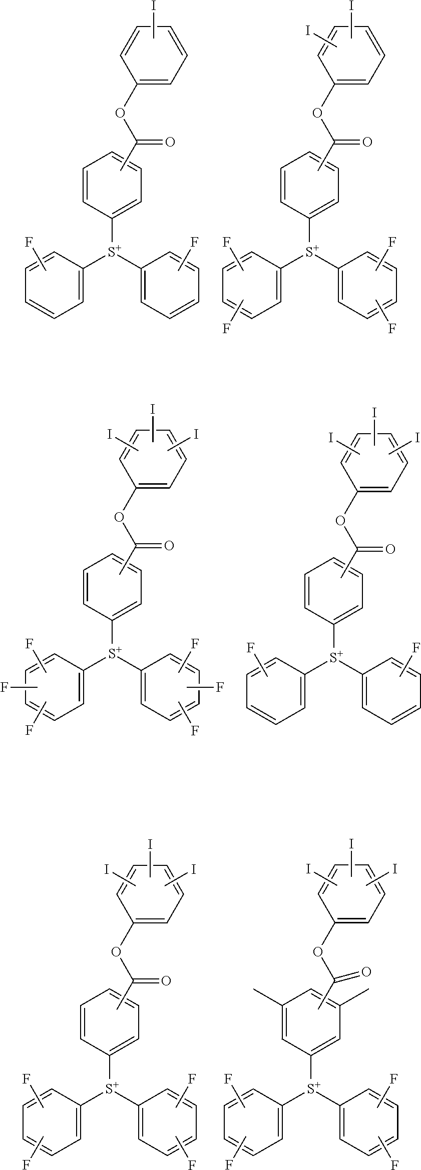

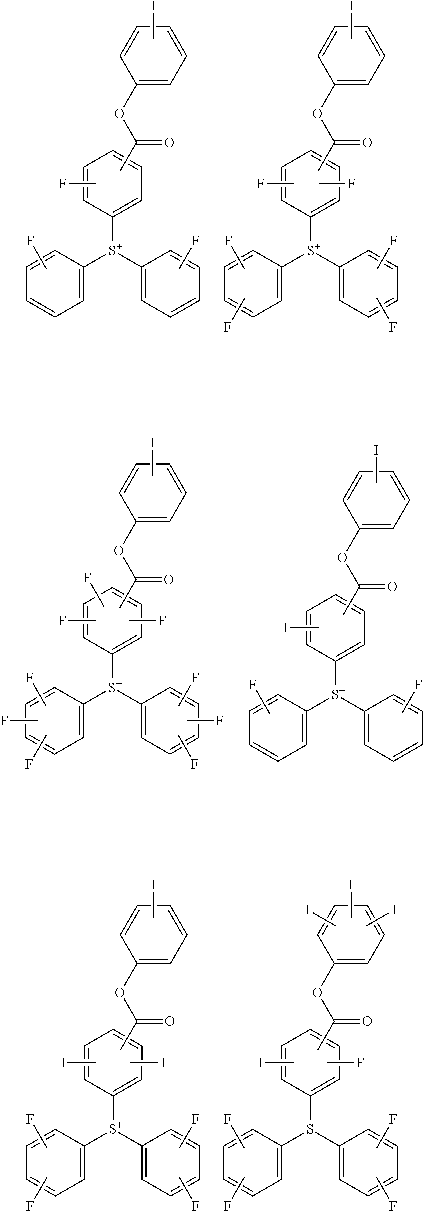

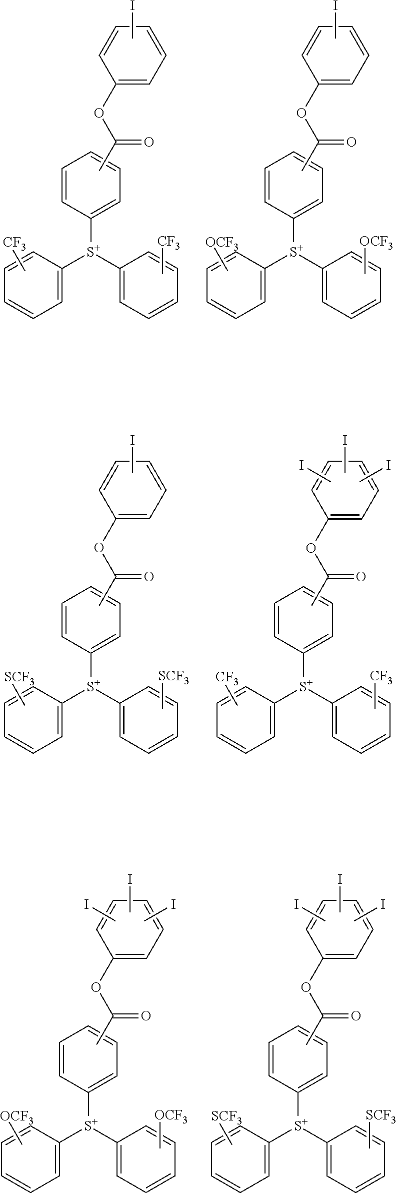

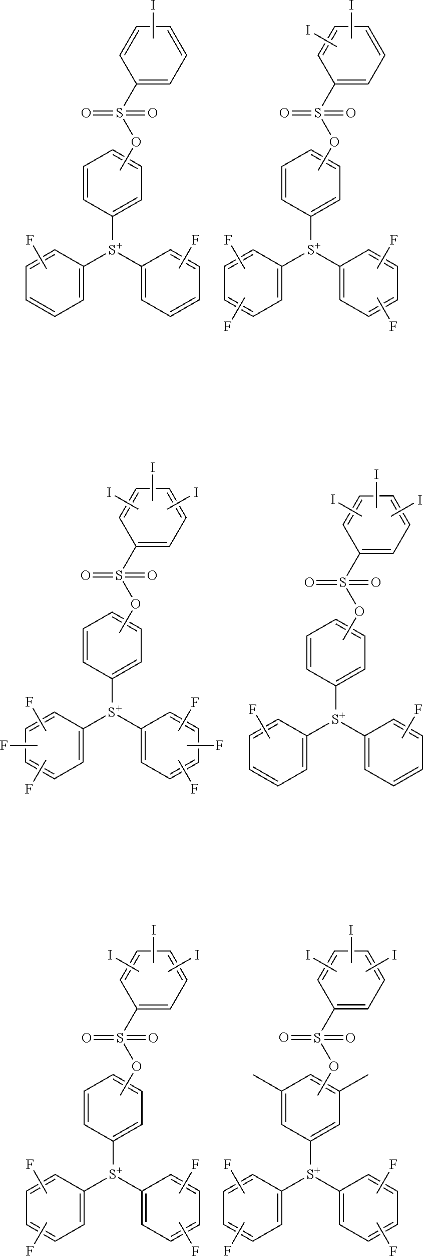

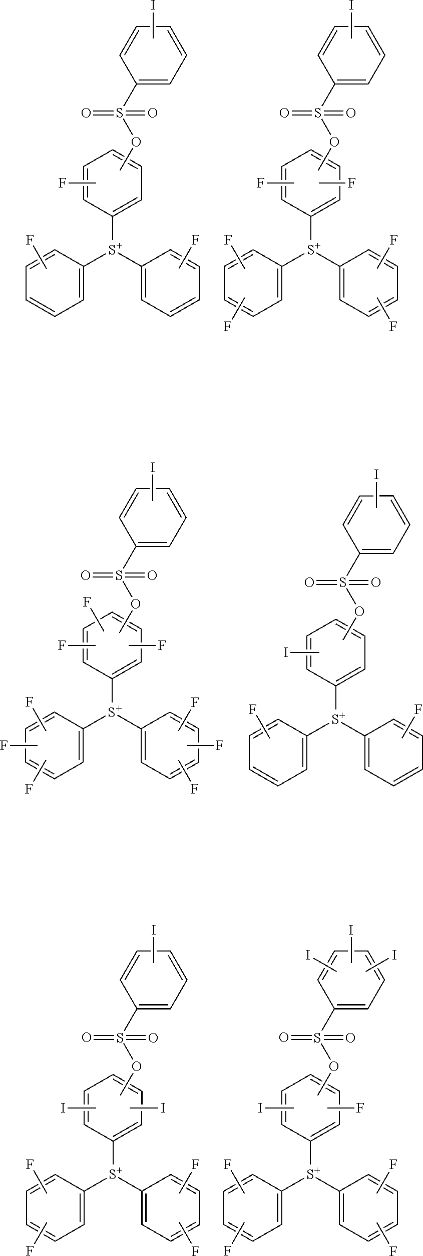

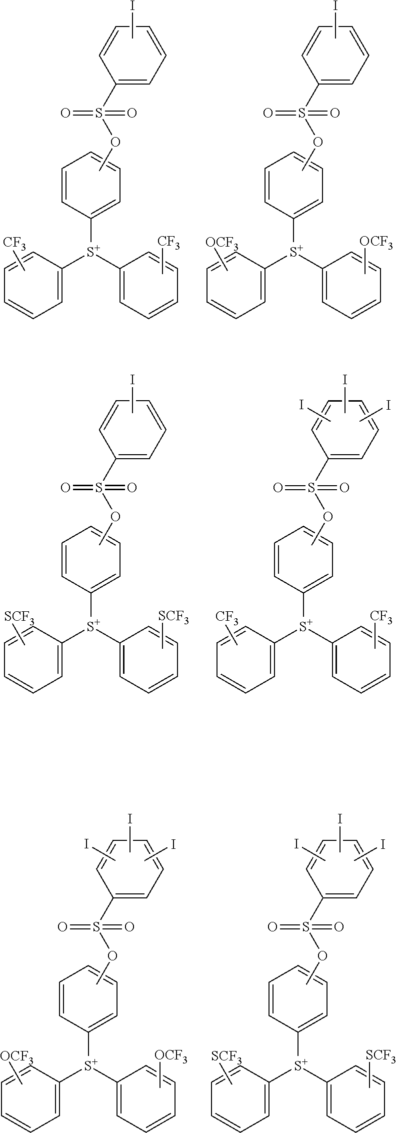

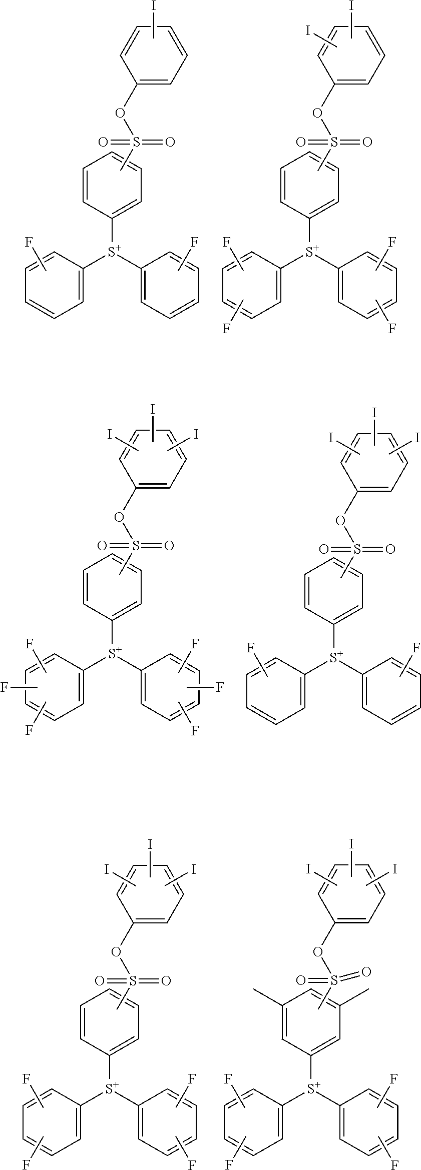

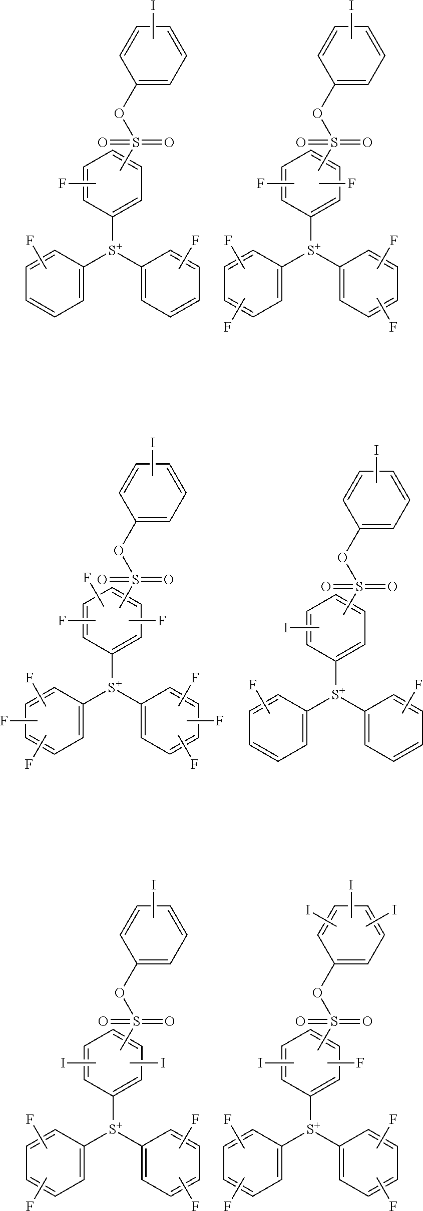

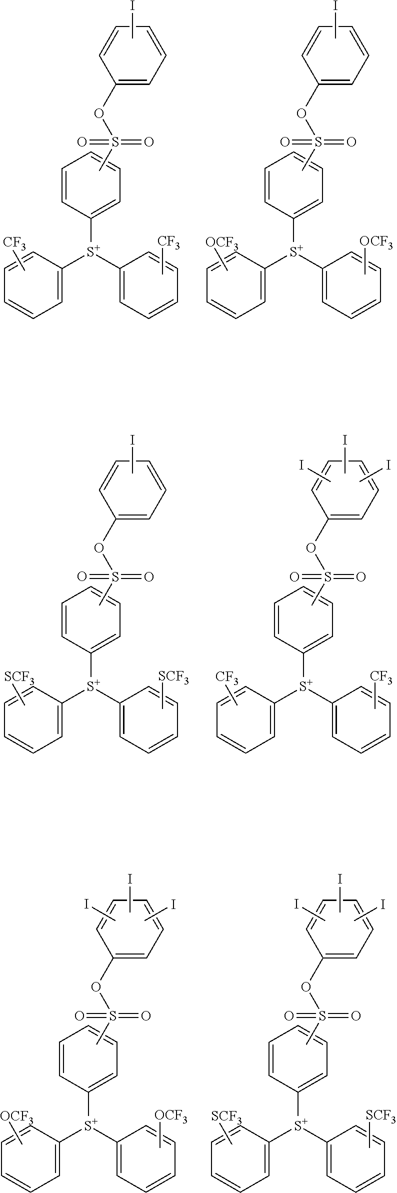

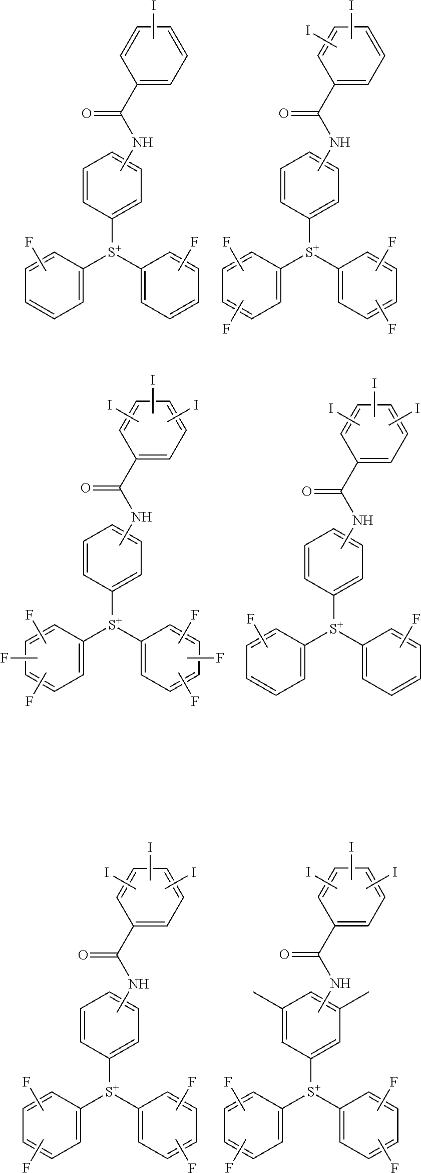

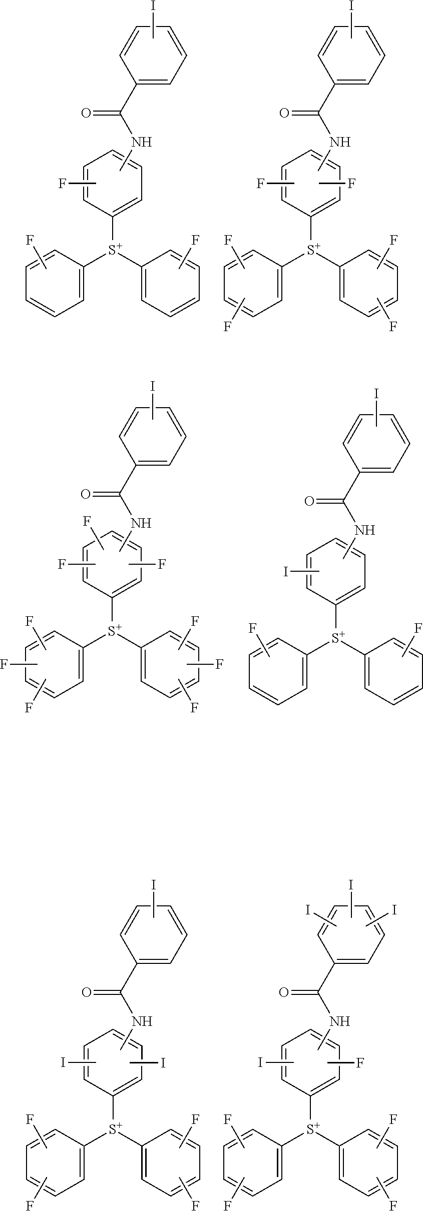

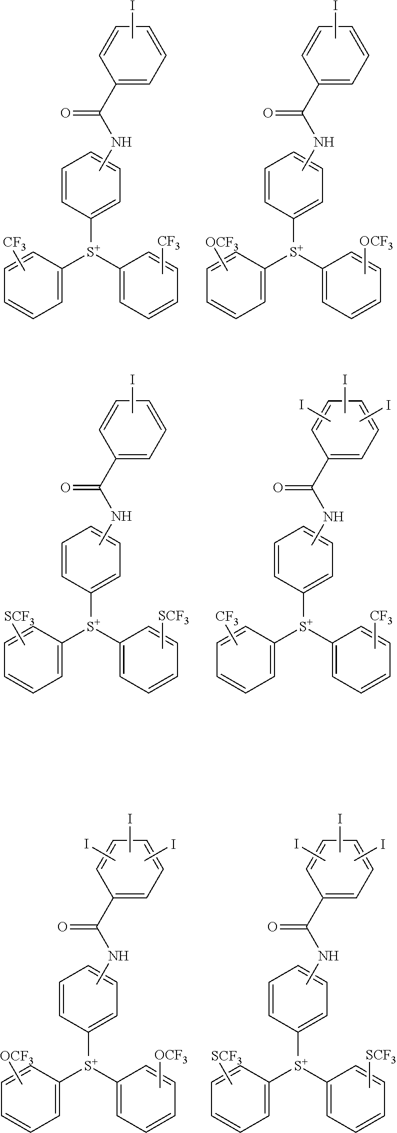

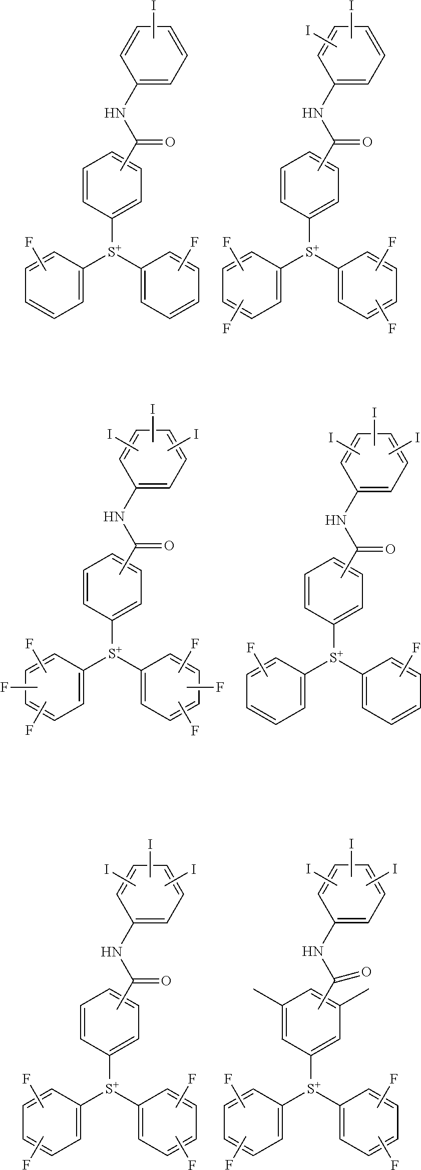

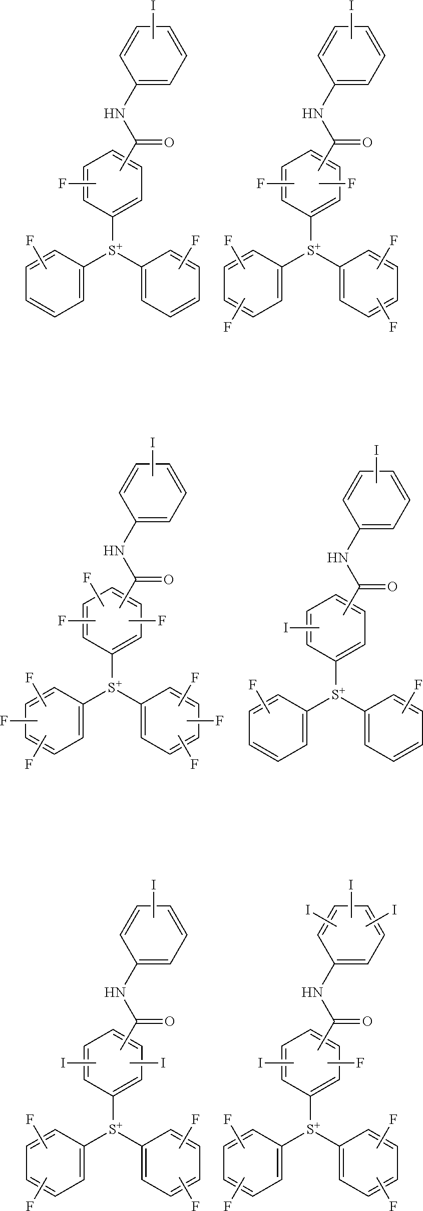

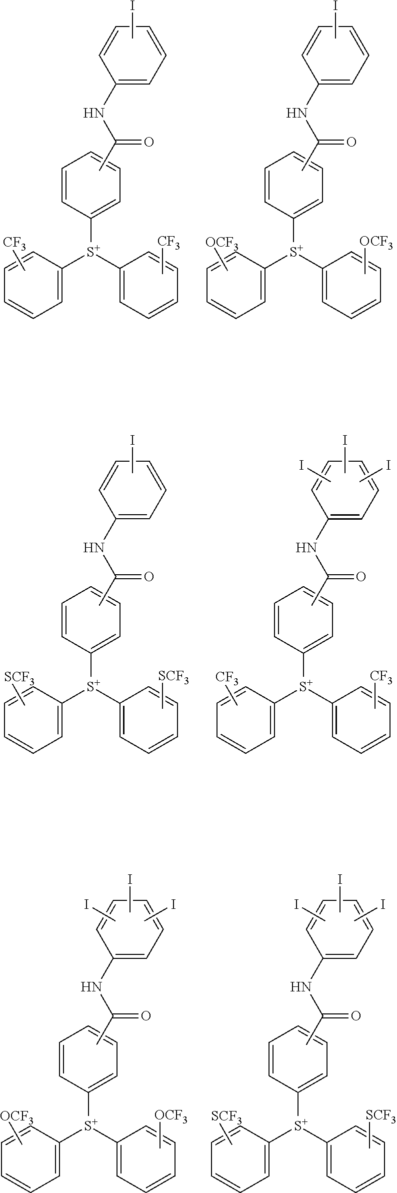

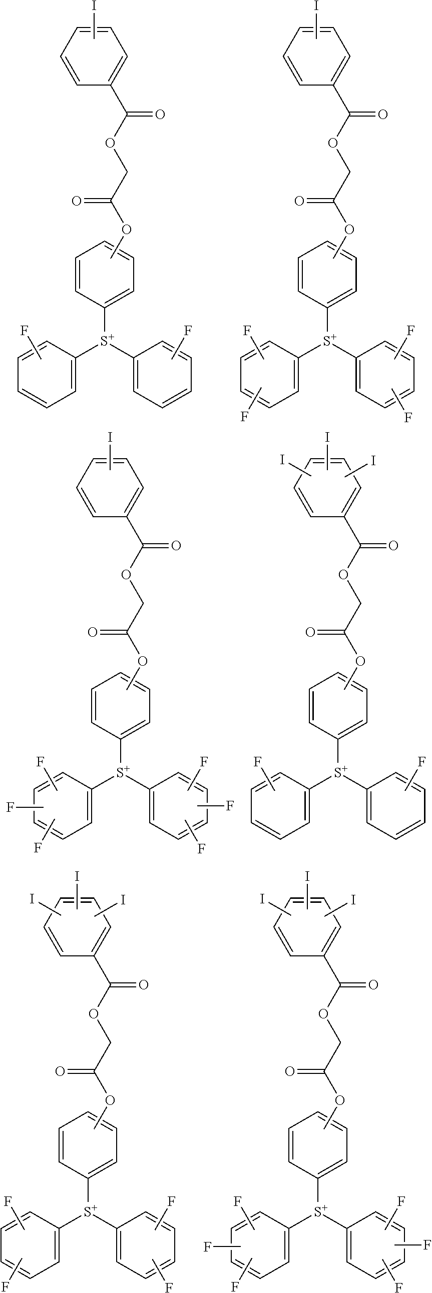

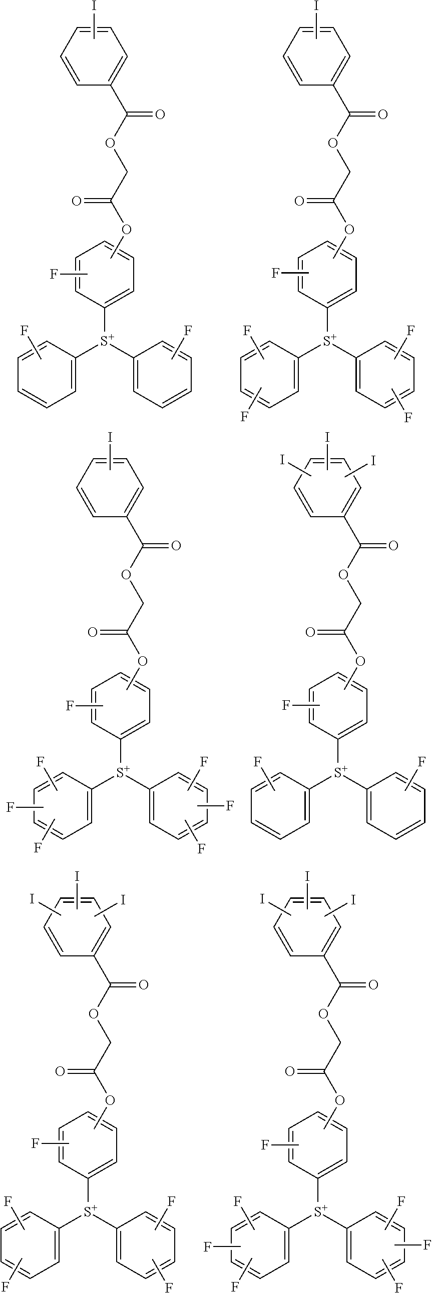

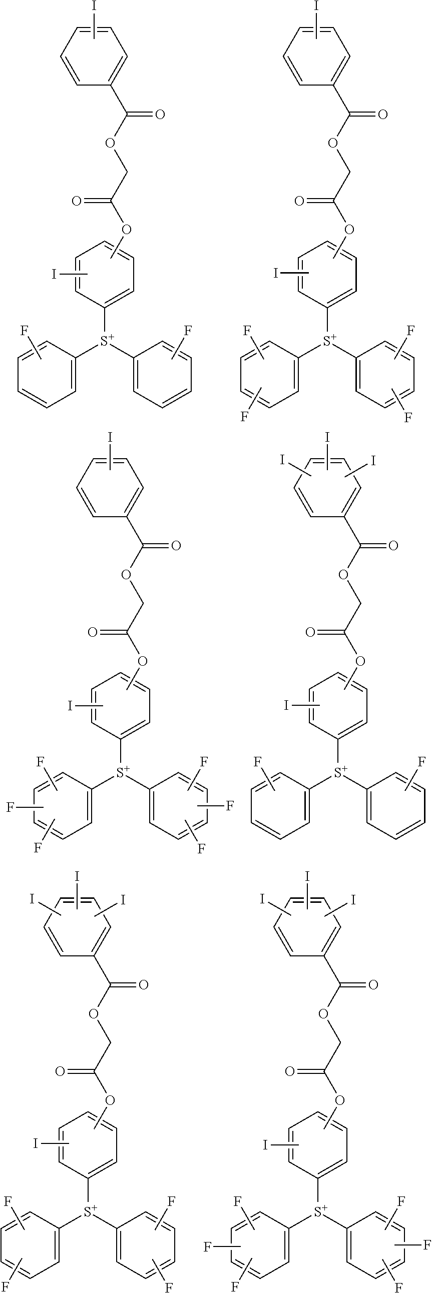

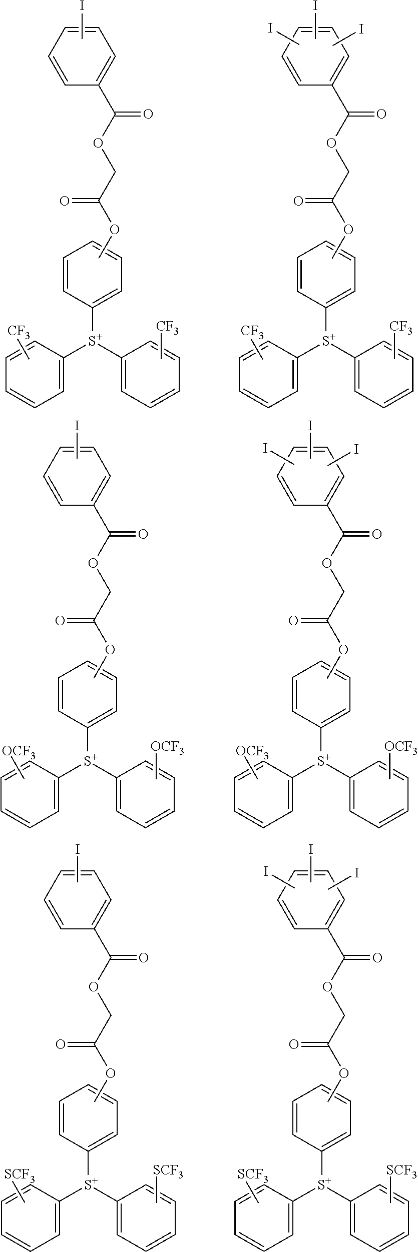

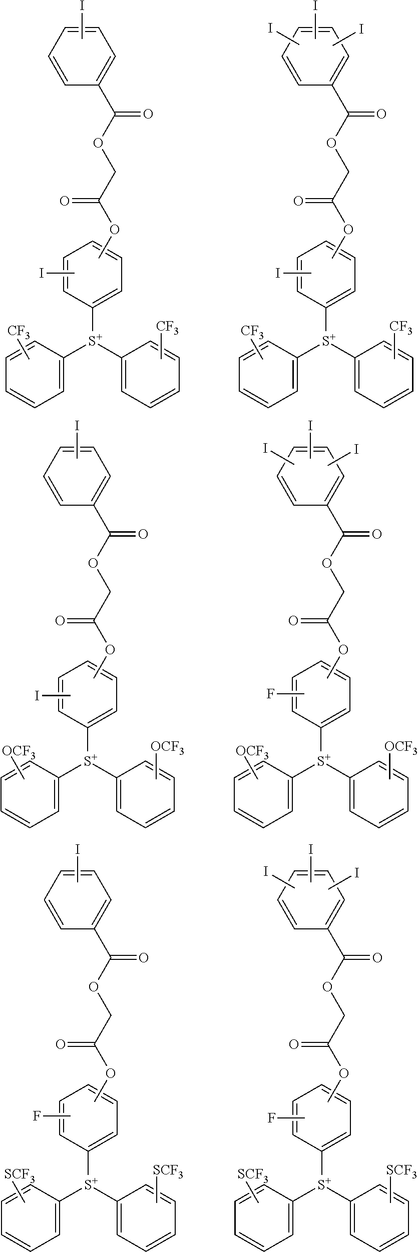

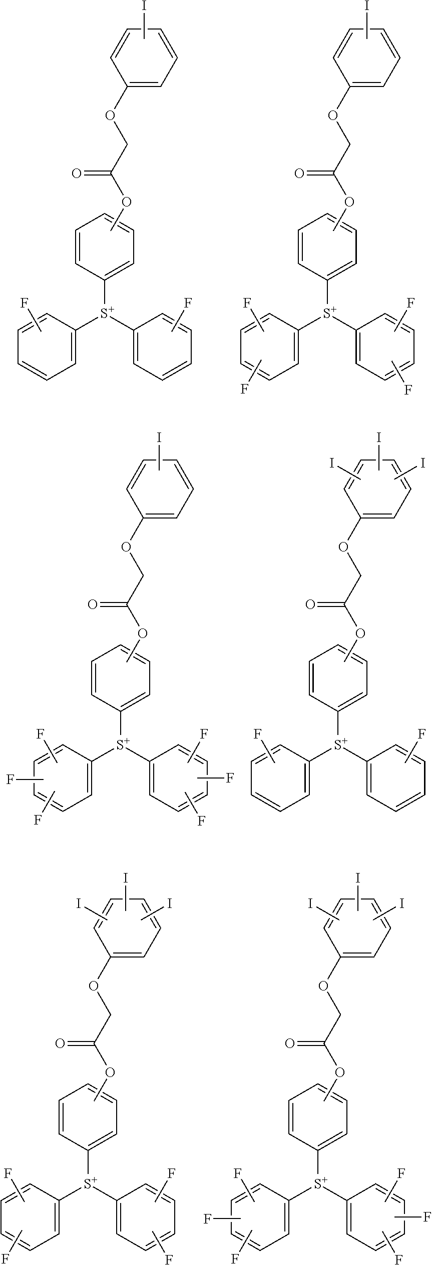

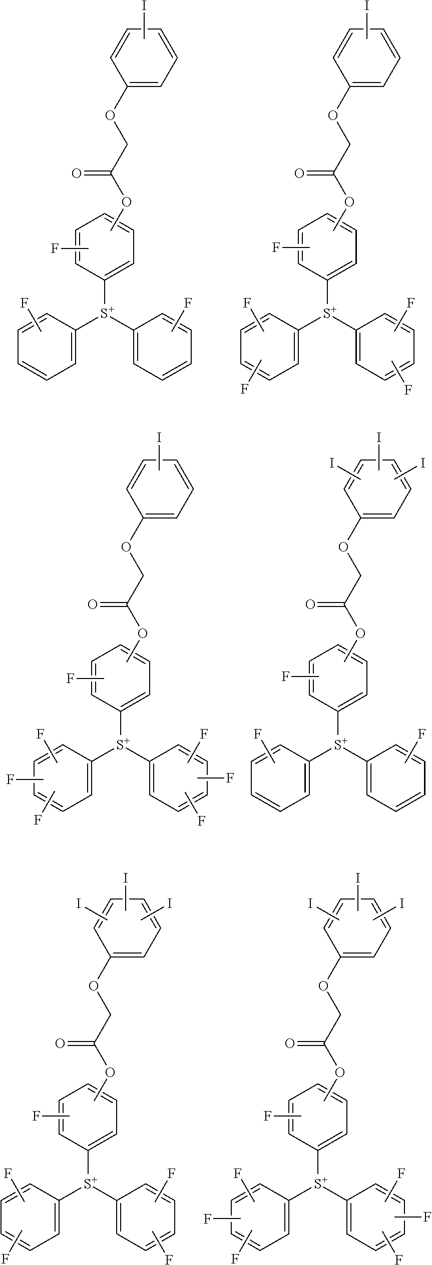

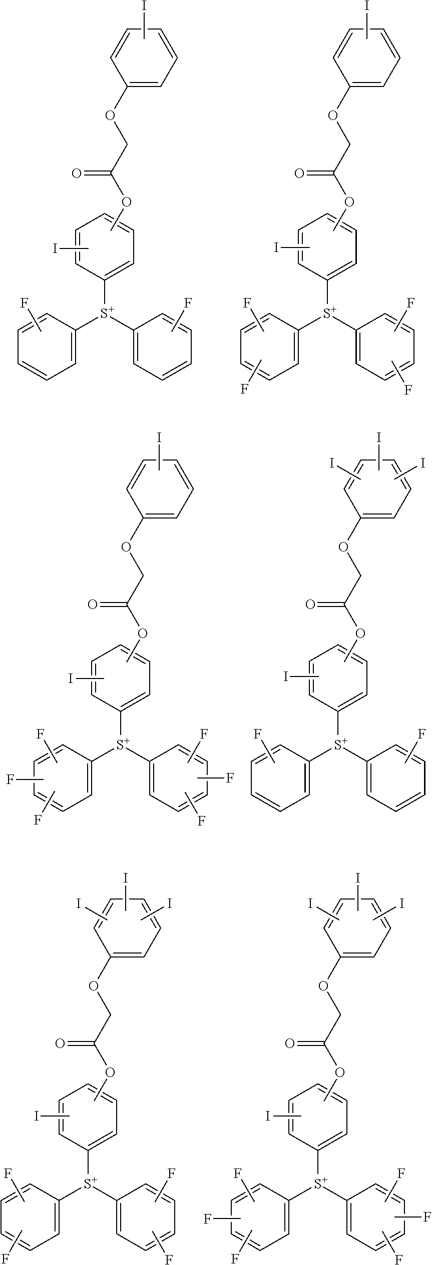

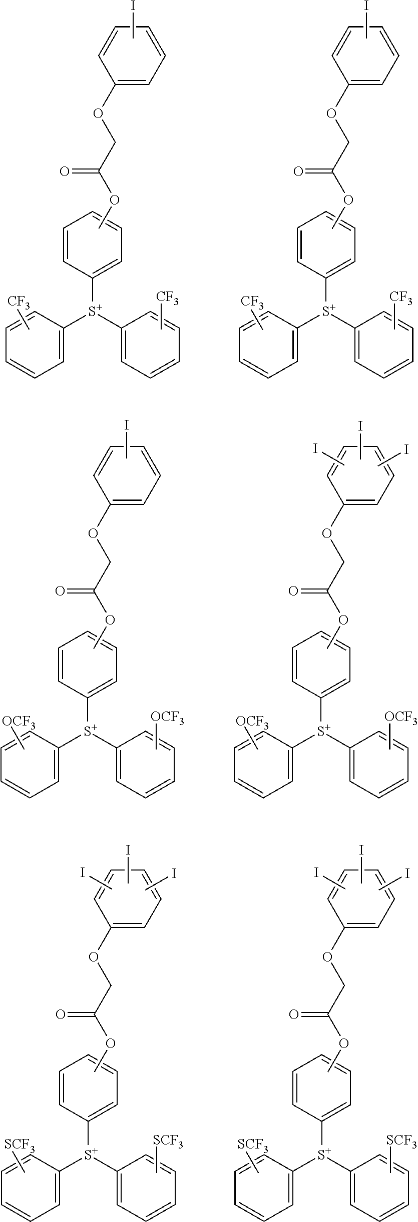

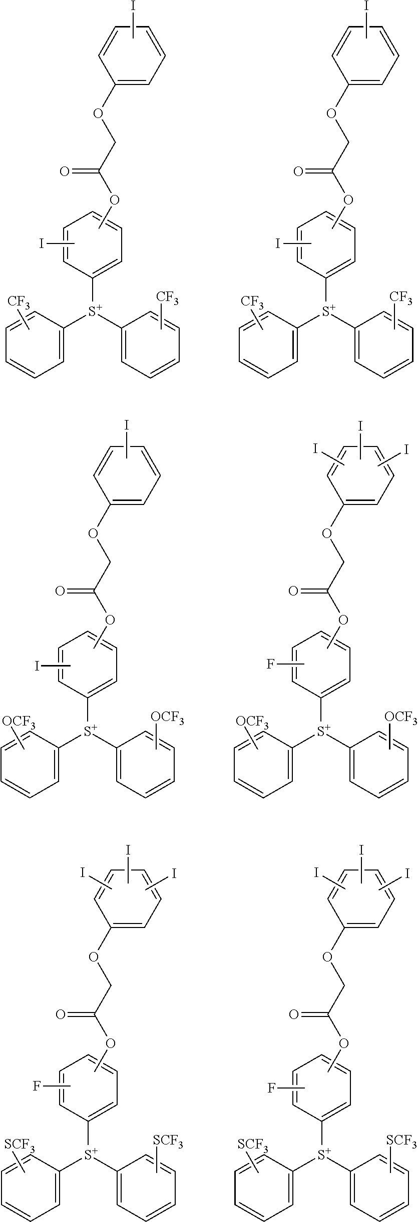

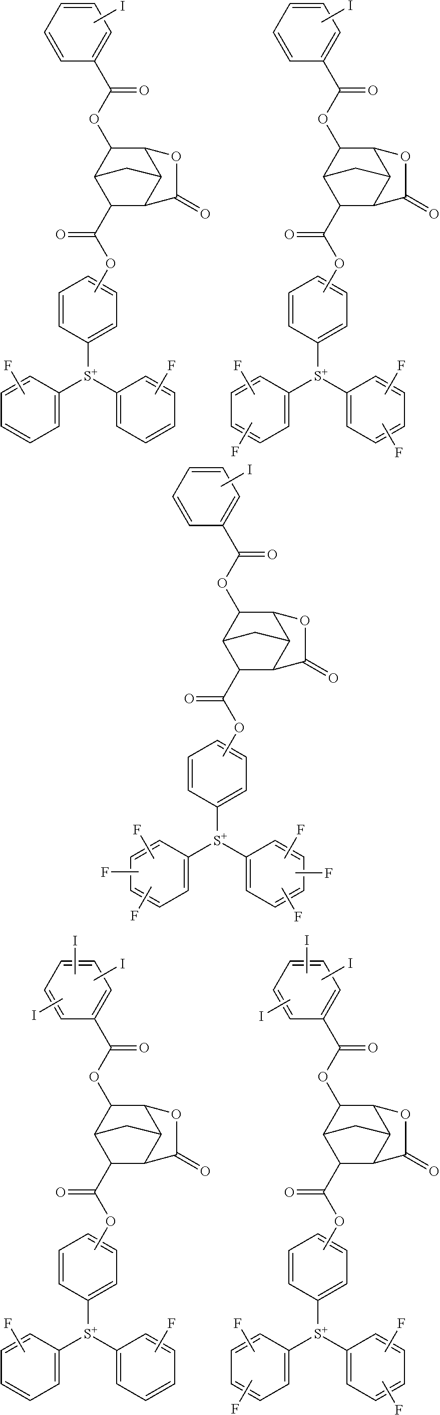

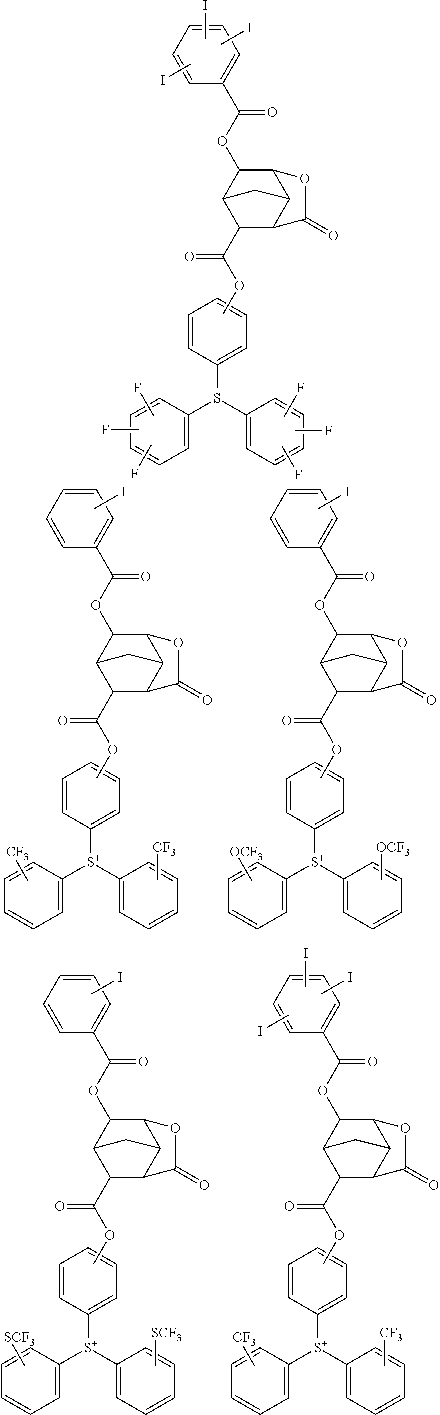

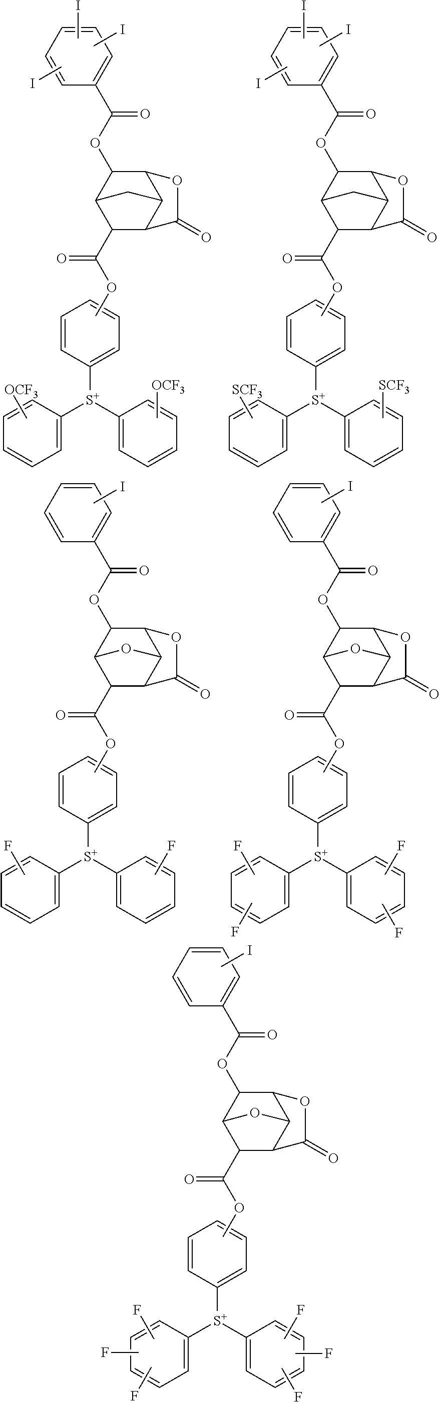

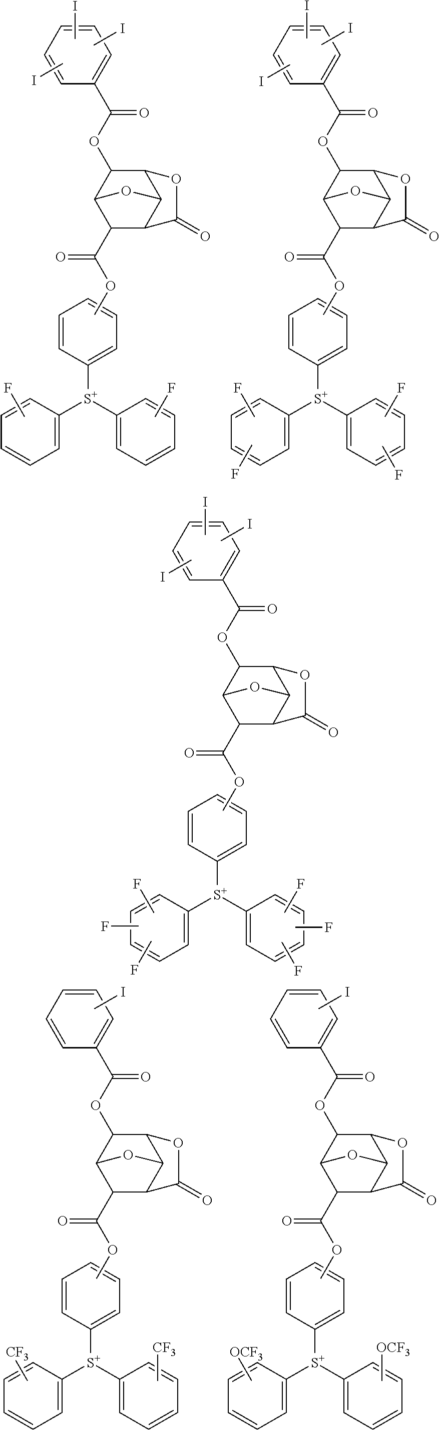

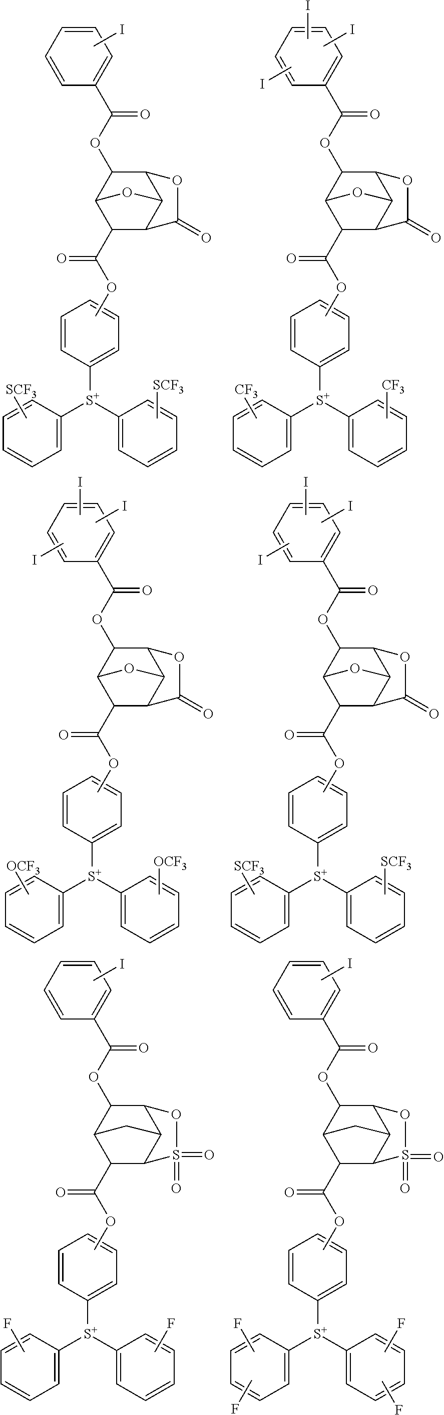

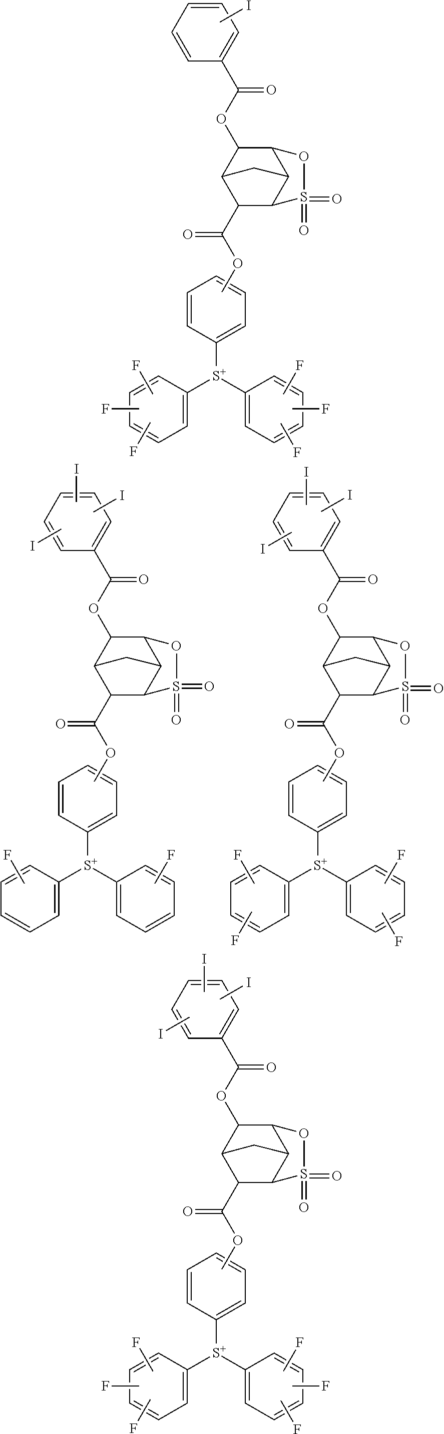

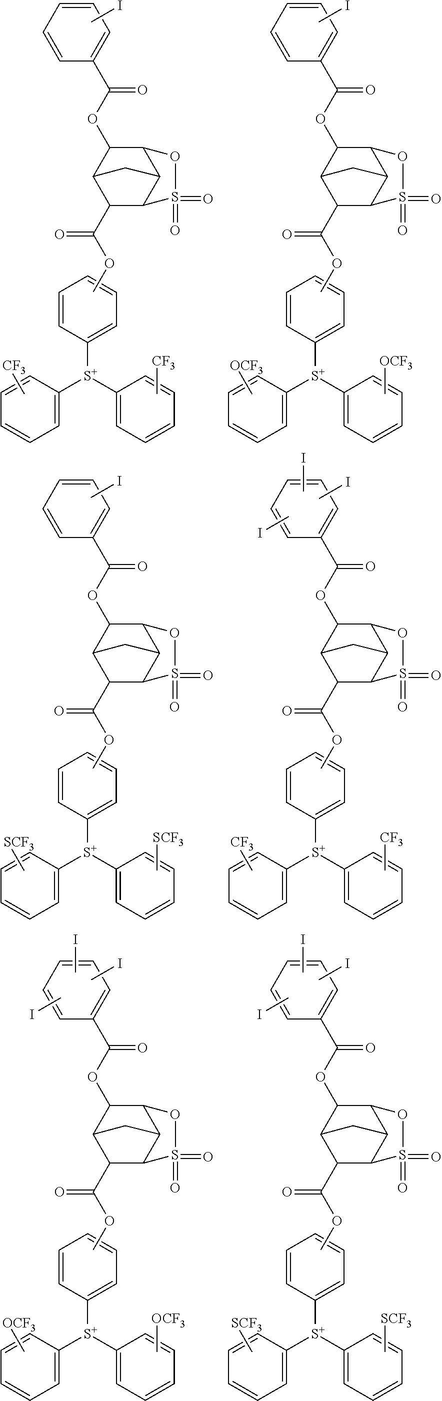

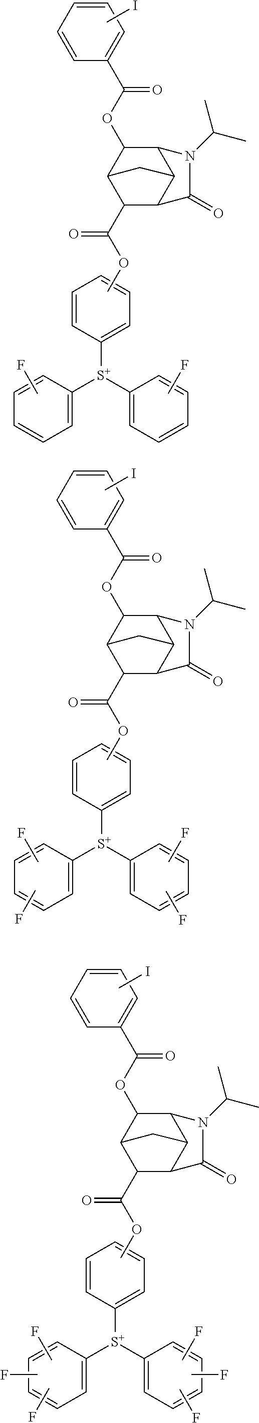

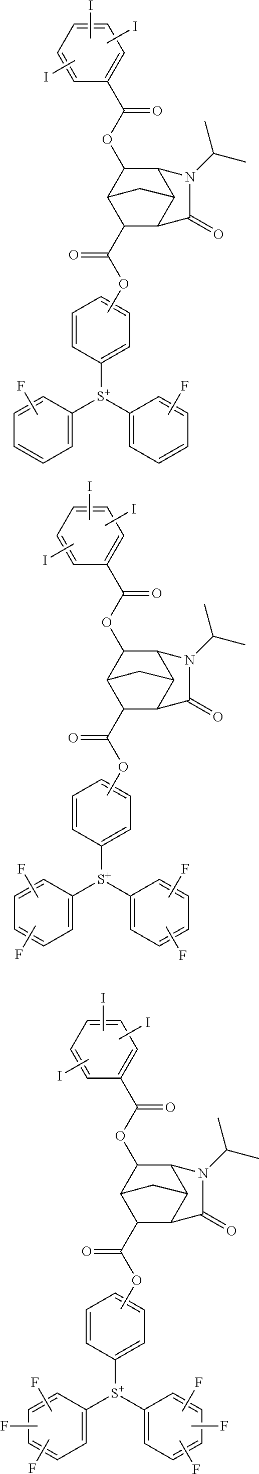

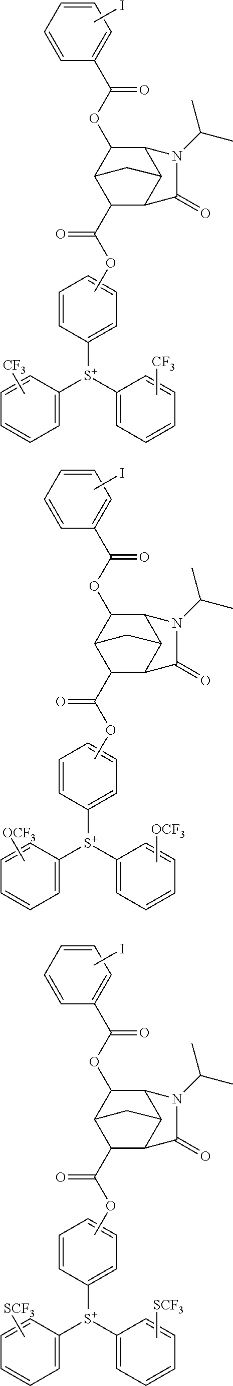

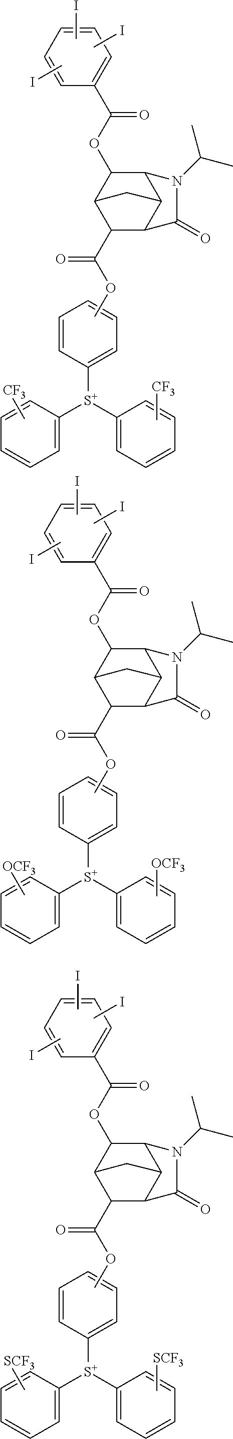

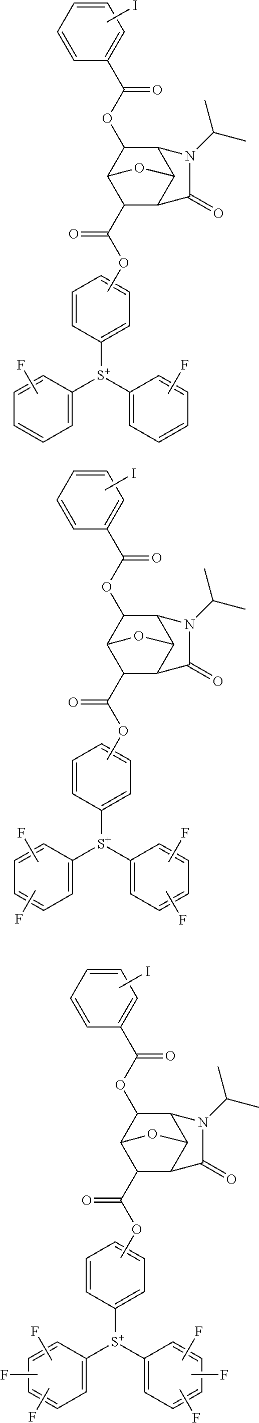

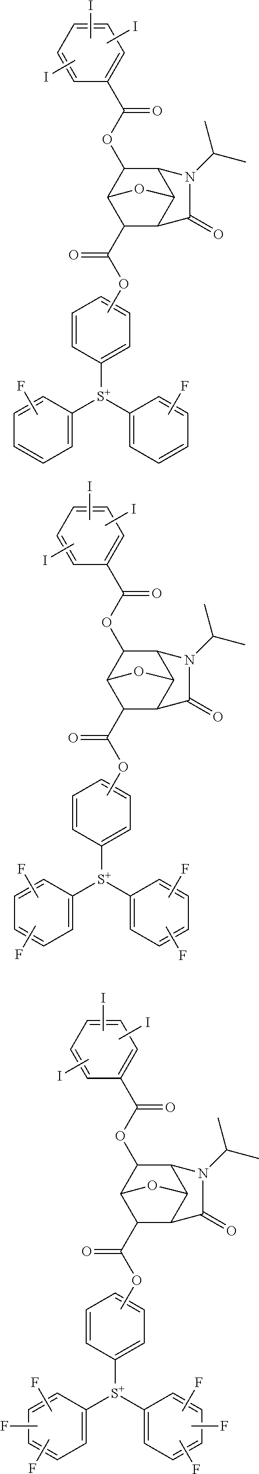

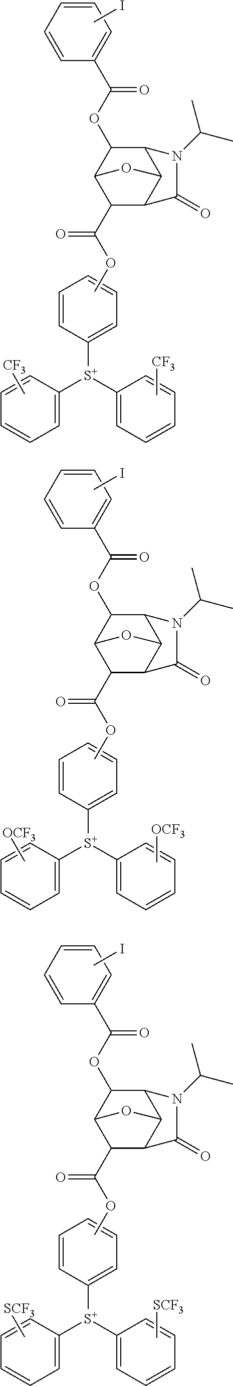

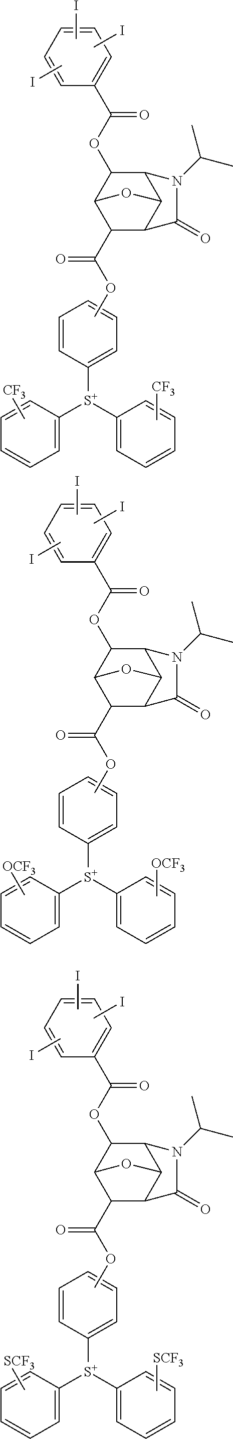

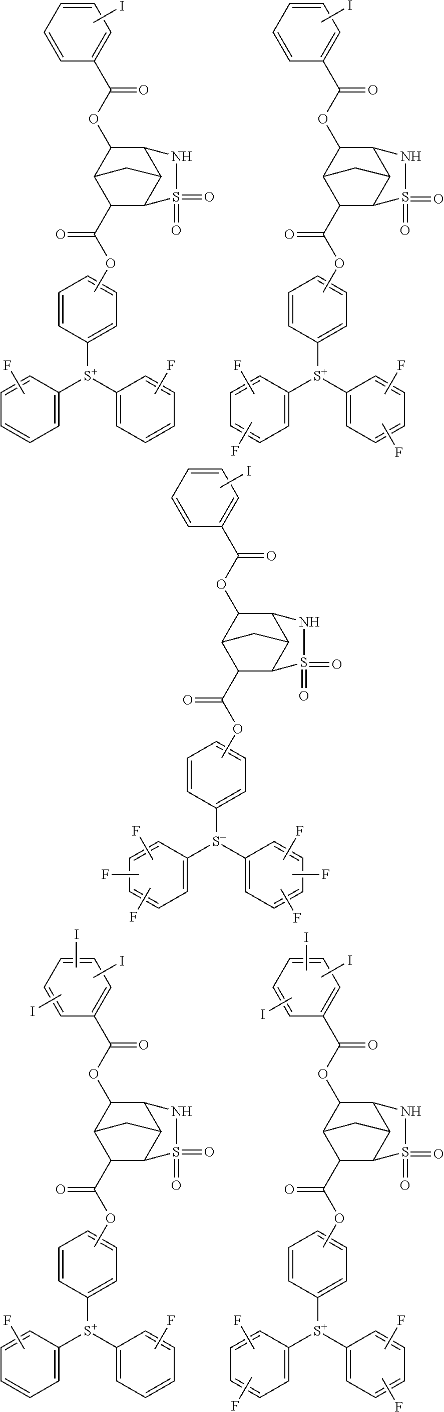

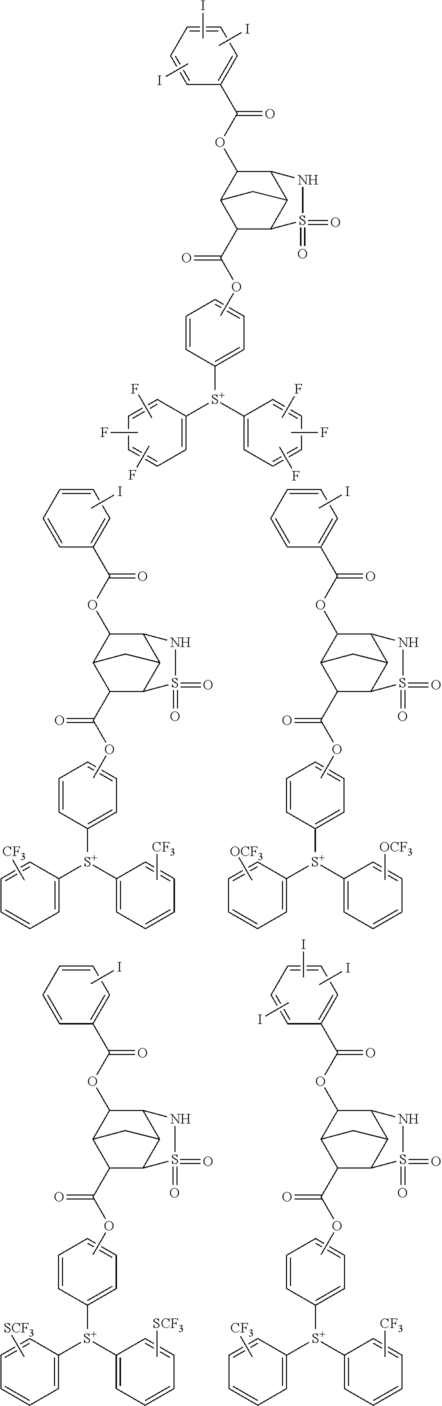

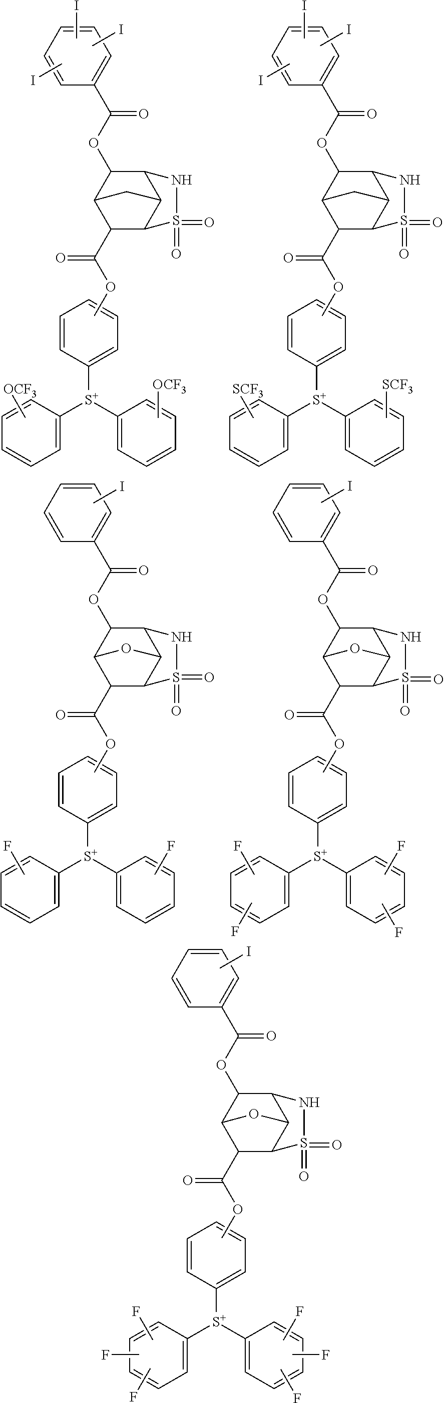

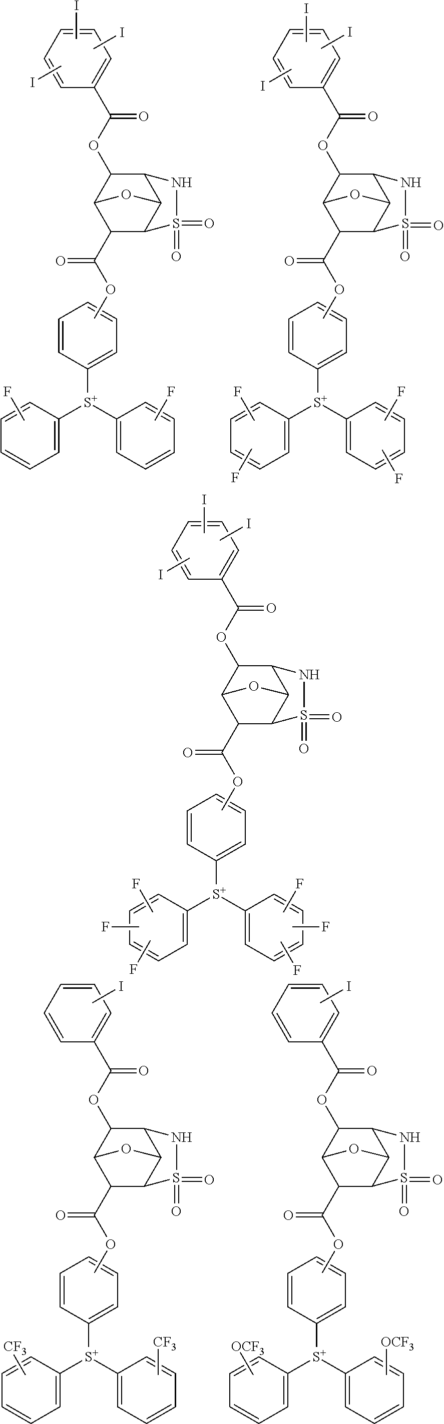

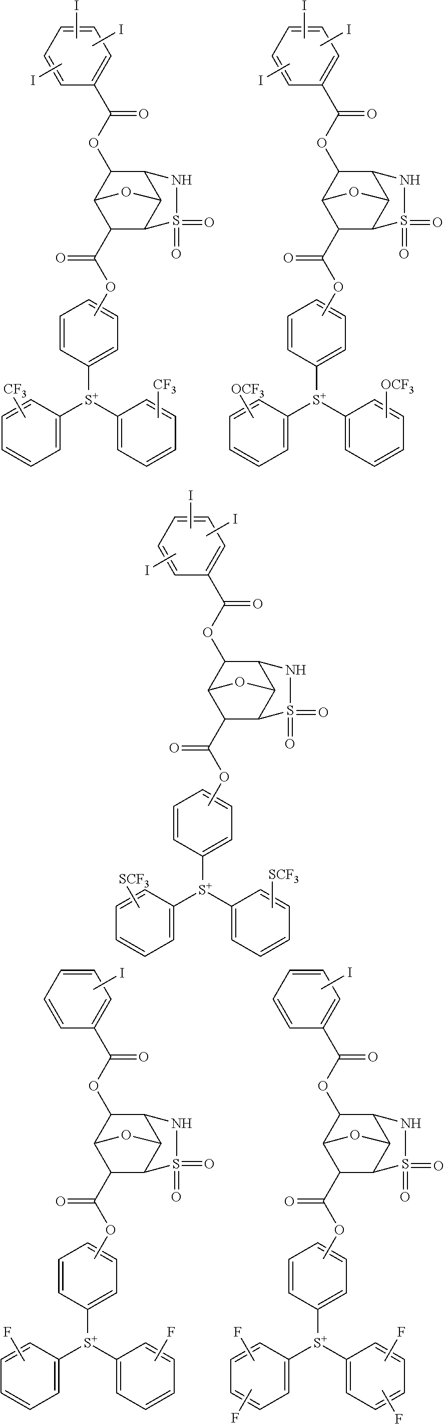

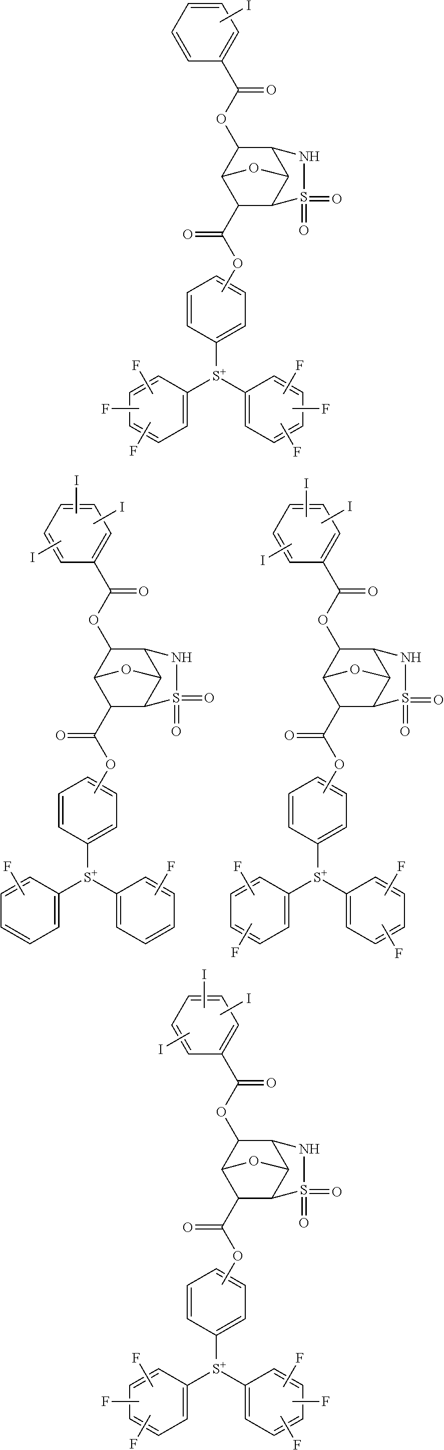

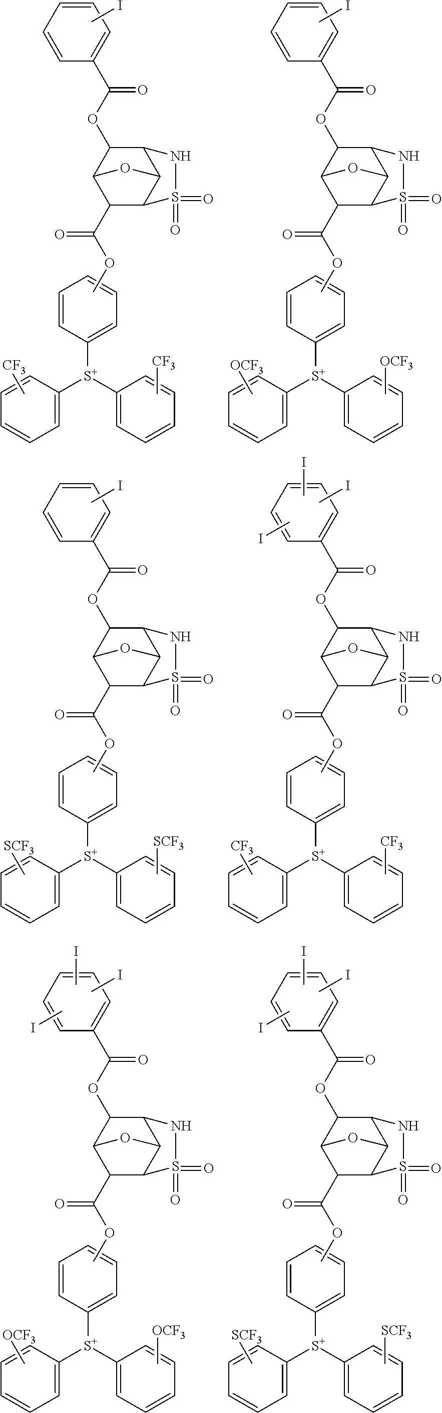

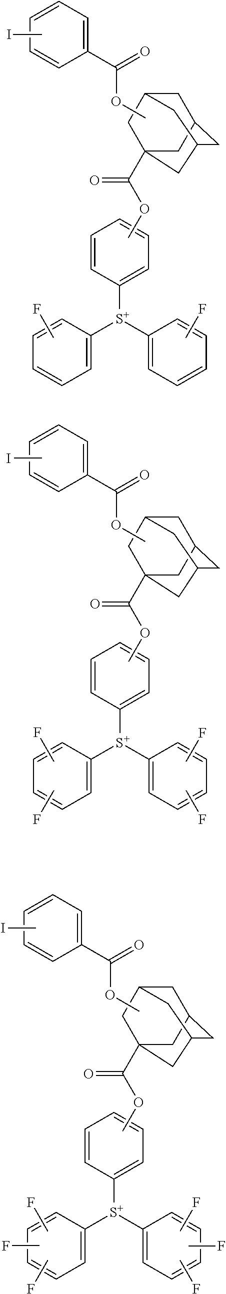

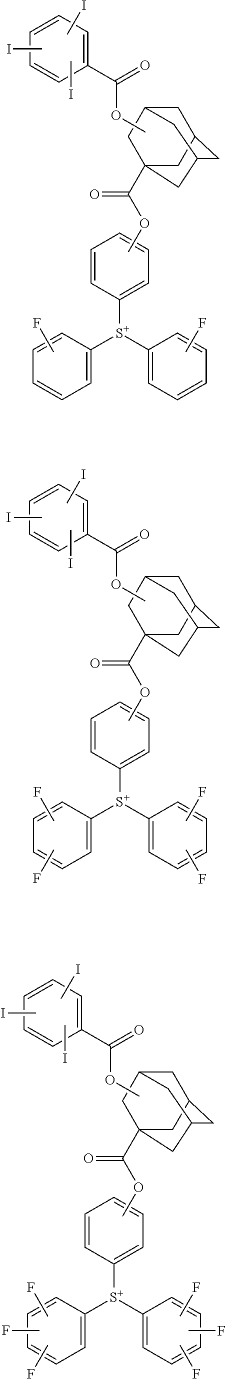

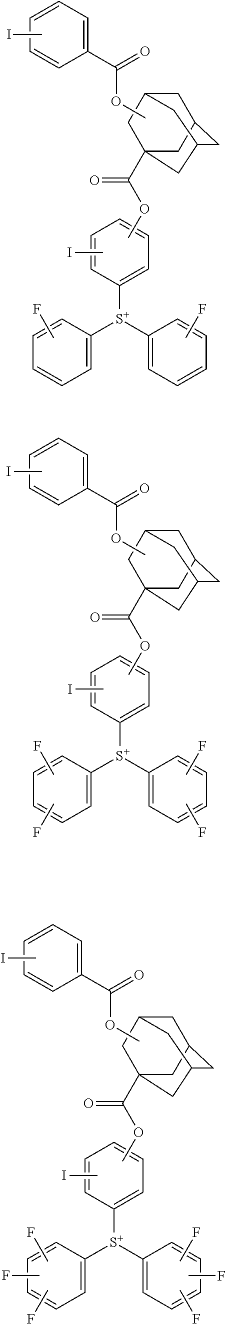

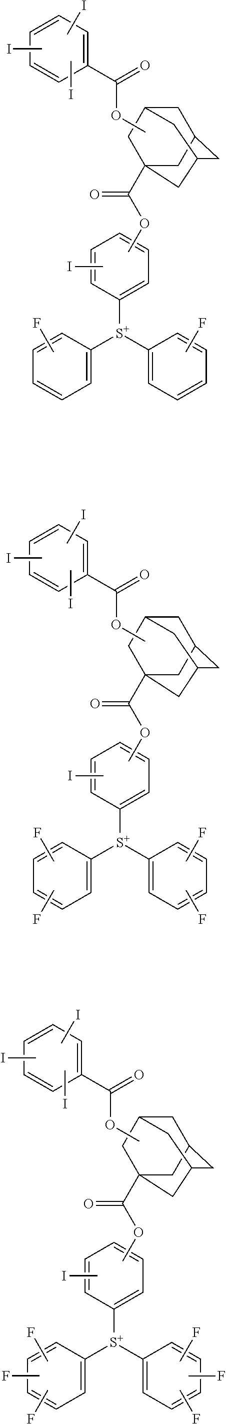

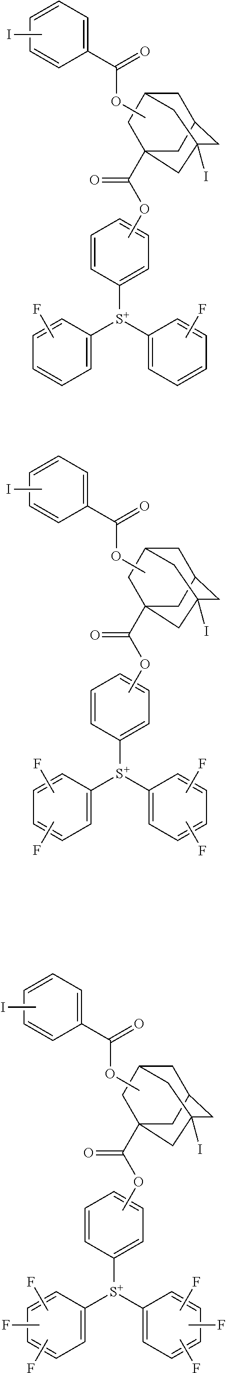

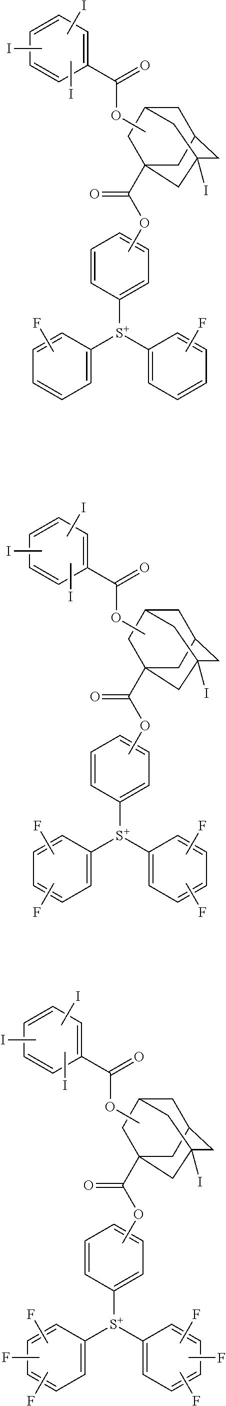

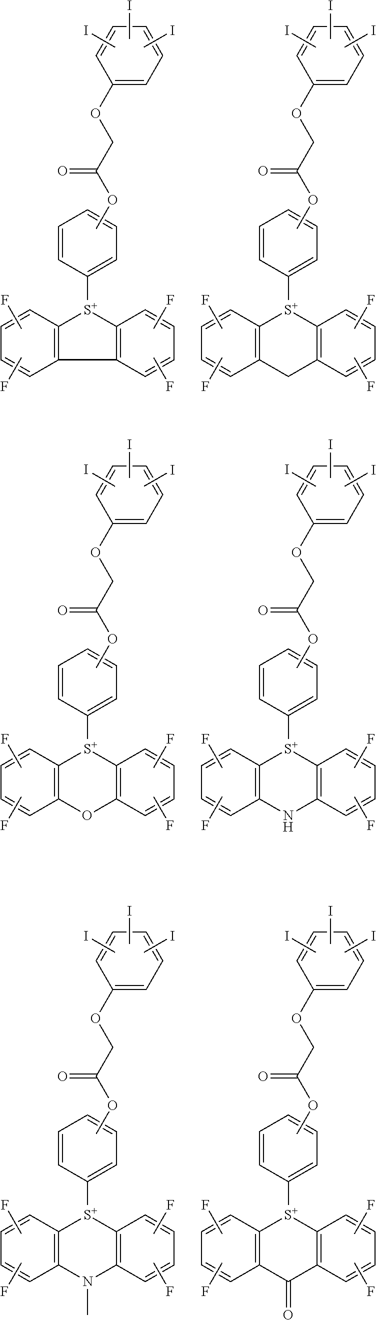

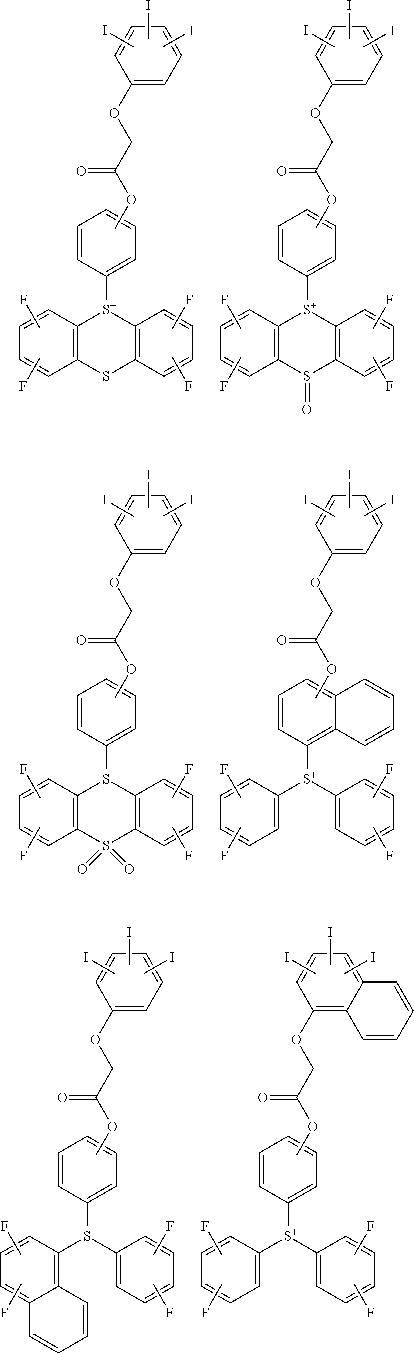

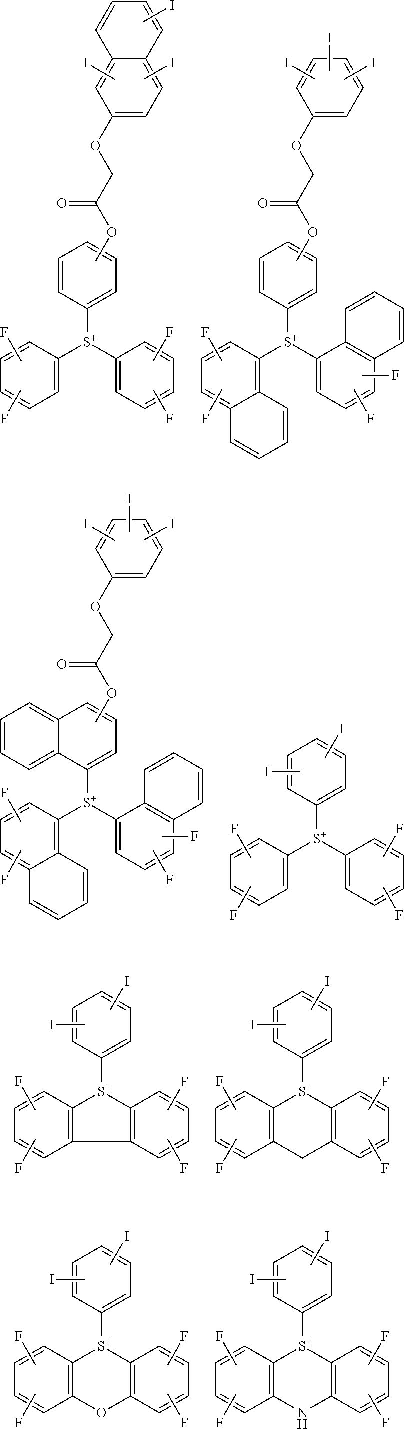

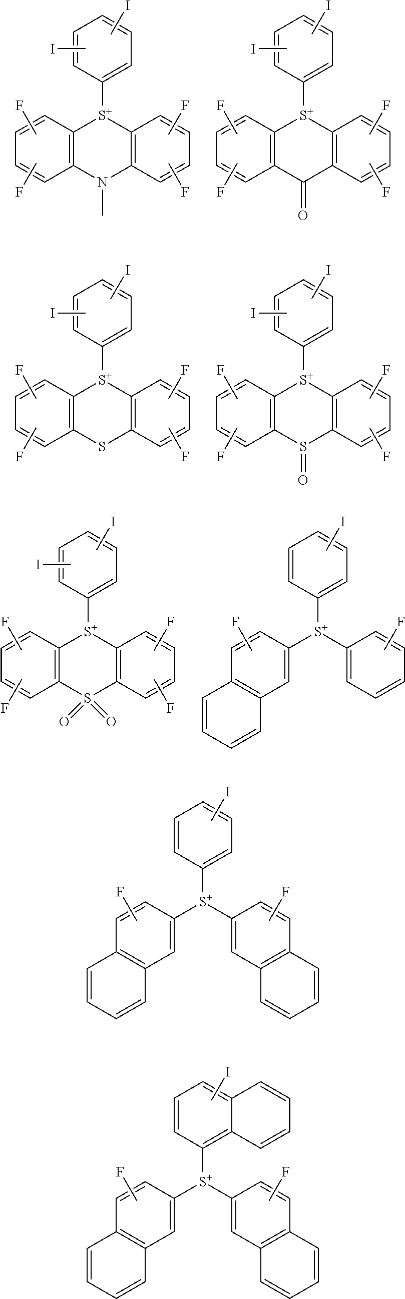

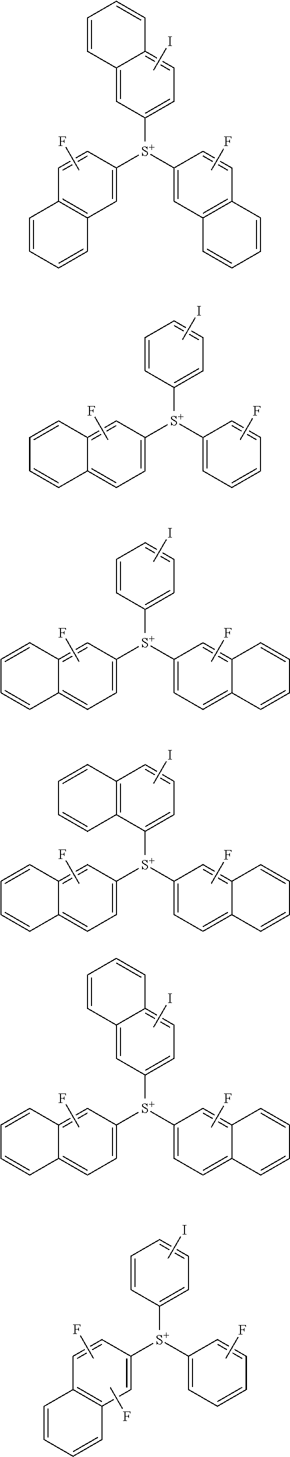

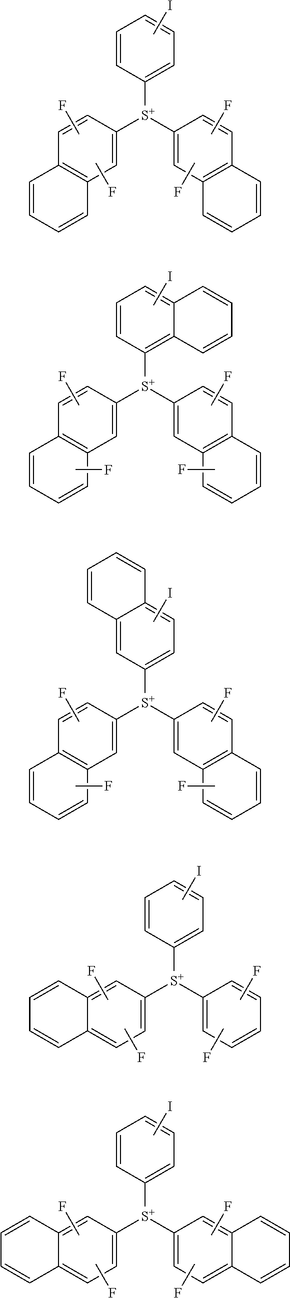

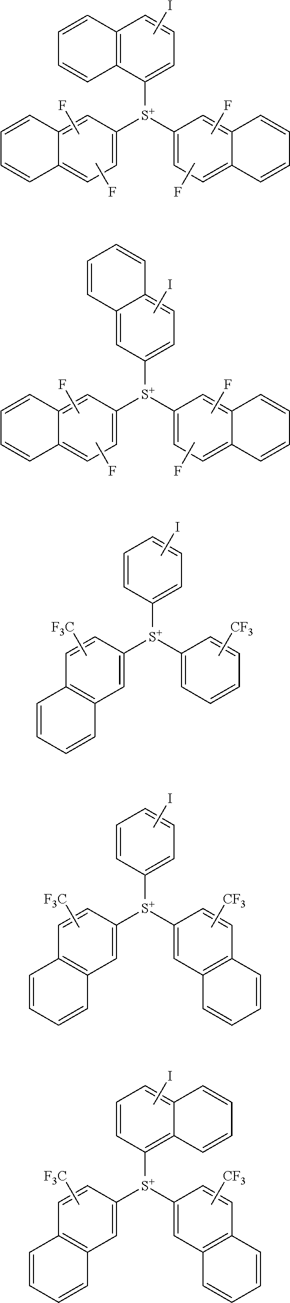

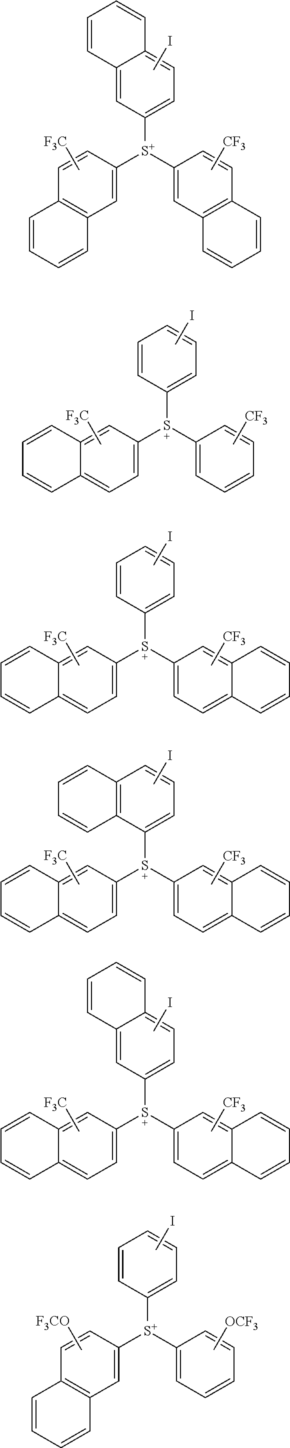

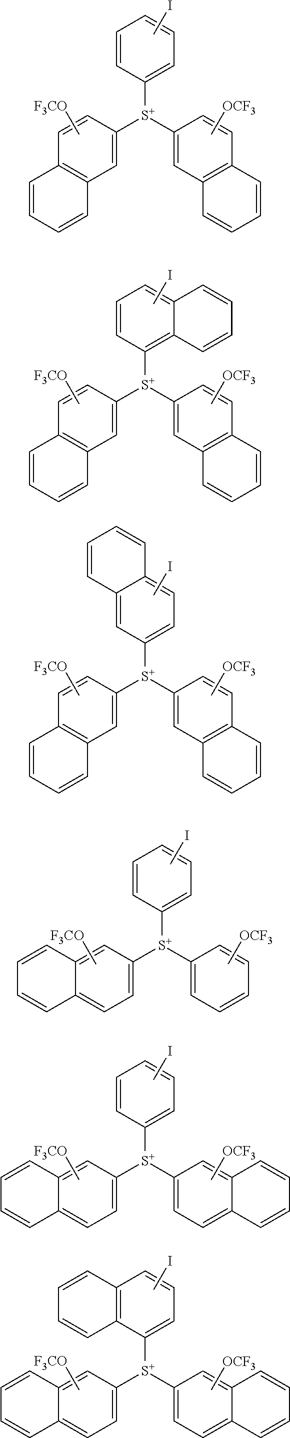

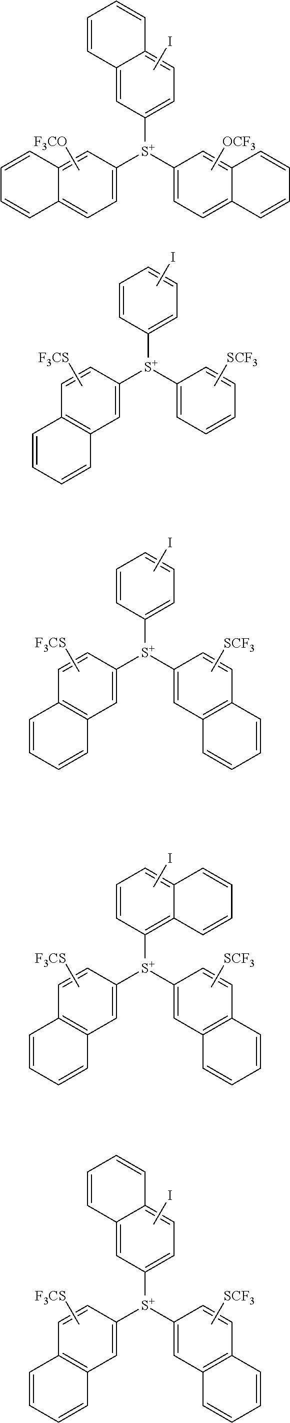

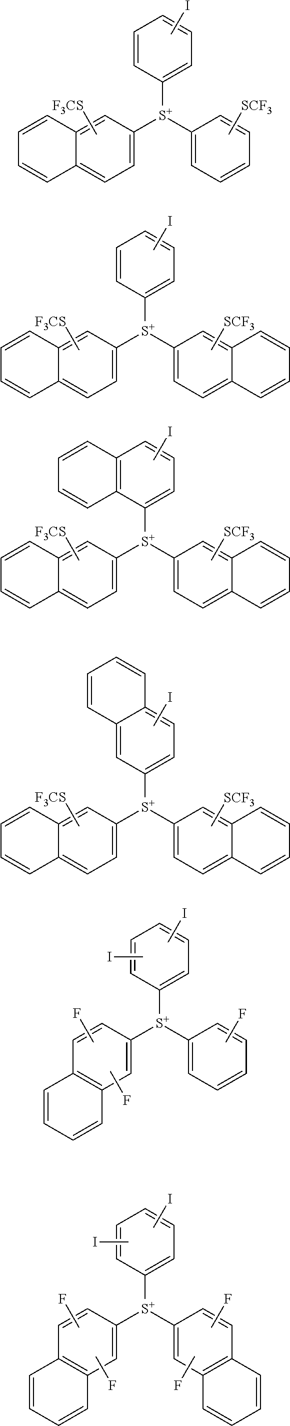

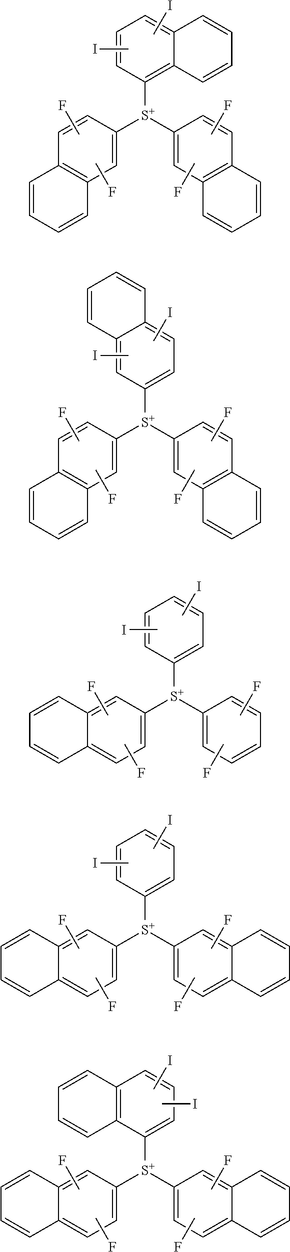

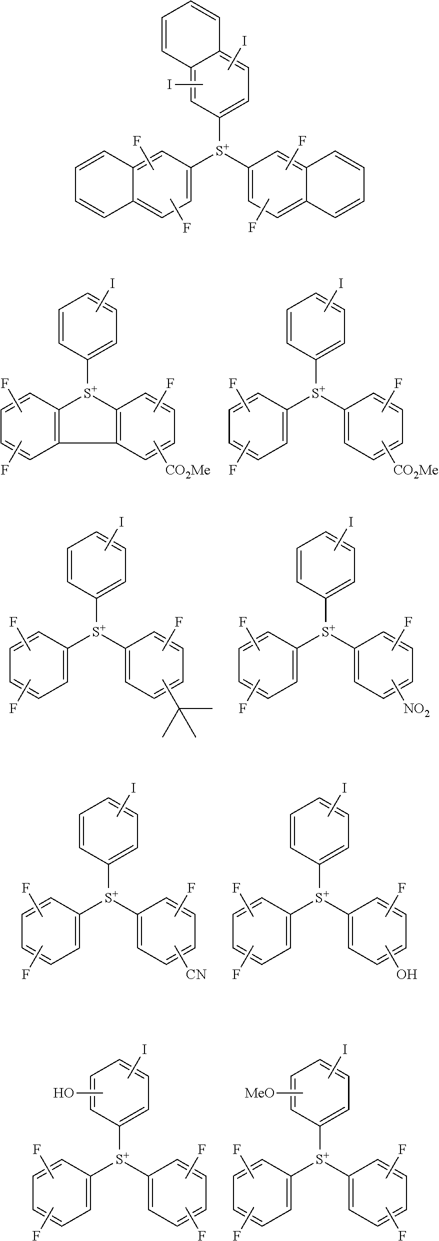

Specific examples of the iodonium cation include, but are not limited to, cations described in paragraph [0181] of JP 2024-259 A.

Specific examples of the ammonium cation include cations represented by formula (am-1):

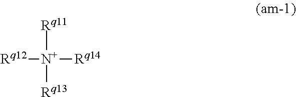

wherein R^{q11} to R^{q14} are each independently a hydrocarbyl group having 1 to 40 carbon atoms and optionally comprising a heteroatom, and R^{q11} and R^{q12} are optionally bonded to each other to form a ring together with a nitrogen atom to which the R^{q11} and R^{q12} are bonded. Specific examples of the hydrocarbyl group include the same hydrocarbyl group represented by R¹ in the description of formula (A) as illustrated.

Specific examples of the ammonium cation represented by formula (am-1) are given below, but not limited thereto.

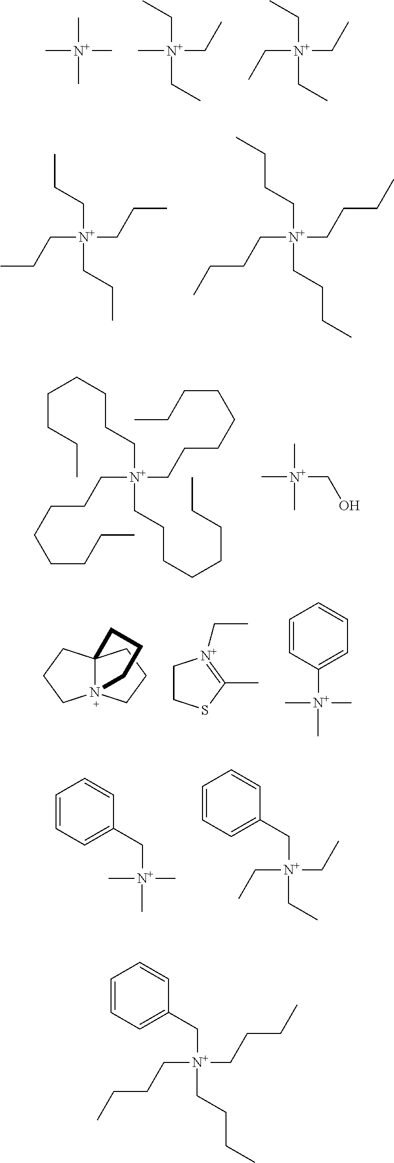

Specific structures of the repeating units c1 to c5 include any combination of the anions and cations described above.

Of the repeating units c1 to c5, the repeating units c2 to c5 are preferable from the viewpoint of acid diffusion control, the repeating units c2, c4, and c5 are further preferable from the viewpoint of acid strength of the generated acid, and the repeating unit c2 is more preferable from the viewpoint of solvent solubility.

The base polymer may further contain a repeating unit having a structure of a hydroxy group protected with an acid labile group (hereinafter referred to as repeating unit d). The repeating unit d is not particularly limited as long as the unit has one or more structures with protected hydroxy groups in which a hydroxyl group is resumed as a result of decomposition of the protective group under the action of acid.

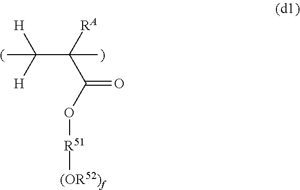

In formula (d1), R^{A} is the same as above; R⁵¹ is an (f+1)-valent hydrocarbon group having 1 to 30 carbon atoms and optionally comprising a heteroatom; R⁵² is an acid labile group; and f is an integer of 1 to 4.

In formula (d1), the acid labile group represented by R⁵² may be any group that is deprotected under the action of acid to generate a hydroxy group. The structure of R⁵² is not particularly limited, but is preferably, for instance, an acetal structure, a ketal structure, an alkoxycarbonyl group, or an alkoxymethyl group represented by formula (d2), and particularly preferably an alkoxymethyl group represented by formula (d2):

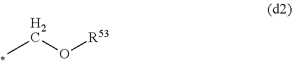

wherein “*” represents a bond, and R⁵³ is a hydrocarbyl group having 1 to 15 carbon atoms.

Specific examples of the acid labile group represented by R⁵², the alkoxymethyl group represented by (d2), and the repeating unit d are the same as illustrated in the description of the repeating unit d described in JP 2020-111564 A.

The base polymer may further contain a repeating unit e derived from indene, benzofuran, benzothiophene, acenaphthylene, chromone, coumarin, and norbornadiene, or a derivative thereof. Specific examples of the monomer to yield the repeating unit e are given below, but not limited thereto.

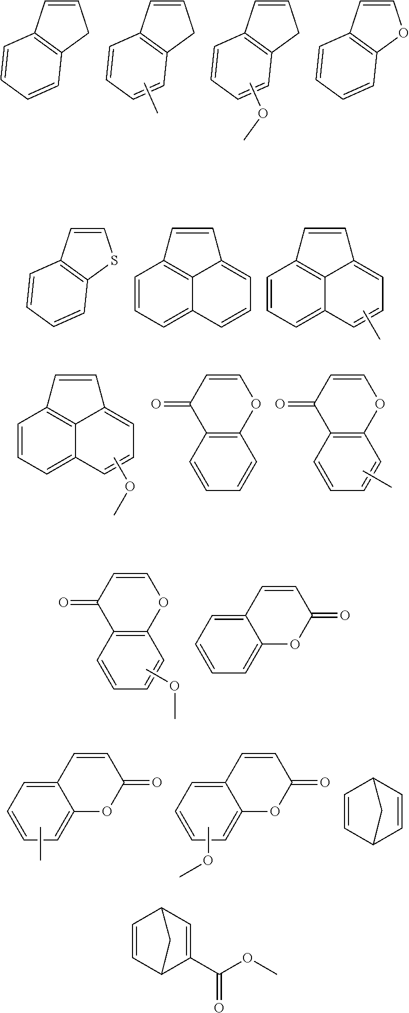

The base polymer may further contain a repeating unit f derived from indane, vinylpyridine, or vinylcarbazole.

In the base polymer, the content ratio of the repeating units a1, a2, a3, b1, b2, c1 to c5, d, e, and f preferably satisfies 0≤a1≤0.8, 0≤a2≤0.8, 0≤a3≤0.6, 0≤b1≤0.6, 0≤b2≤0.6, 0≤c1≤0.4, 0≤c2≤0.4, 0≤c3≤0.4, 0≤c4≤0.4, 0≤c5≤0.4, 0≤d≤0.5, 0≤e≤0.3, and 0≤f≤0.3, and more preferably 0≤a1≤0.7, 0≤a2≤0.7, 0≤a3≤0.5, 0≤b1≤0.5, 0≤b2≤0.5, 0≤c1≤0.3, 0≤c2≤0.3, 0≤c3≤0.3, 0≤c4≤0.3, 0≤c5≤0.3, 0≤d≤0.3, 0≤e≤0.3, and 0≤f≤0.3, provided that 0≤a1+a2≤1.0, a1+a2+a3+b1+b2+c1+c2+c3+c4+d+e+f≤1.0.

The polymer preferably has a weight average molecular weight (Mw) of 1000 to 500000, and more preferably 3000 to 100000. A Mw within the range provides satisfactory etching resistance and eliminates the risk of reduced resolution due to a failure to secure a difference in dissolution rate before and after exposure. As used herein, Mw refers to a polystyrene-equivalent measurement value obtained by gel permeation chromatography (GPC) using THF or N,N-dimethylformamide (DMF) as the solvent.

Furthermore, the finer the pattern rule, the stronger the influence of the molecular weight distribution (Mw/Mn) of the polymer. Therefore, the polymer preferably has a narrow dispersity Mw/Mn of 1.0 to 2.0 in order to provide a resist composition suitable for micropatterning to a small feature size. Within the above range, few polymers exhibit either low or high molecular weights, thereby reducing the risk of foreign matter appearing on the pattern after exposure or any deterioration in the pattern shape.

To synthesize the polymer, for example, the monomer providing the repeating unit is polymerized by adding a radical polymerization initiator in an organic solvent, followed by heating.

Specific examples of the organic solvent used for polymerization include toluene, benzene, THF, diethyl ether, dioxane, cyclohexane, cyclopentane, methyl ethyl ketone (MEK), propylene glycol monomethyl ether acetate (PGMEA), and γ-butyrolactone (GBL). Specific examples of the polymerization initiator include 2,2′-azobisisobutyronitrile (AIBN), 2,2′-azobis(2,4-dimethylvaleronitrile), dimethyl-2,2-azobis(2-methylpropionate), 1,1′-azobis(1-acetoxy-1-phenylethane) benzoyl peroxide, and lauroyl peroxide. The amount of the initiator added is preferably 0.01 to 25 mol % based on the total of monomers to be polymerized. The reaction temperature is preferably 50 to 150° C., and more preferably 60 to 100° C. The reaction time is preferably 2 to 24 hours, and from the viewpoint of production efficiency, more preferably 2 to 12 hours.

The polymerization initiator may be added to a monomer solution and supplied to a reaction vessel, or an initiator solution may be prepared separately from the monomer solution, with each solution being supplied to the reaction vessel independently. Since radicals generated from the initiator during the standby period are likely to promote a polymerization reaction to form an ultra-high molecular weight polymer, however, the monomer solution and the initiator solution are preferably each independently prepared and added dropwise, from the viewpoint of quality control. The acid labile group that has been incorporated in the monomer may be kept as such or polymerized, followed by protection or partial protection. Any known chain transfer agent, such as dodecyl mercaptan or 2-mercaptoethanol, may be used in combination for a molecular weight control purpose. In this case, the amount of the chain transfer agent added is preferably 0.01 to 20 mol % based on the total of monomers to be polymerized.

In the case of a monomer containing a hydroxy group, the hydroxy group may be substituted during polymerization with an acetal group, such as an ethoxyethoxy group, which can be readily deprotected with an acid. After polymerization, deprotection is carried out using a weak acid and water. Alternatively, the hydroxyl group may be substituted with a group such as an acetyl group, a formyl group, or a pivaloyl group, and subjected to alkaline hydrolysis after polymerization.

In the case of copolymerizing hydroxystyrene or hydroxyvinyl naphthalene, the hydroxystyrene or hydroxyvinyl naphthalene and other monomers may be dissolved in an organic solvent, followed by the addition of a radical polymerization initiator, and heated for polymerization. Alternatively, acetoxystyrene or acetoxyvinyl naphthalene is used instead. The acetoxy group may be deprotected by alkaline hydrolysis after polymerization to yield polyhydroxystyrene or hydroxy polyvinyl naphthalene.

For alkaline hydrolysis, a base such as aqueous ammonia or triethylamine may be used. The reaction temperature is preferably −20 to 100° C., and more preferably 0 to 60° C. The reaction time is preferably 0.2 to 100 hours, and more preferably 0.5 to 20 hours.

The amounts of monomers in the monomer solution may be appropriately determined, for example, to achieve the preferred content ratio of the repeating units described above.

The polymer obtained by the above production method may be used as the final product in various forms. The reaction solution resulting from the polymerization reaction may be used as the final product. Alternatively, the polymer may be recovered in powder form through a purifying step, such as a re-precipitation step of adding the polymerization solution to a poor solvent, and the resulting powder is used as the final product. From the viewpoints of operation efficiency and consistent quality, however, it is preferred to handle a polymer solution obtained by dissolving the powder resulting from the purifying step in a solvent as the final product.

Specific examples of the solvent to be used herein include, as described in paragraphs [0144] to [0145] of JP 2008-111103 A, ketones such as cyclohexanone and methyl-2-n-pentyl ketone; alcohols such as 3-methoxybutanol, 3-methyl-3-methoxybutanol, 1-methoxy-2-propanol, 1-ethoxy-2-propanol; ethers such as propylene glycol monomethyl ether (PGME), ethylene glycol monomethyl ether, propylene glycol monoethyl ether, ethylene glycol monoethyl ether, propylene glycol dimethyl ether, and diethylene glycol dimethyl ether; esters such as PGMEA, propylene glycol monoethyl ether acetate, ethyl lactate, ethyl pyruvate, butyl acetate, methyl 3-methoxypropionate, ethyl 3-ethoxypropionate, tert-butyl acetate, tert-butyl propionate, propylene glycol mono-tert-butyl ether acetate; lactones such as GBL; alcohols such as diacetone alcohol (DAA); high-boiling alcohols such as diethylene glycol, propylene glycol, glycerol, 1,4-butanediol, and 1,3-butanediol; and mixed solvents thereof.

In the polymer solution, the polymer concentration is preferably 0.01 to 30% by mass, and more preferably 0.1 to 20% by mass.

The reaction solution or polymer solution is preferably filtered through a filter. Filtration is effective for consistent quality because foreign matter and gel which may cause defects are removed.

Examples of materials for the filter used in the filtration include fluorocarbon-based, cellulose-based, nylon-based, polyester-based, and hydrocarbon-based materials. In the filtration step for the resist composition, however, a filter made of fluorocarbons commonly known as Teflon (registered trademark), hydrocarbons such as polyethylene and polypropylene, or nylon is preferred. While the pore size of the filter may be selected appropriately to comply with the desired cleanness, the filter preferably has a pore size of 100 nm or less, more preferably 20 nm or less. A single filter may be used, or a plurality of filters may be used in combination. Although the filtration method may simply involve passing the solution through once, the filtration is preferably carried out several times by flowing the solution in a circulating manner. In the polymer production process, the filtering step may be carried out any times in any order. However, filtering the reaction solution after polymerization reaction, the polymer solution, or both is preferred.

The base polymer (B) may be used singly or in combination with two or more polymers having different composition ratios, Mw, and/or Mw/Mn. The base polymer (B) may also include, in addition to the polymer, a hydrogenation product of a ring-opening metathesis polymer, and the hydrogenation product described in JP 2003-66612 A may be used.

The chemically amplified resist composition may further contain at least one selected from the group consisting of an organic solvent, a quencher, a photoacid generator other than the photoacid generator, and a surfactant. Hereinafter, each component will be described.

### (C) Organic Solvent

The chemically amplified resist composition of the present invention may contain an organic solvent as a component (C). The organic solvent (C) is not particularly limited as long as the aforementioned and other components are soluble therein. Specific examples of such an organic solvent include ketones such as cyclopentanone, cyclohexanone, and methyl-2-n-pentyl ketone; alcohols such as 3-methoxybutanol, 3-methyl-3-methoxybutanol, 1-methoxy-2-propanol, and 1-ethoxy-2-propanol; keto-alcohols such as DAA; ethers such as PGME, ethylene glycol monomethyl ether, propylene glycol monoethyl ether, ethylene glycol monoethyl ether, propylene glycol dimethyl ether, and diethylene glycol dimethyl ether; esters such as PGMEA, propylene glycol monoethyl ether acetate, ethyl lactate, ethyl pyruvate, butyl acetate, methyl 3-methoxypropionate, ethyl 3-ethoxypropionate, tert-butyl acetate, tert-butyl propionate, and propylene glycol mono-tert-butyl ether acetate; lactones such as GBL; and mixed solvents thereof.

Of these organic solvents, 1-ethoxy-2-propanol, PGMEA, cyclohexanone, GBL, DAA, and mixed solvents thereof are preferred because the base polymer of the component (B) exhibits particularly excellent solubility.

In the chemically amplified resist composition of the present invention, the content of the organic solvent (C) is preferably 200 to 5000 parts by mass, and more preferably 400 to 3500 parts by mass, based on 80 parts by mass of the base polymer (B). The organic solvents (C) may be used singly or in admixture of two or more thereof.

### (D) Quencher

The chemically amplified resist composition of the present invention may contain a quencher as a component (D). As used herein, the quencher refers to a material capable of trapping the acid generated from the photoacid generator in the chemically amplified resist composition to prevent the acid from diffusing into an unexposed region and forming the desired pattern.

The quencher (D) includes an onium salt represented by formula (2) or (3).

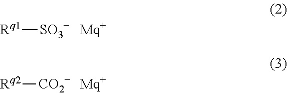

In formula (2), R^{q1} is a hydrogen atom or a hydrocarbyl group having 1 to 40 carbon atoms and optionally comprising a heteroatom, excluding the hydrocarbyl group in which the hydrogen atom bonded to the carbon atom at the α-position of the sulfo group is substituted with a fluorine atom or a fluoroalkyl group. In formula (3), R^{q2} is a hydrogen atom or a hydrocarbyl group having 1 to 40 carbon atoms and optionally comprising a heteroatom.

Specific examples of the hydrocarbyl group having 1 to 40 carbon atoms, represented by R^{q1}, include alkyl groups having 1 to 40 carbon atoms, such as a methyl group, an ethyl group, an n-propyl group, an isopropyl group, an n-butyl group, an isobutyl group, a sec-butyl group, a tert-butyl group, an n-pentyl group, a tert-pentyl group, an n-hexyl group, an n-octyl group, a 2-ethylhexyl group, an n-nonyl group, and an n-decyl group; cyclic saturated hydrocarbyl group having 3 to 40 carbon atoms, such as a cyclopentyl group, a cyclohexyl group, a cyclopentylmethyl group, a cyclopentylethyl group, a cyclopentylbutyl group, a cyclohexylmethyl group, a cyclohexylethyl group, a cyclohexylbutyl group, a norbornyl group, a tricyclo[5.2.1.0^{2,6}]decyl group, and an adamantyl group; and aryl groups having 6 to 40 carbon atoms, such as a phenyl group, a naphthyl group, and an anthracenyl group. Some or all of the hydrogen atoms in the hydrocarbyl group are optionally substituted with a group containing a heteroatom such as an oxygen atom, a sulfur atom, a nitrogen atom, or a halogen atom, and some of —CH₂— in the hydrocarbyl group are optionally substituted with a group containing a heteroatom such as an oxygen atom, a sulfur atom, or a nitrogen atom. As a result, the hydrocarbyl group may include, for instance, a hydroxy group, a fluorine atom, a chlorine atom, a bromine atom, an iodine atom, a cyano group, a carbonyl group, an ether bond, an ester bond, a sulfonate ester bond, a carbonate bond, a lactone ring, a sultone ring, a carboxylic anhydride(—C(═O)—O—C(═O)—), and a haloalkyl group.

Specific examples of the hydrocarbyl group represented by R^{q2} include, in addition to the substituents illustrated as specific examples of R^{q1}, fluorinated saturated hydrocarbyl groups, such as a trifluoromethyl group and a trifluoroethyl group, and fluorinated aryl groups such as a pentafluorophenyl group and a 4-trifluoromethylphenyl group.

Specific examples of the anion in the onium salt represented by formula (2) are given below, but not limited thereto.

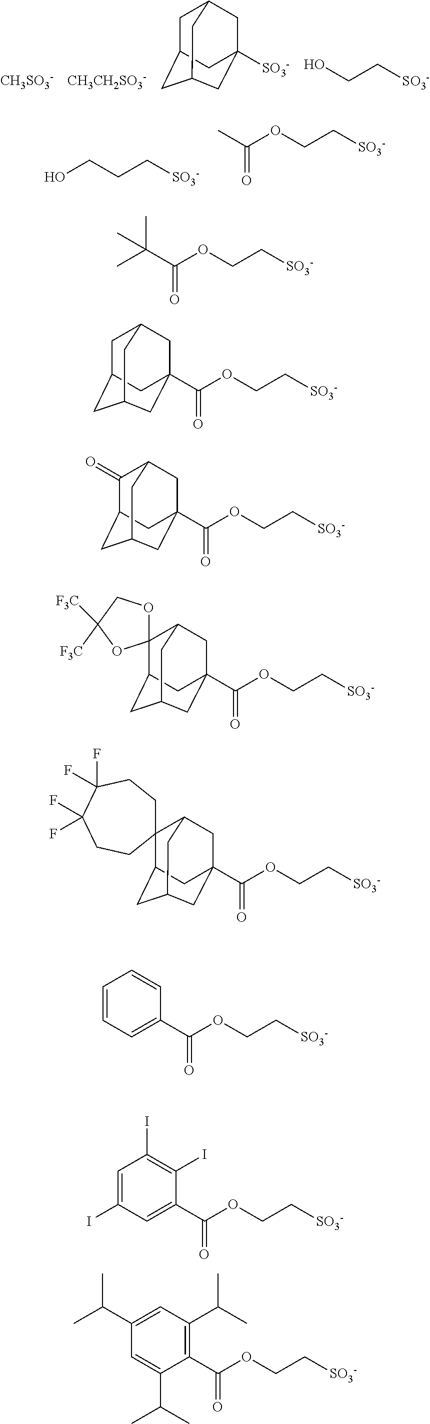

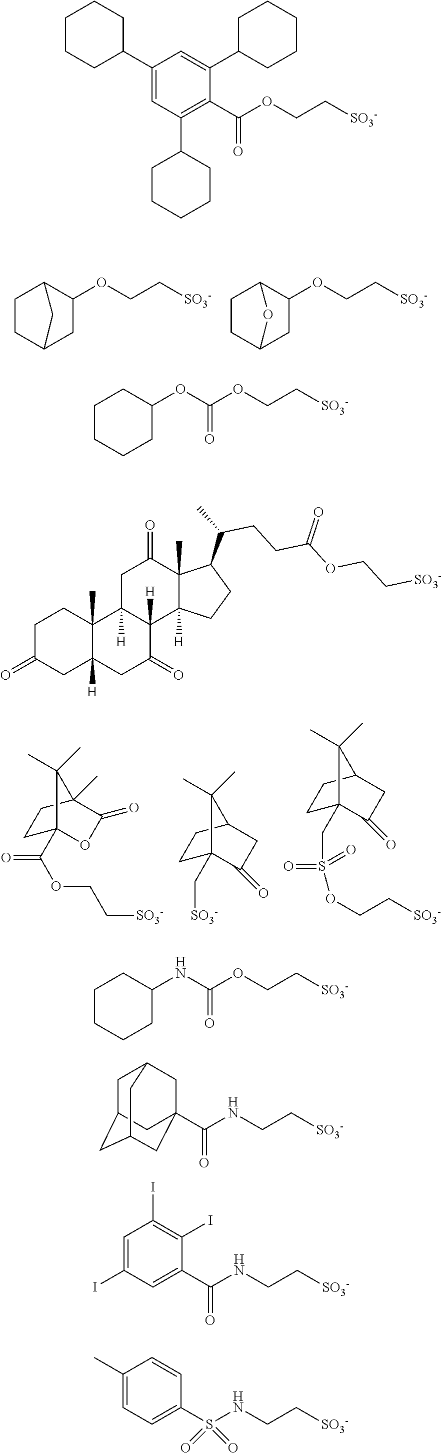

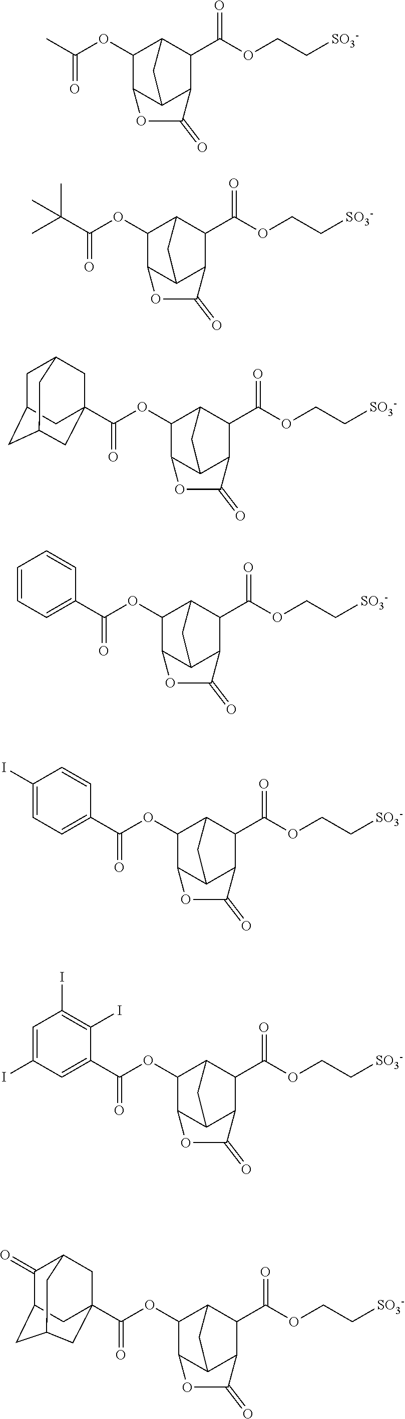

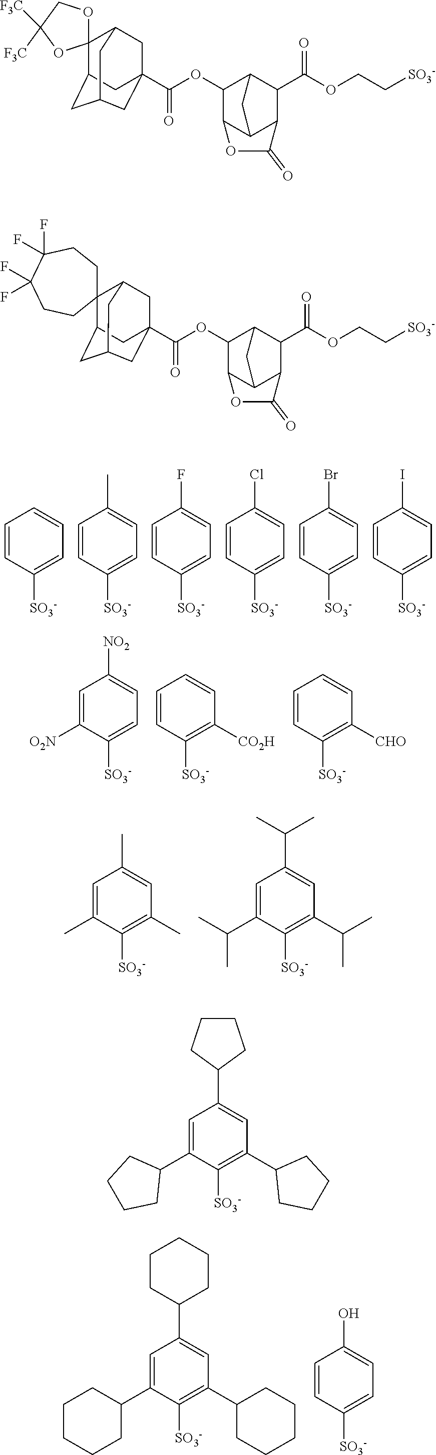

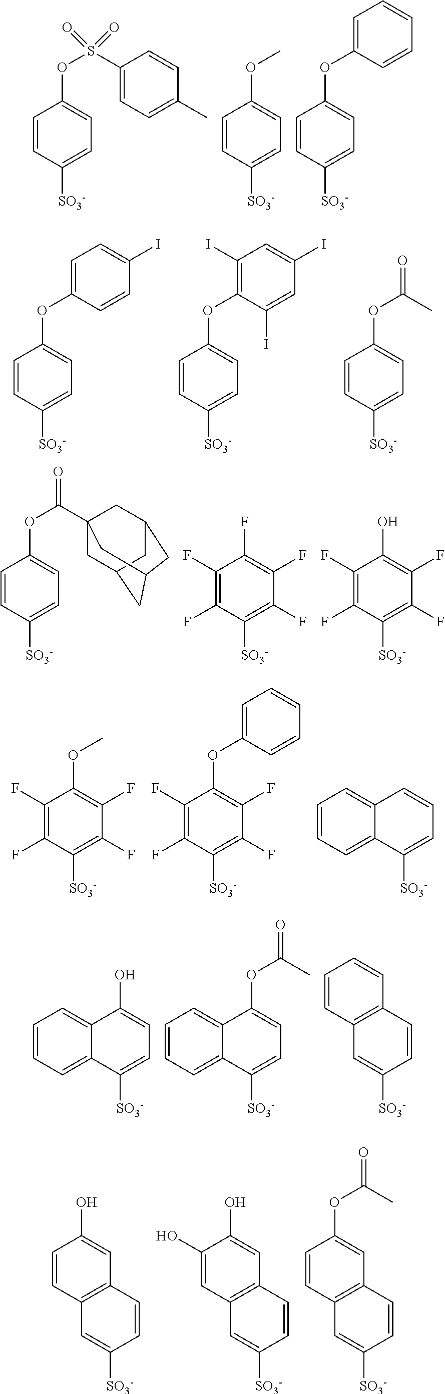

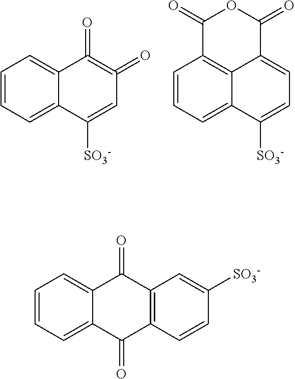

Specific examples of the anion in the onium salt represented by formula (3) are given below, but not limited thereto.

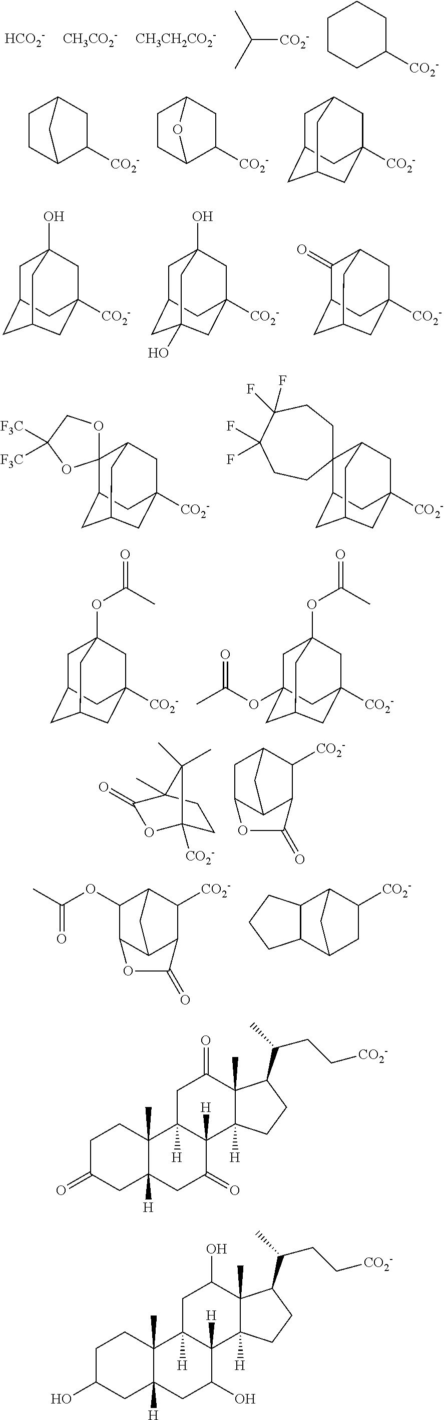

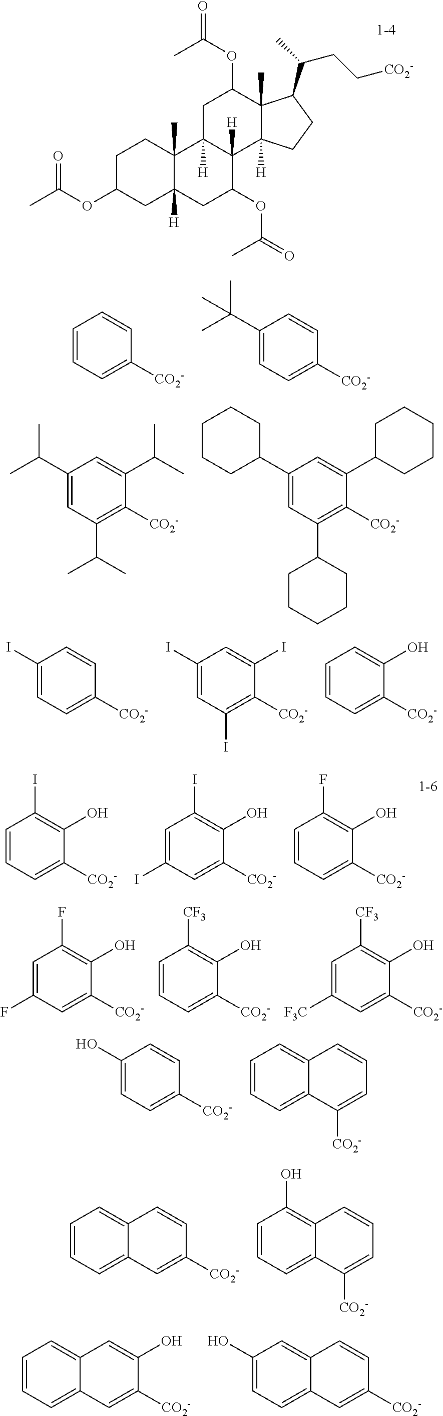

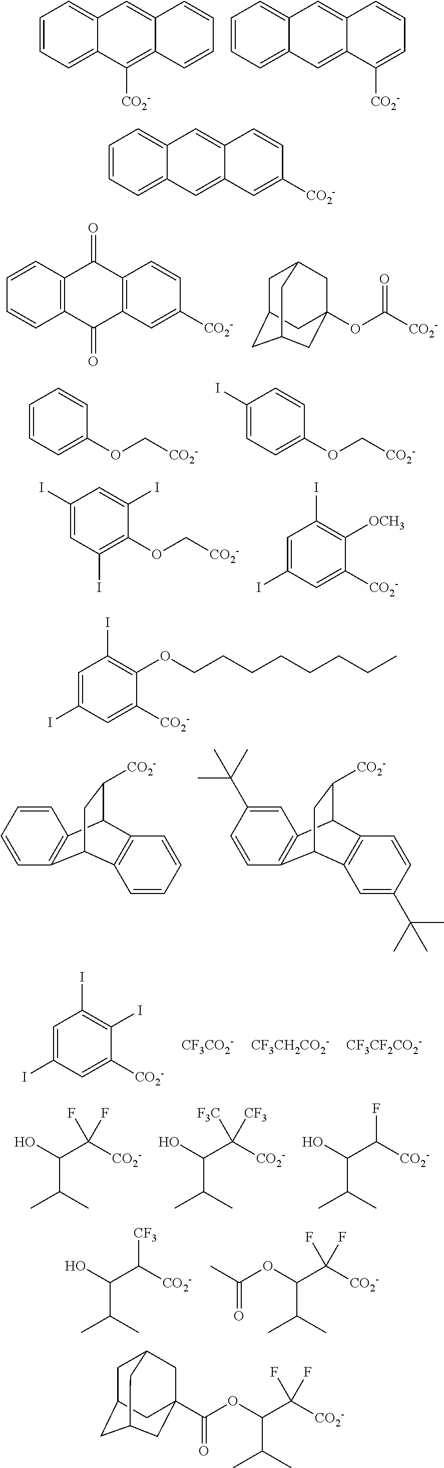

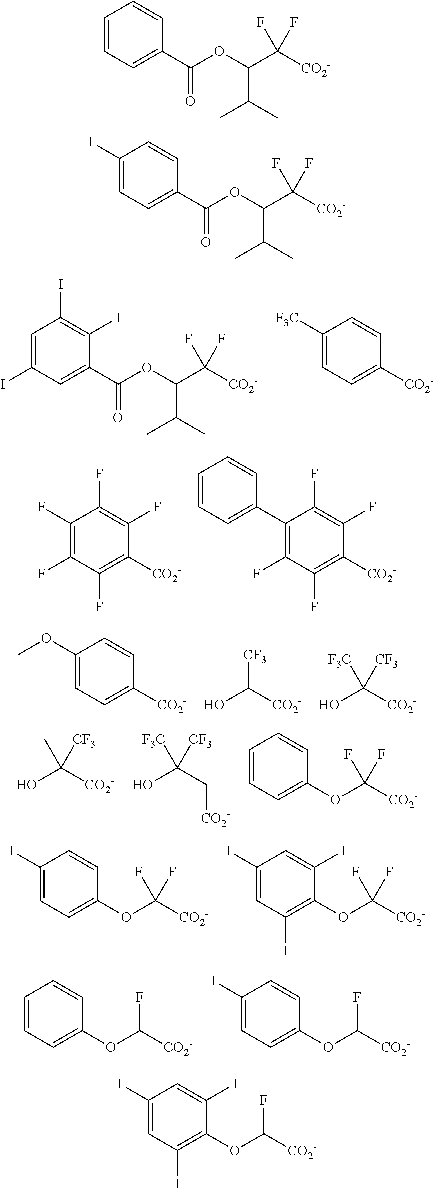

In formulae (2) and (3), Mq⁻ is an onium cation. Examples of the onium cation include a sulfonium cation, an iodonium cation, and an ammonium cation. Specific examples of the sulfonium cation include, but are not limited to, cations illustrated as specific examples of the sulfonium cation in general formula (1B), described in paragraphs [0102] to [0125] of JP 2024-3744 A, paragraphs [0044] to [0049] of WO 2024/128017 A1, and paragraphs [0035] to [0046] of JP 7491173 B, and illustrated as specific examples of the sulfonium cation in formula (d3). Specific examples of the iodonium cation include, but are not limited to, cations described in the paragraph [0181] of JP 2024-259 A. Specific examples of the ammonium cation include cations represented by formula (am-1).

Specific examples of the onium salt represented by formula (2) or (3) include any combination of the anions and cations described above. These onium salts may be easily prepared using known organic chemical methods by ion exchange reaction. For the ion exchange reaction, reference may be made to JP 2007-145797 A, for example.

The onium salt represented by formula (2) or (3) functions as a quencher in the chemically amplified resist composition of the present invention because the counter anion of the onium salt is a conjugated base of a weak acid. The weak acid used herein indicates an acidity insufficient to deprotect an acid labile group from an acid labile group-containing unit used in the base polymer. The onium salt represented by formula (2) or (3) functions as a quencher when used in combination with an onium salt-type photoacid generator having a conjugated base of a strong acid, such as a-fluorinated sulfonic acid, as the counter anion. In a system using a mixture of an onium salt that generates a strong acid, such as a-fluorinated sulfonic acid, and an onium salt that generates a weak acid, such as non-fluorinated sulfonic acid or carboxylic acid, if the strong acid generated from the photoacid generator upon exposure to high-energy radiation collides with the unreacted onium salt having a weak acid anion, then a salt exchange occurs whereby the weak acid is released and an onium salt having a strong acid anion is formed. In this course, the strong acid is exchanged into the weak acid having a low catalysis, incurring apparent deactivation of the acid for enabling acid diffusion control.

As the quencher (D), onium salts having a sulfonium cation and a phenoxide anion site in a common molecule as described in JP 6848776 B, a sulfonium cation and a carboxylate anion site in a common molecule as described in JP 6583136 B and JP 2020-200311 A, and an iodonium cation and a carboxylate anion site in a common molecule as described in JP 6274755 B may also be used.

When a photoacid generator that generates a strong acid is an onium salt, an exchange from the strong acid generated upon exposure to high-energy radiation to a weak acid as above can take place, but the weak acid generated upon exposure to high-energy radiation is considered to hardly collide with the unreacted onium salt that generates a strong acid to induce a salt exchange. This phenomenon is attributed to the fact that an onium cation tends to form an ion pair with a stronger acid anion.

When the chemically amplified resist composition of the present invention contains the onium salt represented by formula (2) or (3) as the quencher (D), the content is preferably 0.1 to 20 parts by mass, and more preferably 0.1 to 10 parts by mass, based on 80 parts by mass of the base polymer (B). Within the above range, the onium salt-type quencher of the component (D) is preferable since a satisfactory resolution is available without a substantial lowering of sensitivity. The onium salt represented by formula (2) or (3) may be used singly or in combination with two or more thereof.

The chemically amplified resist composition of the present invention may contain a nitrogen-containing compound as a quencher (D). Examples of the nitrogen-containing compound as the component (D) include primary, secondary, and tertiary amine compounds described in paragraphs [0146] to [0164] of JP 2008-111103 A, specifically amine compounds having a hydroxy group, an ether bond, an ester bond, a lactone ring, a cyano group, or a sulfonate ester bond. Primary and secondary amine compounds protected with a carbamate group are also included, as described in JP 3790649 B.

A sulfonium salt of sulfonic acid having a nitrogen-containing substituent may also be used as the nitrogen-containing compound. Such a compound functions as a quencher in the unexposed region, but as a so-called photo-degradable base in the exposed region because it loses the quencher function in the exposed region due to neutralization thereof with the acid generated by itself. Using a photo-degradable base, the contrast between exposed and unexposed regions can be further enhanced. For the photo-degradable base, reference may be made to, for example, JP 2009-109595 A and JP 2012-46501 A.

When the chemically amplified resist composition of the present invention contains the nitrogen-containing compound as the quencher (D), the content is preferably 0.001 to 12 parts by mass, and more preferably 0.01 to 8 parts by mass, based on 80 parts by mass of the base polymer (B). The nitrogen-containing compound may be used singly or in combination with two or more thereof.

### (E) Other Photoacid Generator

The chemically amplified resist composition of the present invention may contain, as a component (E), a photoacid generator other than the component (A) (hereinafter also referred to as another photoacid generator). The other photoacid generator is not particularly limited as long as the photoacid generator is a compound that generates acid upon high-energy radiation. The suitable other photoacid generator is represented by formula (4) or (5).

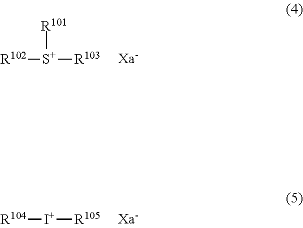

In formula (4), R¹⁰¹ to R¹⁰⁵ are each independently a hydrocarbyl group having 1 to 20 carbon atoms and optionally comprising a heteroatom. In addition, any two of R¹⁰¹, R¹⁰², and R¹⁰³ are optionally bonded to each other to form a ring together with a sulfur atom to which the two of R¹⁰¹, R¹⁰², and R¹⁰³ are bonded.

Examples of the cation of the sulfonium salt represented by formula (4) include, but are not limited to, cations illustrated as specific examples of the sulfonium cation in formula (1B), described in paragraphs [0102] to [0125] of JP 2024-3744 A, paragraphs [0044] to [0049] of WO 2024/128017 A1, and paragraphs [0035] to [0046] of JP 7491173 B, and illustrated as specific examples of the sulfonium cation in formula (d3). Specific examples of the cation of the iodonium salt represented by formula (5) include, but are not limited to, cations described in paragraph [0181] of JP 2024-259 A.

In formulae (4) and (5), Xa⁻ is an anion of a strong acid. The anion of the strong acid includes an anion represented by any one of formulae (c1-1) to (c1-5).

Another photoacid generator of the component (E) is preferably represented by formula (6).

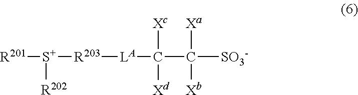

In formula (6), R²⁰¹ and R²⁰² are each independently a hydrocarbyl group having 1 to 30 carbon atoms and optionally comprising a heteroatom. R²⁰³ is a hydrocarbylene group having 1 to 30 carbon atoms and optionally comprising a heteroatom. In addition, any two of R²⁰¹, R²⁰², and R²⁰³ are optionally bonded to each other to form a ring together with a sulfur atom to which the two of R²⁰¹, R²⁰², and R²⁰³ are bonded.

The hydrocarbyl groups having 1 to 30 carbon atoms, represented by R²⁰¹ and R²⁰², may be either saturated or unsaturated and linear, branched, or cyclic. Specific examples thereof include alkyl groups having 1 to 30 carbon atoms, such as a methyl group, an ethyl group, an n-propyl group, an isopropyl group, an n-butyl group, an isobutyl group, a sec-butyl group, a tert-butyl group, a tert-pentyl group, an n-pentyl group, an n-hexyl group, an n-octyl group, a 2-ethylhexyl group, an n-nonyl group, and an n-decyl group; cyclic saturated hydrocarbyl group having 3 to 30 carbon atoms, such as a cyclopentyl group, a cyclohexyl group, a cyclopentylmethyl group, a cyclopentylethyl group, a cyclopentylbutyl group, a cyclohexylmethyl group, a cyclohexylethyl group, a cyclohexylbutyl group, a norbornyl group, an oxanorbornyl group, a tricyclo[5.2.1.0^{2,6}]decyl group, and an adamantyl group; aryl groups having 6 to 30 carbon atoms, such as a phenyl group, a methylphenyl group, an ethylphenyl group, an n-propylphenyl group, an isopropylphenyl group, an n-butylphenyl group, an isobutylphenyl group, a sec-butylphenyl group, a tert-butylphenyl group, a naphthyl group, a methylnaphthyl group, an ethylnaphthyl group, an n-propylnaphthyl group, an isopropylnaphthyl group, an n-butylnaphthyl group, an isobutylnaphthyl group, a sec-butylnaphthyl group, a tert-butylnaphthyl group, and an anthracenyl group; and group obtained by combining these groups. Some or all of the hydrogen atoms in the hydrocarbyl group are optionally substituted with a group containing a heteroatom such as an oxygen atom, a sulfur atom, a nitrogen atom, or a halogen atom, and some of —CH₂— in the hydrocarbyl group are optionally substituted with a group containing a heteroatom such as an oxygen atom, a sulfur atom, or a nitrogen atom. As a result, the hydrocarbyl group may include, for instance, a hydroxy group, a cyano group, a fluorine atom, a chlorine atom, a bromine atom, an iodine atom, a carbonyl group, an ether bond, an ester bond, a sulfonate ester bond, a carbonate bond, a lactone ring, a sultone ring, a carboxylic anhydride(—C(═O)—O—C(═O)—), and a haloalkyl group.

The hydrocarbylene group having 1 to 30 carbon atoms, represented by R²⁰³, may be either saturated or unsaturated and linear, branched, or cyclic. Specific examples thereof include alkanediyl groups having 1 to 30 carbon atoms, such as a methanediyl group, an ethane-1,1-diyl group, an ethane-1,2-diyl group, a propane-1,3-diyl group, a butane-1,4-diyl group, a pentane-1,5-diyl group, a hexane-1,6-diyl group, a heptane-1,7-diyl group, an octane-1,8-diyl group, a nonane-1,9-diyl group, a decane-1,10-diyl group, an undecane-1,11-diyl group, a dodecane-1,12-diyl group, a tridecane-1,13-diyl group, a tetradecane-1,14-diyl group, a pentadecane-1,15-diyl group, a hexadecane-1,16-diyl group, and a heptadecane-1,17-diyl group; a cyclic saturated hydrocarbylene groups having 3 to 30 carbon atoms, such as a cyclopentanediyl group, a cyclohexanediyl group, a norbornanediyl group, and an adamantanediyl group; and arylene groups such as a phenylene group, a methylphenylene group, an ethylphenylene group, an n-propylphenylene group, an isopropylphenylene group, an n-butylphenylene group, an isobutylphenylene group, a sec-butylphenylene group, a tert-butylphenylene group, a naphthylene group, a methylnaphthylene group, an ethylnaphthylene group, an n-propylnaphthylene group, an isopropylnaphthylene group, an n-butylnaphthylene group, an isobutylnaphthylene group, a sec-butylnaphthylene group, and a tert-butylnaphthylene group. Some or all of the hydrogen atoms in the hydrocarbylene group are optionally substituted with a group containing a heteroatom such as an oxygen atom, a sulfur atom, a nitrogen atom, or a halogen atom, and some of —CH₂— in the hydrocarbylene group are optionally substituted with a group containing a heteroatom such as an oxygen atom, a sulfur atom, or a nitrogen atom. As a result, the hydrocarbyl group may include, for instance, a hydroxy group, a cyano group, a fluorine atom, a chlorine atom, a bromine atom, an iodine atom, a carbonyl group, an ether bond, an ester bond, a sulfonate ester bond, a carbonate bond, a lactone ring, a sultone ring, a carboxylic anhydride(—C(═O)—O—C(═O)—), and a haloalkyl group. The heteroatom is preferably an oxygen atom.

In formula (6), L^{A} is a single bond, an ether bond, or a hydrocarbylene group having 1 to 20 carbon atoms and optionally comprising a heteroatom. The hydrocarbylene group may be either saturated or unsaturated and linear, branched, or cyclic. Specific examples thereof include the same hydrocarbylene group represented by R²⁰³ as illustrated.

In formula (6), X^{a}, X^{b}, X^{c}, and X^{d} are each independently a hydrogen atom, a fluorine atom, or a trifluoromethyl group, provided that at least one of the X^{a}, X^{b}, X^{c}, and X^{d} is a fluorine atom or a trifluoromethyl group.

The photoacid generator represented by formula (6) is preferably represented by formula (6′).

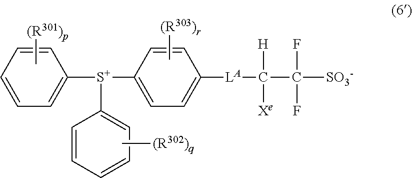

In formula (₆′), LA is the same as above. Xe is a hydrogen atom or a trifluoromethyl group, preferably a trifluoromethyl group. R³⁰¹, R³⁰², and R³⁰³ are each independently a hydrogen atom or a hydrocarbyl group having 1 to 20 carbon atoms and optionally comprising a heteroatom. The hydrocarbyl group may be either saturated or unsaturated and may be linear, branched, or cyclic. Specific examples thereof include the same hydrocarbyl group represented by R^{fa1} in formula (c1-1-1) as illustrated. “p” and “q” are each independently an integer of 0 to 5, and “r” is an integer of 0 to 4.

Examples of the photoacid generator represented by formula (6) include the same photoacid generator represented by formula (2) in JP 2017-26980 A, as illustrated.

Of the other photoacid generators, photoacid generators containing the anion represented by formula (c1-1-1) or (c1-4) are particularly preferable because of reduced acid diffusion and high solubility in the solvent. In addition, the photoacid generator represented by formula (6′) is particularly preferable because of extremely reduced acid diffusion.

When the chemically amplified resist composition of the present invention contains the other photoacid generator (E), the content is preferably 0.1 to 40 parts by mass, and more preferably 0.5 to 20 parts by mass, based on 80 parts by mass of the base polymer (B). An amount of the photoacid generator of the component (E) added within the range is preferable because a satisfactory resolution is available without the risk of foreign matter being formed after development or during stripping of the resist film. The other photoacid generator (E) may be used singly or in combination with two or more thereof.

### (F) Surfactant

The chemically amplified resist composition of the present invention may further contain a surfactant as a component (F). The surfactant (F) is preferably either a surfactant that is insoluble or substantially insoluble in water but soluble in an alkali developing solution, or a surfactant that is insoluble or substantially insoluble in water and an alkali developing solution. Such surfactants may be referenced in JP 2010-215608 A and JP 2011-16746 A.

Among the surfactants described in the aforementioned publications, the surfactant that is insoluble or substantially insoluble in water and alkali developing solution is preferably FC-4430 (manufactured by 3M Company), SURFLON (registered trademark) 5-381 (manufactured by AGC Seimi Chemical Co., Ltd.), Olfine (registered trademark) E1004 (manufactured by Nisshin Chemical Industries, Ltd.), KH-20 and KH-30 (manufactured by AGC Seimi Chemical Co., Ltd.), or an oxetane ring-opening polymer represented by formula (surf-1).

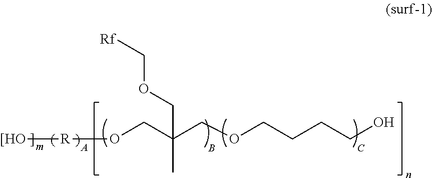

Herein, R, Rf, A, B, C, m, n are applied only to formula (surf-1), regardless of their descriptions. R is a di- to tetra-valent aliphatic group having 2 to 5 carbon atoms. Examples of the divalent aliphatic group include an ethylene group, a 1,4-butylene group, a 1,2-propylene group, a 2,2-dimethyl-1,3-propylene group, and a 1,5-pentylene group, and examples of the tri- or tetra-valent aliphatic group are given below:

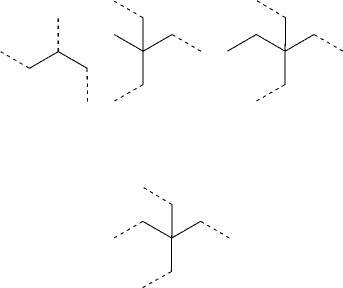

wherein the dashed line represents a bond, and each partial structure is derived from glycerol, trimethylolethane, trimethylolpropane, or pentaerythritol.

Of these, for instance, a 1,4-butylene group and a 2,2-dimethyl-1,3-propylene group are preferable.

Rf is a trifluoromethyl group or a pentafluoroethyl group, preferably a trifluoromethyl group. “m” is an integer of 0 to 3, “n” is an integer of 1 to 4, and the sum of “n” and “m”, which represents the valence of R, is an integer of 2 to 4. “A” is 1. “B” is an integer of 2 to 25, preferably an integer of 4 to 20. “C” is an integer of 0 to 10, preferably 0 or 1. The respective structural units in formula (surf-1) do not define the arrangement thereof and may be bonded in a block or randomly. For the production of partially fluorinated oxetane ring-opening polymer-based surfactants, reference may be made to U.S. Pat. No. 5,650,483 A, for example.

The surfactant that is insoluble or substantially insoluble in water but soluble in an alkali developing solution has the function of reducing the penetration of water and leaching by orientation on the surface of the resist film when a resist protective film is not used in ArF immersion lithography and is therefore effective in suppressing elution of water-soluble components from the resist film to reduce damage to the exposure apparatus. The surfactant is also effective since it becomes solubilized during aqueous alkaline development following exposure or post-exposure baking (PEB), thereby minimizing the formation of foreign matter that could lead to defects. Such a surfactant is preferably a polymeric surfactant that possesses the property of being insoluble or substantially insoluble in water but soluble in an alkali developing solution, also referred to as a hydrophobic resin, especially with high water-repellent properties and enhanced water slippage.

Specific examples of such a polymeric surfactant include those containing at least one selected from the group consisting of repeating units represented by formulae (7A) to (7E).

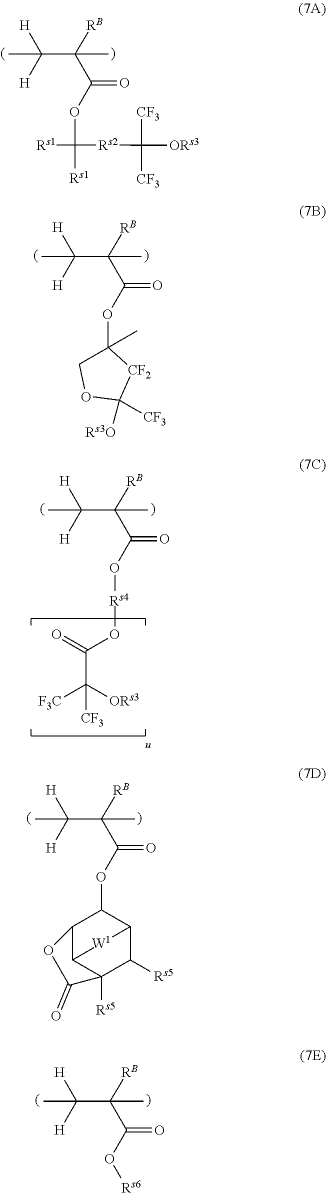

In formulae (7A) to (7E), RB is a hydrogen atom, a fluorine atom, a methyl group, or a trifluoromethyl group. W¹ is —CH₂—, —CH₂CH₂—, —O—, or two separate —H. Each R^{s1} is independently a hydrogen atom or a hydrocarbyl group having 1 to 10 carbon atoms. R^{s2} is a single bond or a linear or branched hydrocarbylene group having 1 to 5 carbon atoms. Each R^{s3} is independently a hydrogen atom, a hydrocarbyl group or fluorinated hydrocarbyl group having 1 to 15 carbon atoms, or an acid labile group. When R^{s3} is a hydrocarbyl group or a fluorinated hydrocarbyl group, an ether bond or a carbonyl group may intervene in a carbon-carbon bond. R^{s4} is a (u+1)-valent hydrocarbon group or fluorinated hydrocarbon group having 1 to 20 carbon atoms. “u” is an integer of 1 to 3. Each R^{s5} is independently a hydrogen atom or a group represented by —C(═O)—O—R^{sa}. R^{sa} is a fluorinated hydrocarbyl group having 1 to 20 carbon atoms. R^{s6} is a hydrocarbyl group or fluorinated hydrocarbyl group having 1 to 15 carbon atoms, wherein an ether bond or a carbonyl group may intervene in a carbon-carbon bond.

The hydrocarbyl groups having 1 to 10 carbon atoms, represented by R^{s1}, are preferably a saturated hydrocarbyl group and may be linear, branched, or cyclic. Specific examples thereof include alkyl groups having 1 to 10 carbon atoms, such as a methyl group, an ethyl group, an n-propyl group, an isopropyl group, an n-butyl group, an isobutyl group, a sec-butyl group, a tert-butyl group, an n-pentyl group, an n-hexyl group, an n-heptyl group, an n-octyl group, an n-nonyl group, and an n-decyl group; and cyclic saturated hydrocarbyl groups having 3 to 10 carbon atoms, such as a cyclopropyl group, a cyclobutyl group, a cyclopentyl group, a cyclohexyl group, an adamantyl group, and a norbornyl group. Of these, a hydrocarbyl group having 1 to 6 carbon atoms is preferable.

The hydrocarbylene group represented by R^{s2} is preferably a saturated hydrocarbylene group and may be linear, branched, or cyclic. Specific examples thereof include a methylene group, an ethylene group, a propylene group, a butylene group, and a pentylene group.

The hydrocarbyl group represented by R^{s3} or R^{s6} may be either saturated or unsaturated and linear, branched, or cyclic. Specific examples thereof include aliphatic unsaturated hydrocarbyl groups such as a saturated hydrocarbyl group, an alkenyl group, and an alkynyl group, and a saturated hydrocarbyl group is preferable. Examples of the saturated hydrocarbyl group include, in addition to the same hydrocarbyl group represented by R^{s1} as illustrated, an undecyl group, a dodecyl group, a tridecyl group, a tetradecyl group, and a pentadecyl group. Examples of the fluorinated hydrocarbyl group represented by R^{s3} or R^{s6} include groups in which some or all of the hydrogen atoms bonded to the carbon atoms of the aforementioned hydrocarbyl group are substituted with fluorine atoms. As described above, an ether bond or a carbonyl group may intervene in these carbon-carbon bonds.

Specific examples of the acid labile group represented by R^{s3} include the groups represented by formulae (AL-3) to (AL-5) described above, trialkylsilyl groups in which each alkyl moiety has 1 to 6 carbon atoms, and oxo-containing alkyl groups having 4 to 20 carbon atoms.

The (u+1)-valent hydrocarbon group or fluorinated hydrocarbon group represented by R^{s4} may be linear, branched, or cyclic, and specific examples thereof include groups obtained by removing the number u of hydrogen atoms from the aforementioned hydrocarbyl group or fluorinated hydrocarbyl group.

The fluorinated hydrocarbyl group represented by R^{sa} is preferably saturated and may be linear, branched, or cyclic. Specific examples thereof include the hydrocarbyl group in which some or all of the hydrogen atoms are substituted with fluorine atoms. Specific examples thereof include a trifluoromethyl group, a 2,2,2-trifluoroethyl group, a 3,3,3-trifluoro-1-propyl group, a 3,3,3-trifluoro-2-propyl group, a 2,2,3,3-tetrafluoropropyl group, a 1,1,1,3,3,3-hexafluoroisopropyl group, a 2,2,3,3,4,4,4-heptafluorobutyl group, a 2,2,3,3,4,4,5,5-octafluoropentyl group, a 2,2,3,3,4,4,5,5,6,6,7,7-dodecafluoroheptyl group, a 2-(perfluorobutyl)ethyl group, a 2-(perfluorohexyl)ethyl group, a 2-(perfluorooctyl)ethyl group, and a 2-(perfluorodecyl) ethyl group.

Specific examples of the repeating unit represented by any one of formulae (7A) to (7E) are given below, but not limited thereto. In formulae below, RB is the same as above.

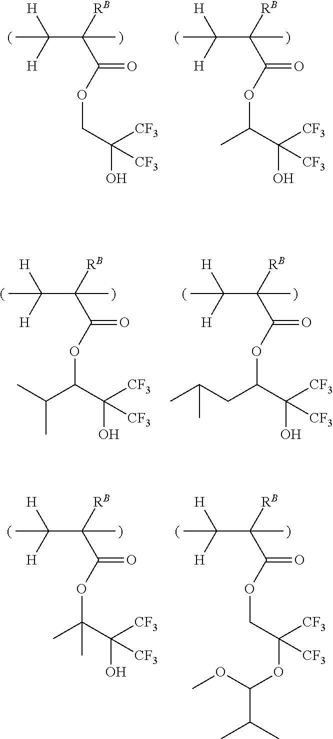

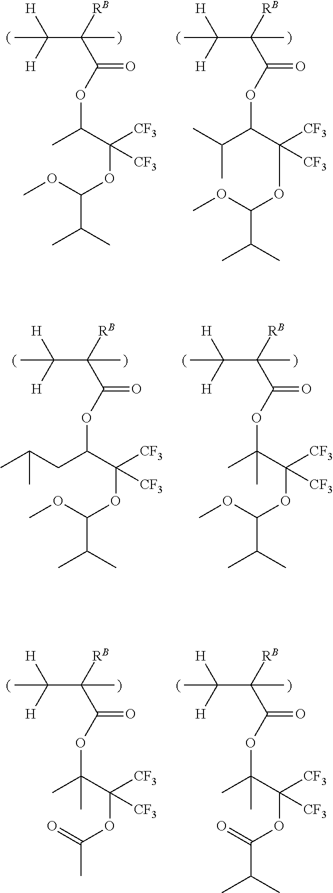

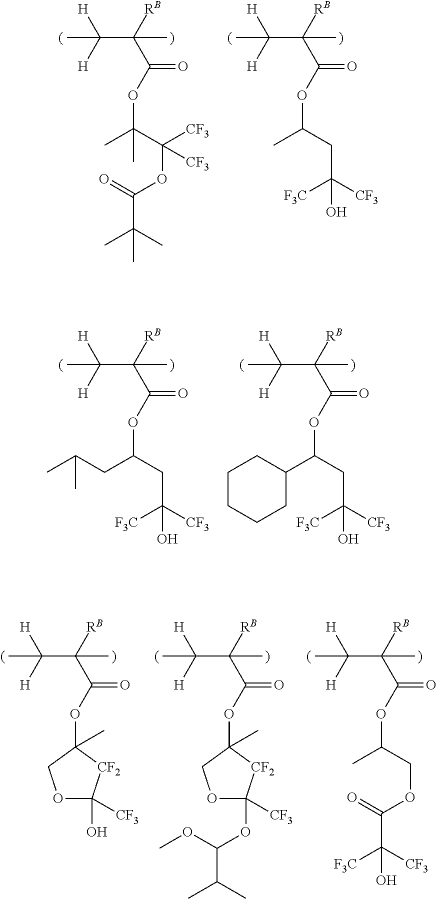

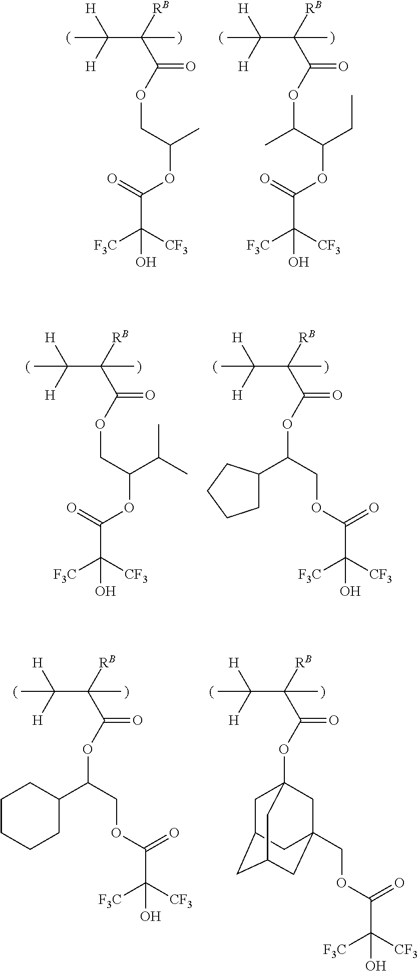

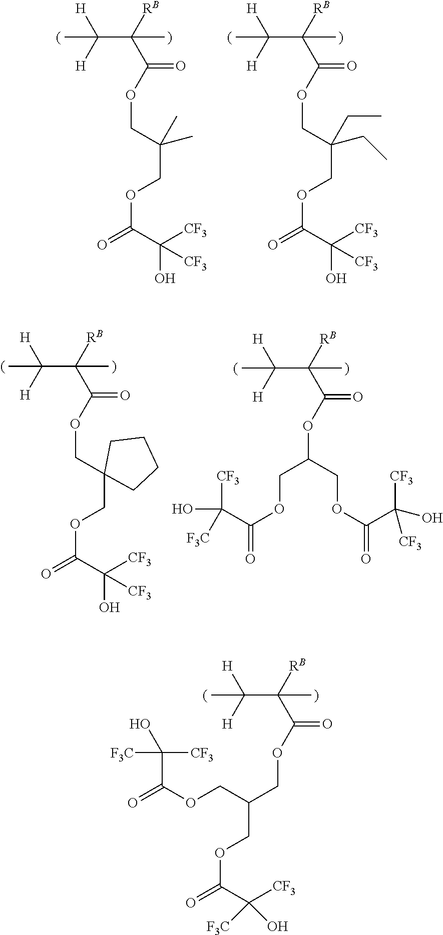

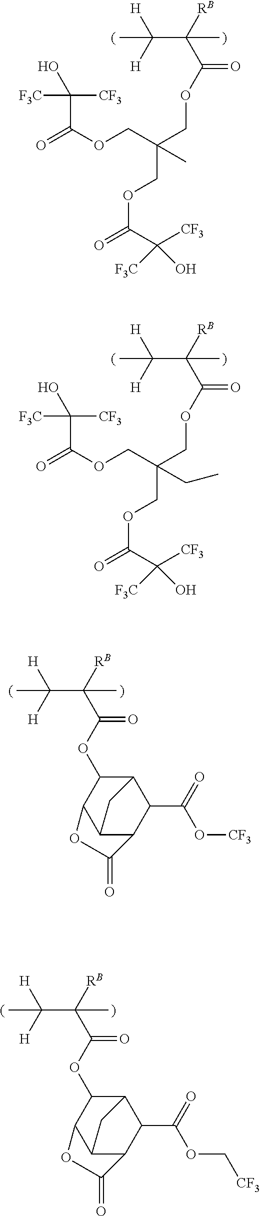

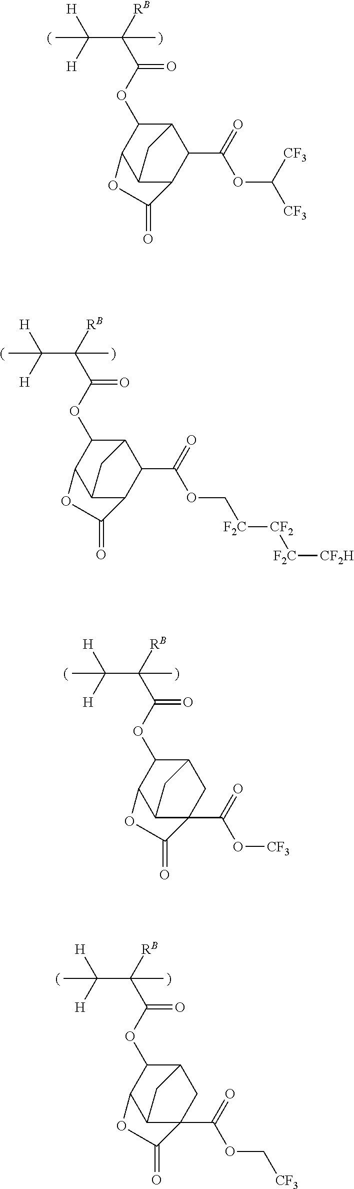

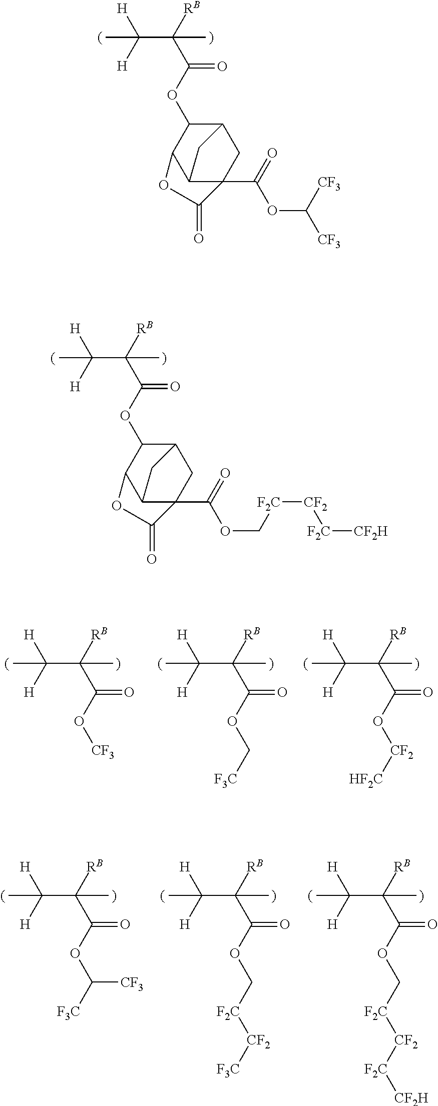

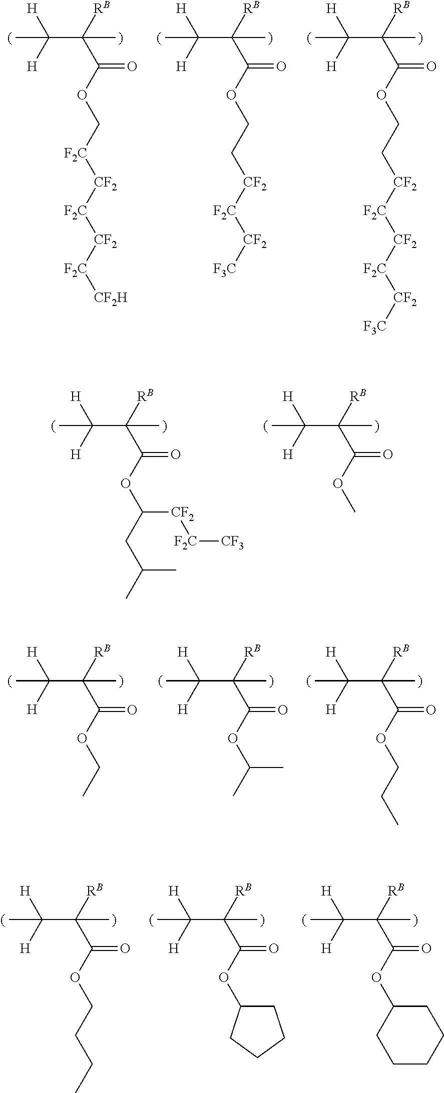

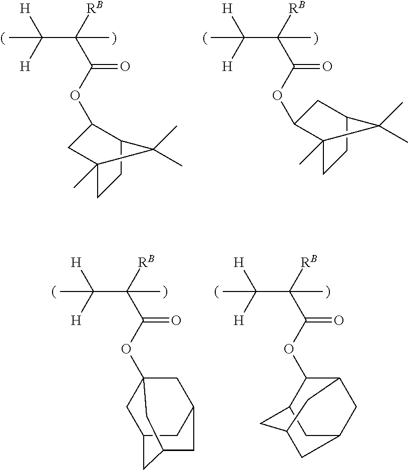

The polymeric surfactant may further contain a repeating unit other than the repeating units represented by formulae (7A) to (7E). Examples of the other repeating units include repeating units derived from methacrylic acid and α-trifluoromethylacrylic acid derivatives. In the polymeric surfactant, the content of the repeating units represented by formulae (7A) to (7E) is preferably 20 mol % or more, more preferably 60 mol % or more, further preferably 100 mol % based on the total repeating units.

The polymeric surfactant preferably has a Mw of 1000 to 500000, and more preferably 3000 to 100000. Mw/Mn is preferably 1.0 to 2.0, and more preferably 1.0 to 1.6.

The method of synthesizing the polymeric surfactant includes dissolving an unsaturated bond-containing monomer providing repeating units represented by formulae (7A) to (7E) and optionally other repeating units in an organic solvent, adding a radical initiator, and heating for polymerization. Examples of the organic solvent used for polymerization include toluene, benzene, THF, diethyl ether, and dioxane. Examples of the polymerization initiator include AIBN, 2,2′-azobis(2,4-dimethylvaleronitrile), dimethyl 2,2-azobis(2-methylpropionate), benzoyl peroxide, and lauroyl peroxide. The reaction temperature is preferably 50 to 100° C. The reaction time is preferably 4 to 24 hours. The acid labile group that has been incorporated in the monomer may be kept as such or polymerized, followed by protection or partial protection.

When the polymeric surfactant is synthesized, any known chain transfer agent such as dodecyl mercaptan or 2-mercaptoethanol may be used for a molecular weight control purpose. In this case, the amount of the chain transfer agent added is preferably 0.01 to 10 mol % based on the total number of moles of monomers to be polymerized.

When the chemically amplified resist composition of the present invention contains the surfactant (F), the content is preferably 0.1 to 50 parts by mass, and more preferably 0.5 to 10 parts by mass, based on 80 parts by mass of the base polymer (B). At least 0.1 parts by mass of the surfactant (F) is effective in improving the receding contact angle with water of the resist film at its surface. Up to 50 parts by mass of the surfactant (F) are effective in forming a resist film having a low rate of dissolution in a developing solution, thus maintaining the height of a fine pattern formed therein. The surfactant (F) may be used singly or in combination with two or more thereof.

### (G) Other Components

The chemically amplified resist composition of the present invention may contain other components (G), for example, a compound which is decomposed with an acid to generate another acid (i.e., acid amplifier compound), an organic acid derivative, a fluorine-substituted alcohol, and a compound having a Mw of 3000 or less whose solubility in the developing solution under the action of an acid (i.e., dissolution inhibitor). For the acid amplifier compound, reference may be made to the compounds described in JP 2009-269953 A or JP 2010-215608 A. When the acid amplifier compound is contained, the content is preferably 0 to 5 parts by mass, and more preferably 0 to 3 parts by mass, based on 80 parts by mass of the base polymer (B). An extra content of the acid amplifier compound can make the acid diffusion control difficult and cause degradations to resolution and pattern profile. For the organic acid derivative, fluorine-substituted alcohol, and dissolution inhibitor, reference may be made to JP 2009-269953 A or JP 2010-215608 A.

### Patterning Process

A patterning process of the present invention includes: forming a resist film on a substrate by using the chemically amplified resist composition; exposing the resist film to high-energy radiation; and developing the exposed resist film by using a developing solution.

The substrate used may be, for example, a substrate for integrated circuitry fabrication (e.g., Si, SiO₂, SiN, SiON, TiN, WSi, BPSG, SOG, an organic antireflective film) or a substrate for mask circuitry fabrication (e.g., Cr, CrO, CrON, MoSi₂, SiO₂)

A resist film is formed, for example, by applying the chemically amplified resist composition to a substrate using methods such as spin coating so that the film preferably has a thickness of 0.05 to 2 μm, followed by prebaking on a hot plate preferably at a temperature of 60 to 150° C. for 1 to 10 minutes, more preferably at 80 to 140° C. for 1 to 5 minutes.

Examples of high-energy radiation used for exposing a resist film include KrF excimer laser, ArF excimer laser, EB, and EUV having a wavelength of 3 to 15 nm. On use of KrF excimer laser, ArF excimer laser, or EUV, the resist film is exposed through a mask having the desired pattern in a dose of preferably 1 to 200 mJ/cm², more preferably 10 to 100 mJ/cm². On use of EB, a pattern may be written through a mask having the desired pattern or directly in a dose of preferably 1 to 300 μC/cm², more preferably 10 to 200 μC/cm².

The exposure may be performed by conventional lithography, whereas the immersion lithography of holding a liquid having a refractive index of 1.0 or more between the resist film and the projection lens. In this case, a protective film that is insoluble in water may be used.

The protective film that is insoluble in water serves to prevent any components from being leached out of the resist film and to improve water slippage on the film surface, and is generally divided into two types. The first type is an organic solvent-strippable protective film that must be stripped before aqueous alkaline development with an organic solvent in which the resist film is not soluble. The second type is an aqueous alkali-soluble protective film that is soluble in an alkali developing solution so that the protective film can be removed simultaneously with the removal of solubilized regions of the resist film. The latter is preferably of a material containing a polymer having a 1,1,1,3,3,3-hexafluoro-2-propanol residue, which is insoluble in water and soluble in an alkali developing solution, as a base in an alcohol solvent having 1 to 4 carbon atoms, an ether solvent having 8 to 12 carbon atoms, or a mixture thereof. Alternatively, the aforementioned surfactant that is insoluble in water and soluble in an alkali developing solution may be dissolved in an alcohol solvent having 1 to 4 carbon atoms, an ether solvent having 8 to 12 carbon atoms, or a mixed solvent thereof to form a material.

After the exposure, PEB may be performed by heating, for example, on a hot plate preferably at 60 to 150° C. for 1 to 5 minutes, more preferably at 80 to 140° C. for 1 to 3 minutes.

The resist film is developed with a developing solution, such as aqueous alkaline, preferably containing 0.1 to 5% by mass, more preferably 2 to 3% by mass of tetramethylammonium hydroxide (TMAH), for example. The development is preferably performed for 0.1 to 3 minutes, more preferably 0.5 to 2 minutes by conventional techniques such as dip, puddle, and spray techniques, resulting in dissolution of the exposed region, whereby a desired pattern is formed on the substrate.

After a resist film is formed, a step of rinsing with pure water may be introduced to extract, for instance, an acid generator from the film surface or wash away particles. After exposure, a step of rinsing may be introduced to remove any water remaining on the film.

A pattern may also be formed by a double patterning process. The double patterning process includes a trench process of processing an underlay to a 1:3 trench pattern by a first step of exposure and etching, shifting the position, and forming a 1:3 trench pattern by a second step of exposure for forming a 1:1 pattern; and a line process of processing a first underlay to a 1:3 isolated left pattern by a first step of exposure and etching, shifting the position, processing a second underlay formed below the first underlay by a second step of exposure through the 1:3 isolated left pattern, for forming a half-pitch 1:1 pattern.

In the patterning process of the present invention, a negative tone development technique may be used, in which an organic solvent is used as the developing solution instead of the aqueous alkaline to dissolve the unexposed region.

In the organic solvent development, the developing solution used may be, for example, 2-octanone, 2-nonanone, 2-heptanone, 3-heptanone, 4-heptanone, 2-hexanone, 3-hexanone, diisobutyl ketone, methylcyclohexanone, acetophenone, methylacetophenone, propyl acetate, butyl acetate, isobutyl acetate, pentyl acetate, butenyl acetate, isopentyl acetate, propyl formate, butyl formate, isobutyl formate, pentyl formate, isopentyl formate, methyl valerate, methyl pentenoate, methyl crotonate, ethyl crotonate, methyl propionate, ethyl propionate, ethyl 3-ethoxypropionate, methyl lactate, ethyl lactate, propyl lactate, butyl lactate, isobutyl lactate, pentyl lactate, isopentyl lactate, methyl 2-hydroxyisobutyrate, ethyl 2-hydroxyisobutyrate, methyl benzoate, ethyl benzoate, phenyl acetate, benzyl acetate, methyl phenylacetate, ethyl phenylacetate, benzyl formate, phenylethyl formate, methyl 3-phenylpropionate, benzyl propionate, or 2-phenylethyl acetate. These organic solvents may be used singly or in admixture of two or more thereof.

### Example

Hereinafter, Synthesis Examples, Examples, and Comparative Examples are given to describe the present invention in detail, but the present invention is not limited to these examples at all. Note that the apparatus used is as shown below.

MALDI TOF-MS: S3000 manufactured by JEOL Ltd.

### [1] Synthesis of Onium Salt

### [Example 1-1] Synthesis of Onium Salt PAG-1

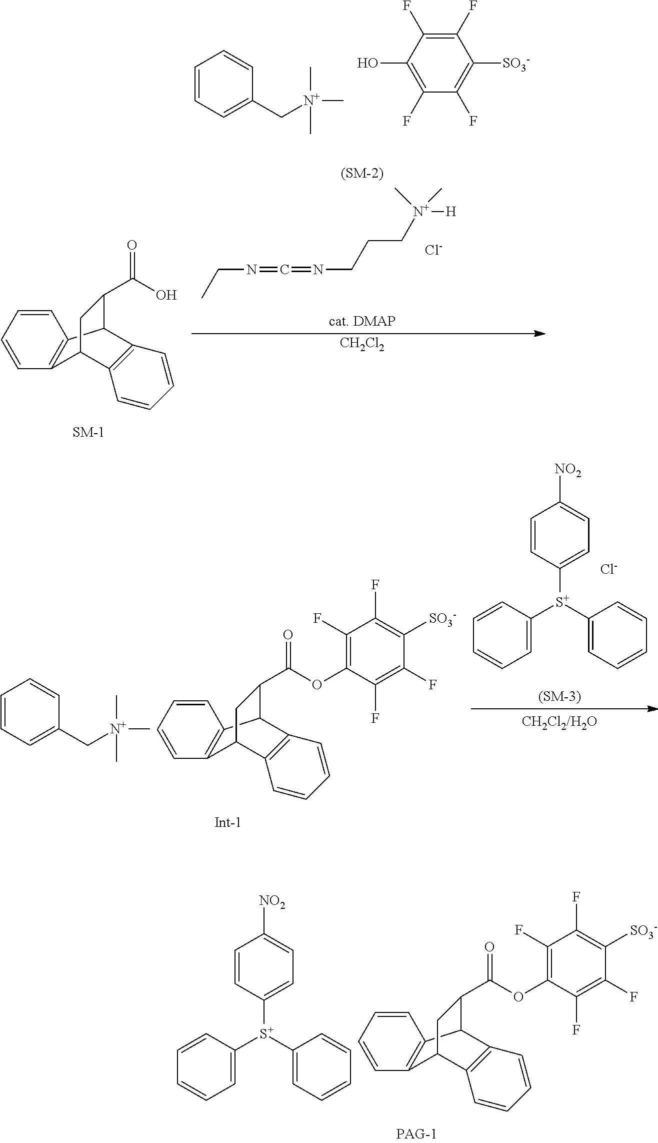

### (1) Synthesis of Intermediate In-1

In a nitrogen atmosphere, raw material SM-1 (6.3 g), raw material SM-2 (10.4 g), DMAP (4-dimethylaminopyridine) (0.3 g), and methylene chloride (50 g) were added to a reactor and cooled in an ice bath. While the temperature in the reactor was kept at 20° C. or lower, 1-ethyl-3-(3-dimethylaminopropyl)carbodiimide hydrochloride (5.8 g) was added as a powder. After the addition, the mixture was warmed up to room temperature and aged for 12 hours. After aging, water was added to stop the reaction, ordinary aqueous work-up was performed to distill off the solvent, followed by the addition of diisopropyl ether. The residue was washed to yield 20.4 g (yield: 92%) of intermediate In-1 as oily matter.

### (2) Synthesis of PAG-1

In a nitrogen atmosphere, the intermediate In-1 (14.4 g), an aqueous solution of raw material SM-3 (30.5 g, equivalent to 25.1 mol), and methylene chloride (50 g) were added, and the mixture was stirred for 15 minutes. Thereafter, the organic layer was separated, washed with water, and then concentrated under reduced pressure. Methyl isobutyl ketone (50 g) was added to the concentrate to perform azeotropic dehydration, and diisopropyl ether was further added for crystallization to yield 16.7 g (yield: 95%) of PAG-1 of a target product as a white crystal.

### MALDI TOF-MS:

POSITIVE M⁺ 308 (corresponding to C₁₉H₁₄NO₂S⁺)

NEGATIVE M⁻ 477 (corresponding to C₂₃H₁₃F₄O₅S⁻)

### [Examples 1-2 to 1-9] Synthesis of Onium Salt PAG-2 to PAG-9

Onium salts PAG-2 to PAG-9, represented by formulae below, were synthesized using corresponding raw materials and known organic synthesis reactions.

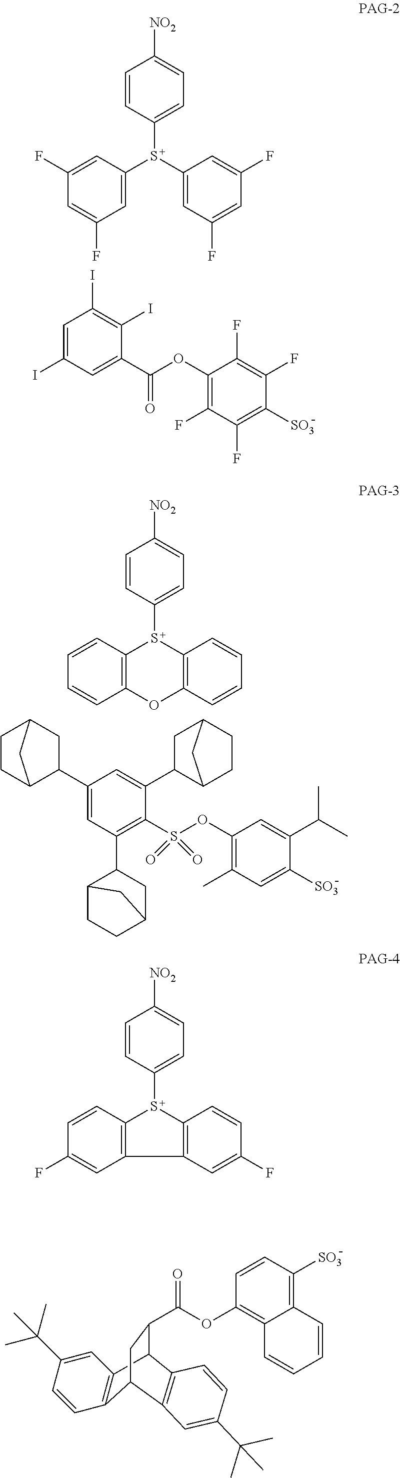

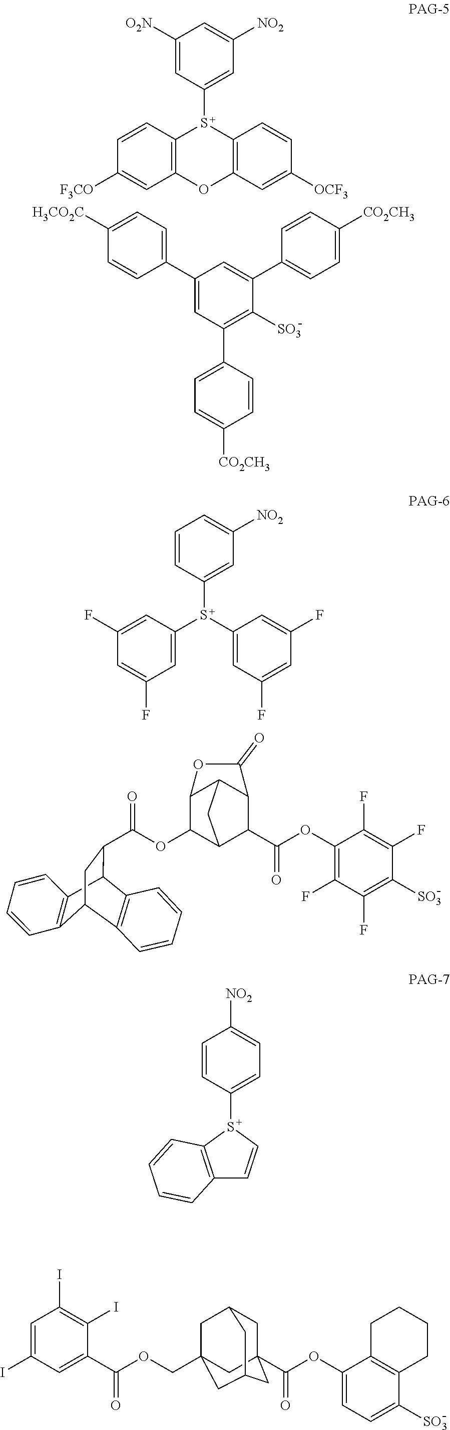

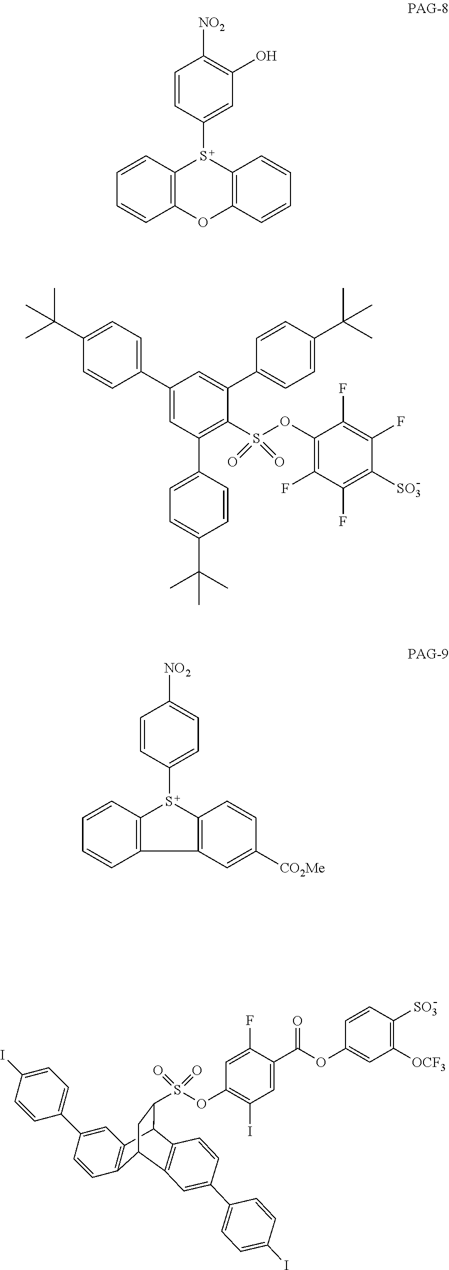

### [2] Synthesis of Base Polymer

### [Synthesis Examples 1 to 5] Synthesis of Base Polymer

### (P-1 to P-5)

Monomers were combined and subjected to a copolymerization reaction in the solvent MEK, and the reaction solution was then poured into hexane. The precipitated solid was washed with hexane, then isolated, and dried to yield base polymers (P-1 to P-5) having the compositions shown below. The resulting base polymer was analyzed for composition by ¹H-NMR, and for Mw and Mw/Mn by GPC (solvent: THF, standard: polystyrene).

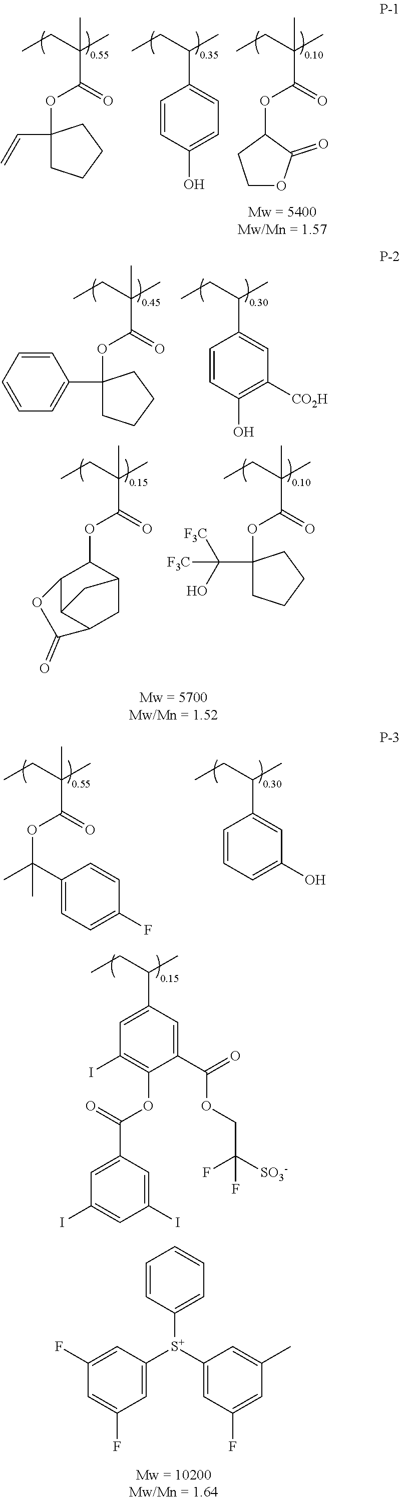

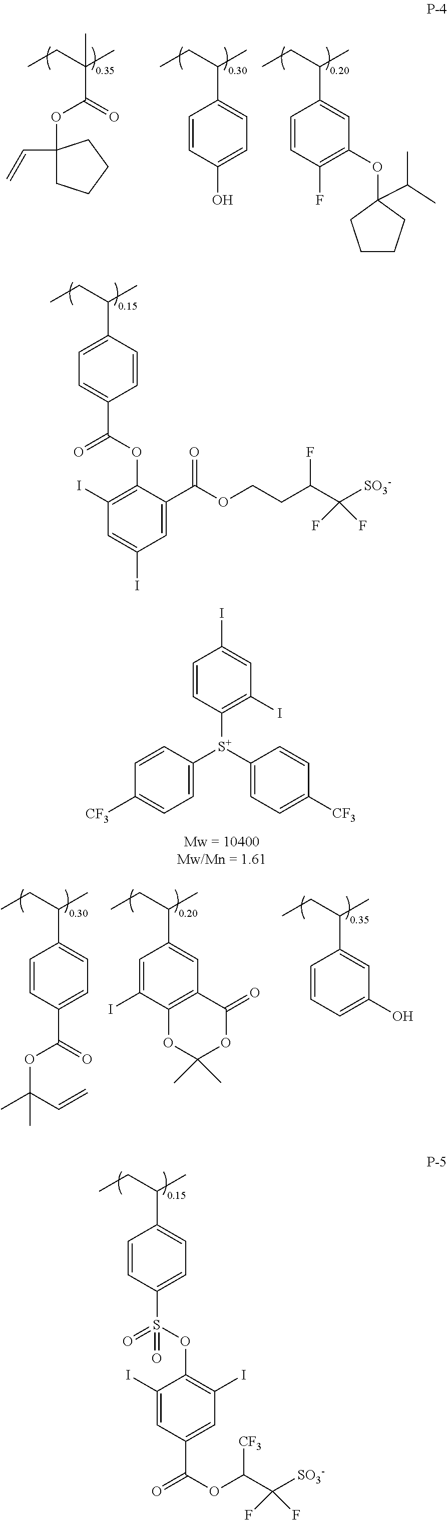

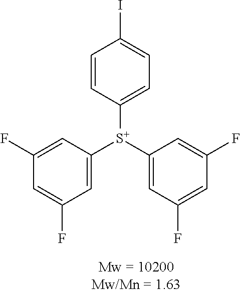 [SMILES: Fc1cc(F)cc([S+](c2ccc(I)cc2)c2cc(F)cc(F)c2)c1]

### [3] Preparation of Chemically Amplified Resist Composition

### Examples 2-1 to 2-30 and Comparative Examples 1-1 to 1-20

Photoacid generators (PAG-1 to PAG-9) composed of the onium salt of the present invention, comparative photoacid generators (PAG-A to PAG-E), other photoacid generator (PAG-X), base polymers (P-i to P-5), and quenchers (Q-1 to Q-4) were dissolved in a solvent containing 0.01% by mass of surfactant A (Omnova Solutions Inc.) according to the composition shown in Tables 1 and 2 below to prepare solutions, and the solutions were filtered through a 0.2 μm Teflon (registered trademark)-type filter to prepare chemically amplified resist compositions (R-1 to R-30 and CR-1 to CR-20).

TABLE 1

| | | Base | Photoacid | Other | | | | |
|---|---|---|---|---|---|---|---|---|
| | | polymer | generator | photoacid | Quencher | Solvent 1 | Solvent 2 | Solvent 3 |
| | Resist | (part by | (part by | generator | (part by | (part by | (part by | (part by |
| | composition | mass) | mass) | (part by mass) | mass) | mass) | mass) | mass) |
| Example 2-1 | R-1 | P-1(80) | PAG-1(28) | — | Q-1(8.0) | PGMEA(2250) | EL(2800) | DAA(550) |
| Example 2-2 | R-2 | P-1(80) | PAG-2(28) | — | Q-1(7.8) | PGMEA(2250) | EL(2800) | DAA(550) |
| Example 2-3 | R-3 | P-1(80) | PAG-3(29) | — | Q-1(7.4) | PGMEA(2250) | EL(2800) | DAA(550) |
| Example 2-4 | R-4 | P-1(80) | PAG-4(27) | — | Q-1(8.0) | PGMEA(2250) | EL(2800) | DAA(550) |
| Example 2-5 | R-5 | P-1(80) | PAG-5(15) | PAG-X(10) | Q-1(8.0) | PGMEA(2250) | EL(2800) | DAA(550) |
| Example 2-6 | R-6 | P-1(80) | PAG-6(28) | — | Q-1(8.0) | PGMEA(2250) | EL(2800) | DAA(550) |
| Example 2-7 | R-7 | P-1(80) | PAG-7(28) | — | Q-1(7.8) | PGMEA(2250) | EL(2800) | DAA(550) |
| Example 2-8 | R-8 | P-1(80) | PAG-8(29) | — | Q-1(7.4) | PGMEA(2250) | EL(2800) | DAA(550) |
| Example 2-9 | R-9 | P-1(80) | PAG-9(15) | PAG-X(8) | Q-1(8.0) | PGMEA(2250) | EL(2800) | DAA(550) |
| Example 2-10 | R-10 | P-2(80) | PAG-1(29) | — | Q-1(8.0) | PGMEA(2250) | EL(2800) | DAA(550) |
| Example 2-11 | R-11 | P-2(80) | PAG-2(30) | — | Q-3(7.8) | PGMEA(2250) | EL(2800) | DAA(550) |
| Example 2-12 | R-12 | P-2(80) | PAG-4(28) | — | Q-1(8.0) | PGMEA(2250) | EL(2800) | DAA(550) |
| Example 2-13 | R-13 | P-2(80) | PAG-5(18) | PAG-X(7) | Q-1(8.0) | PGMEA(2250) | EL(2800) | DAA(550) |
| Example 2-14 | R-14 | P-2(80) | PAG-7(28) | — | Q-2(4.0) | PGMEA(2250) | EL(2800) | DAA(550) |
| | | | | | Q-4(4.0) | | | |
| Example 2-15 | R-15 | P-3(80) | PAG-1(10) | — | Q-1(8.0) | PGMEA(2250) | EL(2800) | DAA(550) |
| Example 2-16 | R-16 | P-3(80) | PAG-2(9) | — | Q-3(7.6) | PGMEA(2250) | EL(2800) | DAA(550) |
| Example 2-17 | R-17 | P-3(80) | PAG-5(8) | PAG-X(4) | Q-1(7.8) | PGMEA(2250) | EL(2800) | DAA(550) |
| Example 2-18 | R-18 | P-3(80) | PAG-7(9) | — | Q-3(8.0) | PGMEA(2250) | EL(2800) | DAA(550) |
| Example 2-19 | R-19 | P-3(80) | PAG-9(8) | PAG-X(4) | Q-2(8.0) | PGMEA(2250) | EL(2800) | DAA(550) |
| Example 2-20 | R-20 | P-4(80) | PAG-1(10) | — | Q-1(8.0) | PGMEA(2250) | EL(2800) | DAA(550) |
| Example 2-21 | R-21 | P-4(80) | PAG-3(10) | — | Q-1(7.6) | PGMEA(2250) | EL(2800) | DAA(550) |
| Example 2-22 | R-22 | P-4(80) | PAG-4(8) | PAG-X(4) | Q-3(8.0) | PGMEA(2250) | EL(2800) | DAA(550) |
| Example 2-23 | R-23 | P-4(80) | PAG-6(10) | — | Q-2(8.0) | PGMEA(2250) | EL(2800) | DAA(550) |
| Example 2-24 | R-24 | P-4(80) | PAG-8(8) | — | Q-3(4.0) | PGMEA(2250) | EL(2800) | DAA(550) |
| | | | | | Q-4(4.0) | | | |
| Example 2-25 | R-25 | P-5(80) | PAG-1(10) | — | Q-1(8.0) | PGMEA(2250) | EL(2800) | DAA(550) |
| Example 2-26 | R-26 | P-5(80) | PAG-2(8) | — | Q-2(8.0) | PGMEA(2250) | EL(2800) | DAA(550) |
| Example 2-27 | R-27 | P-5(80) | PAG-4(8) | — | Q-3(7.8) | PGMEA(2250) | EL(2800) | DAA(550) |
| Example 2-28 | R-28 | P-5(80) | PAG-6(10) | — | Q-1(4.0) | PGMEA(2250) | EL(2800) | DAA(550) |
| | | | | | Q-4(4.0) | | | |
| Example 2-29 | R-29 | P-5(80) | PAG-7(10) | — | Q-3(8.0) | PGMEA(2250) | EL(2800) | DAA(550) |
| Example 2-30 | R-30 | P-5(80) | PAG-9(8) | PAG-X(4) | Q-2(7.6) | PGMEA(2250) | EL(2800) | DAA(550) |

TABLE 2

| | | | | Other | | | | |
|---|---|---|---|---|---|---|---|---|
| | | Base | Photoacid | photoacid | | | | |
| | | polymer | generator | generator | Quencher | Solvent 1 | Solvent 2 | Solvent 3 |
| | Resist | (part by | (part by | (part by | (part by | (part by | (part by | (part by |
| | composition | mass) | mass) | mass) | mass) | mass) | mass) | mass) |
| Comparative | CR-1 | P-1(80) | PAG-A(28) | — | Q-1(8.0) | PGMEA(2250) | EL(2800) | DAA(550) |
| Example 1-1 | | | | | | | | |
| Comparative | CR-2 | P-1(80) | PAG-B(28) | — | Q-1(7.8) | PGMEA(2250) | EL(2800) | DAA(550) |
| Example 1-2 | | | | | | | | |
| Comparative | CR-3 | P-1(80) | PAG-C(29) | — | Q-1(7.8) | PGMEA(2250) | EL(2800) | DAA(550) |
| Example 1-3 | | | | | | | | |
| Comparative | CR-4 | P-1(80) | PAG-D(27) | — | Q-1(8.2) | PGMEA(2250) | EL(2800) | DAA(550) |
| Example 1-4 | | | | | | | | |
| Comparative | CR-5 | P-1(80) | PAG-E(15) | PAG-X(10) | Q-1(8.2) | PGMEA(2250) | EL(2800) | DAA(550) |
| Example 1-5 | | | | | | | | |
| Comparative | CR-6 | P-2(80) | PAG-A(27) | — | Q-2(8.0) | PGMEA(2250) | EL(2800) | DAA(550) |
| Example 1-6 | | | | | | | | |
| Comparative | CR-7 | P-2(80) | PAG-B(25) | — | Q-1(8.0) | PGMEA(2250) | EL(2800) | DAA(550) |
| Example 1-7 | | | | | | | | |
| Comparative | CR-8 | P-2(80) | PAG-E(18) | PAG-X(7) | Q-1(8.0) | PGMEA(2250) | EL(2800) | DAA(550) |
| Example 1-8 | | | | | | | | |
| Comparative | CR-9 | P-3(80) | PAG-A(10) | — | Q-1(7.8) | PGMEA(2250) | EL(2800) | DAA(550) |
| Example 1-9 | | | | | | | | |
| Comparative | CR-10 | P-3(80) | PAG-B(9) | — | Q-3(8.0) | PGMEA(2250) | EL(2800) | DAA(550) |
| Example 1-10 | | | | | | | | |
| Comparative | CR-11 | P-3(80) | PAG-C(10) | — | Q-1(4.0) | PGMEA(2250) | EL(2800) | DAA(550) |
| Example 1-11 | | | | | Q-4(4.0) | | | |
| Comparative | CR-12 | P-3(80) | PAG-D(8) | — | Q-2(7.6) | PGMEA(2250) | EL(2800) | DAA(550) |
| Example 1-12 | | | | | | | | |
| Comparative | CR-13 | P-4(80) | PAG-A(10) | — | Q-1(7.8) | PGMEA(2250) | EL(2800) | DAA(550) |
| Example 1-13 | | | | | | | | |
| Comparative | CR-14 | P-4(80) | PAG-B(10) | — | Q-2(8.0) | PGMEA(2250) | EL(2800) | DAA(550) |
| Example 1-14 | | | | | | | | |
| Comparative | CR-15 | P-4(80) | PAG-C(10) | — | Q-3(4.0) | PGMEA(2250) | EL(2800) | DAA(550) |
| Example 1-15 | | | | | Q-4(4.0) | | | |
| Comparative | CR-16 | P-4(80) | PAG-E(8) | PAG-X(4) | Q-1(8.0) | PGMEA(2250) | EL(2800) | DAA(550) |
| Example 1-16 | | | | | | | | |
| Comparative | CR-17 | P-5(80) | PAG-A(10) | — | Q-1(8.0) | PGMEA(2250) | EL(2800) | DAA(550) |
| Example 1-17 | | | | | | | | |
| Comparative | CR-18 | P-5(80) | PAG-B(10) | — | Q-3(7.6) | PGMEA(2250) | EL(2800) | DAA(550) |
| Example 1-18 | | | | | | | | |
| Comparative | CR-19 | P-5(80) | PAG-C(8) | — | Q-1(4.0) | PGMEA(2250) | EL(2800) | DAA(550) |
| Example 1-19 | | | | | Q-4(4.0) | | | |
| Comparative | CR-20 | P-5(80) | PAG-E(8) | PAG-X(4) | Q-2(8.0) | PGMEA(2250) | EL(2800) | DAA(550) |
| Example 1-20 | | | | | | | | |

The solvents, other photoacid generator PAG-X, comparative photoacid generators PAG-A to PAG-E, quenchers Q-1 to Q-4, and surfactant A in Tables 1 and 2 are as described below.

### Solvent: PGMEA (Propylene Glycol Monomethyl Ether Acetate)

EL (ethyl lactate)
DAA (diacetone alcohol)

### Another Photoacid Generator: PAG-X

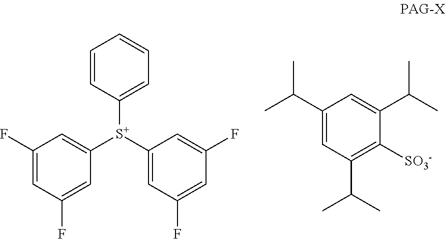

### Comparative Photoacid Generators: PAG-A to PAG-E

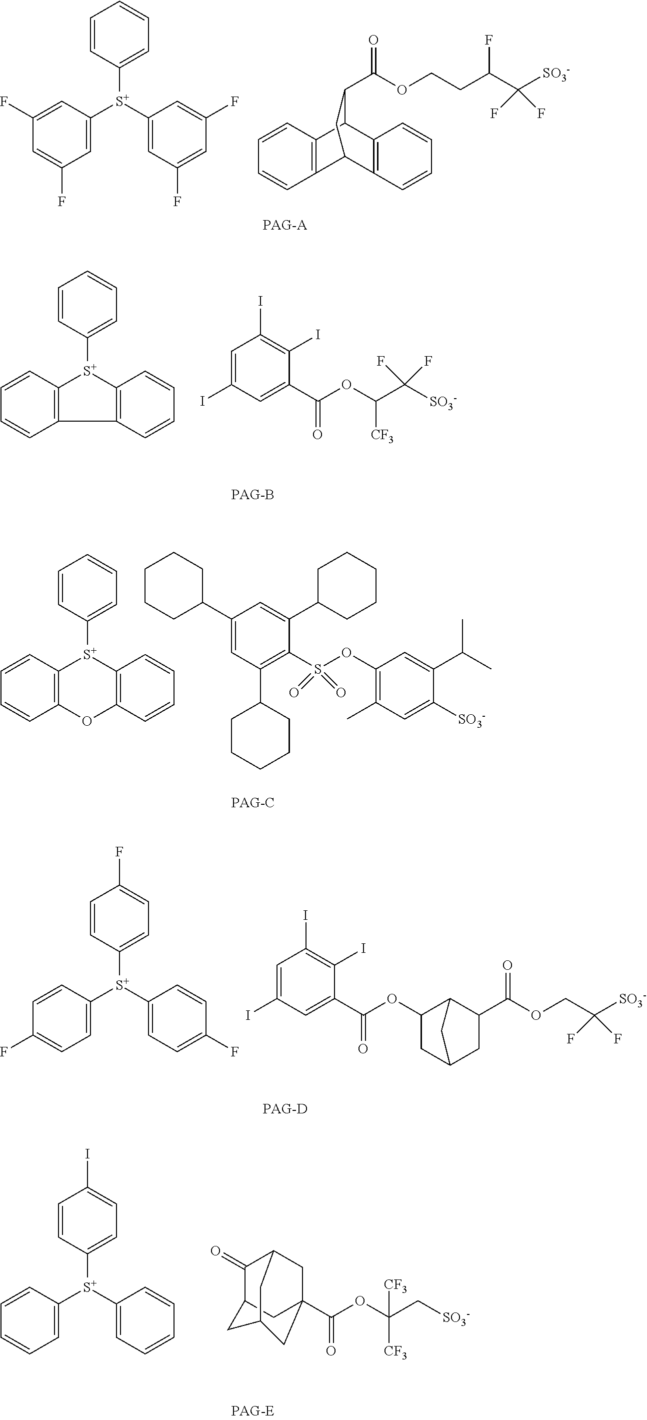

### Quencher: Q-1 to Q-4

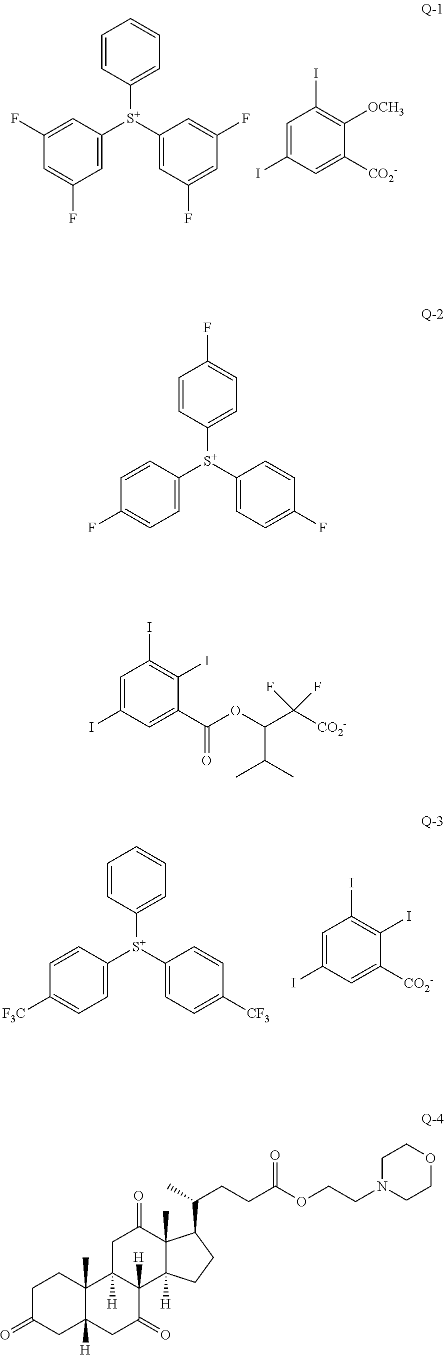

Surfactant A: 3-methyl-3-(2,2,2-trifluoroethoxymethyl)oxetane/tetrahydrofuran/2,2-dimethyl-1,3-propanediol copolymer (Omnova Solutions, Inc.)

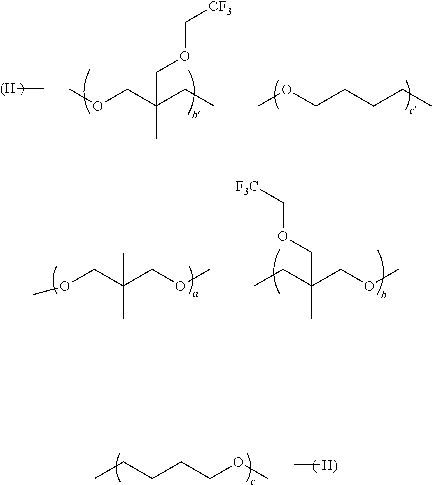

a:(b+b′):(c+c′)=1:4 to 7:0.01 to 1 (molar ratio)

Mw=1500

### [4] Evaluation of EUV Lithography (1)

### Examples 3-1 to 3-30 and Comparative Examples 2-1 to 2-20

The chemically amplified resist compositions (R-1 to R-30 and CR-1 to CR-20) in Tables 1 and 2 were each spin-coated onto a Si substrate on which a silicon-containing spin-on hard mask SHB-A940 (silicon content of 43% by mass), manufactured by Shin-Etsu Chemical Co., Ltd., was formed with a film thickness of 20 nm, and prebaked using a hot plate at 100° C. for 60 seconds to prepare a resist film having a thickness of 50 nm. Using an EUV scanner NXE3400 (NA 0.33, o 0.9/0.6, dipole illumination) manufactured by ASML, the resist film was exposed through a mask bearing a LS pattern with a size of 18 nm and a pitch of 36 nm on a wafer while the exposure dose and focus was varied (exposure pitch: 1 mJ/cm², focus pitch: 0.020 μm). After the exposure, PEB was performed for 60 seconds at temperatures shown in Tables 3 and 4. Thereafter, the resist film was puddle developed in a 2.38% by mass TMAH aqueous solution for 30 seconds, rinsed with a surfactant-containing rinsing material, and spin-dried to form a positive pattern.

The resulting LS pattern was observed under CD-SEM (CG6300) manufactured by Hitachi High-Tech Corporation to evaluate sensitivity, EL, LWR, depth of focus (DOF), and collapse limit according to methods described below. In addition, the development defects of the resulting LS pattern were evaluated. The results are shown in Tables 3 and 4.

### [Evaluation of Sensitivity]

The optimum exposure dose Eₒₚ(mJ/cm²) that provided an LS pattern with a line width of 18 nm and a pitch of 36 nm was determined as an index of sensitivity. The smaller the value, the higher the sensitivity.

### [Evaluation of EL]

Based on the exposure dose that produced the LS pattern with a space width within a range of ±10% of 18 nm (i.e., 16.2 to 19.8 nm), EL (unit: %) was determined using an equation below. The greater the value, the better the performance.

$$EL ( \% ) = ( \vert {E}_{1} - {E}_{2} \vert / {E}_{op} ) \times 100$$

E₁: optimum exposure dose that provided an LS pattern with a line width of 16.2 nm and a pitch of 36 nm
E₂: optimum exposure dose that provided an LS pattern with a line width of 19.8 nm and a pitch of 36 nm
Eₒₚ: optimum exposure dose that provided an LS pattern with a line width of 18 nm and a pitch of 36 nm

### [Evaluation of LWR]

For the LS pattern formed by exposure at Eₒₚ, the line width was measured at longitudinally spaced apart 10 points, from which a 3-fold value (3σ) of standard deviation (σ) was determined as LWR. A smaller value of 3σ indicates a pattern with small roughness and uniform line width.

### [Evaluation of DOF]

To evaluate the depth of focus, a range of focus that provided the LS pattern with a size of 18 nm±10% (i.e., 16.2 to 19.8 nm) was determined. The greater the value, the wider the depth of focus.

### [Evaluation of Collapse Limit of Line Pattern]

For each exposure dose at the optimum focus of the LS pattern, the line width was measured at longitudinally spaced apart 10 points. The smallest line width obtained without collapse was defined as the collapse limit. The smaller the value, the better the collapse limit.

### [Evaluation of Development Defect]

Using defect inspection apparatus KLA 2360 (trade name), manufactured by KLA-Tencor Corporation, an LS pattern with a line width of 18 nm and a pitch of 36 nm formed at the optimum exposure dose was measured by setting a pixel size of the defect inspection apparatus to 0.16 μm and a threshold value to 20 to detect defects (defects/cm²) extracted from a difference produced at the time of superposing pixel units on a comparison image, and the number of the defects per unit area (defects/cm²) was calculated. Subsequently, a defect review was conducted to classify and extract development defects from all the defects detected, and the number of development defects per unit area (defects/cm²) was calculated. The value of less than 0.5 was designated as A, 0.5 or more and less than 1.0 as B, 1.0 or more and less than 5.0 as C, and 5.0 or more as D. A smaller value thereof indicates better performance.

TABLE 3

| | | PEB | | | | | | |
|---|---|---|---|---|---|---|---|---|
| | | temper- | Optimum | | | | Collapse | Develop- |
| | Resist | ature | exposure | EL | LWR | DOF | limit | ment |
| | composition | (° C.) | (mJ/cm²) | (%) | (nm) | (nm) | (nm) | defect |
| Example 3-1 | R-1 | 100 | 32 | 19 | 2.3 | 120 | 10.5 | A |
| Example 3-2 | R-2 | 100 | 33 | 18 | 2.4 | 120 | 10.7 | A |
| Example 3-3 | R-3 | 95 | 33 | 17 | 2.4 | 120 | 10.9 | A |
| Example 3-4 | R-4 | 105 | 33 | 17 | 2.5 | 110 | 10.8 | A |
| Example 3-5 | R-5 | 105 | 34 | 18 | 2.5 | 100 | 10.5 | A |
| Example 3-6 | R-6 | 95 | 32 | 19 | 2.5 | 110 | 11.0 | A |
| Example 3-7 | R-7 | 105 | 33 | 17 | 2.5 | 120 | 11.2 | A |
| Example 3-8 | R-8 | 105 | 33 | 18 | 2.5 | 120 | 11.1 | A |
| Example 3-9 | R-9 | 100 | 34 | 18 | 2.5 | 120 | 10.7 | A |
| Example 3-10 | R-10 | 95 | 35 | 17 | 2.4 | 120 | 10.7 | A |
| Example 3-11 | R-11 | 100 | 35 | 18 | 2.6 | 100 | 10.7 | A |
| Example 3-12 | R-12 | 105 | 34 | 19 | 2.4 | 110 | 11.1 | A |
| Example 3-13 | R-13 | 105 | 33 | 18 | 2.4 | 110 | 10.8 | A |
| Example 3-14 | R-14 | 100 | 34 | 19 | 2.5 | 110 | 11.4 | A |
| Example 3-15 | R-15 | 100 | 35 | 18 | 2.4 | 120 | 10.8 | A |
| Example 3-16 | R-16 | 100 | 34 | 17 | 2.5 | 120 | 10.7 | A |
| Example 3-17 | R-17 | 105 | 32 | 18 | 2.3 | 110 | 11.2 | A |
| Example 3-18 | R-18 | 95 | 33 | 18 | 2.4 | 120 | 10.9 | A |
| Example 3-19 | R-19 | 100 | 35 | 17 | 2.5 | 110 | 10.8 | A |
| Example 3-20 | R-20 | 105 | 34 | 18 | 2.4 | 100 | 11.1 | A |
| Example 3-21 | R-21 | 95 | 34 | 17 | 2.5 | 120 | 11.1 | A |
| Example 3-22 | R-22 | 100 | 33 | 19 | 2.4 | 110 | 10.9 | A |
| Example 3-23 | R-23 | 105 | 33 | 18 | 2.5 | 120 | 11.3 | A |
| Example 3-24 | R-24 | 95 | 34 | 18 | 2.3 | 120 | 11.2 | A |
| Example 3-25 | R-25 | 100 | 35 | 18 | 2.5 | 110 | 10.9 | A |
| Example 3-26 | R-26 | 95 | 32 | 19 | 2.5 | 120 | 10.9 | A |
| Example 3-27 | R-27 | 100 | 34 | 18 | 2.4 | 100 | 10.8 | A |
| Example 3-28 | R-28 | 100 | 33 | 17 | 2.3 | 120 | 11.1 | A |
| Example 3-29 | R-29 | 100 | 34 | 17 | 2.4 | 110 | 11.2 | A |
| Example 3-30 | R-30 | 95 | 34 | 19 | 2.5 | 110 | 11.0 | A |

TABLE 4

| | | PEB | | | | | | |
|---|---|---|---|---|---|---|---|---|
| | | temper- | Optimum | | | | Collapse | Develop- |
| | Resist | ature | exposure | EL | WR | DOF | limit | ment |
| | composition | (° C.) | (mJ/cm²) | (%) | (nm) | (nm) | (nm) | defect |
| Comparative | CR-1 | 100 | 37 | 14 | 3.1 | 80 | 11.9 | B |
| Example 2-1 | | | | | | | | |
| Comparative | CR-2 | 100 | 38 | 14 | 2.9 | 90 | 12.1 | C |
| Example 2-2 | | | | | | | | |
| Comparative | CR-3 | 105 | 40 | 15 | 3.0 | 100 | 12.3 | B |
| Example 2-3 | | | | | | | | |
| Comparative | CR-4 | 100 | 41 | 15 | 3.1 | 80 | 12.4 | C |
| Example 2-4 | | | | | | | | |
| Comparative | CR-5 | 100 | 41 | 14 | 3.0 | 70 | 12.4 | B |
| Example 2-5 | | | | | | | | |
| Comparative | CR-6 | 100 | 37 | 13 | 2.8 | 80 | 12.3 | B |
| Example 2-6 | | | | | | | | |
| Comparative | CR-7 | 100 | 39 | 15 | 3.0 | 90 | 12.2 | C |
| Example 2-7 | | | | | | | | |
| Comparative | CR-8 | 105 | 39 | 14 | 3.1 | 80 | 12.3 | C |
| Example 2-8 | | | | | | | | |
| Comparative | CR-9 | 100 | 40 | 13 | 3.0 | 100 | 12.1 | B |
| Example 2-9 | | | | | | | | |
| Comparative | CR-10 | 100 | 41 | 13 | 3.1 | 90 | 12.4 | C |
| Example 2-10 | | | | | | | | |
| Comparative | CR-11 | 95 | 40 | 14 | 3.1 | 80 | 12.5 | B |
| Example 2-11 | | | | | | | | |
| Comparative | CR-12 | 100 | 38 | 15 | 2.7 | 80 | 12.1 | C |
| Example 2-12 | | | | | | | | |
| Comparative | CR-13 | 100 | 38 | 16 | 2.9 | 70 | 12.1 | B |
| Example 2-13 | | | | | | | | |
| Comparative | CR-14 | 100 | 39 | 15 | 2.7 | 90 | 11.9 | C |
| Example 2-14 | | | | | | | | |
| Comparative | CR-15 | 100 | 38 | 14 | 2.8 | 80 | 12.1 | B |
| Example 2-15 | | | | | | | | |
| Comparative | CR-16 | 95 | 40 | 15 | 2.9 | 90 | 11.9 | C |
| Example 2-16 | | | | | | | | |
| Comparative | CR-17 | 100 | 38 | 13 | 2.7 | 80 | 12.2 | B |
| Example 2-17 | | | | | | | | |
| Comparative | CR-18 | 105 | 38 | 15 | 3.0 | 80 | 11.7 | B |
| Example 2-18 | | | | | | | | |
| Comparative | CR-19 | 100 | 37 | 14 | 2.9 | 90 | 11.8 | C |
| Example 2-19 | | | | | | | | |
| Comparative | CR-20 | 100 | 38 | 15 | 2.8 | 80 | 12.2 | B |
| Example 2-20 | | | | | | | | |

From the results shown in Tables 3 and 4, it was found that the chemically amplified resist composition containing the photoacid generator composed of an onium salt of the present invention had good sensitivity and excellent EL, LWR, and DOF. In addition, it was confirmed that the value of the collapse limit was small and the pattern collapse resistance was high even in the fine patterning. Furthermore, it was confirmed that development defects are also suppressed. Therefore, the chemically-amplified resist composition of the present invention has been demonstrated to be suitable as a material for EUV lithography.

### [5] Evaluation of EUV Lithography (2)

### Examples 4-1 to 4-30 and Comparative Examples 3-1 to 3-20

The chemically amplified resist compositions (R-1 to R-30 and CR-1 to CR-20) in Tables 1 and 2 were spin-coated onto a Si substrate on which a silicon-containing spin-on hard mask SHB-A940 (silicon content of 43% by mass), manufactured by Shin-Etsu Chemical Co., Ltd., was formed with a film thickness of 20 nm, and prebaked using a hot plate at 105° C. for 60 seconds to prepare a resist film having a film thickness of 50 nm. Using an EUV scanner NXE3400 (NA 0.33, o 0.9/0.6, quadrupole illumination, mask bearing a hole pattern at a pitch 46 nm on-wafer size and +20% bias) manufactured by ASML, the resist film was exposed, baked (PEB) on a hot plate at the temperature shown in Tables 5 and 6 for 60 seconds, and developed in a 2.38% by mass TMAH aqueous solution for 30 seconds to form a hole pattern with a size of 23 nm.

Using CD-SEM (CG6300) manufactured by Hitachi High-Tech Corporation, the exposure dose that produced a hole pattern with a size of 23 nm was measured and defined as sensitivity. The size of 50 holes herein was measured to define a 3-fold value (3o) of standard deviation (a) calculated from the results as CDU. The results are shown in Tables 5 and 6.

TABLE 5

| | | PEB | Optimum | |
|---|---|---|---|---|
| | Resist | temperature | exposure | CDU |
| | composition | (° C.) | (mJ/cm²) | (nm) |
| Example 4-1 | R-1 | 95 | 22 | 2.2 |
| Example 4-2 | R-2 | 95 | 23 | 2.3 |
| Example 4-3 | R-3 | 95 | 25 | 2.4 |
| Example 4-4 | R-4 | 90 | 24 | 2.3 |
| Example 4-5 | R-5 | 90 | 23 | 2.4 |
| Example 4-6 | R-6 | 95 | 24 | 2.4 |
| Example 4-7 | R-7 | 90 | 24 | 2.3 |
| Example 4-8 | R-8 | 95 | 25 | 2.2 |
| Example 4-9 | R-9 | 90 | 23 | 2.3 |
| Example 4-10 | R-10 | 95 | 24 | 2.4 |
| Example 4-11 | R-11 | 90 | 23 | 2.3 |
| Example 4-12 | R-12 | 90 | 24 | 2.5 |
| Example 4-13 | R-13 | 95 | 24 | 2.4 |
| Example 4-14 | R-14 | 95 | 25 | 2.5 |
| Example 4-15 | R-15 | 95 | 23 | 2.4 |
| Example 4-16 | R-16 | 95 | 23 | 2.3 |
| Example 4-17 | R-17 | 90 | 22 | 2.4 |
| Example 4-18 | R-18 | 90 | 22 | 2.3 |
| Example 4-19 | R-19 | 95 | 23 | 2.3 |
| Example 4-20 | R-20 | 90 | 23 | 2.5 |
| Example 4-21 | R-21 | 95 | 24 | 2.3 |
| Example 4-22 | R-22 | 90 | 24 | 2.4 |
| Example 4-23 | R-23 | 95 | 23 | 2.3 |
| Example 4-24 | R-24 | 90 | 25 | 2.2 |
| Example 4-25 | R-25 | 95 | 22 | 2.4 |
| Example 4-26 | R-26 | 90 | 23 | 2.3 |
| Example 4-27 | R-27 | 95 | 24 | 2.4 |
| Example 4-28 | R-28 | 90 | 23 | 2.4 |
| Example 4-29 | R-29 | 90 | 25 | 2.3 |
| Example 4-30 | R-30 | 95 | 24 | 2.3 |

TABLE 6

| | | PEB | Optimum | |
|---|---|---|---|---|
| | Resist | temperature | exposure | CDU |
| | composition | (° C.) | (mJ/cm²) | (nm) |
| Comparative | CR-1 | 90 | 28 | 2.9 |
| Example 3-1 | | | | |
| Comparative | CR-2 | 90 | 29 | 2.8 |
| Example 3-2 | | | | |
| Comparative | CR-3 | 85 | 27 | 2.8 |
| Example 3-3 | | | | |
| Comparative | CR-4 | 90 | 28 | 2.7 |
| Example 3-4 | | | | |
| Comparative | CR-5 | 95 | 30 | 3.1 |
| Example 3-5 | | | | |
| Comparative | CR-6 | 90 | 28 | 3.0 |
| Example 3-6 | | | | |
| Comparative | CR-7 | 85 | 28 | 2.8 |
| Example 3-7 | | | | |
| Comparative | CR-8 | 90 | 29 | 3.1 |
| Example 3-8 | | | | |
| Comparative | CR-9 | 90 | 27 | 2.9 |
| Example 3-9 | | | | |
| Comparative | CR-10 | 90 | 29 | 2.7 |
| Example 3-10 | | | | |
| Comparative | CR-11 | 85 | 29 | 3.1 |
| Example 3-11 | | | | |
| Comparative | CR-12 | 90 | 29 | 3.2 |
| Example 3-12 | | | | |
| Comparative | CR-13 | 90 | 30 | 3.0 |
| Example 3-13 | | | | |
| Comparative | CR-14 | 90 | 31 | 3.1 |
| Example 3-14 | | | | |
| Comparative | CR-15 | 90 | 29 | 2.8 |
| Example 3-15 | | | | |
| Comparative | CR-16 | 85 | 27 | 2.7 |
| Example 3-16 | | | | |
| Comparative | CR-17 | 95 | 28 | 2.8 |
| Example 3-17 | | | | |
| Comparative | CR-18 | 90 | 27 | 2.7 |
| Example 3-18 | | | | |
| Comparative | CR-19 | 90 | 28 | 2.9 |
| Example 3-19 | | | | |
| Comparative | CR-20 | 90 | 28 | 2.8 |
| Example 3-20 | | | | |

From the results shown in Tables 5 and 6, it was confirmed that the chemically amplified resist composition containing the photoacid generator composed of an onium salt of the present invention had good sensitivity and excellent CDU.

The present description includes the following embodiments.
[1] An onium salt comprising:
an anion represented by general formula (1A); and
a cation represented by general formula (1B):

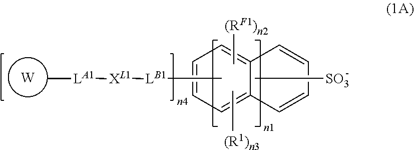

wherein “n1” is 0 or 1, “n2” is an integer of 0 to 4, “n3” is an integer of 0 to 4, provided that when “n1” is 0, 0≤n2+n3≤4, and when “n1” is 1, 0≤n2+n3≤6, and “n4” is 0 or 1;
W is a hydrocarbyl group having 6 to 60 carbon atoms and comprising at least one aromatic ring, wherein the hydrocarbyl group optionally comprises a heteroatom;
R^{F1} is a fluorine atom, a fluorinated saturated hydrocarbyl group having 1 to 6 carbon atoms, a fluorinated saturated hydrocarbyloxy group having 1 to 6 carbon atoms, or a fluorinated saturated hydrocarbylthio group having 1 to 6 carbon atoms;
R¹ is a halogen atom other than fluorine, a nitro group, a hydrocarbyl group having 1 to 20 carbon atoms and optionally comprising a heteroatom, a hydrocarbyloxy group having 1 to 20 carbon atoms and optionally comprising a heteroatom, or a hydrocarbylthio group having 1 to 20 carbon atoms and optionally comprising a heteroatom, wherein when “n3” is 2, 3, or 4, R¹ are the same as or different from each other, and a plurality of R¹ are optionally bonded to each other to form a ring together with carbon atoms to which the R¹ are bonded;
L^{A1} and L^{B1} are each independently a single bond, an ether bond, an ester bond, a sulfonate ester bond, an amide bond, a sulfonamide bond, a carbonate bond, or a carbamate bond;
X^{L1} is a single bond or a hydrocarbylene group having 1 to 40 carbon atoms and optionally comprising a heteroatom, and

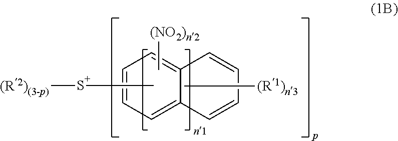

wherein “p” is 1, 2, or 3, “n′1” is 0 or 1, “n′2” is 1 or 2, and “n′3” is an integer of 0 to 6, provided that when “n′1” is 0, 1≤n′2+n′3≤5, and when “n′1” is 1, 1≤n′2+n′3≤7;
R′¹ is a halogen atom, a cyano group, a hydroxy group, a carboxy group, an alkoxycarbonyl group, a hydrocarbyl group having 1 to 20 carbon atoms and optionally comprising a heteroatom, a hydrocarbyloxy group having 1 to 20 carbon atoms and optionally comprising a heteroatom, or a hydrocarbylthio group having 1 to 20 carbon atoms and optionally comprising a heteroatom, wherein when “n′3” is 2 to 6, R′¹ are the same as or different from each other, and two R¹ are optionally bonded to each other to form a ring together with carbon atoms to which the R¹ are bonded; and
R′² is a halogen atom or a hydrocarbyl group having 1 to 30 carbon atoms and optionally comprising a heteroatom, wherein when “p” is 1, two R′² are the same as or different from each other, and two of three substituents bonded to S⁺ are optionally bonded to each other to form a ring together with a sulfur atom to which the substituents are bonded.
[2] The onium salt according to [1], wherein the W is represented by general formula (W-1) or (W-2):

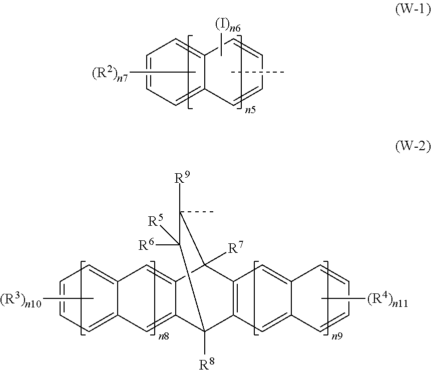

wherein “n5” is 0 or 1, “n6” is an integer of 0 to 4, “n7” is an integer of 1 to 4, provided that when “n5” is 0, 1≤n6+n7≤5, and when “n5” is 1, 1≤n6+n7≤7, “n8” is 0 or 1, “n9” is 0 or 1, “n10” is an integer of 0 to 4, and “n11” is an integer of 0 to 4;
each R² is independently a hydrogen atom, a halogen atom other than an iodine atom, or a hydrocarbyl group having 1 to 20 carbon atoms and optionally comprising a heteroatom;
R³ and R⁴ are each independently a hydrogen atom, a halogen atom, or a hydrocarbyl group having 1 to 20 carbon atoms and optionally comprising a heteroatom;
R⁵ to R⁹ are each independently a hydrogen atom, a halogen atom, or a hydrocarbyl group having 1 to 20 carbon atoms and optionally comprising a heteroatom; and
the dashed line represents a bond to L^{A1}.
[3] The onium salt according to [1] or [2], wherein the anion is represented by general formula (1A-1):

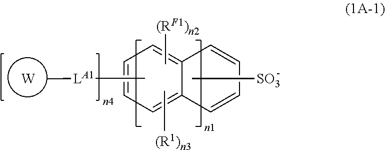

wherein “n1” to “n4”, W, R^{F1}, R¹, and L^{A1} are the same as above.
[4] The onium salt according to any one of [1] to [3], wherein the cation is represented by general formula (1B-1):

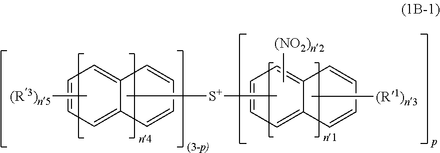

wherein “p”, “n′1” to “n′3”, and R¹ are the same as above;
“n′4” is 0 or 1, “n′5” is an integer of 0 to 5; and
R′³ is a halogen atom, a cyano group, a hydroxy group, a carboxy group, an alkoxycarbonyl group, a hydrocarbyl group having 1 to 20 carbon atoms and optionally comprising a heteroatom, a hydrocarbyloxy group having 1 to 20 carbon atoms and optionally comprising a heteroatom, or a hydrocarbylthio group having 1 to 20 carbon atoms and optionally comprising a heteroatom, wherein when “n′5” is 2 to 5, R′³ are the same as or different from each other, and two R′³ are optionally bonded to each other to form a ring together with carbon atoms to which the R′³ are bonded.
[5] A photoacid generator comprising the onium salt according to any one of [1] to [4].
[6] A chemically amplified resist composition comprising the photoacid generator according to [5].
[7] The chemically amplified resist composition according to [6], further comprising a base polymer comprising a repeating unit represented by either or both of general formulae (a1) and (a2):

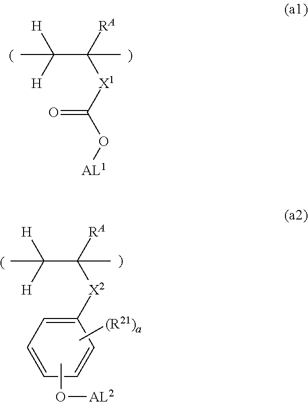

wherein each R^{A} is independently a hydrogen atom, a fluorine atom, a methyl group, or a trifluoromethyl group;
X¹ is a single bond, a phenylene group, a naphthylene group, or *—C(═O)—O—X¹¹—, wherein the phenylene group or naphthylene group is optionally substituted with a hydroxy group, a nitro group, a cyano group, a saturated hydrocarbyl group having 1 to 10 carbon atoms and optionally comprising a fluorine atom, a saturated hydrocarbyloxy group having 1 to 10 carbon atoms and optionally comprising a fluorine atom, or a halogen atom; X¹¹ is a saturated hydrocarbylene group having 1 to 10 carbon atoms, a phenylene group, or a naphthylene group, wherein the saturated hydrocarbylene group optionally comprises a hydroxy group, an ether bond, an ester bond, or a lactone ring;
X² is a single bond or *—C(═O)—O—;
“*” represents a bond to a carbon atom of a main chain;
R²¹ is a halogen atom, a cyano group, a hydroxy group, a nitro group, a hydrocarbyl group having 1 to 20 carbon atoms and optionally comprising a heteroatom, a hydrocarbyloxy group having 1 to 20 carbon atoms and optionally comprising a heteroatom, a hydrocarbylcarbonyl group having 2 to 20 carbon atoms and optionally comprising a heteroatom, a hydrocarbylcarbonyloxy group having 2 to 20 carbon atoms and optionally comprising a heteroatom, or a hydrocarbyloxycarbonyl group having 2 to 20 carbon atoms and optionally comprising a heteroatom;
AL¹ and AL² are each independently an acid labile group; and
“a” is an integer of 0 to 4.
[8] The chemically amplified resist composition according to [6] or [7], wherein the base polymer further comprises a repeating unit represented by general formula (a3):

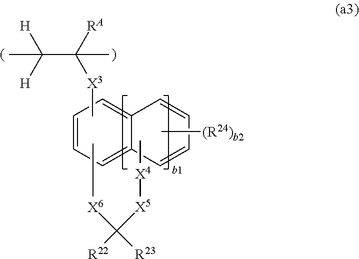

wherein “b1” is 0 or 1, “b2” is an integer of 0 to 3 when the “b1” is 0, and an integer of 0 to 5 when the “b1” is 1;
R^{A} is a hydrogen atom, a fluorine atom, a methyl group, or a trifluoromethyl group;
X³ is a single bond, *—C(═O)—O—, or *—C(═O)—NH—, and “*” represents a bond to a carbon atom of a main chain;
X⁴ is a single bond, an aliphatic hydrocarbylene group having 1 to 4 carbon atoms, a carbonyl group, a sulfonyl group, or a group obtained by combining these groups;
X⁵ and X⁶ are each independently an oxygen atom, or a sulfur atom, provided that X⁴ and X⁶ are bonded to adjacent carbon atoms of an aromatic ring.

R²² and R²³ are each independently a hydrogen atom or a hydrocarbyl group having 1 to 20 carbon atoms and optionally comprising a heteroatom, or R²² and R²³ are optionally bonded to each other to form a ring together with a carbon atom to which the R²² and R²³ are bonded; and
R²⁴ is a halogen atom, a hydroxy group, a cyano group, a nitro group, a hydrocarbyl group having 1 to 20 carbon atoms and optionally comprising a heteroatom, a hydrocarbyloxy group having 1 to 20 carbon atoms and optionally comprising a heteroatom, a hydrocarbyloxycarbonyl group having 2 to 20 carbon atoms and optionally comprising a heteroatom, a hydrocarbylthio group having 1 to 20 carbon atoms and optionally comprising a heteroatom, or —N(R^{24A})(R^{24B}) where R^{24A} and R^{24B} are each independently a hydrogen atom or a hydrocarbyl group having 1 to 6 carbon atoms, and when “b2” is 2 or more, a plurality of R²⁴ are optionally bonded to each other to form a ring together with carbon atoms of an aromatic ring to which the R²⁴ are bonded.
[9] The chemically amplified resist composition according to any one of [6] to [8], wherein the base polymer further comprises a repeating unit represented by either or both of general formulae (b1) and (b2):

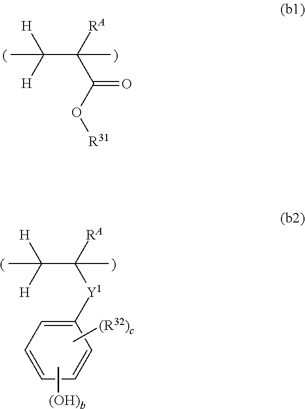

wherein each R^{A} is independently a hydrogen atom, a fluorine atom, a methyl group, or a trifluoromethyl group;
Y¹ is a single bond or *—C(═O)—O—, and represents a bond to a carbon atom of a main chain;
R³¹ is a hydrogen atom or a group having 1 to 20 carbon atoms and comprising at least one structure selected from the group consisting of a hydroxy group other than a phenolic hydroxy group, a cyano group, a carbonyl group, a carboxy group, an ether bond, an ester bond, a sulfonate ester bond, a carbonate bond, a lactone ring, a sultone ring, and a carboxylic anhydride (—C(═O)—O—C(═O)—);
R³² is a halogen atom, a carboxy group, a nitro group, a cyano group, an alkoxycarbonyl group, a hydrocarbyl group having 1 to 20 carbon atoms and optionally comprising a heteroatom, a hydrocarbyloxy group having 1 to 20 carbon atoms and optionally comprising a heteroatom, a hydrocarbylcarbonyl group having 2 to 20 carbon atoms and optionally comprising a heteroatom, a hydrocarbylcarbonyloxy group having 2 to 20 carbon atoms and optionally comprising a heteroatom, or a hydrocarbyloxycarbonyl group having 2 to 20 carbon atoms and optionally comprising a heteroatom, wherein when “c” is 2 or more, R³² are the same as or different from each other; and
“b” is an integer of 1 to 4, and “c” is an integer of 0 to 4, provided that 1≤b+c≤5.
[10] The chemically amplified resist composition according to any one of [6] to [9], wherein the base polymer further comprises at least one selected from the group consisting of a repeating unit represented by general formula (c1), a repeating unit represented by general formula (c2), a repeating unit represented by general formula (c3), a repeating unit represented by general formula (c4), and a repeating unit represented by general formula (c5):

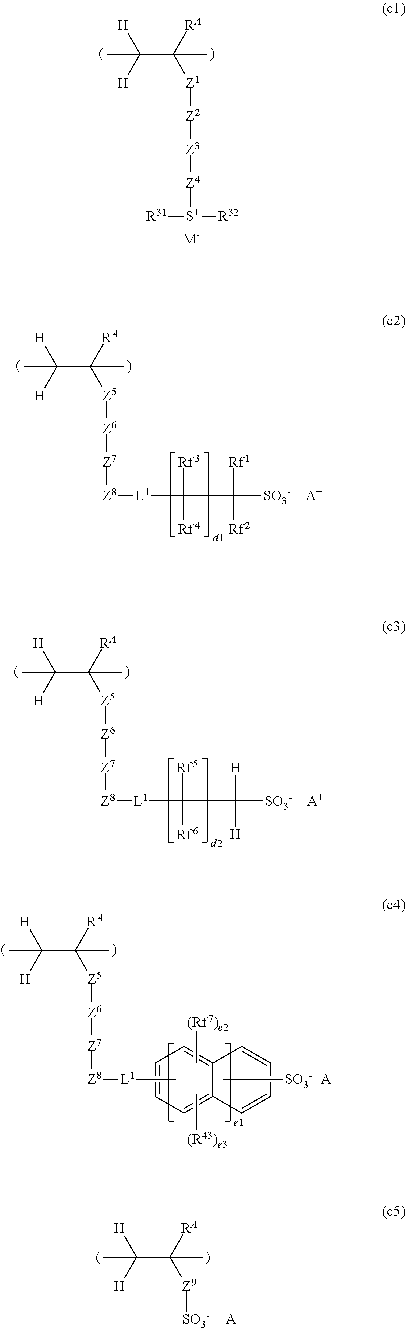

wherein “d1” and “d2” are each independently an integer of 0 to 3;
“e1” is 0 or 1, “e2” is an integer of 0 to 4, and “e3” is an integer of 0 to 4, provided that when “e1” is 0, 0≤e2+e3≤4, and when “e1” is 1, 0≤e2+e3≤6;
R^{A} is each independently a hydrogen atom, a fluorine atom, a methyl group, or a trifluoromethyl group;
Z¹ is a single bond or a phenylene group optionally having a substituent;
Z² is a single bond, **—C(═O)—O—Z²¹—, **—C(═O)—NH—Z²¹—, or **—O—Z²¹—, where Z²¹ is an aliphatic hydrocarbylene group having 1 to 6 carbon atoms, a phenylene group, or a divalent group obtained by combining these groups, and optionally comprises a halogen atom, a carbonyl group, an ester bond, an ether bond, or a hydroxy group;
Z³ is a single bond, an ether bond, an ester bond, a sulfonate ester bond, an amide bond, a sulfonamide bond, a carbonate bond, or a carbamate bond;
Z⁴ is a single bond, an aliphatic hydrocarbylene group having 1 to 6 carbon atoms, a phenylene group, or a divalent group obtained by combining these groups, and optionally comprises a halogen atom, a carbonyl group, an ester bond, an ether bond, or a hydroxy group;
Z⁵ is each independently a single bond, a phenylene group or naphthylene group optionally having a substituent, or *—C(═O)—O—Z⁵¹—, where Z⁵¹ is an aliphatic hydrocarbylene group having 1 to 10 carbon atoms, a phenylene group, or a naphthylene group, wherein the aliphatic hydrocarbylene group optionally comprises a halogen atom, a hydroxy group, an ether bond, an ester bond, or a lactone ring;
Z⁶ is a single bond, an ether bond, an ester bond, a sulfonate ester bond, an amide bond, a sulfonamide bond, a carbonate bond, or a carbamate bond;
each Z⁷ is independently a single bond, ***—Z⁷¹—C(═O)—O—, ***—C(═O)—NH—Z⁷¹—, or ***—O—Z⁷¹—, where Z⁷¹ is a hydrocarbylene group having 1 to 20 carbon atoms and optionally comprising a heteroatom;
each Z⁸ is independently a single bond, ****—Z⁸¹—C(═O)—O—, ****—C(═O)—NH—Z⁸¹—, or ****—O—Z⁸¹—, where Z⁸¹ is a hydrocarbylene group having 1 to 20 carbon atoms and optionally comprising a heteroatom;
Z⁹ is a single bond, a methylene group, an ethylene group, a phenylene group, a fluorinated phenylene group, a phenylene group substituted with a trifluoromethyl group, *—C(═O)—O—Z⁹¹—, *—C(═O)—NH—Z⁹¹—, or *—O—Z⁹¹—, where Z⁹¹ is an aliphatic hydrocarbylene group having 1 to 6 carbon atoms, a phenylene group, a fluorinated phenylene group, or a phenylene group substituted with a trifluoromethyl group, and optionally comprises a carbonyl group, an ester bond, an ether bond, or a hydroxy group;
“*” represents a bond to a carbon atom of a main chain, “**” represents a bond to Z¹, and “***” represents a bond to Z⁶, and “****” represents a bond to Z⁷;
L¹ is a single bond, an ether bond, an ester bond, a carbonyl group, a sulfonate ester bond, a sulfonamide bond, a carbonate bond, or a carbamate bond;
Rf¹ and Rf² are each independently a fluorine atom or a fluorinated saturated hydrocarbyl group having 1 to 6 carbon atoms;
Rf³ and Rf⁴ are each independently a hydrogen atom, a fluorine atom, or a fluorinated saturated hydrocarbyl group having 1 to 6 carbon atoms;
Rf⁵ and Rf⁶ are each independently a hydrogen atom, a fluorine atom, or a fluorinated saturated hydrocarbyl group having 1 to 6 carbon atoms, provided that not all Rf⁵ and Rf⁶ are hydrogen atoms at the same time;
R^{F7} is a fluorine atom, a fluorinated alkyl group having 1 to 6 carbon atoms, a fluorinated alkoxy group having 1 to 6 carbon atoms, or a fluorinated alkylthio group having 1 to 6 carbon atoms;
R⁴¹ and R⁴² are each independently a hydrocarbyl group having 1 to 20 carbon atoms and optionally comprising a heteroatom, or R⁴¹ and R⁴² are optionally bonded to each other to form a ring together with a sulfur atom to which the R⁴¹ and R⁴² are bonded;
R⁴³ is a halogen atom other than a fluorine atom or a hydrocarbyl group having 1 to 20 carbon atoms and optionally comprising a heteroatom, wherein when “e3” is 2, 3, or 4, a plurality of R⁴³ are optionally bonded to each other to form a ring together with carbon atoms to which the R⁴³ are bonded;
M⁻ is a non-nucleophilic counter ion; and
A⁺ is an onium cation.
[11] The chemically amplified resist composition according to any one of [6] to [10], further comprising at least one selected from the group consisting of an organic solvent, a quencher, a photoacid generator other than the photoacid generator, and surfactant.
[12] A patterning process comprising:
forming a resist film on a substrate by using the chemically amplified resist composition according to any one of [6] to [11];
exposing the resist film to high-energy radiation; and
developing the exposed resist film by using a developing solution.
13. The patterning process according to claim 12, wherein the high-energy radiation used is KrF excimer laser, ArF excimer laser, electron beam, or extreme ultraviolet radiation having a wavelength of 3 to 15 nm.

It should be noted that the present invention is not limited to the above-described embodiments. The embodiments are just examples, and any examples that substantially have the same feature and demonstrate the same functions and effects as those in the technical concept disclosed in claims of the present invention are included in the technical scope of the present invention.

1. An onium salt comprising:
an anion represented by general formula (1A); and
a cation represented by general formula (1B): 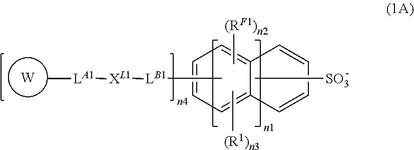
wherein “n1” is 0 or 1, “n2” is an integer of 0 to 4, “n3” is an integer of 0 to 4, provided that when “n1” is 0, 0≤n2+n3≤4, and when “n1” is 1, 0≤n2+n3≤6, and “n4” is 0 or 1;
W is a hydrocarbyl group having 6 to 60 carbon atoms and comprising at least one aromatic ring, wherein the hydrocarbyl group optionally comprises a heteroatom;
R^{F1} is a fluorine atom, a fluorinated saturated hydrocarbyl group having 1 to 6 carbon atoms, a fluorinated saturated hydrocarbyloxy group having 1 to 6 carbon atoms, or a fluorinated saturated hydrocarbylthio group having 1 to 6 carbon atoms;
R¹ is a halogen atom other than fluorine, a nitro group, a hydrocarbyl group having 1 to 20 carbon atoms and optionally comprising a heteroatom, a hydrocarbyloxy group having 1 to 20 carbon atoms and optionally comprising a heteroatom, or a hydrocarbylthio group having 1 to 20 carbon atoms and optionally comprising a heteroatom, wherein when “n3” is 2, 3, or 4, R¹ are the same as or different from each other, and a plurality of R¹ are optionally bonded to each other to form a ring together with carbon atoms to which the R¹ are bonded;
L^{A1} and L^{B1} are each independently a single bond, an ether bond, an ester bond, a sulfonate ester bond, an amide bond, a sulfonamide bond, a carbonate bond, or a carbamate bond;
X^{L1} is a single bond or a hydrocarbylene group having 1 to 40 carbon atoms and optionally comprising a heteroatom, and 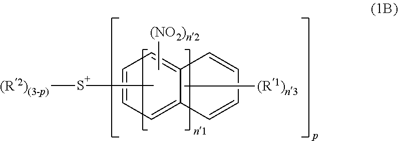
wherein “p” is 1, 2, or 3, “n′1” is 0 or 1, “n′2” is 1 or 2, and “n′3” is an integer of 0 to 6, provided that when “n′1” is 0, 1≤n′2+n′3≤5, and when “n′1” is 1, 1≤n′2+n′3≤7;
R′¹ is a halogen atom, a cyano group, a hydroxy group, a carboxy group, an alkoxycarbonyl group, a hydrocarbyl group having 1 to 20 carbon atoms and optionally comprising a heteroatom, a hydrocarbyloxy group having 1 to 20 carbon atoms and optionally comprising a heteroatom, or a hydrocarbylthio group having 1 to 20 carbon atoms and optionally comprising a heteroatom, wherein when “n′3” is 2 to 6, R′¹ are the same as or different from each other, and two R′¹ are optionally bonded to each other to form a ring together with carbon atoms to which the R′¹ are bonded; and
R′² is a halogen atom or a hydrocarbyl group having 1 to 30 carbon atoms and optionally comprising a heteroatom, wherein when “p” is 1, two R′² are the same as or different from each other, and two of three substituents bonded to S⁺ are optionally bonded to each other to form a ring together with a sulfur atom to which the substituents are bonded.

2. The onium salt according to claim 1, wherein the W is represented by general formula (W-1) or (W-2): 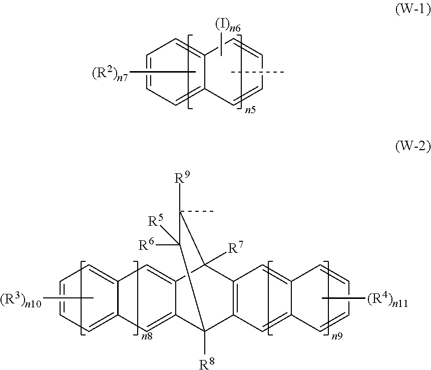
wherein “n5” is 0 or 1, “n6” is an integer of 0 to 4, “n7” is an integer of 1 to 4, provided that when “n5” is 0, 1≤n6+n7≤5, and when “n5” is 1, 1≤n6+n7≤7, “n8” is 0 or 1, “n9” is 0 or 1, “n10” is an integer of 0 to 4, and “n11” is an integer of 0 to 4;
each R² is independently a hydrogen atom, a halogen atom other than an iodine atom, or a hydrocarbyl group having 1 to 20 carbon atoms and optionally comprising a heteroatom;
R³ and R⁴ are each independently a hydrogen atom, a halogen atom, or a hydrocarbyl group having 1 to 20 carbon atoms and optionally comprising a heteroatom;
R⁵ to R⁹ are each independently a hydrogen atom, a halogen atom, or a hydrocarbyl group having 1 to 20 carbon atoms and optionally comprising a heteroatom; and
the dashed line represents a bond to L^{A}i.

3. The onium salt according to claim 1, wherein the anion is represented by general formula (1A-1): 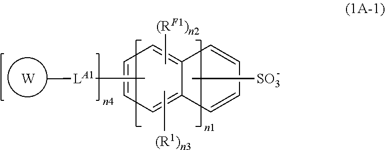
wherein “n1” to “n4”, W, R^{F1}, R¹, and L^{A1} are the same above.

4. The onium salt according to claim 1, wherein the cation is represented by general formula (1B-1): 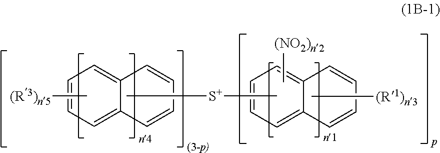
wherein “p”, “n′1” to “n′3”, and R′¹ are the same as above;
“n′4” is 0 or 1, “n′5” is an integer of 0 to 5; and
R′³ is a halogen atom, a cyano group, a hydroxy group, a carboxy group, an alkoxycarbonyl group, a hydrocarbyl group having 1 to 20 carbon atoms and optionally comprising a heteroatom, a hydrocarbyloxy group having 1 to 20 carbon atoms and optionally comprising a heteroatom, or a hydrocarbylthio group having 1 to 20 carbon atoms and optionally comprising a heteroatom, wherein when “n′5” is 2 to 5, R′³ are the same as or different from each other, and two R′³ are optionally bonded to each other to form a ring together with carbon atoms to which the R′³ are bonded.

5. A photoacid generator comprising the onium salt according to claim 1.

6. A photoacid generator comprising the onium salt according to claim 2.

7. A photoacid generator comprising the onium salt according to claim 3.

8. A photoacid generator comprising the onium salt according to claim 4.

9. A chemically amplified resist composition comprising the photoacid generator according to claim 5.

10. The chemically amplified resist composition according to claim 9, further comprising a base polymer comprising a repeating unit represented by either or both of general formulae (a1) and (a2): 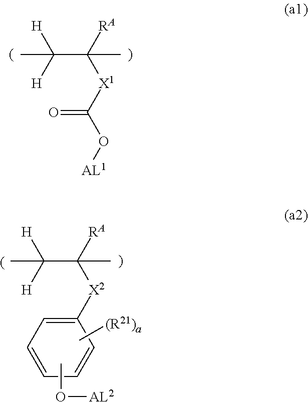
wherein each R^{A} is independently a hydrogen atom, a fluorine atom, a methyl group, or a trifluoromethyl group;
X¹ is a single bond, a phenylene group, a naphthylene group, or *—C(═O)—O—X¹¹—, wherein the phenylene group or naphthylene group is optionally substituted with a hydroxy group, a nitro group, a cyano group, a saturated hydrocarbyl group having 1 to 10 carbon atoms and optionally comprising a fluorine atom, a saturated hydrocarbyloxy group having 1 to 10 carbon atoms and optionally comprising a fluorine atom, or a halogen atom; X¹¹ is a saturated hydrocarbylene group having 1 to 10 carbon atoms, a phenylene group, or a naphthylene group, wherein the saturated hydrocarbylene group optionally comprises a hydroxy group, an ether bond, an ester bond, or a lactone ring;
X² is a single bond or *—C(═O)—O—;
“*” represents a bond to a carbon atom of a main chain;
R²¹ is a halogen atom, a cyano group, a hydroxy group, a nitro group, a hydrocarbyl group having 1 to 20 carbon atoms and optionally comprising a heteroatom, a hydrocarbyloxy group having 1 to 20 carbon atoms and optionally comprising a heteroatom, a hydrocarbylcarbonyl group having 2 to 20 carbon atoms and optionally comprising a heteroatom, a hydrocarbylcarbonyloxy group having 2 to 20 carbon atoms and optionally comprising a heteroatom, or a hydrocarbyloxycarbonyl group having 2 to 20 carbon atoms and optionally comprising a heteroatom;
AL¹ and AL² are each independently an acid labile group; and
“a” is an integer of 0 to 4.

11. The chemically amplified resist composition according to claim 10, wherein the base polymer further comprises a repeating unit represented by general formula (a3): 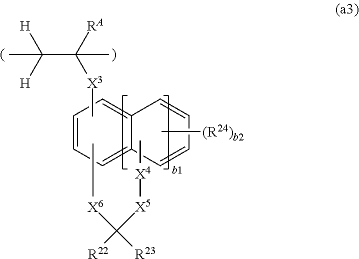
wherein “b1” is 0 or 1, “b2” is an integer of 0 to 3 when the “b1” is 0, and an integer of 0 to 5 when the “b1” is 1;
R^{A} is a hydrogen atom, a fluorine atom, a methyl group, or a trifluoromethyl group;
X³ is a single bond, *—C(═O)—O—, or *—C(═O)—NH—, and “*” represents a bond to a carbon atom of a main chain;
X⁴ is a single bond, an aliphatic hydrocarbylene group having 1 to 4 carbon atoms, a carbonyl group, a sulfonyl group, or a group obtained by combining these groups;
X⁵ and X⁶ are each independently an oxygen atom, or a sulfur atom, provided that X⁴ and X⁶ are bonded to adjacent carbon atoms of an aromatic ring;
R²² and R²³ are each independently a hydrogen atom or a hydrocarbyl group having 1 to 20 carbon atoms and optionally comprising a heteroatom, or R²² and R²³ are optionally bonded to each other to form a ring together with a carbon atom to which the R²² and R²³ are bonded; and
R²⁴ is a halogen atom, a hydroxy group, a cyano group, a nitro group, a hydrocarbyl group having 1 to 20 carbon atoms and optionally comprising a heteroatom, a hydrocarbyloxy group having 1 to 20 carbon atoms and optionally comprising a heteroatom, a hydrocarbyloxycarbonyl group having 2 to 20 carbon atoms and optionally comprising a heteroatom, a hydrocarbylthio group having 1 to 20 carbon atoms and optionally comprising a heteroatom, or —N(R^{24A})(R^{24B}) where R^{24A} and R^{24B} are each independently a hydrogen atom or a hydrocarbyl group having 1 to 6 carbon atoms, and when “b2” is 2 or more, a plurality of R²⁴ are optionally bonded to each other to form a ring together with carbon atoms of an aromatic ring to which the R²⁴ are bonded.

12. The chemically amplified resist composition according to claim 10, wherein the base polymer further comprises a repeating unit represented by either or both of general formulae (b1) and (b2): 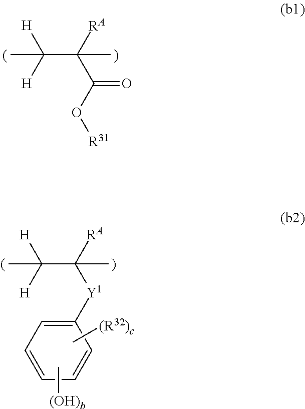
wherein each R^{A} is independently a hydrogen atom, a fluorine atom, a methyl group, or a trifluoromethyl group;
Y¹ is a single bond or *—C(═O)—O—, and “*” represents a bond to a carbon atom of a main chain;
R³¹ is a hydrogen atom or a group having 1 to 20 carbon atoms and comprising at least one structure selected from the group consisting of a hydroxy group other than a phenolic hydroxy group, a cyano group, a carbonyl group, a carboxy group, an ether bond, an ester bond, a sulfonate ester bond, a carbonate bond, a lactone ring, a sultone ring, and a carboxylic anhydride (—C(═O)—O—C(═O)—);
R³² is a halogen atom, a carboxy group, a nitro group, a cyano group, an alkoxycarbonyl group, a hydrocarbyl group having 1 to 20 carbon atoms and optionally comprising a heteroatom, a hydrocarbyloxy group having 1 to 20 carbon atoms and optionally comprising a heteroatom, a hydrocarbylcarbonyl group having 2 to 20 carbon atoms and optionally comprising a heteroatom, a hydrocarbylcarbonyloxy group having 2 to 20 carbon atoms and optionally comprising a heteroatom, or a hydrocarbyloxycarbonyl group having 2 to 20 carbon atoms and optionally comprising a heteroatom, wherein when “c” is 2 or more, R³² are the same as or different from each other; and
“b” is an integer of 1 to 4, and “c” is an integer of 0 to 4, provided that 1≤b+c≤5.

13. The chemically amplified resist composition according to claim 10, wherein the base polymer further comprises at least one selected from the group consisting of a repeating unit represented by general formula (c1), a repeating unit represented by general formula (c2), a repeating unit represented by general formula (c3), a repeating unit represented by general formula (c4), and a repeating unit represented by general formula (c5): 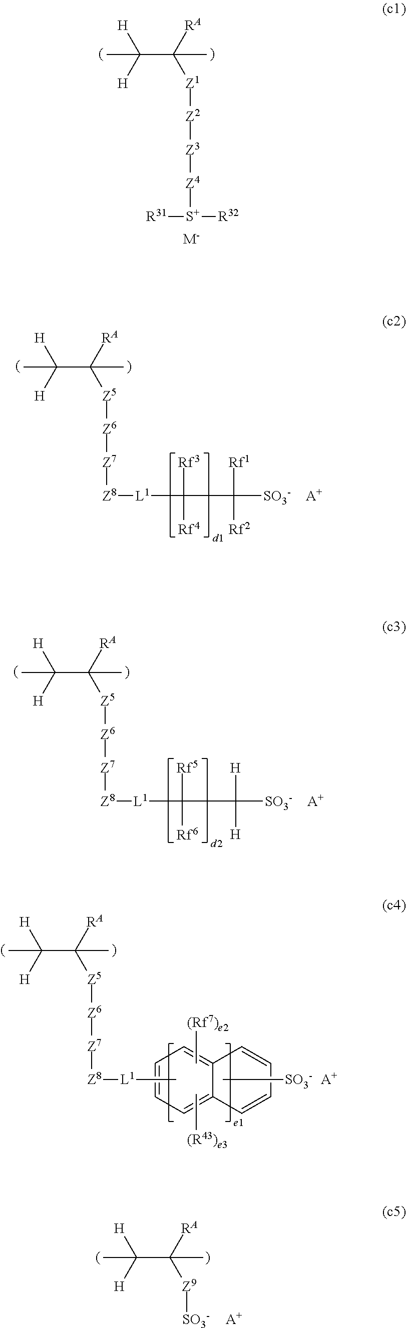
wherein “d1” and “d2” are each independently an integer of 0 to 3;
“e1” is 0 or 1, “e2” is an integer of 0 to 4, and “e3” is an integer of 0 to 4, provided that when “e1” is 0, 0≤e2+e3≤4, and when “e1” is 1, 0≤e2+e3≤6;
R^{A} is each independently a hydrogen atom, a fluorine atom, a methyl group, or a trifluoromethyl group;
Z¹ is a single bond or a phenylene group optionally having a substituent;
Z² is a single bond, **—C(═O)—O—Z²¹—, **—C(═O)—NH—Z²¹—, or **—O—Z²¹—, where Z²¹ is an aliphatic hydrocarbylene group having 1 to 6 carbon atoms, a phenylene group, or a divalent group obtained by combining these groups, and optionally comprises a halogen atom, a carbonyl group, an ester bond, an ether bond, or a hydroxy group;
Z³ is a single bond, an ether bond, an ester bond, a sulfonate ester bond, an amide bond, a sulfonamide bond, a carbonate bond, or a carbamate bond;
Z⁴ is a single bond, an aliphatic hydrocarbylene group having 1 to 6 carbon atoms, a phenylene group, or a divalent group obtained by combining these groups, and optionally comprises a halogen atom, a carbonyl group, an ester bond, an ether bond, or a hydroxy group;
Z⁵ is each independently a single bond, a phenylene group or naphthylene group optionally having a substituent, or *—C(═O)—O—Z⁵¹—, where Z⁵¹ is an aliphatic hydrocarbylene group having 1 to 10 carbon atoms, a phenylene group, or a naphthylene group, wherein the aliphatic hydrocarbylene group optionally comprises a halogen atom, a hydroxy group, an ether bond, an ester bond, or a lactone ring;
Z⁶ is a single bond, an ether bond, an ester bond, a sulfonate ester bond, an amide bond, a sulfonamide bond, a carbonate bond, or a carbamate bond;
each Z⁷ is independently a single bond, ***—Z⁷¹—C(═O)—O—, ***—C(═O)—NH—Z⁷¹—, or ***—O—Z⁷¹—, where Z⁷¹ is a hydrocarbylene group having 1 to 20 carbon atoms and optionally comprising a heteroatom;
each Z⁸ is independently a single bond, ****—Z⁸¹—C(═O)—O—, ****—C(═O)—NH—Z⁸¹—, or ****—O—Z⁸¹—, where Z⁸¹ is a hydrocarbylene group having 1 to 20 carbon atoms and optionally comprising a heteroatom;
Z⁹ is a single bond, a methylene group, an ethylene group, a phenylene group, a fluorinated phenylene group, a phenylene group substituted with a trifluoromethyl group, *—C(═O)—O—Z⁹¹—, *—C(═O)—NH—Z⁹¹—, or *—O—Z⁹¹—, where Z⁹¹ is an aliphatic hydrocarbylene group having 1 to 6 carbon atoms, a phenylene group, a fluorinated phenylene group, or a phenylene group substituted with a trifluoromethyl group, and optionally comprises a carbonyl group, an ester bond, an ether bond, or a hydroxy group;
“*” represents a bond to a carbon atom of a main chain, “**” represents a bond to Z¹, and “***” represents a bond to Z⁶, and “****” represents a bond to Z⁷;
L¹ is a single bond, an ether bond, an ester bond, a carbonyl group, a sulfonate ester bond, a sulfonamide bond, a carbonate bond, or a carbamate bond;
Rf¹ and Rf² are each independently a fluorine atom or a fluorinated saturated hydrocarbyl group having 1 to 6 carbon atoms;
Rf³ and Rf⁴ are each independently a hydrogen atom, a fluorine atom, or a fluorinated saturated hydrocarbyl group having 1 to 6 carbon atoms;
Rf⁵ and Rf⁶ are each independently a hydrogen atom, a fluorine atom, or a fluorinated saturated hydrocarbyl group having 1 to 6 carbon atoms, provided that not all Rf⁵ and Rf⁶ are hydrogen atoms at the same time;
R^{F7} is a fluorine atom, a fluorinated alkyl group having 1 to 6 carbon atoms, a fluorinated alkoxy group having 1 to 6 carbon atoms, or a fluorinated alkylthio group having 1 to 6 carbon atoms;
R⁴¹ and R⁴² are each independently a hydrocarbyl group having 1 to 20 carbon atoms and optionally comprising a heteroatom, or R⁴¹ and R⁴² are optionally bonded to each other to form a ring together with a sulfur atom to which the R⁴¹ and R⁴² are bonded;
R⁴³ is a halogen atom other than a fluorine atom or a hydrocarbyl group having 1 to 20 carbon atoms and optionally comprising a heteroatom, wherein when “e3” is 2, 3, or 4, a plurality of R⁴³ are optionally bonded to each other to form a ring together with carbon atoms to which the R⁴³ are bonded;
M⁻ is a non-nucleophilic counter ion; and
A⁺ is an onium cation.

14. The chemically amplified resist composition according to claim 9, further comprising at least one selected from the group consisting of an organic solvent, a quencher, a photoacid generator other than the photoacid generator, and surfactant.

15. A patterning process comprising:
forming a resist film on a substrate by using the chemically amplified resist composition according to claim 9;
exposing the resist film to high-energy radiation; and
developing the exposed resist film by using a developing solution.

16. The patterning process according to claim 15, wherein the high-energy radiation used is KrF excimer laser, ArF excimer laser, electron beam, or extreme ultraviolet radiation having a wavelength of 3 to 15 nm.
